SEQUENCE LISTING (73 sequences)

SEQ ID NO: 1 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 2 - protein, 330 aa, Homo sapiens [sequence omitted]

SEQ ID NO: 3 - protein, 107 aa, Homo sapiens [sequence omitted]

SEQ ID NO: 4 - protein, 6 aa, synthetic construct
HHHHHH

SEQ ID NO: 5 - protein, 5 aa, synthetic construct
  VARIANT 3: note = Any amino acid
LPXTG

SEQ ID NO: 6 - protein, 123 aa, synthetic construct
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 7 - protein, 107 aa, synthetic construct
EIVMTQSPSSVSASVGDRVAITCRASQGISSWLAWYQQKPGKAPKLLIYAASSLQSGVPSRFSGSGSGTDFTLTISSLQPEDFATYYCQQSYSTPLTFGGGTKVDIK

SEQ ID NO: 8 - protein, 123 aa, synthetic construct
EVQLLESGGGVVQHGRSLRLSCAASGFTFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 9 - protein, 107 aa, synthetic construct; identical to SEQ ID NO: 7

SEQ ID NO: 10 - protein, 122 aa, synthetic construct
EVQLLESGGGVVQLGGPDSPVQPLDSPFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 11 - protein, 107 aa, synthetic construct; identical to SEQ ID NO: 7

SEQ ID NO: 12 - protein, 123 aa, synthetic construct
EVQLLESGGGVVQRGRSLRLSCAASGFTFSSYGMHWVRQAPGKGLEWVAVISYDGSNKYYADSVKGRFTISRDNSKNTLYLPMNSLRAEDTAVYYCARPRAYYGAYGSSFDYWGQGTQVTVSS

SEQ ID NO: 13 - protein, 107 aa, synthetic construct; identical to SEQ ID NO: 7

SEQ ID NO: 14 - protein, 123 aa, synthetic construct
EVQLLESGGGVVQPGRSLRLSCAASGFTFSSYAMHWVRQAPGKGLEWVAVISYDGSNKYYADSVKGRFIISRDNSKNTVYLQMNSLRAEDTAVYYCARPTYVFTYTGSSFDYWGQGTQVTVSS

SEQ ID NO: 15 - protein, 107 aa, synthetic construct
DIQVTQSPSSLSASLGDTVSITCRASRGLTDSVAWYQQKPGQAPKLLIYAASTLQSGVPSRFGGSGSGSYFTLTITSLQPEDVATYYCQNYKSAPWTFGQGTKVEIK

SEQ ID NO: 16 - protein, 123 aa, synthetic construct
EVQLLESGGGVVQPGRSLRLSCAASGFTFSSYGMHWVRQAPGKGLEWVAVISYDGSNKYYADSVKGRFTISRDNSKNTLYLHMNSLRAEDTAVYYCARPRAYYGAYGSSFDYWGQGTQVTVSS

SEQ ID NO: 17 - protein, 107 aa, synthetic construct; identical to SEQ ID NO: 7

SEQ ID NO: 18 - protein, 123 aa, synthetic construct
EVQLLESGGGVVQRGRSLRLSCAASGFTFSSYGMHWVRQAPGKGLEWVAVISYDGSNKYYADSVKGRFTISRDNSKNTLYLQMNSLRAEDTAVYYCARPRAYYGAYGSSFDYWGQGTQVTVSS

SEQ ID NO: 19 - protein, 107 aa, synthetic construct; identical to SEQ ID NO: 7

SEQ ID NO: 20 - protein, 123 aa, synthetic construct
EVQLLESGGGVVQPGRSLRLSCAASGFTFSSYGMHWVRQAPGKGLEWVAVISYDGSNKYYADSVKGRFTISRPNSKNTLYLQMNSLRAEDTAVYYCARPRAYYGAYGSSFDYWGQGTQVTVSS

SEQ ID NO: 21 - protein, 107 aa, synthetic construct; identical to SEQ ID NO: 7

SEQ ID NO: 22 - protein, 123 aa, synthetic construct
EVQLLESGGGVVQPGRSLRLSCAASGFTFSSYGMHWVRQAPGKGLEWVAVISYDGSNKYYADSVKGRFTISRHNSKNTLYLQMNSLRAEDTAVYYCARPRAYYGAYGSSFDYWGQGTQVTVSS

SEQ ID NO: 23 - protein, 107 aa, synthetic construct; identical to SEQ ID NO: 7

SEQ ID NO: 24 - protein, 123 aa, synthetic construct
EVQLLESGGGVVQPERSLRLSCAASGFTFSSYGMHWVRQAPGKGLEWVAVISYDGSNKYYADSVKGRFTISRANSKNTLYLQMNSLRAEDTAVYYCARPRAYYGAYGSSFDYWGQGTQVTVSS

SEQ ID NO: 25 - protein, 107 aa, synthetic construct; identical to SEQ ID NO: 7

SEQ ID NO: 26 - protein, 123 aa, synthetic construct
EVQLLESGGGVVQPGRSLRLSCAASGFTFSSYGMHWVRQAPGKGLEWVAVISYDGSNKYYADSVKGRFTISRANSKNTLYLQMNSLRAEDTAVYYCARPRAYYGAYGSSFDYWGQGTQVTVSS

SEQ ID NO: 27 - protein, 107 aa, synthetic construct; identical to SEQ ID NO: 7

SEQ ID NO: 28 - protein, 123 aa, synthetic construct
EVQLLESGGGVVQPGRSLRLSCAASGFTFSSYGMHWVRQAPGKGLEWVAVISYDGSNKYYADSVKGRFTISTHNSKNTLYLQMNSLRAEDTAVYYCARPRAYYGAYGSSFDYWGQGTQVTVSS

SEQ ID NO: 29 - protein, 107 aa, synthetic construct; identical to SEQ ID NO: 7

SEQ ID NO: 30 - protein, 5 aa, synthetic construct
SYGMH

SEQ ID NO: 31 - protein, 17 aa, synthetic construct
VISYDGSNKYYADSVKG

SEQ ID NO: 32 - protein, 14 aa, synthetic construct
PRAYYGAYGSSFDY

SEQ ID NO: 33 - protein, 11 aa, synthetic construct
RASQGISSWLA

SEQ ID NO: 34 - protein, 7 aa, synthetic construct
AASSLQS

SEQ ID NO: 35 - protein, 9 aa, synthetic construct
QQSYSTPLT

SEQ ID NO: 36 - protein, 5 aa, synthetic construct
SYAMH

SEQ ID NO: 37 - protein, 14 aa, synthetic construct
PTYVFTYTGSSFDY

SEQ ID NO: 38 - protein, 11 aa, synthetic construct
RASRGLTDSVA

SEQ ID NO: 39 - protein, 7 aa, synthetic construct
AASTLQS

SEQ ID NO: 40 - protein, 8 aa, synthetic construct
QNYKSAPW

SEQ ID NO: 41 - protein, 330 aa, Homo sapiens [sequence omitted]

SEQ ID NO: 42 - protein, 107 aa, Homo sapiens [sequence omitted]

SEQ ID NO: 43 - protein, 4 aa, synthetic construct
GGFG

SEQ ID NO: 44 - protein, 4 aa, synthetic construct
  VARIANT 1: note = Residue attached to a sugar unit
GGFG

SEQ ID NO: 45 - protein, 4 aa, synthetic construct
  VARIANT 1: note = Residue attached to a carboxylated unit
GGFG

SEQ ID NO: 46 - protein, 5 aa, synthetic construct
  SITE 1: note = Residue attached to Polyethylene glycol
KGGFG

SEQ ID NO: 47 - protein, 4 aa, synthetic construct
GFLG

SEQ ID NO: 48 - protein, 4 aa, synthetic construct
GGFK

SEQ ID NO: 49 - protein, 4 aa, synthetic construct
  SITE 4: note = (4-aminophenyl)methanol modified residue
GGVK

SEQ ID NO: 50 - protein, 4 aa, synthetic construct
  SITE 4: note = (4-aminophenyl)methanol 2,2',2'',2'''-(ethane-1,2-diylbis(azanetriyl))tetraacetic acid modified residue
GGVK

SEQ ID NO: 51 - protein, 4 aa, synthetic construct
  SITE 1: note = N2-(N2-(N6,N6-bis((2S,3R,4R,5R)-2,3,4,5,6-pentahydr oxyhexyl)-L-lysyl)-N6-(3-carboxypropanoyl)-L-lysyl)-N6,N6- bis((2S,3R,4R,5R)-2,3,4,5,6-pentahydroxyhexyl)-L-lysine modified residue
GGFG

SEQ ID NO: 52 - protein, 5 aa, synthetic construct
  SITE 5: note = 1-amino-3,6,9,12-tetraoxapentadecan-15-oic acid modified residue
GGGGG

SEQ ID NO: 53 - protein, 5 aa, synthetic construct
  SITE 1: note = 3-(2-(2-(2-(2,5-dioxo-2,5-dihydro-1H-pyrrol-1-yl)et hoxy)ethoxy)ethoxy)propanoic acid (2R,3R,4R,5S)-hexane-1,2 ,3,4,5-pentanol-(2R,3R,4R,5S)-hexane-1,2,3,4,5-pentanol modified residue
  SITE 3: note = (2R,3R,4R,5S)-hexane-1,2,3,4,5-pentanol-(2R,3R,4R,5 S)-hexane-1,2,3,4,5-pentanol modified residue
  SITE 5: note = (4-aminophenyl)methanol modified L-citrulline
KKKVX

SEQ ID NO: 54 - protein, 4 aa, synthetic construct
  SITE 1: note = N2-(N2-(N2-(2-bromoacetyl)-N6,N6-bis((2S,3R,4R,5R)- 2,3,4,5,6-pentahydroxyhexyl)-L-lysyl)-N6-(3-carboxypropano yl)-L-lysyl)-N6,N6-bis((2S,3R,4R,5R)-2,3,4,5,6-pentahydrox yhexyl)-L-lysine modified residue
GGFG

SEQ ID NO: 55 - protein, 5 aa, synthetic construct
  SITE 5: note = (42S,43R,44R,45R)-N-((S)-1-amino-17-(((S)-1-(((S)-1 -((4-(hydroxymethyl)phenyl)amino)-1-oxo-5-ureidopentan-2-y l)amino)-3-methyl-1-oxobutan-2-yl)carbamoyl)-15-oxo-3,6,9, 12-tetraoxa-16-azahenicosan-21-yl)-42,43,44,45,46-pentahyd roxy-40-((2S,3R,4R,5R)-2,3,4,5,6-pentahydroxyhexyl)-4,7,10 ,13,16,19,22,25,28,31,34,37-dodecaoxa-40-azahexatetraconta namide modified residue
GGGGG

SEQ ID NO: 56 - protein, 4 aa, synthetic construct
  SITE 1: note = 6-(2,5-dioxo-2,5-dihydro-1H-pyrrol-1-yl)hexanoic acid modified residue
  SITE 4: note = 2,2',2'',2'''-(ethane-1,2-diylbis(azanetriyl))tetra acetic acid (4-aminophenyl)methanol modified residue
GGVK

SEQ ID NO: 57 - protein, 5 aa, synthetic construct
  SITE 5: note = 4-((2S,5S,8S,55S,56R,57R,58R)-8-(1-amino-3,6,9,12-t etraoxapentadecan-15-amido)-55,56,57,58,59-pentahydroxy-5- isopropyl-4,7,14-trioxo-53-((2S,3R,4R,5R)-2,3,4,5,6-pentah ydroxyhexyl)-2-(3-ureidopropyl)-17,20,23,26,29,32,35,38,41 ,44,47,50-dodecaoxa-3,6,13,53-tetraazanonapentacontanamido )benzyl ((S)-1-(((S)-1-(((3S,4S,5S)-1-((S)-2-((1R,2R)-3-(( (1R,2S)-1-hydroxy-1-phenylpropan-2-yl)amino)-1-methoxy-2-m ethyl-3-oxopropyl)pyrrolidin-1-yl)-3-methoxy-5-methyl-1-ox oheptan-4-yl)(methyl)amino)-3-methyl-1-oxobutan-2-yl)amino )-3-methyl-1-oxobutan-2-yl)(methyl)carbamate modified residue
GGGGG

SEQ ID NO: 58 - protein, 5 aa, synthetic construct
  SITE 5: note = 4-((2S,5S,8S,55S,56R,57R,58R)-8-(1-amino-3,6,9,12-t etraoxapentadecan-15-amido)-55,56,57,58,59-pentahydroxy-5- isopropyl-4,7,14-trioxo-53-((2S,3R,4R,5R)-2,3,4,5,6-pentah ydroxyhexyl)-2-(3-ureidopropyl)-17,20,23,26,29,32,35,38,41 ,44,47,50-dodecaoxa-3,6,13,53-tetraazanonapentacontanamido )benzyl ((1S,9S)-9-ethyl-5-fluoro-9-hydroxy-4-methyl-10,13 -dioxo-2,3,9,10,13,15-hexahydro-1H,12H-benzo[de]pyrano[3', 4':6,7]indolizino[1,2-b]quinolin-1-yl)carbamate modified residue
GGGGG

SEQ ID NO: 59 - protein, 4 aa, synthetic construct
  SITE 1: note = 6-(2,5-dioxo-2,5-dihydro-1H-pyrrol-1-yl)hexanoic acid modified residue
  SITE 4: note = 4-aminobenzyl ((1S,9S)-9-ethyl-5-fluoro-9-hydroxy-4 -methyl-10,13-dioxo-2,3,9,10,13,15-hexahydro-1H,12H-benzo[ de]pyrano[3',4':6,7]indolizino[1,2-b]quinolin-1-yl)carbama te 2,2',2'',2'''-(ethane-1,2-diylbis(azanetriyl))tetraacetic acid modified residue
GGVK

SEQ ID NO: 60 - protein, 5 aa, synthetic construct
  SITE 5: note = 4-((2S,5S,8S,55S,56R,57R,58R)-8-(1-amino-3,6,9,12-t etraoxapentadecan-15-amido)-55,56,57,58,59-pentahydroxy-5- isopropyl-4,7,14-trioxo-53-((2S,3R,4R,5R)-2,3,4,5,6-pentah ydroxyhexyl)-2-(3-ureidopropyl)-17,20,23,26,29,32,35,38,41 ,44,47,50-dodecaoxa-3,6,13,53-tetraazanonapentacontanamido )benzyl ((S)-1-(((S)-1-(((3S,4S,5S)-1-((S)-2-((1R,2R)-3-(( (1R,2S)-1-hydroxy-1-phenylpropan-2-yl)amino)-1-methoxy-2-m ethyl-3-oxopropyl)pyrrolidin-1-yl)-3-methoxy-5-methyl-1-ox oheptan-4-yl)(methyl)amino)-3-methyl-1-oxobutan-2-yl)amino )-3-methyl-1-oxobutan-2-yl)(methyl)carbamate modified residue
GGGGG

SEQ ID NO: 61 - protein, 5 aa, synthetic construct
  SITE 5: note = 4-((2S,5S,8S,55S,56R,57R,58R)-8-(1-amino-3,6,9,12-t etraoxapentadecan-15-amido)-55,56,57,58,59-pentahydroxy-5- isopropyl-4,7,14-trioxo-53-((2S,3R,4R,5R)-2,3,4,5,6-pentah ydroxyhexyl)-2-(3-ureidopropyl)-17,20,23,26,29,32,35,38,41 ,44,47,50-dodecaoxa-3,6,13,53-tetraazanonapentacontanamido )benzyl ((1S,9S)-9-ethyl-5-fluoro-9-hydroxy-4-methyl-10,13 -dioxo-2,3,9,10,13,15-hexahydro-1H,12H-benzo[de]pyrano[3', 4':6,7]indolizino[1,2-b]quinolin-1-yl)carbamate modified residue
GGGGG

SEQ ID NO: 62 - protein, 4 aa, synthetic construct
  SITE 1: note = 6-(3-Ab-2,5-dioxopyrrolidin-1-yl)hexanoic acid modified residue
  SITE 4: note = 4-aminobenzyl ((1S,9S)-9-ethyl-5-fluoro-9-hydroxy-4 -methyl-10,13-dioxo-2,3,9,10,13,15-hexahydro-1H,12H-benzo[ de]pyrano[3',4':6,7]indolizino[1,2-b]quinolin-1-yl)carbama te 2,2',2'',2'''-(ethane-1,2-diylbis(azanetriyl))tetraacetic acid modified residue
GGVK

SEQ ID NO: 63 - protein, 4 aa, synthetic construct
  SITE 1: note = 6-(2,5-dioxo-2,5-dihydro-1H-pyrrol-1-yl)hexanoic acid modified residue
  SITE 4: note = 4-aminobenzyl ((1S,9S)-9-ethyl-5-fluoro-9-hydroxy-4 -methyl-10,13-dioxo-2,3,9,10,13,15-hexahydro-1H,12H-benzo[ de]pyrano[3',4':6,7]indolizino[1,2-b]quinolin-1-yl)carbama te 2,2',2'',2'''-(ethane-1,2-diylbis(azanetriyl))tetraacetic acid modified residue
GGVK

SEQ ID NO: 64 - protein, 4 aa, synthetic construct
  SITE 1: note = 6-(2,5-dioxo-2,5-dihydro-1H-pyrrol-1-yl)hexanoic acid modified residue
  SITE 4: note = 4-aminobenzyl ((1S,9S)-9-ethyl-5-fluoro-9-hydroxy-4 -methyl-10,13-dioxo-2,3,9,10,13,15-hexahydro-1H,12H-benzo[ de]pyrano[3',4':6,7]indolizino[1,2-b]quinolin-1-yl)carbama te diphenyl(p-tolyl)methyl modified residue
GGVK

SEQ ID NO: 65 - protein, 4 aa, synthetic construct
  SITE 1: note = 6-(2,5-dioxo-2,5-dihydro-1H-pyrrol-1-yl)hexanoic acid modified residue
  SITE 4: note = 4-aminobenzyl ((1S,9S)-9-ethyl-5-fluoro-9-hydroxy-4 -methyl-10,13-dioxo-2,3,9,10,13,15-hexahydro-1H,12H-benzo[ de]pyrano[3',4':6,7]indolizino[1,2-b]quinolin-1-yl)carbama te modified residue
GGVK

SEQ ID NO: 66 - protein, 4 aa, synthetic construct
  SITE 4: note = 4-aminobenzyl ((1S,9S)-9-ethyl-5-fluoro-9-hydroxy-4 -methyl-10,13-dioxo-2,3,9,10,13,15-hexahydro-1H,12H-benzo[ de]pyrano[3',4':6,7]indolizino[1,2-b]quinolin-1-yl)carbama te diphenyl(p-tolyl)methyl modified residue
GGVK

SEQ ID NO: 67 - protein, 4 aa, synthetic construct
  SITE 1: note = (9H-fluoren-9-yl)methyl oxyxcarbonyl modified residue
  SITE 4: note = 4-aminobenzyl ((1S,9S)-9-ethyl-5-fluoro-9-hydroxy-4 -methyl-10,13-dioxo-2,3,9,10,13,15-hexahydro-1H,12H-benzo[ de]pyrano[3',4':6,7]indolizino[1,2-b]quinolin-1-yl)carbama te diphenyl(p-tolyl)methyl modified residue
GGVK

SEQ ID NO: 68 - protein, 5 aa, synthetic construct
  SITE 1: note = (9H-fluoren-9-yl)methyl oxyxcarbonyl modified residue
  SITE 5: note = 4-((2S,5S,8S,55S,56R,57R,58R)-8-(1-amino-3,6,9,12-t etraoxapentadecan-15-amido)-55,56,57,58,59-pentahydroxy-5- isopropyl-4,7,14-trioxo-53-((2S,3R,4R,5R)-2,3,4,5,6-pentah ydroxyhexyl)-2-(3-ureidopropyl)-17,20,23,26,29,32,35,38,41 ,44,47,50-dodecaoxa-3,6,13,53-tetraazanonapentacontanamido )benzyl ((1S,9S)-9-ethyl-5-fluoro-9-hydroxy-4-methyl-10,13 -dioxo-2,3,9,10,13,15-hexahydro-1H,12H-benzo[de]pyrano[3', 4':6,7]indolizino[1,2-b]quinolin-1-yl)carbamate modified residue
GGGGG

SEQ ID NO: 69 - protein, 5 aa, synthetic construct
  SITE 1: note = (9H-fluoren-9-yl)methyl oxyxcarbonyl modified residue
  SITE 5: note = 4-((2S,5S,8S,55S,56R,57R,58R)-8-(1-amino-3,6,9,12-t etraoxapentadecan-15-amido)-55,56,57,58,59-pentahydroxy-5- isopropyl-4,7,14-trioxo-53-((2S,3R,4R,5R)-2,3,4,5,6-pentah ydroxyhexyl)-2-(3-ureidopropyl)-17,20,23,26,29,32,35,38,41 ,44,47,50-dodecaoxa-3,6,13,53-tetraazanonapentacontanamido )benzyl ((S)-1-(((S)-1-(((3S,4S,5S)-1-((S)-2-((1R,2R)-3-(( (1R,2S)-1-hydroxy-1-phenylpropan-2-yl)amino)-1-methoxy-2-m ethyl-3-oxopropyl)pyrrolidin-1-yl)-3-methoxy-5-methyl-1-ox oheptan-4-yl)(methyl)amino)-3-methyl-1-oxobutan-2-yl)amino )-3-methyl-1-oxobutan-2-yl)(methyl)carbamate modified residue
GGGGG

SEQ ID NO: 70 - protein, 4 aa, synthetic construct
  SITE 1: note = N2-(N2-(N6,N6-bis((2S,3R,4R,5R)-2,3,4,5,6-pentahydr oxyhexyl)-L-lysyl)-N6-(3-carboxypropanoyl)-L-lysyl)-N6,N6- bis((2S,3R,4R,5R)-2,3,4,5,6-pentahydroxyhexyl)-L-lysine modified residue
GGFG

SEQ ID NO: 71 - protein, 5 aa, synthetic construct
  SITE 1: note = (2R,3R,4R,5S)-hexane-1,2,3,4,5-pentanol-(2R,3R,4R,5 S)-hexane-1,2,3,4,5-pentanol modified residue
  SITE 2: note = (15-amino-15-oxo-3,6,9,12-tetraoxapentadecyl)phosph onic acid modified residue
  SITE 3: note = (2R,3R,4R,5S)-hexane-1,2,3,4,5-pentanol-(2R,3R,4R,5 S)-hexane-1,2,3,4,5-pentanol modified residue
  SITE 5: note = (4-aminophenyl)methanol modified L-citrulline
KKKVX

SEQ ID NO: 72 - protein, 5 aa, synthetic construct
  SITE 1: note = (2R,3R,4R,5S)-hexane-1,2,3,4,5-pentanol-(2R,3R,4R,5 S)-hexane-1,2,3,4,5-pentanol modified residue
  SITE 2: note = (15-amino-15-oxo-3,6,9,12-tetraoxapentadecyl)phosph onic acid modified residue
  SITE 3: note = (2R,3R,4R,5S)-hexane-1,2,3,4,5-pentanol-(2R,3R,4R,5 S)-hexane-1,2,3,4,5-pentanol modified residue
  SITE 5: note = 4-aminobenzyloxycarbonyl modified L-citrulline
KKKVX

SEQ ID NO: 73 - protein, 5 aa, synthetic construct
  SITE 1: note = (9H-fluoren-9-yl)methyl oxyxcarbonyl modified residue
  SITE 5: note = (2R,3R,4R,5S,52S)-52-(1-amino-3,6,9,12-tetraoxapent adecan-15-amido)-1,2,3,4,5-pentahydroxy-46-oxo-7-((2S,3R,4 R,5R)-2,3,4,5,6-pentahydroxyhexyl)-10,13,16,19,22,25,28,31 ,34,37,40,43-dodecaoxa-7,47-diazatripentacontan-53-oic acid modified residue
GGGGG

(19) United States
(12) Patent Application Publication
(10) Pub. No.: US 2024/0245796 A1
(43) Pub. Date: Jul. 25, 2024

(54) LINKERS, DRUG LINKERS AND CONJUGATES THEREOF AND METHODS OF USING THE SAME

(71) Applicant: ProfoundBio US Co., Woodinville, WA (US)

(72) Inventors: Xiao SHANG, Las Vegas, NV (US); Haidong LIU, Suzhou (CN); Julia GAVRILYUK, San Diego, CA (US); Baiteng ZHAO, Bellevue, WA (US); Tae HAN, Woodinville, WA (US)

(21) Appl. No.: 18/595,275

(22) Filed: Mar. 4, 2024

Related U.S. Application Data
(63) Continuation of application No. PCT/CN2022/104174, filed on Jul. 6, 2022.

(30) Foreign Application Priority Data
Jul. 6, 2021 (WO) PCT/CN2021/104618
Jul. 4, 2022 (CN) 202210777240.7

(51) Int. Cl. A61K 47/68

(52) U.S. Cl. CPC A61K 47/68037; A61K 47/68031; A61K 47/6889

(57) ABSTRACT

The present invention provides Polar units, Linker intermediates, Linkers, Drug-Linkers and Conjugates thereof.

### CROSS-REFERENCE

This patent application is a continuation application of International Application No. PCT/CN2022/104174, filed on Jul. 6, 2022, which claims the benefit of International Application No. PCT/CN2021/104618, filed on Jul. 6, 2021 and Chinese patent application 20210777240.7, filed on Jul. 4, 2022; each of which is herein incorporated by reference in its entirety.

### SEQUENCE LISTING

The instant application contains a Sequence Listing which has been submitted electronically. The contents of the electronic Sequence Listing (65895⁻⁷⁰²_301_SL.xml; Size: 92,358 bytes; and Date of Creation: Oct. 31, 2023) is herein incorporated by reference in its entirety.

### BACKGROUND

A great deal of interest has surrounded the use of monoclonal antibodies (mAbs) for the targeted delivery of cytotoxic agents to cells associated with disease, such as cancer cells and other cells, in the form of antibody drug conjugates (or ADCs). The design of antibody drug conjugates, by attaching a cytotoxic agent, immune modulatory agent or other agent (collectively a “drug”) to an antibody, typically via a linker, involves consideration of a variety of factors. These factors include the identity and location of the chemical group for attachment of the drug, the mechanism of drug release, the structural element(s) (if any) providing release of the drug, and structural modification of the released free drug, if any. If the drug is released in the extracellular environment, the released form of the drug must able to reach its target. If the drug is to be released after antibody internalization, the structural elements and mechanism of drug release must be consonant with the intracellular trafficking of the conjugate.

Another important factor in the design of antibody drug conjugates is the amount of drug that can be delivered per targeting agent (i.e., the number of drugs attached to each targeting agent (e.g., an antibody), referred to as the drug load or drug loading). Historically, assumptions were that higher drugs loads were superior to lower drug loads (e.g., 8-loads vs 4-loads). The rationale was that higher loaded conjugates would deliver more drug (e.g., cytotoxic agent) to the target cells. This rationale was supported by the observations that conjugates with higher drug loadings were more active against cell lines in vitro. Certain later studies revealed, however, that this assumption was not confirmed in animal models. Conjugates having drug loads of 4 or 8 of certain auristatins were observed to have similar activities in mouse models. See, e.g., Hamblett et al., Clinical Cancer Res. 10:7063⁻⁷⁰ (2004). Hamblett et al. further reported that the higher loaded ADCs were cleared more quickly from circulation in animal models. This faster clearance suggested a PK liability for higher loaded species as compared to lower loaded species. See Hamblett et al. In addition, higher loaded conjugates had lower maximum tolerated doses (MTDs) in mice, and as a result had narrower reported therapeutic indices. Id. In contrast, ADCs with a drug loading of 2 at engineered sites in a monoclonal antibody were reported to have the same or better PK and therapeutic indices as compared to certain 4-loaded ADCs. For example, see Junutula et al., Clinical Cancer Res. 16:4769 (2010). Thus, recent trends are to develop ADCs with low drug loadings.

There is a need, therefore, for antibody drug conjugate formats (and more generally for formats for other conjugates), that allow for higher drug loading, but that maintain other characteristics of lower loaded conjugates, such as favorable PK properties. Surprisingly, the present invention addresses those needs.

### SUMMARY OF THE INVENTION

Provided herein are Linkers having hydrophilic characteristics that maintain the intrinsic properties of antibodies conjugated with the Linkers and drugs. In particular, the Linkers aid in maintaining the hydrophilic properties of the antibodies when conjugated at higher drug loading and/or to hydrophobic drugs and other agents. Also provided are Drug-Linkers and conjugates comprising the Linkers, as well as methods of using such conjugates for the treatment of cancer and other diseases.

In some embodiments, provided is a Linker intermediate having the following formula (V):

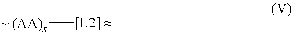

or a salt thereof, wherein:
   AA is an Amino Acid unit having from 1 to 12 amino acid subunits;
   s is 0 or 1;
   L2 is a Linker Subunit having from 1 to 4 attachment sites for a Drug unit; and
   each wavy (˜) line indicates an attachment site for a Stretcher Unit and the double wavy (≈) line indicates an attachment site for a Drug Unit,
wherein at least one Polar unit is present within the Amino Acid unit, the Linker Subunit, or both, and wherein the Polar unit(s) is selected from Sugar units, PEG units, Carboxyl units, and combinations thereof.

In some embodiments, provided is a Linker having the following formula (I):

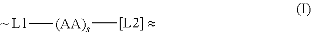

or a salt thereof, wherein:
L1 is a Stretcher unit having an attachment site for a Targeting unit;
AA is an Amino Acid unit having from 1 to 12 subunits;
s is 0 or 1;
L2 is a Linker Subunit having from 1 to 4 attachment sites for a Drug unit;
the wavy (˜) line indicates an attachment site for the Targeting unit, and the double wavy (≈) line indicates an attachment site for a Drug unit;
wherein at least one Polar unit is present within the Amino Acid unit, the Linker Subunit, or both, and wherein the Polar unit(s) is selected from Sugar units, PEG units, Carboxyl units, and combinations thereof.

In some embodiments, provided is a Linker having the following formula (I):

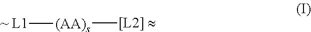

or a salt thereof, wherein:
L1 is a Stretcher unit having an attachment site for a Targeting unit;
AA is an Amino Acid unit having from 1 to 12 subunits;
s is 0 or 1;
L2 is a Linker Subunit having from 1 to 4 attachment sites for a Drug unit; the wavy (˜) line indicates an attachment site for the Targeting unit, and the double wavy (≈) line indicates an attachment site for a Drug unit;
wherein at least one Polar unit is present within the Amino Acid unit, the Linker Subunit, the Stretcher unit, or combinations thereof, and wherein the Polar unit(s) is selected from Sugar units, PEG units, Carboxyl units, and combinations thereof.

### Sugar Unit

In some embodiments, provided is a Linker intermediate or Linker, wherein the Sugar unit has the following formula:

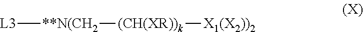

or a salt thereof, wherein:
   each X is independently selected from NH or 0;
   each R is independently selected from hydrogen, acetyl, a monosaccharide, a disaccharide, and a polysaccharide;
   each X₁ is independently selected from CH₂ and C(O);
   each X₂ is independently selected from H, OH and OR;
   k is 1 to 10; and
   L3 has the following general formula (XI):

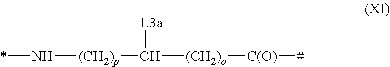

or a salt thereof, wherein:
   L3a is selected from C₁-C₁₀ alkylene and polyethylene glycol having from 1 to 24 ethylene glycol subunits;
   p and o are independently 0 to 2;
   each * and each # indicate an attachment site for another subunit of an Amino Acid unit (AA), a Linker subunit L2, or a Stretcher unit (L1); and
   L3a is covalently bound to the N atom marked with a ** in formula (X).

In some embodiments, the Linker intermediate or Linker comprises a Sugar unit having a formula selected from:

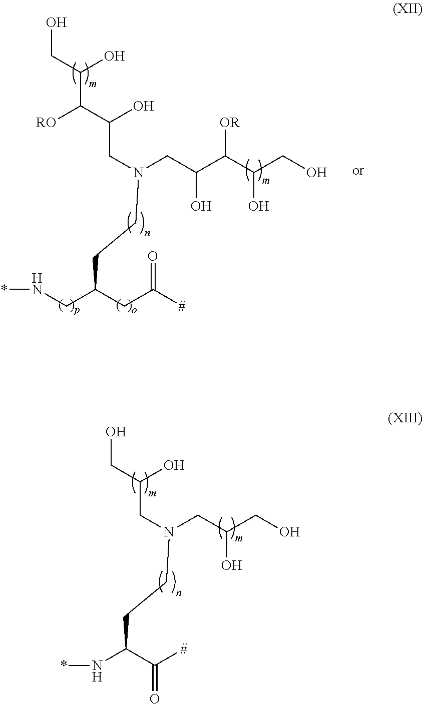

or a salt thereof, wherein:
   each R is independently selected from hydrogen, a monosaccharide, a disaccharide and a polysaccharide;
   p and o are independently 0 to 2;
   m is 1-8;
   n is 0 to 4; and
   each * and each # indicate an attachment site for another subunit of the Amino Acid unit (AA), the Linker subunit L2, or the Stretcher unit (L1).

### PEG Unit

In some embodiments, Linker intermediate or Linker comprises a PEG unit having a formula selected from:

(a)

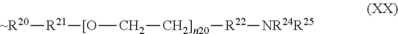

or a salt thereof, wherein:
   R²⁰ is a functional group for attachment to a subunit of the Amino Acid unit or a portion of the Linker Subunit L2;
   R²¹ and R²² are each, independently, optional C₁-C₃ alkylene;
   R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); or —NR²⁴R²⁵ together from a C₃-C₈ heterocycle; the wavy line (˜) indicates the attachment site to R²⁰; and
   n20 is 1 to 26;
   or
(b)

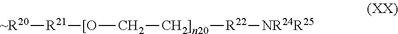

or a salt thereof, wherein:
R²⁰ is a functional group for attachment to a subunit of the Amino Acid unit or a portion of the Linker Subunit L2;
R²¹ and R²² are each, independently, optional C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is a polyethylene glycol, optionally having 1 to 24 ethylene glycol subunits;
the wavy line (˜) indicates the attachment site to R²⁰; and n20 is 1 to 26;
or

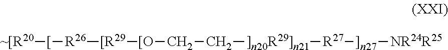

or a salt thereof, wherein:
R²⁰ is a functional group for attachment to a subunit of an Amino Acid unit and/or a portion of a Linker Subunit L2;
R²⁶ and R²⁷ are each optional and are, independently, selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle;
each R²⁹ is optional and independently selected from —C(O)—, —NH—, —C(O)—C₁-C₆ alkenylene-, —NH—C₁-C₆ alkenylene-, —C₁-C₆ alkenylene-NH—, —C₁-C₆ alkenylene-C(O)—, —NH(CO)NH—, and triazole;
the wavy line (˜) indicates the attachment site to R²⁰
n20 is 1 to 26;
n21 is 1 to 4; and
n27 is 1 to 4.

In some embodiments, provided is a Linker intermediate or Linker, wherein both R²⁴ and R²⁵ of the PEG unit are not H. In some embodiments, provided is a Linker intermediate or Linker, wherein R²⁴ and R²⁵ of the PEG unit are each independently selected from H and polyhydroxyl group, provided that R²⁴ and R²⁵ are not both H.

In some embodiments, provided is a Linker intermediate or Linker, wherein the polyhydroxyl group is a linear monosaccharide, optionally selected from a C₆ or C₅ sugar, sugar acid or amino sugar. In some embodiments, provided is a Linker intermediate or Linker, wherein:
the C₆ or C₅ sugar is selected from glucose, ribose, galactose, mannose, arabinose, 2-deoxyglucose, glyceraldehyde, erythrose, threose, xylose, lyxose, allose, altrose, gulose, idose talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

In some embodiments, provided is a Linker intermediate or Linker, wherein the PEG unit is selected from the following, or a salt thereof:

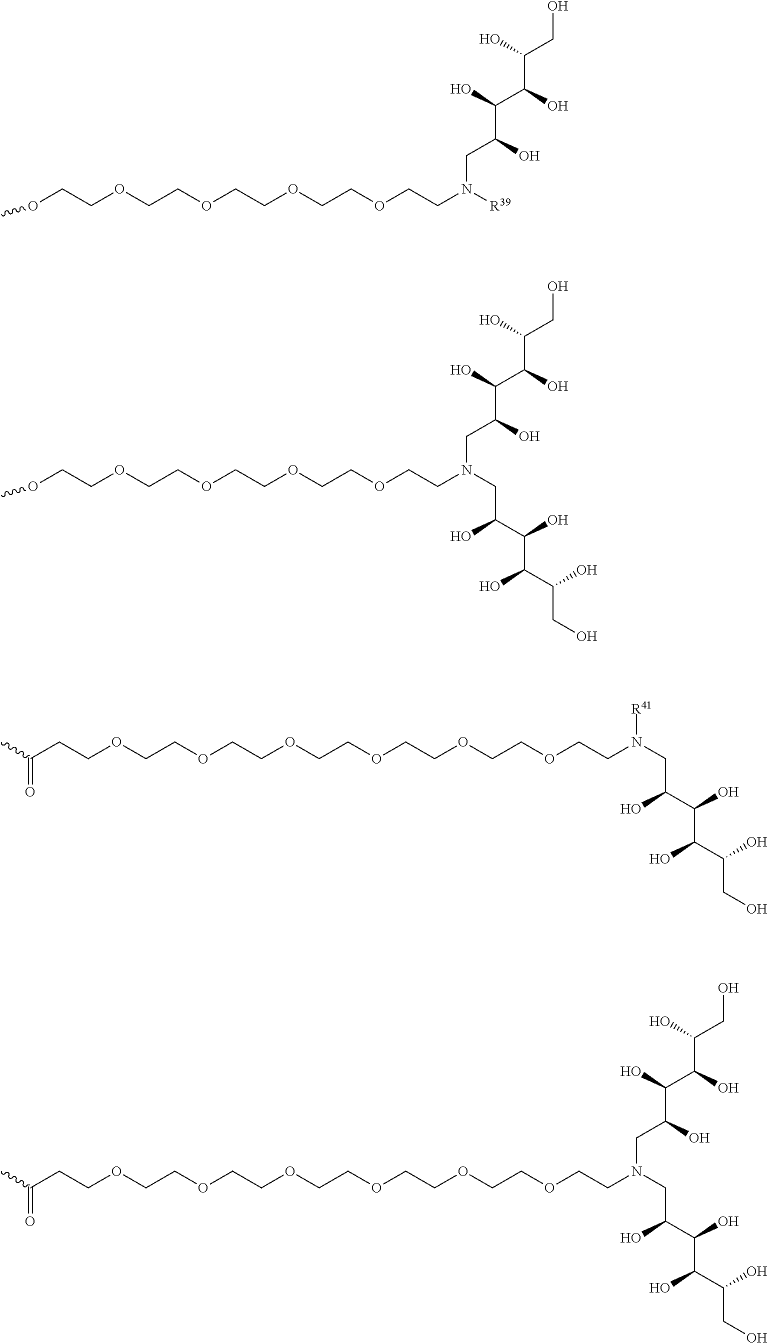

wherein R³⁹ is selected from H, a linear monosaccharide and polyethylene glycol, optionally having from 1 to 24 ethylene glycol subunits; and the wavy line at the left side indicates the attachment site to the subunit of the Amino Acid unit or the portion of the Linker subunit.

In some embodiments, provided is a Linker intermediate or Linker, wherein one of R²⁴ and R²⁵ of the PEG unit is a linear monosaccharide and the other is a cyclic monosaccharide.

In some embodiments, provided is a Linker intermediate or Linker, wherein the PEG unit is selected from the following, or a salt thereof:

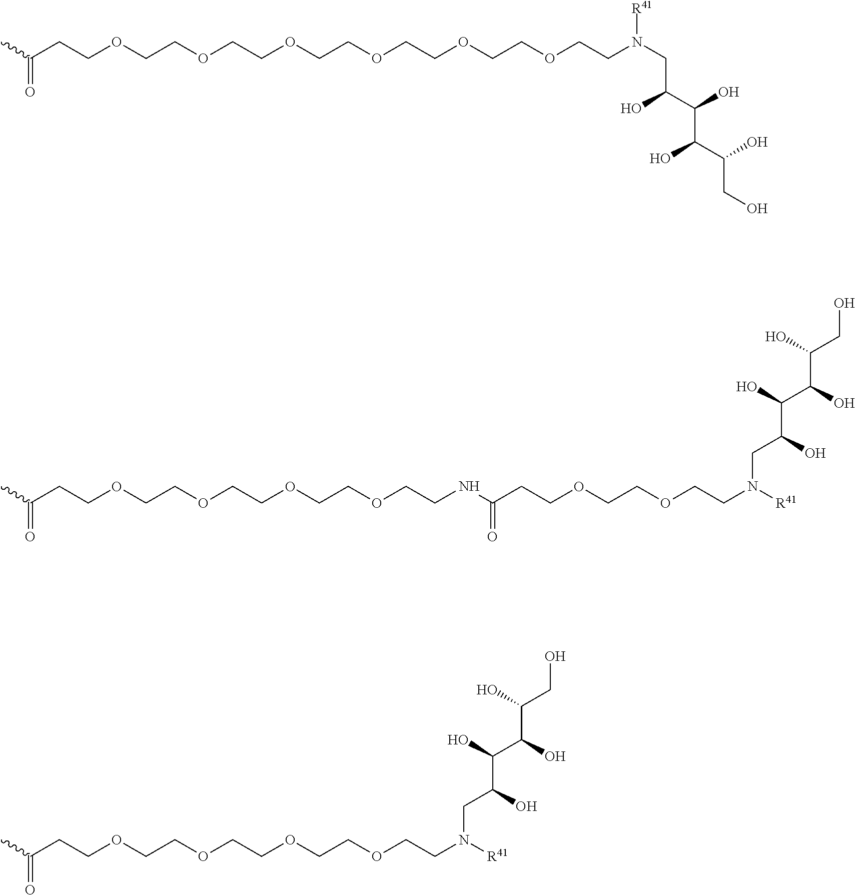

wherein R⁴¹ is a cyclic monosaccharide; and the wavy line at the left side indicates the attachment site to the subunit of the Amino Acid unit or the portion of the Linker subunit.

In some embodiments, provided is a Linker intermediate or Linker, wherein R²⁴ and R²⁵ of the PEG unit are independently selected from cyclic monosaccharides, disaccharides and polysaccharides. In some embodiments, provided is a Linker intermediate or Linker, wherein the PEG unit is selected from the following, or a salt thereof:

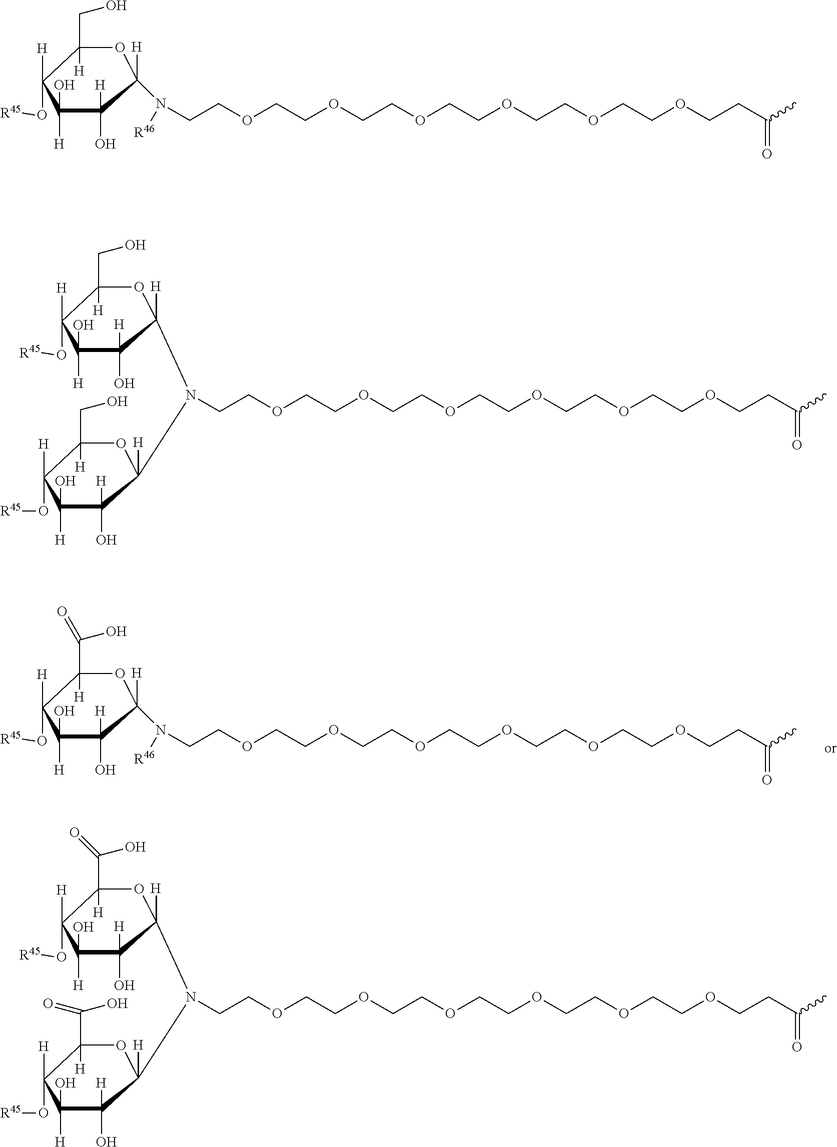

wherein each R⁴⁵ is selected from H and a monosaccharide, a disaccharide, or a polysaccharide; and R⁴⁶ is selected from a cyclic monosaccharide, disaccharide, or polysaccharide; and the wavy line at the right side indicates the attachment site to the subunit of the Amino Acid unit or the portion of the Linker subunit.

In some embodiments, provided is a Linker intermediate or Linker, wherein R²⁴ and R²⁵ of the PEG unit are independently selected from a linear monosaccharide and a substituted linear monosaccharide, wherein the substituted linear monosaccharide is substituted with a monosaccharide, a disaccharide or a polysaccharide. In some embodiments, provided is a Linker intermediate or Linker, wherein the PEG unit is selected from the following, or a salt thereof:

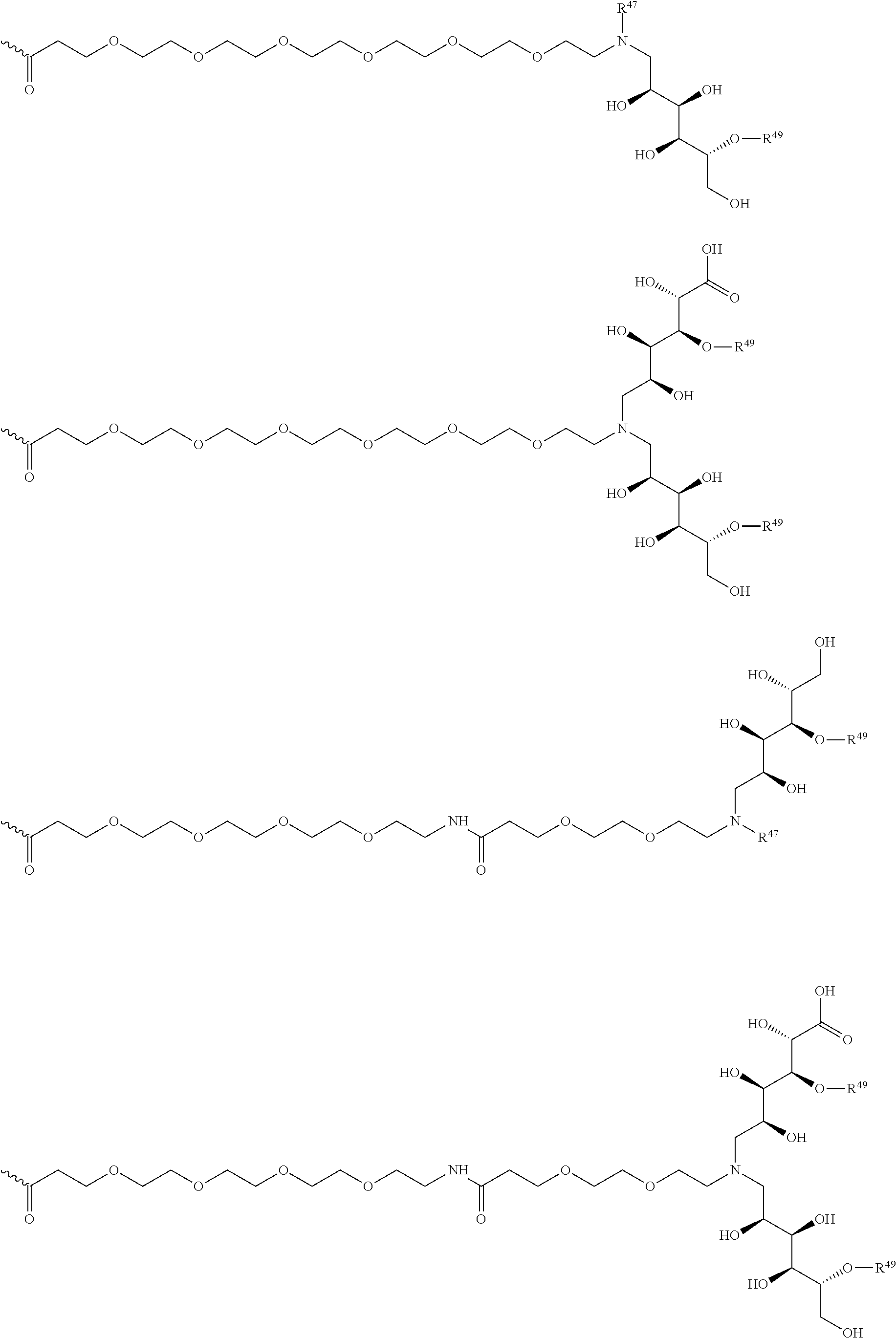

wherein R⁴⁷ is a linear monosaccharide; and each R⁴⁹ is selected from a monosaccharide, a disaccharide and a polysaccharide; and the wavy line at the left side indicates the attachment site to the subunit of the Amino Acid unit or the portion of the Linker subunit.

In some embodiments, provided is a Linker intermediate or Linker, wherein R²⁴ and R²⁵ of the PEG unit are independently selected from a linear monosaccharide and a substituted monosaccharide, wherein the substituted linear monosaccharide is substituted with one or more substituents selected from alkyl, O-alkyl, aryl, O-aryl, carboxyl, ester, or amide, and optionally further substituted with a monosaccharide, disaccharide or a polysaccharide. In some embodiments, provided is a Linker intermediate or Linker, wherein the PEG unit is selected from the following, or a salt thereof:

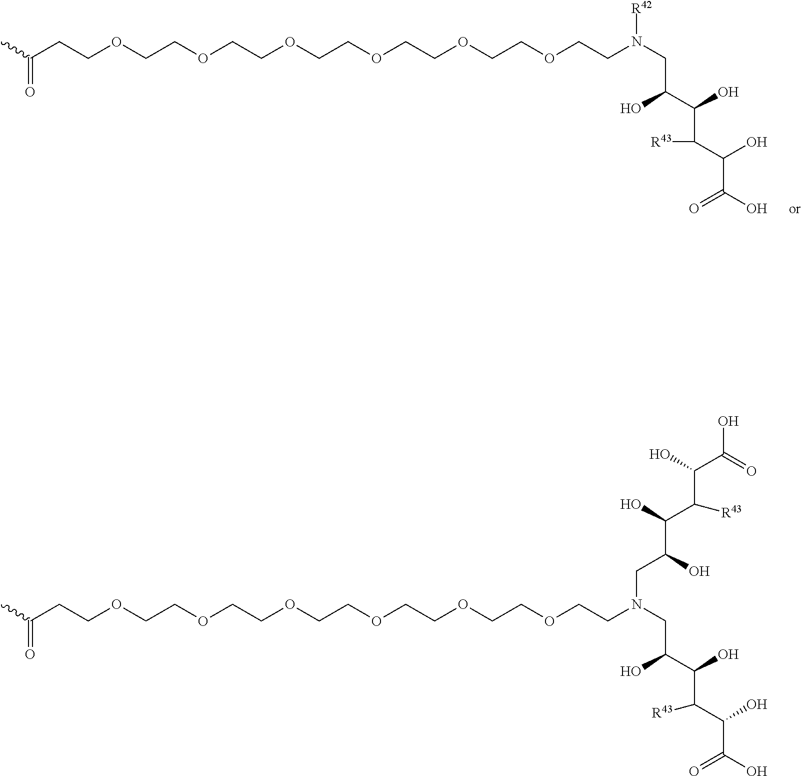

wherein each R⁴² is independently selected from a linear monosaccharide and a substituted linear monosaccharide; each R⁴³ is independently selected from alkyl, O-alkyl, aryl, O-aryl, carboxyl, ester, and amide; and the wavy line at the left side indicates the attachment site to the subunit of the Amino Acid unit or the portion of the Linker subunit.

In some embodiments, provided is a Linker intermediate or Linker, wherein one of R²⁴ and R²⁵ of the PEG unit is a —C(O)-polyhydroxyl group or substituted —C(O)-polyhydroxyl group, and the other of R²⁴ and R²⁵ is a H, —C(O)-polyhydroxyl group, substituted —C(O)— polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, alkyl, —O-alkyl, aryl, carboxyl, ester, or amide. In some embodiments, provided is a Linker intermediate or Linker, wherein the PEG unit is selected from the following, or a salt thereof:

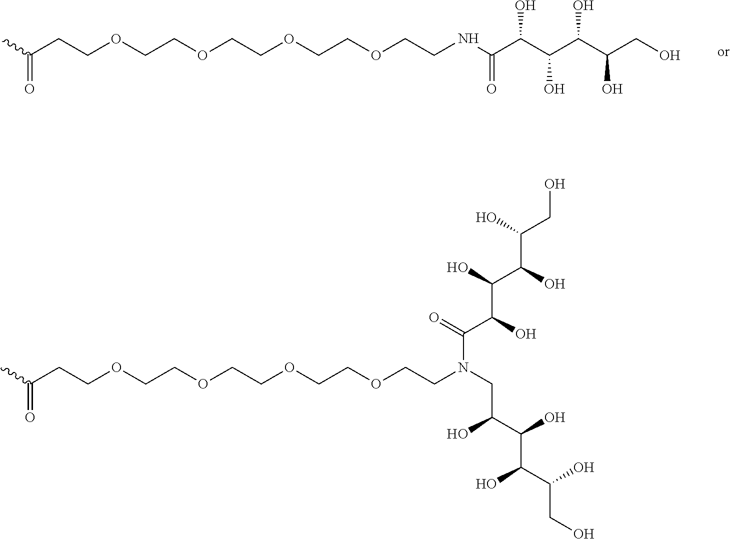

wherein the wavy line at the left side indicates the attachment site to the subunit of the Amino Acid unit or the portion of the Linker subunit.

In some embodiments, provided is a Linker intermediate or Linker, wherein R²⁴ and R²⁵ of the PEG unit are independently selected from a H, substituted —C₁-C₈ alkyl, substituted —C₁-C₄ alkyl or substituted —C₁-C₃ alkyl; provided that both R²⁴ and R²⁵ are not H; wherein substituted —C₁-C₈ alkyl, —C₁-C₄ alkyl and —C₁-C₃ alkyl are substituted with hydroxyl and/or carboxyl. In some embodiments, provided is a Linker intermediate or Linker, wherein the PEG unit is selected from the following, or a salt thereof:

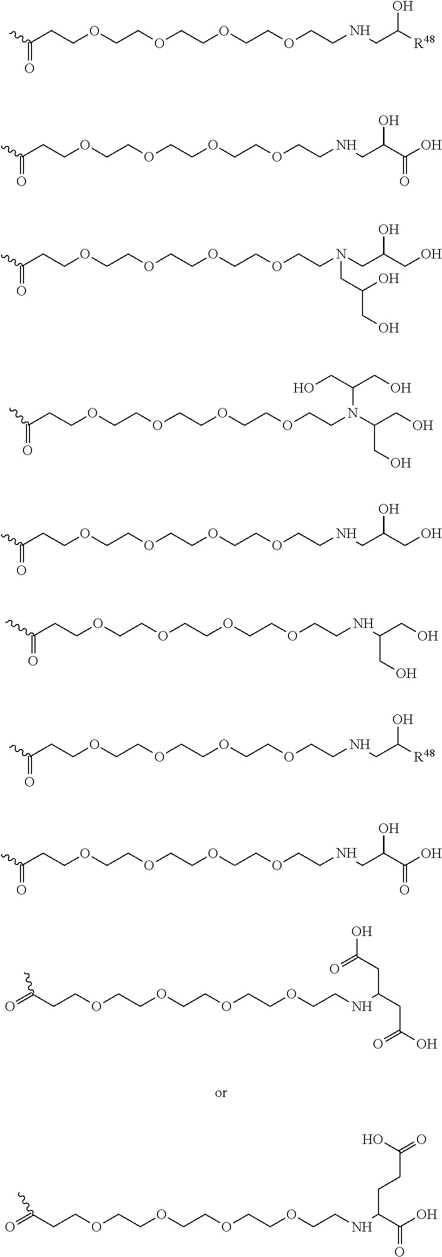

wherein R⁴⁸ is selected from H, OH, CH₂OH, COOH or —C₁-C₆ alkyl substituted with hydroxyl or carboxyl; and the wavy line at the left side indicates the attachment site to the subunit of the Amino Acid unit or the portion of the Linker subunit.

In some embodiments, provided is a Linker intermediate or Linker, wherein one of R²⁴ and R²⁵ of the PEG unit is selected from H, substituted —C(O)—C₁-C₈ alkyl, substituted —C(O)—C₁-C₄ alkyl, and substituted —C(O)—C₁-C₃ alkyl and the other of R²⁴ and R²⁵ is selected from substituted —C(O)—C₁-C₈ alkyl, substituted —C(O)—C₁-C₄ alkyl, substituted —C(O)—C₁-C₃ alkyl, substituted —C₁-C₈ alkyl, substituted —C₁-C₄ alkyl, and substituted —C₁-C₃ alkyl, wherein substituted —C(O)—C₁-C₈ alkyl, substituted —C(O)—C₁-C₄ alkyl, substituted —C(O)—C₁-C₃ alkyl, substituted —C₁-C₈ alkyl, —C₁-C₄ alkyl and —C₁-C₃ alkyl are substituted with hydroxyl and/or carboxyl. In some embodiments, provided is a Linker intermediate or Linker, wherein the PEG unit is selected from the following, or a salt thereof:

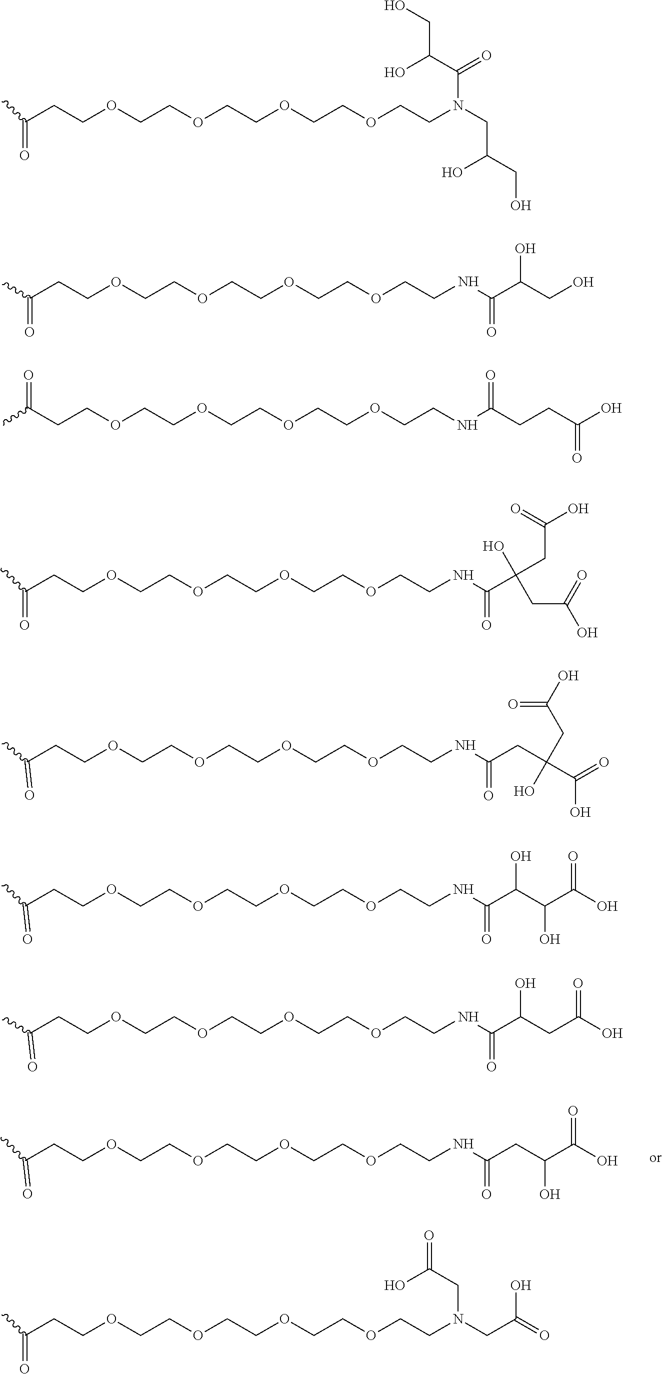

wherein the wavy line at the left side indicates the attachment site to the subunit of the Amino Acid unit or the portion of the Linker subunit.

In some embodiments, provided is a Linker intermediate or Linker, wherein R²⁴ and R²⁵ of the PEG unit are selected from H and optionally substituted aryl; provided that both R²⁴ and R²⁵ are not H, wherein the optional substituents are as defined herein, for example in some embodiments the optional subsituten is halo, such as F, Cl, or Br. In some embodiments, provided is a Linker intermediate or Linker wherein the PEG unit is selected from the following, or a salt thereof:

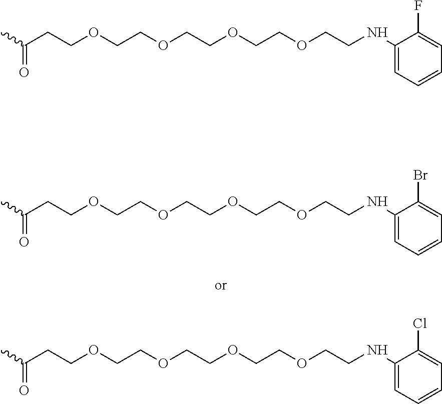

wherein the wavy line at the left side indicates the attachment site to the subunit of the Amino Acid unit or the portion of the Linker subunit.

In some embodiments, provided is a Linker intermediate or Linker, wherein R²⁴ and R²⁵ together form an optionally substituted C₃-C₈ heterocycle or heteroaryl, wherein in some embodiments the C₃-C₈ heterocycle or heteroaryl is unsubstituted. In some embodiments, provided is a Linker intermediate or Linker wherein the PEG unit is:

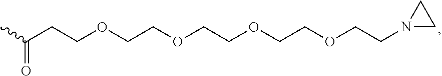

or a salt thereof.

In some embodiments, provided is a Linker intermediate or Linker, wherein R²⁴ and R²⁵ of the PEG unit are independently selected from H and a chelator, wherein the chelator is optionally attached to the nitrogen of —NR²⁴R²⁵ by an alkylene, arylene, carbocyclo, heteroarylene or heterocarbocylo; provided that both R²⁴ and R²⁵ are not H. In some embodiments, provided is a Linker intermediate or Linker, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine. In some embodiments, provided is a Linker intermediate or Linker, wherein the PEG unit is selected from the following, or a salt thereof:

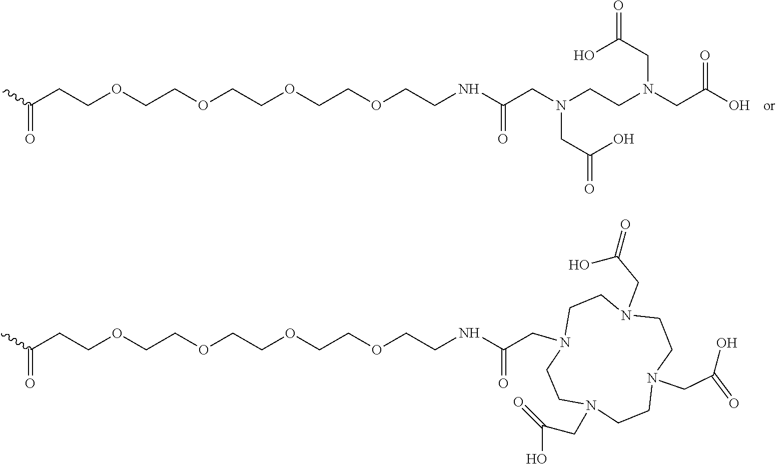

wherein the wavy line at the left side indicates the attachment site to the subunit of the Amino Acid unit or the portion of the Linker subunit.

In some embodiments, provided is a Linker intermediate or Linker, wherein each monosaccharide of a Sugar unit or a PEG unit is independently selected from:
a C₅ or C₆ sugar selected from glucose, ribose, galactose, mannose, arabinose, 2-deoxyglucose, glyceraldehyde, erythrose, threose, xylose, lyxose, allose, altrose, gulose, idose talose, aldose, ketose, glucosamine, N-acetyl glucosamine, galactosamine, and N-acetyl galactosamine;
a sugar acid selected from gluconic acid, aldonic acid, uronic acid and ulosonic acid; or
an amino sugar is selected from glucosamine, N-acetyl glucosamine, galactosamine, and N-acetyl galactosamine.

In some embodiments, provided is a Linker intermediate or Linker, wherein R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof.

In some embodiments, provided is a Linker intermediate or Linker, wherein R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

In some embodiments, provided is a Linker intermediate or Linker, wherein the PEG unit has the formula selected from the following:
(a)

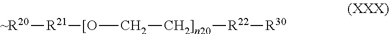

or a salt thereof, wherein:
   R²⁰ is a functional group for attachment to a subunit of the Amino Acid unit (if present) and/or a portion of Linker Subunit L2;
   R²¹ and R²² are each optional and, if present, are independently, C₁-C₃ alkylene groups;
   R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
   the wavy line (˜) indicates the attachment site to R²⁰; and n20 is 1 to 26;
(b)

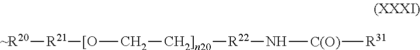

or a salt thereof, wherein:
   R²⁰ is a functional group for attachment to a subunit of the Amino Acid unit (if present) or a portion of the Linker Subunit L2;
   R²¹ and R²² are each, independently, optional C₁-C₃ alkylene groups;
   R³¹ is a branched polyethylene glycol chain, each branch having 1 to 26 ethylene glycol subunits and each branch having an R³⁵ at its terminus;
   R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
   the wavy line (˜) indicates the attachment site to R²⁰; and n20 is 1 to 26;
(c)

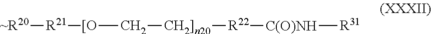

or a salt thereof, wherein:
   R²⁰ is a functional group for attachment to a subunit of the Amino Acid unit (if present) or a portion of the Linker Subunit L2;
   R²¹ and R²² are each optional and are, independently, C₁-C₃ alkylene groups;
   R³¹ is a branched polyethylene glycol chain, each branch, independently, having 1 to 26 ethylene glycol subunits and each branch having an R³⁵ at its terminus;
   R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl;
   the wavy line (˜) indicates the attachment site to R²⁰; and n20 is 1 to 26; and
(d)

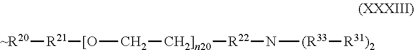

or a salt thereof, wherein:
   R²⁰ is a functional group for attachment to a subunit of the Amino Acid unit (if present) or a portion of the Linker Subunit L2;
   R²¹ and R²² are each optional and are C₁-C₃ alkylene groups;
   R³¹ is a branched polyethylene glycol chain, each branch having 1 to 26 ethylene glycol subunits and each branch having an R³⁵ at its terminus;
   R³³ is C₁-C₃ alkylene, C₁-C₃ alkylene-C(O), —C(O)—C₁-C₃ alkylene, or —C(O)—C₁-C₃ alkylene-C(O);
   R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
   the wavy line (˜) indicates the attachment site to R²⁰; and n20 is 1 to 26.

In some embodiments, provided is a Linker intermediate or Linker, wherein the PEG unit has a formula selected from the following, or a salt thereof:

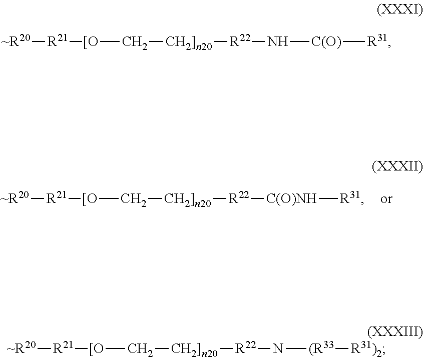

wherein R²⁰ is a functional group for attachment to a subunit of the Amino Acid unit (if present) or a portion of the Linker Subunit L2; R²¹ and R²² are each optional and are C₁-C₃ alkylene groups; R³¹ is a branched polyethylene glycol chain, each branch having 1 to 26 ethylene glycol subunits and each branch having an R³⁵ at its terminus; R³³ is C₁-C₃ alkylene, —C₁-C₃ alkylene-C(O), —C(O)—C₁-C₃ alkylene or —C(O)—C₁-C₃ alkylene-C(O); R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 1 to 26. In some embodiments, provided is a Linker intermediate or Linker, wherein the PEG unit is selected from the following:

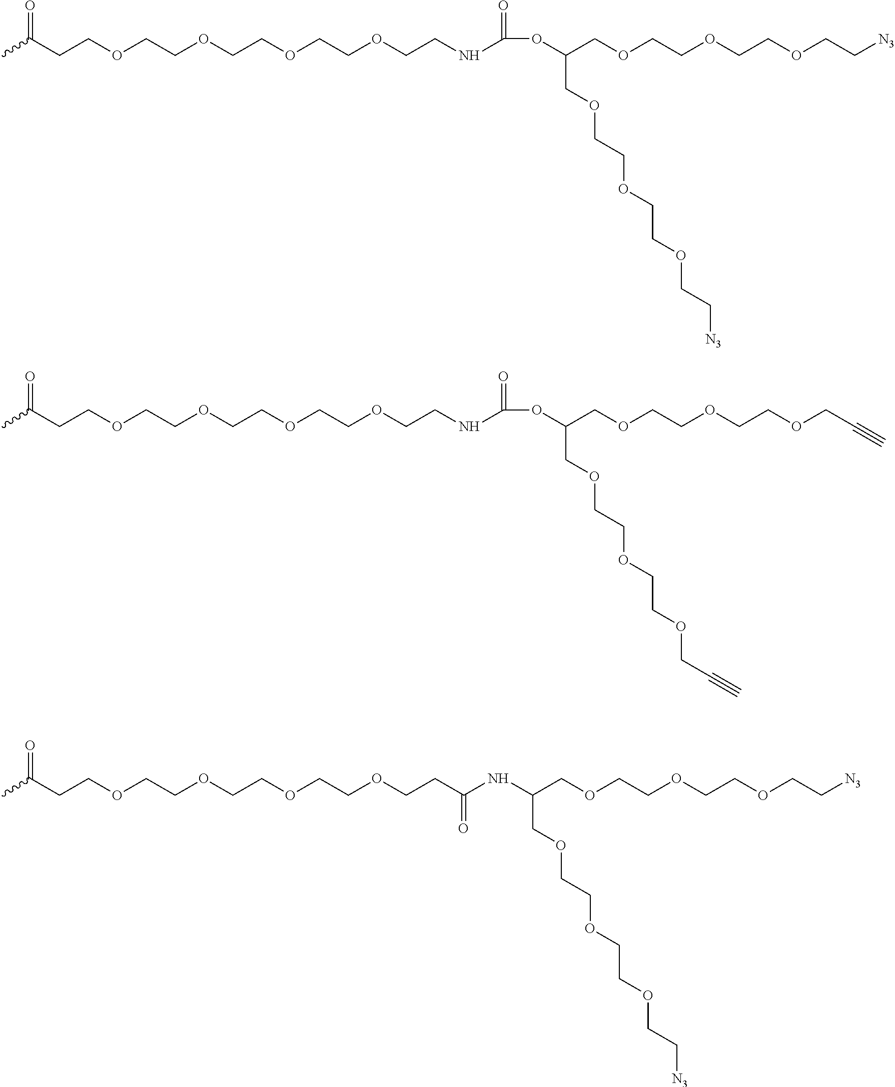

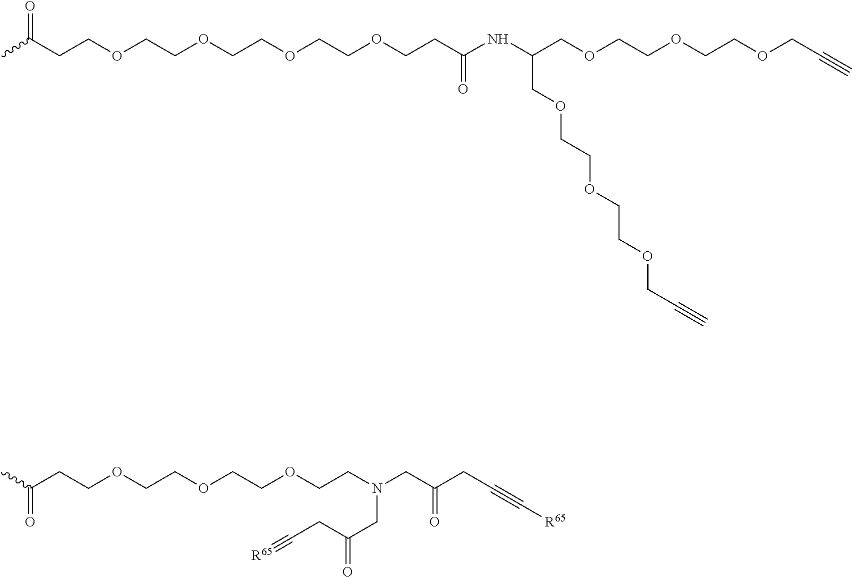

wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line at the left side indicates the attachment site to the subunit of the Amino Acid unit or the portion of the Linker subunit.

In some embodiments, provided is a Linker intermediate or Linker, wherein R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof.

In some embodiments, provided is a Linker intermediate or Linker, wherein R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

In some embodiments, provided is a Linker intermediate or Linker, comprising a PEG unit having a formula selected from:

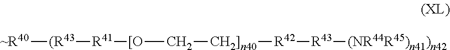

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₆ alkylene;
each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)— polyhydroxyl group, wherein optional substituents are selected from sulfate, phosphate, alkyl sulfate, and alkyl phosphate;
the wavy line (˜) indicates the attachment site to R⁴⁰;
n40 is 1 to 26;
n41 is 1 to 6; and
n42 is 1 to 6.

In some embodiments, provided is a Linker intermediate or Linker, comprising a PEG unit having a formula selected from:

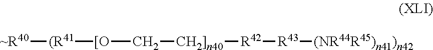

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₆ alkylene;
R⁴³ is absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)— polyhydroxyl group, wherein optional substituents are selected from sulfate, phosphate, alkyl sulfate, and alkyl phosphate;
the wavy line (˜) indicates the attachment site to R⁴⁰;
n40 is 1 to 26;
n41 is 1 to 6; and
n42 is 1 to 6.

In some embodiments, provided is a Linker intermediate or Linker, comprising a PEG unit having a formula selected from:

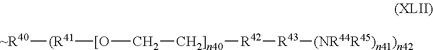

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₃ alkylene;
R⁴³ is absent or is selected from selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)— polyhydroxyl group, wherein optional substituents are selected from sulfate, phosphate, alkyl sulfate, and alkyl phosphate;
the wavy line (˜) indicates the attachment site to R⁴⁰;
n40 is 1 to 16;
n41 is 1 to 4; and
n42 is 1 to 4.

In some embodiments, provided is a Linker intermediate or Linker, wherein R⁴⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

In some embodiments, provided is a Linker intermediate or Linker, wherein R⁴⁰ has one of the following structures:

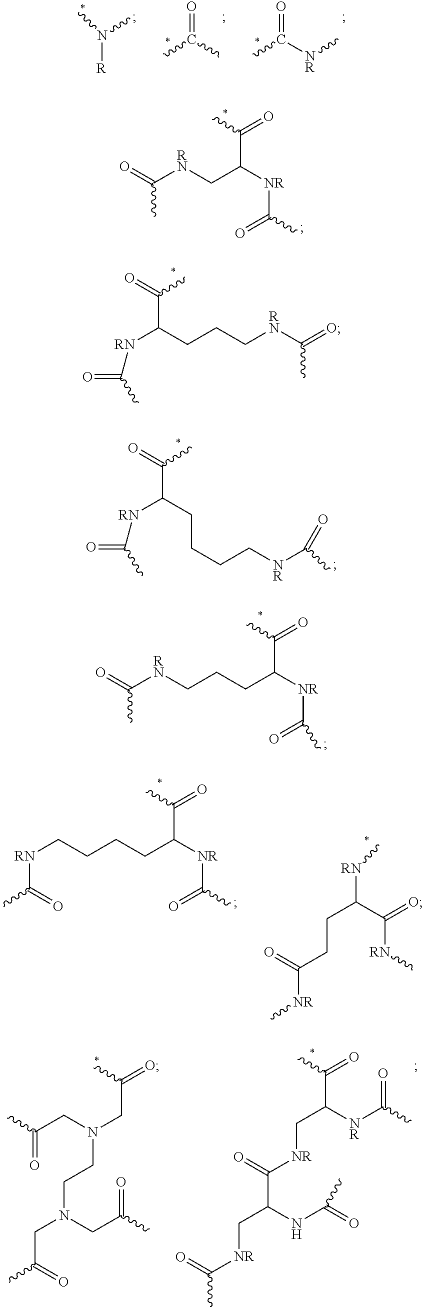

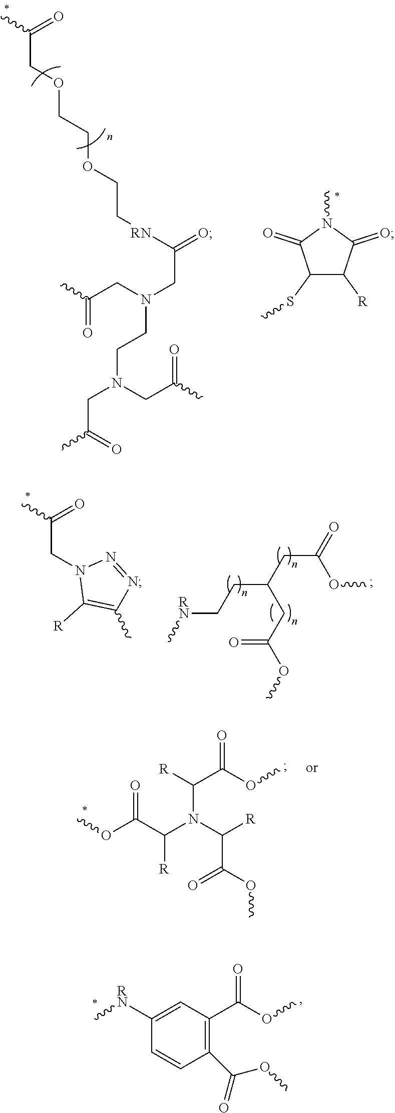

wherein R═H or C₁₋₆alkyl; and
n=0 to 12
or a stereoisomer thereof, wherein the (*) indicates the attachment site of R⁴⁰ to a subunit of the Amino Acid unit or a portion of the Linker Subunit L2 and the ( [symbol] ) indicates the attachment site of R⁴⁰ to the remainder of the PEG unit.

In some embodiments, provided is a Linker intermediate or Linker, wherein R⁴⁰ has one of the following structures:

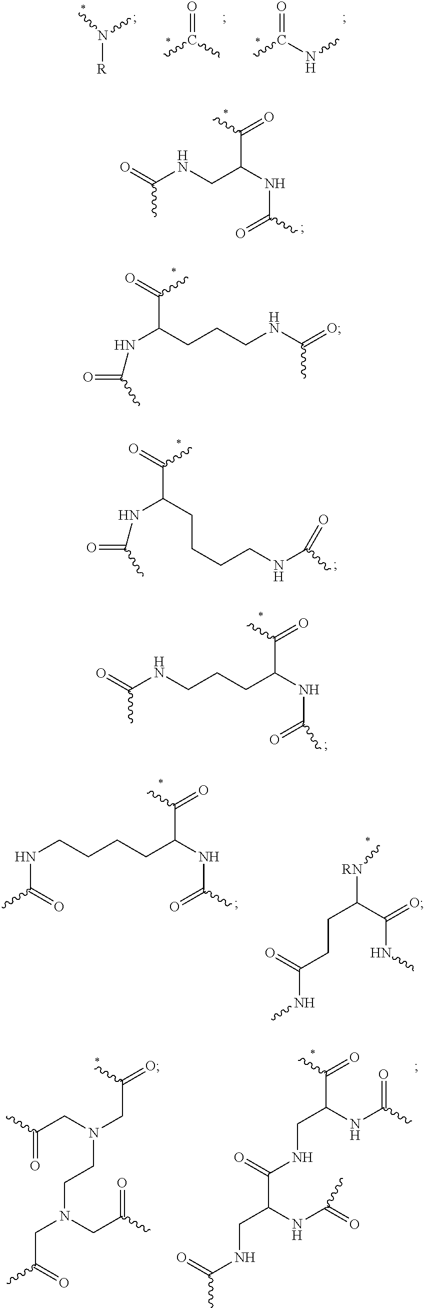

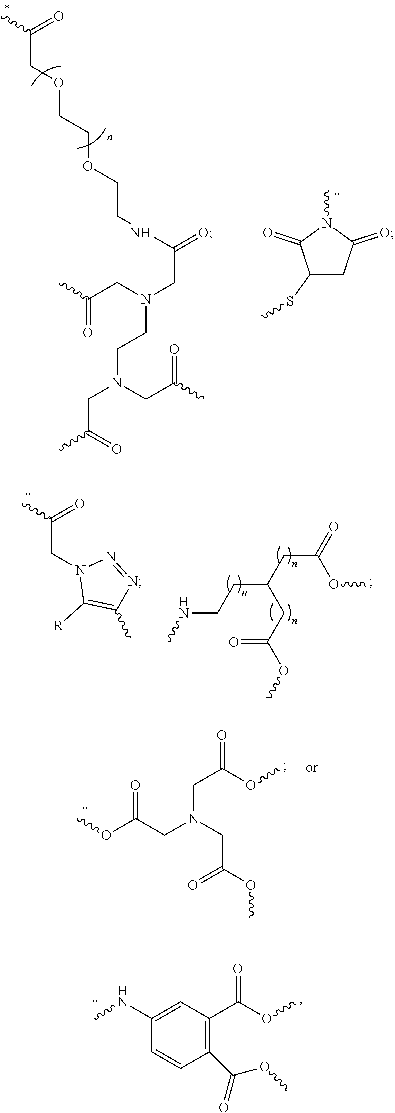

wherein n=0 to 12
or a stereoisomer thereof, wherein the (*) indicates the attachment site of R⁴⁰ to a subunit of the Amino Acid unit or a portion of the Linker Subunit L2 and the ( [symbol] ) indicates the attachment site of R⁴⁰ to the remainder of the PEG unit.

In some embodiments, provided is a Linker intermediate or Linker, wherein R⁴³—(NR⁴⁴R⁴⁵)ₙ₄₁, when R⁴³ is present, has one of the following structures:

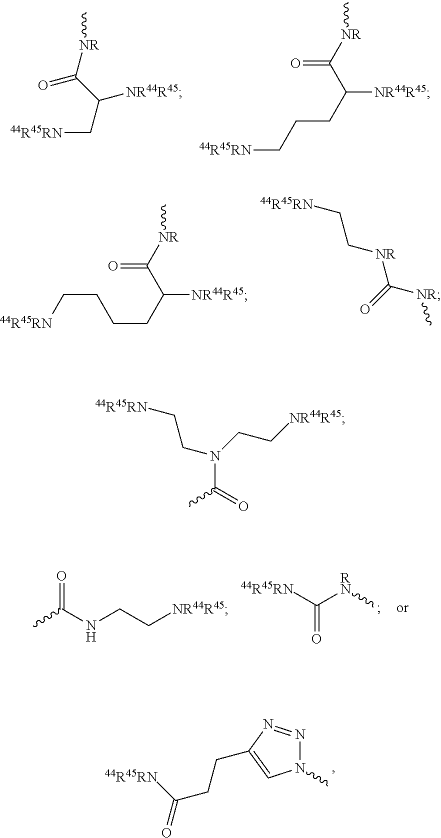

wherein R═H, C₁₋₆alkyl, polyhydroxyl, or substituted polyhydroxyl
or a stereoisomer thereof, wherein the ( [symbol] ) indicates the attachment site of R⁴³ to the remainder of the PEG unit.

In some embodiments, provided is a Linker intermediate or Linker, wherein R⁴³—(NR⁴⁴R⁴⁵)ₙ₄₁, when R⁴³ is present, has one of the following structures:

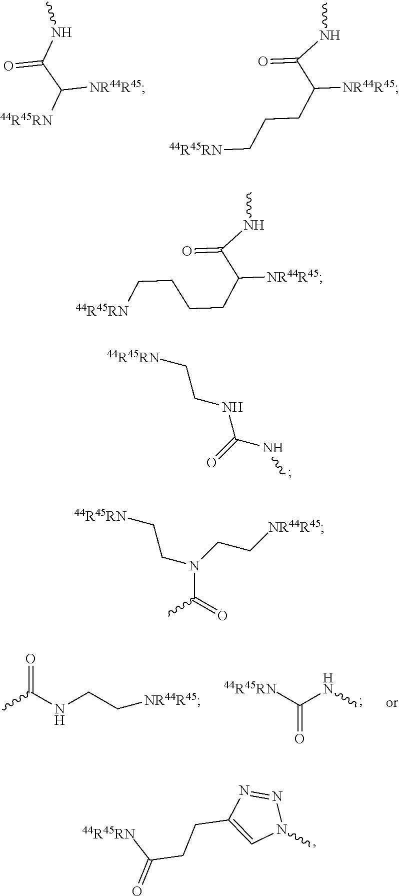

or a stereoisomer thereof, wherein the ( [symbol] ) indicates the attachment site of R⁴³ to the remainder of the PEG unit.

In some embodiments, provided is a Linker intermediate or Linker, wherein —NR⁴⁴R⁴⁵ has one of the following structures:

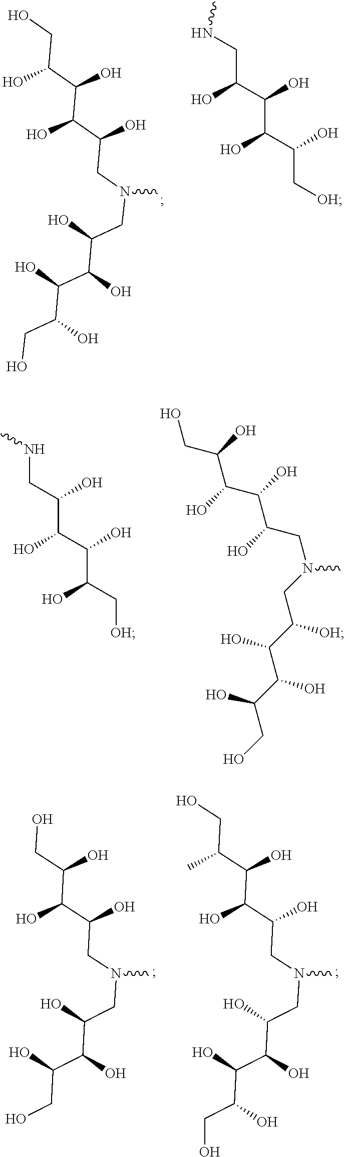

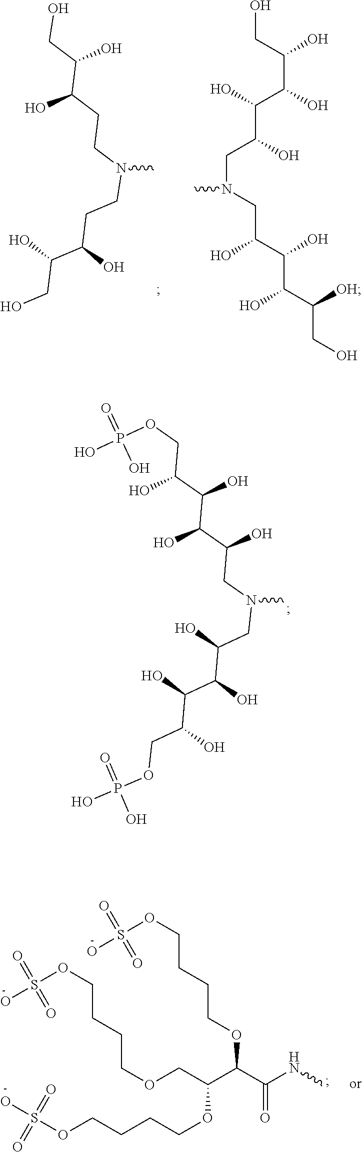

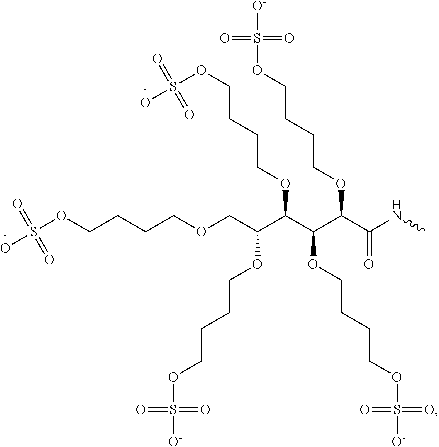

or a stereoisomer thereof, wherein the ( [symbol] ) indicates the attachment site of —NR⁴⁴R⁴⁵ to the remainder of the PEG unit.

In some embodiments, provided is a Linker intermediate or Linker, wherein the PEG unit has one of the following structures prior to attachment to the Amino Acid unit or to a portion of the Linker Subunit L2:

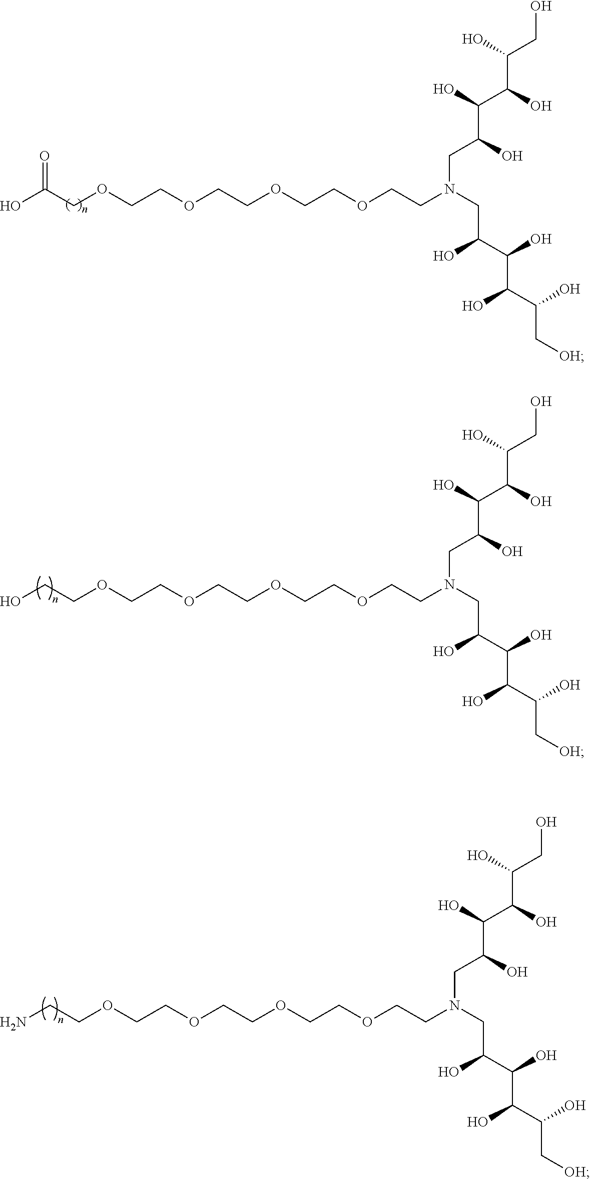

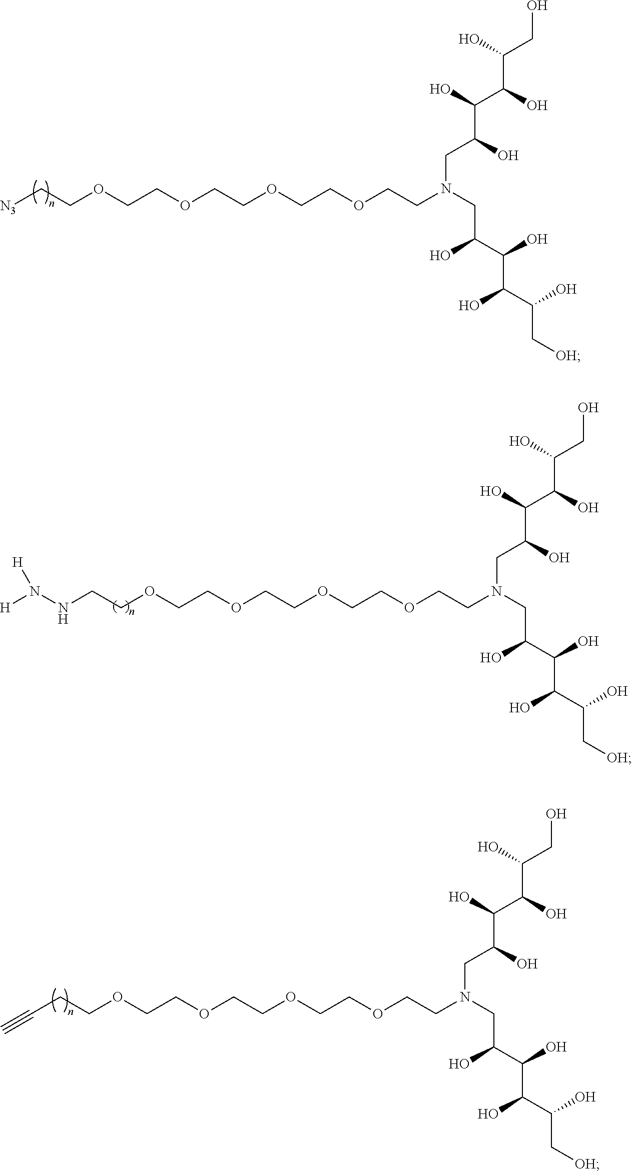

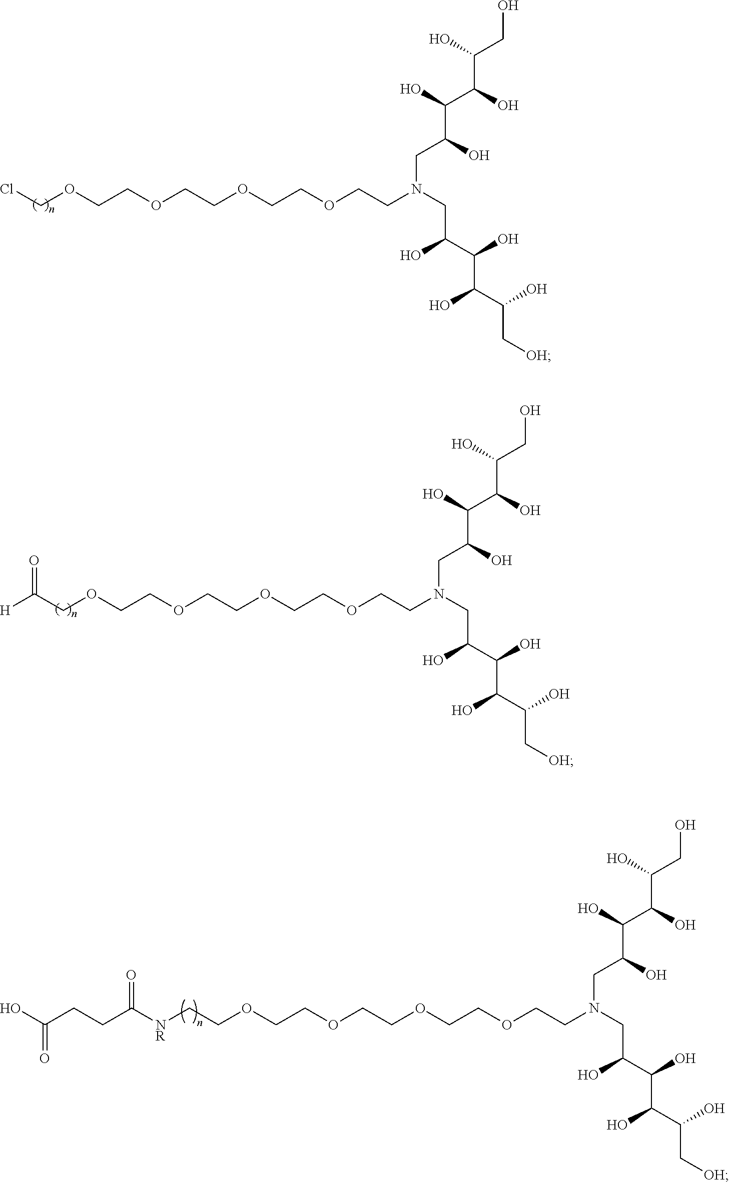

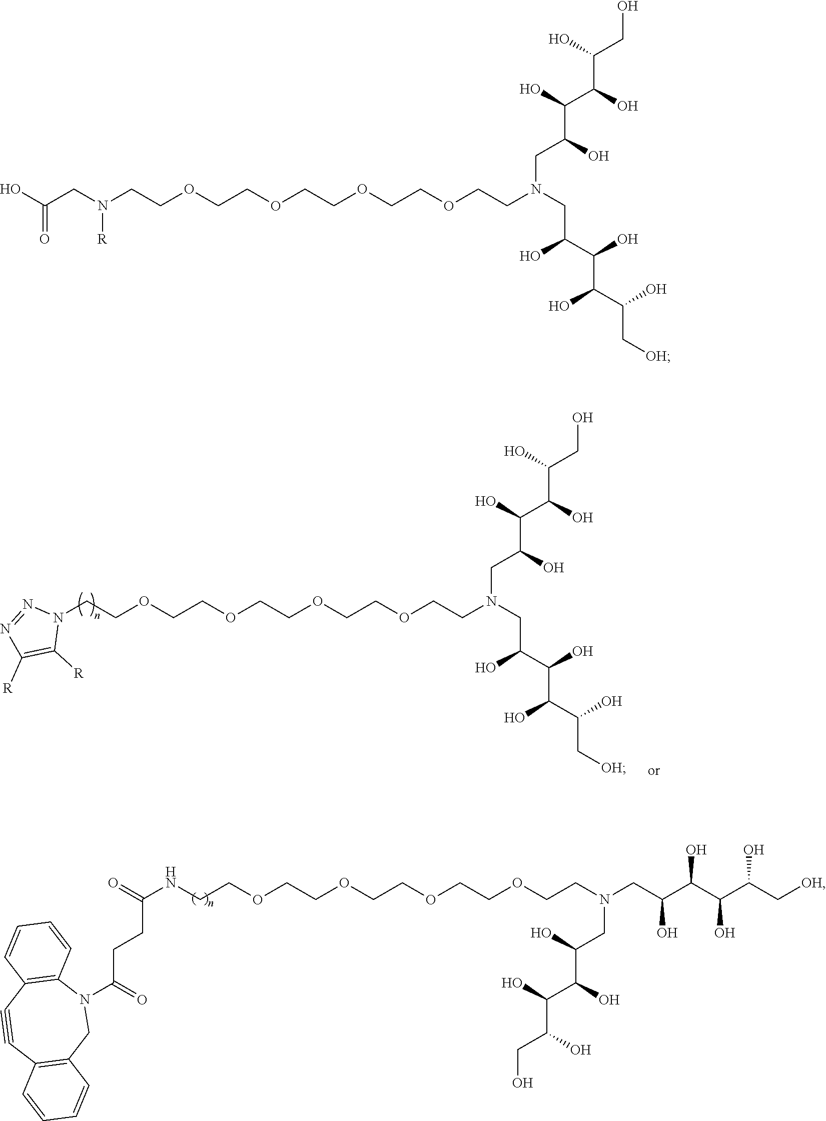

wherein R is H or alkyl, and n is 1 to 12.

In some embodiments, provided is a Linker intermediate or Linker, comprising a PEG unit having a formula selected from:

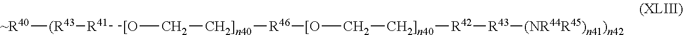

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₆ alkylene;
each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)— polyhydroxyl group, wherein optional substituents are selected from sulfate, phosphate, alkyl sulfate, and alkyl phosphate;
R⁴⁶ is selected from amino, amino-alkyl-amino, or —NH—C(O)—NH—S(O)₂—NH—; the wavy line (˜) indicates the attachment site to R⁴⁰;
n40 is 1 to 26;
n41 is 1 to 6; and
n42 is 1 to 6.

In some embodiments, provided is a Linker intermediate or Linker, wherein the PEG unit has one of the following structures prior to attachment to the Amino Acid unit or to a portion of the Linker Subunit L2:

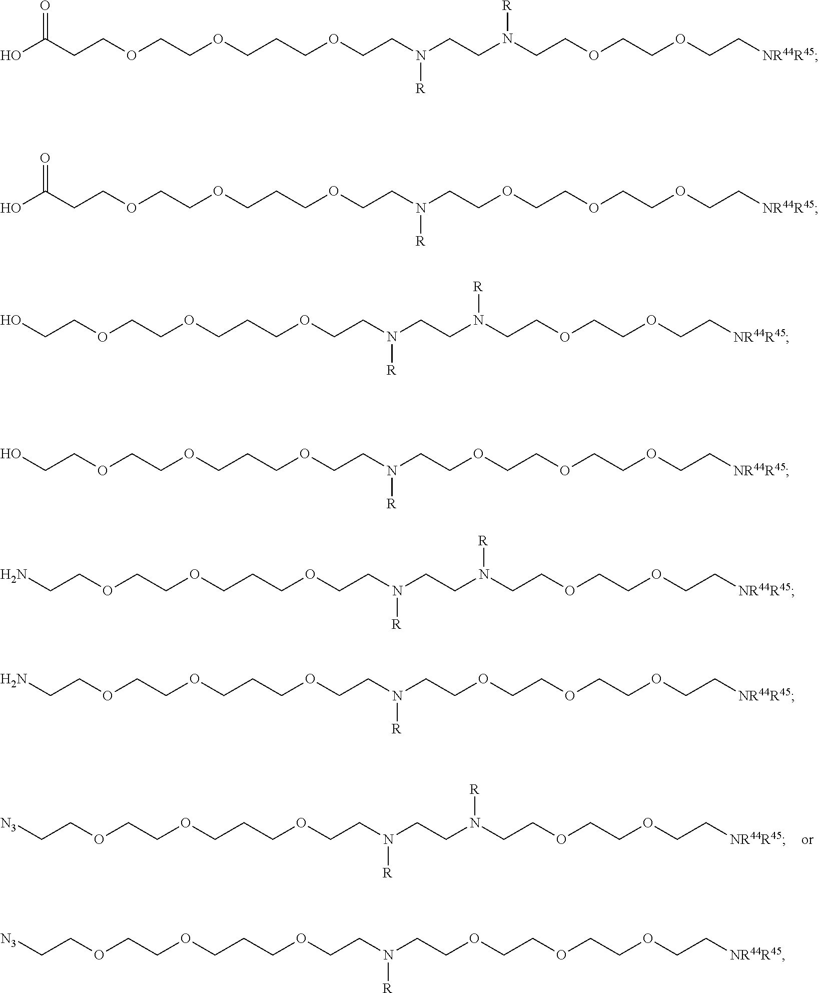

wherein R is H or alkyl, and n is 1 to 12.

In some embodiments, provided is a Linker intermediate or Linker, comprising a PEG unit having a formula selected from:

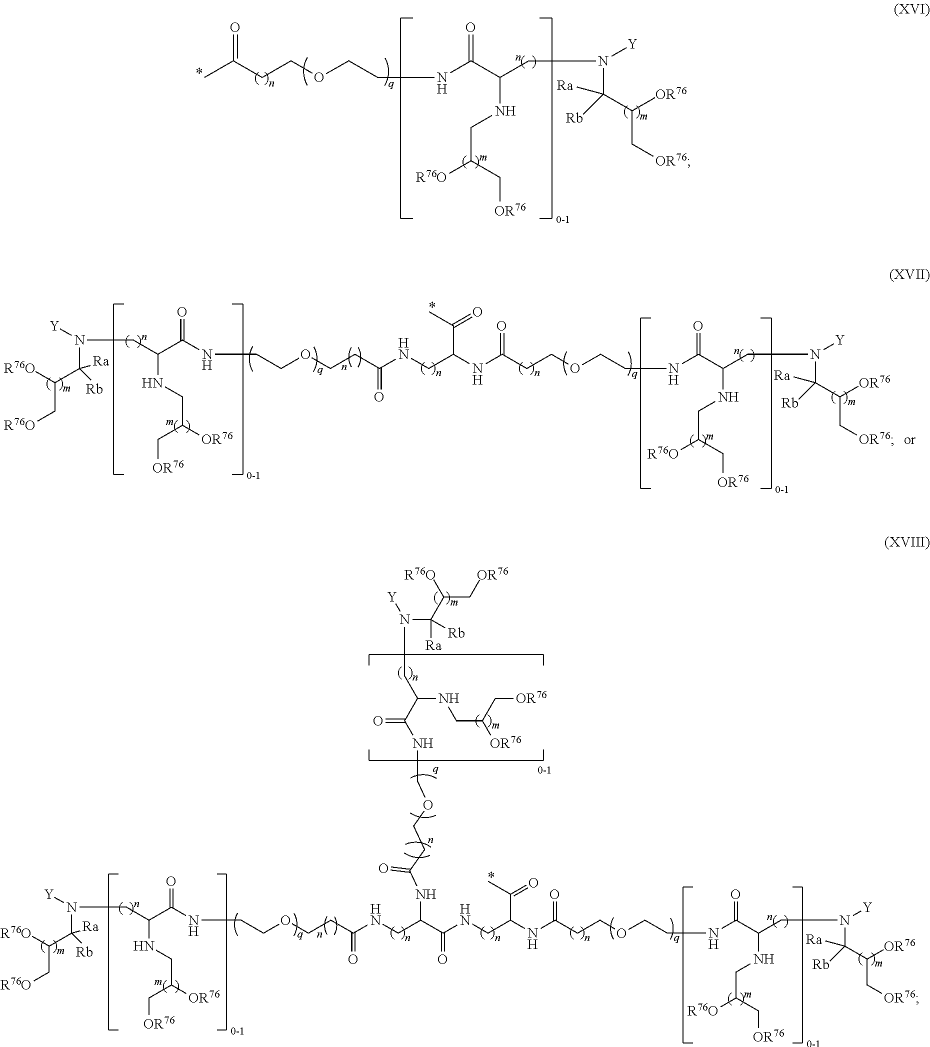

or a salt thereof, wherein:
   each Y is independently R⁷⁶ or;

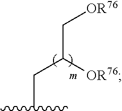

each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 1-26;
each m is independently 1 to 4;
each n is independently 1 to 4;
each v is independently 1 to 6; and
each * indicates an attachment site for a subunit of the Amino Acid unit (AA), the Linker subunit L2, or the Stretcher unit (L1).

In some embodiments, provided is a Linker intermediate or Linker, comprising a PEG unit having a formula selected from:

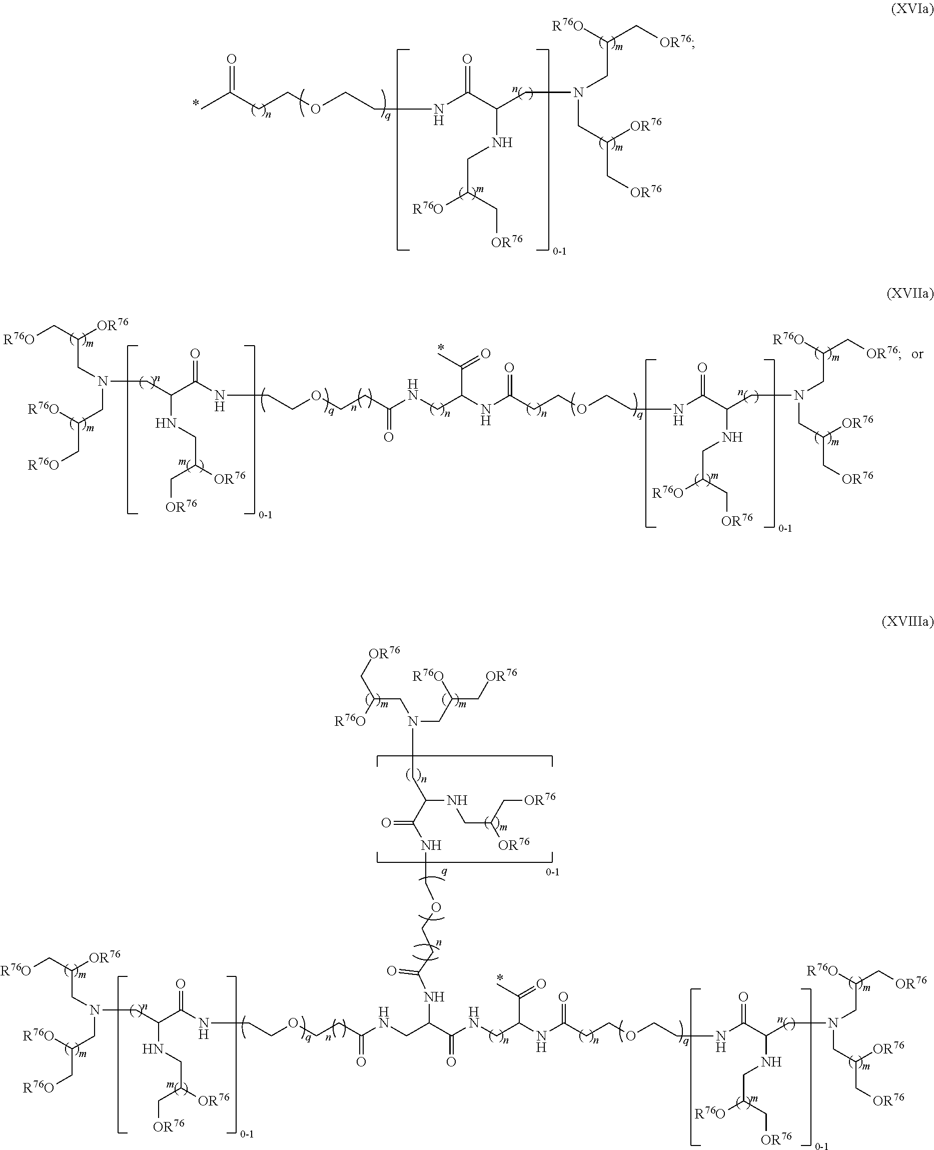

or a salt thereof, wherein:
   each R⁷⁶ is independently H, acetyl, —P(═O)(OH)₂, or —(CH₂)ᵥS(═O)₂(OH);
   each q is independently 1-26;
   each m is independently 1 to 4;
   each n is independently 1 to 4;
   each v is independently 1 to 6; and
   each * indicates an attachment site for a subunit of the Amino Acid unit (AA),
   the Linker subunit L2, or the Stretcher unit (Li).

In some embodiments, provided is a Linker intermediate or Linker, comprising a PEG unit having a formula selected from:

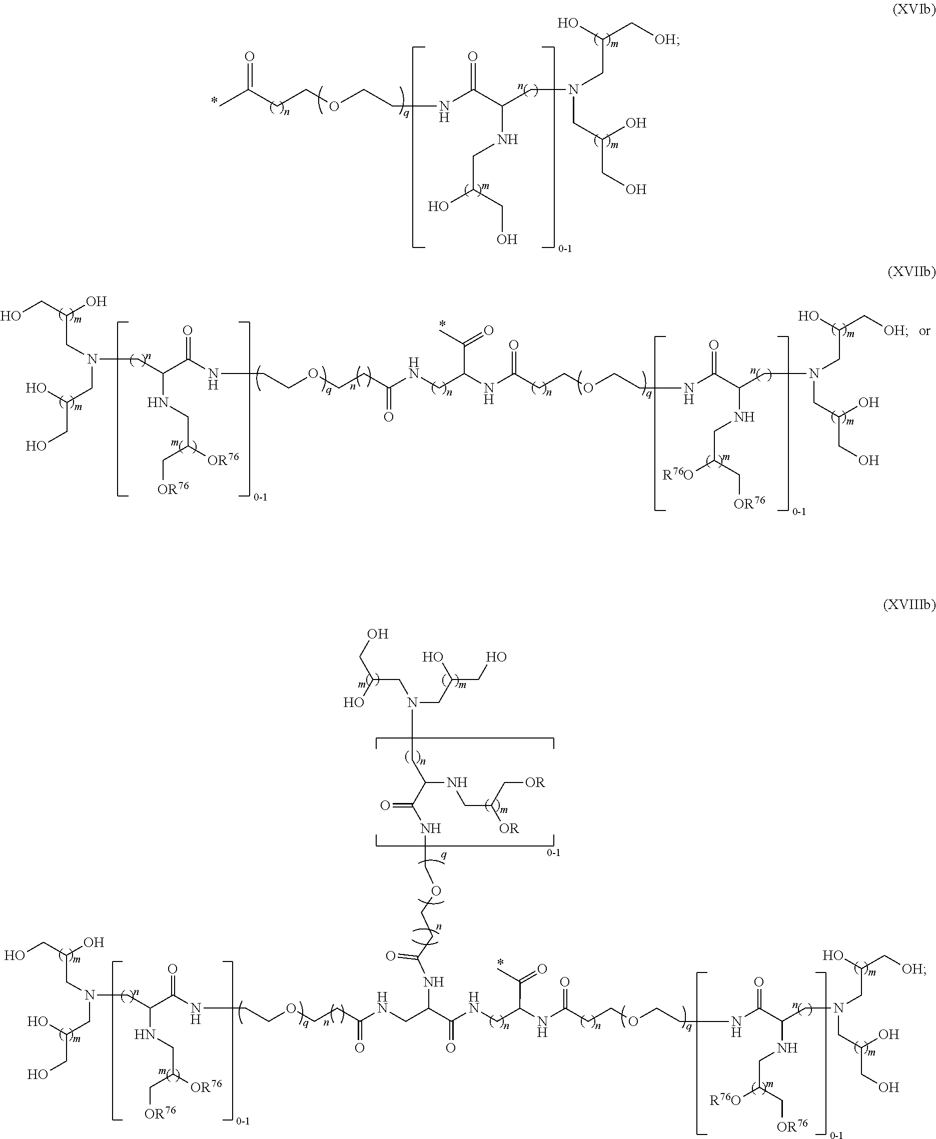

or a salt thereof, wherein:
   each q is independently 1-26;
   each m is independently 1 to 4;
   each n is independently 1 to 4; and
   each * indicates an attachment site for a subunit of the Amino Acid unit (AA), the Linker subunit L2, or the Stretcher unit (L1).

In some embodiments, provided is a Linker intermediate or Linker, wherein Y is R⁷⁶.

In some embodiments, provided is a Linker intermediate or Linker, wherein Y is

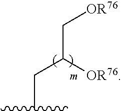

In some embodiments, provided is a Linker intermediate or Linker, wherein each Rₐ and R_{b} is independently H.

In some embodiments, provided is a Linker intermediate or Linker, wherein Rₐ and R_{b}are taken together with the carbon to which they are attached to form an oxo group.

In some embodiments, provided is a Linker intermediate or Linker, wherein q is 10-20.

In some embodiments, provided is a Linker intermediate or Linker, wherein q is 12.

In some embodiments, provided is a Linker intermediate or Linker, wherein the PEG unit is selected from the following, or a salt thereof:

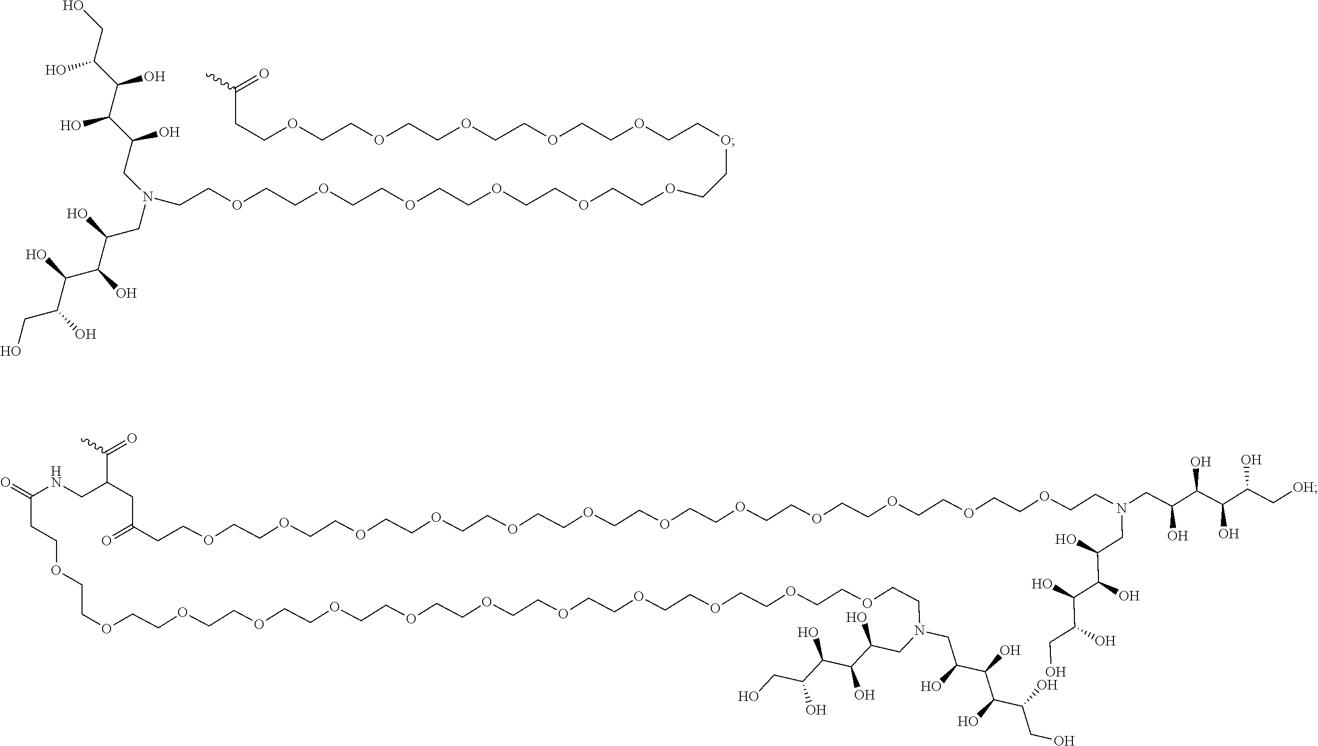

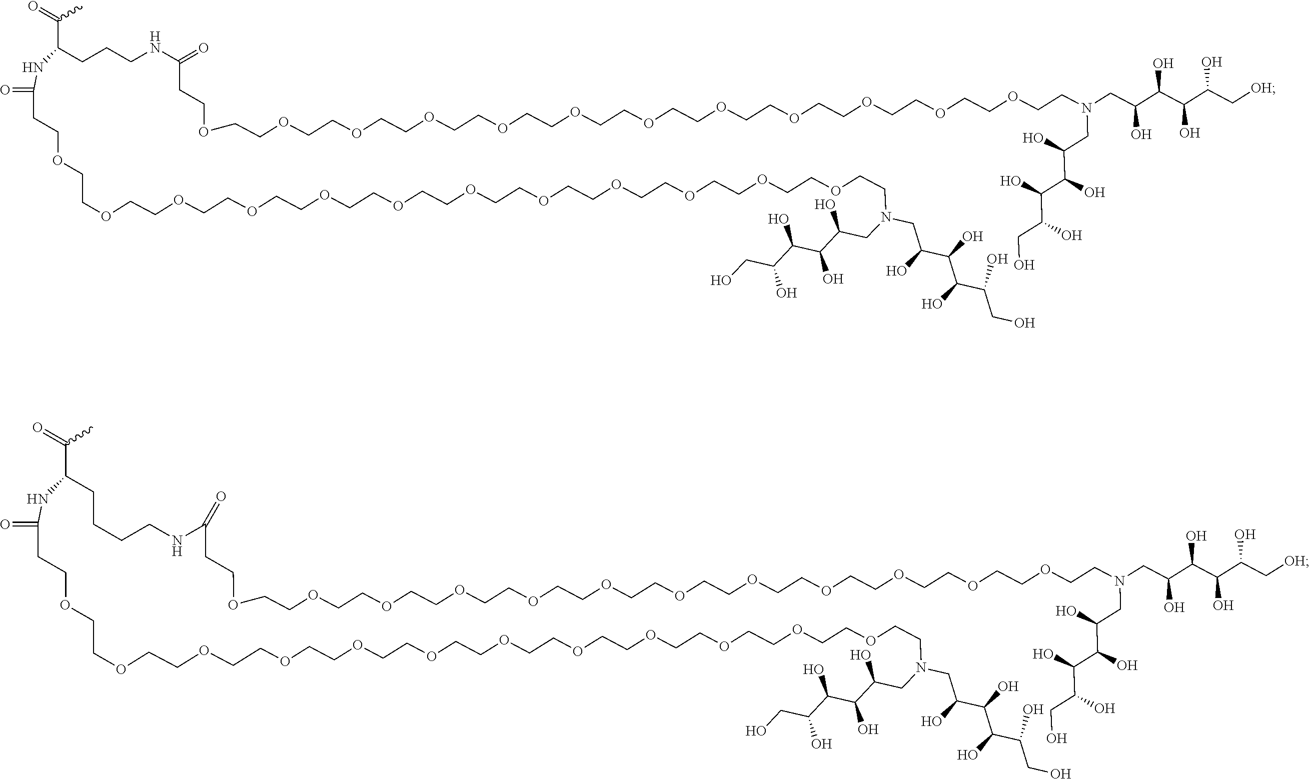

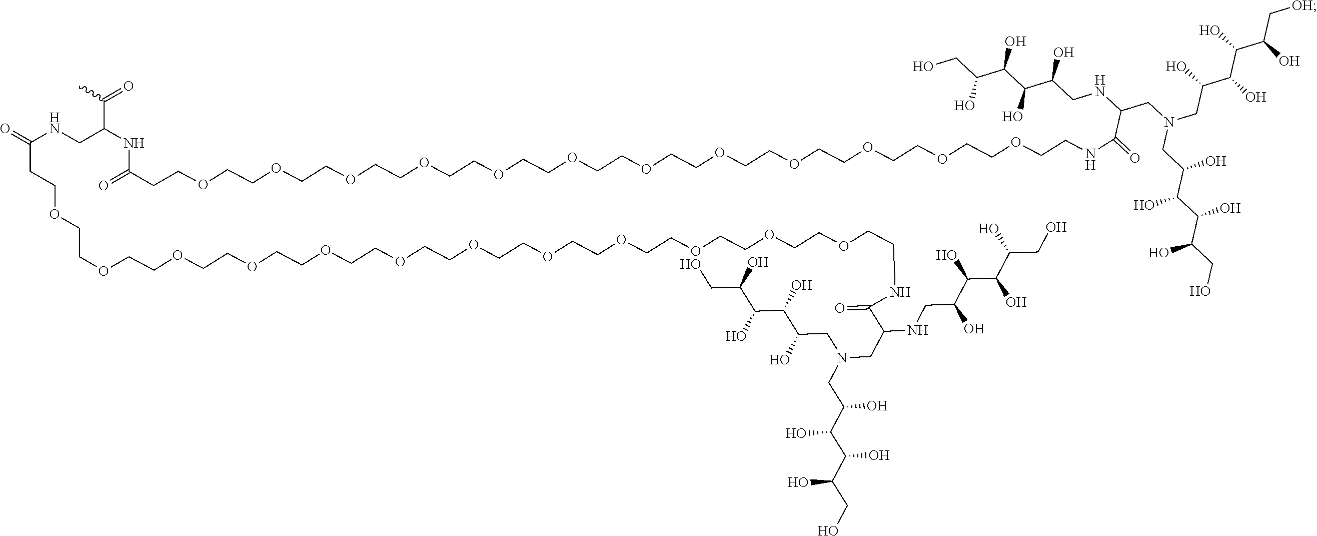

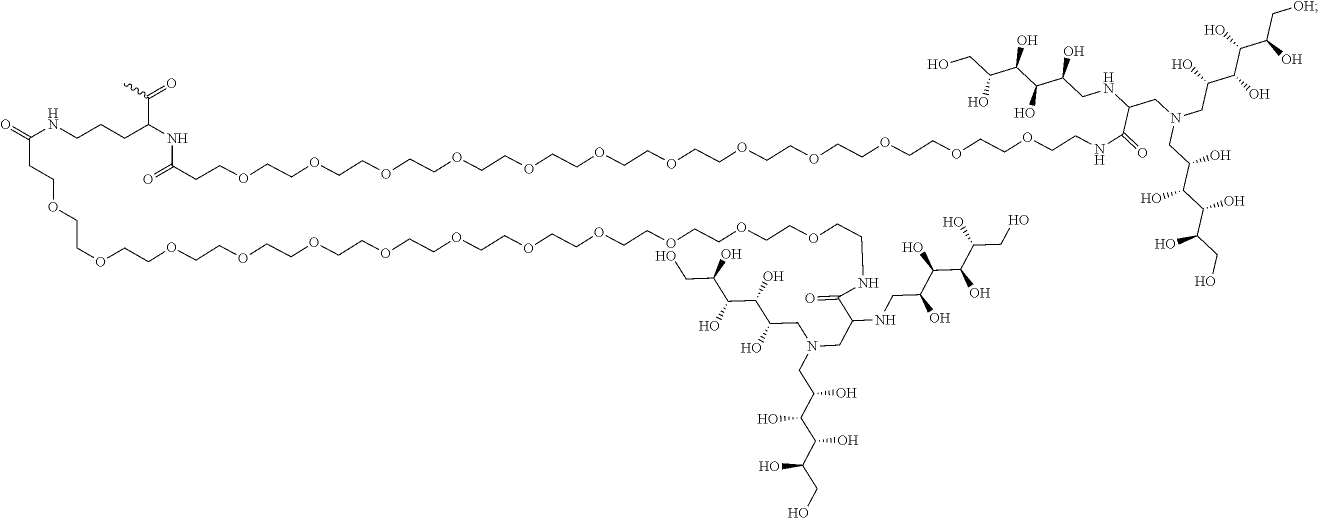

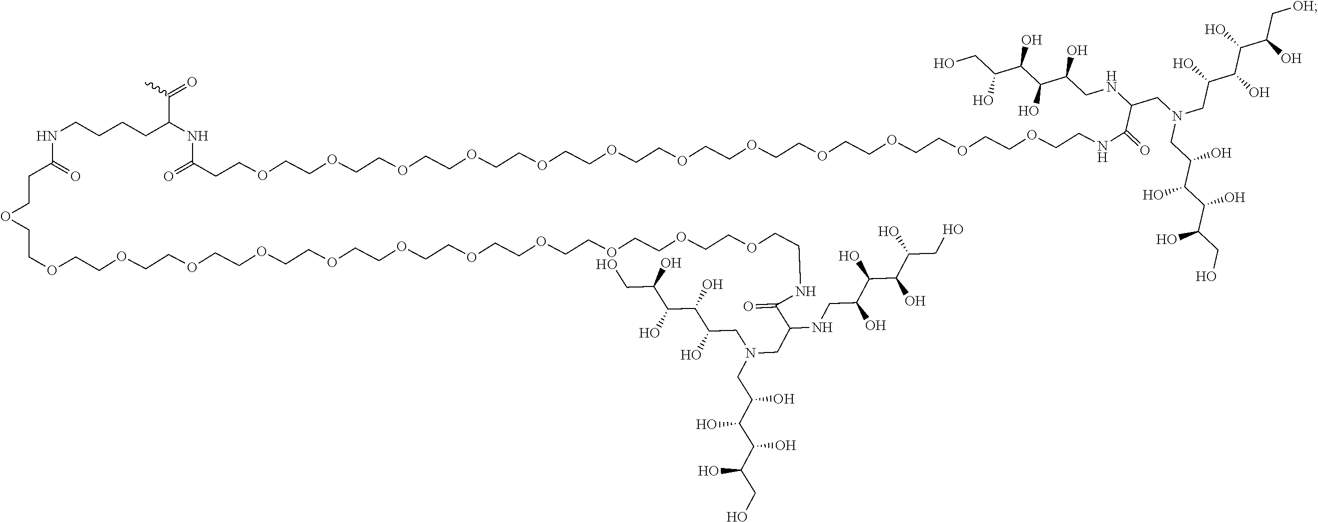

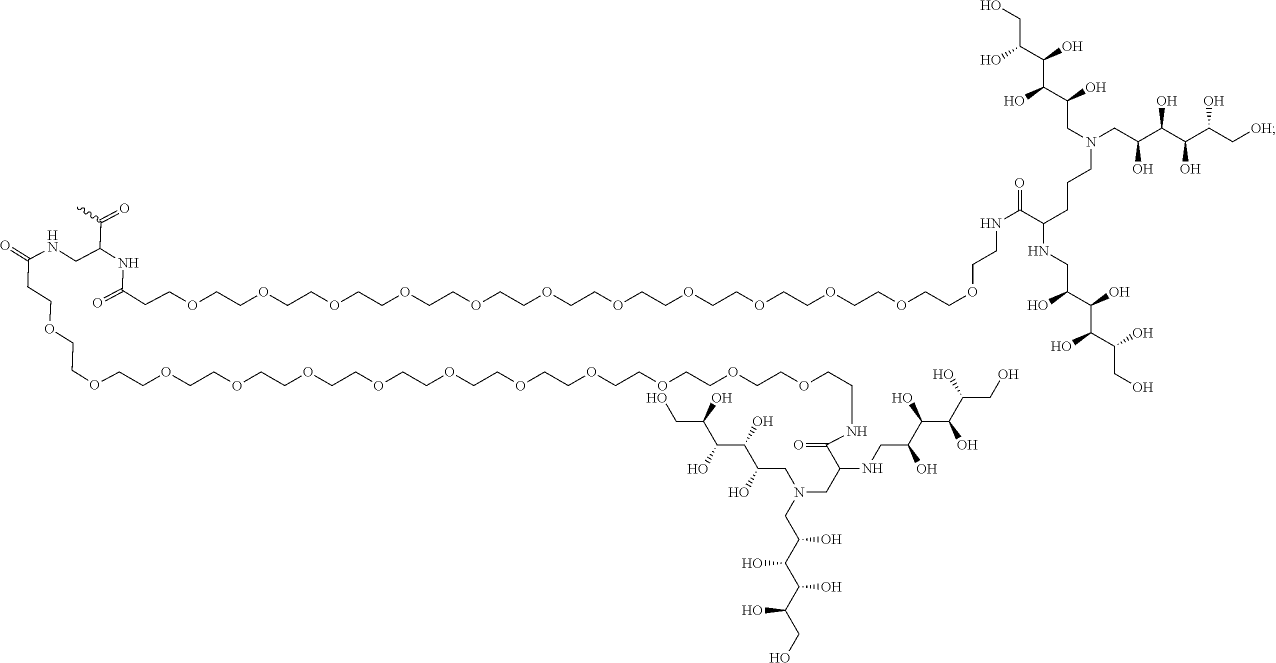

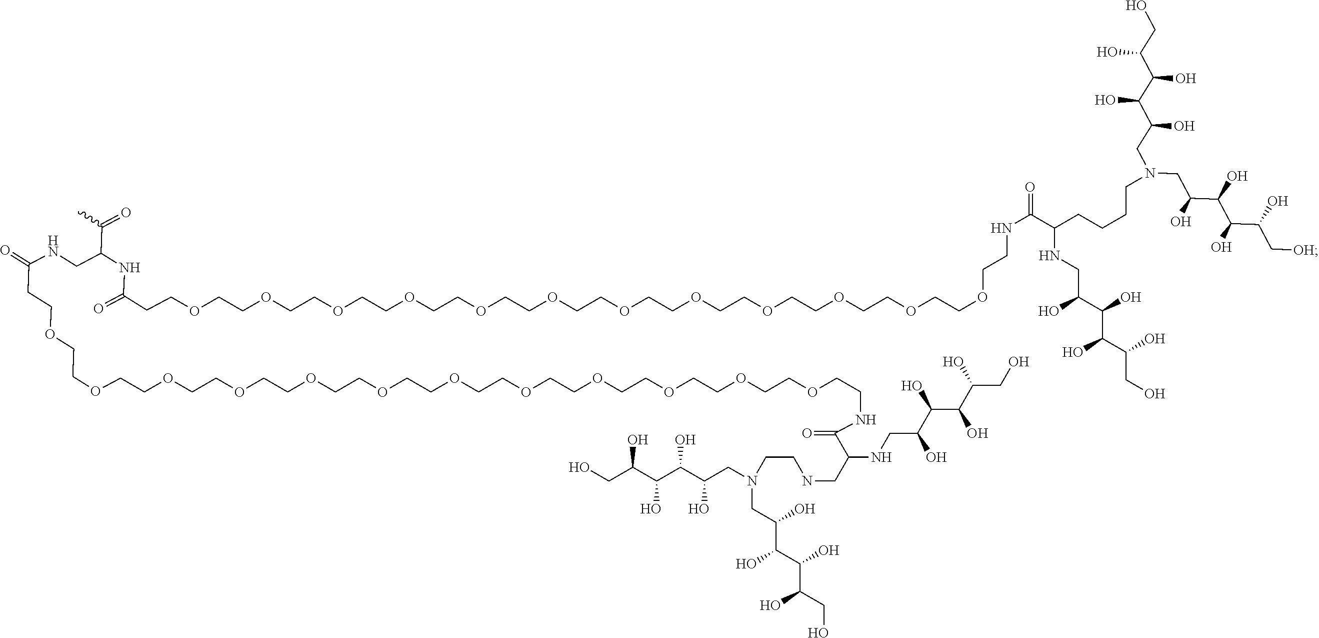

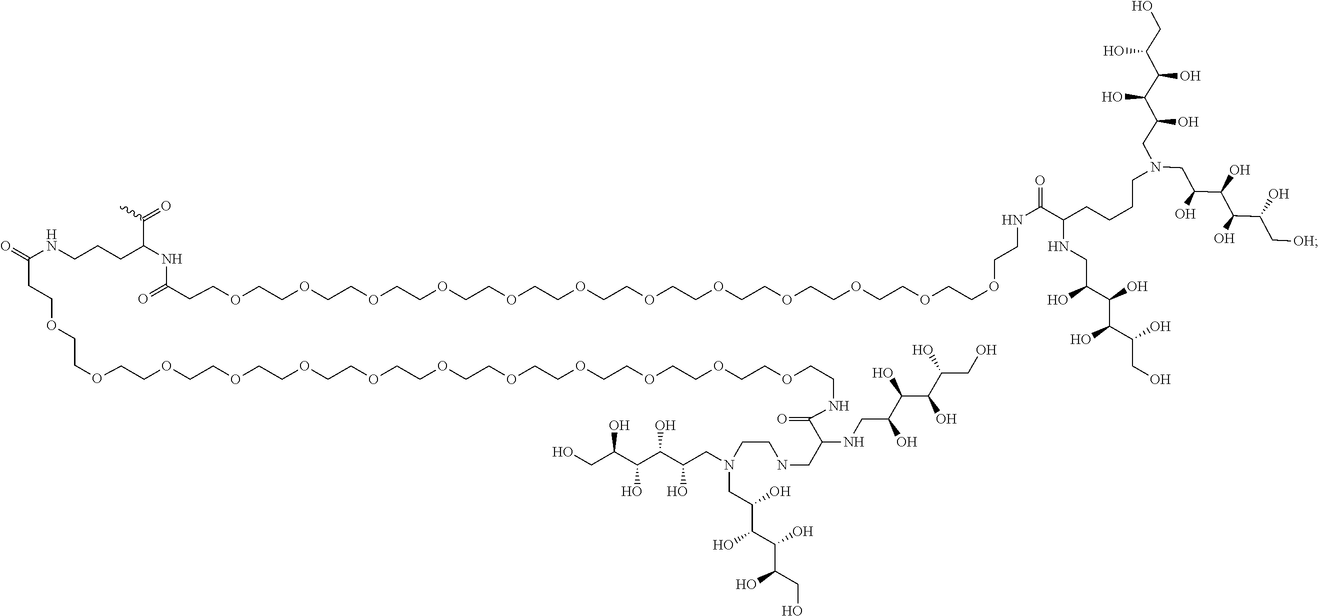

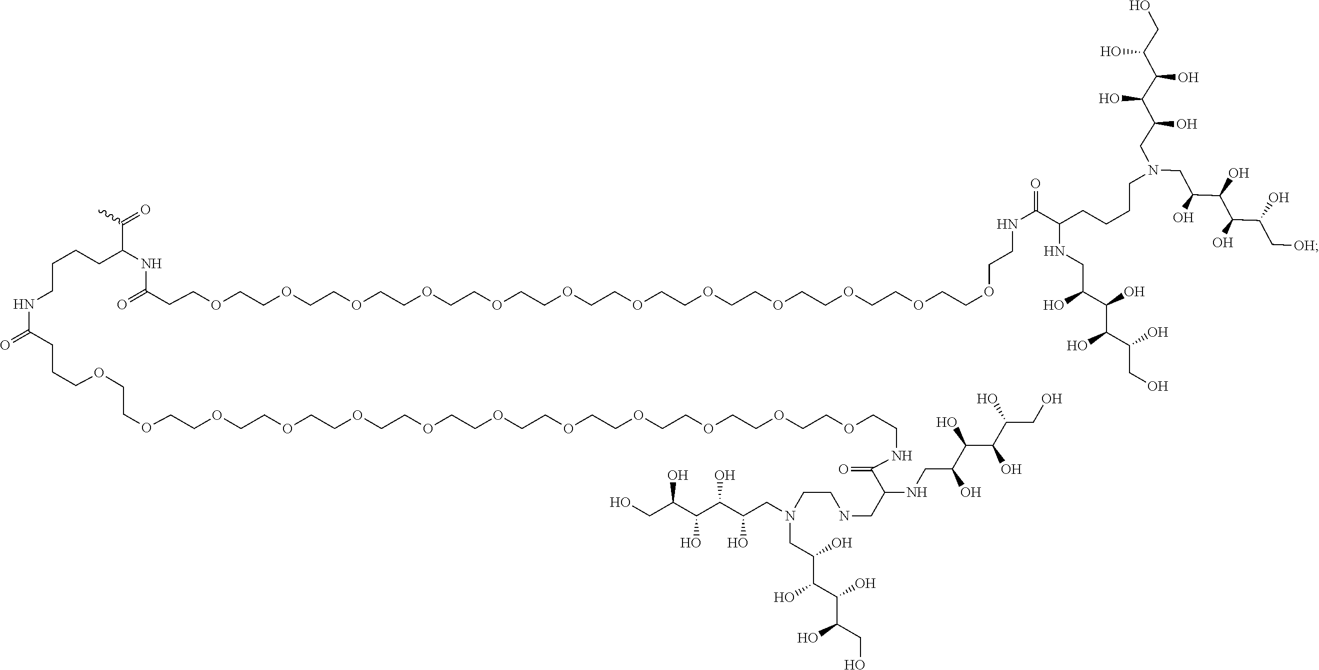

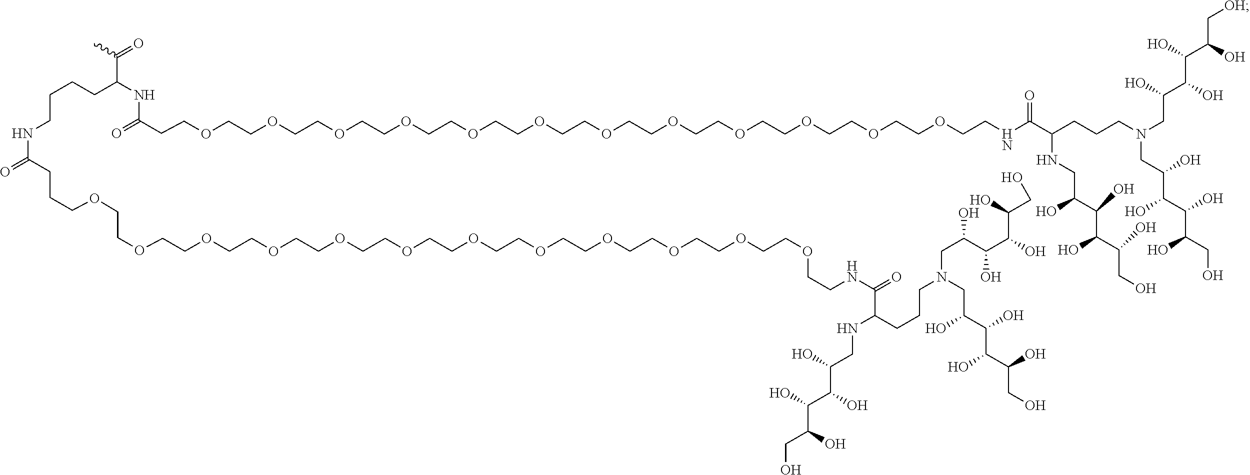

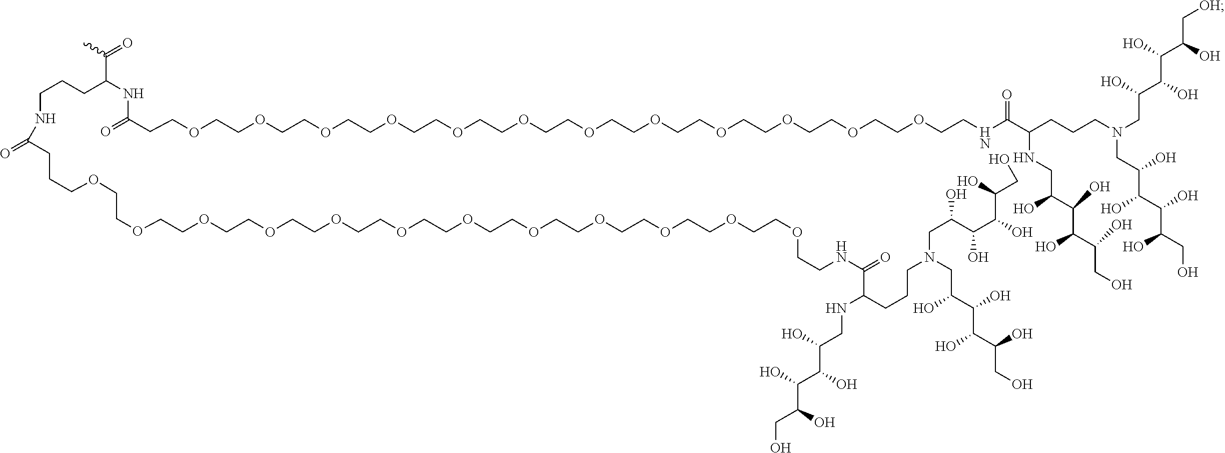

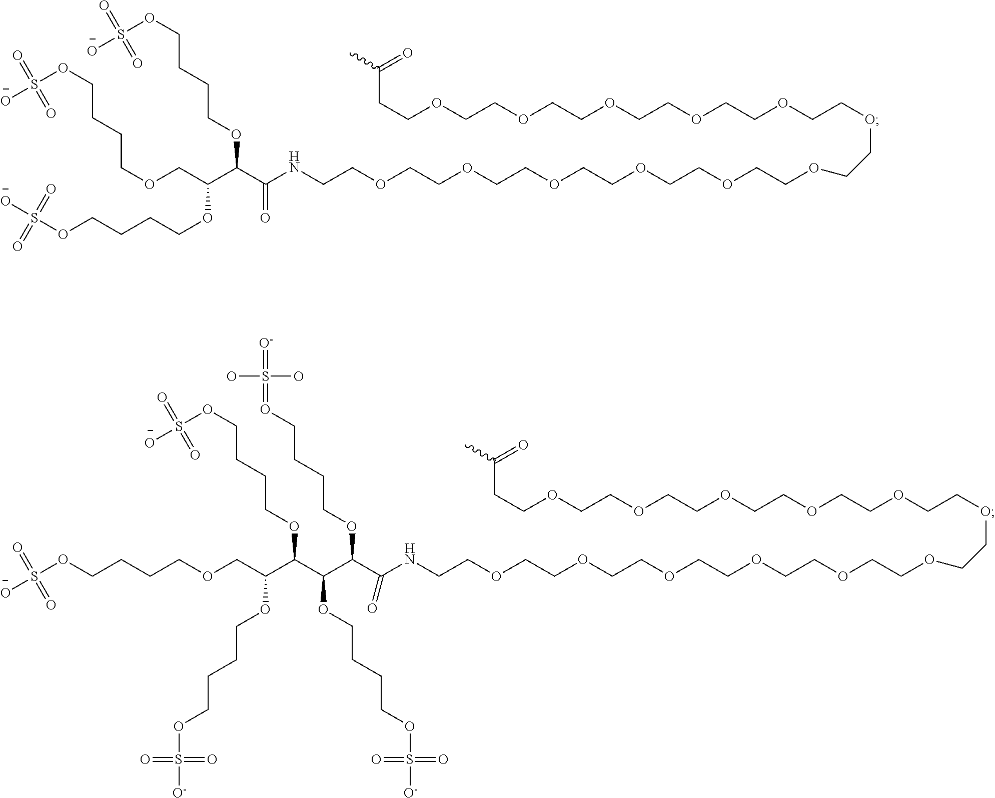

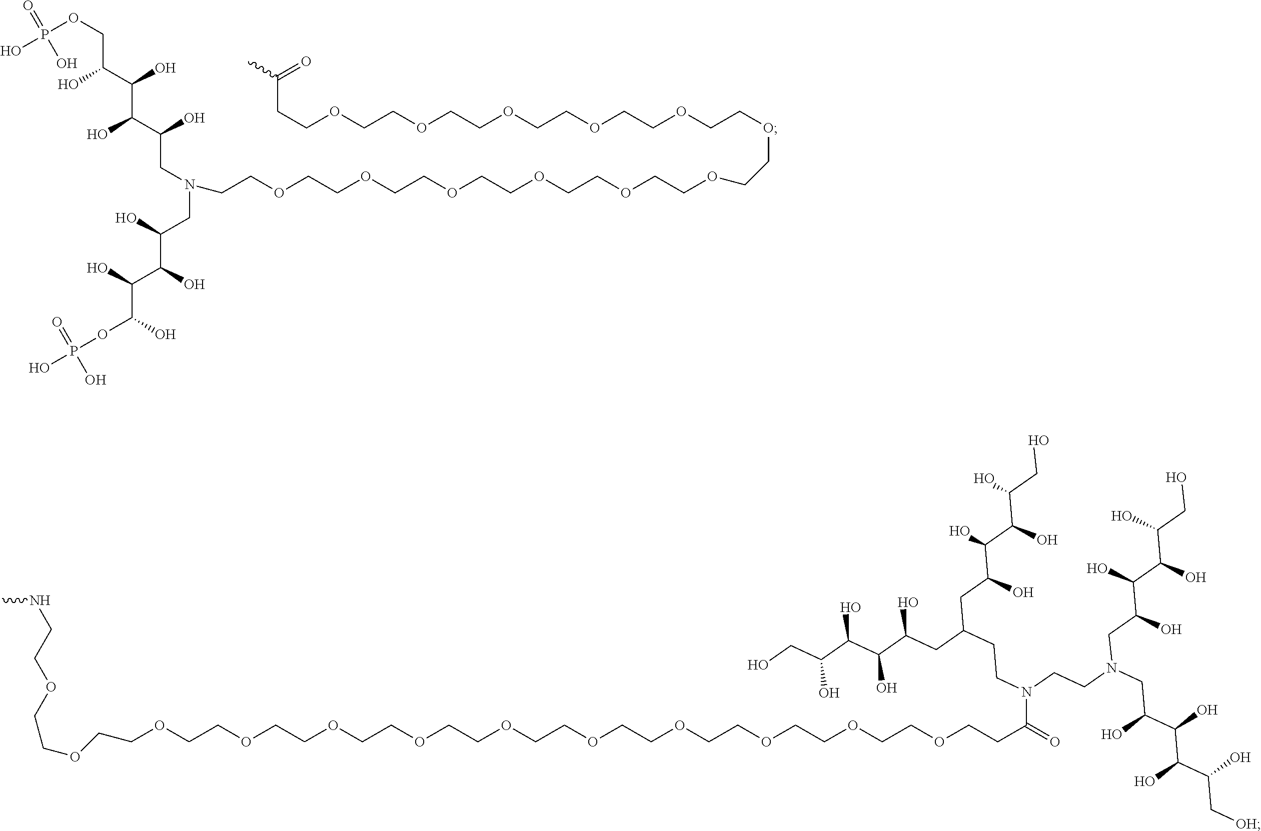

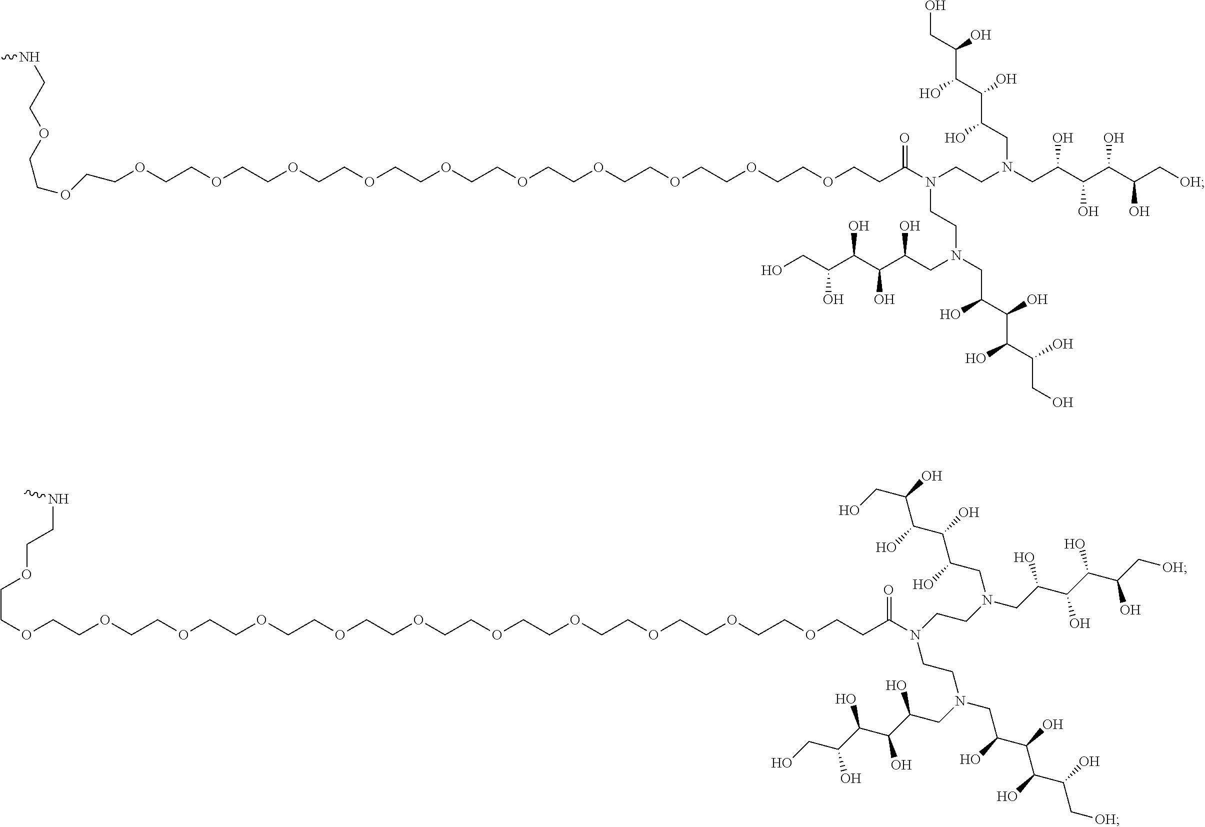

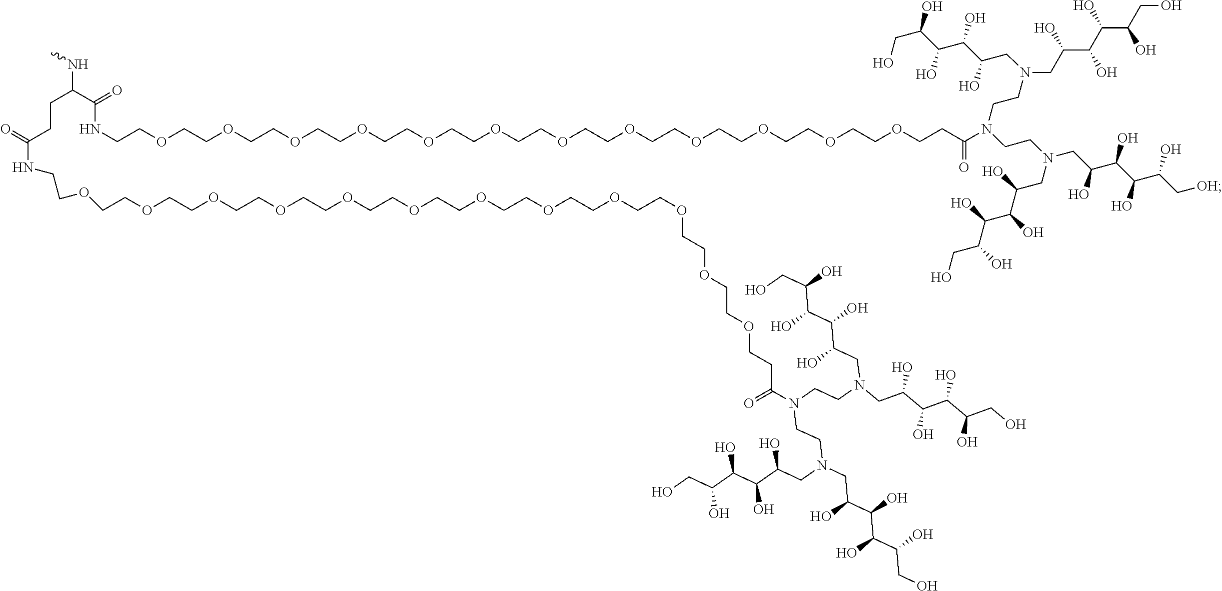

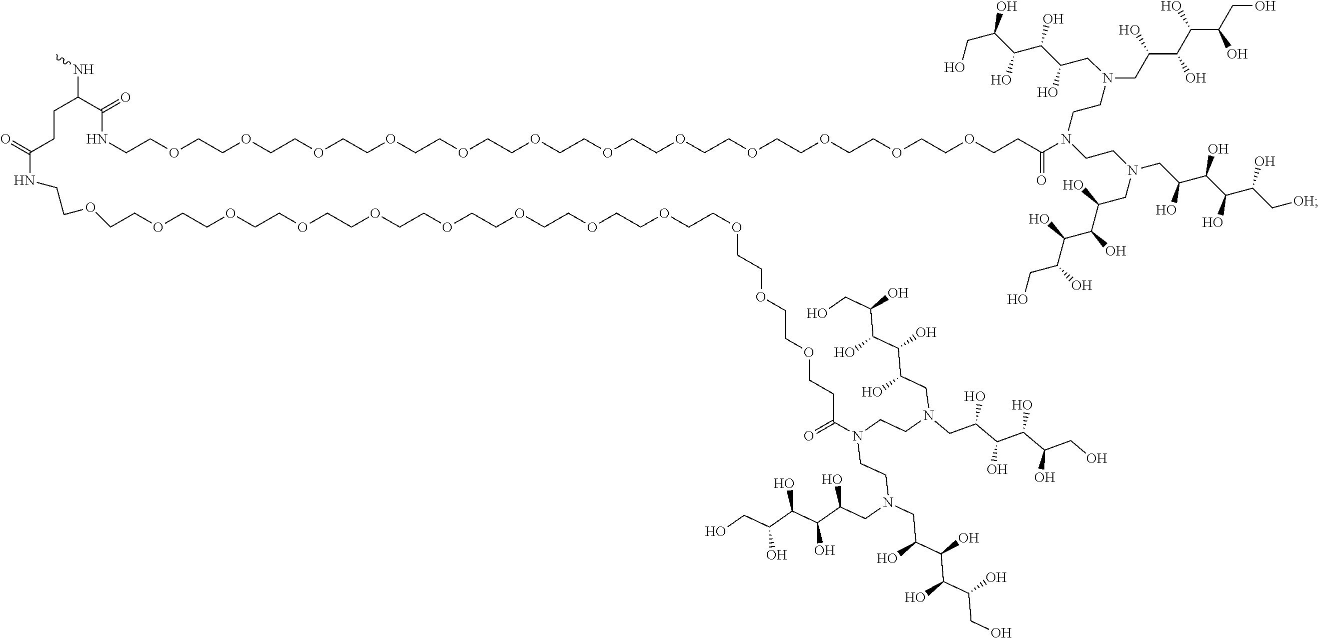

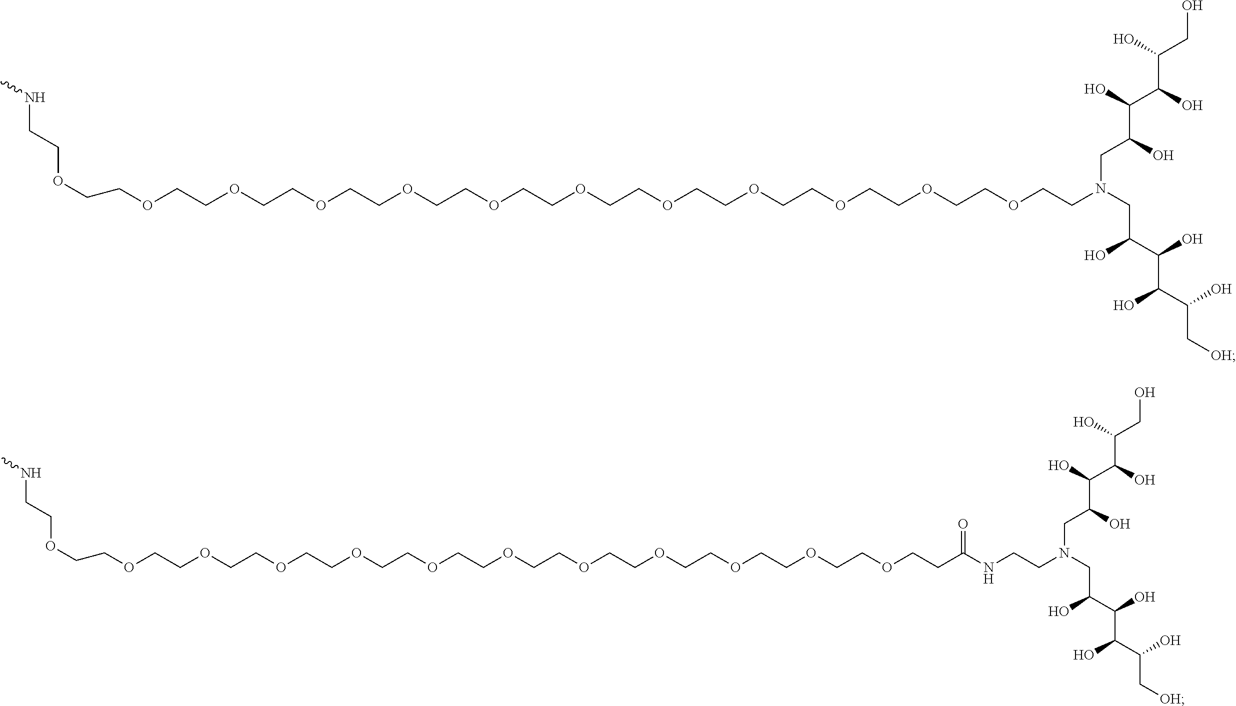

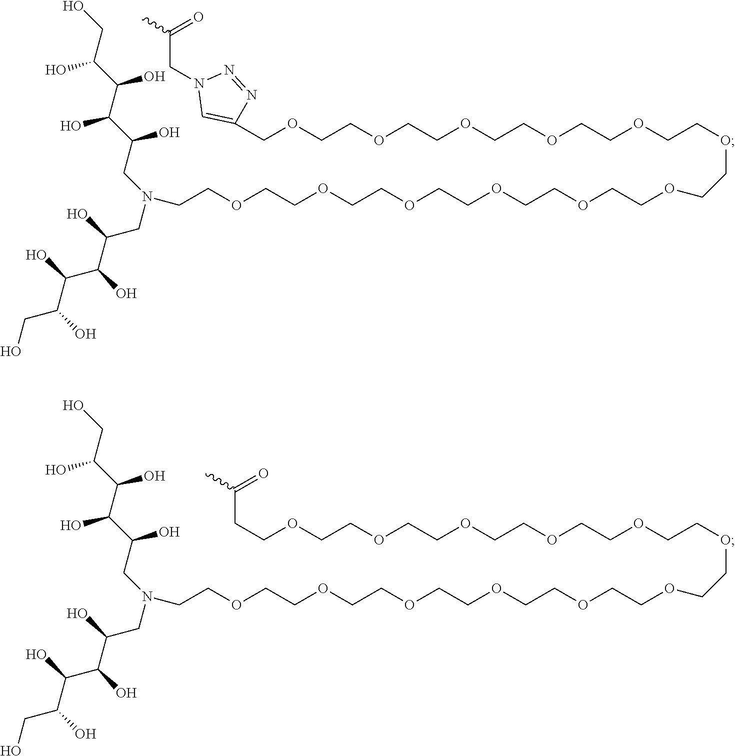

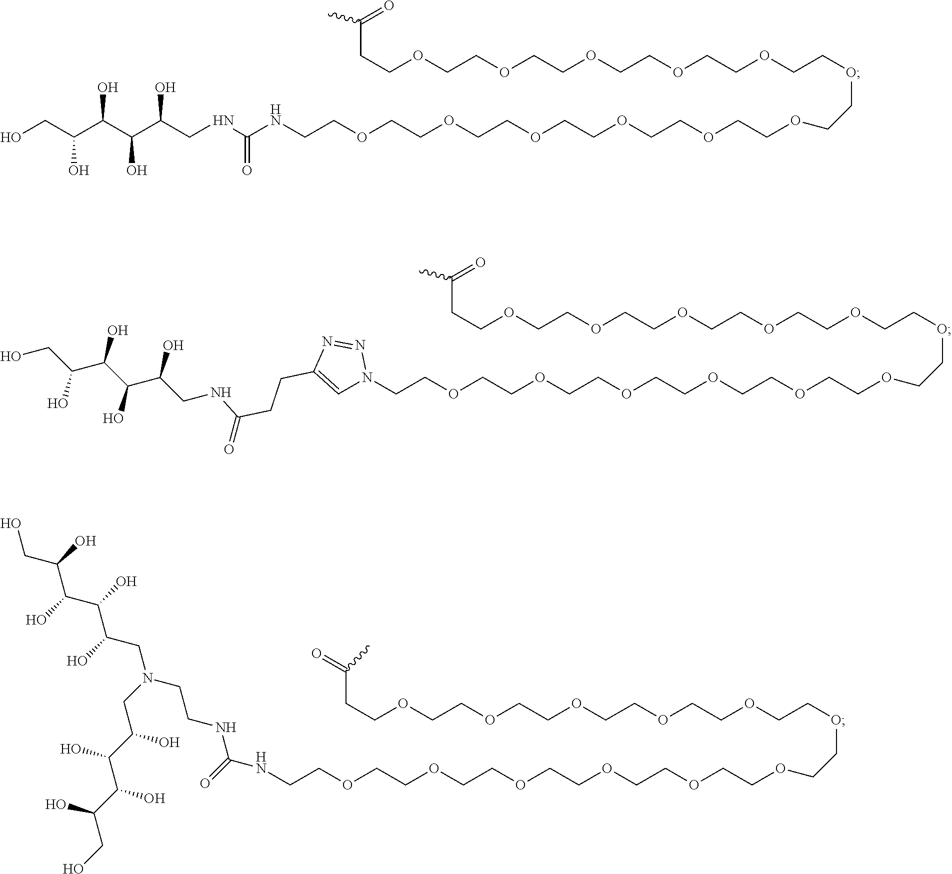

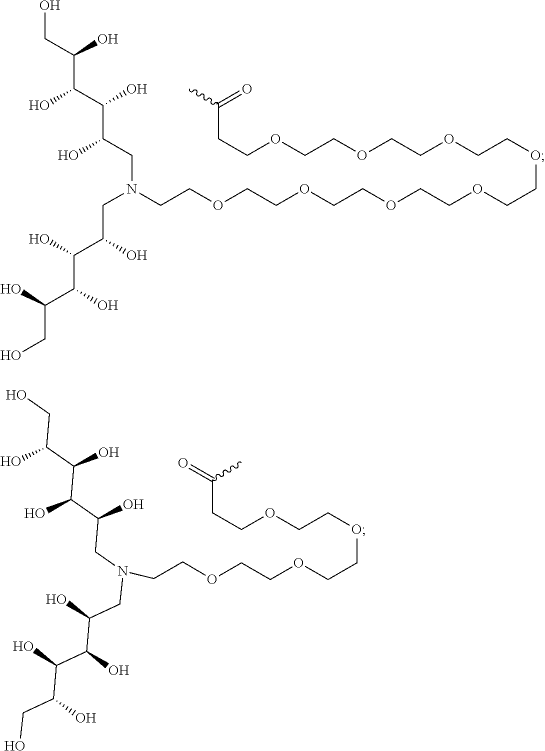

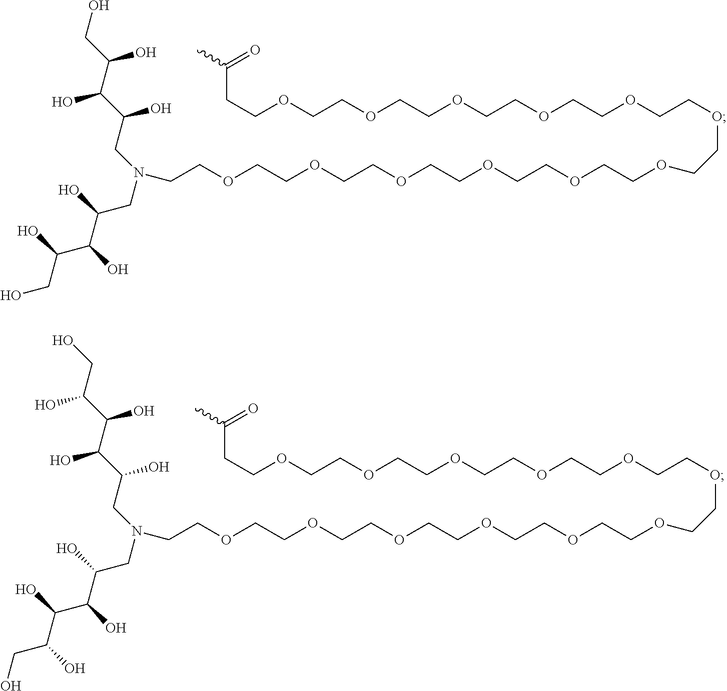

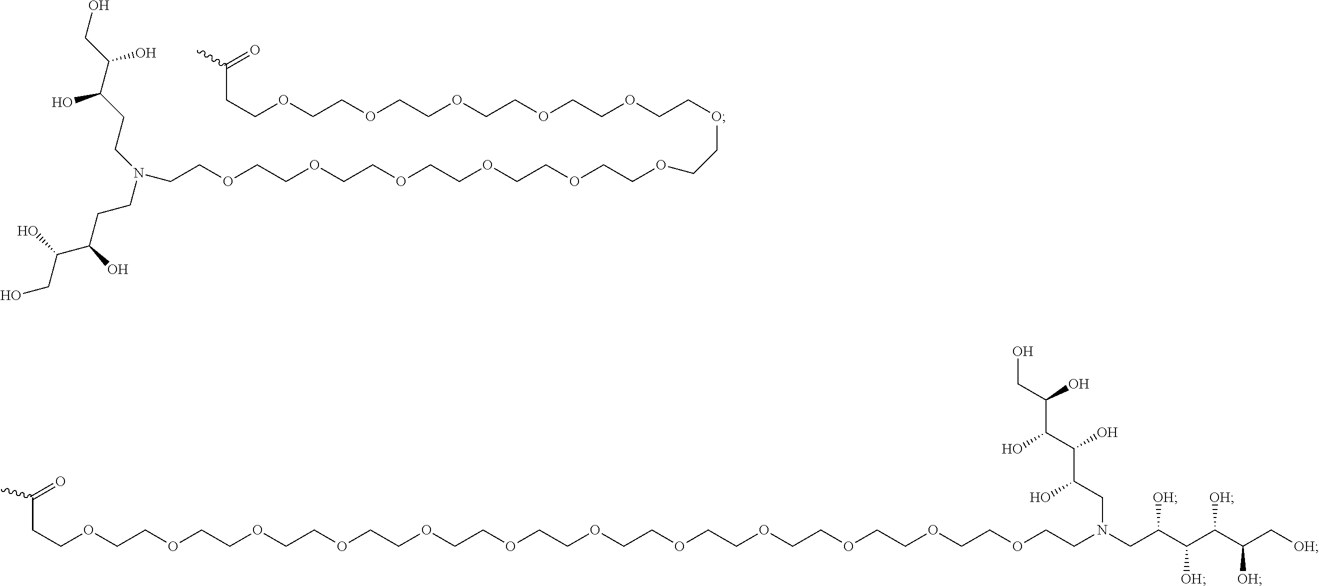

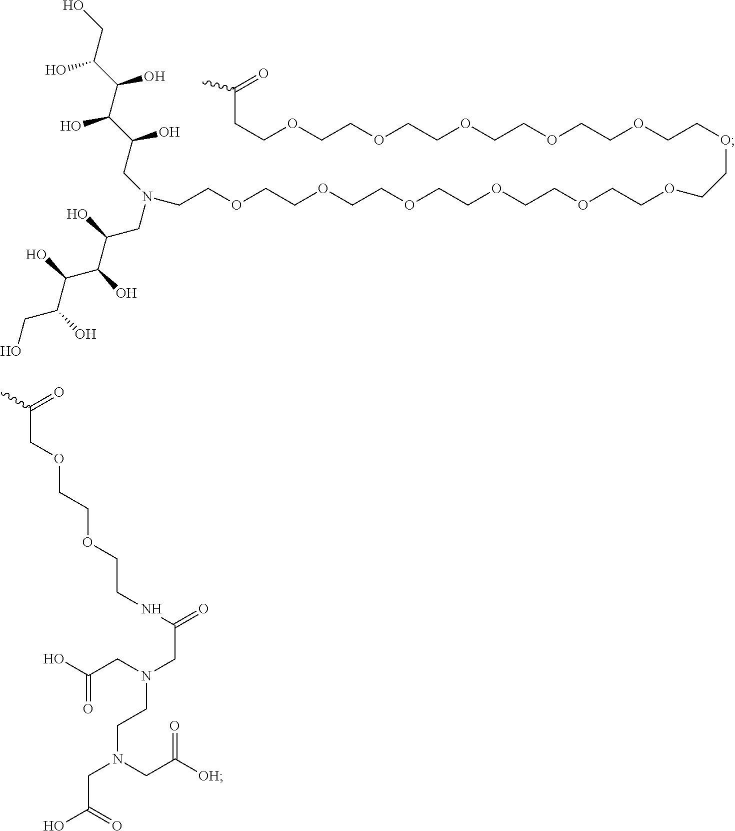

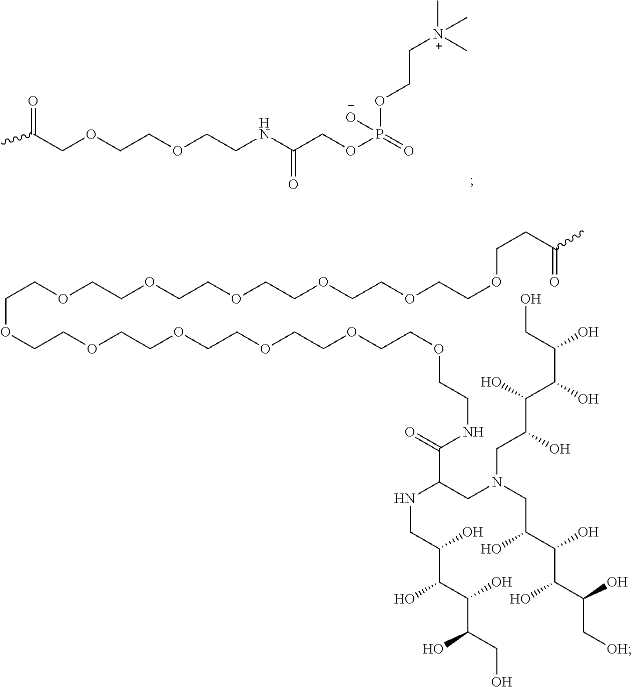

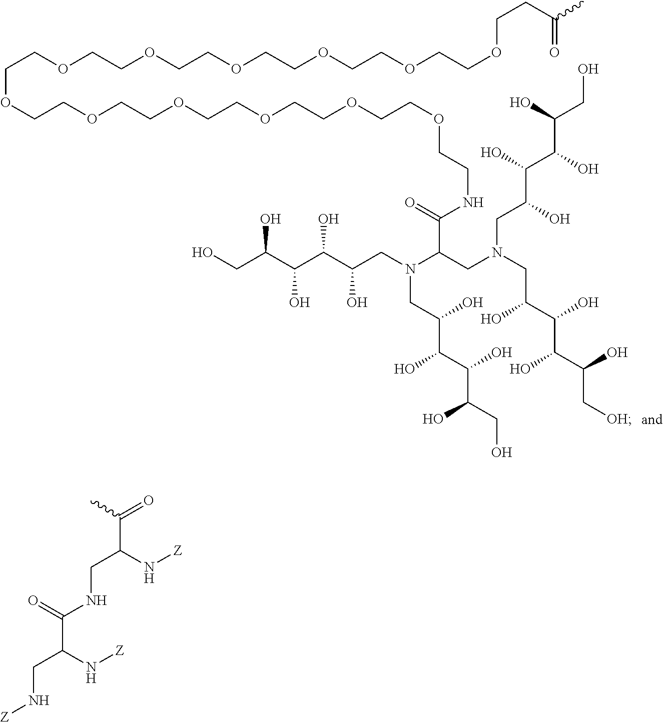

wherein each Z is attached at * and is individually selected from:

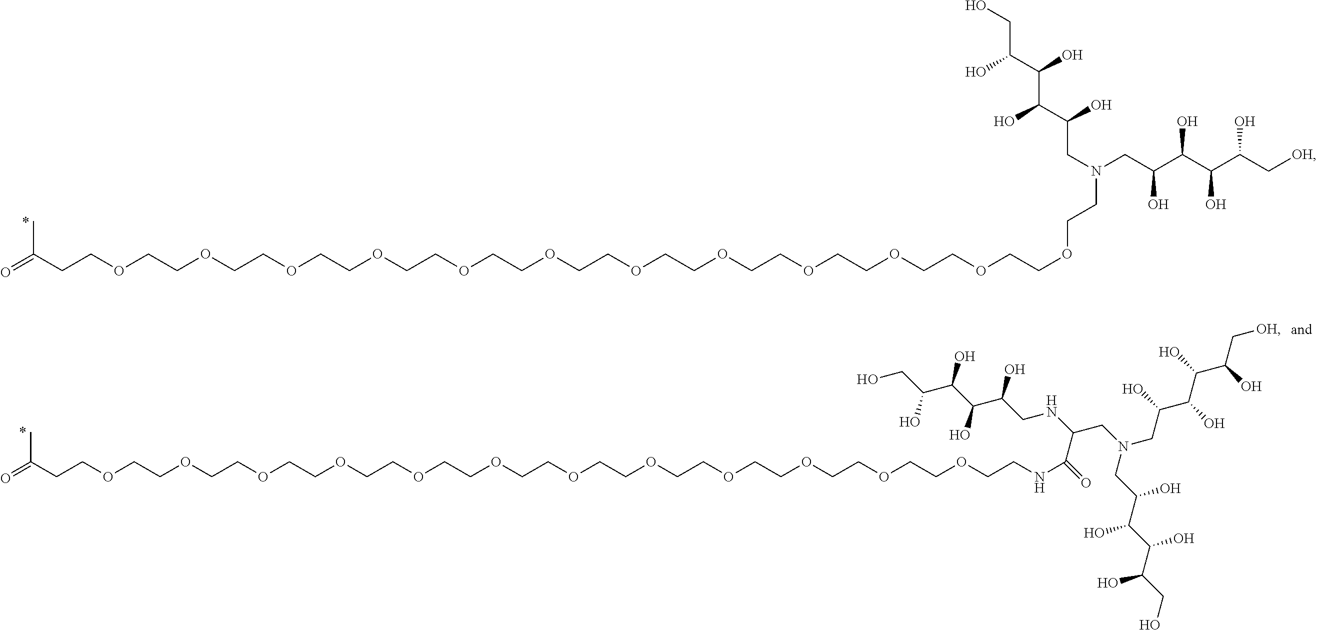

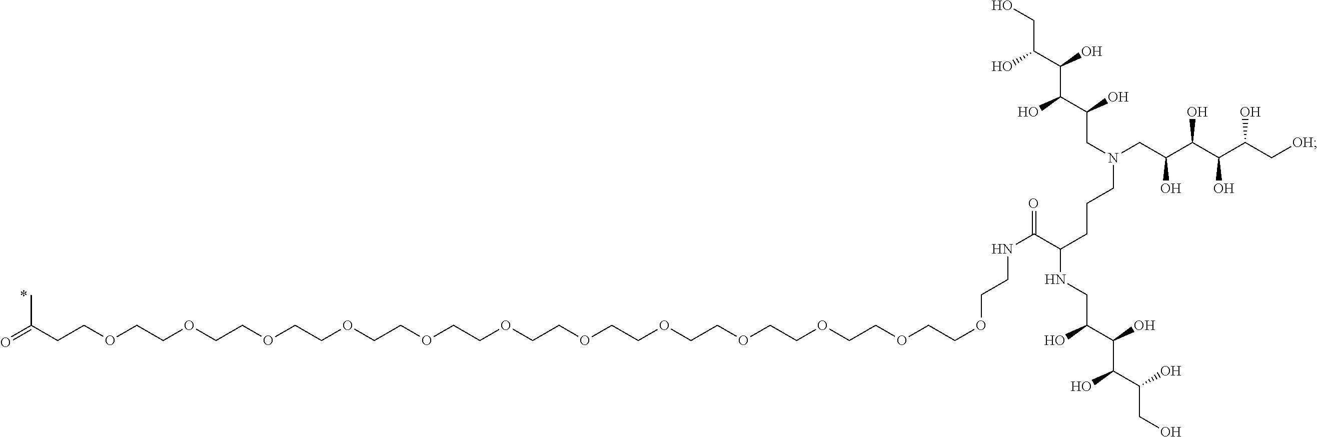

wherein each indicates an attachment site for another subunit of the Amino Acid unit [symbol] (AA), the Linker subunit L2, or the Stretcher unit (L1).

### Carboxyl Unit

In some embodiments, provided is a Linker intermediate or Linker, wherein the Carboxyl unit has the following formula:

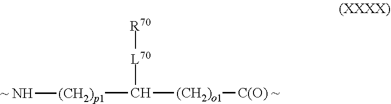

or a salt thereof, wherein:
(a)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
R⁷⁰ is —NR⁷¹(R⁷²-R⁷³), wherein R⁷¹ is selected from H, C₁-C₁2 alkyl, substituted C₁-C₁₂ alkyl, or polyethylene glycol (optionally having 1 to 12 ethylene glycol subunits), R⁷² is absent or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
   each wavy line (˜) indicates an attachment site for another subunit of an Amino Acid unit (AA), the Linker subunit L2, or the Stretcher unit (L1); and each of p1 and 01 are independently selected from 0 to 2;
   or
(b)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is —NR⁷¹(R⁷⁵_(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; each wavy line (˜) indicates an attachment site for another subunit of an Amino Acid unit (AA), the Linker subunit L2, or the Stretcher unit (L1); and each of p1 and 01 are independently selected from 0 to 2;
   or
(c)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is —N(R⁷⁴—R⁷³)(R⁷2.R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; each wavy line (˜) indicates an attachment site for another subunit of an Amino Acid unit (AA), the Linker subunit L2, or the Stretcher unit (L1); and each of p1 and 01 are independently selected from 0 to 2.

In some embodiments, provided is a Linker intermediate or Linker, comprising at least one Sugar unit. In some embodiments, provided is a Linker intermediate or Linker, comprising at least one PEG unit. In some embodiments, provided is a Linker intermediate or Linker, comprising at least one Carboxyl unit. In some embodiments, provided is a Linker intermediate or Linker, comprising at least two Polar units, each Polar unit selected from a Sugar unit, a PEG unit and a Carboxyl unit. In some embodiments, provided is a Linker intermediate or Linker, comprising at least one Sugar unit and a PEG unit or a Carboxyl unit. In some embodiments, provided is a Linker intermediate or Linker, comprising at least one Carboxyl unit and a PEG unit.

In some embodiments, provided is a Linker intermediate or Linker, wherein the Amino Acid unit (AA) is present (s=1). In some embodiments, provided is a Linker intermediate or Linker, wherein the Amino Acid unit comprises at least one Polar unit.

In some embodiments, provided is a Linker intermediate or Linker, wherein L2 or AA-L2 has one of the following structures:

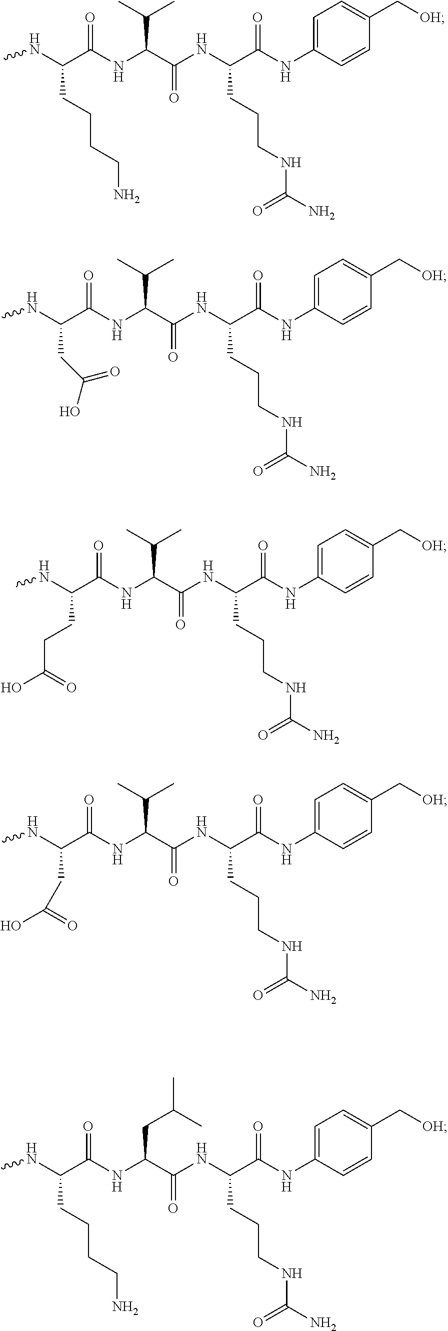

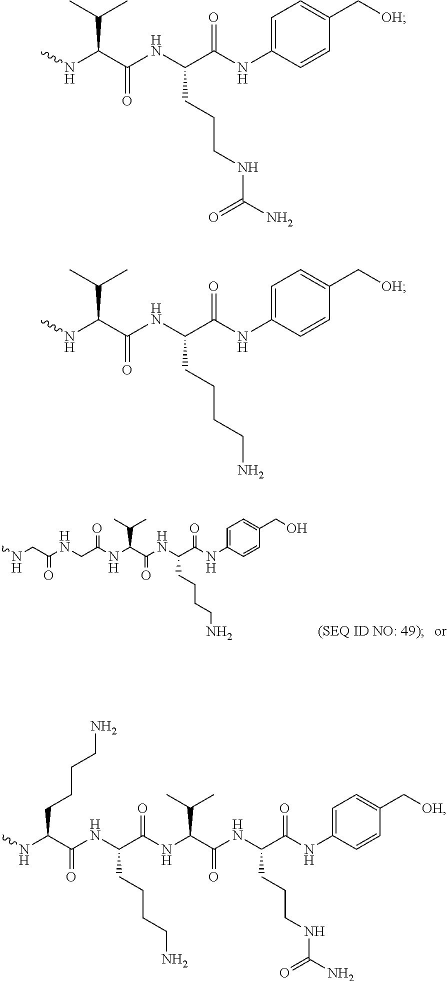

wherein the wavy line on the amino group indicates an attachment site for a Stretcher unit, and the Drug unit is attached to the benzyl alcohol.

In some embodiments, provided is a Linker intermediate or Linker, wherein ˜AA-L2˜ has a formula selected from the following:

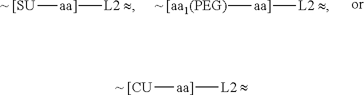

wherein the square brackets indicate the Amino Acid unit, each aa is an optional subunit of AA, L2 is the Linker Subunit, each wavy line (˜) indicates an attachment site for a Stretcher unit; aa₁(PEG) is a PEG unit attached to an amino acid subunit of AA, SU is a Sugar unit attached to a subunit of AA or to L2, and CU is a Carboxyl unit attached to a subunit of AA or to L2; and the double wavy (≈) line indicates an attachment site for a Drug unit, wherein aa and aa₁ are independently selected from alpha, beta and gamma amino acids and derivatives thereof.

In some embodiments, provided is a Linker intermediate or Linker, wherein ˜AA-L2˜ has a formula selected from the following:

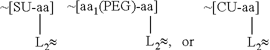

wherein the square brackets indicate the Amino Acid unit, each aa is an amino acid subunit of AA, L2 is the Linker Subunit attached to a side chain of aa, the wavy line (˜) indicates an attachment site for a Stretcher unit; aa₁(PEG) is a PEG unit attached to aa, SU is a Sugar unit attached to aa, CU is a Carboxyl unit attached to aa, and the double wavy (≈) line indicates an attachment site for a Drug unit; wherein aa and aa, are independently selected from alpha, beta and gamma amino acids and derivatives thereof.

In some embodiments, provided is a Linker intermediate or Linker, wherein the Amino Acid unit comprises at least two Polar units.

In some embodiments, provided is a Linker intermediate or Linker, wherein ˜AA-L2 has a formula selected from the following:

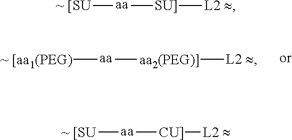

wherein the square brackets indicate the Amino Acid unit, aa is an optional subunit of AA, L2 is the Linker Subunit, the wavy line (˜) indicates an attachment site for a Stretcher unit; each of aa₁(PEG) and aa₂(PEG) is a PEG unit attached to aa or to the other PEG unit; each SU is a Sugar unit attached to aa or the other Sugar unit, each CU is a Carboxyl unit attached to aa or to the other Carboxyl unit, and the double wavy (≈) line indicates an attachment site for a Drug unit; wherein aa, aa, and aa₂ are independently selected from selected from alpha, beta and gamma amino acids and derivatives thereof.

In some embodiments, provided is a Linker intermediate or Linker, wherein ˜AA-L2˜ has a formula selected from the following:

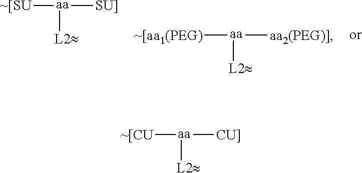

wherein the square brackets indicate the Amino Acid unit, aa is an amino acid subunit of AA, L2 is a Linker Subunit attached to a side chain of aa, each wavy line (˜) indicates an attachment site for a Stretcher unit; each of aa₁(PEG) and aa₂(PEG) is a PEG unit attached to aa, each SU is a Sugar unit attached to aa; each CU is a Carboxyl unit attached to aa; and the double wavy (≈) line indicates an attachment site for a Drug unit; wherein each of aa, aa₁ and aa₂ is independently selected from alpha, beta and gamma amino acids and derivatives thereof.

In some embodiments, provided is a Linker intermediate or Linker, wherein Linker Subunit L2 is a cleavable linker unit. In some embodiments, provided is a Linker intermediate or Linker, wherein Linker Subunit L2 comprises a peptide that is cleavable by an intracellular protease. In some embodiments, provided is a Linker intermediate or Linker, wherein the cleavable peptide comprises a valine-citrulline peptide, a valine-alanine peptide, a valine-lysine peptide, a phenylalanine-lysine peptide, or a glycine-glycine-phenylalanine-glycine peptide (SEQ ID NO: 43).

In some embodiments, provided is a Linker intermediate or Linker, wherein Linker Subunit L2 comprises at least one Polar unit. In some embodiments, provided is a Linker intermediate or Linker, wherein the Polar unit is a Sugar unit (SU). In some embodiments, provided is a Linker intermediate or Linker, wherein the cleavable peptide comprises a SU-valine-citrulline peptide, a SU-valine-lysine peptide, a SU-valine-alanine peptide, a SU-phenylalanine-lysine peptide, or a SU-glycine-glycine-phenylalanine-glycine peptide (SEQ ID NO: 44).

In some embodiments, provided is a Linker intermediate or Linker, wherein the Polar unit is a Carboxyl unit (CU). In some embodiments, provided is a Linker intermediate or Linker, wherein the cleavable peptide comprises a CU-valine-citrulline peptide, a CU-valine-lysine peptide, a valine-(CU-lysine) peptide, a CU-valine-alanine peptide, a CU-phenylalanine-lysine peptide, a phenylalanine-(CU-lysine) peptide or a CU-glycine-glycine-phenylalanine-glycine peptide (SEQ ID NO: 45), wherein CU-lysine is a Carboxyl unit comprising a lysine residue.

In some embodiments, provided is a Linker intermediate or Linker, wherein the Polar unit is a PEG unit (PEG). In some embodiments, provided is a Linker intermediate or Linker, wherein the cleavable peptide comprises a Lys(PEG)-valine-citrulline peptide, a valine-Cit(PEG) peptide, a Lys(PEG)-valine-lysine peptide, a valine-lysine(PEG) peptide, a Lys(PEG)-valine-alanine peptide, a Lys(PEG)-phenylalanine-lysine peptide, a phenylalanine-Lys(PEG)) peptide or a Lys(PEG)-glycine-glycine-phenylalanine-glycine peptide (SEQ ID NO: 46), wherein Lys(PEG) and Cit(PEG) comprise a PEG unit attached to a lysine residue or a citrulline residue, respectively.

In some embodiments, provided is a Linker intermediate or Linker, wherein the cleavable peptide is attached to para-aminobenzyl alcohol self immolative group (PABA).

In some embodiments, provided is a Linker intermediate or Linker, wherein ˜AA-L2˜ has one of the following structures:

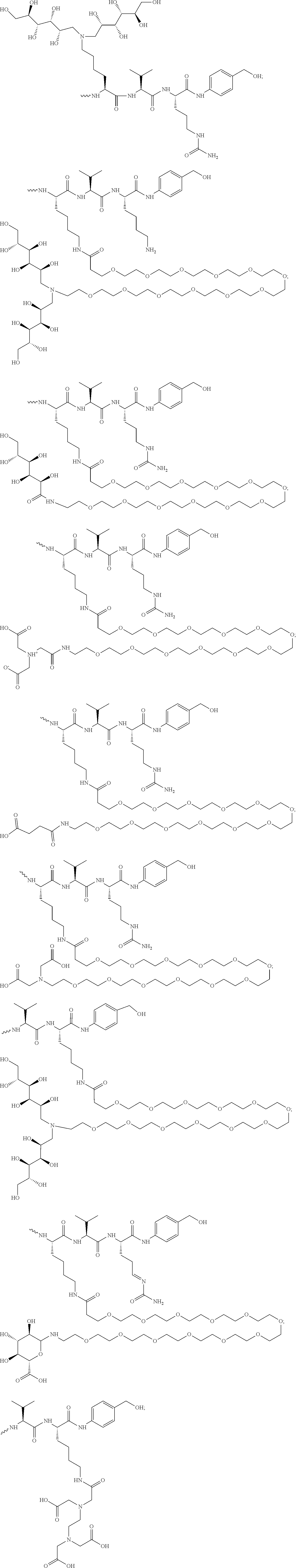

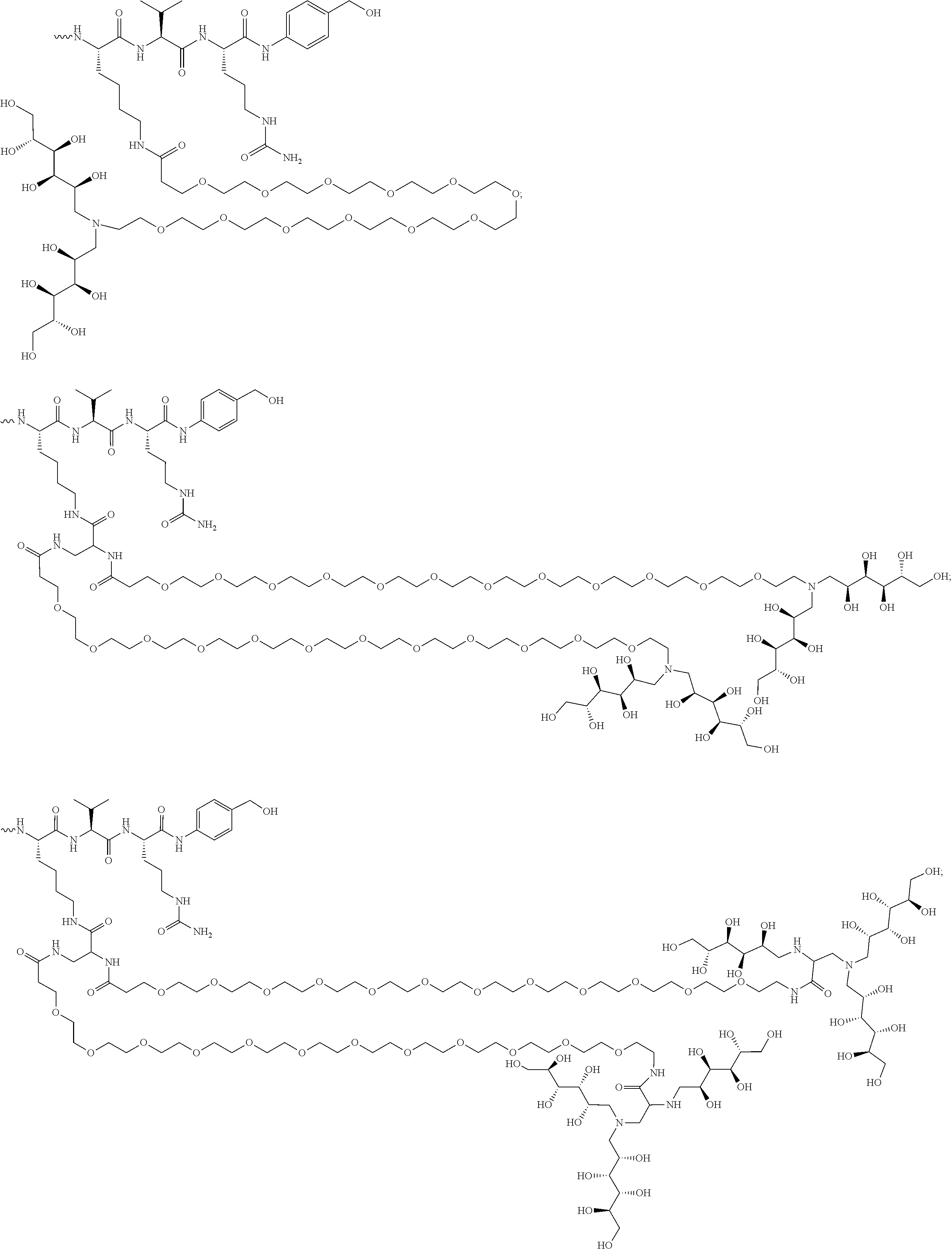

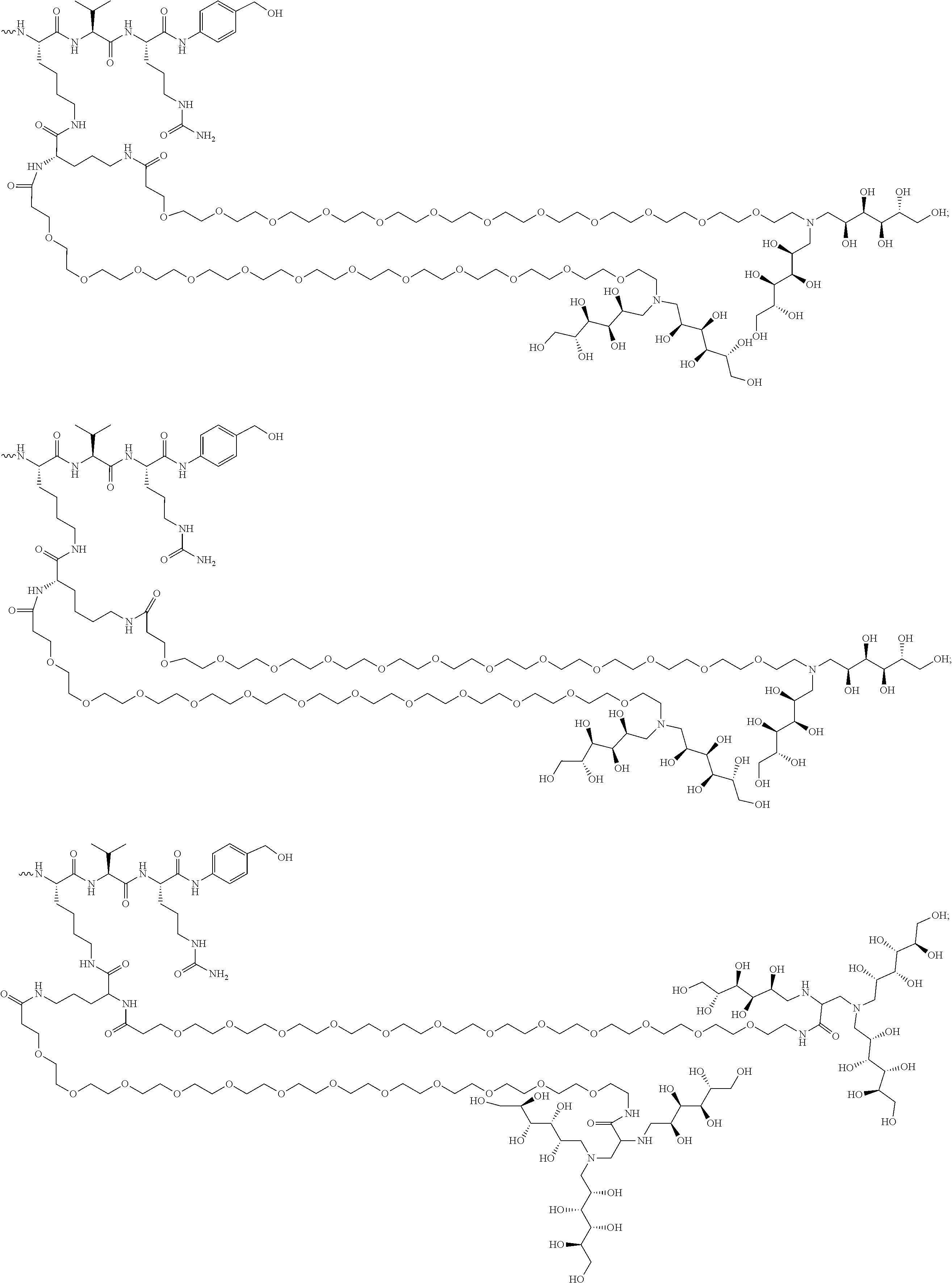

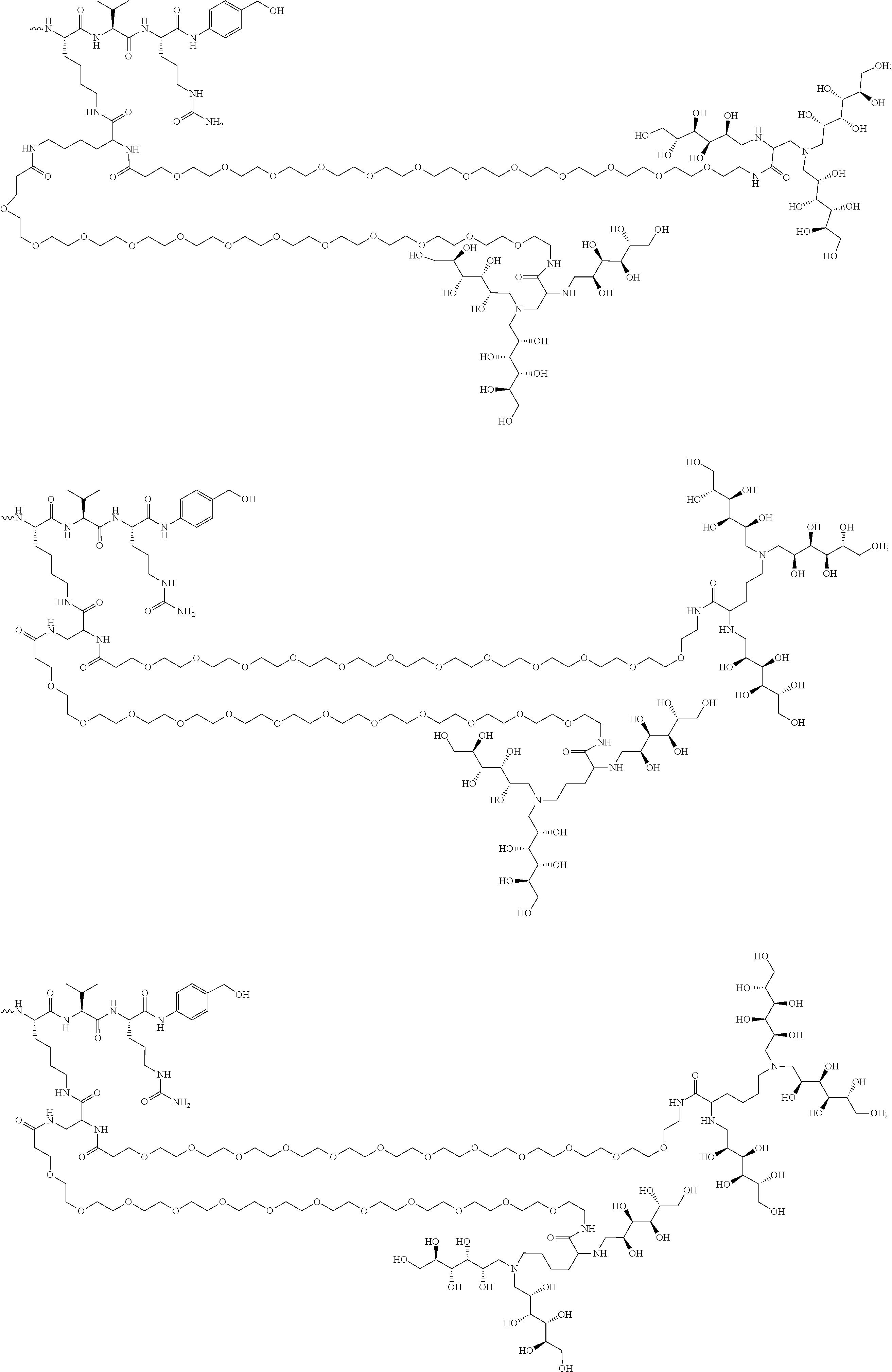

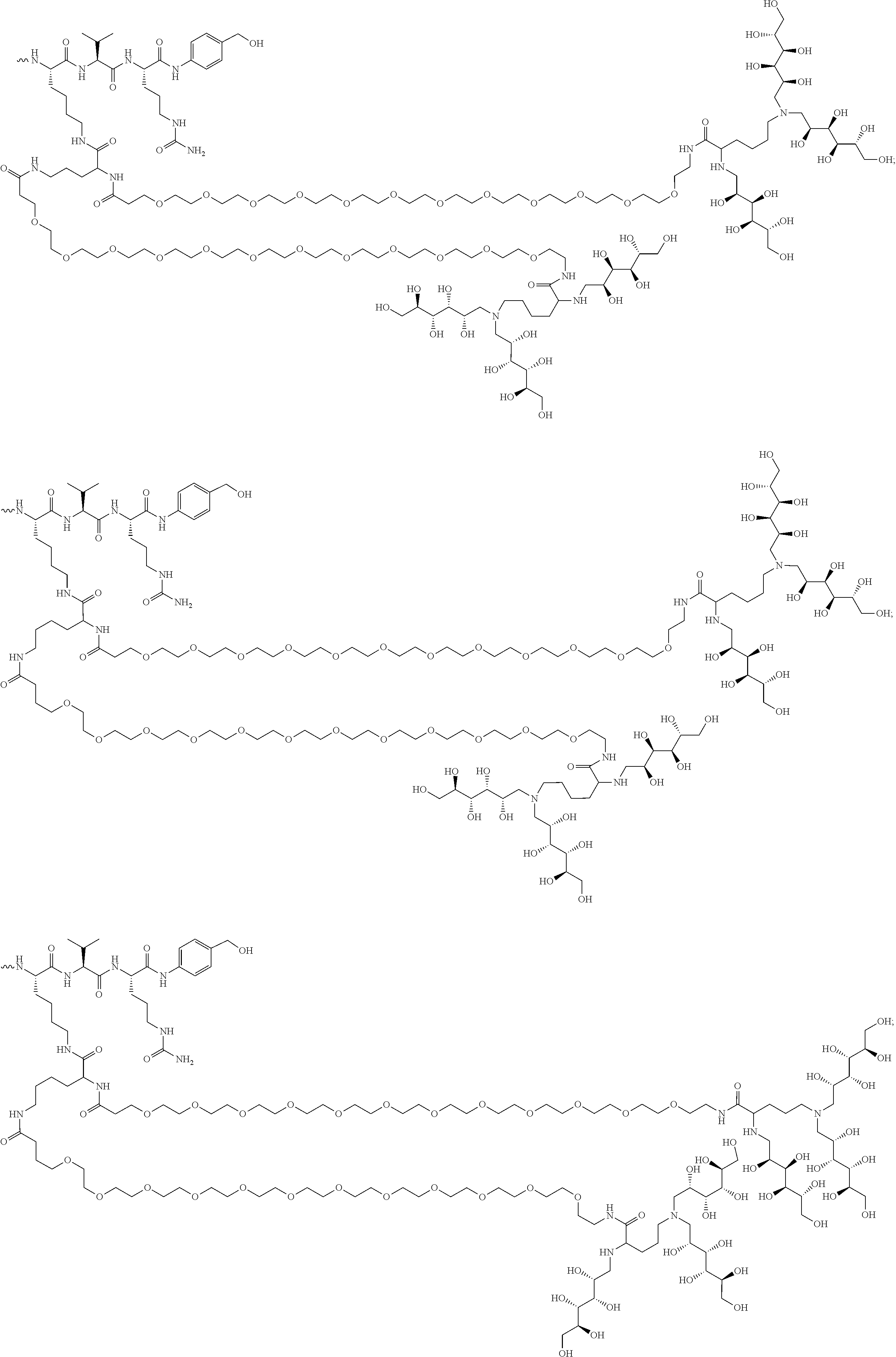

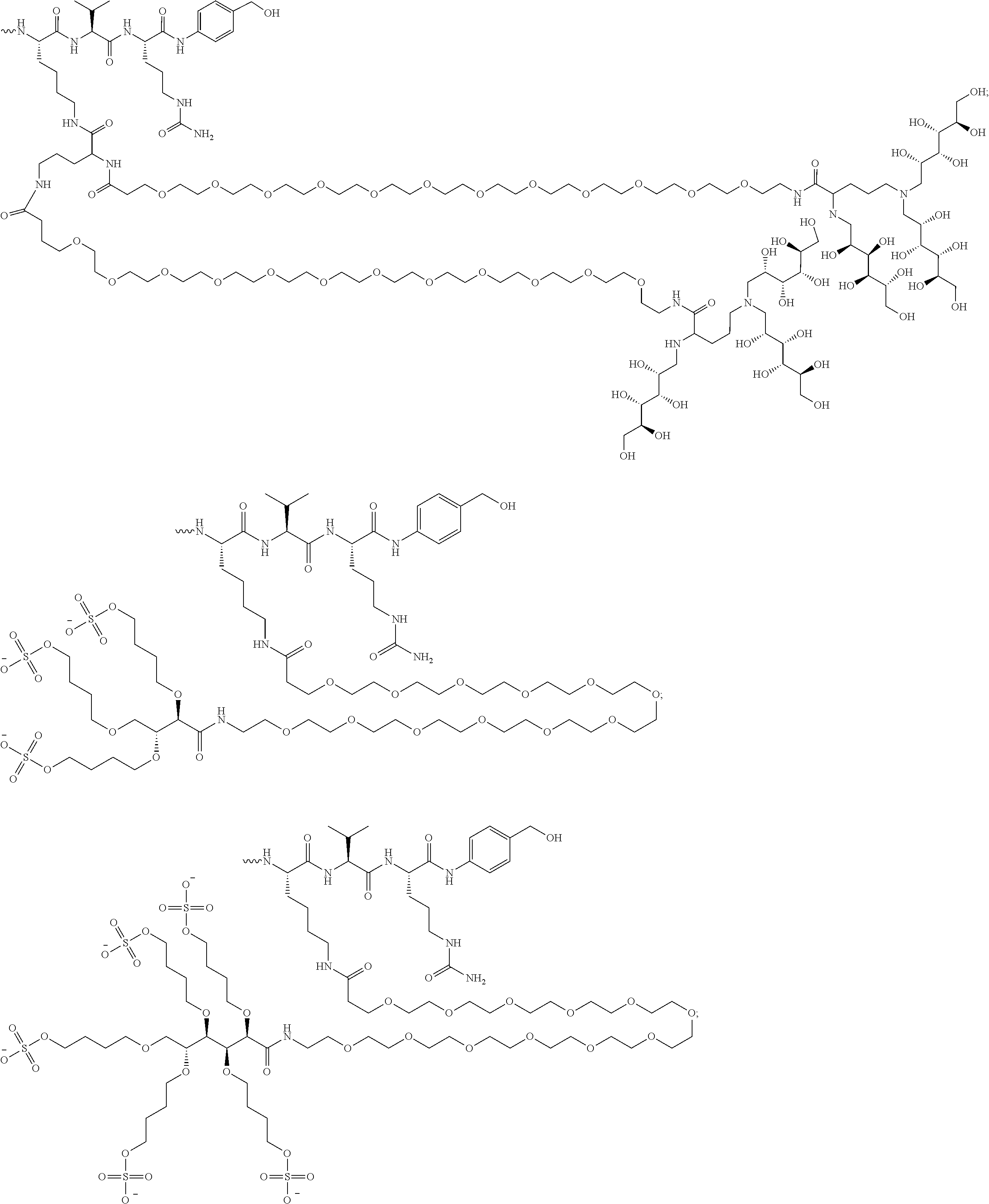

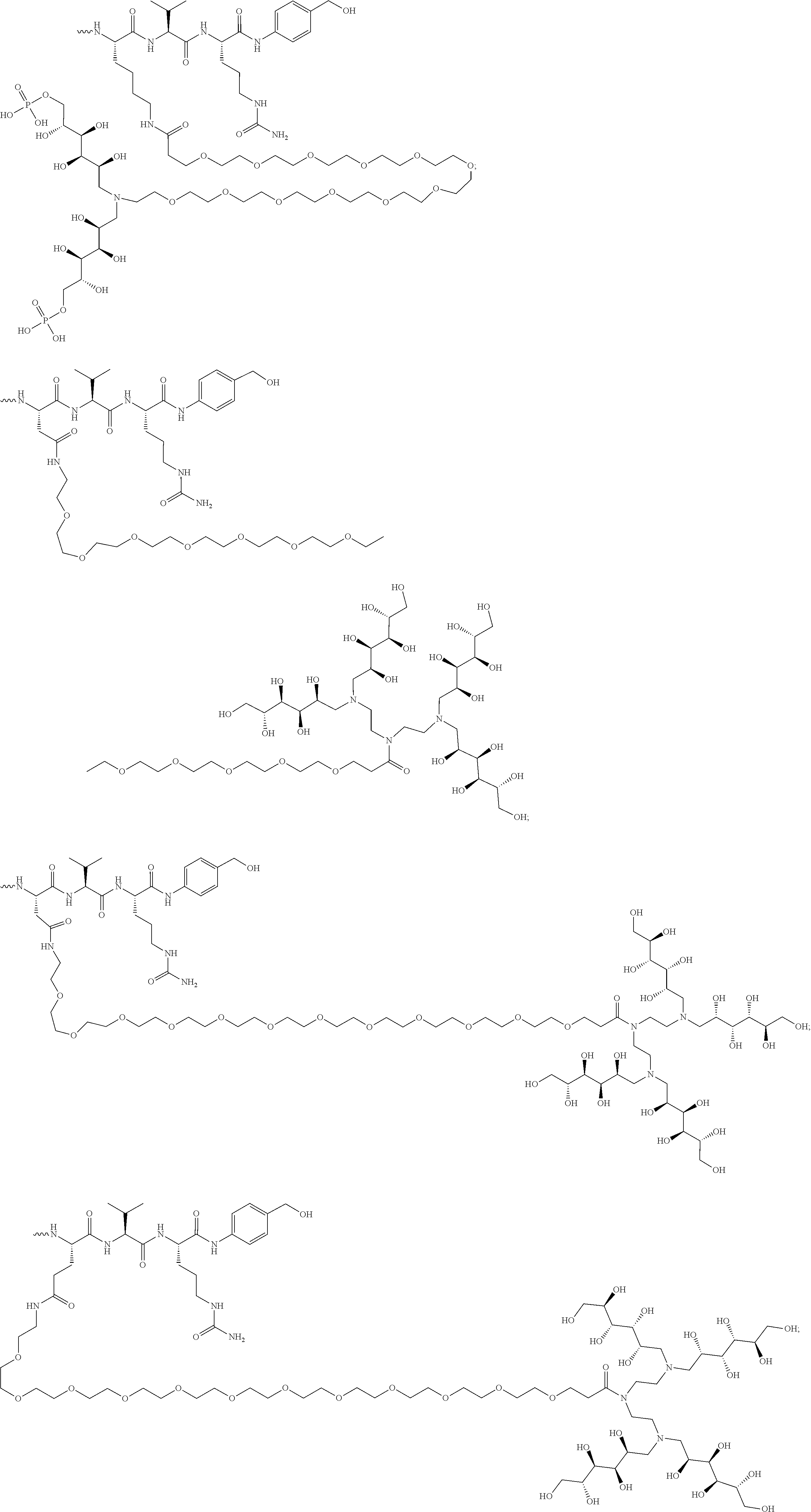

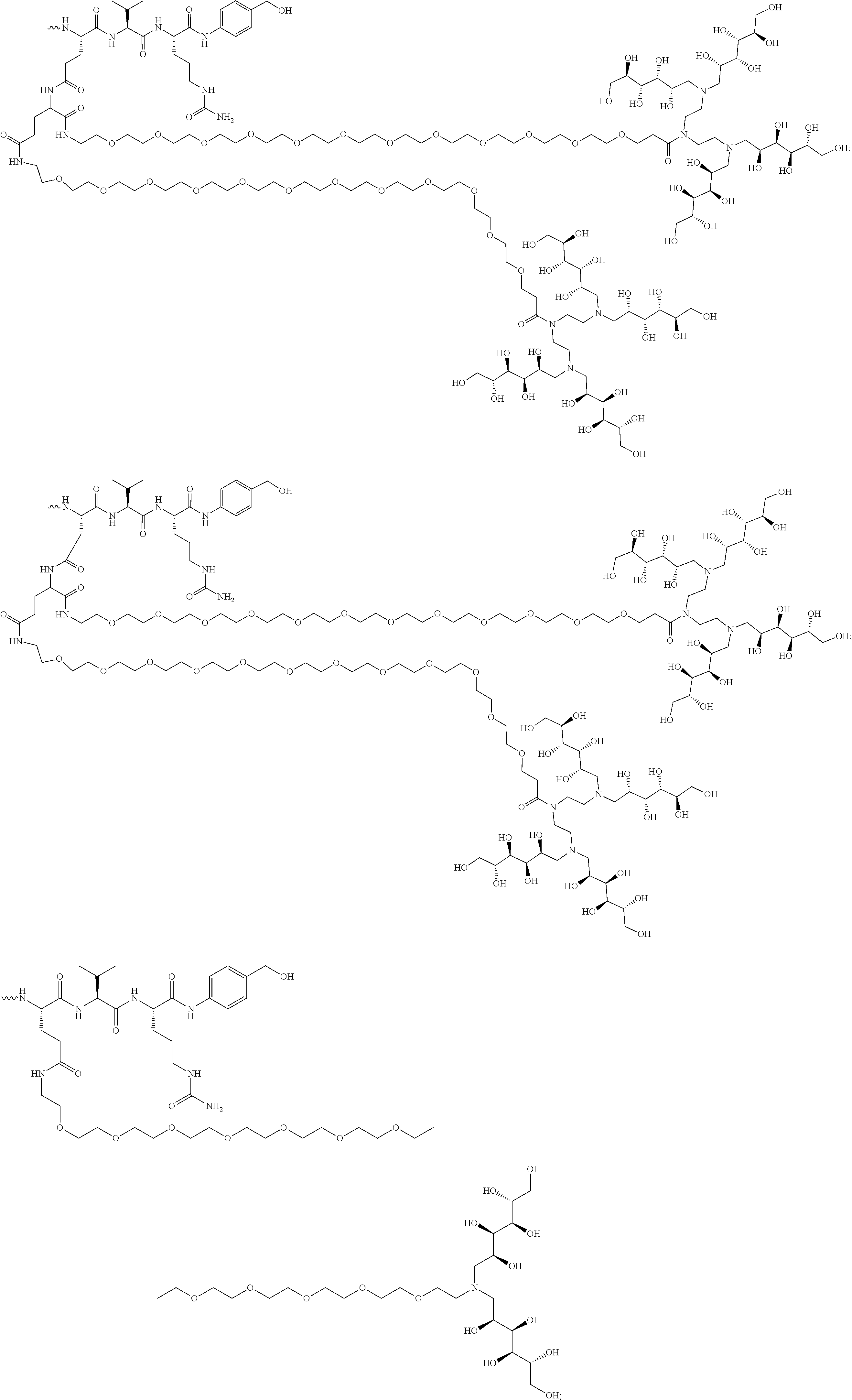

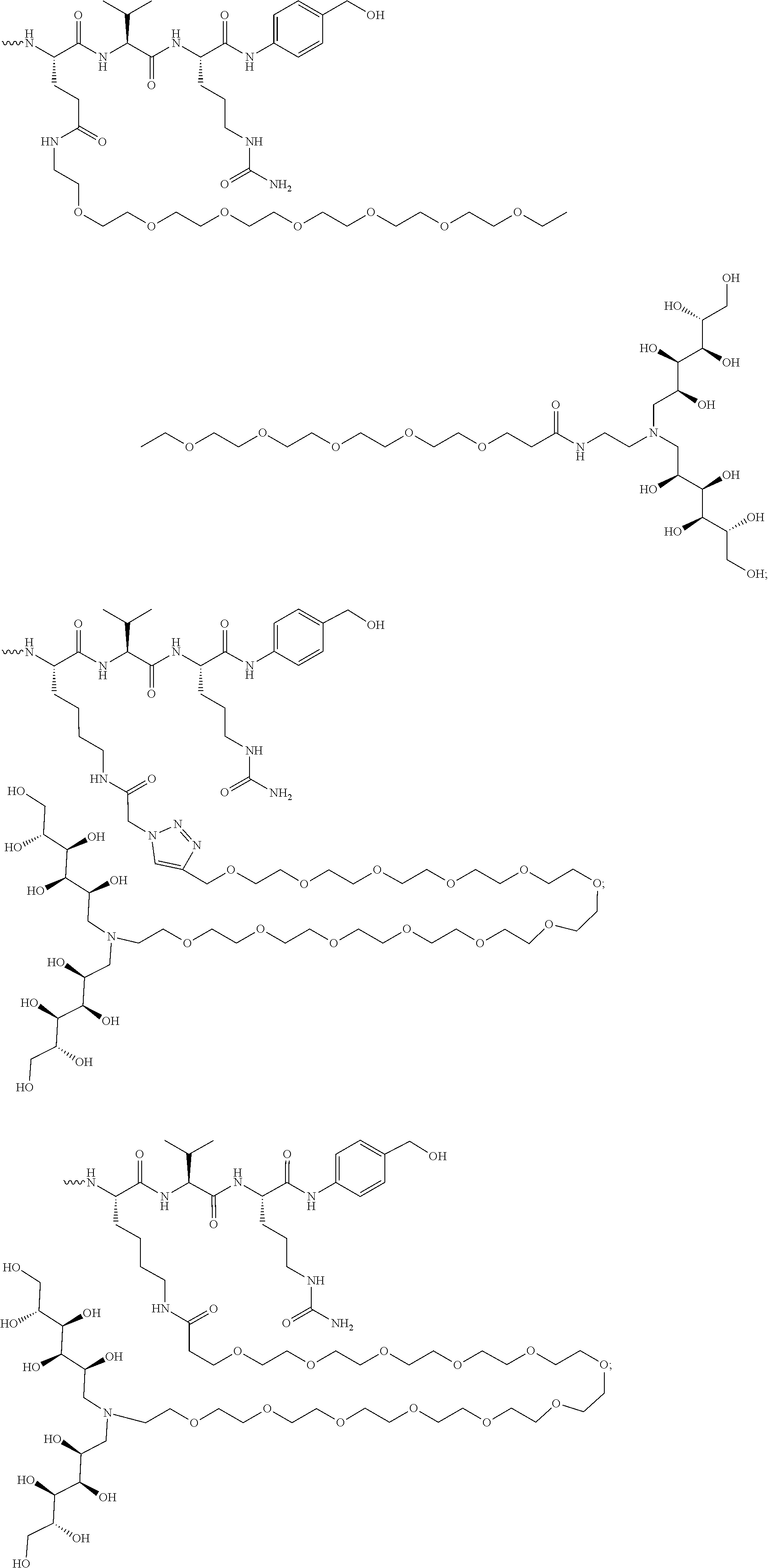

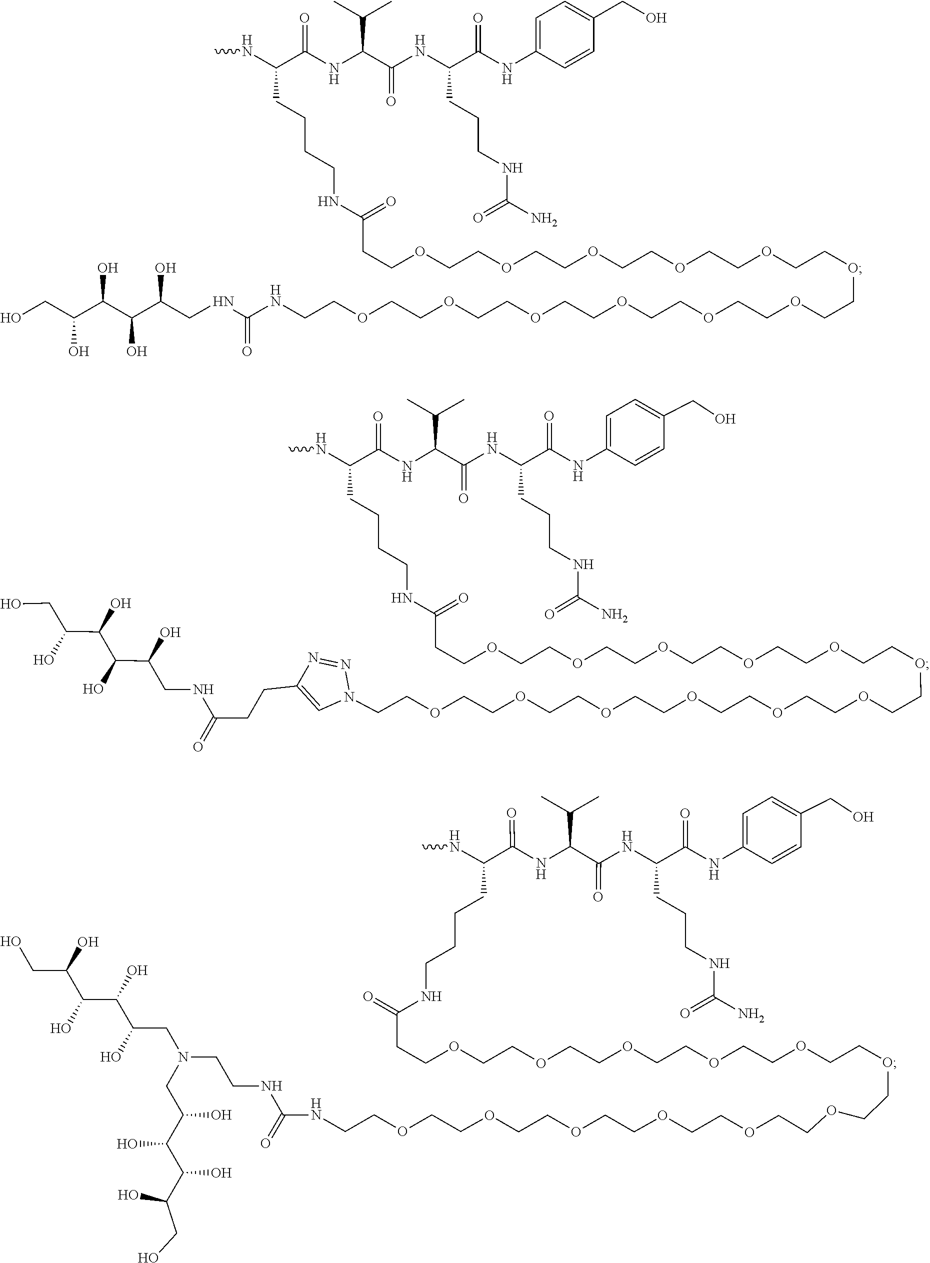

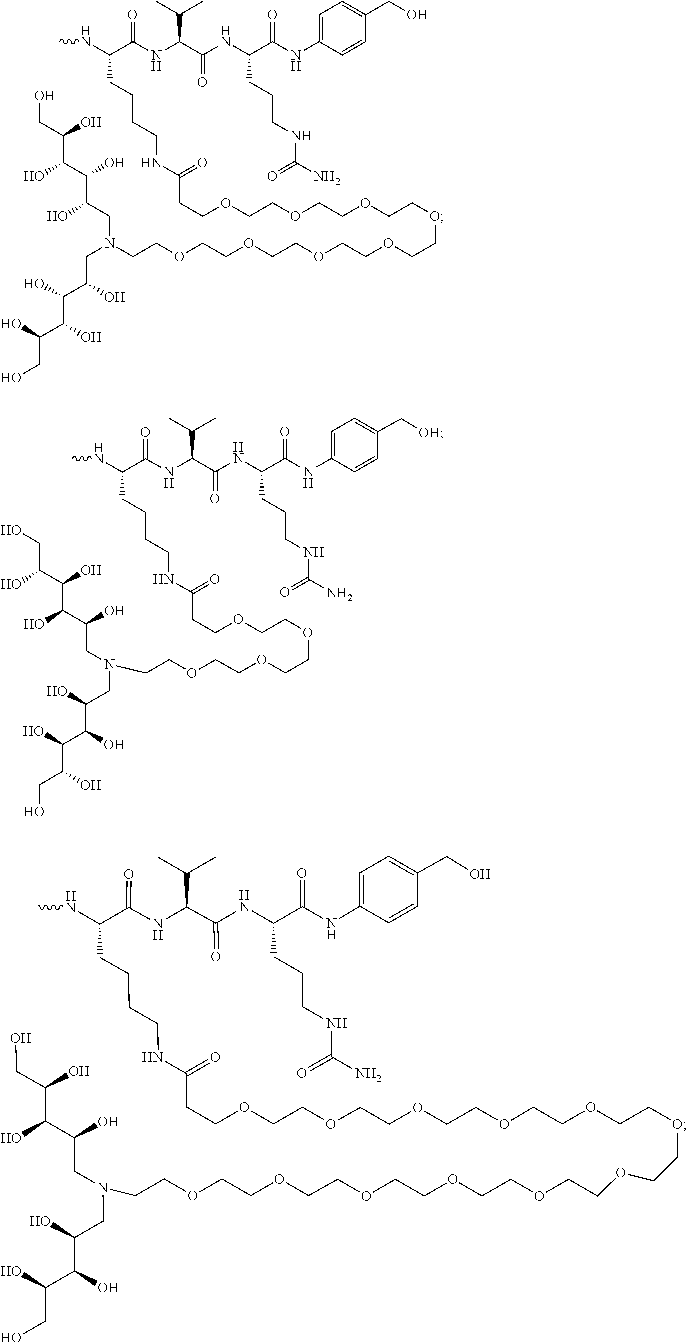

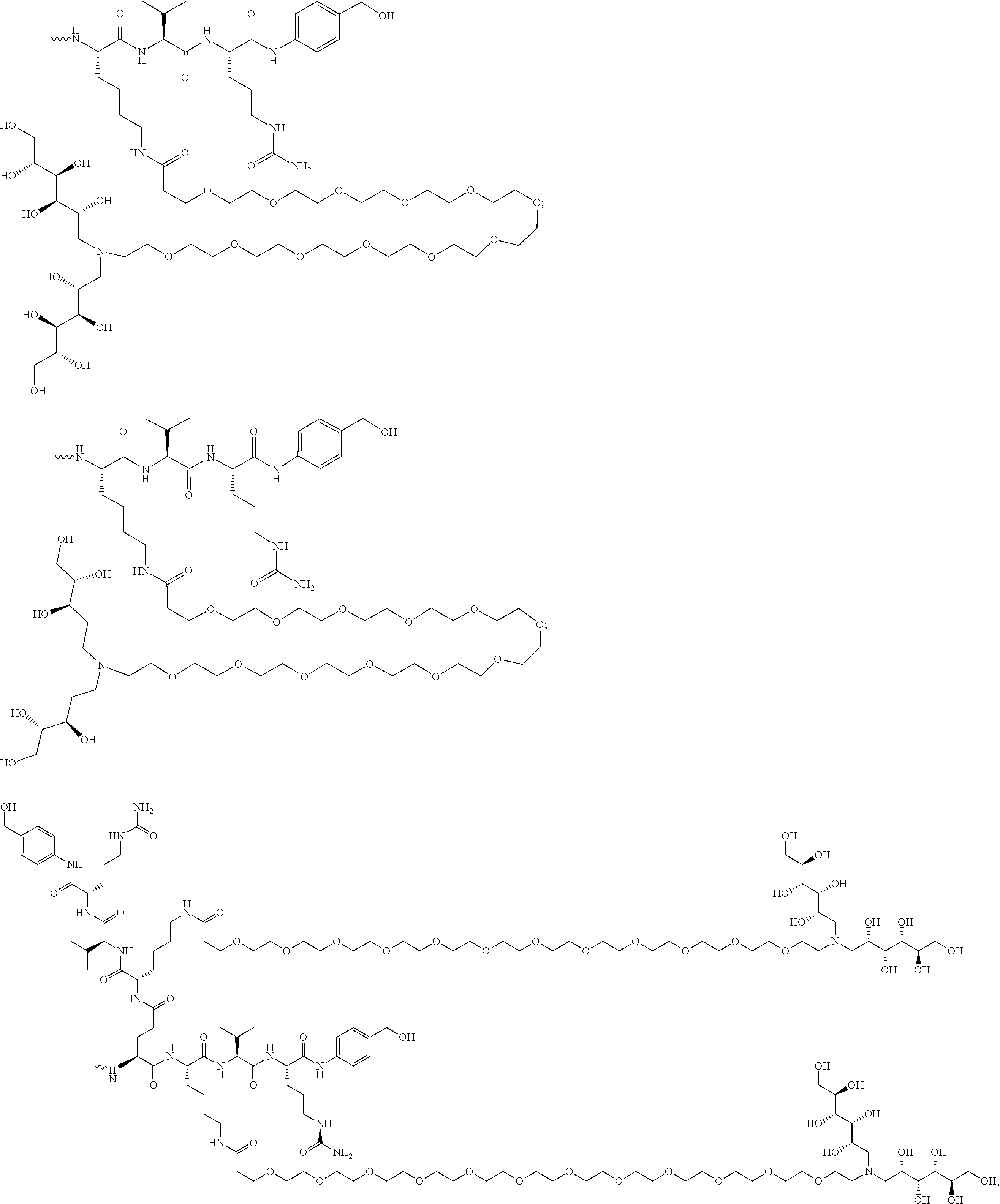

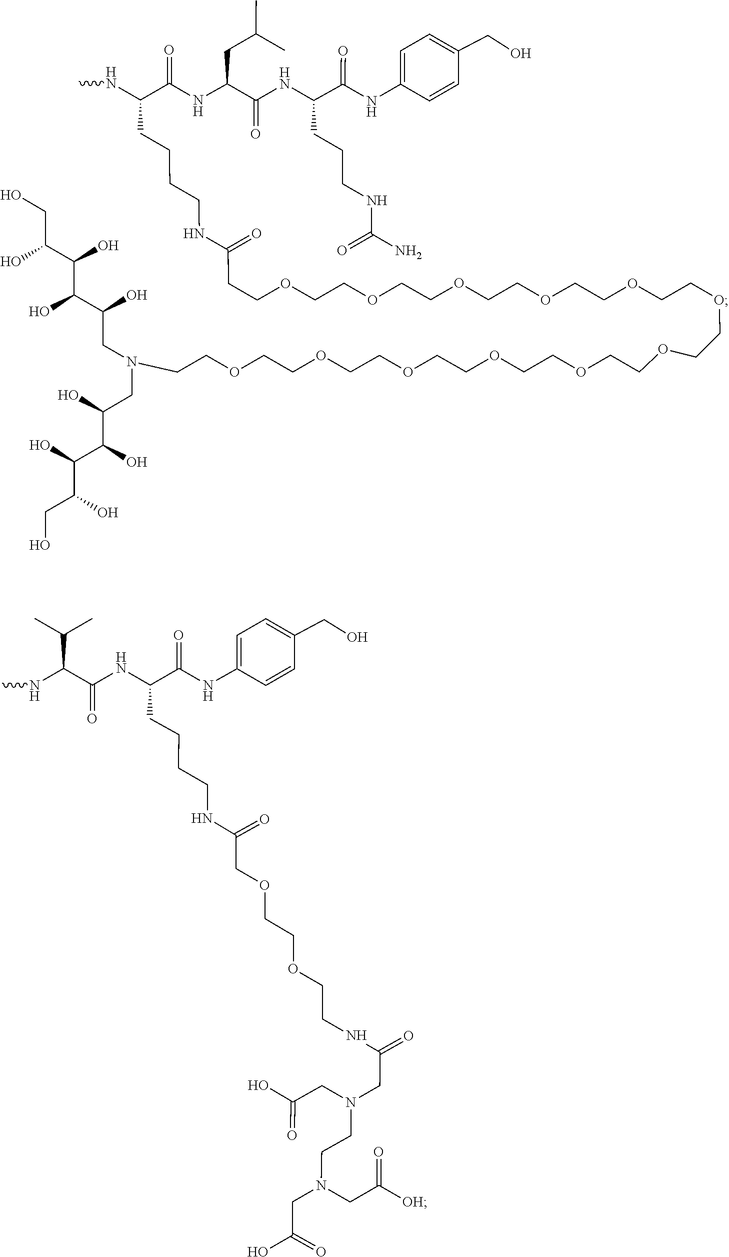

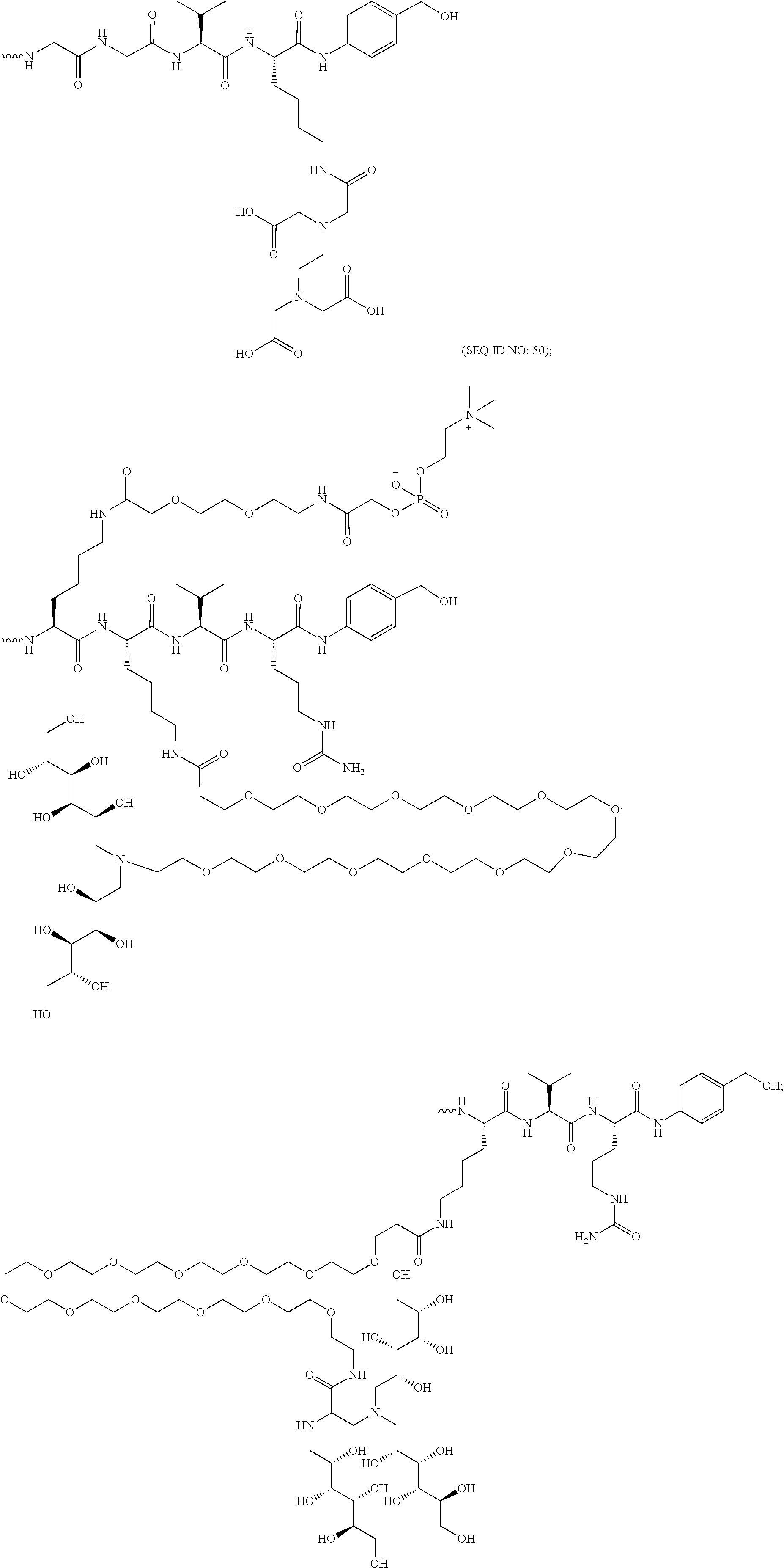

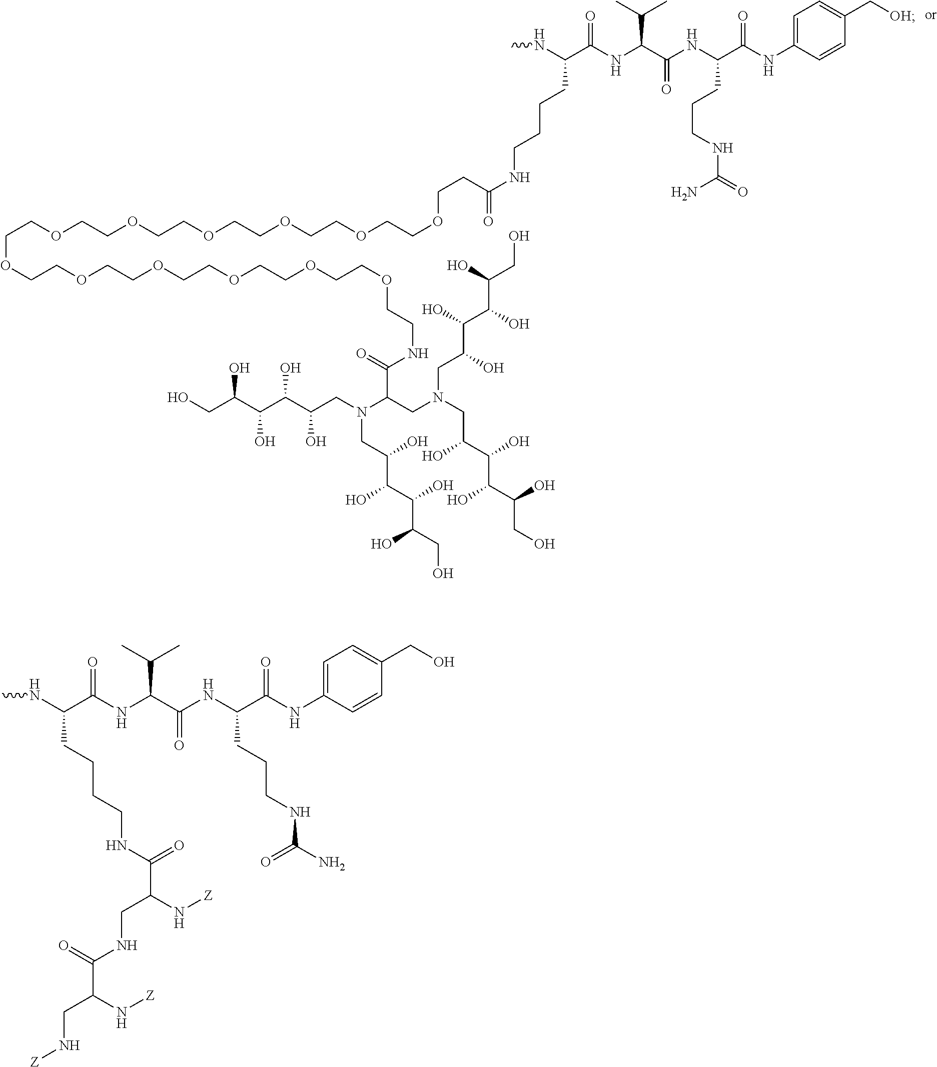

wherein each Z is attached at * and is individually selected from:

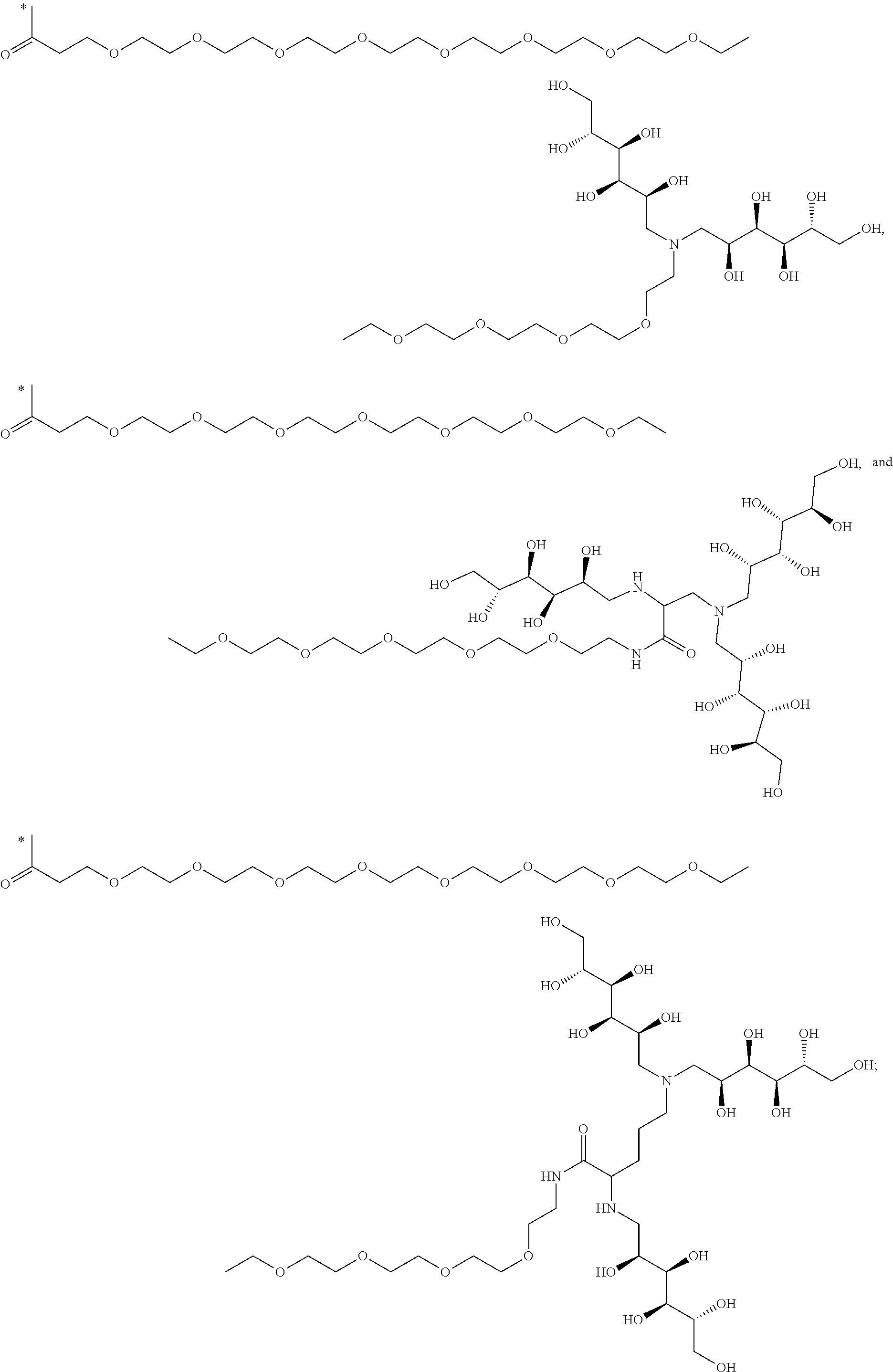

wherein the wavy line on the amino group indicates an attachment site for a Stretcher unit, and the Drug unit is attached to the benzyl alcohol (i.e., the H of benzyl alcohol is replaced with a bond to the Drug unit).

In some embodiments, provided is a Linker intermediate or Linker, wherein L2 is attached to a side chain of a subunit of AA. In some embodiments, provided is a Linker intermediate or Linker, wherein ˜AA-L2≈ has one of the following structures:

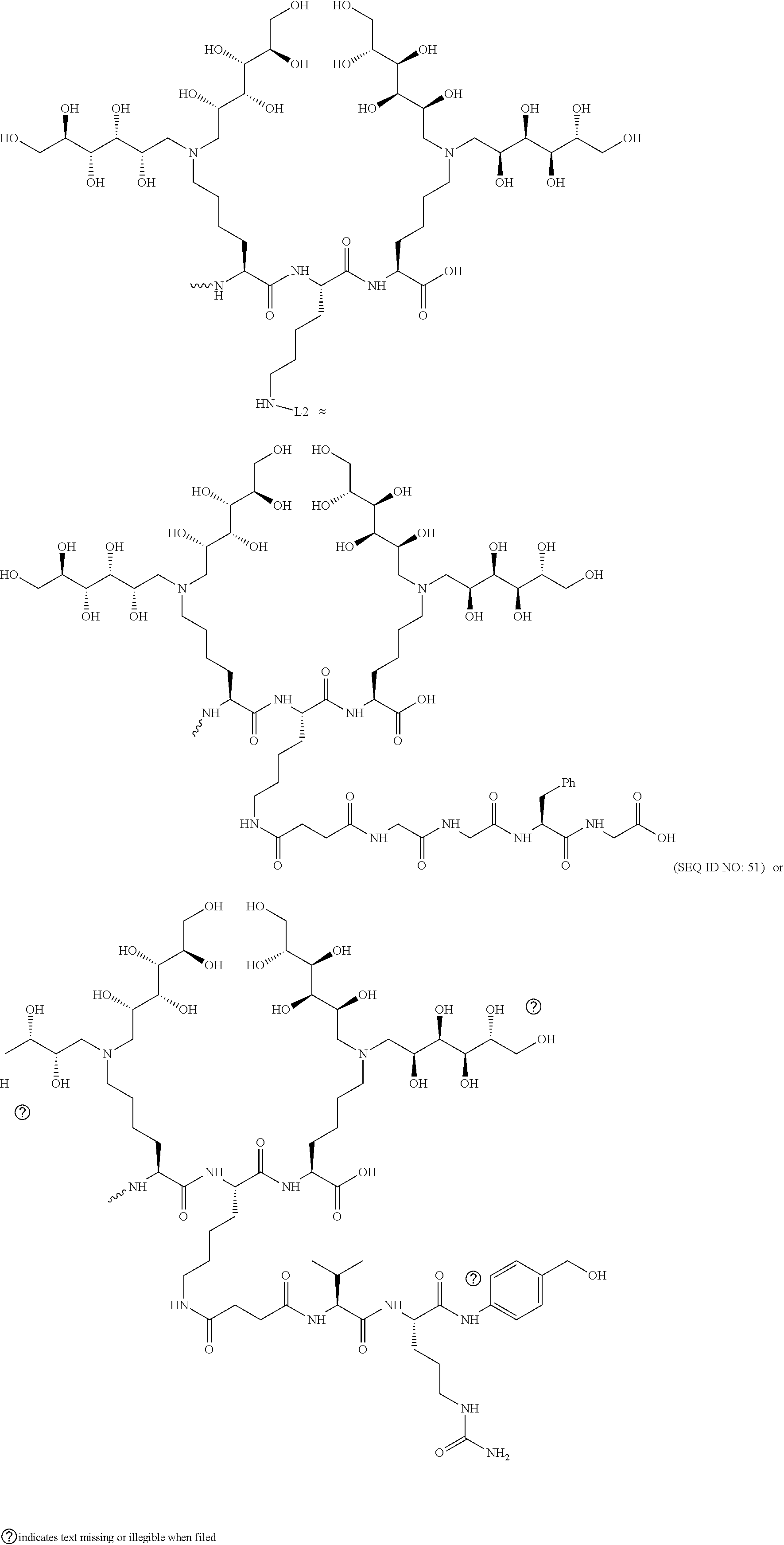

wherein the wavy line on the amino group indicates an attachment site for a Stretcher unit, and the Drug unit is attached to the terminal acid group or the benzyl alcohol (i.e., the H of the acid or benzyl alcohol is replaced with a bond to the Drug unit), or wherein the wavy (˜) line indicates an attachment site for the Drug Unit.

In some embodiments, provided is a Linker intermediate or Linker, wherein the Amino Acid unit is joined to Linker Subunit L2 by a non-peptidic linking group. In some embodiments, provided is a Linker intermediate or Linker, wherein the non-peptidic linking group is selected from C₁-C₁₀ alkylene, C₂-C₁₀ alkenylene, C₂-C₁₀ alkynylene, or polyethylene glycol.

In some embodiments, provided is a Linker intermediate or Linker, further comprising a Stretcher unit to from a Linker. In some embodiments, provided is a Linker, wherein the Stretcher unit is selected from the following:

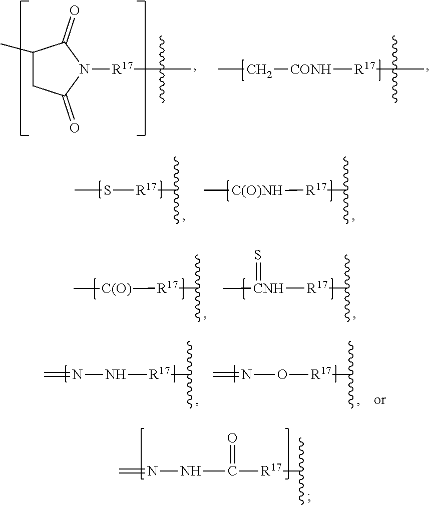

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene-(where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene-(where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)—NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)—NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the Stretcher unit comprises maleimido(C₁-C₁₀alkylene-C(O)—, maleimido(CH₂OCH₂)ₚ₂(C₁-C₁₀alkyene)C(O)—, maleimido(C₁-C₁₀alkyene)(CH₂OCH₂)ₚ₂C(O)—, or a ring open form thereof, wherein p2 is from 1 to 26.

In some embodiments, provided is a Linker, wherein the Stretcher unit is selected from the following:

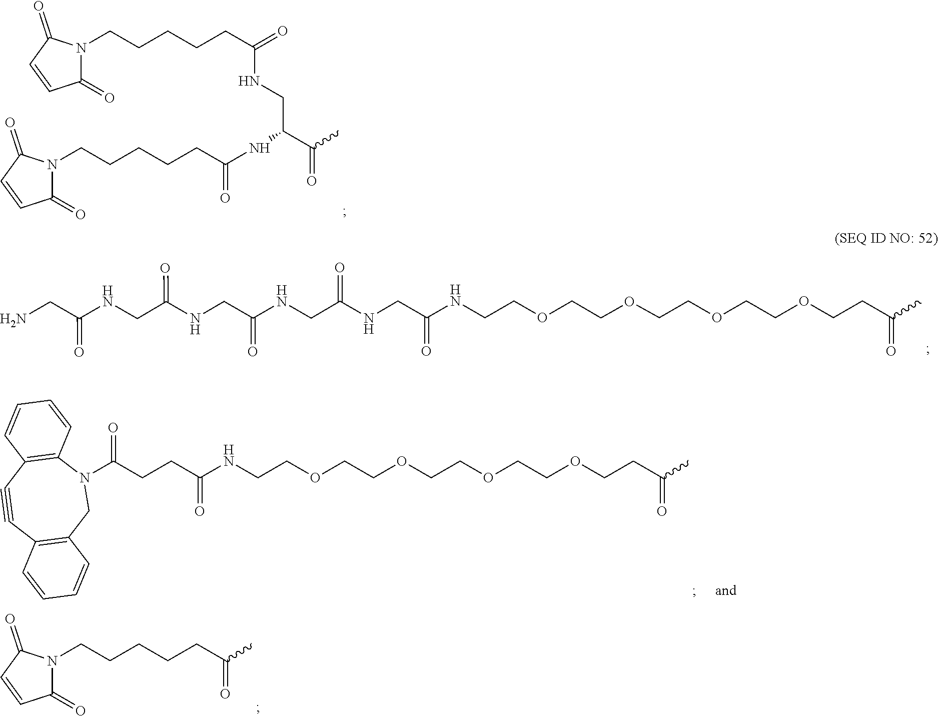

wherein the wavy line [symbol] indicates an attachment site of the Stretcher unit to an Amino Acid unit, and the attachment site to the Targeting unit is on a maleimide, primary amine or alkyne functional group.

In some embodiments, provided is a Linker having one of the following structures:

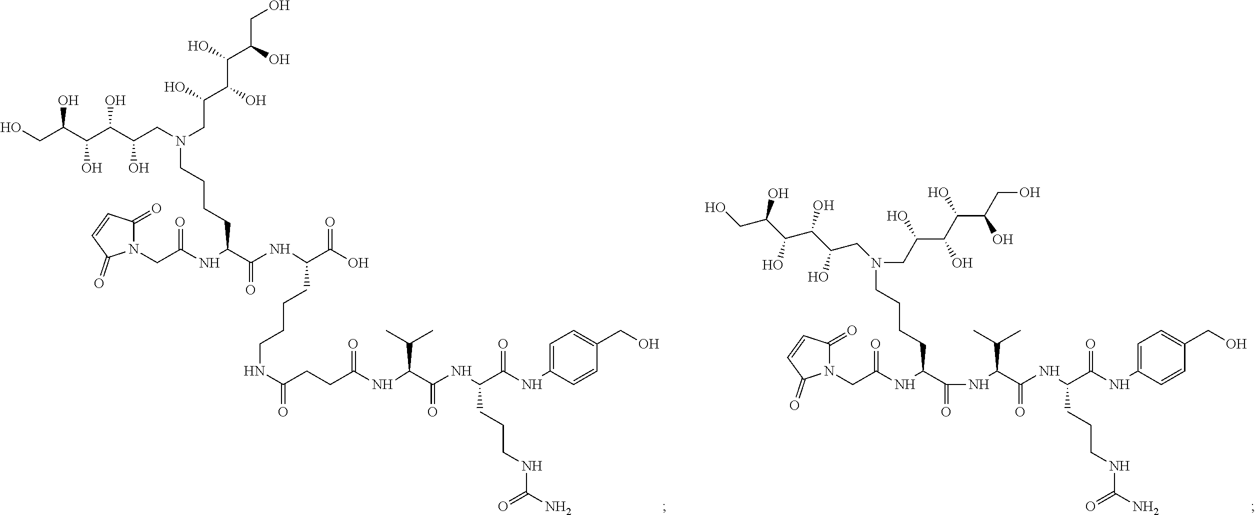

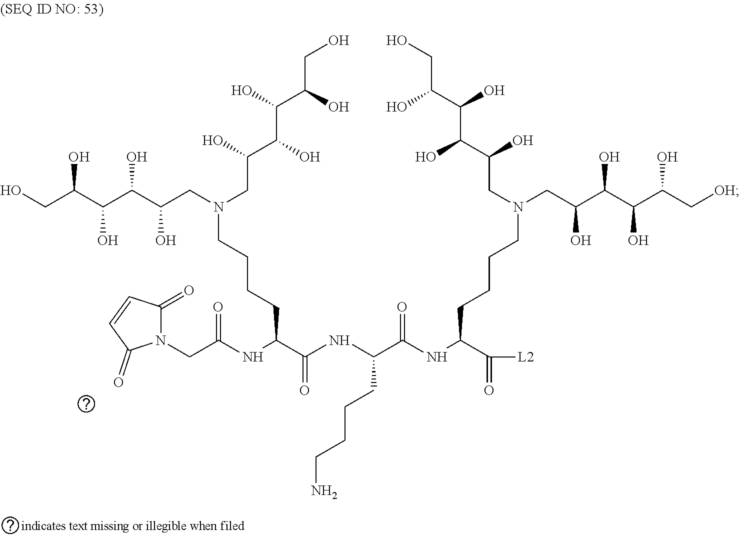

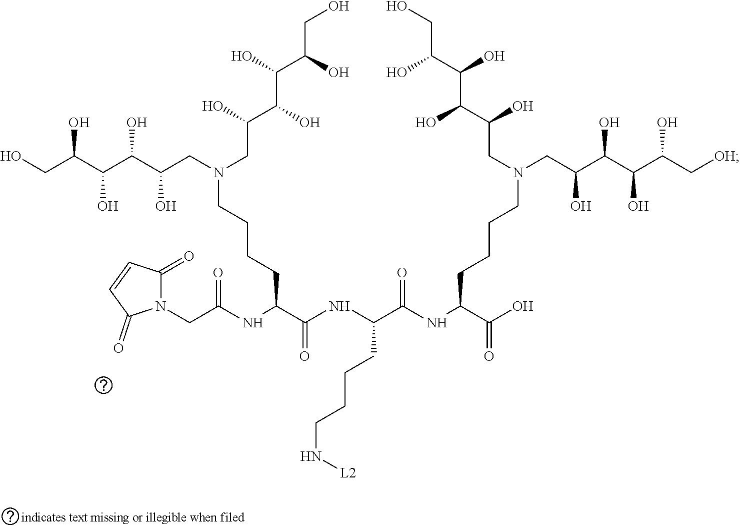

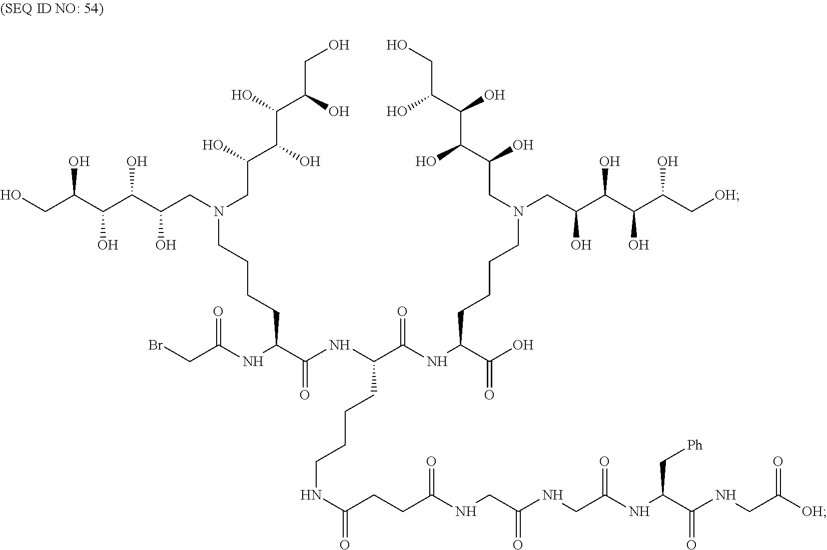

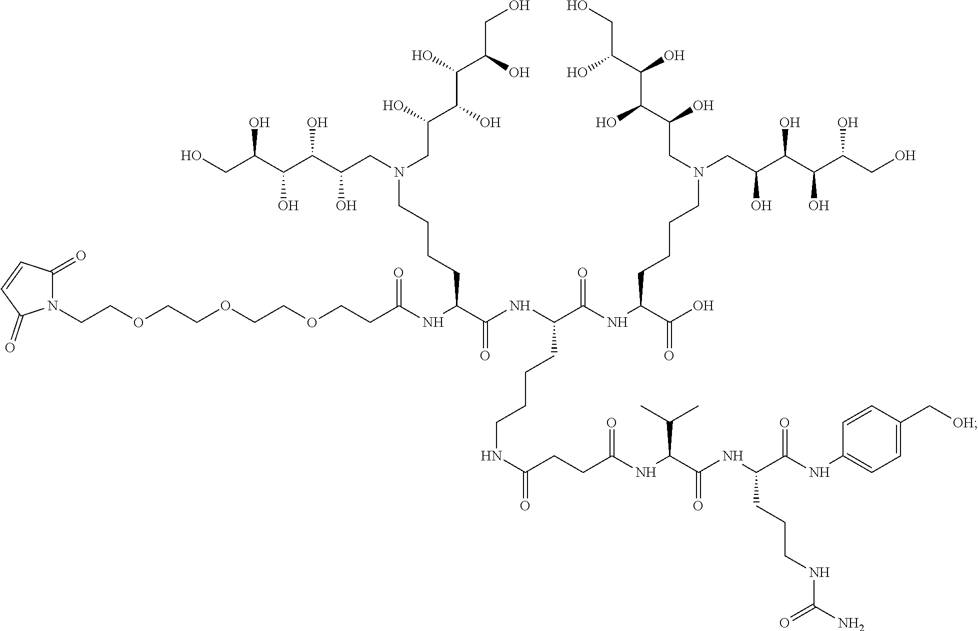

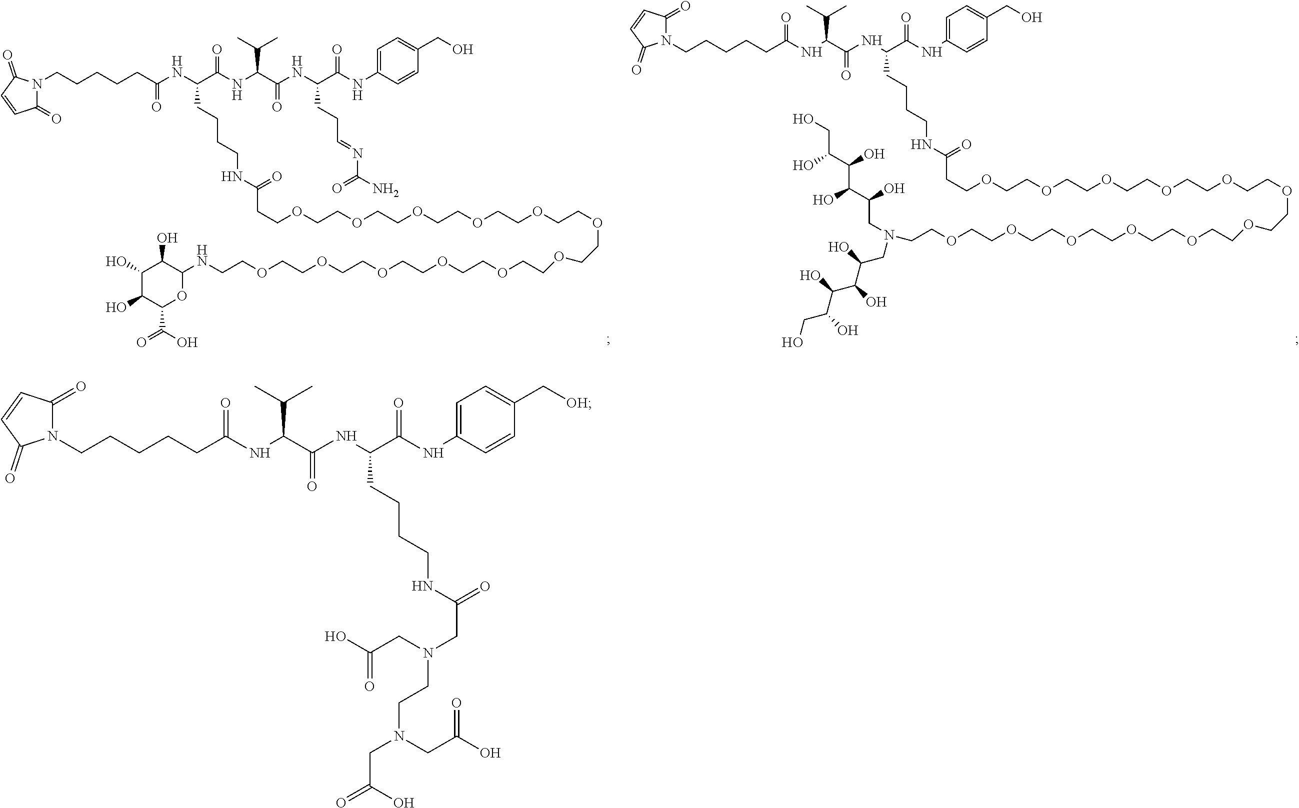

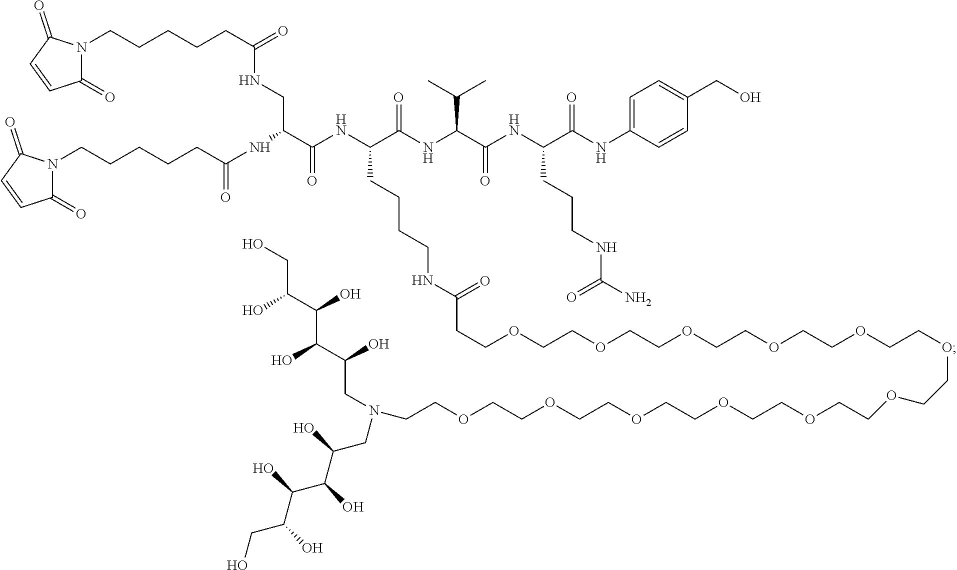

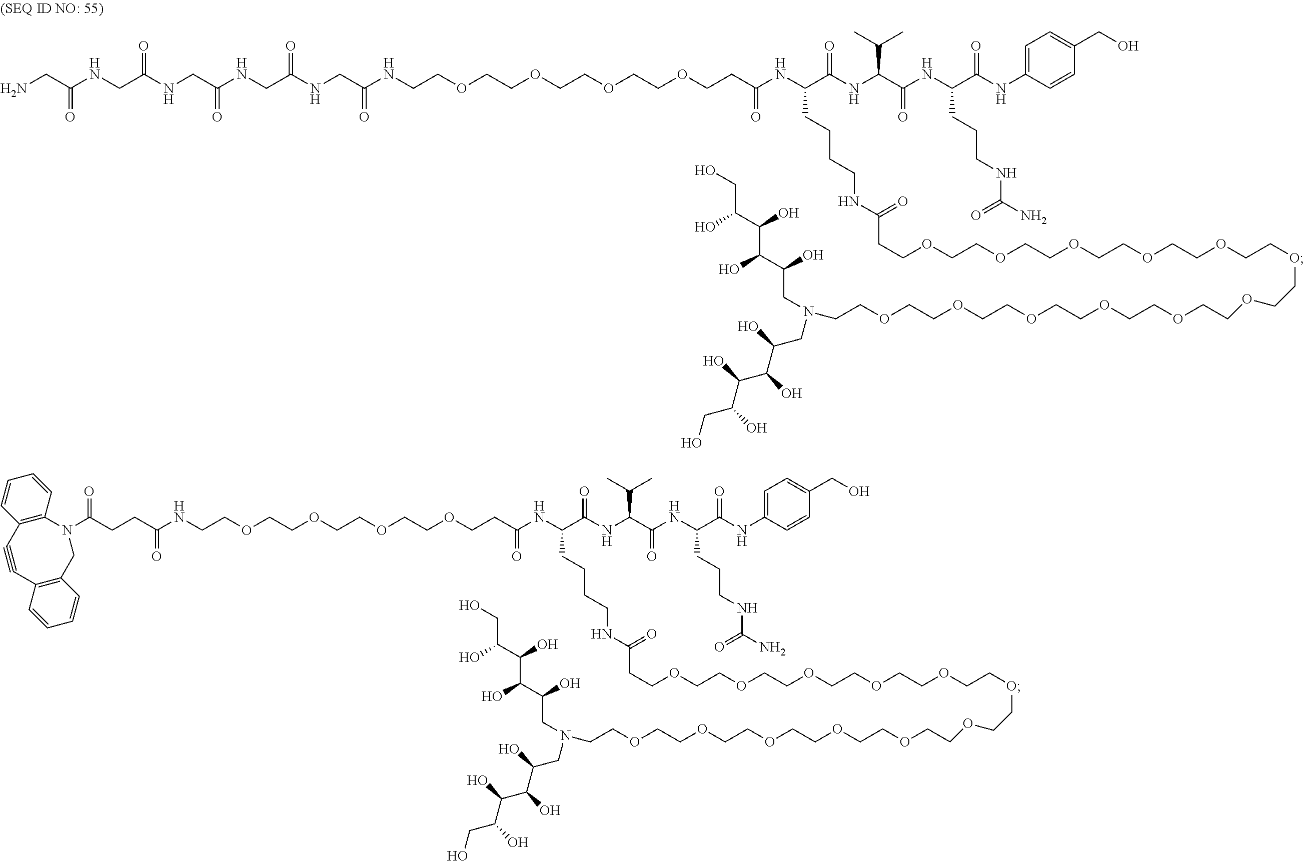

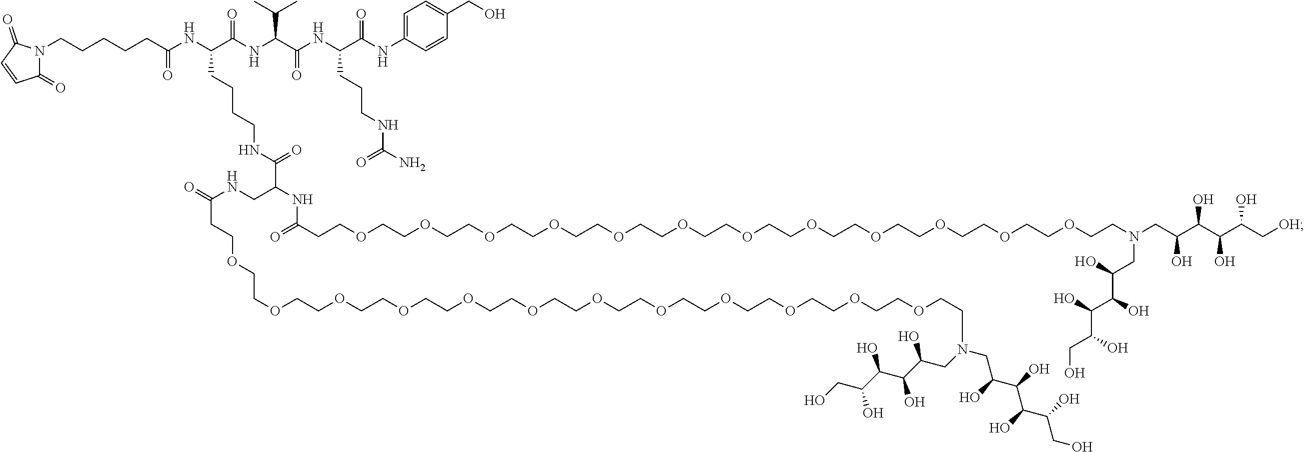

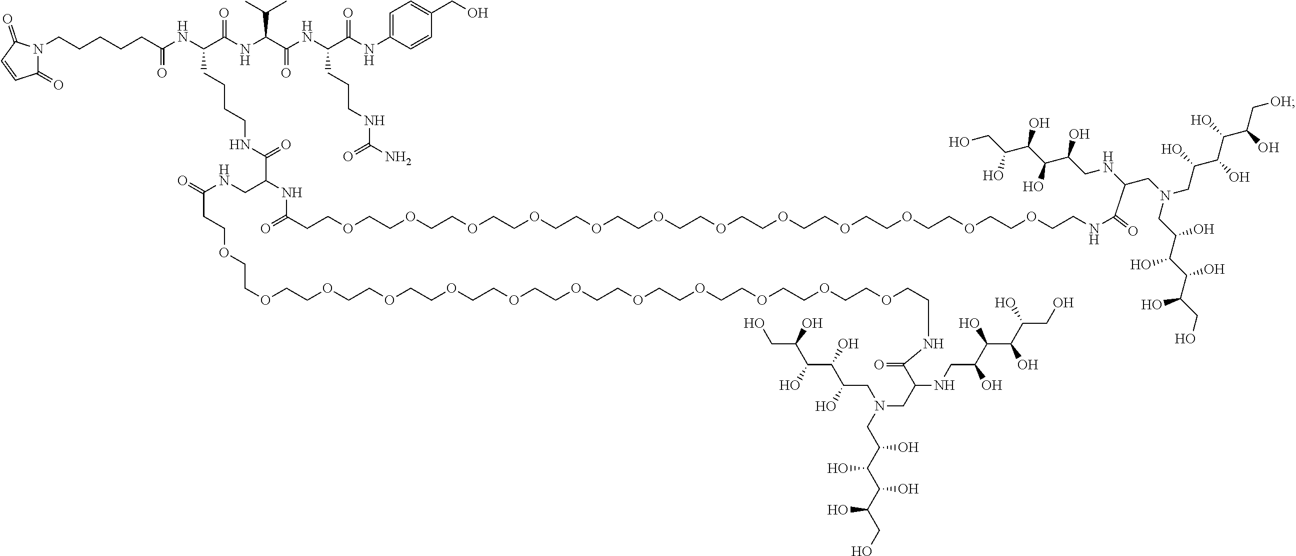

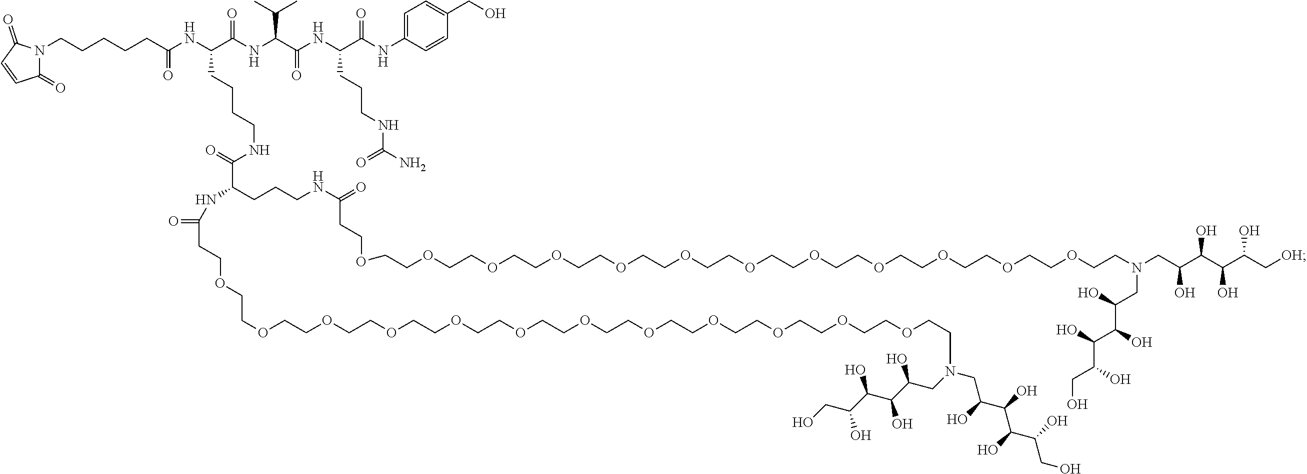

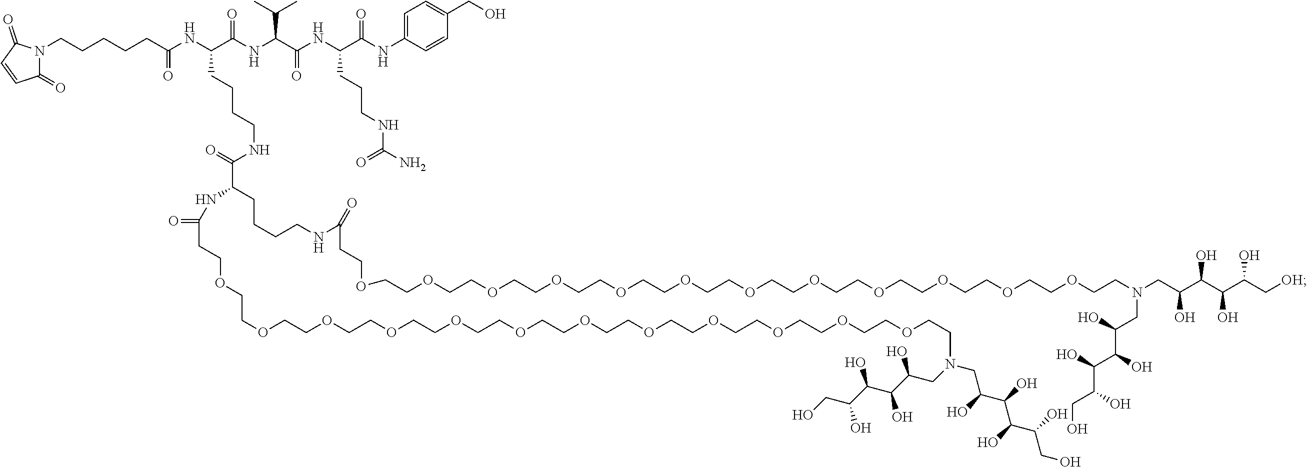

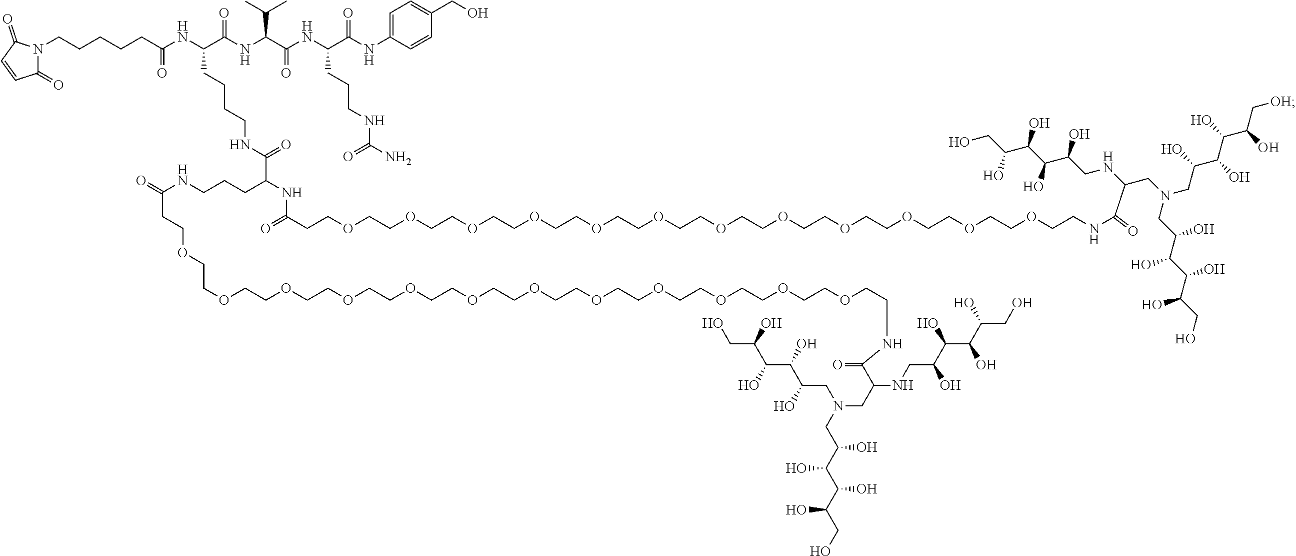

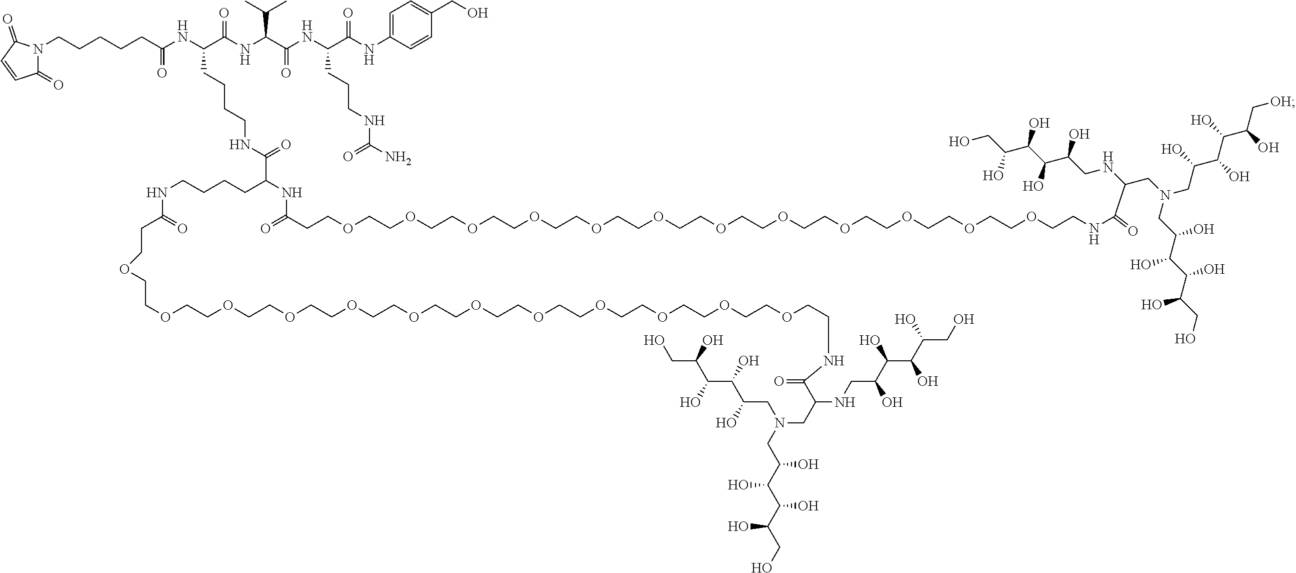

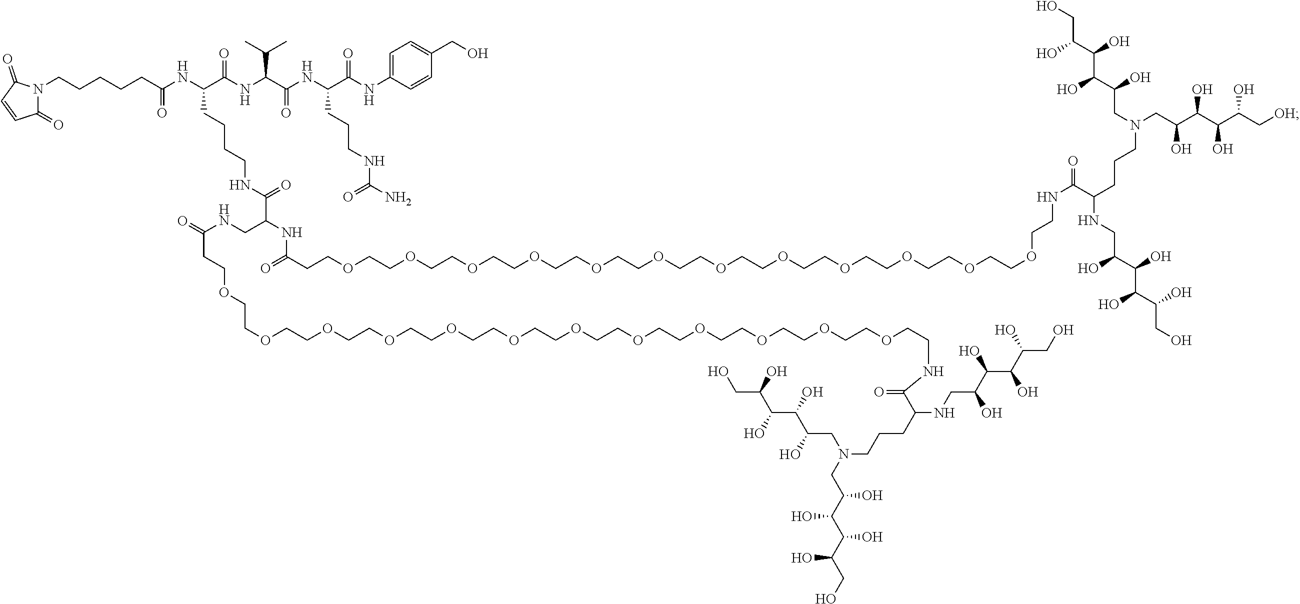

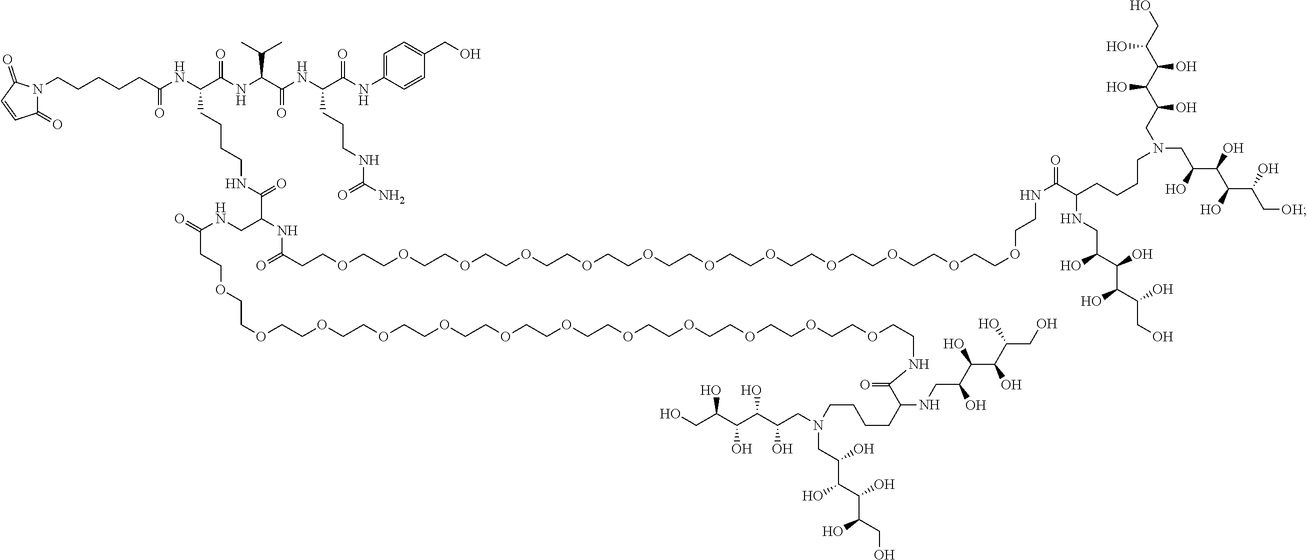

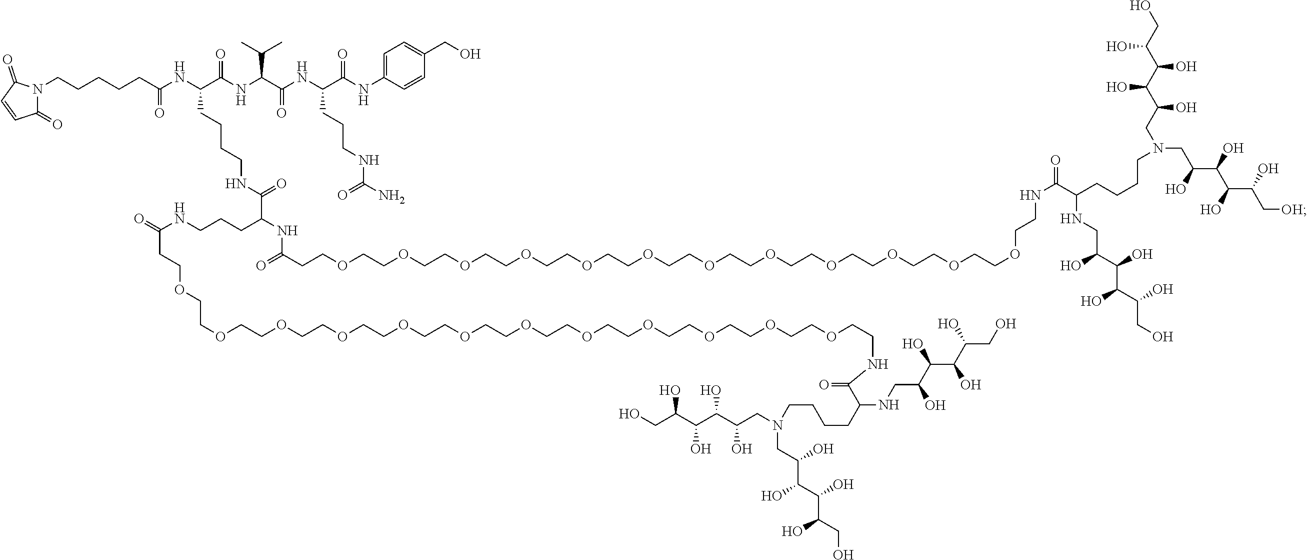

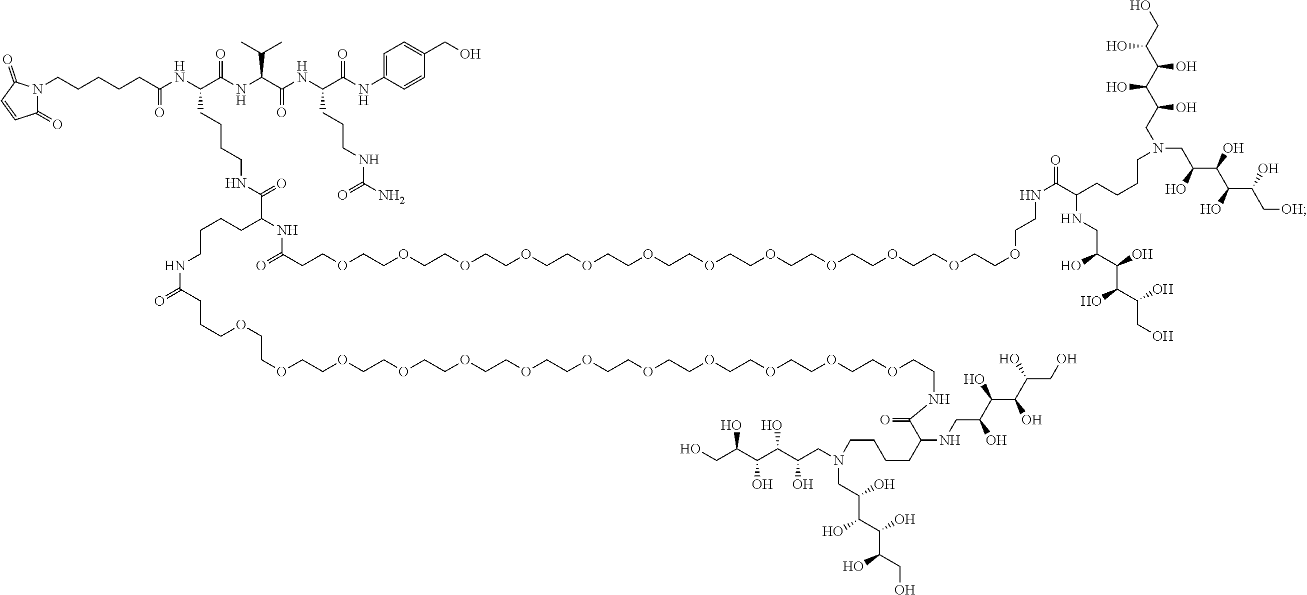

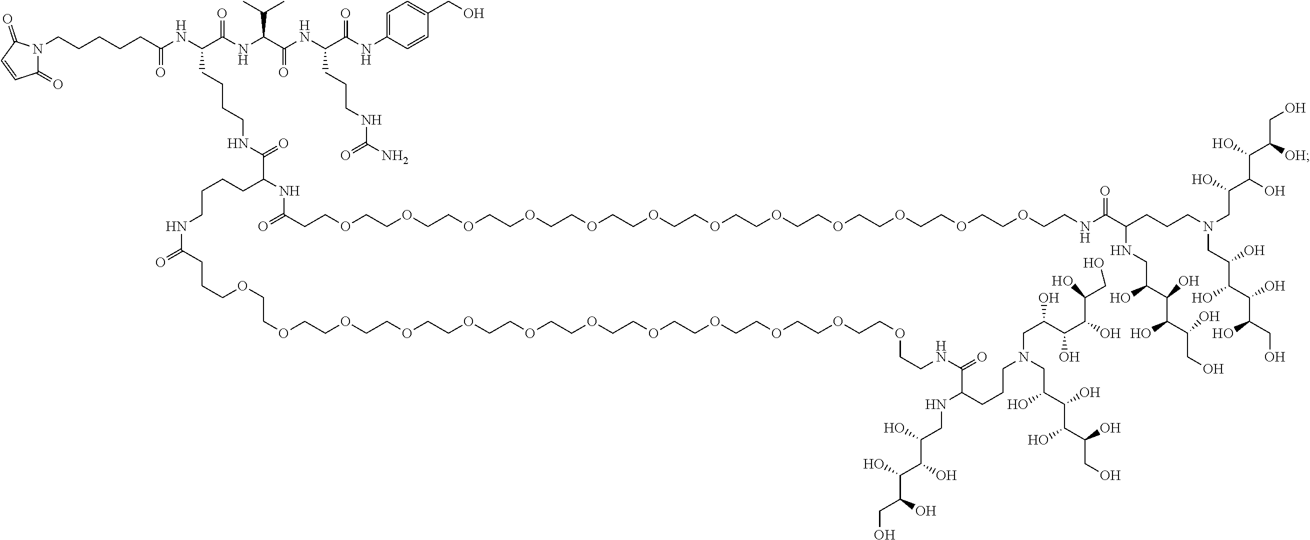

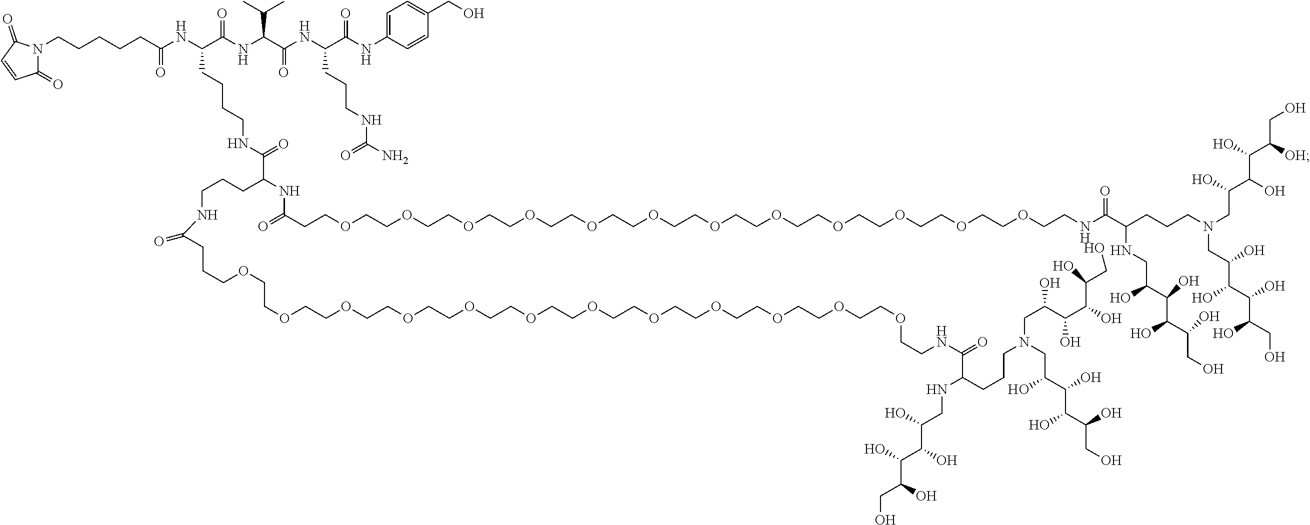

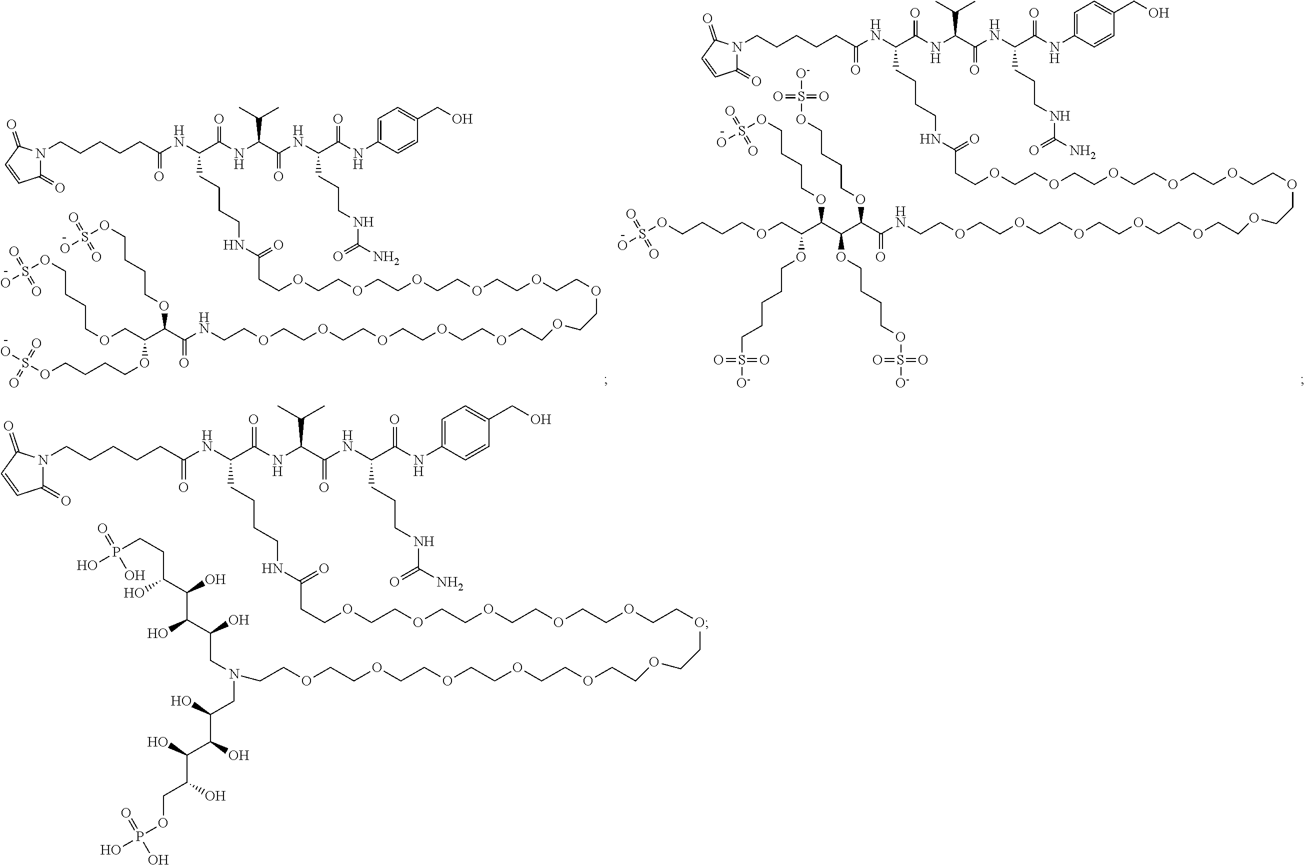

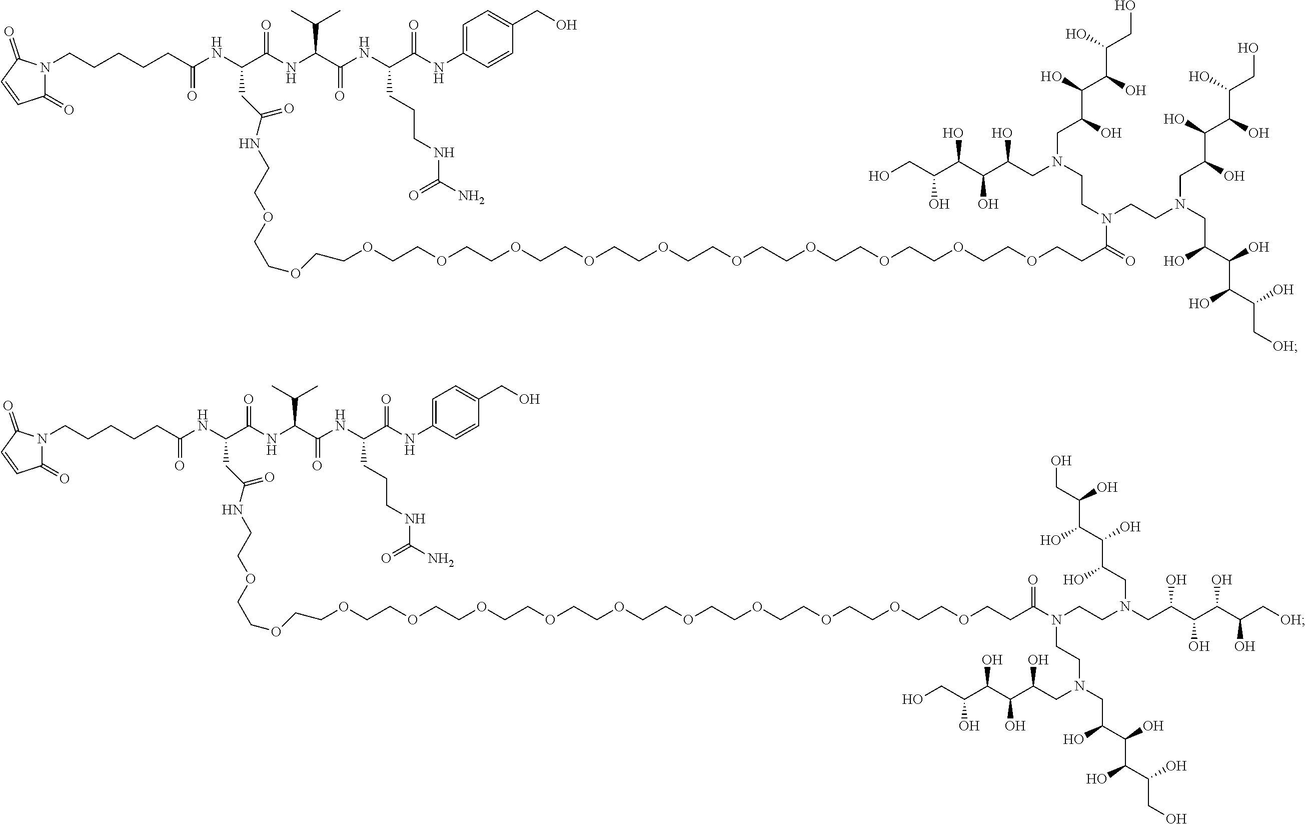

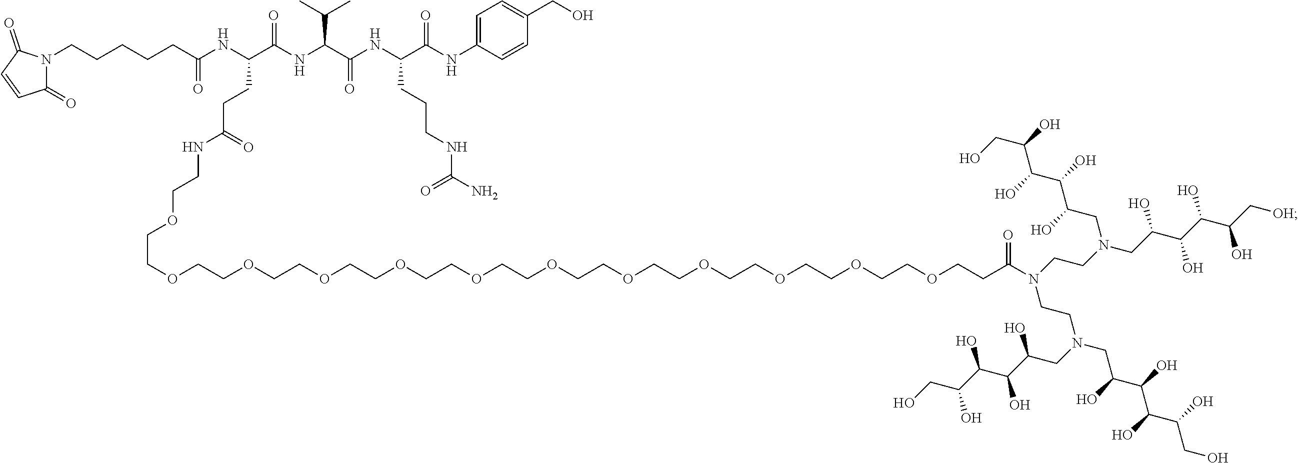

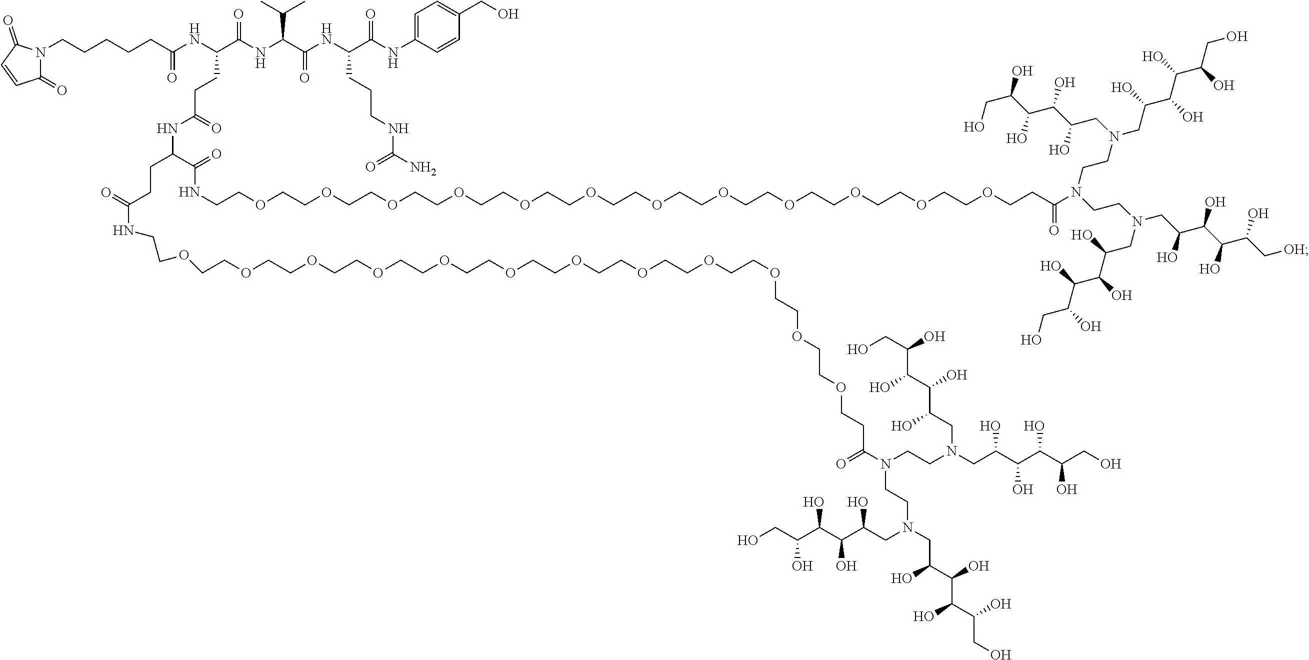

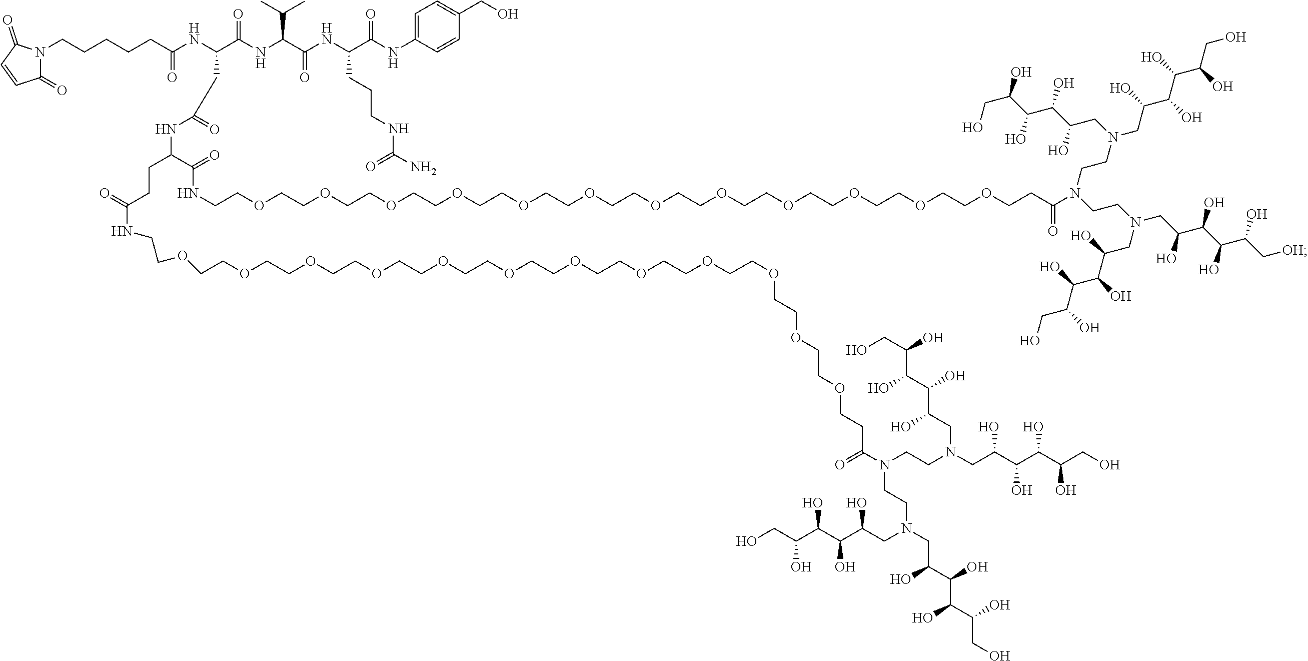

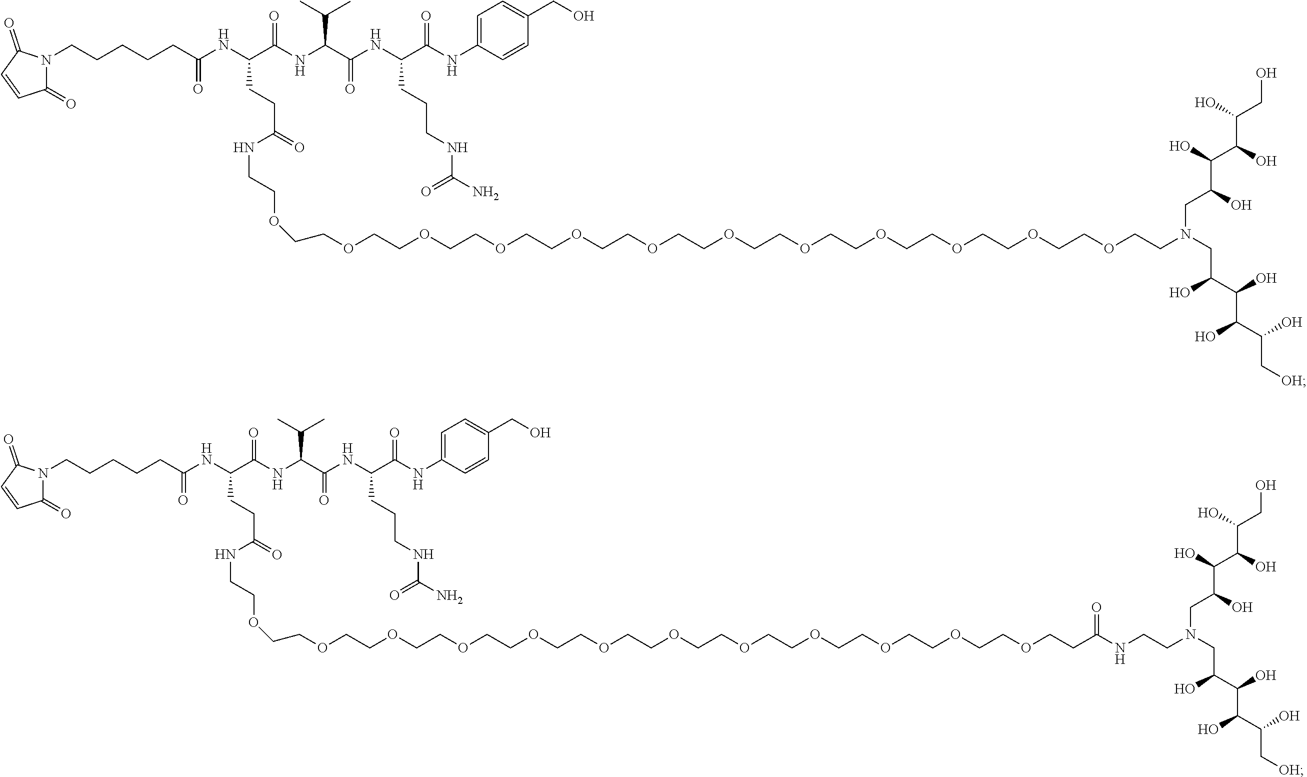

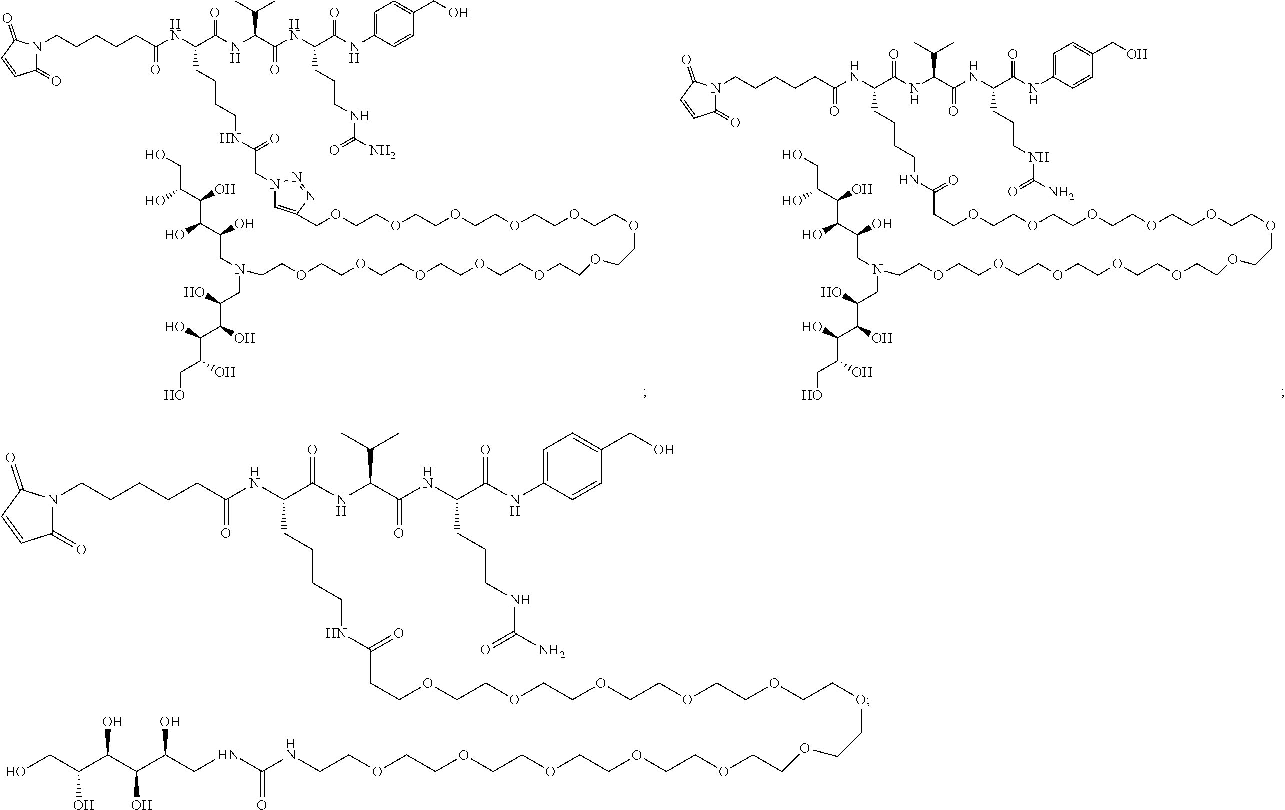

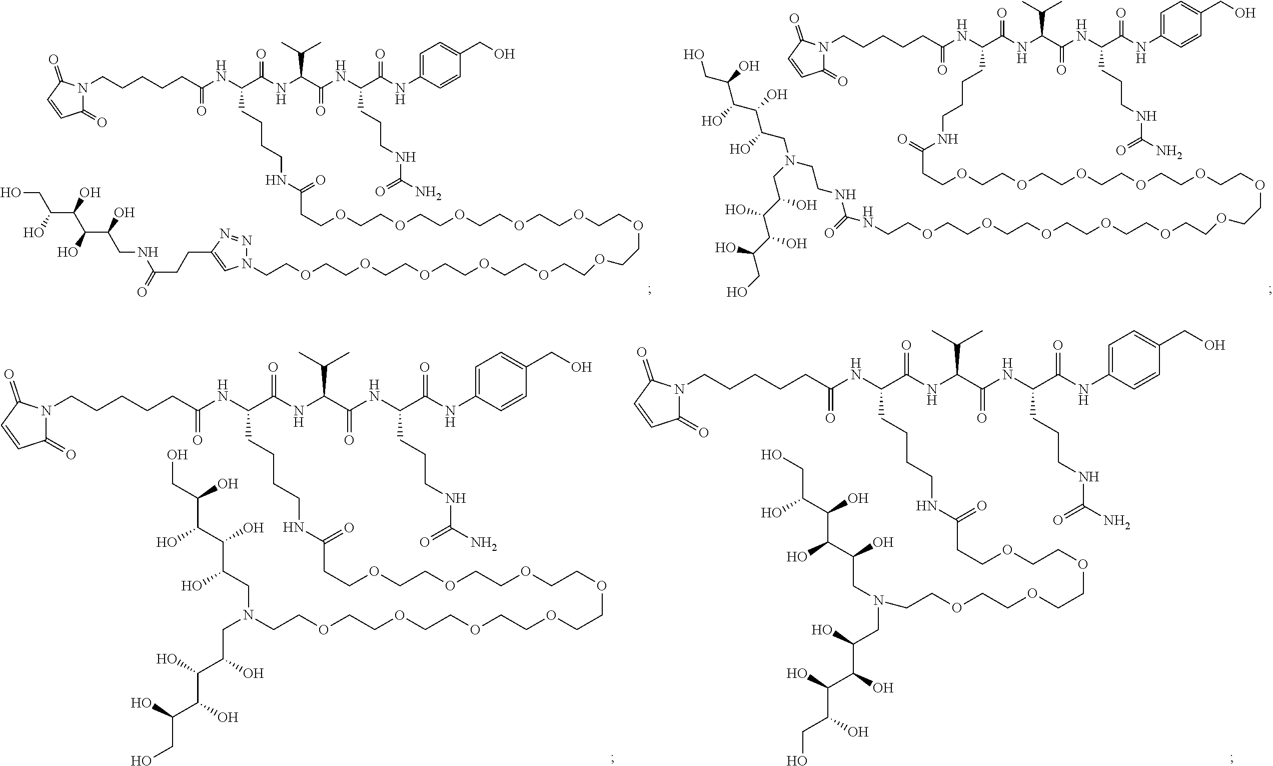

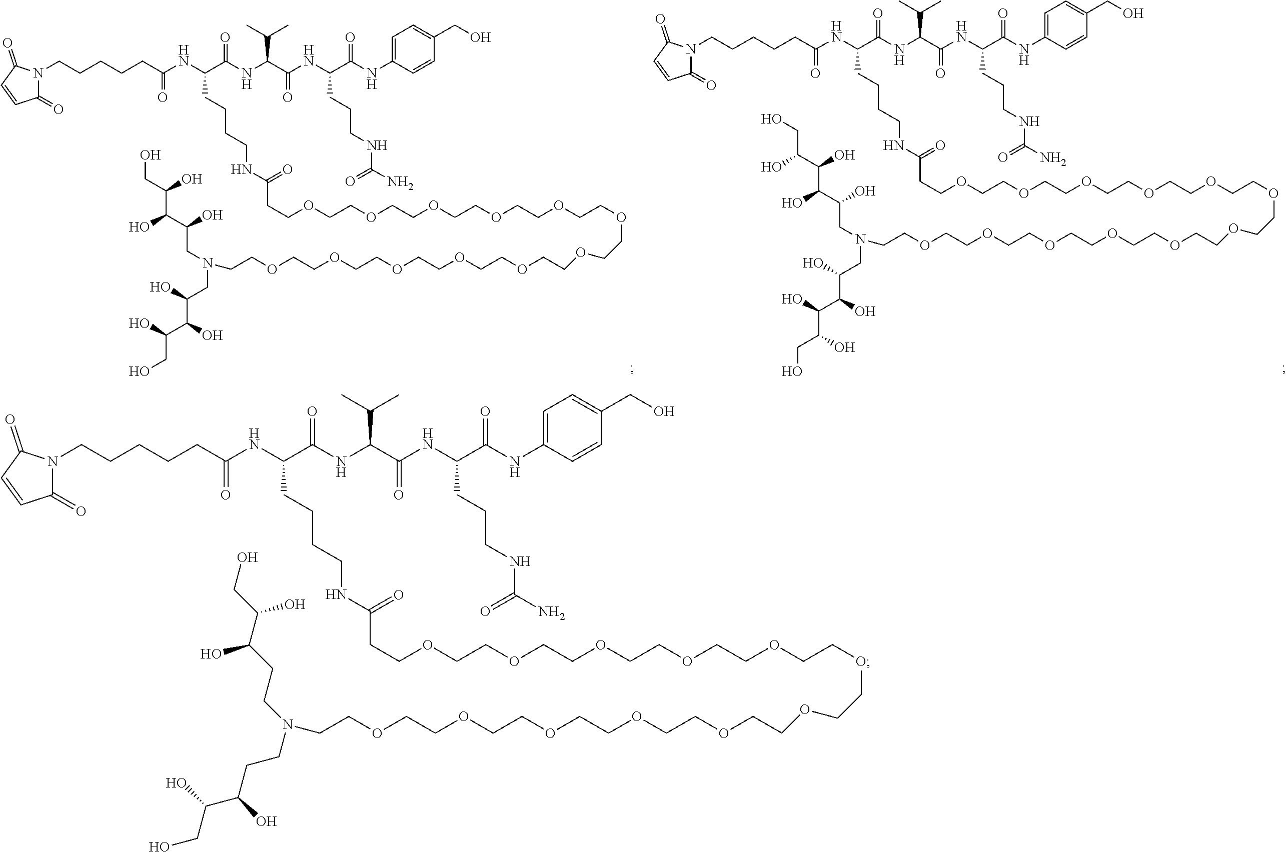

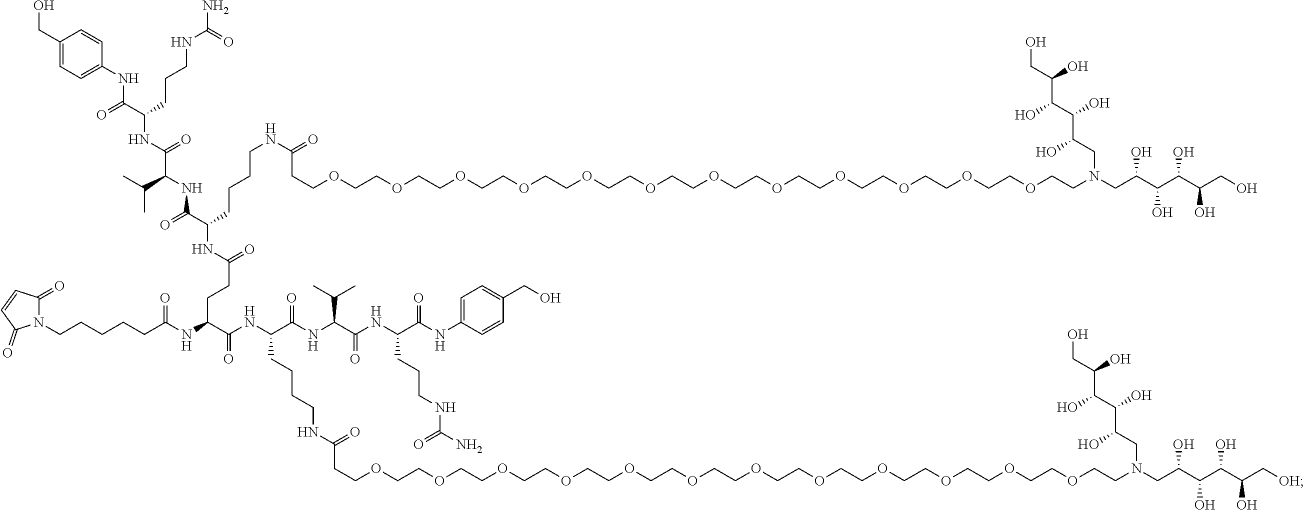

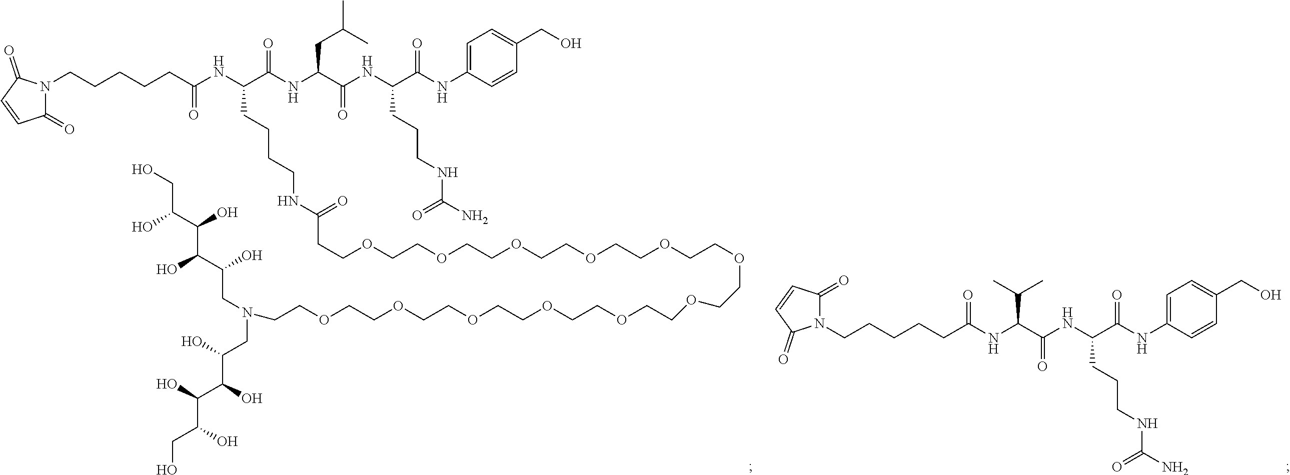

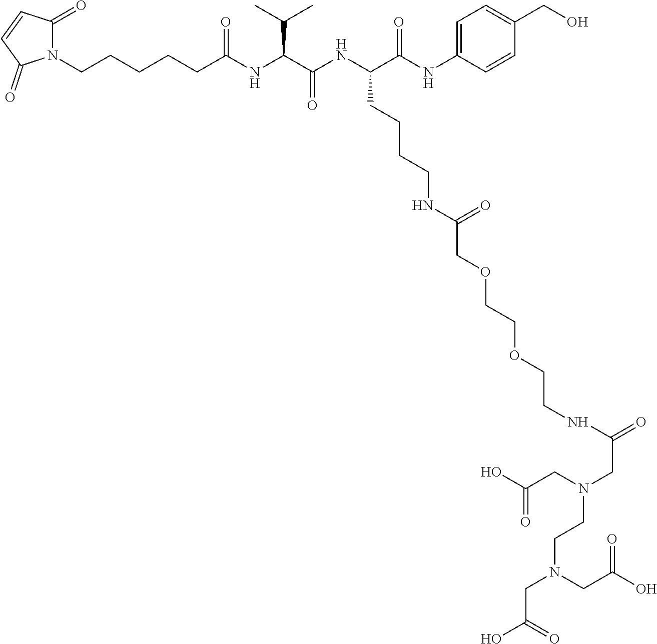

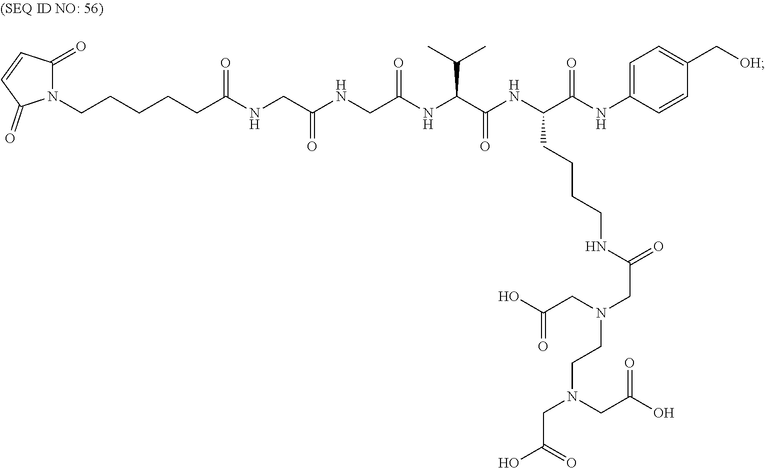

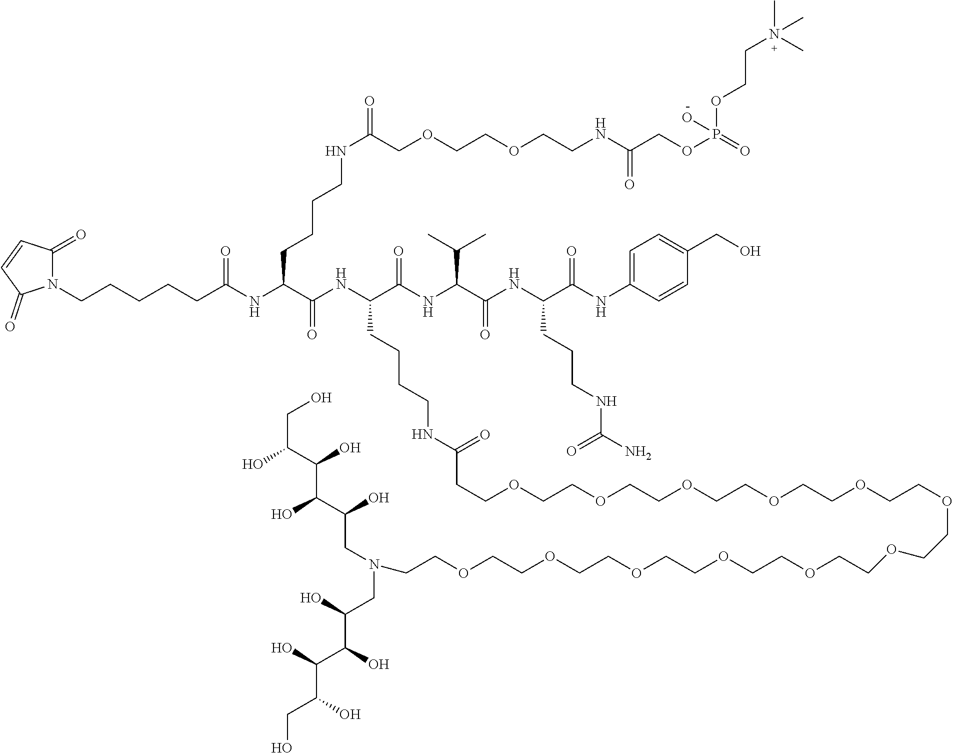

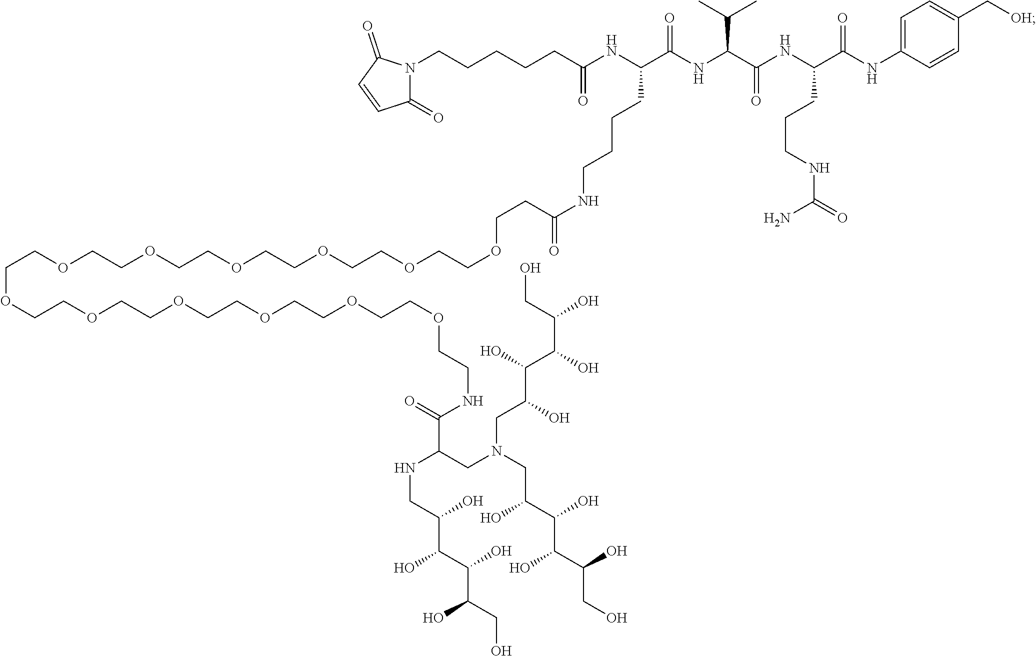

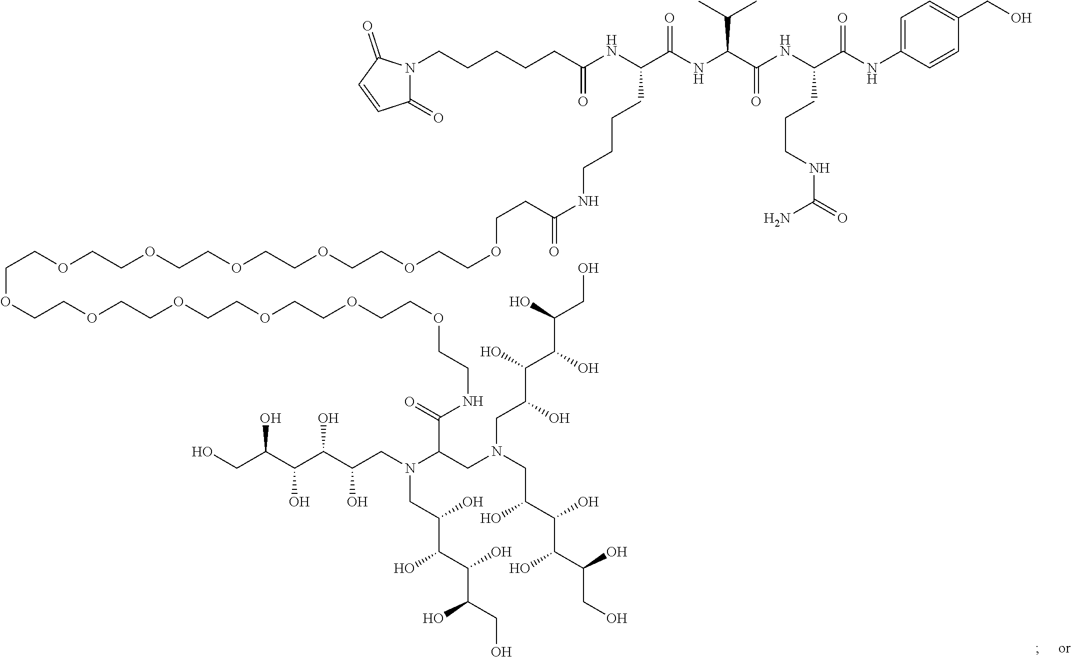

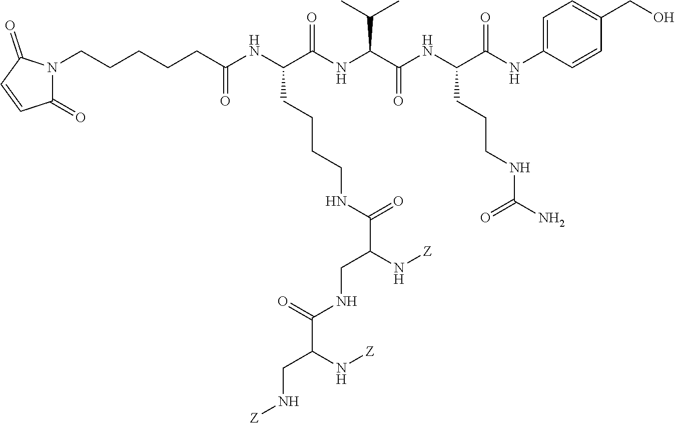

wherein each Z is attached at * and is individually selected from:

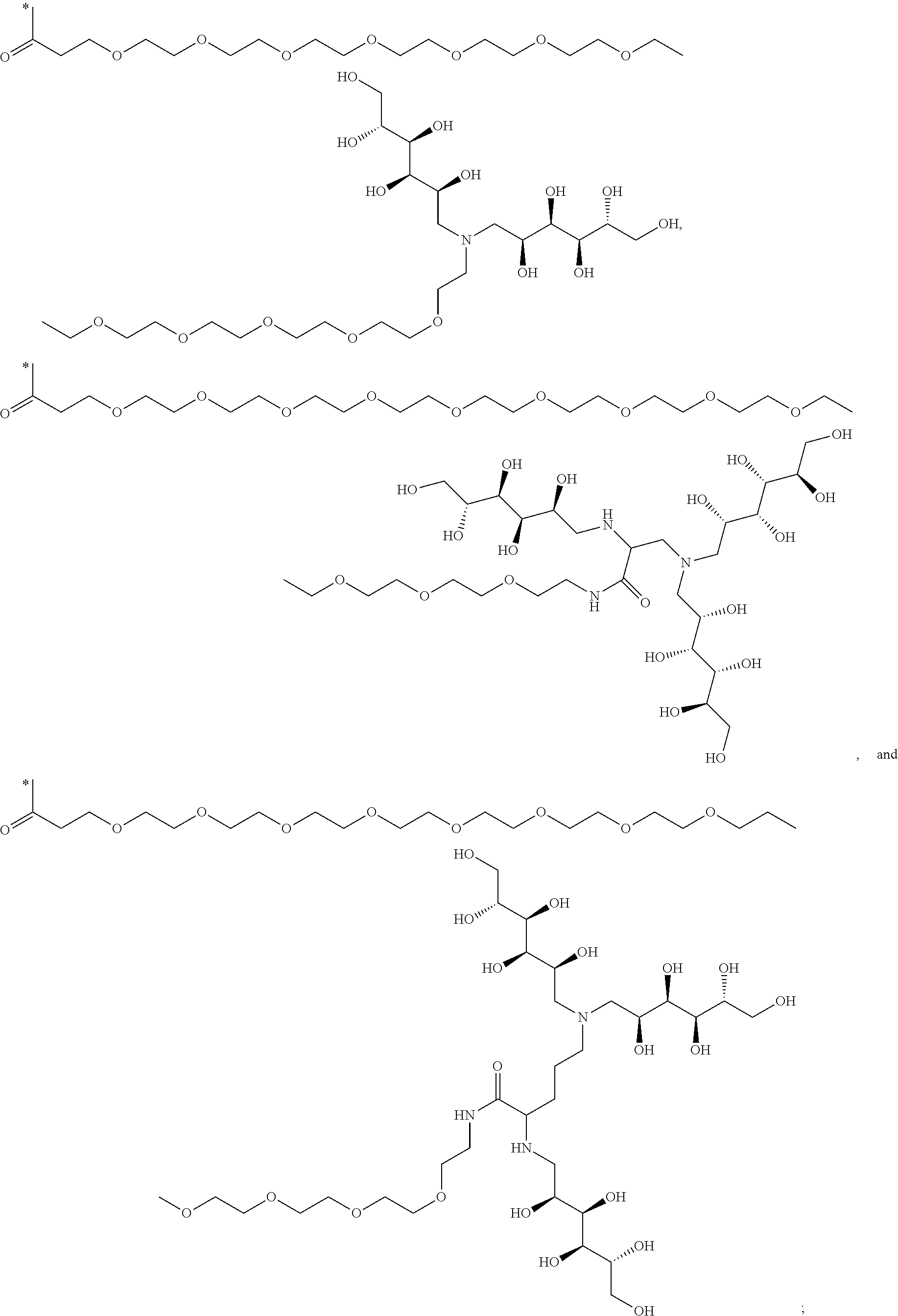

wherein a Drug unit is optionally attached to the terminal acid group or the benzyl alcohol, or wherein the wavy (≈) line indicates an attachment site for the Drug Unit.

In some embodiments, provided is a Linker, further comprising at least one Drug unit attached to Linker Subunit L2 to form a Drug-Linker. In some embodiments, provided is a Drug-Linker, wherein the Drug unit is selected from a cytotoxic agent, an immune modulatory agent, a nucleic acid, a growth inhibitory agent, a PROTAC, a toxin, a radioactive isotope and a chelating ligand. In some embodiments, provided is a Drug-Linker, wherein the Drug unit is a cytotoxic agent. In some embodiments, provided is a Drug-Linker, wherein the cytotoxic agent is selected from the group consisting of an auristatin, a maytansinoid, a camptothecin, a duocarmycin, and a calicheamicin. In some embodiments, provided is a Drug-Linker, wherein the cytotoxic agent is an auristatin. In some embodiments, provided is a Drug-Linker, wherein the cytotoxic agent is MMAE or MMAF. In some embodiments, provided is a Drug-Linker, wherein the cytotoxic agent is a camptothecin. In some embodiments, provided is a Drug-Linker, wherein the cytotoxic agent is exatecan or SN-38. In some embodiments, provided is a Drug-Linker, wherein the cytotoxic agent is exatecan. In some embodiments, provided is a Drug-Linker, wherein the cytotoxic agent is a calicheamicin. In some embodiments, provided is a Drug-Linker, wherein the cytotoxic agent is a maytansinoid. In some embodiments, provided is a Drug-Linker, wherein the maytansinoid is maytansine, maytansinol or ansamatocin-2.

In some embodiments, provided is a Drug-Linker, wherein the cytotoxic agent is a calicheamicin. In some embodiments, provided is a Drug-Linker, wherein the cytotoxic agent is a maytansinoid. In some embodiments, provided is a Drug-Linker, wherein the maytansinoid is maytansine, maytansinol or ansamatocin-2.

In some embodiments, provided is a Drug-Linker, wherein the Drug unit is an immune modulatory agent. In some embodiments, provided is a Drug-Linker, wherein the immune modulatory agent is selected from a TRL7 agonist, a TLR8 agonist, a STING agonist, or a RIG-1 agonist. In some embodiments, provided is a Drug-Linker, wherein the immune modulatory agent is an TLR7 agonist. In some embodiments, provided is a Drug-Linker, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3. In some embodiments, provided is a Drug-Linker, wherein the immune modulatory agent is a TLR8 agonist. In some embodiments, provided is a Drug-Linker, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, provided is a Drug-Linker, wherein the immune modulatory agent is a STING agonist. In some embodiments, provided is a Drug-Linker, wherein the immune modulatory agent is a RIG-1 agonist. In some embodiments, provided is a Drug-Linker, wherein the RIG-1 agonist is selected from KIN1148, SB-9200, KIN700, KIN600, KIN500, KIN100, KIN101, KIN400 and KIN2000.

In some embodiments, provided is a Drug-Linker, wherein the Drug unit is a chelating ligand. In some embodiments, provided is a Drug-Linker, wherein the chelating ligand is selected from platinum (Pt), ruthenium (Ru), rhodium (Rh), gold (Au), silver (Ag), copper (Cu), molybdenum (Mo), titanium (Ti), or iridum (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

In some embodiments, provided is a Drug-Linker, having the following structure:

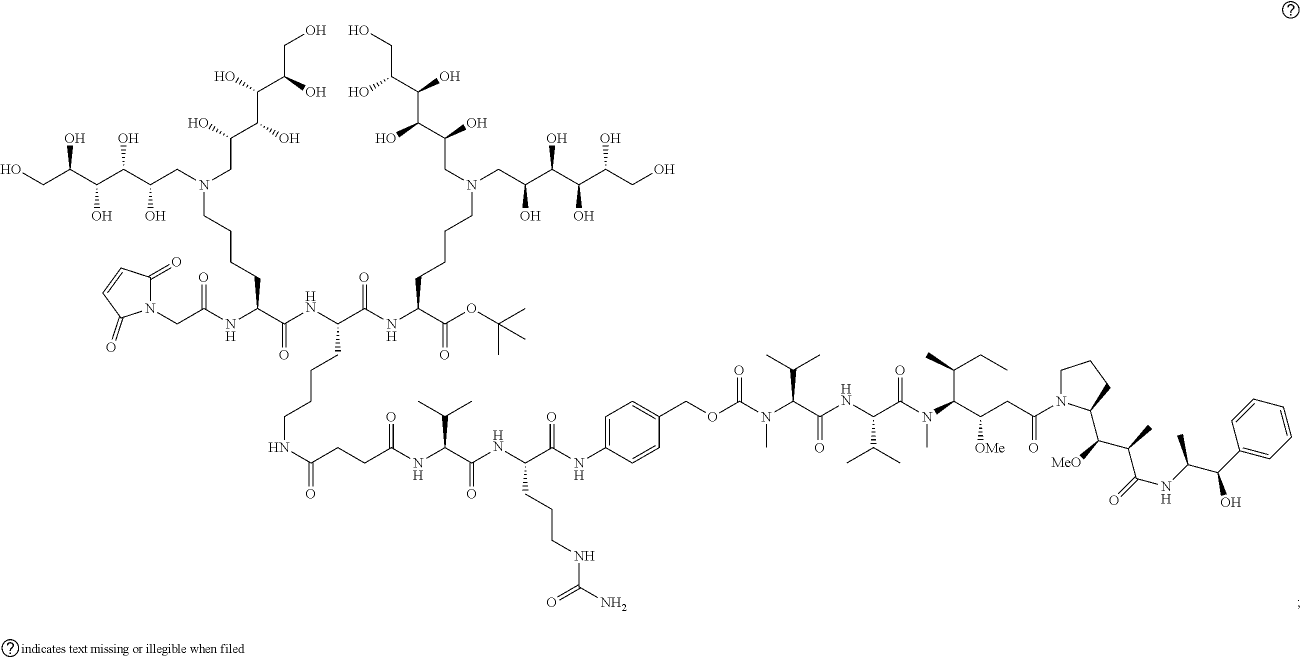

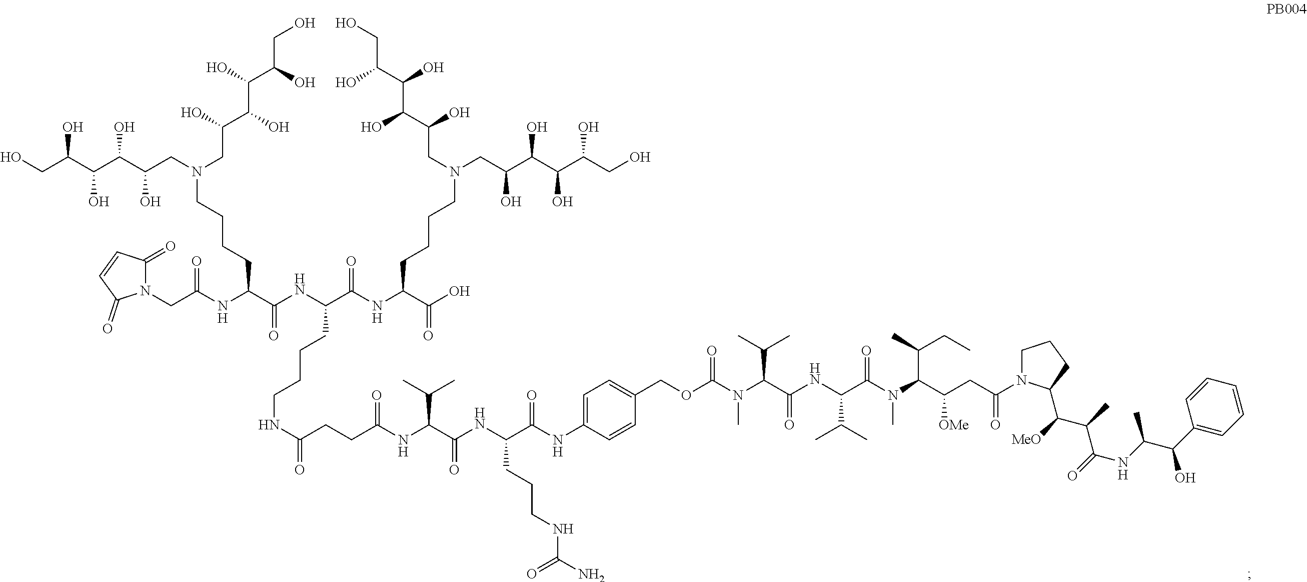

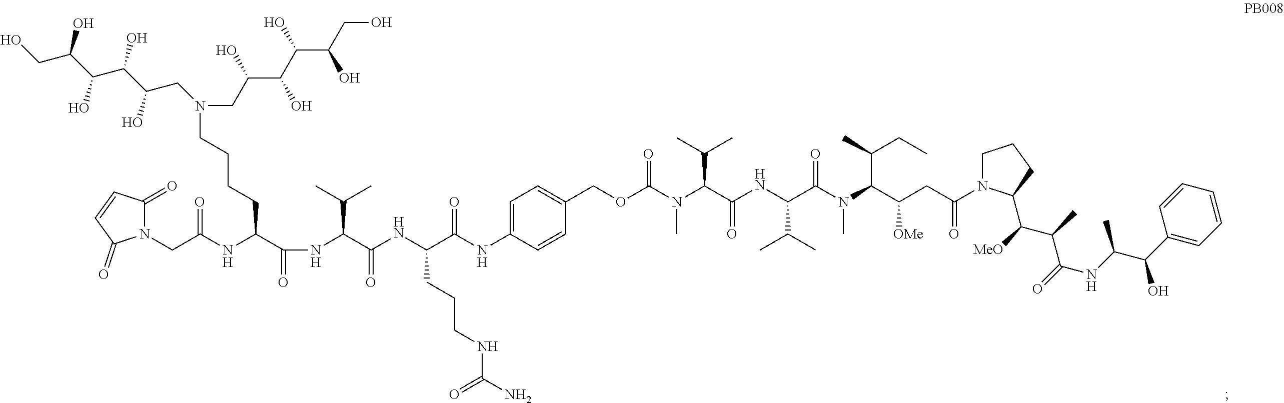

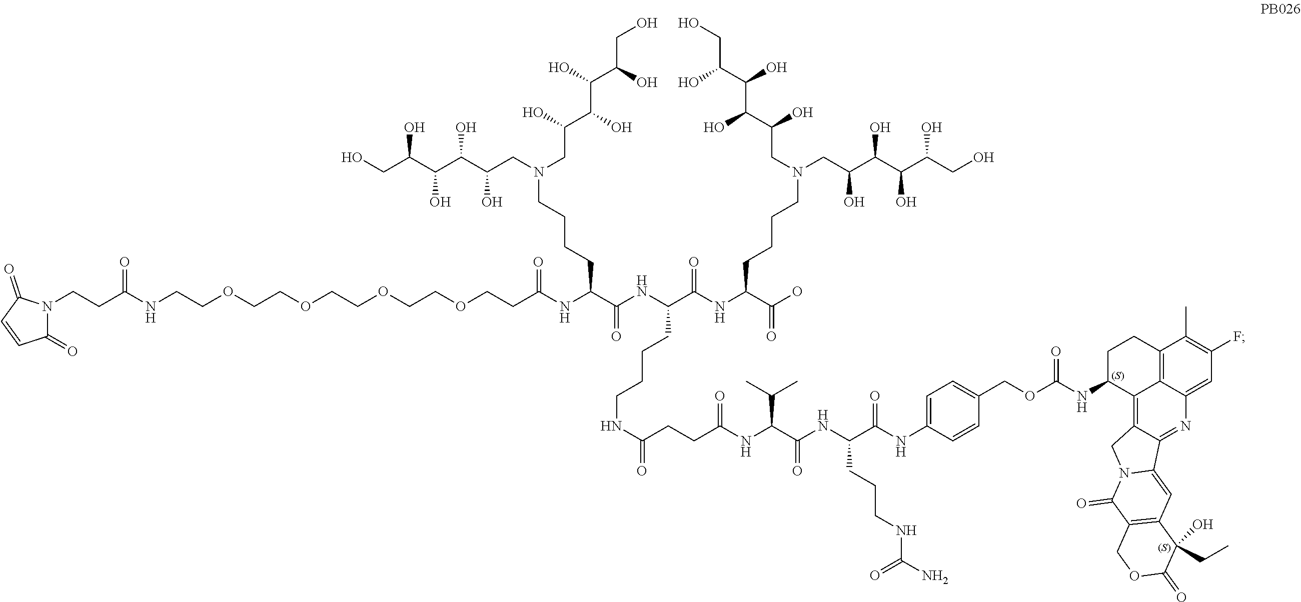

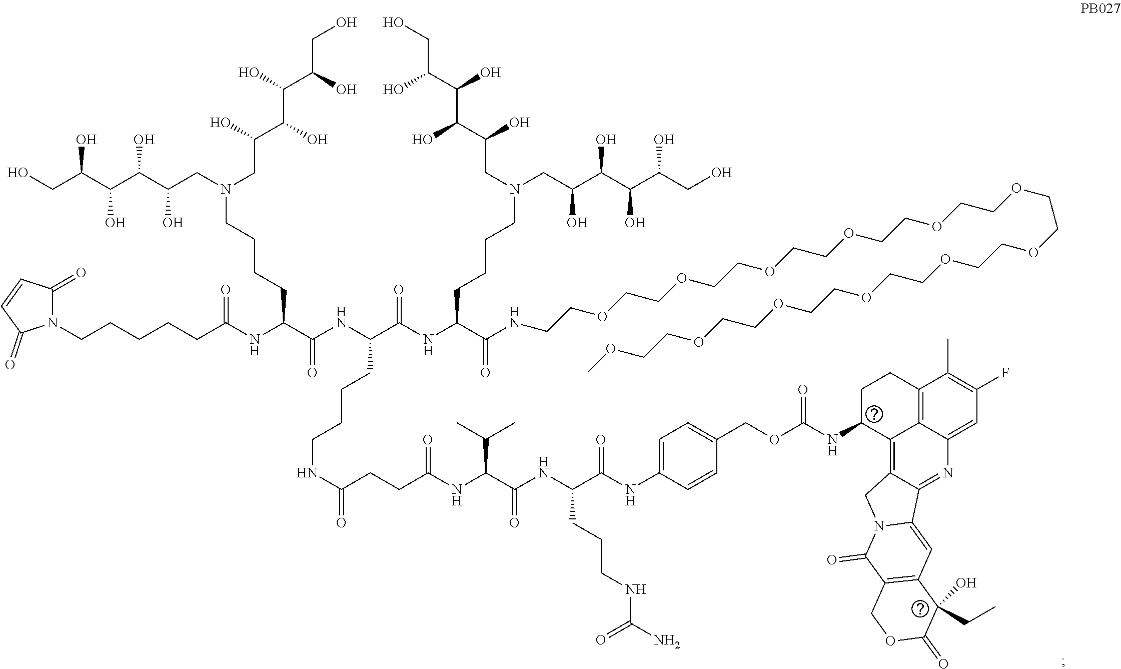

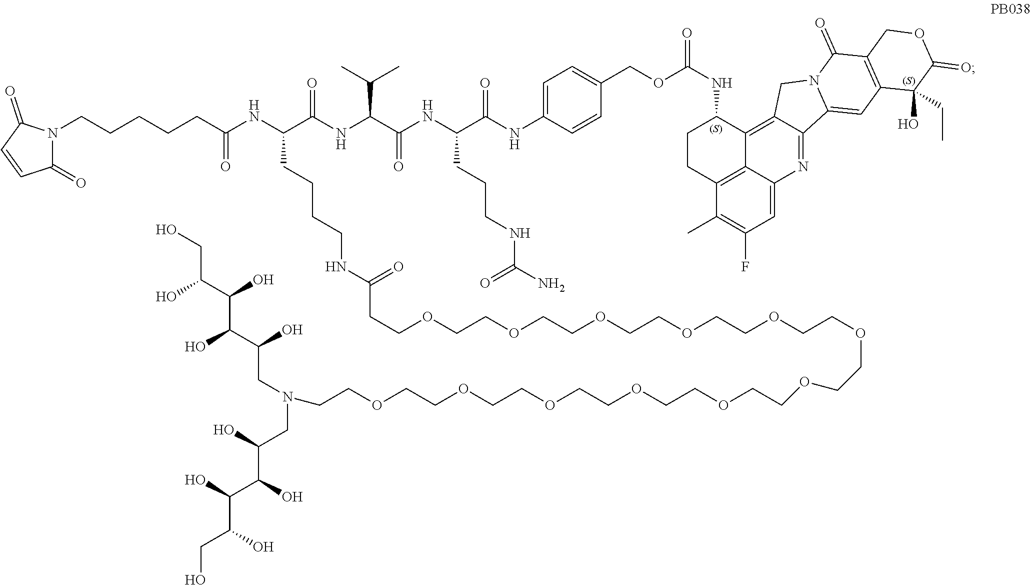

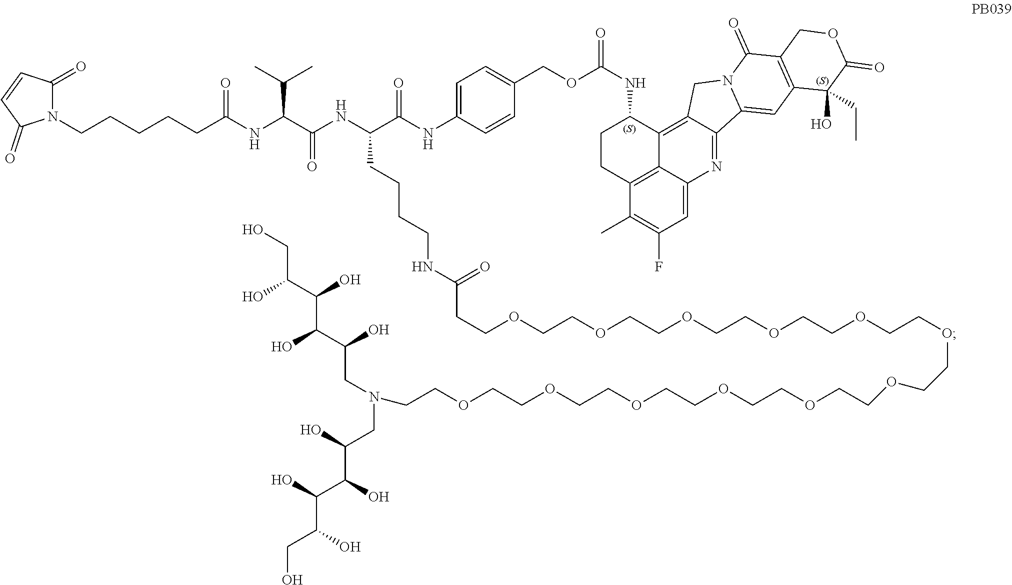

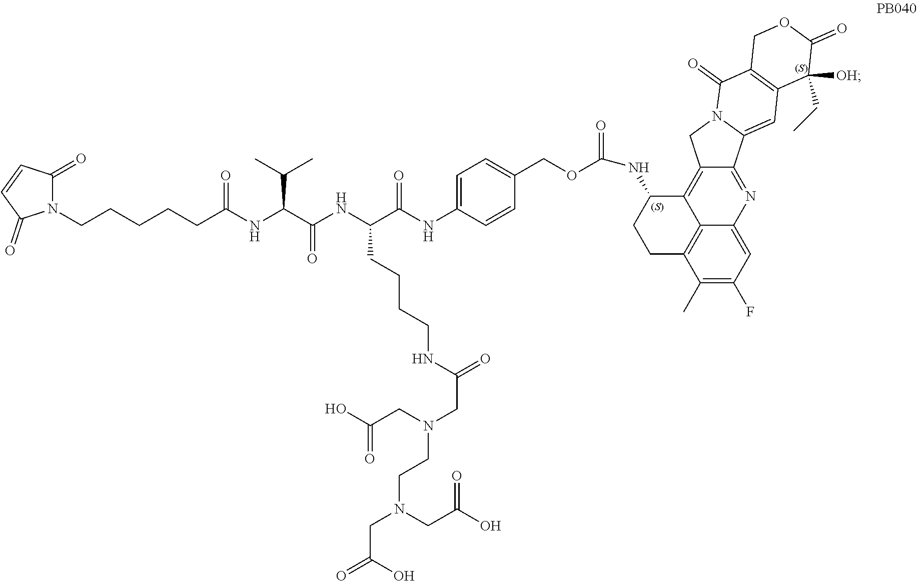

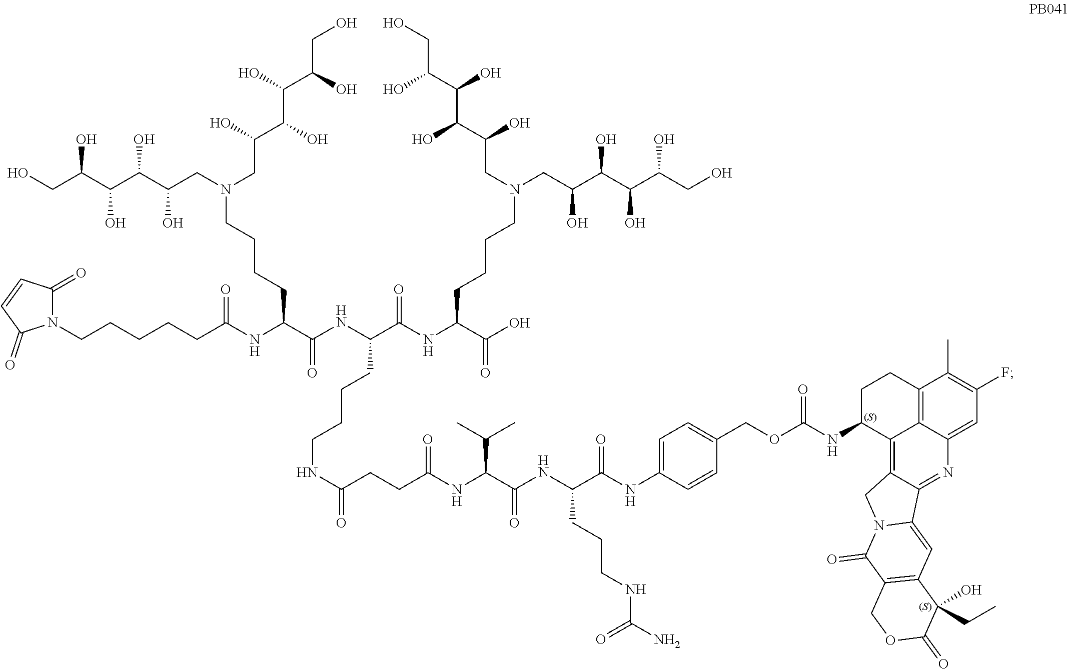

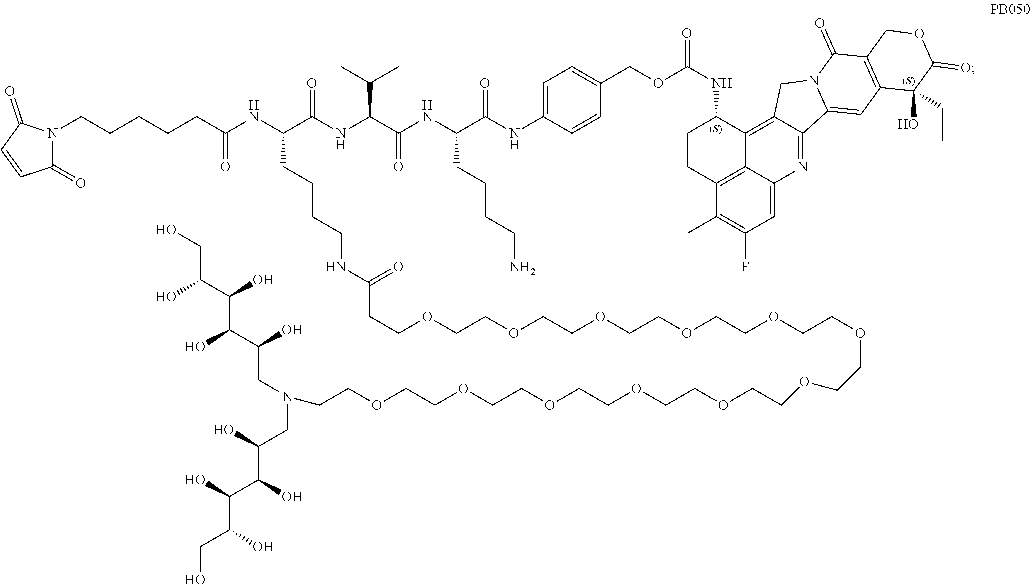

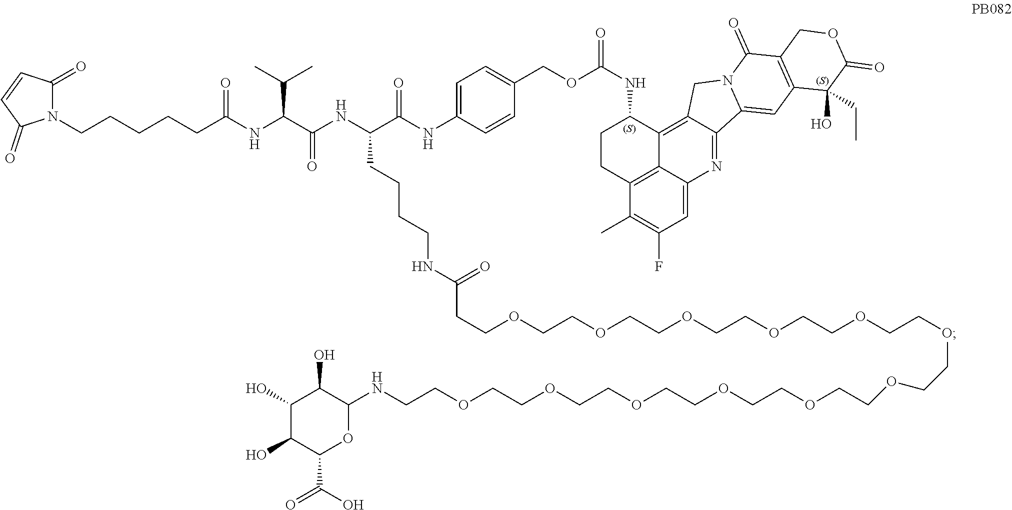

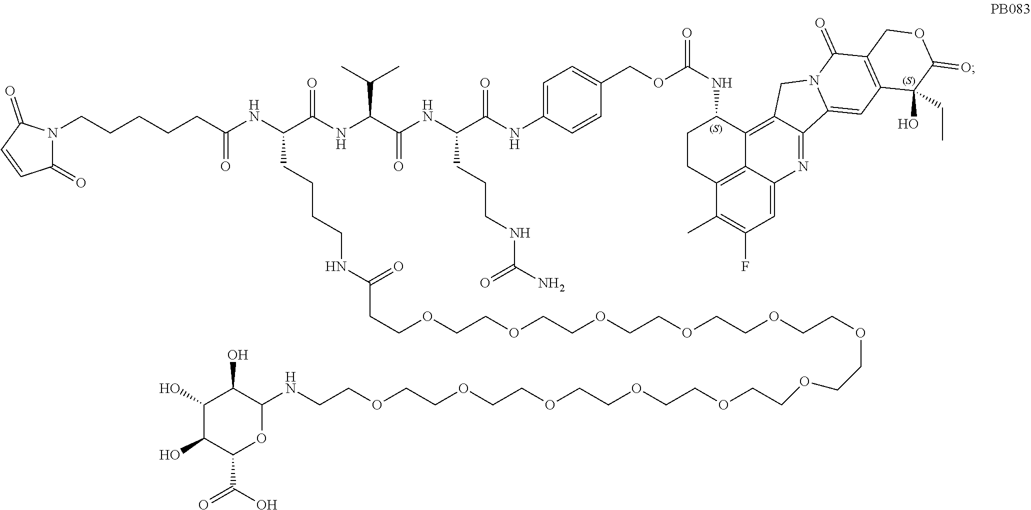

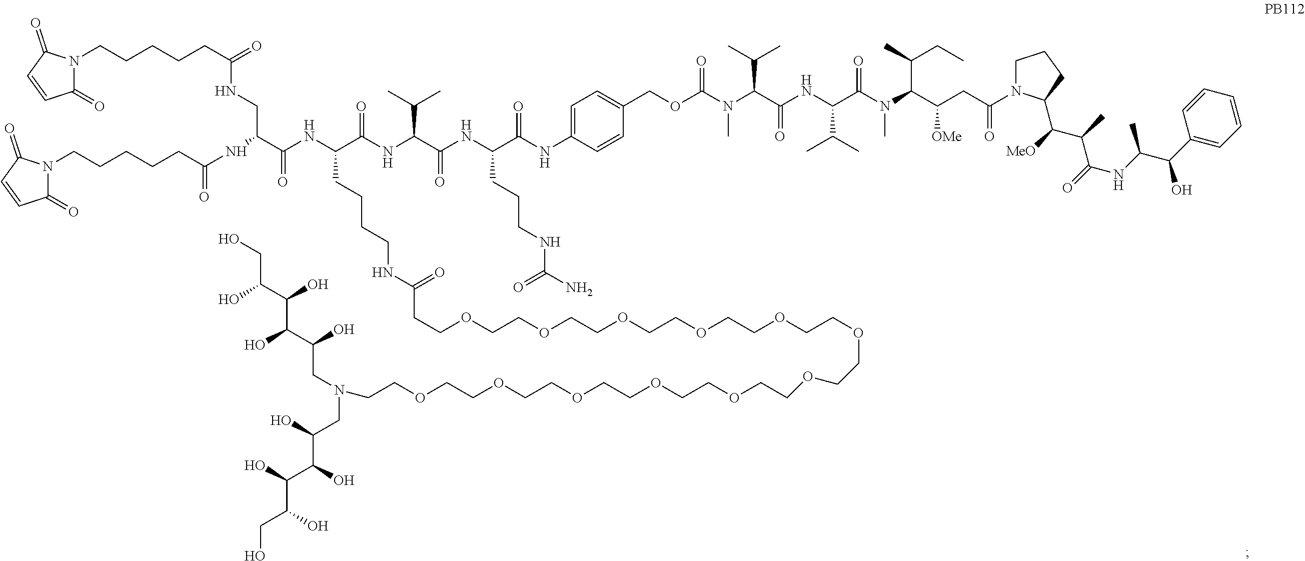

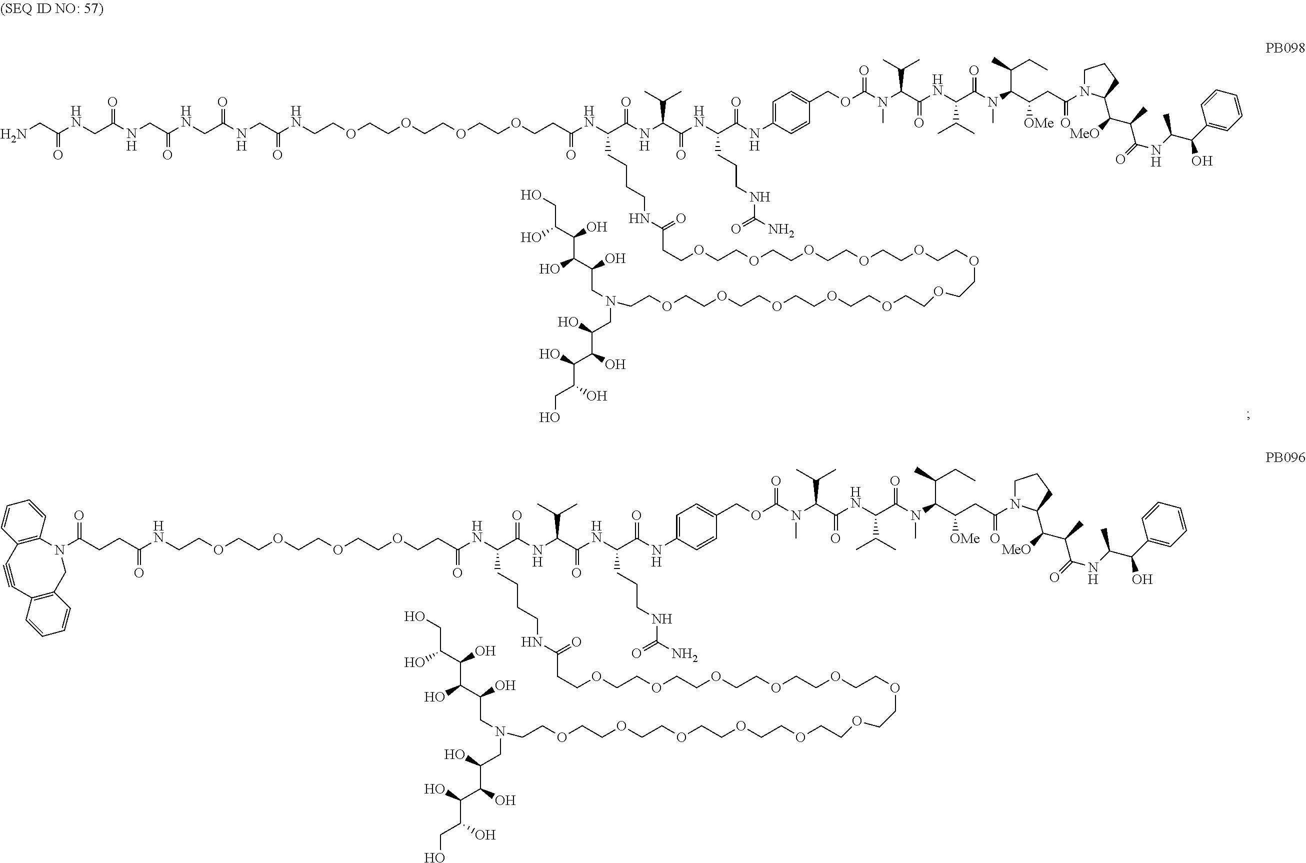

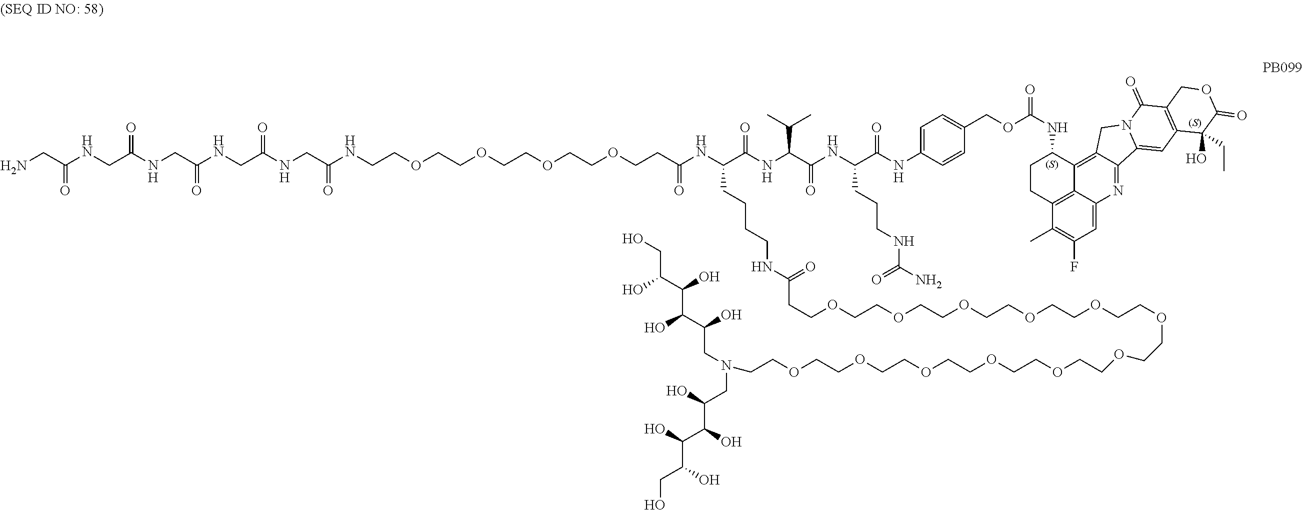

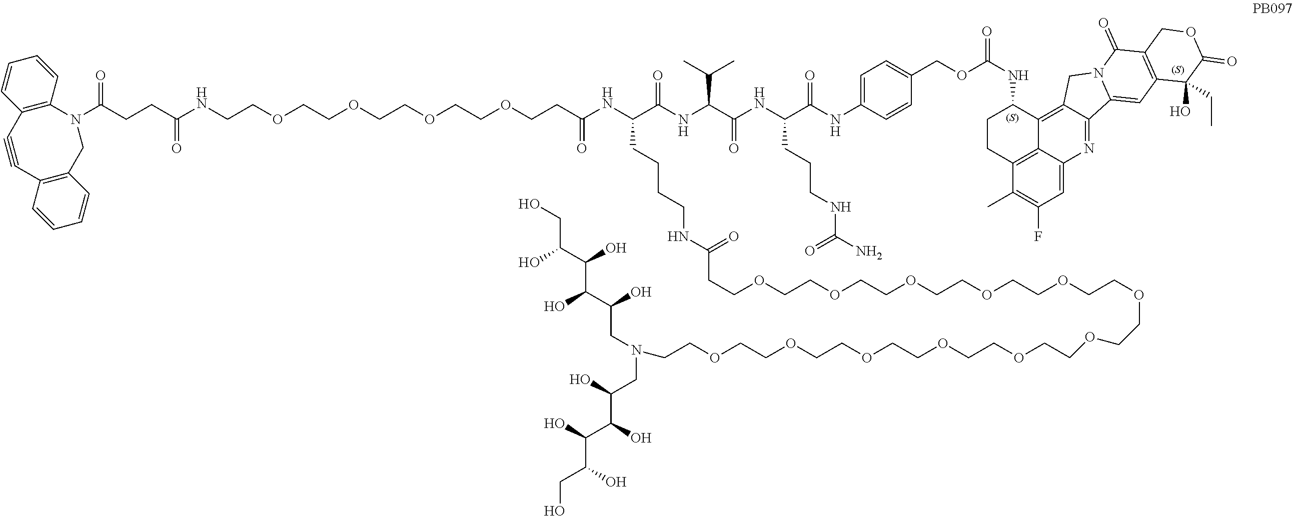

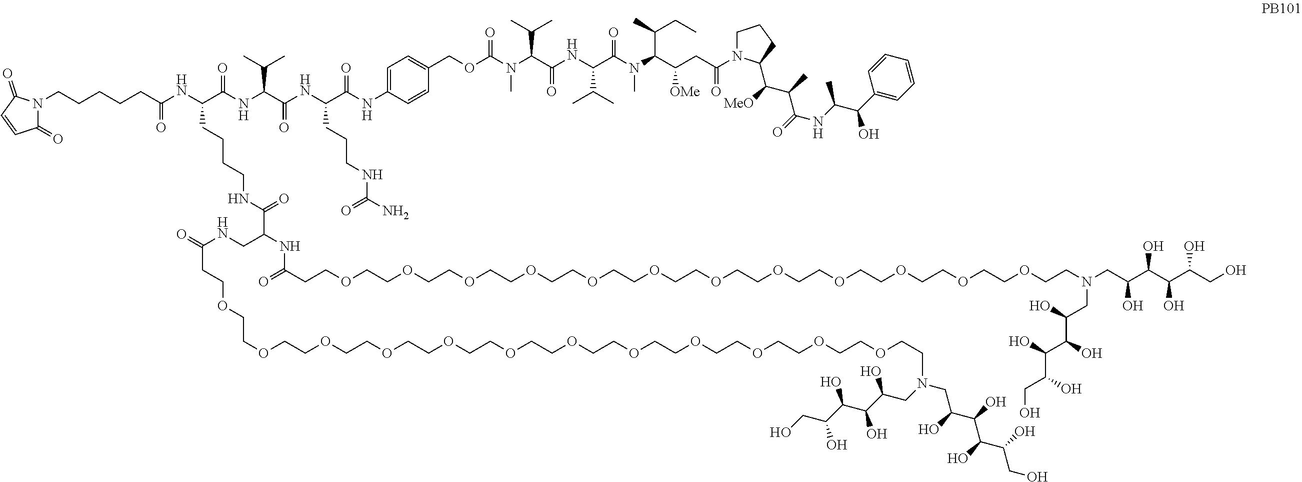

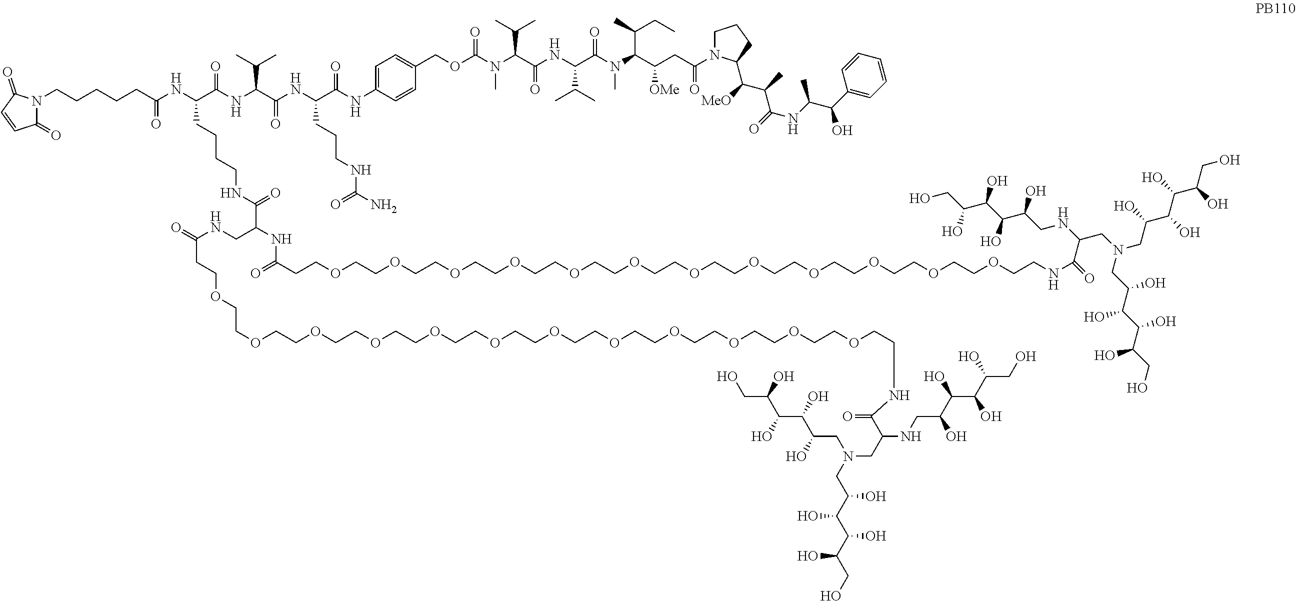

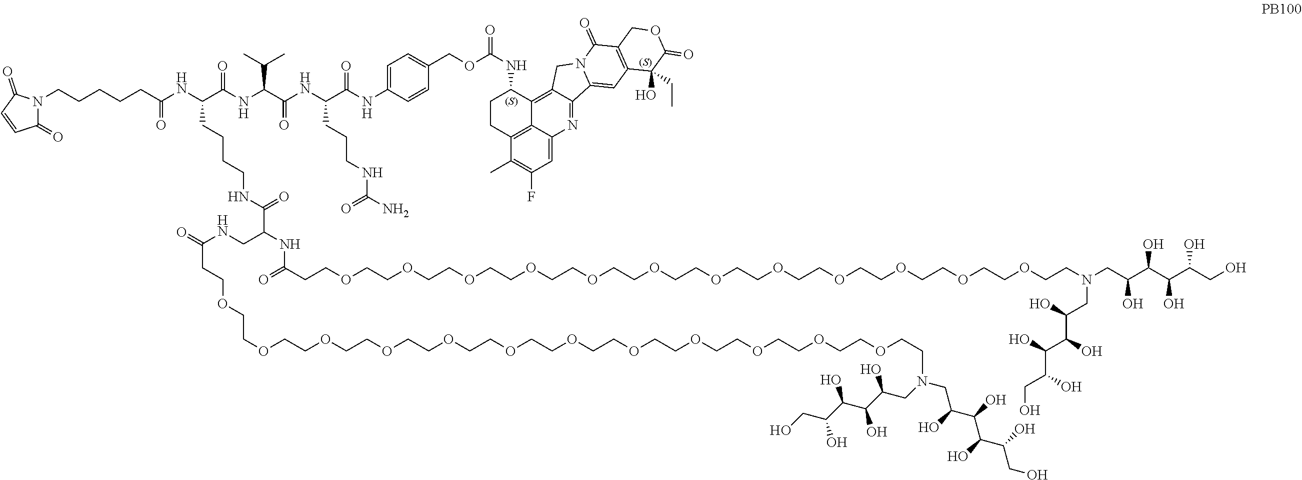

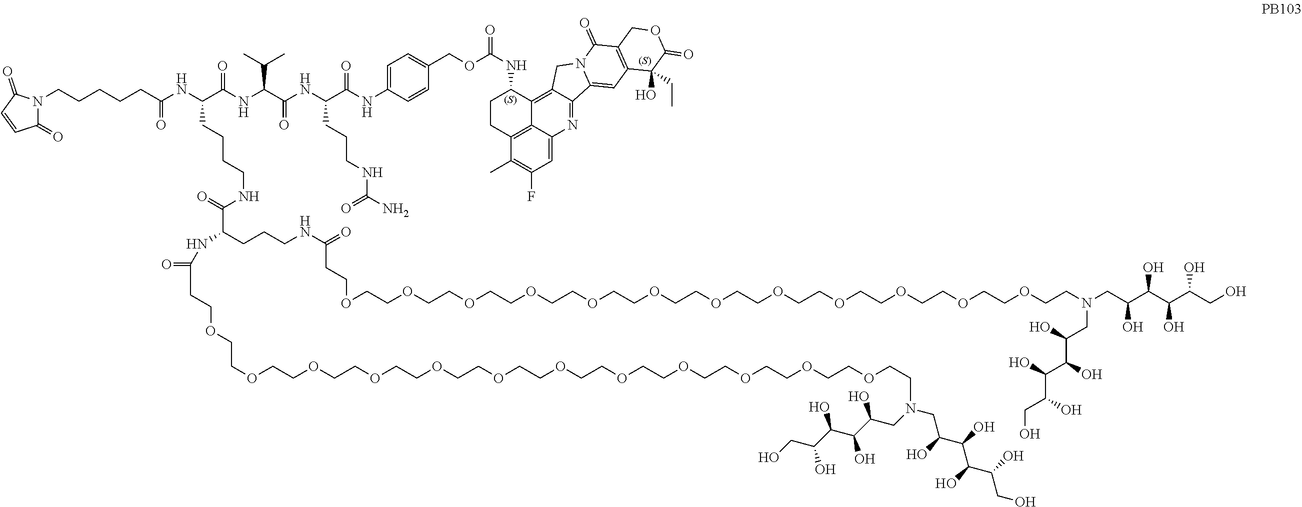

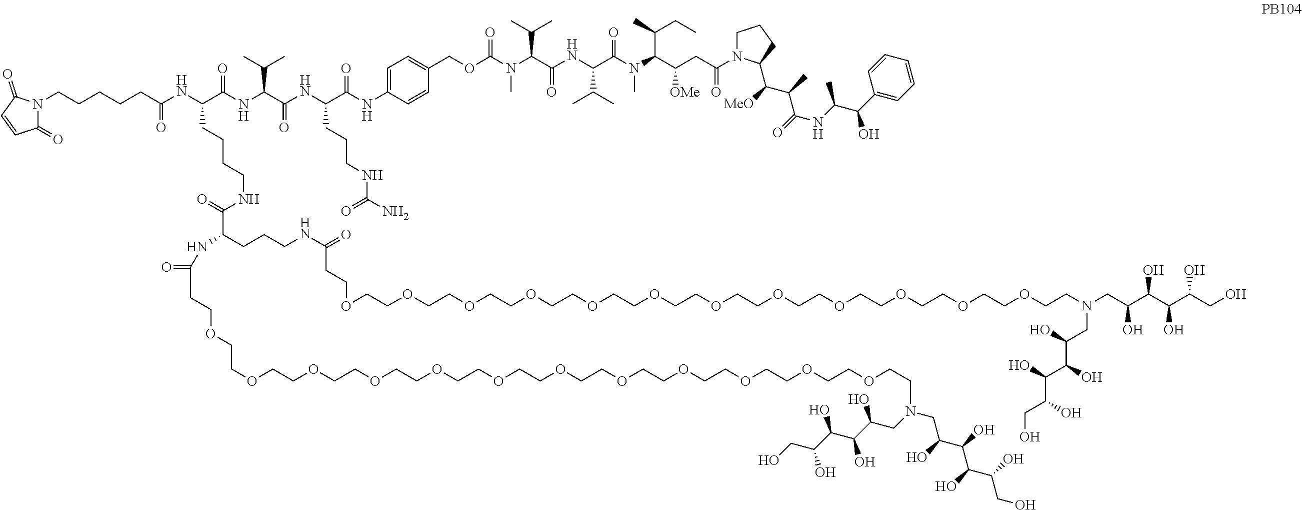

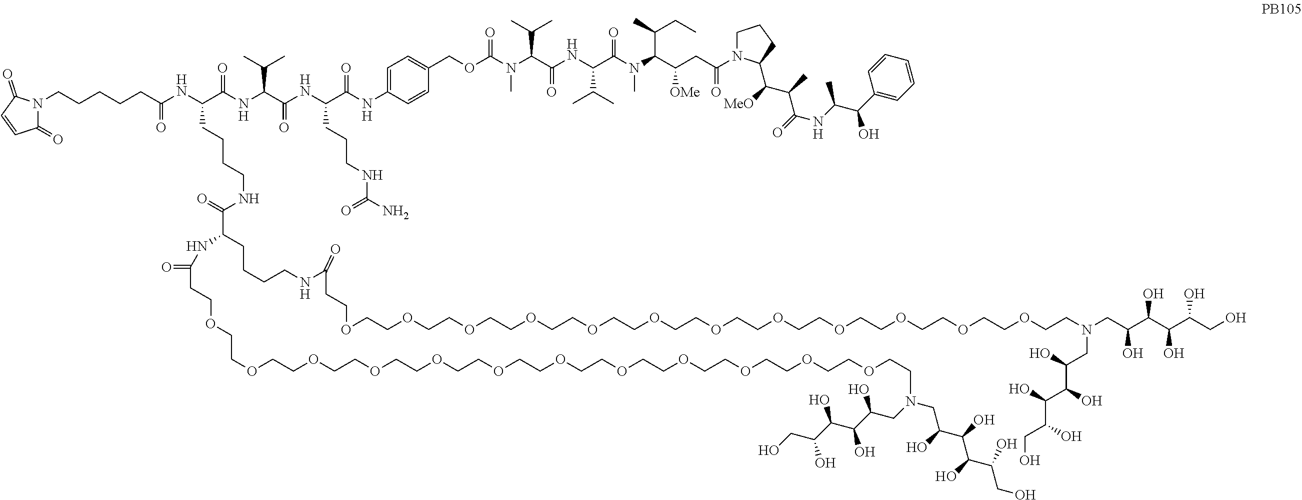

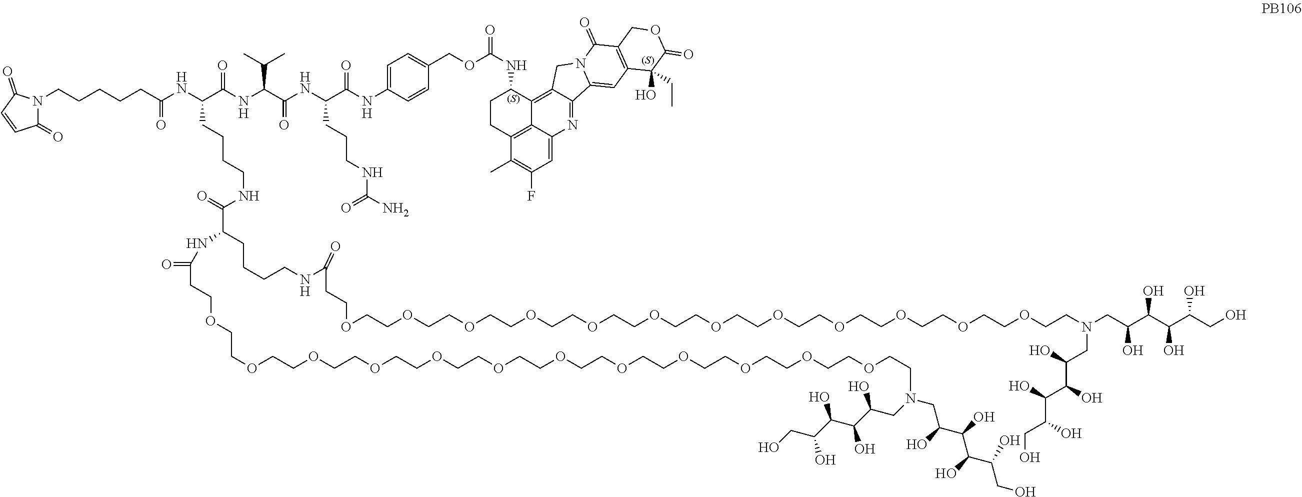

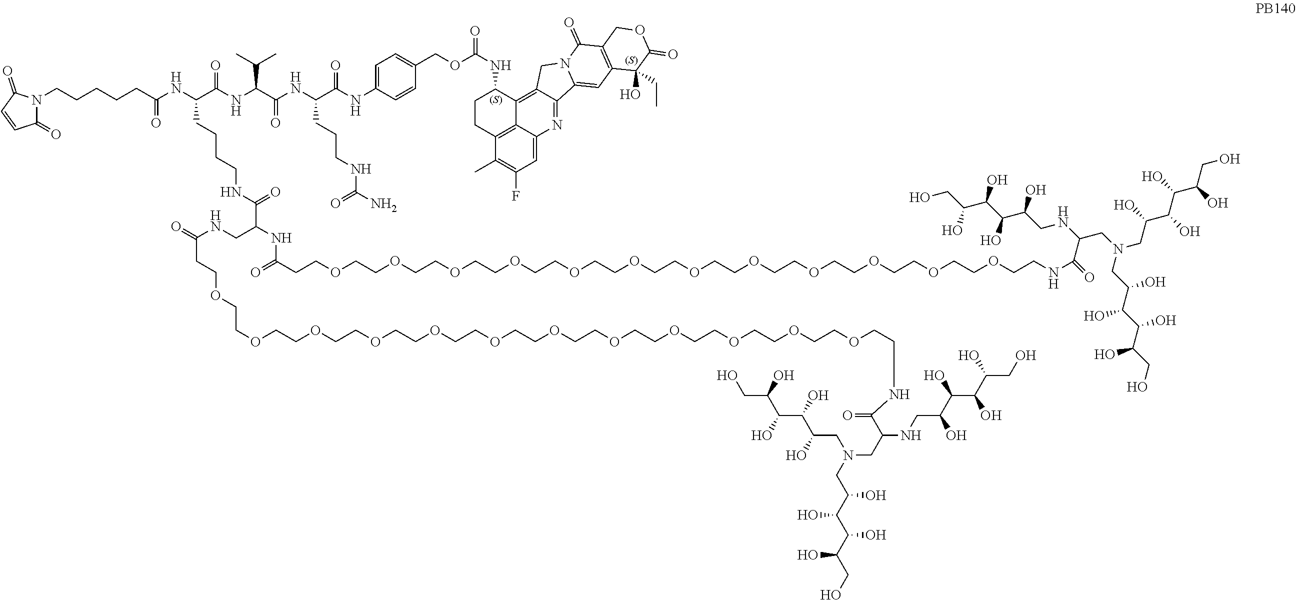

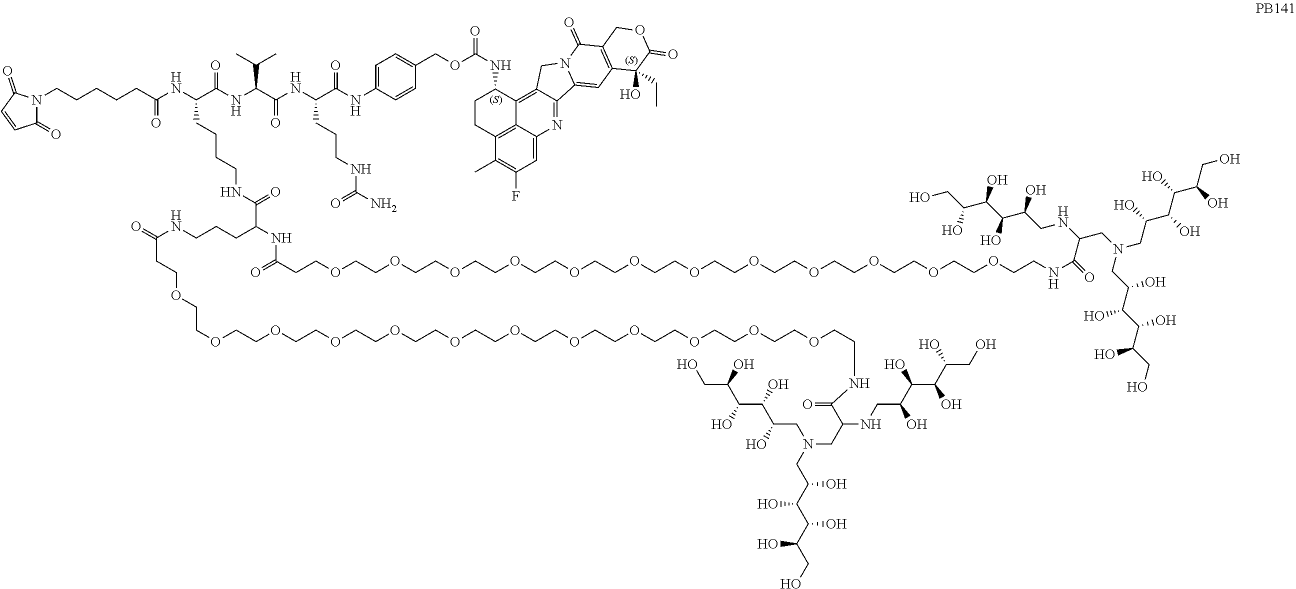

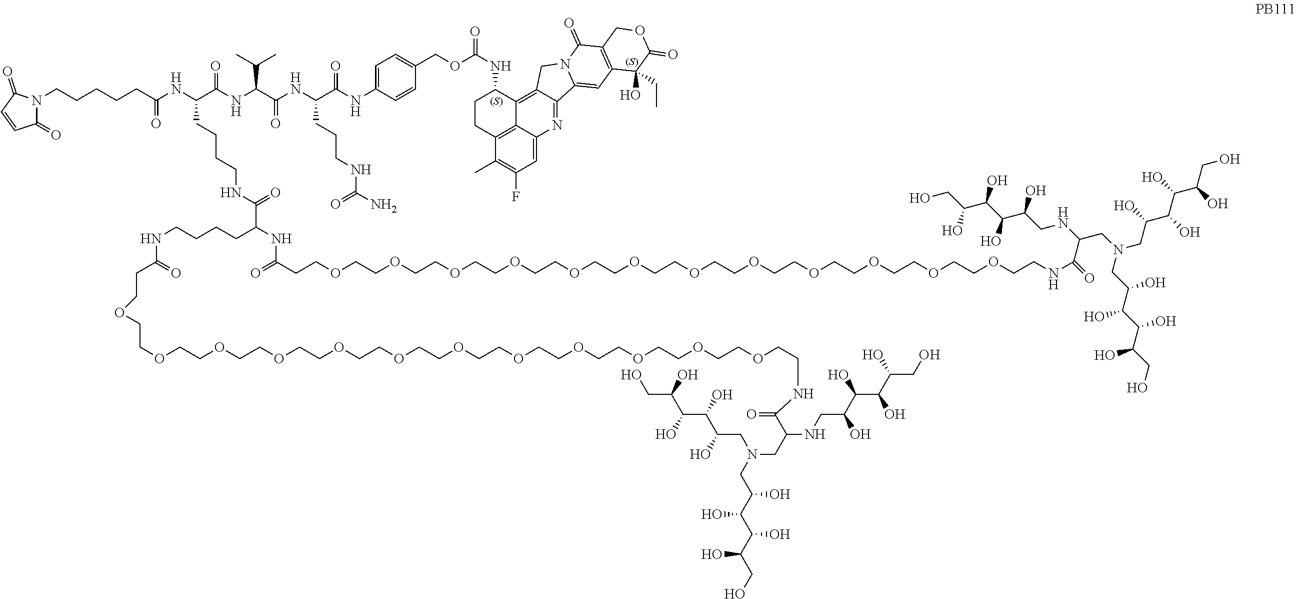

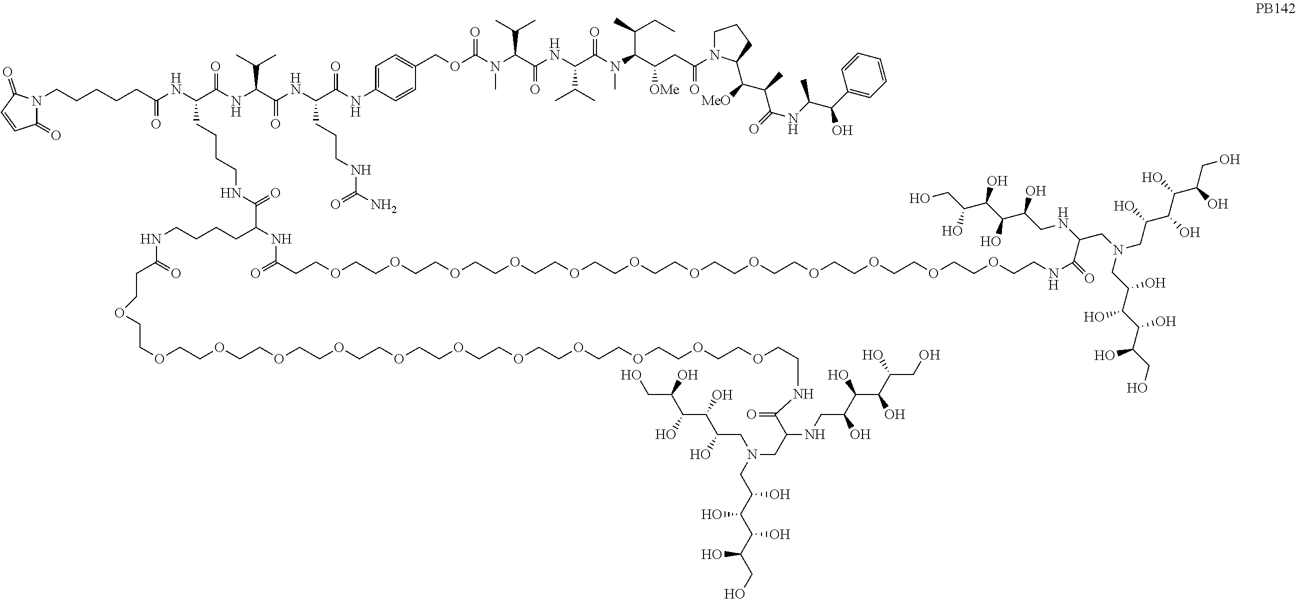

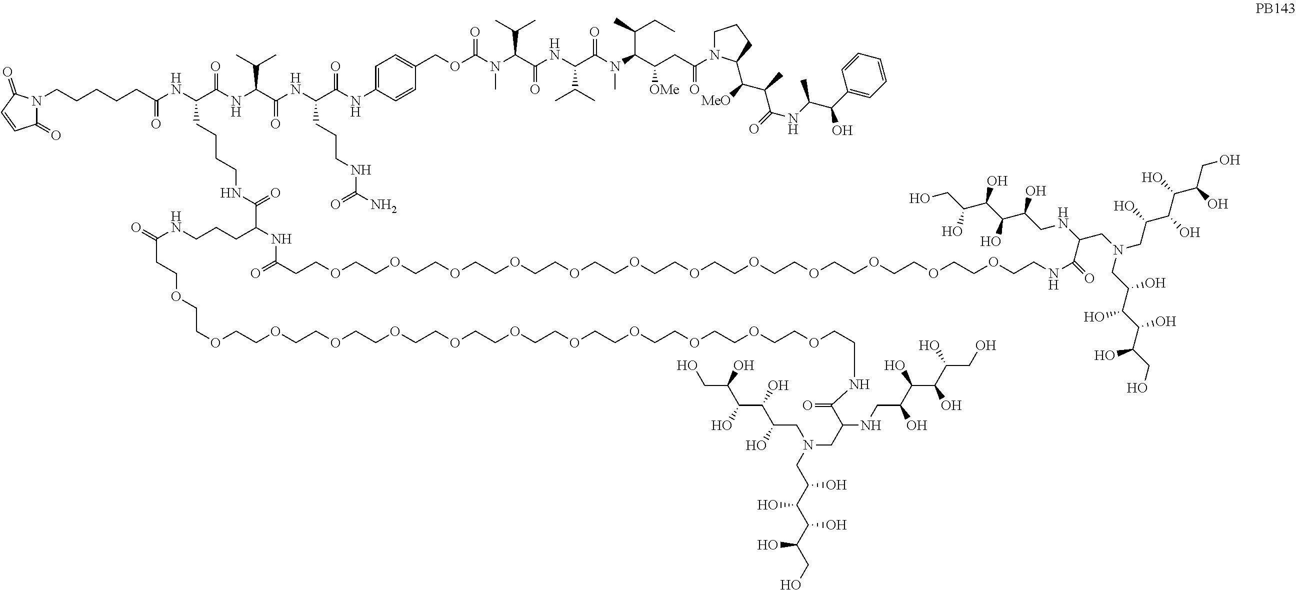

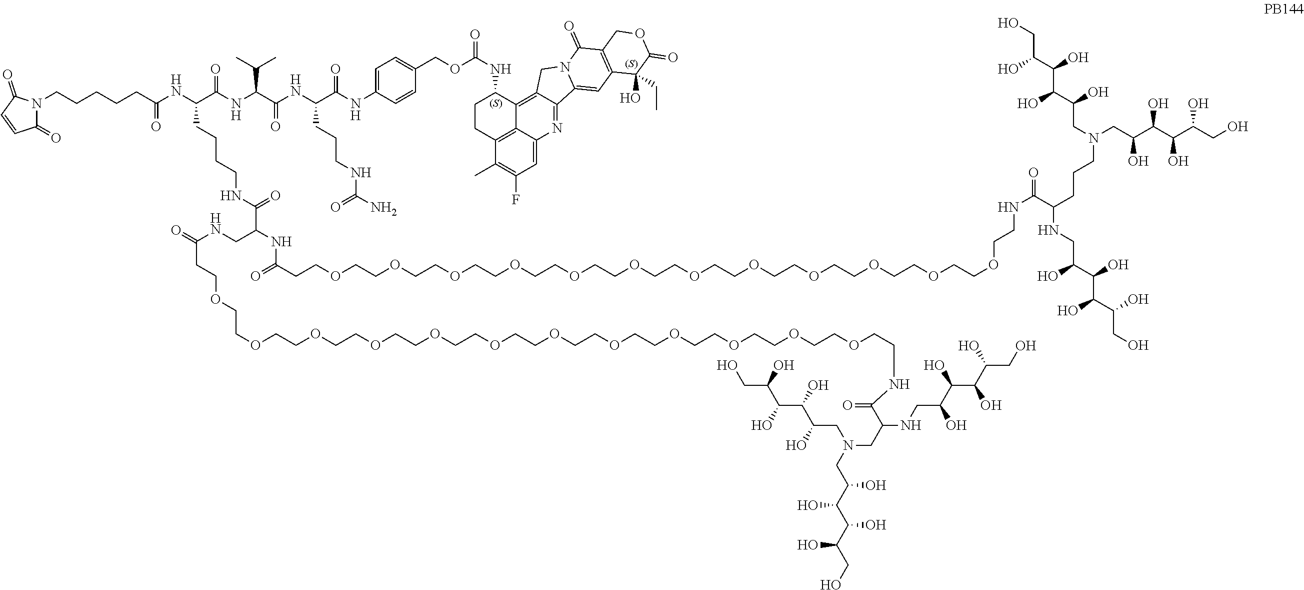

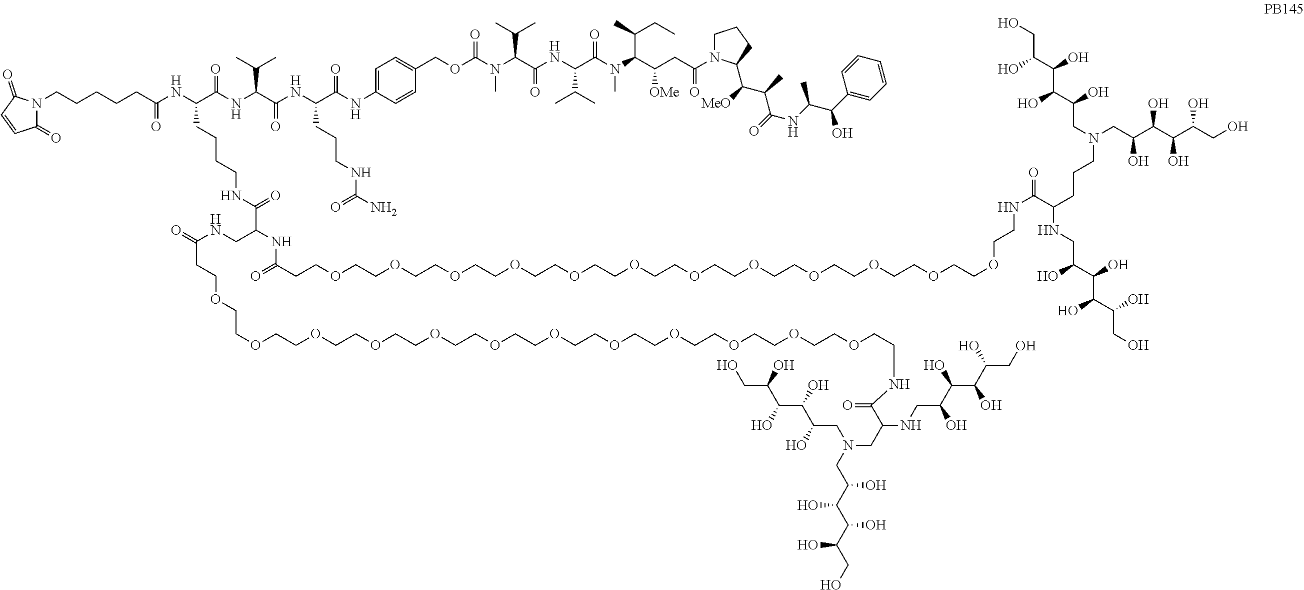

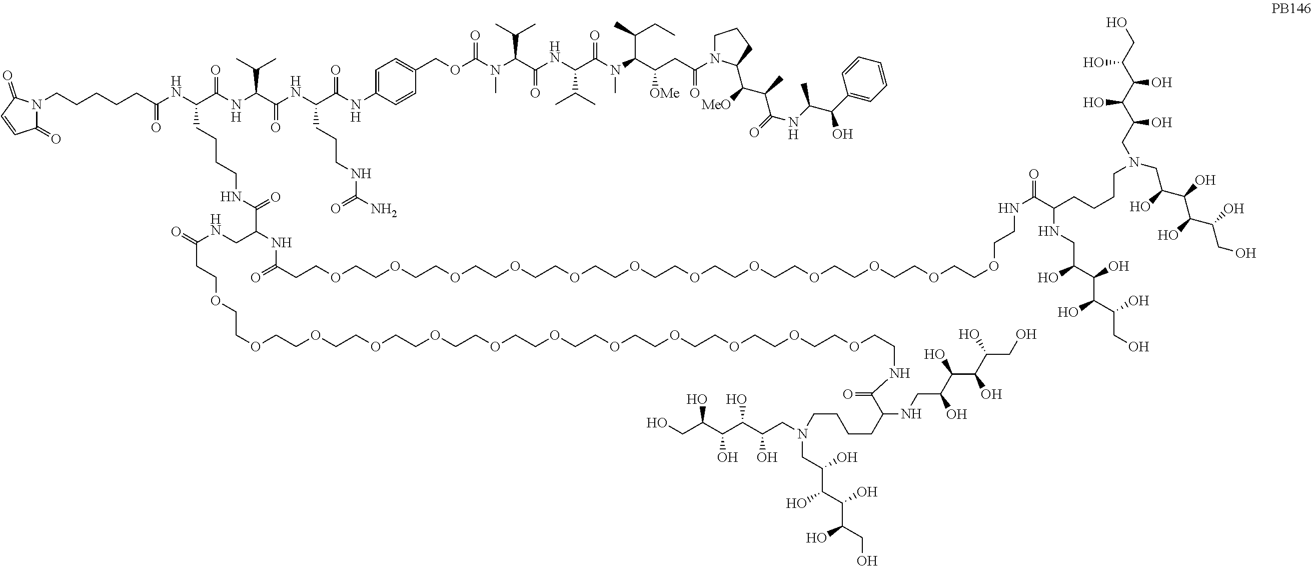

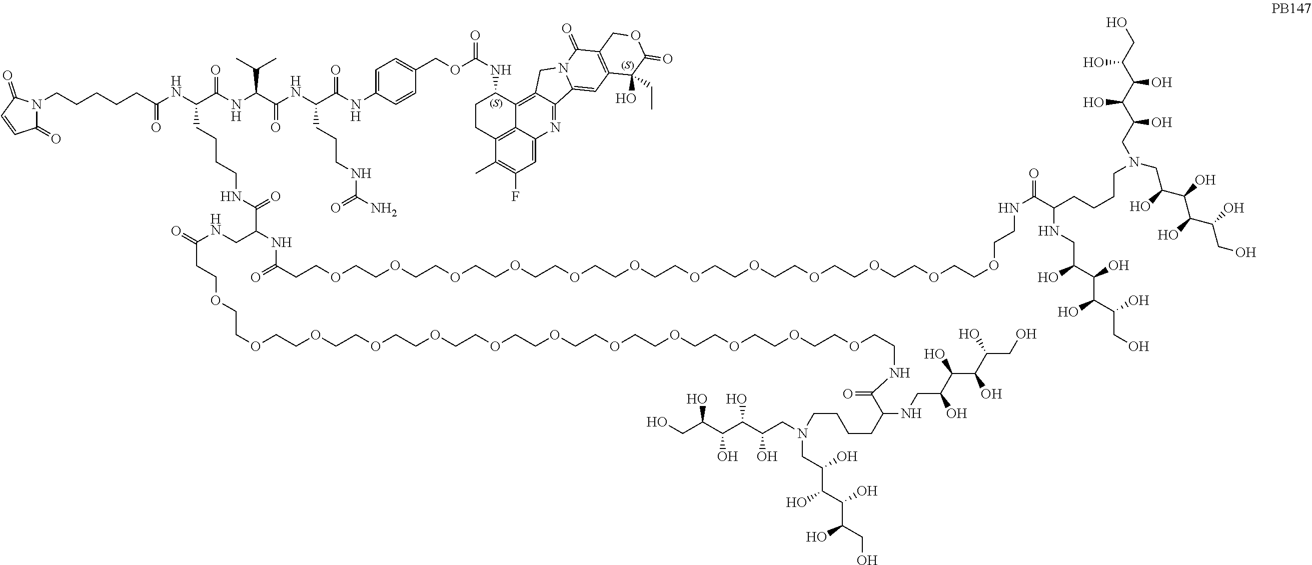

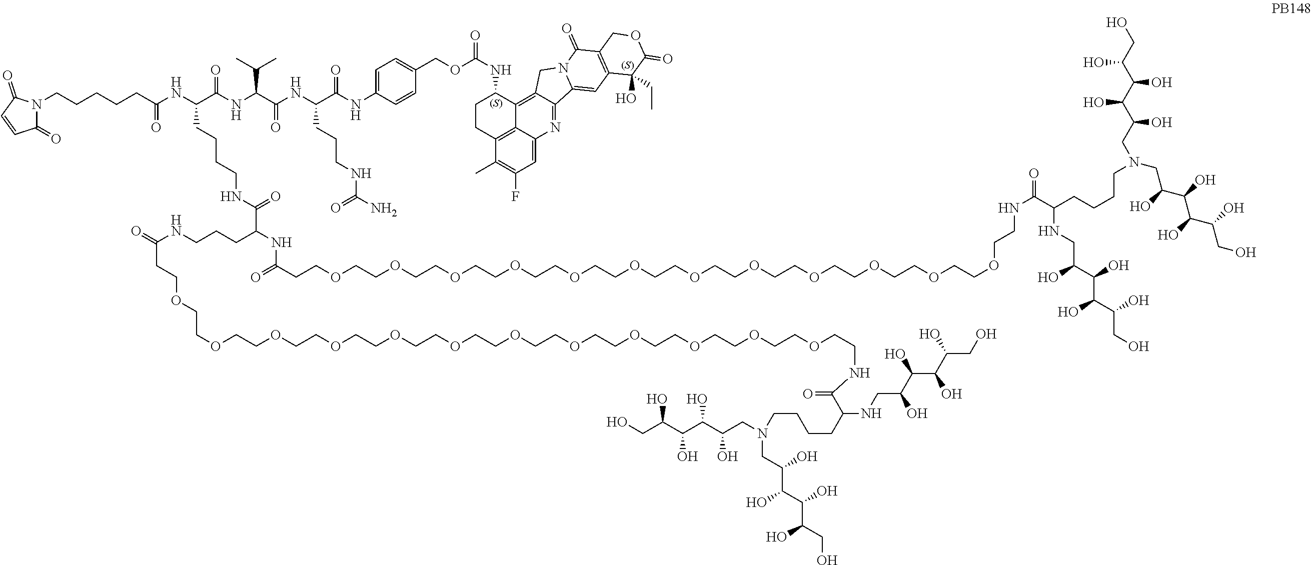

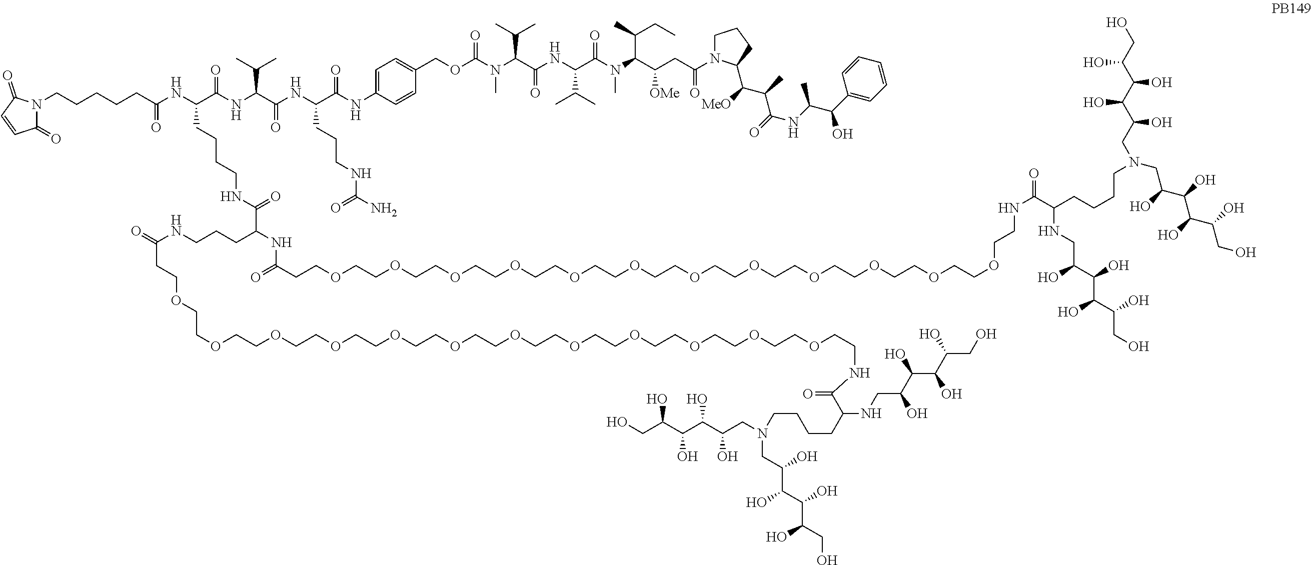

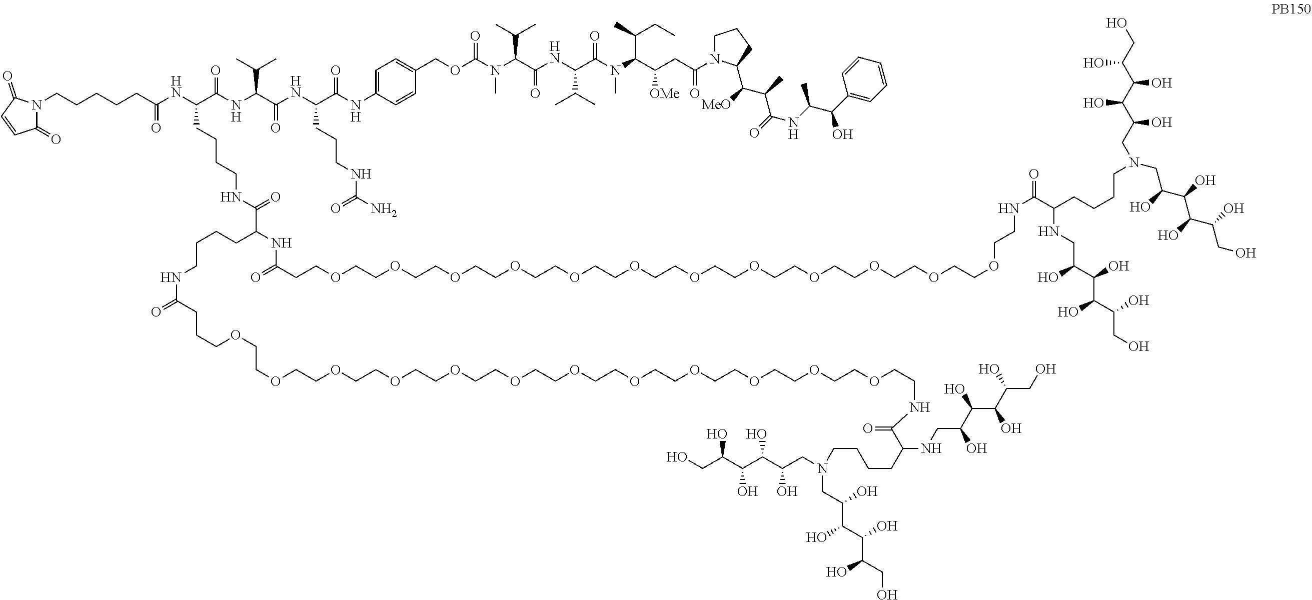

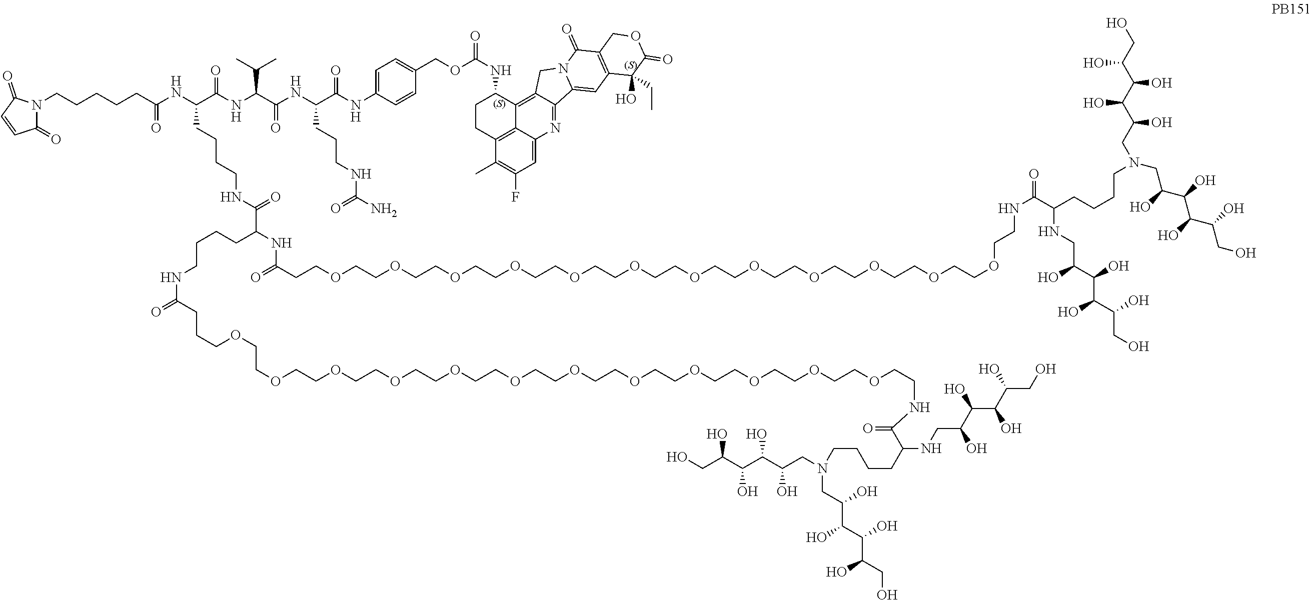

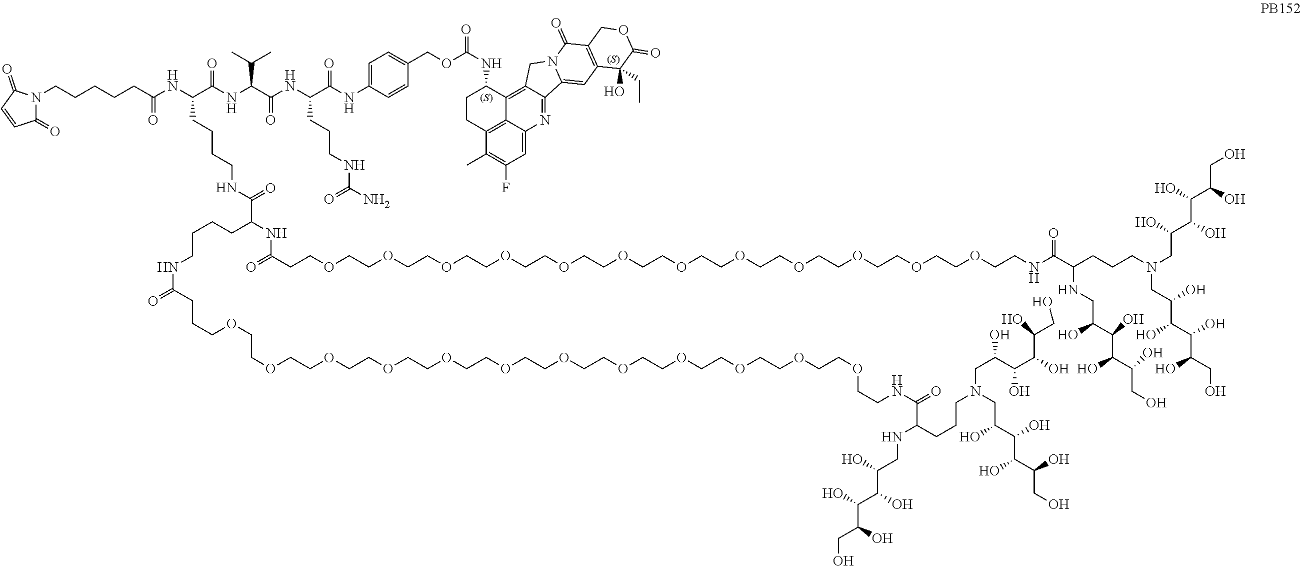

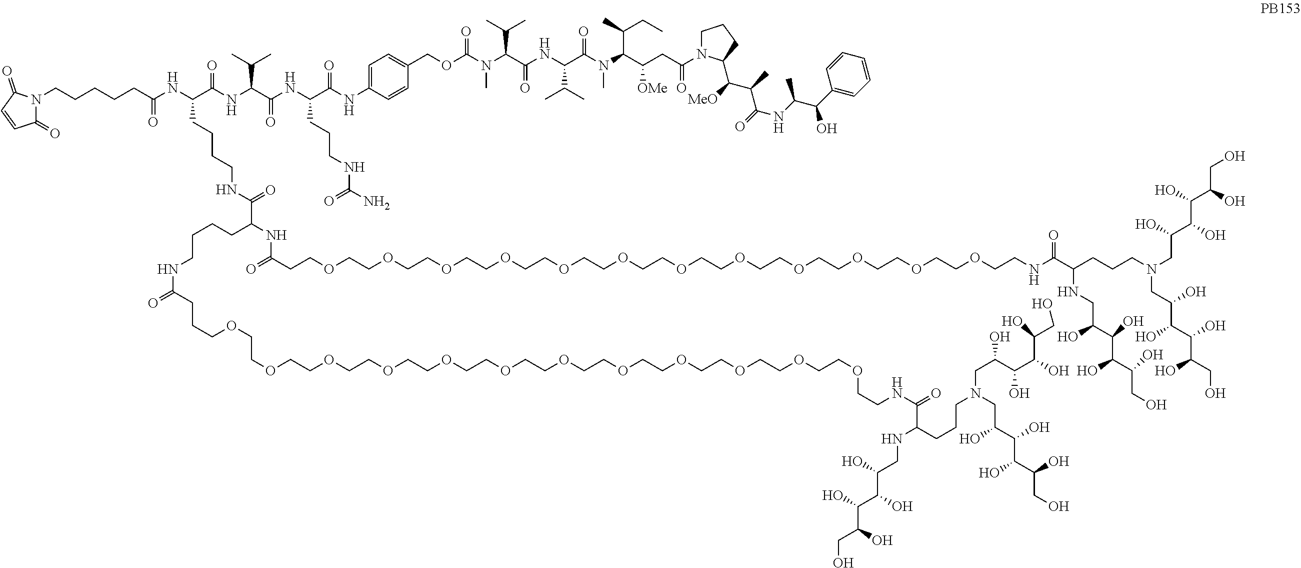

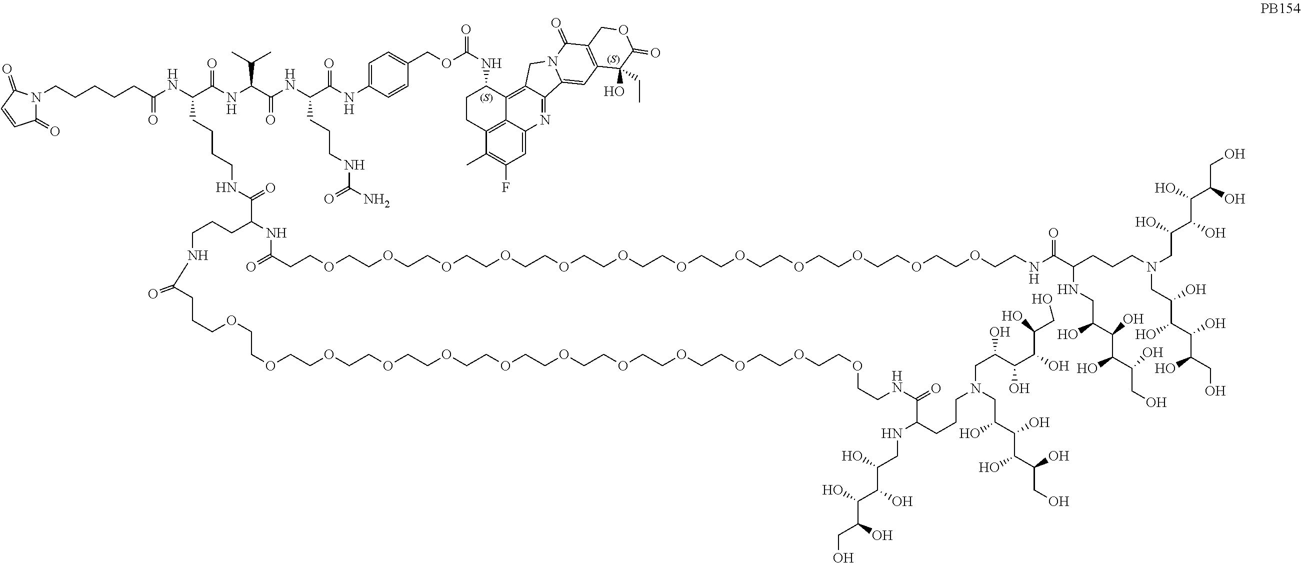

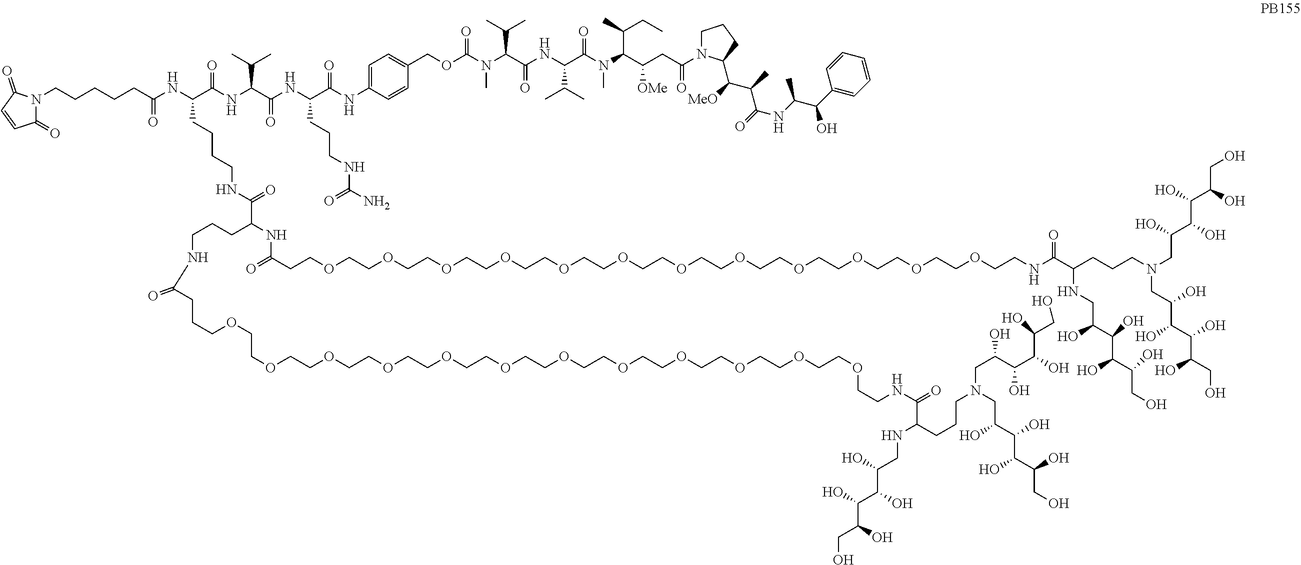

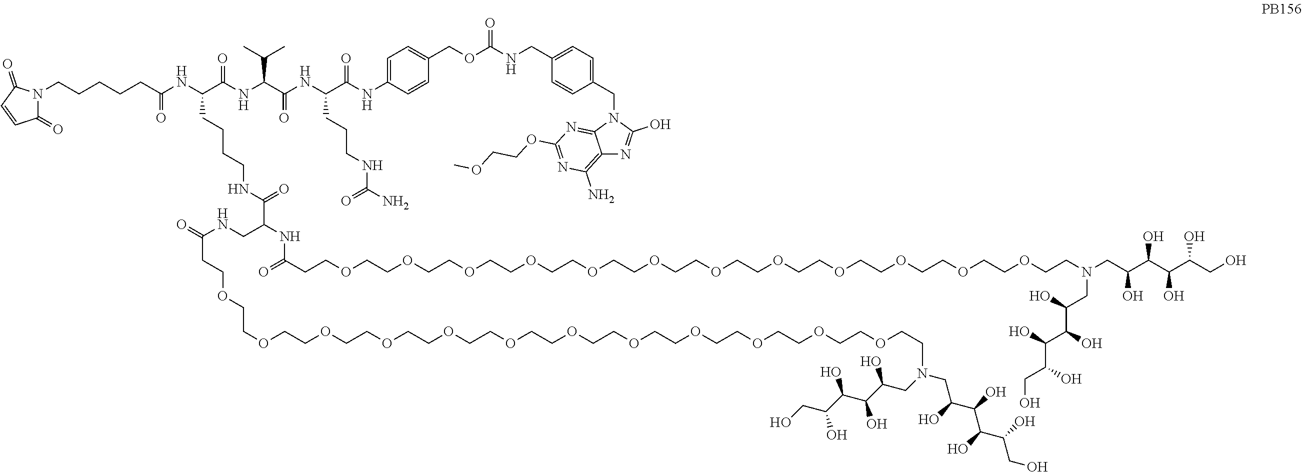

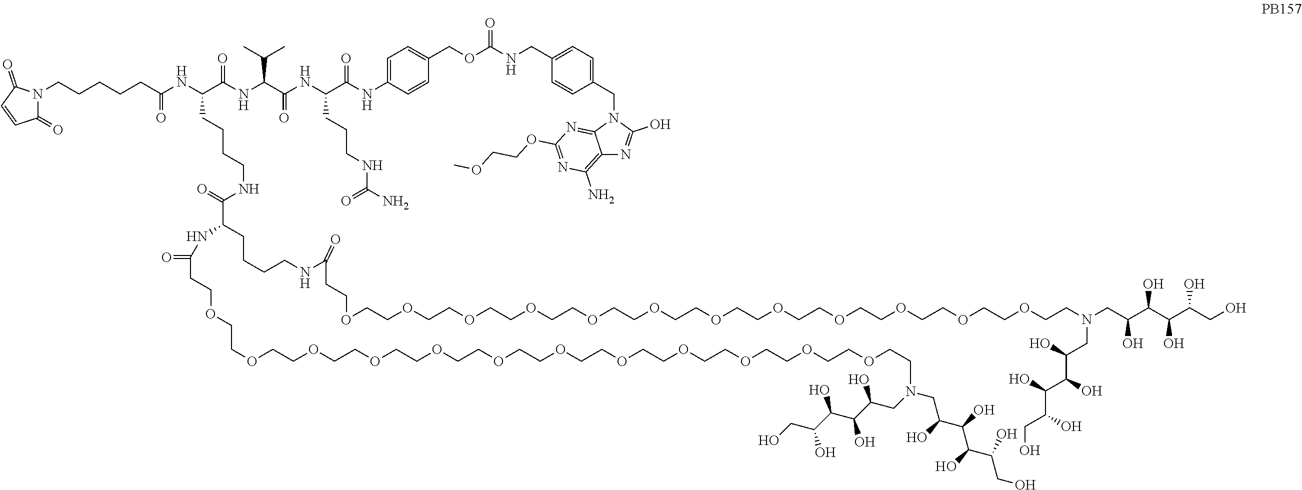

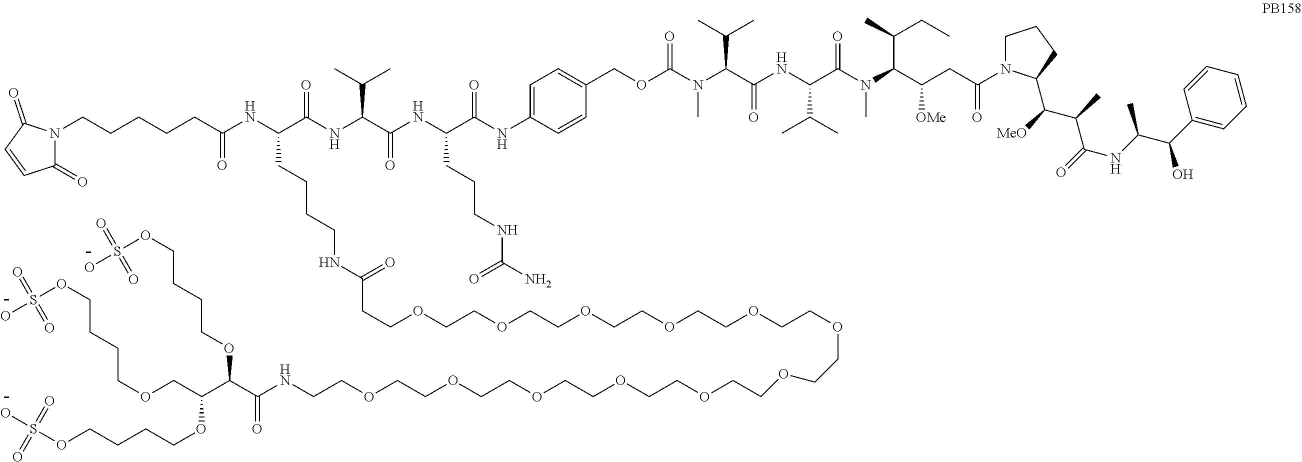

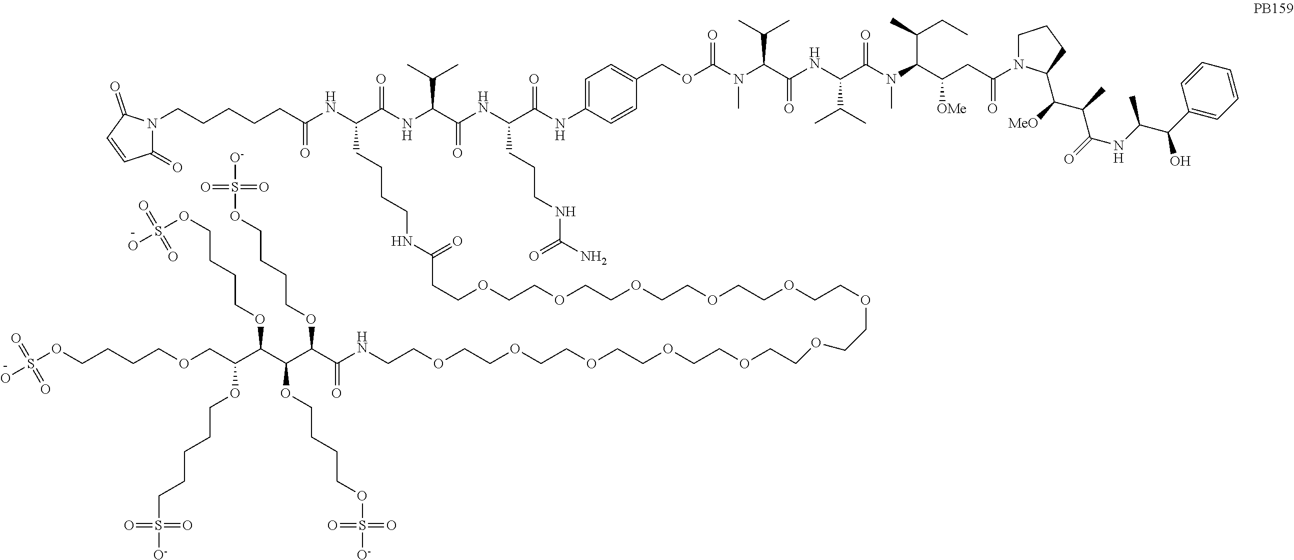

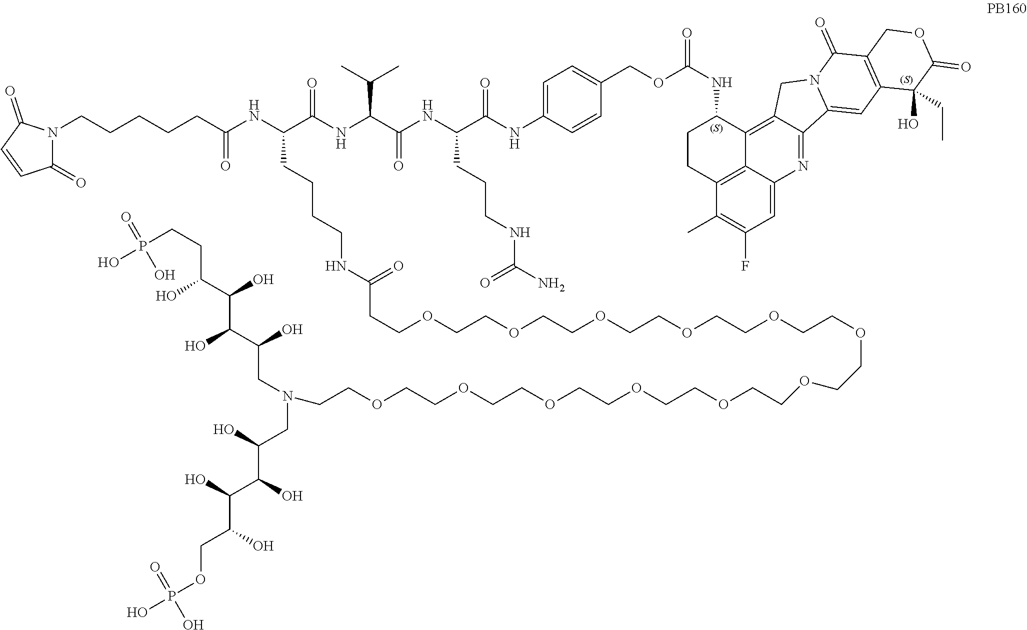

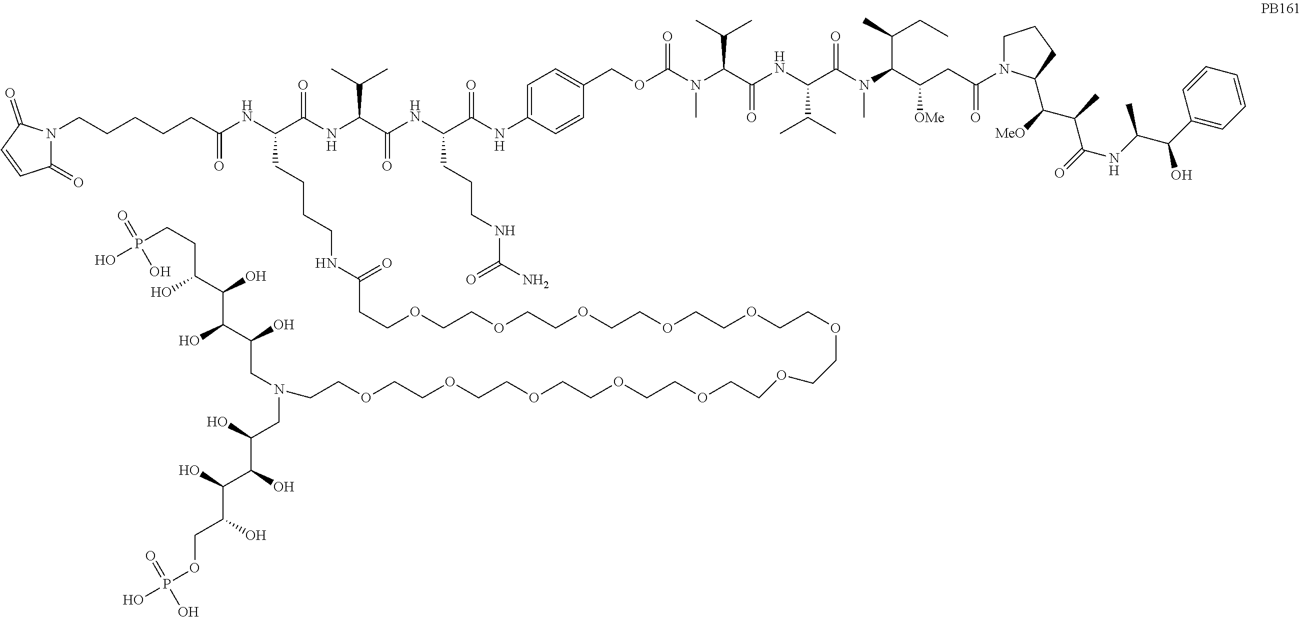

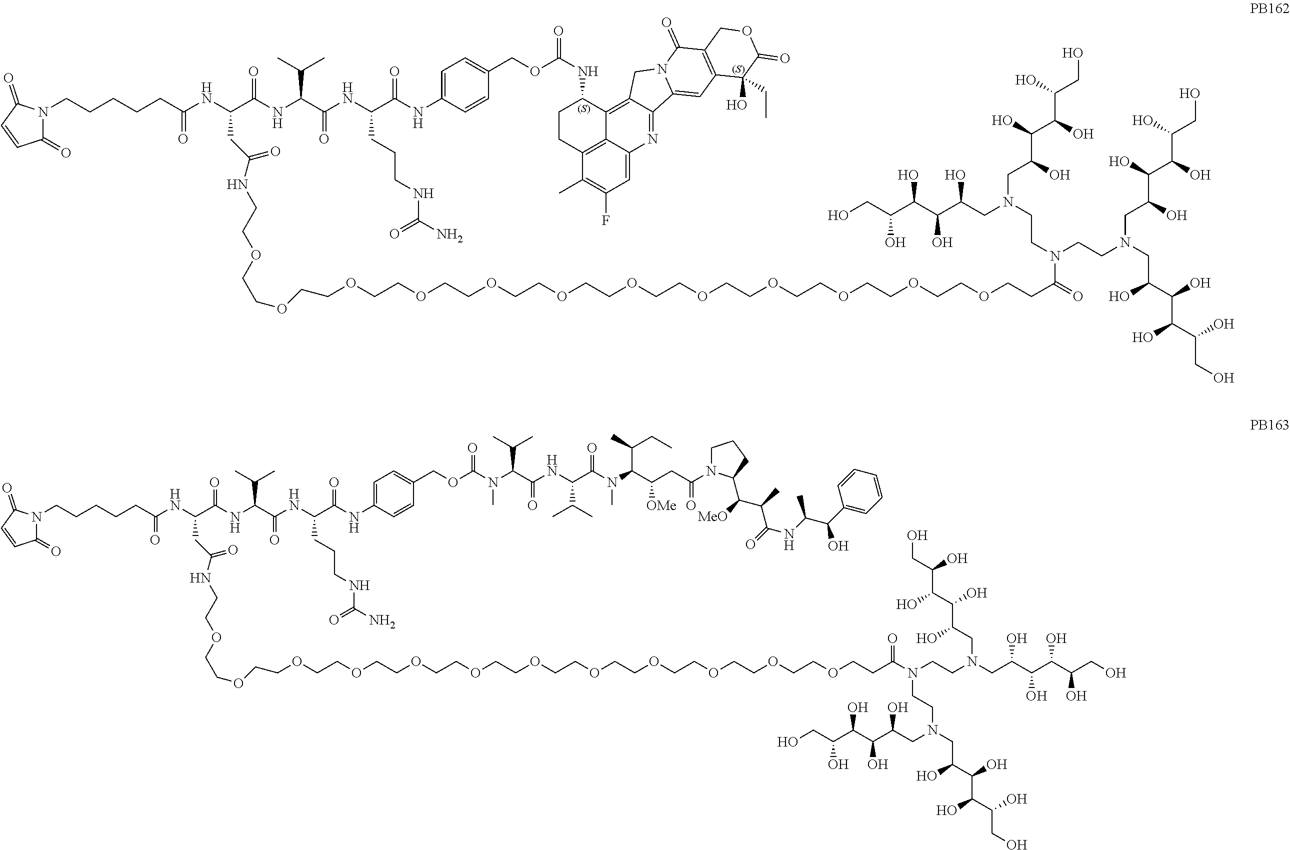

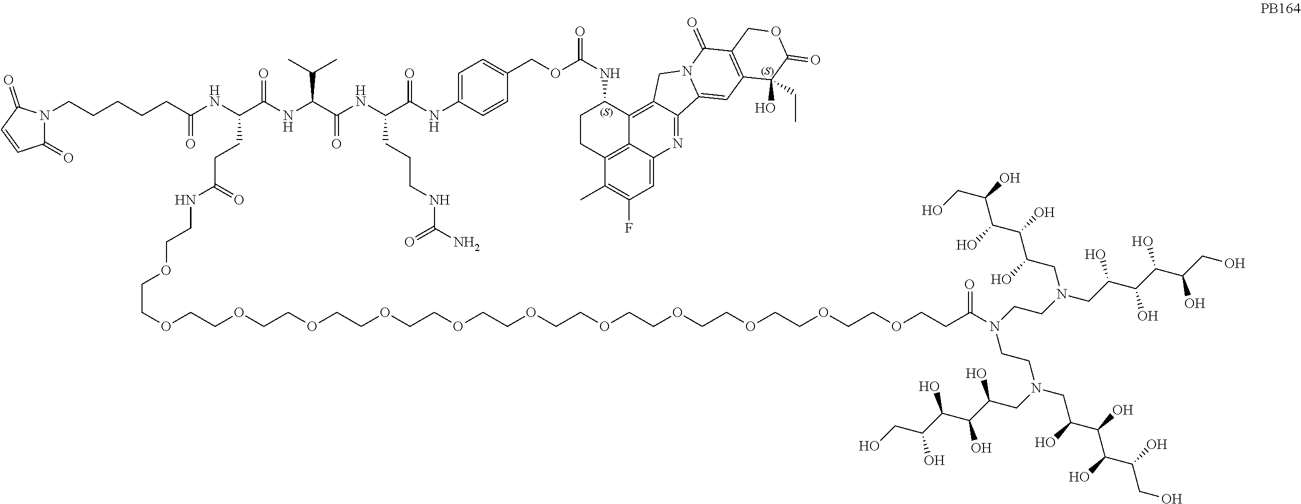

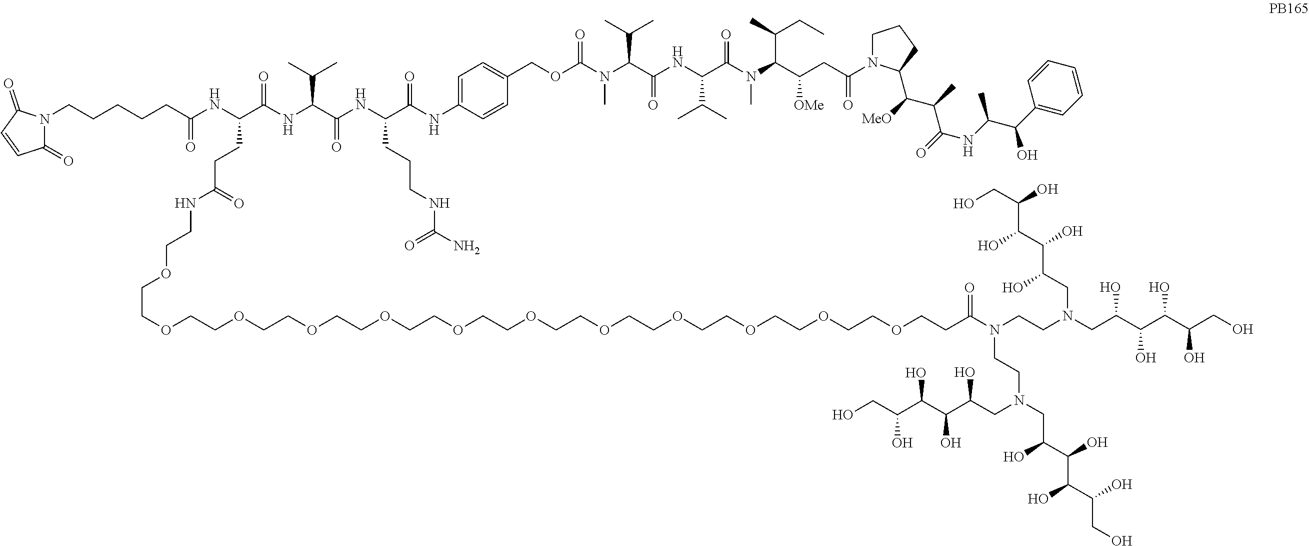

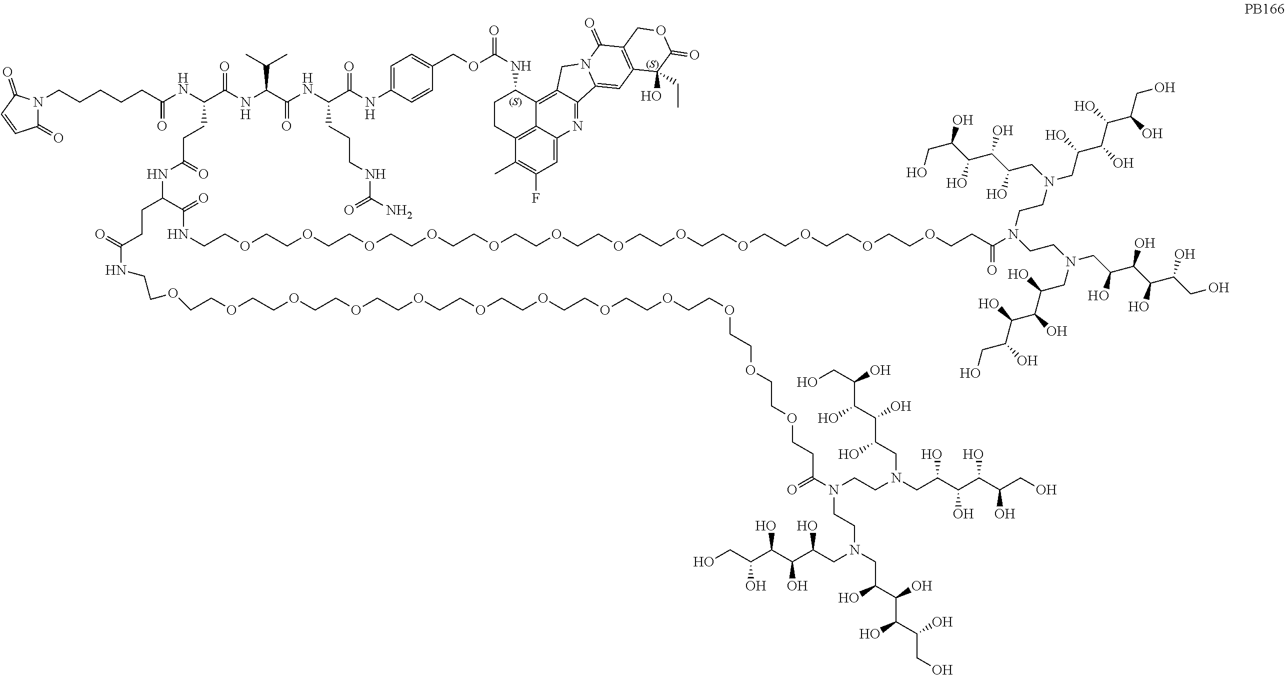

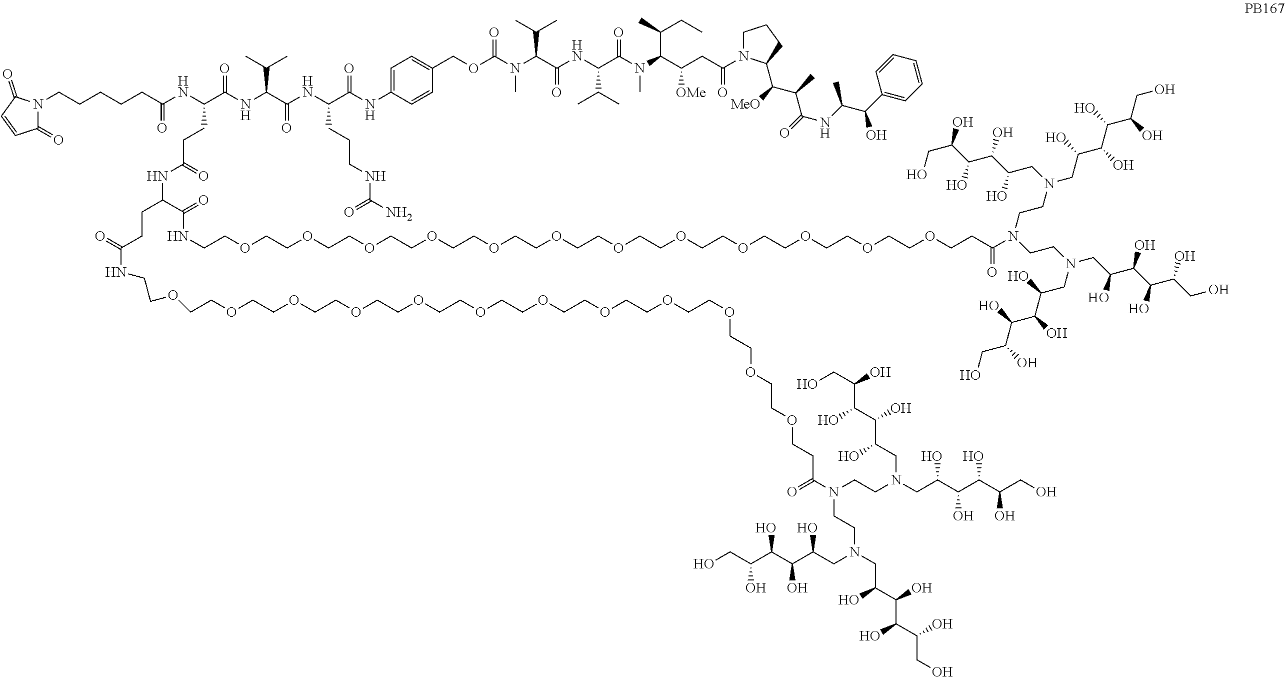

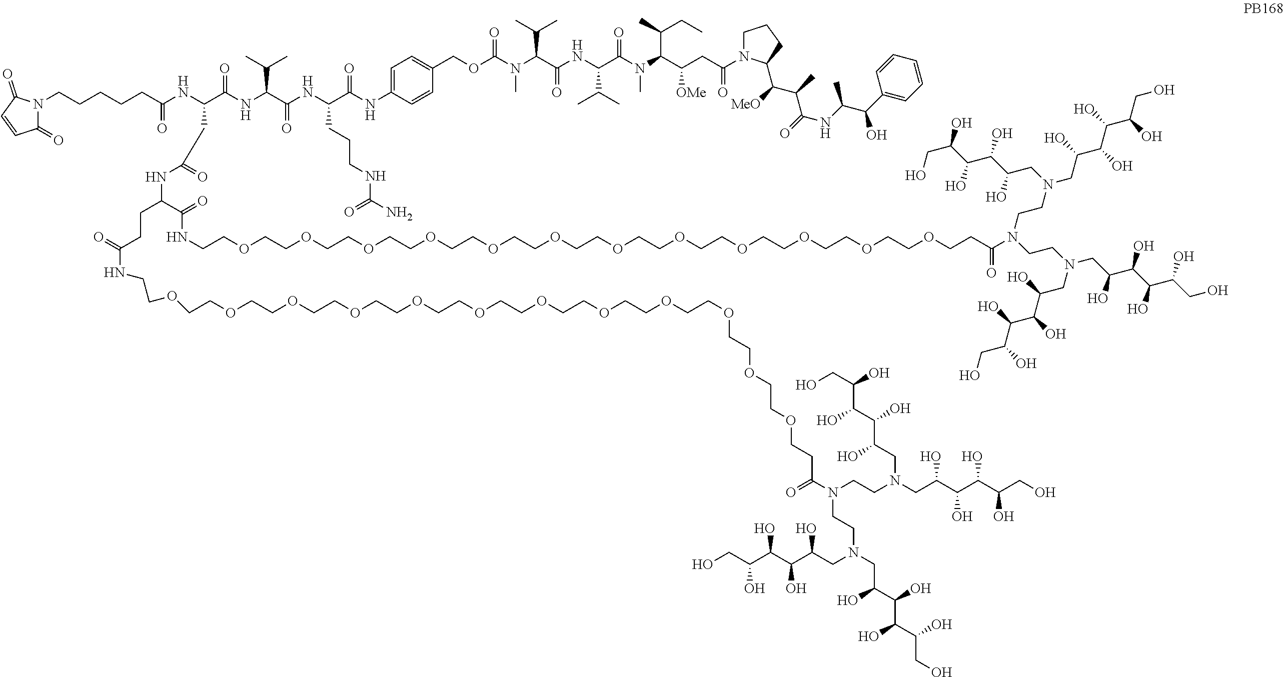

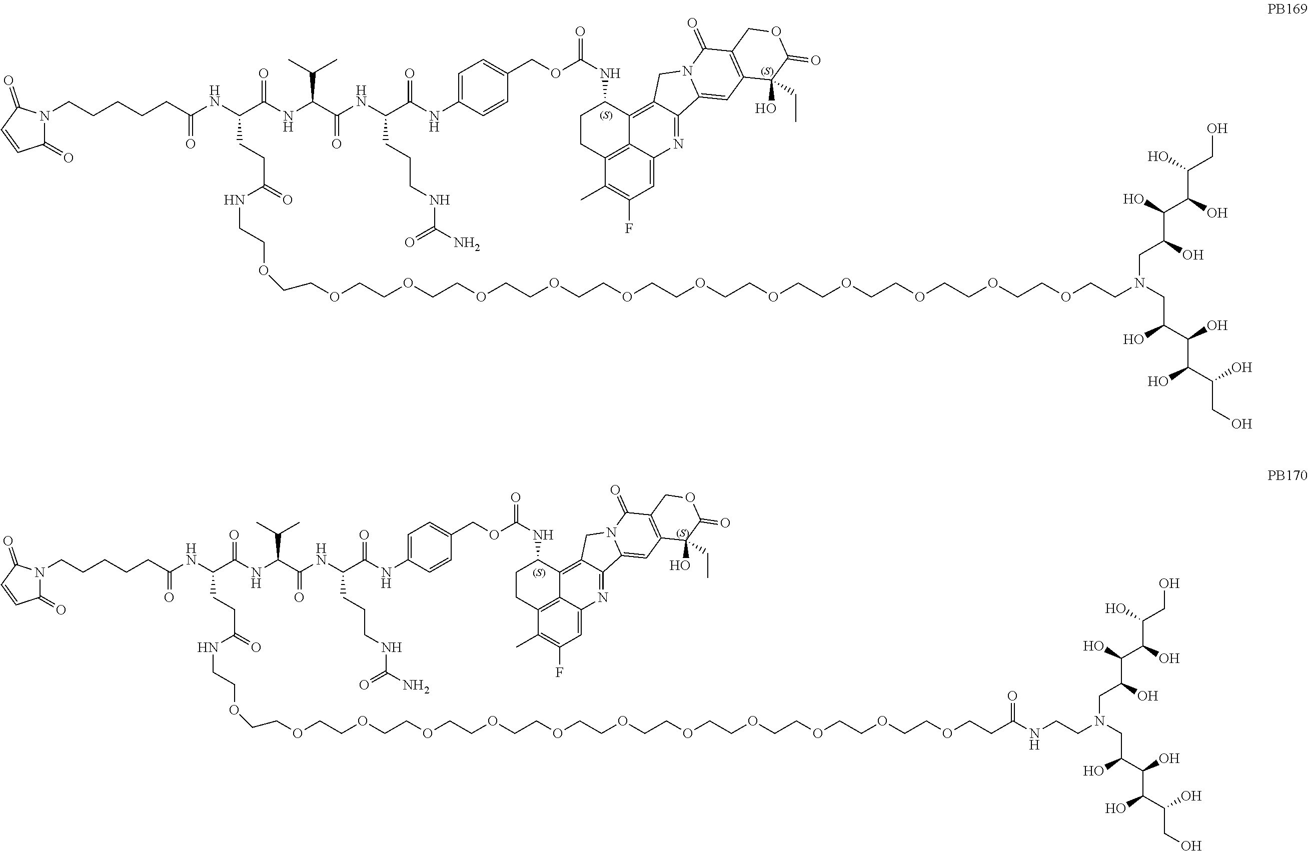

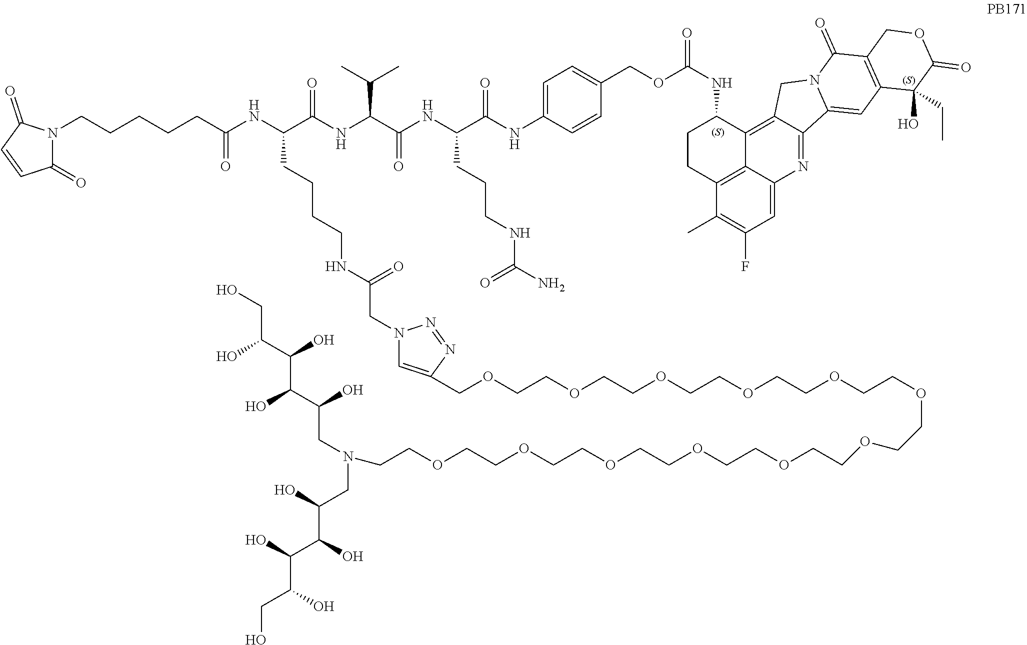

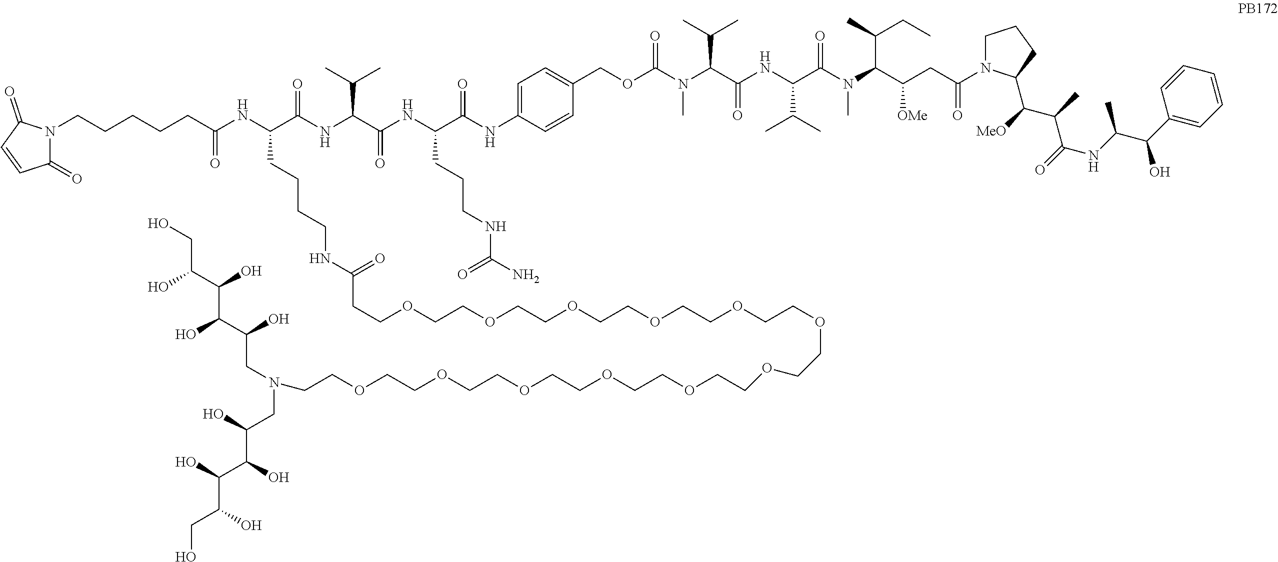

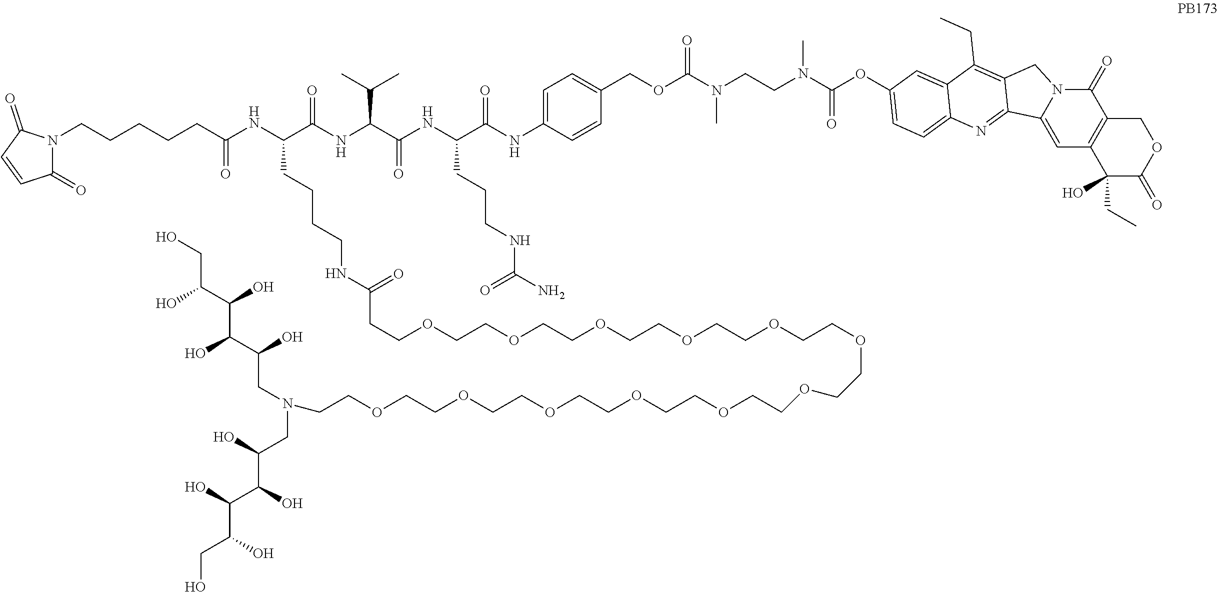

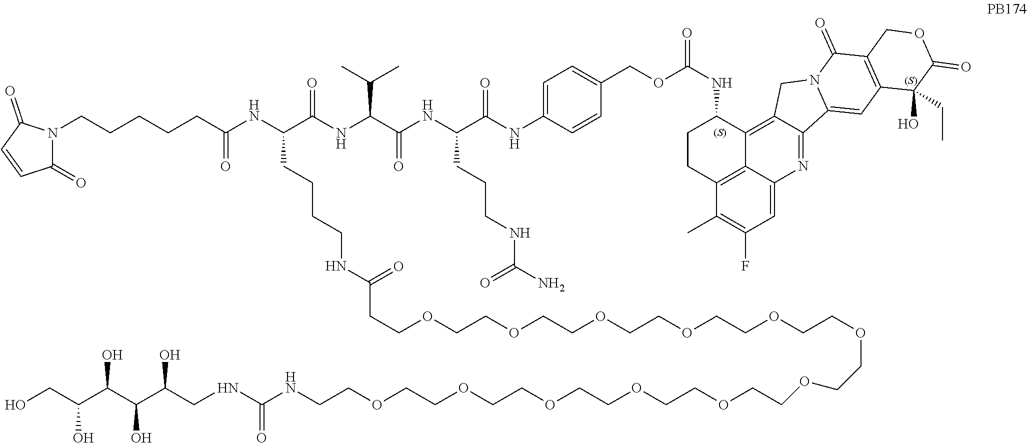

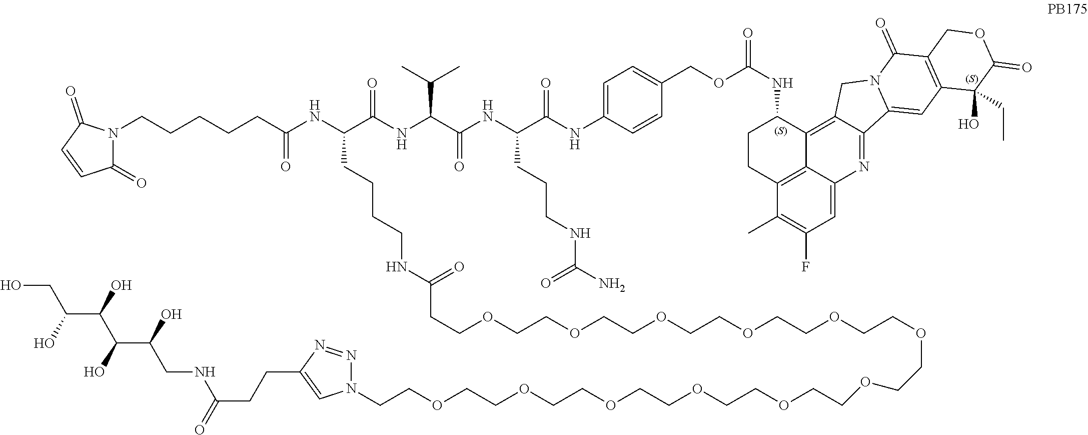

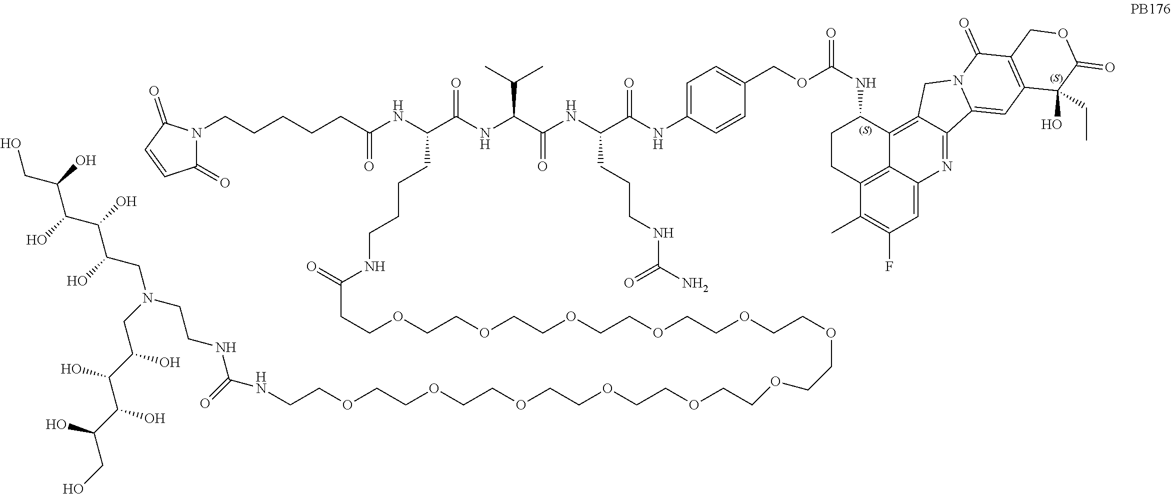

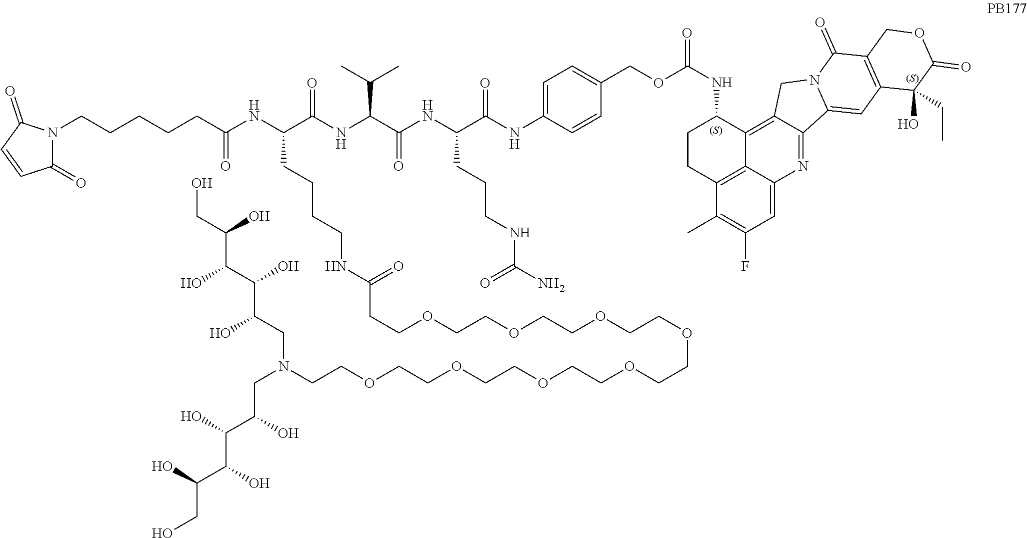

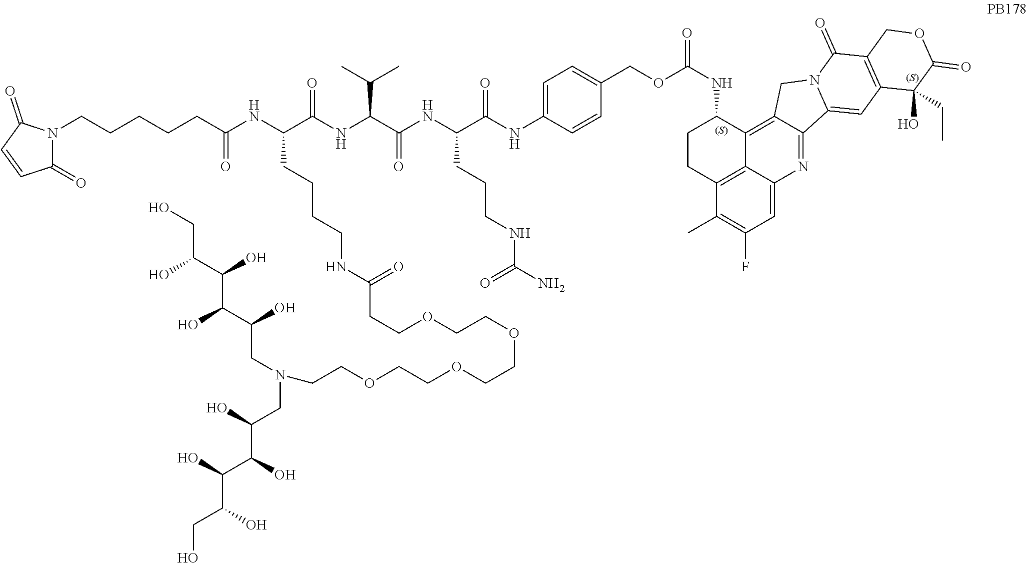

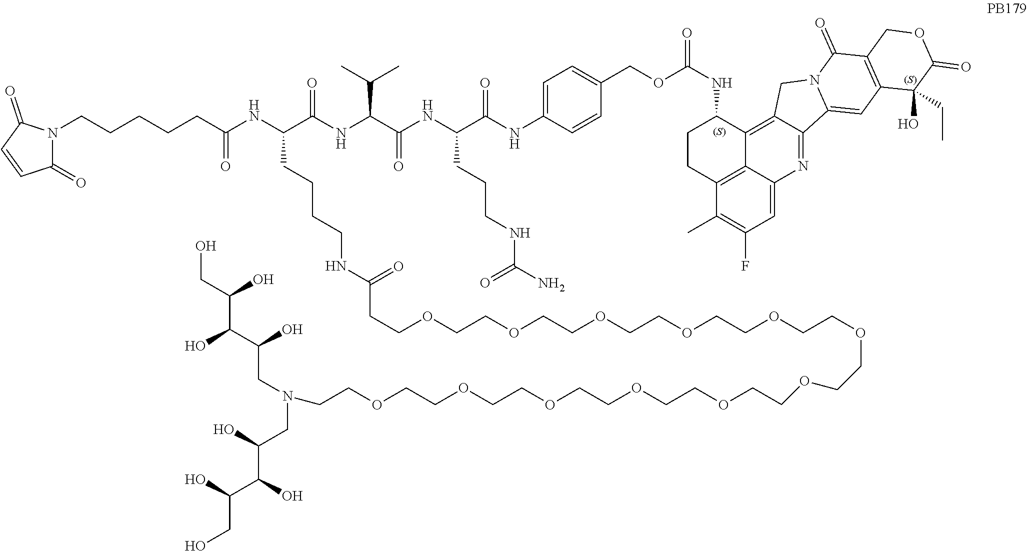

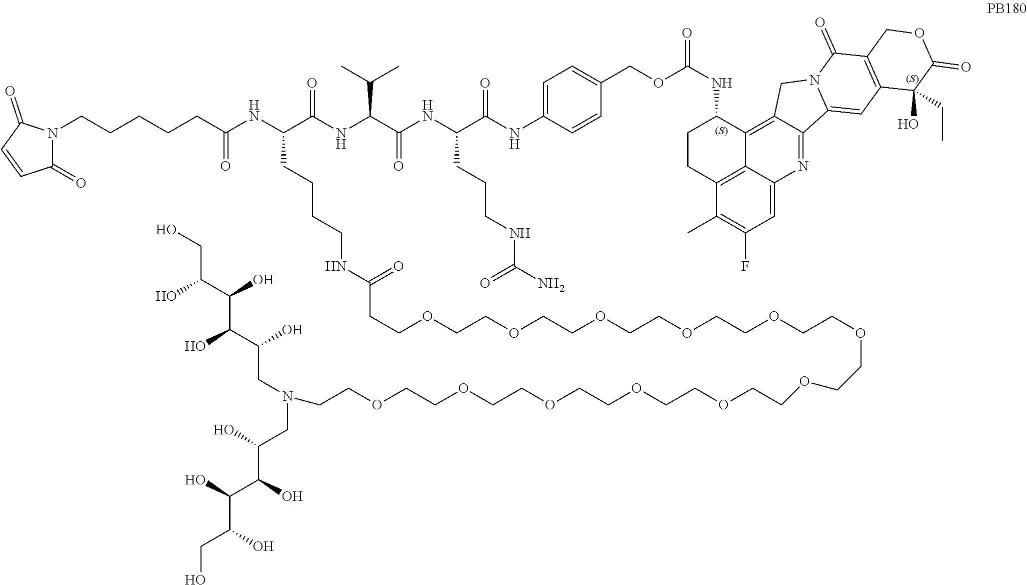

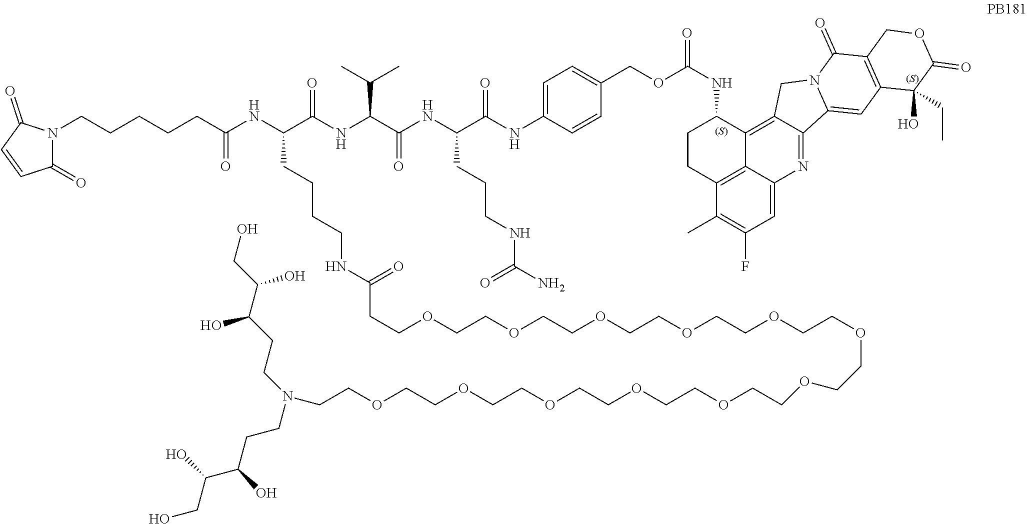

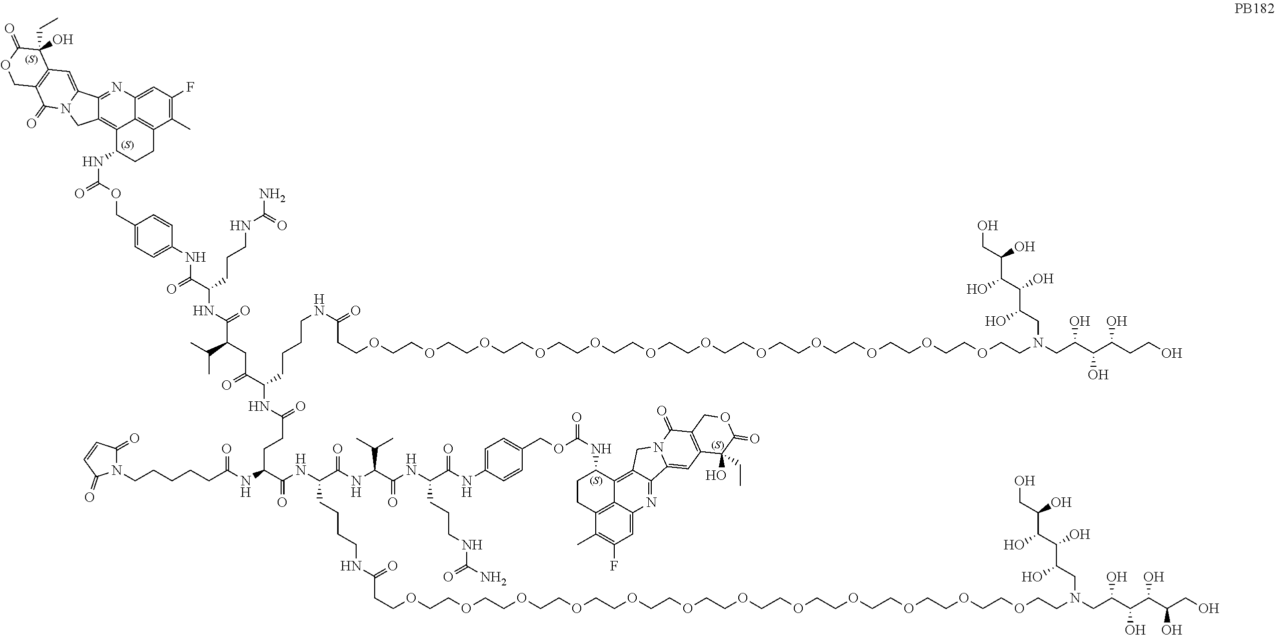

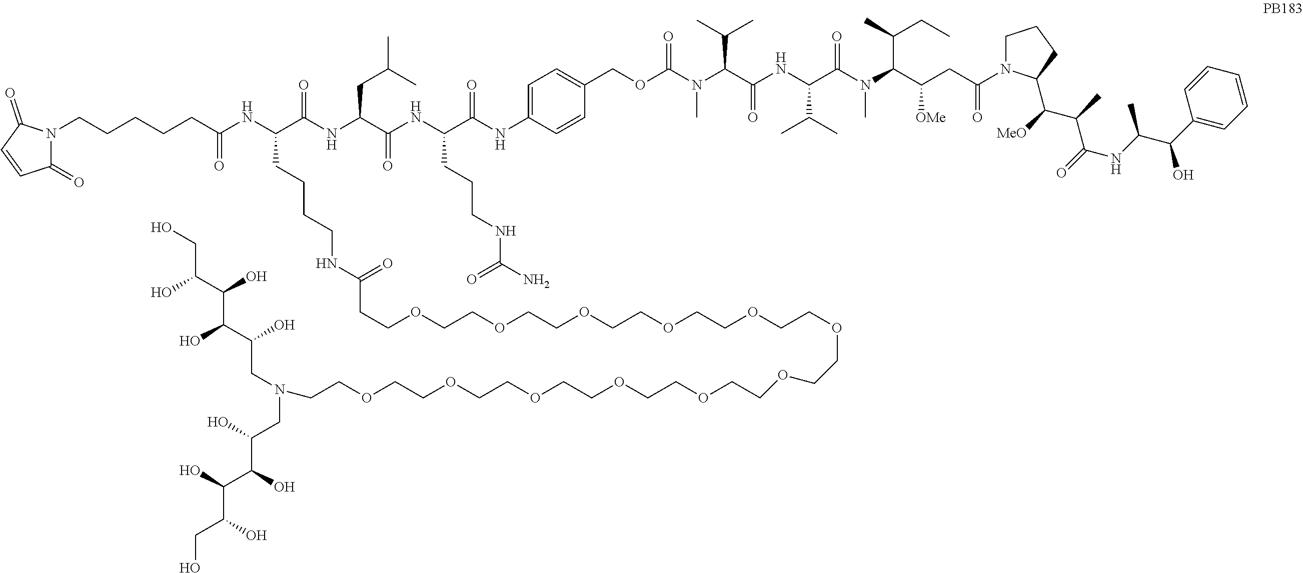

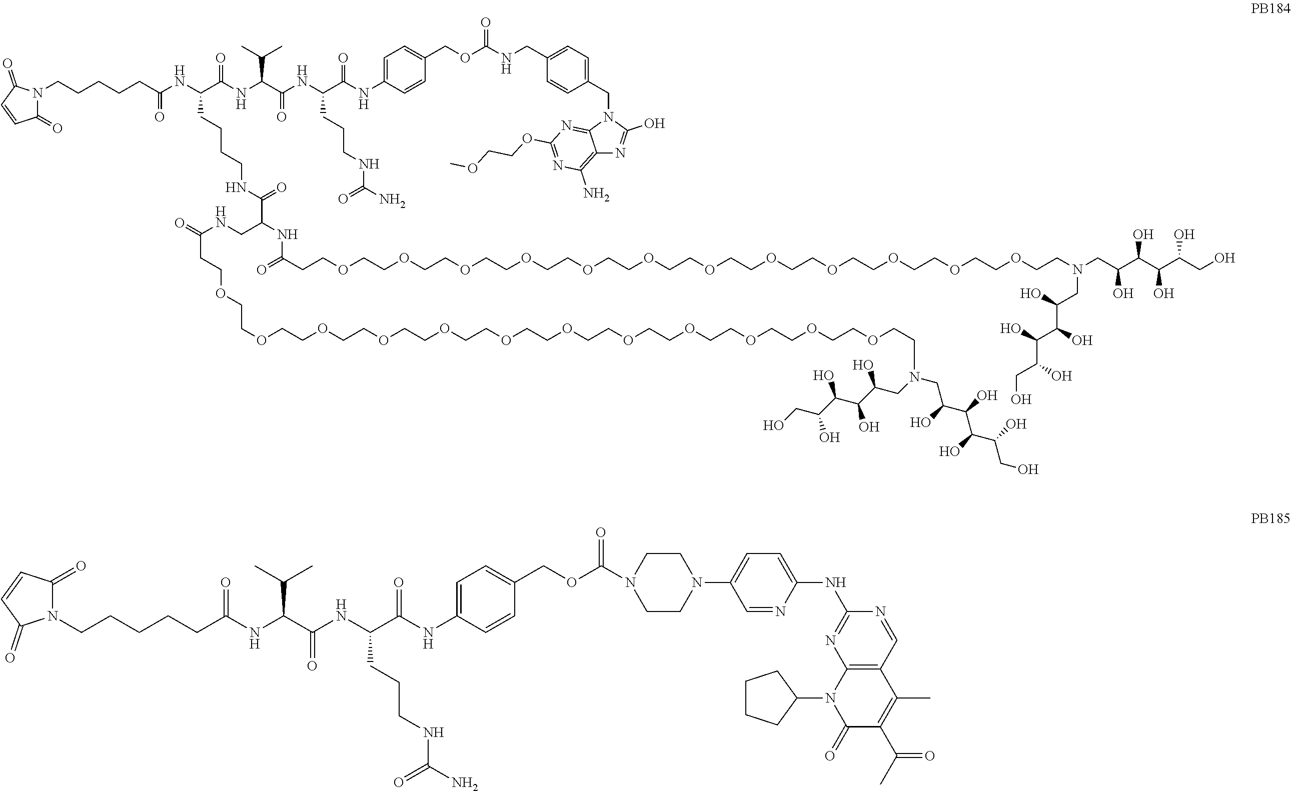

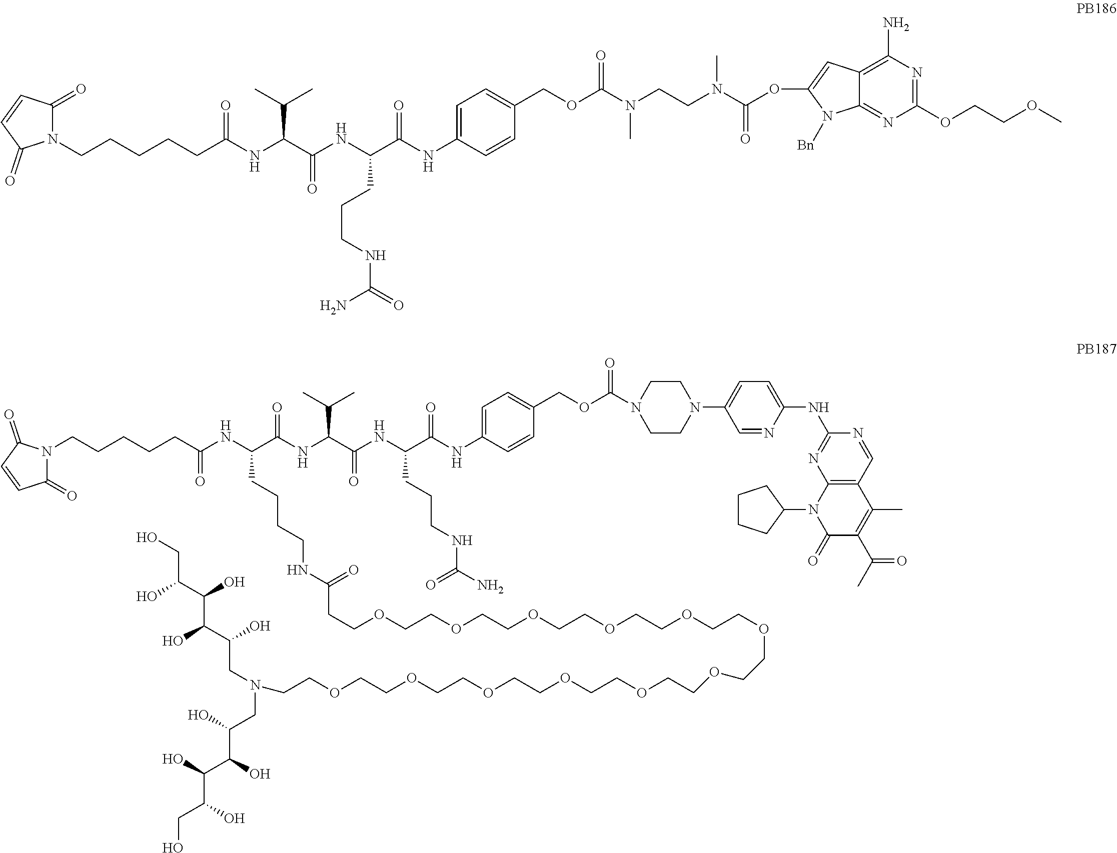

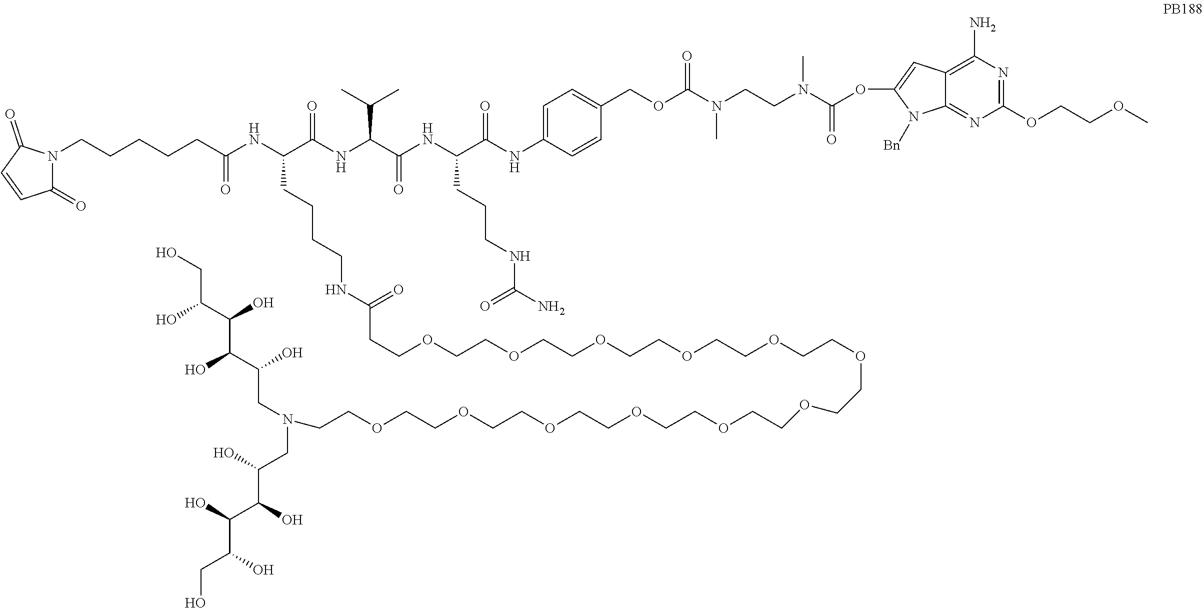

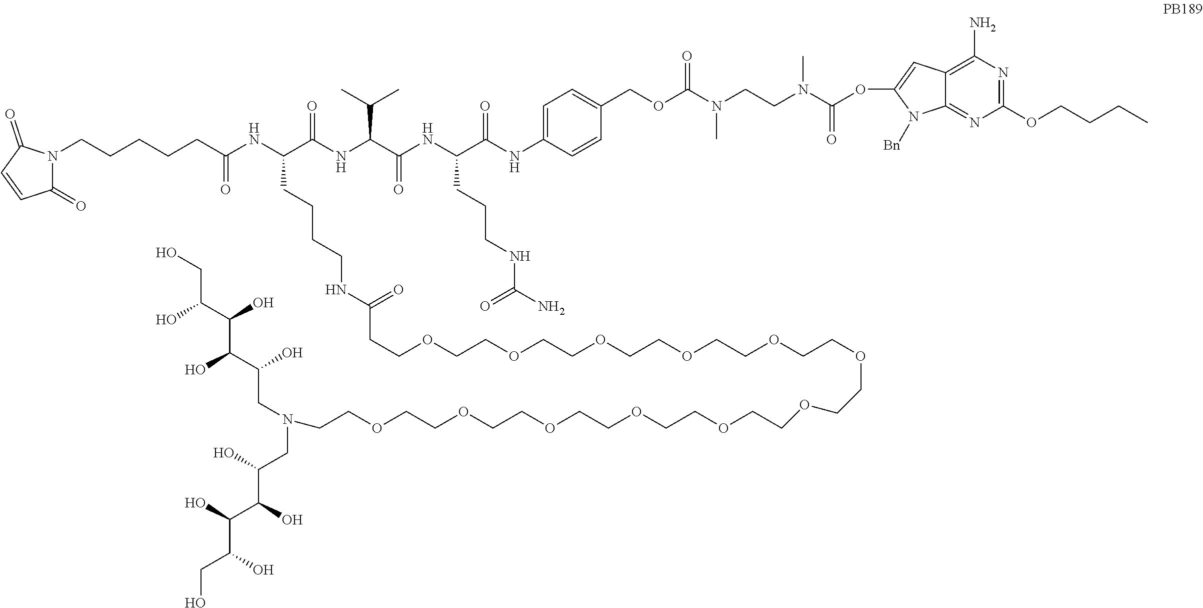

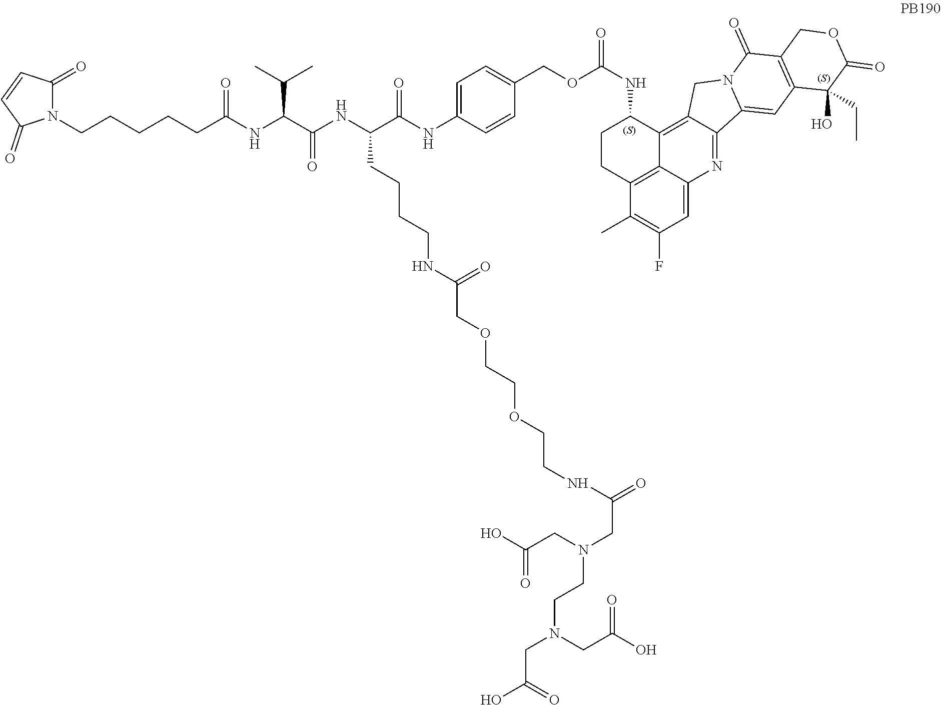

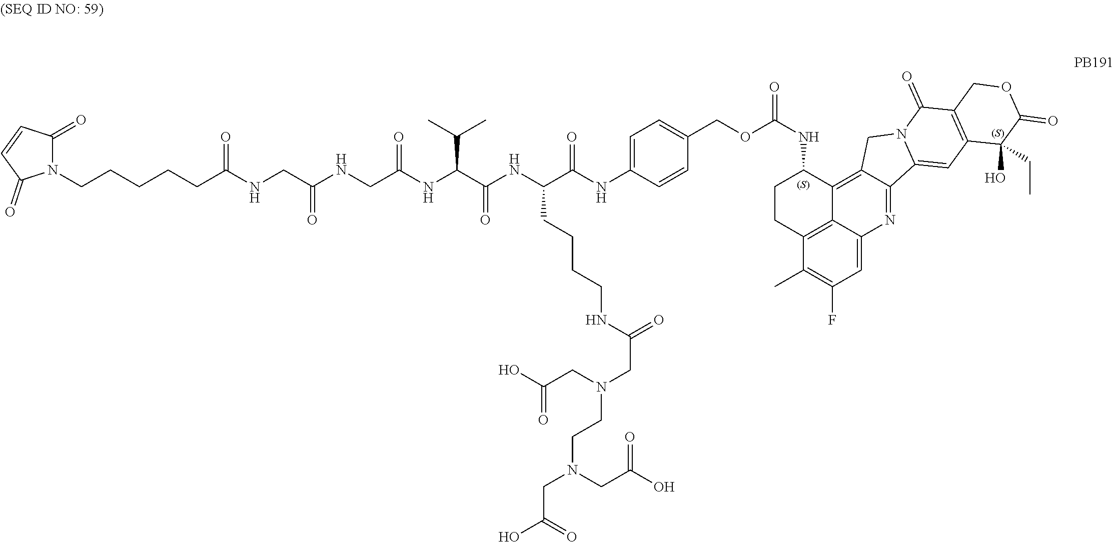

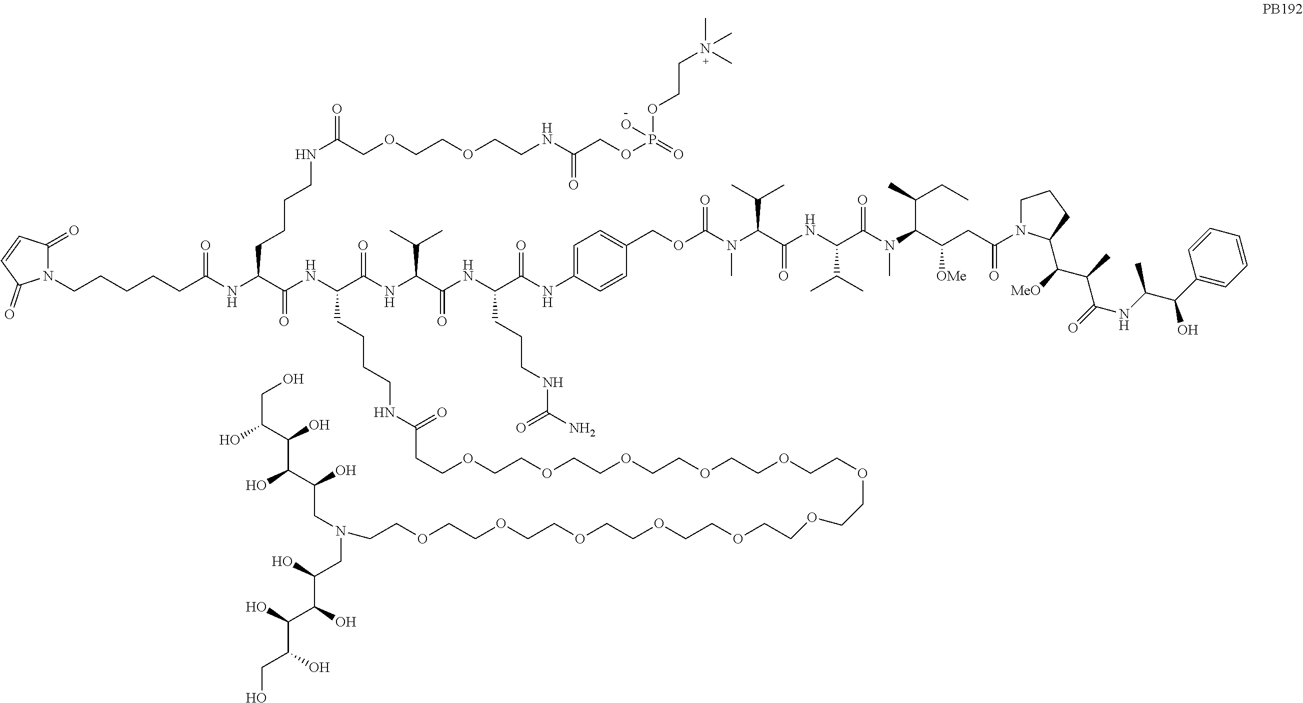

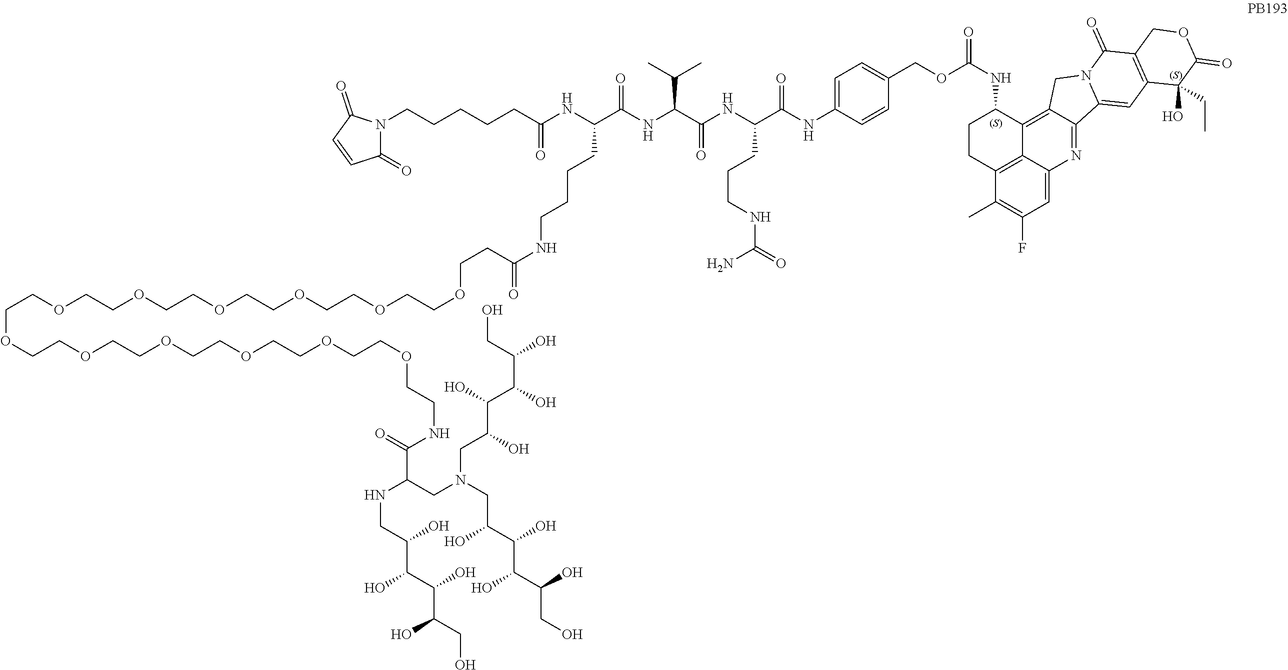

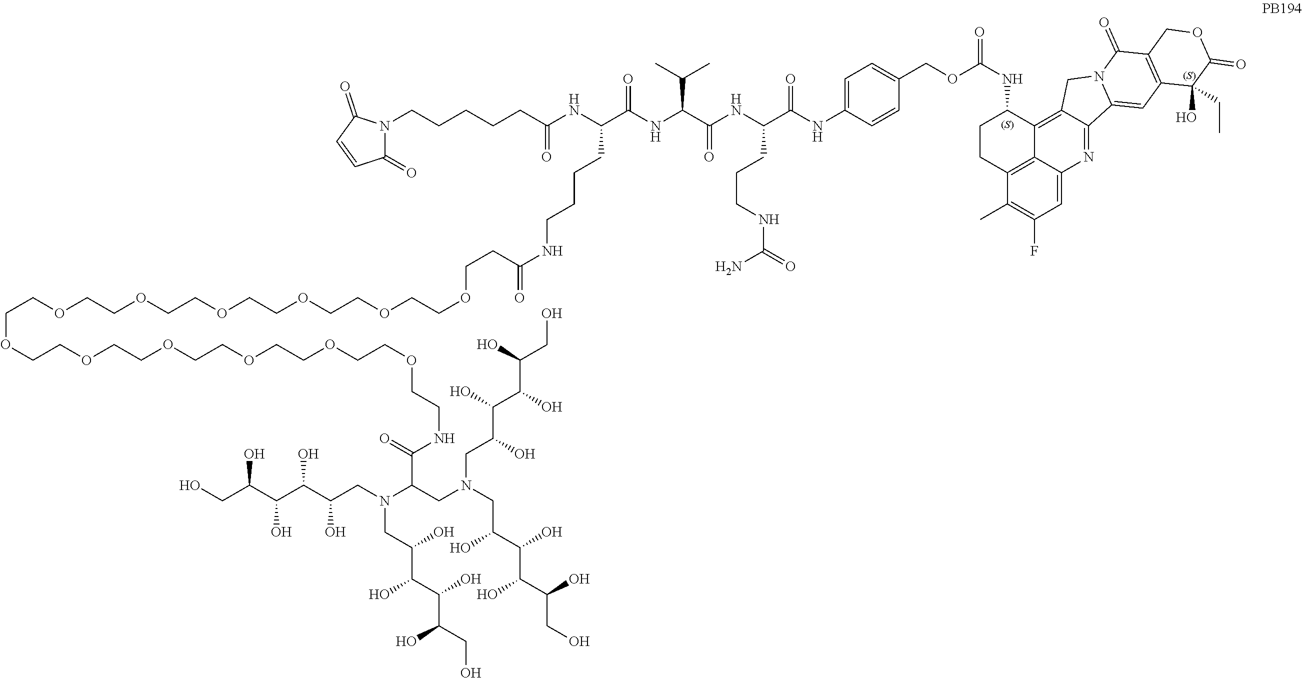

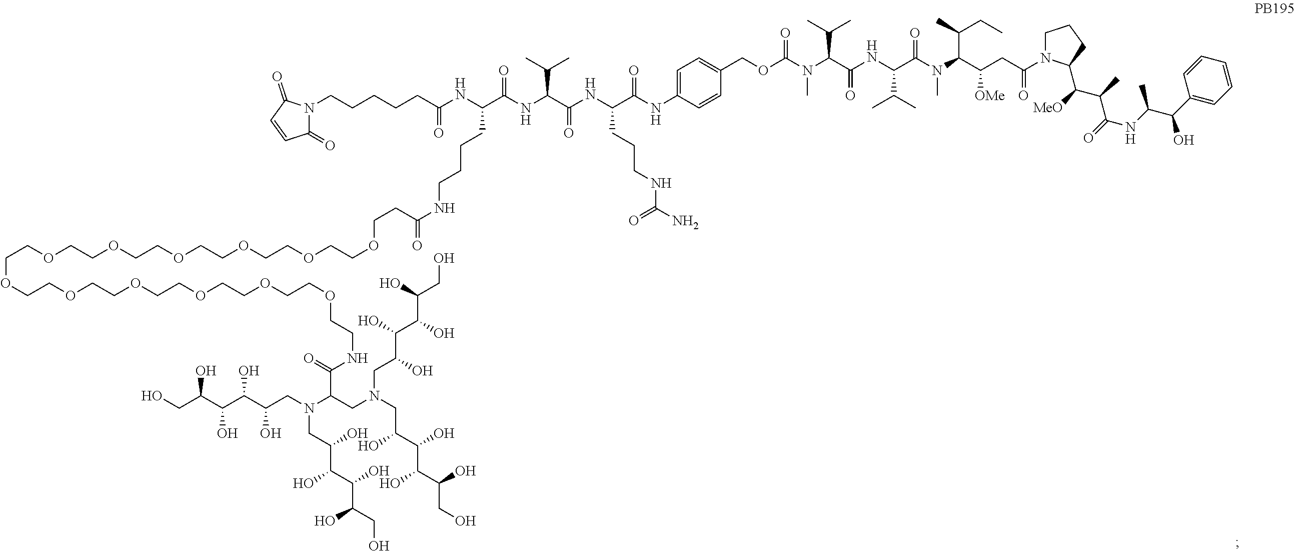

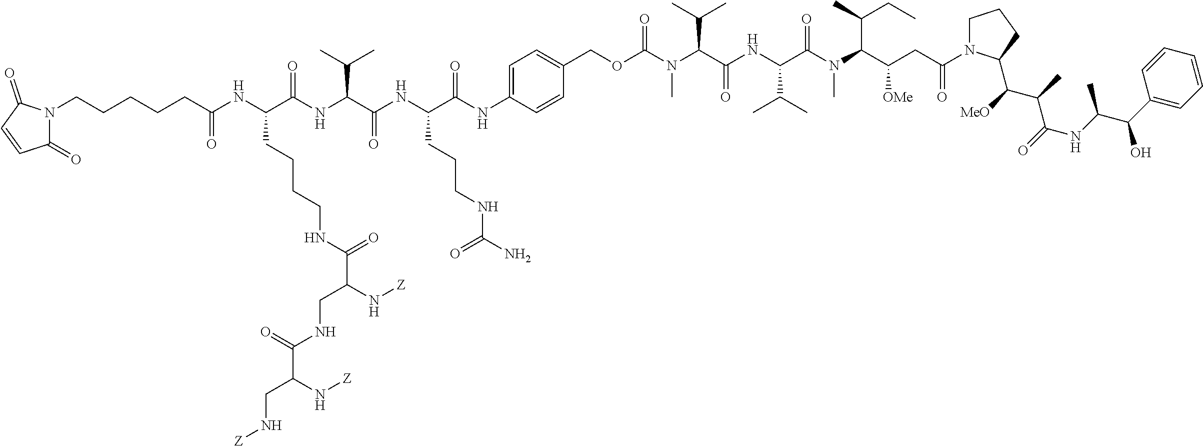

wherein each Z is attached at * and is individually selected from:

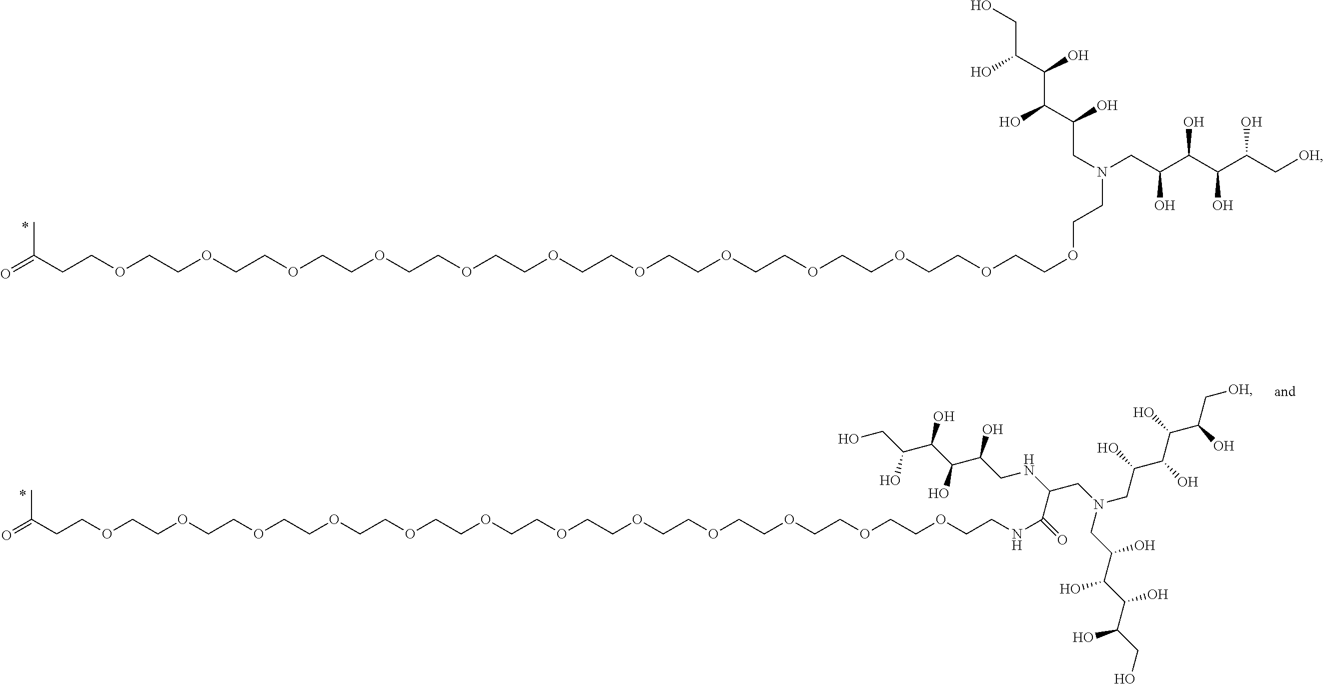

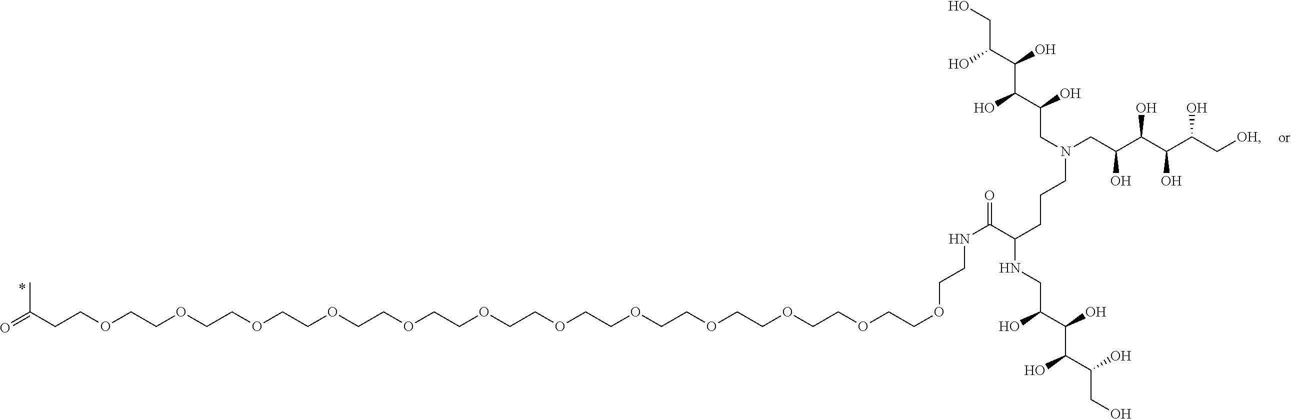

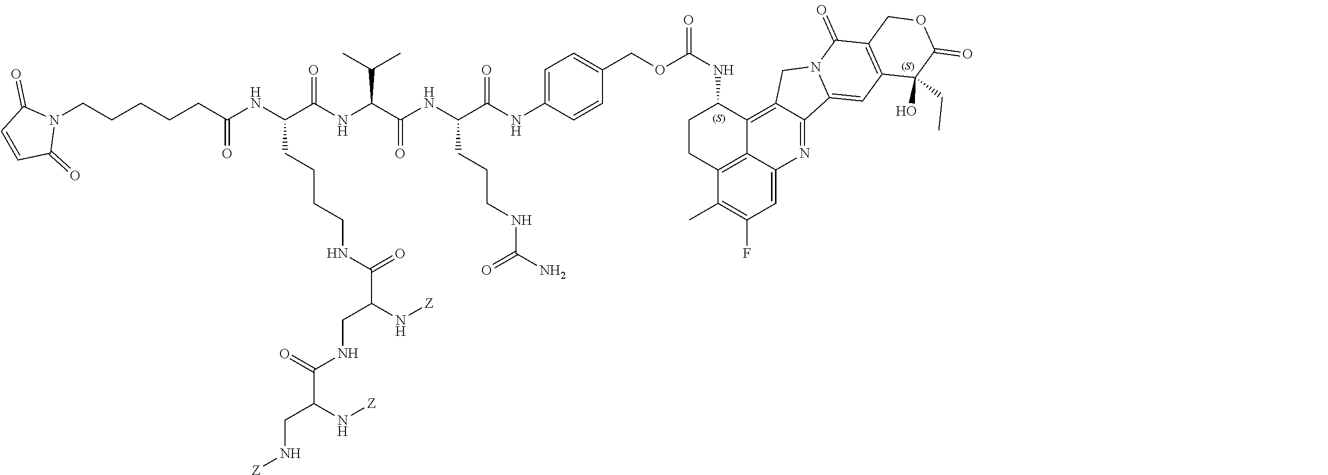

wherein each Z is attached at * and is individually selected from:

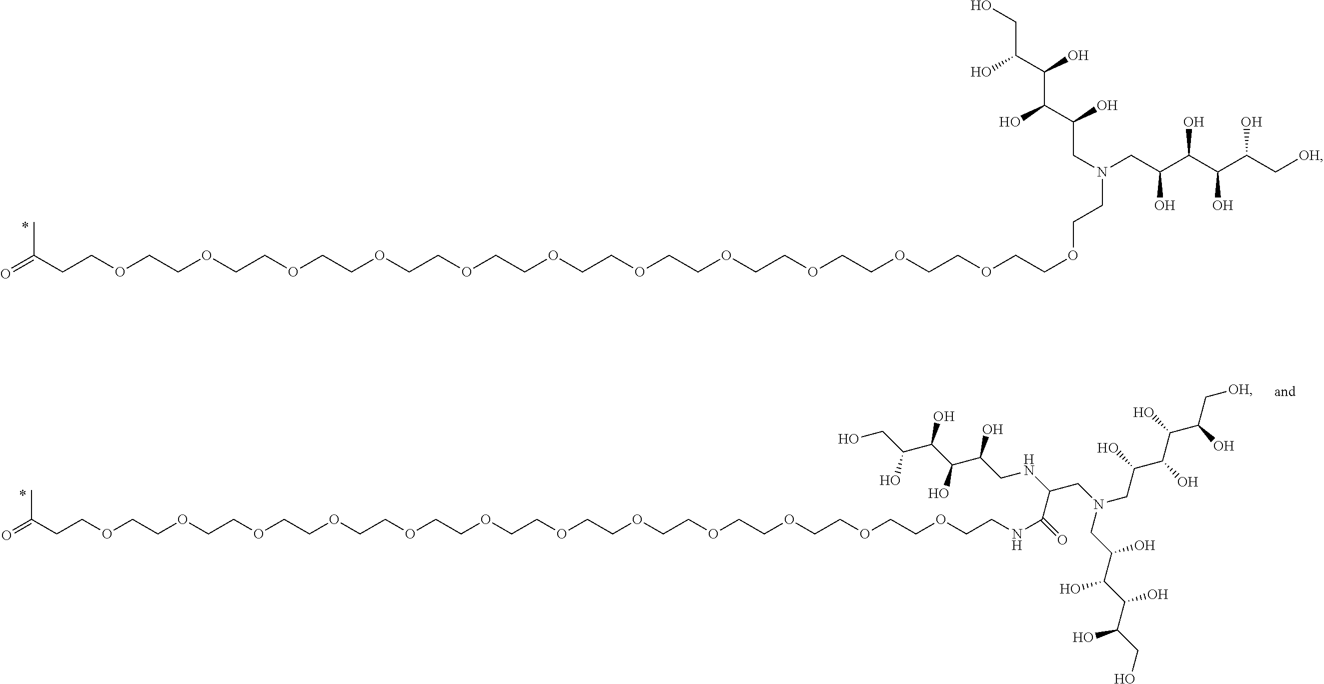 [SMILES: CC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO ; CC(=O)CCOCCOCCOCCOCCOCCOCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]

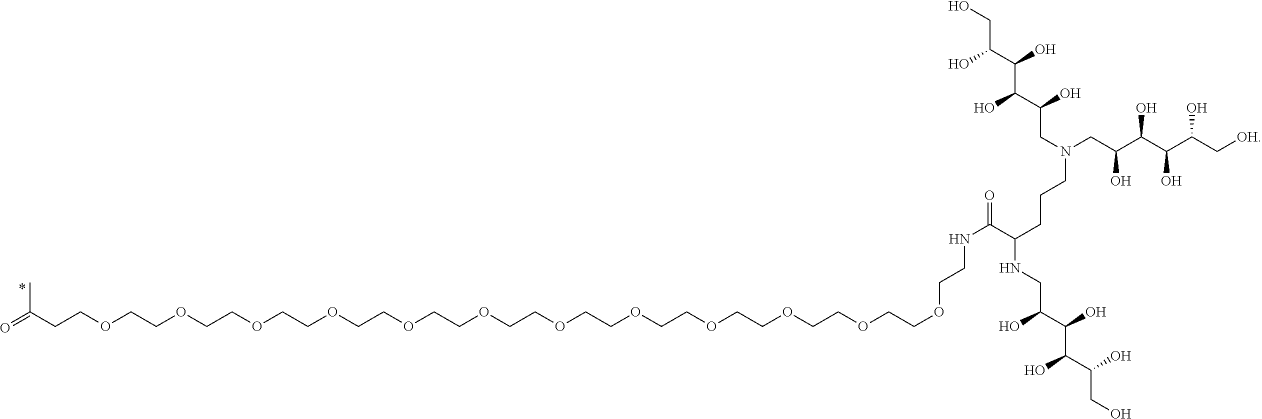 [SMILES: CC(=O)CCOCCOCCOCCOCCOCCOCCOCCOCCOCCOCCOCCOCCNC(=O)C(CCCN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]

In some embodiments, provided is a conjugate comprising a Targeting unit attached to any of the Drug-Linkers described herein. In some embodiments, provided is a conjugate, wherein the Targeting unit is selected from an antibody or an antigen-binding portion thereof. In some embodiments, provided is a conjugate, wherein the Targeting unit is a monoclonal antibody, a Fab, a Fab′, an F(ab′), an Fv, a disulfide linked Fc, a scFv, a single domain antibody, a diabody, a bi-specific antibody, or a multi-specific antibody. In some embodiments, provided is a conjugate, wherein the Targeting unit is a diabody, a DART, an anticalin, an affibody, an avimer, a DARPin, or an adnectin. In some embodiments, provided is a conjugate, wherein the Targeting unit is mono-specific. In some embodiments, provided is a conjugate, wherein the Targeting unit is bivalent. In some embodiments, provided is a conjugate, wherein the Targeting unit is bispecific. In some embodiments, provided is a conjugate, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

In some embodiments, provided is a conjugate, selected from the following:

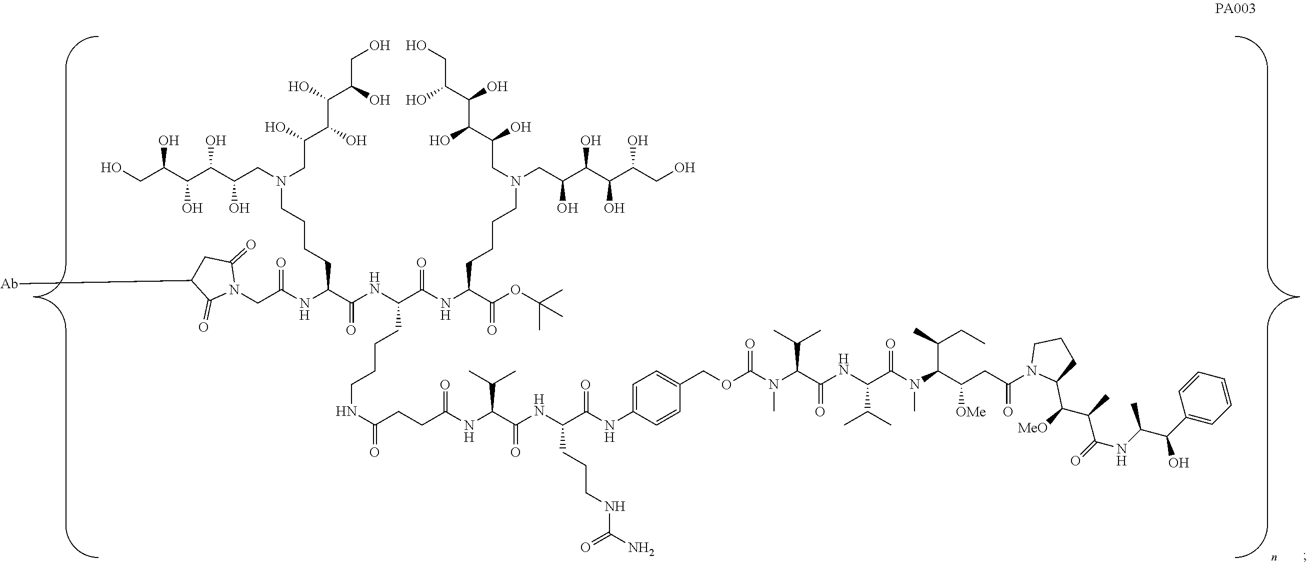

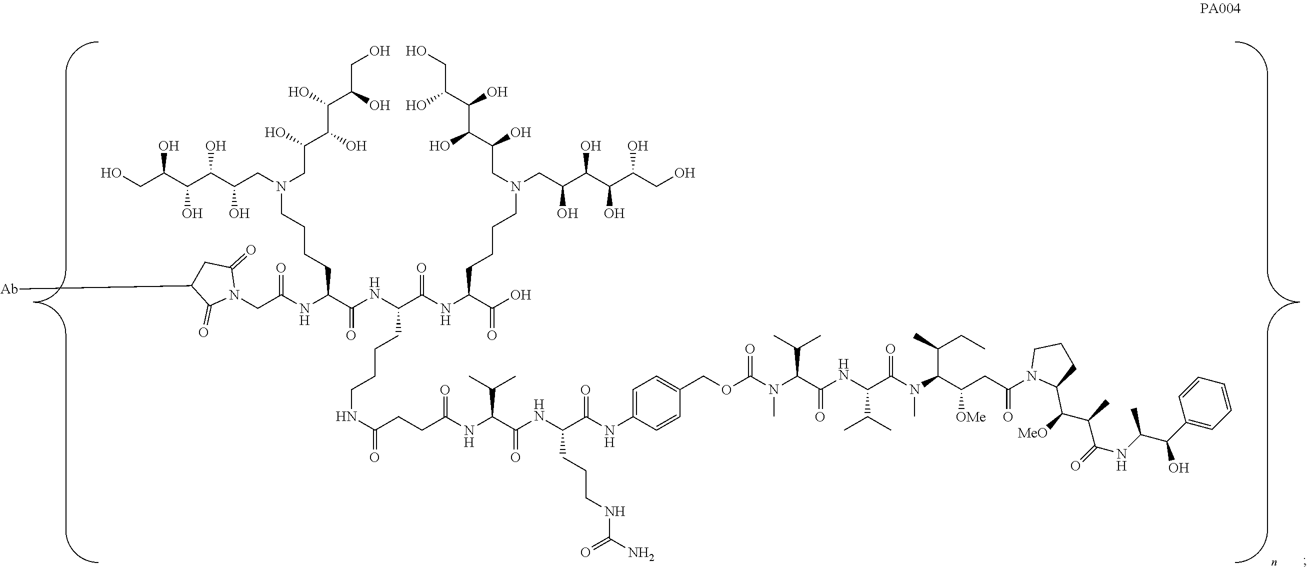

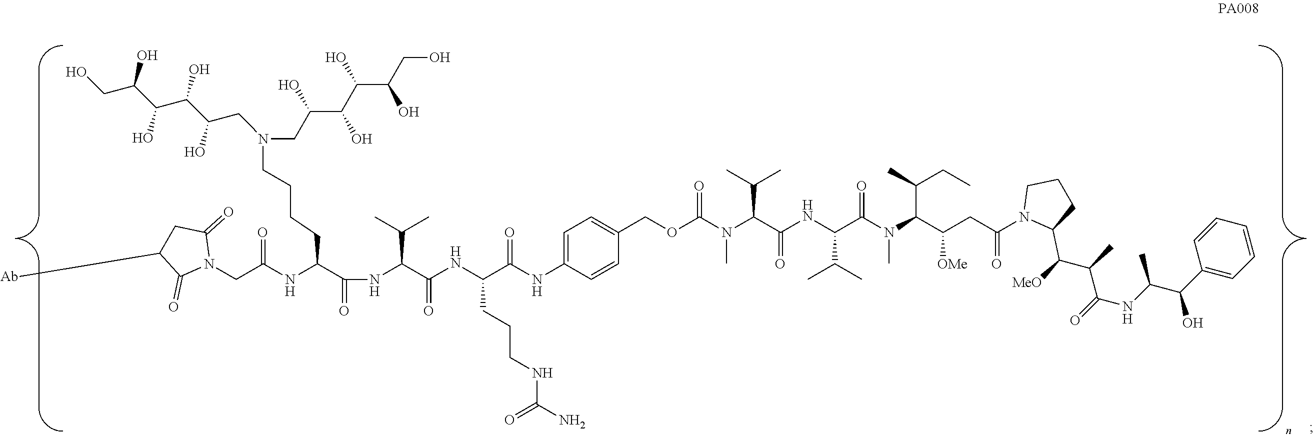

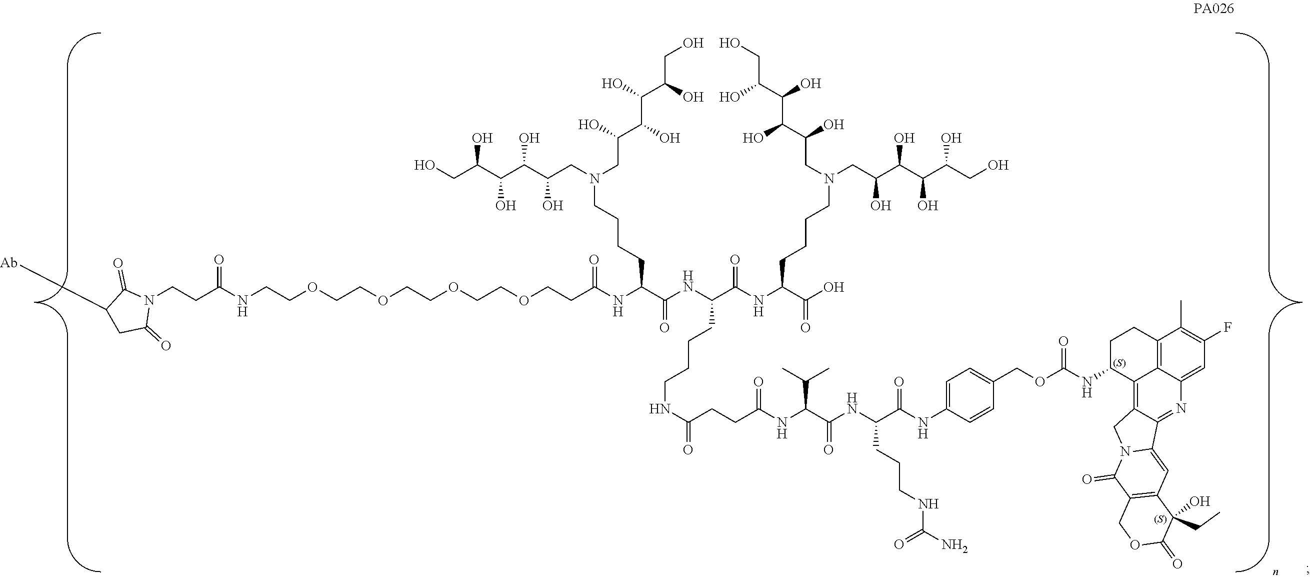

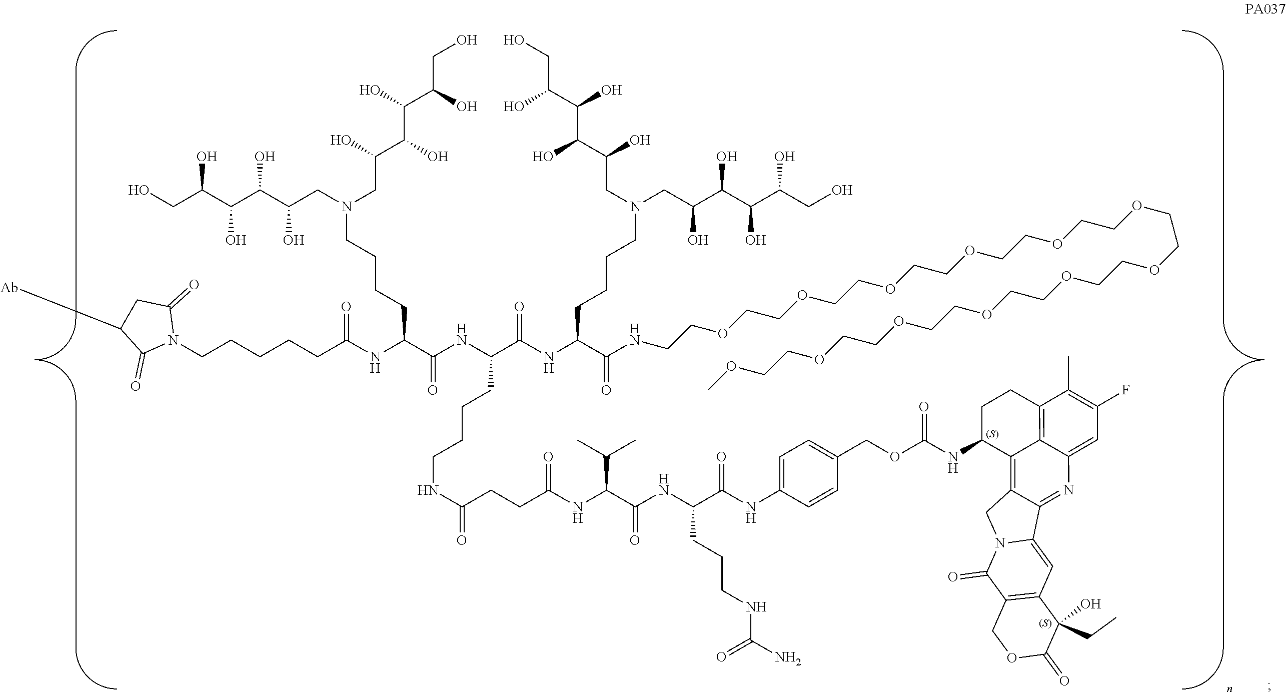

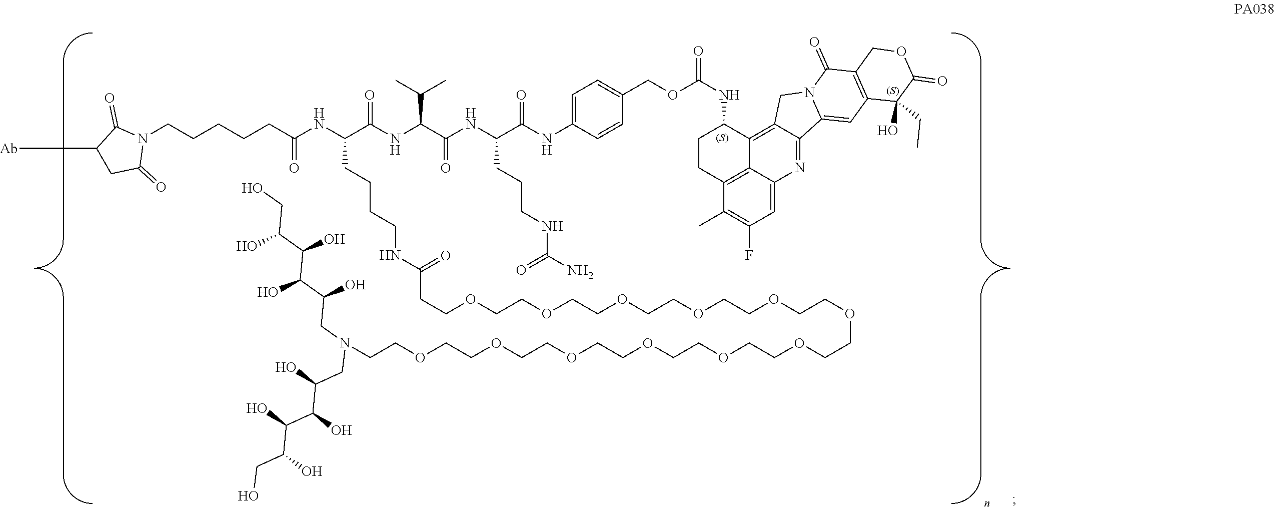

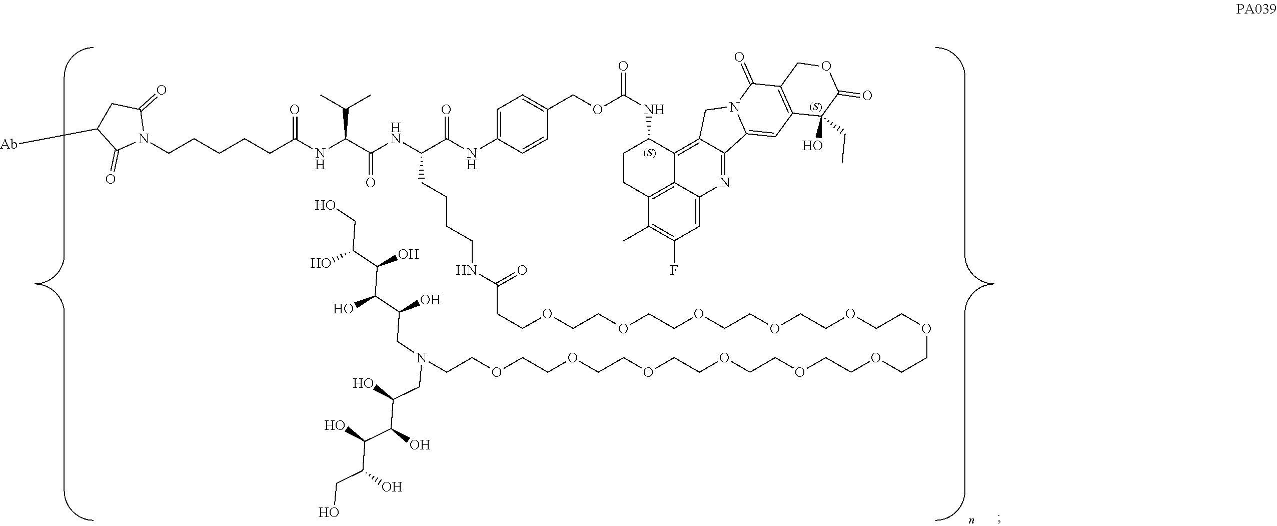

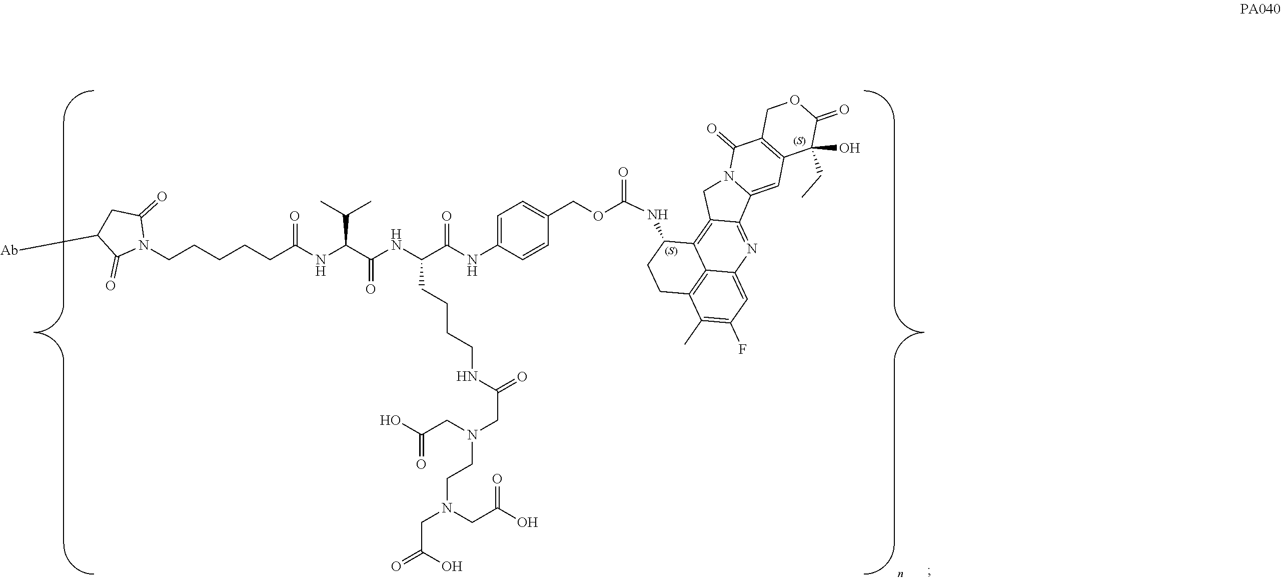

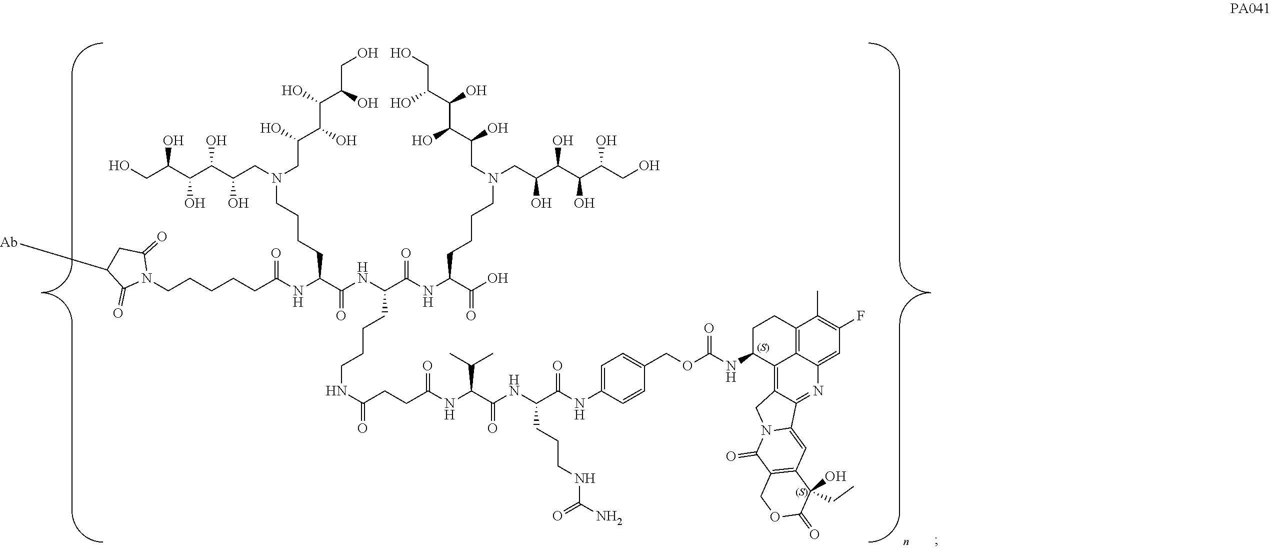

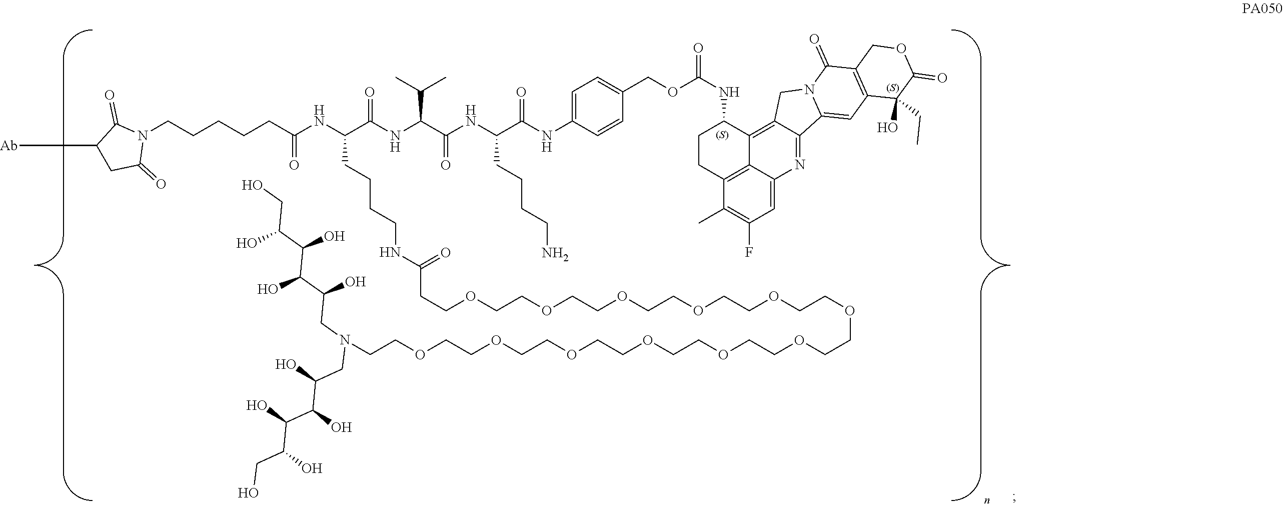

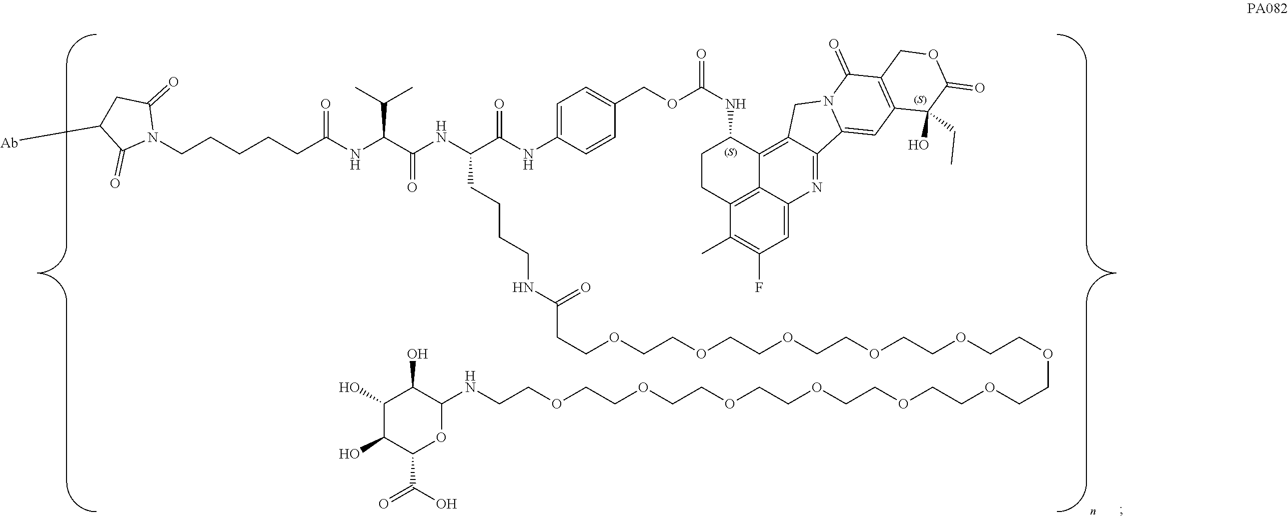

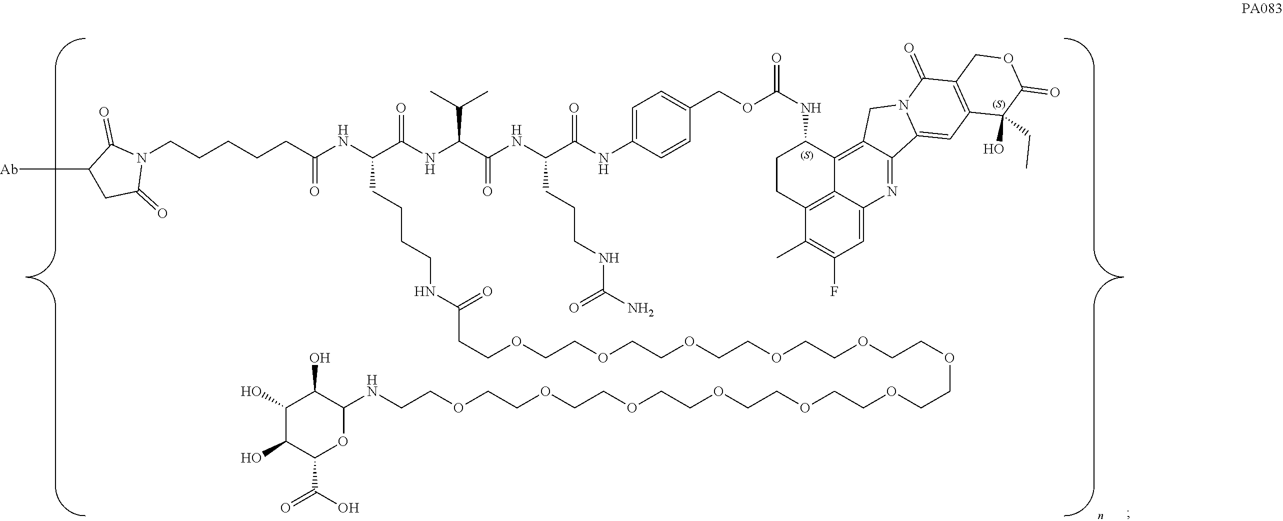

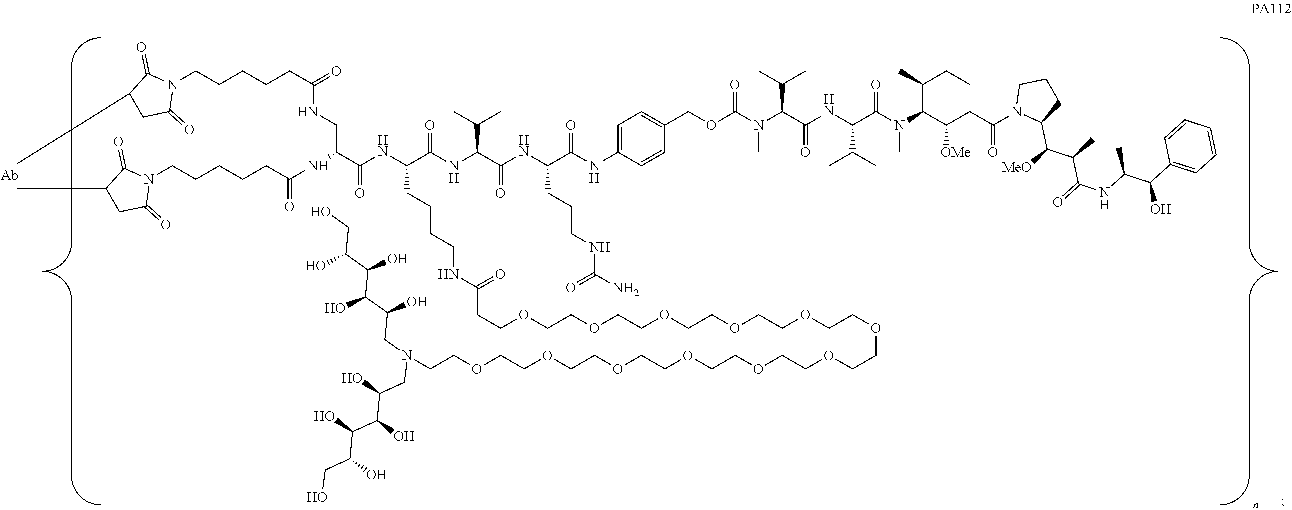

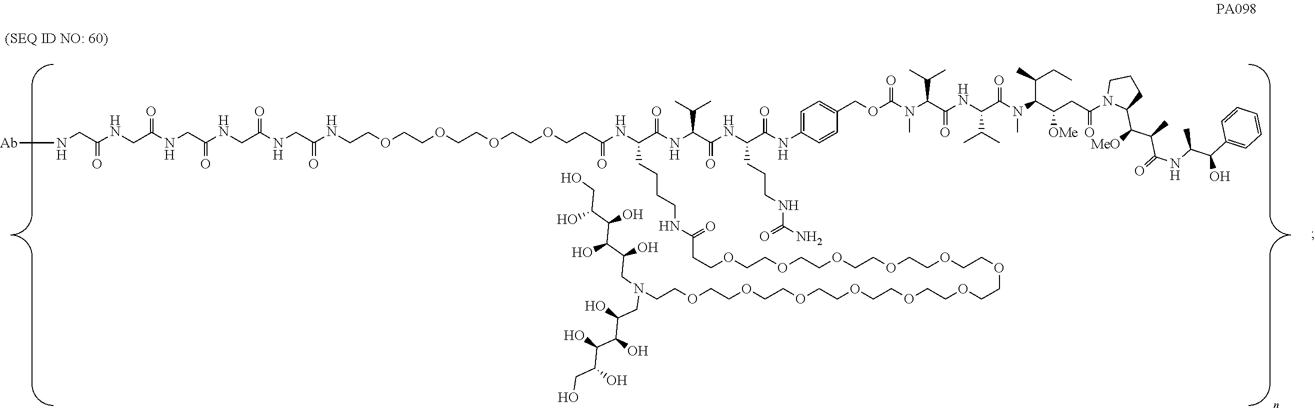

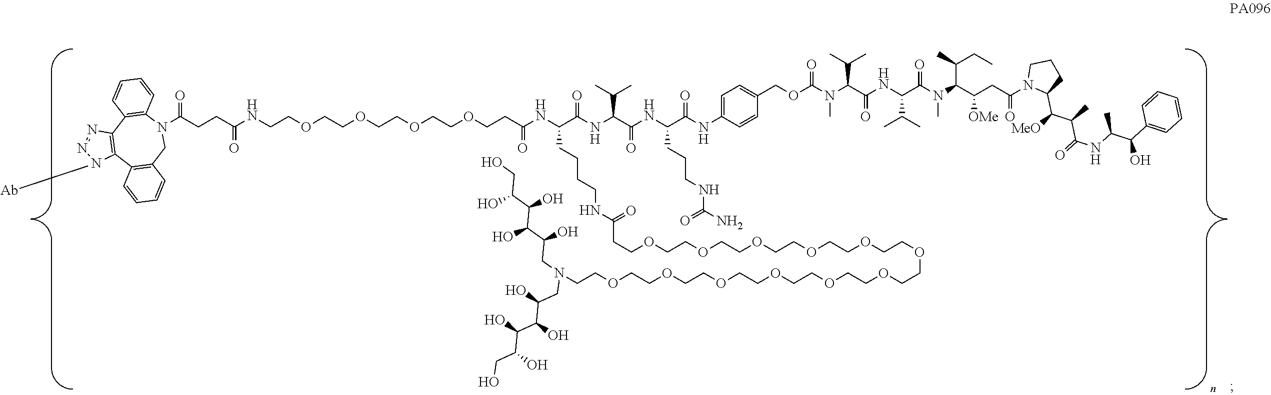

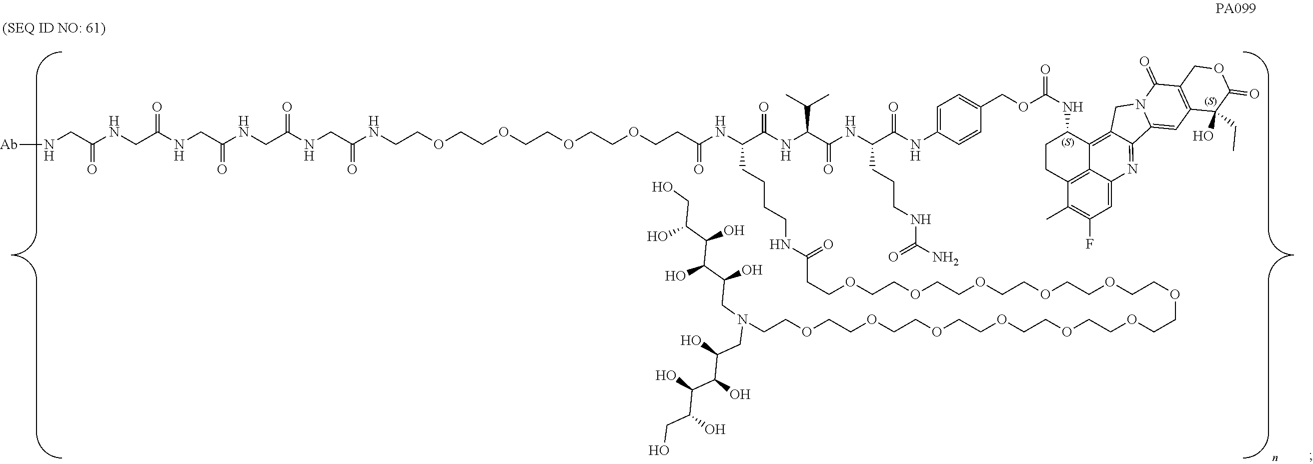

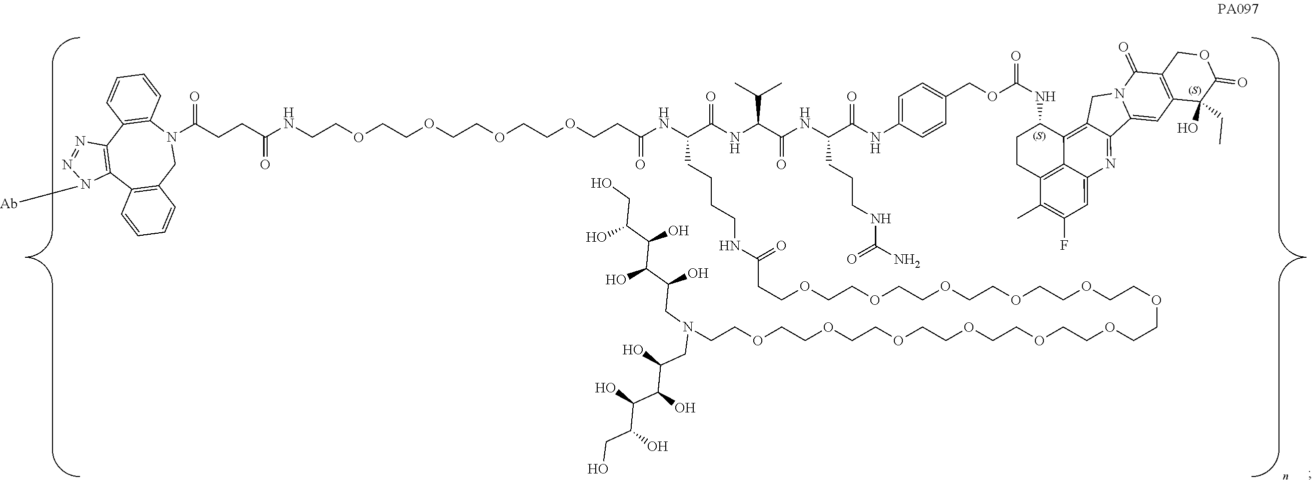

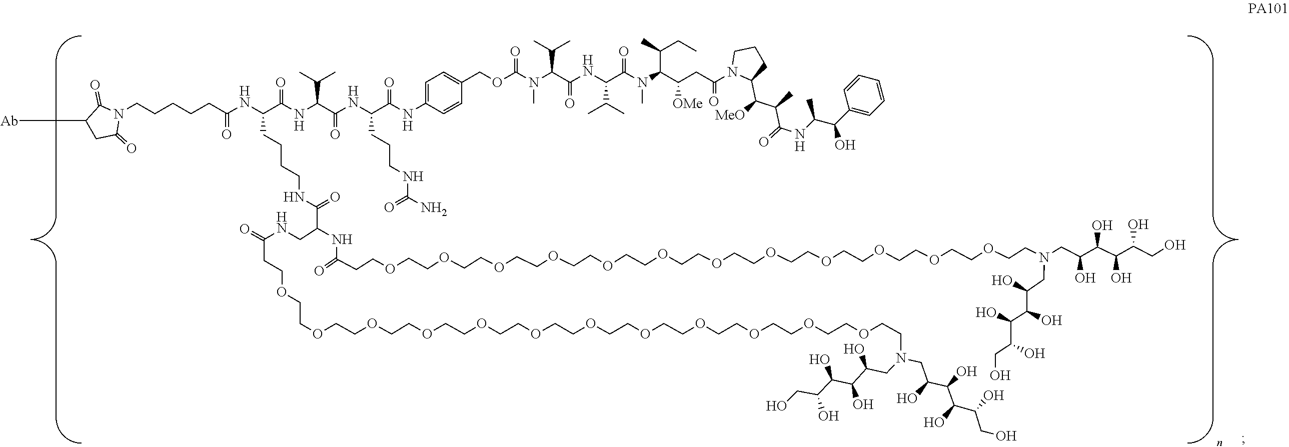

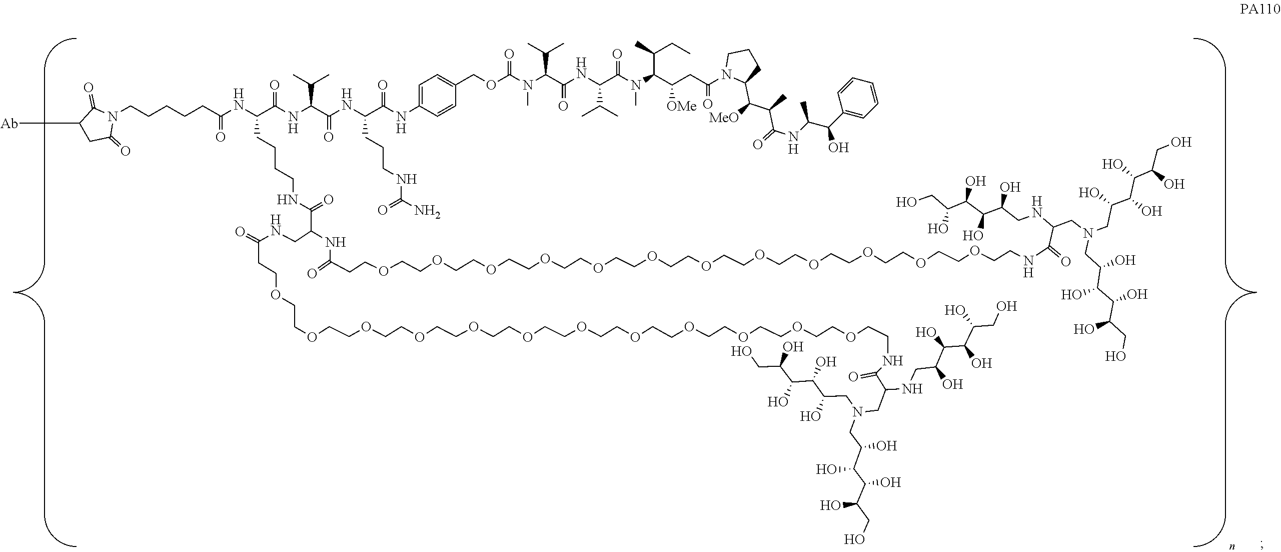

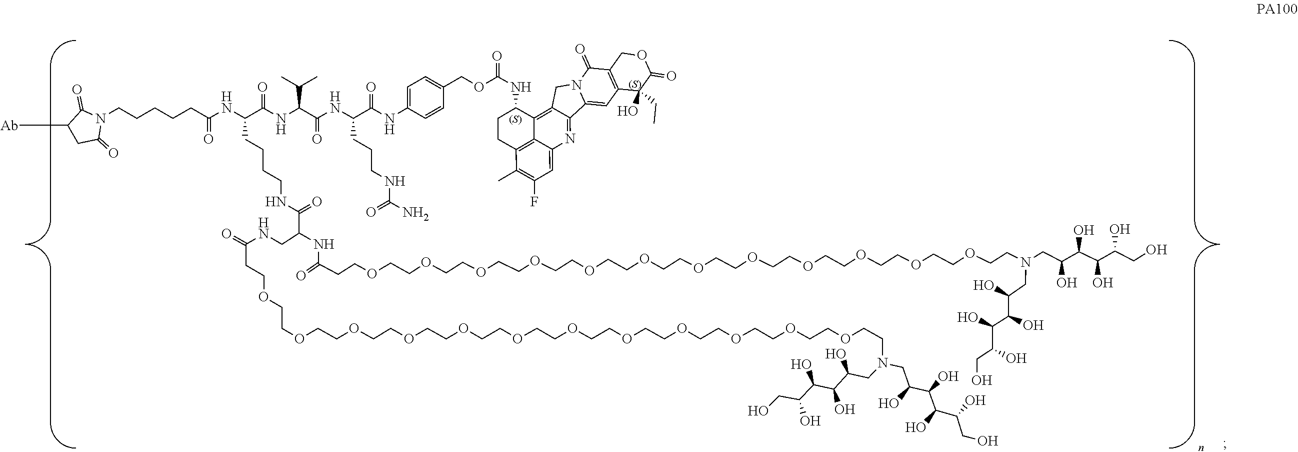

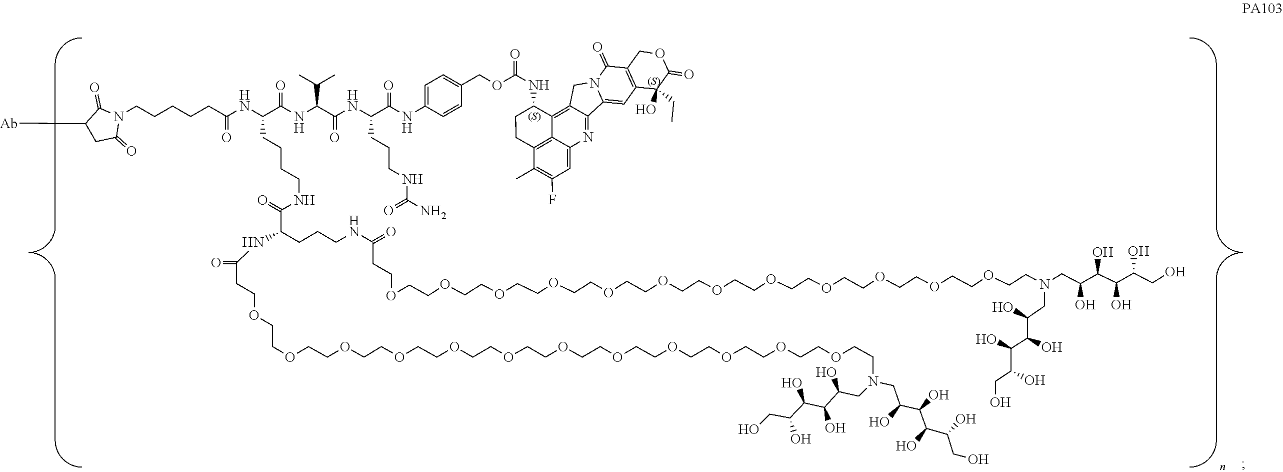

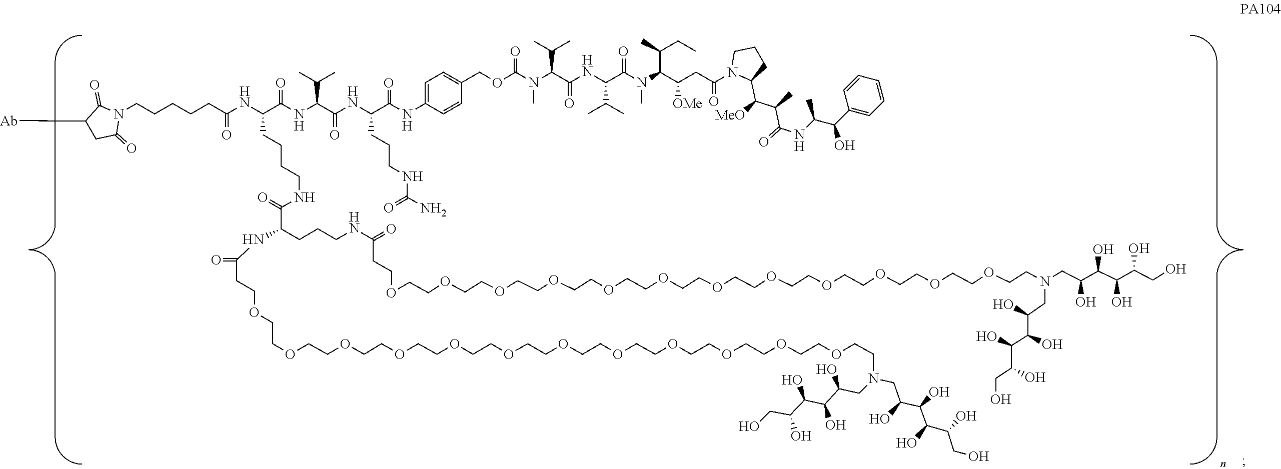

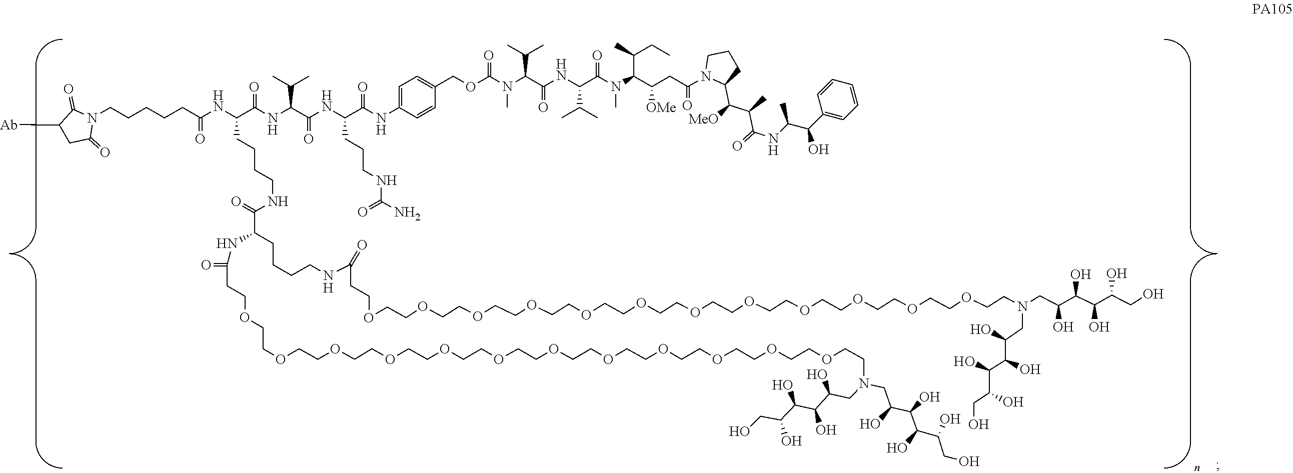

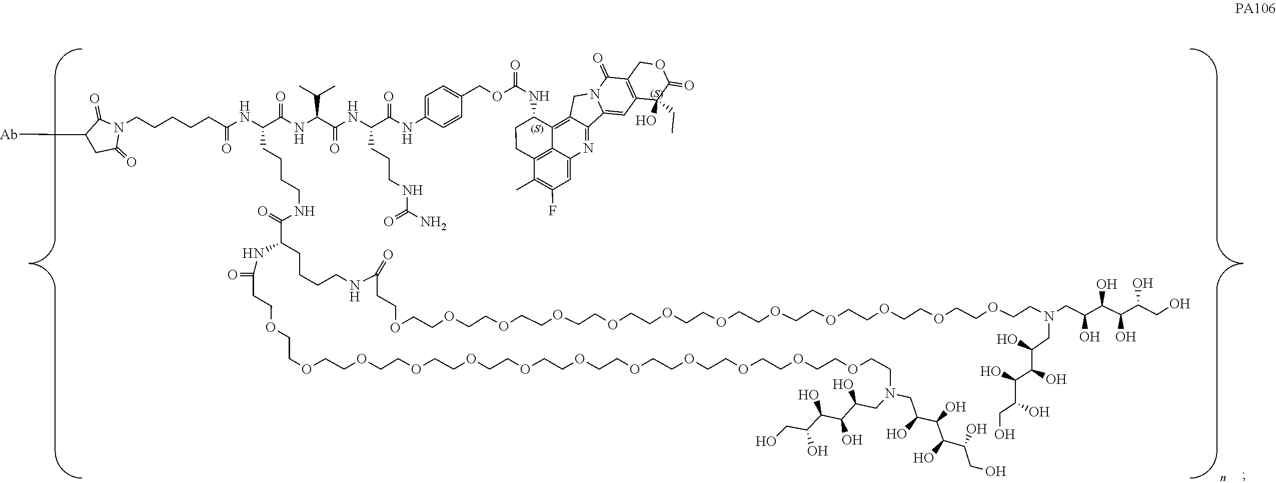

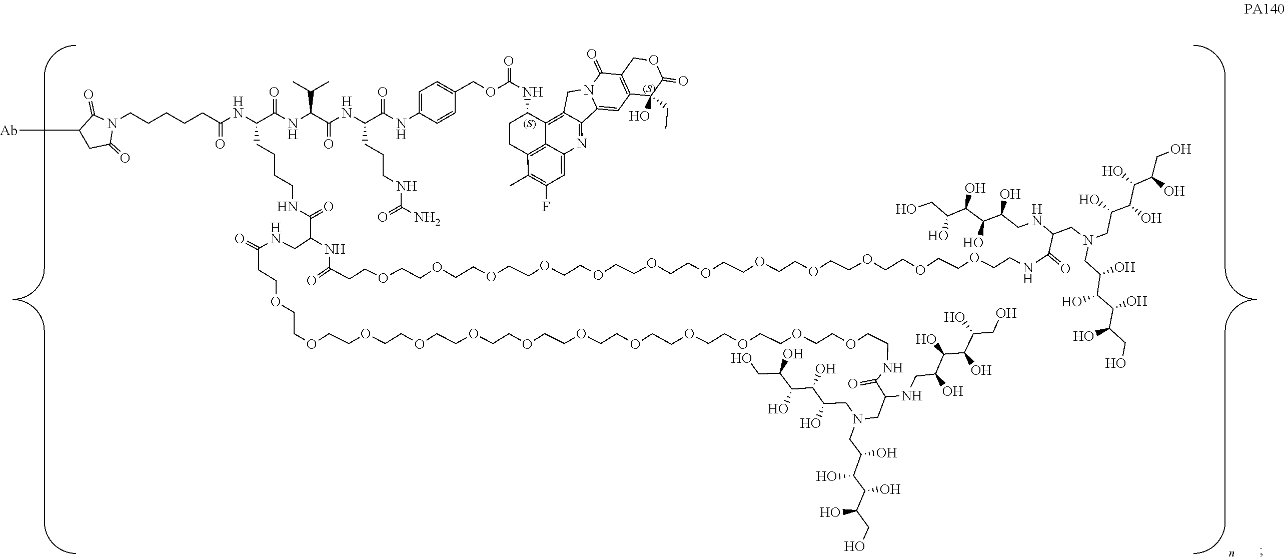

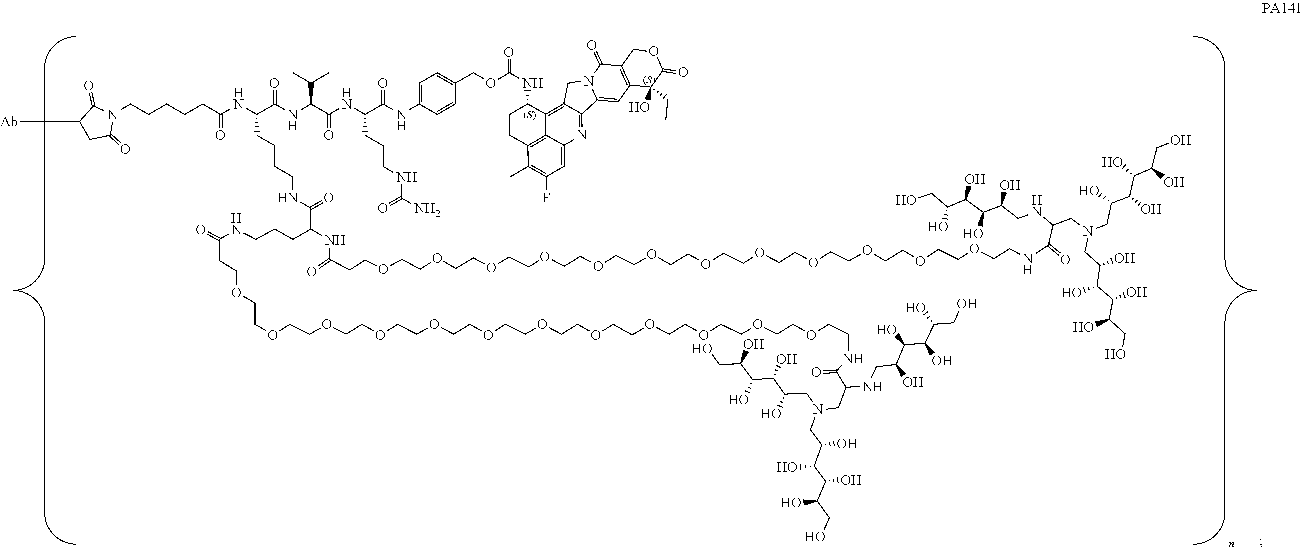

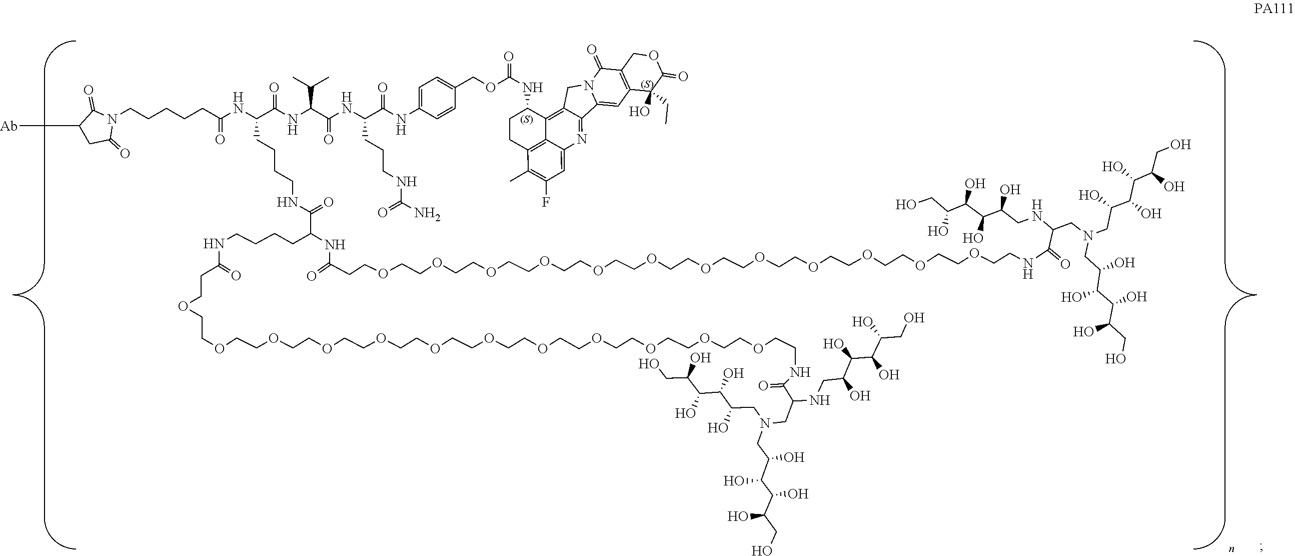

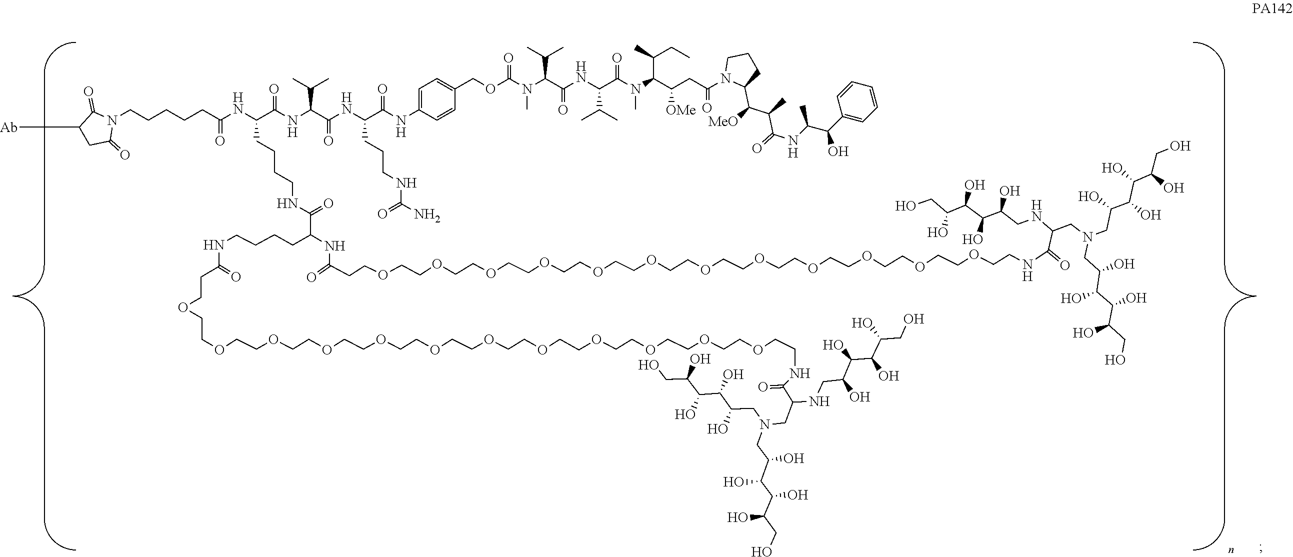

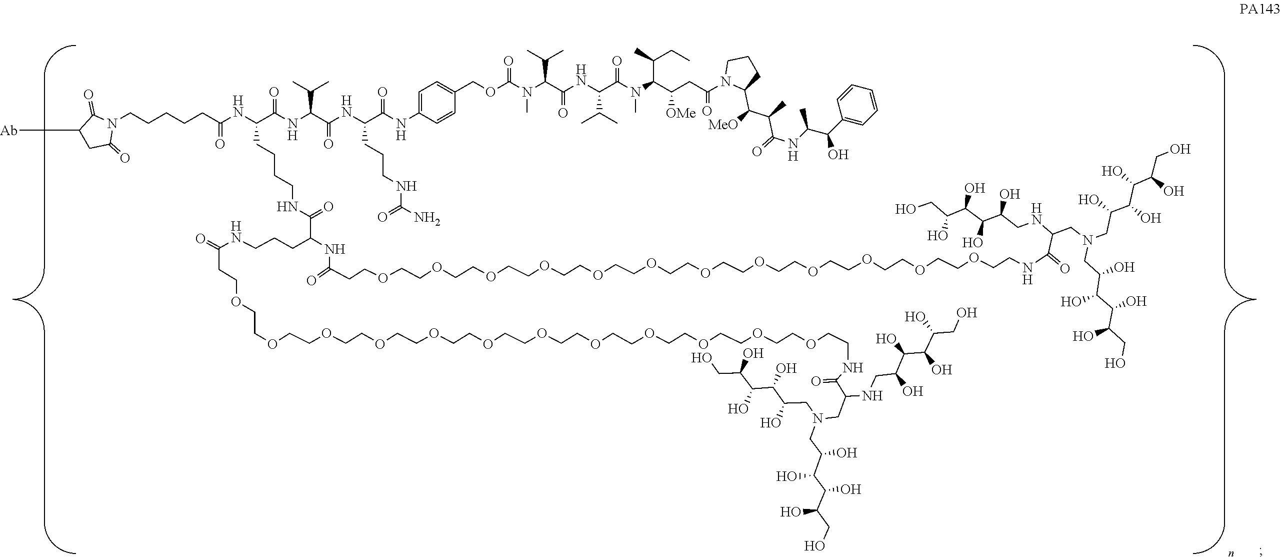

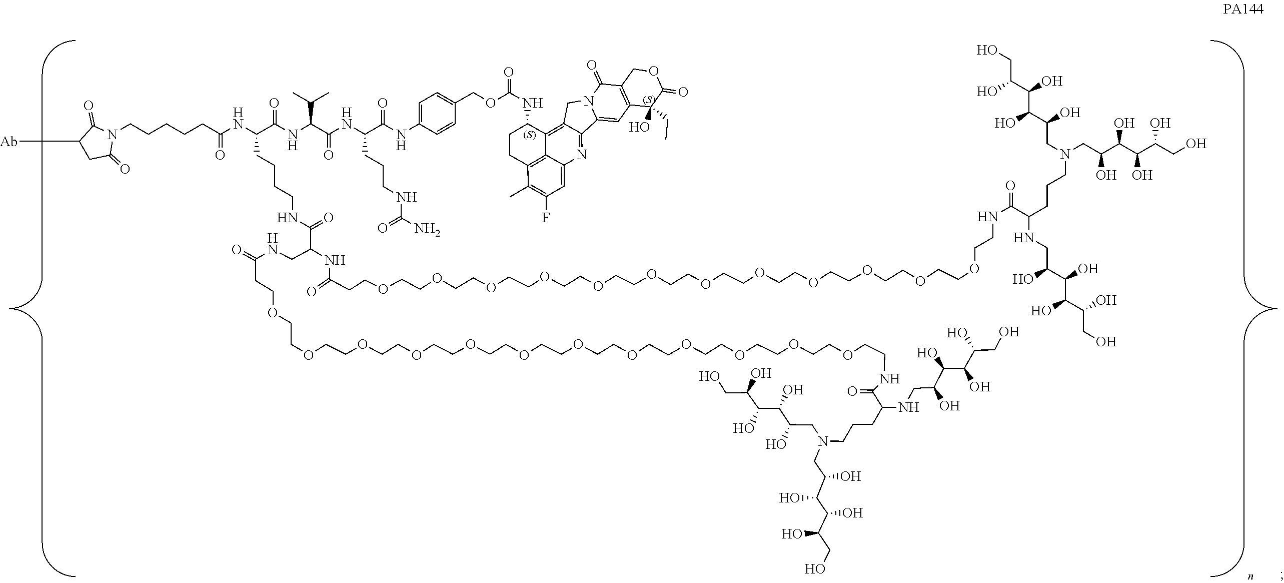

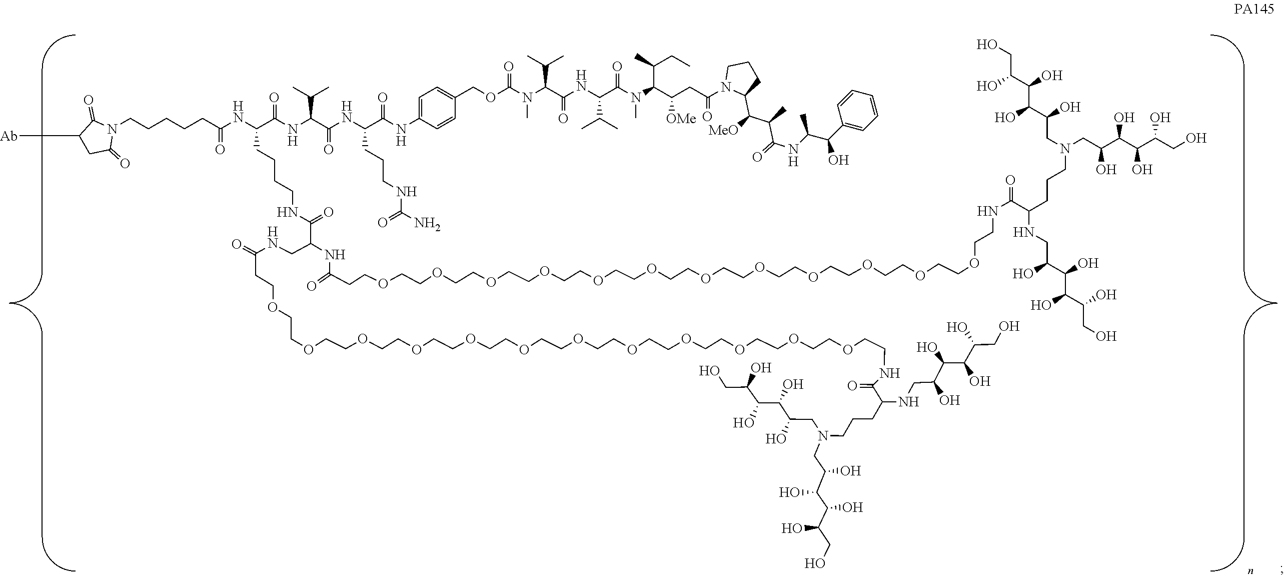

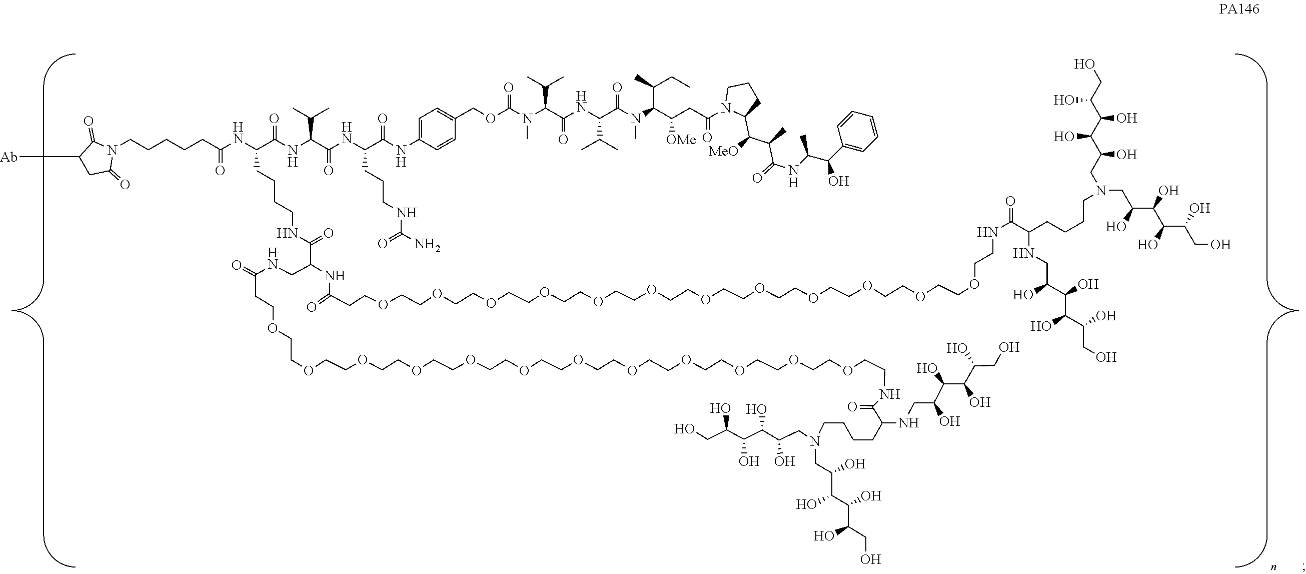

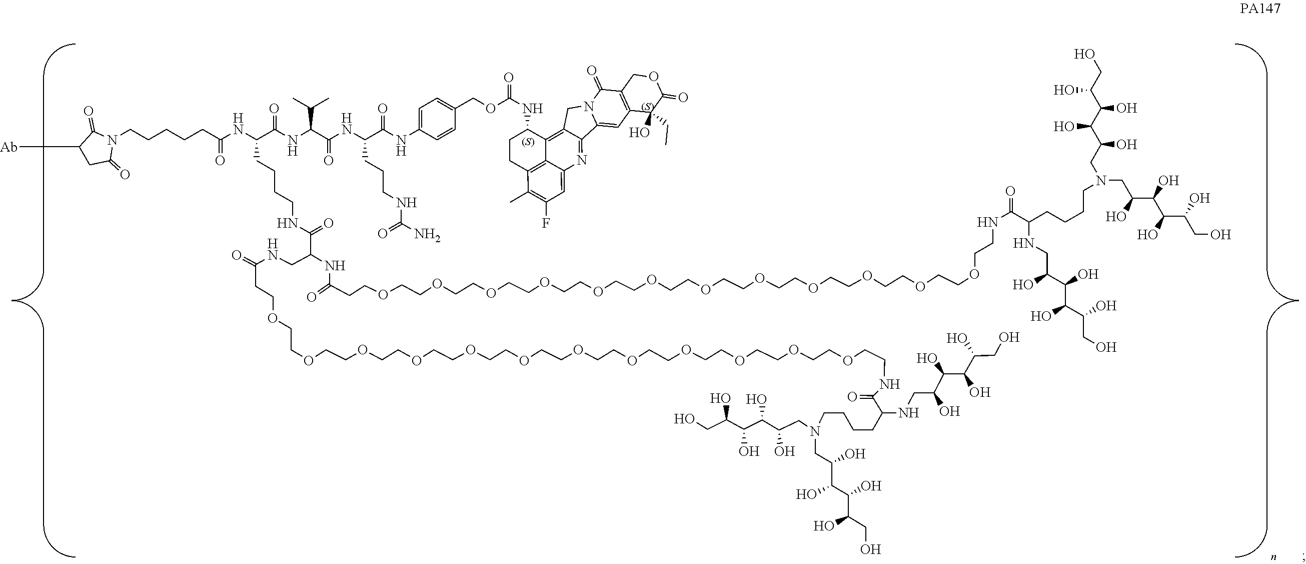

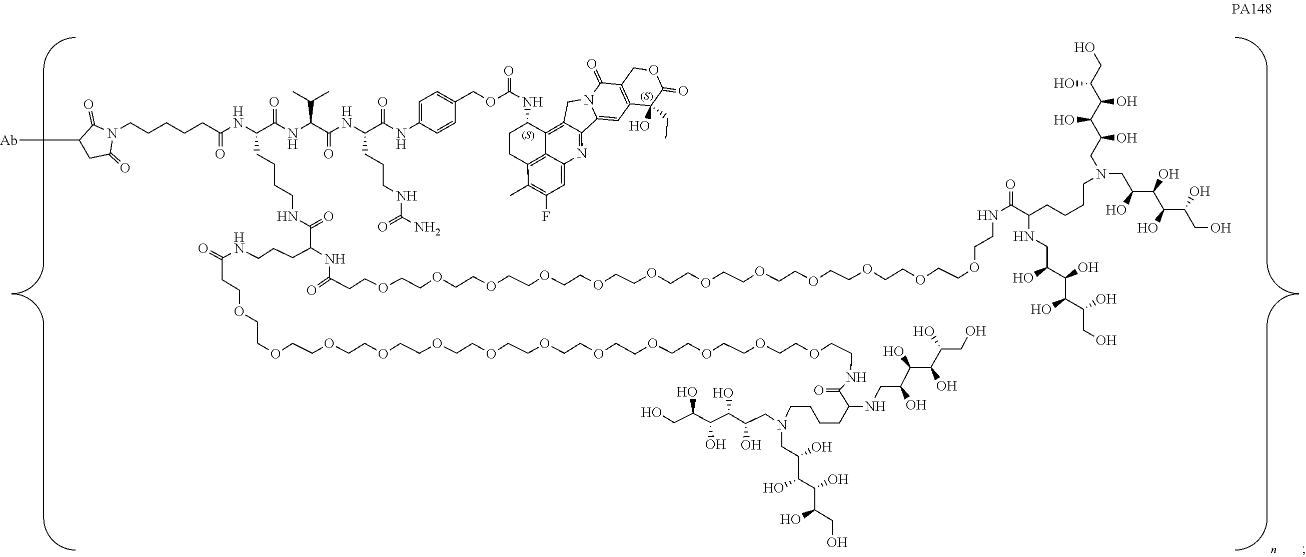

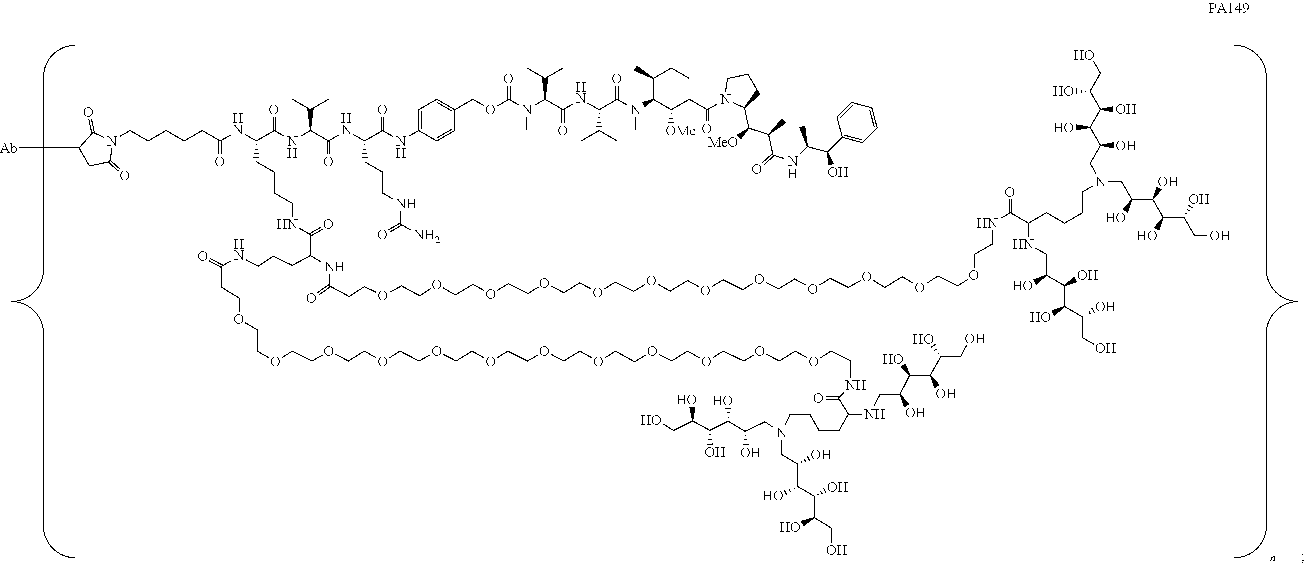

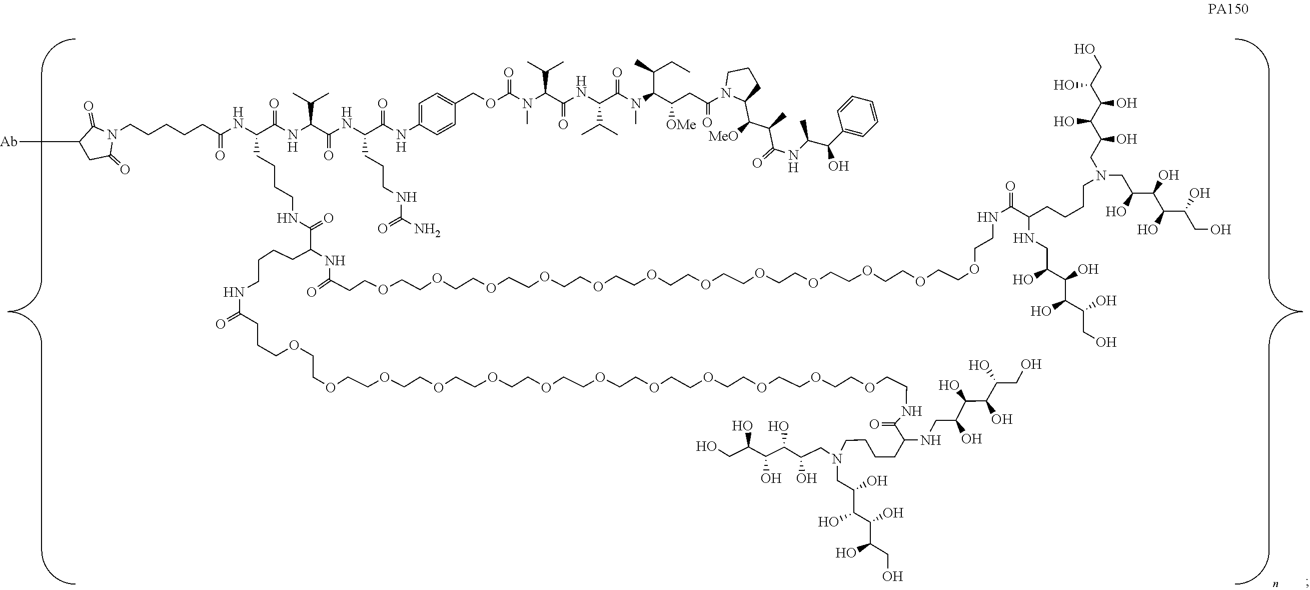

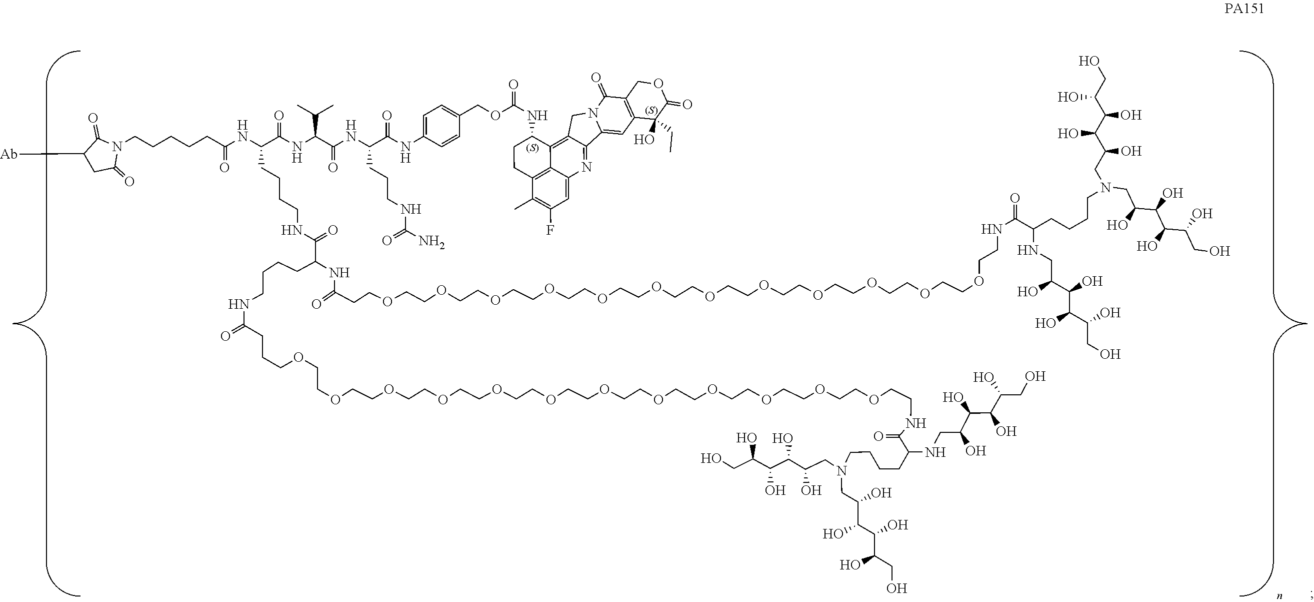

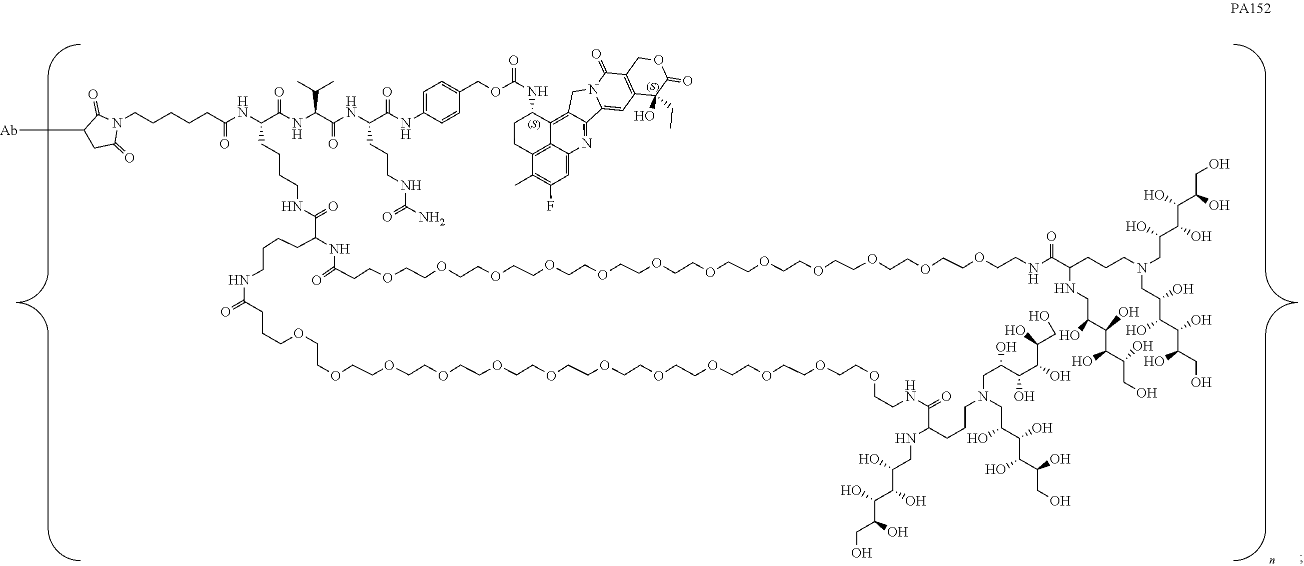

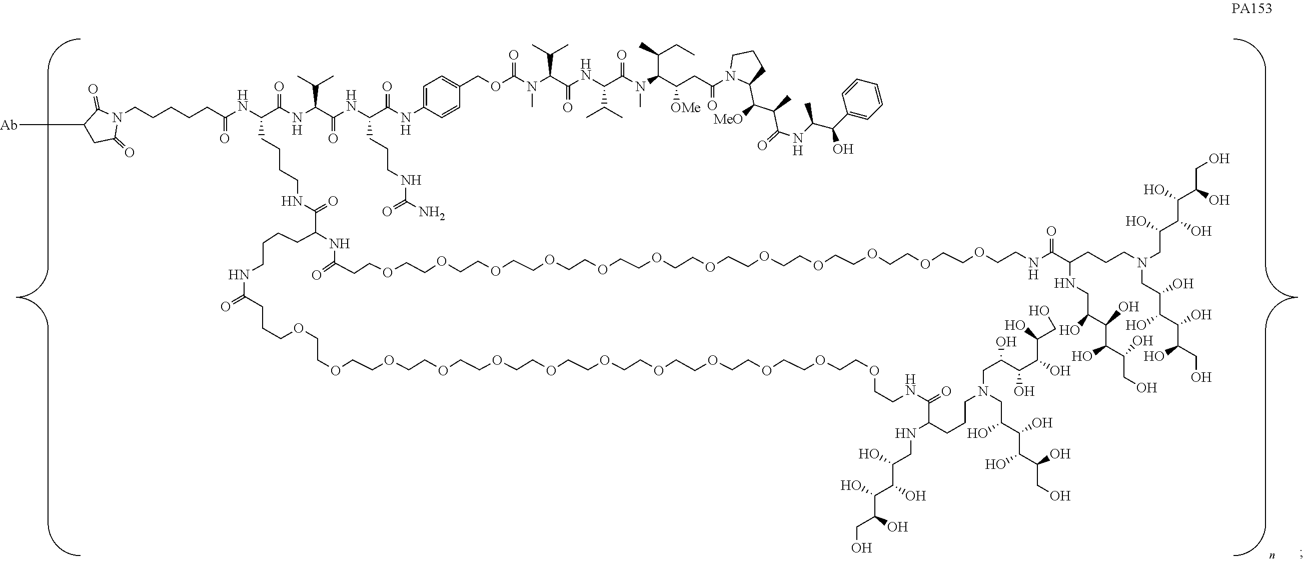

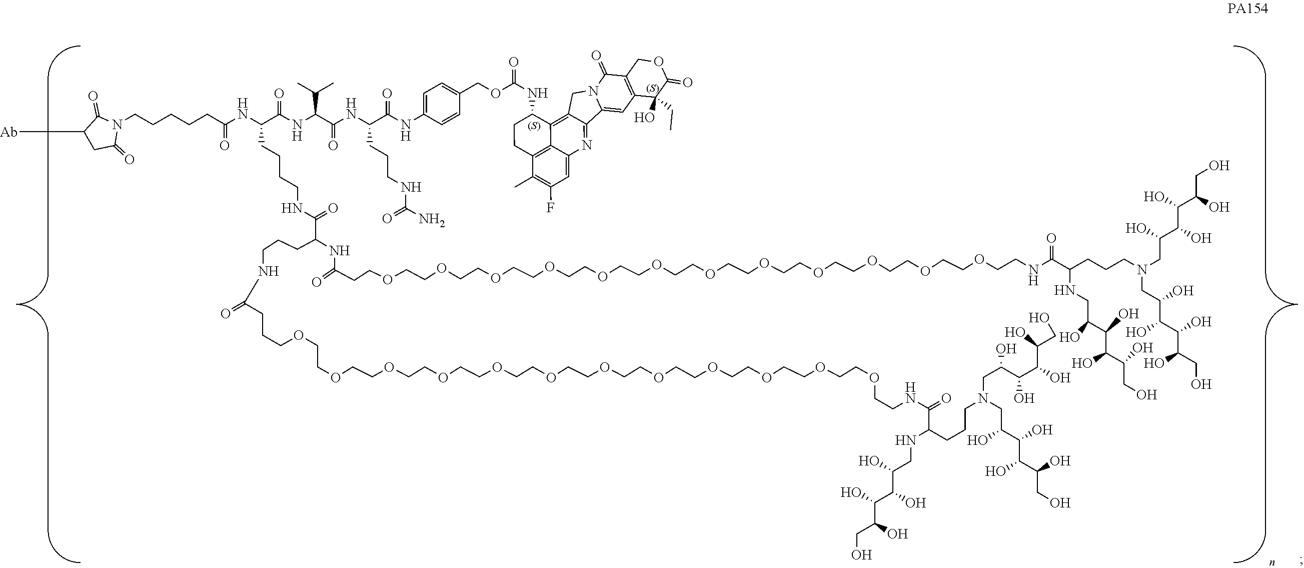

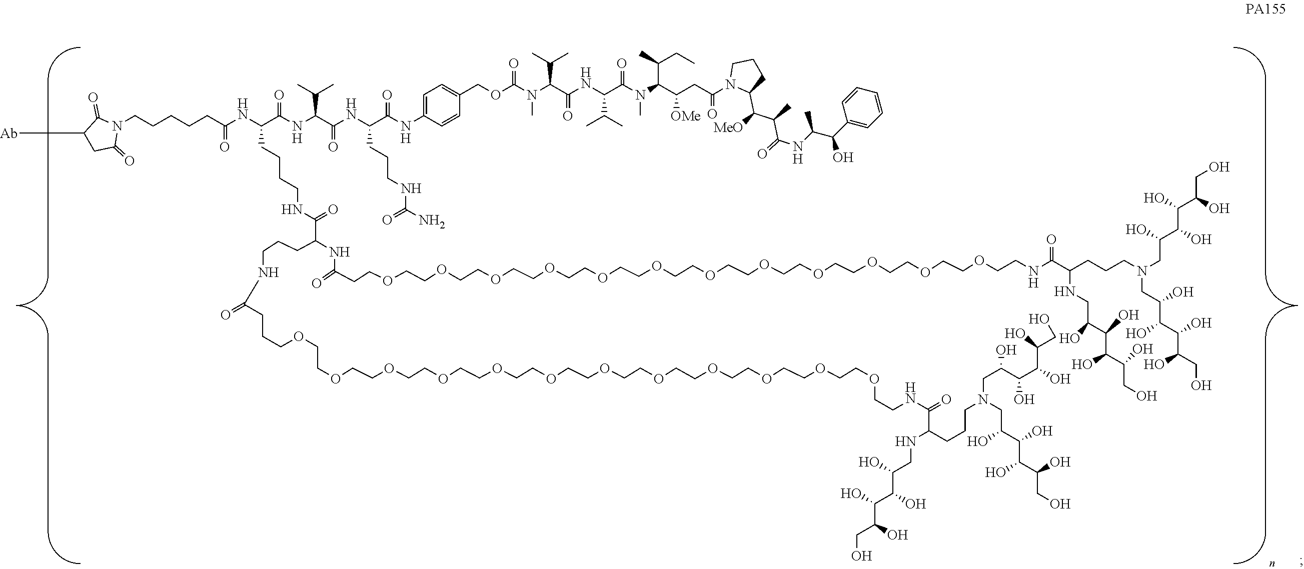

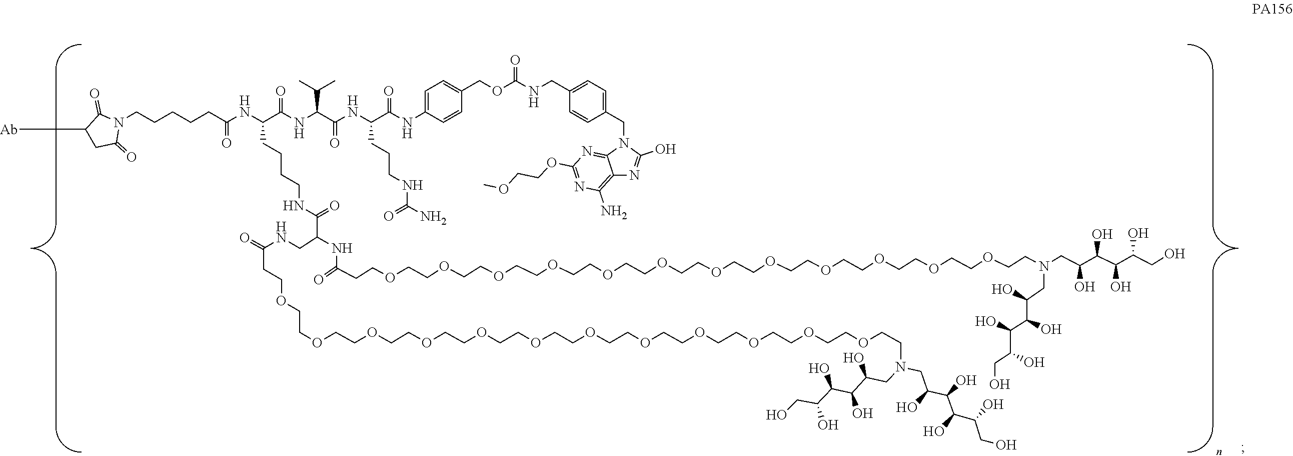

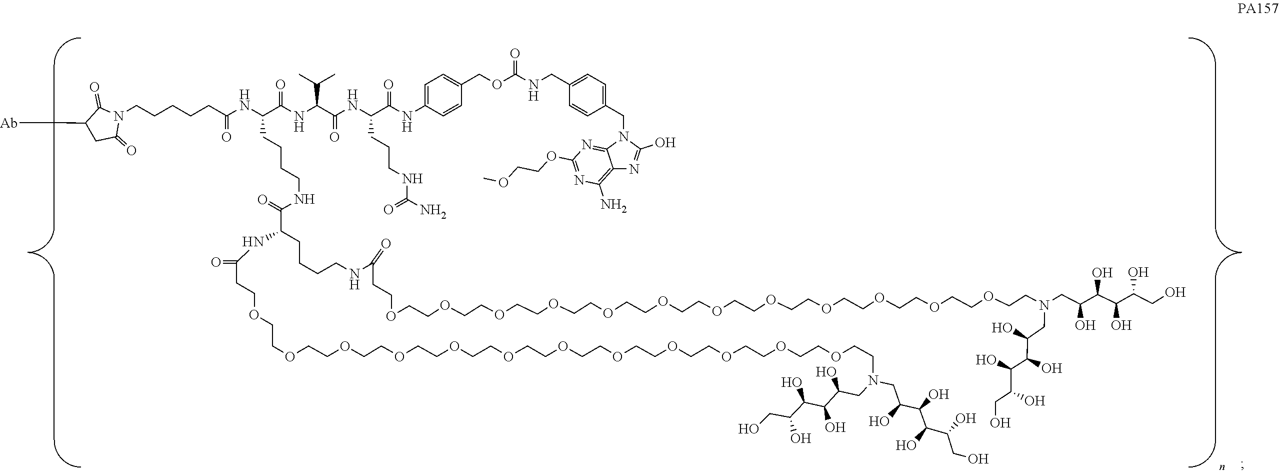

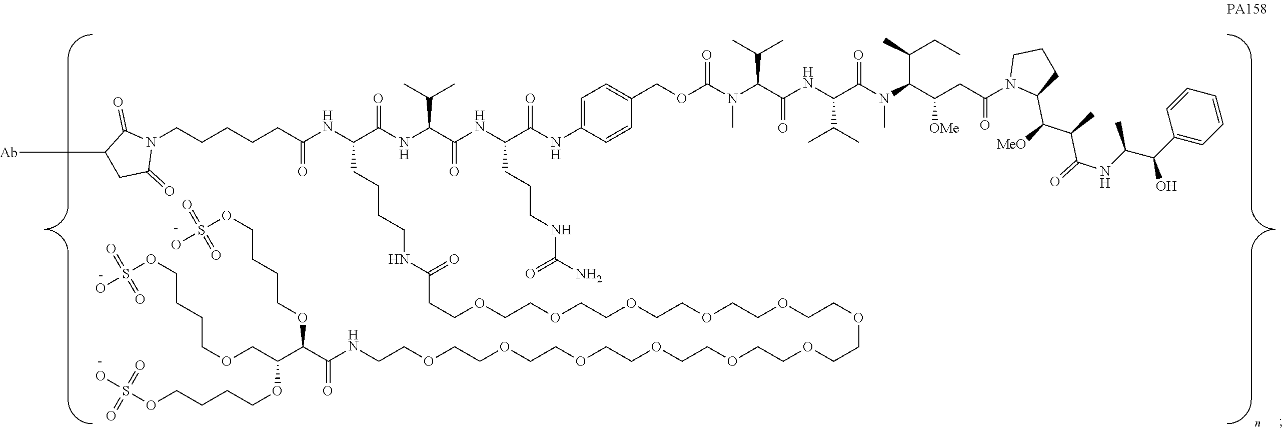

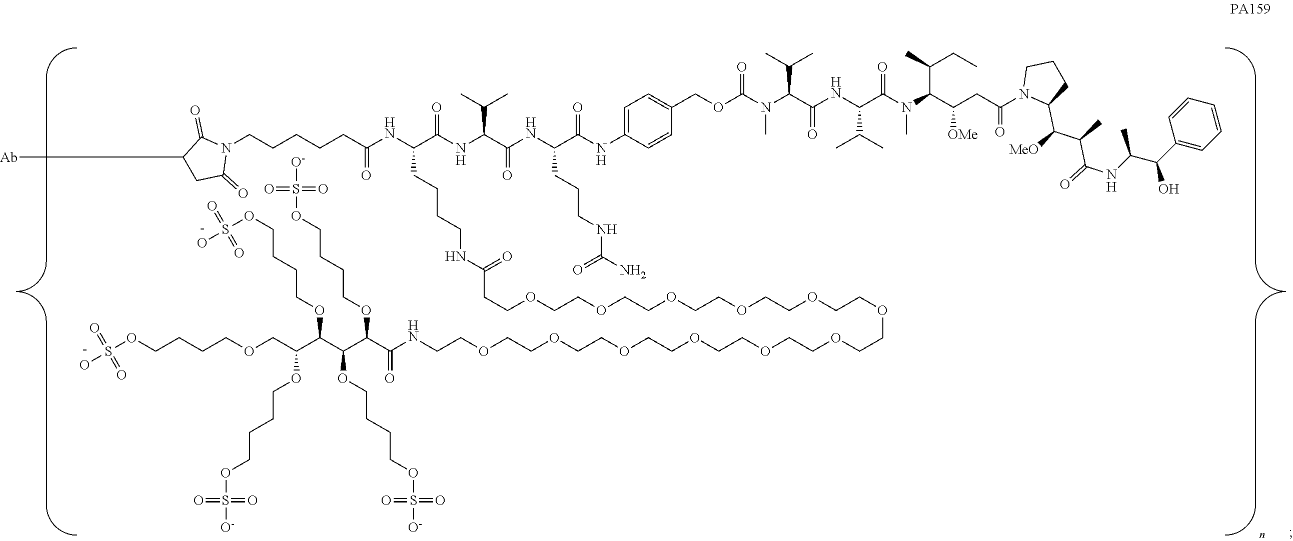

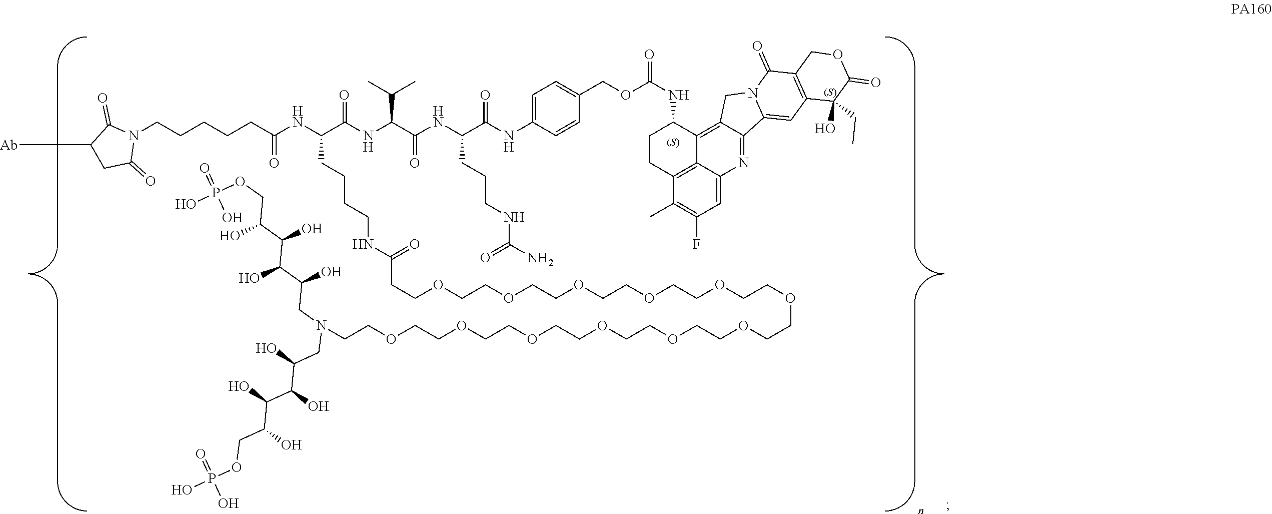

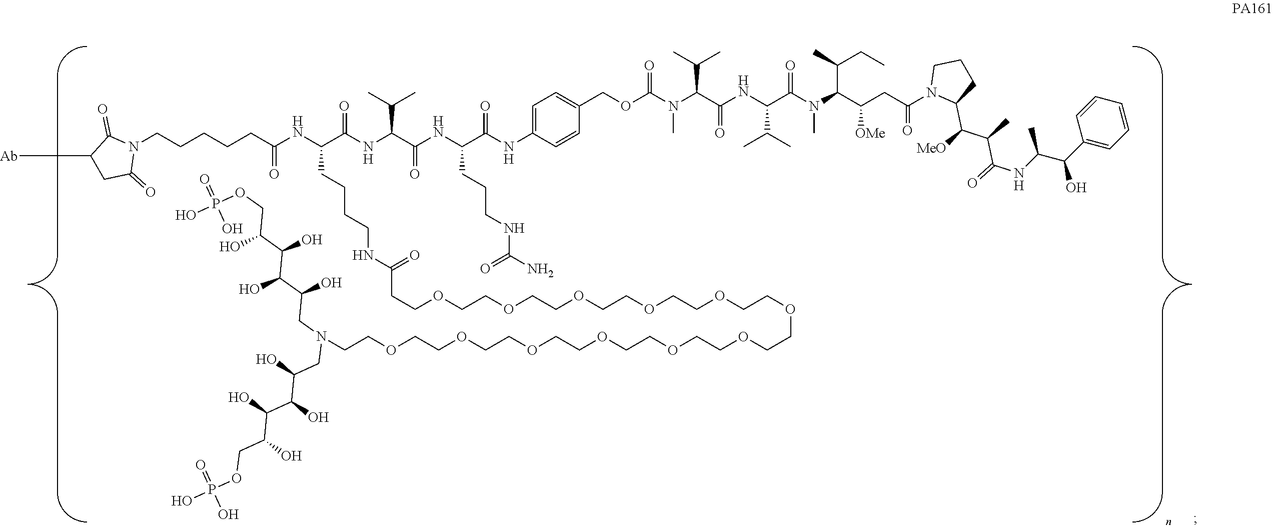

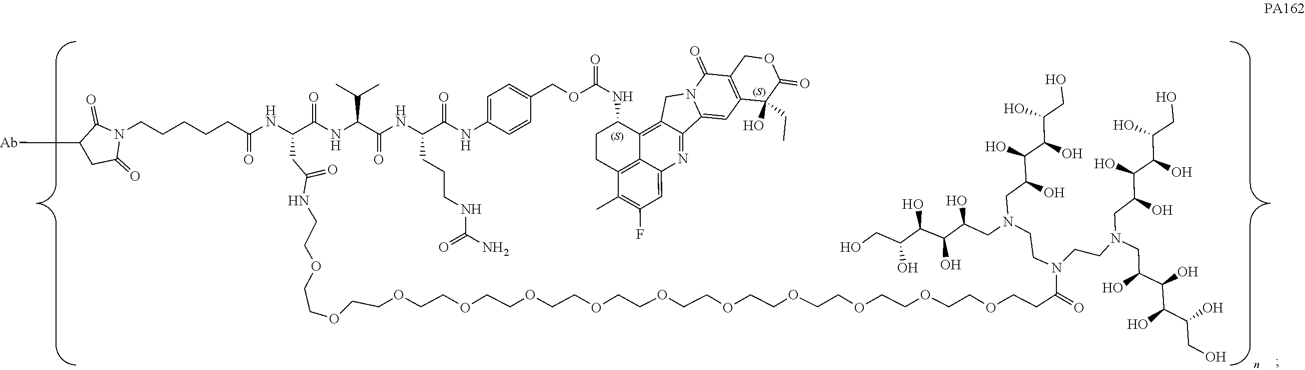

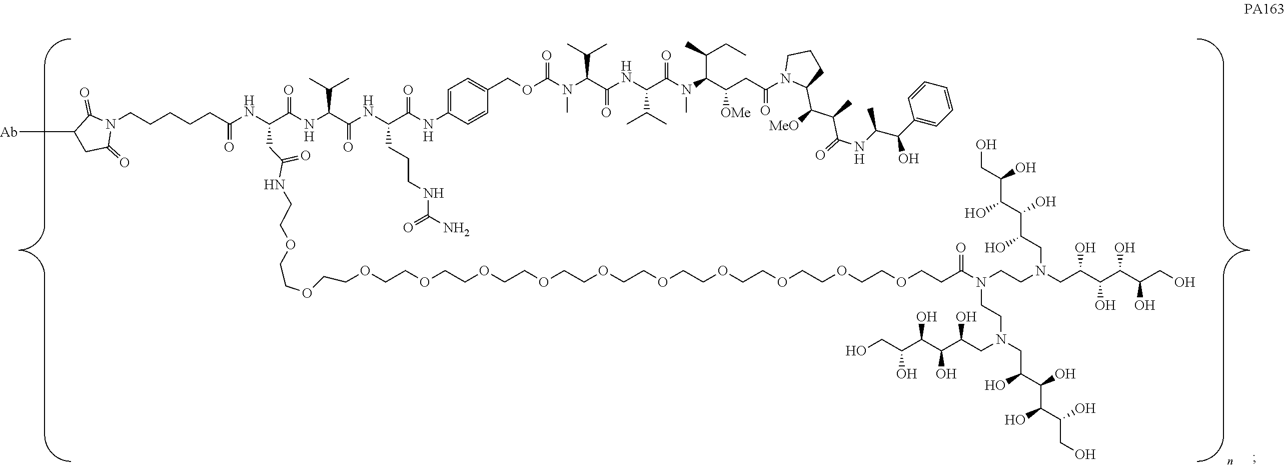

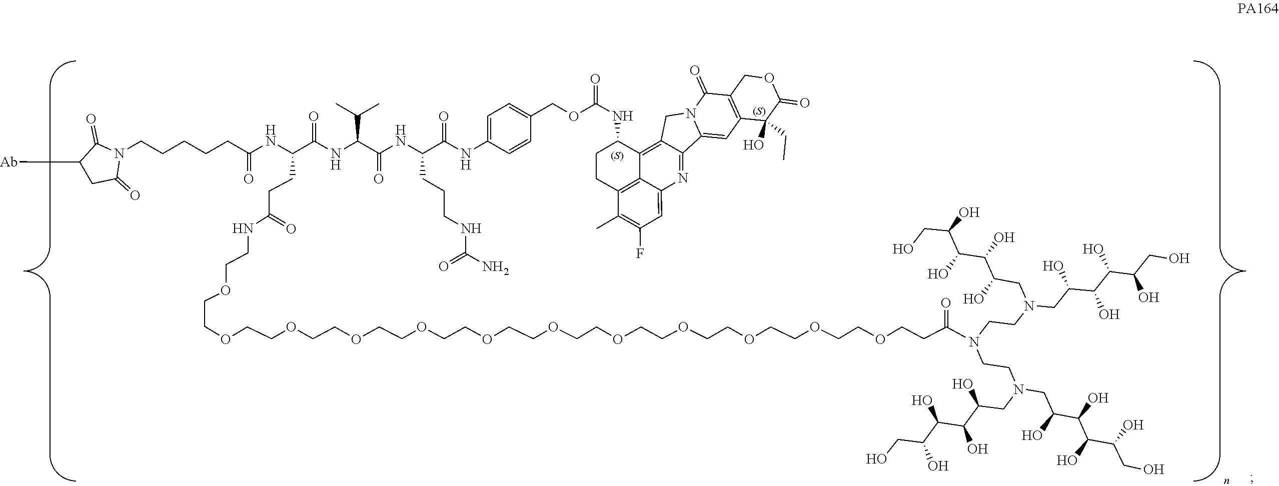

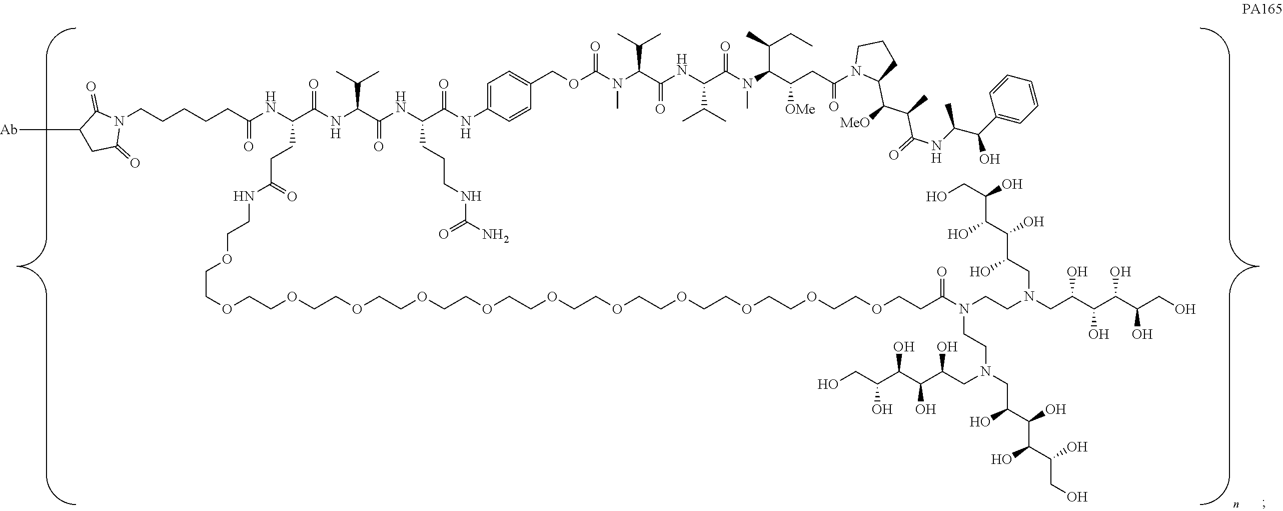

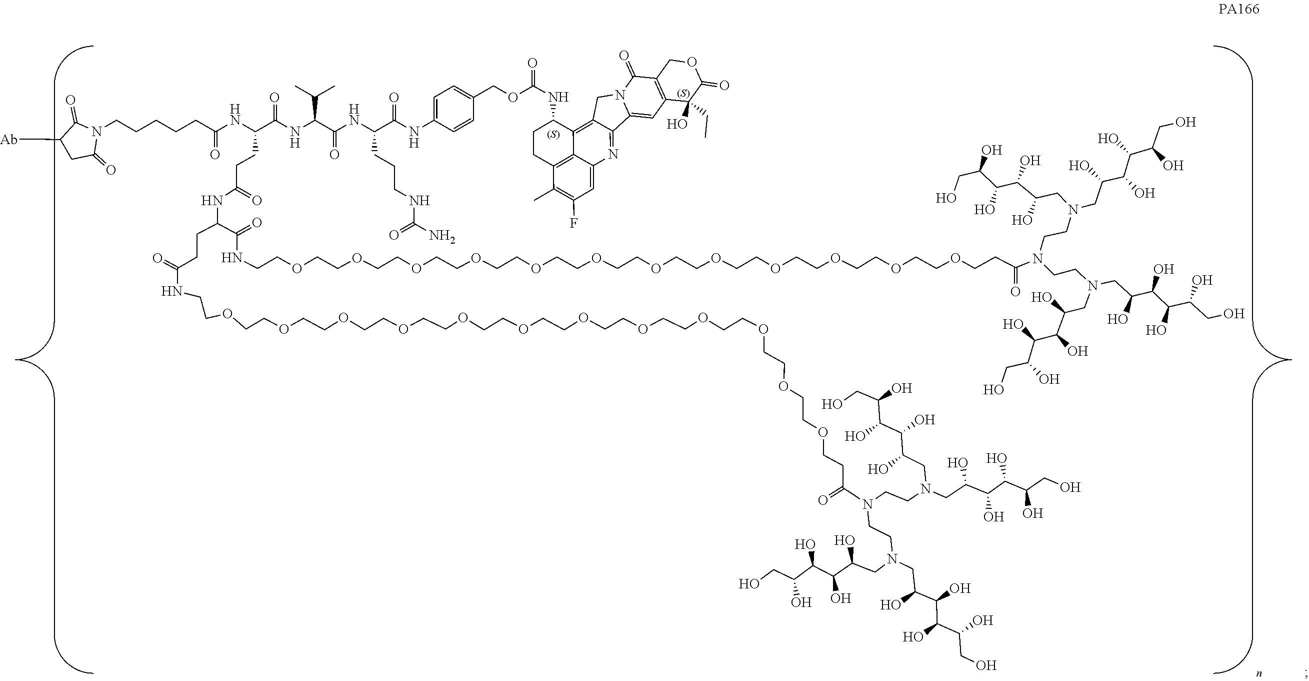

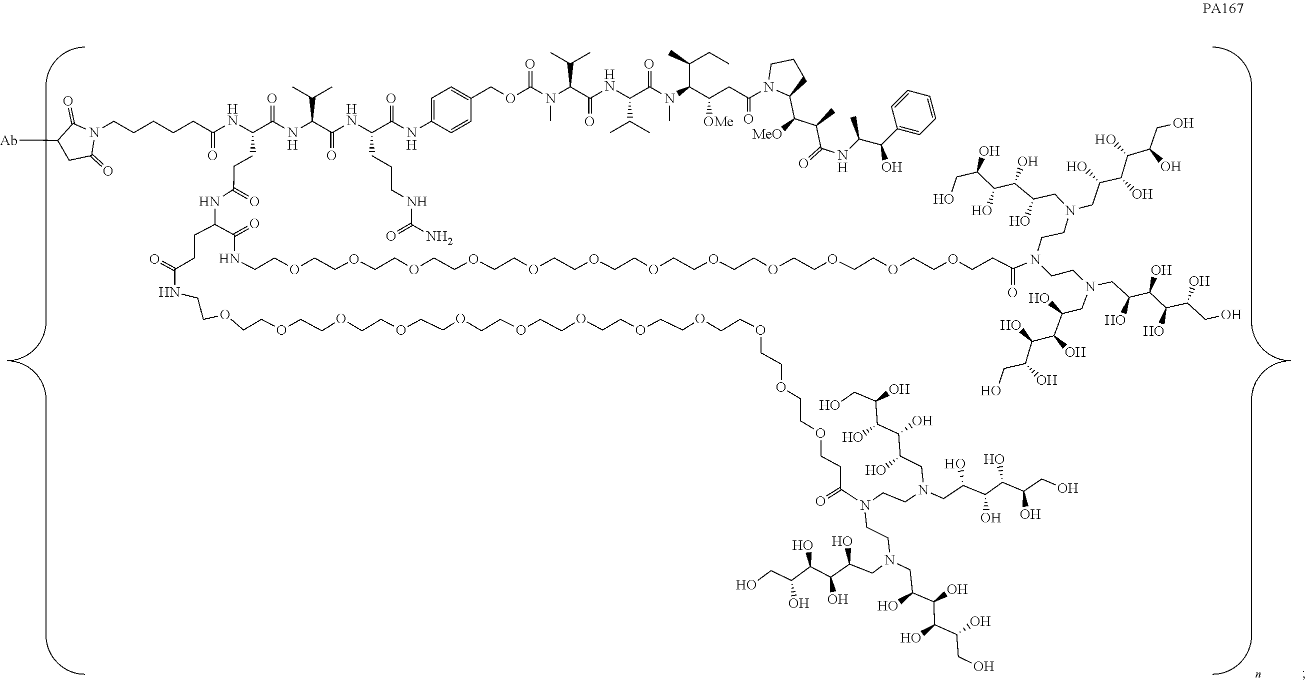

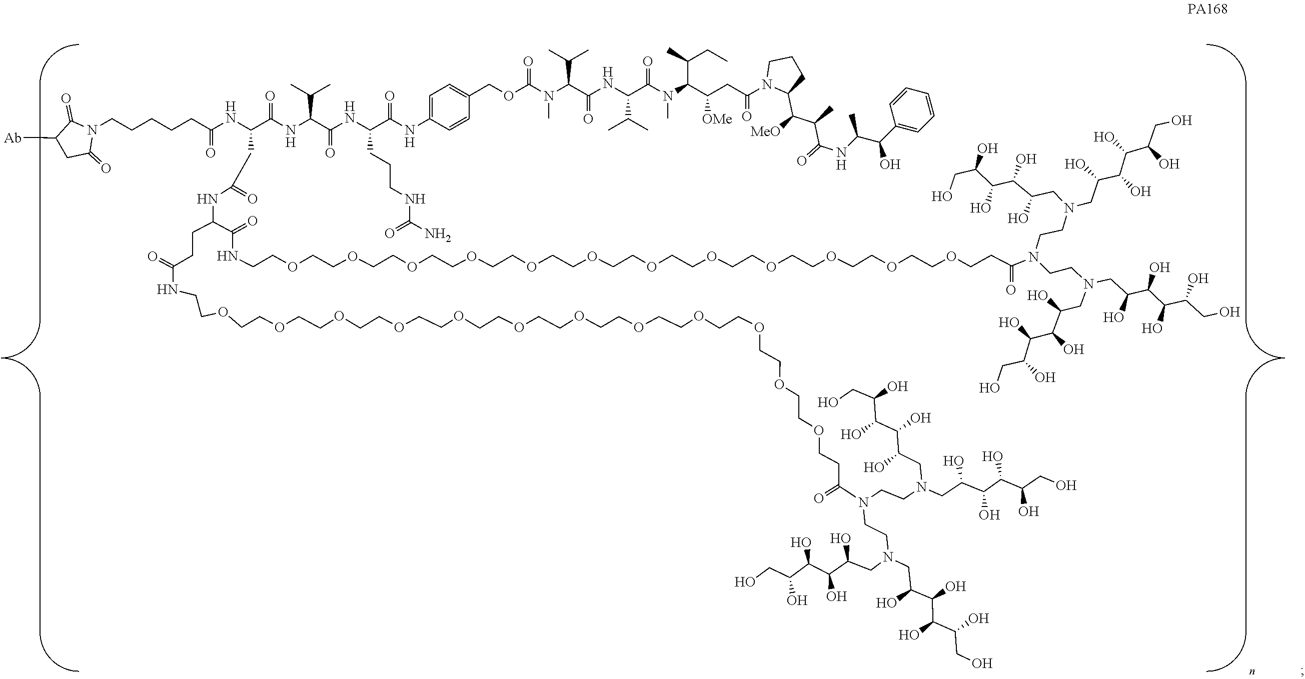

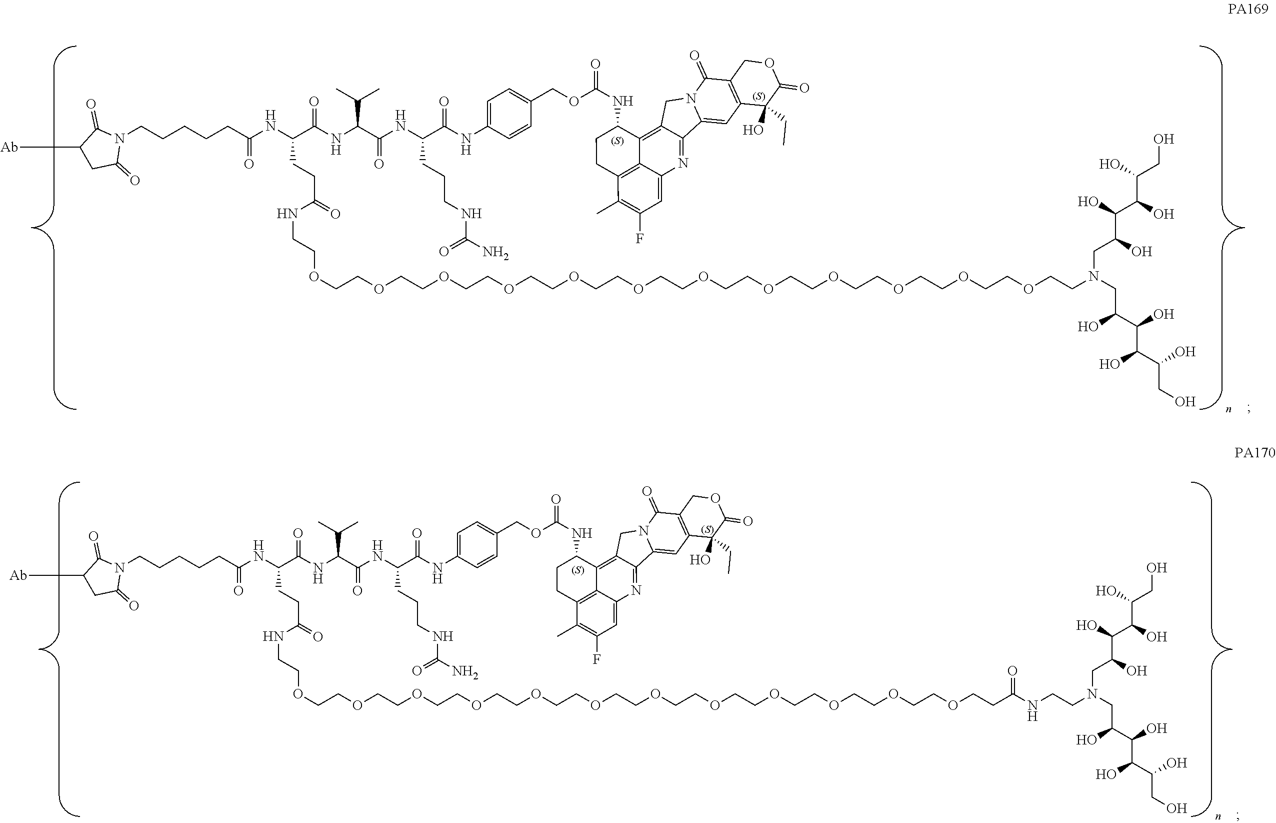

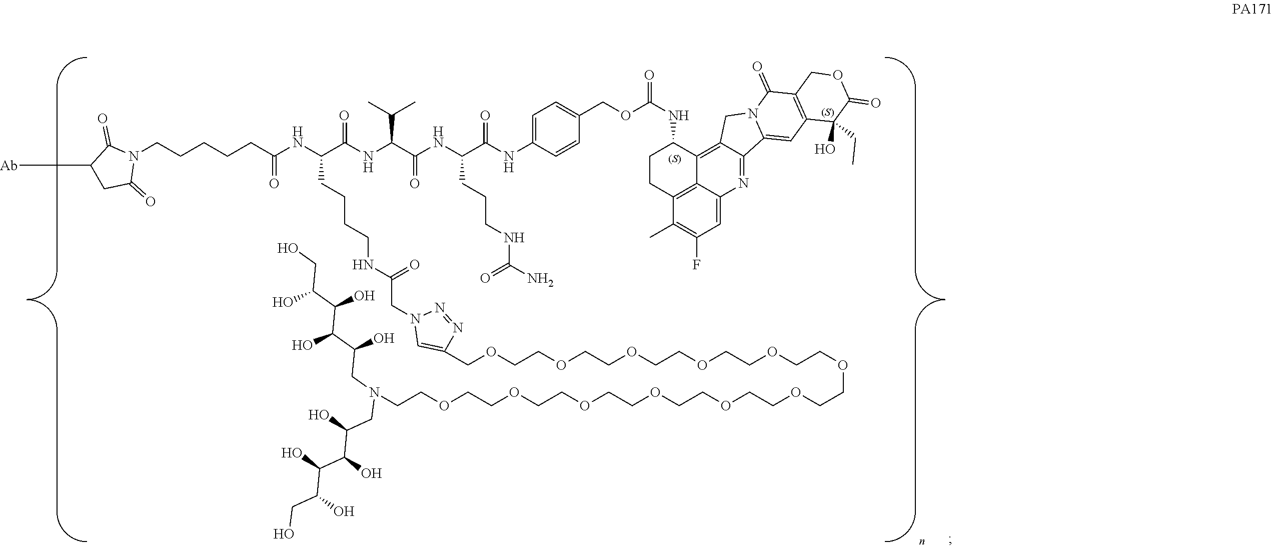

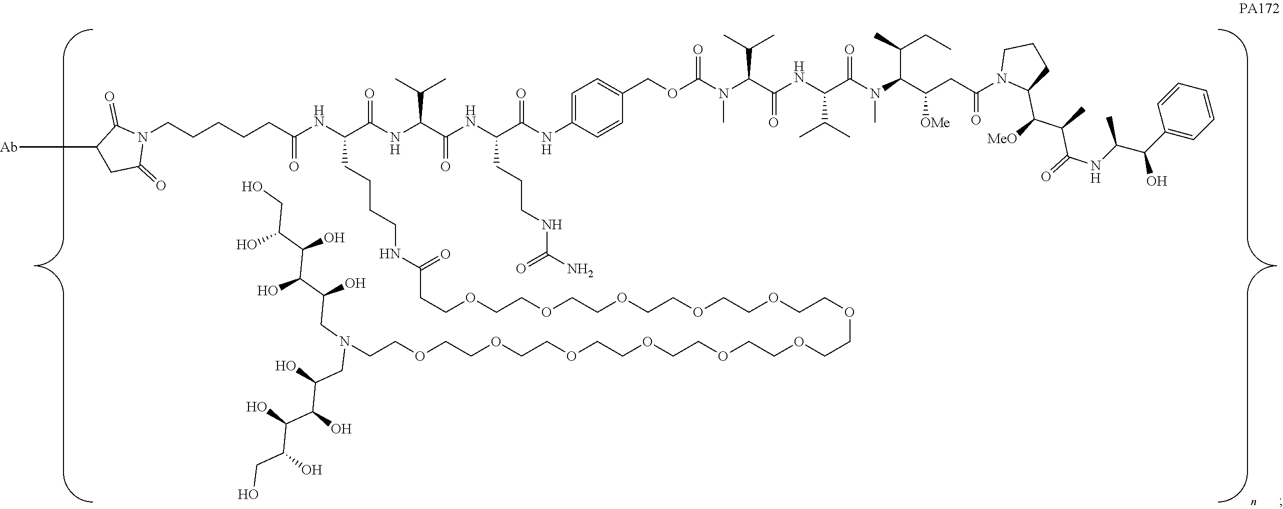

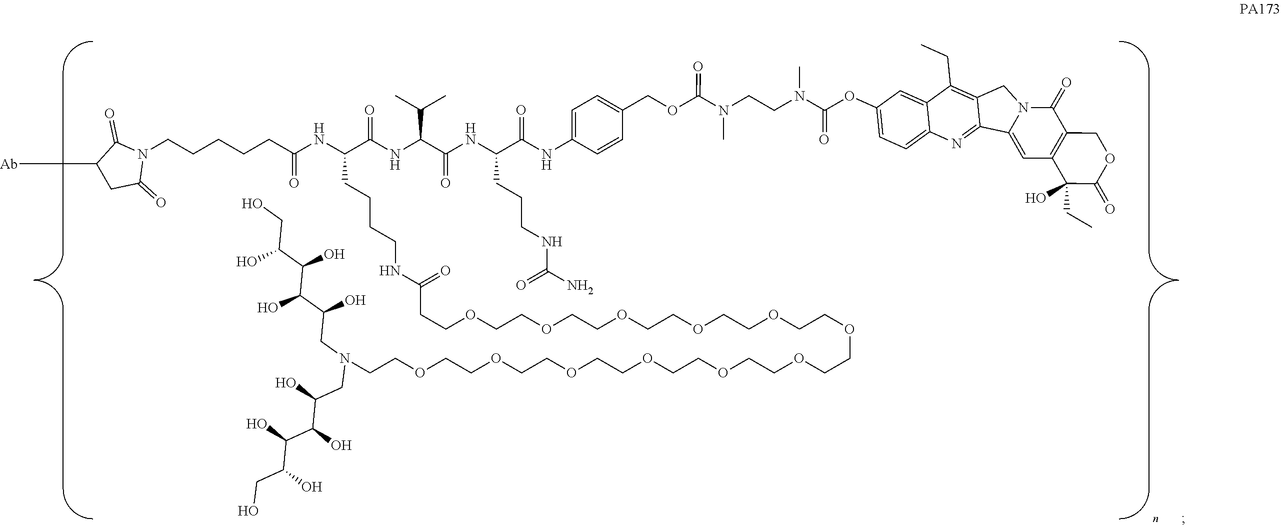

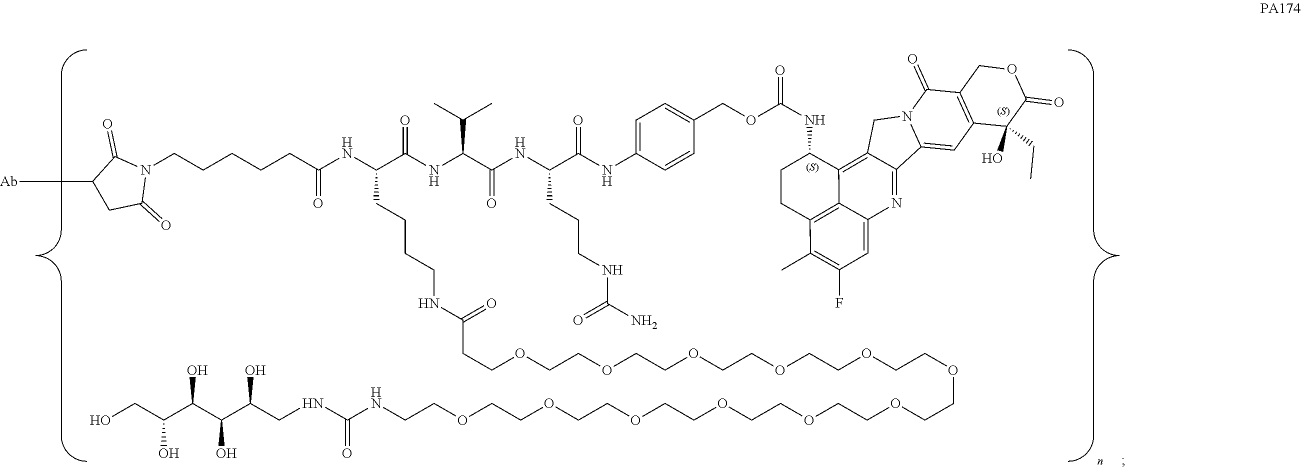

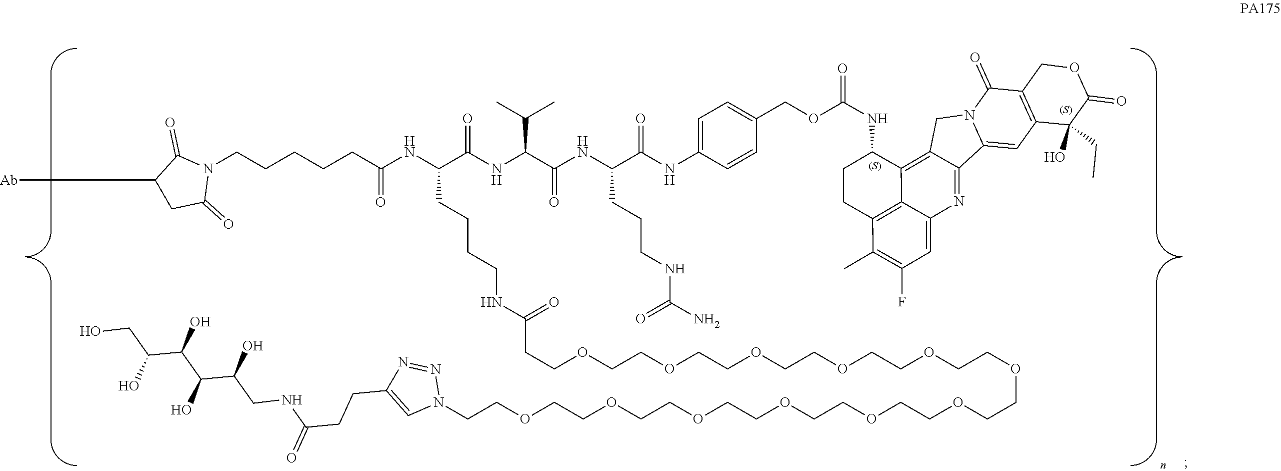

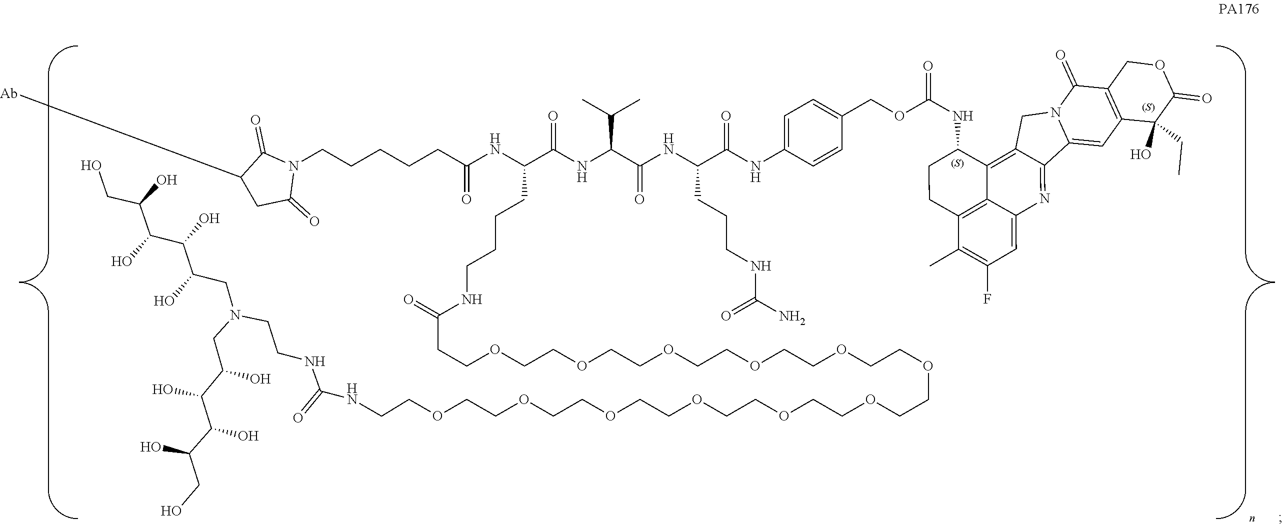

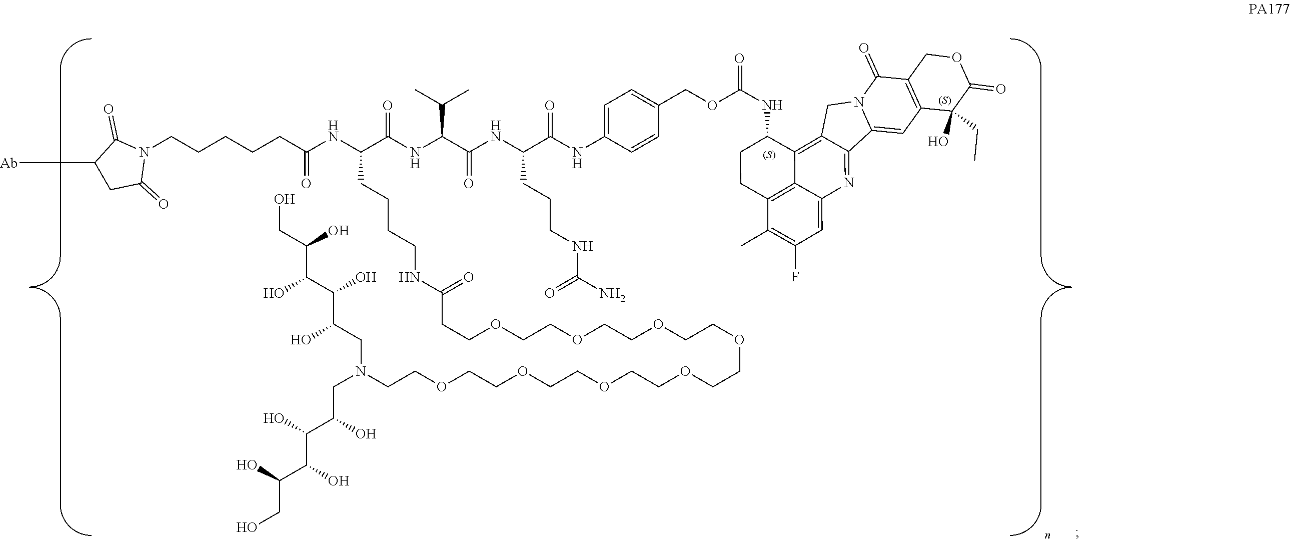

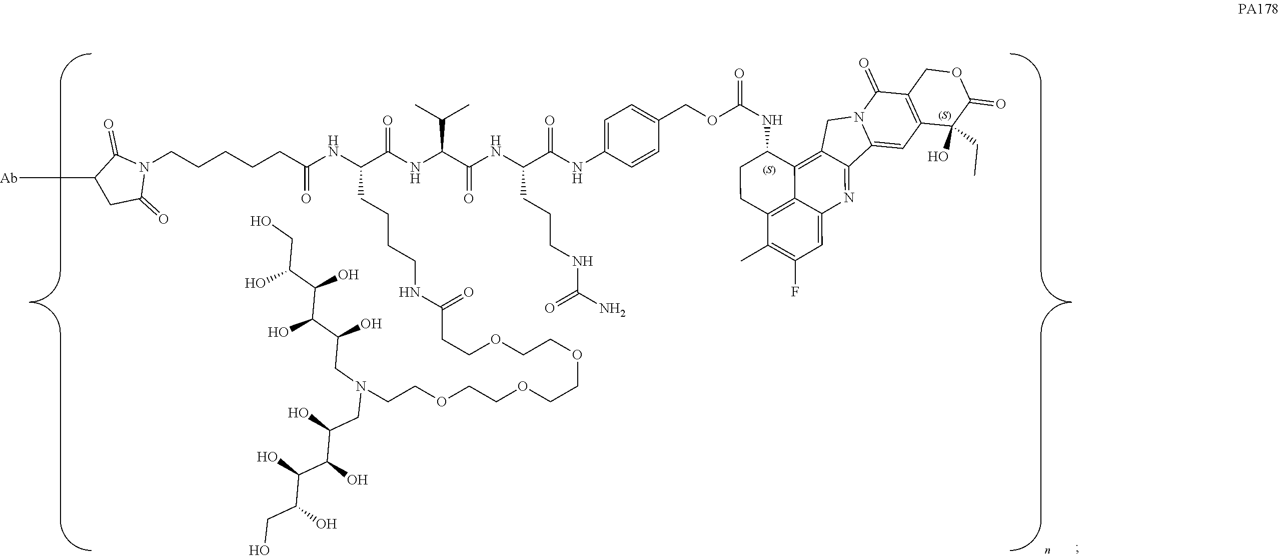

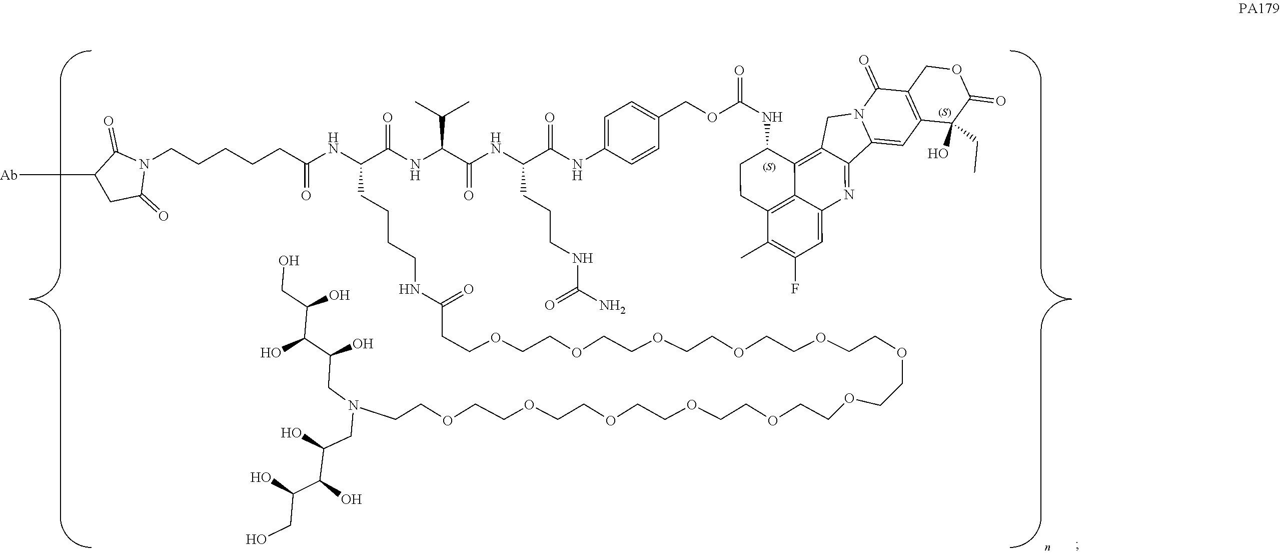

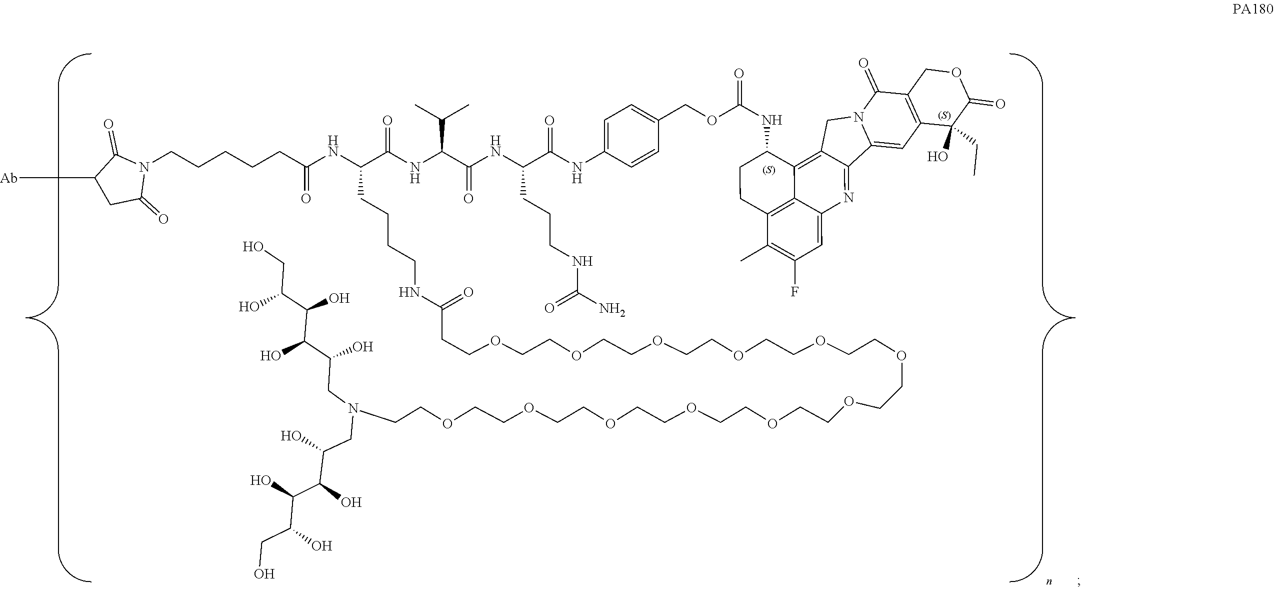

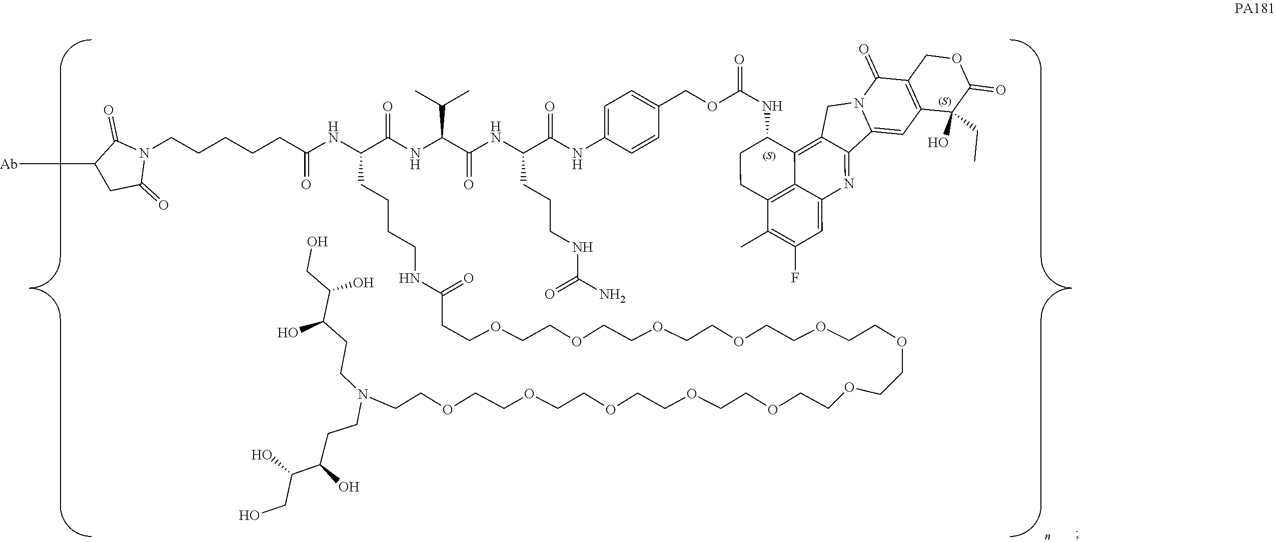

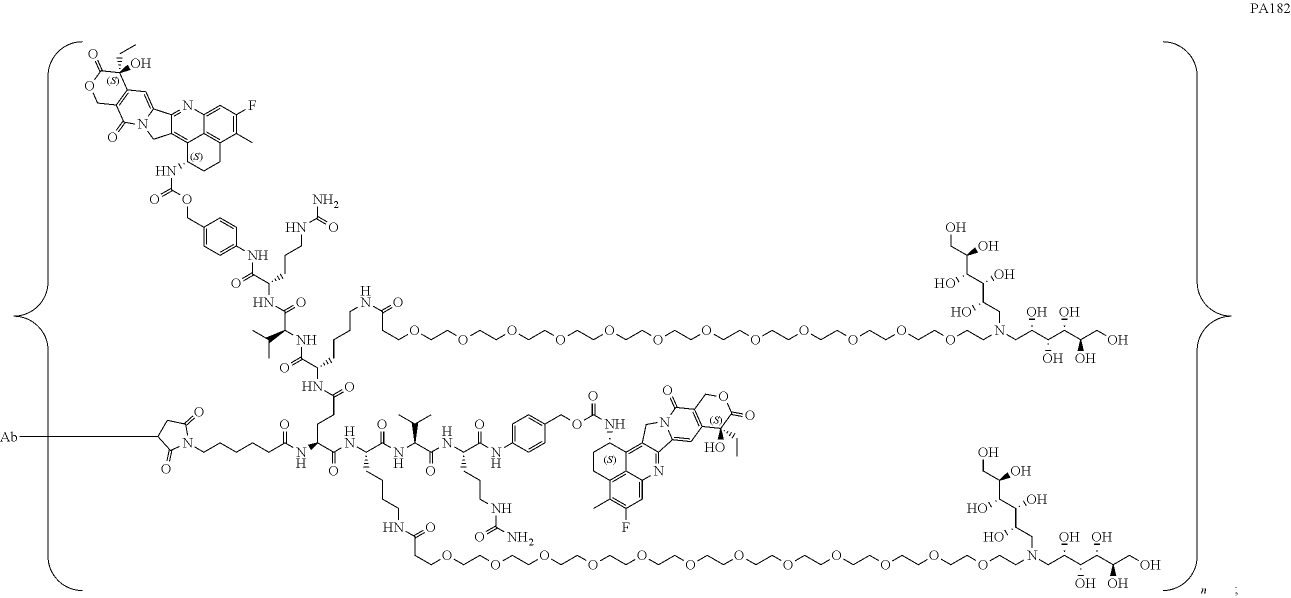

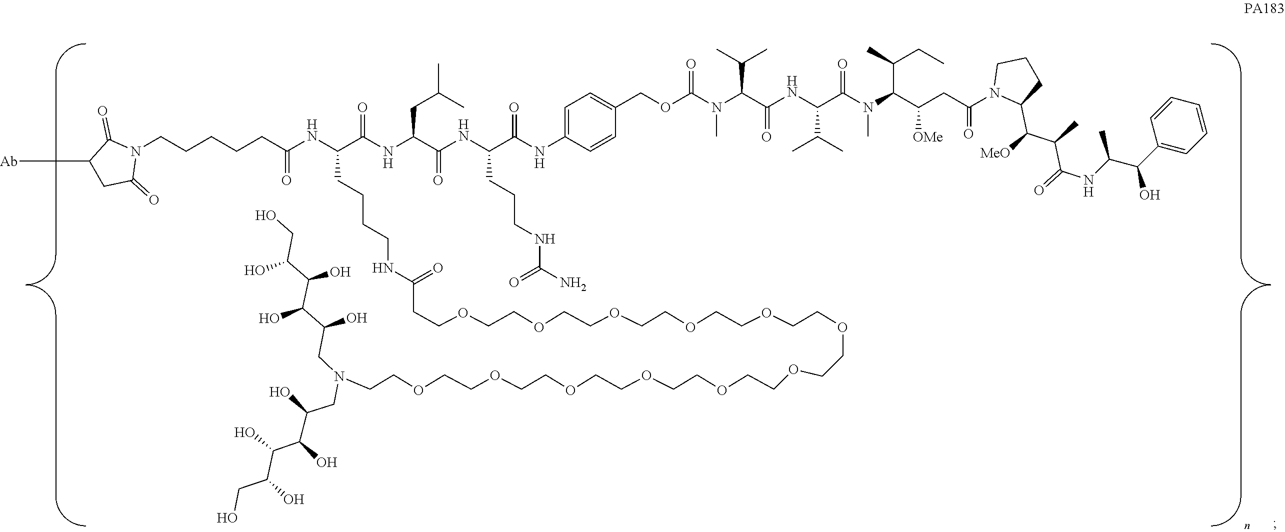

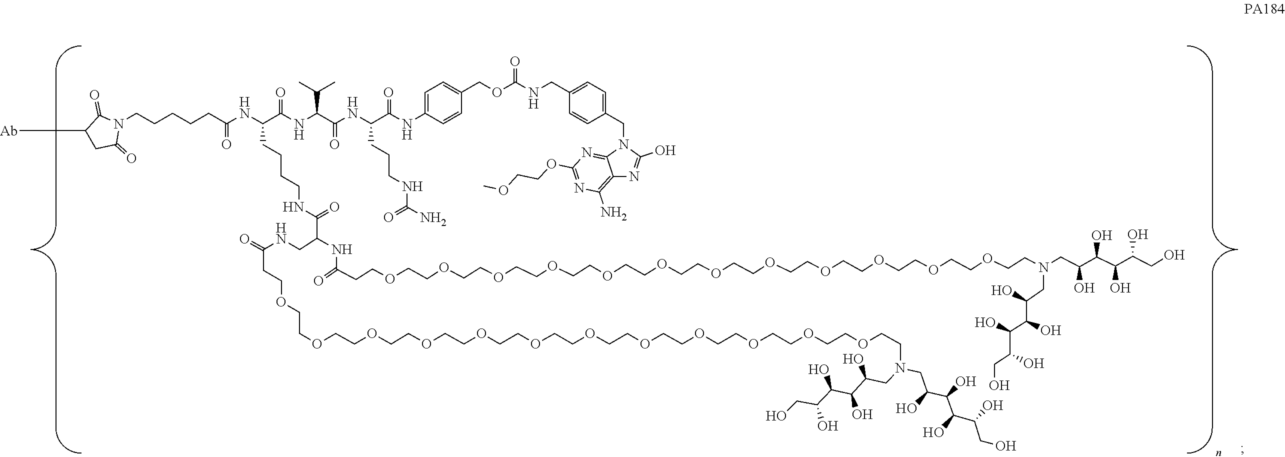

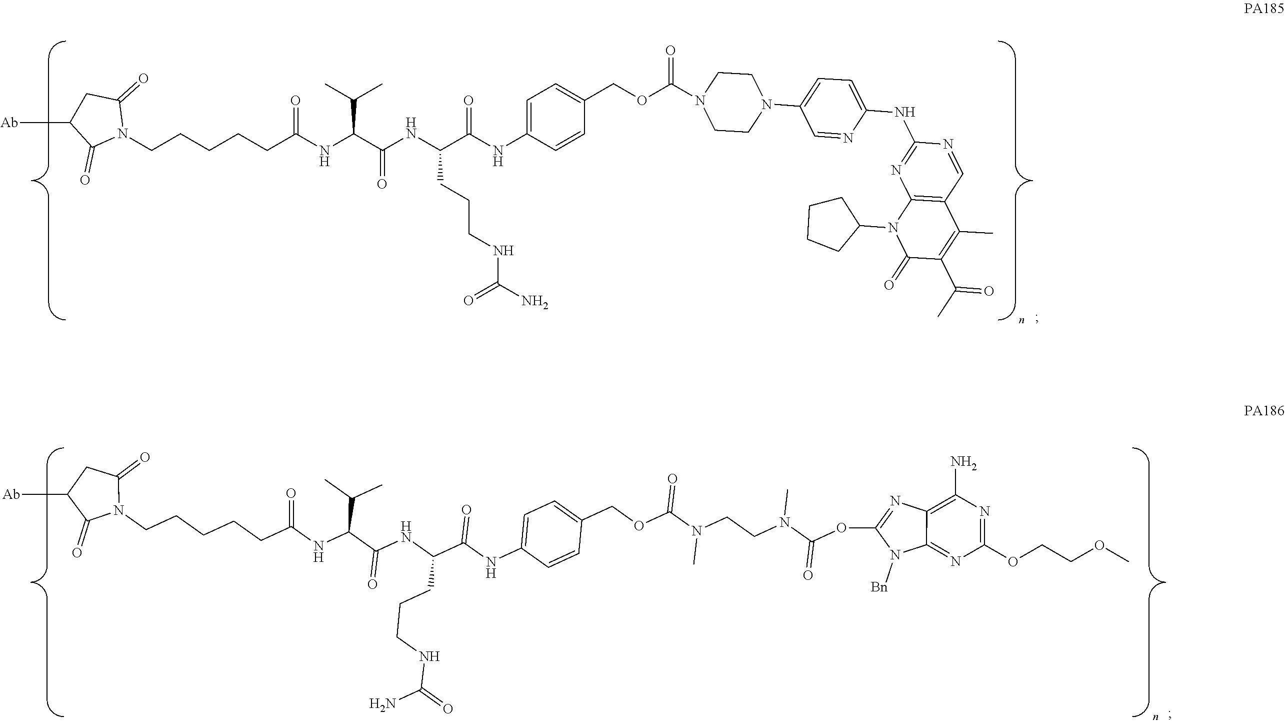

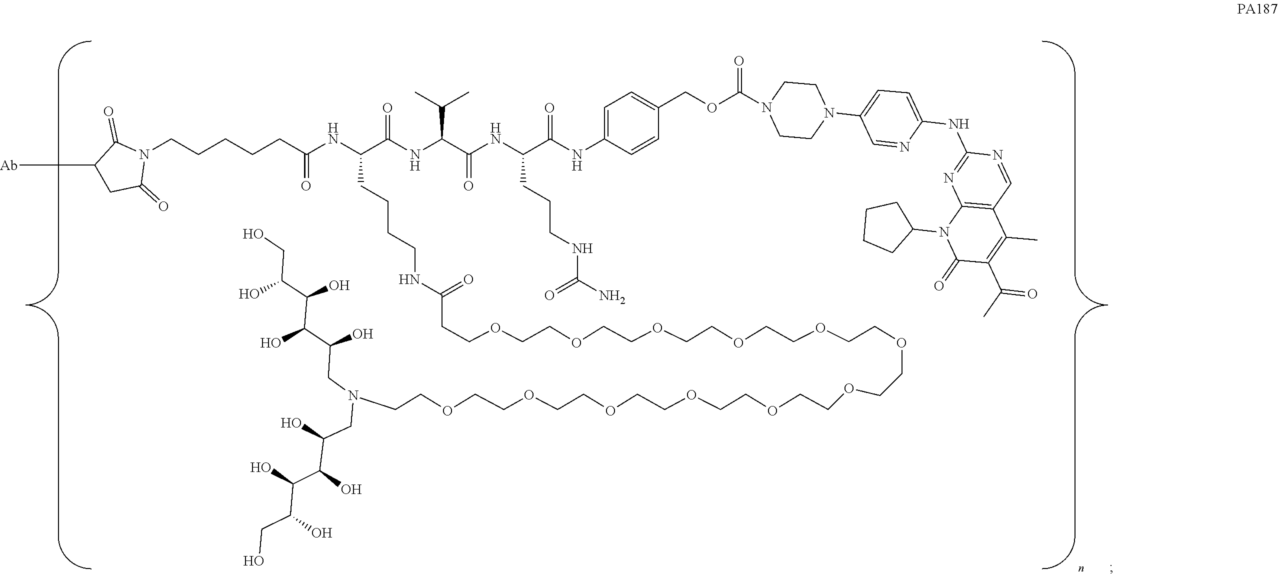

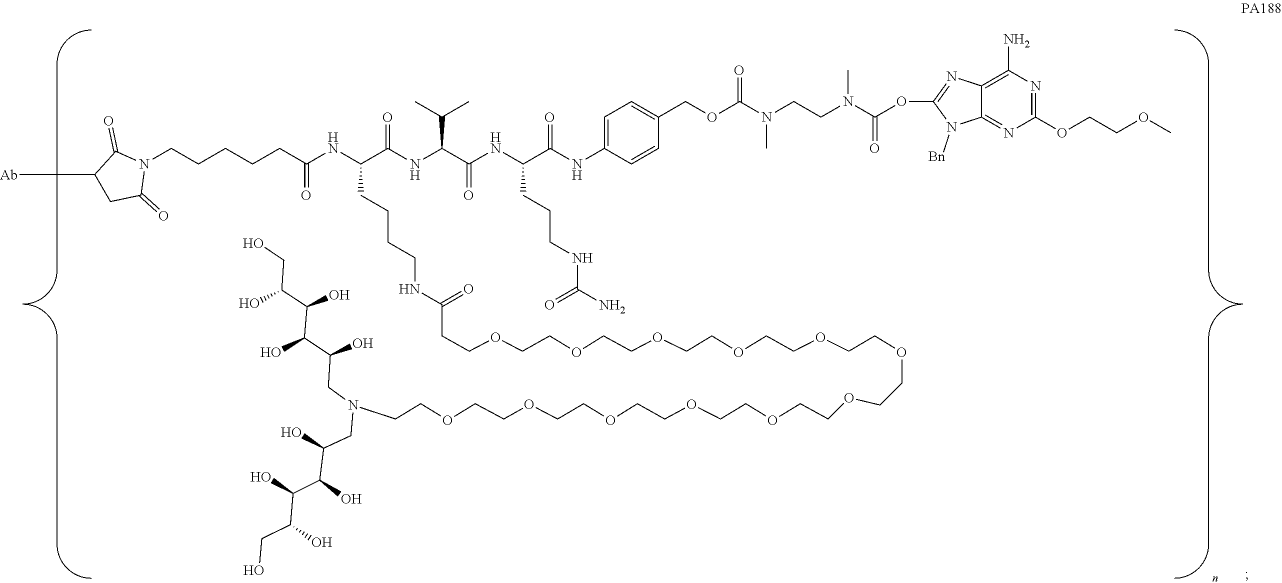

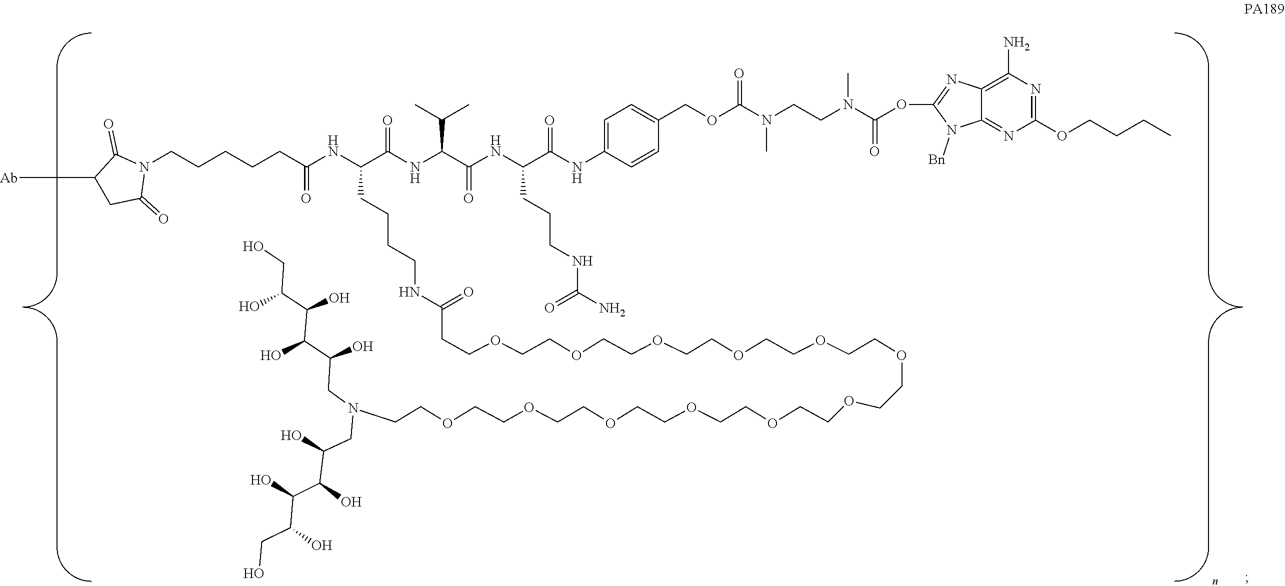

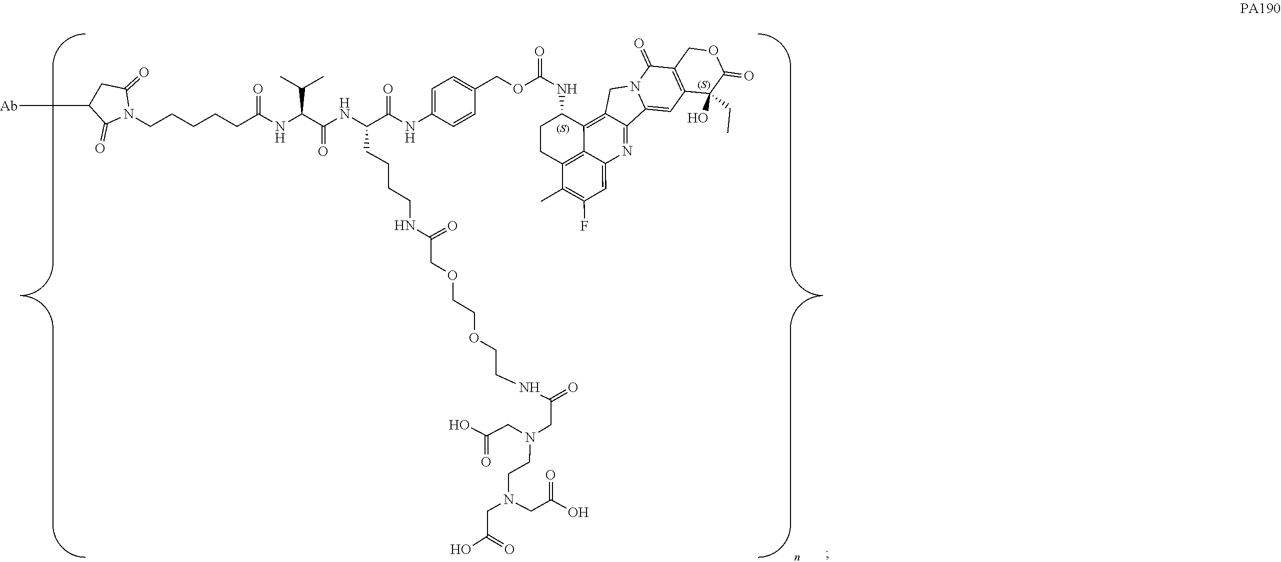

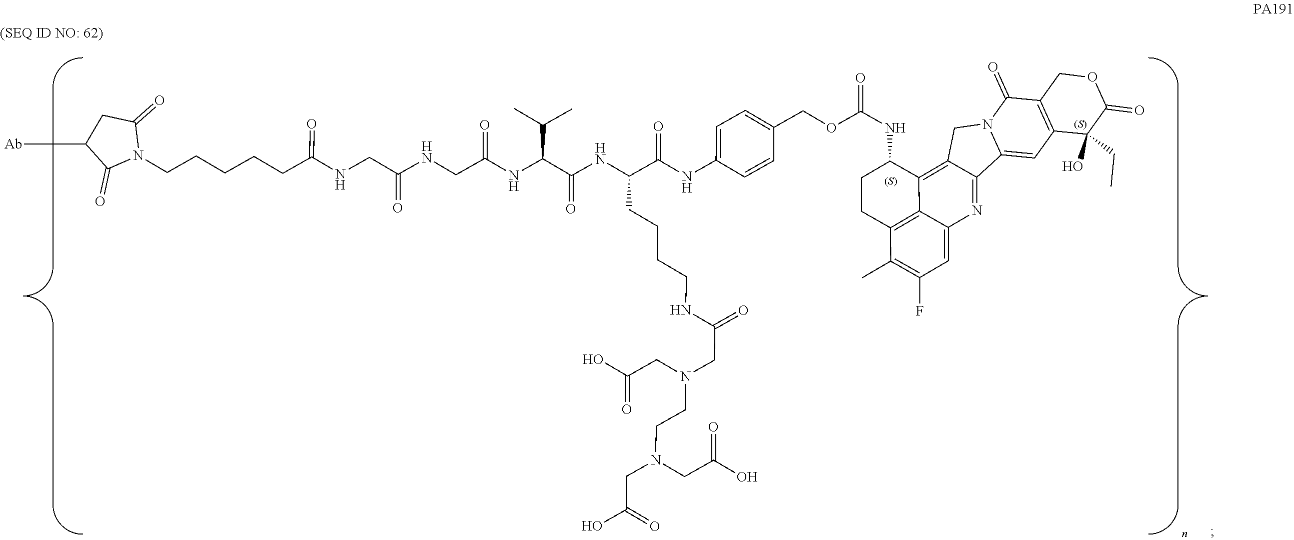

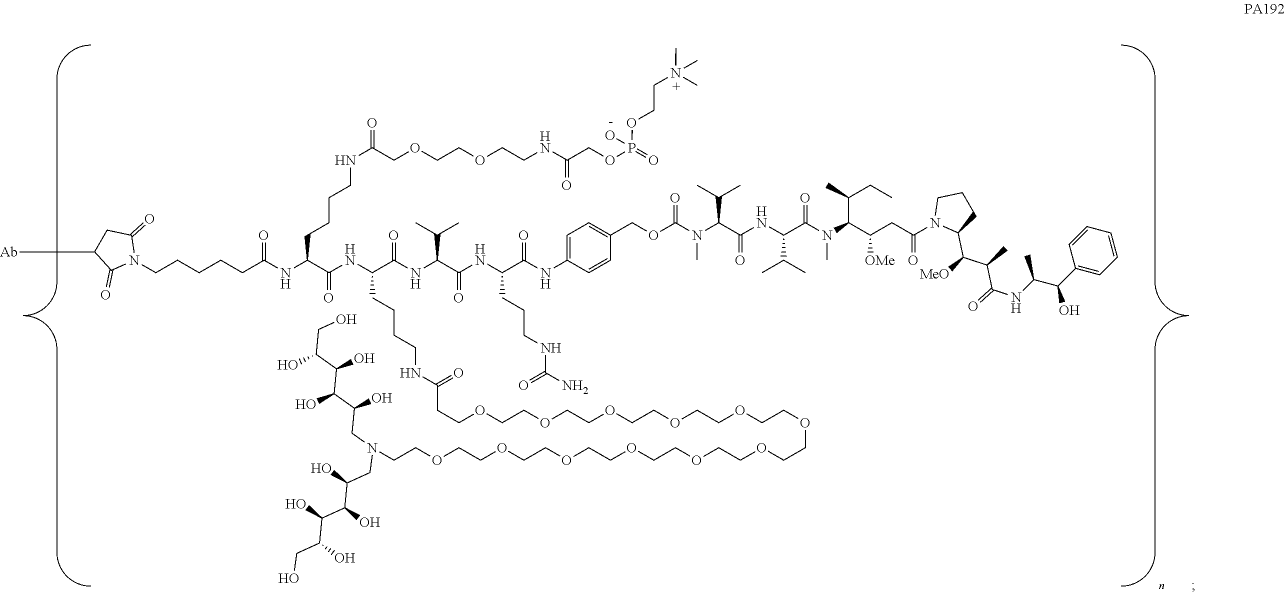

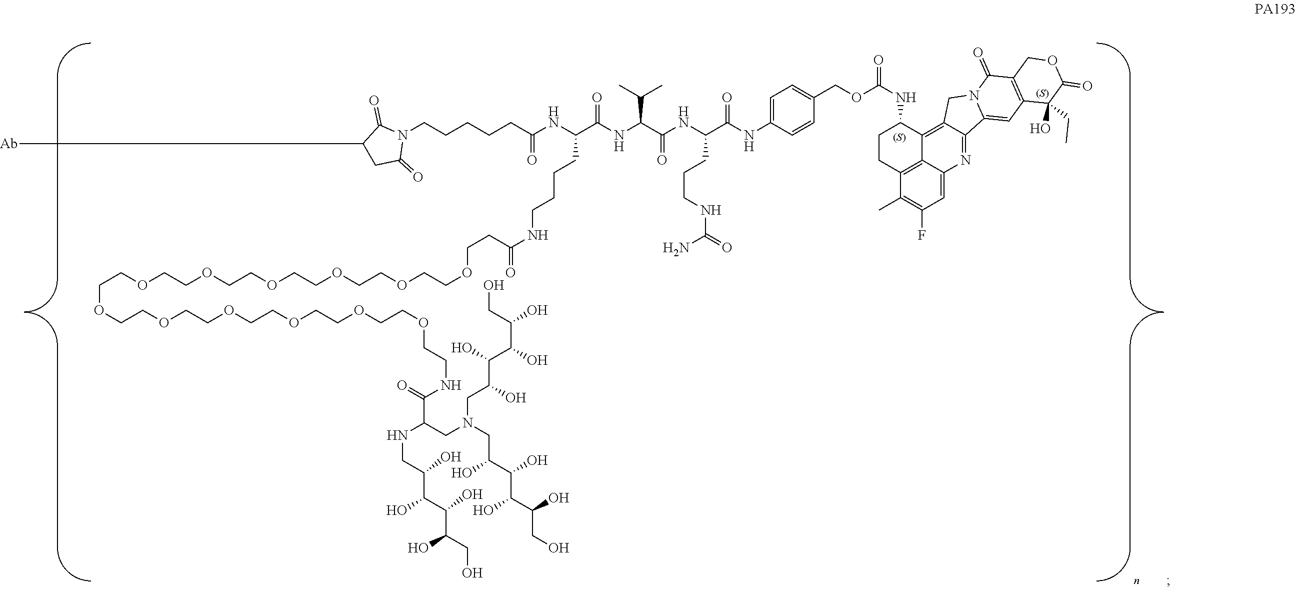

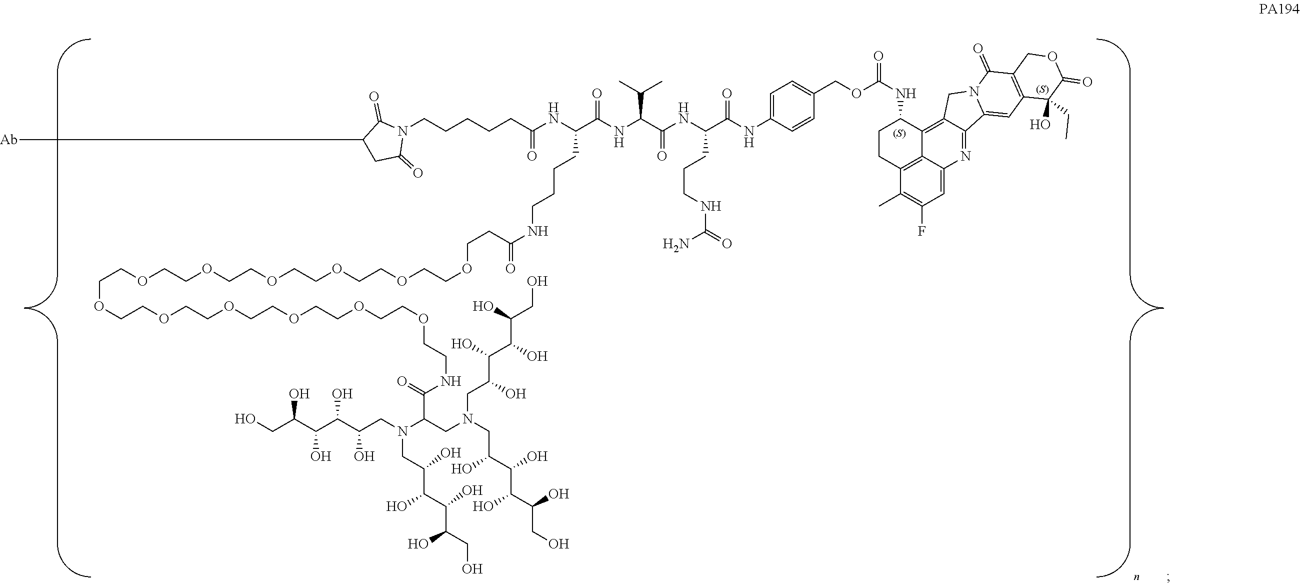

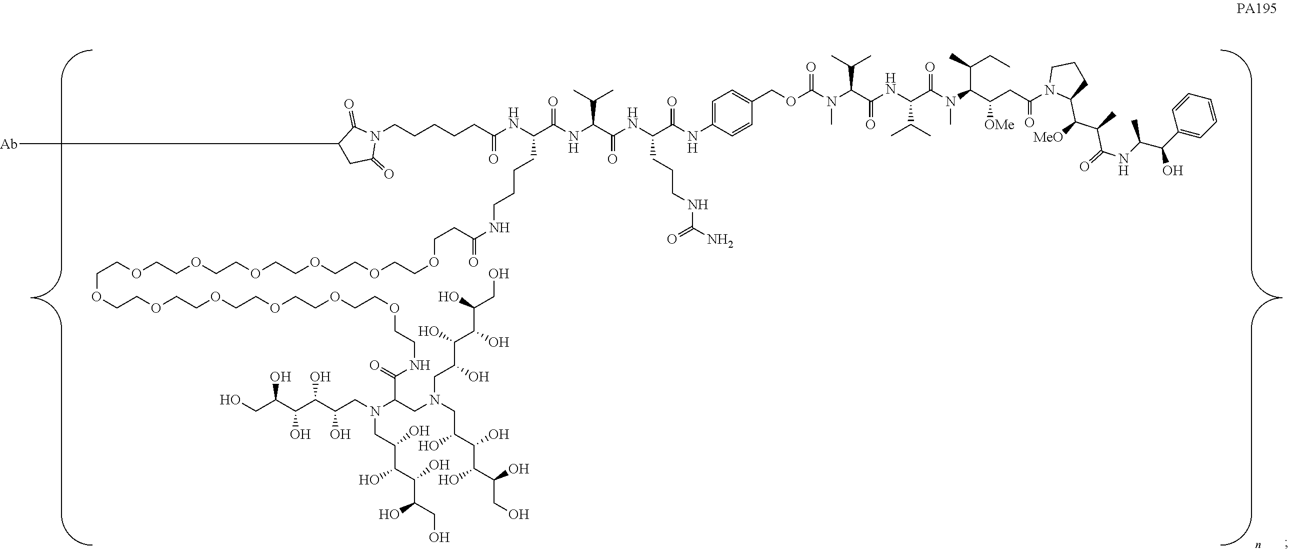

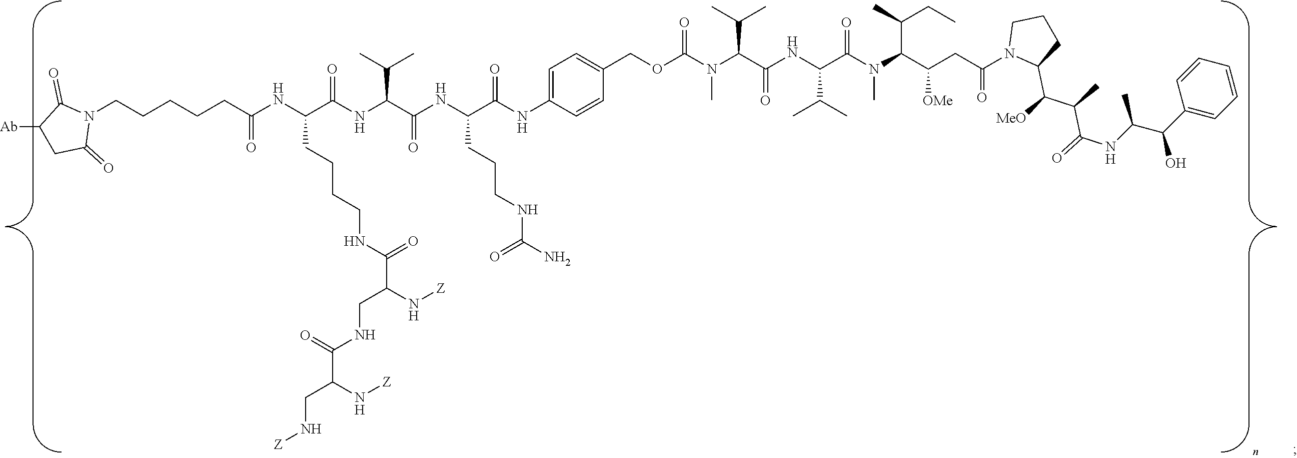

wherein each Z is attached at * and is individually selected from:

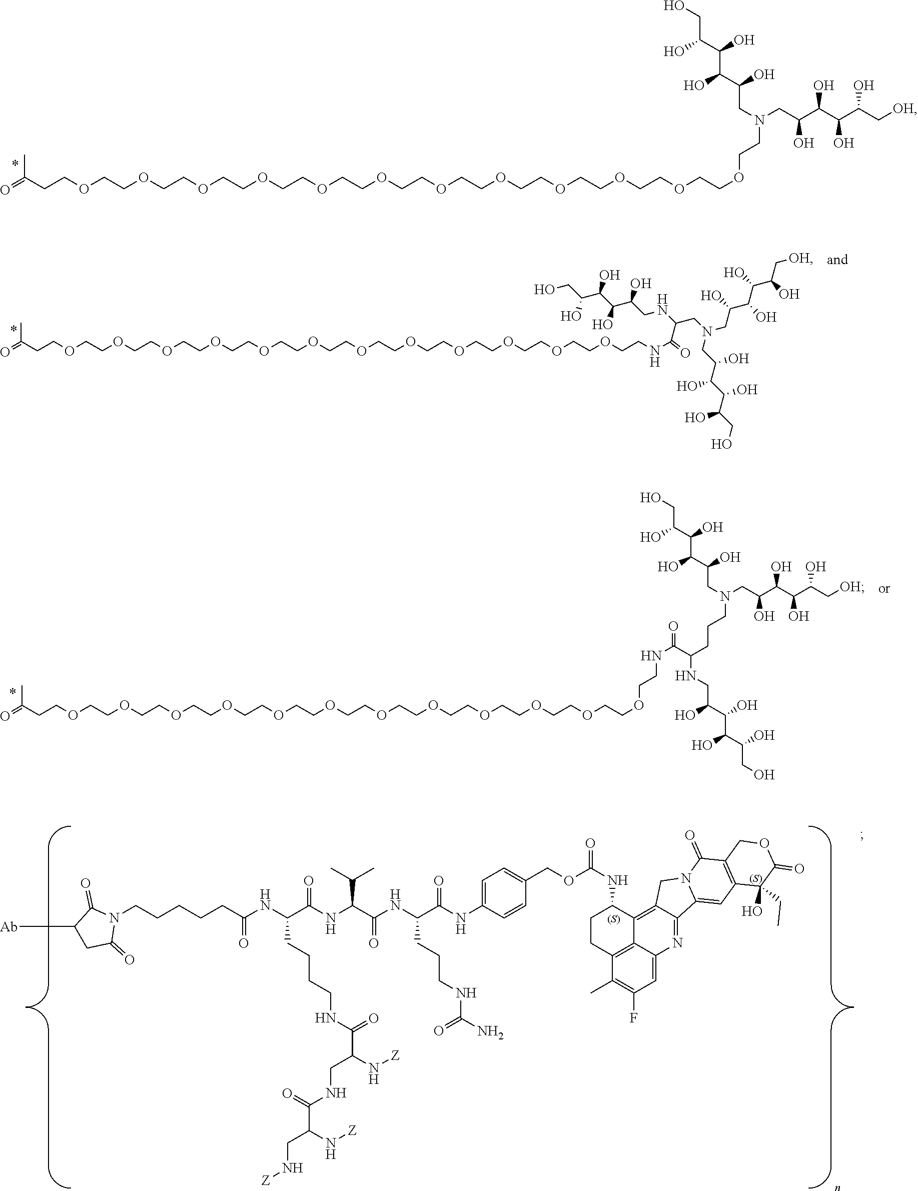

wherein each Z is attached at * and is individually selected from:

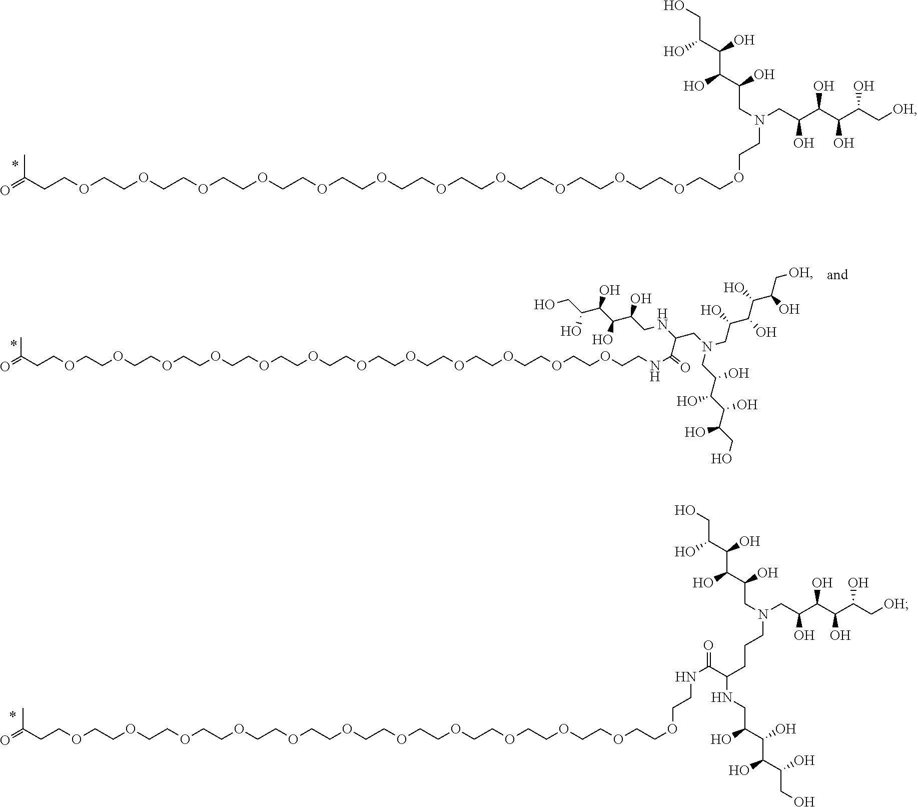

wherein Ab is a Targeting unit and n is pload.

In some embodiments, provided is a conjugate as described above wherein the Targeting unit binds to a target molecule such as such as CD19, CD20, CD30, CD33, CD70, LIV-1 or EGFRv3.

In some embodiments, provided is a conjugate as described above wherein the Targeting unit is selected from: a scFv1-ScFv2, a ScFv12-Fc-scFv22, a IgG-scFv, a DVD-Ig, a triomab/quadroma, a two-in-one IgG, a scFv2-Fc, a TandAb, and an scFv-HSA-scFv.

In some embodiments, provided is a conjugate as described above wherein the Targeting unit is a cancer associated antigen, such as CD19, CD20, CD30, CD33, CD38, CA125, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCH1, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4.

In some embodiments, provided is a conjugate as described above wherein the Targeting unit is an antibody, or fragment thereof, including rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab tiuxetan (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), tositumumab, ibritumomab, the CD20 antibodies 2F2 (HuMax-CD20), 7D8, IgM2C6, IgG1 2C6, 11B8, B1, 2H7, LT20, 1FS or AT80, daclizumab (Zenapax®), or anti-LHRH receptor antibodies such as clone A9E4, F1G4, AT2G7, GNRH03, or GNRHR2.

In some embodiments, provided is a conjugate as described above wherein the Targeting unit is antibody F131 and the Drug-Linker is LD038. In a specific embodiment, the Targeting unit is antibody F131 (VH SEQ ID NO: 26 and VL SEQ ID NO: 27).

In some embodiments, provided is a conjugate as described above wherein the Targeting unit is an antibody comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having amino acids sequences selected from the sets of amino acid sequences set forth in the group consisting of: (a) SEQ ID NO:30, SEQ ID NO:31, SEQ ID NO:32, SEQ ID NO:33, SEQ ID NO:34 and SEQ ID NO:35, respectively; and (b) SEQ ID NO:36, SEQ ID NO:31, SEQ ID NO:37, SEQ ID NO:38, SEQ ID NO:39 and SEQ ID NO:40, respectively. In certain embodiments, the VH and VL regions have amino acid sequences that are selected from the pairs of amino acid sequences set forth in the group consisting of: SEQ ID NO:26 and SEQ ID NO:27; respectively; and wherein the heavy and light chain framework regions are optionally modified with from 1 to 8 amino acid substitutions, deletions or insertions in the framework regions. In a specific embodiment, the antibody is F131 and the Drug-Linker is LD038.

In some embodiments, provided is a pharmaceutical composition comprising the any of the conjugates described herein and a pharmaceutically acceptable carrier.

In some embodiments, provided is a method of treating a subject in need thereof, comprising administering to the subject any of the conjugates described herein or any of the pharmaceutical compositions described herein, wherein the subject has cancer or an autoimmune disease and the conjugate binds to a target antigen associated with the cancer or autoimmune disease.

These and other aspects of the present invention may be more fully understood by reference to the following detailed description, non-limiting examples of specific embodiments and the appended drawings.

### FIGURES

FIG. 1A. In vitro cytotoxicity of an anti-huFOLR-1 conjugate on OV90 cells.

FIG. 1B. In vitro cytotoxicity of an anti-huFOLR-1 conjugate on OVCAR-3 cells.

FIG. 1C. In vitro cytotoxicity of an anti-huFOLR-1 conjugate on NCI-H292 cells.

FIG. 2. The in vivo activity of a PA038 conjugate of human anti-huFOLR1 antibody was tested. Mice with established OV90 xenografts about 117 mm³ were dosed with 4 times in 2 weeks by intravenous injection of 5 mg per kg of the conjugate or PBS starting day 8 after tumor cell inoculation. Mean tumor volumes in mm³ versus time (in days) after cell inoculation are plotted. (N=6, Mean±SEM)

FIG. 3. The in vivo activity of a PA038 conjugate of human anti-huFOLR1 antibody was tested. Mice with established NCI-H292 xenografts about 123 mm³ were dosed with 4 times in 2 weeks by intravenous injection of 5 mg per kg of the conjugate or PBS starting day 11 after cell inoculation. Mean tumor volumes in mm³ versus time (in days) after cell inoculation are plotted. (N=6, Mean±SEM)

FIG. 4. The in vivo activity of PA038 conjugate of human anti-huFOLR1 antibodies was tested. Mice with established OV90 xenografts about 110 mm³ were dosed with 4 times in 2 weeks by intravenous injection of 5 mg per kg of conjugate or PBS starting day 13 after cell inoculation. Mean tumor volumes in mm3 versus time (in days) after cell inoculation are plotted. (N=6, Mean±SEM)

FIG. 5. The PK profiles of anti-huFOLR-1 conjugate F131-LD038 and naked Ab F131 were assessed at 3 mg/kg (N=3; Mean±SD).

FIG. 6. Comparison of anti-FOLR1 antibody binding to Hela cells.

FIG. 7. Comparison of anti-FOLR1 antibody binding ability to RPTEC/TERT1 cells.

FIG. 8. Dose-dependent binding of anti-FOLR1antibodies to Hela cells.

FIG. 9. Dose-dependent binding of anti-FOLR1 antibodies to RPTEC/TERT1 cells.

FIG. 10. Internalization of anti-FOLR1 antibodies into Hela cells.

FIG. 11. Internalization of anti-FOLR1 antibodies into RPTEC/TERT1 cells.

FIG. 12A. F131 Internalization in tumor cell lines.

FIG. 12B. F131-LD038 Internalization in tumor cell lines.

FIG. 13A. In vitro cell cytotoxicity on KB.

FIG. 13B. In vitro cell cytotoxicity on OVCAR3.

FIG. 13C. In vitro cell cytotoxicity on JEG-3.

FIG. 14A. In vivo efficacy of F131 and F131-LD038 in CDX on OVCAR-3.

FIG. 14B. In vivo efficacy of F131 and F131-LD038 in CDX on KB.

FIG. 14C. In vivo efficacy of F131 and F131-LD038 in CDX on HCC827.

FIG. 14D. In vivo efficacy of F131 and F131-LD038 in CDX on H441.

FIG. 14E. In vivo efficacy of F131 and F131-LD038 in CDX on OV90.

FIG. 15A. In vivo efficacy of F131-038 and other conjugates in CDX on KB.

FIG. 15B. In vivo efficacy of F131 conjugates in CDX on KB.

FIG. 16A. PK study in Rat model of F131 and conjugates.

FIG. 16B. PK study in Rat model of F131 and conjugates.

FIG. 16C. PK study in Rat model of F131 and conjugates.

FIG. 17A. F131-deruxtecan and F131-LD038 tolerability in the pilot cynomolgus toxicity study.

FIG. 17B. F131-deruxtecan and F131-LD038 tolerability in the pilot cynomolgus toxicity study.

FIG. 18. F131-deruxtecan and F131-LD038 PK in the pilot cynomolgus toxicity study.

### DEFINITIONS

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, an “epitope” refers to the amino acids conventionally bound by an immunoglobulin VHNL pair, such as the antibodies, antigen binding portions thereof and other binding agents described herein. Other binding agents comprise non-antibody scaffolds. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. “Specifically binds” as stated herein also refers to the ability of a molecule (e.g., an antibody or antigen binding portion thereof or non-antibody scaffold) described herein to bind to a target with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody, antigen binding portion or other binding agent and the concentration of target polypeptide. A person of ordinary skill in the art can determine appropriate conditions under which antibodies, antigen binding portions and other binding agents described herein selectively bind to a target molecule using any suitable methods, such as titration of an antibody or a binding agent in a suitable cell binding assay. A binding agent specifically bound to a target molecule is not displaced by a non-similar competitor. In certain embodiments, an antibody or antigen-binding portion thereof or other binding agent is said to specifically bind to a target molecule when it preferentially recognizes its target molecule in a complex mixture of proteins and/or macromolecules. Specific binding can be influenced by, for example, the affinity and avidity of the antibody, antigen binding portion or non-antibody scaffold and the concentration of target polypeptide. A person of ordinary skill in the art can determine appropriate conditions under which antibodies, antigen binding portions and non-antibody scaffolds described herein selectively bind to a target molecule using any suitable methods, such as titration of an antibody or a non-antibody scaffold in a suitable cell binding assay. A molecule specifically bound to a target molecule is not displaced by a non-similar competitor. In certain embodiments, an antibody or antigen-binding portion thereof or non-antibody scaffold is said to specifically bind to a target molecule when it preferentially recognizes its target molecule in a complex mixture of proteins and/or macromolecules.

Unless otherwise indicated, the term “alkyl” by itself or as part of another term refers to a substituted or unsubstituted straight chain or branched, saturated hydrocarbon having the indicated number of carbon atoms (e.g., “—C₁-C₅ alkyl”, “—C₁-C₈ alkyl” or “—C₁-C₁₀” alkyl refer to an alkyl group having from 1 to 5, 1 to 8, or 1 to 10 carbon atoms, respectively). Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

Unless otherwise indicated, “alkenyl” by itself or as part of another term refers to a C₂—C substituted or unsubstituted straight chain or branched, hydrocarbon with at least one site of unsaturation (i.e., a carbon-carbon, sp² double bond). Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless otherwise indicated, “alkynyl” by itself or as part of another term refers to a refers to C₂-C₈, substituted or unsubstituted straight chain or branched, hydrocarbon with at least one site of unsaturation (i.e., a carbon-carbon, sp triple bond. Examples include, but are not limited to: acetylenic and propargyl.

Unless other indicated, “alkylene” refers to a saturated, branched or straight chain or hydrocarbon radical of 1-8 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

Unless otherwise indicated, “alkenylene” refers to an unsaturated, branched or straight chain hydrocarbon radical of 2-8 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

Unless otherwise indicated, “alkynylene” refers to an unsaturated, branched or straight chain or cyclic hydrocarbon radical of 2-8 carbon atoms, and having two monovalent radical centers derived by the removal of two hydrogen atoms from the same or two different carbon atoms of a parent alkyne. Typical alkynylene radicals include, but are not limited to: acetylene, propargyl, and 4-pentynyl.

Unless otherwise indicated, the term “heteroalkyl,” by itself or in combination with another term, refers to a substituted or unsubstituted stable straight or branched chain hydrocarbon, or combinations thereof, saturated and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group (i.e., as part of the main chain) or at the position at which the alkyl group is attached to the remainder of the molecule. The heteroatom Si may be placed at any position of the heteroalkyl group, including the position at which the alkyl group is attached to the remainder of the molecule. Examples of heteroalkyl include the following: —CH₂CH₂OCH₃, —CH₂CH₂NHCH₃, —CH₂CH₂N(CH₃)CH₃, —CH₂SCH₂CH₃, CH₂CH₂S(O)CH₃, —CH₂CH₂S(O)₂CH₃, and —Si(CH₃)₃, -. Up to two heteroatoms may be consecutive, such as, for example, —CH₂NHOCH₃ and CH₂OSi(CH₃)₃. In some embodiments, a C₁ to C₄ heteroalkyl has 1 to 4 carbon atoms and 1 or 2 heteroatoms and a C₁ to C₃ heteroalkyl has 1 to 3 carbon atoms and 1 or 2 heteroatoms.

Unless otherwise indicated, the terms “heteroalkenyl” and “heteroalkynyl” by themselves or in combination with another term, refers to a substituted or unsubstituted stable straight or branched chain alkenyl or alkynyl having from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of a heteroalkenyl or heteroalkynyl group (i.e., as part of the main chain) or at the position at which the alkyl group is attached to the remainder of the molecule. The heteroatom Si may be placed at any position of a heteroalkenyl or heteroalkynyl group, including the position at which the alkyl group is attached to the remainder of the molecule.

Unless otherwise indicated, the term “heteroalkylene” by itself or as part of another substituent refers to a substituted or unsubstituted divalent group derived from a heteroalkyl (as discussed above), as exemplified by —CH₂CH₂SCH₂CH₂— and —CH₂SCH₂CH₂NHCH₂—. In some embodiments, a C, to C₄ heteroalkylene has 1 to 4 carbon atoms and 1 or 2 heteroatoms and a C, to C₃ heteroalkylene has 1 to 3 carbon atoms and 1 or 2 heteroatoms. For heteroalkylene groups, heteroatoms can also occupy either or both of the chain termini. Still further, for alkylene and heteroalkylene linking groups, no orientation of the linking group is implied.

Unless otherwise indicated, the terms “heteroalkenylene” and “heteroalkynylene” by themselves or as part of another substituent refers to a substituted or unsubstituted divalent group derived from an heteroalkenyl or heteroalkynyl (as discussed above). In some embodiments, a C₂ to C₄ heteroalkenylene or heteroalkynylene has 1 to 4 carbon atoms. For heteroalkenylene and heteroalkynylene groups, heteroatoms can also occupy either or both of the chain termini. Still further, for alkylene and heteroalkenylene and heteroalkynylene linking groups, no orientation of the linking group is implied.

Unless otherwise indicated, a “C₃-C₈ carbocycle,” by itself or as part of another term, refers to a substituted or unsubstituted 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C₃-C₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C₃-C₈ carbocyclo”, by itself or as part of another term, refers to a substituted or unsubstituted C₃-C₈ carbocycle group defined above wherein another of the carbocycle groups' hydrogen atoms is replaced with a bond (i.e., it is divalent).

Unless otherwise indicated, a “C₃-C₁₀ carbocycle,” by itself or as part of another term, refers to a substituted or unsubstituted 3-, 4-, 5-, 6-, 7-, 8-, 9- or 10-membered monovalent, substituted or unsubstituted, saturated or unsaturated non-aromatic monocyclic, bicyclic or tricyclic carbocyclic ring derived by the removal of one hydrogen atom from a ring atom of a parent ring system. Representative —C₃-C₁₀ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. —C₃-C₁₀ carbocycles can further include fused cyclooctyne carbocycles, such as the fused cyclooctyne compounds disclosed in International Publication Number WO2011/136645 (the disclosure of which is incorporated by reference herein), including BCN (bicyclo[6.1.0]nonyne) and DBCO (Dibenzocyclooctyne).

Unless otherwise indicated, a “C₃-C₈ heterocycle,” by itself or as part of another term, refers to a substituted or unsubstituted monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a C₃-C₈ heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. Unless otherwise indicate, the term “heterocarbocycle” is synonymous with the terms “heterocycle” or “heterocyclo” as described herein.

Unless otherwise indicated, “C₃-C₈ heterocyclo”, by itself or as part of another term, refers to a substituted or unsubstituted C₃-C₈ heterocycle group defined above wherein one of the heterocycle group's hydrogen atoms is replaced with a bond (i.e., it is divalent).

Unless otherwise indicated, “aryl” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene” by itself or as part of another term, is an unsubstituted or substituted aryl group as defined above wherein one of the aryl group's hydrogen atoms is replaced with a bond (i.e., it is divalent) and can be in the ortho, meta, or para orientations.

Unless otherwise indicated, “heteroaryl” and “heterocycle” refer to a ring system in which one or more ring atoms is a heteroatom, e.g., nitrogen, oxygen, and sulfur. A heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Unless otherwise indicated, an “heteroarylene” by itself or as part of another term, is an unsubstituted or substituted heteroaryl group as defined above wherein one of the heteroaryl group's hydrogen atoms is replaced with a bond (i.e., it is divalent).

Unless otherwise indicated, “carboxyl” refers to COOH or COO⁻M⁺, where M⁺ is a cation.

Unless otherwise indicated, “oxo” refers to (C═O).

Unless otherwise indicated, “substituted alkyl” and “substituted aryl” mean alkyl and aryl, respectively, in which one or more hydrogen atoms are each independently replaced with a substituent. Typical substituents include, but are not limited to, —X, —R¹⁰, —O—, —OR¹⁰, —SR¹⁰, —S⁻, —NR¹⁰₂, —NR¹⁰₃, ═NR¹⁰, —CX₃, —CN, —OCN, —SCN, —N═C═O, —NCS, —NO, —NO₂, ═N₂, —N₃, —NR¹⁰ C(═O)R¹⁰, —C(═O)R¹⁰, —C(═O)NR¹⁰₂, —SO₃, —SO₃H, —S(═O)₂R¹⁰, —OS(═O)₂OR¹⁰, —S(═O)₂NR¹⁰, —S(═O)R¹⁰, —OP(═O)(OR¹⁰)₂, —P(═O)(OR)₂, —PO—₃, —PO₃H₂, —AsO₂H₂, —C(═O)R¹⁰, —C(═O)X, —C(═S)R¹⁰, —CO₂R¹⁰, —CO₂—, —C(═S)OR¹⁰, C(═O)SR¹⁰, C(═S)SR¹⁰, C(═O)NR¹⁰₂, C(═S)NR¹⁰₂, or C(═NR¹⁰)NR¹⁰₂, where each X is independently a halogen: —F, —C, —Br, or —I; and each R¹⁰ is independently —H, —C₁-C₂₀ alkyl, —C₆-C₂₀ aryl, —C₃-C₁₄ heterocycle, a protecting group or a prodrug moiety. Typical substitutents also include (═O). Alkylene, carbocycle, carbocyclo, arylene, heteroalkyl, heteroalkylene, heterocycle, and heterocyclo groups as described above may also be similarly substituted.

Unless otherwise indicated, “polyhydroxyl group” refers to an alkyl, alkylene, carbocycle or carbocyclo group including two or more, or three or more, substitutions of hydroxyl groups for hydrogen on carbon atoms of the carbon chain. In some embodiments, a polyhydroxyl group comprises at least three hydroxyl groups. In some embodiments, a polyhydroxyl group comprises carbon atoms containing only one hydroxyl group per carbon atom. A polyhydroxyl group may contain one or more carbon atoms that are not substituted with hydroxyl. A polyhydroxyl group may have each carbon atom substituted with a hydroxyl group. Examples of polyhydroxyl group includes linear (acyclic) or cyclic forms of monosaccharides such as C6 or C5 sugars, such as glucose, ribose, galactose, mannose, arabinose, 2-deoxyglucose, glyceraldehyde, erythrose, threose, xylose, lyxose, allose, altrose, gulose, idose, talose, aldose, and ketose, sugar acids such as gluconic acid, aldonic acid, uronic acid or ulosonic acid, and an amino sugars, such as glucosamine, N-acetyl glucosamine, galactosamine, and N-acetyl galactosamine. In some embodiments, polyhydroxyl group includes linear or cyclic forms of disaccharides and polysaccharides.

Unless otherwise indicated by context, “optionally substituted” refers to an alkyl, alkenyl, alkynyl, alkylaryl, arylalkyl heterocycle, aryl, heteroaryl, alkylheteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects an alkene function group replaces two contiguous sp3 carbon atoms of an alkyl substituent, provided that the radical carbon of the alkyl moiety is not replaced, so that the optionally substituted alkyl is an unsaturated alkyl substituent.

Optional substituent replacing hydrogen(s) in any one of the foregoing substituents, moieties or groups is independently selected from the group consisting of aryl, heteroaryl, hydroxyl, alkoxy, aryloxy, cyano, halogen, nitro, fluoroalkoxy, and amino, including mono-, di- and tri-substituted amino groups, and the protected derivatives thereof, or is selected from the group consisting of —X, —OR′, —SR′, —NH₂, —N(R′)(R″), —N(R″)₃, ═NR, —CX₃, —CN, —NO₂, —NR′C(═O)H, —NR′C(═O)R, —NR′C(═O)R″, —C(═O)R′, —C(═O)NH₂, —C(═O)N(R′)R″, —S(═O)₂R″, —S(═O)₂NH₂, —S(═O)₂N(R′)R″, —S(═O)₂NH₂, —S(═O)₂N(R′)R″, —S(═O)₂OR′, —S(═O)R″, —OP(═O)(OR′)(OR″), —OP(OH)₃, —P(═O)(OR′)(OR″), —PO₃H₂, —C(═O)R′, —C(═S)R″, —CO₂R′, —C(═S)OR″, —C(═O)SR′, —C(═S)SR′, —C(═S)NH₂, —C(═S)N(R′)(R″)₂, —C(═NR′)NH₂, —C(═NR′)N(R′)R″, and salts thereof, wherein each X is independently selected from the group consisting of a halogen: —F, —Cl, —Br, and —I; and wherein each R″ is independently selected from the group consisting of C₁-C₂₀ alkyl, C₂-C₂₀ alkenyl, C₂-C₂₀ alkynyl, C₆-C₂₄ aryl, C₃-C₂₄ heterocyclyl (including C₅-C₂₄ heteroaryl), a protecting group, and a prodrug moiety or two of R″ together with the heteroatom to which they are attached defines a heterocyclyl; and R′ is hydrogen or R″, wherein R″ is selected from the group consisting of C₁-C₂₀ alkyl, C₆-C₂₄ aryl, C₃-C₂₄ heterocyclyl (including C₅-C₂₄ heteroaryl), and a protecting group.

Typically, optional substituents are selected from the group consisting of —X, —OH, —OR″, —SH, —SR″, —NH₂, —NH(R″), —NR′(R″)₂, —N(R″)₃, ═NH, ═NR″, —CX₃, —CN, —NO₂, —NR′C(═O)H, NR′C(═O)R″, —CO₂H, —C(═O)H, —C(═O)R″, —C(═O)NH₂, —C(═O)NR′R″— —S(═O)₂R″, —S(═O)₂NH₂, —S(═O)₂N(R′)R″, —S(═O)₂NH₂, —S(═O)₂N(R′)(R″), —S(═O)₂OR′, —S(═O)R″, —C(═S)R″, —C(═S)NH₂, —C(═S)N(R′)R″, —C(═NR′)N(R″)₂, and salts thereof, wherein each X is independently selected from the group consisting of —F and —Cl, R″ is typically selected from the group consisting of C₁-C₆ alkyl, C₆-C₁ aryl, C₅-C₁₀, heterocyclyl (including C₁-C₁₀ heteroaryl), and a protecting group; and R′ independently is hydrogen, C₁-C₆ alkyl, C₆-C₁₀ aryl, C₃-C₁₀, heterocyclyl (including C₅-C₁₀ heteroaryl), and a protecting group, independently selected from R″. More typically, substituents are selected from the group consisting of —X, —R″, —OH, —OR″, —NH₂, —NH(R″), —N(R″)₂, —N(R″)₃, —CX₃, —NO₂, —NHC(═O)H, —NHC(═O)R″, —C(═O)NH₂, —C(═O)NHR″, —C(═O)N(R″)₂, —CO₂H, —CO₂R″, —C(═O)H, —C(═O)R″, —C(═O)NH₂, —C(═O)NH(R″), —C(═O)N(R″)₂, —C(═NR′)NH₂, —C(═NR′)NH(R″), —C(═NR′)N(R″)₂, a protecting group and salts thereof, wherein each X is —F, R″ is independently selected from the group consisting of C₁-C₆ alkyl, C₆-C₁₀, aryl, C₁-C₆ heteroaryl and a protecting group; and R′ is selected from the group consisting of hydrogen, C₁-C₆ alkyl and a protecting group, independently selected from R″.

The phrase “pharmaceutically acceptable salt,” as used herein, refers to pharmaceutically acceptable organic or inorganic salts of a compound (e.g., a Linker, Drug Linker, or a conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, linleate, gentisinate, fumarate, gluconate, glucuronate, saccharate, formate, benzoate, glutamate, methanesulfonate, ethanesulfonate, benzenesulfonate, toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The terms “statistically significant” or “significantly” refer to statistical significance and generally mean a two standard deviation (2SD) difference, above or below a reference value.

Other terms are defined herein within the description of the various aspects of the invention.

### DETAILED DESCRIPTION

Provided herein are Linkers that comprise a Polar unit, such as a Sugar unit, a PEG unit and/or a Carboxyl unit. Also provided are Targeting unit-Linkers, Drug Linkers, and conjugates thereof comprising Drug units, such as cytotoxic agents or immune modulatory agents, as further described herein.

In some embodiments, the Linkers have general formula (I), including a Stretcher unit (L1) attached to a Linker Subunit (L2) either directly or via an optional Amino Acid unit (AA), as shown in the following formula (I):

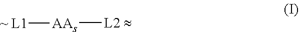

or a salt thereof, wherein s is 0 or 1, and the wavy (≈) lines indicate attachment sites for a Targeting unit (L) or a Drug unit (D). The Linkers comprise at least one Polar unit within the Amino Acid unit, the Linker Subunit L2, or both. Each Polar unit can be a Sugar unit, a PEG unit or a Carboxyl unit. A Linker can comprise at least one Sugar unit, at least one PEG unit, at least one Carboxyl unit, or combinations thereof. Linker Subunit L2 may have 1 to 4 attachment sites for Drug units. In some embodiments, Linker Subunit L2 has one attachment site for a Drug unit. In some embodiments, Linker Subunit L2 has two attachment sites for Drug units.

Also provided are conjugates of the Linker, comprising a Targeting unit (L) attached to at least one Linker, each Linker attached to at least one Drug unit (D), as shown in the following formula (II):

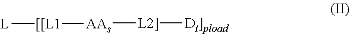

wherein L1, AA and L2 comprise a Linker and are as described above with respect to formula (I), s is 0 or 1, t is 1 to 4, and p^{load} is 1 to 20. The Linker comprises at least one Polar unit within the Amino Acid unit, the Linker Subunit L2, or both. Each Polar unit can be a Sugar unit, a PEG unit or a Carboxyl unit. A Linker can comprise at least one Sugar unit, at least one PEG unit, at least one Carboxyl unit, or combinations thereof. Linker Subunit L2 may have 1 to 4 attachment sites for Drug units. In some embodiments, Linker Subunit L2 has one attachment site for a Drug unit. In some embodiments, Linker Subunit L2 has two attachment sites for Drug units.

Also provided are Drug-Linkers as shown in the following formula (III).

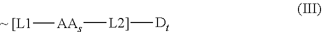

or a salt thereof, wherein L1, AA, L2 and D comprise a Linker and are as described above with respect to formula (II), s is 0 or 1, t is 1 to 4, and the wavy line indicates an attachment site for a Targeting unit. The Linker comprises at least one Polar unit within the Amino Acid unit, the Linker Subunit L2, or both. Each Polar unit can be a Sugar unit, a PEG unit or a Carboxyl unit. A Linker can comprise at least one Sugar unit, at least one PEG unit, at least one Carboxyl unit, or combinations thereof. Linker Subunit L2 may have 1 to 4 attachment sites for Drug units. In some embodiments, Linker Subunit L2 has one attachment site for a Drug unit. In some embodiments, Linker Subunit L2 has two attachment sites for Drug units.

Further provided are intermediates of Targeting unit-Linkers as shown in the following formula (IV):

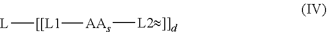

or a salt thereof, wherein L1, AA and L2 comprise a Linker, L, L1, AA and L2 are as described above with respect to formula (I), the s is 0 or 1, d is 1 to 20, and the double wavy line (≈) indicates an attachment site for a Drug unit. The Linker comprises at least one Polar unit within the Amino Acid unit, the Linker Subunit L2, or both. Each Polar unit can be a Sugar unit, a PEG unit or a Carboxyl unit. A Linker can comprise at least one Sugar unit, at least one PEG unit, at least one Carboxyl unit, or combinations thereof. Linker Subunit L2 may have 1 to 4 attachment sites for Drug units. In some embodiments, Linker Subunit L2 has one attachment site for a Drug unit. In some embodiments, Linker Subunit L2 has two attachment sites for Drug units.

### Polar Units

The Polar units (PU) provided herein include Sugar units, PEG units and Carboxyl units, as further described herein.

### Sugar Units (SU)

In some embodiments, Sugar units (SU) have the general formula (X):

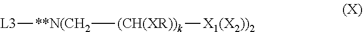

or a salt thereof, wherein each X is independently selected from NH or 0, each R is independently selected from hydrogen, acetyl, a monosaccharide, a disaccharide, and a polysaccharide, each X, is independently selected from CH₂ and C(O), each X₂ is independently selected from H, OH and OR, and k is 1 to 10. In some embodiments, each (CH₂—(CH(XR))ₖ—X₁(X₂)) is a monosaccharide. In some embodiments, the monosaccharide is a C₆ or C₅ sugar, such as glucose, ribose, galactose, mannose, arabinose, 2-deoxyglucose, glyceraldehyde, erythrose, threose, xylose, lyxose, allose, altrose, gulose, idose talose, aldose, ketose, a sugar acid such as gluconic acid, aldonic acid, uronic acid or ulosonic acid, or an amino sugar, such as glucosamine, N-acetyl glucosamine, galactosamine, and N-acetyl galactosamine. Suitable disaccharides include sucrose, lactose, and maltose. Suitable polysaccharides include maltotriose, raffinose, kestose, starch, cellulose, and glycogen. The stereochemistry at the anomeric C-1 position can be either alpha or beta.

L3 has the following general formula (XI):

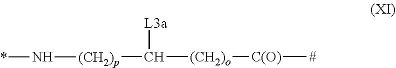

wherein L3a is selected from C₁-C₁₀ alkylene and polyethylene glycol (having from 1 to 26 ethylene glycol units), and p and o are independently 0 to 2, wherein L3a is covalently bound to the N atom marked with a ** in formula (X). Each * and each # indicates an attachment site for another subunit of an Amino Acid unit (AA) or a Linker subunit (L2), a Stretcher unit (L1), or other component of a Linker, as described herein.

In some embodiments, a Sugar unit has the following formula (XII):

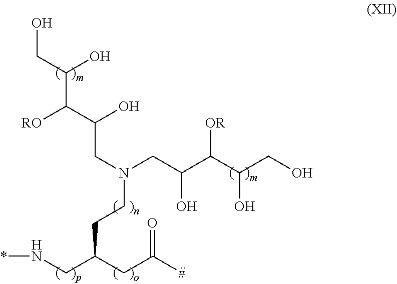

wherein R, p and o are as set forth above, n is from 0 to 4, and each m is independently from 1 to 4.

In some embodiments, a Sugar unit has the following formula (XIII):

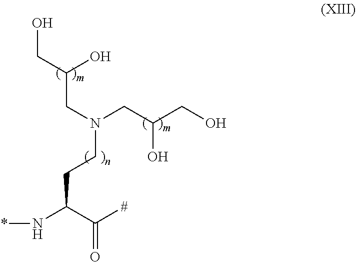

wherein n is from 0 to 4, and each m is independently from 1 to 4.

### PEG Units

In some embodiments, a Linker comprises a PEG unit. A PEG unit can be attached to a subunit of an Amino Acid unit or a portion of a Linker Subunit L2. A subunit of an Amino Acid unit can be, for example, an alpha, beta or gamma amino acid, or a derivative thereof. In some embodiments, a PEG unit can be attached to a Stretcher unit.

In some embodiments, a PEG unit has the following general formula —(CH₂CH₂O)ₙ₂₀—R²⁴, wherein R²⁴ is H or C₁-C₆ alkyl and n20 is 1 to 26. In some n20 is 12 and R²⁴ is methyl.

In some embodiments, a PEG unit has the following general formula (XX):

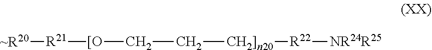

or a salt thereof, wherein R²⁰ is a functional group for attachment to a subunit of an Amino Acid unit and/or a portion of a Linker Subunit L2; R²¹ and R²² are each, independently, optional C₁-C₃ alkylene; R²⁴ and R²⁵ are as set forth below; the wavy line (˜) indicates an attachment site; and n20 is 1 to 26. In some embodiments, R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof. Suitable protecting groups include carboxylic acid protecting groups, amine protecting groups, and sulfonyl protecting groups typically used in the art.

In some embodiments, a PEG unit has the following general formula (XX):

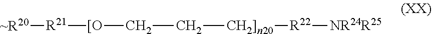

or a salt thereof, wherein R²⁰ is a functional group for attachment to a subunit of an Amino Acid unit and/or a portion of a Linker Subunit L2; R²¹ and R²² are each, independently, optional C₁-C₃ alkylene; R²⁴ and R²⁵ are as set forth below; the wavy line (˜) indicates an attachment site; and n20 is 1 to 26. In some embodiments, R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof. Suitable protecting groups include carboxylic acid protecting groups, amine protecting groups, and sulfonyl protecting groups typically used in the art.

In some embodiments, a PEG unit has the following general formula (XXI):

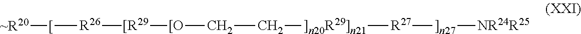

or a salt thereof, wherein R²⁰ is a functional group for attachment to a subunit of an Amino Acid unit and/or a portion of a Linker Subunit L2; R²⁶ and R²⁷ are each optional C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— or —C(O)—C₁-C₁₂ alkylene-NH—; R²⁴ and R²⁵ are as set forth below; each R²⁹ is optional and independently selected from —C(O)—, —NH—, —C(O)—C₁-C₆ alkenylene-, —NH—C₁-C₆ alkenylene-, —C₁-C₆ alkenylene-NH— and —C₁-C₆ alkenylene-C(O)—; the wavy line (˜) indicates an attachment site; n20 is 1 to 26; n21 is 1 to 4; and n27 is 1 to 3. In some embodiments, R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof. Suitable protecting groups include carboxylic acid protecting groups, amine protecting groups, and sulfonyl protecting groups typically used in the art.

In some embodiments, a PEG unit has the following general formula (XXI):

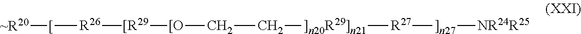

or a salt thereof, wherein R²⁰ is a functional group for attachment to a subunit of an Amino Acid unit and/or a portion of a Linker Subunit L2; R²⁶ and R²⁷ are each optional C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— or —C(O)—C₁-C₁₂ alkylene-NH—; R²⁴ and R²⁵ are as set forth below; each R²⁹ is optional and independently selected from —C(O)—, —NH—, —C(O)—C,—C₆ alkenylene-, —NH—C₁-C₆ alkenylene-, —C₁-C₆ alkenylene-NH—, —C₁-C₆ alkenylene-C(O)—, —NH(CO)NH—, and triazole; the wavy line (˜) indicates an attachment site; n20 is 1 to 26; n21 is 1 to 4; and n27 is 1 to 3. In some embodiments, R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof. Suitable protecting groups include carboxylic acid protecting groups, amine protecting groups, and sulfonyl protecting groups typically used in the art.

In some embodiments of the PEG units of formula (XX) and (XXI), R²⁴ and R²⁵ are each independently selected from H and polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); or —NR²⁴R²⁵ together from a C₃-C₈ heterocycle.

In some embodiments of the PEG units of formula (XX) and (XXI), one of R²⁴ and R²⁵ is selected from a H and polyhydroxyl group; substituted polyhydroxyl group; —C(O)— polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is polyethylene glycol, optionally having 1 to 24 ethylene glycol subunits.

In some embodiments of a PEG unit of formula (XX) or (XXI), both R²⁴ and R²⁵ are not H. In some embodiments of a PEG units of formula (XX) or (XXI), one of R²⁴ and R²⁵ is H.

In some embodiments of a PEG unit of formula (XX) and (XXI), R²⁴ and R²⁵ are each independently selected from H and polyhydroxyl group, provided that R²⁴ and R²⁵ are not both H. A polyhydroxyl group can be linear or branched. In some embodiments, the polyhydroxyl group includes at least three hydroxyl groups. In some embodiments, a polyhydroxyl group is a linear monosaccharide. As used herein, a linear monosaccharide refers to a ring open form of a monosaccharide. In some embodiments, a linear monosaccharide is a linear form of a C₆ or C₅ sugar, such as glucose, ribose, galactose, mannose, arabinose, 2-deoxyglucose, glyceraldehyde, erythrose, threose, xylose, lyxose, allose, altrose, gulose, idose talose, aldose, and ketose. In some embodiments, a linear monosaccharide can further include a sugar acid, such as gluconic acid, aldonic acid, uronic acid or ulosonic acid. In some embodiments, a linear monosaccharide can further include an amino sugar, such as glucosamine, N-acetyl glucosamine, galactosamine, and N-acetyl galactosamine.

Examples of PEG units having linear monosaccharides include the following:

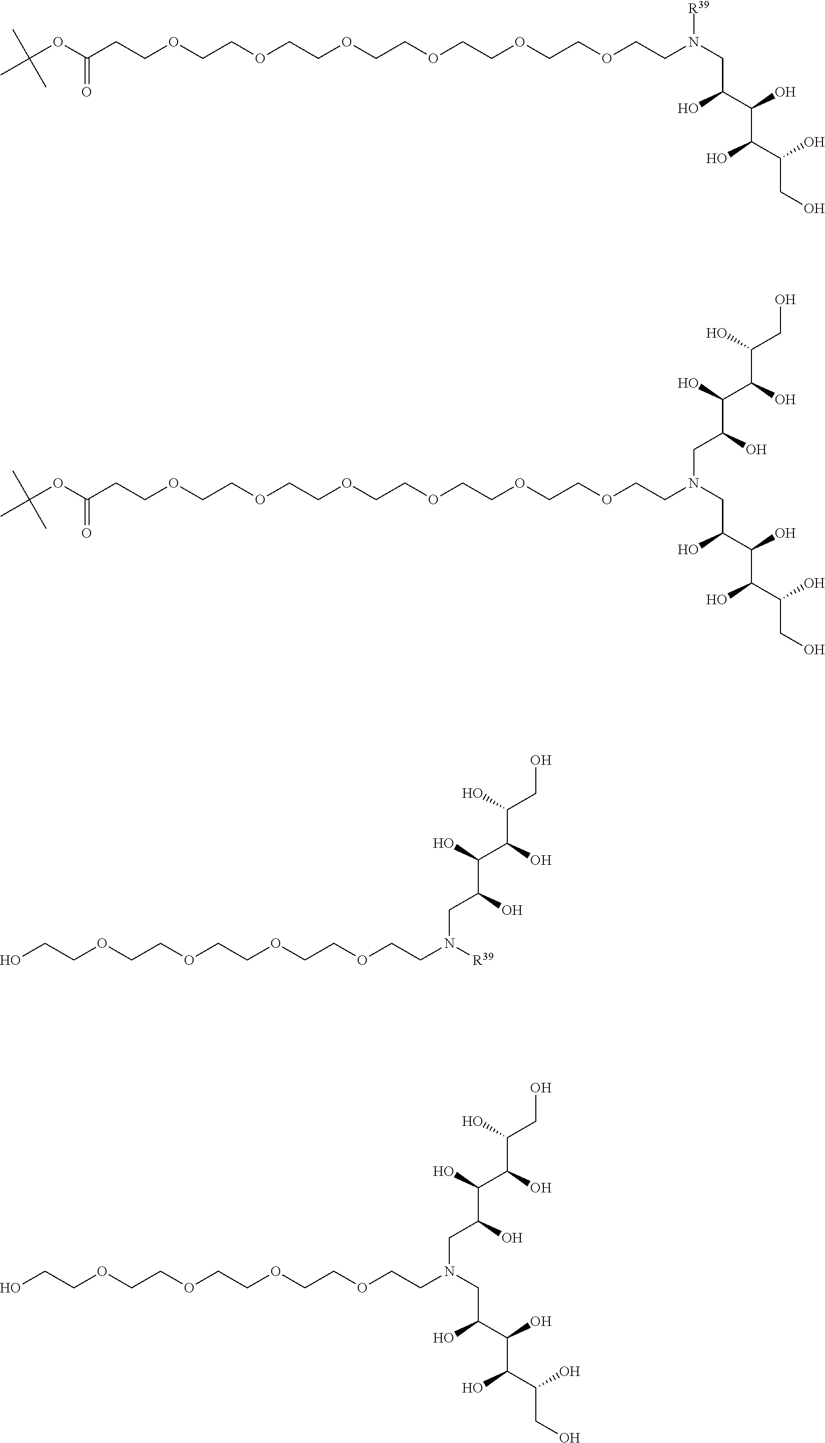

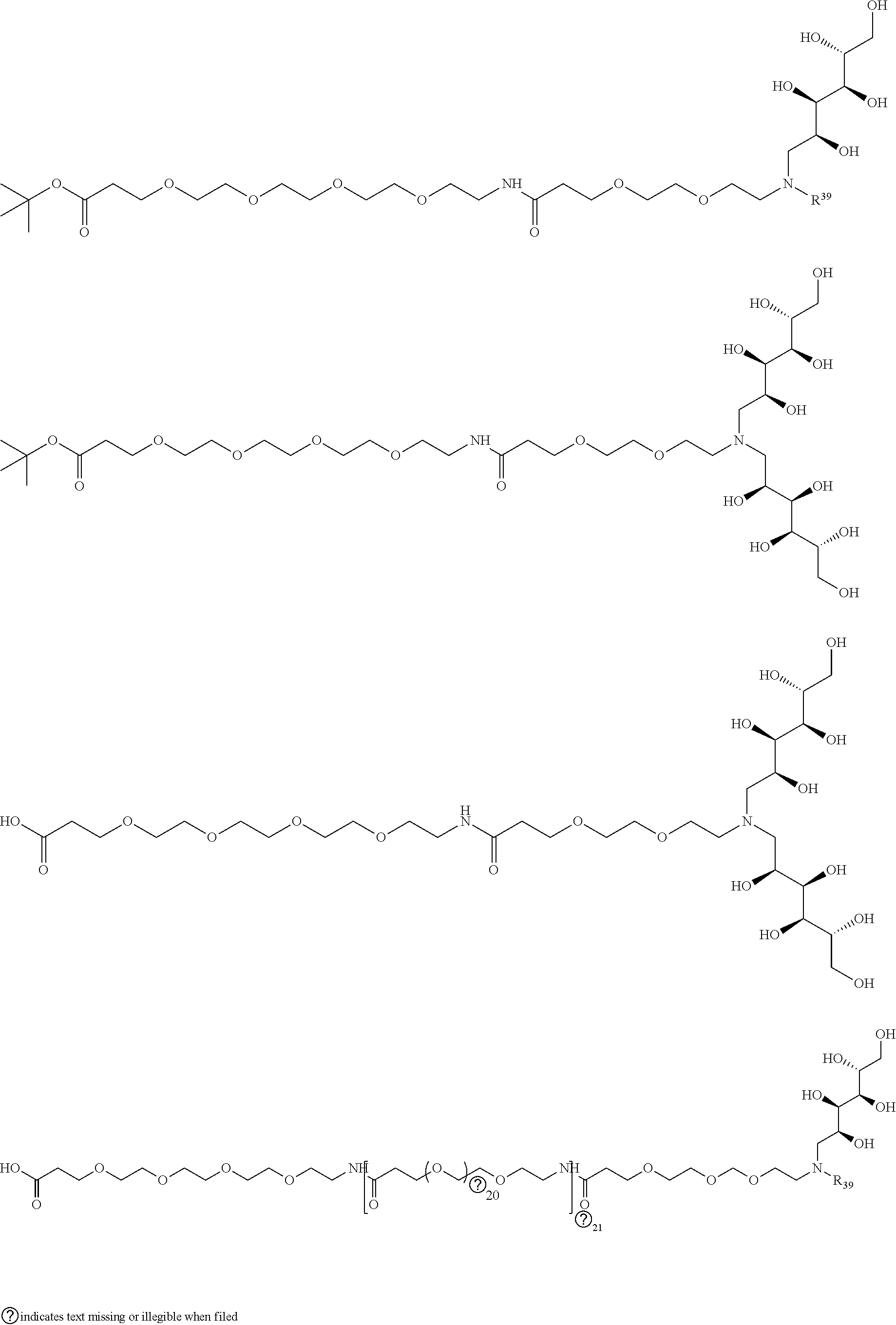

wherein R³⁹ is selected from H, a linear monosaccharide and polyethylene glycol. In these exemplary embodiments, when the PEG unit is attached to a subunit of an Amino Acid unit or to a portion of a Linker Subunit L2, it is deprotected, as needed, and a bond is formed between the carboxyl or hydroxyl group at the left end of the PEG unit and a reactive group on the subunit of an Amino Acid unit or portion of a Linker Subunit L2.

In some embodiments of a PEG unit of formula (XX) and (XXI), R²⁴ and R²⁵ are each independently selected from H and a polyhydroxyl group, provided that R²⁴ and R²⁵ are not both H. In some embodiments of a PEG unit of formula (XX) and (XXI), one of R²⁴ and R²⁵ is selected from a polyhydroxyl group and other is a polyethylene glycol. In some embodiments, each polyhydroxyl group includes at least three hydroxyl groups. A polyhydroxyl group can be linear or branched or cyclic. In some embodiments, one of R²⁴ and R²⁵ is a linear monosaccharide and the other is a cyclic monosaccharide. In some embodiments, one of R²⁴ and R²⁵ is a cyclic monosaccharide and the other is a linear or cyclic monosaccharide. In some embodiments, a linear monosaccharide is a linear (acyclic) form of a C6 or C5 sugar, such as glucose, ribose, galactose, mannose, arabinose, 2-deoxyglucose, glyceraldehyde, erythrose, threose, xylose, lyxose, allose, altrose, gulose, idose talose, aldose, and ketose. In some embodiments, a linear monosaccharide can further include a sugar acid, such as gluconic acid, aldonic acid, uronic acid or ulosonic acid. In some embodiments, a linear monosaccharide can further include an amino sugar, such as glucosamine, N-acetyl glucosamine, galactosamine, and N-acetyl galactosamine. In some embodiments, a cyclic monosaccharide is a cyclic form of a C6 or C5 sugar, such as glucose, ribose, galactose, mannose, arabinose, 2-deoxyglucose, glyceraldehyde, erythrose, threose, xylose, lyxose, allose, altrose, gulose, idose, talose, aldose, and ketose. In some embodiments, a cyclic monosaccharide can further include a sugar acid, such as gluconic acid, aldonic acid, uronic acid or ulosonic acid. In some embodiments, a cyclic monosaccharide can further include an amino sugar, such as glucosamine, N-acetyl glucosamine, galactosamine, and N-acetyl galactosamine. The stereochemistry at the anomeric C-1 position can be either alpha or beta.

Examples of PEG units include the following:

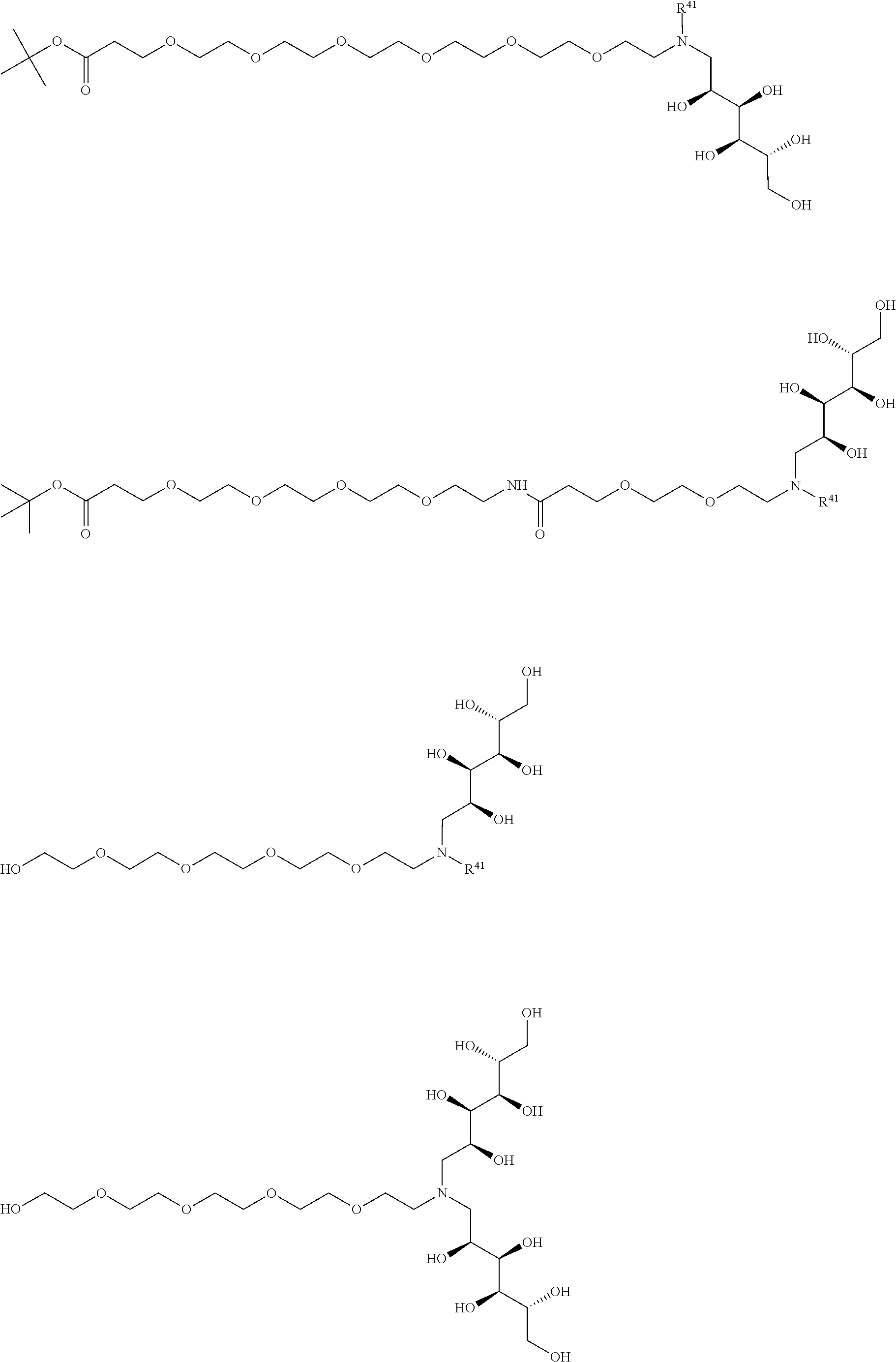

wherein R⁴¹ is a linear monosaccharide, a cyclic monosaccharide or polyethylene glycol. In these exemplary embodiments, when the PEG unit is attached to a subunit of an Amino Acid unit or to a portion of a Linker Subunit L2, it is deprotected, as needed, and a bond formed between the carboxyl or hydroxyl group at the left end of the PEG unit and a reactive group on the subunit of an Amino Acid unit or portion of a Linker Subunit L2.

In some embodiments of a PEG unit of formula (XX) and (XXI), R²⁴ and R²⁵ are each independently selected from H and polyhydroxyl group, provided that R²⁴ and R²⁵ are not both H. In some embodiments, each of R²⁴ and R²⁵ is a cyclic monosaccharide, disaccharide or polysaccharide. In some embodiments of a PEG unit of formula (XX) and (XXI), one of R²⁴ and R²⁵ is selected from a cyclic monosaccharide, disaccharide or polysaccharide and the other of R²⁴ and R²⁵ is polyethylene glycol. In some embodiments, a cyclic monosaccharide is a cyclic form of a C6 or C5 sugar, such as glucose, ribose, galactose, mannose, arabinose, 2-deoxyglucose, glyceraldehyde, erythrose, threose, xylose, lyxose, allose, altrose, gulose, idose, talose, aldose, and ketose. In some embodiments, a cyclic monosaccharide can further include a sugar acid, such as gluconic acid, aldonic acid, uronic acid or ulosonic acid. In some embodiments, a cyclic monosaccharide can further include an amino sugar, such as glucosamine, N-acetyl glucosamine, galactosamine, and N-acetyl galactosamine. The stereochemistry at the anomeric C-1 position can be either alpha or beta.

In some embodiments, a disaccharide includes those containing any of the monosaccharides described above. The term disaccharide can include linear forms, cyclic forms and linear-cyclic forms of a disaccharide. Exemplary disaccharides include, but are not limited to, sucrose, lactose, maltose, trehalose, and cellobiose. In some embodiments, a polysaccharide includes those containing any of the monosaccharides described above. The term polysaccharide can include linear forms, cyclic forms and linear-cyclic forms of a polysaccharide. Exemplary polysaccharides include, but are not limited to, maltotriose, raffinose, kestose, starch, cellulose, and glycogen.

In exemplary embodiments, PEG units with cyclic monosaccharide, disaccharide or polysaccharides include the following:

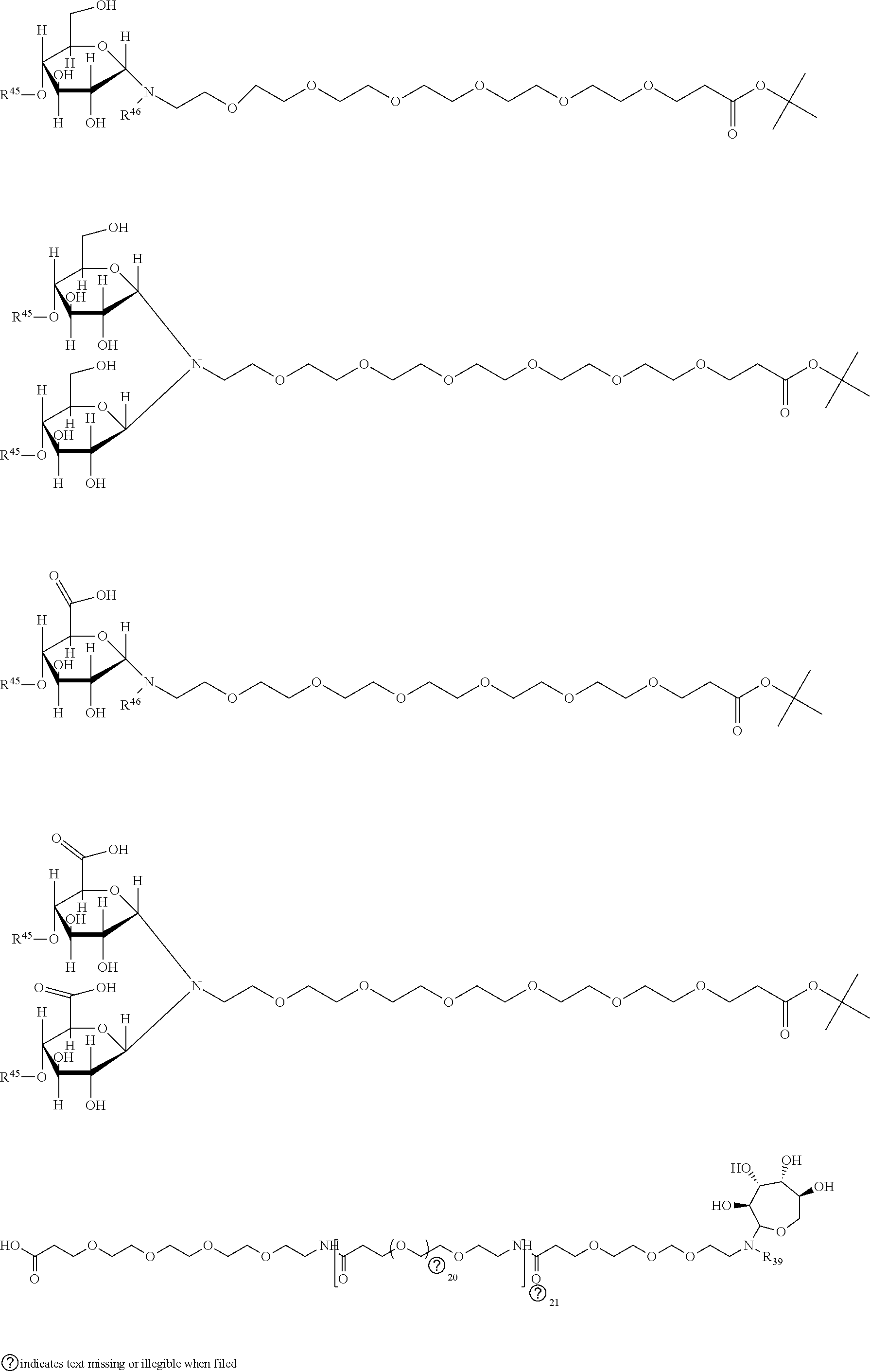

In each of these examples, each R⁴⁵ is selected from H, or a monosaccharide, a disaccharide, or a polysaccharide, including amino sugars of any of these; and R⁴⁶— is selected from H, or a monosaccharide, a disaccharide, or a polysaccharide, including amino sugars of any of these, and polyethylene glycol. In these exemplary embodiments, when the PEG unit is attached to a subunit of an Amino Acid unit or a portion of a Linker Subunit L2, it is deprotected, as needed, and a bond is formed between the carboxyl group at the right end (first four examples) or left end (last example) of the PEG unit and a reactive group on the subunit of an Amino Acid unit or portion of a Linker Subunit L2.

In some embodiments of a PEG unit of formula (XX) and (XXI), R²⁴ and R²⁵ are each independently selected from a polyhydroxyl group that is a linear monosaccharide or a substituted linear monosaccharide. In some embodiments of a PEG unit of formula (XX) and (XXI), one of R²⁴ and R²⁵ is selected from a polyhydroxyl group that is a linear monosaccharide or a substituted linear monosaccharide and the other of R²⁴ and R²⁵ is a polyethylene glycol In some embodiments, a linear monosaccharide is a linear form of a C6 or C5 sugar, such as glucose, ribose, galactose, mannose, arabinose, 2-deoxyglucose, glyceraldehyde, erythrose, threose, xylose, lyxose, allose, altrose, gulose, idose talose, aldose, and ketose. In some embodiments, a linear monosaccharide can further include a sugar acid, such as gluconic acid, aldonic acid, uronic acid or ulosonic acid. In some embodiments, a linear monosaccharide can further include an amino sugar, such as glucosamine, N-acetyl glucosamine, galactosamine, and N-acetyl galactosamine.

In some embodiments, the substituted linear monosaccharide can be substituted with a monosaccharide, a disaccharide or a polysaccharide, in each case either linear or cyclic. In some embodiments, a disaccharide includes those containing any of the monosaccharides described above. The term disaccharide can include linear forms, cyclic forms and linear-cyclic forms of a disaccharide. Exemplary disaccharides include, but are not limited to, sucrose, lactose, maltose, trehalose, and cellobiose. In some embodiments, a polysaccharide include those containing any of the monosaccharides described above. The term polysaccharide can include linear forms, cyclic forms and linear-cyclic forms of a polysaccharide. Exemplary polysaccharides include, but are not limited to, maltotriose, raffinose, kestose, starch, cellulose, and glycogen.

Examples of PEG units containing linear monosaccharides optionally substituted with saccharides include the following:

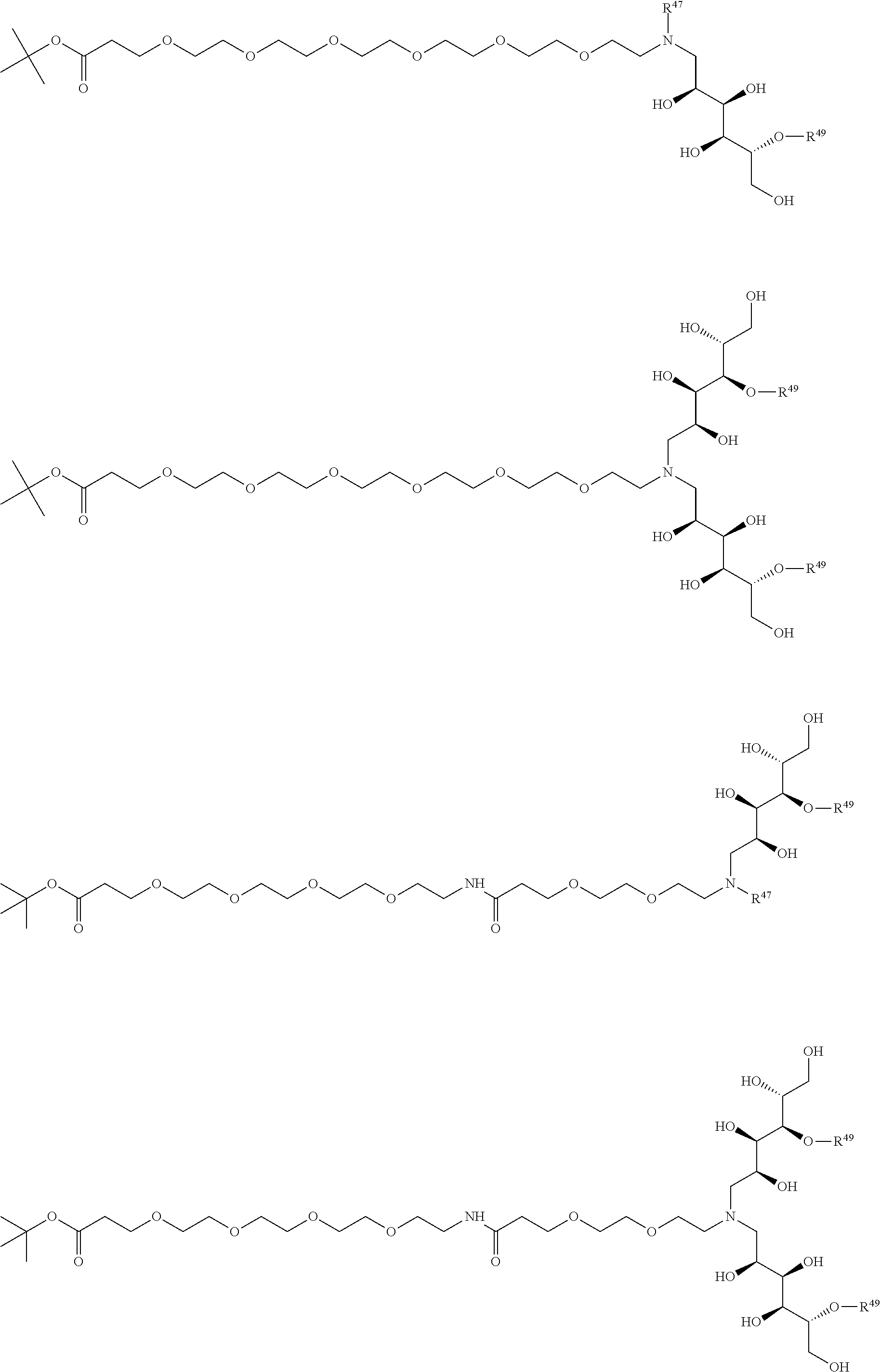

wherein R⁴⁷ is selected from H, a linear monosaccharide, and polyethylene glycol; and each R⁴⁹ is selected from a monosaccharide, a disaccharide and a polysaccharide. In these exemplary embodiments, when the PEG unit is attached to a subunit of an Amino Acid unit or a portion of a Linker Subunit L2, it is deprotected, as needed, and a bond is formed between the carboxyl or hydroxyl group at the left end of the PEG unit and a reactive group on the subunit of an Amino Acid unit or portion of a Linker Subunit L2.

In some embodiments of a PEG unit of formula (XX) and (XXI), R²⁴ and R²⁵ are each independently selected from a polyhydroxyl group that is a linear monosaccharide or a substituted linear monosaccharide, wherein the substituted linear monosaccharide is substituted with one or more substituents such as alkyl, O-alkyl, aryl, O-aryl, carboxyl, ester, or amide. In some embodiments of a PEG unit of formula (XX) and (XXI), one of R²⁴ and R²⁵ is selected from a polyhydroxyl group that is a linear monosaccharide or a substituted linear monosaccharide, wherein the substituted linear monosaccharide is substituted with one or more substituents such as alkyl, O-alkyl, aryl, O-aryl, carboxyl, ester, or amide; and the other of R²⁴ and R²⁵ is polyethylene glycol. Such a substituted polyhydroxyl group can be optionally further substituted with a monosaccharide, disaccharide or polysaccharide.

In exemplary embodiments, PEG units having a polyhydroxyl group comprising linear monosaccharide or a substituted linear monosaccharide include the following:

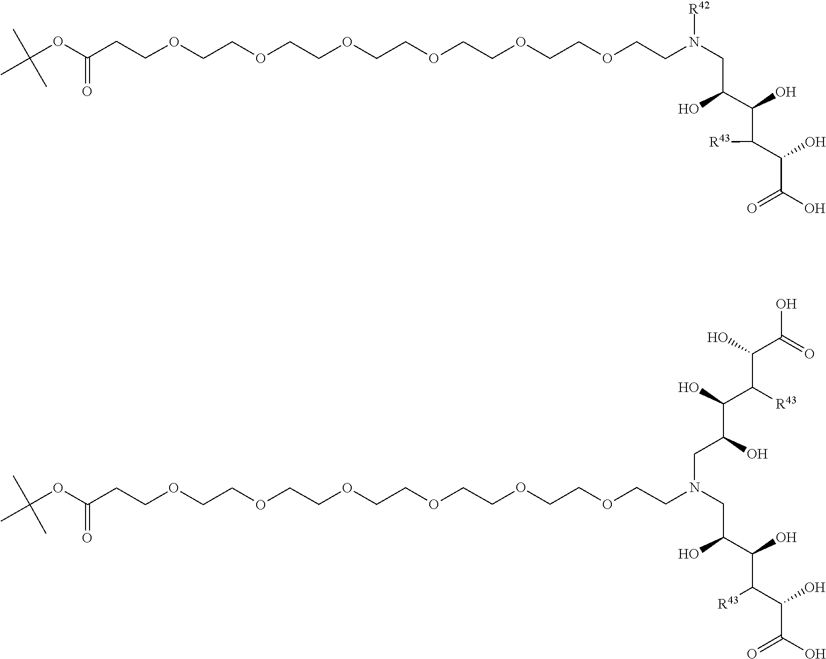

In each of these examples, each R⁴² is independently selected from H, a monosaccharide, a disaccharide or a polysaccharide, as described herein, or polyethylene glycol; and each R⁴³ is selected from alkyl, O-alkyl, aryl, O-aryl, carboxyl, ester, or amide. In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XX) and (XXI), at least one of R²⁴ and R²⁵ is —C(O)-polyhydroxyl group or substituted —C(O)-polyhydroxyl group and the other of R²⁴ and R²⁵ is —C(O)-polyhydroxyl group; substituted —C(O)-polyhydroxyl group, polyhydroxyl group or substituted polyhydroxyl group. In some embodiments, the substituted —C(O)— polyhydroxyl group and polyhydroxyl group can be substituted with a monosaccharide, a disaccharide or a polysaccharide (in each case either linear or cyclic); alkyl; O-alkyl; aryl; carboxyl; ester; or amide. In some embodiments, a disaccharide includes those containing any of the monosaccharides described above. The term disaccharide can include linear forms, cyclic forms and linear-cyclic forms of a disaccharide. Exemplary disaccharides include, but are not limited to, sucrose, lactose, maltose, trehalose, and cellobiose. In some embodiments, a polysaccharide includes those containing any of the monosaccharides described above. The term polysaccharide can include linear forms, cyclic forms and linear-cyclic forms of a polysaccharide. Exemplary polysaccharides include, but are not limited to, maltotriose, raffinose, kestose, starch, cellulose, and glycogen.

In exemplary embodiments, PEG units having a —C(O)-polyhydroxyl group or substituted —C(O)-polyhydroxyl group include the following:

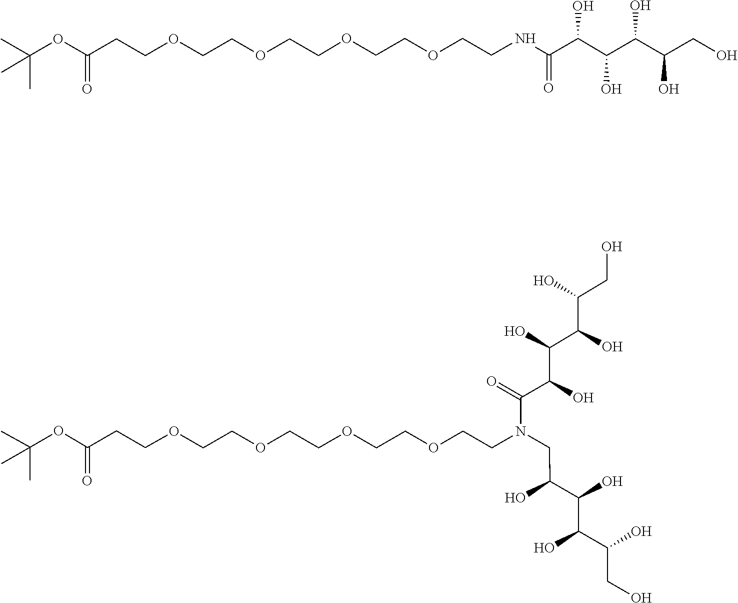 [SMILES: CC(C)(C)OC(=O)CCOCCOCCOCCOCCNC(=O)[C@H](O)[C@@H](O)[C@H](O)[C@H](O)CO ; CC(C)(C)OC(=O)CCOCCOCCOCCOCCN(C[C@H](O)[C@@H](O)[C@H](O)[C@H](O)CO)C(=O)[C@H](O)[C@@H](O)[C@H](O)[C@H](O)CO]

In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XX) and (XXI), R²⁴ and R²⁵ are independently selected from H and substituted —C₁-C₈ alkyl; provided that both R²⁴ and R²⁵ are not H. In some embodiments, R²⁴ and R²⁵ are independently selected from H and substituted —C₁-C₄ alkyl; provided that both R²⁴ and R²⁵ are not H. In some embodiments, R²⁴ and R²⁵ are independently selected from H and substituted —C₁-C₃ alkyl; provided that both R²⁴ and R²⁵ are not H. The alkyl portion of a substituted —C₁-C₈, —C₁-C₄, and —C₁-C₃ alkyl can be straight chain or branched.

Substituted —C₁-C₈, —C₁-C₄, or —C₁-C₃ alkyl can be substituted with hydroxyl or carboxyl. In some embodiments, each carbon atom of a substituted —C₁-C₈, —C₁-C₄, or —C₁-C₃ alkyl is substituted with hydroxyl or carboxyl. In some embodiments, each carbon atom of a substituted —C₁-C₈, —C₁-C₄, or —C₁-C₃ alkyl is substituted with carboxyl. In some embodiments, one or two carbon atoms of a substituted —C₁-C₈, —C₁-C₄, or —C₁-C₃ alkyl are substituted with hydroxyl or carboxyl. In some embodiments, one or two carbon atoms of a substituted —C₁-C₈, —C₁-C₄, or —C₁-C₃ alkyl are substituted with carboxyl. In some embodiments, the terminal carbon atom of a substituted —C₁-C₈, —C₁-C₄, or —C₁-C₃ alkyl is substituted with carboxyl. In some embodiments, the terminal carbon atom of a substituted -C₁-C₈, —C₁-C₄, or —C₁-C₃ alkyl is substituted with hydroxyl.

Exemplary embodiments of a PEG unit having a substituted —C₁-C₈, —C₁-C₄, or —C₁-C₃ alkyl are as follows:

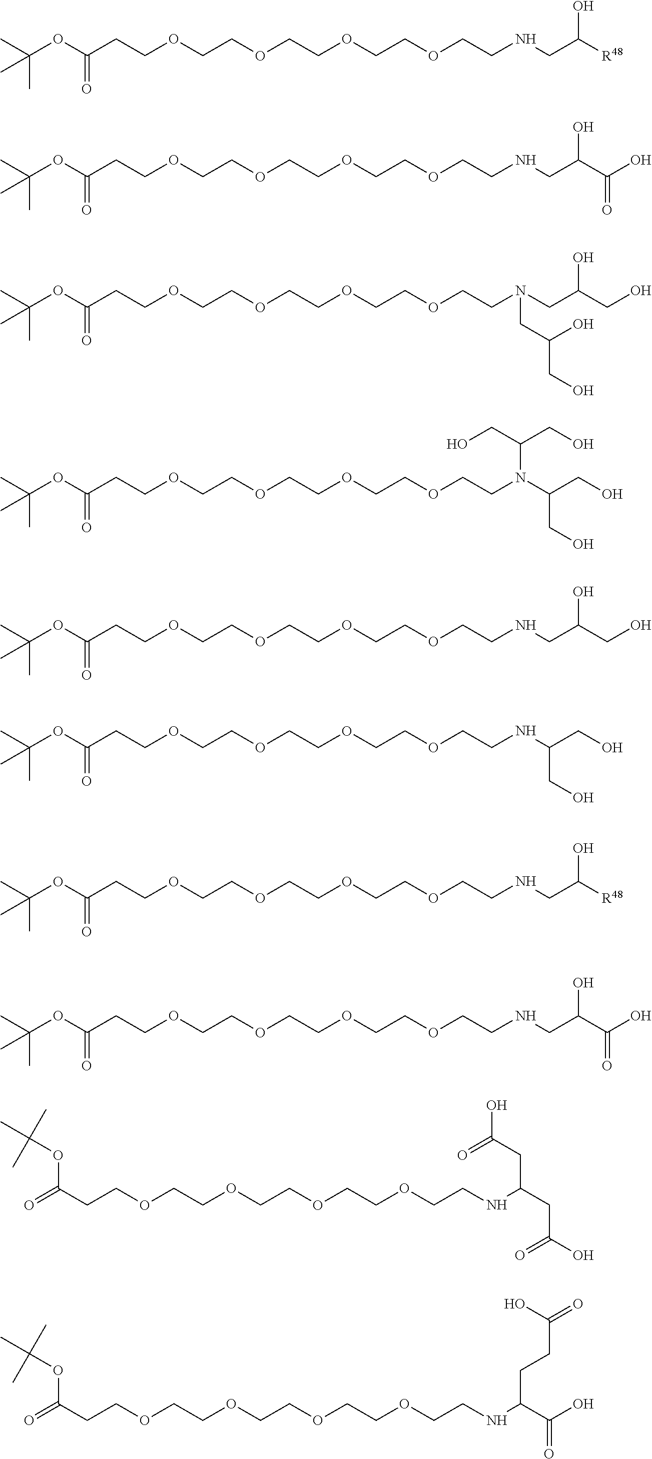

wherein R⁴⁸ can be H, OH, CH₂OH, COOH, or —C₁-C₆ alkyl substituted with hydroxyl and/or carboxyl. In these exemplary embodiments, when the PEG unit is attached to a subunit of an Amino Acid unit or a portion of a Linker Subunit L2, it is deprotected and a bond formed between the carboxyl group at the left end of the PEG unit and a reactive group on the subunit of an Amino Acid unit or portion of a Linker Subunit L2.

In some embodiments of a PEG unit of formula (XX) and (XXI), one of R²⁴ and R²⁵ are selected from H and substituted —C(O)—C₁-C₈ alkyl and the other of R²⁴ and R²⁵ is selected from substituted —C(O)—C₁-C₈ alkyl, —C(O)—C₁-C₄ alkyl, and —C(O)—C₁-C₃ alkyl and substituted —C₁-C₈ alkyl, —C₁-C₄ alkyl, and —C₁-C₃ alkyl (as described above). In some embodiments, one of R²⁴ and R²⁵ are independently selected from H and substituted —C(O)—C₁-C₄ alkyl and the other of R²⁴ and R²⁵ is selected from substituted —C(O)—C₁-C₈ alkyl, —C(O)—C₁-C₄ alkyl, and —C(O)—C₁-C₃ alkyl and substituted —C₁-C₈ alkyl, —C₁-C₄ alkyl, and —C₁-C₃ alkyl (as described above). In some embodiments, one of R²⁴ and R²⁵ are selected from H and substituted —C(O)—C₁-C₃ alkyl and the other of R²⁴ and R²⁵ is selected from substituted —C(O)—C₁-C₈ alkyl, —C(O)—C₁-C₄ alkyl, and —C(O)—C₁-C₃ alkyl and substituted —C₁-C₈ alkyl, —C₁-C₄ alkyl, and —C₁-C₃ alkyl (as described above). The alkyl of substituted —C(O)—C₁-C₈ alkyl, —C(O)—C₁-C₈ alkyl, and —C(O)—C₁-C₈ alkyl can be straight chain or branched. The alkyl portion of a substituted —C₁-C₈, —C₁-C₄, and —C₁-C₃ alkyl can be straight chain or branched.

Substituted —C(O)—C₁-C₈ alkyl, —C(O)—C₁-C₄ alkyl, and —C(O)—C₁-C₃ alkyl can be substituted with hydroxyl or carboxyl. In some embodiments, each carbon atoms of a substituted —C(O)—C₁-C₈ alkyl, —C(O)—C₁-C₄ alkyl, and —C(O)—C₁-C₄ alkyl is substituted with hydroxyl or carboxyl. In some embodiments, one or two carbon atoms of a substituted —C(O)—C₁-C₈ alkyl, —C(O)—C₁-C₄ alkyl, and —C(O)—C₁-C₃ alkyl is substituted with hydroxyl or carboxyl. In some embodiments, one or two carbon atoms of a substituted —C(O)—C₁-C₈ alkyl, —C(O)—C₁-C₄ alkyl, and —C(O)—C₁-C₃ alkyl are substituted with carboxyl. In some embodiments, the terminal carbon atom of a substituted —C(O)—C₁-C₈ alkyl, —C(O)—C₁-C₄ alkyl, and —C(O)—C₁-C₃ alkyl is substituted with carboxyl. In some embodiments, the terminal carbon atom of a substituted —C(O)—C₁-C₈ alkyl, —C(O)—C₁-C₄ alkyl, and —C(O)—C₁-C₃ alkyl is substituted with hydroxyl.

Exemplary embodiments of such PEG units include the following:

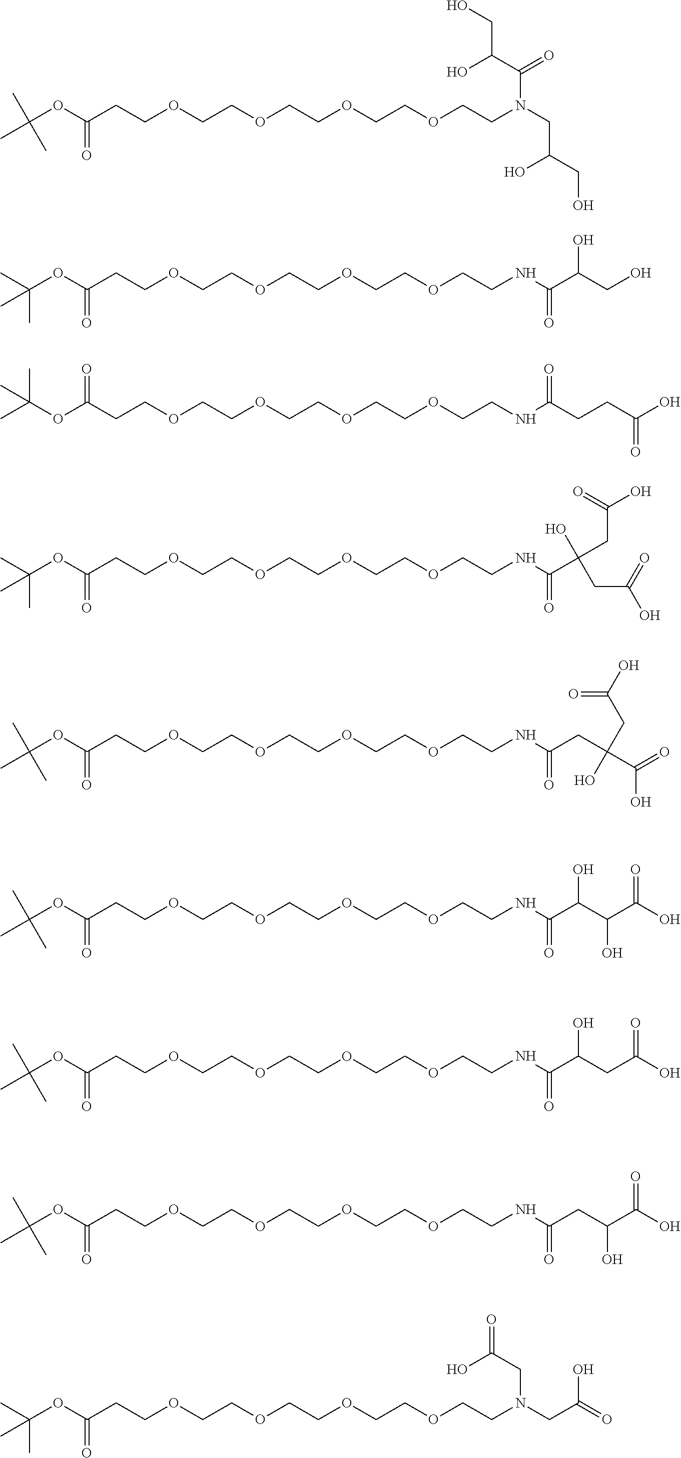

In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XX) and (XXI), R²⁴ and R²⁵ are selected from H and optionally substituted aryl; provided that both R²⁴ and R²⁵ are not H. In some embodiments, substituted aryl includes aryl substituted with halogen (such as chloro, fluoro and bromo).

In an exemplary embodiment, a PEG unit comprising a substituted aryl includes the following:

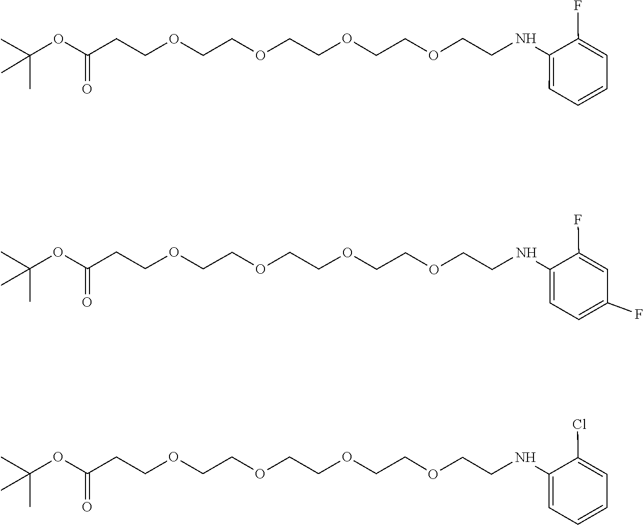 [SMILES: CC(C)(C)OC(=O)CCOCCOCCOCCOCCNc1ccccc1F ; CC(C)(C)OC(=O)CCOCCOCCOCCOCCNc1ccc(F)cc1F ; CC(C)(C)OC(=O)CCOCCOCCOCCOCCNc1ccccc1Cl]

In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XX) and (XXI), R²⁴ and R²⁵ together form an optionally substituted C₃-C₈ heterocycle or heteroaryl. In some embodiments, optional substituents include heterocycle or aryl substituted with halogen (such as chloro, fluoro and bromo).

In an exemplary embodiment, a PEG unit comprising an optionally substituted C₃-C₈ heterocycle includes the following:

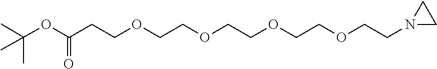 [SMILES: CC(C)(C)OC(=O)CCOCCOCCOCCOCCN1CC1]

In this exemplary embodiment, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XX) and (XXI), R²⁴ and R²⁵ are independently selected from H and a chelator; provided that both R²⁴ and R²⁵ are not H. In some embodiments, the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-,N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine. In some embodiments, a chelator is directly attached to the nitrogen of —NR²⁴R²⁵. In some embodiments, the chelator is attached via an alkylene, arylene, carbocyclo, heteroarylene or heterocarbocylo (in each case either substituted or unsubstituted).

In some exemplary embodiments, a PEG unit comprising a chelator includes the following:

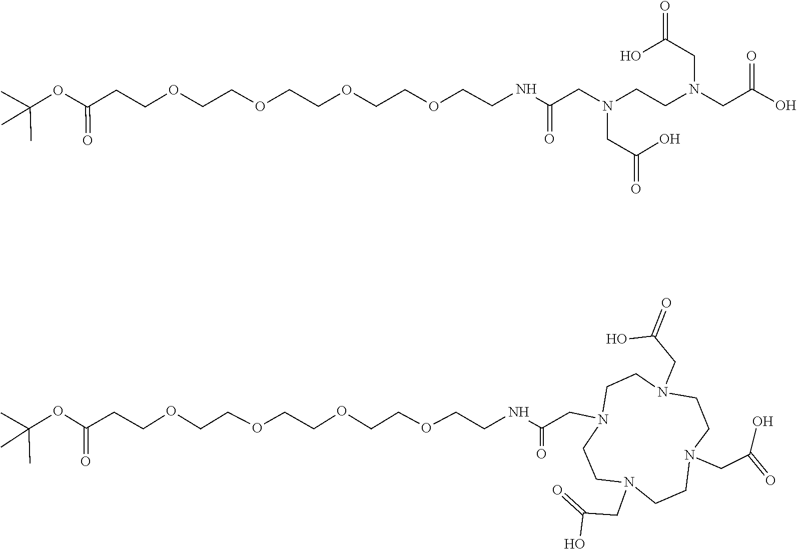 [SMILES: CC(C)(C)OC(=O)CCOCCOCCOCCOCCNC(=O)CN(CCN(CC(=O)O)CC(=O)O)CC(=O)O ; CC(C)(C)OC(=O)CCOCCOCCOCCOCCNC(=O)CN1CCN(CC(=O)O)CCN(CC(=O)O)CCN(CC(=O)O)CC1]

In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XX), (XXI), (XXX), (XXXI), (XXXII) and (XXXIII), a chelator can be appended to any of the R²⁴, R²⁵ and/or R³⁰ groups described herein. In some embodiments, the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-,N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,1 1-tetraacetic acid (TETA) and N,N′-dialkyl substituted piperazine. In some embodiments, a chelator is directly attached to the R²⁴, R²⁵ or R³⁰ groups described herein. In some embodiments, the chelator is attached via an alkylene, arylene, carbocycle, heteroaryl or heterocarbocyle (in each case either substituted or unsubstituted).

In some embodiments, a PEG unit has the following general formula (XXX):

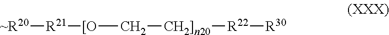

wherein R²⁰ is a functional group for attachment to a subunit of an Amino Acid unit and/or a portion of a Linker Subunit L2; R²¹ and R²² are each optional C₁-C₃ alkylene groups; R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; the wavy line (˜) indicates an attachment site; and n20 is 1 to 26. In some embodiments, R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof. Suitable protecting groups include carboxylic acid protecting groups, amine protecting groups, and sulfonyl protecting groups typically used in the art.

In some embodiments, a PEG unit has the following general formula (XXX):

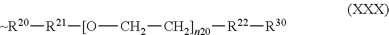

wherein R²⁰ is a functional group for attachment to a subunit of an Amino Acid unit and/or a portion of a Linker Subunit L2; R²¹ and R²² are each optional C₁-C₃ alkylene groups; R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; the wavy line (˜) indicates an attachment site; and n20 is 1 to 26. In some embodiments, R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, or protected forms thereof. Suitable protecting groups include carboxylic acid protecting groups, amine protecting groups, and sulfonyl protecting groups typically used in the art.

In some embodiments of a PEG unit of formula (XXX), R³⁰ is an optionally substituted C₃-C₁₀ carbocycle. In some embodiments, an optionally substituted C₃-C₁₀ carbocycle is a fused cyclooctyne compound as disclosed in International Publication Number WO 2011/136645 (the disclosure of which is incorporated by reference herein). Exemplary PEG units with a fused cyclooctyne are shown below.

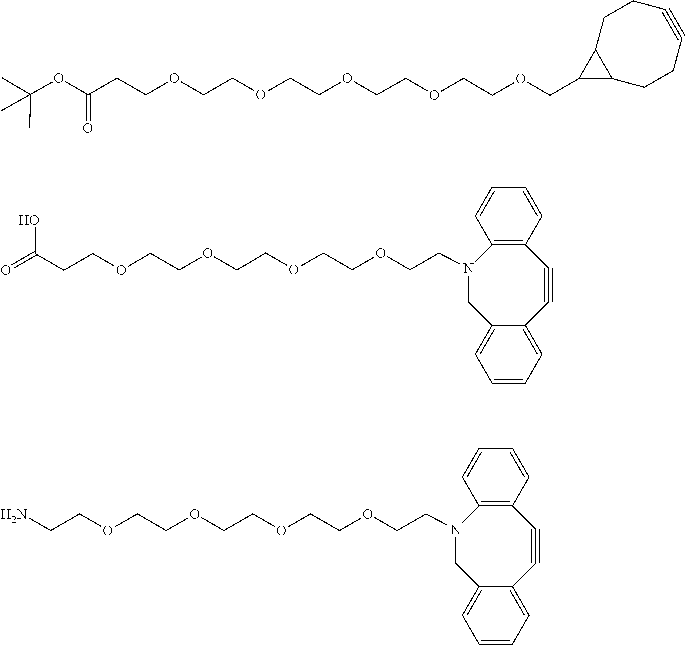 [SMILES: CC(C)(C)OC(=O)CCOCCOCCOCCOCCOCC1C2CCC#CCCC21 ; O=C(O)CCOCCOCCOCCOCCN1Cc2ccccc2C#Cc2ccccc21 ; NCCOCCOCCOCCOCCN1Cc2ccccc2C#Cc2ccccc21]

In these exemplary embodiments, when the PEG unit is attached to a subunit of an Amino Acid unit or a portion of a Linker Subunit L2, it is deprotected, as needed, and a bond is formed between the carboxyl or amino group at the left end of the PEG unit and a reactive group on the subunit of an Amino Acid unit or portion of a Linker Subunit L2.

As will be appreciated by persons of skill in the art, the above compound as well as others disclosed in International Publication Number WO 2011/136645 can be used as an intermediate for click chemistry for the attachment of additional compounds. In some embodiments, the additional compound is a Drug unit. In some embodiments, the additional compound is a Linker Subunit L2 as described herein.

In some embodiments of a PEG unit of formula (XXX), R³⁰ is a thiourea; a substituted thiourea, a urea or a substituted urea. A thiourea and urea group can be substituted with, for example, optionally substituted alkyl, optionally substituted carbocycle, or optionally substituted aryl.

Exemplary PEG units comprising a thiourea; a substituted thiourea; a urea; or a substituted urea, include the following:

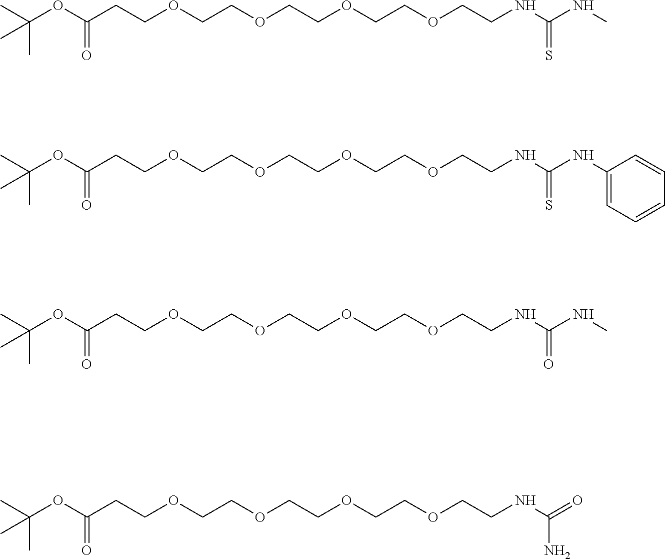

In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XXX), R³⁰ is a sulfamide; alkyl sulfamide; acyl sulfamide, optionally substituted alkyl sulfamide; optionally substituted acyl sulfamide; sulfonamide; or optionally substituted sulfonamide. Optionally substituted alkyl sulfamide; optionally substituted acyl sulfamide; and optionally substituted sulfonamide can be substituted with groups to increase solubility or, in other embodiments, for attachment of additional groups, such as linkers, Drugs or other Compounds.

Exemplary PEG units comprising a sulfamide; alkyl sulfamide; acyl sulfamide, optionally substituted alkyl sulfamide; optionally substituted acyl sulfamide; sulfonamide; or optionally substituted sulfonamide, include the following:

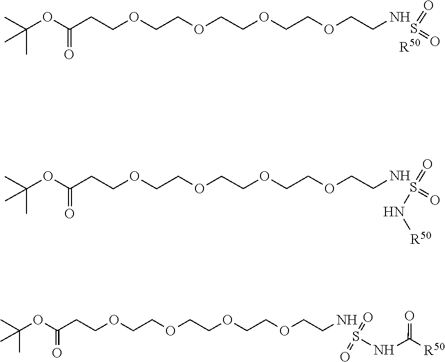

In these examples, R⁵⁰ can be, for example, optionally substituted alkyl, alkenyl, alkynyl, carbocycle, aryl, heterocarbocycle or heteroaryl. In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XXX), R³⁰ is a guanidine or an optionally substituted guanidine. Optionally substituted guanidine can be substituted with optionally substituted alkyl, alkenyl, alkynyl, carbocycle, aryl, heterocarbocycle or heteroaryl.

Exemplary PEG units comprising a guanidine or optionally substituted guanidine include the following:

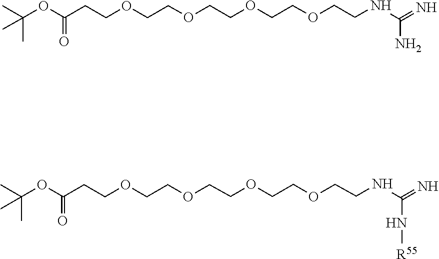

In these examples, R⁵⁵ can be, for example, optionally substituted alkyl, alkenyl, alkynyl, carbocycle, aryl, heterocarbocycle or heteroaryl. In these exemplary embodiments, when the PEG unit is attached to a subunit of an Amino Acid unit or a portion of a Linker Subunit L2, it is deprotected, as needed, and a bond is formed between the carboxyl group at the left end of the PEG unit and a reactive group on the subunit of an Amino Acid unit or portion of a Linker Subunit L2.

In some embodiments of a PEG unit of formula (XXX), R³⁰ is a phosphoramide or an optionally substituted phosphoramide. Optionally substituted phosphoramide can be substituted with optionally substituted alkyl, alkenyl, alkynyl, carbocycle, aryl, heterocarbocycle or heteroaryl.

Exemplary PEG units comprising a phosphoramide or optionally substituted phosphoramide include the following:

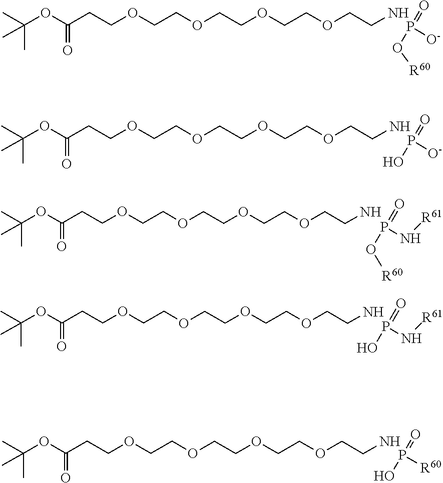

In these examples, R⁶⁰ can be, for example, optionally substituted alkyl, alkenyl, alkynyl, carbocycle, aryl, heterocarbocycle or heteroaryl. R⁶¹ can be, for example, optionally substituted alkyl, alkenyl, alkynyl, carbocycle, aryl, heterocarbocycle or heteroaryl. In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments of a PEG unit of formula (XXX), a PEG unit comprises a functional group for attachment of additional moieties. In some embodiments of a PEG unit of formula (XXX), R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl. In some embodiments, such a PEG unit can be used as an intermediate for click chemistry for the attachment of additional compounds. In some embodiments, the additional compound is a Drug unit. In some embodiments, the additional compound is a Linker Subunit L2 as described herein. In some embodiments, the additional compound is another linker or a drug linker.

Exemplary PEG units comprising an azido, alkynyl or cyclooctyne group include the following:

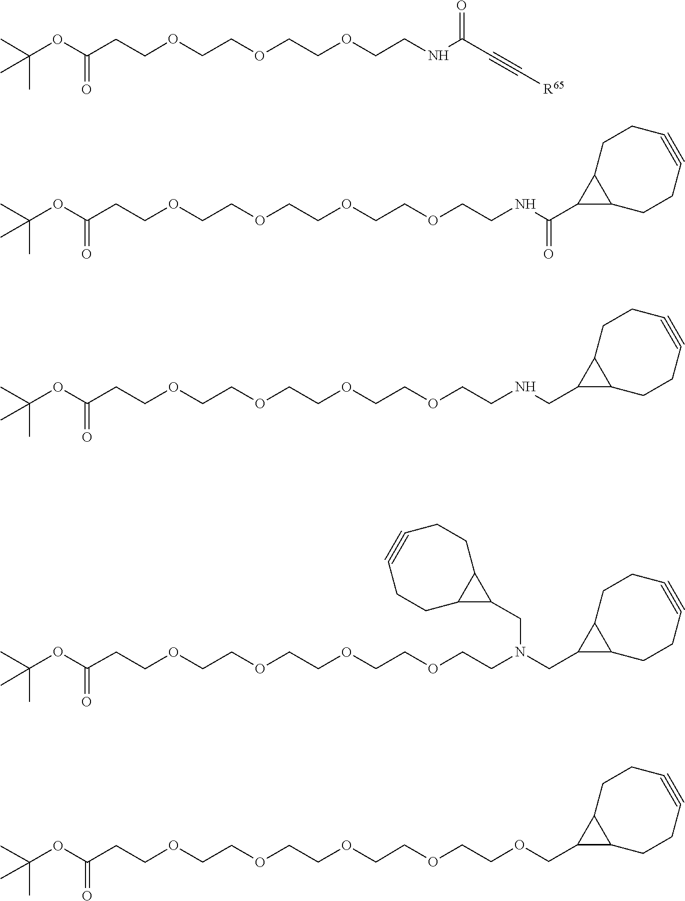

In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments a PEG unit has the following formula:

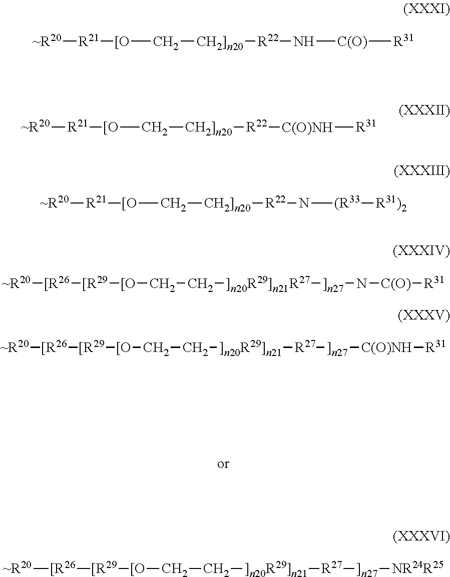

or a salt thereof, wherein R²⁰ is a functional group for attachment to a subunit of an Amino Acid unit or a portion of a Linker Subunit L2; R²¹ and R²² are each optional C₁-C₃ alkylene groups; R²⁶ and R²⁷ are each optional C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— or —C(O)—C₁-C₁₂ alkylene-NH—; R³¹ is a branched polyethylene glycol chain, each branch having 1 to 26 ethylene glycol subunits and each branch having an R³⁵ at its terminus; R³³ is C₁-C₃ alkylene, C₁-C₃ alkylene-C(O), —C(O)—C₁-C₃ alkylene, or —C(O)—C₁-C₃ alkylene-C(O); each R²⁹ is optional and independently selected from —C(O)—, —NH—, —C(O)—C₁-C₆ alkenylene-, —NH—C₁-C₆alkenylene-, —C₁-C₆ alkenylene-NH— and —C₁-C₆ alkenylene-C(O)—; R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy line (˜) indicates an attachment site; n20 is 1 to 26; n21 is 1 to 4; and n27 is 1 to 4;. In some embodiments, R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof. Suitable protecting groups include carboxylic acid protecting groups, amine protecting groups, and sulfonyl protecting groups typically used in the art.

In some embodiments a PEG unit has the following formula:

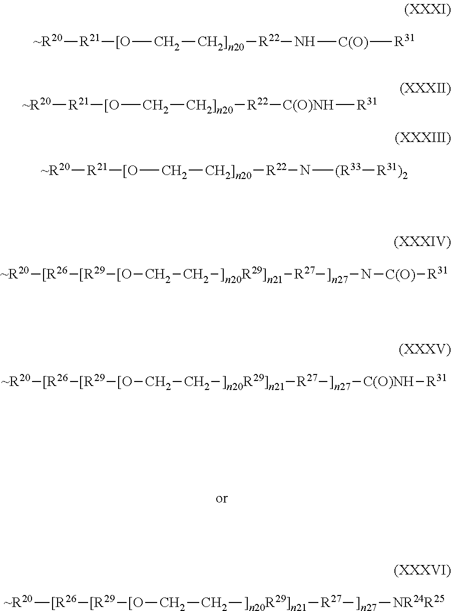

or a salt thereof, wherein R²⁰ is a functional group for attachment to a subunit of an Amino Acid unit or a portion of a Linker Subunit L2; R²¹ and R²² are each optional C₁-C₃ alkylene groups; R²⁶ and R²⁷ are each optional C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— or —C(O)—C₁-C₁₂ alkylene-NH—; R³¹ is a branched polyethylene glycol chain, each branch having 1 to 26 ethylene glycol subunits and each branch having an R³⁵ at its terminus; R³³ is C₁-C₃ alkylene, C₁-C₃ alkylene-C(O), —C(O)—C₁-C₃ alkylene, or —C(O)—C₁-C₃ alkylene-C(O); each R²⁹ is optional and independently selected from —C(O)—, —NH—, —C(O)—C₁-C₆ alkenylene-, —NH—C₁-C₆ alkenylene-, —C₁-C₆ alkenylene-NH—, —C₁-C₆ alkenylene-C(O)—, —NH(CO)NH—, and triazole;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy line (˜) indicates an attachment site; n20 is 1 to 26; n21 is 1 to 4; and n27 is 1 to 4;. In some embodiments, R²⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate triazole, azadibenzocyclooctyne, hydrazine, carbonylalkylheteroaryl, or protected forms thereof. Suitable protecting groups include carboxylic acid protecting groups, amine protecting groups, and sulfonyl protecting groups typically used in the art.

As will be appreciated by those skilled in the art, such PEG units can be used for the attachment of additional compounds. In some embodiments, the additional compound is a Drug unit. In some embodiments, the additional compound is a Linker Subunit L2 as described herein. In some embodiments, the additional compound is a linker or a drug linker.

Exemplary PEG units comprising branched polyethylene glycol chain include the following:

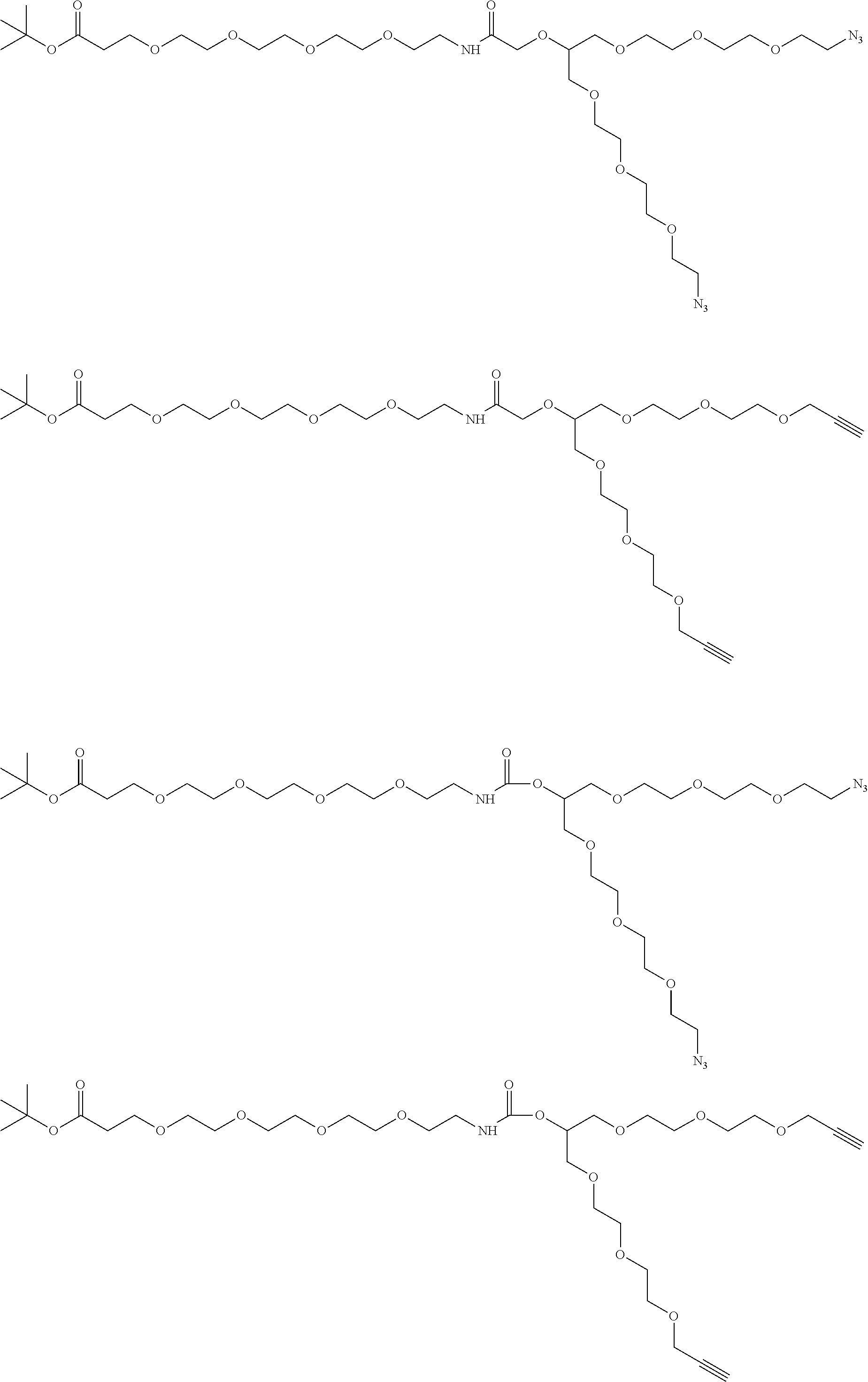

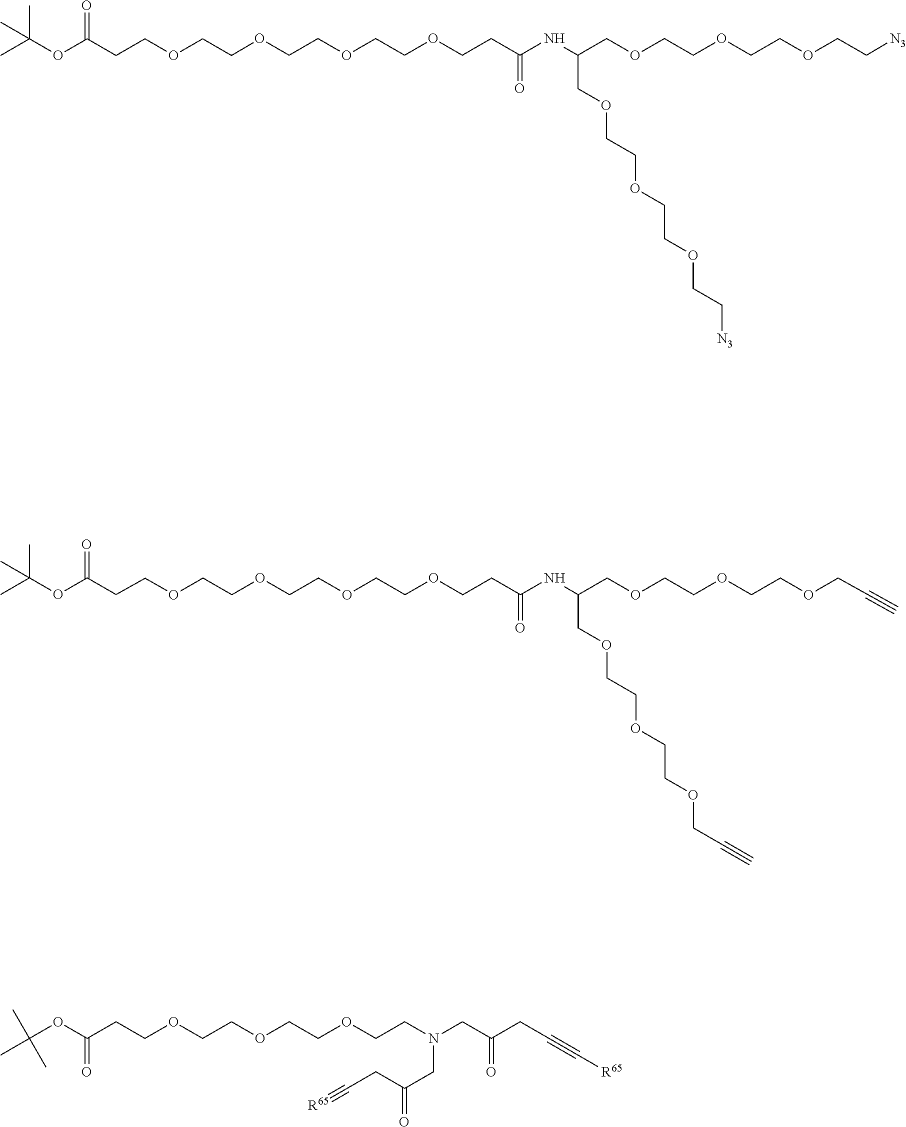

In these exemplary embodiments, when the PEG unit is attached to a subunit of an Amino Acid unit or a portion of a Linker Subunit L2, it is deprotected and a bond is formed between the carboxyl group at the left end of the PEG unit and a reactive group on the subunit of an Amino Acid unit or portion of a Linker Subunit L2.

In some embodiments, provided is a Linker intermediate or Linker, comprising a PEG unit having a formula selected from:

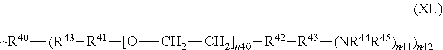

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₆ alkylene;
each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, a polyhydroxyl group, a substituted polyhydroxyl group, a —C(O)-polyhydroxyl group, or a substituted —C(O)— polyhydroxyl group, wherein optional substituents are selected from sulfate, phosphate, alkyl sulfate, and alkyl phosphate;
the wavy line (˜) indicates the attachment site to R⁴⁰;
n40 is 1 to 26;
n41 is 1 to 6; and
n42 is 1 to 6.

In some embodiments, provided is a Linker intermediate or Linker, comprising a PEG unit having a formula selected from:

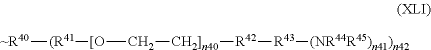

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₆ alkylene;
R⁴³ is absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)— polyhydroxyl group, wherein optional substituents are selected from sulfate, phosphate, alkyl sulfate, and alkyl phosphate;
the wavy line (˜) indicates the attachment site to R⁴⁰;
n40 is 1 to 26;
n41 is 1 to 6; and
n42 is 1 to 6.

In some embodiments, provided is a Linker intermediate or Linker, comprising a PEG unit having a formula selected from:

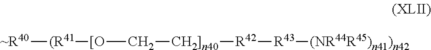

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₃ alkylene;
R⁴³ is absent or is selected from selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
R⁴⁴ and R⁴⁵ are each, independently, H, a polyhydroxyl group, a substituted polyhydroxyl group, a —C(O)-polyhydroxyl group, or a substituted —C(O)— polyhydroxyl group, wherein optional substituents are selected from sulfate, phosphate, alkyl sulfate, and alkyl phosphate;
the wavy line (˜) indicates the attachment site to R⁴⁰;
n40 is 1 to 16;
n41 is 1 to 4; and
n42 is 1 to 4.

In some embodiments, provided is a Linker intermediate or Linker, wherein R⁴⁰ is selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

In some embodiments, provided is a Linker intermediate or Linker, wherein R⁴⁰ has one of the following structures:

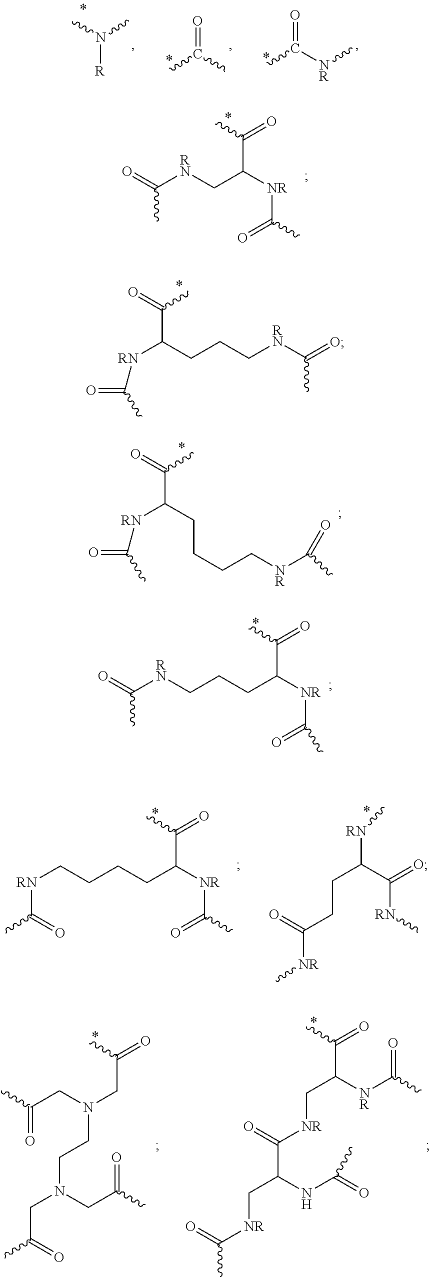

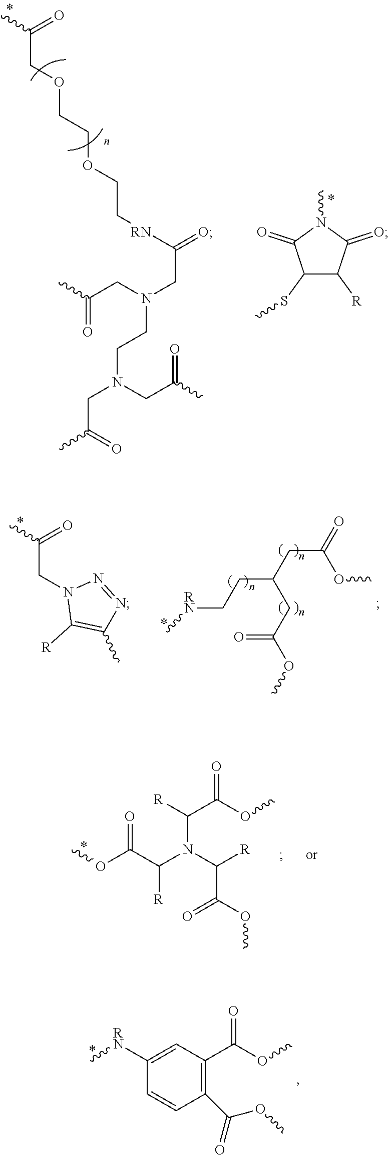

wherein R═H or C₁₋₆alkyl; and
n=0 to 12
or a stereoisomer thereof, wherein the (*) indicates the attachment site of R⁴⁰ to a subunit of the Amino Acid unit or a portion of the Linker Subunit L2 and the ( [symbol] ) indicates the attachment site of R⁴⁰ to the remainder of the PEG unit.

In some embodiments, provided is a Linker intermediate or Linker, wherein R⁴⁰ has one of the following structures:

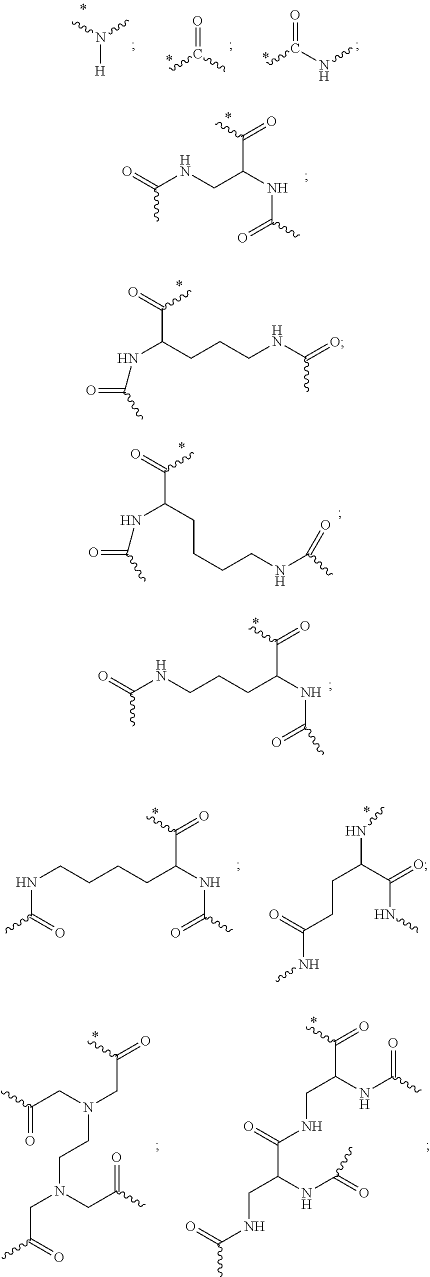

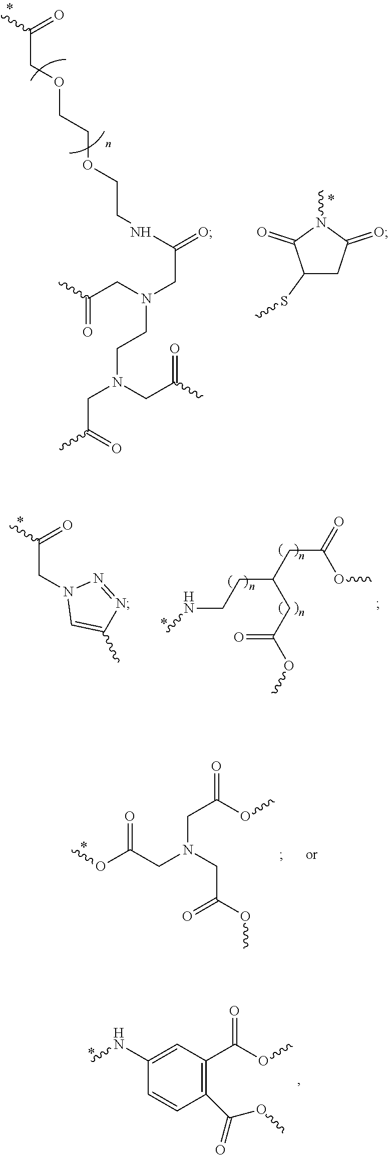

wherein n=0 to 12
or a stereoisomer thereof, wherein the (*) indicates the attachment site of R⁴⁰ to a subunit of the Amino Acid unit or a portion of the Linker Subunit L2 and the ( [symbol] ) indicates the attachment site of R⁴⁰ to the remainder of the PEG unit.

In some embodiments, provided is a Linker intermediate or Linker, wherein R⁴³—(NR⁴⁴R⁴⁵)ₙ₄₁, when R⁴³ is present, has one of the following structures:

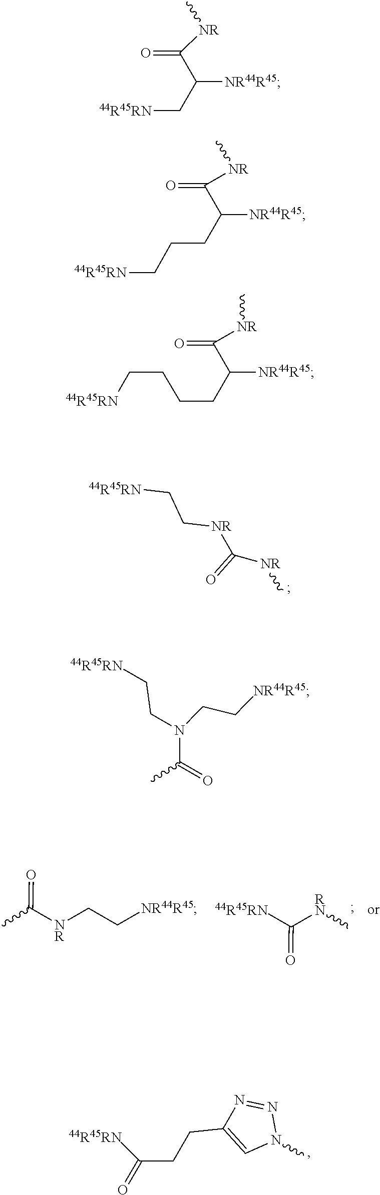

wherein R═H, C₁₋₆alkyl, polyhydroxyl, or substituted polyhydroxyl
or a stereoisomer thereof, wherein the ( [symbol] ) indicates the attachment site of R⁴³ to the remainder of the PEG unit.

In some embodiments, provided is a Linker intermediate or Linker, wherein R⁴³—(NR⁴⁴R⁴⁵)ₙ₄₁, when R⁴³ is present, has one of the following structures:

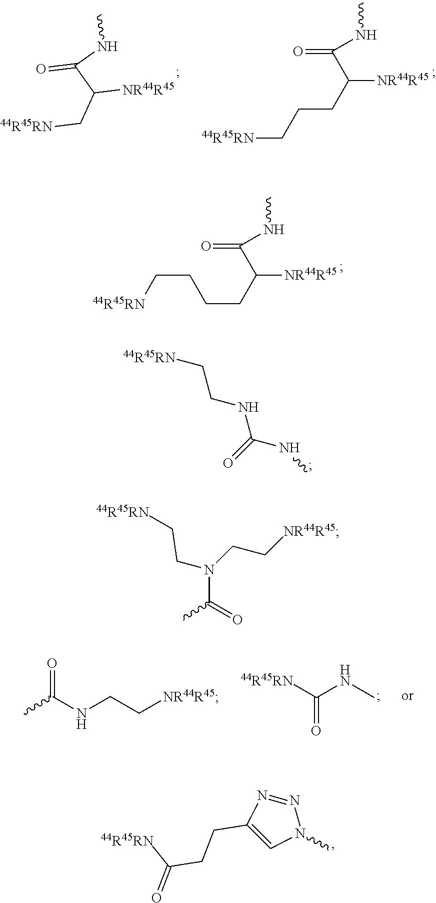

or a stereoisomer thereof, wherein the ( [symbol] ) indicates the attachment site of R⁴³ to the remainder of the PEG unit.

In some embodiments, provided is a Linker intermediate or Linker, wherein —NR⁴⁴R⁴⁵ has one of the following structures:

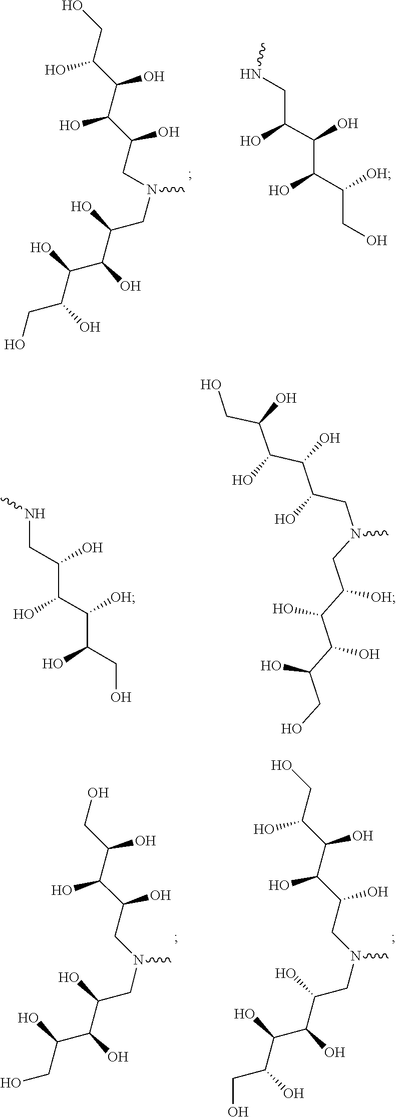

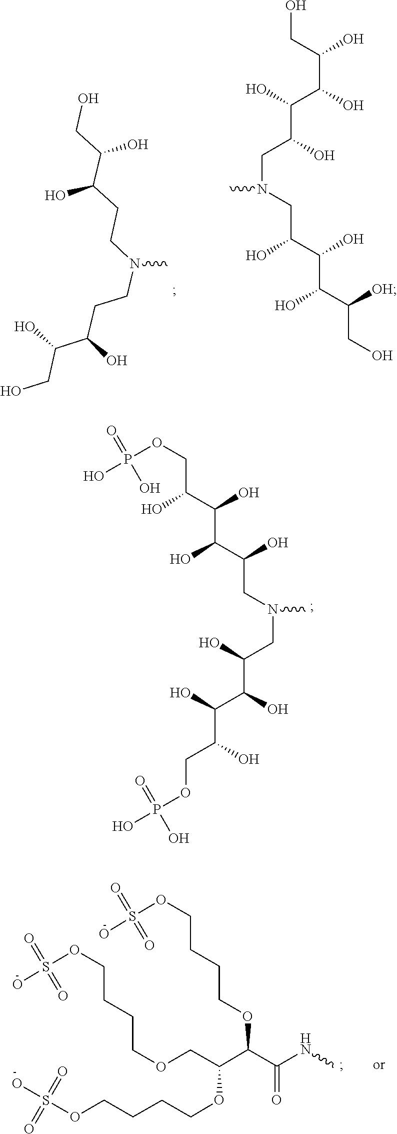

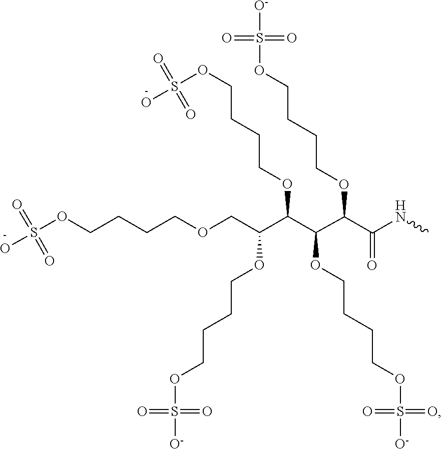

or a stereoisomer thereof, wherein the ( [symbol] ) indicates the attachment site of —NR⁴⁴R⁴⁵ to the remainder of the PEG unit.

In some embodiments, provided is a Linker intermediate or Linker, wherein the PEG unit has one of the following structures prior to attachment to the Amino Acid unit or to a portion of the Linker Subunit L2:

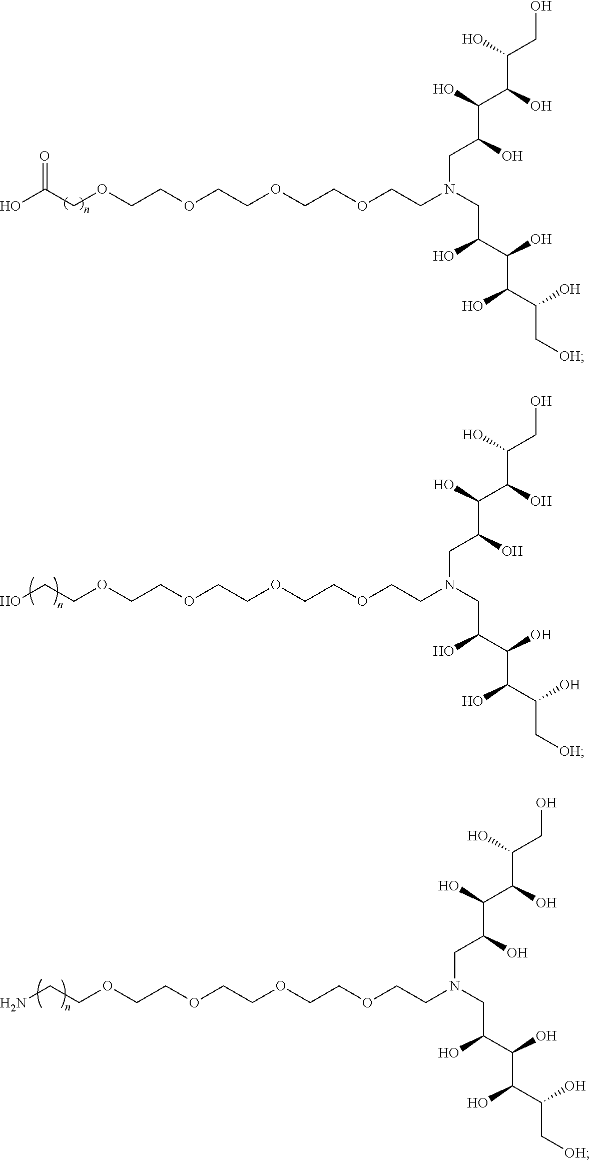

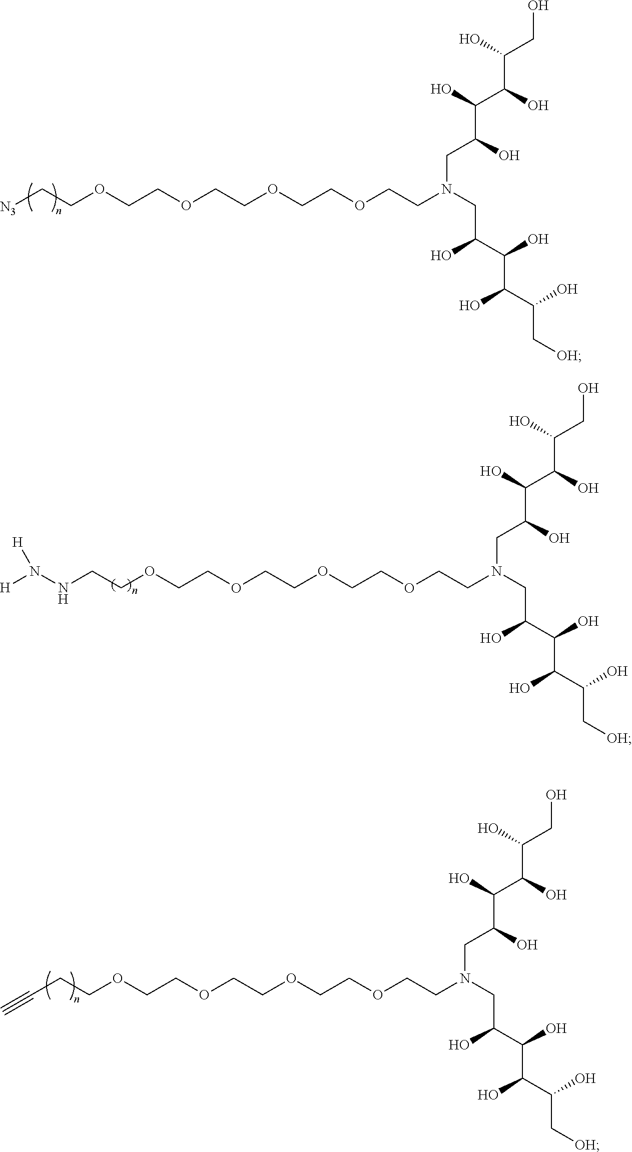

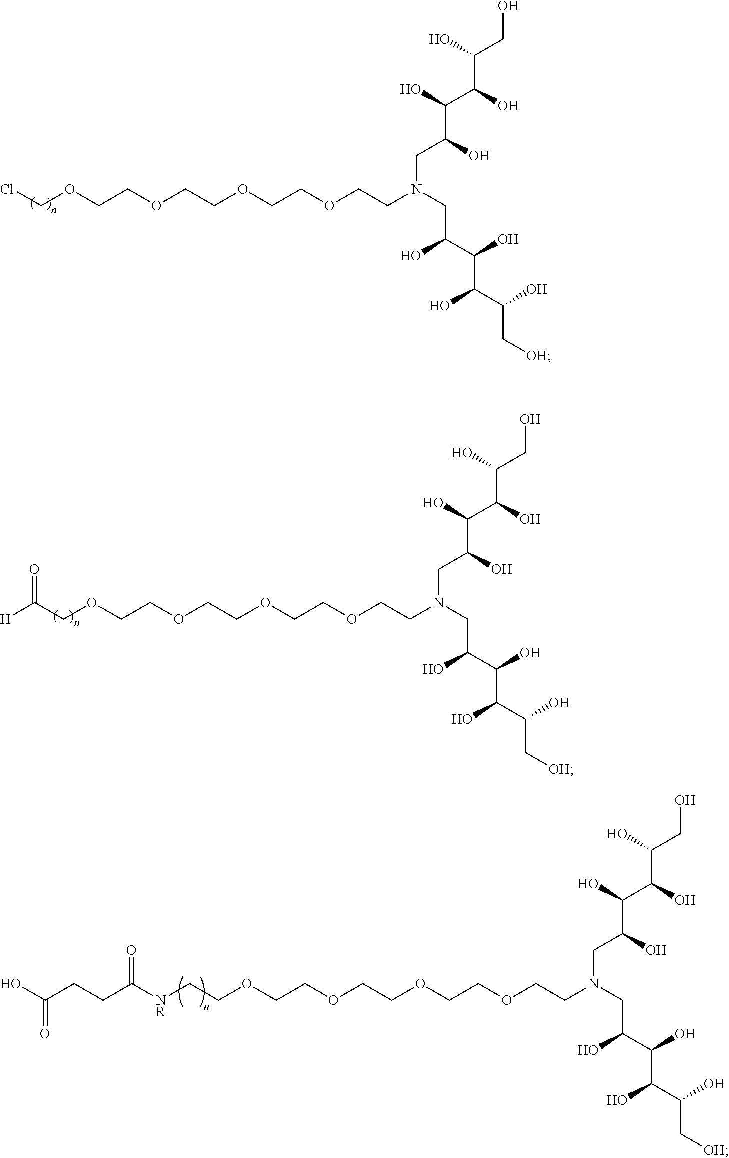

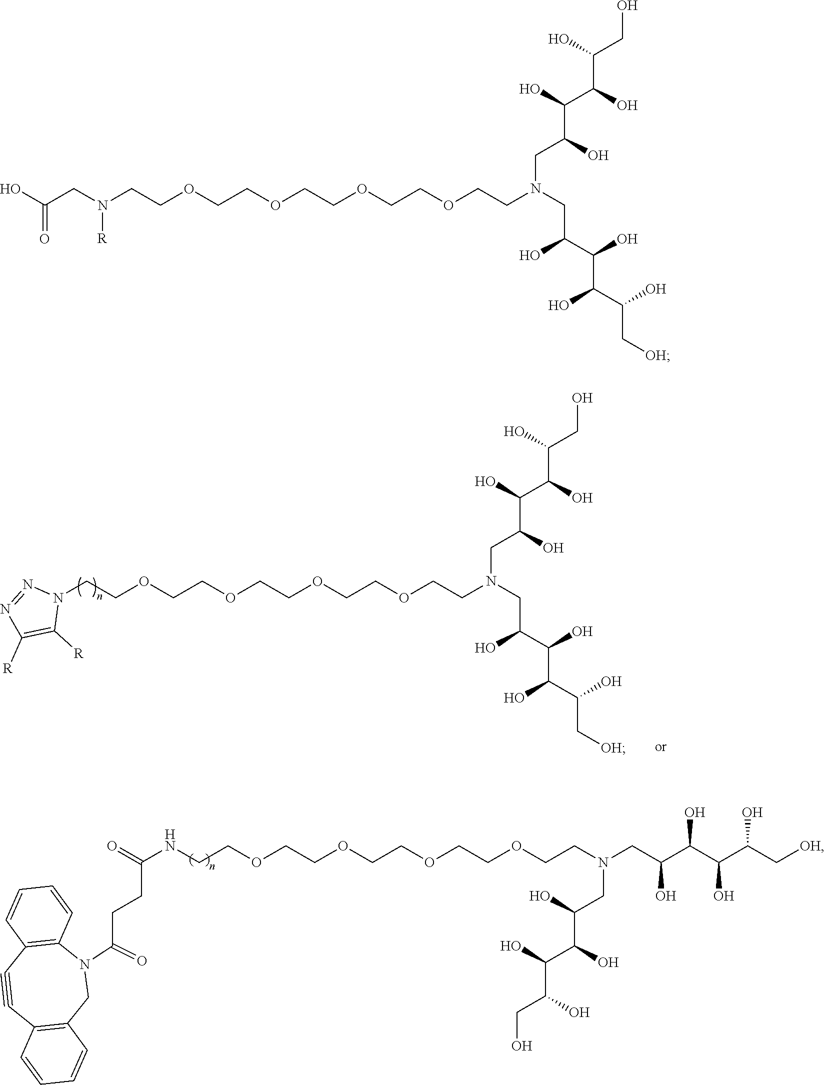

wherein R is H or alkyl, and n is 1 to 12.

In some embodiments, provided is a Linker intermediate or Linker, comprising a PEG unit having a formula selected from:

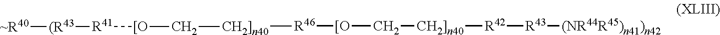

or a salt thereof, wherein:
R⁴⁰ is a functional group for attachment to a subunit of the Amino Acid unit or a portion of the Linker Subunit L2;
R⁴¹ and R⁴² are absent or are each, independently, C₁-C₆ alkylene;
each R⁴³ is, independently, absent or is selected from selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, a polyhydroxyl group, a substituted polyhydroxyl group, a —C(O)-polyhydroxyl group, or a substituted —C(O)— polyhydroxyl group, wherein optional substituents are selected from sulfate, phosphate, alkyl sulfate, and alkyl phosphate;
R⁴⁶ is selected from amino, amino-alkyl-amino, or —NH—C(O)—NH—S(O)₂—NH—;
the wavy line ( [symbol] ) indicates the attachment site to R⁴⁰
n40 is 1 to 26;
n41 is 1 to 6; and
n42 is 1 to 6.

In some embodiments, provided is a Linker intermediate or Linker, wherein the PEG unit has one of the following structures prior to attachment to the Amino Acid unit or to a portion of the Linker Subunit L2:

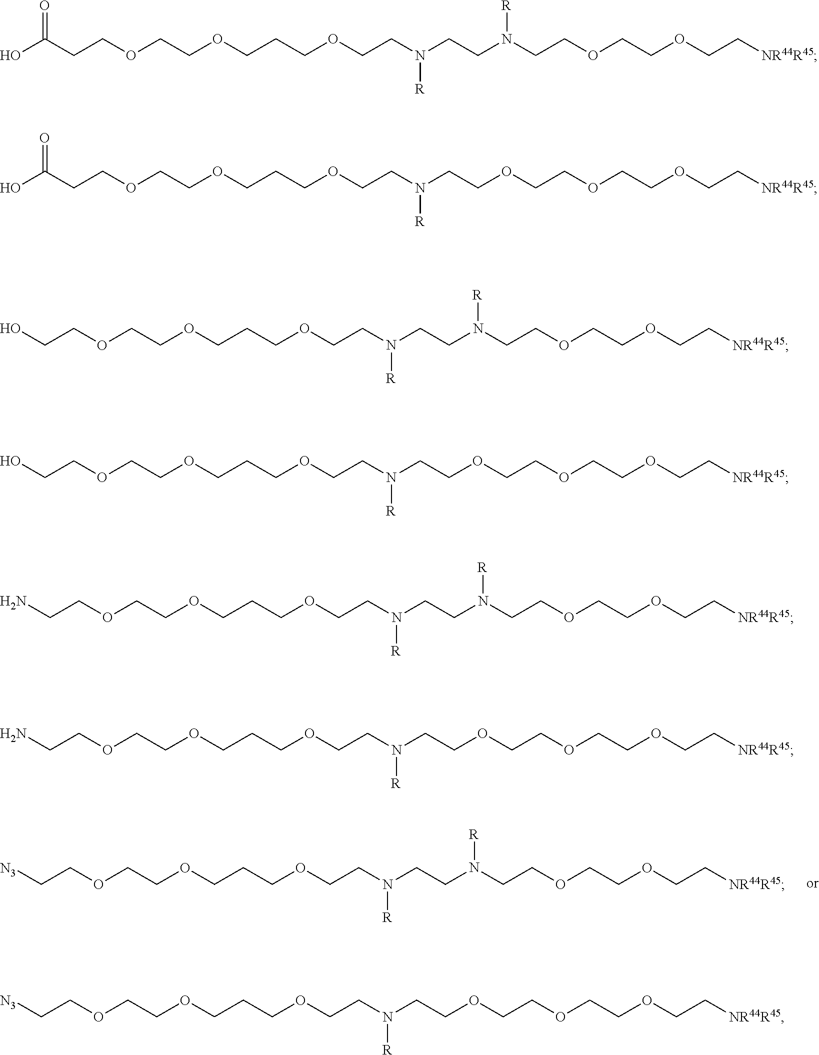

wherein R is H or alkyl, and n is 1 to 12.

In some embodiments, provided is a Linker intermediate or Linker, comprising a PEG unit having a formula selected from:

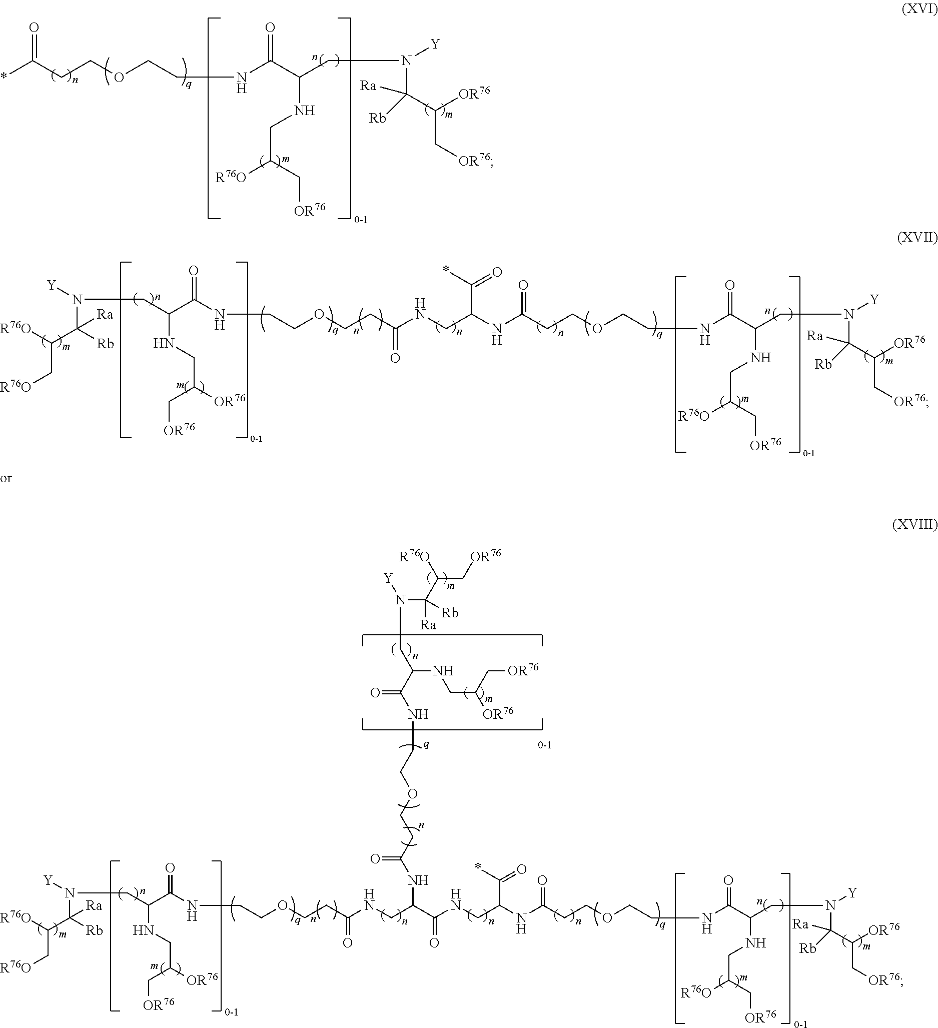

or a salt thereof, wherein:

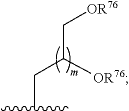

each Y is independently R⁷⁶ or
each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 1-26;
each m is independently 1 to 4;
each n is independently 1 to 4;
each v is independently 1 to 6; and
each * indicates an attachment site for a subunit of the Amino Acid unit (AA), the Linker subunit L2, or the Stretcher unit (L1).

In some embodiments, provided is a Linker intermediate or Linker, comprising a PEG unit having a formula selected from:

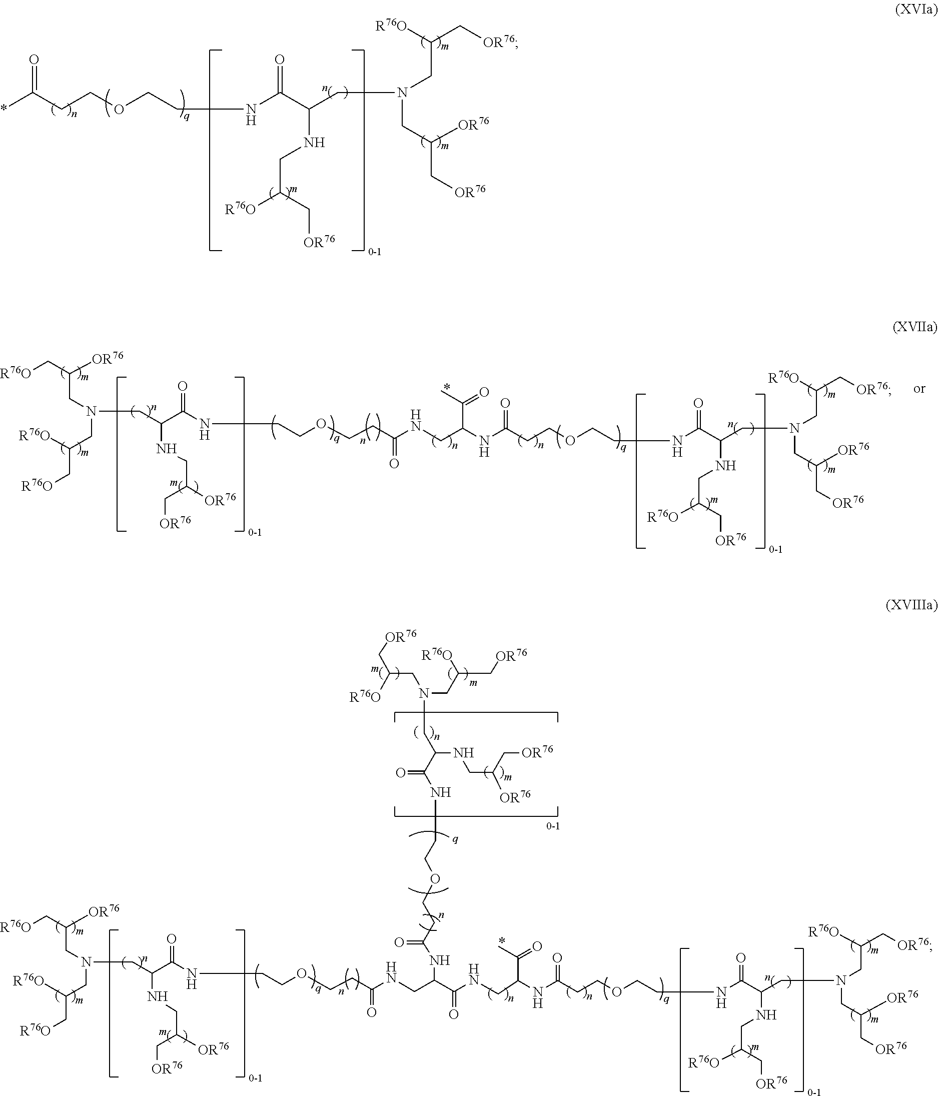

or a salt thereof, wherein:
   each R⁷⁶ is independently H, acetyl, —P(═O)(OH)₂, or —(CH₂),S(═O)₂(OH);
   each q is independently 1-26;
   each m is independently 1 to 4;
   each n is independently 1 to 4;
   each v is independently 1 to 6; and
   each * indicates an attachment site for a subunit of the Amino Acid unit (AA), the Linker subunit L2, or the Stretcher unit (L1).

In some embodiments, provided is a Linker intermediate or Linker, comprising a PEG unit having a formula selected from:

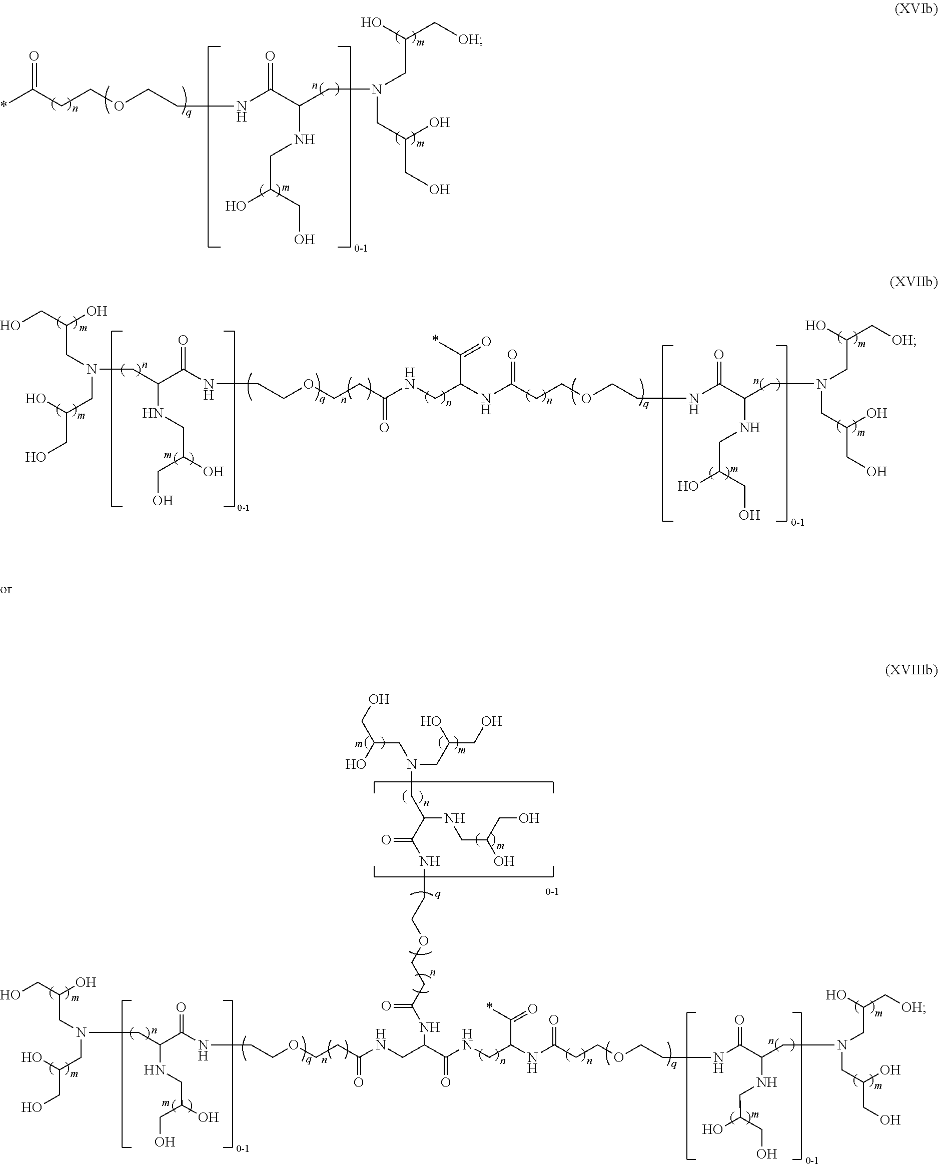

or a salt thereof, wherein:
   each q is independently 1-26;
   each m is independently 1 to 4;
   each n is independently 1 to 4; and
   each * indicates an attachment site for a subunit of the Amino Acid unit (AA), the Linker subunit L2, or the Stretcher unit (L1).

In some embodiments, provided is a Linker intermediate or Linker, wherein Y is R⁷⁶.

In some embodiments, provided is a Linker intermediate or Linker, wherein Y is

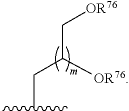

In some embodiments, provided is a Linker intermediate or Linker, wherein each Rₐ and R_{b} is independently H.

In some embodiments, provided is a Linker intermediate or Linker, wherein Rₐ and R_{b}are taken together with the carbon to which they are attached to form an oxo group.

In some embodiments, provided is a Linker intermediate or Linker, wherein q is 10-20.

In some embodiments, provided is a Linker intermediate or Linker, wherein q is 12.

In some embodiments, provided is a Linker intermediate or Linker, wherein the PEG unit is selected from the following, or a salt thereof:

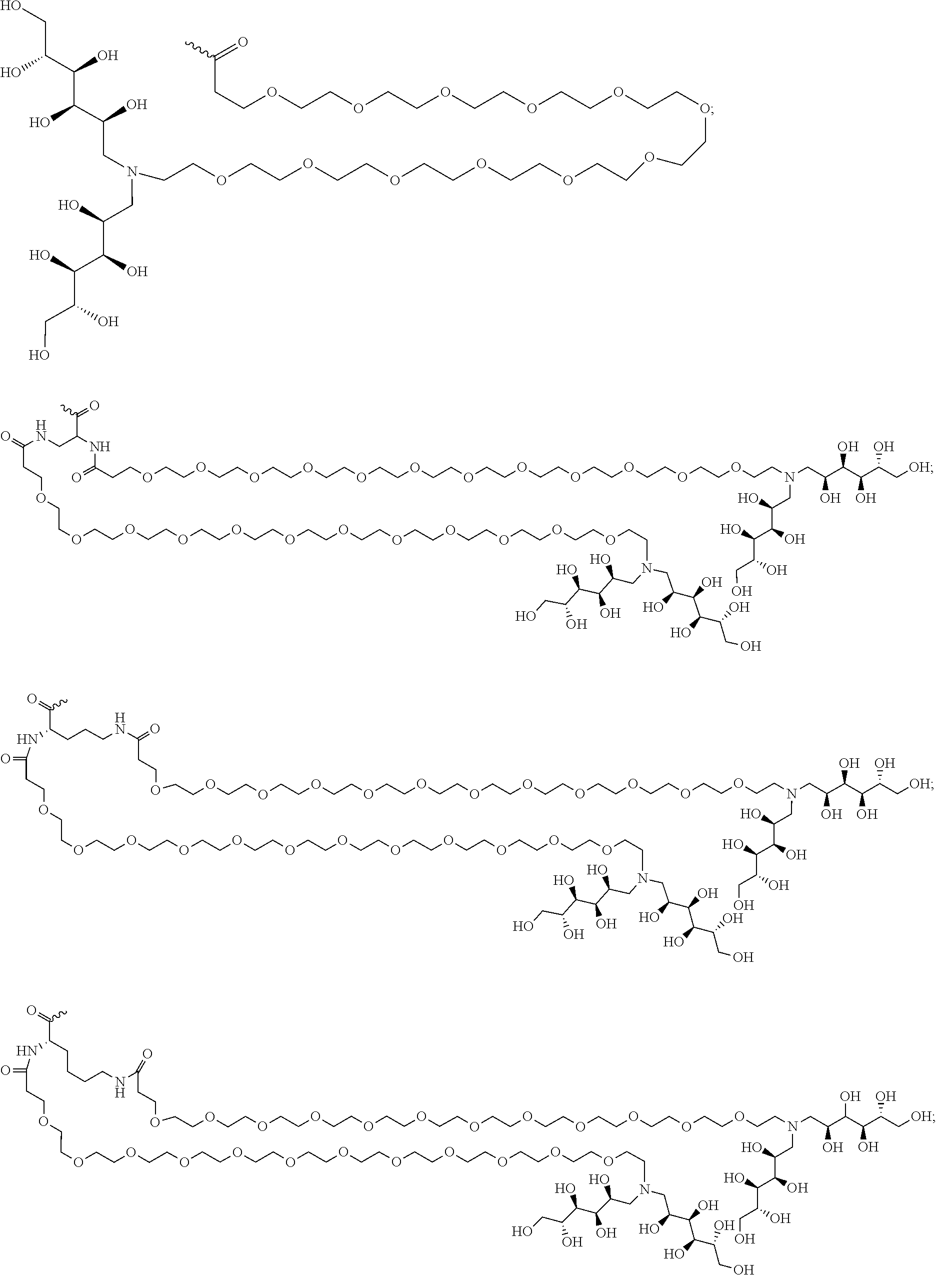

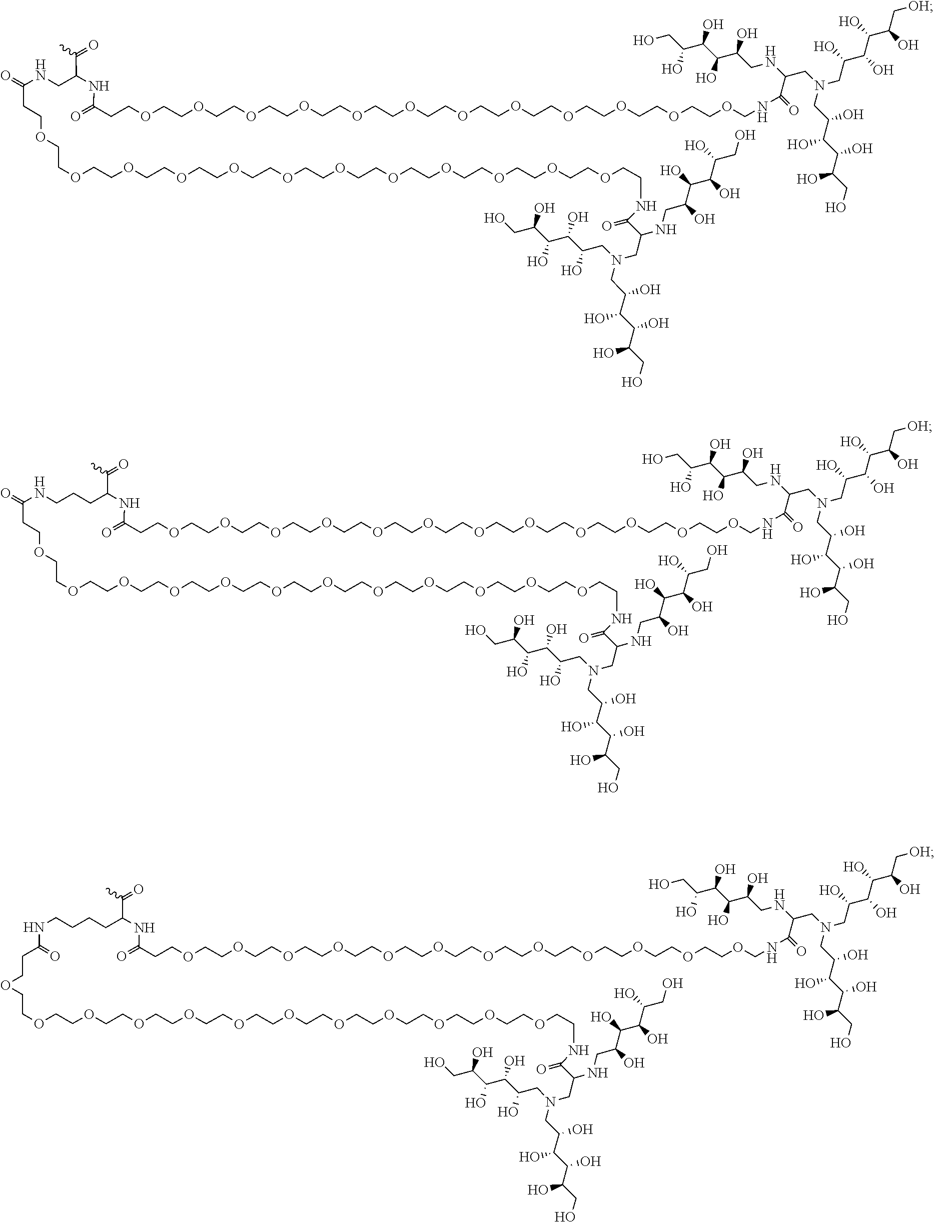

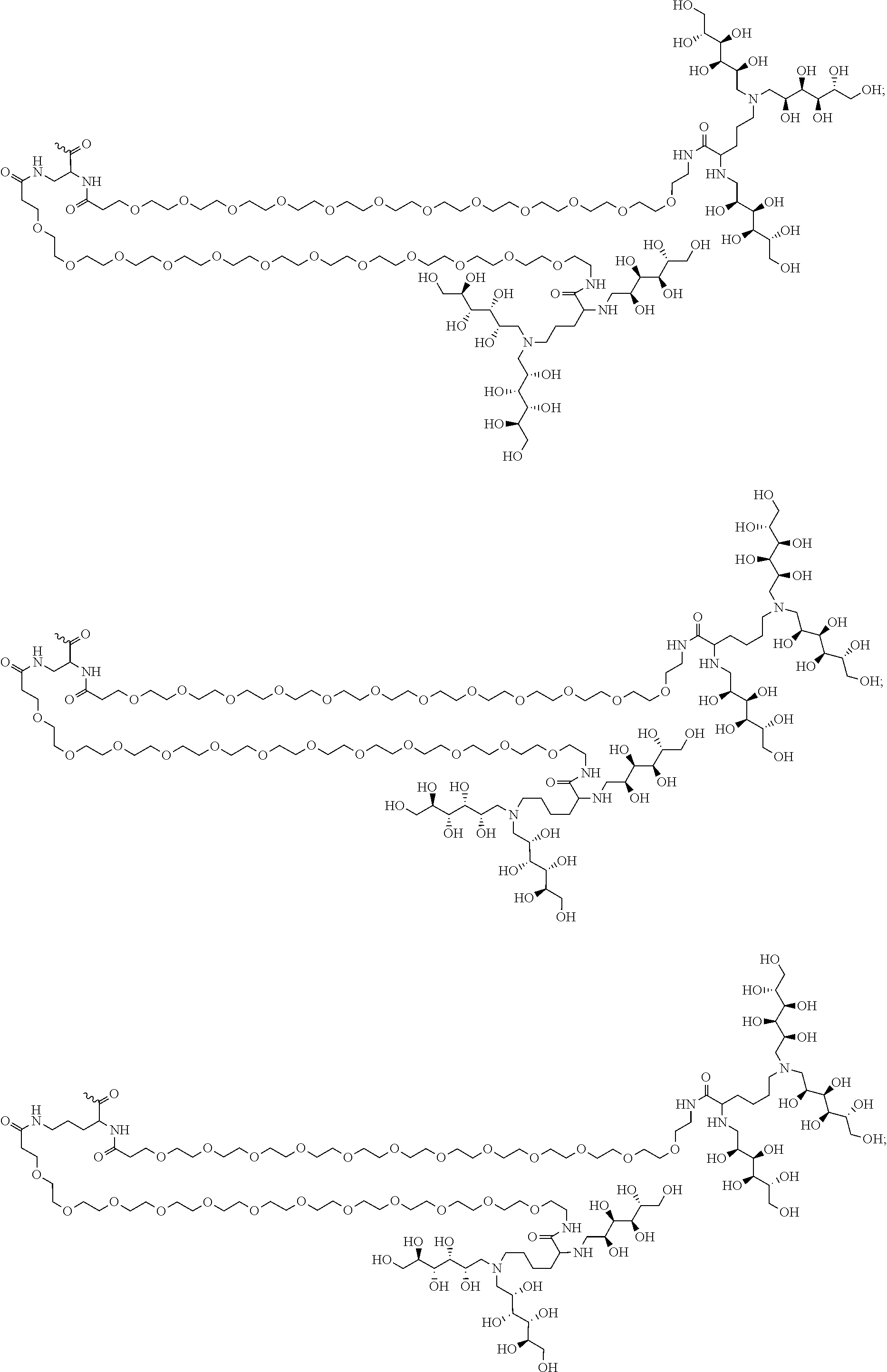

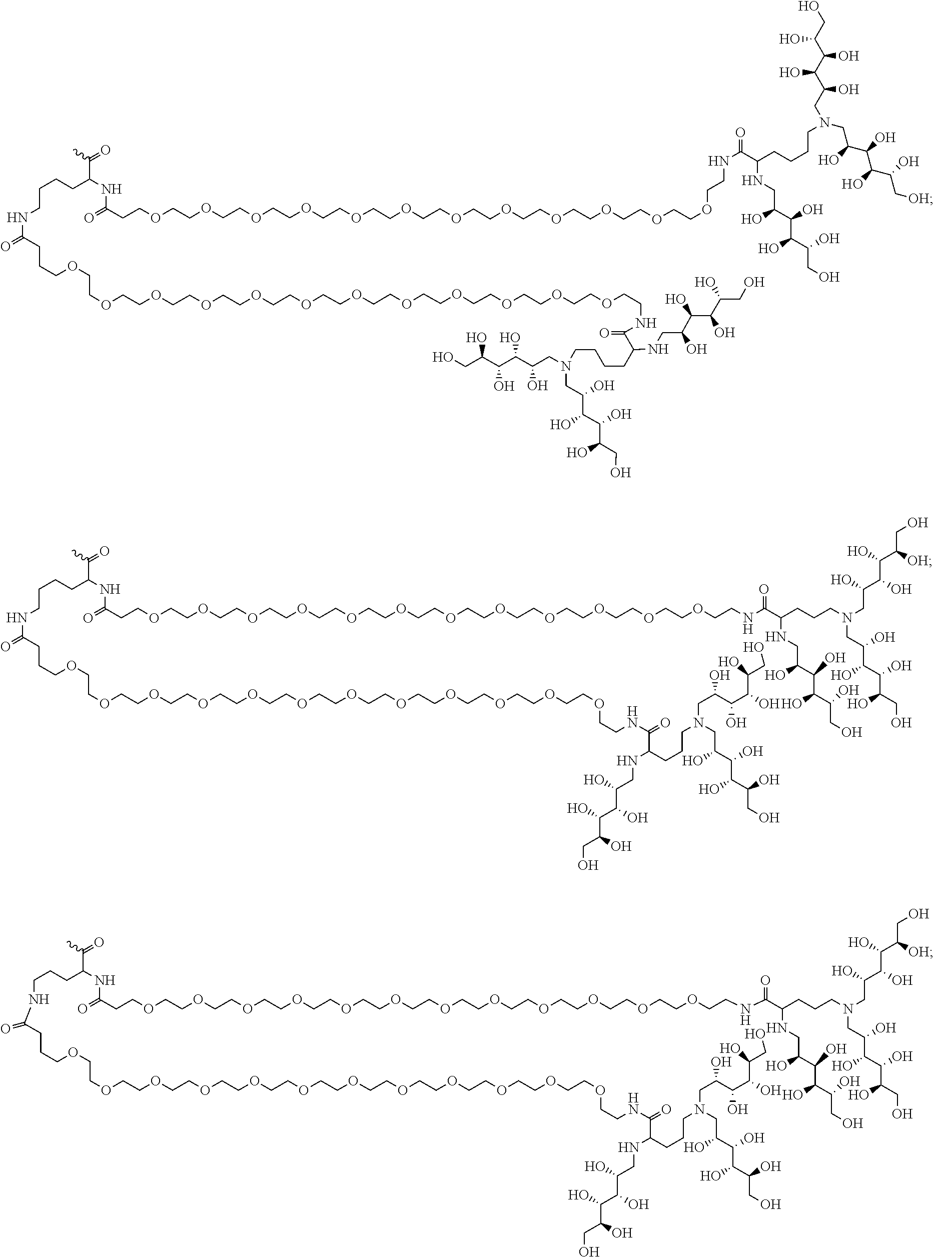

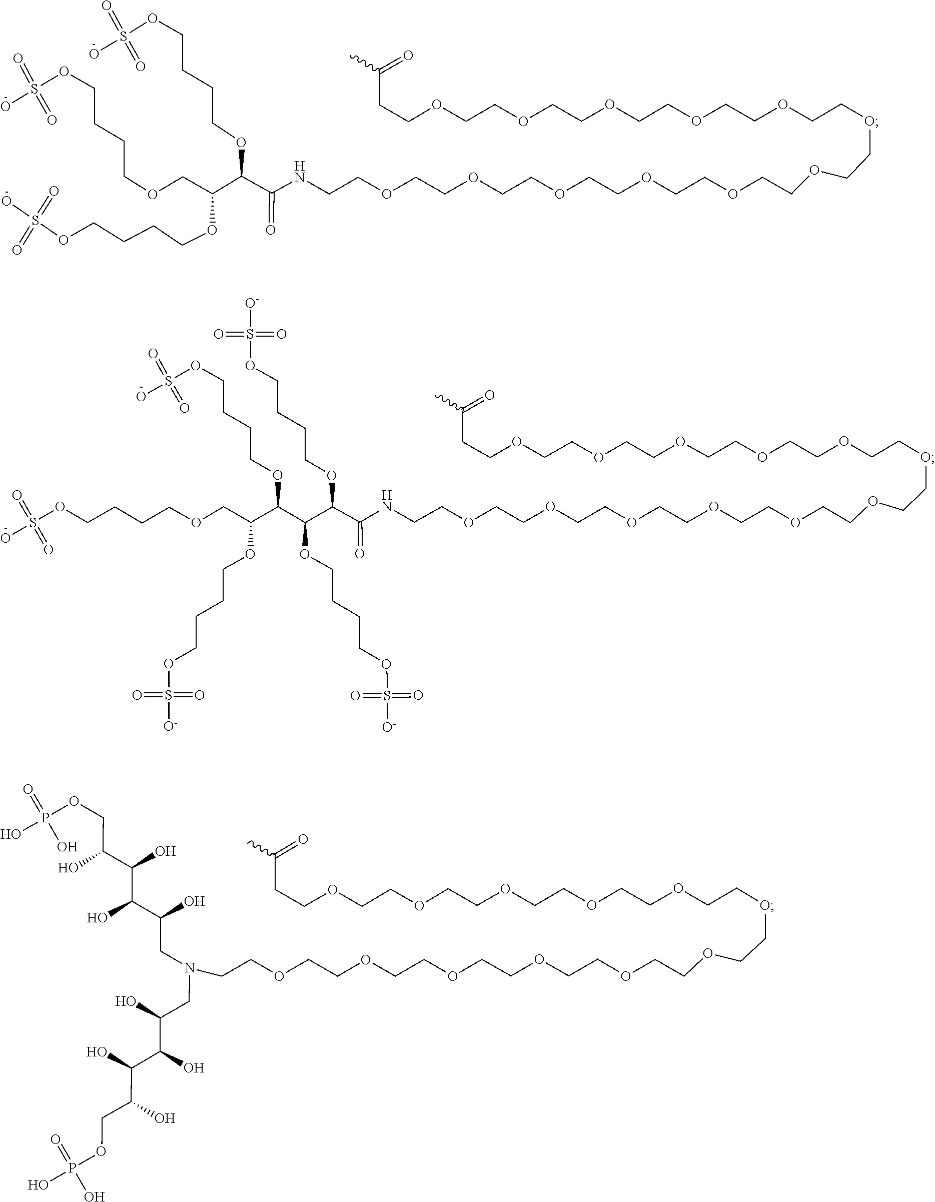

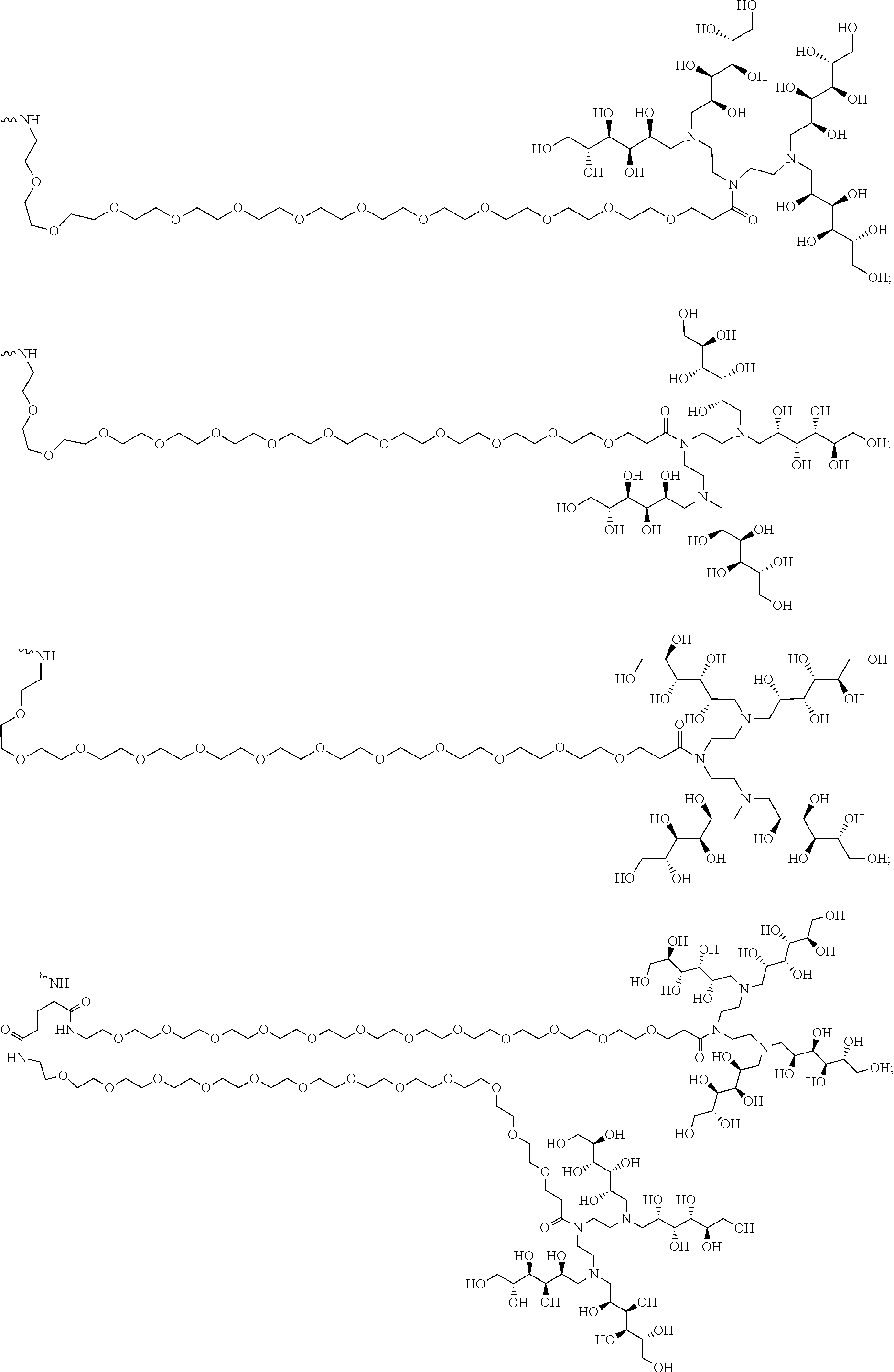

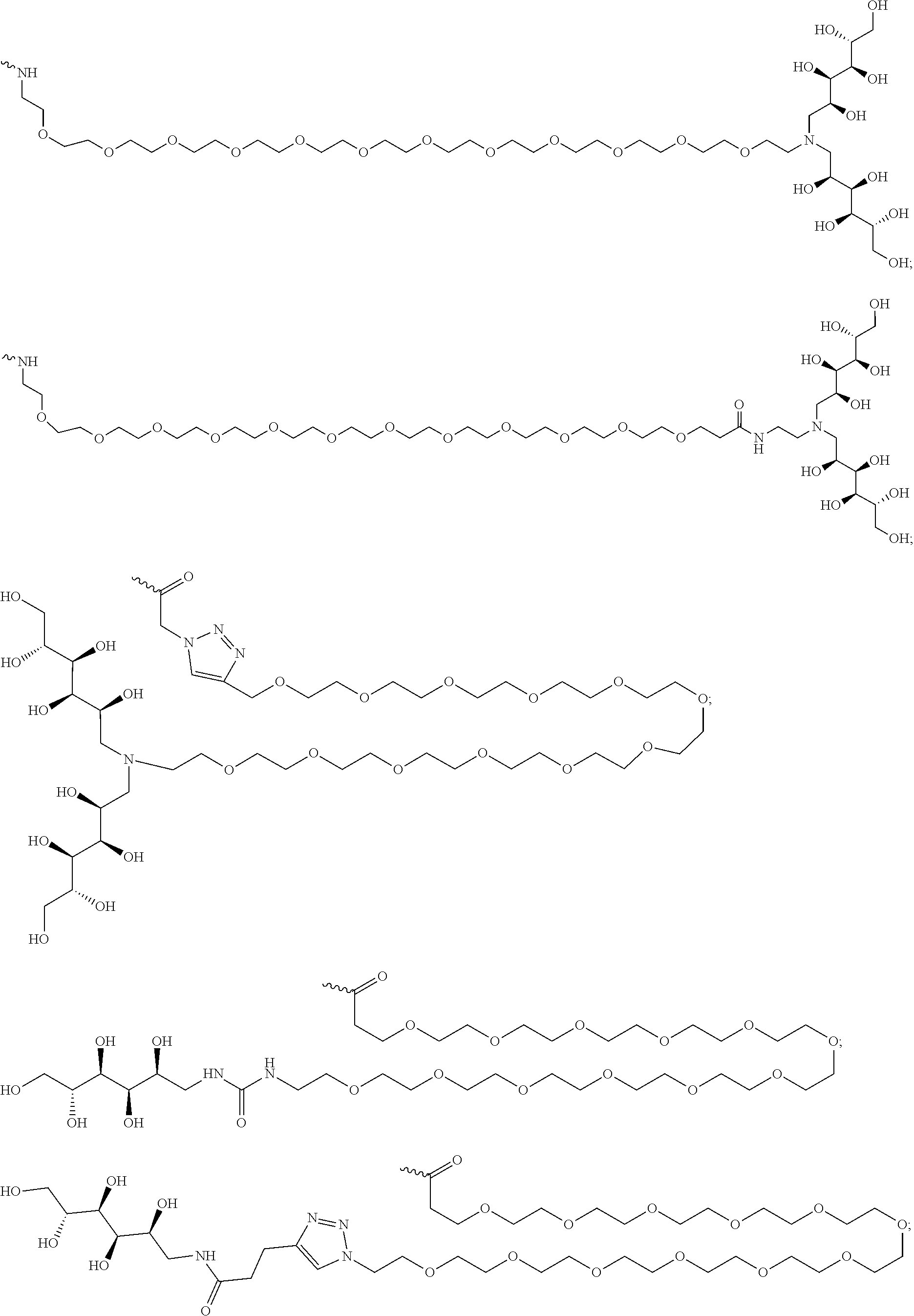

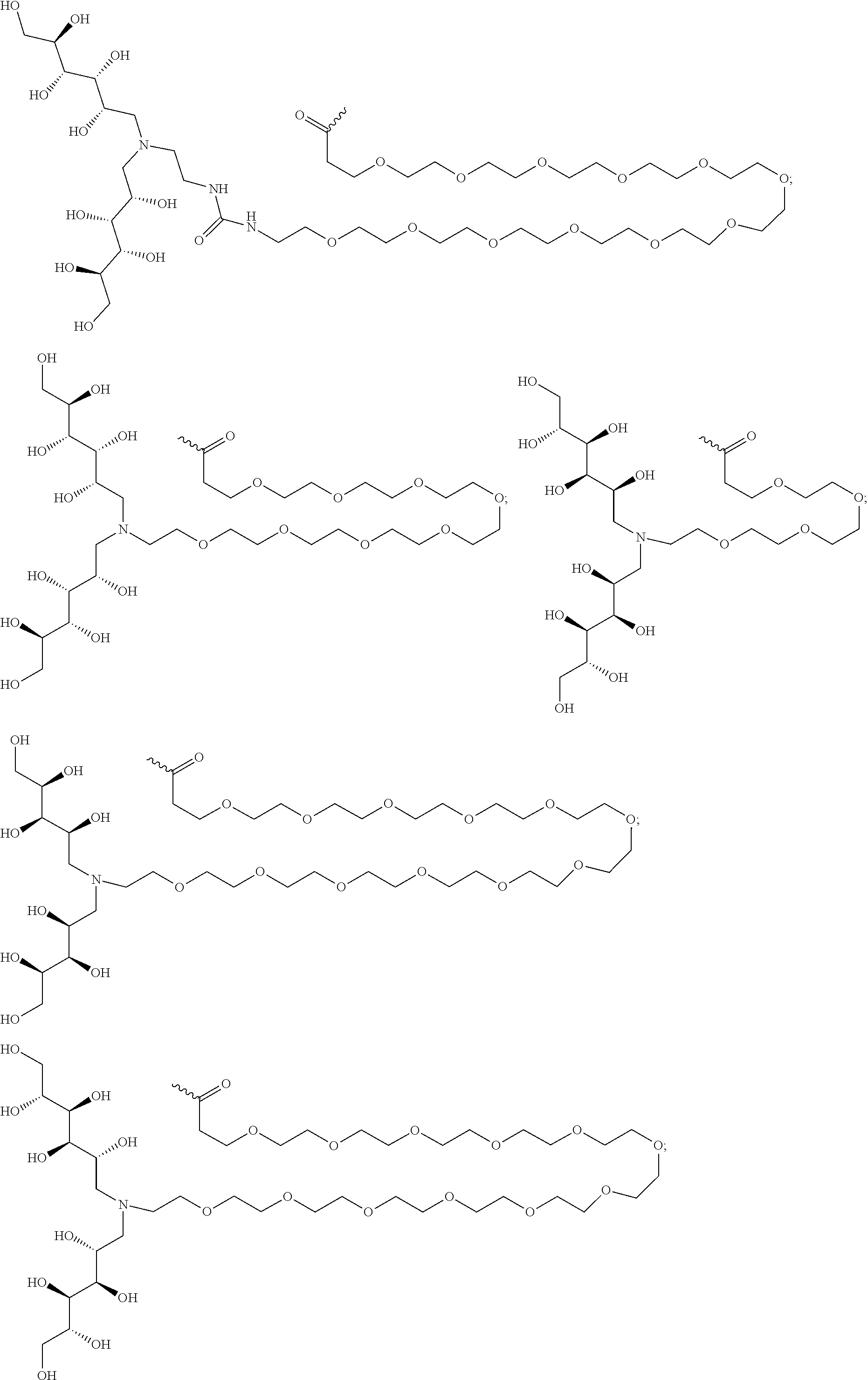

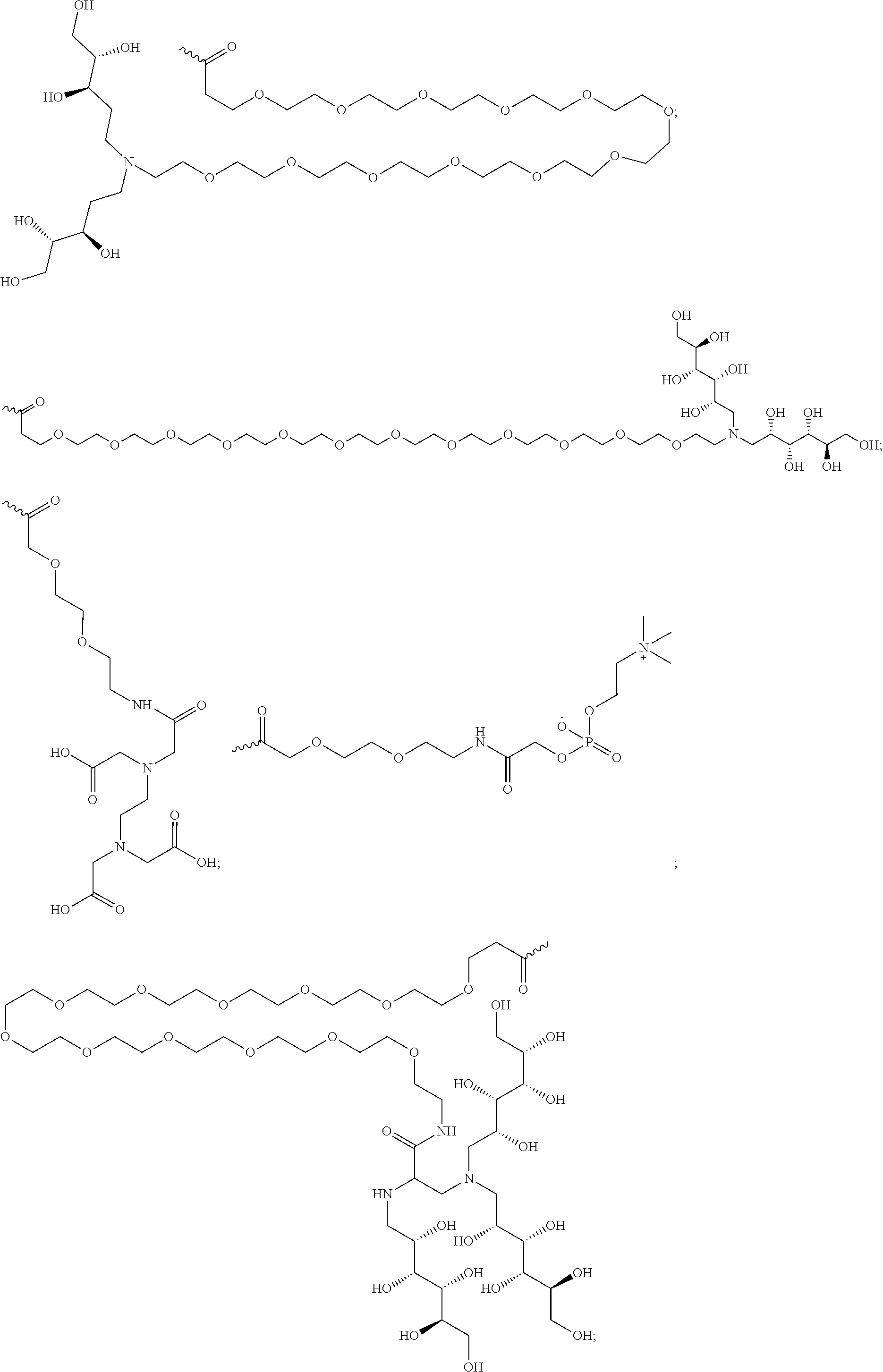

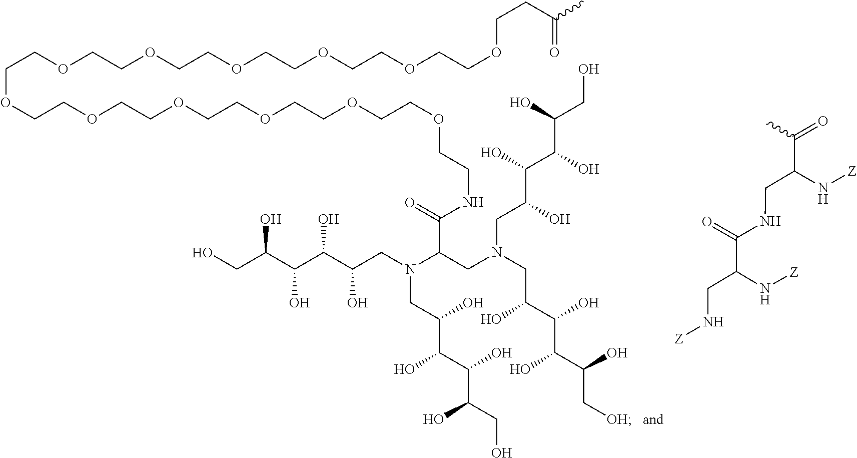

wherein each Z is attached at * and is individually selected from:

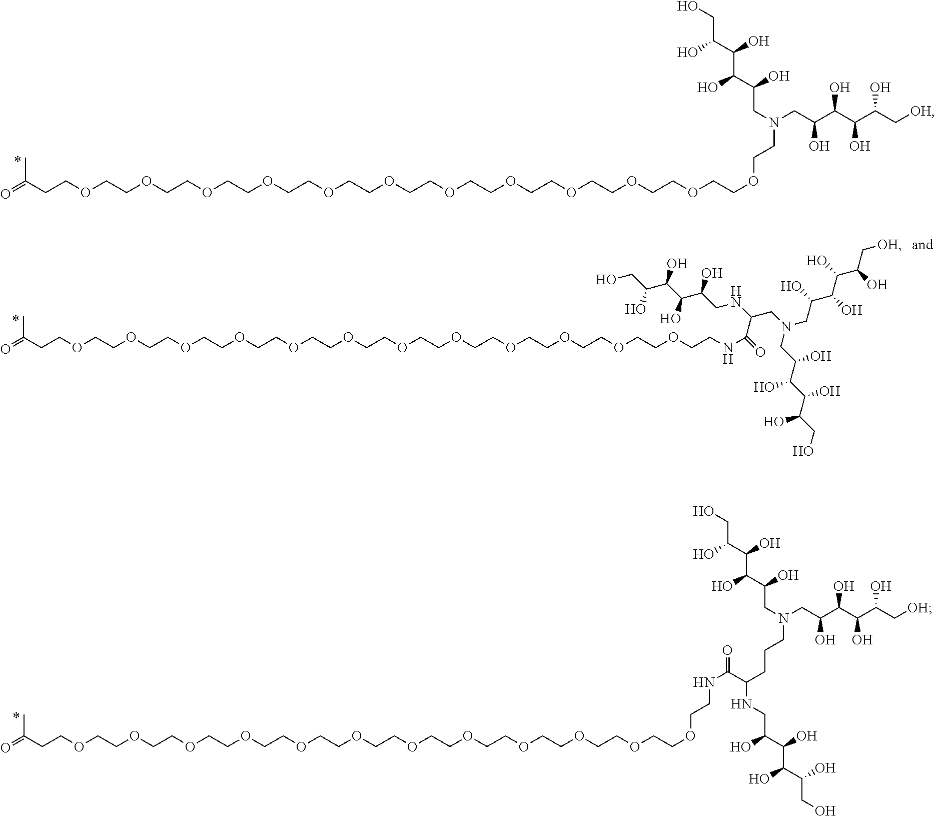

wherein each [symbol] indicates an attachment site to a subunit of the Amino Acid unit (AA), a [symbol] portion of the Linker subunit L2, or the Stretcher unit (L1).

### Carboxyl Units

In some embodiments, a Linker comprises a Carboxyl unit. A Carboxyl unit can be a subunit of an Amino Acid unit or attached to a portion of a Linker Subunit L2. In some embodiments, a Carboxyl unit has the following general formula (XXXX):

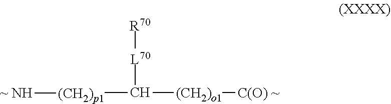

or a salt thereof, wherein L⁷⁰ is selected from C₁-C₈ alkylene, C₁-C₈ alkylene-C(O)—, —C(O)—C₁-C₈ alkylene-, and —C(O)—C₁-C₈ alkylene-C(O)—; R⁷⁰ is —NR⁷¹(R⁷²R⁷³), wherein R⁷¹ is selected from H, C₁-C₁₂ alkyl, substituted C₁-C₁₂ alkyl, or polyethylene glycol (optionally having 1 to 12 ethylene glycol subunits); R⁷² is absent or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted arylene or optionally substituted heteroarylene; R⁷³ is a carboxyl or polycarboxyl; and each of p1 and o1 are independently selected from 0 to 2. As used herein, the term “polycarboxyl” refers to a group that contains from 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide. As used herein, polycarboxyl includes the carboxylate forms.

In some embodiments, R⁷⁰ is ˜NR⁷¹(R⁷⁵—(R⁷³)₂), wherein R⁷¹ is selected from H, C₁-C₁₂ alkyl, substituted C₁-C₁₂ alkyl, or polyethylene glycol (optionally having 1 to 12 ethylene glycol subunits); R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted arylene or optionally substituted heteroarylene; each R⁷³ is a carboxyl or polycarboxyl; and each of p1 and 01 are independently selected from 0 to 2.

In some embodiments, R⁷⁰ is ˜N(R⁷⁴-R⁷³)(R⁷²-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted arylene or optionally substituted heteroarylene; each R⁷³ is independently carboxyl or polycarboxyl; and each of p1 and 01 are independently selected from 0 to 2.

In some of the above embodiments, R⁷³ can be selected from:

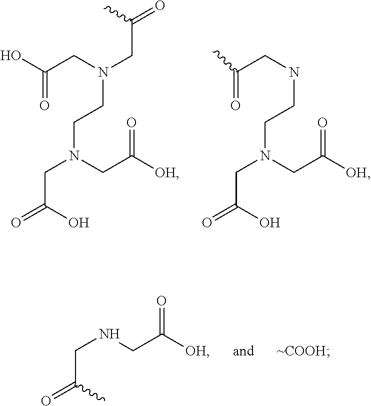

wherein the wavy line indicates a bond to R⁷², R⁷⁴ or R⁷⁵.

### Linker Subunit L2

The Linkers comprise at least one Linker Subunit L2, each Linker Subunit L2 having an attachment site for at least one Drug unit (D), as further described herein. In some embodiments, a Drug unit (D) is attached to each attachment site for a Drug unit on a Linker Subunit L2. In various embodiments, Linker Subunit L2 may be a cleavable linker subunit or a non-cleavable linker subunit. A Linker Subunit L2 also has an attachment site for an Amino Acid unit (AA) or a Stretcher unit (L1).

In some embodiments, a Linker Subunit L2 includes a Polar unit, such as a Sugar unit, a PEG unit or a Carboxyl unit. In some embodiments, a Linker Subunit L2 does not include a Polar unit, wherein an Amino Acid unit includes a Polar unit. In some embodiments, both a Linker Subunit L2 and an Amino Acid unit (if present) include a Polar unit.

In some embodiments, the Linker Subunit L2 is a cleavable linker subunit. As used herein, the term “cleavable” refers to a metabolic process or reaction inside a cell or in the extracellular milieu, whereby the covalent attachment between a Drug unit (e.g., a cytotoxic agent) and the Linker Subunit L2 or portion thereof is broken, resulting in the free Drug unit, or other metabolite of the Linker Subunit L2-Drug unit dissociated from the remainder of the Linker Subunit L2.

In some embodiments, the Linker Subunit L2 includes a protease cleavable linker subunit, an acid-cleavable linker subunit, a disulfide linker subunit, a disulfide-containing linker subunit, or a disulfide-containing linker subunit having a dimethyl group adjacent the disulfide bond (e.g., an SPDB linker) (see, e.g., Jain et al., Pharm. Res. 32:3526-3540 (2015); Chari et al., Cancer Res. 52:127-131 (1992); U.S. Pat. No. 5,208,020), a cleavable self-stabilizing linker (see, e.g., WO2018/031690 and WO2015/095755 and Jain et al., Pharm. Res. 32:3526-3540 (2015)), and/or a cleavable hydrophilic linker (see, e.g., WO2015/123679). In some embodiments, the Linker Subunit L2 includes a photolabile linker subunit. In some embodiments, the Linker Subunit L2 has a non-cleavable linker unit (see, e.g., WO2007/008603).

In some embodiments, the Linker Subunit L2 is a cleavable linker that is cleavable under intracellular conditions, such that cleavage of or within the Linker Subunit L2 releases the Drug unit from Linker Subunit L2 or the remainder of Linker Subunit L2 in the intracellular environment. For example, in some embodiments, Linker Subunit L2 is cleavable by a cleaving agent that is present in the intracellular environment (e.g., within a lysosome or endosome or caveolae). As used herein, the terms “cleavable under intracellular conditions”, “intracellularly cleaved” and “intracellular cleavage” refer to a metabolic process or reaction inside a cell, whereby the covalent attachment between a Drug unit (e.g., a cytotoxic agent) and the Linker Subunit L2 or portion thereof is broken, resulting in the free Drug unit, or other metabolite of the Linker Subunit L2-Drug unit dissociated from the remainder of the Linker Subunit L2 inside the cell. The cleaved moieties of the conjugate are thus intracellular metabolites.

In some embodiments, a linkage between the Linker Subunit L2 and the Drug unit can be enzymatically cleaved by one or more enzymes, including a tumor-associated protease, to liberate the Drug unit (D). Linker Subunit L2 can be, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). Intracellular cleaving agents can include cathepsins B, C and D and plasmin, all of which are known to hydrolyze dipeptide drug derivatives resulting in the release of active drug inside target cells (see, e.g., Dubowchik and Walker, 1999, Pharm. Therapeutics 83:67-123). Peptidyl linkers can be cleavable by enzymes that are present in target antigen-expressing cells. For example, a peptidyl linker subunit that is cleavable by the thiol-dependent protease cathepsin-B, which is highly expressed in cancerous tissue, can be used (e.g., having a Phe-Leu, Val-Ala, Val-Cit or Gly-Phe-Leu-Gly peptide (SEQ ID NO: 47)).

Typically, a peptidyl linker is at least one amino acid long or at least two amino acids long. In certain embodiments, the peptidyl linker is a dipeptide, tripeptide, tetrapeptide or pentapeptide. In certain embodiments, a peptidyl linker subunit can comprise only natural amino acids. In some embodiments, For example, a peptidyl linker subunit can have a Phe-Leu, Val-Ala, Val-Cit or Gly-Phe-Leu-Gly peptide (SEQ ID NO: 47). Other such cleavable linkers are described, for example, in U.S. Pat. No. 6,214,345. In specific embodiments, the peptidyl linker that is cleavable by an intracellular protease comprises a Val-Cit peptide or a Phe-Lys peptide (see, e.g., U.S. Pat. No. 6,214,345) or Gly-Gly-Phe-Gly linker (SEQ ID NO: 43) (see, e.g., US Published Application No. 2015/0297748). One advantage of using intracellular proteolytic release of the Drug unit is that the activity of the Drug unit is typically attenuated when conjugated and the serum stabilities of the conjugates are typically high. See also U.S. Pat. No. 9,345,785.

In some embodiments, a peptidyl linker subunit can comprise only non-natural amino acids. In some embodiments, a peptidyl linker subunit can comprise a natural amino acid linked to a non-natural amino acid. In some embodiments, a peptidyl linker subunit can comprise a natural amino acid linked to a D-isomer of a natural amino acid. In some embodiments, at least one amino acid of a peptidyl linker subunit is an L-amino acid. In some embodiments, at least amino acid is a D-amino acid.

In some embodiments, a peptidyl linker subunit contains one or more the following: glycine and/or L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and a Polar unit (including a PEG unit(s) attached to glycine or an L-amino acid(s)). In some embodiments, a peptidyl linker subunit contains one or more the following: glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and a Polar unit (including a PEG unit(s) attached to glycine or a D-amino acid(s)). In some embodiments, a peptidyl linker subunit contains one or more the following: glycine and/or a mixture of L-amino acids and D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and a Polar unit (including a PEG unit(s) attached to glycine or an amino acid(s)).

In some embodiments, a peptidyl linker subunit contains one or more the following: glycine and/or natural L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar unit, such as a Sugar unit, or a Carboxyl unit or a PEG unit attached to glycine or an L-amino acid. In some embodiments, a peptidyl linker subunit contains one or more the following: glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar unit, such as a Sugar unit, or a Carboxyl unit or a PEG unit attached to glycine or an D-amino acid.

In some embodiments, an amino acid of a peptidyl linker subunit has the formula denoted below in the square brackets:

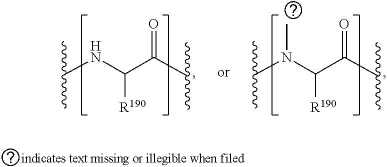

wherein R₁₉₀ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

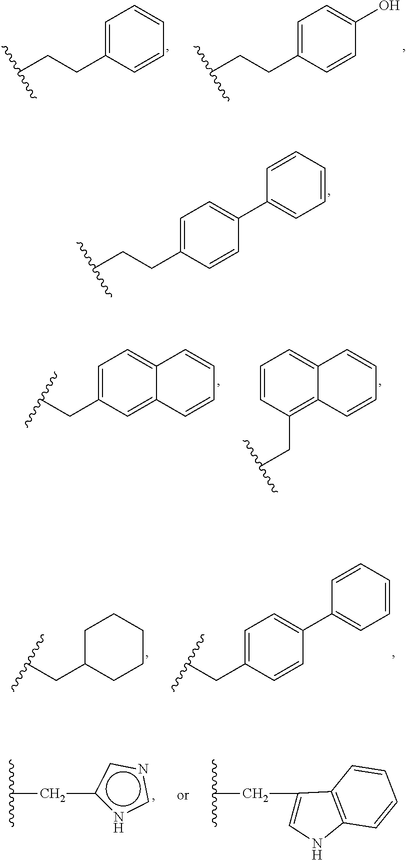

In some embodiments, a peptidyl linker subunit includes one or more of the following L-(natural) amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar unit, such as a Sugar unit, or a Carboxyl unit or a PEG unit attached to glycine or a natural amino acid.

In some embodiments, a peptidyl linker subunit does not contain cysteine. In some embodiments, a peptidyl linker does not contain proline.

In some embodiments, a peptidyl linker subunit includes one or more of the following D-isomers of these natural amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar unit, such as a Sugar unit, or Carboxyl unit or a PEG unit attached to glycine or a D-amino acid.

In some embodiments, a peptidyl linker subunit includes one or more of the following amino acids: alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, p-alanine, aminoalkanoic acid, aminoalkynoic acid, amino alkanedioic acid, aminobenzoic acid, amino-heterocyclo-alkanoic acid, heterocyclo-carboxylic acid, citrulline, statine, diaminoalkanoic acid, and derivatives thereof; and at least one Polar unit, such as a Sugar unit, or a Carboxyl unit or a PEG unit attached to an amino acid(s). Illustrative of examples of derivatives of such amino acids are set forth below in the section describing the Amino Acid subunit.

In some embodiments, a peptidyl linker subunit contains a Sugar unit as part of a peptide that is cleavable. For example, a Sugar unit containing lysine or citrulline as a part of a cleavable peptide. In some embodiments, a peptidyl linker subunit contains a Carboxyl unit as part of a peptide that is cleavable. For example, a Carboxyl unit containing lysine or citrulline as a part of a cleavable peptide.

In some embodiments, a cleavable linker subunit is pH-sensitive, i.e., sensitive to hydrolysis at certain pH values. Typically, a pH-sensitive linker subunit is hydrolyzable under acidic conditions. For example, an acid-labile linker subunit that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linker subunits are relatively stable under neutral pH conditions, such as those in the blood, but are unstable at below pH 5.5 or 5.0, the approximate pH of the lysosome. In certain embodiments, a hydrolyzable linker unit is a thioether linker (such as, for example, a thioether attached to the Drug unit via an acylhydrazone bond (see, e.g., U.S. Pat. No. 5,622,929)).

In some embodiments, a Linker Subunit L2 is cleavable under reducing conditions (e.g., a disulfide linker subunit). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, a Linker Subunit L2 is a malonate linker (Johnson et al., 1995, Anticancer Res. 15:1387-93), a maleimidobenzoyl linker (Lau et al., 1995, Bioorg-Med-Chem. 3(10):1299-1304), or a 3′—N-amide analog (Lau et al., 1995, Bioorg-Med-Chem. 3(10):1305-12). In some embodiments, the Linker Subunit L2 is not cleavable, such as a maleimidocaproyl linker, and the Drug unit is released by metabolic degradation of the Drug-Linker. (See, e.g., U.S. Publication No. 2005/0238649.)

In some embodiments, a Linker Subunit L2 is not substantially sensitive to the extracellular environment. As used herein, “not substantially sensitive to the extracellular environment,” in the context of a Linker Subunit L2, means that no more than about 20%, typically no more than about 15%, more typically no more than about 10%, and even more typically no more than about 5%, no more than about 3%, or no more than about 1% of the Linker Subunit L2s in a sample of conjugate, are cleaved when the conjugate is present in an extracellular environment (e.g., in plasma). Whether a Linker Subunit L2 is not substantially sensitive to the extracellular environment can be determined, for example, by incubating independently with plasma both (a) the conjugate (the “conjugate sample”) and (b) an equal molar amount of unconjugated Targeting unit or Drug unit (the “control sample”) for a predetermined time period (e.g., 2, 4, 8, 16, or 24 hours) and then comparing the amount of unconjugated Targeting unit or Drug unit present in the conjugate sample with that present in control sample, as measured, for example, by high performance liquid chromatography.

In some embodiments, a Linker or Linker Subunit L2 promotes cellular internalization. In some embodiments, a Linker or Linker Subunit L2 promotes cellular internalization when conjugated to the Drug unit such as a cytotoxic agent (i.e., in the milieu of the Linker-Drug unit moiety of a conjugate as described herein). In yet other embodiments, a Linker or Linker Subunit L2 promotes cellular internalization when conjugated to both the Drug unit and the Targeting unit (i.e., in the milieu of a conjugate as described herein).

A variety of Linker Subunits L2 that can be used with the present compositions and methods are described in, for example, WO 2004010957. In some embodiments, a Linker Subunit L2 includes a protease cleavable linker comprising a thiol-reactive spacer and a dipeptide (e.g., maleimidyl caproyl valine alanine). In some embodiments, a Linker Subunit L2 includes protease cleavable linker comprising a thiol-reactive maleimidocaproyl spacer, an amino acid or peptide and a self-immolative group. In some embodiments, a Linker Subunit L2 includes protease cleavable linker comprising a thiol-reactive maleimidocaproyl spacer, a valine-citrulline dipeptide, and a p-amino-benzyloxycarbonyl self immolative group.

In some embodiments, a Linker Subunit L2 includes an acid cleavable linker such as a hydrazine linker or a quaternary ammonium linker (see, e.g., WO2017/096311 and WO2016/040684.)

In some embodiments, a Linker Subunit L2 includes a self-stabilizing moiety comprising a maleimide group as described in WO2013/173337.

In some embodiments, a Linker Subunit L2 includes a hydrophilic linker, such as, for example, the hydrophilic peptides in WO2015/123679 and the sugar alcohol polymer-based linkers disclosed in WO2013/012961 and WO2019/213046.

In other embodiments, a Linker Subunit L2 may be made using a variety of bifunctional protein coupling agents such as N-succinimidyl-3-(2-pyridyldithio) propionate (SPDP), succinimidyl-4-(N-maleimidomethyl) cyclohexane-1-carboxyl (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) have been described in, for example WO94/11026.

In some embodiments, Linker Subunits L2, can be prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### Amino Acid (AA) Unit

The Linkers optionally include an Amino Acid unit (AA). When present in a Linker, an Amino Acid unit connects a Stretcher unit (L1) to a Linker subunit L2. When s of AA is 0, the Amino Acid unit is absent (e.g., in any of Formulae I to IV). In some embodiments, an Amino Acid unit includes from 0 to 12 subunits. Each subunit of the Amino Acid unit is selected from a natural or non-natural alpha, beta or gamma amino acid or a Polar unit, such as a Sugar unit (SU), or a Carboxyl unit or a PEG unit attached to a subunit of the Amino Acid unit.

In some embodiments, an Amino acid unit is an amino acid or a dipeptide, tripeptide, tetrapeptide, pentapeptide, hexapeptide, heptapeptide, octapeptide, nonapeptide, decapeptide, undecapeptide or dodecapeptide, in which one or more of the subunits is optionally modified to form a Polar unit, such as a Sugar unit, a PEG unit or a Carboxyl unit.

In some embodiments, the subunits of the Amino Acid unit are selected from glycine and/or L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and Polar units (including PEG units attached to glycine or an L-amino acid). In some embodiments, the subunits of the Amino Acid unit are selected from glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and Polar units. In some embodiments, the subunits of the Amino Acid unit are selected from glycine and/or a mixture of L-amino acids and D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine, and Polar units (including PEG units attached to glycine or an D-amino acid).

In some embodiments, the subunits of the Amino Acid unit are selected from glycine and/or natural L-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar unit, such as a Sugar unit, or a Carboxyl unit or a PEG unit attached to a glycine or a L-amino acid. In some embodiments, the subunits of the Amino Acid unit are selected from glycine and/or D-amino acids, such as arginine, glutamine, phenylalanine, tyrosine, tryptophan, lysine, alanine, histidine, serine, proline, glutamic acid, aspartic acid, threonine, cysteine, methionine, leucine, asparagine, isoleucine, and valine and at least one Polar unit, such as a Sugar unit, or a Carboxyl unit or a PEG unit attached to a glycine or a D-amino acid.

In some embodiments, a subunit of the Amino acid unit independently has the formula denoted below in the square brackets:

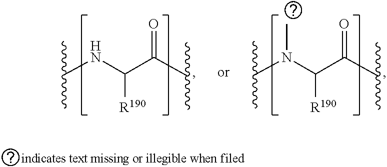

wherein R₁₉₀ is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂OOOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

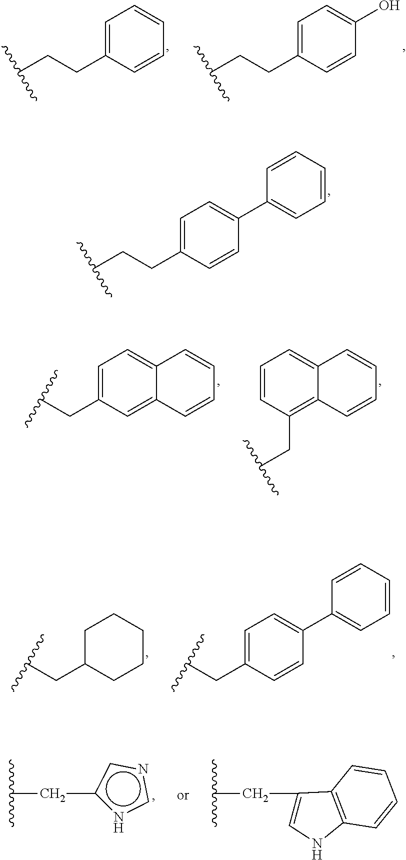

In some embodiments, each subunit of the Amino Acid unit is independently selected from the group consisting of the following L-(natural) amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar unit, such as a Sugar unit, or a Carboxyl unit or a PEG unit attached to a natural amino acid.

In some embodiments, a subunit of the Amino acid unit is not cysteine. In some embodiments, a subunit of the Amino Acid unit is not proline.

In some embodiments, each subunit of the Amino Acid unit is independently selected from the group consisting of the following D-isomers of these natural amino acids: alanine, arginine, aspartic acid, asparagine, histidine, glycine, glutamic acid, glutamine, phenylalanine, lysine, leucine, serine, tyrosine, threonine, isoleucine, tryptophan and valine; and at least one Polar unit, such as a Sugar unit, or a Carboxyl unit or a PEG unit attached to glycine or an L-amino acid.

In some embodiments, each subunit of the Amino Acid unit is independently selected from alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, p-alanine, aminoalkanoic acid, aminoalkynoic acid, amino alkanedioic acid, aminobenzoic acid, amino-heterocyclo-alkanoic acid, heterocyclo-carboxylic acid, citrulline, statine, diaminoalkanoic acid, and derivatives thereof; and at least one Polar unit, such as a Sugar unit, or a Carboxyl unit or a PEG unit attached to one of the subunits.

Illustrative of examples of alanine and derivatives thereof include but are not limited to: alanine (Ala), N-alkyl-alanine, dehydro-alanine, 4-thiazolylalanine, 2-pyridylalanine, 3-pyridylalanine, 4-pyridylalanine, β-(I-naphthyl)-alanine, β-(2-naphthyl)-alanine, α-aminobutyric acid, β-chloro-alanine, β-cyano-alanine, β-cyclopentyl-alanine, β-cyclohexyl-alanine, β-iodo-alanine, β-cyclopentenyl-alanine, β-tBu-alanine, β-cyclopropyl-alanine, β-diphenyl-alanine, β-fluoro-alanine, β-piperazinyl-alanine with the piperazine ring protected or not, β-(2-quinolyl)-alanine, β-(1,2,4-triazol-1-yi)-alanine, β-ureido-alanine, H-β-(3-benzothienyl)-Ala-OH, and H-β-(2-thienyl)-Ala-OH.

Illustrative of examples of arginine and derivatives thereof include but are not limited to: arginine (Arg), N-alkyl-arginine, H-Arg(Me)—OH, H—Arg(NH₂)—OH, H—Arg(NO₂)—OH, H-Arg(Ac)₂—OH, H—Arg(Me)₂—OH (asymmetrical), H-Arg(Me)₂—OH (symmetrical), 2-amino-4-(2′-hydroxyguanidino)-butyric acid (N-w-hydroxy-nor-arginine) and homoarginine.

Illustrative of examples of aspartic acid and derivatives thereof include but are not limited to: aspartic acid (Asp), N-alkyl-aspartic acid, and H-Asp(OtBu)—OH.

Illustrative of examples of asparagine and derivatives thereof include but are not limited to: asparagine (Asn), N-alkyl-asparagine, and isoasparagine (H-Asp-NH₂).

Illustrative of examples of cysteine (Cys) derivatives (containing no free SH group) thereof include but are not limited to: H-Cys(Acm)—OH, H—Cys(Trt)—OH, H—Cys(tBu)—OH, H-Cys(Bzl)—OH, H—Cys(Et)—OH, H—Cys(SO₃H)—OH, H—Cys(aminoethyl)-OH, H-Cys(carbamoyl)-OH, H—Cys(phenyl)—OH, H—Cys(Boc)—OH, and H-Cys(hydroxyethyl)—OH.

Illustrative of examples of histidine and derivatives thereof include but are not limited to: histidine (His), N-alkyl-histidine, H-His(Boc)—OH, H—His(Bzl)—OH, H—HBs(I-Me)—OH, H—His(I-Tos)—OH, H-2,5-diiodo-His-OH, and H-His(3-Me)—OH.

Illustrative of examples of glycine and derivatives thereof include but are not limited to: glycine (Gly), N-alkyl-glycine, H-propargylglycine

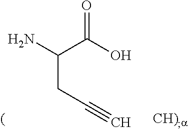

aminoglycine (protected or not), β-cyclopropyl-glycine, cyclopentyl-glycine, cyclohexyl-glycine, α-allylglycine, t-Butyl-glycine, neopentylglycine, and phenylglycine.

Illustrative of examples of glutamic acid and derivatives thereof include but are not limited to: glutamic acid (Glu), N-alkyl-glutamic acid, H-Glu(OtBu)—OH, H-γ-hydroxy-Glu-OH, H-γ-methylene-Glu-OH, H-γ-carboxy-Glu(OtBu)₂—OH, and pyroglutamic acid.

Illustrative of examples of glutamine and derivatives thereof include but are not limited to: glutamine (Gln), N-alkyl-glutamine, isoglutamine (H-Glu-NH₂), H-Gln(Trt)—OH, and H-Gln(isopropyl)—OH.

Illustrative of examples of phenylalanine and derivatives thereof include but are not limited to: phenylalanine (Phe), N-alkyl-phenylalanine, H-p-amino-Phe-OH, H-p-amino-Phe(Z)—OH, H-p-bromo-Phe-OH, H-p-Benzyl-Phe-OH, H-β-tBu-Phe-OH, H-p-carboxy-Phe(OtBu)—OH, H-p-carboxy-Phe-OH, H-β-cyano-Phe-OH, H-β-fluoro-Phe-OH, H-3,4-dichloro-Phe-OH, H-β-iodo-Phe-OH, H-p-nitro-Phe-OH, H-p-methyl-Phe-OH, H-pentafluoro-Phe-OH, H-m-fluoro-Phe-OH, H-a-Me-Phe-OH, H-4-phenyl-Phe-OH, homophenylalanine, chloro-phenylalanine and p-homophenylalanine.

Illustrative of examples of lysine and derivatives thereof include but are not limited to: lysine (Lys), N-alkyl-lysine, H-Lys(Boc)—OH, H-Lys(Ac)—OH, H-Lys(Formyl)—OH, H-Lys(Me)₂—OH, H-Lys(nicotinoyl)—OH, H-Lys(Me)₃—OH, H-trans-4,5-dehydro-Lys-OH, H-Lys(Aloc)—OH, H— H-b-hydroxy-Lys-OH, H-b-hydroxy-Lys(Boc)—OH, H-Lys(acetamidoyl)—OH, and H-Lys(isopropyl)—OH Illustrative of examples of leucine and derivatives thereof include but are not limited to: leucine (Leu), N-alkyl-leucine, 4,5-dehydroleucine, H-α-Me-Leu-OH, homoleucine, norleucine, and t-leucine.

Illustrative of examples of methionine and derivatives thereof include but are not limited to: methionine (Met), H-Met(O)—OH, and H-Met(O)₂—OH.

Illustrative of examples of serine and derivatives thereof include but are not limited to: serine (Ser), N-alkyl-serine, H-Ser(Ac)—OH, H—Ser(tBu)—OH, H—Ser(Bzl)—OH, H—Ser(β-chloro-Bzl)—OH, H-p-(3,4-dihydroxyphenyl)—Ser-OH, H-β-(2-thienyl)—Ser-OH, isoserine N-alkyl-isoserine, and 3-phenyliso serine.

Illustrative of examples of tyrosine and derivatives thereof include but are not limited to: tyrosine (Tyr), N-alkyl-tyrosine, H-3,5-dinitro-Tyr-OH, H-3-amino-Tyr-OH, H-3,5-dibromo-Tyr-OH, H-3,5-diiodo-Tyr-OH, H-Tyr(Me)—OH, H-Tyr(tBu)—OH, H-Tyr(Boc)—OH, H-Tyr(Bzl)-OH, H-Tyr(Et)—OH, H-3-iodo-Tyr-OH, and H-3-nitro-Tyr-OH.

Illustrative of examples of threonine and derivatives thereof include but are not limited to: threonine (Thr), N-alkyl-threonine, allo-threonine, H-Thr(Ac)—OH, H—Thr(tBu)—OH, and H-Thr(Bzl)—OH.

Illustrative of examples of isoleucine and derivatives thereof include but are not limited to: isoleucine (He), N-alkyl-isoleucine, allo-isoleucine, and norleucine.

Illustrative of examples of tryptophan and derivatives thereof include but are not limited to: tryptophan (Tip), N-alkyl-tryptophan, H-5-Me-Trp-OH, H-5-hydroxy-Trp-OH, H-4-Me-Trp-OH, H-α-Me-Trp-OH, H-Trp(Boc)—OH, H-Trp(Formyl)—OH, and H-Trp(Mesitylene-2-sulfonyl)—OH.

Illustrative of examples of proline and derivatives thereof include but are not limited to: proline (Pro), N-alkyl-proline, homoproline, thioproline, hydroxyproline (H-Hyp-OH), H-Hyp(tBu)—OH, H—Hyp(Bzl)—OH, H-3,4-dehydro-Pro-OH, 4-keto-proline, α-Me-Pro-OH, and H-4-fiuoro-Pro-OH.

Illustrative of examples of valine and derivatives thereof include but are not limited to: valine (Val), N-alkyl-valine, H-α-Me-Val-OH, and norvaline.

Illustrative of examples of ornithine and derivatives thereof include but are not limited to: ornithine, N-alkyl-ornithine, H-Orn(Boc)—OH, H—Om(Z)—OH, H-α-difluoro-Me-Orn-OH (Eflornitine), and H-Orn(Aloc)—OH.

Illustrative of examples of penicillamine and derivatives thereof include but are not limited to: penicillamine, H-penicillamme(Acm)—OH (H-β,β-dimethylcys(Acm)—OH) and N-alkyl-penicillamine.

Illustrative of examples of p-alanine and derivatives thereof include but are not limited to: p-alanine, N-alkyl-p-alanine, and dehydro-alanine.

Illustrative of examples of an aminoalkanoic acid and derivatives thereof include but are not limited to: N-alkylaminoalkanoic acid, aminobutyric acid, 4-(neopentyloxysulfonyl)-aminobutyric acid, ε-aminocaproic acid, α-aminoisobutyric acid, piperidylacetic acid, 3-amrnopropionic acid, 3-amino-3-(3-pyridyl)-propionic acid, and 5-aminopentanioic acid (amino valeric acid).

Illustrative of examples of an aminoalkynoic acid and derivatives thereof include but are not limited to: N-alkylaminoalkynoic acid, 6-amino-4-hexynoic acid, 6-(Boc-amino)-4-hexynoic acid.

Illustrative of examples of an aminoalkanedioic acid and derivatives thereof include but are not limited to: N-alkylaminoalkanedioic acid, 2-aminohexanedioic acid, 2-aminoheptanedioic acid, 2-aminooctanedioic acid (H-Asu-OH).

Illustrative of examples of an aminobenzoic acid and derivatives thereof include but are not limited to: N-alkylaminobenzoic acid, 2-aminobenzoic acid, 3-aminobenzoic acid, and 4-aminobenzoic acid.

Illustrative of examples of an amino-heterocyclo-alkanoic acid and derivatives thereof include but are not limited to: N-alkylamino-heterocyclo-alkanoic acids, 4-amino-1-methyl-IH-imidazol-2-carboxylic acid, 4-amino-1-methyl-IH-pyrrole-2-carboxylic acid, 4-amino-piperidine-4-carboxylic acid (H-Pip-OH; 1-protected or not), 3-amino-3-(3-pyridyl)-propionic acid.

Illustrative of examples of a heterocyclo-carboxylic acid and derivatives thereof include but are not limited to: azetidine-2-carboxylic acid, azetidine-3-carboxylic acid, piperidine-4-carboxylic acid, and thiazolidine-4-carboxylic acid.

Illustrative of examples of citrulline and derivatives thereof include but are not limited to: citrulline (cit), N-alkyl-citrulline, thio citrulline, S-methyl-thiocitrulline, and homocitrulline.

Illustrative of examples of statine and derivatives thereof include but are not limited to: statine, N-alkyl-statine, cyclohexylstatine, and phenylstatηie.

Illustrative of examples of diaminoalkanoic acid (Dab) and derivatives thereof include but are not limited to: N-alkyl-diamino-alkanoic acids, N,N-dialkylamino-alkanoic acids, α,γ-diaminobutyric acid (H-Dab-OH), H-Dab(Aloc)—OH, H-Dab(Boc)—OH, H-Dab(Z)—OH, α,β-diaminopropionic acid and its side-chain protected versions.

In some embodiments, an Amino Acid unit may be terminated with a capping group, such as a straight chain or branched alkyl group, or a polyethylene chain (from 1 to 30 subunits) or a PEG unit.

Exemplary embodiments of an Amino Acid unit include the following, wherein SU is a Sugar unit, PEG is a PEG unit and CU is a Carboxyl unit:

In some embodiments, an Amino Acid unit comprises SU.

In some embodiments, an Amino Acid unit comprises SU-Lys-SU.

In some embodiments, an Amino Acid unit comprises SU-Lys-SU-tert-butyl.

In some embodiments, an Amino Acid unit comprises SU-Lys.

In some embodiments, an Amino Acid unit comprises Lys-SU.

In some embodiments, an Amino Acid unit comprises Lys-SU-Lys(PEG).

In some embodiments, an Amino Acid unit comprises SU-Lys(PEG)—SU.

In some embodiments, an Amino Acid unit comprises SU-Glu-SU.

In some embodiments, an Amino Acid unit comprises Lys(PEG).

In some embodiments, an Amino Acid unit comprises Lys(PEG)-Lys(PEG)

In some embodiments, an Amino Acid unit comprises CU.

In some embodiments, an Amino Acid unit comprises CU-CU.

In some embodiments an Amino Acid Unit is present and is linked to a peptide of a Linker Subunit L2 via a peptide bond. In some embodiments, such an Amino Acid unit-Linker Subunit L2 comprises SU-Val-Cit-, wherein the wavy line indicates a bond to the remainder of Linker Subunit L2 or to a Drug unit. In some embodiments, such an Amino Acid unit—Linker Subunit L2 comprises SU-Val-Ala-, wherein the wavy line indicates a bond to the remainder of Linker Subunit L2 or to a Drug unit. In some embodiments, such an Amino Acid unit—Linker Subunit L2 comprises SU-Val-Lys-, wherein the wavy line indicates a bond to the remainder of Linker Subunit L2 or to a Drug unit. In some embodiments, such an Amino Acid unit—Linker Subunit L2 comprises SU-Gly-Gly-Phe-Gly (SEQ ID NO: 44), wherein the wavy line indicates a bond to the remainder of Linker Subunit L2 or to a Drug unit.

In some embodiments, such an Amino Acid unit—Linker Subunit L2 comprises Val-Lys(PEG)-, wherein the wavy line indicates a bond to the remainder of Linker Subunit L2 or to a Drug unit. In some embodiments, such an Amino Acid unit—Linker Subunit L2 comprises Val-Cit(PEG)-, wherein the wavy line indicates a bond to the remainder of Linker Subunit L2 or to a Drug unit. In some embodiments, such an Amino Acid unit-Linker Subunit L2 comprises Lys(PEG)-Val-Cit-, wherein the wavy line indicates a bond to the remainder of Linker Subunit L2 or to a Drug unit. In some embodiments, such an Amino Acid unit—Linker Subunit L2 comprises Lys(PEG)-Gly-Gly-Phe-Gly-(SEQ ID NO: 46), wherein the wavy line indicates a bond to the remainder of Linker Subunit L2 or to a Drug unit.

In some embodiments, such an Amino Acid unit—Linker Subunit L2 comprises CU-Val-Cit-, wherein the wavy line indicates a bond to the remainder of Linker Subunit L2 or to a Drug unit. In some embodiments, such an Amino Acid unit—Linker Subunit L2 comprises CU-Val-Lys-, wherein the wavy line indicates a bond to the remainder of Linker Subunit L2 or to a Drug unit. In some embodiments, such an Amino Acid unit—Linker Subunit L2 comprises CU-Val-Ala-, wherein the wavy line indicates a bond to the remainder of Linker Subunit L2 or to a Drug unit. In some embodiments, such an Amino Acid unit—Linker Subunit L2 comprises Val-CU-, wherein the wavy line indicates a bond to the remainder of Linker Subunit L2 or to a Drug unit, and wherein CU comprises a Lysine residue. In some embodiments, such an Amino Acid unit—Linker Subunit L2 comprises CU-Gly-Gly-Phe-Gly (SEQ ID NO: 45), wherein the wavy line indicates a bond to the remainder of Linker Subunit L2 or to a Drug unit.

In some embodiments, the Amino Acid unit is present and is attached to Linker Subunit L2 by a non-peptidic bond. In some embodiments, the Amino Acid unit is attached to Linker Subunit L2 by a peptidic linking group such as a C₁-C₁₀ alkylene, C₂-C₁₀ alkenylene, C₂-C₁₀ alkynylene, or polyethylene glycol.

In some embodiments, provided is a Linker intermediate or Linker, wherein L2 or AA-L 2 has one of the following structures:

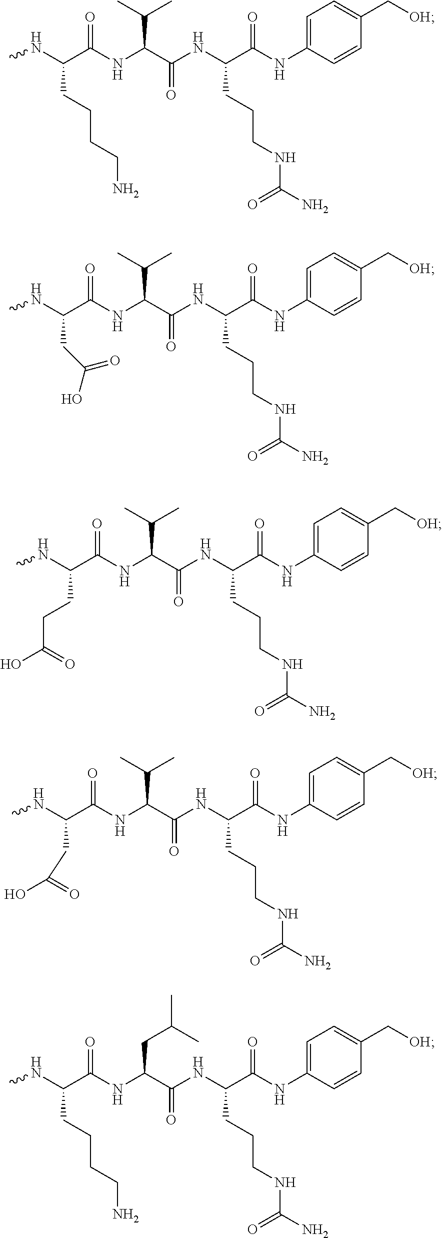

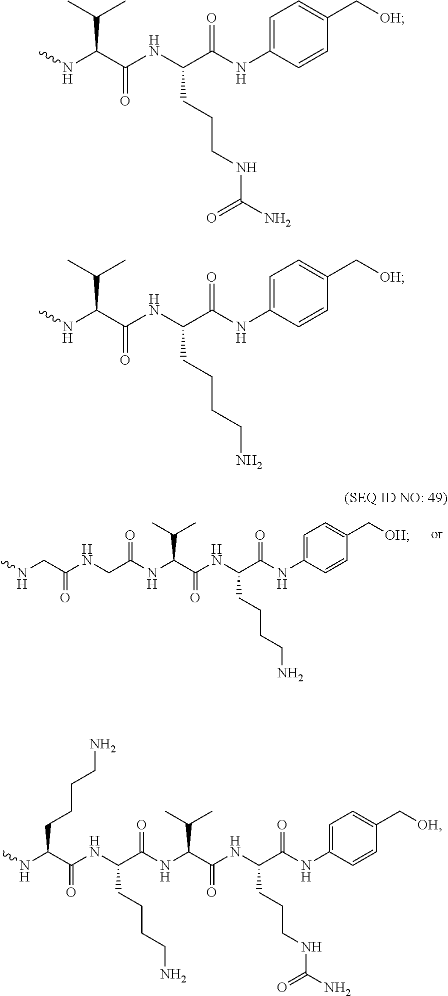

wherein the wavy line on the amino group indicates an attachment site for a Stretcher unit, and the Drug unit is attached to the benzyl alcohol.

### Stretcher Unit (1)

The Stretcher unit (L1) is capable of linking a Targeting unit to an Amino Acid unit (AA) or to a Linker Subunit L2. A Stretcher unit has a functional group that can form a bond with a functional group of a Targeting unit. In some embodiments of the Linker, the Stretcher unit is attached to an Amino Acid unit, which is attached to a Linker Subunit L2 (i.e., when s of AA is 1; see e.g., Formulae (I) to (IV)). In some embodiments, a Stretcher unit is attached to a Linker Subunit L2 (i.e., when s of AA is 0; see e.g., Formulae (I) to (IV)). In some embodiments, a Stretcher unit is attached to an Amino Acid unit-Linker Subunit L2 after the Amino Acid unit-Linker Subunit L2 is formed. In some embodiments, a Stretcher unit is attached to an Amino Acid unit-Linker Subunit L2-Drug unit after the Amino Acid unit-Linker Subunit L2-Drug unit is formed. In some embodiments, a Stretcher unit is attached to a Linker Subunit L2-Drug unit after the Linker Subunit L2-Drug unit is formed.

A functional group of the Stretcher unit for attachment to a Targeting unit may include, for example, maleimide, haloacetamide, sulfhydryl group, NHS ester, aldehyde, ketone, carbonyl, hydrazide, hydroxylamine, amine, amino, hydrazine, thiosemicarbazone, hydrazine carboxyl, or arylhydrazide.

Functional groups that can be present on a Targeting unit, either naturally or via chemical manipulation include, but are not limited to, sulfhydryl (—SH), amino, hydroxyl, carboxy, the anomeric hydroxyl group of a carbohydrate, and carboxyl groups. In one aspect, the Targeting unit's functional groups are sulfhydryl and amino. Sulfhydryl groups can be generated by reduction of an intramolecular disulfide bond of a Targeting unit. Alternatively, sulfhydryl groups can be generated by reaction of an amino group of a lysine moiety of a Targeting unit using 2-iminothiolane (Traut's reagent) or another sulfhydryl generating reagent.

In some embodiments, the Stretcher unit forms a bond with a sulfur atom of a Targeting unit via a maleimide group of the Stretcher unit. The sulfur atom can be derived from, for example, a sulfhydryl group of a Targeting unit (e.g., a thiol group of an interchain disulfide bond). Representative Stretcher units of this embodiment are depicted in the following Formulas 100 and 101, wherein L is a Targeting unit and the wavy line indicates an attachment site for an Amino Acid unit or to a Linker Subunit L2:

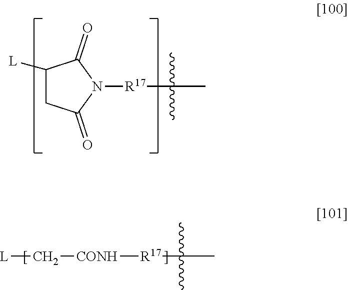

In some embodiments, provided is a Linker, wherein the Stretcher unit is selected from the following:

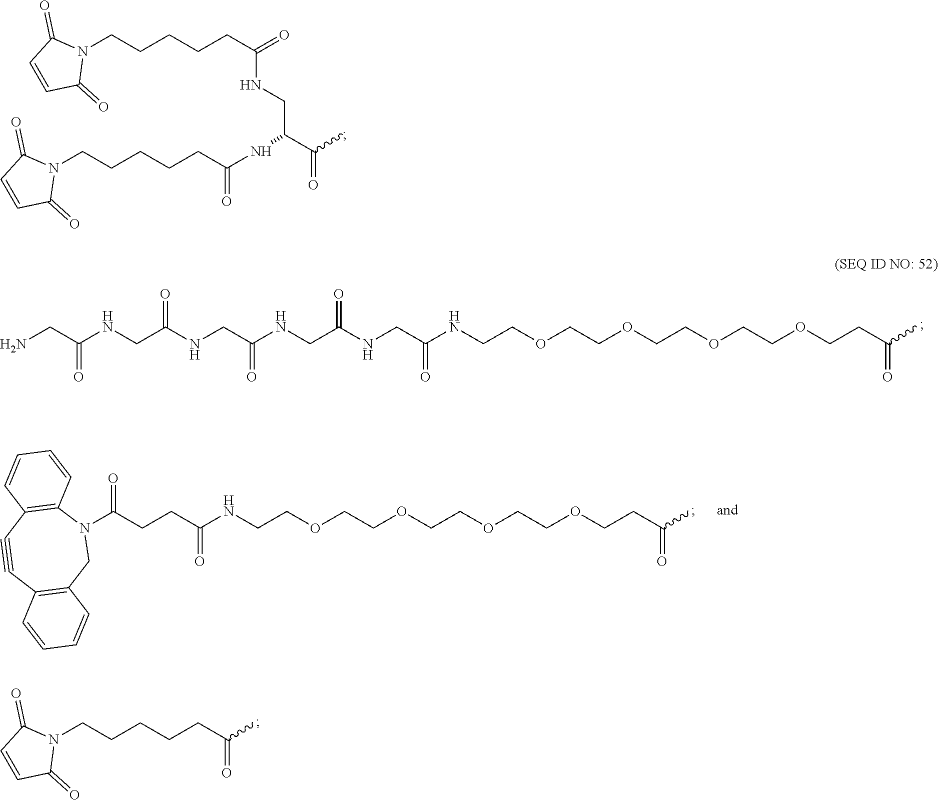

wherein the wavy line [symbol] indicates an attachment site of the Stretcher unit to an Amino Acid unit.

In formulas 100 and 101, R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene-(where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene-(where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —Cr—C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C,—C₈ alkylene-(CH₂—O—CH₂)_{b}—NH— (where b is 1 to 26), —(CH₂—O—CH₂)_{b}—C₁-C₈ alkylene-NH-(where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)—NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)—NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)—NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈ carbocyclo-S—, —O—(C₁-C₈ alkyl)—S—, -arylene-S—, —C₁-C₁₀ alkylene-arylene-S—, -arylene-C₁-C₁₀ alkylene-S—, —C₁-C₁₀ alkylene-(C₃-C₈ carbocyclo)—S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)—S—, or —(C₃-C₈ heterocyclo)-C₁-C₁₀ alkylene-S—. Any of the R¹⁷ substituents can be substituted or unsubstituted (also referred to as non-substituted). In some aspects, the R¹⁷ substituents are unsubstituted. In some aspects, the R¹⁷ substituents are optionally substituted. In some aspects, the R¹⁷ groups (see., e.g., WO2013/173337) such as, for example, —(CH₂)ₓNH₂, —(CH₂)ₓNHR^{a}, and —(CH₂)ₓNR^{a2}, wherein x is an integer of from 1-4 and each R^{a} is independently selected from the group consisting of C₁-C₆ alkyl and C₁-C₆ haloalkyl, or two R^{a} groups are combined with the nitrogen to which they are attached to form an azetidinyl, pyrrolidinyl or piperidinyl group.

In some embodiments of formula 100, R¹⁷ is —C₁-C₆ alkylene-C═O)—. In some embodiments, R¹⁷ is —C₁ alkylene-C(═O)—.

In some embodiments of formula 100, R¹⁷ is —(CH₂—O—CH₂)_{b}—C₁-C₈ alkylene-(where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene-(where b is 1 to 26), —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), or —C₁-C₈ alkylene-NH—(C(═O))—(CH₂—O—CH₂)_{b}—C₁-C₈ alkylene-NH— (where b is 1 to 26).

In other embodiments, the Stretcher unit is linked to the Targeting unit via a disulfide bond between a sulfur atom of the Stretcher unit and a sulfur atom of the Targeting unit. A representative Stretcher unit of this embodiment is depicted in the following Formula 102, wherein L is the Targeting unit, the wavy line indicates an attachment site for an Amino Acid unit or a Linker Subunit L2 and R¹⁷ is as described above for Formulae 100 and 101.

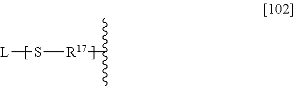

In yet another embodiment, a reactive group of a Stretcher unit contains a reactive site that can form a bond with a primary or secondary amino group of a Targeting unit. Examples of these reactive sites include, but are not limited to, activated esters such as succinimide esters, 4-nitrophenyl esters, pentafluorophenyl esters, tetrafluorophenyl esters, anhydrides, acid chlorides, sulfonyl chlorides, isocyanates and isothiocyanates. Representative Stretcher units of this embodiment are depicted in Formulas 103, 104, and 105, wherein L is a Targeting unit, the wavy line indicates an attachment site for an Amino Acid unit or a Linker Subunit L2 and R¹⁷ is as described above for Formula 100 and 101:

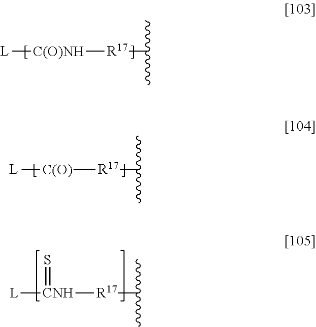

In yet another embodiment, a reactive group of a Stretcher unit contains a reactive site that is reactive to a modified carbohydrate's (—CHO) group that can be present on a Targeting unit. For example, a carbohydrate can be mildly oxidized using a reagent such as sodium periodate and the resulting (—CHO) unit of the oxidized carbohydrate can be condensed with a Stretcher unit that contains a functionality such as a hydrazide, an oxime, a primary or secondary amine, a hydrazine, a thiosemicarbazone, a hydrazine carboxyl, or an arylhydrazide (such as those described by Kaneko, T. et al. (1991) Bioconjugate Chem. 2:133-41.) Representative Stretcher units of this embodiment are depicted in the following Formulas 106, 107, and 108, wherein L is a Targeting unit, the wavy line indicates an attachment site for an Amino Acid unit or a Linker Subunit L2 and R¹⁷ is as described above for Formulae 100 and 101:

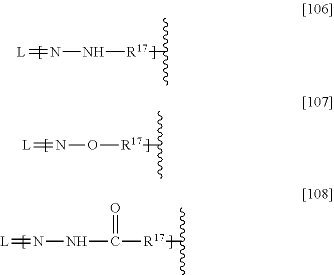

In some embodiments, it will be desirable to extend the length of a Stretcher unit. Accordingly, a Stretcher unit can comprise additional components. Representative Stretcher units of this embodiment are depicted in the following Formula 109, wherein L is a Targeting unit, the wavy line indicates an attachment site for an Amino Acid unit or a Linker Subunit L2 and R¹⁷ is as described above for Formula 100 and 101:

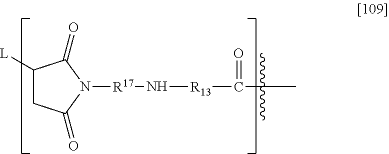

In some aspects of this embodiment, R¹⁷ is —C₁-C₅ alkylene-C(═O)—. R¹³ is —C₁-C₆ alkylene-, —(CH₂—O—CH₂)_{b}— (where b is 1 to 26), —C₃-C₈ carbocyclo-, -arylene-, —C₁-C₁₀ heteroalkylene-, —C₃-C₈ heterocyclo-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₁-C₁₀ alkylene-(C₃-C₈ heterocyclo)-, or —(C₃-C₈ heterocyclo)-C₁-C₁₀ alkylene-. In preferred embodiments, R¹³ is —(CH₂—O—CH₂)_{b}—, where b is 1 to 26.

### Targeting Units

In some embodiments, the Linkers are attached to Targeting units to form Targeting unit-Linkers. In some embodiments, the Linkers are attached to Targeting units via a Stretcher unit (L1) and to a Drug unit(s) via a Linker Subunit L2 to form a conjugate. In some embodiments, the Linkers are attached to a Targeting unit(s) via a Stretcher unit (L1) and to a Drug unit(s) via a Linker Subunit L2 for form a conjugate. The Targeting units can be antibodies, antigen binding portions thereof or non-antibody targeting units. Non-antibody targeting units may also be referred to as non-antibody scaffolds.

In some embodiments, a Targeting unit specifically binds to a target molecule. As used herein, “specifically binds” refers to the ability of a Targeting unit (e.g., an antibody or portion thereof) described herein to bind to a target with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the Targeting unit and the concentration of target polypeptide. The person of ordinary skill in the art can determine appropriate conditions under which the antibodies, antibody binding portions and non-antibody scaffolds described herein selectively bind to a target using any suitable methods, such as titration of a binding agent in a suitable cell binding assay. A Targeting unit specifically bound to its target is not displaced by a non-similar competitor. In certain embodiments, a Targeting uni is said to specifically bind to its target when it preferentially recognizes its target in a complex mixture of proteins and/or macromolecules.

As used herein, the term “antibody” refers to an immunoglobulin molecule and immunologically active portions of immunoglobulin molecules, i.e., molecules that contain an antigen binding site(s) that specifically bind(s) to a target antigen. The term generally refers to antibodies comprised of two immunoglobulin heavy chain variable regions and two immunoglobulin light chain variable regions including full length antibodies (having heavy and light chain constant regions).

Each heavy chain is typically composed of a variable region (abbreviated as a VH region) and a constant region. The heavy chain constant region may include three domains CH1, CH2 and CH3 and optionally a fourth domain, CH4. Each light chain is composed of a variable region (abbreviated as a VL region) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus includes three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of an antibody refers to the portions of an antibody having VH and/or VL sequences of an antibody or the CDRs of an antibody and that specifically binds to the target antigen. Examples of antigen binding portions include a Fab, a Fab′, a F(ab′)₂, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)₂ and Fv refer to the following: (i) a Fab is a monovalent fragment composed of the VL, VH, CL and CH₁ domains; (ii) a F(ab′)₂ is a bivalent fragment comprising two Fab fragments linked to one another in the hinge region via a disulfide bridge; and (iii) a Fv composed of the VL and VH domains. Although the two domains of the Fv fragment, namely VL and VH, are encoded by separate coding regions, they may further be linked to one another using a synthetic linker, e.g., a poly-G4S amino acid sequence (‘(G₄S)ₙ’ disclosed as SEQ ID NO: 1, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL regions are expressed on a single polypeptide chain, but using a linker connecting the VH and VL regions that is too short for the two regions to be able to combine on the same chain, thereby forcing the VH and VL regions to pair with complementary regions of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antigen binding portion of an antibody containing a single monomeric variable antibody region. Single domains antibodies can be derived from the variable region of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the Targeting unit is an antibody or antigen binding portion thereof is a bispecific or multispecific binding agent. Bispecific and multi-specific antibodies include the following: an scFv1—ScFv2, an ScFv1₂-Fc-scFv2₂, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

In some embodiments, the Targeting unit is a cancer associated antigen such as CD19, CD20, CD30, CD33, CD38, CA125, MUC-1, prostate-specific membrane antigen (PSMA), CD44 surface adhesion molecule, mesothelin (MLSN), carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, 1p19q, ABL1, AKT1, ALK, APC, AR, ATM, BRAF, BRCA1, BRCA2, cKIT, cMET, CSF1R, CTNNB1, FGFR1, FGFR2, FLT3, GNA11, GNAQ, GNAS, HRAS, IDH1, IDH2, JAK2, KDR (VEGFR2), KRAS, MGMT, MGMT-Me, MLH1, MPL, NOTCH1, NRAS, PDGFRA, Pgp, PIK3CA, PR, PTEN, RET, RRM1, SMO, SPARC, TLE3, TOP2A, TOPO1, TP53, TS, TUBB3, VHL, CDH1, ERBB4, FBXW7, HNF1A, JAK3, NPM1, PTPN11, RB1, SMAD4, SMARCB1, STK1, MLH1, MSH2, MSH6, PMS2, ROS1, ERCC1, 5T4 (TPBG), B7-H3, CCR7, CD105, CD22, CD46, CD47, CD56, CD70, CD71, CD79b, CDH6, CLDN6, CLDN18.2, CLEC12A, DLL3, DR5, ERBB3 (HER3), EPCAM, FOLR1, IGF1R, IL2RA (CD25), IL3RA, ITGB6, LIV-1, LRRC15, mesothelin (MSLN), NaPi2b (SLC34A2), nectin-4, PTK7, ROR1, SEZ6, SLC44A4, SLITRK6, Tissue Factor (TF), TROP2 or B7-H4. According to the invention, the terms “cancer associated antigen”, “tumor antigen”, “tumor expressed antigen”, “cancer antigen” “cancer associated antigen” and “cancer expressed antigen” are equivalents and are used interchangeably herein.

In some embodiments, a Targeting unit specifically binds to a target such as CD19, CD20, CD30, CD33, CD70, LIV-1 or EGFRv3.

In some embodiments, the Targeting unit is an antibody (or fragment thereof) that binds to a target having a sequences as disclosed in Leuschner et al., US 2022/0048951 and/or Lerchen et al., US 2022/0016258. Non-limiting examples of monoclonal antibodies include rituximab (Rituxan®), trastuzumab (Herceptin®), pertuzumab (Perjeta®)), bevacizumab (Avastin®), ranibizumab (Lucentis®), cetuximab (Erbitux®), alemtuzumab (Campath®), panitumumab (Vectibix®), ibritumomab (Zevalin®), tositumomab (Bexxar®), ipilimumab, zalutumumab, dalotuzumab, figitumumab, ramucirumab, galiximab, farletuzumab, ocrelizumab, ofatumumab (Arzerra®), the CD20 antibodies 2F2 (HuMax-CD20), 7D8, IgM2C6, IgG1 2C6, 11 B8, B1, 2H7, LT20, 1 FS or AT80 (see Teeling et al., J. Immunol. 177:362-371 (2006)),daclizumab (Zenapax®), and anti-LHRH receptor antibodies such as clones A9E4, F1G4, AT2G7, GNRH03, GNRHR2, etc. which can be used in combination with, inter alia, a conjugate in accordance with the invention.

In some embodiments, provided are FOLR1 antibodies, antigen binding portions thereof and other binding agents as well as conjugates of such antibodies, antigen binding portions and other binding agents. Also provided are methods of using the FOLR1 antibodies, antigen binding portions and other binding agents and conjugates thereof for the treatment of cancer and other diseases. The invention disclosed herein is based in part on FOLR1 antibodies, antigen-binding portions thereof and other binding agents as well as conjugates thereof that specifically bind to FOLR1 and that exhibit improved properties. FOLR1 is an important and advantageous therapeutic target for the treatment of certain cancers. The FOLR1 antibodies, antigen binding portions thereof, other binding agents and conjugates thereof provide compositions and methods based on the use of such antibodies, antigen binding portions and related binding agents, and conjugates thereof, in the treatment of FOLR1+ cancers and other diseases.

In some embodiments, a Targeting unit is a non-antibody scaffold. In some embodiments, a Targeting unit is a non-antibody protein scaffold. Such non-antibody scaffolds include, for example, Affibodies, Affilins, Anticalins, Atrimers, Avimers, Bicyclic peptides, Cys-knots, DARPins, FN3 scaffolds (e.g., Adnectins, Centyrins, Pronectins, and Tn3), Fynomers, Kunitz domains and OBodies. (See, e.g., Vazquez-Lombardi et al., Drug Discovery Today 20(10):1271 (2015) and the references cited therein.) Such Non-antibody protein scaffolds include, for example, Affibodies, Affilins, Anticalins, Atrimers, Avimers, Bicyclic peptides, Cys-knots, DARPins, FN3 scaffolds (e.g., Adnectins, Centyrins, Pronectins, and Tn3), Fynomers, Kunitz domains and OBodies. (See, e.g., Vazquez-Lombardi et al., Drug Discovery Today 20(10):1271 (2015) and the references cited therein.) Non-antibody scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynorners, Kunitz domains, pronectins and OBodies. Peptide-sized non-antibody scaffolds include, for example, avimers, bicyclic peptides and cysteine knots. Non-antibody protein scaffolds can be considered to fall into two structural categories, domain-sized constructs (in the range of 6 to 20 kDa), and constrained peptides (in the 2-4 kDa range). Domain-sized non-antibody scaffolds include, but are not limited to, affibodies, affilins, anticalins, atrimers, DARPins, FN3 scaffolds (such as adnectins and centyrins), fynomers, Kunitz domains, pronectins and OBodies. Peptide-sized non-antibody scaffolds include, for example, avimers, bicyclic peptides and cysteine knots. These non-antibody scaffolds and the underlying proteins or peptides on which they are based or from which they have been derived are reviewed by, e.g. Simeon and Chen, Protein Cell 9(1): 3-14 (2018); Vazquez-Lombardi et al., Drug Discovery Today 20: 1271-1283 (2015), and by Binz et al., Nature Biotechnol. 23: 1257-1268 (2005), the contents of each of which are herein incorporated by reference in their entireties.

Advantages of using non-antibody scaffolds include increased affinity, target neutralization, and stability. Various non-antibody scaffolds also can overcome some of the limitations of antibody scaffolds, e.g., in terms of tissue penetration, smaller size, and thermostability. Some non-antibody scaffolds can also permit easier construction, not being hindered, for example, by potential light chain association concerns when bispecific constructs are desired. Methods of constructing constructs on a non-antibody scaffold are known to those of ordinary skill in the art.

Accordingly, in some embodiments, a Targeting unit can comprise a non-antibody scaffold. Accordingly, in some embodiments, a Targeting unit can comprise a non-antibody scaffold protein. One of skill in the art would appreciate that a Targeting unit can include, in some embodiments, e.g., an adnectin scaffold or a portion derived from human tenth fibronectin type III domain (10Fn3); an anticalin scaffold derived from human lipocalin (e.g., such as those described in, e.g., WO2015/104406); an avimer scaffold or a protein fragment derived from the A-domain of low density-related protein (LRP) and/or very low density lipoprotein receptor (VLDLR); a fynomer scaffold or portion of the SH3 domain of FYN tyrosine kinase; a kunitz domain scaffold or portion of Kunitz-type protease inhibitors, such as a human trypsin inhibitor, aprotinin (bovine pancreatic trypsin inhibitor), Alzheimer's amyloid precursor protein, and tissue factor pathway inhibitor; a knottin scaffold (cysteine knot miniproteins), such as one based on a trypsin inhibitor from E. elaterium; an affibody scaffold or all or part of the Z domain of S. aureus protein A; a β-Hairpin mimetic scaffold; a Designed ankyrin repeat protein (DARPin) scaffold or artificial protein scaffolds based on ankyrin repeat (AR) proteins; or any scaffold derived or based on human transferrin, human CTLA-4, human crystallin, and human ubiquitin. For example, the binding site of human transferrin for human transferrin receptor can be diversified to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is, and in accordance with the methods described herein, against a target antigen of interest to identify those variants having optimal selectivity and affinity for the target antigen. See, e.g., Hey et al. (2005) TRENDS Biotechnol. 23(10):514⁻⁵²². FOLR1 Targeting Units

In some embodiments, a Targeting agent is an anti-FOLR1 antibody or an antigen binding portion thereof that specifically binds to FOLR1. In some embodiments provided are conjugates of such antibodies and antigen binding portions thereof. Conjugates comprising Targeting agents that specifically bind to FOLR1 are useful in methods for the treatment of cancer and other diseases. Such conjugates of FOLR1 antibodies and antigen-binding portions thereof exhibit improved properties as compared to other FOLR1 conjugates when attached to the linker-drugs described herein. FOLR1 is an important and advantageous therapeutic target for the treatment of certain cancers. The FOLR1 conjugates thereof provide compositions and methods based on the use of such conjugates n the treatment of FOLR1+ cancers and other diseases.

In some embodiments, a FOLR1 Targeting agent comprises a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having amino acids sequences selected from the sets of amino acid sequences set forth in the group consisting of: SEQ ID NO:30, SEQ ID NO:31, SEQ ID NO:32, SEQ ID NO:33, SEQ ID NO:34 and SEQ ID NO:35, respectively; and SEQ ID NO:36, SEQ ID NO:31, SEQ ID NO:37, SEQ ID NO:38, SEQ ID NO:39 and SEQ ID NO:40, respectively. In some embodiments, the VH and VL CDRs have the amino acids sequences set forth in SEQ ID NO:30, SEQ ID NO:31, SEQ ID NO:32, SEQ ID NO:33, SEQ ID NO:34 and SEQ ID NO:35, respectively. In some embodiments, the framework regions are human framework regions.

In some embodiments, the VH and VL regions have amino acid sequences that are selected from the pairs of amino acid sequences set forth in the group consisting of: SEQ ID NO:6 and SEQ ID NO:7, respectively; SEQ ID NO:8 and SEQ ID NO:9, respectively; SEQ ID NO:10 and SEQ ID NO:11, respectively; SEQ ID NO:12 and SEQ ID NO:13, respectively; SEQ ID NO:14 and SEQ ID NO:15, respectively; SEQ ID NO:16 and SEQ ID NO:17; respectively; SEQ ID NO:18 and SEQ ID NO:19; respectively; SEQ ID NO:20 and SEQ ID NO:21; respectively; SEQ ID NO:22 and SEQ ID NO:23; respectively; SEQ ID NO:24 and SEQ ID NO:25; respectively; SEQ ID NO:26 and SEQ ID NO:27; respectively; and SEQ ID NO:28 and SEQ ID NO:29; respectively; wherein the heavy and light chain framework regions are optionally modified with from 1 to 8 amino acid substitutions, deletions or insertions in the framework regions.

In some embodiments, the VH and VL regions have amino acid sequences that are selected from the pairs of amino acid sequences set forth in the group consisting of: SEQ ID NO:6 and SEQ ID NO:7, respectively; SEQ ID NO:8 and SEQ ID NO:9, respectively; SEQ ID NO:10 and SEQ ID NO:11, respectively; SEQ ID NO:12 and SEQ ID NO:13, respectively; SEQ ID NO:14 and SEQ ID NO:15, respectively; SEQ ID NO:16 and SEQ ID NO:17; respectively; SEQ ID NO:18 and SEQ ID NO:19; respectively; SEQ ID NO:20 and SEQ ID NO:21; respectively; SEQ ID NO:22 and SEQ ID NO:23; respectively; SEQ ID NO:24 and SEQ ID NO:25; respectively; SEQ ID NO:26 and SEQ ID NO:27; respectively; and SEQ ID NO:28 and SEQ ID NO:29; respectively.

In some embodiments, the VH and VL regions have amino acid sequences that are selected from the pairs of amino acid sequences set forth in the group consisting of: SEQ ID NO:8 and SEQ ID NO:9, respectively; SEQ ID NO:12 and SEQ ID NO:13, respectively; SEQ ID NO:14 and SEQ ID NO:15, respectively; SEQ ID NO:16 and SEQ ID NO:17; respectively; SEQ ID NO:20 and SEQ ID NO:21; respectively; SEQ ID NO:22 and SEQ ID NO:23; respectively; SEQ ID NO:24 and SEQ ID NO:25; respectively; and SEQ ID NO:26 and SEQ ID NO:27; respectively.

In some embodiments, the VH and VL regions have amino acid sequences that are selected from the pairs of amino acid sequences set forth in the group consisting of: SEQ ID NO:8 and SEQ ID NO:9, respectively; SEQ ID NO:12 and SEQ ID NO:13, respectively; and SEQ ID NO:26 and SEQ ID NO:27; respectively. In some embodiments, the VH and VL regions have amino acid sequences that are set forth in SEQ ID NO:8 and SEQ ID NO:9, respectively. In some embodiments, the VH and VL regions have amino acid sequences that are set forth in SEQ ID NO:12 and SEQ ID NO:13, respectively. In some embodiments, the VH and VL regions have amino acid sequences that are set forth in SEQ ID NO:26 and SEQ ID NO:27, respectively.

In some embodiments, the heavy chain variable region further comprises a heavy chain constant region. In some embodiments, the heavy chain constant region is of the IgG isotype. In some embodiments, the heavy chain constant region is an IgG1 constant region. In some embodiments, the IgG1 constant region has the amino acid sequence set forth in SEQ ID NO:41. In some embodiments, the heavy chain constant region is an IgG4 constant region. In some embodiments, the heavy chain constant region further comprises at least amino acid modification that decreases binding affinity to human FcgammaRIll. In some embodiments, the light chain variable region further comprises a light chain constant region. In some embodiments, the light chain constant region is of the kappa isotype. In some embodiments, the light chain constant region has the amino acid sequence set forth in SEQ ID NO:42.

In some embodiments, a FOLR1 conjugate is mono-specific. In some embodiments, a FOLR1 conjugate is bivalent. In some embodiments, a FOLR1 conjugate is bispecific.

In some embodiments, a FOLR1 conjugate comprises a Targeting unit that is an antibody comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR and LCDR3 disposed in light chain variable region framework regions, the VH and VL CDRs having amino acids sequences selected from the sets of amino acid sequences set forth in the group consisting of: (a) SEQ ID NO:30, SEQ ID NO:31, SEQ ID NO:32, SEQ ID NO:33, SEQ ID NO:34 and SEQ ID NO:35, respectively; and (b) SEQ ID NO:36, SEQ ID NO:31, SEQ ID NO:37, SEQ ID NO:38, SEQ ID NO:39 and SEQ ID NO:40, respectively. In certain embodiments, the VH and VL regions have amino acid sequences that are selected from the pairs of amino acid sequences set forth in the group consisting of: SEQ ID NO:26 and SEQ ID NO:27; respectively; and wherein the heavy and light chain framework regions are optionally modified with from 1 to 8 amino acid substitutions, deletions or insertions in the framework regions. In a specific embodiment, the Targeting unit is antibody F131 (VH SEQ ID NO: 26 and VL SEQ ID NO: 27). In a specific embodiment, the antibody is F131 and the Drug-Linker is LD038.

### Constant Regions

In some embodiments, a Targeting unit, such as an antibody or antigen-binding portion thereof or other Targeting unit, has an antibody constant region(s). In some embodiments, the constant region is a fully human constant region(s). In some embodiments, the constant region is a humanized constant region(s). In some embodiments, the constant region is a non-human constant region(s). An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgAl, and IgA2. The heavy-chain constant regions (Fc) that correspond to the different classes of immunoglobulins can be α, δ, ε, γ, and μ, respectively. The light chains can be one of either kappa (or K) and lambda (or A).

In some embodiments, a constant region can have an IgG isotype. In some embodiments, a constant region can have an IgG1 isotype. In some embodiments, a constant region can have an IgG2 isotype. In some embodiments, a constant region can have an IgG3 isotype. In some embodiments, a constant region can have an IgG4 isotype. In some embodiments, a constant region can have a hybrid isotype comprising constant regions from two or more isotypes. In some embodiments, an immunoglobulin constant region can be an IgG1 or IgG4 constant region. In some embodiments, a constant region is of the IgG1 isotype and has the amino acid sequence set forth in SEQ ID NO:2. In some embodiments, a constant region is of the kappa isotype and has the amino acid sequence set forth in SEQ ID NO:3.

Furthermore, a Targeting unit comprising an antibody or an antigen-binding portion thereof or non-antibody scaffold may be part of a larger molecule formed by covalent or noncovalent association of the antibody or antigen binding portion with one or more other proteins or peptides. Relevant to such Targeting units are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (‘hexahistidinyl tag’ disclosed as SEQ ID NO: 4) in order to produce bivalent and biotinylated scFv molecules (Kipriyanov, S. M., et al. (1994) Mol. Immunol. 31:10471058).

### Fc Domain Modifications to Alter Effector Function

In some embodiments, an Fc region or Fc domain of a Targeting unit, such as an antibody or antigen binding portion thereof or non-antibody scaffold, has substantially no binding to at least one Fc receptor selected from FcyRI (CD64), FcyRIIA (CD32a), FcyRIIB (CD32b), FcyRIIIA (CD16a), and FcyRIIIB (CD16b). In some embodiments, an Fc region or domain exhibits substantially no binding to any of the Fc receptors selected from FcyRI (CD64), FcyRIIA (CD32a), FcyRIIB (CD32b), FcyRIIIA (CD16a), and FcyRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcgamma receptor binding (hence likely lacking ADCC activity). The primary cells for mediating ADCC, NK cells, express FcgammaRIII only, whereas monocytes express FcgammaRI, FcgammaRII and FcgammaRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059⁻⁷⁰⁶³ (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C₃c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A Targeting unit, such as an antibody or antigen binding portion thereof or non-antibody scaffold, with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A Targeting unit, such as an antibody or antigen binding portion thereof or non-antibody scaffold, with diminished binding to FcRs can be prepared containing such amino acid modifications.

In some embodiments, a Targeting unit, such as an antibody or antigen binding portion thereof or non-antibody scaffold, comprises an Fc domain or region with one or more amino acid substitutions which diminish FcgammaR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies and Antigen Binding Portions and Other Targeting Units

In various embodiments, Targeting units such as antibodies and antigen binding portions thereof, can be produced in human, murine or other animal-derived cells lines. Recombinant DNA expression can be used to produce antibodies and antigen binding portions thereof. This allows the production of antibodies as well as a spectrum of antigen binding portions (including fusion proteins) in a host species of choice. The production of antibodies and antigen binding portions thereof in bacteria, yeast, transgenic animals and chicken eggs are also alternatives for cell-based production systems. The main advantages of transgenic animals are potential high yields from renewable sources.

Nucleic acid molecules encoding the amino acid sequence(s) of Targeting unit, such as an antibody or antigen binding portion thereof can be prepared by a variety of methods known in the art. These methods include, but are not limited to, preparation of synthetic nucleotide sequences encoding of an antibody or antigen binding portion. In addition, oligonucleotide-mediated (or site-directed) mutagenesis, PCR-mediated mutagenesis, and cassette mutagenesis can be used to prepare nucleotide sequences encoding an antibody or antigen binding portion. A nucleic acid sequence encoding at least an antibody or antigen binding portion thereof, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, N Y, 1982 and 1989), and Ausubel et al., Current Protocols in Molecular Biology (John Wiley & Sons), 1987-1993, and can be used to construct nucleic acid sequences and vectors that encode an antibody or antigen binding portion thereof or a VH or VL polypeptide thereof.

As used herein, the terms “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., an antibody or antigen binding portion thereof) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a Targeting unit, such as an antibody or antigen-binding portion thereof, can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but other mammalian cells may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or antigen binding portion thereof as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant antibodies or antigen-binding portions thereof. Known glycolytic genes can also provide very efficient transcriptional control signals. For example, the promoter and terminator signals of the phosphoglycerate kinase gene can be utilized.

Production of antibodies or antigen-binding portions in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding an antibody or antigen binding portion thereof or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding antibodies or antigen-binding portions thereof include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the variable region(s) or antigen binding portions thereof are then transfected singly with nucleotides encoding an antibody or an antibody polypeptide or antigen-binding portion thereof, or are co-transfected with a polynucleotide(s) encoding VH and VL chain coding regions. The transfected recipient cells are cultured under conditions that permit expression of the incorporated coding regions and the expressed antibody chains or intact antibodies or antigen binding portions are recovered from the culture.

In some embodiments, the nucleic acids containing the coding regions encoding an antibody or antigen-binding portion thereof are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of antibodies or antigen binding portions thereof, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding an antibody or antigen-binding portion thereof can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment, as known to one of ordinary skill in the art. (See, e.g., Johnston et al., 240 Science 1538 (1988)).

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled antibodies and antigen binding portions thereof. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of antibodies and antigen binding portions thereof in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more antibodies or antigen-binding portions thereof can be produced in vivo in an animal that has been engineered or transfected with one or more nucleic acid molecules encoding the polypeptides, according to any suitable method.

In some embodiments, an antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695⁻⁷¹³ (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H₂L₂ antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies or antigen-binding portions thereof via either route could be transfected with plasmids encoding additional copies of peptides, VH, VL, or VH plus VL chains in conjunction with additional selectable markers to generate cell lines with enhanced properties, such as higher production of assembled antibodies or antigen binding portions thereof or enhanced stability of the transfected cell lines.

Additionally, plants have emerged as a convenient, safe and economical alternative expression system for recombinant antibody production, which are based on large scale culture of microbes or animal cells. Antibodies or antigen binding portions thereof can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). An antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to the target antigen. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, an antigen binding portion is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be Targeting units (see, e.g. US 2006/0025576A1).

In some embodiments, the Targeting units comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the Targeting unit a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a Targeting unit is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a Targeting unit is a bispecific antibody is composed of a single polypeptide chain comprising two single chain FV portions (scFV) fused to each other by a peptide linker.

In some embodiments, a Targeting unit is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

Igg-like dual-variable domain antibodies (DVD-Ig) have been described by Wu et al., 2007, Nat Biotechnol 25:1290⁻⁹⁷; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure antibodies or antigen binding portions thereof of at least about 90% to 95% homogeneity are advantageous, as are those with 98% to 99% or more homogeneity, particularly for pharmaceutical uses. Once purified, partially or to homogeneity as desired, an intact antibody or antigen binding portions thereof can then be used therapeutically or in developing and performing assay procedures, immunofluorescent staining, and the like. See generally, Vols. I & II Immunol. Meth. (Lefkovits & Pernis, eds., Acad. Press, N Y, 1979 and 1981).

### Drug Units

In some embodiments, the Linkers are attached to a Drug unit(s), a Targeting unit and/or to a Targeting unit and to a Drug unit(s) (the latter also referred to as a conjugate, ADC or antibody drug conjugate). In some embodiments, a Linker via a Linker Subunit L2, is attached to at least one Drug unit. As used herein, in the context of a conjugate, the term “Drug unit” or drug refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

### Cytotoxic Agents

In some embodiments, a Drug unit is a cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52. Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9—SH (U.S. Pat. No. 4,424,219) (prepared by the reaction of maytansinol with H₂S or P₂S5); C-14-alkoxymethyl(demethoxy/CH₂OR) (see, U.S. Pat. No. 4,331,598); C-14-hydroxymethyl or acyloxymethyl (CH₂OH or CH₂OAc) (see, U.S. Pat. No. 4,450,254) (prepared from Nocardia); C-15-hydroxy/acyloxy (see, U.S. Pat. No. 4,364,866) (prepared by the conversion of maytansinol by Streptomyces); C-15-methoxy (see, U.S. Pat. Nos. 4,313,946 and 4,315,929) (isolated from Trewia nudiflora); C-18—N-demethyl (see, U.S. Pat. Nos. 4,362,663 and 4,322,348) (prepared by the demethylation of maytansinol by Streptomyces); and 4,5-deoxy (see, U.S. Pat. No. 4,371,533) (prepared by the titanium trichloride/LAH reduction of maytansinol).

Hemiasterlins include but are not limited to, hemiasterlin and HTI-286.

Other tubulin disrupting agents include taccalonolide A, taccalonolide B, taccalonolide AF, taccalonolide AJ, taccalonolide AI-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a Drug unit is an immune modulatory agent. An immune modulatory agent can be, for example, a TLR7 and/or TLR8 agonist, a STING agonist, a RIG-1 agonist or other immune modulatory agent.

In some embodiments, a Drug unit is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164, US 20150299194, US20110098248, US20100143301, and US20090047249.

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755, WO2017216054, WO2017190669, WO2017202704, WO2017202703, WO20170071944, US20140045849, US20140073642, WO2014056953, WO2014076221, WO2014128189, US20140350031, WO2014023813, US20080234251, US20080306050, US20100029585, US20110092485, US20110118235, US20120082658, US20120219615, US20140066432, US20140088085, US20140275167, and US20130251673, WO2018198091, and US20170131421.

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-1 agonist. Examples of RIG-1 agonists include KIN1148, SB-9200, KIN700, KIN600, KIN500, KIN100, KIN101, KIN400 and KIN2000.

### Toxins

In some embodiments, a Drug unit is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a Drug unit is a radioactive atom. A variety of radioactive isotopes are available for the production of radioconjugates. Examples include yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

### PROTACs

In some embodiments, a Drug unit is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Ligands

In some embodiments, a Drug unit includes ligands that can be bound by a Carboxyl unit, such as platinum (Pt), ruthenium (Ru), rhodium (Rh), gold (Au), silver (Ag), copper (Cu), molybdenum (Mo), titanium (Ti), or iridum (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

### Drug Loading

Conjugates can contain one or more Drug unit per Targeting unit. The number of Drug units per Targeting unit is referred to as drug loading. The drug loading of a Conjugate is represented by p^{load}, the average number of Drug units (drug molecules (e.g., cytotoxic agents)) per Targeting units (e.g., an antibody or antigen binding portion or non-antibody scaffold or non-antibody protein) in a conjugate. For example, if p^{load} is about 4, the average drug loading taking into account all of the Targeting units (e.g., antibodies or antigen binding portion or non-antibody scaffold or non-antibody proteins) present in the composition is about 4. In some embodiments, pload ranges from about 3 to about 5, from about 3.6 to about 4.4, or from about 3.8 to about 4.2. In some embodiments, p^{load} can be about 3, about 4, or about 5. In some embodiments, p^{load} ranges from about 6 to about 8, more preferably from about 7.5 to about 8.4. In some embodiments, p^{load} can be about 6, about 7, or about 8. In some embodiments, p^{load} ranges from about 8 to about 16.

The average number of Drug units per Targeting unit (e.g., antibody or antigen binding portion or non-antibody scaffold) in a preparation may be characterized by conventional means such as UV, mass spectroscopy, Capillary Electrophoresis (CE), and HPLC. The quantitative distribution of conjugates in terms of p^{load} may also be determined. In some instances, separation, purification, and characterization of homogeneous conjugates where p^{load} is a certain value from conjugates with other drug loadings may be achieved by means such as reverse phase HPLC or Hydrophobic Interaction Chromatography (HIC) HPLC.

### Exemplary Linkers and Linker Unit-Drug Unit Combinations

In some embodiments, a Linker intermediate ˜L1-AA≈ has the following general formula:

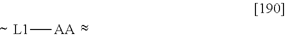

or a salt thereof, wherein AA is an Amino Acid unit having from one to 12 subunits selected from alpha, beta and gamma amino acids and derivatives thereof, Sugars units, Carboxyl units and amino acid subunits, optionally substituted with at least one PEG unit, provided that the Amino Acid unit comprises at least one Sugar unit, PEG unit or Carboxyl unit; L1 is a Stretcher unit; and the wavy line (˜) indicates an attachment site for a Targeting unit and the double wavy (≈) line indicates an attachment site for a Linker Subunit L2. In some embodiments, the Amino Acid unit comprises at least one Sugar unit, PEG unit, Carboxyl unit or a combination thereof;

In some embodiments, a Linker intermediate ˜AA-L2≈ has the following general formula:

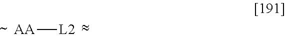

or a salt thereof, wherein AA is an Amino Acid unit having from one to 12 subunits selected from alpha, beta and gamma amino acids and derivatives thereof, Sugar units Carboxyl units and amino acid subunits optionally substituted with at least one PEG unit; L2 is a Linker Subunit optionally substituted with at least one Sugar unit, PEG unit, Carboxyl unit or a combination thereof; the wavy line (˜) indicates an attachment site for a Stretcher unit; and the double wavy (≈) line indicates an attachment site for a Drug unit; provide that ˜AA-L2≈comprises at least one Sugar unit, PEG unit, Carboxyl unit or a combination thereof.

In some embodiments, a Drug Linker intermediate ˜AA-L2-D has the following general formula:

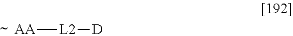

or a salt thereof, wherein AA is an Amino Acid unit having from one to 12 subunits selected from alpha, beta and gamma amino acids and derivatives thereof, Sugar units, Carboxyl units and amino acid subunits optionally substituted with at least one PEG unit; L2 is a Linker Subunit optionally substituted with at least one Sugar unit, PEG unit, Carboxyl unit or a combination thereof; D is a Drug unit; and the wavy line (˜) indicates an attachment site for a Stretcher unit, provided that ˜AA-L2-comprises at least one Sugar unit, PEG unit, Carboxyl unit, or combination thereof.

In some embodiments, a Linker ˜-L1-AA-L2≈ has the following general formula:

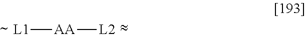

or a salt thereof, wherein L1 is a Stretcher unit; AA is an Amino Acid unit having from one to 12 subunits selected from alpha, beta and gamma amino acids and derivatives thereof, Sugar units, Carboxyl units and amino acid subunits optionally substituted with at least one PEG unit; L2 is a Linker Subunit optionally substituted with at least one Sugar unit, PEG unit, Carboxyl unit or a combination thereof; the wavy line (˜) indicates an attachment site for a Targeting unit, the double wavy (≈) line indicates an attachment site for a Drug unit; provided that ˜AA-L2≈comprises at least one Sugar unit, PEG unit, Carboxyl unit, or combination thereof. In some embodiments, L2 is attached to a side of chain of a subunit of AA.

In some embodiments, a Drug Linker ˜-L1-AA-L2-D has the following general formula:

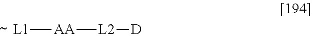

or a salt thereof, wherein L1 is a Stretcher unit; AA is an Amino Acid unit having from one to 12 subunits selected from alpha, beta and gamma amino acids and derivatives thereof, Sugar units, Carboxyl units and amino acid subunits optionally substituted with at least one PEG unit; L2 is a Linker Subunit optionally substituted with at least one Sugar unit, PEG unit, Carboxyl unit or a combination thereof; D is a Drug unit; and the wavy line (˜) indicates an attachment site for a Targeting unit; provided that ˜AA-L2-comprises at least one Sugar unit, PEG unit, Carboxyl unit or a combination thereof. In some embodiments, L2 is attached to a side of chain of a subunit of AA.

In some embodiments, a Linker intermediate ˜L1-AA≈ has the following general formula:

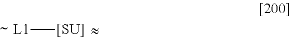

or a salt thereof, wherein [SU] is an Amino Acid unit, in which each SU is a Sugar unit, L1 is a Stretcher unit, the wavy line (˜) indicates an attachment site for a Targeting unit and the double wavy (≈) line indicates an attachment site for Linker Subunit L2.

An exemplary embodiment of such a Linker intermediate includes the following:

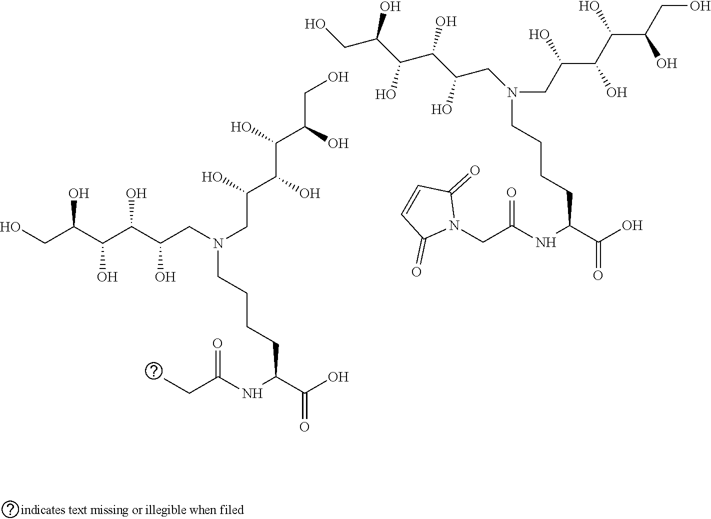

or a salt thereof, wherein the carboxyl group on the right side of the Sugar unit is the attachment site for Linker Subunit L2.

In some embodiments, a Linker intermediate ˜L1-AA≈ has the following general formula:

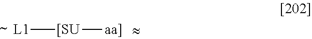

or a salt thereof, wherein [SU-aa-] is an Amino Acid unit, in which each SU is a Sugar unit and aa is an optional subunit of AA selected from alpha, beta and gamma amino acids and derivatives thereof, L1 is a Stretcher unit, the wavy line ( [symbol] ) indicates an attachment site for a Targeting unit; and the double wavy ( [symbol] ) line indicates an attachment site for Linker Subunit L2. In some embodiments, aa is an amino acid selected from glycine, lysine and glutamate. In some embodiments, [SU-aa] is [SU-Lys-].

An exemplary embodiment of such a Linker intermediate includes the following:

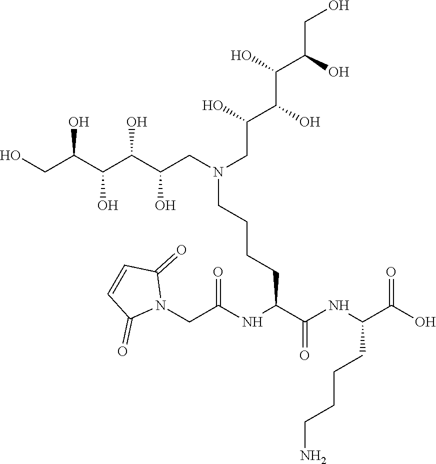

or a salt thereof, wherein the carboxyl group on the right side of the lysine is the attachment site for Linker Subunit L2.

In some embodiments, a Linker intermediate ˜L1-AA≈ has the following general formula:

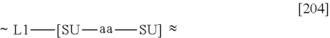

or a salt thereof, wherein [SU-aa-SU] is an Amino Acid unit, in which each SU is a Sugar unit and aa is a subunit of AA selected from alpha, beta and gamma amino acids and derivatives thereof, L1 is a Stretcher unit, the wavy line ( [symbol] ) indicates an attachment site for a Targeting unit and the double wavy ( [symbol] ) line indicates an attachment site for Linker Subunit L2. In some embodiments, aa is an amino acid selected from glycine, lysine and glutamate. In some embodiments, [SU-aa-SU] is [SU-Lys-SU].

An exemplary embodiment of such a Linker intermediate includes the following:

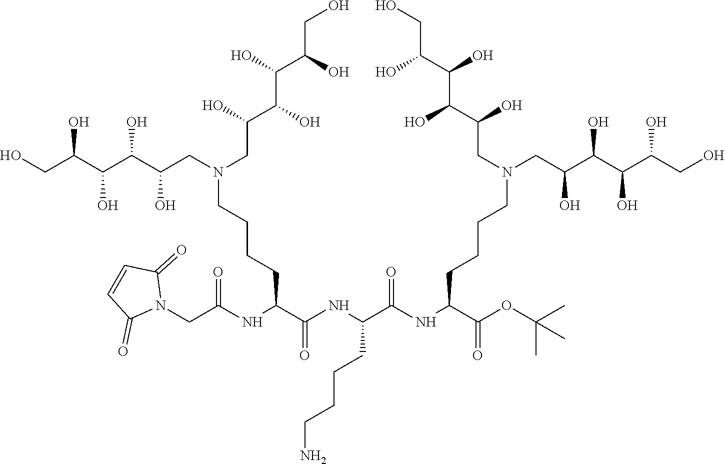

or a salt thereof, wherein the protected carboxyl group on the right side of the Sugar unit is the attachment site for Linker Subunit L2.

In some embodiments, a Linker intermediate ˜AA-L2≈ has the following general formula:

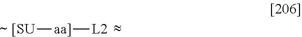

or a salt thereof, wherein [SU-aa] is an Amino Acid unit, in which each SU is a Sugar unit and aa is an optional subunit of AA selected from alpha, beta and gamma amino acids and derivatives thereof, L2 is a Linker Subunit optionally substituted with at least one Sugar unit, PEG unit, Carboxyl unit or a combination thereof, the wavy line (˜) indicates an attachment site for a Stretcher unit, and the double wavy (≈) line indicates an attachment site for a Drug unit. In some embodiments, aa is an amino acid selected from glycine, lysine, and glutamate. In some embodiments, aa is lysine. In some embodiments, [Su-aa] is [Su-Lys]. In some embodiments, Linker Subunit L2 is a cleavable linker subunit.

An exemplary embodiment of such a Linker includes the following:

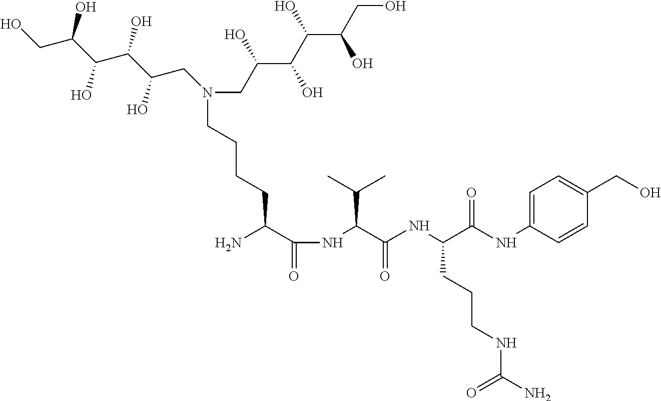

or a salt thereof, wherein the amino group on the left side of the Sugar unit is the attachment site for the Stretcher unit and the benzylic alcohol group on the right side is the attachment site for the Drug unit.

In some embodiments, a Linker intermediate ˜AA-L2≈ has the following general formula:

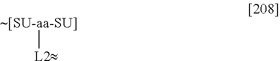

or a salt thereof, wherein [SU-aa-SU] is an Amino Acid unit, in which each SU is a Sugar unit and aa is a subunit of AA selected from alpha, beta and gamma amino acids and derivatives thereof, L2 is a Linker Subunit optionally substituted with at least one Sugar unit, PEG unit, Carboxyl unit or a combination thereof, and L2 is attached to a site on aa, the wavy line ( [symbol] ) indicates an attachment site for a Stretcher unit, and the double wavy ( [symbol] ) line indicates an attachment site for a Drug unit. In some embodiments, aa is an amino acid selected from glycine, lysine and glutamate. In some embodiments, aa is lysine. In some embodiments, [Su-aa-Su] is [Su-Lys-Su]. In some embodiments, Linker Subunit L2 is a cleavable linker subunit.

Exemplary embodiments of such a Linker intermediate include the following:

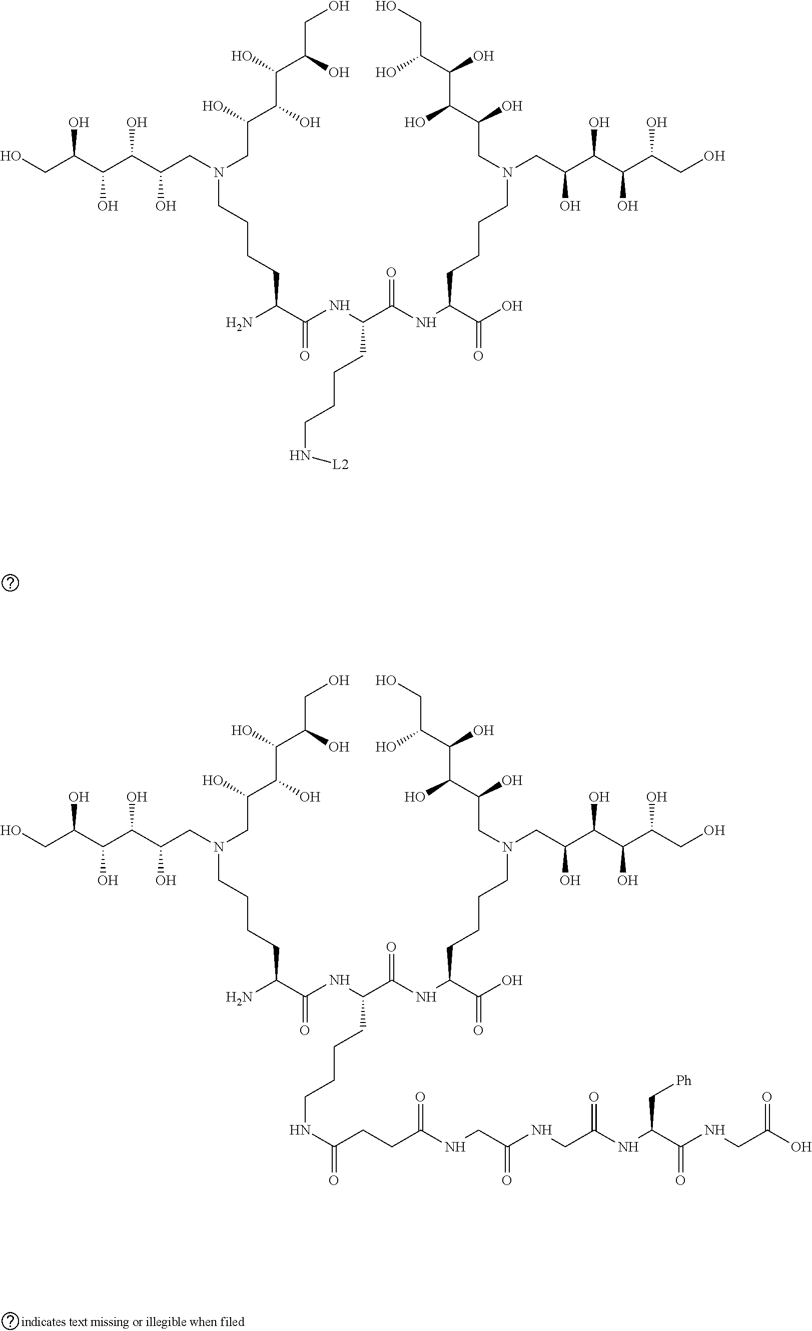

or a salt thereof, wherein the amino group on the left side of the Sugar units is the attachment site for the Stretcher unit, and the Drug unit is attached to the terminal acid group or the benzyl alcohol (i.e., the H is removed from the benzyilic alcohol and a bond formed between the benzylic oxygen and the Drug unit).

In some embodiments, a Linker has the following general formula:

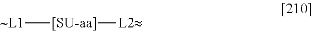

or a salt thereof, wherein [SU-aa-SU] is an Amino Acid unit, in which each SU is a Sugar unit and aa is an optional subunit of AA selected from alpha, beta and gamma amino acids and derivatives thereof, L1 is a Stretcher unit, L2 is a Linker Subunit optionally substituted with at least one Sugar unit, PEG unit, Carboxyl unit or a combination thereof, and L2 is attached to a site on aa or to SU, the wavy line (˜) indicates an attachment site for a Targeting unit, and the double wavy (≈) line indicates an attachment site for a Drug unit. In some embodiments, aa is an amino acid selected from glycine, lysine and glutamate. In some embodiments, aa is present and is lysine.

Exemplary embodiments of such a Linker include the following:

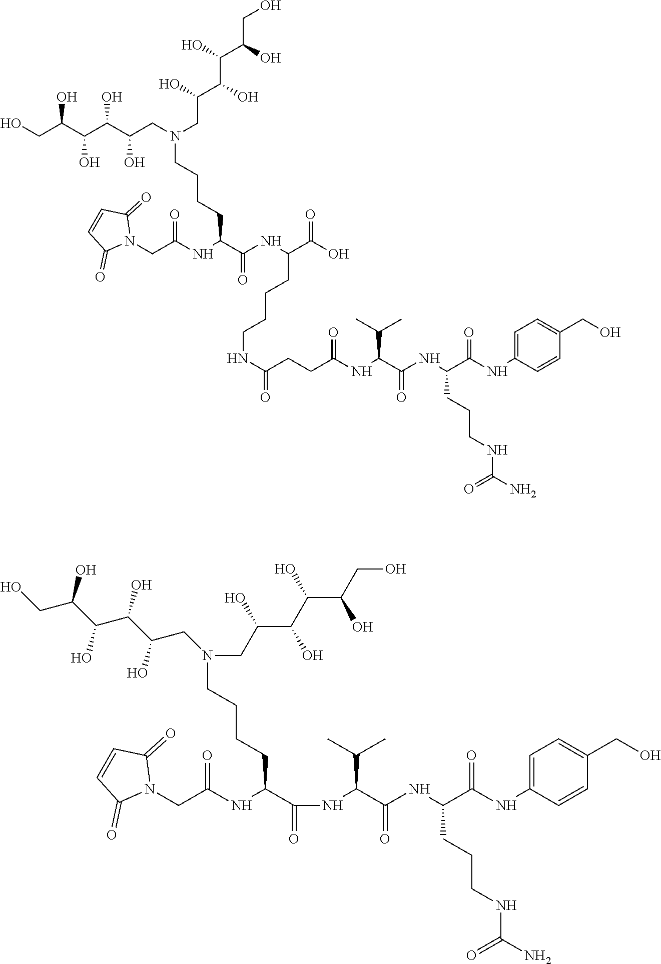

or a salt thereof, wherein the maleimide group on the left side of the Sugar units is the attachment site for the Targeting unit, and the Drug unit is attached to the benzyl alcohol (i.e., the H is removed from the benzyilic alcohol and a bond formed between the benzylic oxygen and the Drug unit).

In some embodiments, a Linker has the following general formula:

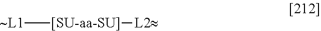

or a salt thereof, wherein [SU-aa-SU] is an Amino Acid unit, in which each SU is a Sugar unit and aa is an optional subunit of AA selected from alpha, beta and gamma amino acids and derivatives thereof, L1 is a Stretcher unit, L2 is a Linker Subunit optionally substituted with at least one Sugar unit, PEG unit, Carboxyl unit or a combination thereof, and L2 is attached to AA, the wavy line (˜) indicates an attachment site for a Targeting unit and the double wavy (=) line indicates an attachment site for a Drug unit. In some embodiments, aa is an amino acid selected from glycine, lysine and glutamate. In some embodiments, aa is present and is lysine.

Exemplary embodiments of such a Linker include the following:

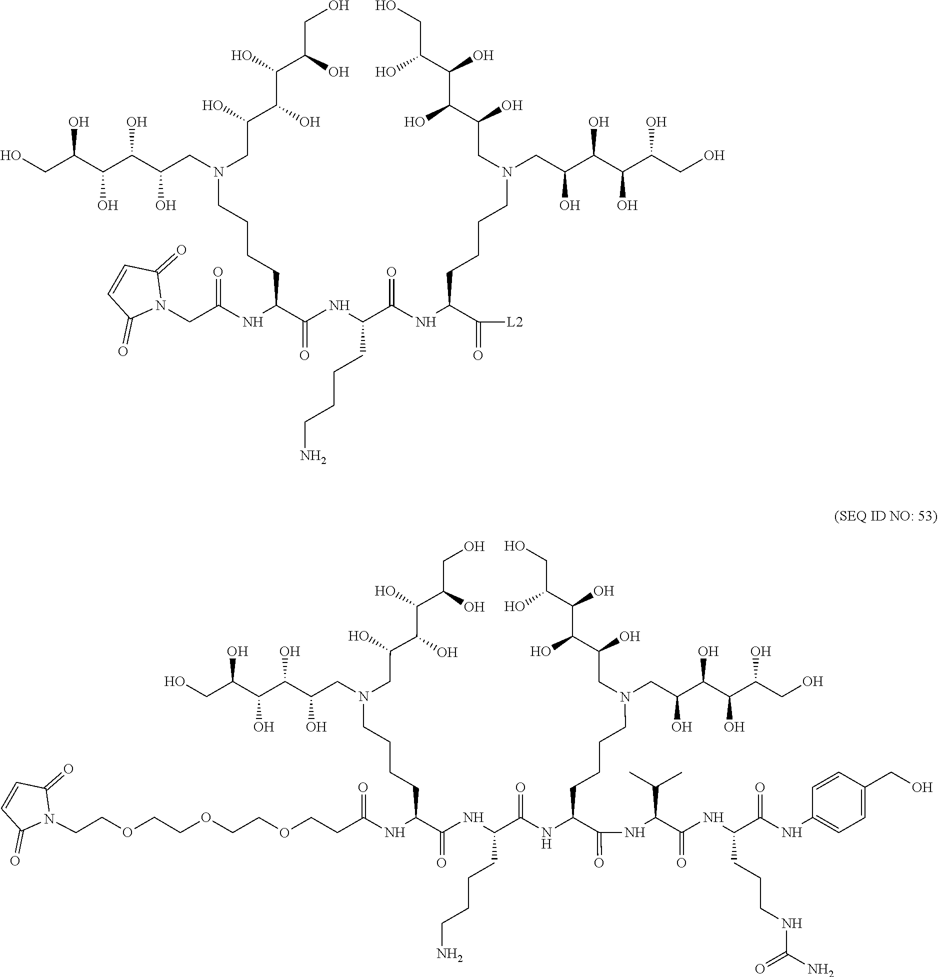

or a salt thereof, wherein the maleimide group on the left side of the Sugar units is the attachment site for the Targeting unit, and the Drug unit is attached to the benzyl alcohol (i.e., the H is removed from the benzyilic alcohol and a bond formed between the benzylic oxygen and the Drug unit).

In some embodiments, a Linker has the following general formula:

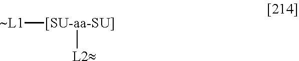

or a salt thereof, wherein [SU-aa-SU] is an Amino Acid unit, in which each SU is a Sugar unit and aa is a subunit of AA selected from alpha, beta and gamma amino acids and derivatives thereof, L1 is a Stretcher unit, L2 is a Linker Subunit optionally substituted with at least one Sugar unit, PEG unit, Carboxyl unit or a combination thereof, L2 is attached to a site on aa, the wavy line (˜) indicates an attachment site for a Targeting unit, and the double wavy (≈) line indicates an attachment site for a Drug unit. In some embodiments, aa is an amino acid selected from glycine, lysine and glutamate. In some embodiments, aa is present and is lysine.

Exemplary embodiments of such a Linker include the following:

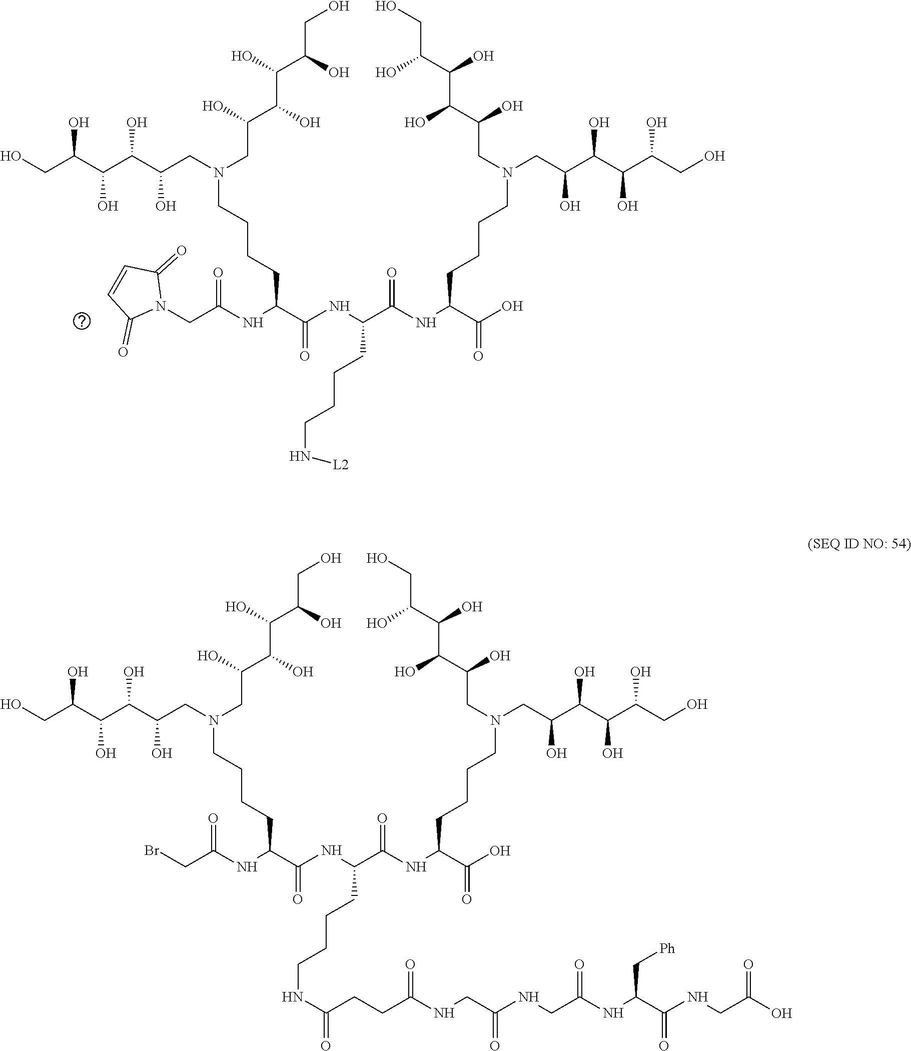

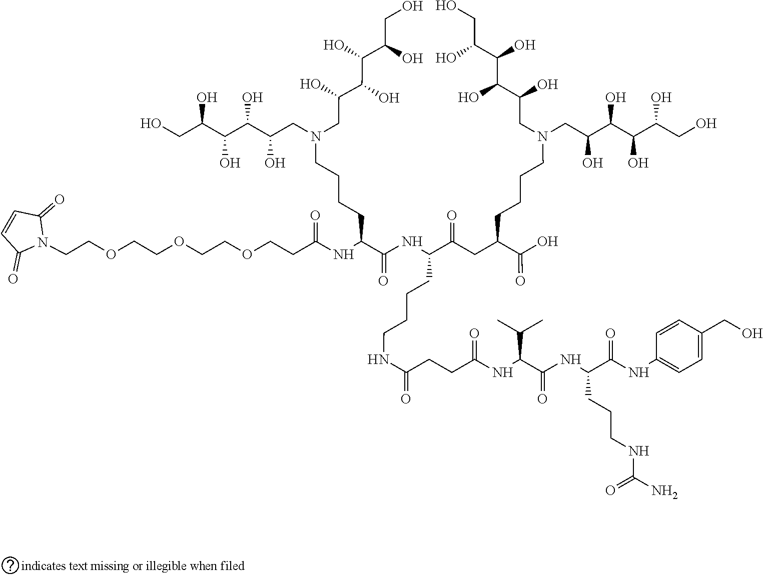

or a salt thereof, wherein the maleimide or bromoacetamide group on the left side of the Sugar units is the attachment site for the Targeting unit, and the Drug unit is attached to the terminal acid group or the benzyl alcohol (i.e., the H is removed from the benzyilic alcohol and a bond formed between the benzylic oxygen and the Drug unit).

In some embodiments, a Drug-Linker intermediate ˜AA-L2-D has the following general formula:

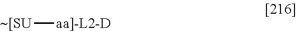

or a salt thereof, wherein [SU-aa] is an Amino Acid unit, in which each SU is a Sugar unit and aa is an optional subunit of AA selected from alpha, beta and gamma amino acids and derivatives thereof, L2 is a Linker Subunit optionally substituted with at least one Sugar unit, PEG unit, Carboxyl unit or a combination thereof, D is a Drug unit, and the wavy line (˜) indicates an attachment site for a Stretcher unit. In some embodiments, aa is an amino acid selected from glycine, lysine and glutamate. In some embodiments, aa is lysine. In some embodiments, [Su-aa] is [Su-Lys]. In some embodiments, Linker Subunit L2 is a cleavable linker subunit.

An exemplary embodiment of such a Drug-Linker includes the following:

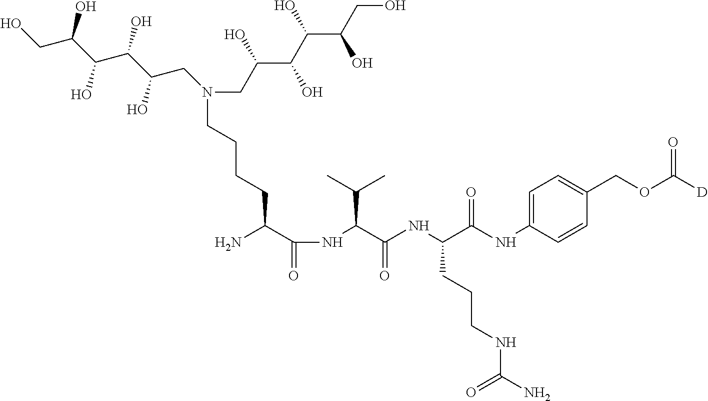

or a salt thereof, wherein the amino group on the left side of the Sugar unit is the attachment site for the Stretcher unit.

In some embodiments, a Drug-Linker intermediate ˜AA-L2-D has the following general formula:

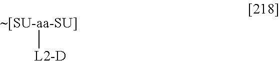

or a salt thereof, wherein [SU-aa-SU] is an Amino Acid unit, in which each SU is a Sugar unit and aa is a subunit of AA selected from alpha, beta and gamma amino acids and derivatives thereof, L2 is a Linker Subunit optionally substituted with at least one Sugar unit, PEG unit, Carboxyl unit or a combination thereof, and L2 is attached to a site on aa, D is a Drug unit, and the wavy line (˜) indicates an attachment site for a Stretcher unit. In some embodiments, aa is an amino acid selected from glycine, lysine and glutamate. In some embodiments, aa is lysine. In some embodiments, [Su-aa-Su] is [Su-Lys-Su]. In some embodiments, Linker Subunit L2 is a cleavable linker subunit.

Exemplary embodiments of such a Drug-Linker intermediate include the following:

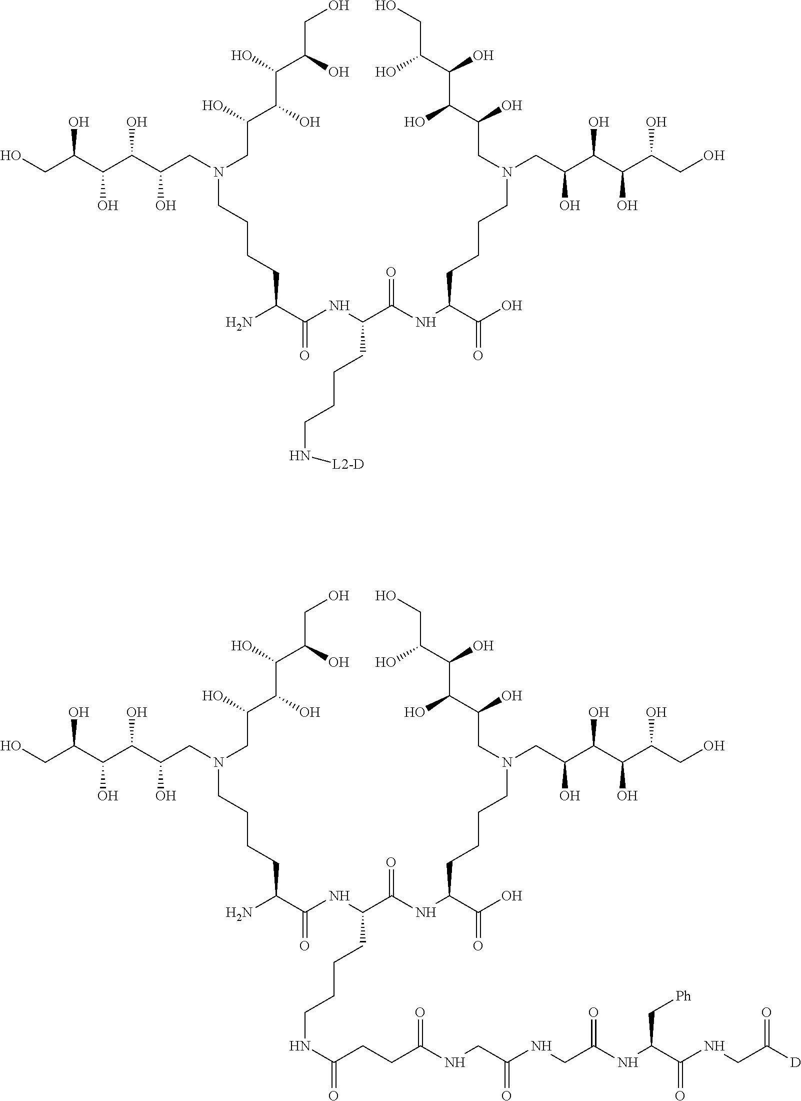

or a salt thereof, wherein the amino group on the left side of the Sugar unit is the attachment site for the Stretcher unit.

In some embodiments, a Linker intermediate ˜AA-L2≈ has the following general formula:

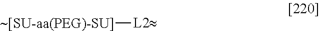

or a salt thereof, wherein [SU-aa(PEG)—SU] is an Amino Acid unit, in which each SU is a Sugar unit, aa is a subunit of AA selected from alpha, beta and gamma amino acids and derivatives thereof, PEG is a PEG unit attached to aa, L2 is a Linker Subunit optionally substituted with at least one Sugar unit, PEG unit, Carboxyl unit or a combination thereof, L2 is attached to AA, the wavy line (˜) indicates an attachment site for a Stretcher unit, and the double wavy (≈) line indicates an attachment site for a Drug unit. In some embodiments, aa is an amino acid selected from glycine, lysine and glutamate. In some embodiments, aa is lysine. In some embodiments, Linker Subunit L2 is a cleavable linker subunit.

An exemplary embodiment of such a Linker intermediate includes the following:

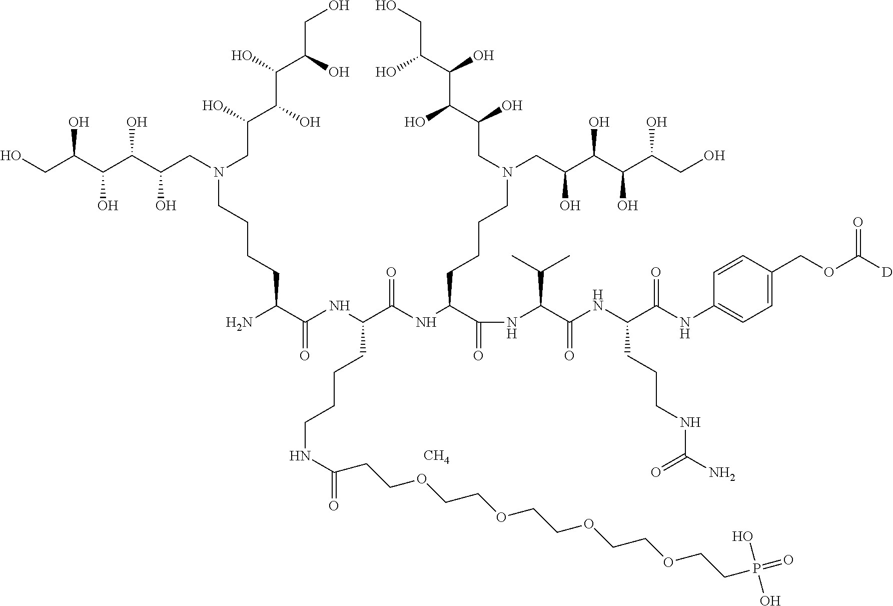

or a salt thereof, wherein the amino group on the left side of the Sugar unit is the attachment site for the Stretcher unit, and Drug unit is attached to the benzyl alcohol (i.e., the H is removed from the benzyilic alcohol and a bond formed between the benzylic oxygen and the Drug unit).

In some embodiments, a Drug-Linker intermediate ˜AA-L2-D has the following general formula:

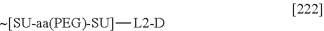

or a salt thereof, wherein [SU-aa(PEG)—SU] is an Amino Acid unit, in which each SU is a Sugar unit, aa is a subunit of AA selected from alpha, beta and gamma amino acids and derivatives thereof, PEG is a PEG unit attached to aa, L2 is a Linker Subunit optionally substituted with at least one Sugar unit, PEG unit, Carboxyl unit or a combination thereof, L2 is attached to AA, D is a Drug unit, and the wavy line (˜) indicates an attachment site for a Stretcher unit. In some embodiments, aa is an amino acid selected from glycine, lysine and glutamate. In some embodiments, aa is lysine. In some embodiments, Linker Subunit L2 is a cleavable linker subunit.

An exemplary embodiment of such a Drug-Linker intermediate includes the following:

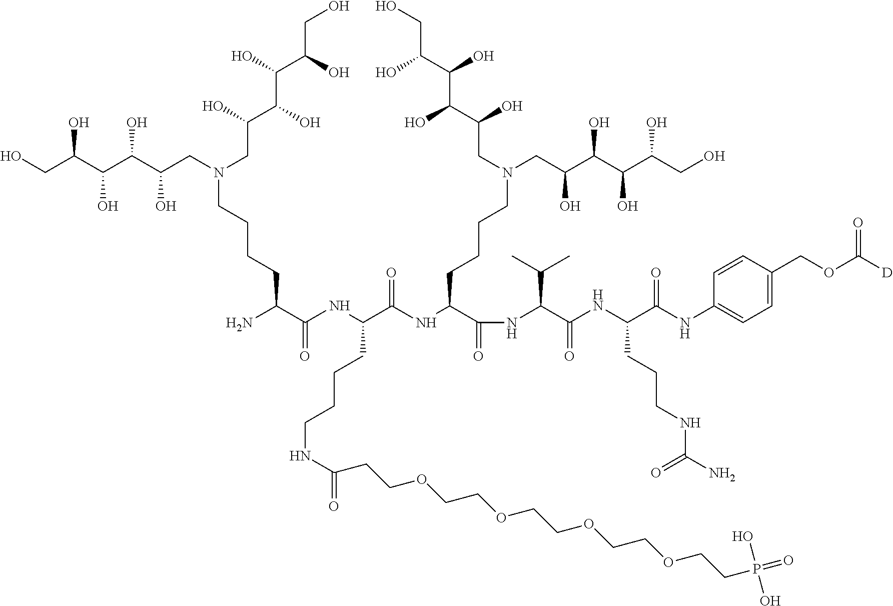

or a salt thereof, wherein the amino group on the left side of the Sugar unit is the attachment site for the Stretcher unit.

In some embodiments, a Linker intermediate ˜AA-L2≈ has the following general formula:

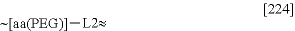

or a salt thereof, wherein [aa(PEG)] is an Amino Acid unit, in which aa is a subunit of AA selected from alpha, beta and gamma amino acids and derivatives thereof, PEG is a PEG unit attached to aa, L2 is a Linker Subunit optionally substituted with at least one Sugar unit, PEG unit, Carboxyl unit or a combination thereof, L2 is attached to AA, the wavy line (˜) indicates an attachment site for a Stretcher unit, and the double wavy (≈) line indicates an attachment site for a Drug unit. In some embodiments, aa is an amino acid selected from glycine, lysine and glutamate. In some embodiments, aa is lysine. In some embodiments, Linker Subunit L2 is a cleavable linker subunit.

Exemplary embodiments of such a Linker intermediate include the following:

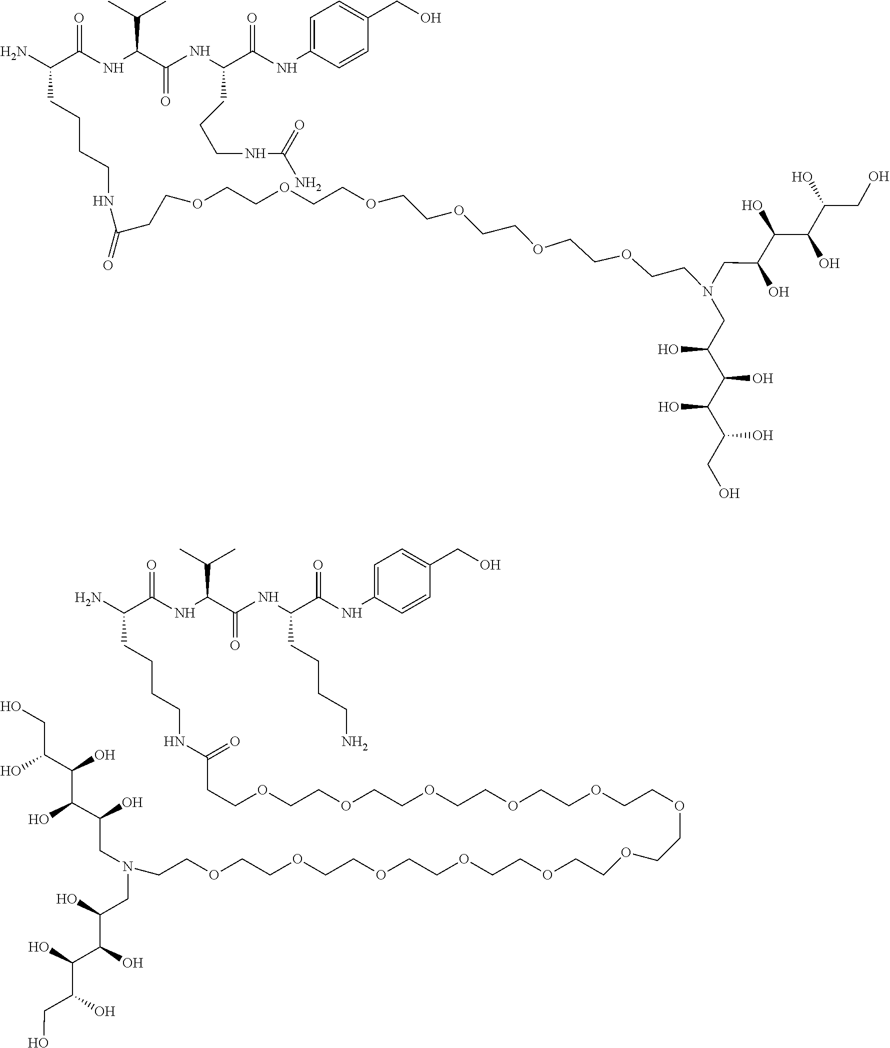

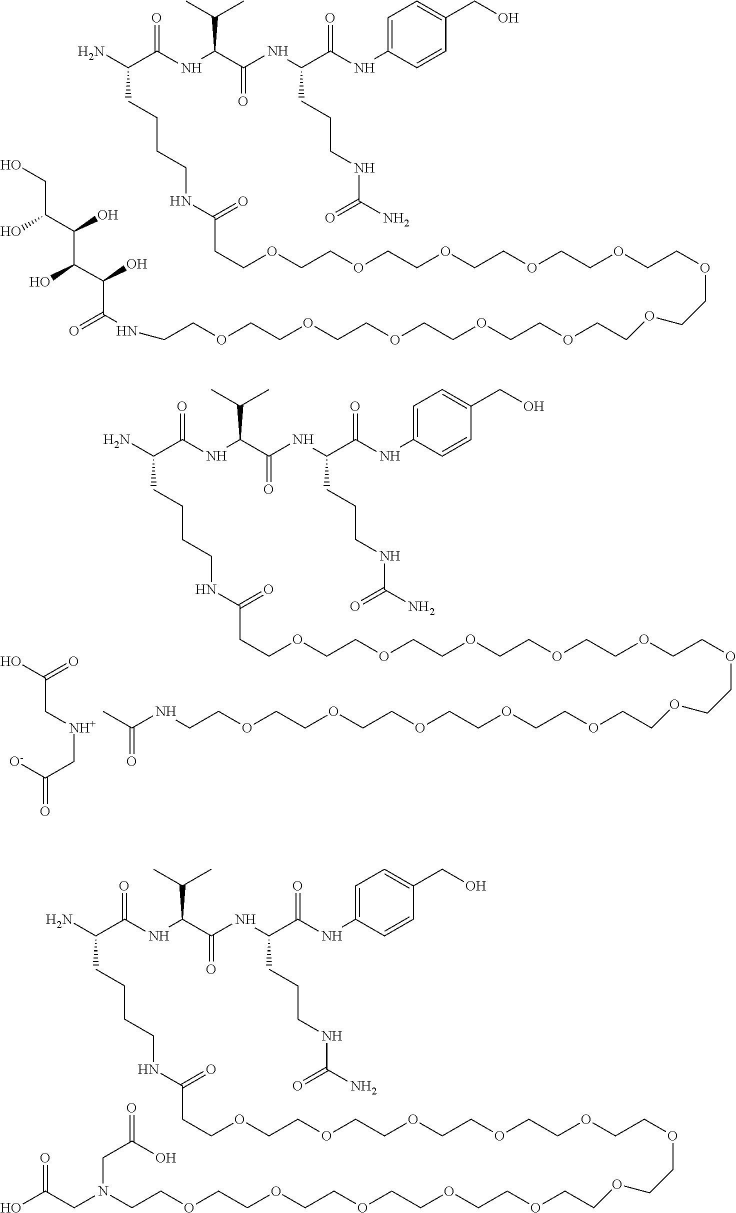

or a salt thereof, wherein the amino group on the left side of the molecule is the attachment site for the Stretcher unit.

In some embodiments, provided is a Linker intermediate or Linker, wherein ˜AA-L2˜ has one of the following structures:

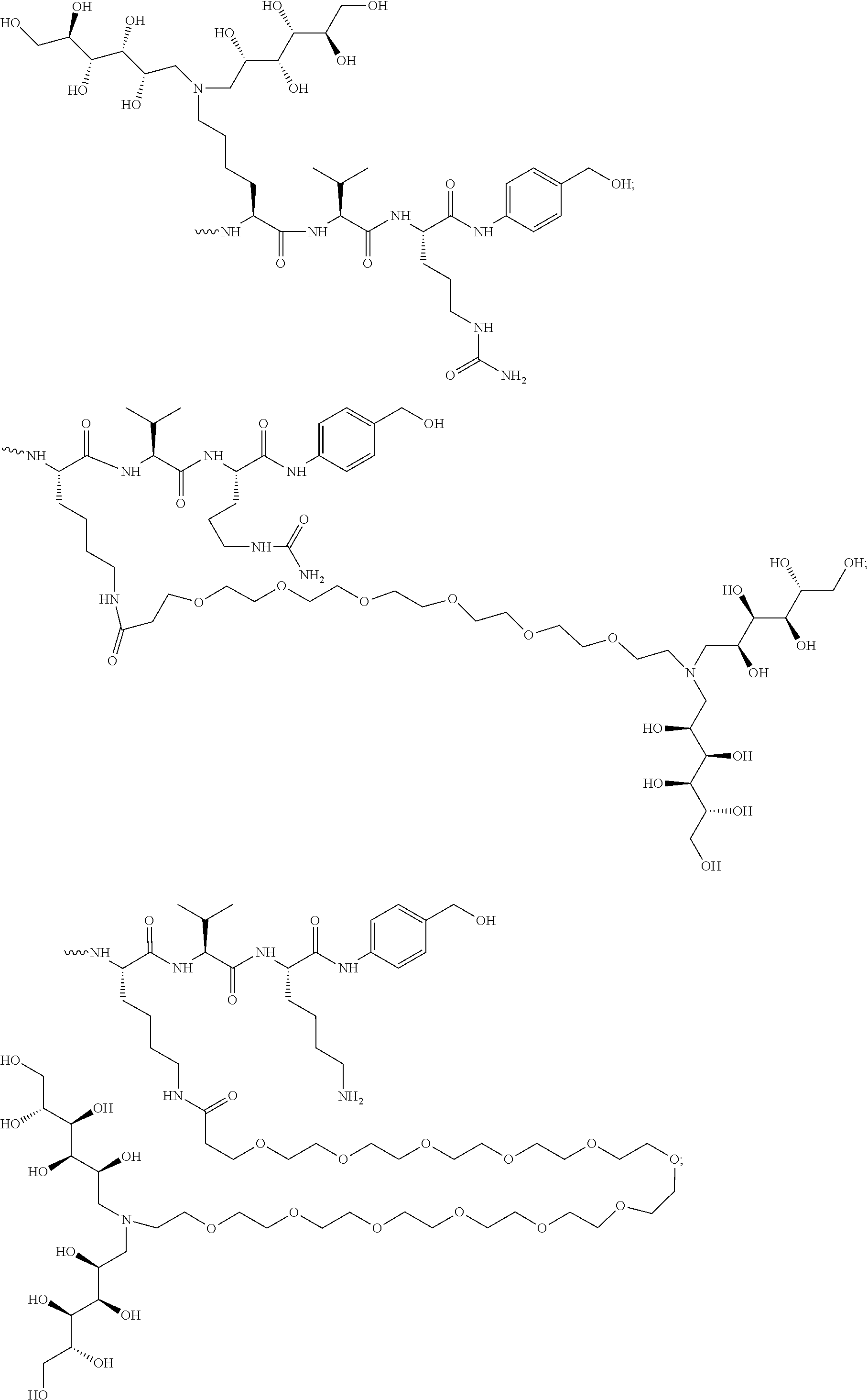

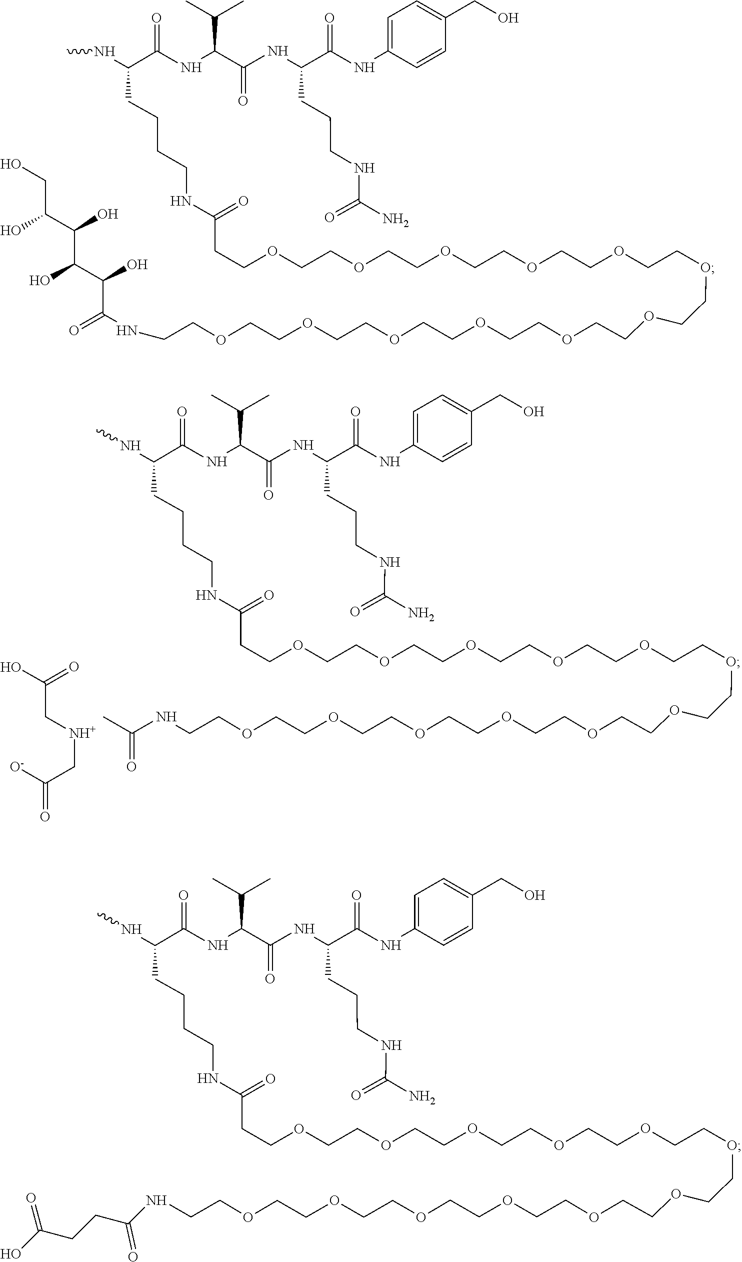

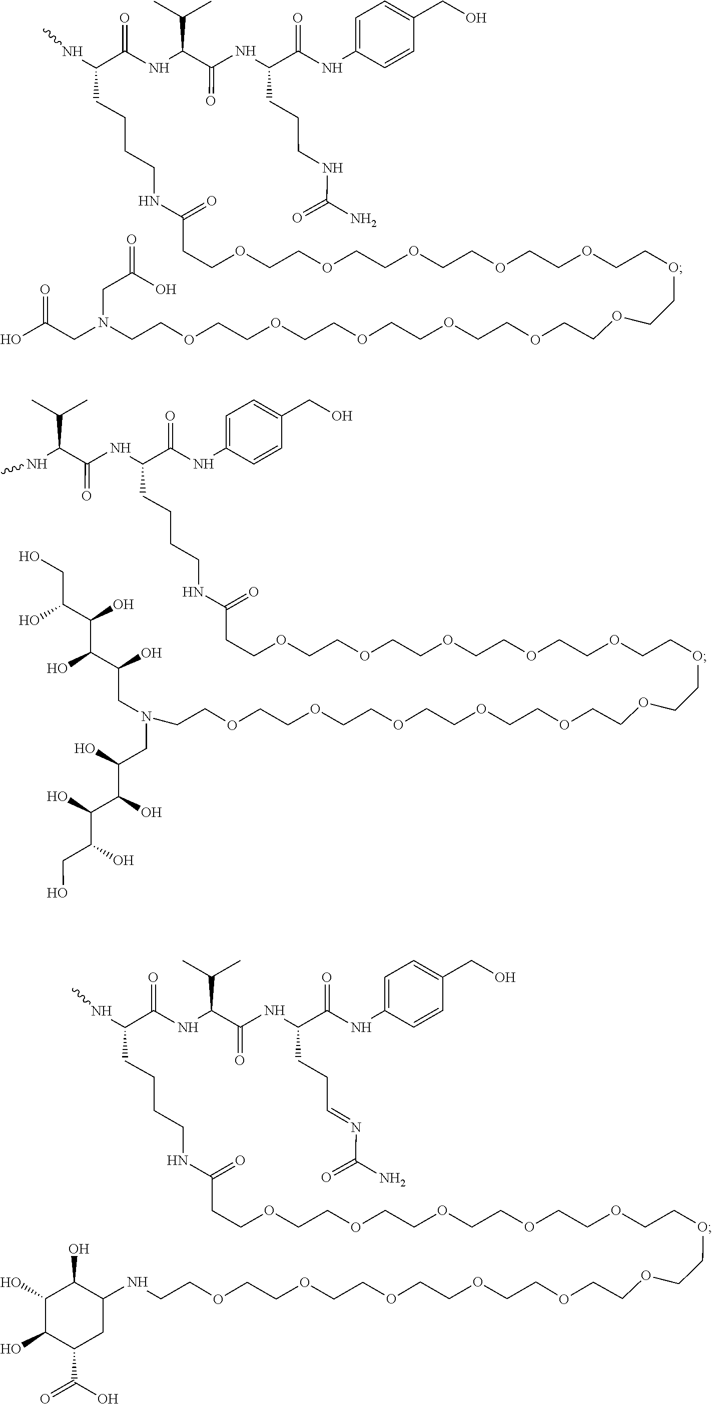

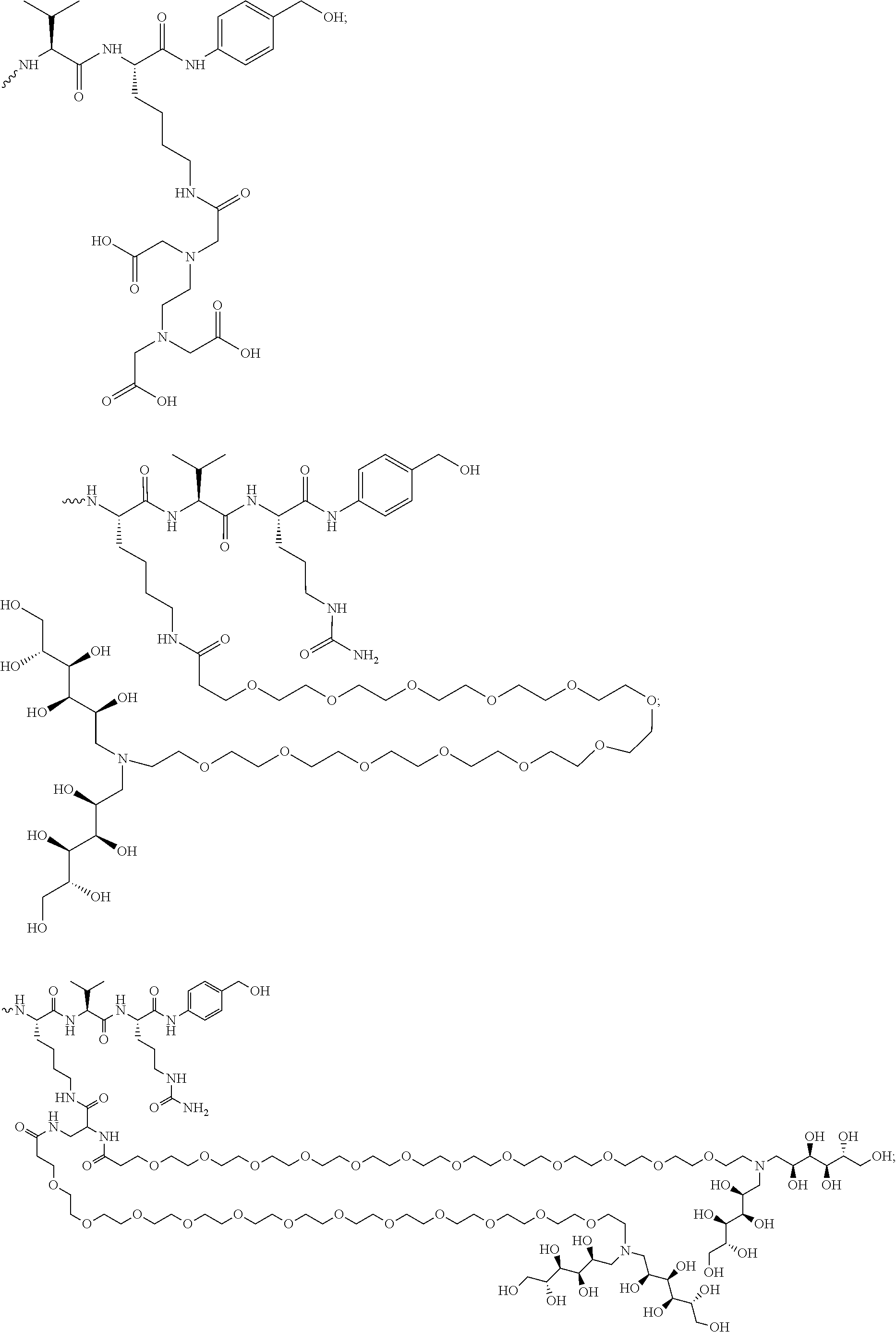

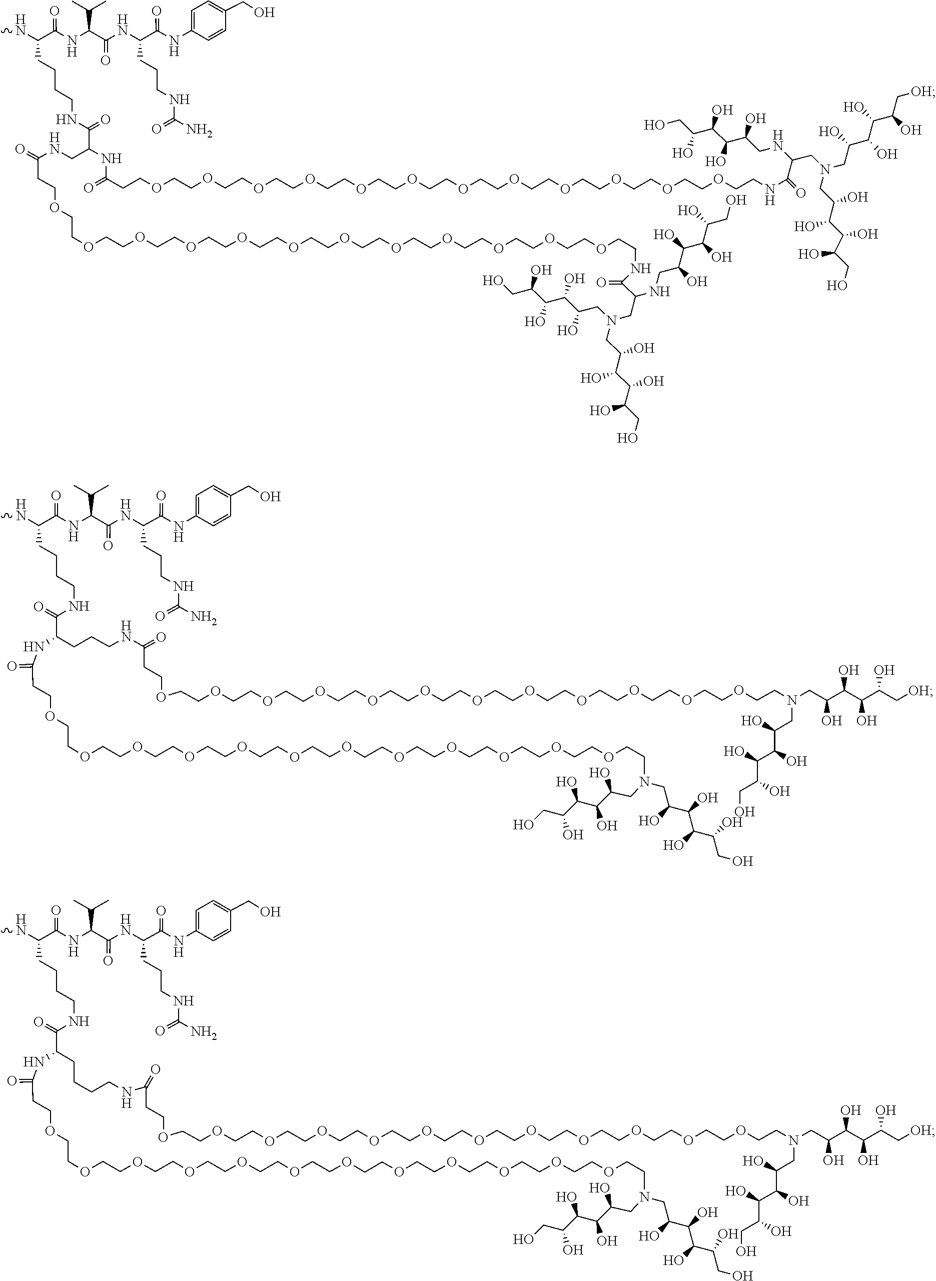

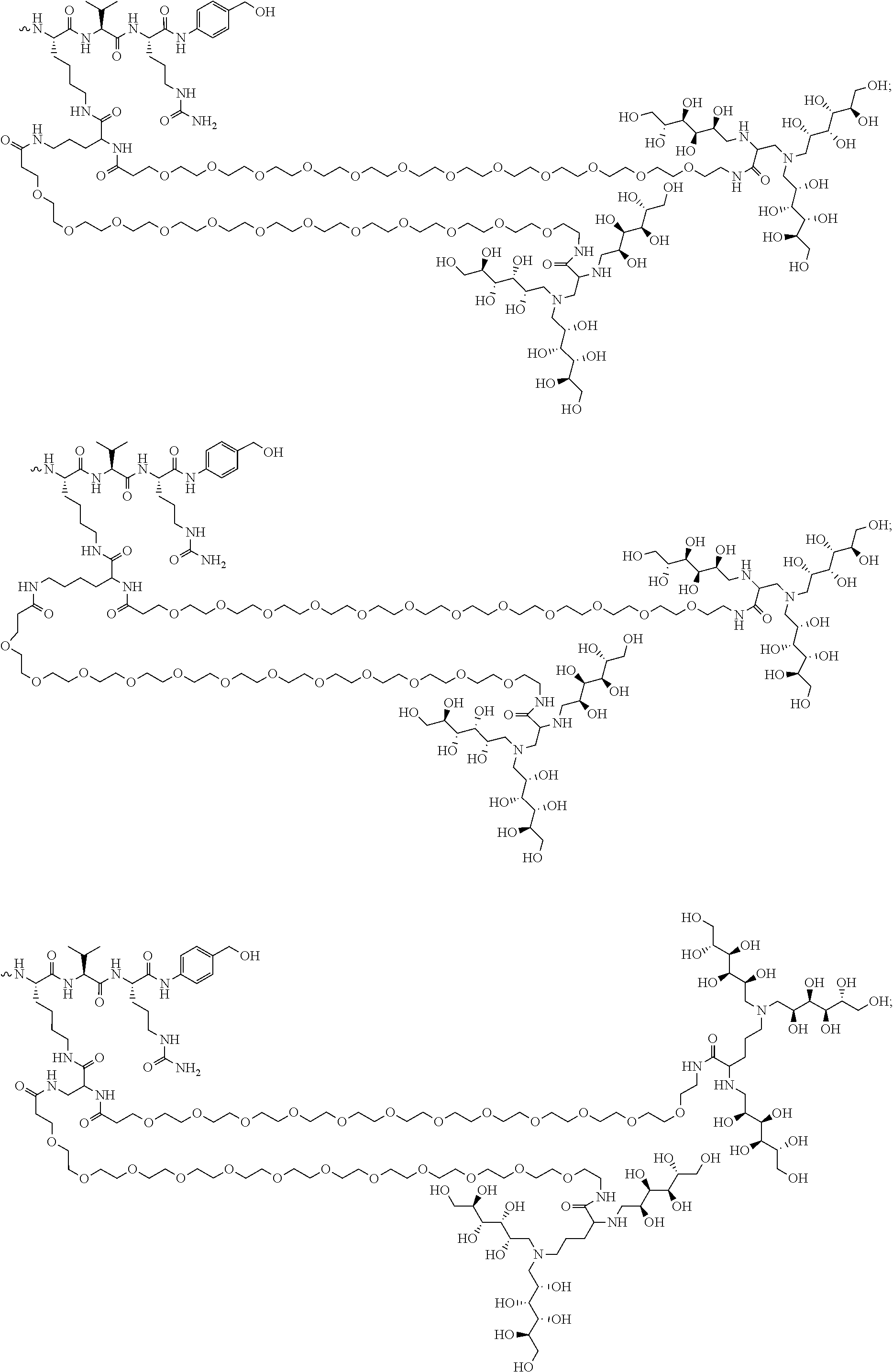

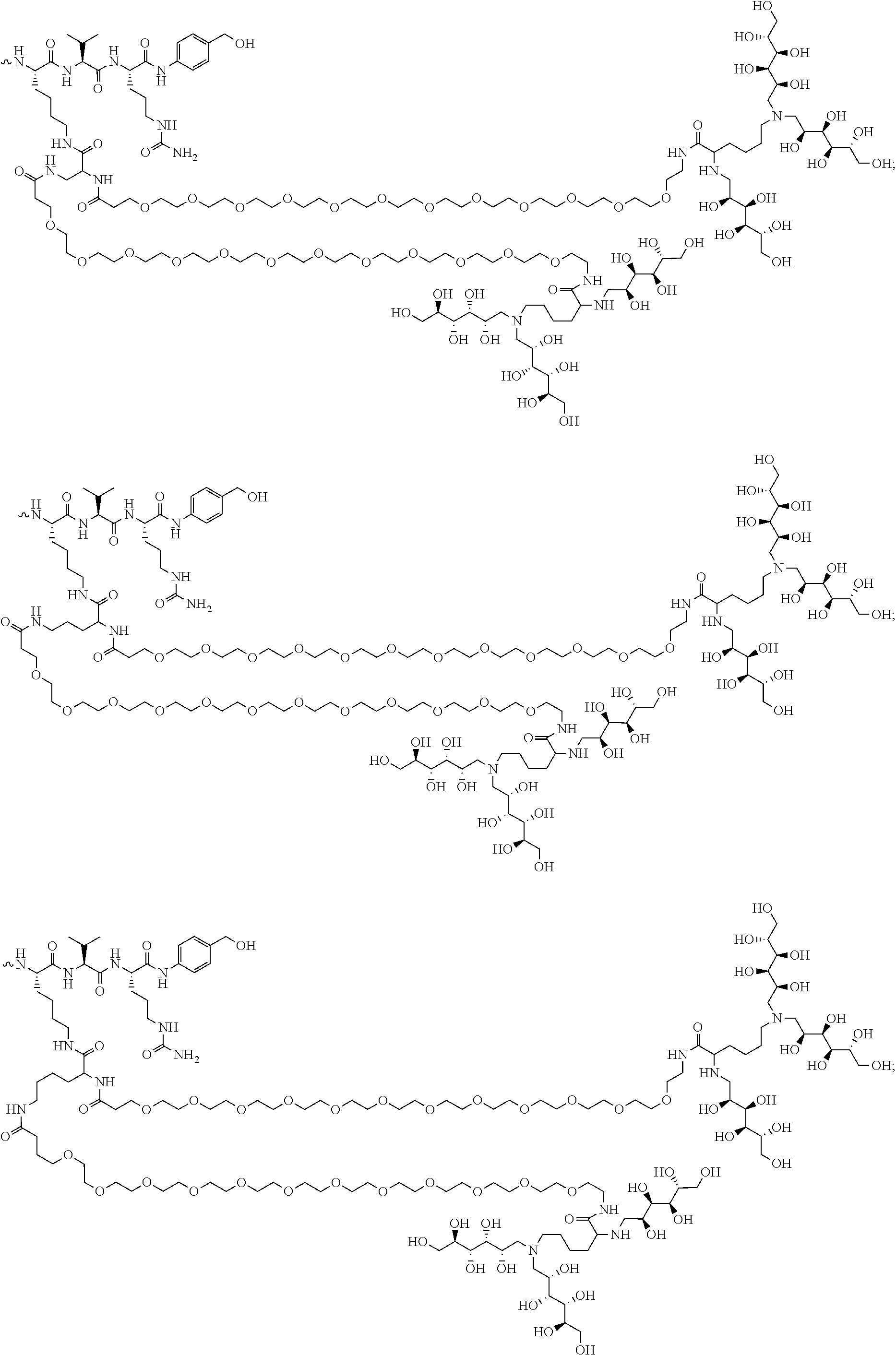

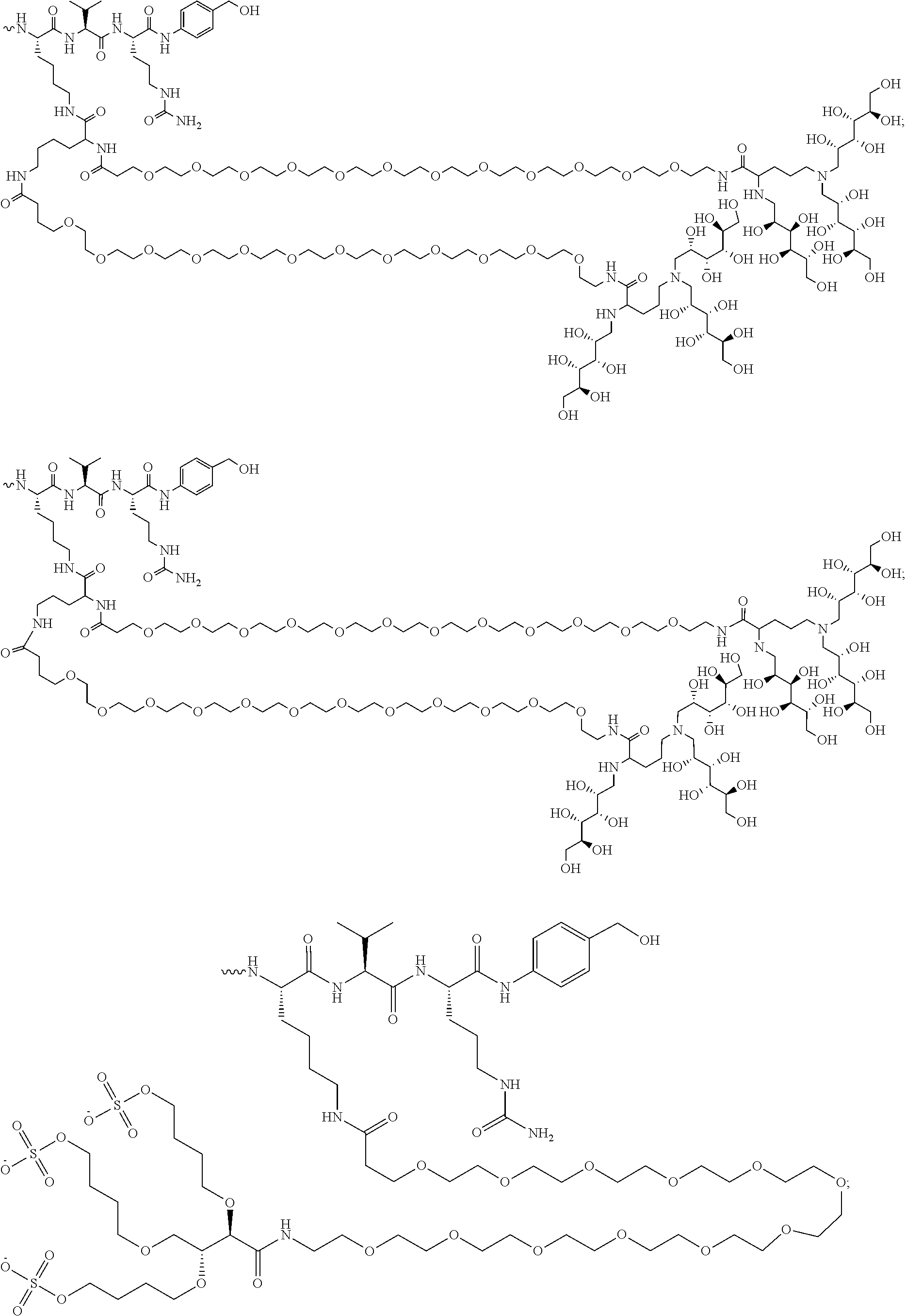

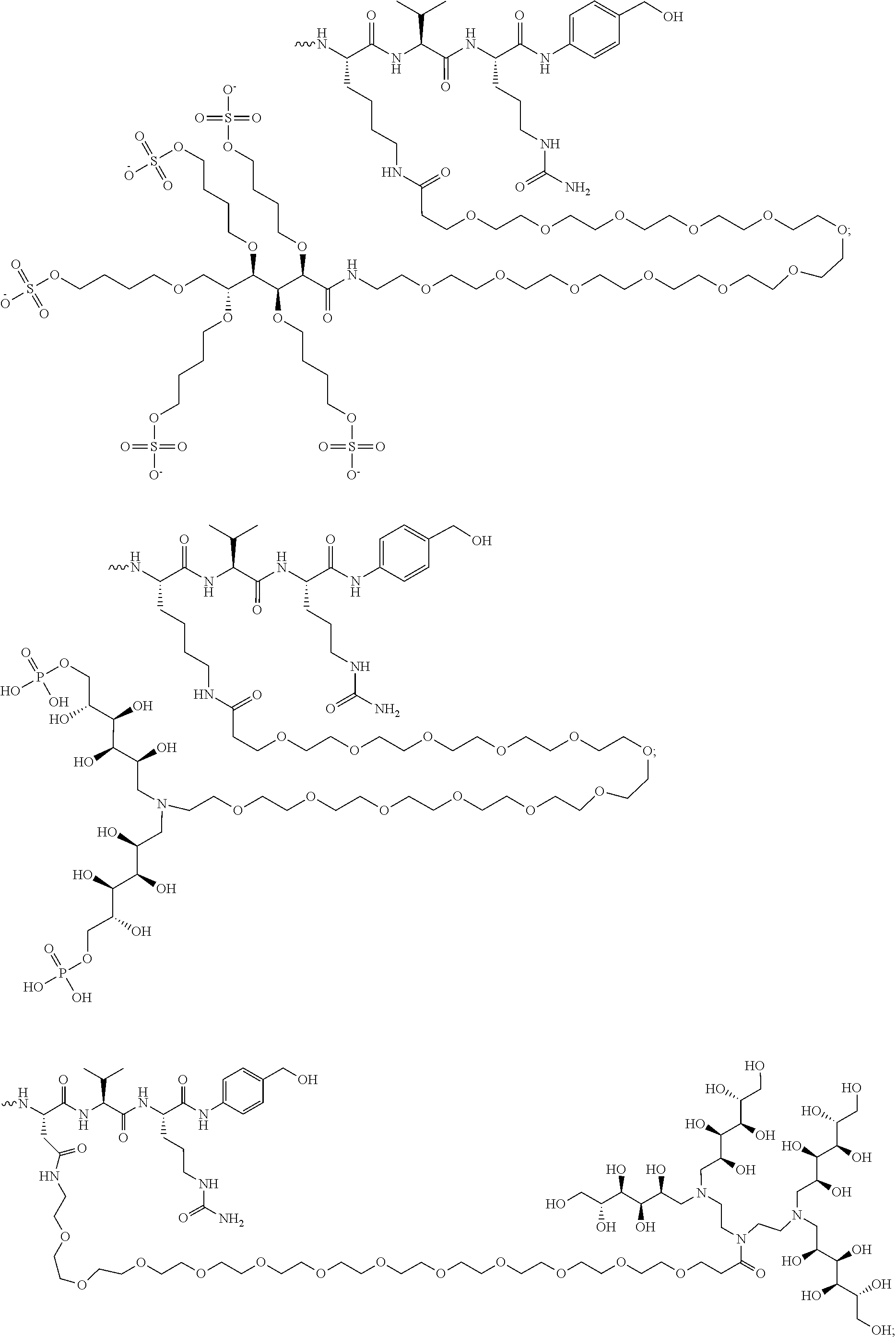

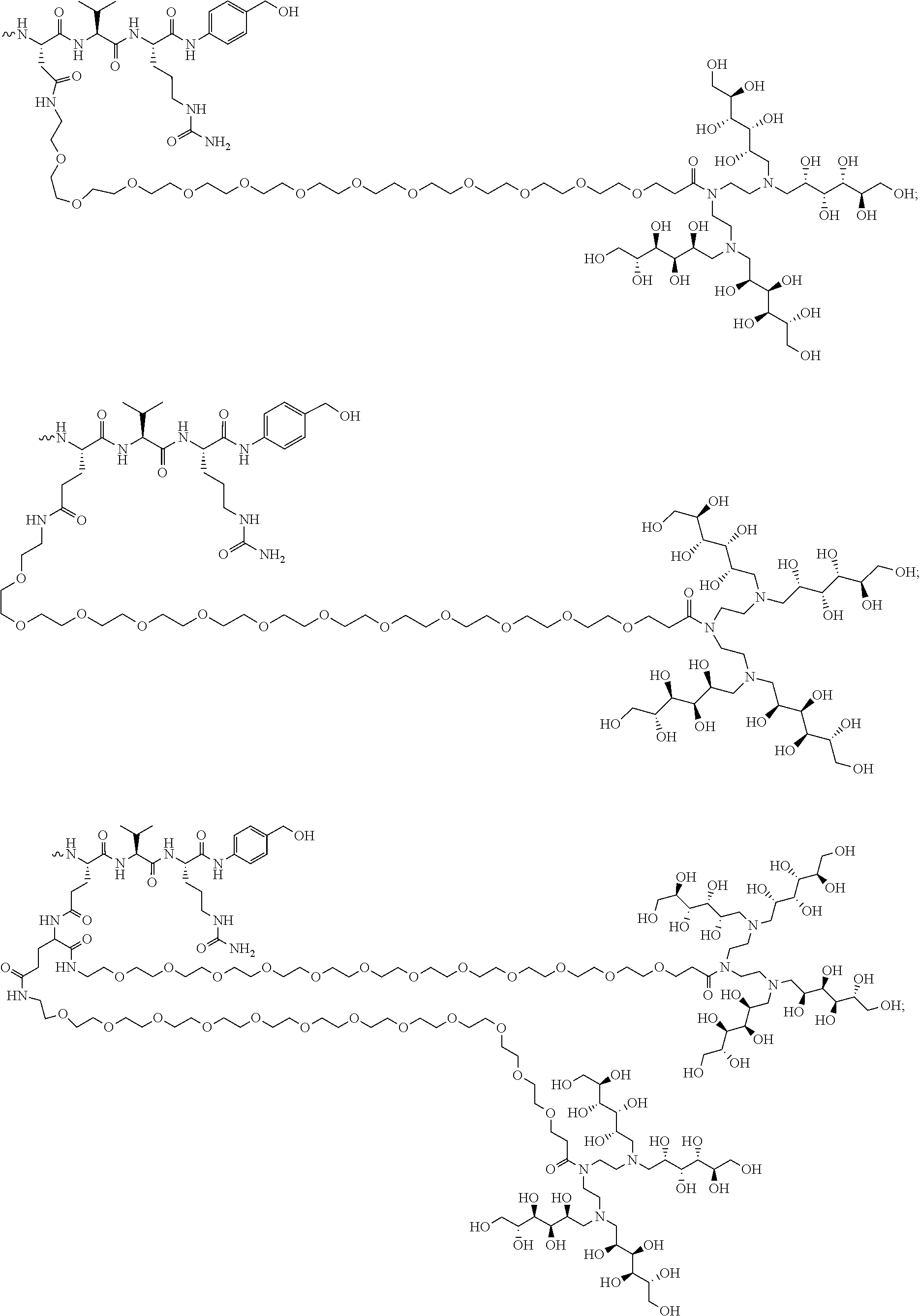

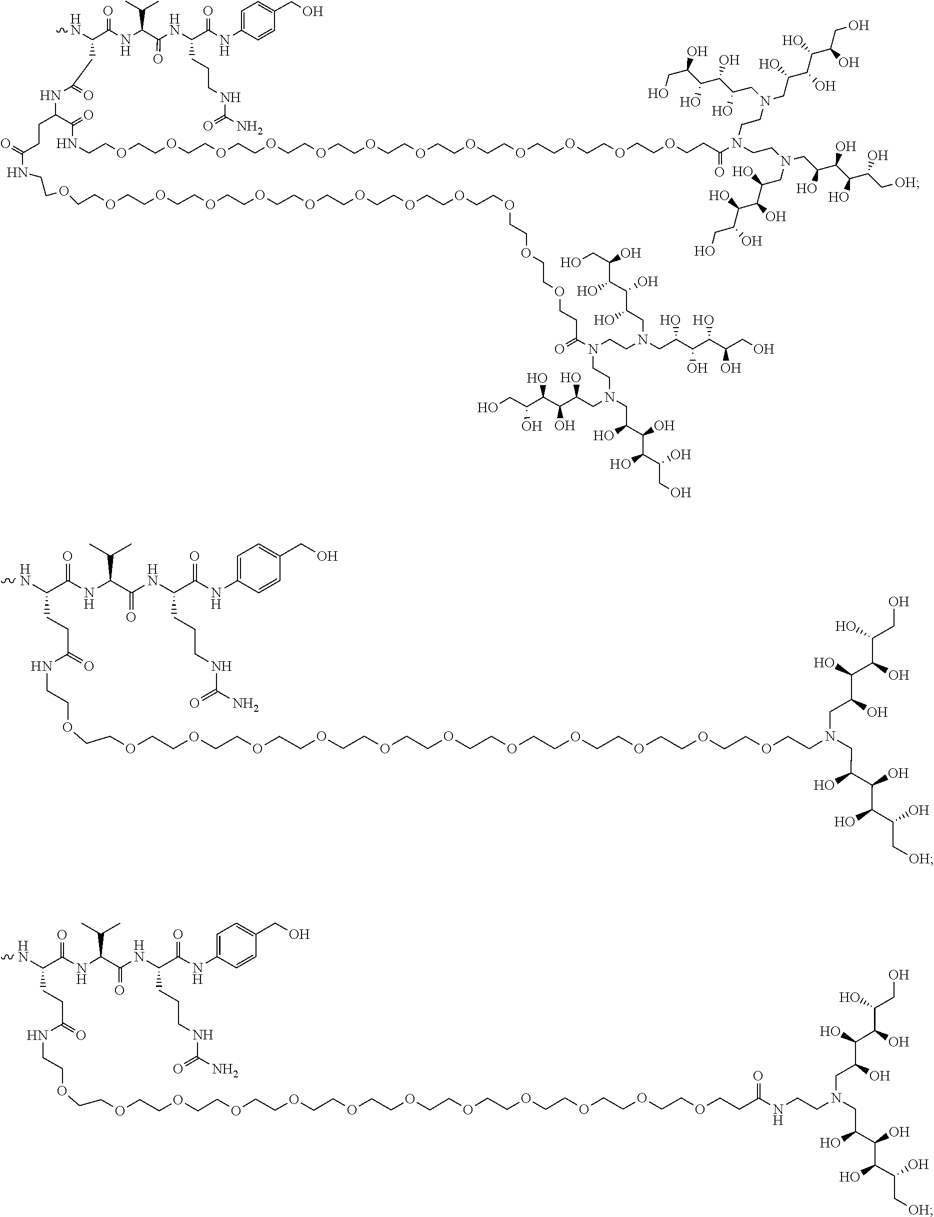

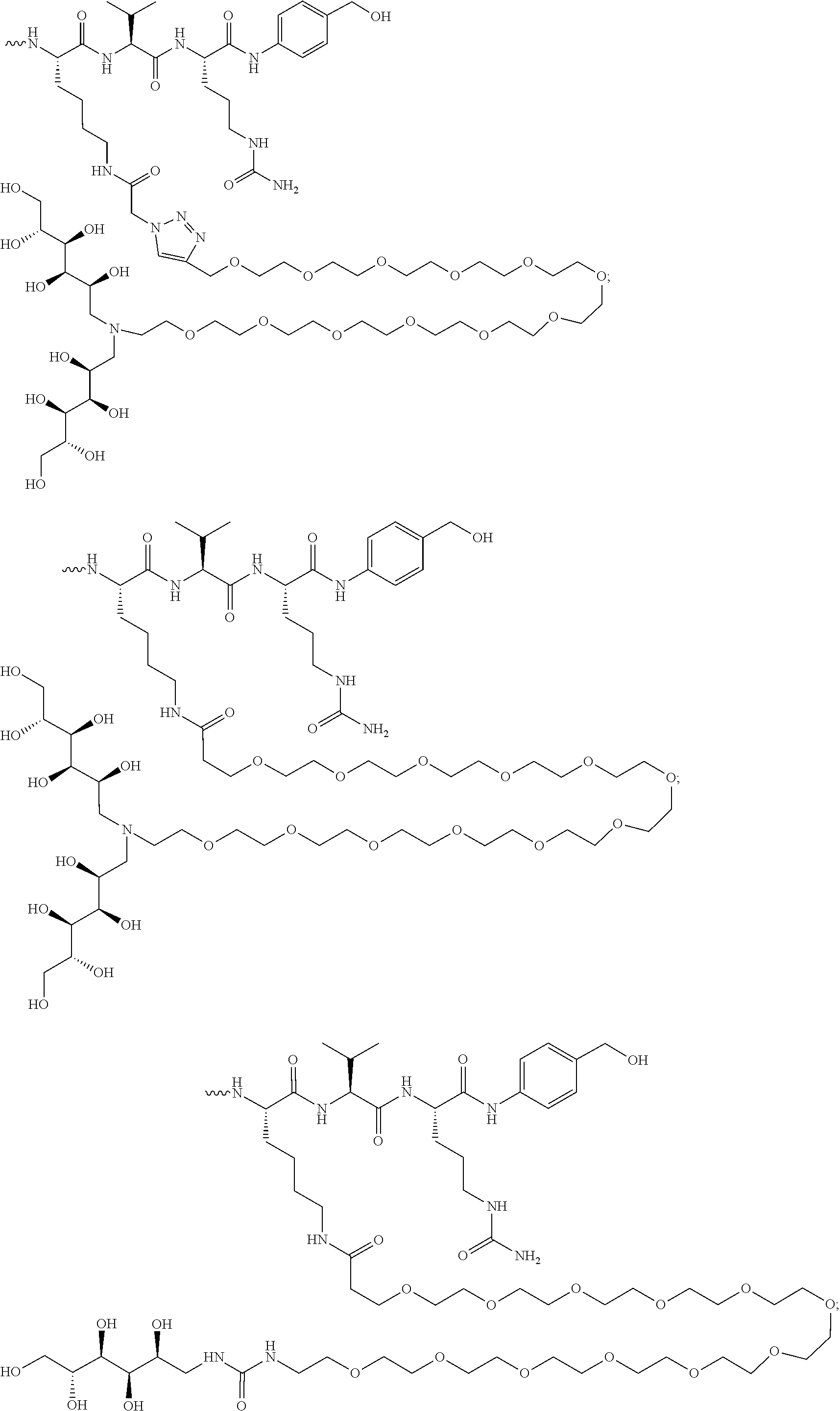

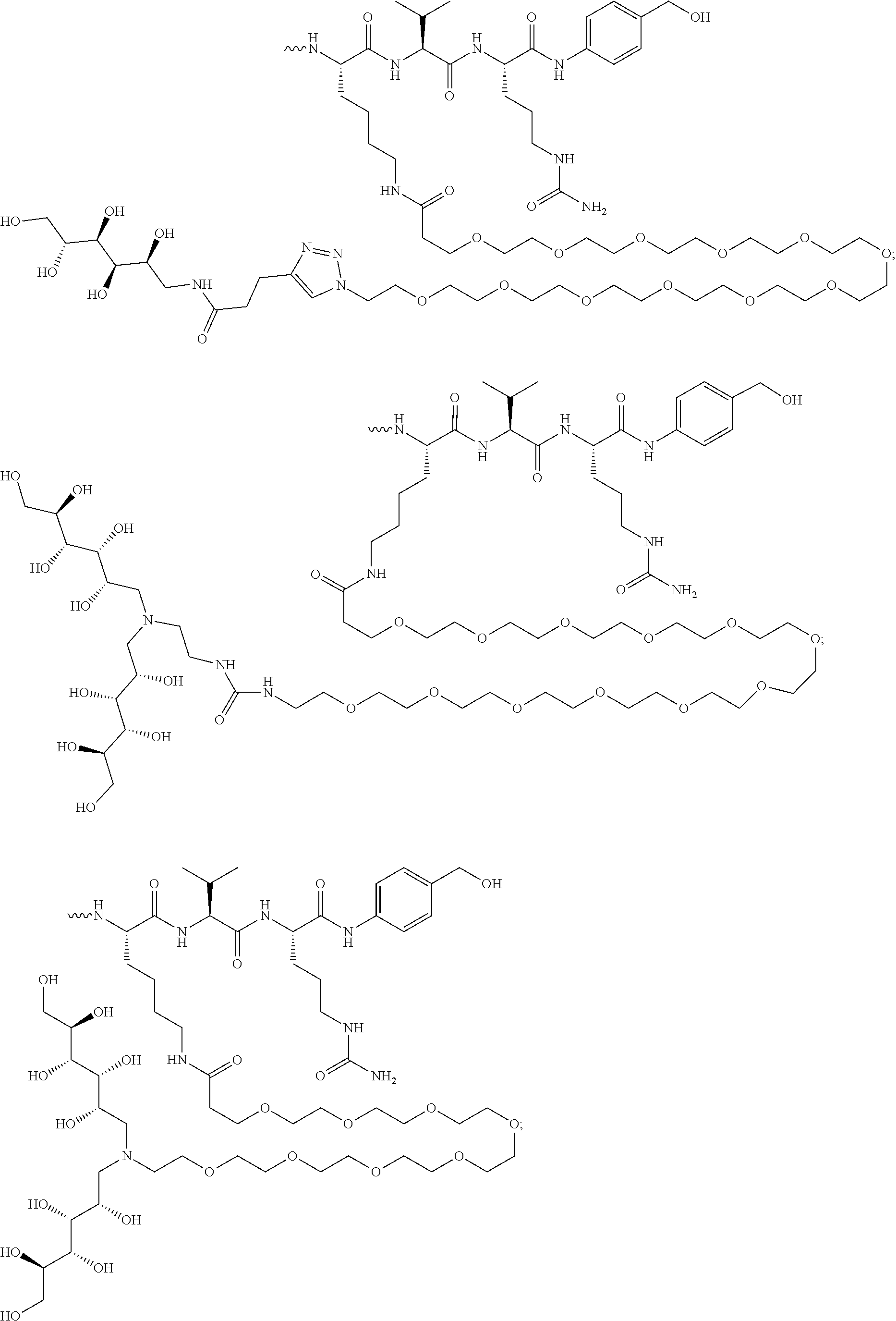

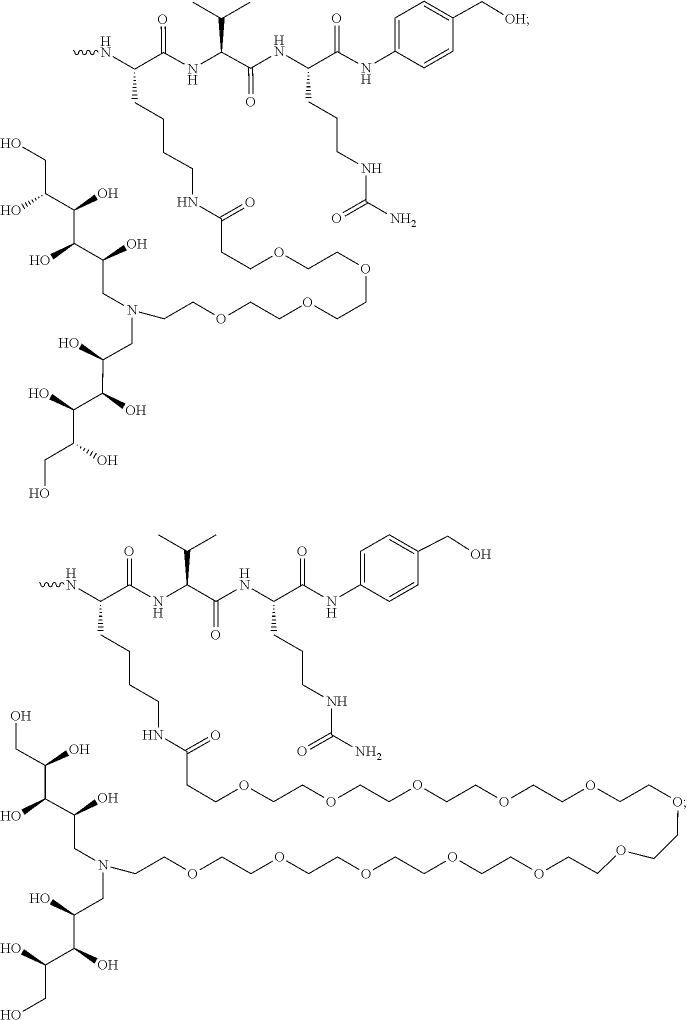

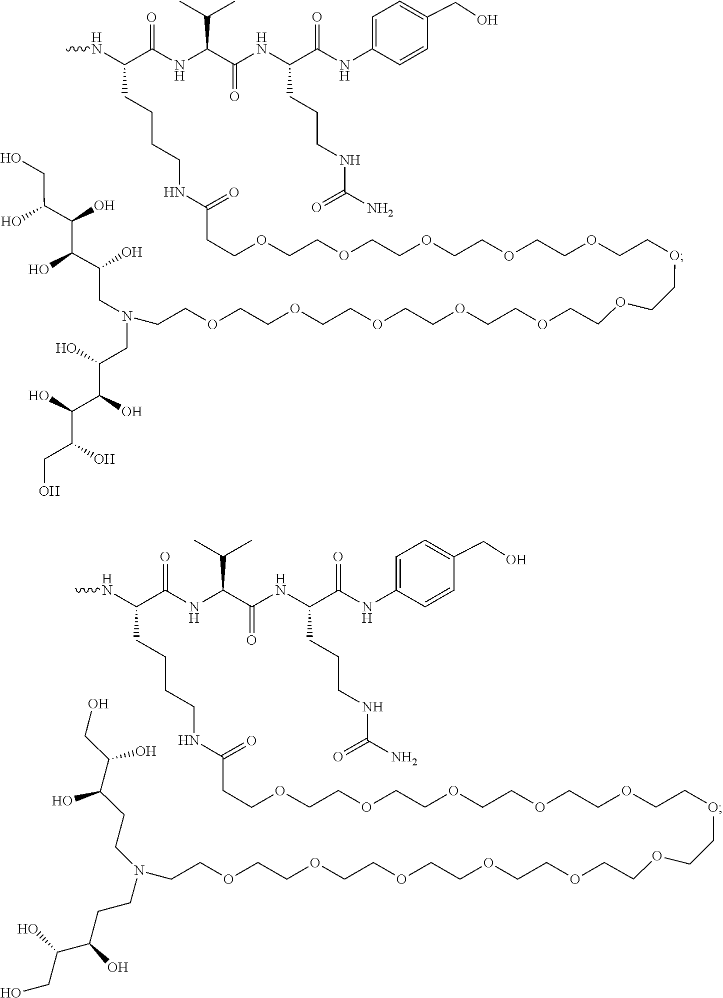

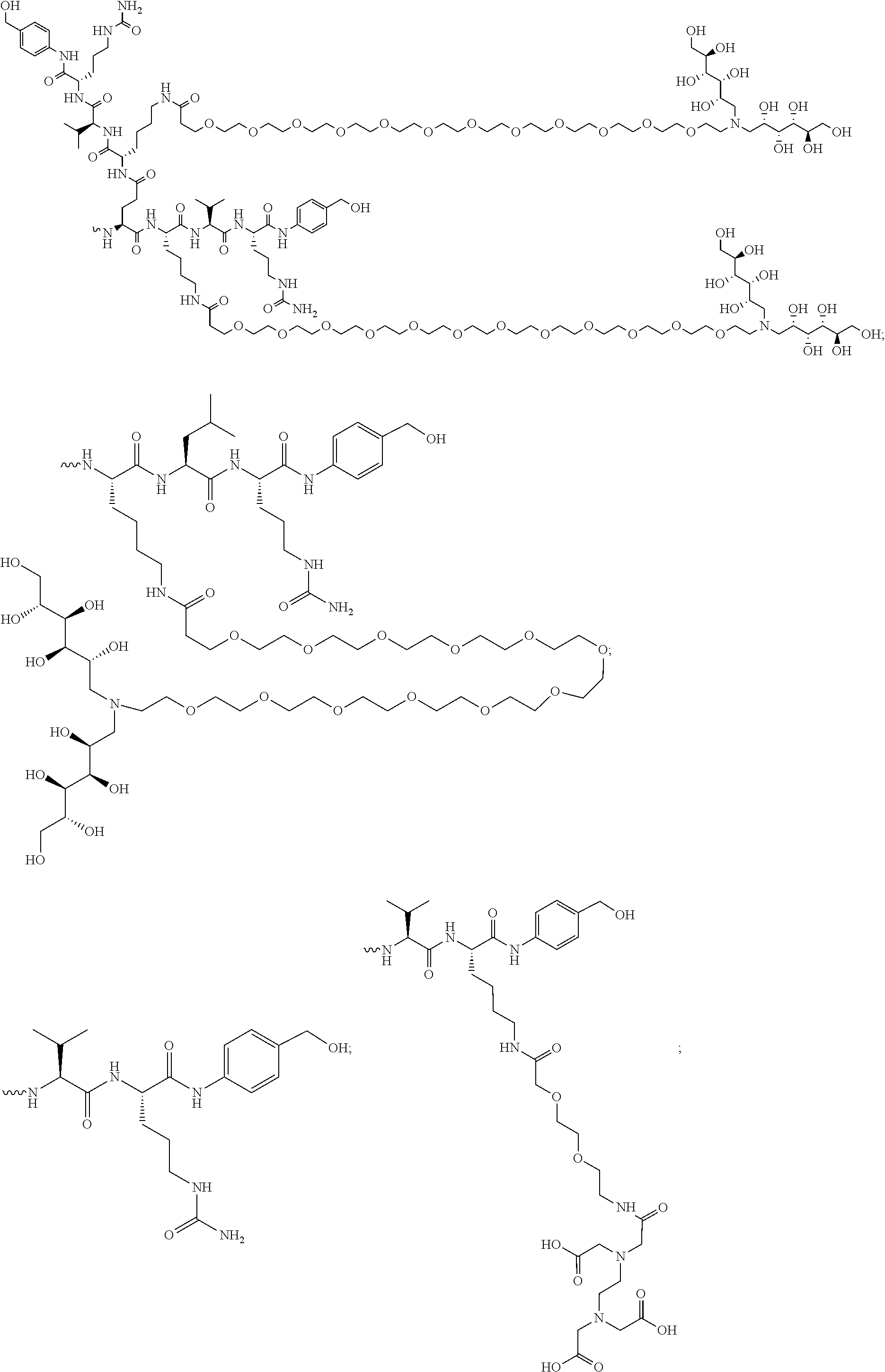

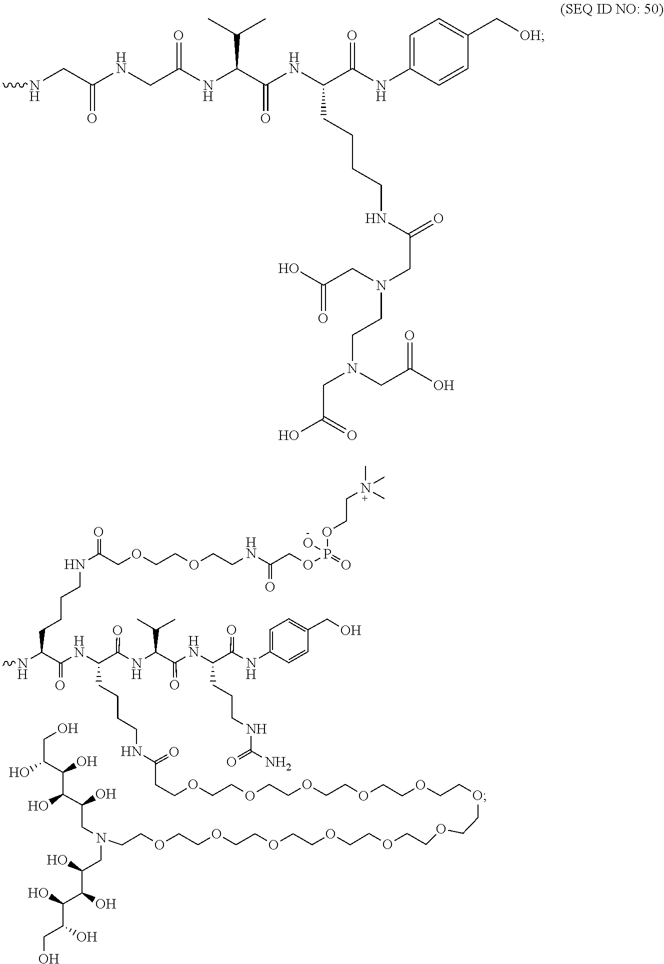

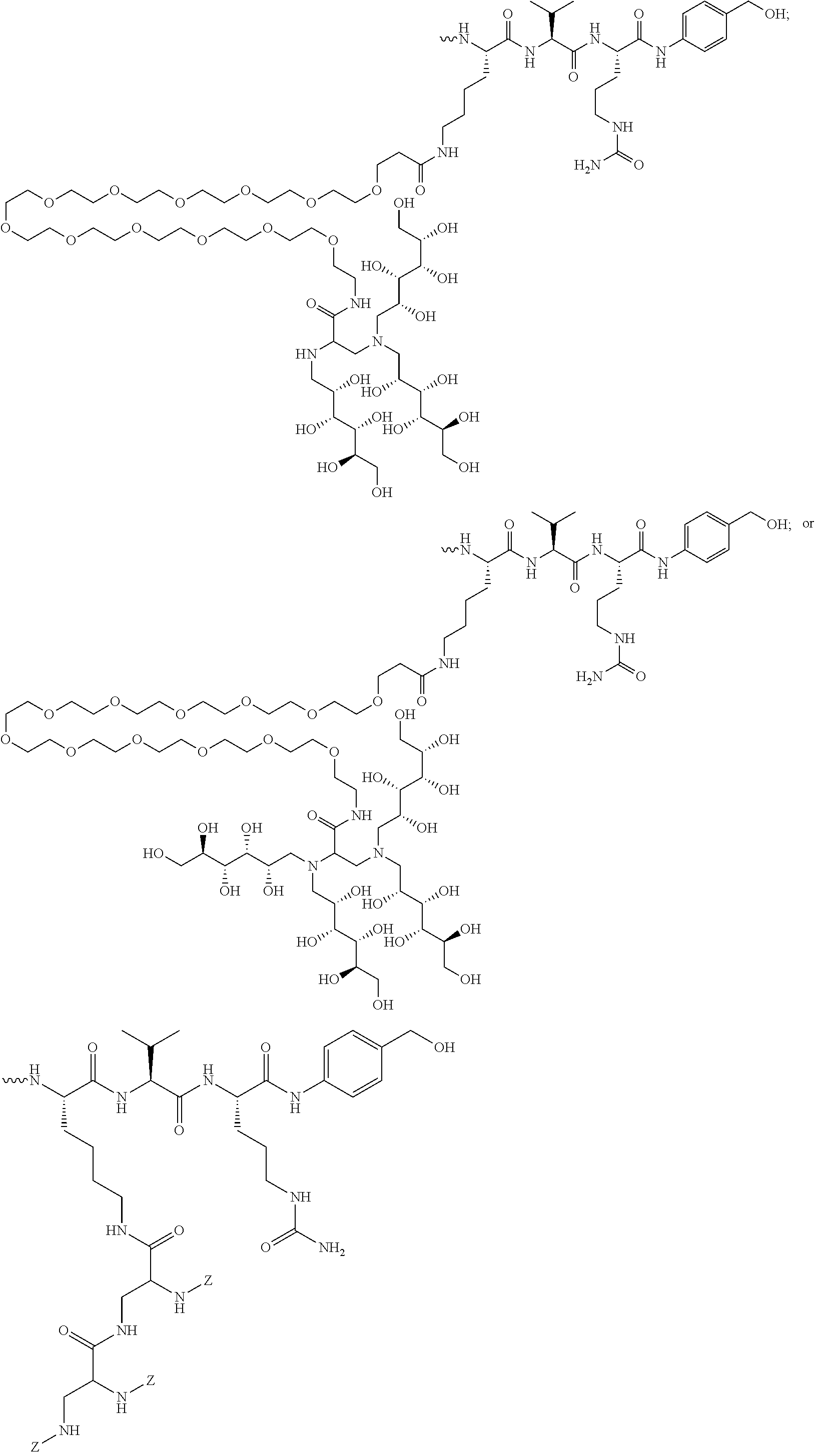

wherein each Z is attached at * and is individually selected from:

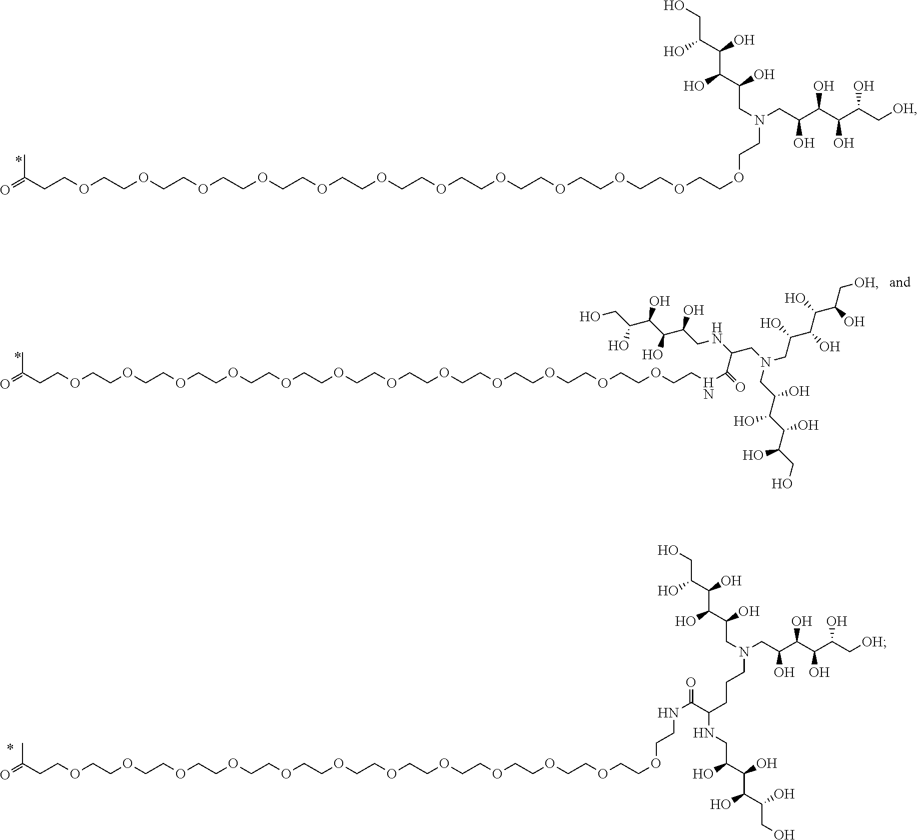

or a salt thereof, wherein the wavy line on the amino group indicates an attachment site for a Stretcher unit, and the Drug unit is attached to the benzyl alcohol (i.e., the H of benzyl alcohol is replaced with a bond to the Drug unit).

In some embodiments, a Drug-Linker intermediate ˜AA-L2-D has the following general formula:

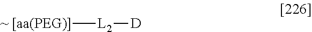

or a salt thereof, wherein [aa(PEG)] is an Amino Acid unit, in which aa is a subunit of AA selected from alpha, beta and gamma amino acids and derivatives thereof, PEG is a PEG unit attached to aa, L2 is a Linker Subunit optionally substituted with at least one Sugar unit, PEG unit, Carboxyl unit or a combination thereof, L2 is attached to AA, D is a Drug unit, and the wavy line (˜) indicates an attachment site for a Stretcher unit. In some embodiments, aa is an amino acid selected from glycine, lysine and glutamate. In some embodiments, aa is lysine. In some embodiments, Linker Subunit L2 is a cleavable linker subunit.

Exemplary embodiments of such a Drug-Linker intermediate include the following:

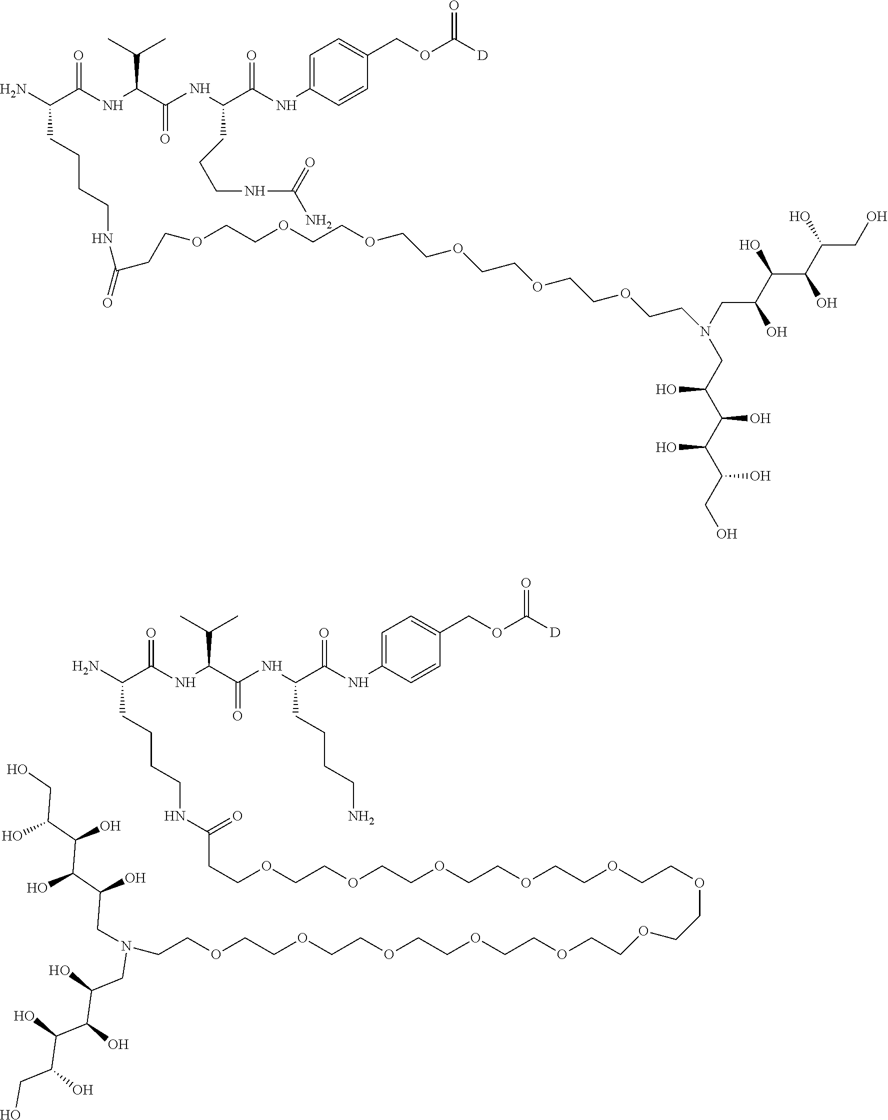

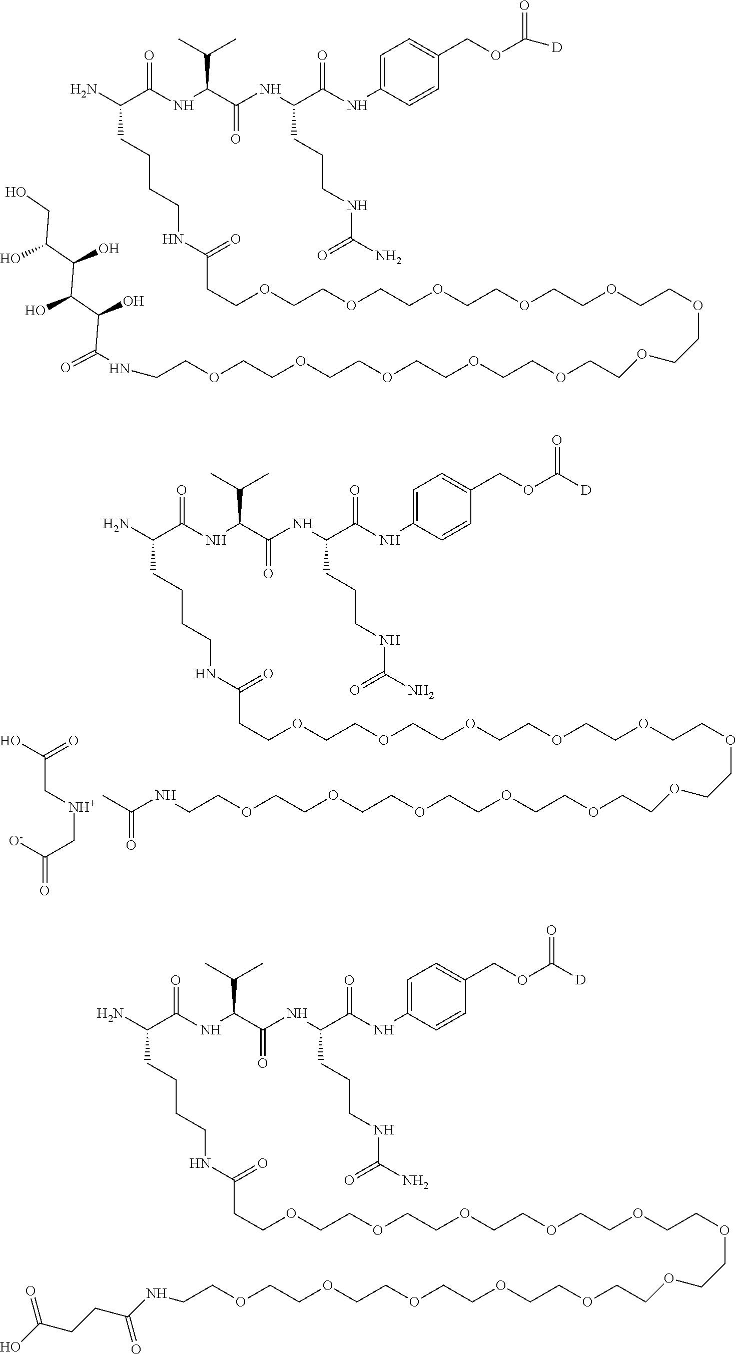

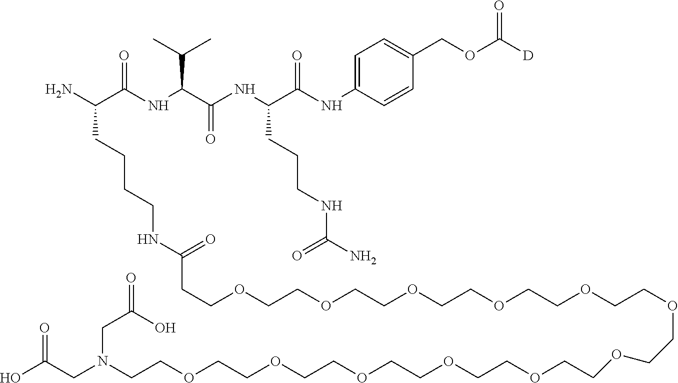

or a salt thereof, wherein the amino group on the left side of the molecule is the attachment site for the Stretcher unit.

In some embodiments, a Linker intermediate ˜L2≈ has the following general formula:

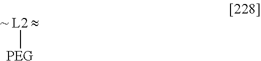

or a salt thereof, wherein an Amino Acid unit is absent, L2 is a Linker Subunit, PEG is a PEG unit attached to L2, the wavy (˜) line indicates an attachment site for a Stretcher unit, and the double wavy (≈) line indicates an attachment site for a Drug unit. In some embodiments, the PEG unit is attached to an amino acid selected from lysine, glutamate and citrulline. In some embodiments, Linker Subunit L2 is a cleavable linker subunit.

An exemplary embodiment of such a Linker intermediate includes the following:

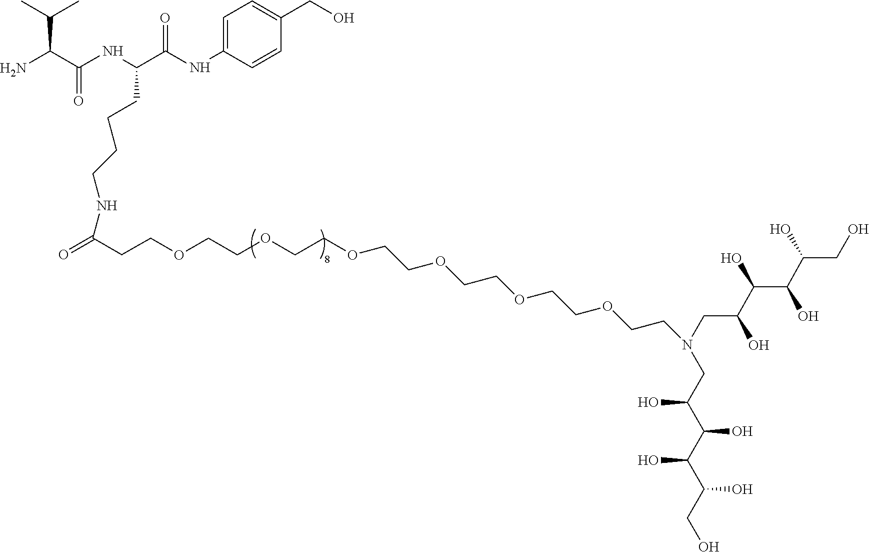

or a salt thereof, wherein the amino group on the left side of the molecule is the attachment site for the Stretcher unit.

In some embodiments, a Drug-Linker intermediate ˜L2-D has the following general formula:

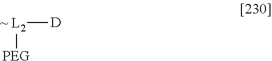

or a salt thereof, wherein an Amino Acid unit is absent, L2 is a Linker Subunit, D is a Drug unit, PEG is a PEG unit attached to L2, the wavy line (˜) indicates an attachment site for a Stretcher unit or an Amino Acid unit. In some embodiments, the PEG unit is attached to an amino acid selected from lysine, glutamate and citrulline. In some embodiments, Linker Subunit L2 is a cleavable linker subunit.

An exemplary embodiment of such a Drug-Linker intermediate includes the following:

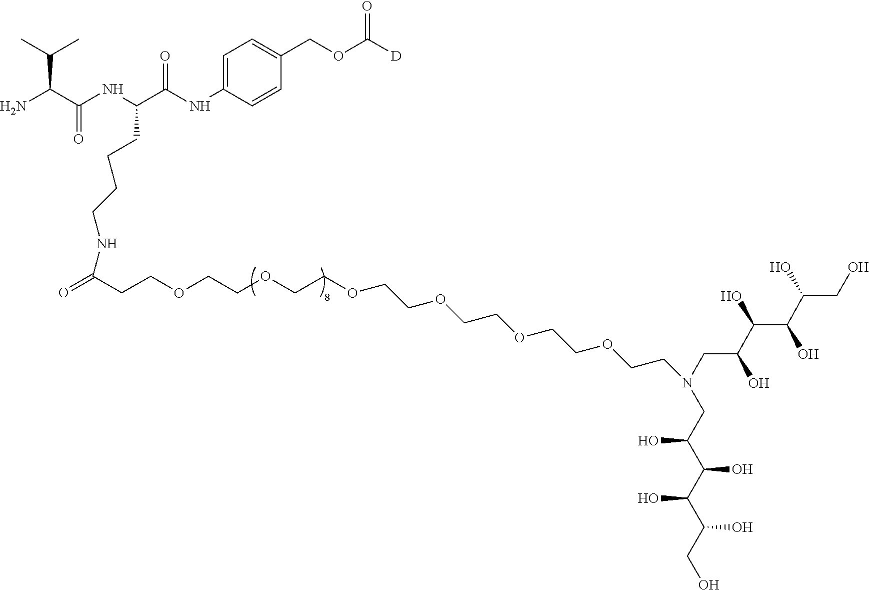

or a salt thereof, wherein the amino group on the left side of the molecule is the attachment site for the Stretcher unit.

In some embodiments, a Linker intermediate ˜AA-L2≈ has the following general formula:

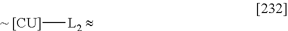

or a salt thereof, wherein [CU] is an Amino Acid unit, in which CU is a Carboxyl unit, L2 is a Linker Subunit optionally substituted with at least one Sugar unit, PEG unit, Carboxyl unit or a combination thereof, L2 is attached to AA, and the wavy (˜) line indicates an attachment site for a Stretcher unit and the double wavy (≈) indicates an attachment site for a Drug unit. In some embodiments, Linker Subunit L2 is a cleavable linker subunit.

Exemplary embodiments of such a Linker intermediate include the following:

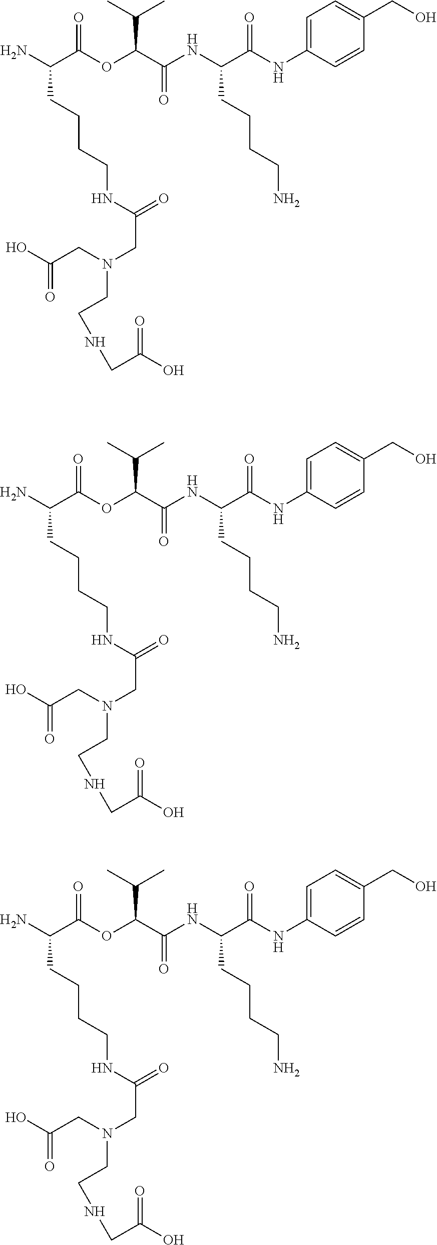

or a salt thereof, wherein the amino group on the left side of the molecule is the attachment site for the Stretcher unit and the Drug unit is attached to the benzyl alcohol (i.e., the H is removed from the benzyilic alcohol and a bond formed between the benzylic oxygen and the Drug unit).

In some embodiments, a Drug-Linker intermediate ˜AA-L2-D has the following general formula:

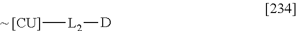

or a salt thereof, wherein [CU] is an Amino Acid unit, in which CU is a Carboxyl unit, L2 is a Linker Subunit optionally substituted with at least one Sugar unit, PEG unit, Carboxyl unit or a combination thereof, L2 is attached to AA, D is a Drug unit, and the wavy line (˜) indicates an attachment site for a Stretcher unit. In some embodiments, Linker Subunit L2 is a cleavable linker subunit.

Exemplary embodiments of such a Drug-Linker intermediate include the following:

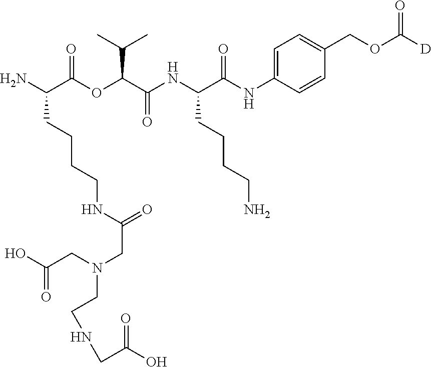

or a salt thereof, wherein the amino group on the left side of the molecule is the attachment site for the Stretcher unit.

In some embodiments, a Drug-Linker intermediate ˜L2-D has the following general formula:

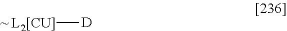

or a salt thereof, wherein L2 is a Linker Subunit comprising a Carboxyl unit [CU], D is a Drug unit, and the wavy line (˜) indicates an attachment site for an Amino Acid unit or a Stretcher unit. In some embodiments, Linker Subunit L2 is a cleavable linker subunit.

Exemplary embodiments of such a Drug-Linker intermediate include the following

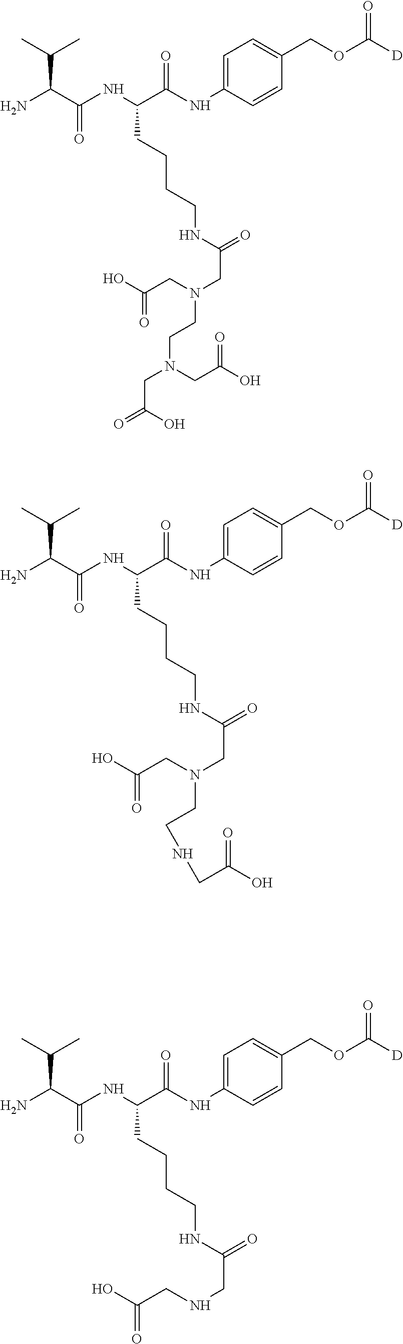

or a salt thereof, wherein the amino group on the left side of the molecule is the attachment site for an Amino Acid unit or a Stretcher unit.

In some embodiments of a Linker or Linker Intermediates of previous formulae [190] to [236], Linker Subunit L2 is a cleavable linker subunit.

In some embodiments of a Linker or Linker Intermediates of previous formulae (190) to [236], Linker Subunit L2 is a cleavable linker subunit having a peptide selected from valine-citrulline, phenylalanine-lysine, alanine-lysine and glycine-glycine-phenylalanine-lysine (SEQ ID NO: 48).

Attachment of Drug-Linkers to Antibodies, Antigen Binding Portions and Other Binding Agents (including Non-Antibody Scaffolds)

Techniques for attaching Drug unit(s) to Targeting units (such as antibodies or antigen binding portions thereof or non-antibody scaffolds) via linkers are well-known in the art. See, e.g., Alley et al., Current Opinion in Chemical Biology 2010 14:1-9; Senter, Cancer J., 2008, 14(3):154-169. In some embodiments, a Linker is first attached to a Drug unit (e.g., a cytotoxic agent(s), immune modulatory agent or other agent) and then the Drug-Linker(s) is attached to the Targeting unit (e.g., an antibody or antigen binding portion thereof or non-antibody protein scaffold). In some embodiments, a Linker(s) is first attached to a Targeting unit (e.g., an antibody or antigen binding portion thereof or non-antibody protein scaffold), and then a Drug unit is attached to a Linker. In the following discussion, the term Drug-Linker is used to exemplify attachment of Linkers or Drug-Linkers to Targeting units; the skilled artisan will appreciate that the selected attachment method can be determined according to Linker and the Drug unit. In some embodiments, a Drug unit is attached to a Targeting unit via a Linker in a manner that reduces the activity of the Drug unit until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of a Targeting unit (e.g., an antibody or antigen binding portion thereof or non-antibody protein scaffold) with a bivalent Linker to form a Targeting unit-Linker intermediate via a covalent bond, followed by reaction with a Drug unit; and (2) reaction of a nucleophilic group of a Drug unit with a bivalent Linker, to form Drug-Linker, via a covalent bond, followed by reaction with a nucleophilic group of a Targeting unit. Exemplary methods for preparing conjugates via the latter route are described in U.S. Pat. No. 7,498,298, which is expressly incorporated herein by reference.

Nucleophilic groups on Targeting units such as antibodies, antigen binding portions and other binding agents (including non-antibody scaffolds) include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on Linkers including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain Targeting units, such as antibodies (and antigen binding portions and other binding agents (including non-antibody scaffolds)) have reducible interchain disulfides, i.e., cysteine bridges. Antibodies (and antigen binding portions and other binding agents (including non-antibody scaffolds)) may be made reactive for conjugation with Linkers by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into Targeting units such as antibodies (and antigen binding portions and other binding agents (including non-antibody scaffolds)) through modification of lysine residues, e.g., by reacting lysine residues with 2-iminothiolane (Traut's reagent), resulting in conversion of an amine into a thiol. Reactive thiol groups may also be introduced into a Targeting unit (such as an antibody and antigen binding portions and other binding agents (including non-antibody scaffolds)) by introducing one, two, three, four, or more cysteine residues (e.g., by preparing antibodies, antigen binding portions and other binding agents (including non-antibody scaffolds) comprising one or more non-native cysteine amino acid residues).

Conjugates may also be produced by reaction between an electrophilic group on a Targeting unit, such as an aldehyde or ketone carbonyl group, with a nucleophilic group on a Linker reagent. Useful nucleophilic groups on a linker reagent include, but are not limited to, hydrazide, oxime, amino, hydrazine, thiosemicarbazone, hydrazine carboxyl, and arylhydrazide. In an embodiment, an antibody (or antigen binding portion thereof or other binding agent (including non-antibody scaffolds)) is modified to introduce electrophilic moieties that are capable of reacting with nucleophilic substituents on a Linker. In another embodiment, the sugars of glycosylated antibodies may be oxidized, e.g. with periodate oxidizing reagents, to form aldehyde or ketone groups which may react with the amine group of a Linker. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent (including non-antibody scaffolds)) that can react with appropriate groups on the Linker (see, e.g., Hermanson, Bioconjugate Techniques). In another embodiment, Targeting units such as antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a Linker.

Exemplary nucleophilic groups on a Drug unit, such as a cytotoxic agent, include, but are not limited to: amine, thiol, hydroxyl, hydrazide, oxime, hydrazine, thiosemicarbazone, hydrazine carboxyl, and arylhydrazide groups capable of reacting to form covalent bonds with electrophilic groups on a Linker(s) including: (i) active esters such as NHS esters, HOBt esters, haloformates, and acid halides; (ii) alkyl and benzyl halides such as haloacetamides; (iii) aldehydes, ketones, carboxyl, and maleimide groups.

In some embodiments, a Drug-Linker is attached to an interchain cysteine residue(s) of an antibody (or antigen binding portion thereof or other binding agent (including non-antibody scaffolds)). See, e.g., WO2004/010957 and WO2005/081711. In such embodiments, the Linker typically comprises a maleimide group for attachment to the cysteine residues of an interchain disulfide. In some embodiments, a Linker or Drug-Linker is attached to a cysteine residue(s) of an antibody or antigen binding portion thereof as described in U.S. Pat. Nos. 7,585,491 or 8,080,250. The drug loading of the resulting conjugate typically ranges from 1 to 8 or 1 to 16.

In some embodiments, a Linker or Drug-Linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents (including non-antibody scaffolds).

In some embodiments, a Drug-Linker(s) is attached to an engineered cysteine residue at an Fc residue other than an interchain disulfide. In some embodiments, a Drug-Linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a Linker or Drug-Linker(s) is attached to one or more introduced cysteine residues of an antibody (or antigen binding portion thereof or other binding agent (including non-antibody scaffolds)) as described in WO2006/034488, WO2011/156328 and/or WO2016040856.

In some embodiments, an exemplary substitution for site specific conjugation using bacterial transglutaminase is N297S or N297Q of the Fc region. In some embodiments, a Linker or Drug-Linker(s) is attached to the glycan or modified glycan of an antibody or antigen binding portion or a glycoengineered antibody (or other binding agent (including non-antibody scaffolds)). See, e.g., WO2017/147542, WO2020/123425, WO2020/245229, WO2014/072482; WO2014//065661, WO2015/057066 and WO2016/022027; the disclosure of which are incorporated by reference herein.

In some embodiments, a Linker or Drug-Linker is attached to an antibody, antigen binding portion or other binding agent (including non-antibody scaffolds) via Sortase A linker. A Sortase A linker can be created by a Sortase A enzyme fusing an LPXTG recognition motif (SEQ ID NO: 5) to an N-terminal GGG motif to regenerate a native amide bond.

In some embodiments, a Linker or Drug-Linker is attached to an antibody, antigen binding portion or other binding agent (including non-antibody scaffolds) using SMARTag Technology, in which a bioorthogonal aldehyde handle is introduced through the oxidation of a cysteine residue, embedded in a specific peptide sequence (CxPxR), to an aldehyde-bearing formylglycine (fGly). This enzymatic modification is carried out by the formylglycine-generating enzyme (FGE). See, e.g., Liu et al., Methods Mol. Biol. 2033:131-147 (2019).

In some embodiments, a Linker or Drug-Linker is attached to an antibody, antigen binding portion or other binding agent (including non-antibody scaffolds) using cysteine conjugation with quaternized vinyl- and alkynyl-pyridine reagents. See, e.g., Matos et al., Angew Chem. Int. Ed. Engl. 58:6640-6644 (2019).

In other embodiments, a Linker or Drug-Linker is attached to an antibody, antigen binding portion or other binding agent (including non-antibody scaffolds) using bis-maleimide, C-lock, or K-lock methodologies.

### Pharmaceutical Formulations

Other aspects of the conjugates relate to compositions comprising active ingredients, including any of the conjugates described herein. In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A conjugate can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a conjugate). The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain the active ingredients (e.g., a conjugate) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a conjugate can be a lyophilisate.

In some embodiments, a syringe comprising a therapeutically effective amount of a conjugate is provided.

### Treatment of Cancer

In some embodiments, the conjugates as described herein can be used in a method(s) comprising administering a conjugate as described herein to a subject in need thereof, such as a subject having cancer.

In some embodiments, provided are methods of treating cancer comprising administering a conjugate In some embodiments, the subject is in need of treatment for a cancer and/or a malignancy. In some embodiments, the method is for treating a subject having a cancer or malignancy.

The methods described herein include administering a therapeutically effective amount of a conjugate to a subject having a cancer or malignancy. As used herein, the phrases “therapeutically effective amount”, “effective amount” or “effective dose” refer to an amount of a conjugate that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

It is contemplated that the methods herein reduce tumor size or tumor burden in the subject, and/or reduce metastasis in the subject. In various embodiments, tumor size in the subject is decreased by about 25⁻⁵⁰%, about 40⁻⁷⁰% or about 50⁻⁹⁰% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

In some embodiments, the subject is in need of treatment for a cancer and/or a malignancy with an anti-FOLRI conjugate. In a specific embodiment, the anti-FOLRI conjugate contains antibody F131 (VH SEQ ID NO: 26 and VL SEQ ID NO: 27). In a specific embodiment, the anti-FOLR1 conjugate comprises an F131 antibody and an LD038 Linker-Drug. In some embodiments, the subject is in need of treatment for a FOLR1+ cancer or a FOLR1+ malignancy, such as for example, lung cancer, non-small cell lung cancer, ovarian cancer, breast cancer, uterine cancer, cervical cancer, endometrial cancer, pancreatic cancer, and renal cell cancer. In some embodiments, the method is for treating a subject having a FOLR1+ cancer or malignancy. In some embodiments, the method is for treating lung cancer in a subject. In some embodiments, the method is for treating non-small cell lung cancer in a subject. In some embodiments, the method is for treating breast cancer in a subject. In some embodiments, the method is for treating ovarian cancer in a subject. In some embodiments, the method is for treating cervical cancer in a subject. In some embodiments, the method is for treating endometrial cancer in a subject. In some embodiments, the method is for treating renal cell cancer in a subject. In some embodiments, the method is for treating uterine cancer in a subject. In some embodiments, the method is for treating pancreatic cancer in a subject.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a cancer and in need of treatment, but need not have already undergone treatment for the cancer. In some embodiments, a subject can also be one who has not been previously diagnosed as having a cancer in need of treatment. In some embodiments, a subject can be one who exhibits one or more risk factors for a condition or one or more complications related to a cancer or a subject who does not exhibit risk factors. A “subject in need” of treatment for a cancer particular can be a subject having that condition or diagnosed as having that condition. In other embodiments, a subject “at risk of developing” a condition refers to a subject diagnosed as being at risk for developing the condition or at risk for having the condition again.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a conjugate as described herein to a subject by a method or route which results in binding of the conjugate to cancer cells or malignant cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate can be administered in a unit dose. The term “unit dose” when used in reference to a pharmaceutical composition refers to physically discrete units suitable as unitary dosage for the subject, each unit containing a predetermined quantity of active material (e.g., conjugate), calculated to produce the desired therapeutic effect in association with the required physiologically acceptable diluent, i.e., carrier, or vehicle.

### Treatment of Autoimmune Disease

In some embodiments, the conjugates as described herein can be used in a method(s) comprising administering a conjugate to a subject in need thereof, such as a subject having an autoimmune disease.

In some embodiments, provided are methods of treating an autoimmune disease comprising administering a conjugate as described herein. In some embodiments, the subject is in need of treatment for an autoimmune disease. The methods described herein include administering a therapeutically effective amount of a conjugate to a subject having an autoimmune disease. As used herein, the phrase “therapeutically effective amount”, “effective amount” or “effective dose” refers to an amount of a conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The term “autoimmune disease” refers to an immunological disorder characterized by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease, glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune diseases. In addition, the methods described herein can be used to treat domesticated animals and/or pets. A subject can be male or female. In certain embodiments, the subject is a human.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in autoimmune cells in the subject, alleviation of one or more symptom(s), diminishment of extent of the deficit, stabilized (i.e., not worsening) state of an autoimmune disease, delay or slowing of progression of an autoimmune disease, and an increased lifespan as compared to that expected in the absence of treatment. As used herein, the term “administering,” refers to providing a conjugate as described herein to a subject by a method or route which results in binding of the conjugate to target autoimmune cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate thereof can be administered in a unit dose. The term “unit dose” when used in reference to a pharmaceutical composition refers to physically discrete units suitable as unitary dosage for the subject, each unit containing a predetermined quantity of active material (e.g., a conjugate), calculated to produce the desired therapeutic effect in association with the required physiologically acceptable diluent, i.e., carrier, or vehicle.

In some embodiments, a conjugate, or a pharmaceutical composition of any of these, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a conjugate or a pharmaceutical composition thereof to the subject, wherein the conjugate specifically binds to target autoimmune cells; wherein the treatment outcome of the subject is improved, as compared to administration of the immunotherapy alone. In some embodiments, the conjugate thereof as described herein. In some embodiments, an improved treatment outcome is a decrease in disease progression, an alleviation of one or more symptoms, or the like.

The present invention is further illustrated by the following embodiments which should not be construed as limiting.

1. A Linker intermediate having the following formula (V):

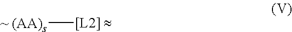

or a salt thereof, wherein:
   AA is an Amino Acid unit having from 1 to 12 amino acid subunits;
   s is 0 or 1;
   L2 is a Linker Subunit having from 1 to 4 attachment sites for a Drug unit; and
   each wavy (˜) line indicates an attachment site for a Stretcher Unit and the
   double wavy (≈) line indicates an attachment site for a Drug Unit,
wherein at least one Polar unit is present within the Amino Acid unit, the Linker Subunit, or both, and wherein the Polar unit(s) is selected from Sugar units, PEG units, Carboxyl units, and combinations thereof.
   2. A Linker having the following formula (I):

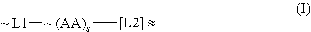

or a salt thereof, wherein:
L1 is a Stretcher unit having an attachment site for a Targeting unit;
AA is an Amino Acid unit having from 1 to 12 subunits;
s is 0 or 1;
L2 is a Linker Subunit having from 1 to 4 attachment sites for a Drug unit;
the wavy (˜) line indicates an attachment site for the Targeting unit, and the
double wavy (≈) line indicates an attachment site for a Drug unit;
wherein at least one Polar unit is present within the Amino Acid unit, the Linker Subunit, or both, and wherein the Polar unit(s) is selected from Sugar units, PEG units, Carboxyl units, and combinations thereof.
   3. The Linker intermediate or Linker of the previous embodiments, wherein the Sugar unit has the following formula:

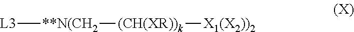

or a salt thereof, wherein:
   each X is independently selected from NH or 0;
   each R is independently selected from hydrogen, acetyl, a monosaccharide, a disaccharide, and a polysaccharide;
   each X, is independently selected from CH₂ and C(O);
   each X₂ is independently selected from H, OH and OR;
   k is 1 to 10; and
   L3 has the following general formula (XI):

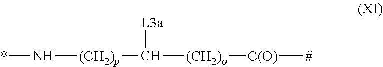

or a salt thereof, wherein:
   L3a is selected from C₁-C₁₀ alkylene and polyethylene glycol having from 1 to 24 ethylene glycol subunits;
   p and o are independently 0 to 2;
   each * and each # indicate an attachment site for another subunit of an Amino Acid unit (AA), the Linker subunit L2, or the Stretcher unit (L1); and
   L3a is covalently bound to the N atom marked with a ** in formula (X).
      4. The Linker intermediate or Linker of any of the previous embodiments, wherein the Sugar unit has the formula selected from:

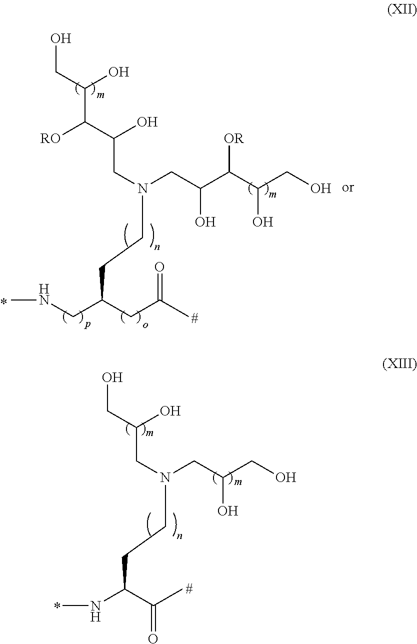

or a salt thereof, wherein:
   each R is independently selected from hydrogen, a monosaccharide, a disaccharide and a polysaccharide;
   p and o are independently 0 to 2;
   m is 1 to 8;
   n is 0 to 4; and
   each * and each # indicate an attachment site for another subunit of the Amino Acid unit (AA), the Linker subunit L2, or the Stretcher unit (L1).
      5. The Linker intermediate or Linker of any of the previous embodiments, wherein the PEG unit has a formula selected from:
      (a)

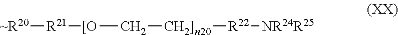

or a salt thereof, wherein:
   R²⁰ is a functional group for attachment to a subunit of the Amino Acid unit or a portion of the Linker Subunit L2;
   R²¹ and R²² are each, independently, optional C₁-C₃ alkylene;
   R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); or —NR²⁴R²⁵ together from a C₃-C₈ heterocycle; the wavy line (˜) indicates the attachment site to R²⁰; and n20 is 1 to 26;
   or
(b)

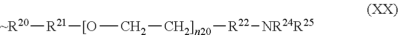

or a salt thereof, wherein:
R²⁰ is a functional group for attachment to a subunit of the Amino Acid unit or a portion of the Linker Subunit L2;
R²¹ and R²² are each, independently, optional C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is a polyethylene glycol, optionally having 1 to 24 ethylene glycol subunits;
the wavy line (˜) indicates the attachment site to R²⁰; and n20 is 1 to 26;
or
(c)

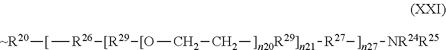

or a salt thereof, wherein:
R²⁰ is a functional group for attachment to a subunit of an Amino Acid unit and/or a portion of a Linker Subunit L2;
R²⁶ and R²⁷ are each optional and are, independently, selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a Sugar unit of formula (XII) or (XIII); and polyethylene glycol, optionally having 1 to 24 ethylene glycol subunits; or —NR²⁴R²⁵ together from a C₃-C₈ heterocycle;
the wavy line (˜) indicates the attachment site to R²⁰
n20 is 1 to 26;
n21 is 1 to 4; and.
n27 is 1 to 4.
   6. The Linker intermediate or Linker of embodiment 5, wherein both R²⁴ and R²⁵ are not H.
   7. The Linker intermediate or Linker of any of embodiments 5 to 6, wherein R²⁴ and R²⁵ are each independently selected from a H and polyhydroxyl group, provided that R²⁴ and R²⁵ are not both H.
   8. The Linker intermediate or Linker of any of embodiments 5 to 7, wherein the polyhydroxyl group is a linear monosaccharide, optionally selected from a C₆ or C₅ sugar, sugar acid or amino sugar.
   9. The Linker intermediate or Linker of embodiment 8, wherein:
the C₆ or C₅ sugar is selected from glucose, ribose, galactose, mannose, arabinose, 2-deoxyglucose, glyceraldehyde, erythrose, threose, xylose, lyxose, allose, altrose, gulose, idose talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.
   10. The Linker intermediate or Linker of any of embodiments 5 to 9, wherein the PEG unit is selected from the following, or a salt thereof:

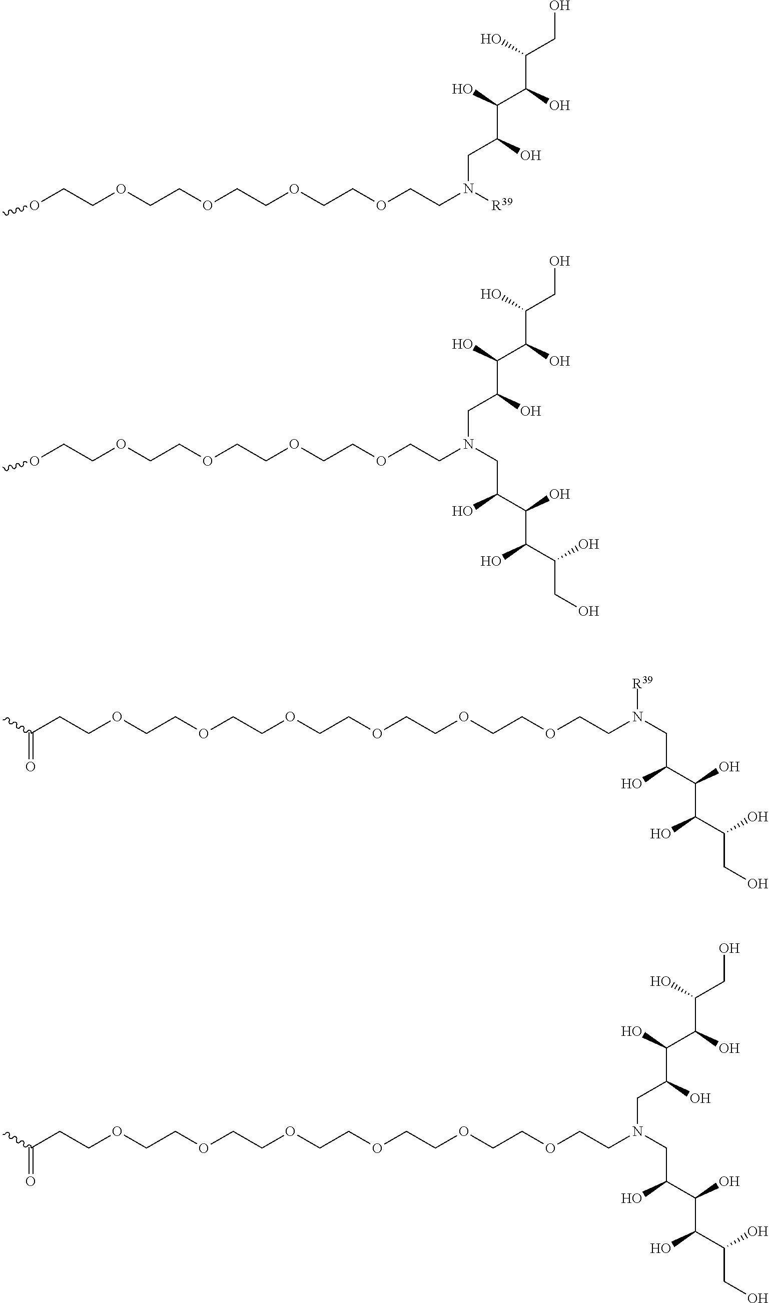

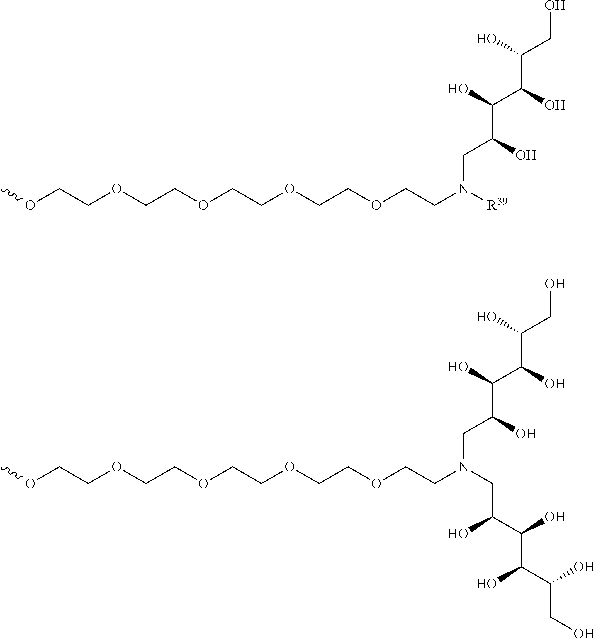

wherein R³⁹ is selected from H, a linear monosaccharide and polyethylene glycol, optionally having from 1 to 24 ethylene glycol subunits; and the wavy line at the left side indicates the attachment site to the subunit of the Amino Acid unit or the portion of the Linker subunit.
   11. The Linker intermediate or Linker of any of embodiments 5 to 6, wherein one of R²⁴ and R²⁵ is a linear monosaccharide and the other is a cyclic monosaccharide.
   12. The Linker intermediate or Linker of embodiment 11, wherein the PEG unit is selected from the following, or a salt thereof:

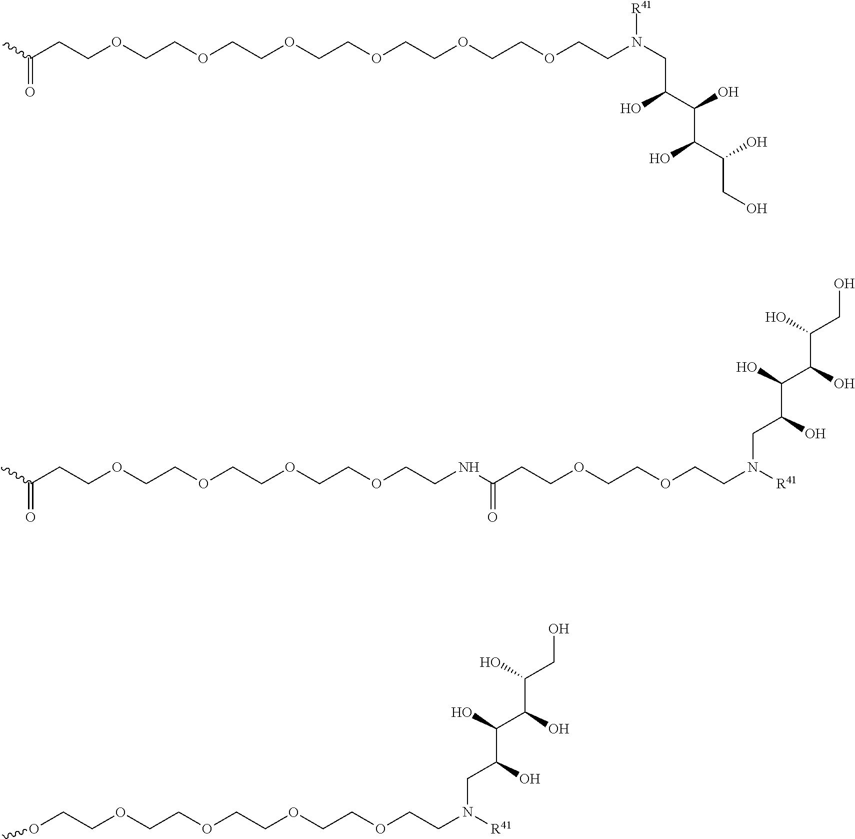

wherein R⁴¹ is a cyclic monosaccharide; and the wavy line at the left side indicates the attachment site to the subunit of the Amino Acid unit or the portion of the Linker subunit.
   13. The Linker intermediate or Linker of any of embodiments 5 to 6, wherein R²⁴ and R²⁵ are independently selected from cyclic monosaccharides, disaccharides and polysaccharides.
   14. The Linker intermediate or Linker of embodiment 13, wherein the PEG unit is selected from the following, or a salt thereof:

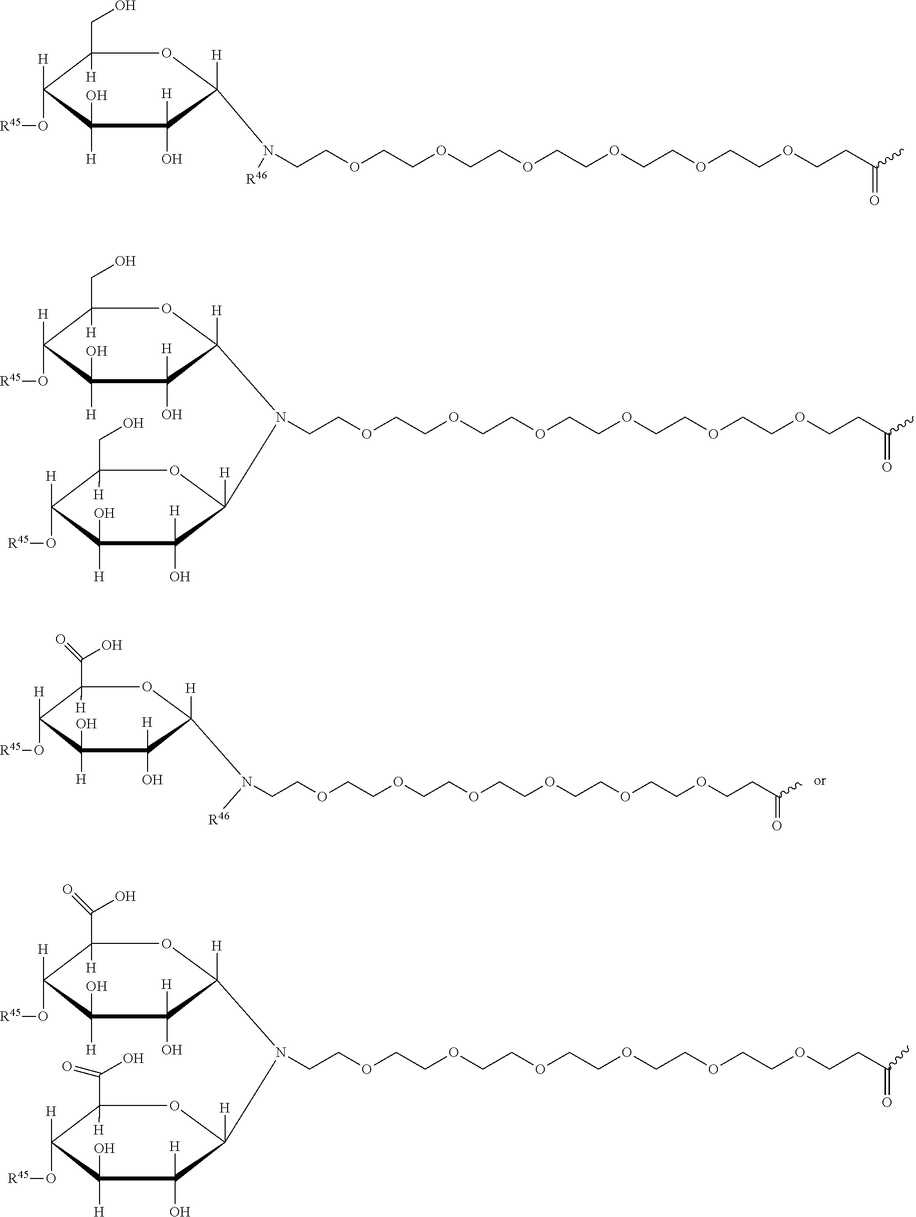

wherein each R⁴⁵ is selected from H and a monosaccharide, a disaccharide, or a polysaccharide; and R⁴⁶⁻ is selected from a cyclic monosaccharide, disaccharide, or polysaccharide; and the wavy line at the right side indicates the attachment site to the subunit of the Amino Acid unit or the portion of the Linker subunit.
   15. The Linker intermediate or Linker of any of embodiments 5 to 6, wherein R²⁴ and R²⁵ are independently selected from a linear monosaccharide and a substituted linear monosaccharide, wherein the substituted linear monosaccharide is substituted with a monosaccharide, a disaccharide or a polysaccharide.
   16. The Linker intermediate or Linker of embodiment 15, wherein the PEG unit is selected from the following, or a salt thereof:

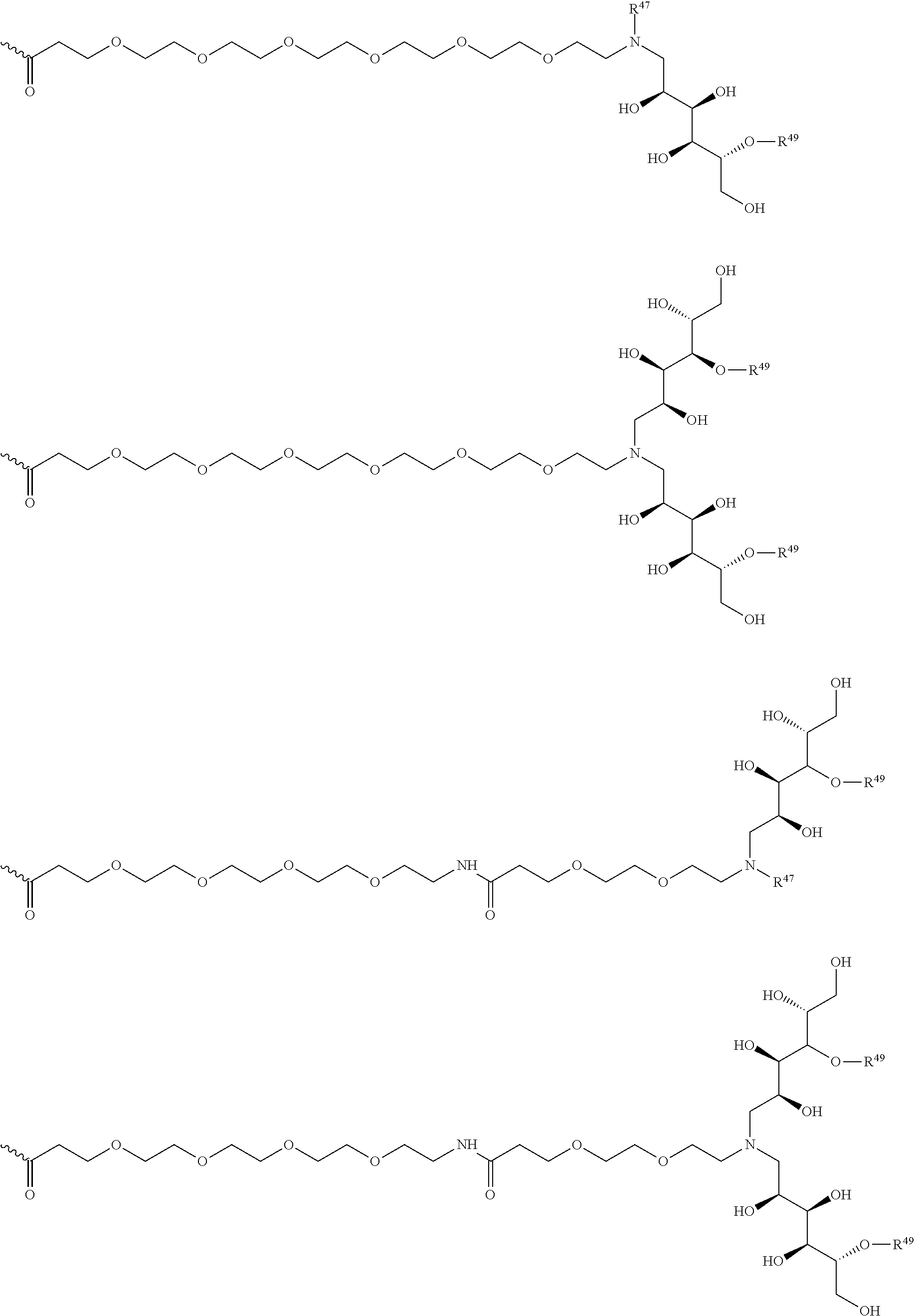

wherein R⁴⁷ is a linear monosaccharide; and each R⁴⁹ is selected from a monosaccharide, a disaccharide and a polysaccharide; and the wavy line at the left side indicates the attachment site to the subunit of the Amino Acid unit or the portion of the Linker subunit.
   17. The Linker intermediate or Linker of any of embodiments 5 to 6, wherein R²⁴ and R²⁵ are independently selected from a linear monosaccharide and a substituted monosaccharide, wherein the substituted linear monosaccharide is substituted with one or more substituents selected from alkyl, O-alkyl, aryl, O-aryl, carboxyl, ester, or amide, and optionally further substituted with a monosaccharide, disaccharide or a polysaccharide.
   18. The Linker intermediate or Linker of embodiment 17, wherein the PEG unit is selected from the following, or a salt thereof:

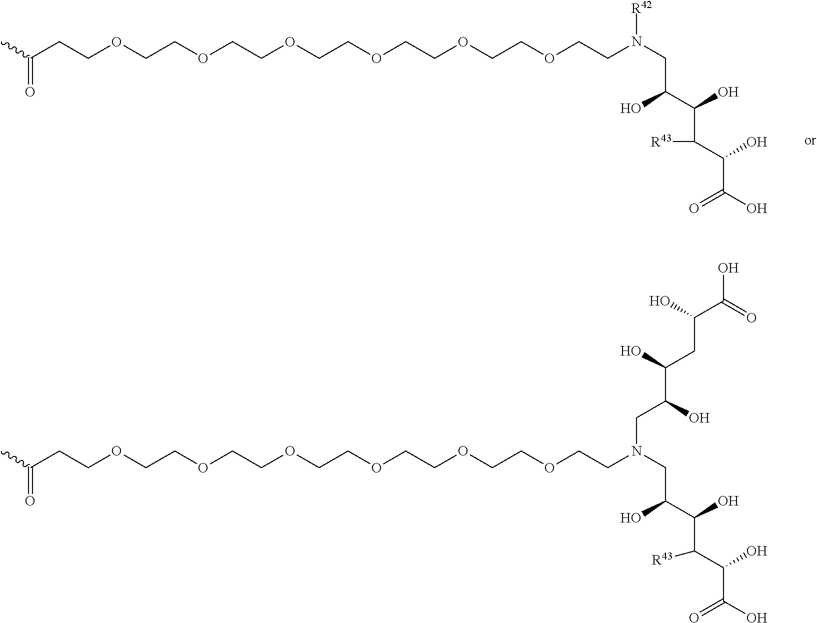

wherein each R⁴² is independently selected from a linear monosaccharide and a substituted linear monosaccharide; each R⁴³ is independently selected from alkyl, O-alkyl, aryl, O-aryl, carboxyl, ester, and amide; and the wavy line at the left side indicates the attachment site to the subunit of the Amino Acid unit or the portion of the Linker subunit.
   19. The Linker intermediate or Linker of any of embodiments 5 to 6, wherein one of R²⁴ and R²⁵ is a —C(O)-polyhydroxyl group or substituted —C(O)-polyhydroxyl group, and the other of R²⁴ and R²⁵ is a H, —C(O)-polyhydroxyl group, substituted —C(O)-polyhydroxyl group, polyhydroxyl group or substituted polyhydroxyl group; wherein the substituted —C(O)— polyhydroxyl group and polyhydroxyl group are substituted with a monosaccharide, a disaccharide, a polysaccharide, alkyl, —O-alkyl, aryl, carboxyl, ester, or amide.
   20. The Linker intermediate or Linker of embodiment 19, wherein the PEG unit is selected from the following, or a salt thereof:

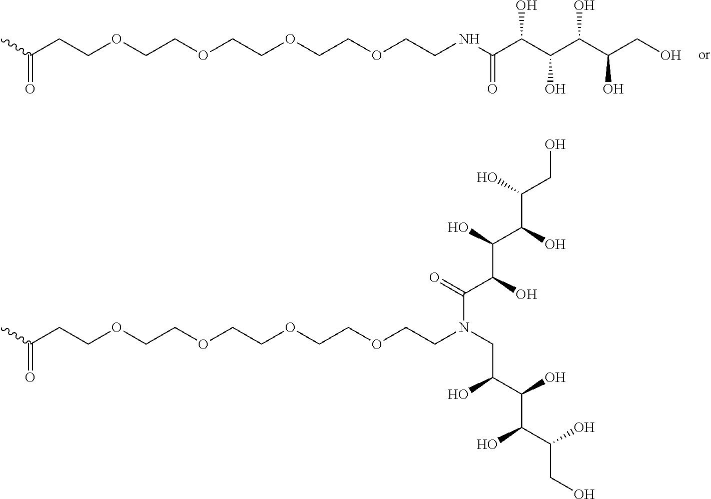

wherein the wavy line at the left side indicates the attachment site to the subunit of the Amino Acid unit or the portion of the Linker subunit.
   21. The Linker intermediate or Linker of any of embodiments 5 to 6, wherein R²⁴ and R²⁵ are independently selected from a H, substituted —C₁-C₈ alkyl, substituted —C₁-C₄ alkyl or substituted —C₁-C₃ alkyl; provided that both R²⁴ and R²⁵ are not H; wherein substituted —C₁-C₈ alkyl, —C₁-C₄ alkyl and —C₁-C₃ alkyl are substituted with hydroxyl and/or carboxyl.
   22. The Linker intermediate or Linker of embodiment 21, wherein the PEG unit is selected from the following, or a salt thereof:

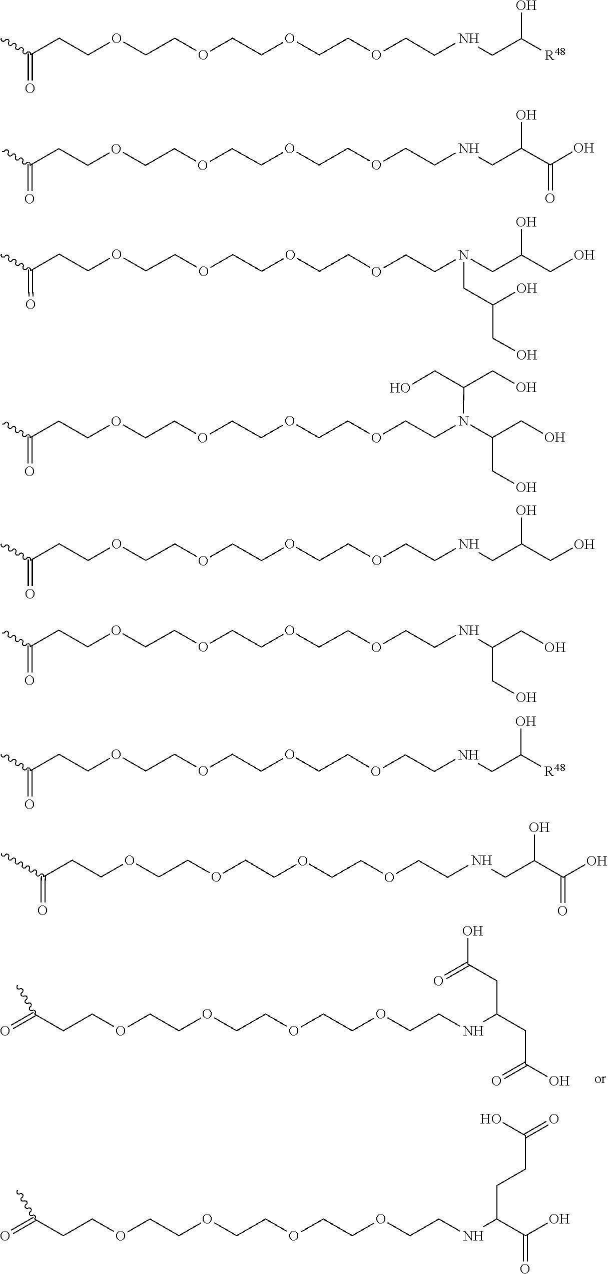

wherein R⁴⁸ is selected from H, OH, CH₂OH, COOH or —C₁-C₆ alkyl substituted with hydroxyl or carboxyl; and the wavy line at the left side indicates the attachment site to the subunit of the Amino Acid unit or the portion of the Linker subunit.
   23. The Linker intermediate or Linker of any of embodiments 5 to 6, wherein one of R²⁴ and R²⁵ is selected from H, substituted —C(O)—C₁-C₈ alkyl, substituted —C(O)—C₁-C₄, and substituted —C(O)—C₁-C₃ alkyl and the other of R²⁴ and R²⁵ is selected from substituted —C(O)—C₁-C₈ alkyl, substituted —C(O)—C₁-C₄ alkyl, substituted —C(O)—C₁-C₃ alkyl, substituted —C₁-C₈ alkyl, substituted —C₁-C₄ alkyl, and substituted —C₁-C₃ alkyl, wherein substituted —C(O)—C₁-C₈ alkyl, substituted —C(O)—C₁-C₄ alkyl, substituted —C(O)—C₁-C₃ alkyl, substituted —C₁-C₈ alkyl, —C₁-C₄ alkyl and —C₁-C₃ alkyl are substituted with hydroxyl and/or carboxyl.
   24. The Linker intermediate or Linker of embodiment 23, wherein the PEG unit is selected from the following, or a salt thereof:

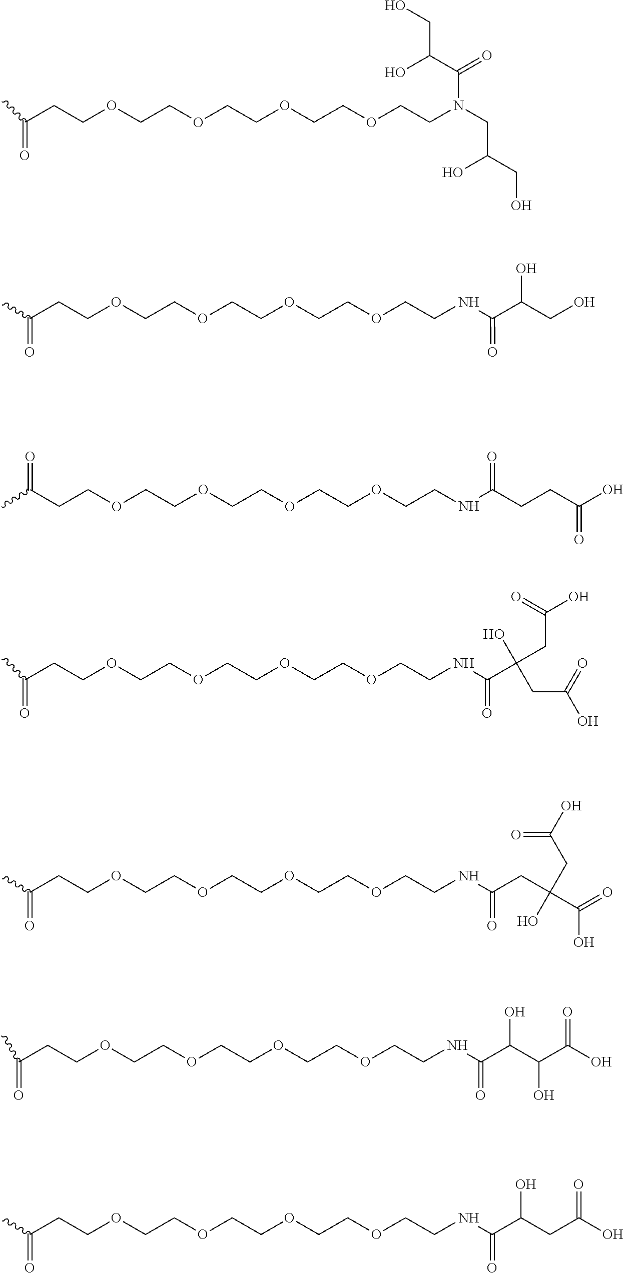

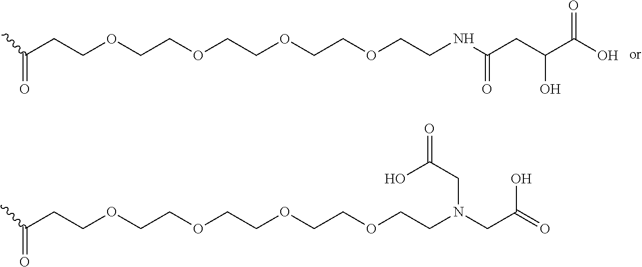

wherein the wavy line at the left side indicates the attachment site to the subunit of the Amino Acid unit or the portion of the Linker subunit.
   25. The Linker intermediate or Linker of any of embodiments 5 to 6, wherein R²⁴ and R²⁵ are selected from H and optionally substituted aryl; provided that both R²⁴ and R²⁵ are not H.
   26. The Linker intermediate or Linker of embodiment 25, wherein the PEG unit is selected from the following, or a salt thereof:

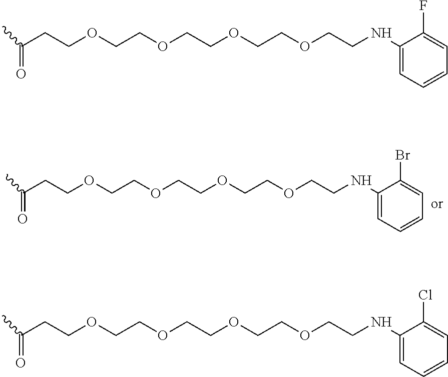

wherein the wavy line at the left side indicates the attachment site to the subunit of the Amino Acid unit or the portion of the Linker subunit.
   27. The Linker intermediate or Linker of any of embodiments 5 to 6, wherein R²⁴ and R²⁵ together form an optionally substituted C₃-C₈ heterocycle or heteroaryl.
   28. The Linker intermediate or Linker of embodiment 27, wherein the PEG unit is:

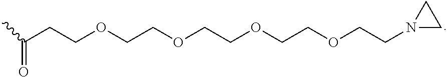

29. The Linker intermediate or Linker of any of embodiments 5 to 6, wherein R²⁴ and R²⁵ are independently selected from H and a chelator, wherein the chelator is optionally attached to the nitrogen of —NR²⁴R²⁵ by an alkylene, arylene, carbocyclo, heteroarylene or heterocarbocylo; provided that both R²⁴ and R²⁵ are not H.
30. The Linker intermediate or Linker of embodiment 29, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine.
31. The Linker Intermediate or Linker of embodiment 30, wherein the PEG unit is selected from the following, or a salt thereof:

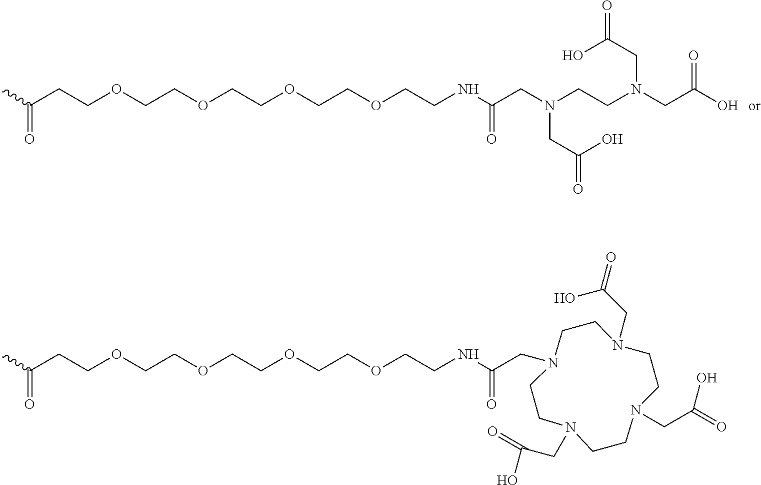

wherein the wavy line at the left side indicates the attachment site to the subunit of the Amino Acid unit or the portion of the Linker subunit.
   32. The Linker intermediate or Linker of any of embodiments 5 to 19, wherein each monosaccharide is independently selected from:
a C₅ or C₆ sugar selected from glucose, ribose, galactose, mannose, arabinose, 2-deoxyglucose, glyceraldehyde, erythrose, threose, xylose, lyxose, allose, altrose, gulose, idose talose, aldose, ketose, glucosamine, N-acetyl glucosamine, galactosamine, and N-acetyl galactosamine;
a sugar acid selected from gluconic acid, aldonic acid, uronic acid and ulosonic acid; or
an amino sugar is selected from glucosamine, N-acetyl glucosamine, galactosamine, and N-acetyl galactosamine.
   33. The Linker intermediate or Linker of any of embodiments 5 to 32, wherein R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof.
   34. The Linker intermediate or Linker of any of embodiments 1 to 4, wherein the PEG unit has the formula selected from the following:
(a)

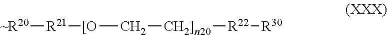

or a salt thereof, wherein:
   R²⁰ is a functional group for attachment to a subunit of the Amino Acid unit (if present) and/or a portion of Linker Subunit L2;
   R²¹ and R²² are each optional and, if present, are independently, C₁-C₃ alkylene groups;
   R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
   the wavy line (˜) indicates the attachment site to R²⁰; and n20 is 1 to 26;
(b)

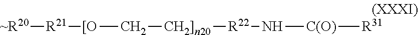

or a salt thereof, wherein:
   R²⁰ is a functional group for attachment to a subunit of the Amino Acid unit (if present) or a portion of the Linker Subunit L2;
   R²¹ and R²² are each, independently, optional C₁-C₃ alkylene groups;
   R³¹ is a branched polyethylene glycol chain, each branch having 1 to 26 ethylene glycol subunits and each branch having an R³⁵ at its terminus;
   R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
   the wavy line (˜) indicates the attachment site to R²⁰; and n20 is 1 to 26;
(c)

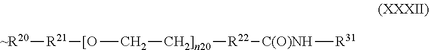

or a salt thereof, wherein:
   R²⁰ is a functional group for attachment to a subunit of the Amino Acid unit (if present) or a portion of the Linker Subunit L2;
   R²¹ and R²² are each optional and are, independently, C₁-C₃ alkylene groups;
   R³¹ is a branched polyethylene glycol chain, each branch, independently, having 1 to 26 ethylene glycol subunits and each branch having an R³⁵ at its terminus;
   R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl;
   the wavy line (˜) indicates the attachment site to R²⁰; and
   n20 is 1 to 26; and
(d)

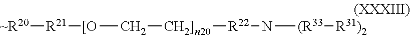

or a salt thereof, wherein:
   R²⁰ is a functional group for attachment to a subunit of the Amino Acid unit (if present) or a portion of the Linker Subunit L2;
   R²¹ and R²² are each optional and are C₁-C₃ alkylene groups;
   R³¹ is a branched polyethylene glycol chain, each branch having 1 to 26 ethylene glycol subunits and each branch having an R³⁵ at its terminus;
   R³³ is C₁-C₃ alkylene, C₁-C₃ alkylene-C(O), C(O)—C₁-C₃ alkylene, or —C(O)—C₁-C₃ alkylene-C(O);
   R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl;
   the wavy line (˜) indicates the attachment site to R²⁰; and n20 is 1 to 26.
      35. The Linker intermediate or Linker of any of embodiments 1 to 4, wherein the PEG unit has a formula selected from the following, or a salt thereof:

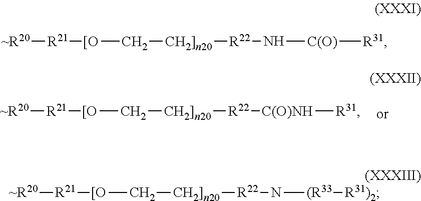

wherein R²⁰ is a functional group for attachment to a subunit of the Amino Acid unit (if present) or a portion of the Linker Subunit L2; R²¹ and R²² are each optional and are C₁-C₃ alkylene groups; R³¹ is a branched polyethylene glycol chain, each branch having 1 to 26 ethylene glycol subunits and each branch having an R³⁵ at its terminus; R³³ is C₁-C₃ alkylene, —C₁-C₃ alkylene-C(O), —C(O)—C₁-C₃ alkylene or —C(O)—C₁-C₃ alkylene-C(O); R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 1 to 26.
   36. The Linker intermediate or Linker of embodiment 35, wherein the PEG unit is selected from the following:

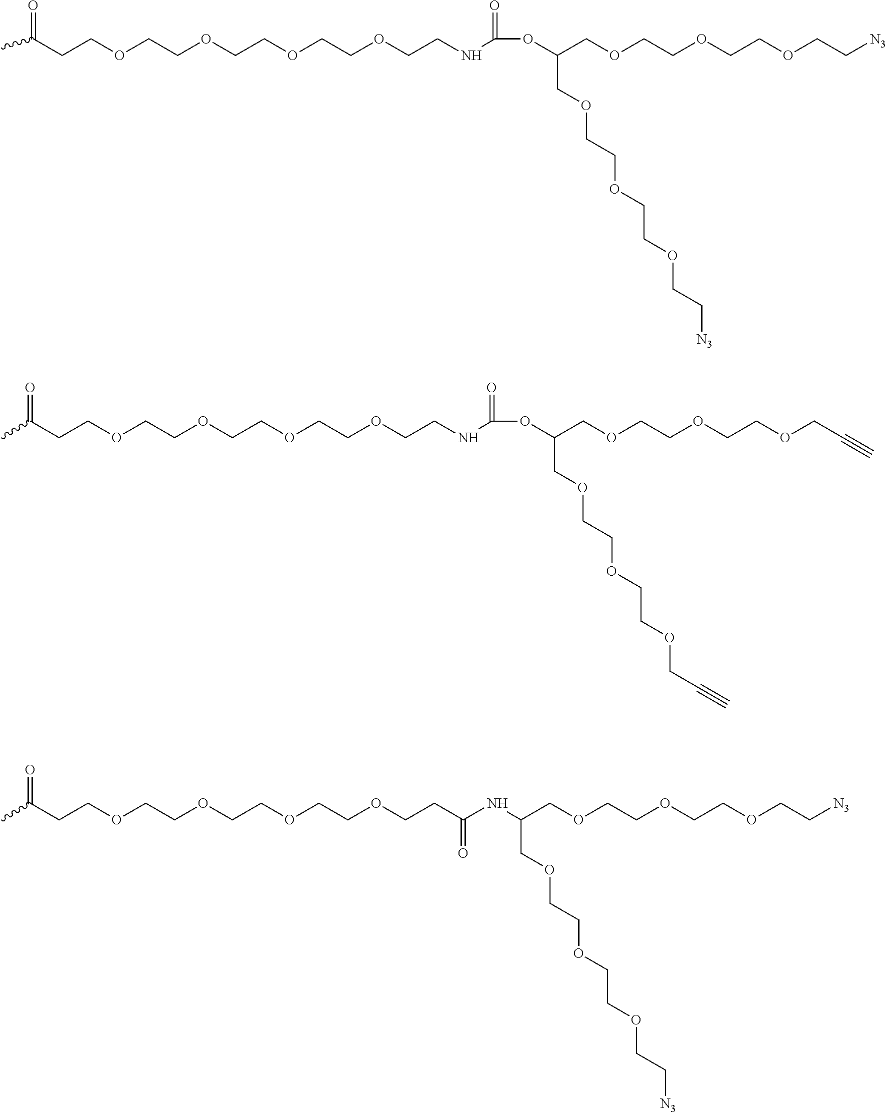

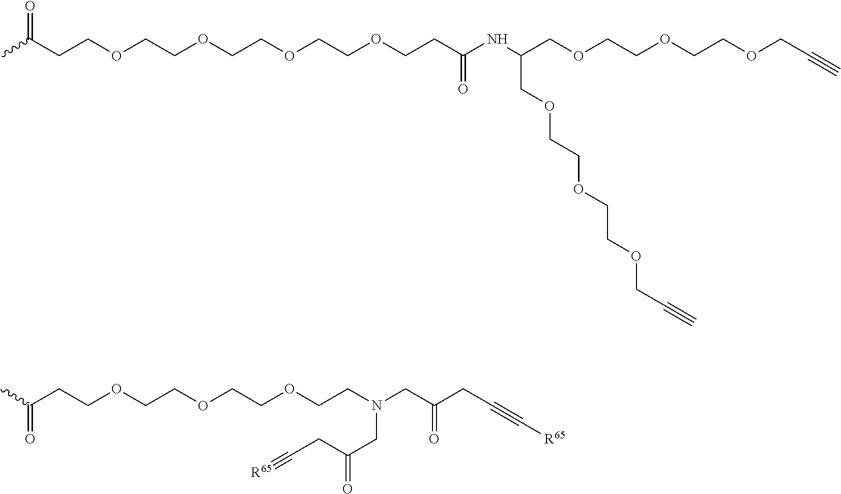

wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line at the left side indicates the attachment site to the subunit of the Amino Acid unit or the portion of the Linker subunit.
   37. The Linker intermediate or Linker of any of embodiments 34 to 37, wherein R²⁰ is selected from carboxyl, amino, alkynyl, azido, hydroxyl, carbonyl, carbamate, urea, thiocarbamate, thiourea, sulfonamide, acyl sulfonamide, alkyl sulfonate or protected forms thereof.
   38. The Linker intermediate or Linker of any of embodiments 1 to 4, wherein the Carboxyl unit has the following formula:

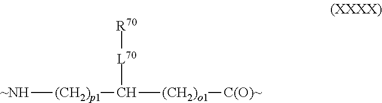

or a salt thereof, wherein:
(a)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
R⁷⁰ is —NR⁷¹(R⁷²-R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is absent or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide;
   each wavy line (˜) indicates an attachment site for another subunit of an Amino Acid unit (AA), the Linker subunit L2, or the Stretcher unit (L1); and each of p1 and o1 are independently selected from 0 to 2;
   or
(b)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is —NR⁷¹(R⁷⁵_(R⁷³)2), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; each wavy line (˜) indicates an attachment site for another subunit of an Amino Acid unit (AA), the Linker subunit L2, or the Stretcher unit (L1); and
   each of p1 and 01 are independently selected from 0 to 2;
   or
(c)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—;
   R⁷⁰ is —N(R⁷⁴-R⁷³)(R⁷2-R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; each wavy line (˜) indicates an attachment site for another subunit of an Amino Acid unit (AA), the Linker subunit L2, or the Stretcher unit (L1); and each of p1 and 01 are independently selected from 0 to 2.
      39. The Linker intermediate or Linker of any of embodiments 1 to 38, comprising at least one Sugar unit.
      40. The Linker intermediate or Linker of any of embodiments 1 to 38, comprising at least one PEG unit.
      41. The Linker intermediate or Linker of any of embodiments 1 to 38, comprising at least one Carboxyl unit.
      42. The Linker intermediate or Linker of any of embodiments 1 to 38, comprising at least two Polar units, each Polar unit selected from a Sugar unit, a PEG unit and a Carboxyl unit.
      43. The Linker intermediate or Linker of any of embodiments 1 to 38, comprising at least one Sugar unit and a PEG unit or a Carboxyl unit.
      44. The Linker intermediate or Linker of any of embodiments 1 to 38, comprising at least one Carboxyl unit and a PEG unit.
      45. The Linker intermediate or Linker of any of embodiments 1 to 44, wherein the Amino Acid unit (AA) is present (s=1).
      46. The Linker intermediate or Linker of any of embodiments 1 to 45, wherein the Amino Acid unit comprises at least one Polar unit.
      47. The Linker intermediate or Linker of any of embodiments 1 to 45, wherein ˜AA-L2˜ has a formula selected from the following:

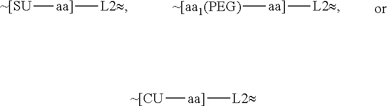

wherein the square brackets indicate the Amino Acid unit, each aa is an optional subunit of AA, L2 is the Linker Subunit, each wavy line (˜) indicates an attachment site for a Stretcher unit; aa₁(PEG) is a PEG unit attached to an amino acid subunit of AA, SU is a Sugar unit attached to a subunit of AA or to L2, and CU is a Carboxyl unit attached to a subunit of AA or to L2; and the double wavy (≈) line indicates an attachment site for a Drug unit, wherein aa and aa₁ are independently selected from alpha, beta and gamma amino acids and derivatives thereof.
   48. The Linker intermediate or Linker of any of embodiments 1 to 45, wherein ˜AA-L2˜ has a formula selected from the following:

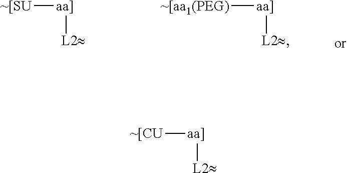

wherein the square brackets indicate the Amino Acid unit, each aa is an amino acid subunit of AA, L2 is the Linker Subunit attached to a side chain of aa, the wavy line (˜) indicates an attachment site for a Stretcher unit; aa₁(PEG) is a PEG unit attached to aa, SU is a Sugar unit attached to aa, CU is a Carboxyl unit attached to aa, and the double wavy (≈) line indicates an attachment site for a Drug unit; wherein aa and aa, are independently selected from alpha, beta and gamma amino acids and derivatives thereof.
   49. The Linker intermediate or Linker of any of embodiments 1 to 46, wherein the Amino Acid unit comprises at least two Polar units.
   50. The Linker intermediate or Linker of embodiment 49, wherein ˜AA-L2 ˜ has a formula selected from the following:

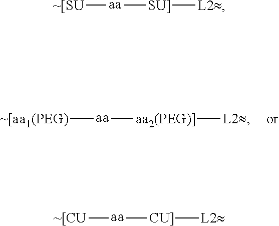

wherein the square brackets indicate the Amino Acid unit, aa is an optional subunit of AA, L2 is the Linker Subunit, the wavy line (˜) indicates an attachment site for a Stretcher unit; each of aa₁(PEG) and aa₂(PEG) is a PEG unit attached to aa or to the other PEG unit; each SU is a Sugar unit attached to aa or the other Sugar unit, each CU is a Carboxyl unit attached to aa or to the other Carboxyl unit, and the double wavy (≈) line indicates an attachment site for a Drug unit; wherein aa, aa, and aa₂ are independently selected from selected from alpha, beta and gamma amino acids and derivatives thereof.
   51. The Linker intermediate or Linker of embodiment 49, wherein ˜AA-L2˜ has a formula selected from the following:

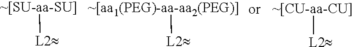

wherein the square brackets indicate the Amino Acid unit, aa is an amino acid subunit of AA, L2 is a Linker Subunit attached to a side chain of aa, each wavy line (˜) indicates an attachment site for a Stretcher unit; each of aa₁(PEG) and aa₂(PEG) is a PEG unit attached to aa, each SU is a Sugar unit attached to aa; each CU is a Carboxyl unit attached to aa; and the double wavy (≈) line indicates an attachment site for a Drug unit; wherein each of aa, aa₁ and aa₂ is independently selected from alpha, beta and gamma amino acids and derivatives thereof.
   52. The Linker intermediate or Linker of any of the previous embodiments, wherein Linker Subunit L2 is a cleavable linker unit.
   53. The Linker intermediate or Linker of embodiment 52, wherein Linker Subunit L2 comprises a peptide that is cleavable by an intracellular protease.
   54. The Linker intermediate or Linker of embodiment 53, wherein the cleavable peptide comprises a valine-citrulline peptide, a valine-alanine peptide, a valine-lysine peptide, a phenylalanine-lysine peptide, or a glycine-glycine-phenylalanine-glycine peptide (SEQ ID NO: 43).
   55. The Linker intermediate or Linker of any of the previous embodiments, wherein Linker Subunit L2 comprises at least one Polar unit.
   56. The Linker intermediate or Linker of any of the previous embodiments, wherein the Polar unit is a Sugar unit (SU).
   57. The Linker intermediate or Linker of embodiment 56, wherein the cleavable peptide comprises a SU-valine-citrulline peptide, a SU-valine-lysine peptide, a SU-valine-alanine peptide, a SU-phenylalanine-lysine peptide, or a SU-glycine-glycine-phenylalanine-glycine peptide (SEQ ID NO: 44).
   58. The Linker intermediate or Linker of embodiment 55, wherein the Polar unit is a Carboxyl unit (CU).
   59. The Linker intermediate or Linker of embodiment 58, wherein the cleavable peptide comprises a CU-valine-citrulline peptide, a CU-valine-lysine peptide, a valine-(CU-lysine) peptide, a CU-valine-alanine peptide, a CU-phenylalanine-lysine peptide, a phenylalanine-(CU-lysine) peptide or a CU-glycine-glycine-phenylalanine-glycine peptide (SEQ ID NO: 45), wherein CU-lysine is a Carboxylate unit comprising a lysine residue.
   60. The Linker intermediate or Linker of embodiment 55, wherein the Polar unit is a PEG unit (PEG).
   61. The Linker intermediate or Linker of embodiment 60, wherein the cleavable peptide comprises a Lys(PEG)-valine-citrulline peptide, a valine-Cit(PEG) peptide, a Lys(PEG)-valine-lysine peptide, a valine-lysine(PEG) peptide, a Lys(PEG)-valine-alanine peptide, a Lys(PEG)-phenylalanine-lysine peptide, a phenylalanine-Lys(PEG)) peptide or a Lys(PEG)-glycine-glycine-phenylalanine-glycine peptide (SEQ ID NO: 46), wherein Lys(PEG) and Cit(PEG) comprise a PEG unit attached to a lysine residue or a citrulline residue, respectively.
   62. The Linker intermediate or Linker of any of embodiments 52 to 61, wherein the cleavable peptide is attached to para-aminobenzyl alcohol self immolative group (PABA).
   63. The Linker intermediate or Linker of embodiment 62, wherein ˜AA-L2˜ has one of the following structures:

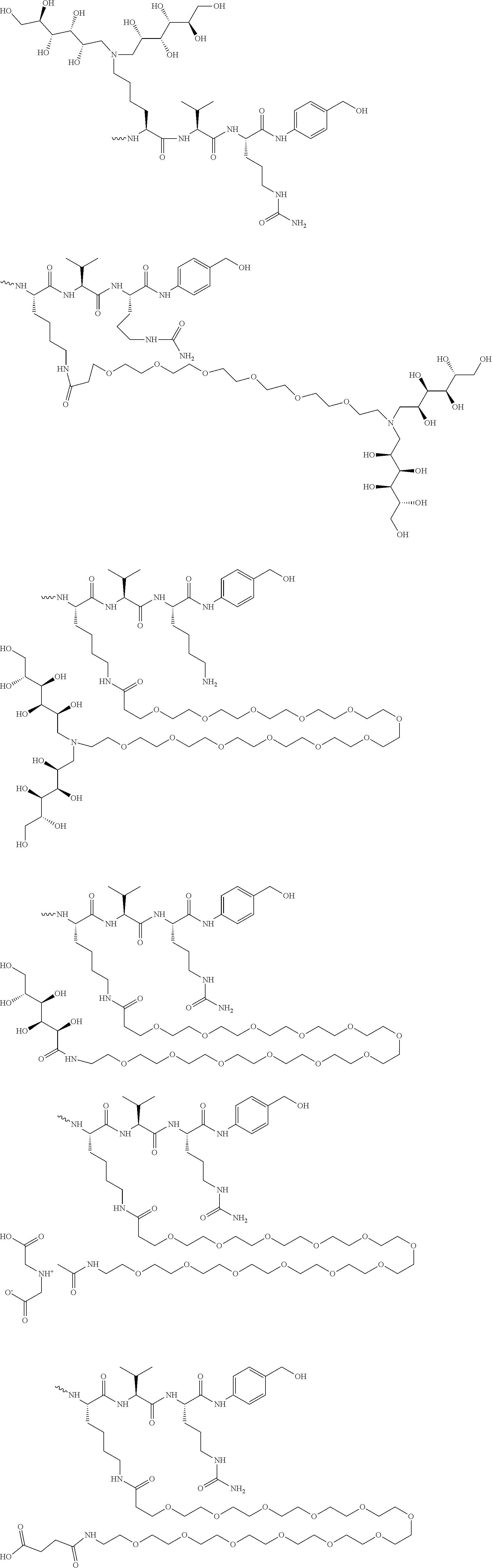

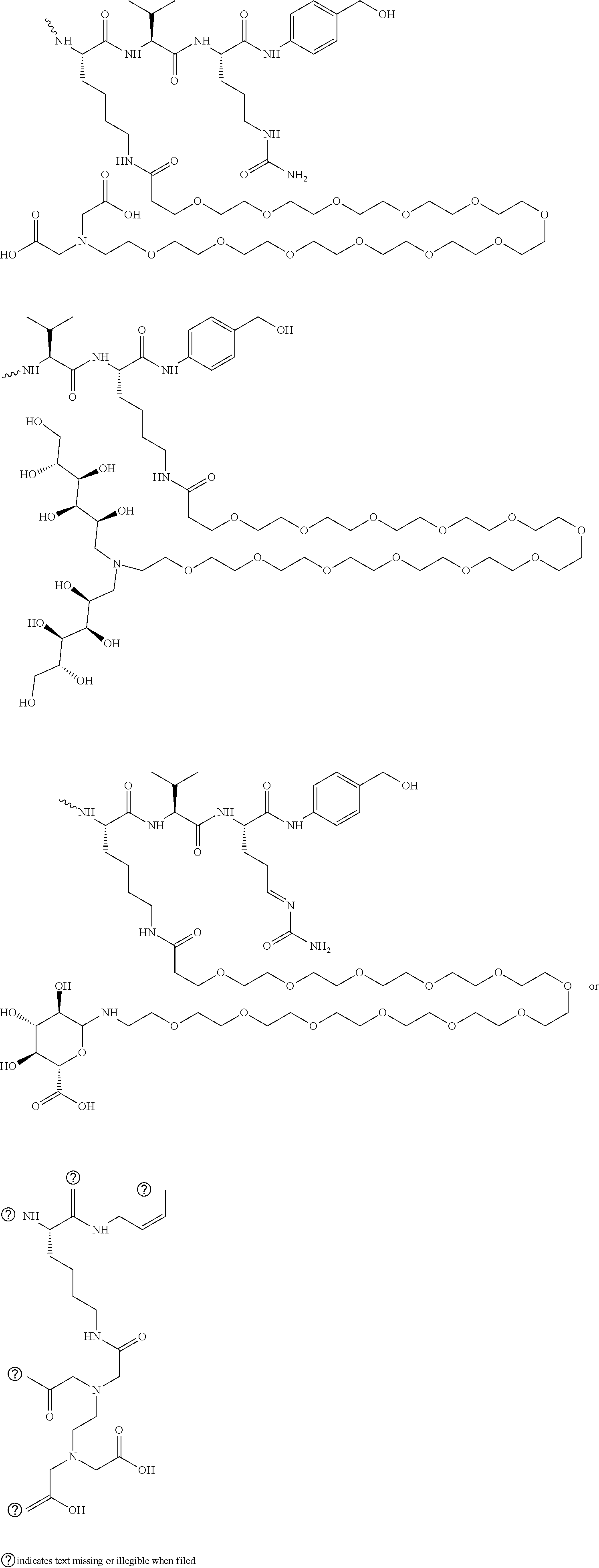

wherein the wavy line on the amino group indicates an attachment site for a Stretcher unit.
   64. The Linker intermediate or Linker of any of embodiments 52 to 62, wherein L2 is attached to a side chain of a subunit of AA.
   65. The Linker intermediate or Linker of embodiment 64, wherein ˜AA-L2˜ has one of the following structures:

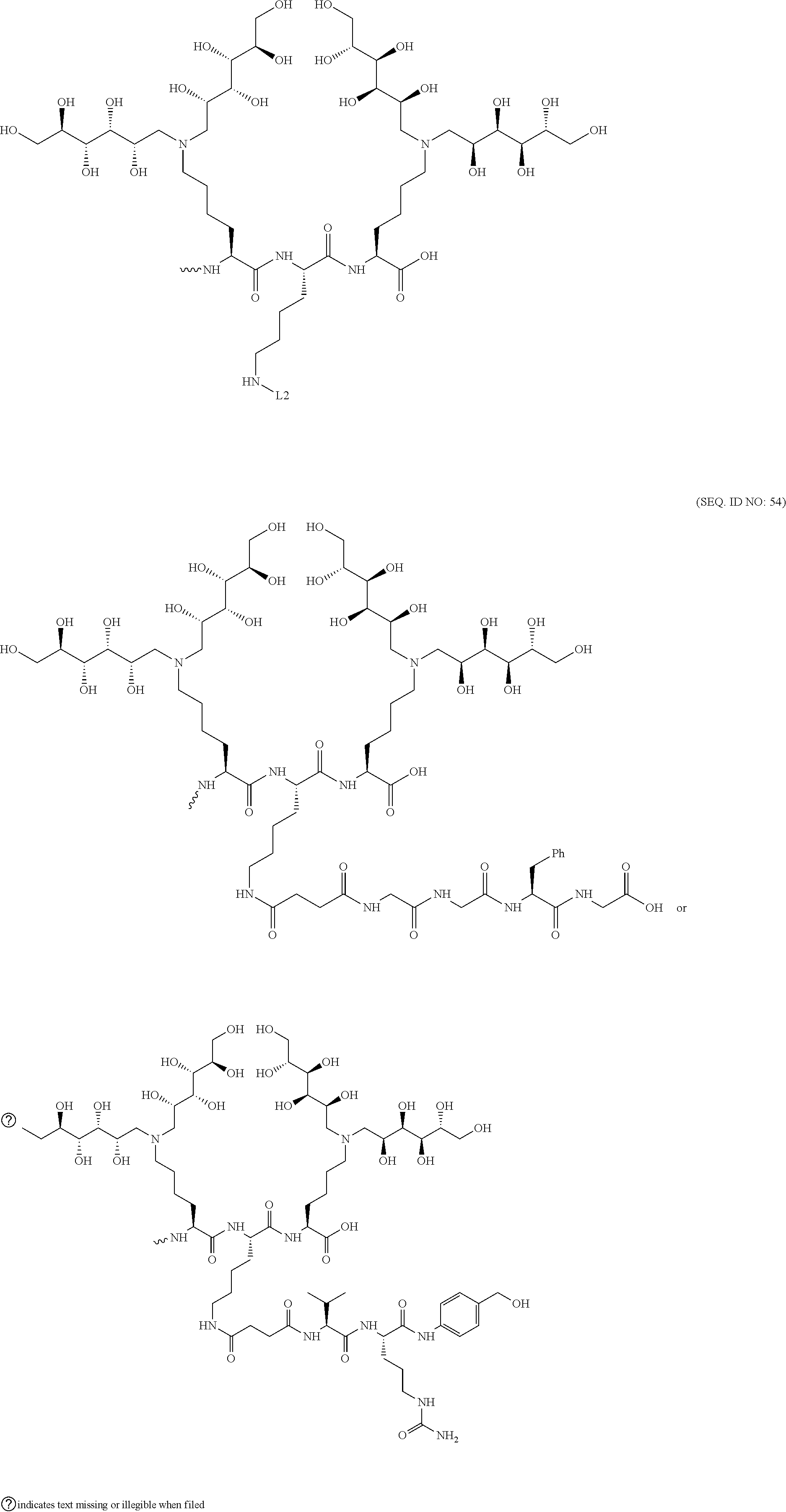

wherein the wavy line on the amino group indicates an attachment site for a Stretcher unit.
   66. The Linker intermediate or Linker of any of the embodiments 1 to 62, wherein the Amino Acid unit is joined to Linker Subunit L2 by a non-peptidic linking group.
   67. The linker of embodiment 66, wherein the non-peptidic linking group is selected from C₁-C₁₀ alkylene, C₂-C₁₀ alkenylene, C₂-C₁₀ alkynylene, or polyethylene glycol.
   68. The Linker intermediate of any of the previous embodiments, further comprising a Stretcher unit.
   69. The Linker of embodiment 68, wherein the Stretcher unit is selected from the following:

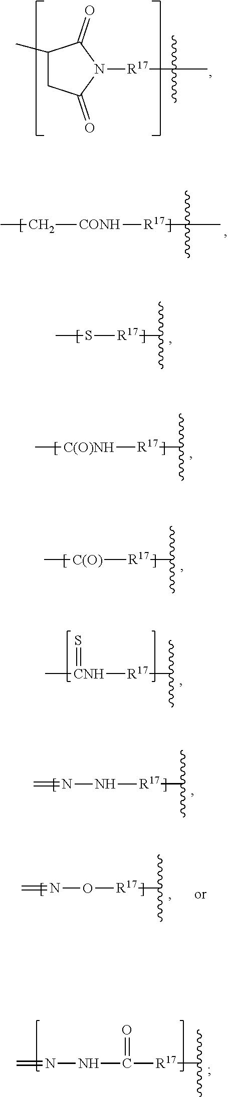

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene-(where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, -C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)—NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)—NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the Stretcher unit comprises maleimido(C₁-C₁₀alkylene-C(O)—, maleimido(CH₂OCH₂)ₚ₂(C₁-C₁₀alkyene)C(O)—, maleimido(C₁-C₁₀alkyene)(CH₂OCH₂)ₚ₂C(O)—, or a ring open form thereof, wherein p2 is from 1 to 26.
   70. The Linker of embodiment 69, having one of the following structures:

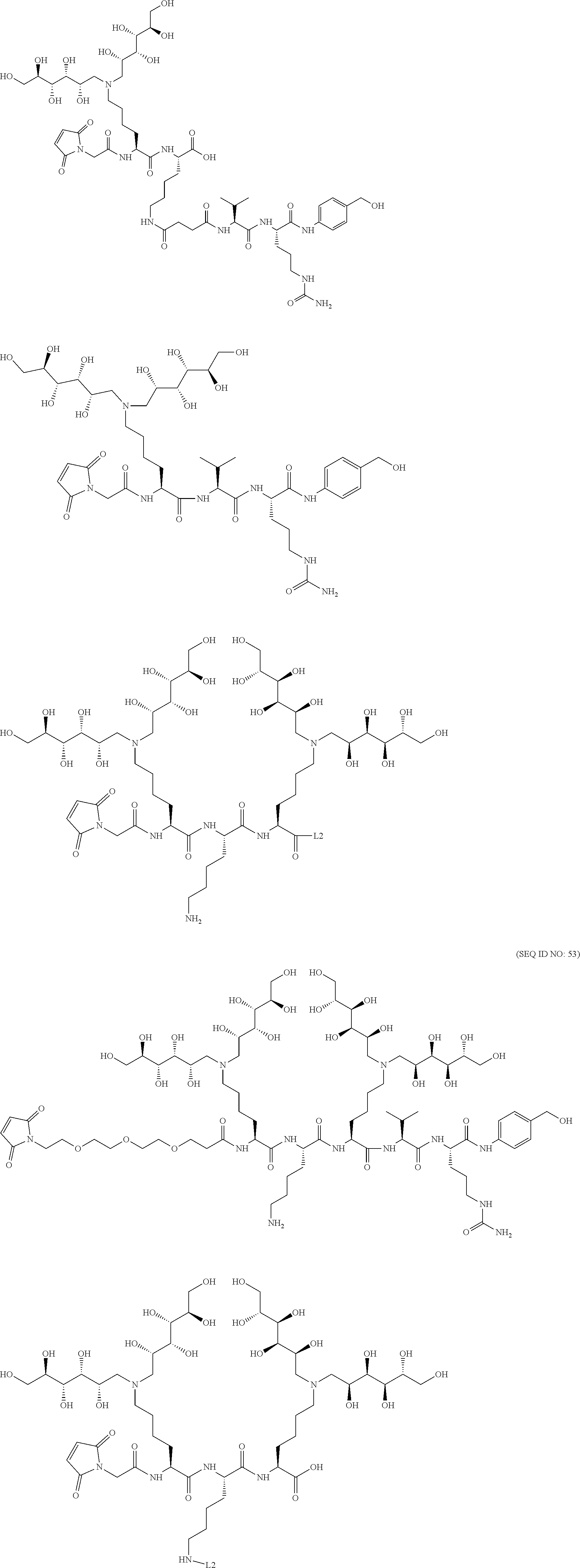

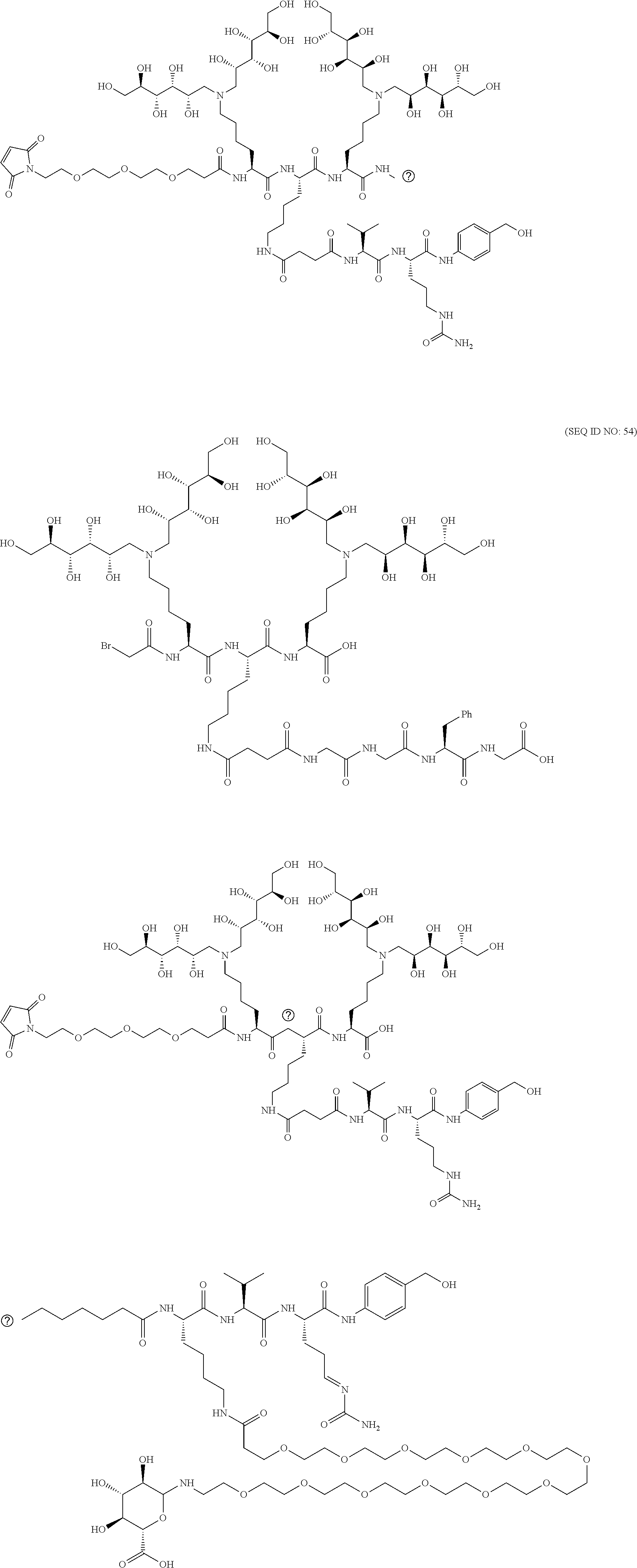

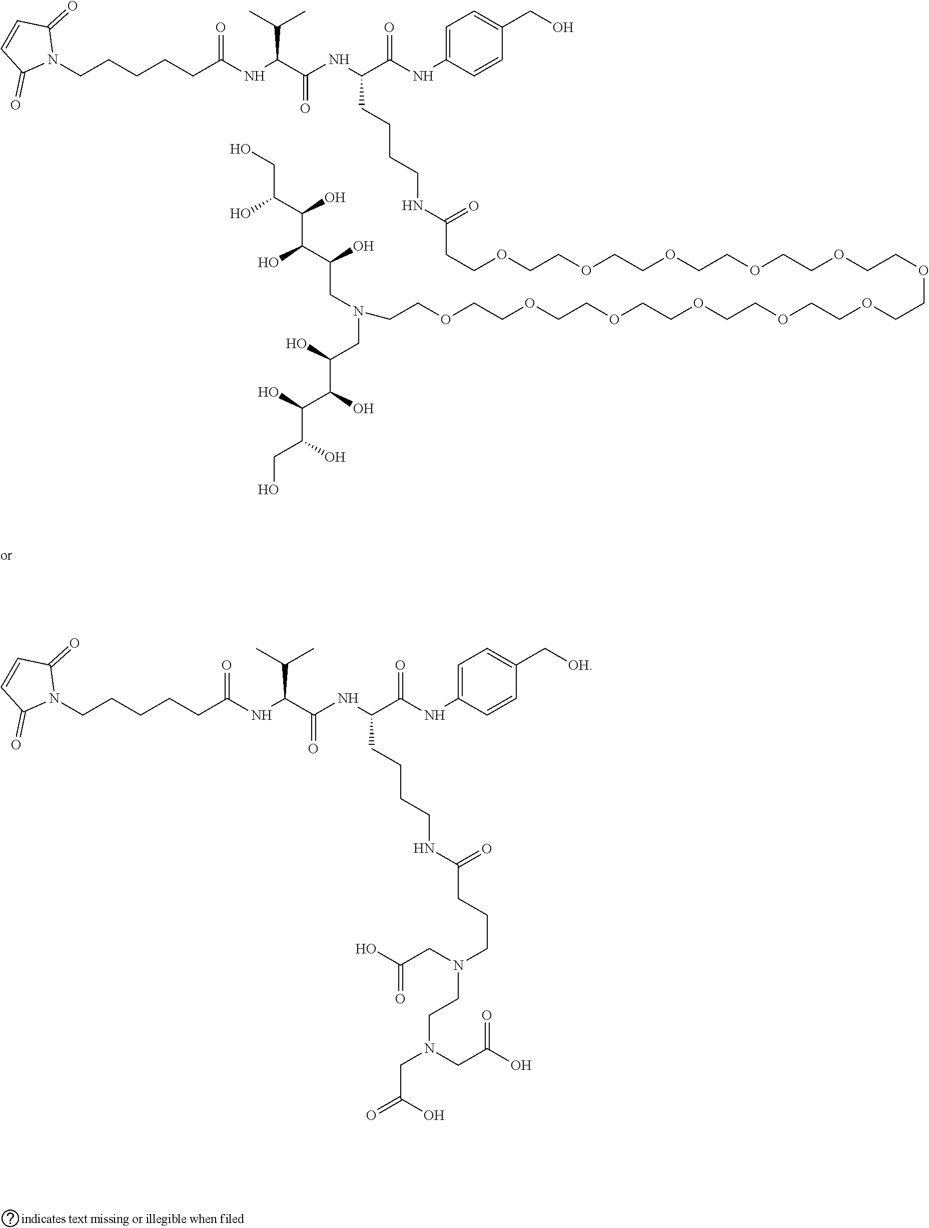 [SMILES: CC(C)[C@H](NC(=O)CCCCCN1C(=O)C=CC1=O)C(=O)N[C@@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)Nc1ccc(CO)cc1 ; CC(C)[C@H](NC(=O)CCCCCN1C(=O)C=CC1=O)C(=O)N[C@@H](CCCCNC(=O)CCCN(CCN(CC(=O)O)CC(=O)O)CC(=O)O)C(=O)Nc1ccc(CO)cc1]

71. The Linker of any of the previous embodiments, further comprising at least one Drug unit attached to Linker Subunit L2 to form a Drug-Linker.
72. The Drug-Linker of embodiment 71, wherein the Drug unit is selected from a cytotoxic agent, an immune modulatory agent, a nucleic acid, a growth inhibitory agent, a PROTAC, a toxin, a radioactive isotope and a chelating ligand.
73. The Drug-Linker of embodiment 72, wherein the Drug unit is a cytotoxic agent.
74. The Drug-Linker of embodiment 73, wherein the cytotoxic agent is selected from the group consisting of an auristatin, a maytansinoid, a camptothecin, a duocarmycin, and a calicheamicin.
75. The Drug-Linker of embodiment 74, wherein the cytotoxic agent is an auristatin.
76. The Drug-Linker of embodiment 75, wherein the cytotoxic agent is MMAE or MMAF.
77. The Drug-Linker of embodiment 74, wherein the cytotoxic agent is a camptothecin.
78. The Drug-Linker of embodiment 77, wherein the cytotoxic agent is exatecan or SN-38.
79. The Drug-Linker of embodiment 73, wherein the cytotoxic agent is a calicheamicin.
80. The Drug-Linker of embodiment 73, wherein the cytotoxic agent is a maytansinoid.
81. The Drug-Linker of embodiment 80, wherein the maytansinoid is maytansine, maytansinol or ansamatocin-2.
82. The Drug-Linker of embodiment 72, wherein the Drug unit is an immune modulatory agent.
83. The Drug-Linker of embodiment 82, wherein the immune modulatory agent is selected from a TRL7 agonist, a TLR8 agonist, a STING agonist, or a RIG-1 agonist.
84. The Drug-Linker of embodiment 83, wherein the immune modulatory agent is an TLR7 agonist.
85. The Drug-Linker of embodiment 84, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.
86. The Drug-Linker of embodiment 83, wherein the immune modulatory agent is a TLR8 agonist.
87. The Drug-Linker of embodiment 86, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.
88. The Drug-Linker of embodiment 83, wherein the immune modulatory agent is a STING agonist.
89. The Drug-Linker of embodiment 83, wherein the immune modulatory agent is a RIG-1 agonist.
90. The Drug-Linker of embodiment 89, wherein the RIG-1 agonist is selected from KIN1148, SB-9200, KIN700, KIN600, KIN500, KIN100, KIN101, KIN400 and KIN2000.
91. The Drug-Linker of embodiment 72, wherein the Drug unit is a chelating ligand.
92. The Drug-Linker of embodiment 91, wherein the chelating ligand is selected from platinum (Pt), ruthenium (Ru), rhodium (Rh), gold (Au), silver (Ag), copper (Cu), molybdenum (Mo), titanium (Ti), or iridum (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.
93. A conjugate comprising a Targeting unit attached to the Drug-linker of any of embodiments 72 to 92.
94. The conjugate of embodiment 93, wherein the Targeting unit is selected from an antibody or an antigen-binding portion thereof.
95. The conjugate of embodiment 94, wherein the Targeting unit is a monoclonal antibody, a Fab, a Fab′, an F(ab′), an Fv, a disulfide linked Fc, a scFv, a single domain antibody, a diabody, a bi-specific antibody, or a multi-specific antibody.
96. The conjugate of embodiment 93, wherein the Targeting unit is a diabody, a DART, an anticalin, an affibody, an avimer, a DARPin, or an adnectin.
97. The conjugate of any of embodiments 93 to 96, wherein the Targeting unit is mono-specific.
98. The conjugate of any of embodiments 93 to 97, wherein the Targeting unit is bivalent.
99. The conjugate of any of embodiments 93 to 96, wherein the Targeting unit is bispecific.
100. The conjugate of any of embodiments 93 to 99, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.
101. The conjugate of any of embodiments 93-100, selected from the following:

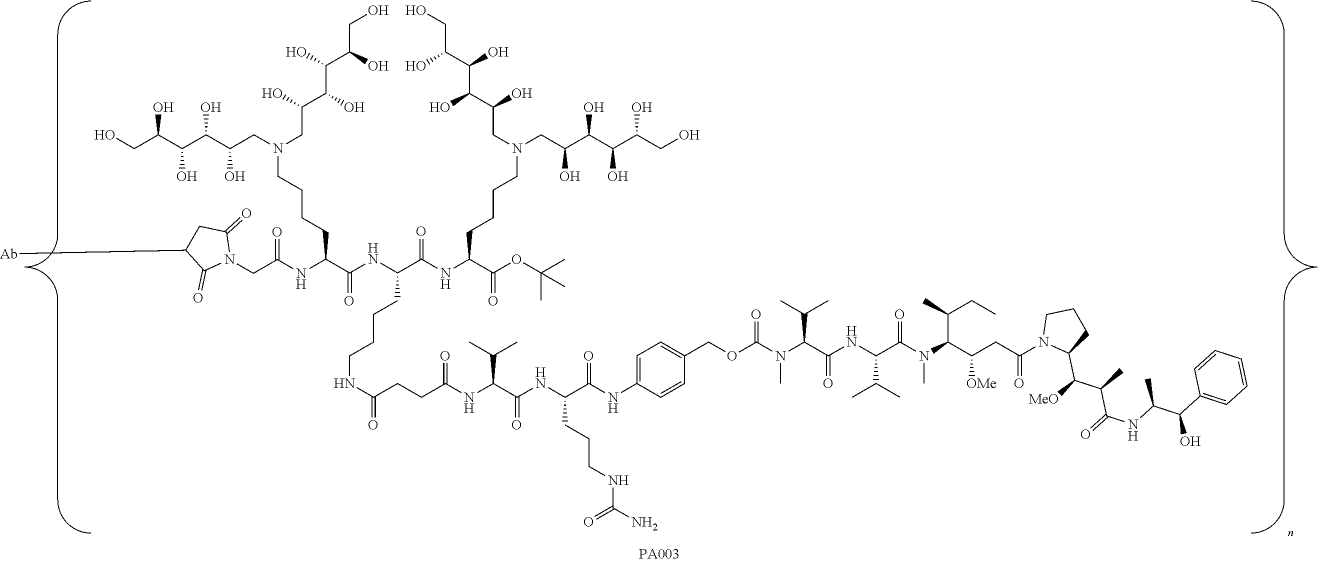

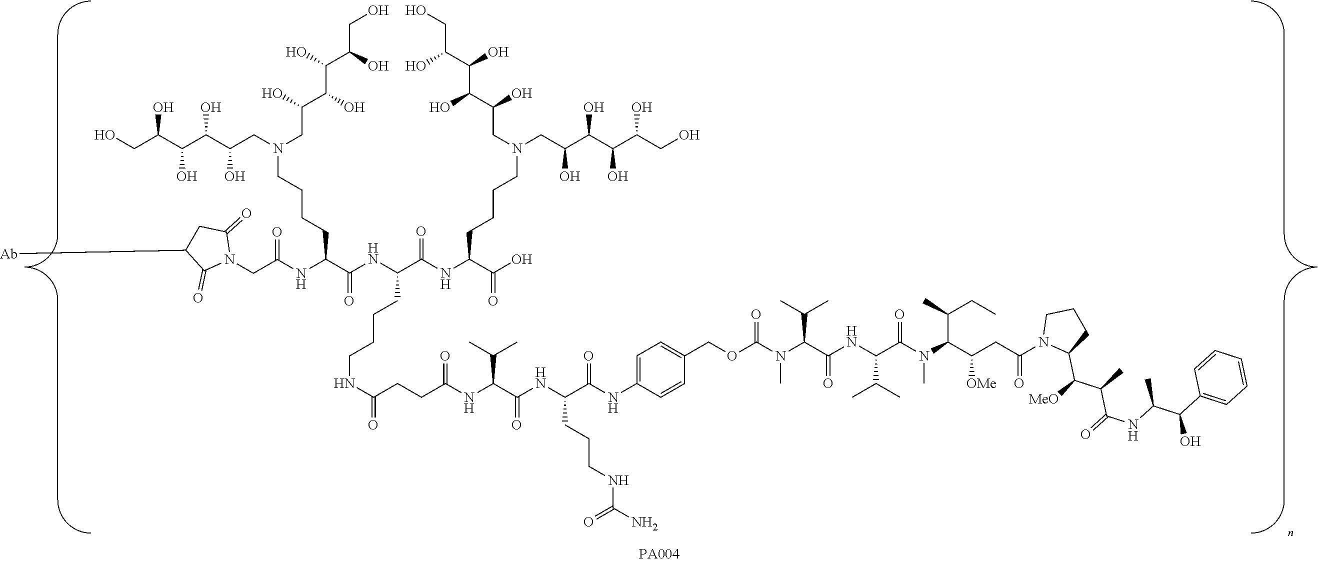

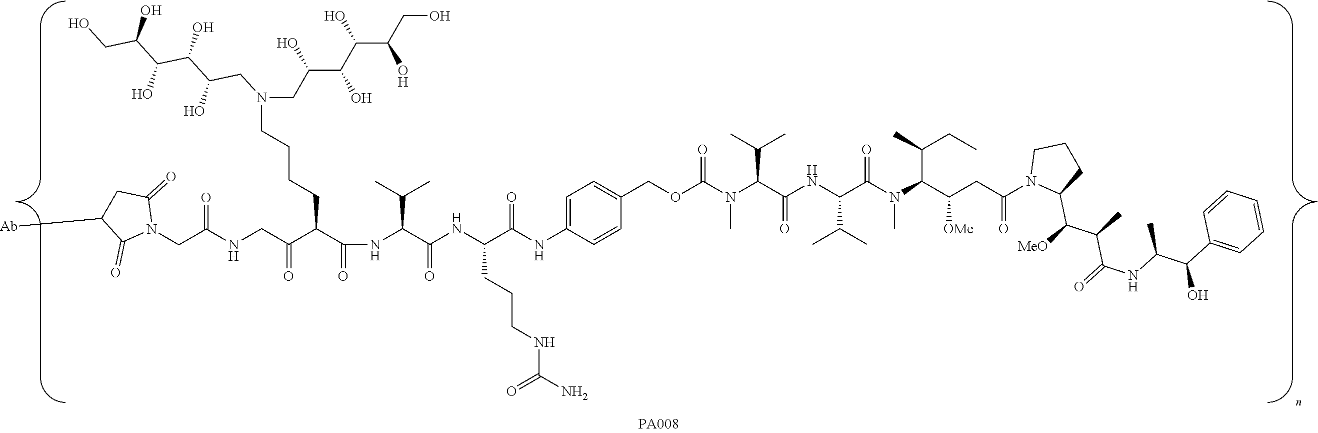

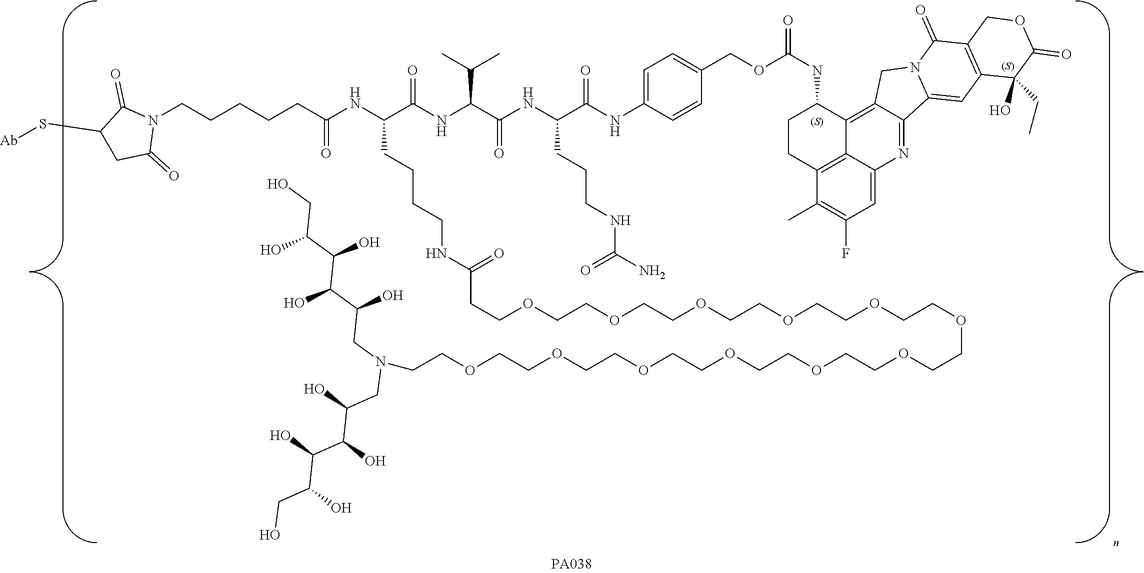

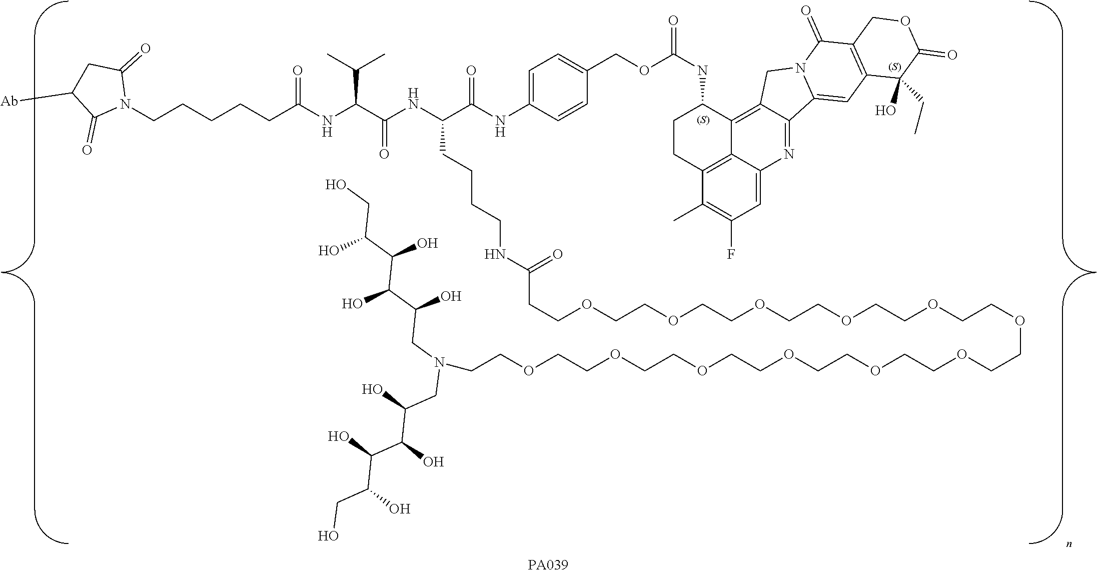

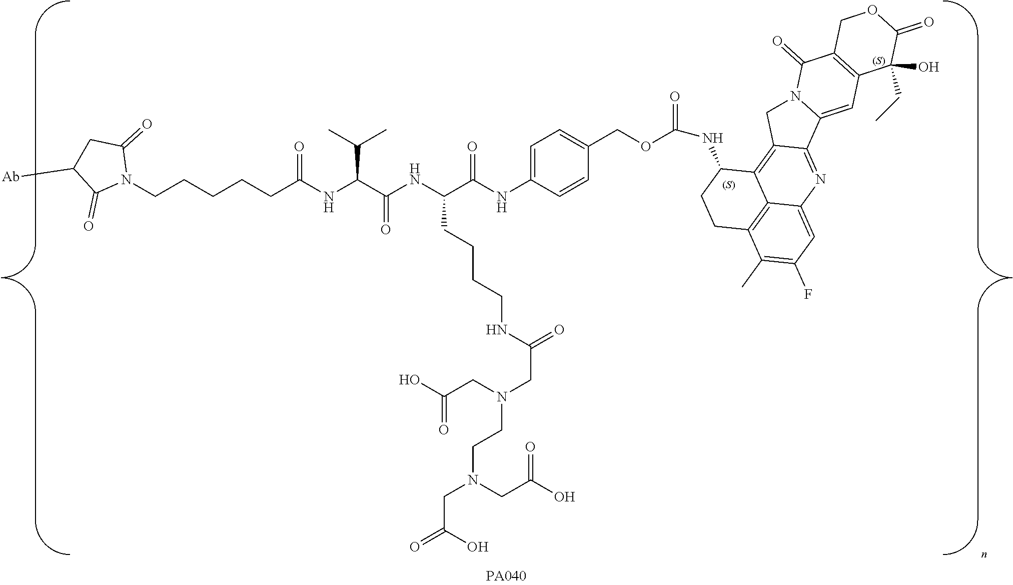

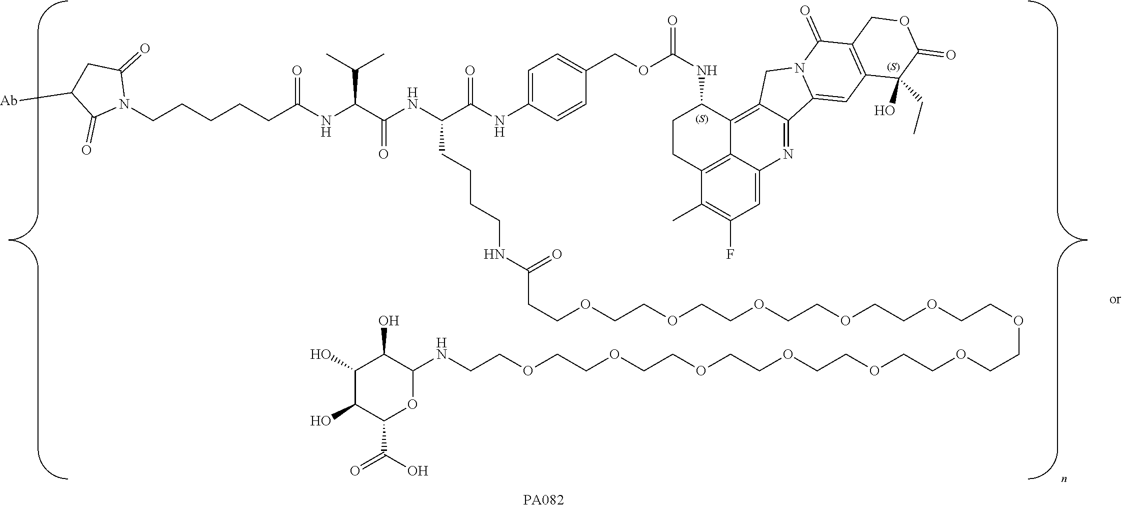

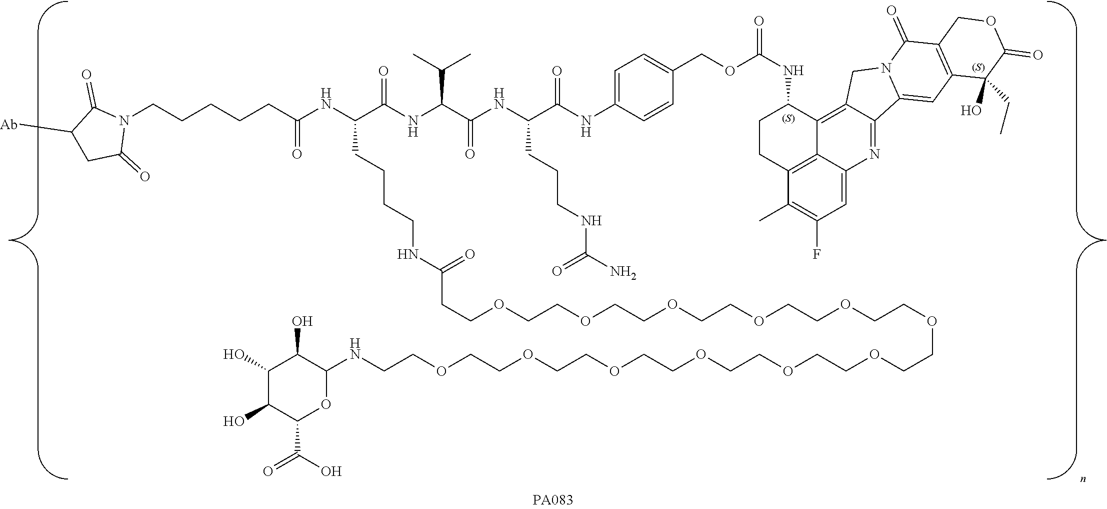

wherein Ab is a Targeting unit and n is pload.
   102. A pharmaceutical composition comprising the conjugate of any of embodiments 93 to 101 and a pharmaceutically acceptable carrier.
   103. A method of treating a subject in need thereof, comprising administering to the subject a conjugate of any of embodiments 93 to 101 or the pharmaceutical composition of embodiment 102, wherein the subject has cancer or an autoimmune disease and the conjugate binds to a target antigen associated with the cancer or autoimmune disease.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### General Methods

¹H NMR and other NMR spectra were recorded on Bruker AVIII 400 or Bruker AVIII 500. The data were processed with Nuts software or MestReNova software, measuring proton shifts in parts per million (ppm) downfield from an internal standard tetramethyl silane.

HPLC-MS measurement was run on Agilent 1200 HPLC/6100 SQ System using the following conditions:
Method A: Mobile Phase: A: Water (0.01% TFA) B: acetonitrile (0.01% TFA); Gradient Phase: 5% of B increasing to 95% of B in 15 min; Flow Rate: 1.0 mL/min; Column: XBridge C18, 4.6*150 mm, 3.5 um; Column Temperature: 40° C. Detectors: ADC ELSD, DAD (214 nm and 254 nm), ES-API.
Method B: Mobile Phase: A: Water (0.01% TFA) B: acetonitrile (0.01% TFA); Gradient Phase: 5% of B increasing to 95% of B in 15 min; Flow Rate: 1.0 mL/min; Column: SunFire C18, 4.6*150 mm, 3.5 μm; Column Temperature: 45° C. Detectors: ADC ELSD, DAD (214 nm and 254 nm), ES-API.
Method C: Mobile Phase: A: Water (10 mM NH₄HCO₃) B: acetonitrile; Gradient Phase: 5% to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), MSD (ES-API).

LCMS measurement was run on Agilent 1200 HPLC/6100 SQ System using the following conditions:
Method A: Mobile Phase: A: Water (0.01% TFA) B: acetonitrile (0.01% TFA); Gradient Phase: 5% of B increasing to 95% of B in 3 min; Flow Rate: 1.8-2.3 mL/min; Column: SunFire C18, 4.6*50 mm, 3.5 μm; Column Temperature: 50° C. Detectors: ADC ELSD, DAD (214 nm and 254 nm), ES-API.
Method B: Mobile Phase: A: Water (10 mM NH₄HCO₃) B: Acetonitrile; Gradient Phase: 5% to 95% of B in 3 min; Flow Rate: 1.8-2.3 mL/min; Column: XBridge C18, 4.6*50 mm, 3.5 μm; Column Temperature: 50° C. Detectors: ADC ELSD, DAD (214 nm and 254 nm), MSD (ES-API).

Preparative high pressure liquid chromatography (Prep-HPLC) was run on Gilson 281 using the following conditions:
Method A: Waters SunFire 10 μm C18 column (100 Å, 250×19 mm). Solvent A was water/0.01% trifluoroacetic acid (TFA) and solvent B was acetonitrile. The elution condition was a linear gradient increase of solvent B from 5% to 100% over a time period of 20 minutes at a flow rate of 30 mL/min.
Method B: Waters SunFire 10 μm C18 column (100 Å, 250×19 mm). Solvent A was water/0.05% formic acid (FA) and solvent B was acetonitrile. The elution condition was a linear gradient increase of solvent B from 5% to 100% over a time period of 20 minutes at a flow rate of 30 mL/min.
Method C: Waters Xbridge 10 μm C18 column (100 Å, 250×19 mm). Solvent A was water/10 mM ammonium bicarbonate (NH₄HCO₃) and solvent B was acetonitrile. The elution condition was a linear gradient increase of solvent B from 5% to 100% over a time period of 20 minutes at a flow rate of 30 mL/min.

Flash chromatography was performed on instrument of Biotage, with Agela Flash Column silica-CS; Reverse phase flash chromatography was performed on instrument of Biotage, with Boston ODS or Agela C₁₈.

### Example 1: Preparation of a Sugar Unit

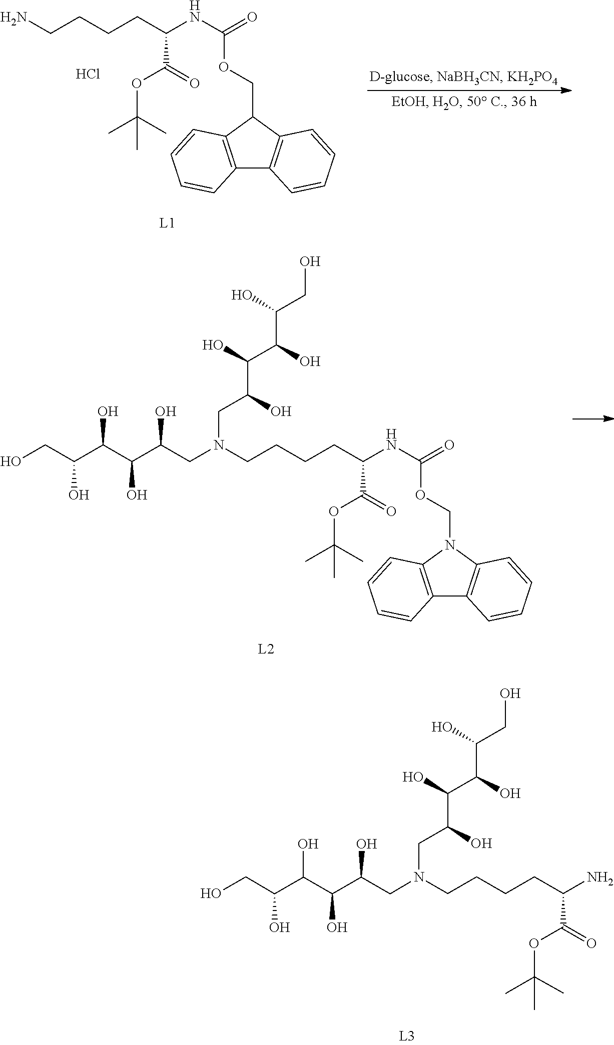 [SMILES: CC(C)(C)OC(=O)[C@H](CCCCN)NC(=O)OCC1c2ccccc2-c2ccccc21 ; Cl ; CC(C)(C)OC(=O)[C@H](CCCCN(C[C@H](O)[C@@H](O)[C@H](O)[C@H](O)CO)C[C@H](O)[C@@H](O)[C@H](O)[C@H](O)CO)NC(=O)OCn1c2ccccc2c2ccccc21 ; CC(C)(C)OC(=O)[C@@H](N)CCCCN(C[C@H](O)[C@@H](O)[C@H](O)[C@H](O)CO)C[C@H](O)[C@@H](O)C(O)[C@H](O)CO]

A Sugar unit was prepared as follows:

Step 1 A reaction mixture of compound L1 (5 g, 10.846 mmol), D-glucose (19.54 g, 108.460 mmol), NaBH₃CN (5.45 g, 86.768 mmol) and potassium dihydrogen phosphate (0.379 mL, 6.508 mmol) in water (40 mL) and ethanol (65 mL) was stirred at 50° C. under N₂ for 36 hr, until the reaction was complete as indicated by LCMS. The solvents were evaporated, and the residue was purified by C18 reversed-phase chromatography to give the desired product L2 (3.5 g, 4.649 mmol, 42.86%).LCMS (M+H)⁺=753.0;

1 H NMR (400 MHz, DMSO) b 7.90 (d, J=7.5 Hz, 2H), 7.74-7.64 (m, 2H), 7.44-7.32 (m, 4H), 4.58-4.21 (m, 8H), 4.14-3.74 (m, 4H), 3.68-3.41 (m, 8H), 2.85-2.56 (m, 2H), 1.69-1.28 (m, 15H). 13C NMR (100 MHz, DMSO) b 171.53, 156.10, 143.77, 140.70, 127.62, 127.04, 125.24, 120.10, 80.47, 80.42, 71.66, 71.58, 71.34, 70.18, 65.53, 63.51, 63.36, 54.48, 54.41, 46.63, 27.65, 23.14, 22.38.

Step 2 To a solution of L2 (200 mg, 0.266 mmol) in THF (2 mL) was added the diethylamine (38.86 mg, 0.531 mmol). The reaction mixture was stirred at room temperature for 2 hr. A sample was taken from the reaction mixture, and the LCMS result showed the desired product was found and the starting material was consumed completely. The solvents were evaporated, and the residue was purified by C18 reversed-phase chromatography to give the desired product L3 (120 mg), LCMS (M+H)⁺=531.1.

### Example 2: Preparation of a PEG Unit

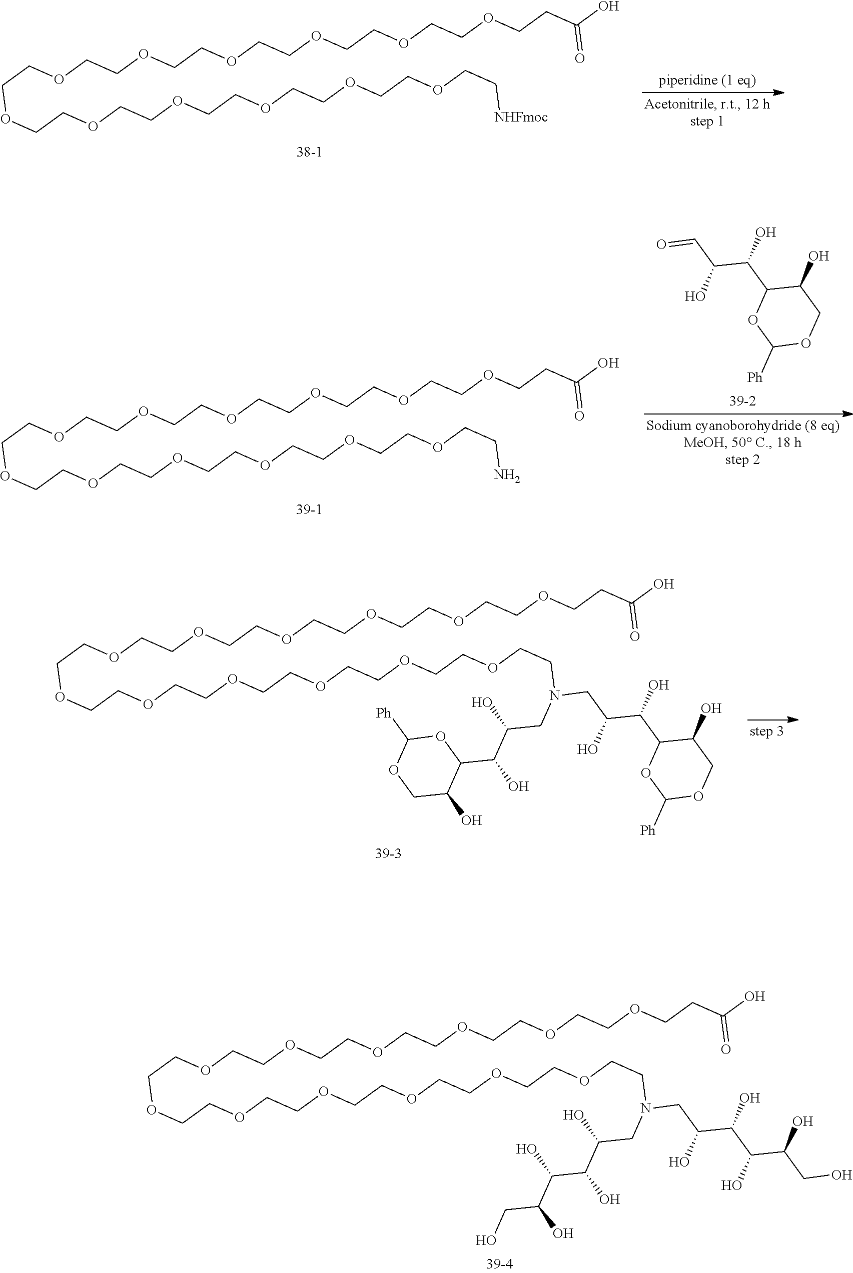

A PEG unit containing linear monosaccharide was prepare as follows:

### Step 1

A solution of compound 38-1 (260 mg, 0.31 mmol) in acetonitrile (3.0 mL) was stirred at r.t. and diethyl amine anhydrous (0.2 mL, 1.941 mmol) was added. The resulting solution was stirred at r.t. for 2 h until LCMS of the solution showed that most of starting material was consumed. Then the solution was concentrated to dryness and the residue was purified by reverse phase column chromatography (12 g C18 column, eluting with 0-50% acetonitrile in water with 0.01% TFA) to give expected fractions of compound 39-1 (170 mg, 0.28 mmol) as a pale yellow oil. LCMS, ESI m/z=618.4 (M+H)⁺;

### Step 2

A clear reaction solution of compound 39-1 (170 mg, 0.28 mmol), 39-2 (217.08 mg, 1.206 mmol) and acetic acid (1.21 mg, 0.020 mmol) in methanol (5 mL) was heated at 50° C. for 30 min, and then NaCNBH₃ (75.98 mg, 1.206 mmol) was added. The resulting solution was stirred at 50° C. under N₂ for 4 hr. Then additional NaCNBH₃ (75.98 mg, 1.206 mmol) and compound 39-2 (217.08 mg, 1.206 mmol) was added and kept stirring at 50° C. overnight. After stirring for 20 hrs, LCMS indicated the reaction was complete. The solvents were evaporated, and the residue was purified by C18 reversed-phase chromatography to give the desired product 39-3 (265 mg, 0.24 mmol). LCMS, ESI m/z=1122.6 (M+H)⁺.

### Step 3

A mixture of compound 39-3 (265 mg, 0.24 mmol) in 6N HCl/THF aqueous was stirred at rt for 3 hours, until LCMS indicated the reaction was complete. The solvents were neutralized with NaHCO₃ aqueous, evaporated, and the residue was purified by C₁₈ reversed-phase chromatography to give the desired product 39-4 (160 mg, 0.17 mmol). LCMS, ESI m/z=946.5 (M+H)⁺.

### Example 3: Preparation of a Drug Linker Containing MMAE (PB003)

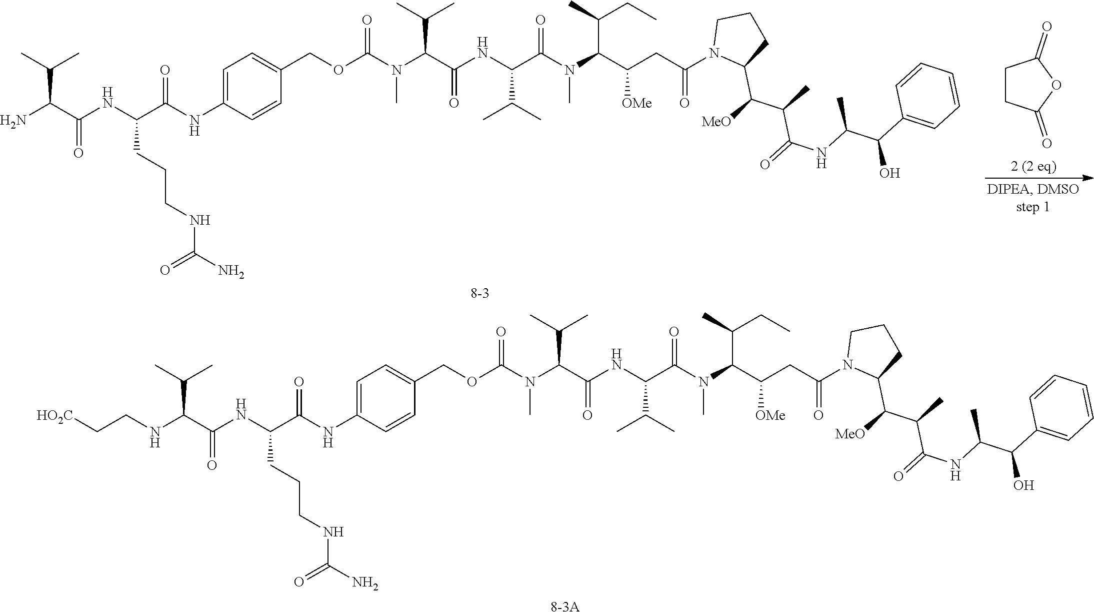

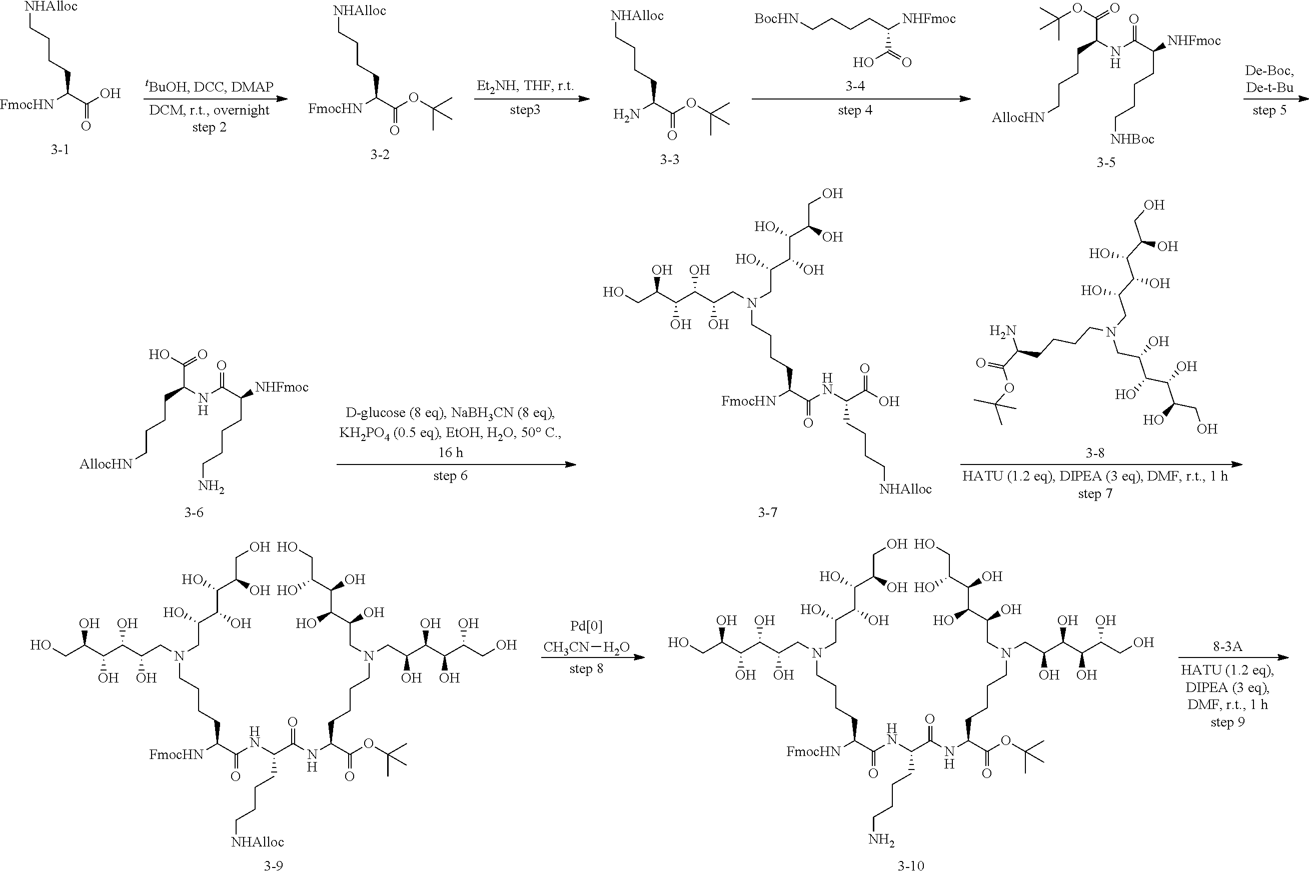

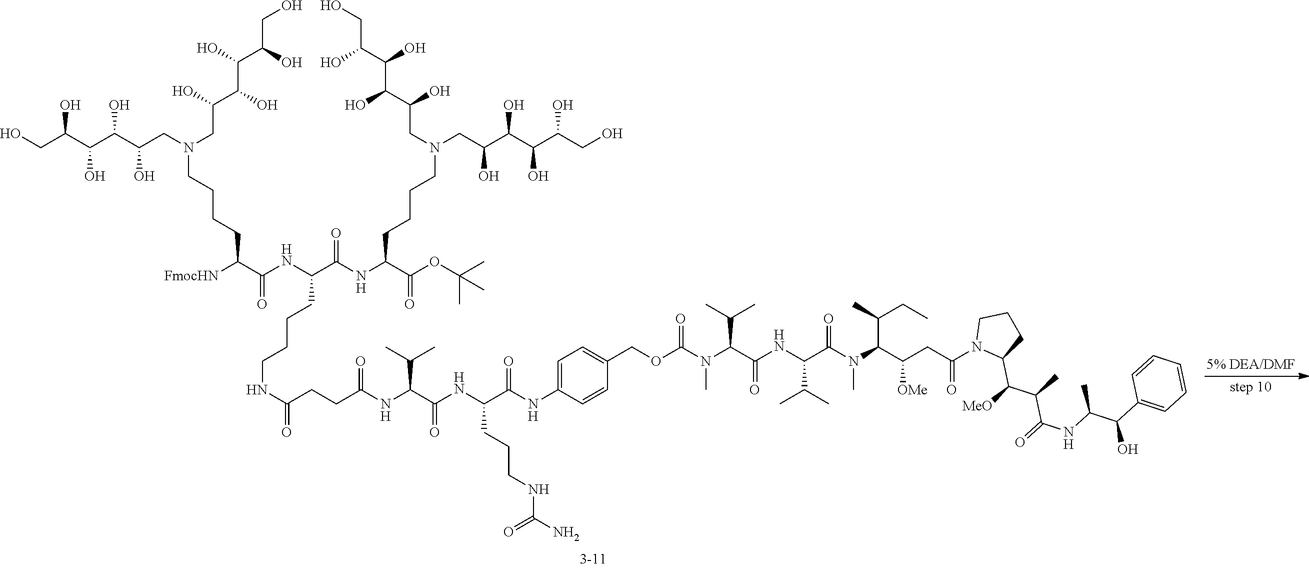

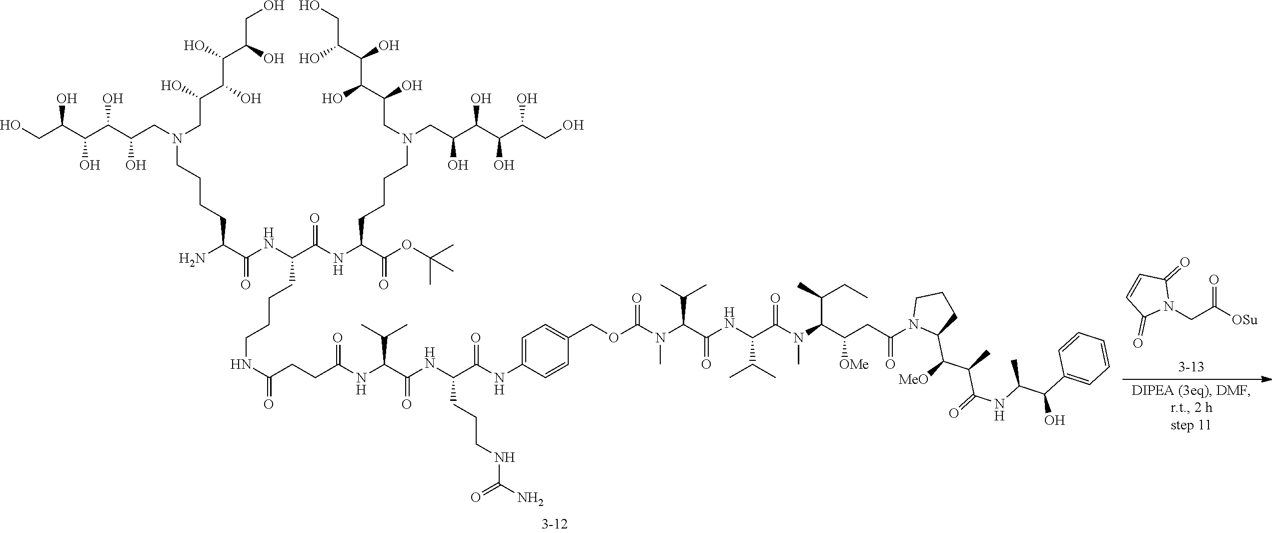

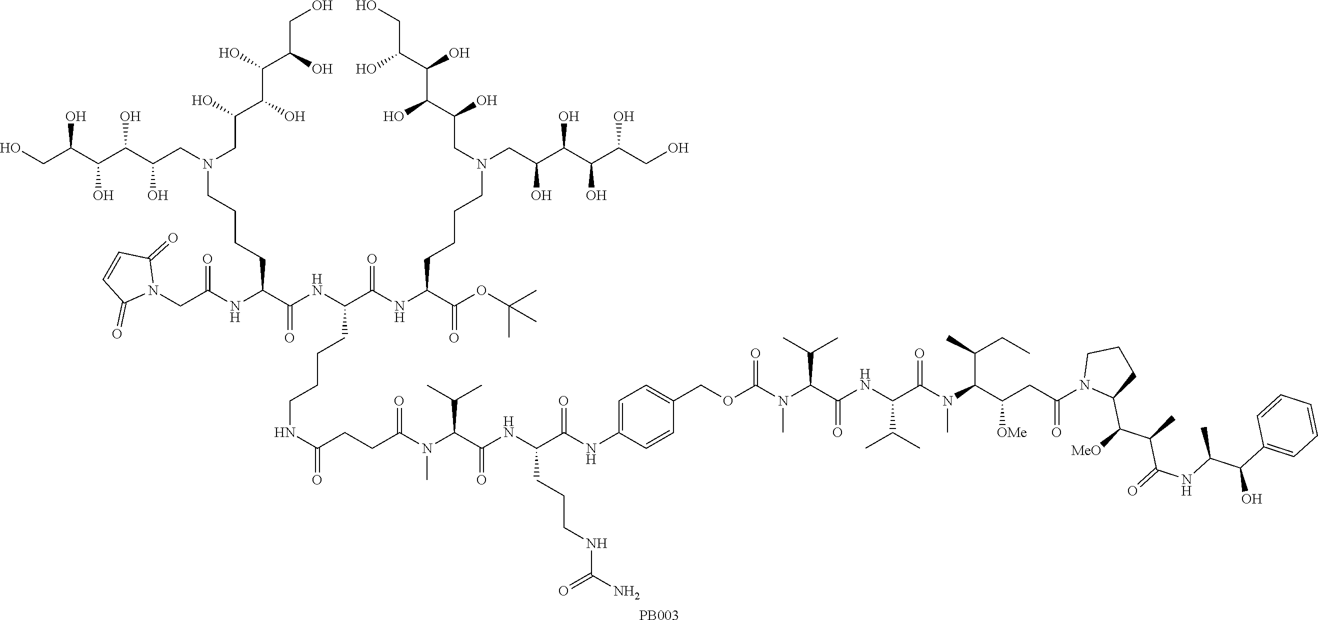

A Drug Linker containing two Sugar units and a cleavable linker attached to MMAE (PB003) was prepared as follows:

### Step 1

A solution of compound 8-3 (30 mg, 0.027 mmol), DIPEA (10.45 mg, 0.081 mmol) in anhydrous DMF (2 mL) was stirred at room temperature, then oxolane-2,5-dione (5.40 mg, 0.054 mmol) was added. The resulting solution was stirred for another 1 hr at room temperature (r.t.) until liquid chromatography mass spectrometry (LCMS) indicated a complete reaction. The mixture was purified directly by reverse phase liquid chromatography (40 g C18 column, eluting with 0-50% acetonitrile in water with 0.01% TFA over 15 min) to give compound 8-3 A (25.8 mg, 0.021 mmol, 78.97%) as a white solid. LCMS: Product (M/2+H)⁺=612.5;

### Step 2

A mixture of compound 3-1 (20.00 g, 44.198 mmol), 2-methylpropan-2-ol (12.600 mL, 132.594 mmol), DCC (13.68 g, 66.297 mmol) and DMAP (1.62 g, 13.259 mmol) in DCM (150 mL) was stirred for 12 hr at room temperature (r.t.) under nitrogen (N₂). After completion of the reaction as judged by LCMS, the reaction mixture was filtered through a Celite pad, and the filtrate was concentrated under the reduced pressure to give a residue which was then purified by silicon-gel flash chromatography (Petroleum Ether: EtOAc=10 1) to afford the compound 3-2 (11.80 g, 23.200 mmol, 52.49%) as a colorless oil. LCMS (M-56+H)⁺=453.1;

1H NMR (400 MHz, DMSO) δ=7.89 (d, J=7.5, 2H), 7.72 (d, J=7.5, 2H), 7.65 (d, J=7.8, 1H), 7.44 (t, J=7.4, 2H), 7.35 (d, J=7.1,

2H), 7.20 (t, J=5.3, 1H), 5.94-5.76 (m, 1H), 5.27 (dd, J=17.2, 1.6, 1H), 5.16 (dd, J=10.5, 1.4, 1H), 4.45 (d, J=5.3, 2H), 4.32 (dd, J=12.3, 4.8, 2H), 4.26-4.20 (m, 1H), 3.84 (d, J=4.9, 1H), 2.97 (dd, J=12.6, 6.4, 2H), 1.66-1.53 (m, 2H), 1.38 (s, 9H), 1.33-1.29 (m, 2H), 1.28-1.23 (m, 2H).

### Step 3

To a solution of compound 3-2 (5 g, 9.837 mmol) in DCM (20 mL) was added Et2NH (4 mL, 38.693 mmol). The reaction was stirred at room temperature for 2 h. The mixture was concentrated and the crude compound 3-3 (2.84 g, 9.921 mmol, 100%) was used in the next step directly. ESI m/z: 287.3(M+H)⁺.

### Step 4

To a solution of compound 3-3 (2.84 g, 9.917 mmol) in DMF (15 mL) was added compound 3-4 (5.58 g, 11.901 mmol), DIPEA (2.56 g, 19.834 mmol) and HATU (3.77 g, 9.917 mmol). The reaction was stirred at room temperature for 1 h. Then the mixture was concentrated and purified by reverse phase separation (C18 column, eluting with 0-87% acetonitrile in water with TFA) to afford the compound 3-5 (5.2 g, 7.056 mmol,71.15%) as white solid. ESI m/z: 759.4(M+Na)⁺.

### Step 5

To a solution of compound 3-5 (5.2 g, 7.056 mmol) in DCM (12 mL) was added TFA (12 mL, 1199.474 mmol). The reaction was stirred at room temperature for 4 h. Then the mixture was concentrated and purified by reverse column separation (C18 column, eluting with 0-44% acetonitrile in water with TFA) to yield compound 3-6 (2.4 g, 4.133 mmol, 58.57%) as a white solid. ESI m/z: 581.3(M+H)⁺.

### Step 6

To a solution of compound 3-6 (2.40 g, 4.133 mmol) in EtOH (35 mL) and H₂O (5 mL) was added D-glucose (5.93 g, 32.919 mmol), KH₂PO4 (0.020 mL, 0.344 mmol) and NaBH₃CN (2.08 g, 33.099 mmol). The reaction was stirred at 50° C. for 18 h. The reaction was stirred at room temperature for 4 h. Then the mixture was concentrated and purified by reverse column separation (C18 column, eluting with 0-44% acetonitrile in water with TFA) to yield compound 3-7 (2.0 g, 2.200 mmol, 53.48%) as white solid. ESI m/z: 910.4(M+H)⁺.

### Step 7

To a solution of compound 3-7 (1.00 g, 1.100 mmol) in DMF (15 mL) was added HATU (0.50 g, 1.320 mmol) and DIPEA (0.43 g, 3.300 mmol). The mixture was stirred for 10 min and then compound 3-8 (0.58 g, 1.099 mmol) was added. The reaction was stirred for 1 h at room temperature. Then the mixture was concentrated and purified by reverse phase column separation (C18 column, eluting with 0-34% acetonitrile in water with TFA) to yield compound 3-9 (0.47 g, 0.334 mmol, 30.37%). ESI m/z: 711.5(M/2+H)⁺.

### Step 8

A clear solution of compound 3-9 (10 mg, 0.007 mmol), Pd(PPh3)4 (0.41 mg, 0.000 mmol) and diethyl amine anhydrous (0.001 mL, 0.014 mmol) in MeCN (0.5 mL) and water (0.1 mL) was stirred at room temperature for 2 hr under N₂. LCMS indicated all starting material was consumed, and desired mass of product (fragment mass 669 in LCMS) was detected. The mixture was purified by reverse phase liquid chromatography (12 g C18 column, eluting with 0-35% acetonitrile in water with 0.01% TFA) to yield product 3-10 as a white solid. LCMS: Product (M/2+H)⁺=669.5; purity 65% (214 nm).

### Step 9

A solution of compound 3-10 (27 mg, 0.024 mmol), 8-3A (54.67 mg, 0.048 mmol) and DIPEA (3.10 mg, 0.024 mmol) in anhydrous DMF (1.8 mL) was stirred at room temperature for 5 min, then a solution of HATU (9.14 mg, 0.024 mmol) in anhydrous DMF (0.2 mL) was added. The resulting solution was stirred for another1 hr at r.t. until LCMS indicated complete reaction. The reaction mixture was purified directly by reverse phase liquid chromatography (40 g C18 column, eluting with 0-70% acetonitrile in water with 0.01% TFA over 15 min) to yield product 3-11 (40.5 mg, 0.016 mmol) as a white solid. LCMS (M/3+H)⁺=848.5;

### Step 10

A solution of compound 3-11 (45 mg, 0.018 mmol) in DMF (0.95 mL) was stirred at r.t. and diethyl amine anhydrous (0.05 mL, 0.485 mmol) was added. After addition, the resulting solution was kept stirring at rt. for another 1 hr until LCMS showed that the reaction was completed. Volatiles (especially DEA) were evaporated off to give a crude product, purified directly by reverse phase liquid chromatography (12 g C18 column, eluting with 0-50% acetonitrile in water with 0.01% TFA over 15 min) to give expected fractions, which were lyophilized to yield product 3-12 (30 mg, 0.013 mmol, 73.05%) as a white solid. LCMS (M/3+H)⁺=774.4;

### Step 11

A solution of compound 3-12 (20 mg, 0.009 mmol) and DIPEA (3.34 mg, 0.026 mmol) in anhydrous DMF (0.8 mL) was stirred at room temperature for 5 min, then a solution of 2,5-dioxopyrrolidin-1-yl 2-(2,5-dioxo-2,5-dihydro-1H-pyrrol-1-yl)acetate (4.35 mg, 0.017 mmol) in anhydrous DMF (0.2 mL) was added dropwise over 2 min. The resulting solution was stirred for another 2 hr at r.t., then monitored by LCMS; the desired product was formed as majority of the reaction product. The reaction was quenched with one drop of water and then purified directly by Prep-HPLC (Mobile Phase: A: Water (0.01% TFA) B: acetonitrile (0.01% TFA); Gradient Phase: 5% of B increasing to 95% of B with 15 min; Flow Rate: 1.0 mL/min; Column: SunFire C18, 4.6*50 mm, 3.5 μm; Column Temperature: 50° C. Detectors: ADC ELSD, DAD (214 nm and 254 nm) to yield product PB003 (7 mg, 0.003 mmol) as a white solid. LCMS (M/3+H)⁺=820.1

1H NMR (400 MHz, DMSO-d6) δ 9.86-9.78 (m, 1H), 8.49-8.31 (m, 3H), 8.26-8.06 (m, 3H), 8.01-7.83 (m, 3H), 7.66-7.59 (m, 2H), 7.32-7.25 (m, 6H), 7.20-7.15 (m, 1H), 7.09 (s, 1H), 6.05-5.98 (m, 1H), 5.45-5.35 (m, 6H), 5.13-4.95 (m, 2H), 4.87-4.71 (m, 4H), 4.71-4.41 (m, 13H), 4.35-4.13 (m, 7H), 4.10-4.04 (m, 2H), 3.99-3.94 (m, 6H), 3.80-3.77 (m, 1H), 3.73-3.69 (m, 4H), 3.67-3.43 (m, 21H), 3.24-3.17 (m, 17H), 3.12-2.97 (m, 7H), 2.89-2.83 (m, 3H), 2.44-2.24 (m, 7H), 2.16-1.91 (m, 5H), 1.84-1.42 (m, 19H), 1.38 (s, 9H), 1.34-1.24 (m, 9H), 1.06-0.98 (m, 6H), 0.89-0.75 (m, 26H) ppm. Proton signals of two molecule of TFA are included.

### Example 4: Preparation of a Drug-Linker Having Two Sugar Units and a Cleavable Linker Attached to MMAE (PB004)

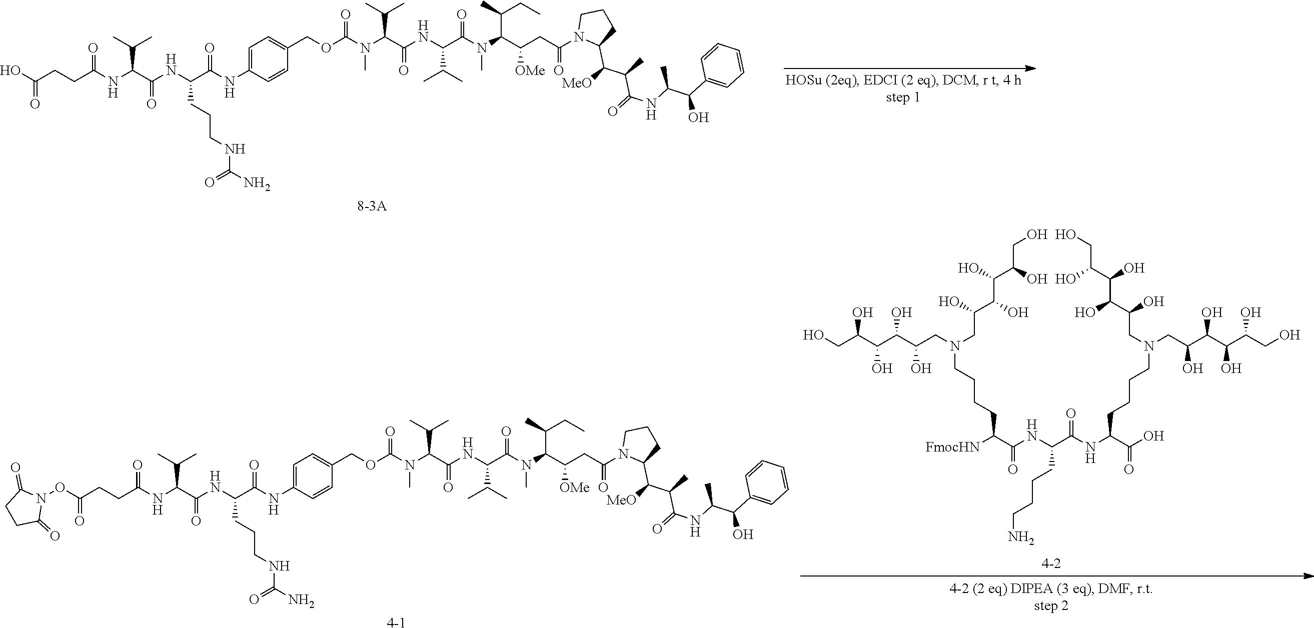

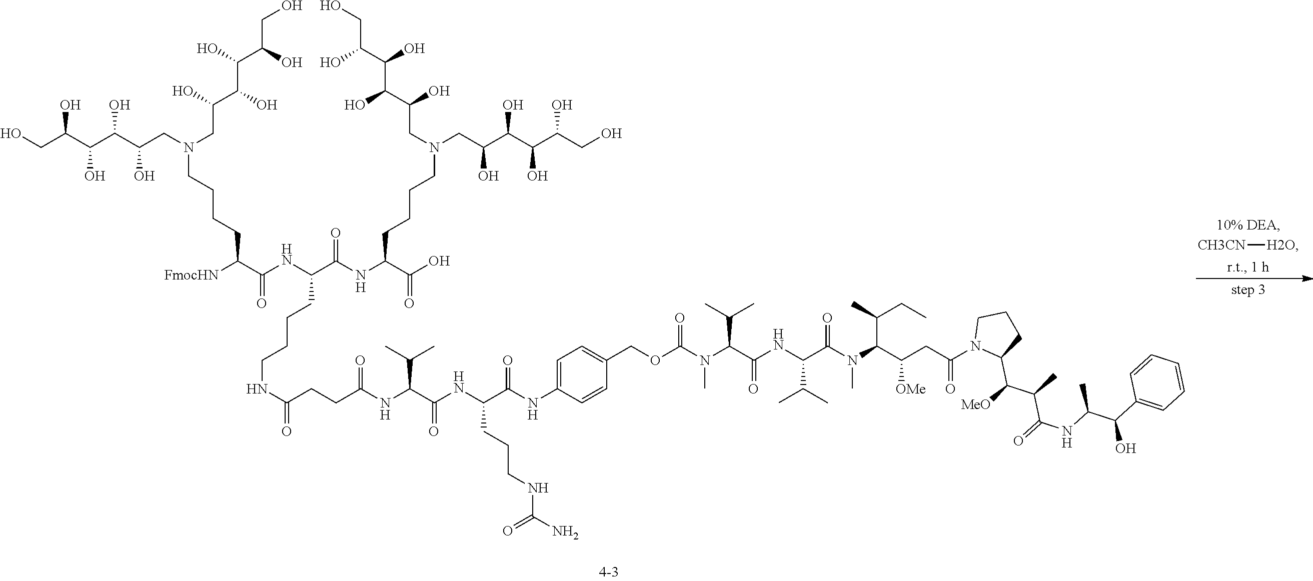

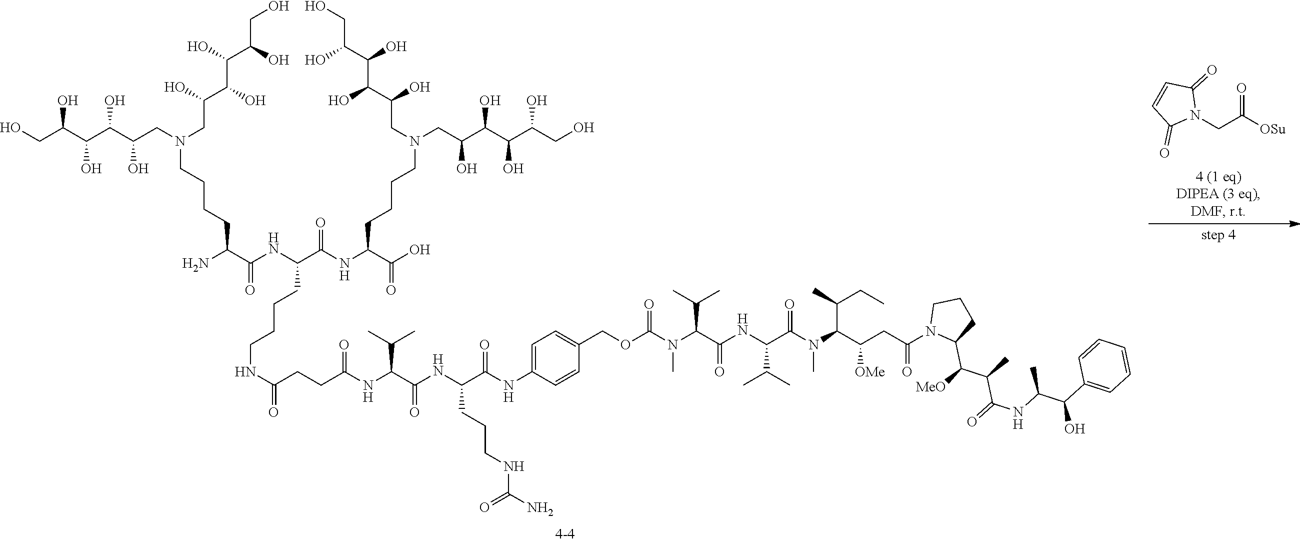

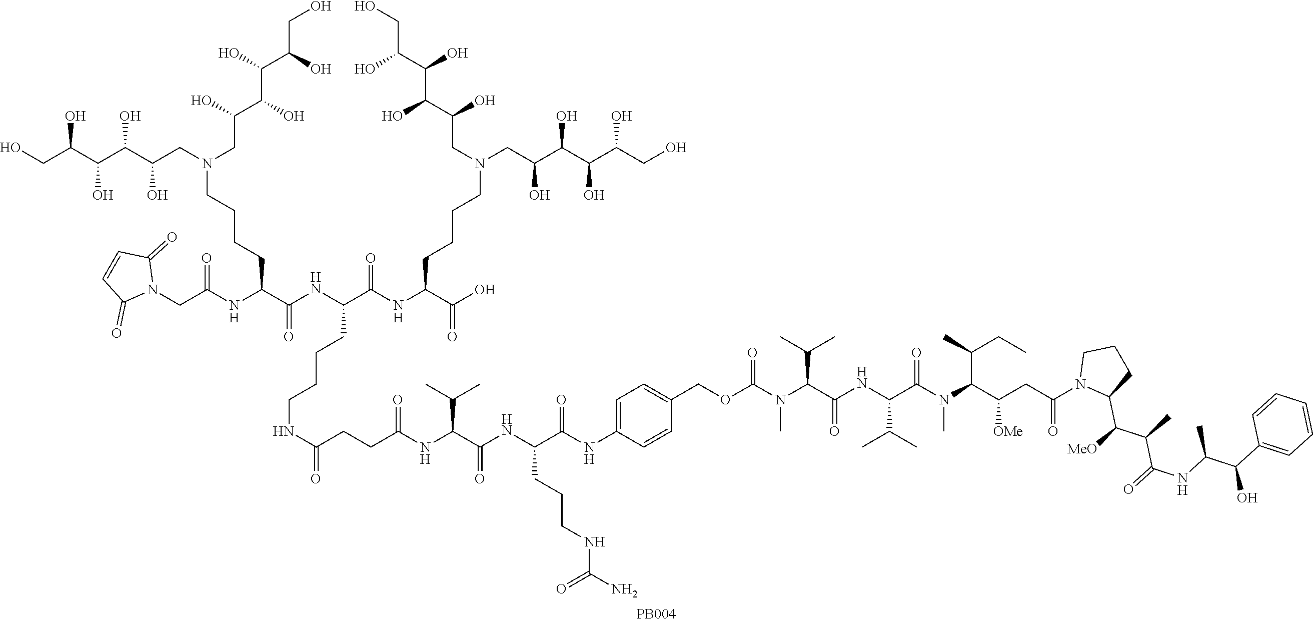

A Drug Linker containing Sugar units and a cleavable linker attached to MMAE (PB004) was prepared as follows:

### Step 1

A solution of compound 8-3 A (50 mg, 0.041 mmol) and HOSu (7.05 mg, 0.061 mmol) in anhydrous DCM (5 mL) was stirred at room temperature, and then a solution of EDCl (11.75 mg, 0.061 mmol) was added. The resulting solution was stirred for another 1.5 hr at r.t. until LCMS indicated a complete reaction. The reaction solution was diluted with more DCM (10 mL) and washed with water. The organic layer was collected and dried over sodium sulfate, then condensed to dryness to give crude NHS ester 4-1 (50 mg, 0.038 mmol, 92.65%) as a white solid, which was used directly in next step (refer to N200897-071). LCMS (M/2+H)⁺=661.0; purity=96% (254 nm).

### Step 2

The crude compound 4-1 (50 mg, 0.038 mmol) from last step was dissolved in anhydrous DMF (2 mL), then DIPEA (14.65 mg, 0.114 mmol) and compound 4-2 (48.52 mg, 0.038 mmol) was added. The resulting solution was stirred at room temperature for 2 h until LCMS indicated all starting amine was consumed. The reaction solution was purified directly by reverse phase liquid chromatography (40 g C18 column, eluting with 0-70% acetonitrile in water with 0.01% TFA over 15 min) to yield compound 4-3 (50 mg, 0.020 mmol, 53.10%) as a white solid. LCMS: m/z=829.8 (M/3+H)⁺;

### Step 3

A suspension of compound 4-3 (50 mg, 0.022 mmol) in acetonitrile (2 mL) was stirred at r.t. and water (0.5 mL) was added to improve the solubility. The material was dissolved into a clear solution. Then diethyl amine anhydrous (0.2 mL, 1.941 mmol) was added. The resulting solution was stirred at r.t. for 1 h. until the LCMS showed that the reaction was completed. Then the solution was concentrated to dryness to remove most of the diethyl amine, and then redissolved in acetonitrile and water and lyophilized to yield crude product 4-4 (48 mg, 0.021 mmol, 96.94%) as a white solid, which was used directly in the next step. LCMS: m/z=739.0 (inclusive fragment piece, (2263/3)+H=755 is expected);

### Step 4

A solution of compound 4-4 (47 mg, 0.021 mmol) and DIPEA (8.03 mg, 0.062 mmol) in anhydrous DMF (0.8 mL) was stirred at room temperature for 5 min, then a solution of 2,5-dioxopyrrolidin-1-yl 2-(2,5-dioxo-2,5-dihydro-1H-pyrrol-1-yl)acetate (5.23 mg, 0.021 mmol) in anhydrous DMF (0.2 mL) was added dropwise by syringe. The resulting solution was stirred for another 0.5 hr at r.t. until LCMS indicated all starting amine was consumed and the mass of desired product was detected. The resulting solution was stirred at room temperature for 2 h until LCMS indicated all starting amine was consumed. The reaction solution was purified directly by Prep-HPLC (eluting with gradient with 0.01% TFA over 20 min) to yield product PB004 (10 mg, 0.004 mmol, 20.06%) as a white solid. LCMS: m/z=801.4 (M/3+H)⁺;

1H NMR (400 MHz, DMSO-d6) δ 12.56 (s, 1H), 9.77 (s, 1H), 8.52-8.23 (m, 3H), 8.19-7.84 (m, 6H), 7.67-7.61 (m, 2H), 7.35-7.24 (m, 6H), 7.19-7.13 (m, 1H), 7.09 (s, 2H), 6.05-6.02 (m, 1H), 5.52-5.36 (m, 6H), 5.13-4.94 (m, 2H), 4.89-4.73 (m, 4H), 4.69-4.36 (m, 13H), 4.29-4.23 (m, 6H), 4.14-4.09 (m, 2H), 4.05-3.93 (m, 6H), 3.79-3.76 (m, 1H), 3.72-3.68 (m, 4H), 3.65-3.43 (m, 21H), 3.24-3.05 (m, 17H), 3.01-2.91 (m, 7H), 2.89-2.83 (m, 3H), 2.44-2.23 (m, 7H), 2.14-1.94 (m, 5H), 1.86-1.41 (m, 19H), 1.37-1.20 (m, 9H), 1.05-0.97 (m, 6H), 0.88-0.75 (m, 26H) ppm. Proton signals of two molecule of TFA are included.

### Example 5: Preparation of a Drug-Linker Having One Sugar Unit and a Cleavable Linker Attached to MMAE (PB008)

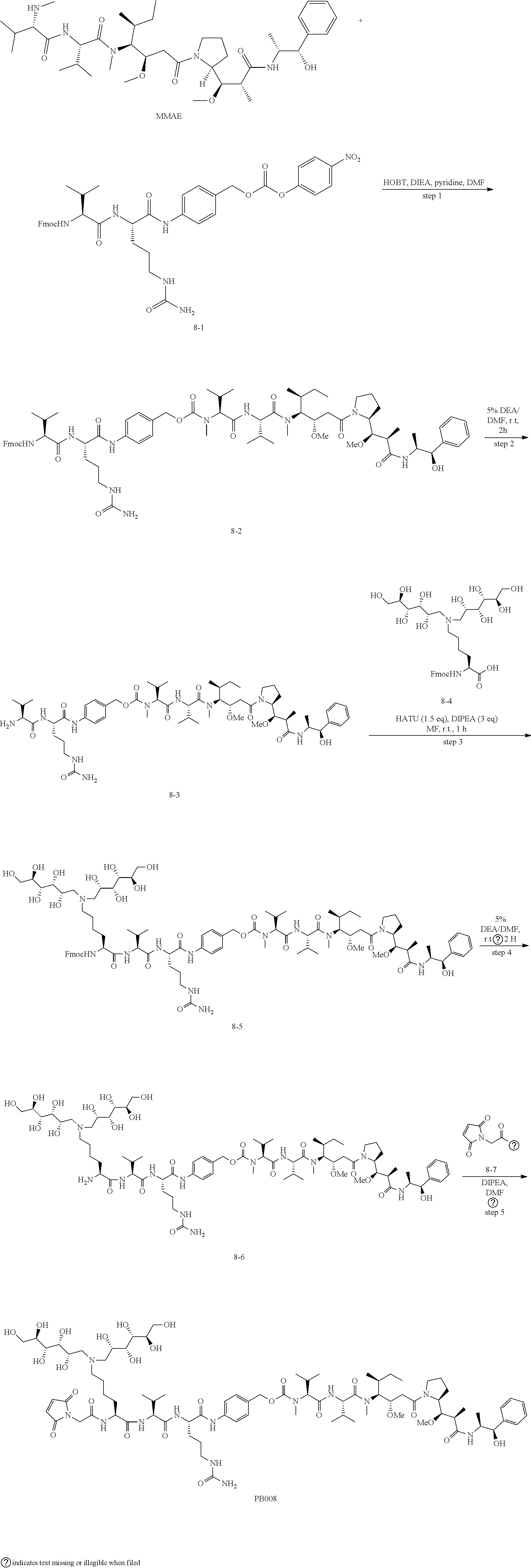

A Drug-Linker having one Sugar Unit and a cleavable linker attached to MMAE (PB008) was prepared as follows:

### Step 1

To a solution of compound 8-1 (155.50 mg, 0.203 mmol) in DMF (10 mL), HOBt (26.35 mg, 0.195 mmol) and MMAE (140 mg, 0.195 mmol) was added at 0° C. The reaction mixture was stirred at 0° C. for 15 min, Pyridine (3 mL) and DIEA ethyldiisopropylamine (0.039 mL, 0.234 mmol) were added at 0° C., and the reaction mixture was stirred at 0° C. for 30 min. The reaction mixture was allowed to warm to r.t. The reaction mixture was stirred at r.t. for 36 hr. After removing DIEA and pyridine, the residue was purified by Prep-HPLC to yield the desired product 8-2 (62 mg, 0.045 mmol). LCMS ((M+2H)/2)⁺=673.4;

### Step 2

To a solution of compound 8-2 (60 mg, 0.045 mmol) in DMF (0.95 mL) was added diethyl amine anhydrous (0.05 mL, 0.485 mmol). Then the resulting solution was stirred at room temperature for 1 h until LCMS indicated complete deprotection. The completed reaction solution was purified by reverse phase column chromatography (C18 column, eluting with 0-70% acetonitrile in water with 0.01% TFA) to get the desired fractions containing compound 8-3, which were lyophilized to yield a TFA salt of compound 8-3 (40 mg, 0.036 mmol, 79.85%) as a white solid. LCMS (M/2+H)⁺=562.5; 1H NMR (400 MHz, DMSO-d6) δ 10.20 (s, 1H), 8.69 (d, J=7.6 Hz, 1H), 8.30 (brs, 2H), 8.17-8.07 (m, 3H), 7.90 (d, J=8.4 Hz, 0.5H), 7.64 (d, J=8.4 Hz, 0.5H), 7.59-7.57 (m, 2H), 7.37-7.24 (m, 6H), 7.20-7.13 (m, 1H), 6.05-6.02 (m, 1H), 5.48 (s, 2H), 5.43-5.34 (m, 1H), 5.12-4.97 (m, 2H), 4.78-4.23 (m, 4H), 4.04-3.93 (m, 2H), 3.80-3.52 (m, 2H), 3.25-3.06 (m, 7H), 2.98-2.83 (m, 10H), 2.43-2.39 (m, 1H), 2.26-2.22 (m, 1H), 2.14-1.99 (m, 3H), 1.99-1.85 (m, 1H), 1.85-1.67 (m, 4H), 1.67-1.37 (m, 5H), 1.37-1.25 (m, 1H), 1.16 (t, J=7.2 Hz, 3H), 1.05-1.00 (m, 6H), 0.98-0.93 (m, 6H), 0.86-0.73 (m, 17H) ppm.

### Step 3

A solution of compound 8-3 (17.96 mg, 0.026 mmol), compound 8-4 and DIPEA (9.99 mg, 0.077 mmol) in anhydrous DMF (0.8 mL) was stirred at room temperature for 5 min, and then a solution of HATU (14.71 mg, 0.039 mmol) in anhydrous DMF (0.2 mL) was added. The resulting solution was stirred for another 1 hr at r.t. until LCMS indicated complete reaction. The reaction solution was purified directly by reverse phase liquid chromatography (C18 column, eluting with 0-70% acetonitrile in water with 0.01% TFA over 15 min) to yield compound 8-5 (36.2 mg, 0.020 mmol, 77.26%) as a white solid. LCMS (M/2+H)⁺=902.1; 1H NMR (400 MHz, DMSO-d6)

### Step 4

A solution of compound 8-5 (50 mg, 0.028 mmol) in DMF (0.95 mL) was stirred at r.t. and DEA (0.05 mL, 0.313 mmol) was added. The resulting solution was stirred at r.t. for 2 hr. The LCMS of the solution showed that the reaction was completed. The completed reaction solution was purified directly by reverse phase liquid chromatography (12 g C18 column, eluting with 0-60% acetonitrile in water with 0.01% TFA over 15 min) to give the expected fractions, which were lyophilized to yield product 8-6 (35 mg, 0.022 mmol, 79.85%) as a white solid. LCMS (M/2+H)⁺=790.6;

### Step 5

A solution of compound 8-6 (30 mg, 0.019 mmol), DIPEA (7.35 mg, 0.057 mmol) in anhydrous DMF (2 mL) was stirred at r.t., then compound 8-7 (2,5-dioxopyrrolidin-1-yl 2-(2,5-dioxo-2,5-dihydro-1H-pyrrol-1-yl)acetate) (9.58 mg, 0.038 mmol) was added. The resulting solution was stirred for another 1 hr to achieve complete conversion. The completed reaction solution was purified by Prep-HPLC Mobile Phase: A: Water (0.01% TFA) B: acetonitrile (0.01% TFA); Gradient Phase: 5% of B increasing to 95% of B with 15 min; Flow Rate: 1.0 mL/min; Column: SunFire C18, 4.6*50 mm, 3.5 μm; Column Temperature: 50° C. Detectors: ADC ELSD, DAD (214 nm and 254 nm) to afford TFA salt of PB008 (20 mg, 0.012 mmol, 61.34%) as a white solid. LCMS (M/2+H)⁺=859.0;

1H NMR (400 MHz, DMSO-d6) δ 10.03 (s, 1H), 8.42-8.40 (m, 2H), 8.33-8.31 (m, 0.5H), 8.20-8.18 (m, 1H), 8.11-8.09 (m, 0.5H), 7.92-7.84 (m,1.5H),7.66-7.63 (m, 0.5H), 7.59-7.56 (m, 2H), 7.36-7.24 (m, 6H), 7.20-7.14 (m, 1H), 7.10 (s, 2H), 6.06-6.01 (m, 1H), 5.54-5.33 (m, 4H), 5.13-4.95 (m, 2H), 4.87-4.34 (m, 10H), 4.30-4.17 (m, 2H), 4.09 (s, 2H), 4.04-3.92 (m, 4H), 3.80-3.77 (m, 0.5H), 3.70-3.65 (m, 2H), 3.61-3.58 (m, 9.5H), 3.33-3.24 (m, 2H), 3.23-3.07 (m, 13H), 3.03-2.89 (m, 5H), 2.89-2.83 (m, 3H), 2.44-2.39 (m, 1H), 2.30-2.21 (m, 1H), 2.16-2.05 (m, 2H), 1.99-1.91 (m, 2H), 1.84-1.22 (m, 18H), 1.06-0.97 (m, 6H), 0.89-0.75 (m, 26H) ppm. Proton signals of one molecule of TFA are included.

### Example 6: Preparation of a Drug-Linker Having Two Sugar Units and a Cleavable Linker Attached to Exatecan (PB026)

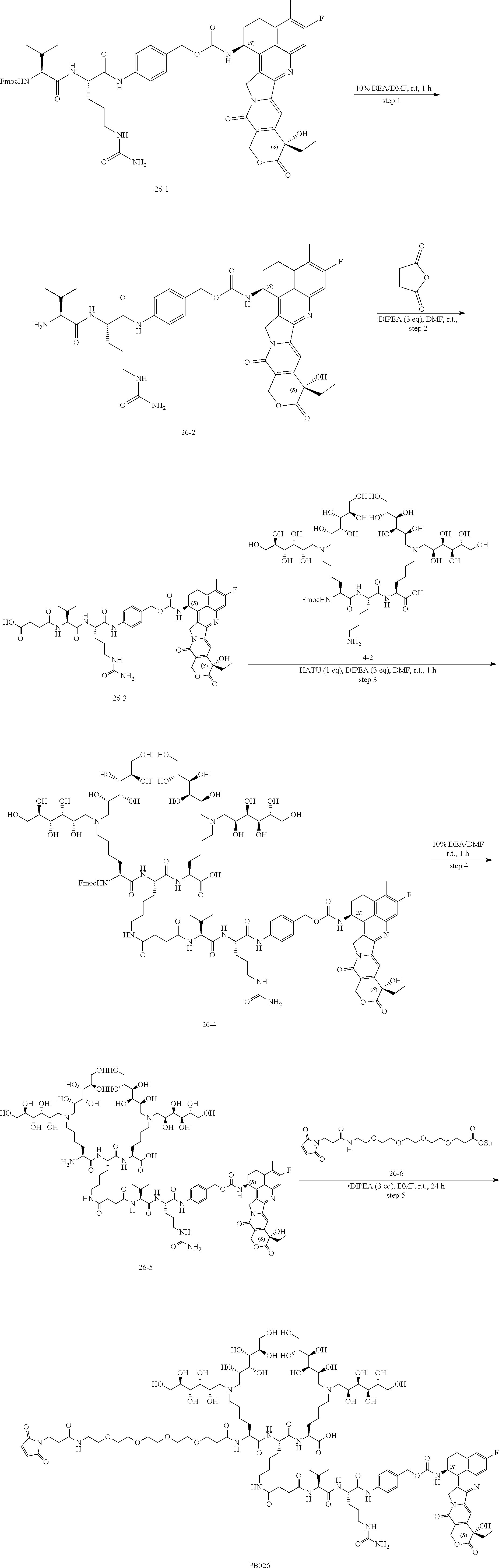

A Drug-Linker having two Sugar units and a cleavable linker attached to exatecan (PB026) was prepared as follows:

### Step 1

A solution of compound 26-1 (475 mg, 0.447 mmol) in DMF (3.6 mL) was stirred at r.t. and diethyl amine anhydrous (0.4 mL, 3.883 mmol) was added. The resulting solution was stirred at r.t. for 1 h. Then LCMS of the solution showed that the reaction was completed. The reaction solution was purified directly by reverse phase liquid chromatography (120 g C18 column, eluting with 0-80% acetonitrile in water with 0.01% TFA over 15 min) to yield product 26-2 (260 mg, 0.309 mmol, 69.24%) as a white solid. LCMS: m/z=841.1 (M+H)⁺

### Step 2

A solution of compound 26-2 (150 mg, 0.178 mmol) and oxolane-2,5-dione (35.70 mg, 0.357 mmol) in anhydrous DMF (2 mL) was stirred at room temperature, and then DIPEA (69.03 mg, 0.535 mmol) was added. The resulting solution was stirred at room temperature for 1 h until LCMS indicated all starting amine was consumed. The completed reaction solution was purified directly by reverse phase liquid chromatography (40 g C18 column, eluting with 0-70% acetonitrile in water with 0.01% TFA over 15 min) to yield compound 26-3 (140 mg, 0.149 mmol, 83.41%) as a white solid. LCMS: m/z=963.5(M+Na)⁺

### Step 3

A solution of compound 26-3 (100 mg, 0.106 mmol), DIPEA (41.02 mg, 0.318 mmol) and HATU (40.30 mg, 0.106 mmol) in anhydrous DMF (1 mL) was stirred at room temperature for 10 min, and then a solution of compound 4-2 (271.57 mg, 0.212 mmol) in anhydrous DMF (1 mL) was added dropwise by syringe. After addition, the resulting solution was stirred for another 1 hr at r.t. until LCMS indicated the starting acid was almost consumed and the reaction was completed. The reaction solution was purified by reverse phase liquid chromatography (40 g C18 column, eluting with 0-70% acetonitrile in water with 0.01% TFA) to yield product 26-4 (100 mg, 0.045 mmol, 42.64%) as a white solid. LCMS: ESI m/z=735.8 (M/2+H)⁺;

### Step 4

To a suspension of compound 26-4 (100 mg, 0.045 mmol) in acetonitrile (0.9 mL) was added water (0.9 mL) to help dissolve most of the material. Then diethyl amine anhydrous (0.2 mL, 1.941 mmol) was added to the solution and it was stirred at r.t. for 2 hours. During this process, a sticky oil precipitated on the bottom of the flask. The reaction was monitored by LCMS, the starting material was consumed and the desired product was detected as a major peak. The solution was concentrated to dryness and the residue was washed with petroleum ether to remove most of the nonpolar impurities. The undissolved solid was filtered and collected, then dissolved in acetonitrile and water (1:1) and lyophilized in a freeze dryer to yield compound 26-5 (60 mg, 0.030 mmol, 66.72%) as a white solid, which was used directly in the next step.

LCMS: ESI m/z=661.5 (M/3+H)⁺;

### Step 5

A mixture of compound 26-5 (30 mg, 0.015 mmol) and DIPEA (5.86 mg, 0.045 mmol) in anhydrous DMF (1 mL) was stirred at room temperature for 20 min, and solid was observed suspending in the solution, then additional DMF (1 mL) was added, followed by the addition of compound 26-6 (7.77 mg, 0.015 mmol). The resulting solution was stirred for at r.t. for 24 hr until LCMS and HPLC indicated starting amine was substantially consumed. The reaction solution was purified directly by Prep-HPLC (Mobile Phase: A: Water (0.01% FA) B: acetonitrile (0.01% FA); Gradient Phase: 5% of B increasing to 95% of B with 15 min; Flow Rate: 1.0 mL/min; Column: SunFire C18, 4.6*50 mm, 3.5 μm; Column Temperature: 50° C. Detectors: DAD (214 nm and 254 nm) to yield desired product PB026 (9.1 mg, 0.004 mmol, 25.26%) as a white solid. LCMS: ESI m/z=794.3 (M/3+H)⁺;

1H NMR (400 MHz, DMSO-d6) δ 9.73 (s, 1H), 8.38-8.31 (m, 1H), 8.20-8.16 (m, 2H), 8.08-8.00 (m, 4H), 7.89-7.82 (m, 1H), 7.78 (d, J=11.2 Hz, 1H), 7.65 (d, J=8.0 Hz, 2H), 7.41-7.35 (m, 2.6H), 7.31 (s, 1H), 7.01-7.00 (m, 1.4H), 6.52 (brs, 1H), 6.09-6.01 (m, 1H), 5.45 (s, 4H), 5.29 (s, 4H), 5.08 (s, 2H), 4.36-4.27 (m, 2H), 4.15-4.12 (m, 1H), 4.09-4.03 (m, 0.5H), 3.96-3.88 (m, 1H), 3.88-3.82 (m, 0.5H), 3.77-3.70 (m, 1H), 3.64-3.57 (m, 12H), 3.50-3.47 (m, 17H), 3.43-3.40 (m, 10H), 3.40-3.22 (m, 11H), 3.16-3.12 (m, 4H), 3.05-2.90 (m, 11H), 2.72-2.50 (m, 3H), 2.38-2.31 (m, 11H), 2.24-2.12 (m, 5H), 2.08-1.98 (m, 2H), 1.93-1.87 (m, 2H), 1.84-1.55 (m, 8H), 1.55-1.23 (m, 18H), 1.13-1.07 (m, 2H), 1.01-0.96 (m, 3H), 0.89-0.85 (m, 9H) ppm.

### Example 7: Preparation of a Drug Linker Containing Two Sugar Units and a Cleavable Linker Attached to Exatecan (PB037)

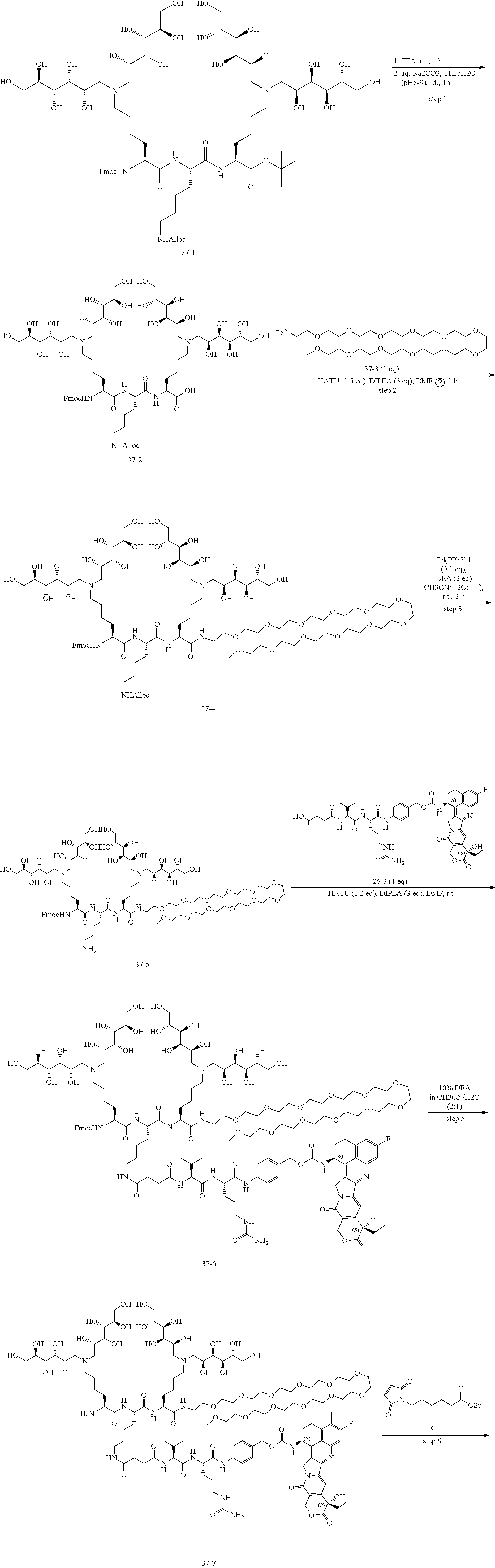

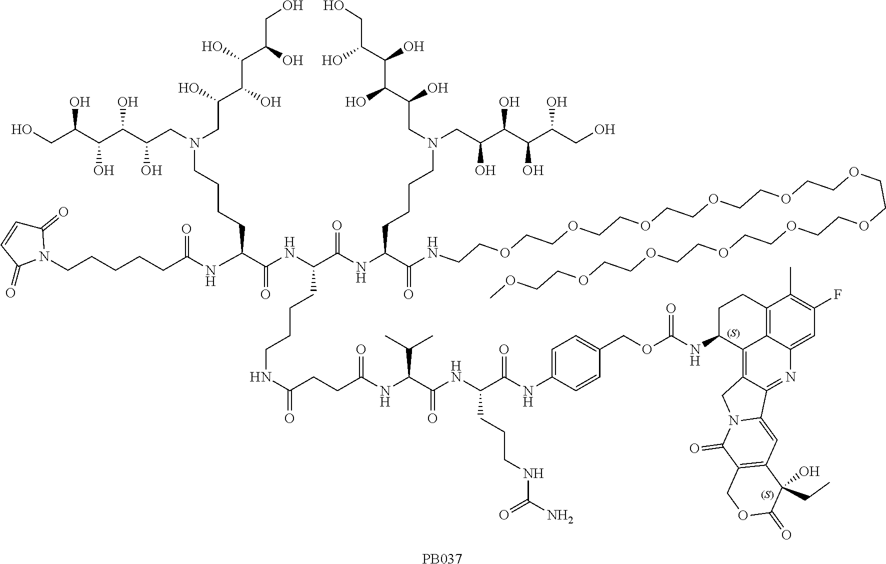

A Drug-Linker containing two Sugar units and a cleavable linker attached to exatecan (PB037) was prepared as follows:

### Step 1

A solution of compound 37-1 (200 mg, 0.141 mmol) in TFA (2 mL) was stirred at r.t. for 2 hr. LCMS of the mixture showed that the reaction was completed and all starting material was consumed and the desired product (mass 640=1280/2) was formed. The completed reaction solution was condensed to dryness and then redissolved in THF (4 mL) and water (1 mL), and treated with saturated aqueous sodium carbonate solution to adjust the pH to 8-9. The resulting solution was stirred at room temperature for 1 h to achieve completion. The solution was then neutralized with diluted TFA and condensed, and the residue was purified by reverse phase liquid chromatography (018 column, eluting with 0-30% acetonitrile in water with 0.01% TFA for 15 min) to yield the expected product 37-2 (180 mg, 0.132 mmol, 93.70%) as a white solid after lyophilization. LCMS, ESI m/z=683.4 (M/2+H)⁺;

### Step 2

A solution of compound 37-2 (180 mg, 0.132 mmol), HATU (75.27 mg, 0.198 mmol) and DIPEA (51.07 mg, 0.396 mmol) in anhydrous DMF (2 mL) was stirred at room temperature for 5 min and compound 37-3 (73.86 mg, 0.132 mmol) was added. The resulting solution was stirred for another 1 hr at r.t. until LCMS indicated a complete reaction. The reaction solution was purified directly by reverse phase liquid chromatography (40 g C18 column, eluting with 0-70% acetonitrile in water with 0.01% TFA over 15 min) to yield compound 37-3 (220 mg, 0.118 mmol, 89.48%) as a white solid. LCMS, ESI m/z=954.5 (M/2+Na)⁺;

### Step 3

A solution of compound 37-3 (220 mg, 0.115 mmol) and Pd(PPh3)4 (133.30 mg, 0.115 mmol) in anhydrous acetonitrile (1 mL) and water (1 mL) was stirred at room temperature, and then diethyl amine anhydrous (0.024 mL, 0.231 mmol) was added immediately. The resulting solution was stirred for another 2 hr at r.t. until LCMS indicated all starting material was consumed and the mass of the desired product was detected. The resulting solution was concentrated under reduced pressure to remove solvent and diethyl amine. The residue was then purified directly by Prep-HPLC (eluting with gradient with 0.01% TFA over 20 min) to yield compound 37-4 (160 mg, 0.088 mmol, 76.08%) as a white solid. LCMS, m/z=608.6 (M/3+H), 912.2 (M/2+H)⁺;

### Step 4

A solution of compound 37-4 (30.97 mg, 0.033 mmol), HATU (12.51 mg, 0.033 mmol) and DIPEA (4.25 mg, 0.033 mmol) in anhydrous solvent was stirred at room temperature for 5 min, then compound 26-1 (60 mg, 0.033 mmol) was added. The resulting solution was stirred for another 2 hr at r.t. until LCMS indicated a complete reaction. The reaction solution was purified directly by reverse phase liquid chromatography (40 g C18 column, eluting with 0-70% acetonitrile in water with 0.01% TFA over 15 min) to give compound 37-5 (40 mg, 0.015 mmol, 44.26%) as a white solid. LCMS, ESI m/z=916.4(M/3+H)⁺;

### Step 5

A solution of compound 37-5 (40 mg, 0.015 mmol) in CH₃CN (1.2 mL) and water (0.6 mL) was stirred at r.t., and then Diethyl Amine Anhydrous (0.2 mL, 0.015 mmol) was added. The resulting solution was stirred at r.t. for overnight until LCMS showed that the reaction was completed. Solvent and most of diethyl amine were evaporated, and then the residue was purified by reverse phase liquid chromatography (12 g C18 column, eluting with acetonitrile in water with 0.01% TFA) to yield the expected product 37-6 (20 mg, 0.008 mmol, 54.41%) as a white solid. LCMS, ESI m/z=631.8 (M/4+H)⁺, 842.1 (M/3+H)⁺;2524

### Step 6

A solution of compound 37-6 (20 mg, 0.008 mmol) and DIPEA (5.03 mg, 0.039 mmol) in anhydrous DMF (0.4 mL) was stirred at room temperature for 5 min, and then a solution of 2,5-dioxopyrrolidin-1-yl 6-(2,5-dioxo-2,5-dihydro-1H-pyrrol-1-yl)hexanoate (5.83 mg, 0.019 mmol) in anhydrous DMF (0.1 mL) was added dropwise by syringe over 2 min. The resulting solution was stirred for another 8 hr at r.t. until LCMS indicated all starting amine was consumed and the mass of the desired product was detected. The reaction solution was adjusted to pH 6-7 with diluted formic acid in acetonitrile, and then purified directly by Prep-HPLC (eluting with gradient with 0.01% FA over 20 min) to yield PB037 (4.8 mg, 0.002 mmol) as a white solid. LCMS, m/z=725.8 (M/3+H)⁺;

### Example 8: Preparation of a Drug-Linker Containing a PEG Unit and a Cleavable Linker Attached to Exatecan (PB038)

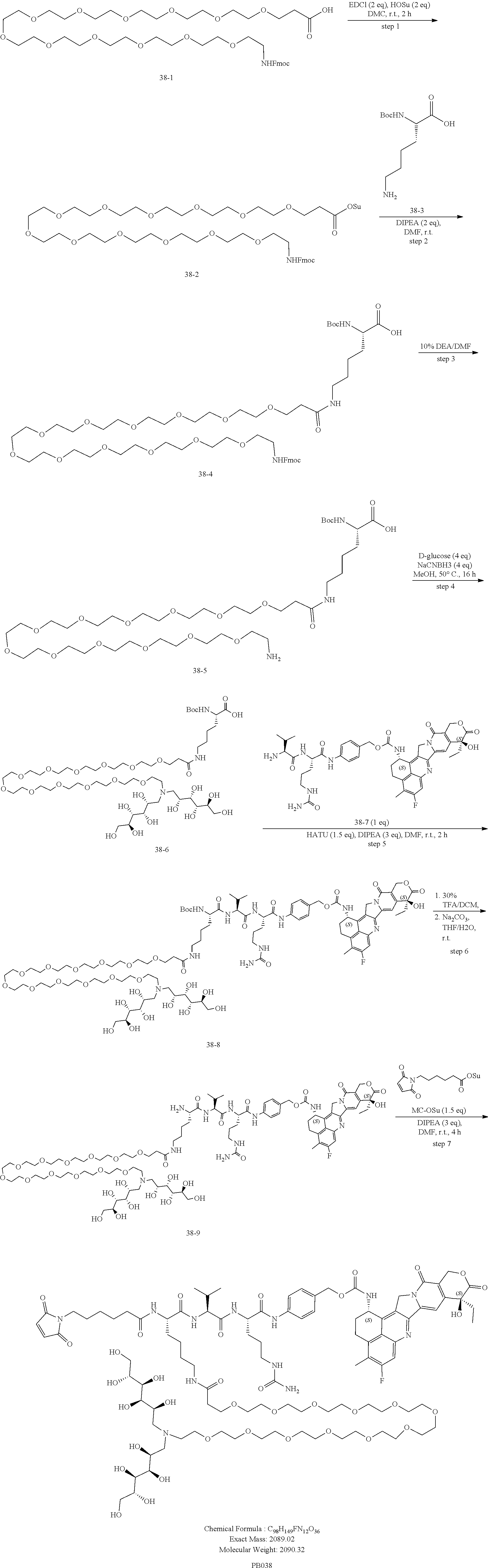

A Drug-Linker (PB038 or LD038) containing a PEG unit and a cleavable linker attached to exatecan was prepared as follows:

### Step 1

A solution of compound 38-1 (650 mg, 0.774 mmol) and N-hydroxylsuccinimide (HOSu) (177.98 mg, 1.548 mmol) in anhydrous DCM (8 mL) was stirred at room temperature, and then EDCl (296.69 mg, 1.548 mmol) was added. The resulting solution was stirred for another 1 hr at r.t. until LCMS indicated that all starting amine was consumed and the desired product was detected. The resulting solution was washed with water, the organic layer was collected, and then the water phase was extracted with DCM (10 mL*2). The combined organic layer was dried over sodium sulfate and filtered, concentrated to dryness to yield compound 38-2 (552 mg, 0.589 mmol, 76.12%) as colorless oil and used as such in the next step (refer to N200897-136). LCMS: m/z=959.4 (M+Na)⁺;

### Step 2

A solution of compound 38-2 (300 mg, 0.357 mmol) and DIPEA (138.22 mg, 1.071 mmol) in anhydrous DMF (2 mL) was stirred at room temperature, and then compound 38-3 (87.97 mg, 0.357 mmol) was added and the starting amine was suspended in the solution. The resulting mixture was kept stirring at r.t. for another 6 hrs. The starting amine dissolved gradually during this period, and the suspension turned into clear light yellow solution. The reaction solution was terminated and purified directly by reverse phase liquid chromatography (40 g C18 column, eluting with 0-100% acetonitrile in water with 0.01% TFA over 15 min) to yield compound 38-4 (260 mg, 0.243 mmol, 68.14%) as pale yellow oil, LCMS ((M-100)/2+H)⁺=484.9;

### Step 3

A solution of compound 38-4 (260 mg, 0.243 mmol) in acetonitrile (1.8 mL) was stirred at r.t. and diethyl amine anhydrous (0.2 mL, 1.941 mmol) was added. The resulting solution was stirred at r.t. for 2 h until the LCMS of the solution showed that most of starting material was consumed. Then the solution was concentrated to dryness and the residue was purified by reverse phase column chromatography (12 g C18 column, eluting with 0-50% acetonitrile in water with 0.01% TFA) to yield the expected fractions of compound 38-5 (170 mg, 0.201 mmol, 82.54%) as pale yellow oil. LCMS, ESI m/z=846.6 (M+H)⁺; Retention time (0.01% TFA)=1.451 min; no UV.

### Step 4

A clear reaction solution of 38-5 (170 mg, 0.201 mmol), D-glucose (217.08 mg, 1.206 mmol) and acetic acid (1.21 mg, 0.020 mmol) in methanol (5 mL) was heated at 50° C. for 30 min, and then NaCNBH₃ (75.98 mg, 1.206 mmol) was added. The resulting solution was stirred at 50° C. under N₂ for 4 hr. Then additional NaCNBH₃ (75.98 mg, 1.206 mmol) and D-glucose (217.08 mg, 1.206 mmol) were added and kept stirring at 50° C. for overnight. After stirring for 20 hr, LCMS indicated the reaction was complete. The solvents were evaporated, and the residue was purified by C18 reversed-phase chromatography to yield the desired product 38-6 (106 mg, 0.090 mmol, 44.92%). LCMS, ESI m/z=537.9 ((M-100)/2+H)⁺;

### Step 5

A solution compound 38-6 (250 mg, 0.213 mmol), HATU (121.45 mg, 0.319 mmol) and DIPEA (82.41 mg, 0.639 mmol) in anhydrous DMF (2 mL) was stirred at room temperature for 5 min, and then compound 38-7 (178.88 mg, 0.213 mmol) was added. The resulting solution was stirred for another 2 hr at r.t. until LCMS indicated a complete reaction. The reaction solution was purified directly by reverse phase liquid chromatography (40 g C18 column, eluting with 0-70% acetonitrile in water with 0.01% TFA over 15 min) to yield compound 38-8 (270 mg, 0.135 mmol, 63.48%) as a white solid. LCMS, ESI m/z=666.6 (M/3+H)⁺, 999.2 (M/2+H)⁺;

### Step 6

A solution of compound 38-8 (120 mg, 0.060 mmol) in TFA (2 mL) was stirred at r.t. for 1 hr. The LCMS of the mixture showed that the reaction was completed, all starting material was consumed, and the desired product (m/z=633=1896/3+H, R.T. 1.501 min) along with the sugar-esterification product (TFA was condensed with hydroxy group in sugar unit, mono-ester with m/z=(1896+96)/2+H=665, R.T. 1.58 min) were formed. The completed reaction solution was condensed to dryness and then redissolved in THF (4 mL) and water (2 mL), and treated with saturated aqueous sodium carbonate solution to adjust the pH to 8-9. The resulting solution was stirred at room temperature for 1 h to achieve complete hydrolysis. The solution was then neutralized with diluted TFA and condensed. The residue was purified by reverse phase liquid chromatography (C18 column, eluting with 0-25% acetonitrile in water with 0.01% TFA for 15 min) to yield the expected product 38-9 (80 mg, 0.042 mmol, 70.19%) as a white solid after lyophilization. LCMS, ESI m/z=633.2 (M/3+H)⁺, 949.2 (M/2+H);

### Step 7

A solution of compound 38-9 (20 mg, 0.011 mmol) and DIPEA (4.08 mg, 0.032 mmol) in anhydrous DMF (1 mL) was stirred at room temperature for 5 min, and then a solution compound 38-10 (4.88 mg, 0.016 mmol) in anhydrous DMF (1 mL) was added dropwise by syringe over 2 min. The resulting solution was stirred for another 4 hr at r.t. until all starting amine was consumed and the mass of the desired product was detected. The resulting solution was neutralized with formic acid to adjust the pH to 6-7. Then the reaction solution was purified by Prep-HPLC (eluting with gradient with 0.01% TFA over 20 min) to yield PB038 (11 mg, 0.005 mmol, 49.91%) as a white solid. LCMS, m/z=697.7 (M/3+H)⁺;

¹HNMR (400 MHz, DMSO-d6): δ 10.03 (s, 1H), 8.19-8.11 (m, 2H), 8.07 (d, J=8.8 Hz, 1H), 7.96 (d, J=7.6 Hz, 1H), 7.82-7.77 (m, 2H), 7.66 (d, J=8.4 Hz, 1H), 7.60 (d, J=8.4 Hz, 2H), 7.36 (d, J=8.0 Hz, 2H), 7.32 (s, 1H), 7.00 (s, 2H), 6.53 (s, 1H), 5.99 (t, J=5.6 Hz, 1H), 5.45-5.43 (m, 6H), 5.30-5.24 (m, 3H), 5.08 (s, 2H), 4.84-4.74 (m, 2H), 4.65-4.49 (m, 4H), 4.45-4.35 (m, 3H), 4.27-4.17 (m, 2H), 4.04-3.95 (m, 2H), 3.80-3.77 (m, 2H), 3.71-3.67 (m, 2H), 3.62-3.55 (m, 9H), 3.53-3.43 (m, 44H), 3.27-3.21 (m, 2H), 3.16-3.07 (m, 2H), 3.07-2.93 (m, 6H), 2.38 (s, 3H), 2.29 (t, J=6.4 Hz, 2H), 2.23-2.13 (m, 2H), 2.13-2.08 (m, 2H), 2.00-1.82 (m, 4H), 1.73-1.54 (m, 4H), 1.54-1.40 (m, 7H), 1.40-1.30 (m, 4H), 1.30-1.14 (m, 5H), 0.90-0.81 (m, 9H) ppm.

### Example 9: Preparation of a Drug-Linker Containing a PEG Unit Attached to a Cleavable Linker and Exatecan (PB0039)

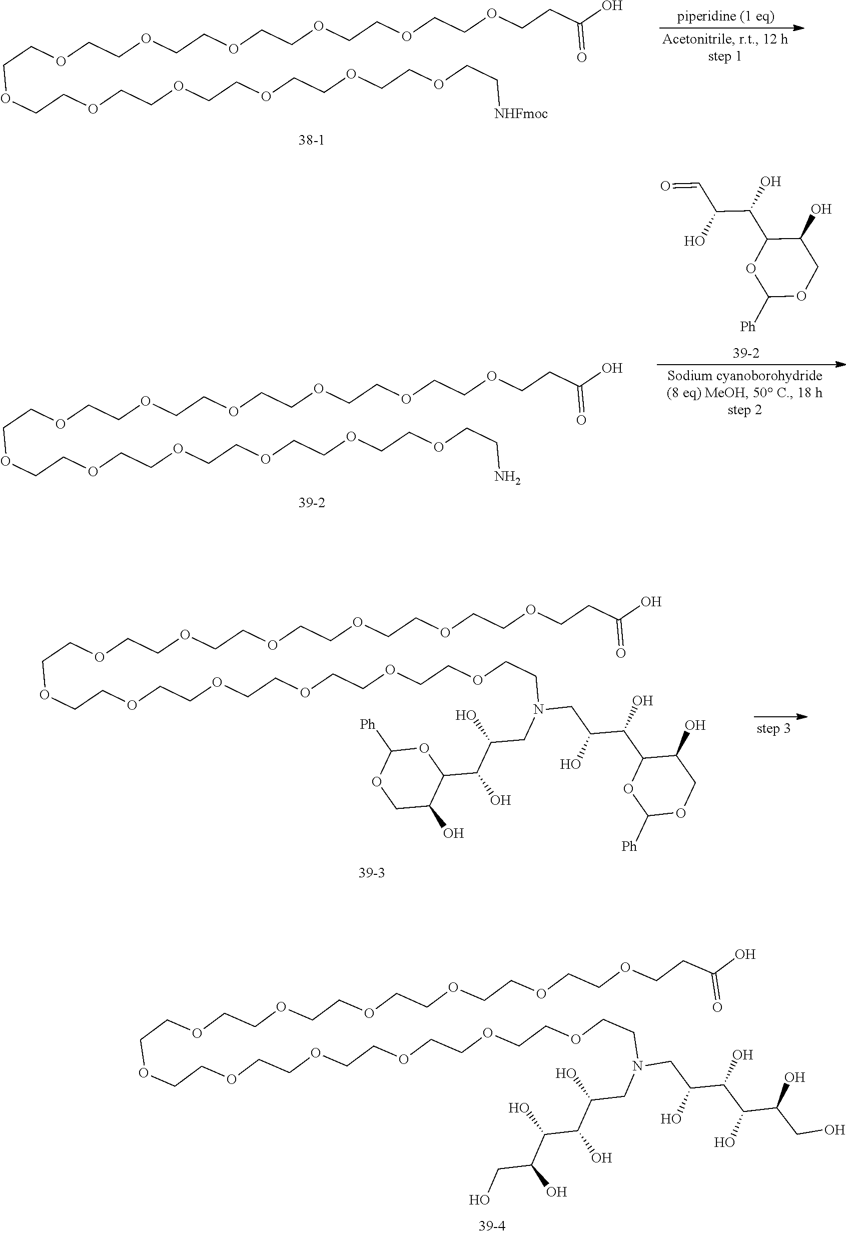

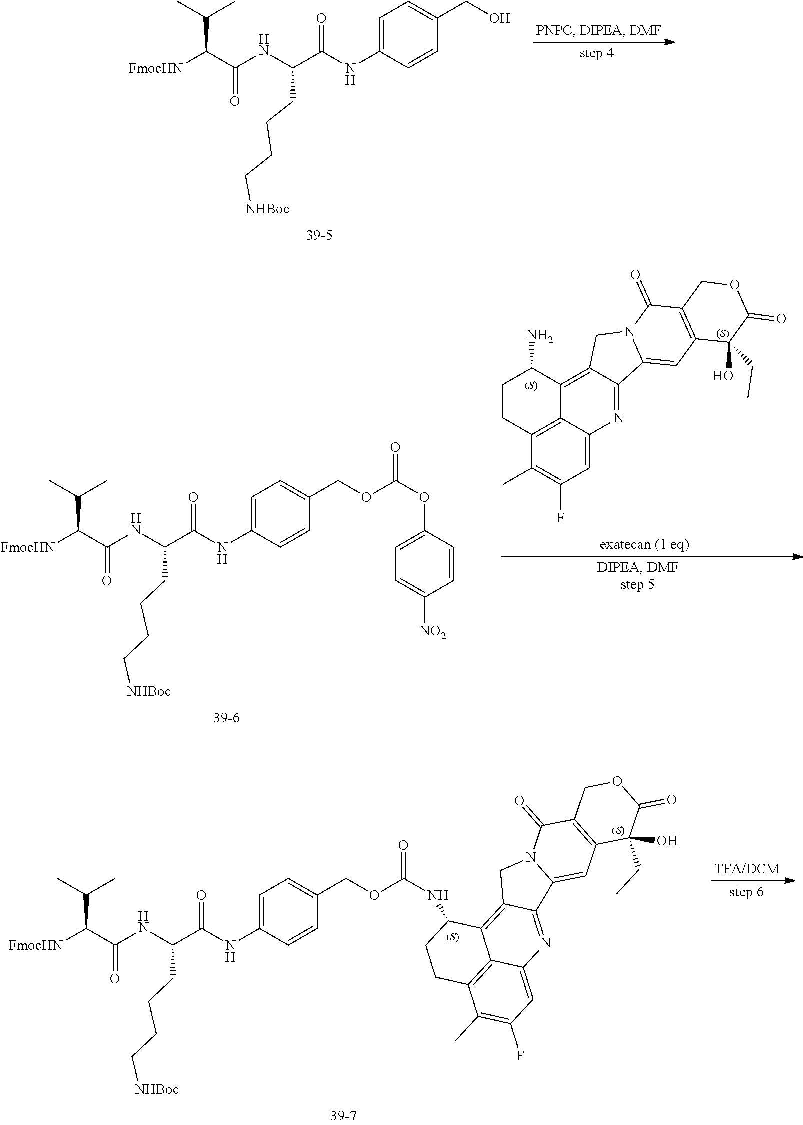

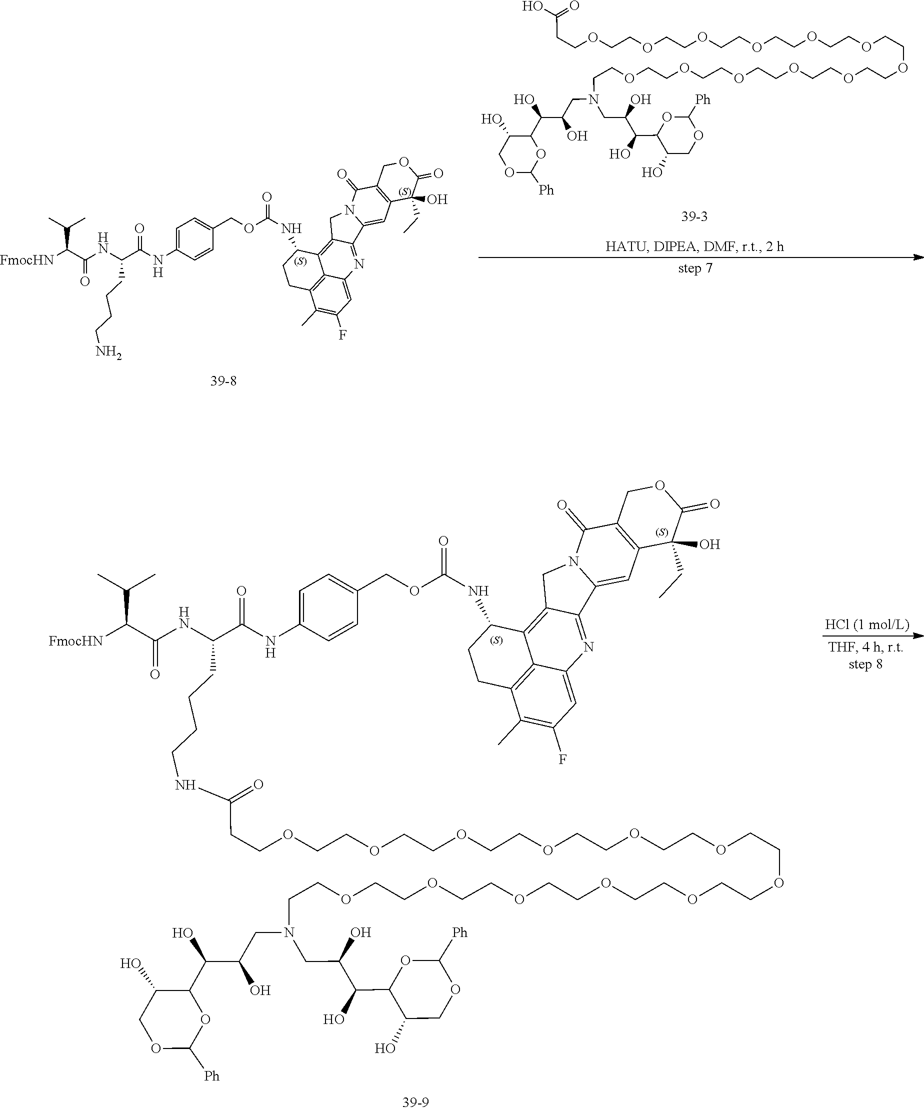

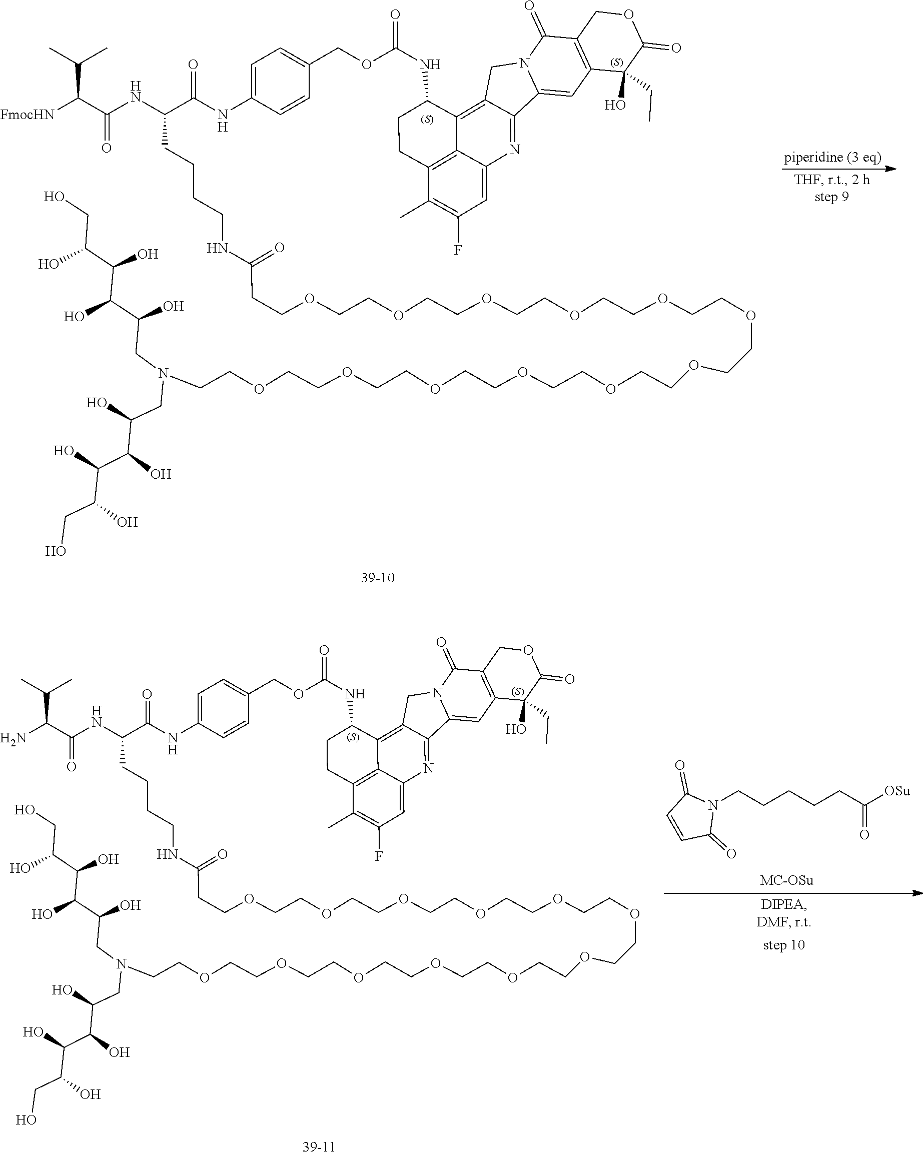

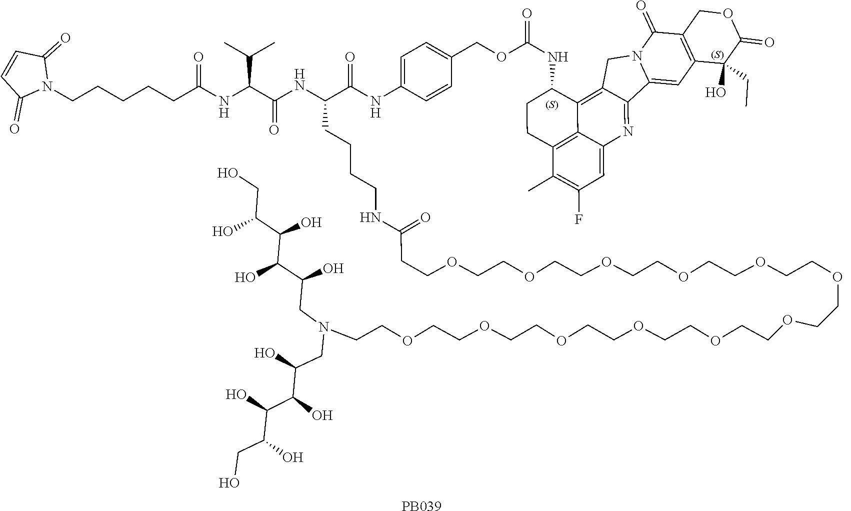

A Drug-Linker containing a PEG unit attached to a cleavable linker and exatecan (PB0039) was prepared as follows:

### Step 1

A solution of compound 38-1 (260 mg, 0.31 mmol) in acetonitrile (3.0 mL) was stirred at r.t. and diethyl amine anhydrous (0.2 mL, 1.941 mmol) was added. The resulting solution was stirred at r.t. for 2 h until LCMS of the solution showed that most of starting material was consumed. Then the solution was concentrated to dryness and the residue was purified by reverse phase column chromatography (12 g C18 column, eluting with 0-50% acetonitrile in water with 0.01% TFA) to yield the expected fractions of compound 39-1 (170 mg, 0.28 mmol) as a pale yellow oil. LCMS, ESI m/z=618.4 (M+H)⁺;

### Step 2

A clear reaction solution of compound 39-1 (170 mg, 0.28 mmol), 39-2 (217.08 mg, 1.206 mmol) and acetic acid (1.21 mg, 0.020 mmol) in methanol (5 mL) was heated at 50° C. for 30 min, and then NaCNBH₃ (75.98 mg, 1.206 mmol) was added. The resulting solution was stirred at 50° C. under N₂ for 4 hr. Then additional NaCNBH₃ (75.98 mg, 1.206 mmol) and compound 39-2 (217.08 mg, 1.206 mmol) was added and kept stirring at 50° C. for overnight. After stirring for 20 hrs, LCMS indicated the reaction was complete. The solvents were evaporated, and the residue was purified by C18 reversed-phase chromatography to yield the desired product 39-3 (265 mg, 0.24 mmol). LCMS, ESI m/z=1122.6 (M+H)⁺.

### Step 3

A mixture of compound 39-3 (265 mg, 0.24 mmol) in 6N HCl/THF aqueous was stirred at rt for 3 hours, until LCMS indicated the reaction was complete. The solvents were neutralized with aqueous NaHCO₃, evaporated, and the residue was purified by C18 reversed-phase chromatography to yield the desired product 39-4 (160 mg, 0.17 mmol). LCMS, ESI m/z=946.5 (M+H)⁺.

### Step 4

A solution of compound 39-5 (3.3 g, 4.905 mmol) and DIPEA (1.90 g, 14.714 mmol) in anhydrous DMF (10 mL) was stirred at room temperature for 5 min, and then PNPC (4.47 g, 14.714 mmol) was added. The resulting bright yellow solution was stirred for another 1.5 hr at r.t. to achieve completion. The resulting solution was quenched with water and the solution was purified directly by reverse phase column chromatography (eluting with 0-100% acetonitrile in water) to yield compound 39-6 (1950 mg, 2.327 mmol) as a yellow solid. LCMS, m/z=860.4 (M+Na)⁺, 738.4 (M-100+H)⁺;

### Step 5

To a solution of compound 39-6 (1.2 g, 1.432 mmol) in DMF (12 mL) was added DIPEA (0.56 g, 4.296 mmol), HOBt (0.10 g, 0.716 mmol) and exatecan (0.69 g, 1.575 mmol). The mixture was stirred at room temperature for 3 h. The resulting solution was purified by reverse phase separation (C18 column, eluting with 0-100% methanol in water with TFA) to afford compound 39-7 (1.2 g, 1.058 mmol), ESI, m/z: 1135.5(M+H)⁺

### Step 6

To the solution of compound 39-7 (620 mg, 0.547 mmol) in DCM (20 mL) was added TFA (2 mL, 0.555 mmol). The mixture was stirred at room temperature for 2 h. The resulting solution was concentrated and purified by reverse phase separation (C18 column, eluting with 0-50% acetonitrile in water with TFA) to afford the product compound 39-8 (409 mg, 0.395 mmol). ESI, m/z: 517.9(M/2+H)⁺.

### Step 7

To a solution of compound 39-3 (600 mg, 0.254 mmol) in DMF (5 mL) was added HATU (97.65 mg, 0.254 mmol) and DIPEA (66.26 mg, 0.502 mmol). The reaction mixture was stirred at r.t. for 10 min. Then the mixture was combined with compound 39-8 (550 mg, 0.232 mmol). The reaction mixture was stirred at r.t. for 2 hours. The resulting solution was concentrated and purified by reverse phase separation (C18 column, eluting with 0-50% acetonitrile in water with TFA) to afford the product 39-9 (500 mg) as a white solid. ESI, m/z: 713.5 (M/2+H)⁺.

### Step 8

To a solution of compound 39-9 (150 mg, 0.070 mmol) in THF (2 mL) was added HCl (2 mol/L, 2 mL). The reaction mixture was stirred at r.t. for 3 hours. The resulting solution was concentrated and purified by reverse phase separation (C18 column, eluting with 0-50% acetonitrile in water with TFA) to afford the product 39-10 (100 mg, 51 mmol) as a white solid. yield 65.69%, purity=90%. ESI, m/z: 655.1 (M/3+H)⁺.

### Step 9

To a solution of compound 39-10 (350 mg, 0.178 mmol) in DMF (2 mL) was added piperidine (76 mg, 0.896 mmol). Then the mixture was stirred at r.t. for 2 hours. The resulting solution was concentrated and purified by reverse phase separation (C18 column, eluting with 0-50% acetonitrile in water with TFA) to afford the product 39-11 (160 mg, 0.092 mmol) as a white solid, yield=51.55%. ESI, m/z: 871.0 (M/2+H)⁺.

### Step 10

To a solution of compound 39-11 (160 mg, 0.092 mmol) in DMF (2 mL) was added DIEA (24 mg, 0.184 mmol) and MC-OSu (56.64 mg, 0.184 mmol). The reaction mixture was stirred at room temperature for 4 hours. The resulting solution was concentrated and purified by reverse phase separation (C18 column, eluting with 0-50% acetonitrile in water with TFA) to afford the product PB039 (62 mg) as a white solid. yield=34.88%. LCMS, m/z=967.2 (M/2+H)⁺;

1HNMR (400 MHz, DMSO-d6): δ 9.96 (s, 1H), 8.04-8.07 (m, 2H), 7.75-7.83 (m, 3H), 7.3 (m, 3H), 6.99 (s, 1H), 6.54 (m, 2H), 5.66 (s, 1H), 5.08-5.28 (m, 3H), 4.51 (m, 2H), 4.14-4.50 (m, 9H), 3.45-3.67 (m, 64H), 1.75-2.75 (m, 19H), 1.73-1.30 (m, 12H), 1.40-1.30 (m, 4H), 1.30-1.14 (m, 5H), 0.90-0.81 (m, 9H) ppm.

### Example 10: Preparation of a Drug-Linker Containing EDTA Attached to a Cleavable Linker and Exatecan (PB040)

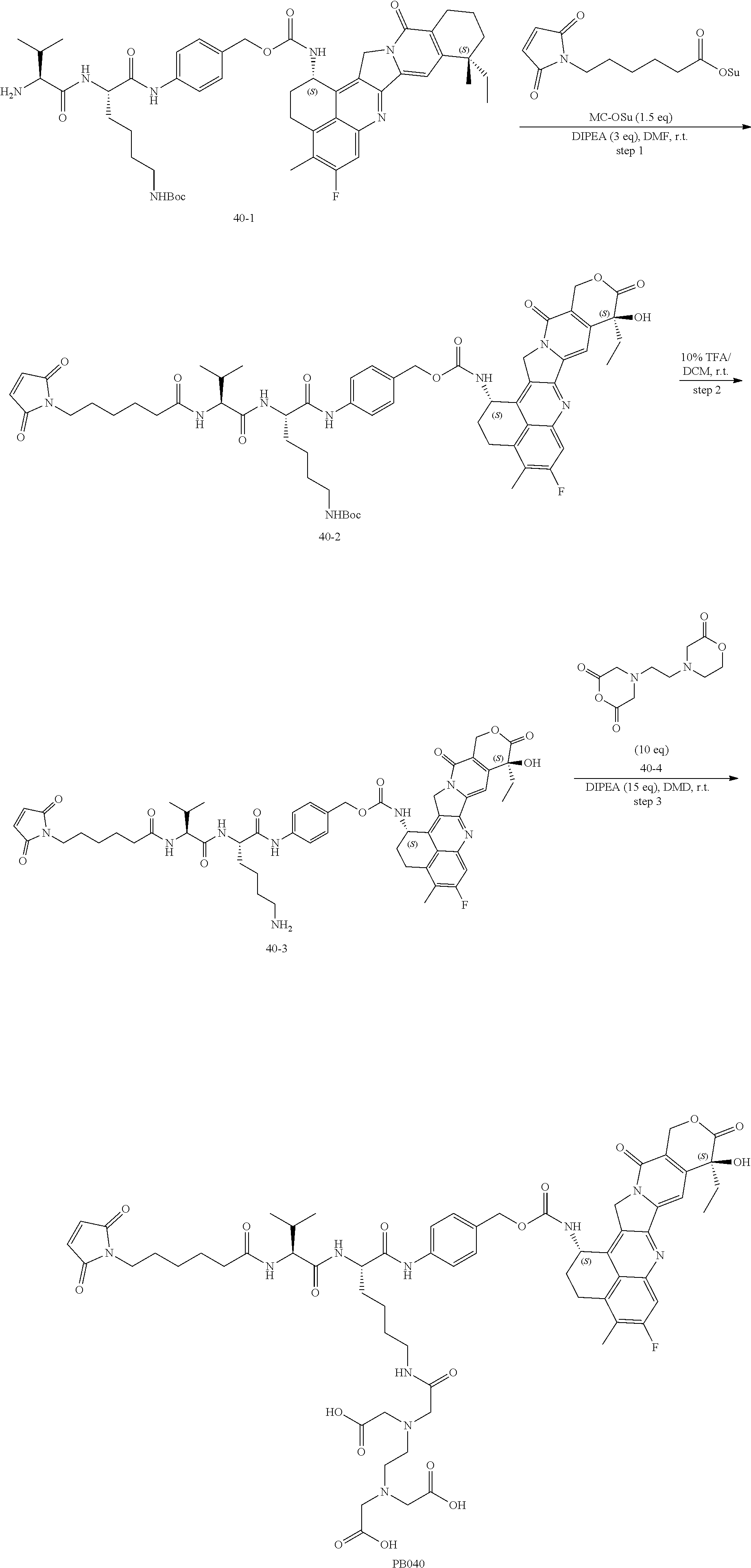

A Drug-Linker containing EDTA attached to a lysine residue of a cleavable linker was prepared as follows:

### Step 1

To a solution of compound 40-1 (31 mg, 0.034 mmol) in DMF (5 mL) was added DIPEA (13.18 mg, 0.102 mmol) and 2,5-dioxopyrrolidin-1-yl 6-(2,5-dioxo-2,5-dihydro-1H-pyrrol-1-yl)hexanoate (20.96 mg, 0.068 mmol). The mixture was stirred at room temperature for 2 h. The resulting solution was adjusted to pH 6 and purified by reverse phase separation (C18 column, eluting with 0-60% acetonitrile in water with TFA) to afford the product 40-2 (25.4 mg, 0.023 mmol, 67.61%), m/z: 1106.4(M+H)⁺.

### Step 2

To a solution of compound 40-2 (83 mg, 0.075 mmol) in DCM (7 mL) was added TFA (0.5 mL, 0.031 mmol). The mixture was stirred at room temperature for 1 h. The resulting solution was concentrated and purified by reverse phase separation (C18 column, eluting with 0-30% acetonitrile in water with TFA) to afford the compound 40-3 (34 mg, 0.034 mmol, 45.04%). m/z: 503.4(M/2+H)⁺.

### Step 3

To a solution of 4-[2-(2,6-dioxomorpholin-4-yl) ethyl] morpholine-2,6-dione (0.037 mL, 0.209 mmol) in DMF (3 mL) was added compound 40-3 (21 mg, 0.021 mmol) and DIPEA (5.40 mg, 0.042 mmol). The mixture was stirred at room temperature for 2 h. The resulting solution was purified by reverse phase separation (C18 column, eluting with 0-45% acetonitrile in water with TFA) to afford the product PB040 (10.8 mg, 0.008 mmol, 40.40%). m/z: 640.4(M/2+H)⁺.

### Example 11: A Drug-Linker Containing Two Sugar Units and a Cleavable Linker Attached to Exatecan (PB041) was Prepared as Follows

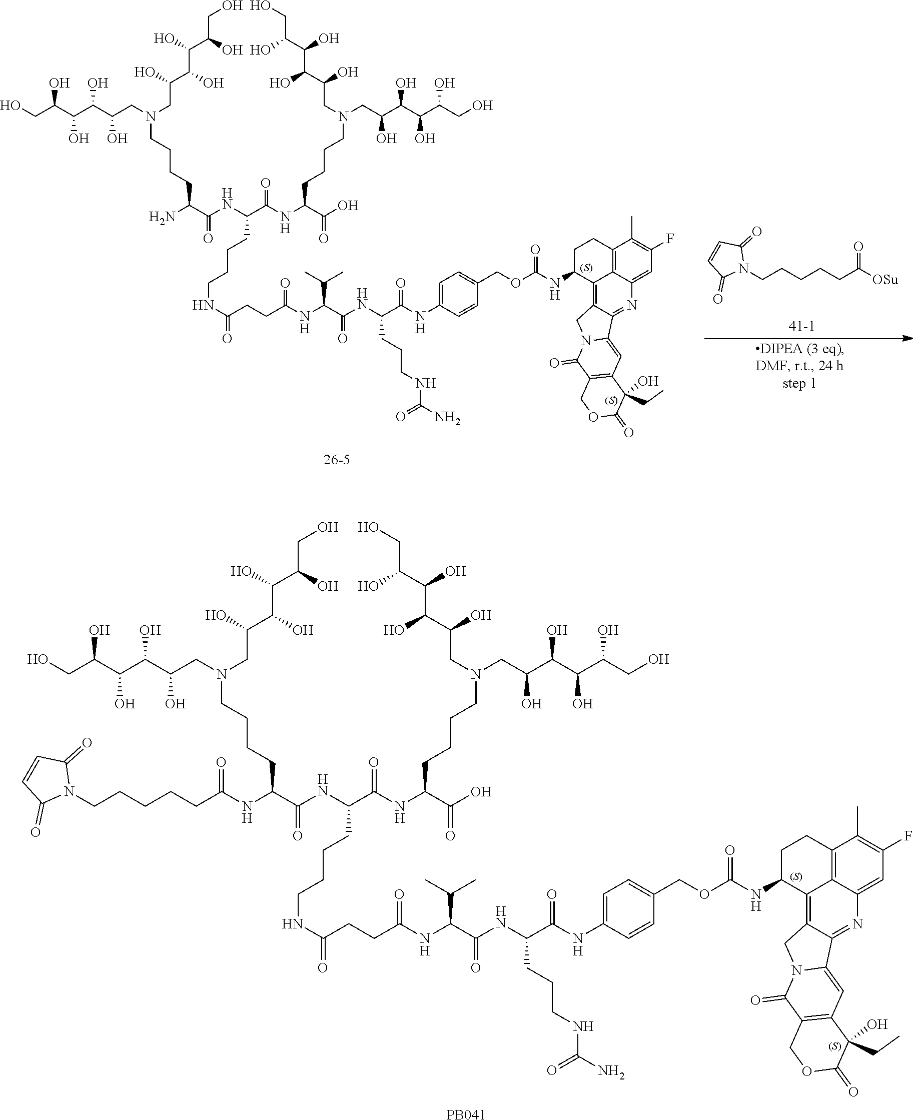

A maleimidylcaproyl Stretcher unit was attached to a Drug-Linker intermediate as follows:

A solution of compound 26-5 (25 mg, 0.013 mmol) and DIPEA (5.03 mg, 0.039 mmol) in anhydrous DMF (0.4 mL) was stirred at room temperature for 5 min, then a solution of Compound 41-1 (2,5-dioxopyrrolidin-1-yl 6-(2,5-dioxo-2,5-dihydro-1H-pyrrol-1-yl)hexanoate) (5.83 mg, 0.019 mmol) in anhydrous DMF (0.1 mL) was added dropwise by syringe over 2 min. The resulting solution was stirred for another 8 hr at r.t. until LCMS indicated that all starting amine was consumed and the mass of desired product was detected. The reaction solution was adjusted to pH 6-7 with diluted formic acid in acetonitrile, then purified directly by Prep-HPLC (eluting with gradient with 0.01% FA over 20 min) to yield PB041 (5.8 mg, 0.003 mmol, 21.14%) as a white solid.

LCMS, m/z=725.8 (M/3+H)⁺;

1H NMR (400 MHz, DMSO-d6): δ 9.72 (s, 1H), 8.31-8.15 (m, 3H), 8.09-8.04 (m, 2H), 7.98-7.96 (m, 1H), 7.96-7.86 (m, 1H), 7.78 (d, J=11.2 Hz, 1H), 7.65 (d, J=7.2 Hz, 2H), 7.36 (d, J=8.0 Hz, 2H), 7.32 (s, 1H), 7.00 (s, 2H), 6.54 (s, 1H), 6.09-6.02 (m, 1H), 5.47-5.45 (m, 4H), 5.30-5.29 (m, 3H), 5.08 (s, 2H), 4.36-4.23 (m, 3H), 4.18-4.13 (m, 1H), 4.13-4.08 (m, 0.5H), 3.92-3.80 (m, 1H), 3.74-3.69 (m, 1H), 3.67-3.56 (m, 10H), 3.05-2.88 (m, 15H), 2.64-2.59 (m, 1H), 2.36-2.33 (m, 12H), 2.24-1.97 (m, 14H), 1.91-1.82 (m, 5H), 1.78-1.61 (m, 14H), 1.53-1.34 (m, 15H), 1.34-1.11 (m, 17H), 0.89-0.86 (m, 11H) ppm. 19F NMR (400 MHz, DMSO-d6): δ-111 ppm.

### Example 12: Preparation of a Drug-Linker Containing a PEG Unit and a Cleavable Linker Attached to Exatecan (PB050) was Prepared as Follows

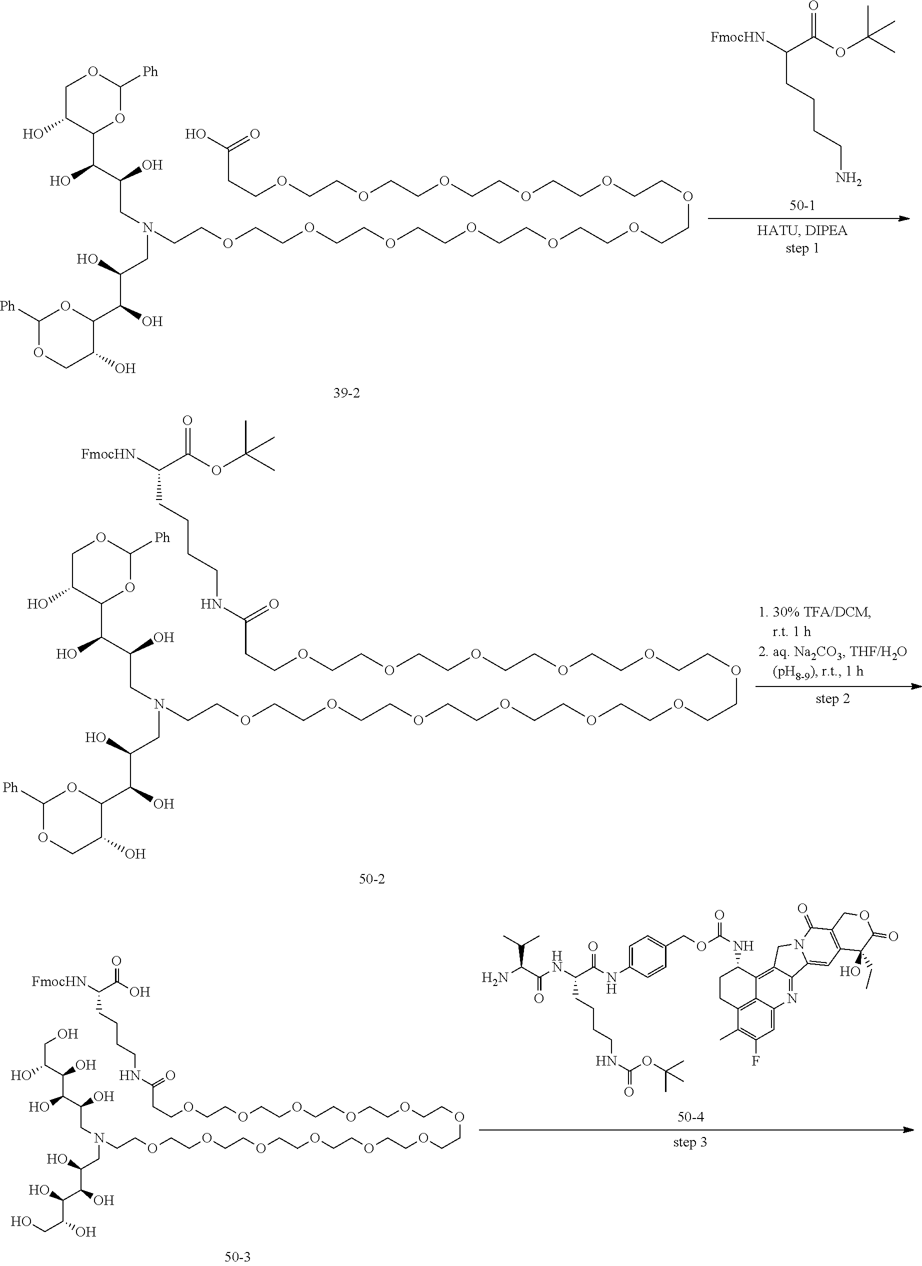

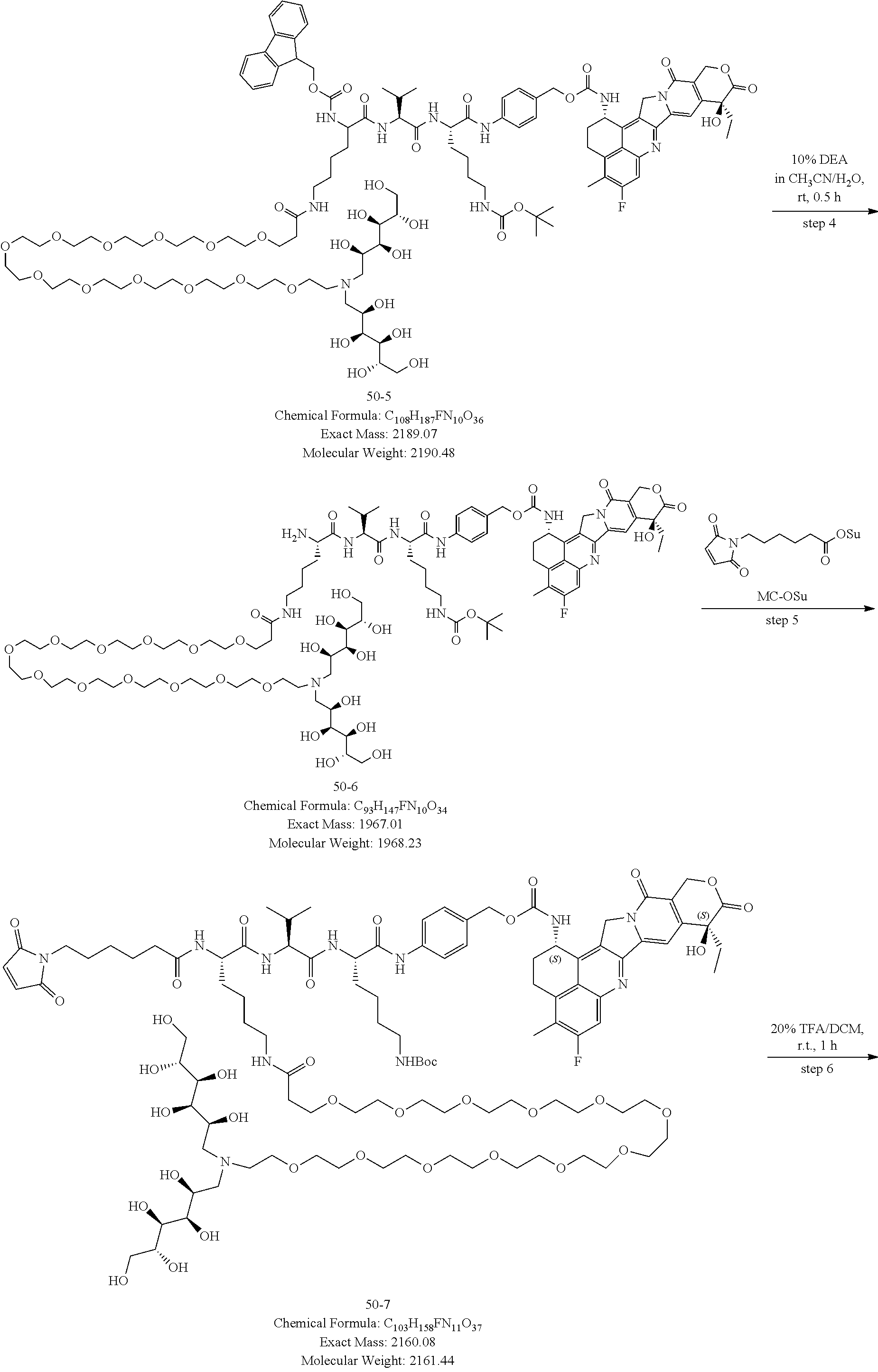

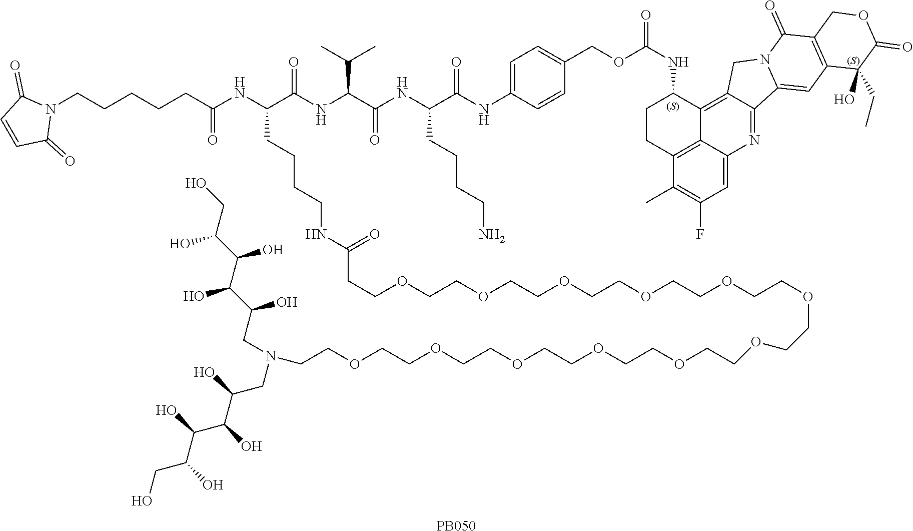

A Drug-Linker containing a PEG linker and a cleavable linker attached to exatecan (PB050) was prepared as follows:

### Step 1

A solution of compound 50-1 (56.74 mg, 0.134 mmol), HATU (60.99 mg, 0.160 mmol) and DIPEA (51.73 mg, 0.401 mmol) in anhydrous DMF (1.5 mL) was stirred at room temperature for 5 min, then compound 39-2 (150 mg, 0.134 mmol) was added. The resulting solution was stirred for another1 hr at r.t. until LCMS indicated the reaction was complete. The reaction solution was purified directly by reverse phase liquid chromatography (40 g C18 column, eluting with 0-70% acetonitrile in water with 0.01% TFA over 15 min) to yield compound 50-2 (130 mg, 0.085 mmol, yield=63.62%) as a white solid. LCMS, ESI m/z=763.8 (M/2+H)⁺

### Step 2

A solution of compound 50-2 (130 mg, 0.085 mmol) in DCM (0.7 mL) was stirred at room temperature for 5 min, and then TFA (0.3 mL, 4.039 mmol) was added. The resulting solution was stirred for another 2 hr at r.t. until LCMS indicated the reaction was completed. The reaction solution was concentrated to dryness under vacuo, and the residue was then purified directly by reverse phase column chromatography (eluting with gradient with 0.01% FA over 20 min) to yield compound 50-3 (60 mg, 0.046 mmol, 54.43%) as a white solid. LCMS, m/z=725.8 (M/3+H)⁺;

1H NMR (400 MHz, DMSO-d6): δ 9.72 (s, 1H), 8.31-8.15 (m, 3H), 8.09-8.04 (m, 2H), 7.98-7.96 (m, 1H), 7.96-7.86 (m, 1H), 7.78 (d, J=11.2 Hz, 1H), 7.65 (d, J=7.2 Hz, 2H), 7.36 (d, J=8.0 Hz, 2H), 7.32 (s, 1H), 7.00 (s, 2H), 6.54 (s, 1H), 6.09-6.02 (m, 1H), 5.47-5.45 (m, 4H), 5.30-5.29 (m, 3H), 5.08 (s, 2H), 4.36-4.23 (m, 3H), 4.18-4.13 (m, 1H), 4.13-4.08 (m, 0.5H), 3.92-3.80 (m, 1H), 3.74-3.69 (m, 1H), 3.67-3.56 (m, 10H), 3.05-2.88 (m, 15H), 2.64-2.59 (m, 1H), 2.36-2.33 (m, 12H), 2.24-1.97 (m, 14H), 1.91-1.82 (m, 5H), 1.78-1.61 (m, 14H), 1.53-1.34 (m, 15H), 1.34-1.11 (m, 17H), 0.89-0.86 (m, 11H) ppm.

### Step 3

A solution of compound 50-3 (59 mg, 0.046 mmol), HATU (20.76 mg, 0.055 mmol) and DIPEA (17.61 mg, 0.137 mmol) in anhydrous DMF (1 mL) was stirred at room temperature for 5 min, and then compound 50-4 (41.51 mg, 0.046 mmol) was added. The resulting solution was stirred for another1 hr at r.t. until LCMS indicated the reaction was complete. The reaction solution was purified directly by reverse phase liquid chromatography (40 g C18 column, eluting with 0-50% acetonitrile in water with 0.01% TFA over 15 min) to yield compound 50-5 (40 mg, 0.018 mmol, 40.12%) as a white solid. LCMS, m/z=697.6 ((M-100)/2+H)⁺; 1H NMR (400 MHz, DMSO-d6) δ 10.02 (s, 1H), 8.20-8.14 (m, 1H), 8.14-7.98 (m, 2H), 7.88 (d, J=7.6 Hz, 2H), 7.82-7.70 (m, 5H), 7.64-7.52 (m, 4H), 7.43-7.31 (m, 7H), 6.78-6.72 (m, 1H), 6.53 (s, 1H), 5.45-5.44 (m, 4H), 5.34-5.23 (m, 3H), 5.08 (s, 2H), 4.82-4.77 (m, 2H), 4.62-4.36 (m, 6H), 4.36-4.16 (m, 6H), 4.05-3.95 (m, 3H), 3.81-3.73 (m, 2H), 3.73-3.66 (m, 2H), 3.62-3.56 (m, 9H), 3.56-3.34 (m, 50H), 3.05-3.01 (m, 2H), 2.95-2.84 (m, 2H), 2.38 (s, 3H), 2.29 (t, J=6.0 Hz, 2H), 2.19-2.10 (m, 2H), 2.01-1.93 (m, 1H), 1.90-1.83 (m, 2H), 1.71-1.48 (m, 4H), 1.40-1.23 (m, 18H), 0.90-0.81 (m, 9H) ppm. One proton signal of TFA in included. 19F NMR (400 MHz, DMSO-d6), b TFA at −73 ppm, Ar-F at −111 ppm

### Step 4

A solution of compound 50-5 (40 mg, 0.018 mmol) in CH₃CN (2 mL) and water (1 mL) was stirred at r.t. and diethyl amine anhydrous (0.002 mL, 0.018 mmol) was added. The resulting solution was stirred at r.t. for 2 h until LCMS showed a complete reaction. All the solvent was evaporated to yield a crude solid, which was suspended in more acetonitrile and evaporated again to remove diethyl amine completely. Then the residue was dissolved in acetonitrile and water, acidified with formic acid to pH 2-3 and let stand for 1h; LCMS indicated all the lactone ring was closed back. Then the solution was lyophilized overnight to yield product compound 50-6 (36 mg, 0.018 mmol, 100.17%) as a pale yellow solid. This compound was used as such for the next step without any purification. LCMS: (crude, treated with formic acid, m/z=656.9 (M/3+H)⁺, 984.6 (M/2+H)⁺;

### Step 5

A solution of compound 50-6 (35.43 mg, 0.018 mmol) and DIPEA (6.97 mg, 0.054 mmol) in anhydrous DMF (0.8 mL) was stirred at room temperature for 5 min, then a solution of 2,5-dioxopyrrolidin-1-yl 6-(2,5-dioxo-2,5-dihydro-1H-pyrrol-1-yl) hexanoate (8.32 mg, 0.027 mmol) in anhydrous DMF (0.2 mL) was added dropwise by syringe. The resulting solution was stirred for another 1 hr at r.t. until LCMS indicated all starting amine was consumed and the mass of the desired product was detected. The resulting solution was acidified to pH 3-4 with formic acid and then purified directly by reverse phase flash chromatography (40 g C18 column, eluting with 0-70% acetonitrile in water with 0.01% T FA over 20 min) to give desired fractions, which was lyophilized to yield compound 50-7 (28 mg, 0.013 mmol, 71.96%) as a white solid. LCMS, m/z=687.9 ((M-100)/3+H)⁺;

### Step 6

To a solution of compound 50-7 (25 mg, 0.012 mmol) in DCM (4 mL) was added TFA (1 mL, 13.463 mmol) and then stirred at r.t. for 2 h until LCMS showed that the reaction was completed. TFA and DCM were evaporated under reduced pressure, then the residue was purified by Prep-HPLC (eluting with 0-100% acetonitrile in water with 0.01% TFA over 15 min) and the expected fractions were lyophilized to yield product PB050 as a white solid. LCMS, ESI m/z=516.3 (M/4+H)⁺, 687.9 (M/3+H); 1H NMR (400 MHz, DMSO-d6): δ 10.06 (s, 1H), 8.16 (d, J=7.2 Hz, 1H), 8.07 (d, J=7.6 Hz, 1H), 7.99 (d, J=8.0 Hz, 1H), 7.84-7.80 (m, 2H), 7.77 (brs, 3H), 7.66-7.59 (m, 3H), 7.37 (d, J=8.8 Hz, 2H), 7.32 (s, 1H), 7.00 (s, 2H), 6.54 (s, 1H), 6.24 (s, 4H), 5.45 (s, 2H), 5.35-5.24 (m, 3H), 5.08 (s, 2H), 4.86-4.43 (m, 5H), 4.43-4.32 (m, 1H), 4.25-4.12 (m, 2H), 4.02-3.94 (m, 2H), 3.79-3.73 (m, 2H), 3.69-3.67 (m, 2H), 3.59-3.58 (m, 1H), 3.57-3.55 (m, 9H), 3.55-3.47 (m, 52H), 3.04-2.95 (m, 2H), 2.81-2.73 (m, 2H), 2.38 (s, 3H), 2.29 (t, J=6.4 Hz, 2H), 2.23-2.02 (m, 5H), 2.03-1.93 (m, 2H), 1.93-1.83 (m, 3H), 1.76-1.67 (m, 1H), 1.59-1.43 (m, 10H), 1.39-1.24 (m, 6H), 1.24-1.15 (m, 5H), 0.90-0.81 (m, 9H) ppm. 19F NMR (400 MHz, DMSO-d6): −111 ppm

### Example 13: Preparation of a Drug-Linker Containing a PEG Unit and a Cleavable Linker Attached to Exatecan (PB082)

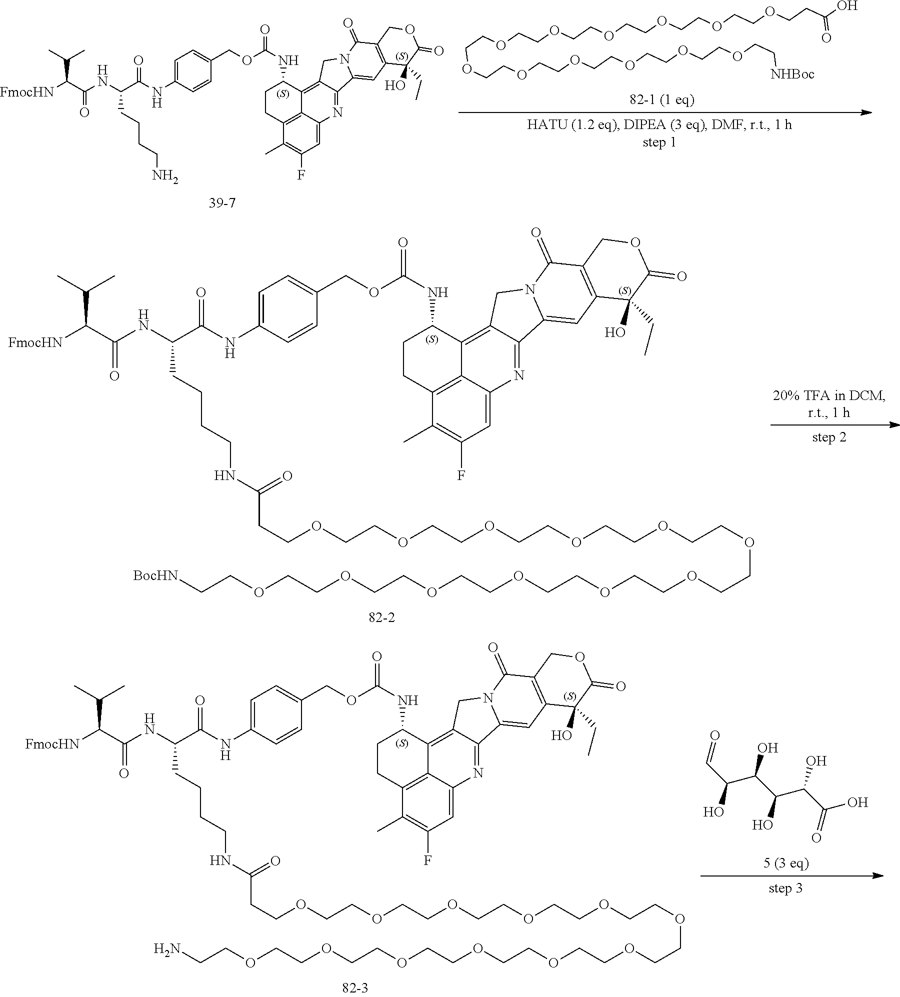

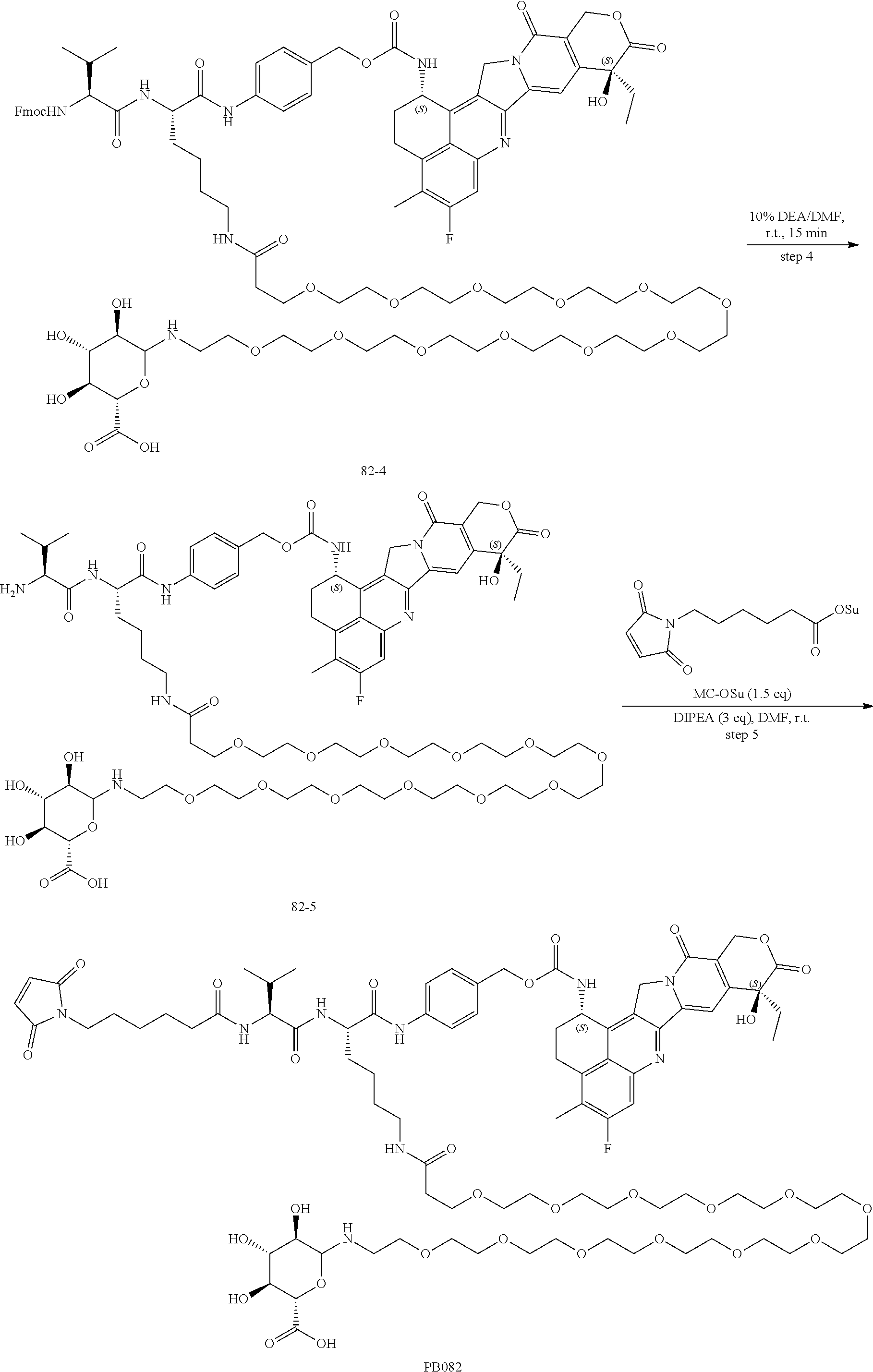

A Drug-Linker (PB082) containing a PEG unit and a cleavable linker attached to exatecan was prepared as follows:

### Step 1

A solution of compound 82-1 (173.53 mg, 0.242 mmol), HATU (110.30 mg, 0.290 mmol) and DIPEA (93.55 mg, 0.725 mmol) in anhydrous DMF (3 mL) was stirred at room temperature for 5 min, then compound 39-7 (250 mg, 0.242 mmol) was added. The resulting solution was stirred for another 2 hr at r.t. until LCMS indicatedcomplete reaction. The reaction solution was purified directly by reverse phase liquid chromatography (40 g C18 column, eluting with 0-70% acetonitrile in water with 0.01% TFA over 15 min) to yield compound 82-2 (270 mg, 0.156 mmol, 64.41%) as a pale yellow solid.

### Step 2

A solution of compound 82-2 (870 mg, 0.502 mmol) in DCM (4 mL) was stirred at room temperature, then TFA (1 mL, 13.463 mmol) was added, and the light yellow solution was stirred for 1h. until LCMS of the solution showed that the deprotection was completed. The solvent and TFA were evaporated, then the residue was purified directly by reverse phase liquid chromatography (40 g C18 column, eluting with 0-20% acetonitrile in water with 0.01% TFA for 10 min) to yield the expected fractions, which were freeze-dried to yield compound 82-3 (650 mg, 0.398 mmol, 79.29%) as a white solid. LCMS, m/z=545.5 (M/3+H)⁺, 817.6 (M/2+H)⁺;

### Step 3

A mixture of compound 82-3 (220 mg, 0.213 mmol) and (2S,3S,4S,5R)-2,3,4,5-tetrahydroxy-6-oxohexanoic acid (123.90 mg, 0.638 mmol) in methanol (6 mL) was heated at 50° C. for 8 hrs to achieve complete conversion. Then the suspension was concentrated to remove methanol, the residue was dissolved in DMF and purified by reverse phase column chromatography (40 g C18 column, eluting with 0-50% acetonitrile in water with 10 mM ammonium bicarbonate over 15 min) to collect the desired fractions, which were freeze-dried to yield product 82-4 as a white solid. LCMS, m/z=604.2(M/3+H)⁺, 905.6 (M/2+H)⁺,

### Step 4

A solution of compound 82-4 (100 mg, 0.055 mmol) in DMF (0.9 mL) was stirred at r.t. and diethyl amine anhydrous (0.1 mL, 0.971 mmol) was added. The resulting solution was stirred at r.t. for 15 min. The LCMS of the reaction mixture showed that the reaction was completed. Most of the diethyl amine and other volatiles were evaporated, then the residue was acidified to pH 3-4 with formic acid, and then purified by reverse phase flash chromatography (0-40% acetonitrile in water with 0.01% TFA) to yield expected compound 82-5 (35 mg, 0.022 mmol, 39.90%) as a white solid. LCMS, m/z=794.5 (M/2+H)⁺, 530.2 (M/2+H)⁺;

### Step 5

A solution of compound 82-5 (150 mg, 0.083 mmol) and DIPEA (8.53 mg, 0.066 mmol) in anhydrous DMF (1 mL) was stirred at room temperature for 5 min, then a solution MC-OSu (10.20 mg, 0.033 mmol) in anhydrous DMF (1 mL) was added dropwise by syringe. The resulting solution was stirred for another 6 hr at r.t. until the desired product was detected as major new peak, along with little byproduct The resulting solution was acidified by formic acid to pH3-4, then purified directly by Prep-HPLC (eluting with gradient with 0.01% TFA over 20 min) to yieldproduct PB082 (10 mg, 0.006 mmol, 25.52%) as a white solid. LCMS, m/z=594.5(M/3+H)⁺,891.3(M/2+H)⁺891.3(M/2+H)⁺

1H NMR: (500 MHz, DMSO-d6) δ 9.96 (s, 1H), 8.07-8.02 (m, 2H), 7.83-7.81 (m, 2H), 7.77 (d, J=10.5 Hz, 1H), 7.59 (d, J=8.0 Hz, 2H), 7.36 (d, J=8.5 Hz, 2H), 7.31 (s, 1H), 6.99 (s, 2H), 6.67 (s, 1H), 6.53 (s, 1H), 5.84-5.79 (m, 1H), 5.45 (s, 2H), 5.33-5.23 (m, 3H), 5.08 (s, 2H), 4.35-4.24 (m, 2H), 4.18-4.13 (m, 1H), 4.10-4.04 (m, 1H), 3.92-3.91 (m, 1H), 3.73-3.67 (m, 2H), 3.58-3.54 (m, 6H), 3.52-3.45 (m, 44H), 3.21-3.09 (m, 6H), 3.03-2.99 (m, 2H), 2.38 (s, 3H), 2.28 (t, J=6.5 Hz, 2H), 2.21-2.08 (m, 4H), 2.00-1.90 (m, 1H), 1.90-1.82 (m, 2H), 1.74-1.65 (m, 1H), 1.65-1.57 (m, 1H), 1.52-1.45 (m, 4H), 1.45-1.27 (m, 5H), 1.24-1.15 (m, 2H), 0.90-0.81 (m, 9H) ppm.

### Example 14: Preparation of a Drug-Linker Containing a PEG Unit and a Cleavable Linker Attached to Exatecan (PB083)

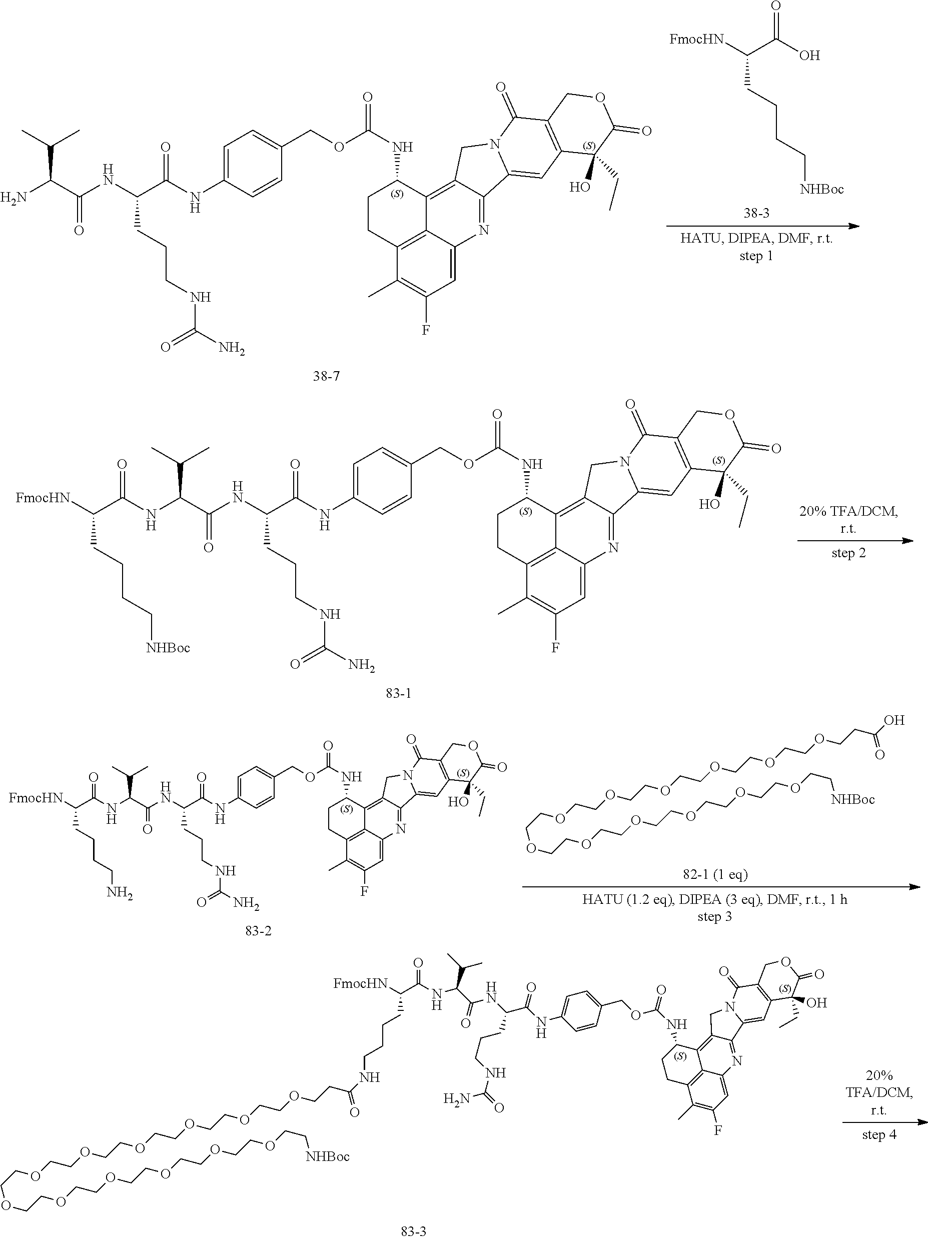

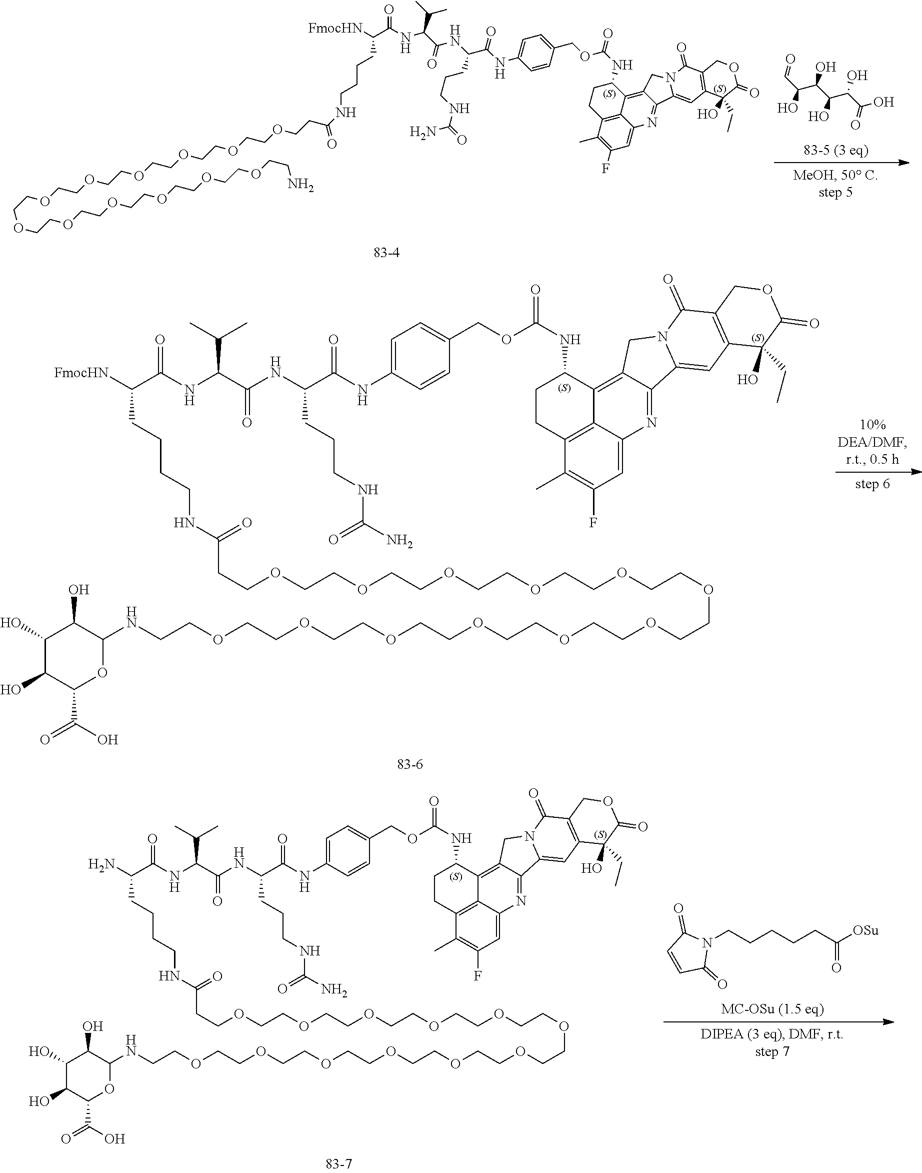

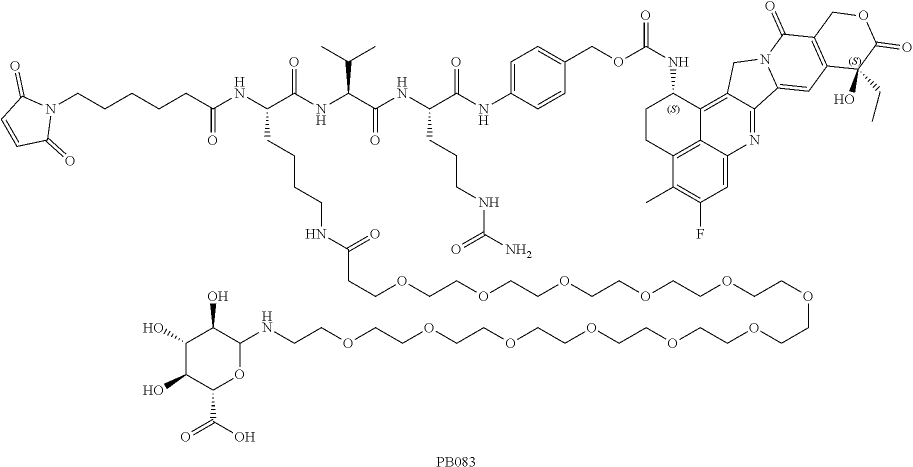

A Drug-Linker containing a PEG unit and a cleavable linker attached to exatecan (PB083) was prepared as follows:

### Step 1

A solution of compound 38-3 (0.190 mL, 0.499 mmol), HATU (284.86 mg, 0.749 mmol) and DIPEA (193.29 mg, 1.498 mmol) in anhydrous DMF (3 mL) was stirred at room temperature for 5 min, and then a solution of compound 38-7 (420 mg, 0.499 mmol) in anhydrous DMF (2 mL) was added. The resulting solution was stirred for another 1.5 hr at r.t. until LCMS indicated the reaction was complete. The reaction solution was purified directly by reverse phase liquid chromatography (40 g C18 column, eluting with 0-100% acetonitrile in water with 0.01% TFA over 15 min then 100% methanol for 5 min) to yield compound 83-1 (330 mg, 0.256 mmol, 51.16%) as a pale yellow solid. LCMS, m/z=596.4 ((M-100)/2+H.

1H NMR (400 MHz, DMSO-d6) δ 10.05 (s, 1H), 8.14 (d, J=7.6 Hz, 1H), 8.06 (d, J=9.2 Hz, 1H), 7.89 (d, J=7.6 Hz, 2H), 7.78 (d, J=11.2 Hz, 1H), 7.73-7.71 (m, 3H), 7.60 (d, J=8.4 Hz, 2H), 7.53 (d, J=8.4 Hz, 1H), 7.43-7.31 (m, 8H), 6.81-6.73 (m, 1H), 6.03-5.93 (m, 1H), 5.54-5.37 (m, 3H), 5.37-5.24 (m, 3H), 5.08 (s, 2H), 4.44-4.34 (m, 1H), 4.34-4.23 (m, 4H), 4.06-3.97 (m, 1H), 3.29-3.13 (m, 1H), 3.13-2.98 (m, 2H), 2.98-2.81 (m, 3H), 2.38 (s, 3H), 2.26-2.17 (m, 2H), 2.09-1.96 (m, 1H), 1.96-1.73 (m, 2H), 1.67-1.54 (m, 3H), 1.45-1.23 (m, 17H), 0.89-0.81 (m, 9H) ppm.

### Step 2

To a solution of compound 83-1 (330 mg, 0.256 mmol) in DCM (4 mL) was added TFA (1 mL, 6.228 mmol), and the solution was stirred at r.t. for 1h until LCMS of the solution showed that the reaction was completed. Solvents were evaporated under reduced pressure, then the residue was purified by reverse phase liquid chromatography (40 g C18 column, eluting with 0-40% acetonitrile in water with 0.01% TFA for 10 min) to yield product compound 83-2 (300 mg, 0.252 mmol, 98.55%) as a pale yellow solid. LCMS, m/z=596.3(M/2+H)⁺;

### Step 3

A solution of compound 82-1 (180.77 mg, 0.252 mmol), HATU (114.90 mg, 0.302 mmol) and DIPEA (97.45 mg, 0.755 mmol) in anhydrous DMF (1.5 mL) was stirred at room temperature for 5 min, then a solution compound 83-2 (300 mg, 0.252 mmol) in anhydrous DMF (0.5 mL) was added. The resulting solution was stirred for another 2 hrs at r.t. until LCMS indicated a complete reaction. The reaction solution was purified directly by reverse phase liquid chromatography (40 g C18 column, eluting with 0-80% acetonitrile in water with 0.01% TFA over 15 min) to yield compound 83-3 (270 mg, 0.143 mmol, 56.70%) as a white solid. LCMS: m/z=946.1 (M/2+H)⁺;

### Step 4

A solution compound 83-3 (450 mg, 0.238 mmol) in DCM (8 mL) was stirred at r.t., then TFA (2 mL, 26.925 mmol) was added to the solution. The resulting yellow solution was stirred for 1 h to achieve complete deprotection. The completed solution was concentrated to remove DCM and TFA, then the brown residue was purified by reverse phase liquid chromatography (40 g C18 column, eluting with 0-50% acetonitrile in water with 0.01% TFA for 15 min) to yield expected product compound 83-4 as a white solid. LCMS, m/z=597.8 (M/3+H)⁺, 896.6 (M/2+H)⁺;

### Step 5

A reaction mixture compound 83-4 (250 mg, 0.140 mmol), compound 83-5 (81.30 mg, 0.419 mmol), HOAc (0.025 mL, 0.140 mmol) in methanol (5 mL) was stirred at 50° C. under N2 for 18 hrs. Then LCMS indicated most of compound 83-5 was consumed and the desired product was detected. The solvents were evaporated, and the residue was purified by C18 reversed-phase chromatography to yield the desired compound 83-6 (100 mg, 0.051 mmol, 36.42%). LCMS, m/z=656.3 (M/3+H)⁺, 983.8 (M/2+H)⁺;

### Step 6

The solution of compound 83-6 (100 mg, 0.051 mmol) in DMF (0.9 mL) was stirred at r.t. and diethyl amine anhydrous (0.1 mL, 0.971 mmol) was added. The resulting solution was stirred at r.t. for 15 min until LCMS showed the reaction was complete and the mass of the desired product was detected. Then the crude product was purified by reverse phase liquid chromatography (40 g C18 column, eluting with 0-50% acetonitrile in water with 0.01% TFA for 15 min) to yield expected product compound 83-7 as a white solid LCMS, m/z=582.5 (M/3+H)⁺;

### Step 7

A solution of compound 83-7 (50 mg, 0.029 mmol) and DIPEA (11.09 mg, 0.086 mmol) in anhydrous DMF (2 mL) was stirred at room temperature for 5 min, then a solution of MC-OSu (13.25 mg, 0.043 mmol) in anhydrous DMF (2 mL) was added dropwise by syringe. The resulting solution was stirred for another 6 hr at r.t. until LCMS indicated all starting amine was consumed and the desired product was detected. The resulting solution was acidified by adding formic acid to pH3-4, then purified directly by Prep-HPLC (eluting with gradient with 0.01% TFA over 20 min) to yield product PB083 (21 mg, 0.011 mmol, 37.81%) as a white solid. LCMS, m/z=485.7 (M/4+H)⁺, 646.9 (M/3+H)⁺

1H NMR: b 10.04 (s, 1H), 8.14-8.05 (m, 2H), 7.96 (d, J=7.6 Hz, 1H), 7.83-7.76 (m, 2H), 7.67 (d, J=7.6 Hz, 1H), 7.60 (d, J=8.4 Hz, 2H), 7.36 (d, J=8.4 Hz, 2H), 7.31 (s, 1H), 6.99 (s, 2H), 6.53 (brs, 1H), 6.05-5.97 (m, 1H), 5.45-5.43 (m, 4H), 5.34-5.24 (m, 3H), 5.08 (s, 2H), 4.43-4.32 (m, 1H), 4.27-4.17 (m, 3H), 4.05-4.02 (m, 1H), 3.87-3.80 (m, 1H), 3.71-3.64 (m, 2H), 3.57-3.47 (m, 53H), 3.16-3.06 (m, 2H), 3.06-2.92 (m, 6H), 2.38 (s, 3H), 2.29 (t, J=6.4 Hz, 2H), 2.24-2.11 (m, 4H), 2.03-1.83 (m, 4H), 1.74-1.56 (m, 3H), 1.50-1.35 (m, 9H), 1.35-1.14 (m, 5H), 0.89-0.81 (m, 9H) ppm.

### Example 15: Preparation of a Drug-Linker Containing a PEG Unit and a Cleavable Linker Attached to MMAE (PB084)

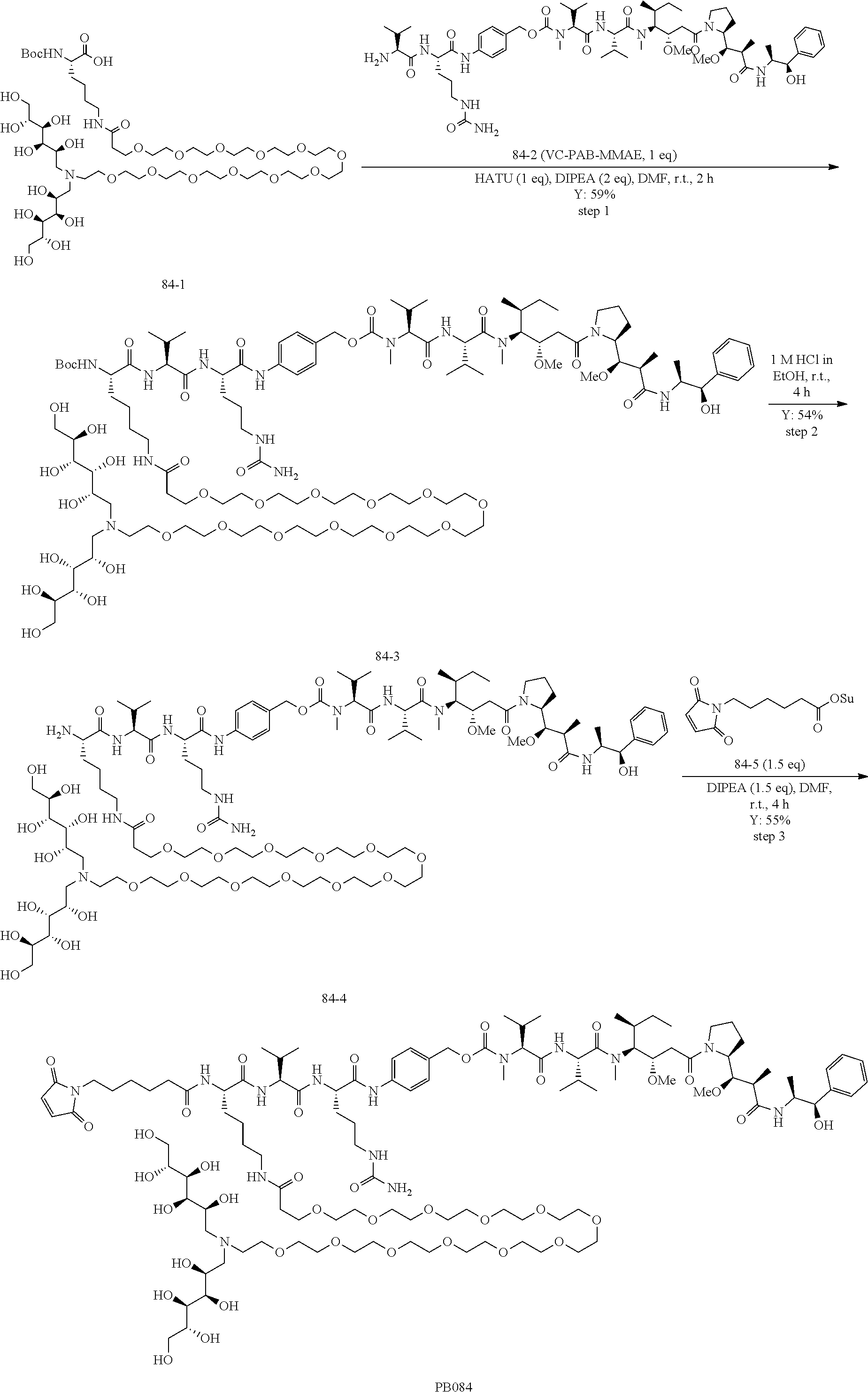

A Drug-Linker containing a PEG unit and a cleavable linker attached to MMAE (PB084) was prepared as follows:

### Step 1

A solution of compound 84-1 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-1, 500 mg, 0.445 mmol)), compound 84-2 ((2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (84-2, 522.71 mg, 0.445 mmol)) and DIPEA (172.31 mg, 1.336 mmol) in anhydrous DMF (1 mL) was stirred at room temperature, then a solution of HATU (169.29 mg, 0.445 mmol) in anhydrous DMF (1 mL) was added dropwise by syringe over 5 min. After addition, the resulting solution was stirred for another 2 h until LCMS indicated complete reaction. Then the completed reaction solution was purified directly by reverse phase flash chromatography (0.01% TFA) to yield compound 84-3 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-3, 600 mg, 0.263 mmol, 59.11%)) as a white solid. ESI m/z: 760.8 (M/3+H)⁺.

### Step 2

To the solution of compound 84-3 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-3, 580 mg, 0.254 mmol)) in ethanol (3 mL) was added 2M HCl in ethanol (3 mL, 0.254 mmol). Then the resulting pale yellow solution was stirred at room temperature for 4 h until LCMS showed that starting material was substantially consumed. The completed solution was cooled in ice water and neutralized with aqueous sodium bicarbonate solution. Solvent was removed under reduced pressure and the residue in the water layer was purified by reverse phase flash chromatography (0.01% TFA) to yield product 84-4 {4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-4, 300 mg, 0.138 mmol, 54.20%) as a white solid. ESI m/z: 727.3 (M/3+H)⁺, 718.3 (fragment piece, MMAE), 473.5 (linker fragment, ((2178-717-28-18)/3+H)⁺). ¹H NMR (400 MHz, DMSO-d6) δ 10.11 (s, 1H), 8.97-8.80 (m, 1H), 8.43 (d, J=8.8 Hz, 1H), 8.32 (d, J=7.2 Hz, 1H), 8.24-8.06 (m, 4H), 7.96-7.86 (m, 1.5H), 7.66 (d, J=8.8 Hz, 0.5H), 7.58 (d, J=7.6 Hz, 2H), 7.35-7.24 (m, 6H), 7.18-7.16 (m, 1H), 6.07 (s, 1H), 5.55-5.32 (m, 4H), 5.12-4.95 (m, 2H), 4.87-4.37 (m, 9H), 4.29-4.23 (m, 2H), 4.04-3.93 (m, 4H), 3.88-3.84 (m, 1H), 3.82-3.77 (m, 2H), 3.69-3.67 (m, 2H), 3.62-3.56 (m, 8H), 3.53-3.46 (m, 44H), 3.30-3.12 (m, 14H), 3.03-2.83 (m, 10H), 2.46-2.39 (m, 1H), 2.32-2.23 (m, 3H), 2.15-1.85 (m, 5H), 1.85-1.55 (m, 6H), 1.55-1.24 (m, 11H), 1.06-0.97 (m, 8H), 0.92-0.75 (m, 29H) ppm. Two protons of carboxyl group on TFA were revealed.

### Step 3

A solution of compound 84-4 {4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (84-4, 200 mg, 0.092 mmol) and compound 84-5 (2,5-dioxopyrrolidin-1-yl 6-(2,5-dioxo-2,5-dihydro-1H-pyrrol-1-yl)hexanoate (84-5, 42.39 mg, 0.138 mmol)) in anhydrous DMF (4 mL) was stirred at room temperature for 5 min, then a solution of DIPEA (23.67 mg, 0.184 mmol) in DMF (1 mL) was added dropwise by syringe over 5 min. After addition, the resulting solution was stirred for another 4 hr until LCMS indicated the starting amine was substantially consumed. Then the reaction solution was purified by Prep-HPLC (0.01% FA) to yield the desired fractions, which were freeze-dried to yield PB084 ({4-[(2S)-5-(carbamoylamino)-2-[(2S)-2-[(2S)-2-[6-(2,5-dioxo-2,5-dihydro-1 H-pyrrol-1-yl)hexanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB084, 120 mg, 0.051 mmol, 55.11%)) as a white solid. ESI m/z: 1187.2 (M/3+H)⁺, 791.7 (M/3+H)⁺, 718.5 (fragment piece, MMAE), 538.2 (linker fragment, (M-717-28-18)/3+H)⁺). Retention time 6.558 min (HPLC). ¹H NMR (400 MHz, DMSO-d6) δ 10.04 (s, 1H), 8.36-8.28 (m, 0.5 H), 8.13 (d, J=6.8 Hz, 1H), 8.13-8.06 (m, 0.5H), 7.96 (d, J=8.0 Hz, 1H), 7.91 (d, J=8.4 Hz, 0.5H), 7.84-7.80 (m, 1H), 7.68-7.65 (m, 1.5H), 7.59-7.56 (m, 2H), 7.34-7.24 (m, 6H), 7.20-7.16 (m, 1H), 6.99 (s, 2H), 5.99 (d, J=5.6 Hz, 1H), 5.44-5.43 (m, 3H), 5.37 (d, J=4.8 Hz, 0.5H), 5.12-4.95 (m, 2H), 4.78-4.40 (m, 12H), 4.35-4.17 (m, 3.5H), 4.04-3.91 (m, 3H), 3.79-3.55 (m, 12H), 3.54-3.46 (m, 48H), 3.35-3.11 (m, 14H), 3.06-2.83 (m, 10H), 2.43-2.39 (m, 1H), 2.31-2.22 (m, 3H), 2.14-1.16 (m, 30H), 1.05-0.97 (m, 6H), 0.89-0.75 (m, 27H) ppm.

### Example 16: Preparation of a Drug-Linker Containing a PEG Unit and a Cleavable Linker Attached to SN-38 (PB085)

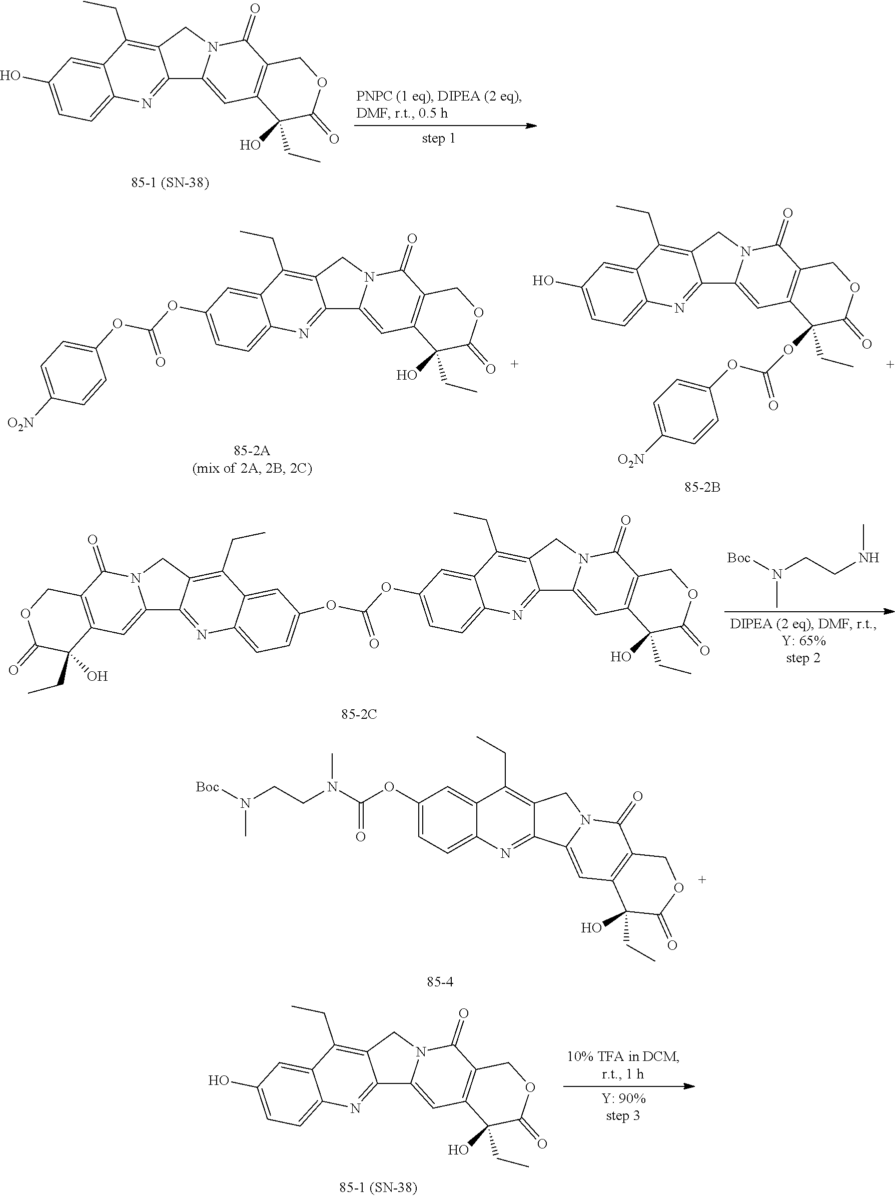

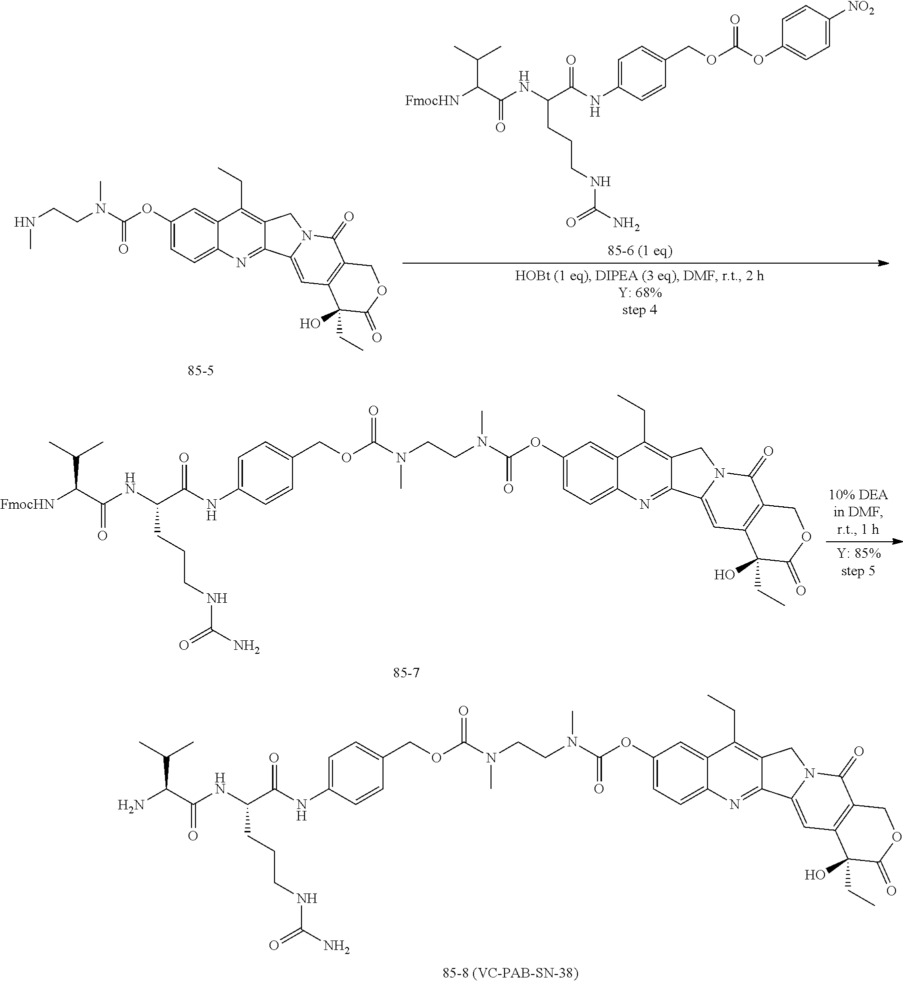

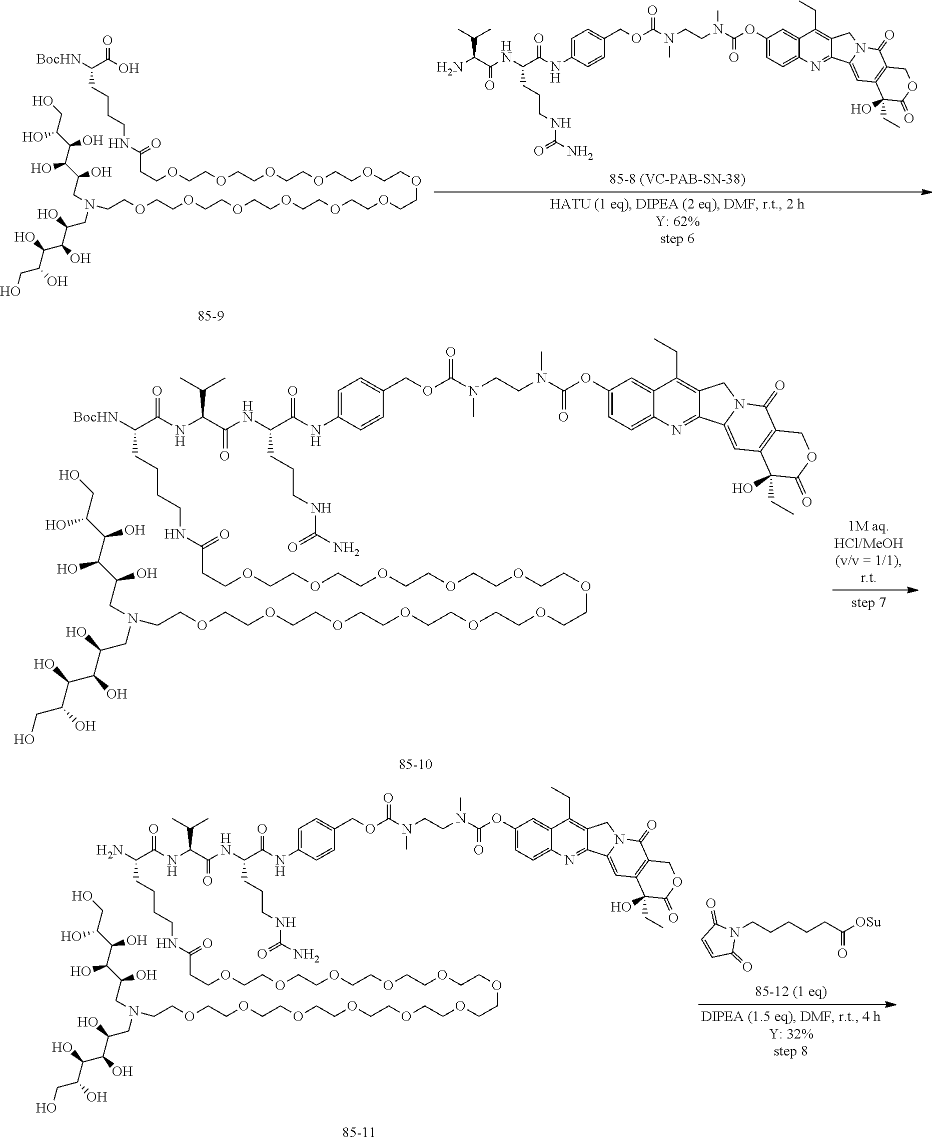

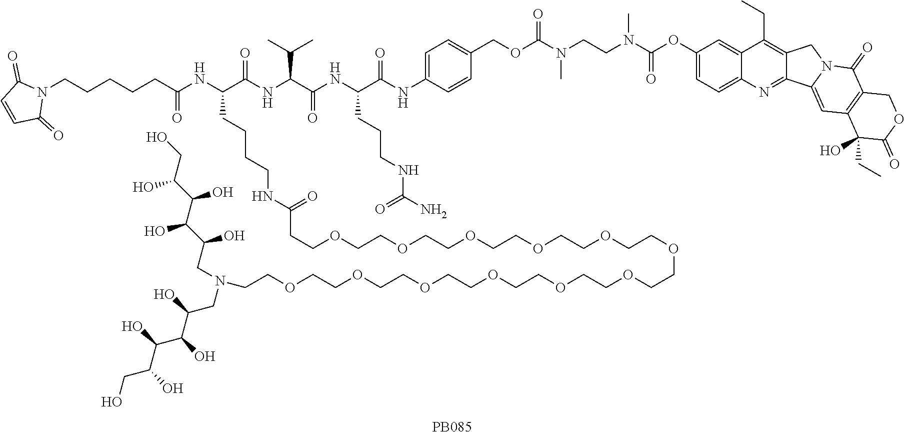

A Drug-Linker containing a PEG unit and a cleavable linker attached to SN-38 (PB085) was prepared as follows:

### Step 1

A pale yellow mixture of compound 85-1 ((19S)-10,19-diethyl-7,19-dihydroxy-17-oxa-3,13-diazapentacyclo[11.8.0.O\hat{ }{2,11}0.0{4,9}0.0{15,20}]henicosa-1(21),2,4,6,8,10,15(20)-heptaene-14,18-dione (SN-38) (85-1, 460 mg, 1.173 mmol)) and DIPEA (302.76 mg, 2.347 mmol) in anhydrous DMF (4 mL) was stirred at room temperature, and a solution of Bis(4-nitrophenyl) carbonate (PNPC, 356.73 mg, 1.173 mmol) in anhydrous DMF (2 mL) was added dropwise over 10 min. Upon the addition, the pale yellow mixture turned into light yellow mixture, and the materials dissolved slowly as the reaction proceeded. After addition, the resulting yellow clear solution was stirred at room temperature for another 30 min and then monitored by LCMS. The spectra indicated the complete consumption of starting material SN-38, the desired phenol-activated product was formed as major new peak, along with a side product alcohol-activated carbonate. The reaction solution was used directly for the next step. ESI m/z=558.2 (M+H)⁺.

### Step 2

The DMF solution of crude activated carbonate (mix of 85-2A, 85-2B, 85-2C) from last step was treated with compound 85-3 (tert-butyl N-methyl-N-[2-(methylamino)ethyl]carbamate (85-3, 264.63 mg, 1.408 mmol)) and DIPEA (302.63 mg, 2.346 mmol). After the reaction solution was stirred for 1h, LCMS indicated complete conversion. The desired product 85-4 and SN-38 were both detected. The reaction solution was purified by reverse phase flash chromatography (0.01% TFA) to give desired product 85-4 ((19S)-10,19-diethyl-19-hydroxy-14,18-dioxo-17-oxa-3,13-diazapentacyclo[11.8.0.0{2,11}0.0{4,9}0.0{15,20}]henicosa-1(21),2,4,6,8,10,15(20)-heptaen-7-yl N-(2-{[(tert-butoxy)carbonyl](methyl)amino}ethyl)-N-methylcarbamate (85-4, 467 mg, 0.771 mmol, 65.70%)) as a pale yellow solid. ESI m/z: 607.3 (M+H)⁺. ¹H NMR (400 MHz, DMSO-d₆) δ 10.10-10.01 (m, 1H), 8.18 (d, J=9.2 Hz, 1H), 8.10 (d, J=7.2 Hz, 1H), 7.93-7.86 (m, 3H), 7.73 (t, J=8.0 Hz, 2H), 7.62-7.52 (m, 3H), 7.43-7.26 (m, 8H), 6.52 (s, 1H), 5.97 (s, 1H), 5.45-5.34 (m, 5H), 5.09-5.00 (m, 2H), 4.44-4.40 (m, 1H), 4.32-4.22 (m, 3H), 3.93 (t, J=7.6 Hz, 1H), 3.65-3.50 (m, 7H), 3.19-3.12 (m, 2H), 3.04-2.89 (m, 7H), 2.02-1.914 (m, 1H), 1.91-1.74 (m, 2H), 1.74-1.52 (m, 2H), 1.52-1.36 (m, 2H), 1.36-1.26 (m, 2H), 0.91-0.84 (m, 9H) ppm.

### Step 3

The solution of compound 85-4 ((19S)-10,19-diethyl-19-hydroxy-14,18-dioxo-17-oxa-3,13-diazapentacyclo[11.8.0.0\hat{ }{2,11}0.0{4,9}0.0\hat{ }{15,20}]henicosa-1(21),2,4,6,8,10,15(20)-heptaen-7-yl N-(2-{[(tert-butoxy)carbonyl](methyl)amino}ethyl)-N-methylcarbamate (85-4, 600 mg, 0.990 mmol)) in DCM (1.8 mL) was stirred at room temperature, then TFA (0.2 mL, 2.693 mmol) was added. The resulting solution was stirred for 1h until LCMS showed that the reaction was completed. All solvent and TFA were evaporated off with a rotary evaporator, then the residue was purified by reverse phase flash chromatography (0.01% TFA) to yield the expected fractions, which were lyophilized to give a TFA salt of product 85-5 ((19S)-10,19-diethyl-19-hydroxy-14,18-dioxo-17-oxa-3,13-diazapentacyclo[11.8.0.0{2,11}0.0{4,9}0.0{15,20}]henicosa-1(21),2,4,6,8,10,15(20)-heptaen-7-yl N-methyl-N-[2-(methylamino)ethyl]carbamate (85-5, 550 mg, 0.889 mmol, 89.75%)) as a pale yellow solid. ESI m/z: 254.3 (M/2+H)⁺, 507.3 (M+H)⁺. ¹H NMR (400 MHz, DMSO-d₆) δ 9.25 (s, 1H), 9.16 (s, 1H), 8.19 (d, J=9.2 Hz, 1H), 8.13 (s, 1H), 7.84-7.80 (m, 1H), 7.35 (s, 1H), 5.45 (s, 2H), 5.35 (s, 2H), 3.84-3.82 (m, 1H), 3.67-3.64 (m, 1H), 3.25-3.17 (m, 6H), 3.01 (s, 1H), 2.64-2.57 (m, 3H), 1.92-1.84 (m, 2H), 1.31 (t, J=7.2 Hz, 3H), 0.89 (t, J=7.2 Hz, 3H) ppm. One proton of carboxyl group on TFA was revealed.

### Step 4

A yellow solution of HOBt (78.39 mg, 0.581 mmol) and compound 85-6 ({4-[(2S)-5-(carbamoylamino)-2-[(2S)-2-({[(9H-fluoren-9-yl)methoxy]carbonyl}amino)-3-methylbutanamido]pentanamido]phenyl}methyl 4-nitrophenyl carbonate (85-6, 444.77 mg, 0.581 mmol)) in anhydrous DMF (2 mL) was stirred at room temperature, then a solution of DIPEA (224.71 mg, 1.742 mmol) in anhydrous DMF (2 mL) was added dropwise by syringe. The color of the reaction solution turned to brown upon the addition of base. After the addition, the resulting brown solution was stirred for another 1 h until all starting amine was consumed (monitored by LCMS). The reaction solution was purified directly by reverse phase flash chromatography (0.01% TFA) to yield compound 85-7 ({4-[(2S)-5-(carbamoylamino)-2-[(2S)-2-({[(9H-fluoren-9-yl)methoxy]carbonyl}amino)-3-methylbutanamido]pentanamido]phenyl}methyl N-{2-[({[(19S)-10,19-diethyl-19-hydroxy-14,18-dioxo-17-oxa-3,13-diazapentacyclo[11.8.0.0{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-7, 450 mg, 0.397 mmol, 68.33%)) as a yellow solid. ESI m/z: 568.4 (M/2+H)⁺. ¹H NMR (400 MHz, DMSO-d₆) δ 10.10-10.01 (m, 1H), 8.18 (d, J=9.2 Hz, 1H), 8.10 (d, J=7.2 Hz, 1H), 7.93-7.86 (m, 3H), 7.73 (t, J=8.0 Hz, 2H), 7.62-7.52 (m, 3H), 7.43-7.26 (m, 8H), 6.52 (s, 1H), 5.97 (s, 1H), 5.45-5.34 (m, 5H), 5.09-5.00 (m, 2H), 4.44-4.40 (m, 1H), 4.32-4.22 (m, 3H), 3.93 (t, J=7.6 Hz, 1H), 3.65-3.50 (m, 7H), 3.19-3.12 (m, 2H), 3.04-2.89 (m, 7H), 2.02-1.914 (m, 1H), 1.91-1.74 (m, 2H), 1.74-1.52 (m, 2H), 1.52-1.36 (m, 2H), 1.36-1.26 (m, 2H), 0.91-0.84 (m, 9H) ppm.

### Step 5

To a yellow solution of compound 85-7 ({4-[(2S)-5-(carbamoylamino)-2-[(2S)-2-({[(9H-fluoren-9-yl)methoxy]carbonyl}amino)-3-methylbutanamido]pentanamido]phenyl}methyl N-{2-[({[(19S)-10,19-diethyl-19-hydroxy-14,18-dioxo-17-oxa-3,13-diazapentacyclo[11.8.0.0{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-7, 450 mg, 0.397 mmol)) in anhydrous DMF (1.8 mL) was added diethyl amine (0.2 mL, 1.941 mmol). The solution was stirred for another 1 h until all starting material was consumed (monitored by LCMS). Then the reaction solution was evaporated with a rotary evaporator to remove most of the diethyl amine, and the residue was purified by reverse phase flash chromatography (0.01% TFA) to yield compound 85-8, VC-PAB-SN-38 TFA salt ({4-[(2S)-2-[(2S)-2-amino-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-8, VC-PAB-SN-38 TFA salt, 310 mg, 0.340 mmol, 85.62%)), as a yellow solid. ESI m/z: 456.9 (M/2+H)⁺, 912.4 (M+H)⁺. ¹H NMR (400 MHz, DMSO-d₆) δ 10.18 (s, 0.6H), 10.14 (s, 0.4H), 8.67 (d, J=8.0 Hz, 1H), 8.18 (d, J=7.6 Hz, 1H), 8.08 (brs, 3H), 7.94 (d J=7.6 Hz, 1H), 7.58-7.50 (m, 3H), 7.34-7.27 (m, 3H), 6.54 (s, 1H), 6.04 (t, J=5.6 Hz, 1H), 5.53-5.34 (m, 6H), 5.06-5.00 (m, 2H), 4.55-4.45 (m, 1H), 3.70-3.60 (m, 2H), 3.60-3.50 (m, 1H), 3.50-3.46 (m, 2H), 3.21-3.12 (m, 3H), 3.05-2.89 (m, 8H), 1.93-1.82 (m, 2H), 1.77-1.55 (m, 2H), 1.55-1.38 (m, 2H), 1.38-1.22 (m, 3H), 0.95-0.87 (m, 9H) ppm. One proton of carboxyl group on TFA overlapped with amino group at 8.08 ppm.

### Step 6

A solution of compound 85-8 ({4-[(2S)-2-[(2S)-2-amino-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-8, VC-PAB-SN-38, 240 mg, 0.263 mmol)), compound 85-9 ((2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (85-9, 308.95 mg, 0.263 mmol)) and DIPEA (67.89 mg, 0.526 mmol) in anhydrous DMF (3 mL) was stirred at room temperature for 5 min, then a solution of HATU (100.06 mg, 0.263 mmol) in anhydrous DMF (3 mL) was added dropwise by syringe. The resulting yellow solution was stirred for another 2 h until LCMS indicated complete reaction. The reaction solution was purified directly by reverse phase liquid chromatography (0.01% TFA) to yield compound 85-10 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-10, 340 mg, 0.164 mmol, 62.48%)) as a pale yellow solid. ESI m/z: 690.3 (M/3+H)⁺. ¹H NMR (400 MHz, DMSO-d₆) δ 10.03 (s, 1H), 8.20-8.10 (m, 3H), 7.94-7.90 (m, 1H), 7.80 (t, J=13.2 Hz, 1H), 7.59-7.53 (m, 4H), 7.34-7.27 (m, 3H), 7.01 (d, J=8.0 Hz, 1H), 6.53 (s, 1H), 5.99 (t, J=5.2 Hz, 1H), 5.45-5.35 (m, 7H), 5.05-5.00 (m, 2H), 4.84-4.37 (m, 8H), 4.25 (t, J=7.6 Hz, 1H), 4.04-3.96 (m, 2H), 3.92-3.85 (m, 1H), 3.80-3.77 (m, 2H), 3.70-3.67 (m, 3H), 3.63-3.53 (m, 18H), 3.50-3.40 (m, 36H), 3.36-3.27 (m, 11H), 3.09-3.12 (m, 3H), 3.04-2.89 (m, 9H), 2.29 (t, J=6.0 Hz, 2H), 1.99-1.83 (m, 3H), 1.69-1.53 (m, 3H), 1.53-1.26 (m, 19H), 0.91-0.81 (m, 9H) ppm. One proton of carboxyl group on TFA appeared between 8.20-8.10 ppm.

### Step 7

To a solution of compound 85-10 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-10, 100 mg, 0.048 mmol)) in methanol (3 mL) was added 2M HCl in methanol (1 mL) slowly with stirring under room temperature. Upon addition, the clear solution became bright yellow. The solution was kept stirring for 2 h until LCMS indicated full deprotection. Then the solution was cooled to −20° C. and neutralized with DIPEA; as a result, the yellow reaction solution turned into colorless solution. Organic solvent was then evaporated off with rotary evaporator below 30° C. to yield a pale yellow residue, which was dissolved in water and purified by reverse phase flash chromatography (0.01% TFA) to yield the desired fractions (pH 3-4, LCMS showed the purity of fractions around 60%-58%). After lyophilization, a yellow solid (˜100 mg) was obtained with a purity of 63%-64%. Then the solid was purified again by reverse phase flash chromatography (neutral eluent) to yield the desired fractions (pH 6-7) with a purity of around 48%-51%. The collected fractions were lyophilized to yield compound 85-11 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-11, 80 mg, 0.041 mmol, 84.07%)) as pale yellow solid. The product was used directly in next step as impure material. ESI m/z: 657.0 (M/3+H)⁺, 985.2 (M/2+H)⁺.

### Step 8

To a 50 mL round-bottomed flask was added compound 85-11 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-{2-[({[(19S)-10,19-diethyl-19-hydroxy-14,18-dioxo-17-oxa-3,13-diazapentacyclo[11.8.0.0\hat{ }{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (85-11, 80 mg crude with purity 45%, 0.041 mmol)), compound 85-12 (2,5-dioxopyrrolidin-1-yl 6-(2,5-dioxo-2,5-dihydro-1 H-pyrrol-1-yl)hexanoate (85-12, 12.52 mg, 0.041 mmol)) and anhydrous DMF (2 mL). The solution was stirred at room temperature and a solution of DIPEA (7.87 mg, 0.061 mmol) in anhydrous DMF (2 mL) was added dropwise by syringe over 5 min. After addition, the solution was stirred for another 4h until LCMS indicated all starting amine was consumed. Then the completed reaction solution was neutralized with TFA and purified directly by Prep-HPLC (0.01% TFA) to yield the desired fractions, which were freeze-dried to yield a TFA salt of compound PB085 ({4-[(2S)-5-(carbamoylamino)-2-[(2S)-2-[(2S)-2-[6-(2,5-dioxo-2,5-dihydro-1 H-pyrrol-1-yl)hexanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-{2-[({[(19S)-10,19-diethyl-19-hydroxy-14,18-dioxo-17-oxa-3,13-diazapentacyclo[11.8.0.0{2,11}0.0{4,9}0.0{15,20}]henicosa-1(21),2,4,6,8,10,15(20)-heptaen-7-yl]oxy}carbonyl)(methyl)amino]ethyl}-N-methylcarbamate (PB085, 13 mg, 0.006 mmol, yield 32% calculated on staring amine content)) as a white solid. ESI m/z: 1081.3 (M/2+H)⁺, 721.3 (M/3+H)⁺, 541.5 (M/4+H)⁺, retention time 5.819 min (HPLC). ¹H NMR (400 MHz, DMSO-d6) δ 10.00 (s, 0.6 H), 9.98 (s, 0.4 H), 8.20-8.08 (m, 3H), 7.94 (d, J=8.0 Hz, 2H), 7.79 (t, J=5.6 Hz, 1H), 7.66-7.52 (m, 4H), 7.34-7.26 (m, 3H), 6.99 (s, 2H), 6.53 (s, 1H), 5.98 (t, J=5.6 Hz, 1H), 5.45-5.41 (m, 5H), 5.35 (s, 2H), 5.05-4.99 (m, 2H), 4.82-4.73 (m, 2H), 4.59-4.49 (m, 4H), 4.49-4.32 (m, 3H), 4.27-4.15 (m, 2H), 4.04-3.95 (m, 2H), 3.82-3.75 (m, 2H), 3.69-3.44 (m, 65H), 3.19-3.12 (m, 3H), 3.03-2.89 (m, 11H), 2.29 (t, J=6.4 Hz, 2H), 2.14-2.08 (m, 2H), 2.02-1.84 (m, 3H), 1.72-1.60 (m, 3H), 1.55-1.15 (m, 18H), 0.90-0.80 (m, 9H) ppm. One proton of carboxyl group on TFA was revealed between 8.20-8.08 ppm.

### Example 17: Preparation of a Drug-Linker Containing a PEG Unit and a Cleavable Linker Attached to Exatecan (PB086)

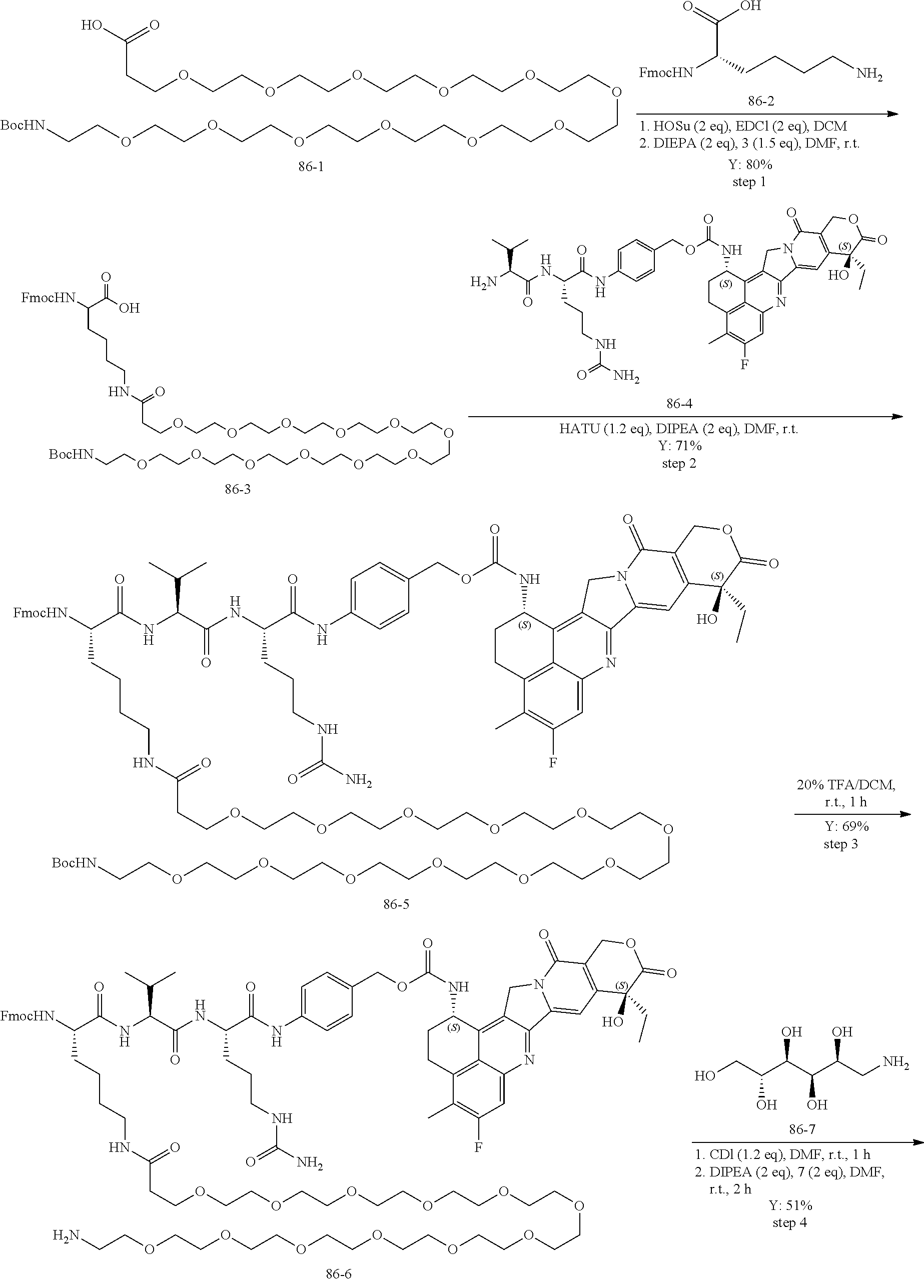

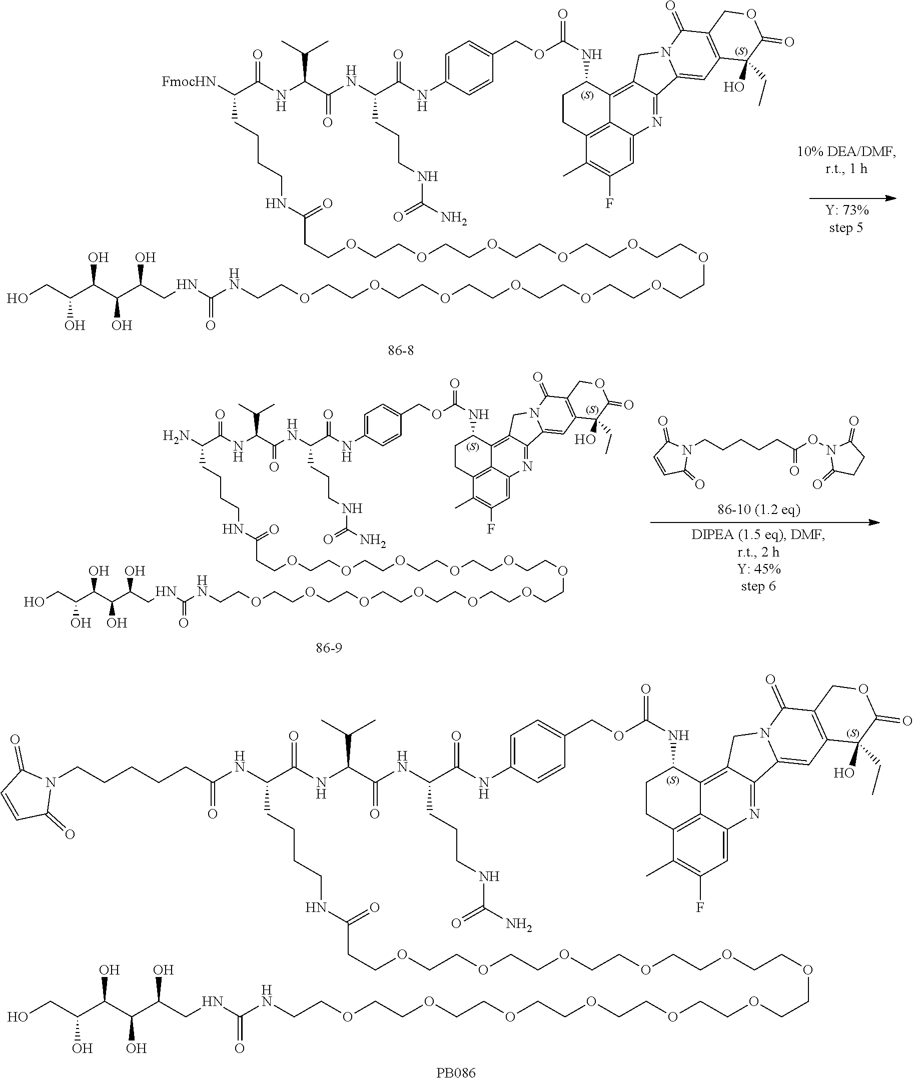

A Drug-Linker containing a PEG unit and a cleavable linker attached to exatecan (PB086) was prepared as follows:

### Step 1

A solution of compound 86-1 (1-{[(tert-butoxy)carbonyl]amino}-3,6,9,12,15,18,21,24,27,30,33,36-dodecaoxanonatriacontan-39-oic acid (86-1, 500 mg, 0.697 mmol)) and HOSu (160.39 mg, 1.395 mmol) in anhydrous DCM (14 mL) was stirred at room temperature for 5 min, then EDCl (267.36 mg, 1.395 mmol) was added. The resulting solution was stirred for another 1h, then diluted with more DCM (20 mL) and washed with water (20 mL). The organic layer was collected, and the water layer was extracted with more DCM (20 mL). The combined DCM layer was dried over sodium sulfate, then filter and concentrated to yield crude 2,5-dioxopyrrolidin-1-yl 2,2-dimethyl-4-oxo-3,8,11,14,17,20,23,26,29,32,35,38,41-tridecaoxa-5-azatetratetracontan-44-oate (800 mg, quantative yield) as colorless oil. ESI m/z: 715.5 (M-100+H)⁺; 837.5 (M+Na)⁺. The activated ester (800 mg crude, 0.697 mmol) was dissolved in anhydrous DMF (4 mL), then compound 86-2 ((2S)-6-amino-2-({[(9H-fluoren-9-yl)methoxy]carbonyl}amino)hexanoic acid, TFA salt (86-2, 503.93 mg, 1.045 mmol)) and DIPEA (179.83 mg, 1.394 mmol) were added. The reaction solution was stirred at room temperature for 1h until all activated ester was consumed (monitored by LCMS). Then the resulting solution was purified directly by reverse phase flash chromatography (0.01% TFA) to yield compound 86-3 ((2S)-6-(1-{[(tert-butoxy)carbonyl]amino}-3,6,9,12,15,18,21,24,27,30,33,36-dodecaoxanonatriacontan-39-amido)-2-({[(9H-fluoren-9-yl)methoxy]carbonyl}amino)hexanoic acid (86-3, 600 mg, 0.562 mmol, 80.58%)) as pale yellow oil. ESI m/z: 484.9 ((M-100)/2+H)⁺, 968.7(M-100+H)⁺. ¹H NMR (400 MHz, DMSO-d6) δ 12.64 (s, 1H), 7.90 (d, J=7.2 Hz, 2H), 7.82 (t, J=5.6 Hz, 1H), 7.73 (d, J=7.2 Hz, 2H), 7.62 (d, J=8.0 Hz, 1H), 7.45-7.40 (m, 2H), 7.36-7.31 (m, 2H), 7.76 (t, J=5.6 Hz, 1H), 4.29-4.21 (m, 3H), 3.94-3.88 (m, 1H), 3.68-3.56 (m, 4H), 3.51-3.47 (m, 44H), 3.07-3.00 (m, 4H), 2.29 (t, J=6.4 Hz, 2H), 1.73-1.60 (m, 2H), 1.52-1.29 (m, 13H) ppm.

### Step 2

A solution of compound 86-3 ((2S)-6-(1-{[(tert-butoxy)carbonyl]amino}-3,6,9,12,15,18,21,24,27,30,33,36-dodecaoxanonatriacontan-39-amido)-2-({[(9H-fluoren-9-yl)methoxy]carbonyl}amino)hexanoic acid (86-3, 600 mg, 0.562 mmol)), HATU (256.43 mg, 0.674 mmol) and DIPEA (145.00 mg, 1.124 mmol) in anhydrous DMF (4 mL) was stirred at room temperature for 5 min, then compound 86-4 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (86-4, 471.91 mg, 0.562 mmol)) was added. The resulting solution was stirred for another 1 h until LCMS indicated complete reaction. Then the solution was purified directly by reverse phase flash chromatography (0.01% TFA) to yield compound 86-5 (tert-butyl N-(38-{[(5S)-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-({[(9H-fluoren-9-yl)methoxy]carbonyl}amino)pentyl]carbamoyl}-3,6,9,12,15,18,21,24,27,30,33,36-dodecaoxaoctatriacontan-1-yl)carbamate (86-5, 750 mg, 0.397 mmol, 70.60%)) as an off-white solid. ESI m/z: 597.9 ((M-100)/3+H)⁺, 896.1 ((M-100)/2+H)⁺, 946.7 (M/2+H)⁺.

### Step 3

To the solution of compound 86-5 (tert-butyl N-(38-{[(5S)-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-({[(9H-fluoren-9-yl)methoxy]carbonyl}amino)pentyl]carbamoyl}-3,6,9,12,15,18,21,24,27,30,33,36-dodecaoxaoctatriacontan-1-yl)carbamate (86-5, 700 mg, 0.370 mmol)) in DCM (8 mL) TFA (2 mL, 26.925 mmol) was added slowly. The resulting yellow solution was stirred at room temperature for 1 h to complete. TFA and solvent were evaporated with a rotary evaporator, and the residue was purified by reverse phase flash chromatography (0.01% TFA) to yield the expected fractions, which were lyophilized to yield product 86-6 ((9H-fluoren-9-yl)methyl N-[(1S)-5-(1-amino-3,6,9,12,15,18,21,24,27,30,33,36-dodecaoxanonatriacontan-39-amido)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (86-6, 460 mg, 0.257 mmol, 69.38%)) as a white solid. ESI m/z: 896.6 (M/2+H)⁺, 597.9(M/3+H)⁺.

### Step 4

To a solution of CDI (0.029 mL, 0.234 mmol) in anhydrous DMF (4 mL) was added a solution of compound 86-6 ((9H-fluoren-9-yl)methyl N-[(1S)-5-(1-amino-3,6,9,12,15,18,21,24,27,30,33,36-dodecaoxanonatriacontan-39-amido)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (86-6, 350 mg, 0.195 mmol)) in anhydrous DMF (4 mL) dropwise by syringe over 5 min. After addition, the pale yellow solution was stirred at room temperature for another 1 h to completion (monitored by LCMS). Activated intermediate (9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[(1H-imidazole-1-carbonyl)amino]-3,6,9,12,15,18,21,24,27,30,33,36-dodecaoxanonatriacontan-39-amido}pentyl]carbamate was detected as major peak. ESI m/z: 629.3 (M/3+H)⁺, 943.3 (M/2+H)⁺.

To the reaction solution of activated intermediate (0.195 mmol) in DMF (4 mL) above was added compound 86-7 ((2R,3R,4R,5S)-6-aminohexane-1,2,3,4,5-pentol (86-7, 70.59 mg, 0.390 mmol)) and DIPEA (50.31 mg, 0.390 mmol). The resulting solution was stirred at room temperature for 2 h until all materials were consumed. Then the solution was purified directly by reverse phase flash chromatography (0.01% TFA) to yield compound 86-8 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]pentyl]carbamate (86-8, 200 mg, 0.100 mmol, 51.33%)) as a pale yellow solid. ESI m/z: 999.8(M/2+H)⁺, 666.9 (M/3+H)⁺.

### Step 5

A solution of compound 86-8 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]pentyl]carbamate (86-8, 200 mg, 0.100 mmol)) in DMF (3.6 mL) was stirred at room temperature and diethyl amine (0.4 mL, 3.883 mmol) was added. The resulting solution was stirred for 30 min. Then diethyl amine was evaporated under vacuo, the residue in DMF was neutralized with formic acid and purified by reverse phase flash chromatography (0.01% TFA) to yield the expected product 86-9 ({4-[(2S)-2-[(2S)-2-[(2S)-2-amino-6-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (86-9, 130 mg, 0.073 mmol, 73.14%)) as a pale yellow solid. ESI m/z: 592.9 (M/3+H)⁺, 889.0 (M/2+H)⁺. ¹H NMR (400 MHz, DMSO-d6) δ 10.09 (s, 1H), 8.42 (d, J=8.4 Hz, 1H), 8.31 (d, J=6.8 Hz, 1H), 8.12-8.04 (m, 4H), 7.85 (t, J=5.6 Hz, 1H), 7.78 (d, J=10.8 Hz, 1H), 7.60 (d, J=7.6 Hz, 2H), 7.37 (d, J=8.8 Hz, 2H), 7.32 (s, 1H), 6.54 (brs, 1H), 6.12 (t, J=5.6 Hz, 1H), 6.04 (t, J=5.6 Hz, 1H), 5.97 (t, J=5.6 Hz, 1H), 5.54-5.45 (m, 3H), 5.40-5.23 (m, 3H), 5.09 (s, 2H), 4.44-4.38 (m, 2H), 4.30-4.27 (m, 1H), 3.89-3.81 (m, 1H), 3.69-3.54 (m, 6H), 3.54-3.45 (m, 48H), 3.26-3.13 (m, 6H), 3.13-3.05 (m, 4H), 3.05-2.98 (m, 3H), 2.98-2.89 (m, 2H), 2.37 (s, 3H), 2.29 (t, J=6.4 Hz, 2H), 2.24-2.10 (m, 2H), 2.04-1.93 (m, 1H), 1.93-1.82 (m, 2H), 1.72-1.54 (m, 4H), 1.45-1.23 (m, 6H), 0.92-0.86 (m, 9H) ppm. One proton of carboxyl group on TFA appeared between 8.12-8.04 ppm.

### Step 6

A solution of compound 86-9 ({4-[(2S)-2-[(2S)-2-[(2S)-2-amino-6-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (86-9, 60 mg, 0.034 mmol)) and compound 86-10 (2,5-dioxopyrrolidin-1-yl 6-(2,5-dioxo-2,5-dihydro-1H-pyrrol-1-yl)hexanoate (86-10, 12.50 mg, 0.041 mmol)) in anhydrous DMF (1.5 mL) was stirred at room temperature, then DIPEA (6.54 mg, 0.051 mmol) was added. The resulting solution was stirred for another 2 h until LCMS indicated all starting amine was consumed. The resulting solution was neutralized with formic acid, then purified by Prep-HPLC (0.01% TFA) to yield the desired fractions, which were freeze-dried to yield PB086 ({4-[(2S)-5-(carbamoylamino)-2-[(2S)-2-[(2S)-2-[6-(2,5-dioxo-2,5-dihydro-1 H-pyrrol-1-yl)hexanamido]-6-[1-({[(2S,3R,4R,5R)-2,3,4,5,6-pentahydroxyhexyl]carbamoyl}amino)-3,6,9,12,15,18,21,24,27,30,33,36-dodecaoxanonatriacontan-39-amido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB086, 30 mg, 0.015 mmol, 45.09%)) as a white solid. ESI m/z: 985.1 (M/2+H)⁺, 657.3 (M/3+H)⁺, 493.3 (M/4+H)⁺, retention time 6.424 min (HPLC). ¹H NMR (400 MHz, DMSO-d6) δ 10.03 (s, 1H), 8.11 (d, J=7.2 Hz, 1H), 8.06 (d, J=8.8 Hz, 1H), 7.96 (d, J=8.0 Hz, 1H), 7.82-7.76 (m, 2H), 7.66 (d, J=8.8 Hz, 1H), 7.60 (d, J=8.4 Hz, 2H), 7.36 (d, J=8.4 Hz, 2H), 7.32 (s, 1H), 6.99 (s, 2H), 6.50 (brs, 1H), 6.11 (t, J=5.6 Hz, 1H), 6.00-5.94 (m, 2H), 5.45-5.38 (m, 3H), 5.35-5.24 (m, 3H), 5.08 (s, 2H), 4.40-4.17 (m, 4H), 3.59-3.45 (m, 54H), 3.24-3.16 (m, 4H), 3.16-3.09 (m, 4H), 3.04-2.90 (m, 7H), 2.38 (s, 3H), 2.29 (t, J=6.4 Hz, 2H), 2.23-2.13 (m, 2H), 2.13-2.06 (m, 2H), 2.01-1.96 (m, 1H), 1.96-1.81 (m, 2H), 1.73-1.54 (m, 4H), 1.48-1.29 (m, 10H), 1.24-1.14 (m, 4H), 0.90-0.81 (m, 9H) ppm.

### Example 18: Preparation of a Drug-Linker Containing a PEG Unit and a Cleavable Linker Attached to Exatecan (PB087)

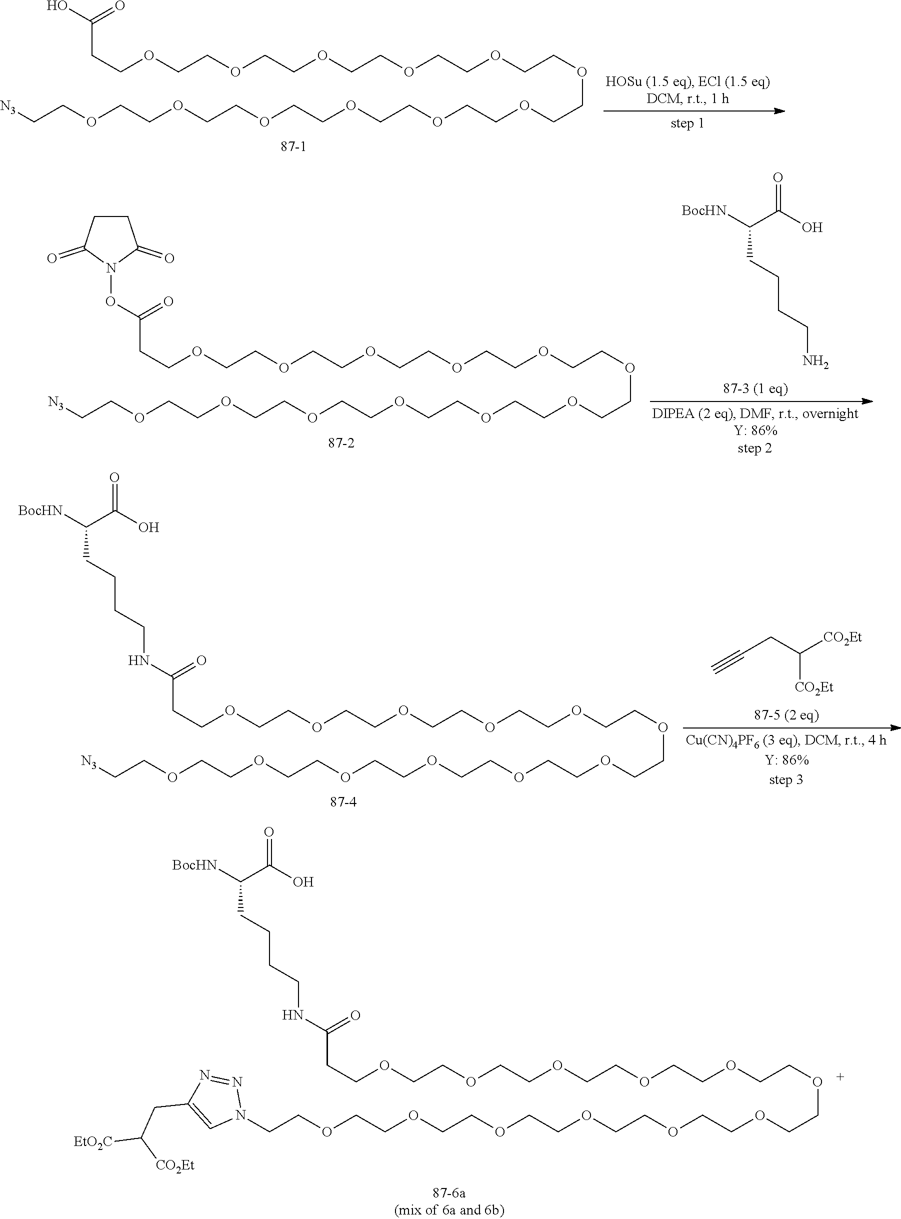 [SMILES: [N-]=[N+]=NCCOCCOCCOCCOCCOCCOCCOCCOCCOCCOCCOCCOCCC(=O)O ; [N-]=[N+]=NCCOCCOCCOCCOCCOCCOCCOCCOCCOCCOCCOCCOCCC(=O)ON1C(=O)CCC1=O ; CC(C)(C)OC(=O)N[C@@H](CCCCN)C(=O)O ; CC(C)(C)OC(=O)N[C@@H](CCCCNC(=O)CCOCCOCCOCCOCCOCCOCCOCCOCCOCCOCCOCCOCCN=[N+]=[N-])C(=O)O ; C#CCC(C(=O)OCC)C(=O)OCC ; CCOC(=O)C(Cc1cn(CCOCCOCCOCCOCCOCCOCCOCCOCCOCCOCCOCCOCCC(=O)NCCCC[C@H](NC(=O)OC(C)(C)C)C(=O)O)nn1)C(=O)OCC]

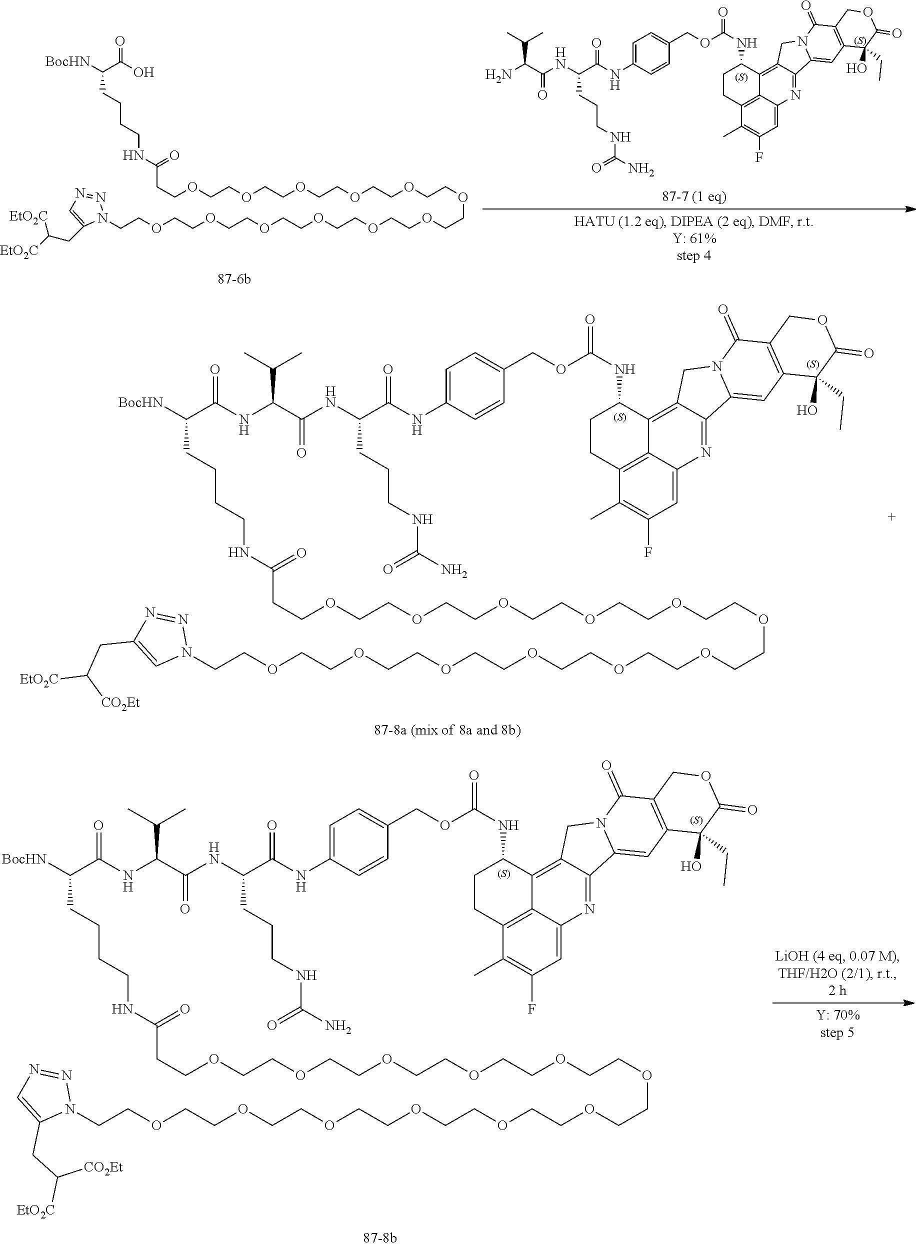

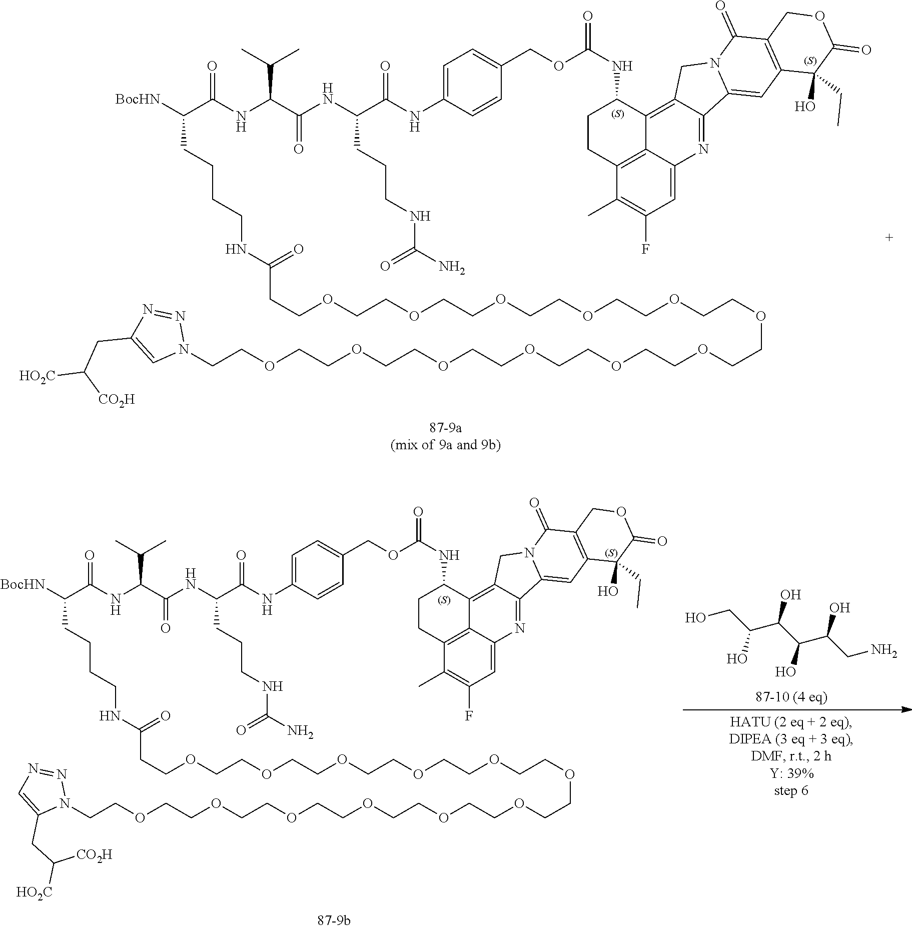

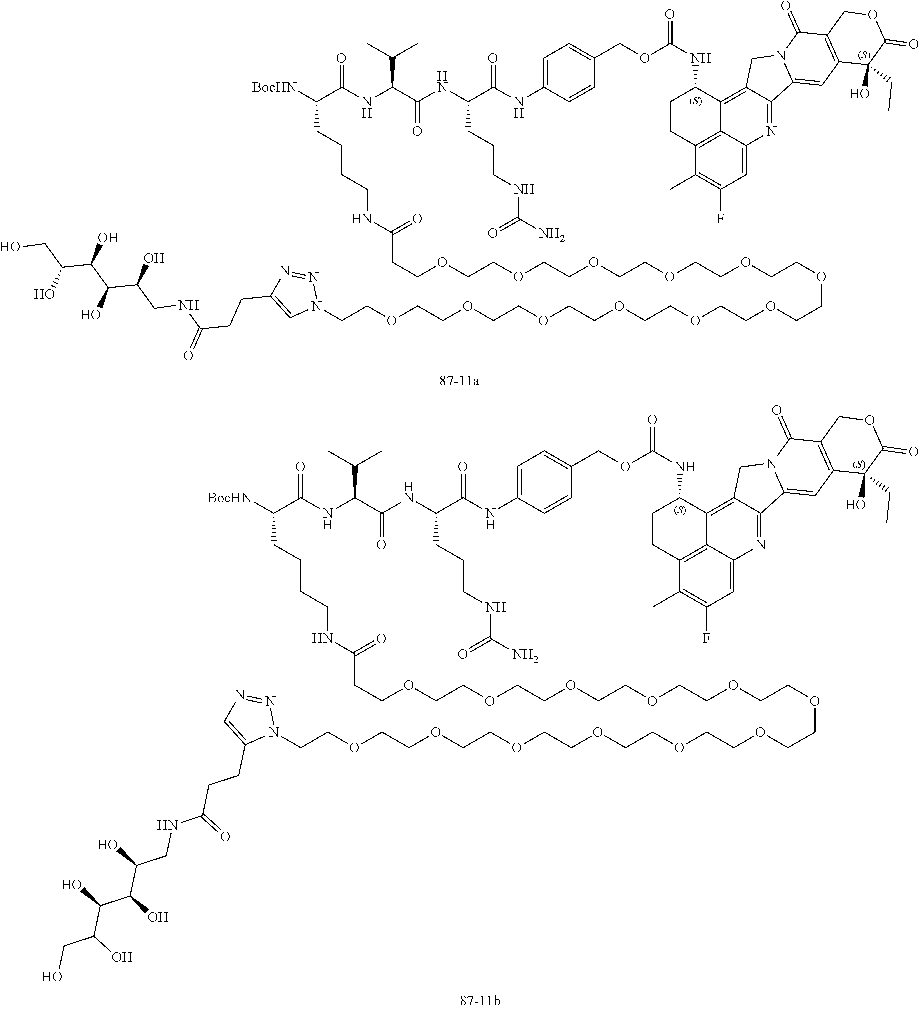

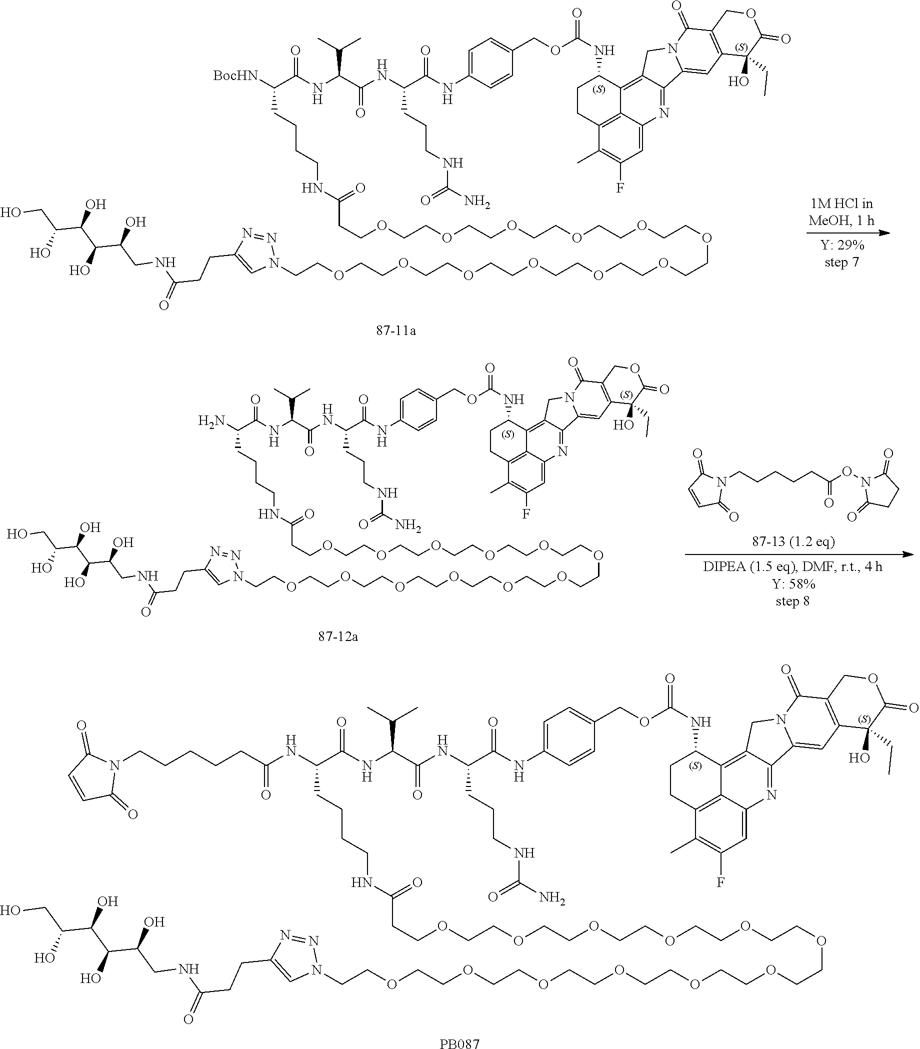

A Drug-Linker containing a PEG unit and a cleavable linker attached to exatecan (PB087) was prepared as follows:

### Steps 1 & 2

A solution of compound 87-1 (1-azido-3,6,9,12,15,18,21,24,27,30,33,36-dodecaoxanonatriacontan-39-oic acid (87-1, 300 mg, 0.466 mmol)) and HOSu (80.39 mg, 0.699 mmol) in anhydrous DCM (10 mL) was stirred at room temperature for 5 min, then EDCl (134.01 mg, 0.699 mmol) was added at room temperature. The resulting solution was stirred for another 1h, then diluted with more DCM (20 mL) and washed with water (20 mL), the organic layer was separated and the water layer was extracted with more DCM (20 mL*2). The combined DCM layer was dried over sodium sulfate, filtered and concentrated to yield crude activated ester (345 mg, quantative yield) as a colorless oil. ESI m/z: 741.5 (M+H)⁺, 763.4 (M+Na)⁺. The ester was dissolved in anhydrous DMF (4 mL), then compound 87-3 ((2S)-6-amino-2-{[(tert-butoxy)carbonyl]amino}hexanoic acid (87-3, 114.78 mg, 0.466 mmol)) and DIPEA (120.23 mg, 0.932 mmol) were added. The resulting mixture was stirred at room temperature for overnight until all starting amine was consumed, and the mixture turned into clear pale yellow solution. Then the completed reaction solution was purified directly by reverse phase flash chromatography (0.01% TFA) to yield compound 87-4((S)-1-azido-45-((tert-butoxycarbonyl)amino)-39-oxo-3,6,9,12,15,18,21,24,27,30,33,36-dodecaoxa-40-azahexatetracontan-46-oic acid (87-4, 350 mg, 0.401 mmol, 86.13%)) as a colorless oil. ESI m/z: 386.8 ((M-100)/2+H)⁺, 872.6 (M+H)⁺.

### Step 3

A solution of compound 87-4 ((S)-1-azido-45-((tert-butoxycarbonyl)amino)-39-oxo-3,6,9,12,15,18,21,24,27,30,33,36-dodecaoxa-40-azahexatetracontan-46-oic acid (87-4, 350 mg, 0.401 mmol)) and compound 87-5 (1,3-diethyl 2-(prop-2-yn-1-yl)propanedioate (87-5, 158.94 mg, 0.803 mmol)) in DCM (8 mL) was stirred at room temperature, then Cu(CN)₄PF₆ (447.94 mg, 1.204 mmol) was added. The resulting solution was stirred for another 4h until the reaction was completed (monitored by LCMS). The reaction solution was then concentrated and dissolved in acetonitrile, and filtered to remove the copper catalyst. The filtrate was purified by reverse phase flash chromatography (0.01% TFA) to yield a mixture (compounds 87-6a/87-6b, 350 mg, 0.327 mmol, 81.57%) of 1,4-disubstitued triazole isomer (2S)-2-{[(tert-butoxy)carbonyl]amino}-6-(1-{4-[3-ethoxy-2-(ethoxycarbonyl)-3-oxopropyl]-1H-1,2,3-triazol-1-yl}-3,6,9,12,15,18,21,24,27,30,33,36-dodecaoxanonatriacontan-39-amido)hexanoic acid and 1,5-disubstitued triazole isomer (2S)-2-{[(tert-butoxy)carbonyl]amino}-6-(1-{5-[3-ethoxy-2-(ethoxycarbonyl)-3-oxopropyl]-1H-1,2,3-triazol-1-yl}-3,6,9,12,15,18,21,24,27,30,33,36-dodecaoxanonatriacontan-39-amido)hexanoic acid as a white solid. ESI m/z: 485.9 ((M-100)/2+H)⁺, both regioselective isomers overlapped in LCMS.

### Step 4

A mixture of compounds 87-6a/87-6b (350 mg, 0.327 mmol of (2S)-2-{[(tert-butoxy)carbonyl]amino}-6-(1-{5-[3-ethoxy-2-(ethoxycarbonyl)-3-oxobutyl]-1H-1,2,3-triazol-1-yl}-3,6,9,12,15,18,21,24,27,30,33,36-dodecaoxanonatriacontan-39-amido)hexanoic acid and (2S)-2-{[(tert-butoxy)carbonyl]amino}-6-(1-{4-[3-ethoxy-2-(ethoxycarbonyl)-3-oxopropyl]-1H-1,2,3-triazol-1-yl}-3,6,9,12,15,18,21,24,27,30,33,36-dodecaoxanonatriacontan-39-amido)hexanoic acid), HATU (149.25 mg, 0.393 mmol) and DIPEA (23.99 mg, 0.186 mmol) in anhydrous DMF (2 mL) was stirred at room temperature for 5 min, then compound 87-7 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamate (87-7, 275.06 mg, 0.327 mmol)) was added. The resulting solution was stirred for another1 h until LCMS indicated complete reaction. The reaction solution was purified directly by reverse phase flash chromatography (0.01% TFA) to yield a mixture of compounds 87-8a/87-8b (380 mg, 0.201 mmol, 61.36% of 1,3-diethyl 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioate and its regioselective isomeri,3-diethyl 2-{[1-(38-{[(5S)-5-{[(tert-butoxy)carbonyl]amino}-5-{[(1S)-1-{[(1 S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-4-yl]methyl}propanedioate) as a white solid. ESI m/z: 449.4((M-100)/4+H)⁺, 632.1 (M/3+H)⁺, 947.7(M/2+H)⁺, retention time 8.205 min (HPLC). Both isomers overlapped as one peak in LCMS. ¹H NMR (400 MHz, DMSO-d6): δ 10.06 (s, 1H), 8.20 (d, J=7.2 Hz, 1H), 8.07 (d, J=8.0 Hz, 1H), 7.84-7.76 (m, 3H), 7.61 (d, J=8.4 Hz, 2H), 7.55 (d, J=8.4 Hz, 1H), 7.36 (d, J=8.8 Hz, 2H), 7.31 (s, 1H), 7.02 (d, J=8.0 Hz, 1H), 6.63-6.43 (m, 1H), 5.99 (s, 1H), 5.50-5.40 (m, 3H), 5.34-5.23 (m, 3H), 5.08 (s, 2H), 4.48-4.45 (m, 2H), 4.42-4.36 (m, 1H), 4.27-4.23 (m, 1H), 4.13-4.08 (m, 4H), 3.93-3.86 (m, 1H), 3.82 (t, J=7.6 Hz, 1H), 3.77 (t, J=4.2 Hz, 2H), 3.58-3.55 (m, 2H), 3.50-3.45 (m, 44H), 3.32-3.21 (m, 2H), 3.13 (d, J=8.0 Hz, 2H), 3.04-2.92 (m, 5H), 2.38 (s, 3H), 2.29 (d, J=6.4 Hz, 2H), 2.23-2.10 (m, 2H), 1.99-1.71 (m, 3H), 1.71-1.48 (m, 3H), 1.48-1.21 (m, 16H), 1.15 (t, J=6.8 Hz, 6H), 0.90-0.81 (m, 9H) ppm.

### Step 5

A mixture of compounds 87-8a/87-8b (1,3-diethyl 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-4-yl]methyl}propanedioate (87-8a/87-8b, 380 mg, 0.201 mmol) and 1,3-diethyl 2-{[1-(38-{[(5S)-5-{[(tert-butoxy)carbonyl]amino}-5-{[(1S)-1-{[(1 S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioate) in THF (6 mL) was stirred at room temperature, then lithium hydroxide monohydrate (8.87 mg, 0.211 mmol) in water (3 mL) was added. The reaction solution turned yellow and was stirred for another 2h to achieve complete hydrolysis. The solution was acidified with TFA to pH 6.0, then concentrated under reduced pressure to remove THF, and the residue in water was purified by reverse phase flash chromatography (0.01% TFA) to yield a mixture of compounds 87-9a/87-9b (250 mg, 0.136 mmol, 67.80%, of 1,4-disubstitued triazole isomer 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1 H-1,2,3-triazol-4-yl]methyl}propanedioic acid and 1,5-disubstitued triazole isomer 2-{[1-(38-{[(5S)-5-{[(tert-butoxy)carbonyl]amino}-5-{[(1S)-1-{[(1 S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioic acid) as a white solid. About 30%-44% isomer 1 at retention time 1.792 min, 40%-52% isomer 2 at retention time 1.811 min, ESI m/z=613.3 (M/3+H)⁺; 919.1 (M/2+H)⁺. 1H NMR (400 MHz, DMSO-d6): δ 10.05 (s, 1H), 8.19 (d, J=7.2 Hz, 1H), 8.07-8.03 (m, 1H), 7.82-7.76 (m, 3H), 7.61 (d, J=7.2 Hz, 2H), 7.54 (d, J=8.8 Hz, 1H), 7.37 (d, J=8.8 Hz, 2H), 7.33 (s, 0.5H), 7.32 (s, 0.5H), 7.01 (d, J=8.0 Hz, 1H), 6.54-6.53 (m, 1H), 5.99 (s, 1H), 5.46-5.39 (m, 3H), 5.34-5.23 (m, 3H), 5.09 (s, 2H), 4.48-4.46 (m, 2H), 4.43-4.37 (m, 1H), 4.28-4.23 (m, 1H), 3.91-3.86 (m, 1H), 3.78 (t, J=5.6 Hz, 1H), 3.65-3.56 (m, 4H), 3.51-3.45 (m, 46H), 3.15-2.90 (m, 8H), 2.38 (s, 3H), 2.29 (d, J=6.4 Hz, 2H), 2.23-2.11 (m, 2H), 1.99-1.82 (m, 3H), 1.72-1.49 (m, 3H), 1.49-1.22 (m, 17H), 0.90-0.81 (m, 9H) ppm.

### Step 6

To a round-bottomed flask was added a mixture of compounds 87-9a/87-9b (240 mg, 0.131 mmol, of 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1 H-1,2,3-triazol-4-yl]methyl}propanedioic acid and 2-{[1-(38-{[(5S)-5-{[(tert-butoxy)carbonyl]amino}-5-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamoyl}-3,6,9,12,15,18,21,24,27,30,33,36-dodecaoxaoctatriacontan-1-yl)-1H-1,2,3-triazol-5-yl]methyl}propanedioic acid), followed by a solution of compound 87-10 ((2R,3R,4R,5S)-6-aminohexane-1,2,3,4,5-pentol (87-10, 94.69 mg, 0.523 mmol)) and DIPEA (50.56 mg, 0.392 mmol) in anhydrous DMF (1.5 mL). The brown solution was stirred at room temperature for 5 min, then a solution of HATU (99.35 mg, 0.261 mmol) in anhydrous DMF (1.5 mL) was added dropwise. After addition, the resulting solution was stirred for another 2 hr until LCMS indicated complete conversion. The reaction solution was purified directly by reverse phase flash chromatography (0.01% TFA) to yield a mixture of 1,4-disubstituted triazole and 1,5-disubstituted triazole, which was further purified by Prep-HPLC (0.01% TFA) to yield compound 87-11a (1,4-disubstituted isomer tert-butyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[4-(2-{[(2S,3R,4R,5R)-2,3,4,5,6-pentahydroxyhexyl]carbamoyl}ethyl)-1 H-1,2,3-triazol-1-yl]-3,6,9,12,15,18,21,24,27,30,33,36-dodecaoxanonatriacontan-39-amido}pentyl]carbamate (87-11a, 65 mg, 0.033 mmol, 25.43%)) as a pale yellow solid, and then compound 87-11b (1,5-disubstituted isomer tert-butyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[5-(2-{[(2S,3R,4R,5R)-2,3,4,5,6-pentahydroxyhexyl]carbamoyl}ethyl)-1 H-1,2,3-triazol-1-yl]-3,6,9,12,15,18,21,24,27,30,33,36-dodecaoxanonatriacontan-39-amido}pentyl]carbamate (87-11b, 35 mg, 0.018 mmol, 13.66%)) as a pale yellow solid. 87-11a (majority): ESI m/z: 653.0 (M/3+H)⁺, 979.2 (M/2+H)⁺. ¹H NMR: (400 MHz, DMSO-d6) δ 10.04 (s, 1H), 8.19 (d, J=6.8 Hz, 1H), 8.06 (d, J=7.6 Hz, 1H), 7.86-7.77 (m, 4H), 7.60 (d, J=8.8 Hz, 2H), 7.54 (d, J=8.4 Hz, 1H), 7.36 (d, J=8.4 Hz, 2H), 7.32 (s, 1H), 7.00 (d, J=8.0 Hz, 1H), 6.53 (s, 1H), 5.99 (s, 1H), 5.45-5.35 (m, 3H), 5.35-5.24 (m, 3H), 5.08 (s, 2H), 4.47-4.44 (m, 2H), 4.41-4.36 (m, 1H), 4.27-4.23 (m, 1H), 3.93-3.86 (m, 1H), 3.79 (t, J=4.2 Hz, 2H), 3.67-3.56 (m, 8H), 3.56-3.46 (m, 44H), 3.16-2.90 (m, 12H), 2.83 (t, J=7.6 Hz, 2H), 2.43 (t, J=8.0 Hz, 2H), 2.38 (s, 3H), 2.29 (t, J=6.4 Hz, 2H), 2.23-2.11 (m, 2H), 2.00-1.82 (m, 4H), 1.73-1.57 (m, 3H), 1.50-1.24 (m, 18H), 0.90-0.81 (m, 9H) ppm. One proton of carboxyl group on TFA was revealed.

### 87-11b (Minority):

ESI m/z: 653.0 (M/3+H)⁺; 979.1 (M/2+H)⁺. ¹H NMR: (400 MHz, DMSO-d6) δ10.04 (s, 1H), 8.18 (d, J=7.6 Hz, 1H), 8.04 (d, J=8.8 Hz, 1H), 7.83-7.75 (m, 4H), 7.61 (d, J=8.8 Hz, 2H), 7.54 (d, J=8.8 Hz, 1H), 7.36 (d, J=8.8 Hz, 2H), 7.33 (s, 1H), 7.00 (d, J=8.4 Hz, 1H), 6.53 (s, 1H), 5.99 (s, 1H), 5.45-5.39 (m, 3H), 5.34-5.23 (m, 3H), 5.09 (s, 2H), 4.46 (t, J=4.2 Hz, 2H), 4.41-4.36 (m, 1H), 4.27-4.23 (m, 1H), 3.93-3.88 (m, 1H), 3.79 (t, J=4.6 Hz, 2H), 3.67-3.56 (m, 8H), 3.56-3.47 (m, 44H), 3.13-2.90 (m, 12H), 2.83 (t, J=7.6 Hz, 2H), 2.43 (t, J=7.6 Hz, 2H), 2.38 (s, 3H), 2.29 (t, J=6.4 Hz, 2H), 2.22-2.10 (m, 2H), 2.00-1.85 (m, 4H), 1.73-1.55 (m, 3H), 1.48-1.24 (m, 18H), 0.90-0.81 (m, 9H) ppm. One proton of carboxyl group on TFA was revealed.

### Step 7

Compound 87-11a (tert-butyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-{1-[4-(2-{[(2S,3R,4R,5R)-2,3,4,5,6-pentahydroxyhexyl]carbamoyl}ethyl)-1 H-1,2,3-triazol-1-yl]-3,6,9,12,15,18,21,24,27,30,33,36-dodecaoxanonatriacontan-39-amido}pentyl]carbamate (87-11a, 60 mg, 0.031 mmol)) in methanol (2 mL) was stirred at room temperature, then 2M HCl in methanol (1 mL) was added. The solution was stirred for 1 h until LCMS showed that all starting material was consumed. Then the reaction solution was neutralized with aq. sodium bicarbonate solution, and solvent methanol was evaporated off with a rotary evaporator, and the residue was dissolved in water and purified by reverse phase flash chromatography (0.01% TFA) to collect the desired fractions, which were freeze-dried to yield product 87-12a ({4-[(2S)-2-[(2S)-2-[(2S)-2-amino-6-{1-[5-(2-{[(2S,3R,4R,5R)-2,3,4,5,6-pentahydroxyhexyl]carbamoyl}ethyl)-1 H-1,2,3-triazol-1-yl]-3,6,9,12,15,18,21,24,27,30,33,36-dodecaoxanonatriacontan-39-amido}hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (87-12a, 20 mg, 0.011 mmol)) as a white solid. ESI m/z: 928.7 (M/2+H)⁺, 619.5 (M/3+H)⁺. ¹H NMR: (400 MHz, DMSO-d6) δ 10.09 (s, 1H), 8.41 (d, J=8.4 Hz, 1H), 8.31 (d, J=8.0 Hz, 1H), 8.08-8.02 (m, 4H), 7.85-7.80 (m, 3H), 7.79 (s, 1H), 7.60 (d, J=8.8 Hz, 2H), 7.37 (d, J=8.4 Hz, 2H), 7.32 (s, 1H), 6.54 (s, 1H), 6.00 (t, J=4.0 Hz, 1H), 5.46 (brs, 4H), 5.34-5.25 (m, 3H), 5.09 (s, 2H), 4.47-4.26 (m, 7H), 3.89-3.84 (m, 1H), 3.80-3.76 (m, 2H), 3.60-3.56 (m, 5H), 3.50-3.47 (m, 44H), 3.29-3.22 (m, 2H), 3.16-2.94 (m, 9H), 2.85-2.81 (m, 1H), 2.45-2.41 (m, 1H), 2.39 (s, 3H), 2.31 (t, J=6.4 Hz, 2H), 2.24-2.11 (m, 4H), 2.02-1.98 (m, 1H), 1.90-1.82 (m, 3H), 1.69-1.64 (m, 3H), 1.60-1.55 (m, 1H), 1.46-1.23 (m, 7H), 0.92-0.86 (m, 9H) ppm. One proton of carboxyl group on TFA was revealed.

### Step 8

A solution of compound 87-12a ({4-[(2S)-2-[(2S)-2-[(2S)-2-amino-6-{1-[4-(2-{[(2S,3R,4R,5R)-2,3,4,5,6-pentahydroxyhexyl]carbamoyl}ethyl)-1 H-1,2,3-triazol-1-yl]-3,6,9,12,15,18,21,24,27,30,33,36-dodecaoxanonatriacontan-39-amido}hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (87-12a, 20 mg, 0.011 mmol)) and DIPEA (1.94 mg, 0.015 mmol) in anhydrous DMF (1 mL) was stirred at room temperature for 5 min, then a solution of compound 87-13 (2,5-dioxopyrrolidin-1-yl 6-(2,5-dioxo-2,5-dihydro-1H-pyrrol-1-yl)hexanoate (87-13, 3.08 mg, 0.010 mmol)) in anhydrous DMF (1 mL) was added dropwise by syringe. The resulting solution was stirred for another 4h until LCMS indicated all starting activated ester was consumed and little starting amine was remained. The resulting solution was then acidified to pH 4-5 with TFA, purified by Prep-HPLC (0.01% TFA) to yield desired fractions, which was freeze-dried to yield PB087 ({4-[(2S)-5-(carbamoylamino)-2-[(2S)-2-[(2S)-2-[6-(2,5-dioxo-2,5-dihydro-1 H-pyrrol-1-yl)hexanamido]-6-{1-[4-(2-{[(2S,3R,4R,5R)-2,3,4,5,6-pentahydroxyhexyl]carbamoyl}ethyl)-1 H-1,2,3-triazol-1-yl]-3,6,9,12,15,18,21,24,27,30,33,36-dodecaoxanonatriacontan-39-amido}hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB087, 12 mg, 0.006 mmol, 58.56%)) as a white solid. ESI m/z: 1047.7 (M/2+Na)*, 684.1(M/3+H)⁺, 513.4 (M/4+H)⁺, retention time 6.372 min (HPLC). ¹H NMR: (400 MHz, DMSO-d6) δ 10.04 (s, 1H), 8.13 (d, J=6.8 Hz, 1H), 8.09-8.05 (m, 1H), 7.97 (d, J=8.0 Hz, 1H), 7.86-7.76 (m, 4H), 7.69-7.66 (m, 1H), 7.60 (d, J=8.4 Hz, 2H), 7.36 (d, J=8.4 Hz, 2H), 7.32 (s, 1H), 6.99 (s, 2H), 6.55 (s, 1H), 6.05-5.95 (m, 1H), 5.45 (s, 4H), 5.33-5.24 (m, 3H), 5.08 (s, 2H), 4.47-4.33 (m, 4H), 4.27-4.16 (m, 3H), 3.80-3.75 (m, 2H), 3.67-3.55 (m, 12H), 3.50-3.48 (m, 44H), 3.37-3.34 (m, 2H), 3.29-3.22 (m, 2H), 3.13-2.90 (m, 8H), 2.83 (t, J=7.6 Hz, 1H), 2.43 (d, J=7.60 Hz, 1H), 2.38 (s, 3H), 2.29 (t, J=6.4 Hz, 2H), 2.22-2.05 (m, 4H), 2.00-1.83 (m, 3H), 1.73-1.57 (m, 3H), 1.51-1.32 (m, 9H), 1.24-1.14 (m, 4H), 0.90-0.81 (m, 9H)ppm. One proton of carboxyl group on TFA was revealed.

### Example 19: Preparation of a Drug-Linker Containing a PEG Unit and a Cleavable Linker Attached to Exatecan (PB088)

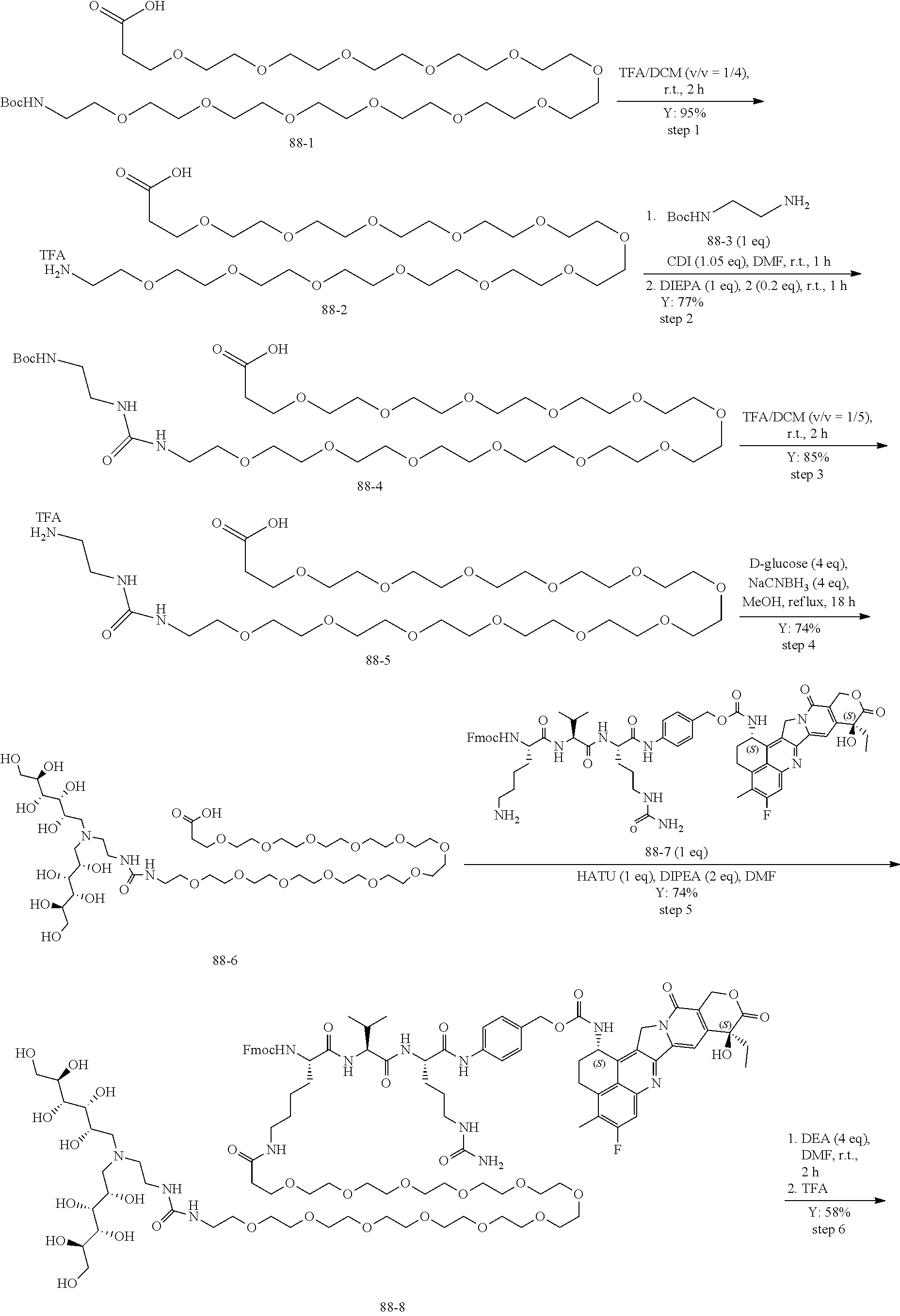

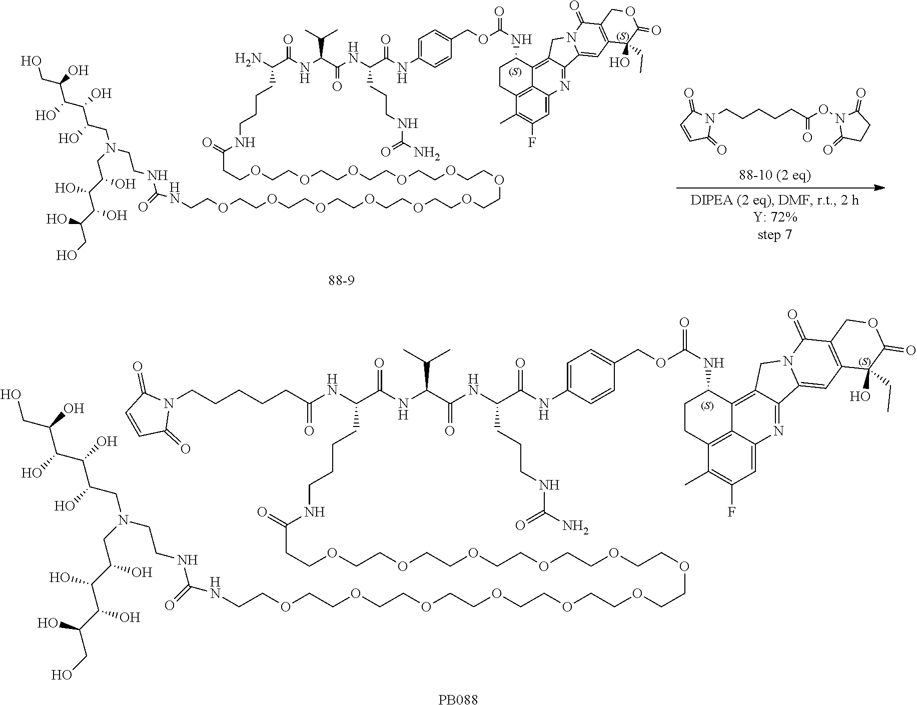

A Drug-Linker containing a PEG unit and a cleavable linker attached to exatecan (PB088) was prepared as follows:

### Step 1

To a solution of compound 88-1 (1-{[(tert-butoxy)carbonyl]amino}-3,6,9,12,15,18,21,24,27,30,33,36-dodecaoxanonatriacontan-39-oic acid (88-1, 500 mg, 0.697 mmol) in DCM (4 mL)) was added TFA (1 mL, 13.463 mmol). The mixture was stirred at room temperature for 2 hours. Then the solution was concentrated and the crude mixture was purified by reverse phase flash chromatography (0.01% TFA) to yield compound 88-2 (1-amino-3,6,9,12,15,18,21,24,27,30,33,36-dodecaoxanonatriacontan-39-oic acid (88-2, 420 mg, 0.680 mmol, 97.59%)) as colorless oil. ESI m/z: 618.4(M+H)⁺.

### Step 2

A solution of compound 88-3 (tert-butyl N-(2-aminoethyl)carbamate (88-3, 0.335 mL, 2.122 mmol)) and CDI (0.264 mL, 2.121 mmol) in DMF (3 mL) was stirred at room temperature for 1h to prepare activated intermediate, then compound 88-2 (1-amino-3,6,9,12,15,18,21,24,27,30,33,36-dodecaoxanonatriacontan-39-oic acid (88-2, 236 mg, 0.382 mmol)) and DIPEA (197.48 mg, 1.528 mmol) were added. The resulting solution was stirred for another 1 h until all starting materials were consumed. The reaction mixture was purified by reverse phase flash chromatography (0.01% TFA) to yield the desired fractions, which weres freeze-dried to yield compound 88-4 (1-{[(2-{[(tert-butoxy)carbonyl]amino}ethyl)carbamoyl]amino}-3,6,9,12,15,18,21,24,27,30,33,36-dodecaoxanonatriacontan-39-oic acid (88-4, 230 mg, 0.286 mmol, 74.89%)) as a colorless oil. ESI m/z: =804.5(M+H)⁺. ¹H NMR (400 MHz, DMSO) b 12.17 (s, 1H), 6.78 (t, J=4.8 Hz, 1H), 6.08-5.81 (m, 2H), 3.60 (t, J=6.4 Hz, 2H), 3.54-3.48 (m, 44H), 3.37 (t, J=5.6 Hz, 3H), 3.17-3.12 (m, 2H), 3.05-2.96 (m, 2H), 2.93-2.87 (m, 2H), 2.44 (t, J=6.4 Hz, 2H), 1.37 (s, 9H) ppm.

### Step 3

To a solution of compound 88-4 (1-{[(2-{[(tert-butoxy)carbonyl]amino}ethyl)carbamoyl]amino}-3,6,9,12,15,18,21,24,27,30,33,36-dodecaoxanonatriacontan-39-oic acid (88-4, 430 mg, 0.535 mmol)) in DCM (5 mL) was added TFA (1 mL, 13.463 mmol). The mixture was stirred at room temperature for 2 hours. Then the solution was concentrated and the crude material was purified by reverse phase flash chromatography (0.01% TFA) to yield a TFA salt of compound 88-5 (1-{[(2-aminoethyl)carbamoyl]amino}-3,6,9,12,15,18,21,24,27,30,33,36-dodecaoxanonatriacontan-39-oic acid (88-5, 320 mg, 0.455 mmol, 85.00%)) as a colorless oil. ESI m/z=704.5 (M+H)⁺.

### Step 4

To a solution of compound 88-5 (1-{[(2-aminoethyl)carbamoyl]amino}-3,6,9,12,15,18,21,24,27,30,33,36-dodecaoxanonatriacontan-39-oic acid (88-5, 163 mg, 0.232 mmol)) in MeOH (20 mL) was added D-glucose (166.90 mg, 0.926 mmol) in portions, and the mixture was heated to 85° C. with stirring for 30 minutes under a N₂ atmosphere. Then NaCNBH₃ (58.21 mg, 0.926 mmol) was added. The reaction mixture was stirred with heating for 18h to complete the reaction. Then the reaction solution was concentrated to dryness and purified by reverse phase flash chromatography (0.01% TFA) to yield the desired fractions, which were freeze-dried to yield compound 88-6 (1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-oic acid (88-6, 200 mg, 0.194 mmol, 83.52%)) as a colorless oil. ESI m/z: 1032.5(M+H)⁺. ¹H NMR (400 MHz, DMSO) b 6.40-6.38 (m, 2H), 4.67 (brs, 8H), 3.70-3.63 (m, 2H), 3.61-3.39 (m, 54H), 3.38-3.37 (m, 6H), 3.22-2.91 (m, 6H), 2.49-2.35 (m, 4H), 2.31 (t, J=6.4 Hz, 2H) ppm. Proton of carboxyl group was not revealed.

### Step 5

A mixture of compound 88-6 (1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-oic acid (88-6, 138.45 mg, 0.134 mmol)), HATU (50.95 mg, 0.134 mmol) and DIPEA (34.61 mg, 0.268 mmol) in DMF (3 mL) was stirred for 15 minutes at room temperature under N₂. Then compound 88-7 ((9H-fluoren-9-yl)methyl N-[(1S)-5-amino-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (88-7, 160 mg, 0.134 mmol)) was added slowly, and the reaction mixture was stirred for 2h. After completion of the reaction (monitored by LCMS), the reaction solution was purified by prep-HPLC (0.01% TFA) to afford compound 88-8 ({4-[(2S)-2-[(2S)-2-[(2S)-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)-2-({[(9H-fluoren-9-yl)methoxy]carbonyl}amino)hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamate (88-8, 220 mg, 0.100 mmol, 74.27%)) as a pale yellow solid. ESI m/z: 1103.6 (M/2+H)⁺.

### Step 6

To a solution of compound 88-8 ({4-[(2S)-2-[(2S)-2-[(2S)-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)-2-({[(9H-fluoren-9-yl)methoxy]carbonyl}amino)hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamate (88-8, 230 mg, 0.104 mmol)) in DMF (2 mL) was added diethyl amine (30.51 mg, 0.417 mmol). The mixture was stirred at room temperature for 2 hours, then the solution was concentrated under vacuo to remove most of diethyl amine, and the crude material was purified by reverse phase flash chromatography (0.01% TFA) to yield a TFA salt of compound 88-9 ({4-[(2S)-2-[(2S)-2-[(2S)-2-amino-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (88-9, 150 mg, 0.076 mmol, 72.53%)) as a pale yellow solid. ESI m/z: 992.6 (M/2+H)⁺.

### Step 7

A solution of compound 88-9 ({4-[(2S)-2-[(2S)-2-[(2S)-2-amino-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (88-9, TFA salt, 52 mg, 0.026 mmol)) in DMF (2 mL) was added compound 88-10 (2,5-dioxopyrrolidin-1-yl 6-(2,5-dioxo-2,5-dihydro-1H-pyrrol-1-yl)hexanoate (88-10, 16.17 mg, 0.052 mmol)) and DIPEA (6.78 mg, 0.052 mmol) at room temperature. After addition, the solution was stirred for 2 h. until LCMS showed that the reaction was completed. Then the reaction solution was purified by Prep-HPLC (0.01% TFA) to yield the desired fractions, which were freeze-dried to yield PB088 ({4-[(2S)-2-[(2S)-2-[(2S)-6-(1-{[(2-{bis[(2S,3R,4R,5R)-2,3,4,5,6-pentahydroxyhexyl]amino}ethyl)carbamoyl]amino}-3,6,9,12,15,18,21,24,27,30,33,36-dodecaoxanonatriacontan-39-amido)-2-[6-(2,5-dioxo-2,5-dihydro-1 H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB088, 40 mg, 0.018 mmol, 70.09%)) as a pale yellow solid. ESI m/z: 1089.6 (M/2+H)⁺, 726.3 (M/3+H)⁺, retention time 6.007 min (HPLC). ¹H NMR (400 MHz, DMSO) b 10.02 (s, 1H), 8.67 (s, 1H), 8.11 (d, J=7.2 Hz, 1H), 8.06 (d, J=8.4 Hz, 1H), 7.95 (d, J=8.0 Hz, 1H), 7.79 (dd, J=11.0, 5.7 Hz, 2H), 7.66 (d, J=8.9 Hz, 1H), 7.60 (d, J=8.4 Hz, 2H), 7.36 (d, J=8.4 Hz, 2H), 7.31 (s, 1H), 7.00 (s, 2H), 6.53 (s, 1H), 6. -6.23 (m, 2H), 5.99 (dd, J=9.8, 4.1 Hz, 1H), 5.43 (d, J=12.1 Hz, 4H), 5.35 (s, 2H), 5.29 (s, 3H), 5.07 (s, 2H), 4.75-4.37 (m, 8H), 4.25-4.17 (m, 2H), 4.00 (d, J=7.6 Hz, 2H), 3.69-3.66 (m, 2H), 3.59-3.54 (m, 5H), 3.52-3.45 (m, 54H), 3.23-3.11 (m, 5H), 3.09-2.85 (m, 5H), 2.38 (s, 3H), 2.35-1.79 (m, 11H), 1.72-1.55 (m, 3H), 1.51-1.42 (m, 6H), 1.42-1.10 (m, 9H), 0.89-0.80 (m, 9H) ppm.

### Example 20: Preparation of a Drug-Linker Containing a PEG Unit and a Cleavable Linker Attached to Exatecan (PB089)

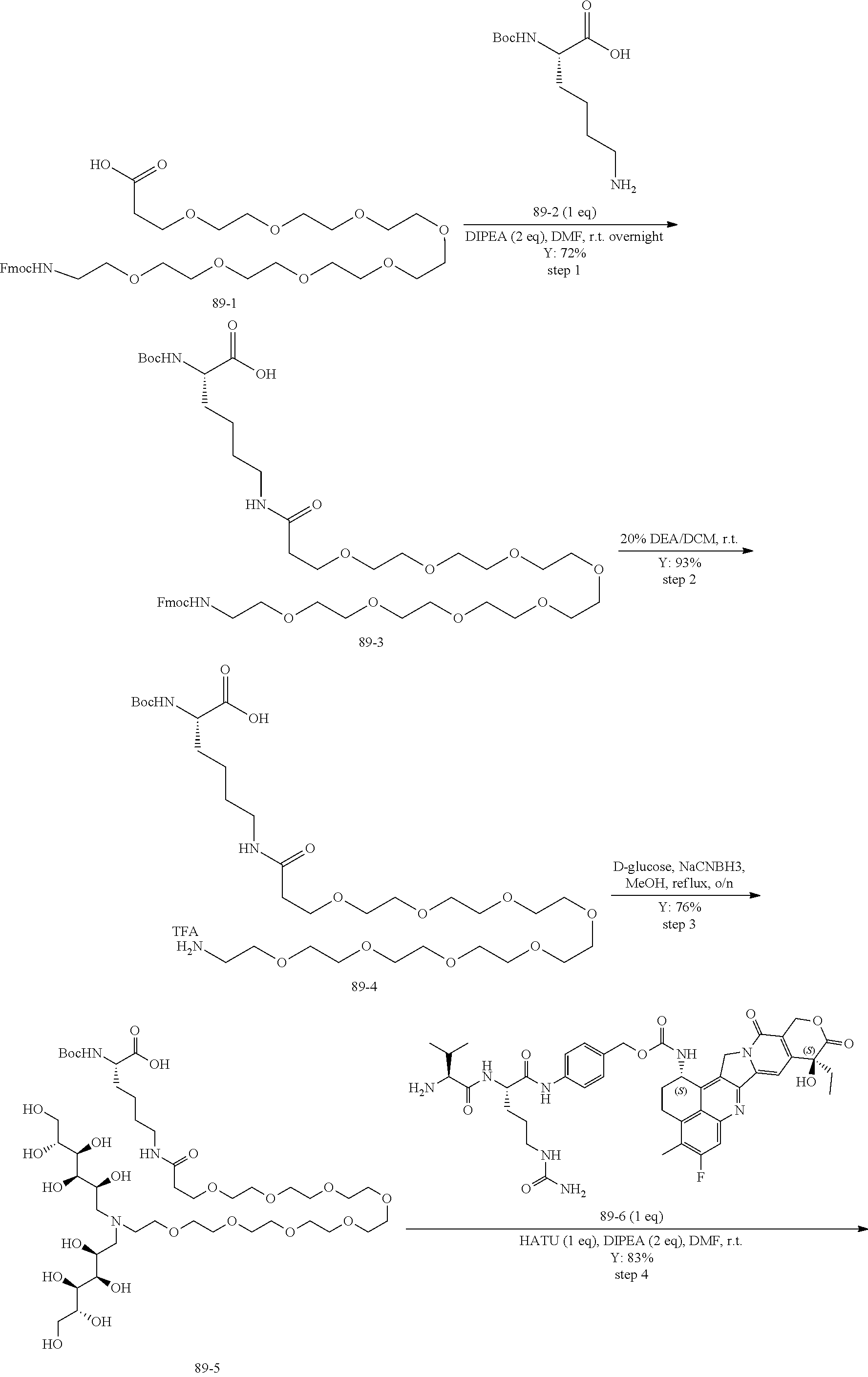

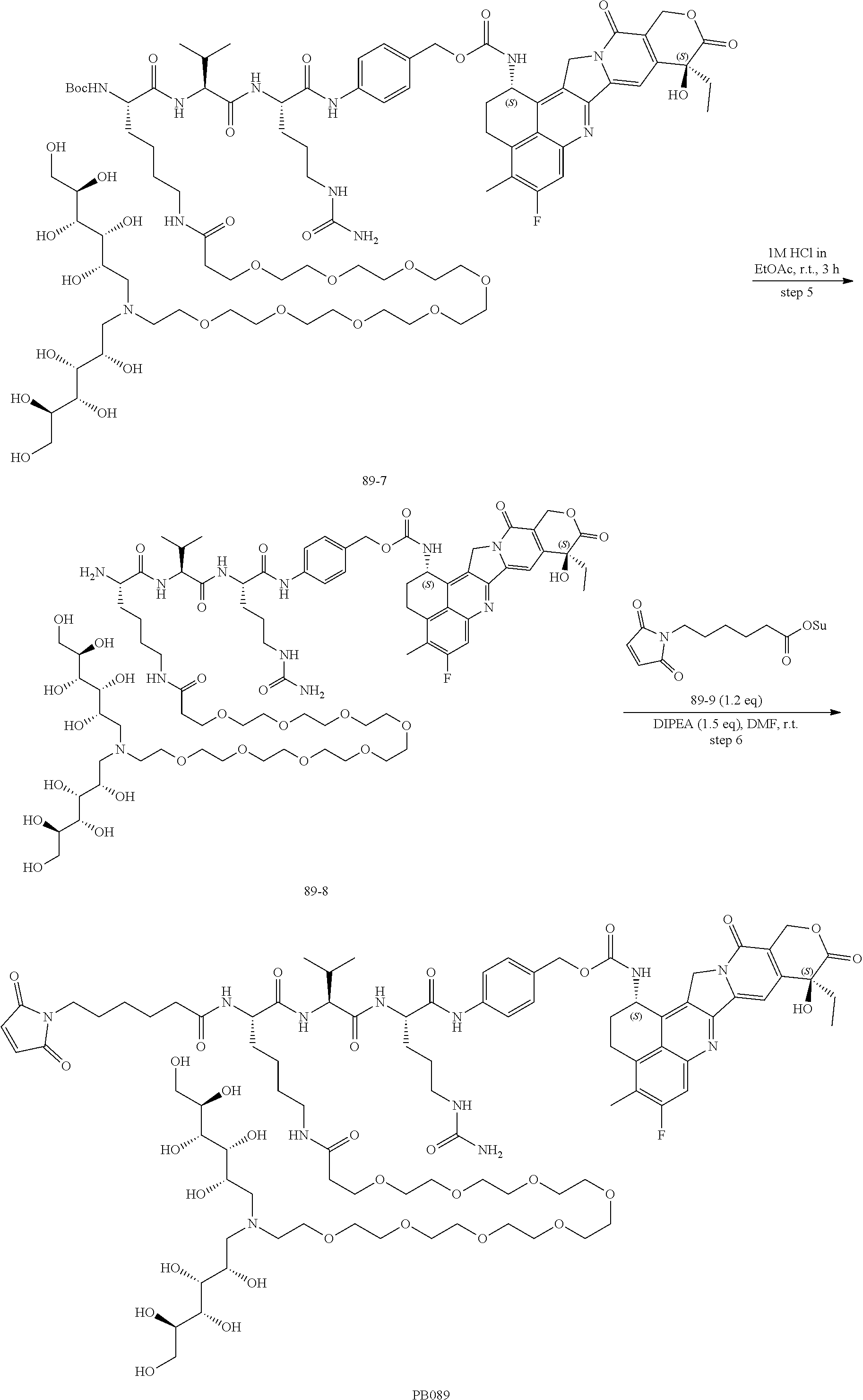

A Drug-Linker containing a PEG unit and a cleavable linker attached to exatecan (PB089) was prepared as follows:

### Step 1

A solution of compound 89-1 (2,5-dioxopyrrolidin-1-yl 1-({[(9H-fluoren-9-yl)methoxy]carbonyl}amino)-3,6,9,12,15,18,21,24-octaoxaheptacosan-27-oate (89-1, 1.83 g, 2.405 mmol)), compound 89-2 ((2S)-6-amino-2-{[(tert-butoxy)carbonyl]amino}hexanoic acid (89-2, 0.59 g, 2.405 mmol)) and DIPEA (0.62 g, 4.811 mmol) in DMF (10 mL) was stirred at room temperature for 18 h until LCMS indicated complete conversion. Then the reaction solution was purified by reverse phase flash chromatography (0.01% TFA) to yield the desired fractions, which were freeze-dried to yield compound 89-3 ((2S)-2-{[(tert-butoxy)carbonyl]amino}-6-[1-({[(9H-fluoren-9-yl)methoxy]carbonyl}amino)-3,6,9,12,15,18,21,24-octaoxaheptacosan-27-amido]hexanoic acid (89-3, 1.54 g, 1.726 mmol, 71.77%)) as a white solid. ESI m/z: 914.5 (M+Na)⁺, 396.8 (M-100)/2+H)⁺.

### Step 2

To a solution of compound 89-3 ((2S)-2-{[(tert-butoxy)carbonyl]amino}-6-[1-({[(9H-fluoren-9-yl)methoxy]carbonyl}amino)-3,6,9,12,15,18,21,24-octaoxaheptacosan-27-amido]hexanoic acid (89-3, 1.14 g, 1.278 mmol)) in DCM (10 mL) was added diethyl amine (0.401 mL, 5.112 mmol). The reaction mixture was stirred at room temperature for 2 hours to completion. Then the mixture was purified by reverse phase flash chromatography to yield compound 89-4 ((2S)-6-(1-amino-3,6,9,12,15,18,21,24-octaoxaheptacosan-27-amido)-2-{[(tert-butoxy)carbonyl]amino}hexanoic acid (89-4, 800 mg, 1.194 mmol, 93.46%)) as a colorless oil. ESI m/z=670.5 (M+H)⁺, 285.8 ((M-100)/2+H)⁺.

### Step 3

To a solution of compound 89-4 ((2S)-6-(1-amino-3,6,9,12,15,18,21,24-octaoxaheptacosan-27-amido)-2-{[(tert-butoxy)carbonyl]amino}hexanoic acid (89-4, 800 mg, 1.194 mmol)) in MeOH (50 mL) was added D-glucose (860.80 mg, 4.778 mmol) in portions, and the mixture was heated to 85° C. with stirring for 30 minutes under a N₂ atmosphere. Then NaCNBH₃ (300.12 mg, 4.776 mmol) was added. After addition, the reaction mixture was heated under refulx for 18h. Then the reaction solution was concentrated and purified by reverse phase flash chromatography (0.01% TFA) to yield the desired fractions, which were freeze-dried to yield compound 89-5 ((2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanoic acid (89-5, 966 mg, 0.968 mmol, 81.06%)) as a colorless oil. ESI m/z: 998.5 (M+H)⁺, 449.8 (M/2+H)⁺.

### Step 4

A mixture of compound 89-5 ((2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanoic acid (89-5, 275 mg, 0.276 mmol)), HATU (91.67 mg, 0.241 mmol) and DIPEA (59.33 mg, 0.459 mmol) in DMF (5 mL) was stirred for 15 minutes at room temperature. Then compound 89-6 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamate (89-6, 193.07 mg, 0.230 mmol)) was added slowly, and the reaction mixture was stirred for 2h. The reaction solution was purified by Prep-HPLC (0.01% TFA) to afford compound 89-7 ({4-[(2S)-2-[(2S)-2-[(2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamate (89-7, 347 mg, 0.191 mmol, 82.99%)) as a pale yellow solid. ESI m/z: 911.0 (M/2+H)⁺, 607.9 (M/3+H)⁺.

### Step 5

A solution of compound 89-7 ({4-[(2S)-2-[(2S)-2-[(2S)-2-{[(tert-butoxy)carbonyl]amino}-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (89-7, 347 mg, 0.191 mmol)) in 1M HCl in ethyl acetate (219 mg, 6.007 mmol) was stirred at room temperature for 2 h until LCMS showed that the reaction was completed. Then solvent was evaporated with a rotary evaporator and the residue was purified by reverse phase flash chromatography (0.01% TFA) to afford compound 89-8 ({4-[(2S)-2-[(2S)-2-[(2S)-2-amino-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (89-8, 93 mg, 0.054 mmol, 28.36%)) as a white solid. ESI=574.5 (M/3+H)⁺, 861.2 (M/2+H)⁺.

### Step 6

To a solution of compound 89-8 ({4-[(2S)-2-[(2S)-2-[(2S)-2-amino-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (89-8, 93 mg, 0.054 mmol)) in DMF (2 mL) was added DIPEA (10.48 mg, 0.081 mmol) and compound 89-9 (2,5-dioxopyrrolidin-1-yl 6-(2,5-dioxo-2,5-dihydro-1H-pyrrol-1-yl)hexanoate (89-9, 24.99 mg, 0.081 mmol)). After addition, the solution was stirred at room temperature for 1 h until all starting amine was consumed (monitored by LMCS). Then the resulting solution was adjusted to pH 6 and purified by Prep-HPLC (0.01% TFA) to afford a TFA salt of product PB089 ({4-[(2S)-5-(carbamoylamino)-2-[(2S)-2-[(2S)-2-[6-(2,5-dioxo-2,5-dihydro-1 H-pyrrol-1-yl)hexanamido]-6-[(30S,31R,32R,33R)-30,31,32,33,34-pentahydroxy-28-[(2S,3R,4R,5R)-2,3,4,5,6-pentahydroxyhexyl]-4,7,10,13,16,19,22,25-octaoxa-28-azatetratriacontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB089, 55 mg, 0.029 mmol, 53.17%)) as a white solid. ESI m/z: 639.1 (M/3+H)⁺, retention time 5.799 min (HPLC). ¹H NMR (400 MHz, DMSO-d6) δ 10.04 (s, 1H), 8.14-8.12 (m, 2H), 8.07 (d, J=8.4 Hz, 1H), 7.97 (d, J=7.6 Hz, 1H), 7.83 (t, J=5.6 Hz, 1H), 7.76 (d, J=11.2 Hz, 1H), 7.67 (d, J=8.8 Hz, 1H), 7.60 (d, J=8.4 Hz, 2H), 7.36 (d, J=8.4 Hz, 2H), 7.31 (s, 1H), 6.99 (s, 2H), 6.548 (s, 1H), 6.02 (t, J=5.6 Hz, 1H), 5.45 (brs, 6H), 5.33-5.21 (m, 3H), 5.08 (s, 2H), 4.82 (brs, 2H), 4.56 (brs, 4H), 4.40-4.17 (m, 5H), 3.99 (brs, 2H), 3.80-3.76 (m, 2H), 3.69-3.67 (m, 2H), 3.62-3.56 (m, 8H), 3.49-3.45 (m, 28H), 3.36-2.90 (m, 16H), 2.37 (s, 3H), 2.29 (t, J=6.4 Hz, 2H), 2.24-2.08 (m, 4H), 2.00-1.81 (m, 3H), 1.69-1.57 (m, 3H), 1.52-1.42 (m, 6H), 1.36-1.29 (m, 3H), 1.22-1.14 (m, 4H), 0.89-0.81 (m, 9H) ppm.

### Example 21: Preparation of a Drug-Linker Containing a PEG Unit and a Cleavable Linker Attached to Exatecan (PB090)

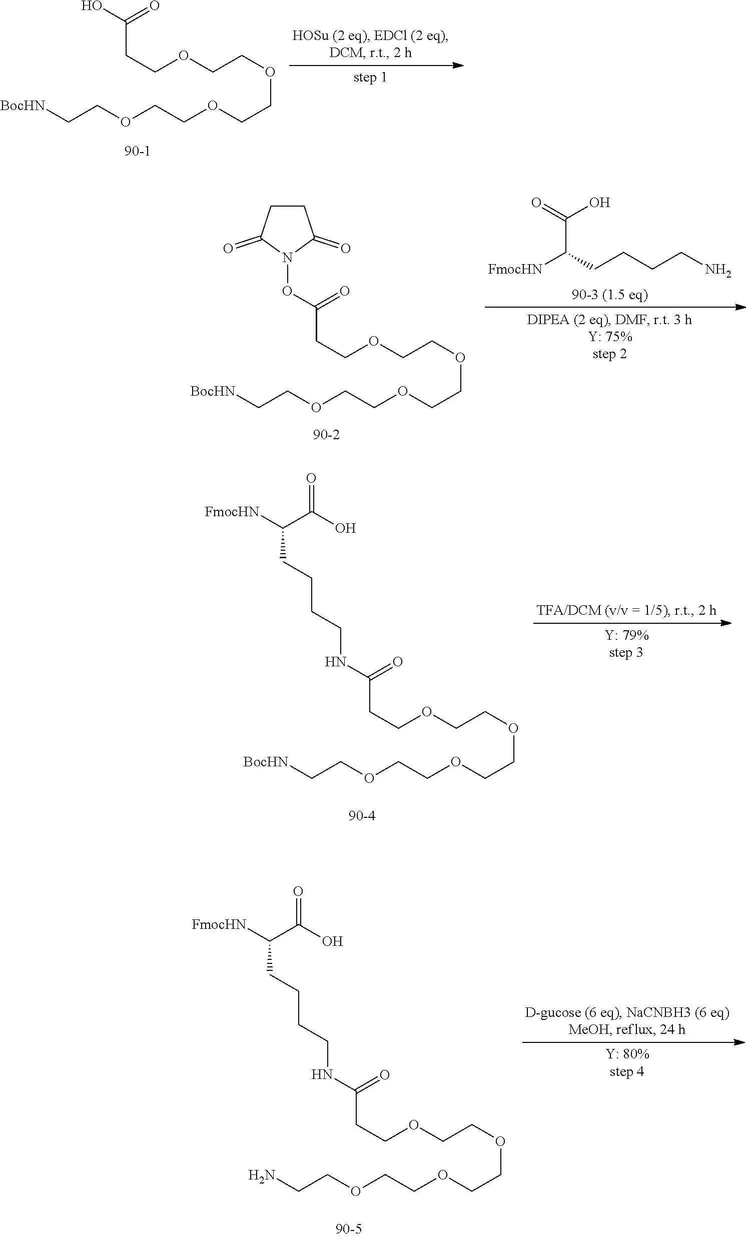

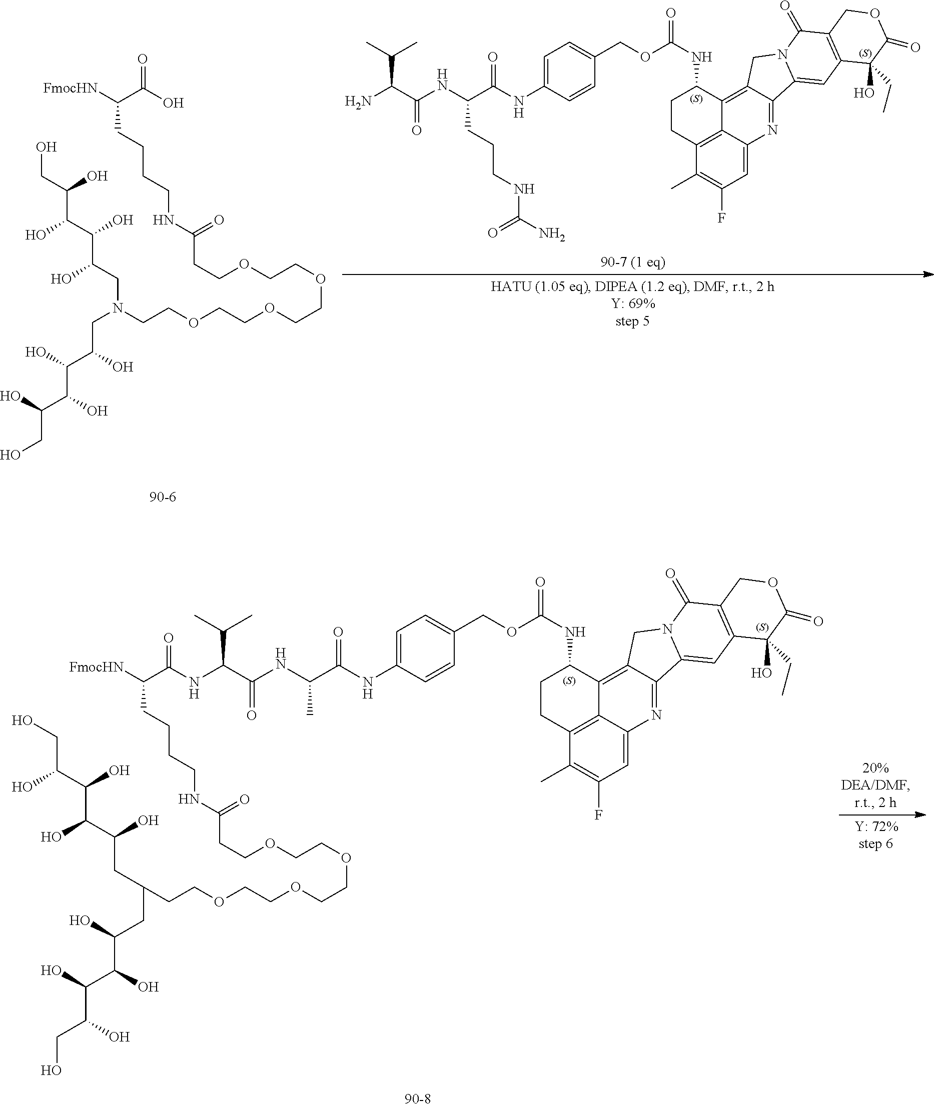

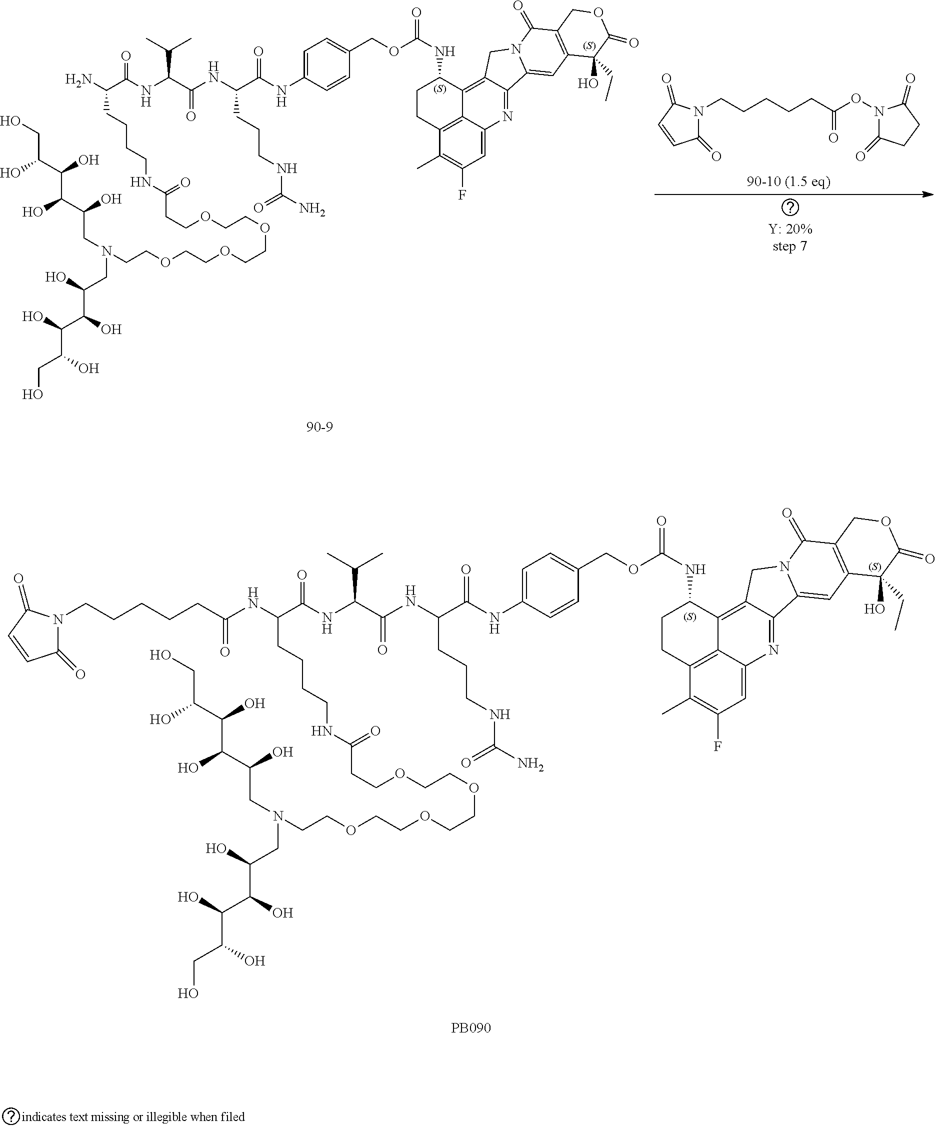

A Drug Linker containing a PEG unit and a cleavable linker attached to exatecan (PB090) was prepared as follows:

### Step 1

To a solution of compound 90-1 (1-{[(tert-butoxy)carbonyl]amino}-3,6,9,12-tetraoxapentadecan-15-oic acid (90-1, 3.737 mL, 11.494 mmol)) in DCM (15 mL) was added HOSu (1.98 g, 17.240 mmol) and EDCl (3.30 g, 17.240 mmol). The mixture was stirred at room temperature for 2h. Then the resulting solution was washed with brine (20 mL) and extracted with DCM (20 mL). The collected organic layer was dried with Na₂SO₄, filtered and the filtrate was evaporated to dryness, affording product 90-2 (2,5-dioxopyrrolidin-1-yl 2,2-dimethyl-4-oxo-3,8,11,14,17-pentaoxa-5-azaicosan-20-oate (90-2, 5.32 g, 11.503 mmol, 100%)). ESI m/z: 485.3 (M+H)⁺.

### Step 2

To a solution of compound 90-2 (2,5-dioxopyrrolidin-1-yl 2,2-dimethyl-4-oxo-3,8,11,14,17-pentaoxa-5-azaicosan-20-oate (90-2, 5.32 g, 11.503 mmol)) in DMF (20 mL) was added compound 90-3 ((2S)-6-amino-2-({[(9H-fluoren-9-yl)methoxy]carbonyl}amino)hexanoic acid (90-3, 6.36 g, 17.254 mmol)) and DIPEA (2.97 g, 23.005 mmol). The mixture was stirred at room temperature for 3h. The resulting solution was purified by reverse phase separation (0.01% TFA) to afford the product 90-4 ((2S)-6-(1-{[(tert-butoxy)carbonyl]amino}-3,6,9,12-tetraoxapentadecan-15-amido)-2-({[(9H-fluoren-9-yl)methoxy]carbonyl}amino)hexanoic acid (90-4, 6.18 g, 8.637 mmol, 75.12%)) as a colorless oil. ESI m/z: 716.5 (M+H)⁺.

### Step 3

To a solution of compound 90-4 ((2S)-6-(1-{[(tert-butoxy)carbonyl]amino}-3,6,9,12-tetraoxapentadecan-15-amido)-2-({[(9H-fluoren-9-yl)methoxy]carbonyl}amino)hexanoic acid (90-4, 6.18 g, 8.633 mmol)) in DCM (20 mL) was added TFA (4 mL, 53.850 mmol). The mixture was stirred at room temperature for 2h. The resulting solution was evaporated and purified by reverse phase flash chromatography (0.01% TFA) to afford the product 90-5 ((2S)-6-(1-amino-3,6,9,12-tetraoxapentadecan-15-amido)-2-({[(9H-fluoren-9-yl)methoxy]carbonyl}amino)hexanoic acid (90-5, 4.2 g, 6.821 mmol, 79.01%)) as colorless oil. ESI m/z: 616.4 (M+H)⁺.

### Step 4

To a solution of compound 90-5 ((2S)-6-(1-amino-3,6,9,12-tetraoxapentadecan-15-amido)-2-({[(9H-fluoren-9-yl)methoxy]carbonyl}amino)hexanoic acid (90-5, 4.2 g, 6.821 mmol)) in MeOH (25 mL) was added D-glucose (7.37 g, 40.926 mmol) and NaBH₃CN (2.57 g, 40.926 mmol). The mixture was stirred at 60° C. for 24h. The resulting solution was concentrated and purified by reverse phase flash chromatography (0.01% TFA) to compound 90-6 (afford the product (2S)-2-({[(9H-fluoren-9-yl)methoxy]carbonyl}amino)-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanoic acid (90-6, 5.16 g, 5.466 mmol, 80.12%)) as a colorless oil. ESI m/z: 945.6 (M+H)⁺.

### Step 5

To a solution of compound 90-6 ((2S)-2-({[(9H-fluoren-9-yl)methoxy]carbonyl}amino)-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanoic acid (90-6, 168 mg, 0.178 mmol)) in DMF (5 mL) was added compound 90-7 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (90-7, 150 mg, 0.178 mmol)), DIPEA (27.58 mg, 0.214 mmol) and HATU (71.05 mg, 0.187 mmol). The mixture was stirred at room temperature for 1.5h. The resulting solution was adjusted to pH 6 and purified by reverse phase flash chromatography (0.01% TFA) to afford the product 90-8 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]pentyl]carbamate (90-8, 217 mg, 0.123 mmol, 69.01%)). ESI m/z: 884.1 (M/2+H)⁺.

### Step 6

To a solution of compound 90-8 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]pentyl]carbamate (90-8, 217 mg, 0.123 mmol)) in DMF (2 mL) was added diethyl amine (0.4 mL, 2.501 mmol). The mixture was stirred at room temperature for 2h. The resulting solution was adjusted to pH 6 and purified by reverse phase flash chromatography (0.01% TFA) to afford the product 90-9 ({4-[(2S)-2-[(2S)-2-[(2S)-2-amino-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (90-9, 138 mg, 0.089 mmol, 72.74%)) as a white solid. ESI m/z: 773.5(M/2+H)⁺.

### Step 7

To a solution of compound 90-9 ({4-[(2S)-2-[(2S)-2-[(2S)-2-amino-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (90-9, 138 mg, 0.089 mmol)) in DMF (2 mL) was added DIPEA (17.32 mg, 0.134 mmol) and compound 90-10 (2,5-dioxopyrrolidin-1-yl 6-(2,5-dioxo-2,5-dihydro-1 H-pyrrol-1-yl)hexanoate (90-10, 41.31 mg, 0.134 mmol)). The mixture was stirred at room temperature for 2h. The resulting solution was adjusted to pH 6 and purified by Prep-HPLC (0.01% TFA) to afford TFA salt of product PB090 ({4-[(2S)-5-(carbamoylamino)-2-[(2S)-2-[(2S)-2-[6-(2,5-dioxo-2,5-dihydro-1 H-pyrrol-1-yl)hexanamido]-6-[(18S,19R,20R,21R)-18,19,20,21,22-pentahydroxy-16-[(2S,3R,4R,5R)-2,3,4,5,6-pentahydroxyhexyl]-4,7,10,13-tetraoxa-16-azadocosanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB090, 32 mg, 0.018 mmol, 20.61%)) as a white solid. ESI m/z: 580.5 (M/3+H)⁺, retention time 6.558 min (HPLC). ¹H NMR (400 MHz, DMSO-d6) δ 10.03 (s, 1H), 8.14-8.12 (d, J=6.8 Hz, 2H), 8.08-8.05 (d, J=8.8 Hz, 1H), 7.98-7.96 (d, J=8.0 Hz, 1H), 7.83-7.81 (t, J=5.6 Hz, 1H), 7.78-7.76 (d, J=10.8 Hz, 1H), 7.67-7.64 (d, J=8.0 Hz, 1H), 7.61-7.59 (d, J=8.0 Hz, 2H), 7.37-7.35 (d, J=8.4 Hz, 2H), 7.31 (s, 1H), 7.00 (s, 2H), 6.54 (s, 1H), 6.01 (t, J=5.6 Hz, 1H), 5.45 (brs, 6H), 5.29-5.27 (m, 3H), 5.08 (s, 2H), 4.86-4.74 (m, 2H), 4.65-4.40 (m, 5H), 4.40-4.35 (m, 1H), 4.27-4.17 (m, 2H), 4.04-3.94 (m, 2H), 3.79-3.76 (m, 2H), 3.69-3.67 (m, 2H), 3.67-3.54 (m, 8H), 3.53-3.42 (m, 18H), 3.29-3.27 (m, 4H), 3.20-2.90 (m, 6H), 2.37 (s, 3H), 2.29 (t, J=6.4 Hz, 2H), 2.22-2.08 (m, 4H), 2.02-1.81 (m, 3H), 1.71-1.56 (m, 3H), 1.52-1.42 (m, 6H), 1.40-1.14 (m, 8H), 0.89-0.81 (m, 9H) ppm. One proton of carboxyl group on TFA was revealed.

### Example 22: Preparation of a Drug-Linker Containing a PEG Unit and a Cleavable Linker Attached to Exatecan (PB091)

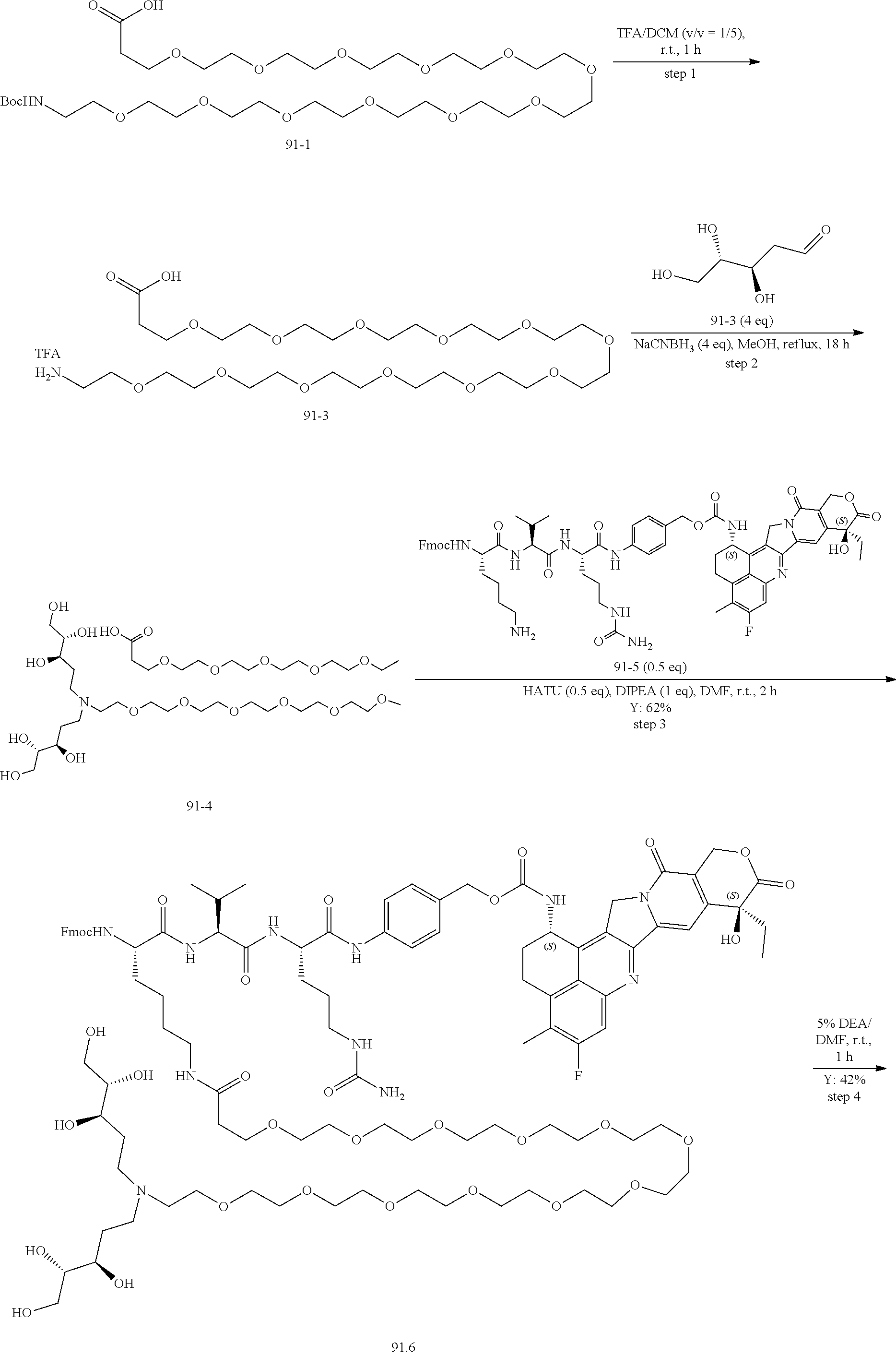

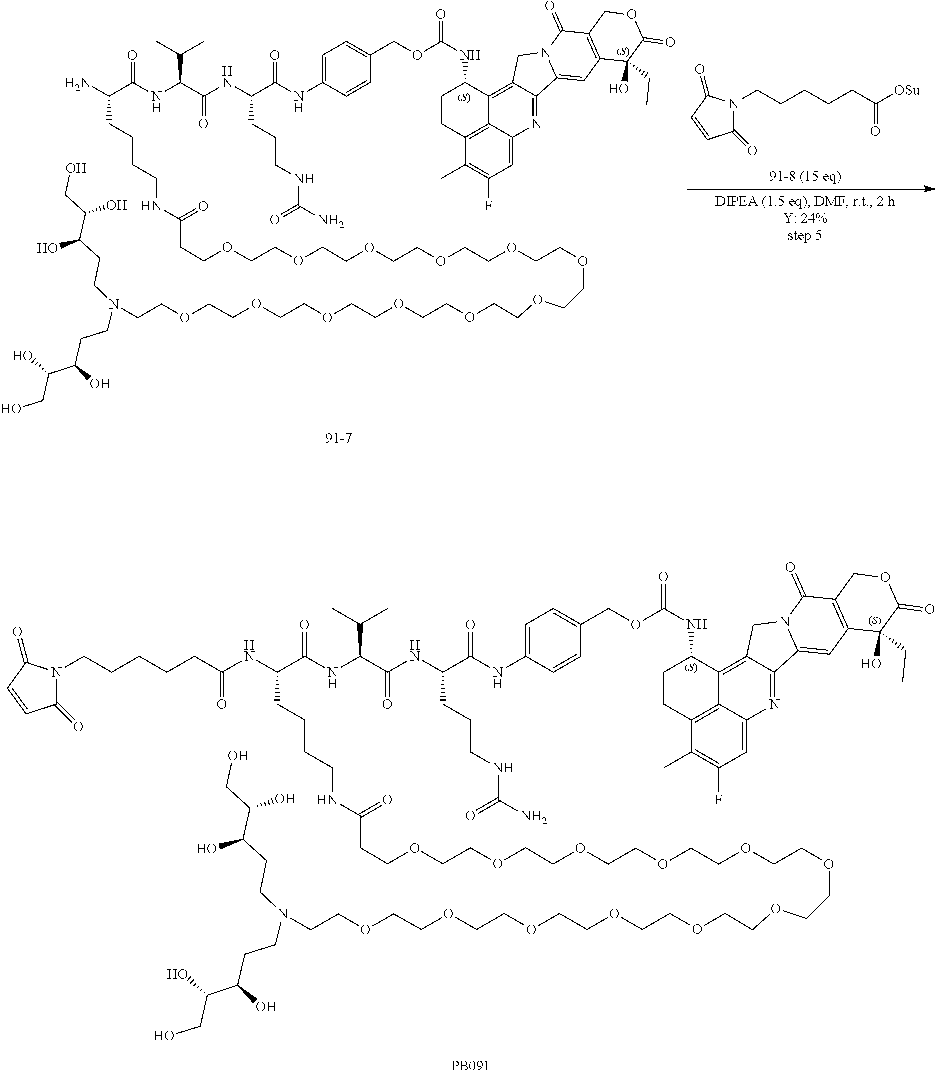

A drug-linker containing a PEG unit and a cleavable linker attached to exatecan (PB091) was prepared as follows:

### Step 1

A solution of compound 91-1 (1-{[(tert-butoxy)carbonyl]amino}-3,6,9,12,15,18,21,24,27,30,33,36-dodecaoxanonatriacontan-39-oic acid (91-1, 560 mg, 0.781 mmol)) in DCM (10 mL) was treated with TFA (2 mL, 26.925 mmol) and stirred at room temperature for 1h until LCMS showed that the reaction was completed. Then the solution was concentrated, and the residue was suspended in DCM and concentrated again to yield crude product 91-2 (1-amino-3,6,9,12,15,18,21,24,27,30,33,36-dodecaoxanonatriacontan-39-oic acid, TFA salt (91-2, 600 mg, 0.821 mmol, 105.09%)) as a white solid. The product was used as such in the next step. ESI m/z=618.5 (M+H)⁺.

### Step 2

A reaction solution of compound 91-2 (1-amino-3,6,9,12,15,18,21,24,27,30,33,36-dodecaoxanonatriacontan-39-oic acid, TFA salt (91-2, 570.91 mg, 0.781 mmol)), compound 91-3 ((3R,4S)-3,4,5-trihydroxypentanal (91-3, 0.395 mL, 3.124 mmol)) and NaCNBH₃ (0.121 mL, 3.124 mmol) in methanol (16 mL) was heated under reflux under N₂for 18 h until the reaction was complete as indicated by LCMS. The solvents were evaporated, and the residue was dissolved in water, and then purified by reverse phase flash chromatography (0.01% TFA) to yield the desired product 91-4 ((43R,44S)-43,44,45-trihydroxy-40-[(3R,4S)-3,4,5-trihydroxypentyl0]-4,7,10,13,16,19,22,25,28,31,34,37-dodecaoxa-40-azapentatetracontanoic acid (91-4, 700 mg, 0.821 mmol, 105.08%)) as a white foam, mixed with excess ribose. ESI m/z: 427.8 (M/2+H)⁺, 854.6 (M+H)⁺.

### Step 3

A solution of compound 91-4 ((43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanoic acid (91-4, 214.68 mg, 0.252 mmol)), compound 91-5 ((9H-fluoren-9-yl)methyl N-[(1S)-5-amino-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (91-5, 150 mg, 0.126 mmol)) and DIPEA (32.47 mg, 0.252 mmol) in anhydrous DMF (2.0 mL) was stirred at room temperature for 5 min, and then a solution of HATU (47.85 mg, 0.126 mmol) in anhydrous DMF (0.5 mL) was added slowly. After addition, the resulting solution was stirred for another 2 h at room temperature until LCMS indicated complete reaction. The reaction solution was purified directly by reverse phase flash chromatography (0.01% TFA) to yield compound 91-6 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]pentyl]carbamate (91-6, 160 mg, 0.079 mmol, 62.72%)) as a white solid. ESI m/z: 507.8 (M/4+H)⁺, 676.7 (M/3+H)⁺.

### Step 4

A solution of compound 91-6 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]pentyl]carbamate (91-6, 80 mg, 0.039 mmol)) in DMF (1.5 mL) was stirred at room temperature and diethyl amine (0.08 mL, 0.777 mmol) was added. The resulting solution was stirred for another 1 h until LCMS showed that the reaction was completed. Diethyl amine was removed under vacuo, and the residue in DMF was purified by Prep-HPLC (10 mM ammonia bicarbonate) to yield the expected product 91-7 ({4-[(2S)-2-[(2S)-2-[(2S)-2-amino-6-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (91-7, 30 mg, 0.017 mmol, 42.12%)) as a white solid. ESI m/z: 903.7 (M/2+H)⁺, 602.7 (M/3+H)⁺. ¹H NMR: (400 MHz, DMSO-d6) δ 10.11 (s, 1H), 8.35-8.31 (m, 5H), 8.09-8.06 (m, 2H), 7.83-7.77 (m, 2H), 7.61 (d, J=8.8 Hz, 2H), 7.36 (d, J=8.4 Hz, 2H), 7.32 (s, 1H), 6.09 (s, 1H), 5.46 (s, 4H), 5.30-5.29 (m, 3H), 5.08 (s, 2H), 4.41-4.34 (m, 1H), 4.27-4.23 (m, 1H), 3.59-3.56 (m, 4H), 3.50-3.47 (m, 44H), 3.47-3.46 (m, 2H), 3.36-3.30 (m, 4H), 3.30-3.22 (m, 4H), 3.14-3.08 (m, 2H), 3.03-2.95 (m, 4H), 2.68-2.56 (m, 6H), 2.38 (s, 3H), 2.29 (t, J=6.4 Hz, 2H), 2.22-2.13 (m, 4H), 2.00-1.84 (m, 4H), 1.76-1.65 (m, 4H), 1.65-1.56 (m, 3H), 1.46-1.28 (m, 10H), 0.90-0.81 (m, 9H) ppm. Two protons of carboxyl group on TFA was revealed between 8.35-8.31 ppm.

### Step 5

To a solution of compound 91-7 ({4-[(2S)-2-[(2S)-2-[(2S)-2-amino-6-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (91-7, 30 mg, 0.017 mmol)) in DMF (2 mL) was added DIPEA (3.30 mg, 0.026 mmol) and compound 91-8 (2,5-dioxopyrrolidin-1-yl 6-(2,5-dioxo-2,5-dihydro-1H-pyrrol-1-yl)hexanoate (91-8, 7.86 mg, 0.026 mmol)). The mixture was stirred at room temperature for 2h. The resulting solution was purified by Prep-HPLC (0.01% TFA) to afford the product PB091 ({4-[(2S)-5-(carbamoylamino)-2-[(2S)-2-[(2S)-2-[6-(2,5-dioxo-2,5-dihydro-1 H-pyrrol-1-yl)hexanamido]-6-[(43R,44S)-43,44,45-trihydroxy-40-[(3R,4S)-3,4,5-trihydroxypentyl]-4,7,10,13,16,19,22,25,28,31,34,37-dodecaoxa-40-azapentatetracontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB091, 7.5 mg, 0.004 mmol, 22.58%)) as a white solid. ESI m/z: 667.0(M/3+H)⁺, 500.8(M/4+H)⁺, retention time 5.791 min (HPLC). ¹H NMR (400 MHz, DMSO-d6) δ 10.03 (s, 1H), 9.10 (brs, 1H), 8.13-8.10 (d, J=7.2 Hz, 1H), 8.07-8.05 (d, J=8.8 Hz, 1H), 7.98-7.96 (d, J=8.0 Hz, 1H), 7.83-7.80 (t, J=5.2 Hz, 1H), 7.77-7.74 (d, J=10.8 Hz, 1H), 7.68-7.65 (d, J=8.4 Hz, 1H), 7.61-7.59 (d, J=8.0 Hz, 2H), 7.38-7.35 (d, J=8.4 Hz, 2H), 7.31 (s, 1H), 6.99 (s, 2H), 6.54 (s, 1H), 6.02-6.01 (m, 1H), 5.45 (brs, 4H), 5.29-5.26 (m, 3H), 5.08 (s, 2H), 4.91 (brs, 2H), 4.76 (brs, 2H), 4.52 (brs, 2H), 4.38-4.36 (m, 1H), 4.25-4.17 (m, 2H), 3.75-3.74 (m, 2H), 3.58-3.52 (m, 6H), 3.51-3.46 (m, 44H), 3.37-2.91 (m, 16H), 2.20 (s, 3H), 2.29 (t, J=6.4 Hz, 2H), 2.21-2.08 (m, 4H), 2.01-1.83 (m, 6H), 1.75-1.59 (m, 6H), 1.48-1.36 (m, 6H), 1.29-1.24 (m, 3H), 1.23-1.16 (m, 4H), 0.89-0.81 (m, 9H) ppm. One proton of carboxyl group on TFA was revealed.

### Example 23: Preparation of a Drug-Linker Containing a PEG Unit and a Cleavable Linker Attached to Exatecan (PB092)

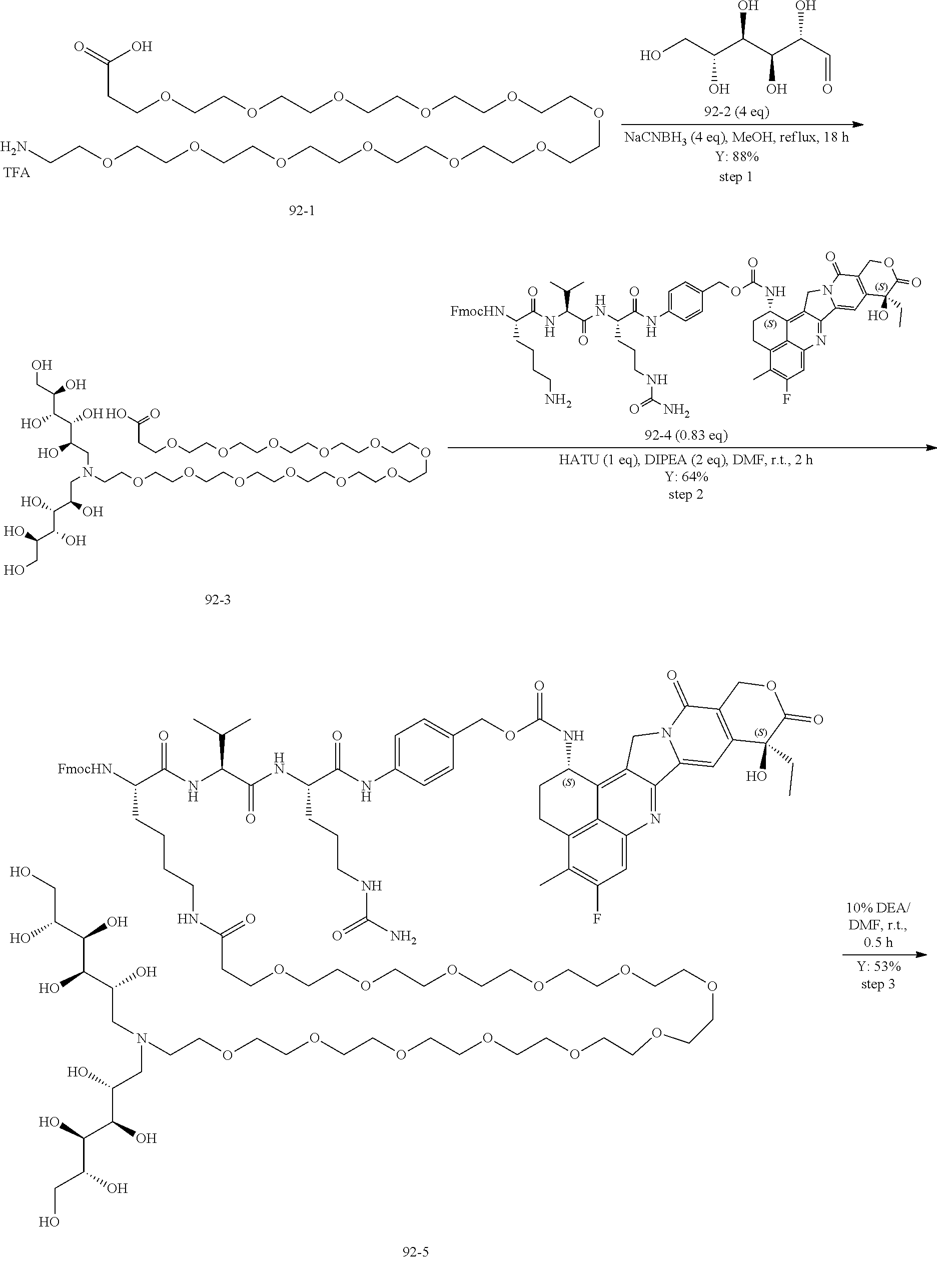

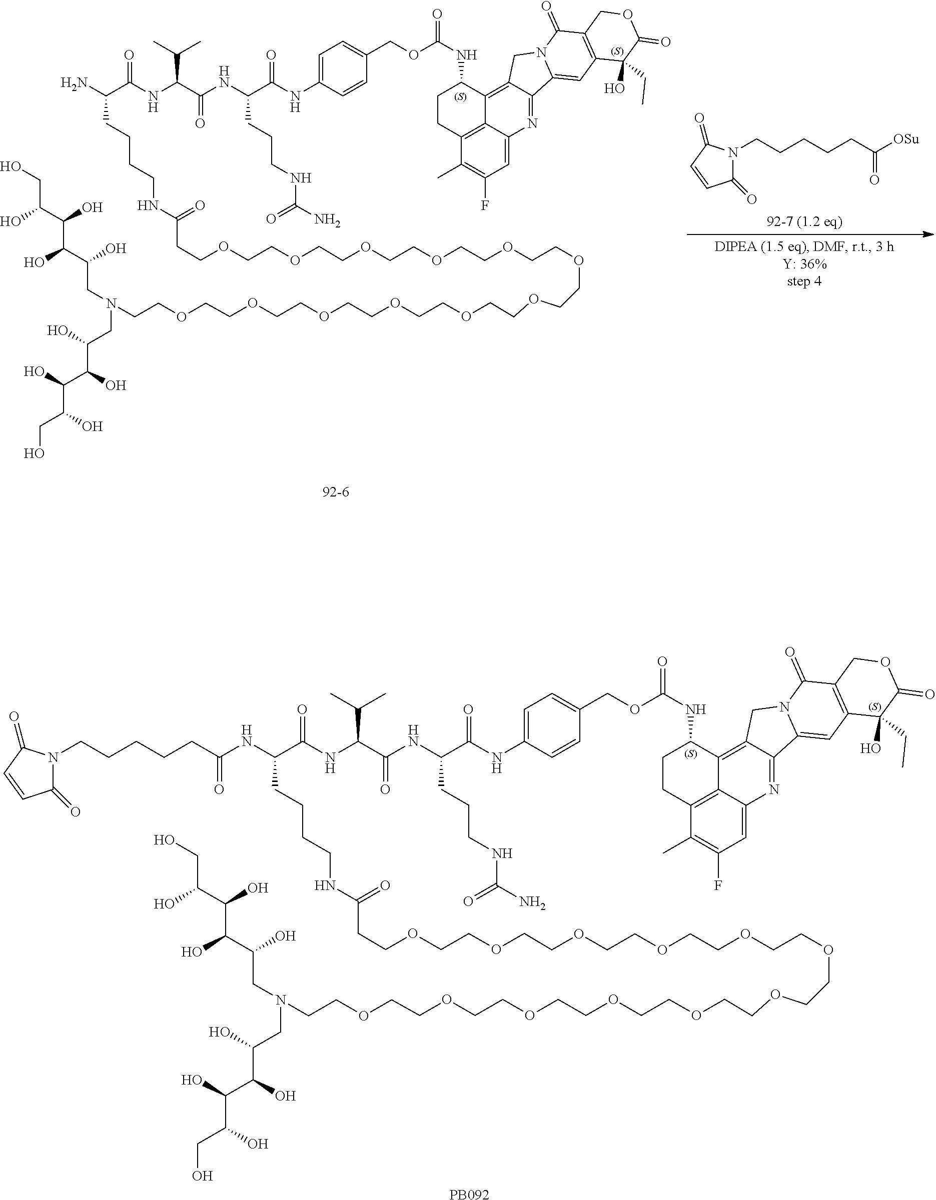

A Drug-Linker containing a PEG unit and a cleavable linker attached to exatecan (PB092) was prepared as follows:

### Step 1

A reaction mixture of compound 92-1 (1-amino-3,6,9,12,15,18,21,24,27,30,33,36-dodecaoxanonatriacontan-39-oic acid, TFA salt (92-1, 570.91 mg, 0.781 mmol)), compound 92-2 ((2S,3S,4R,5R)-2,3,4,5,6-pentahydroxyhexanal (92-2, 562.32 mg, 3.124 mmol)), and Na(CN)BH₃ (193.69 mg, 3.124 mmol) in methanol (16 mL) was heated under reflux under N₂ for 18 h until the reaction was complete as indicated by LCMS. The solvents were evaporated with a rotary evaporator, and the residue was dissolved in water and purified by reverse phase flash chromatography (0.01% TFA) to give the desired product 92-3 ((42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanoic acid (92-3, 650 mg, 0.687 mmol, 87.98%)) as a white foam. ESI m/z: 473.9 (M/2+H)⁺. 946.6 (M+H)⁺.

### Step 2

A solution of compound 92-3 ((42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanoic acid (92-3, 199.95 mg, 0.212 mmol)), compound 92-4 ((9H-fluoren-9-yl)methyl N-[(1S)-5-amino-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (92-4, 210 mg, 0.176 mmol)) and DIPEA (8.02 mg, 0.062 mmol) in anhydrous DMF (4 mL) was stirred at room temperature for 5 min, then a solution of HATU (67.04 mg, 0.176 mmol) in anhydrous DMF (4 mL) was added dropwise over 5 min. The resulting solution was stirred for another 2 h at room temperature until LCMS indicated complete reaction. The reaction solution was purified directly by reverse phase flash chromatography (0.01% TFA) to yield compound 92-5 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]pentyl]carbamate (92-5, 240 mg, 0.113 mmol, 64.22%)) as a white solid. ESI m/z=707.2 (M/2+H)⁺, 530.9 (M/3+H)⁺. ¹H NMR (400 MHz, DMSO-d6): δ 10.06 (s, 1H), 8.16 (d, J=6.4 Hz, 1H), 8.07 (d, J=8.8 Hz, 1H), 7.95 (brs, 1H), 7.88 (d, J=7.6 Hz, 2H), 7.83-7.77 (m, 2H), 7.73-7.70 (m, 3H), 7.60 (d, J=8.0 Hz, 2H), 7.55 (d, J=3.6 Hz, 1H), 7.43-7.31 (m, 7H), 6.54 (s, 1H), 5.99 (t, J=5.6 Hz, 1H), 5.50-5.34 (m, 5H), 5.34-5.24 (m, 3H), 5.08 (s, 2H), 4.67-4.23 (m, 10H), 4.07-3.96 (m, 1H), 3.96-3.83 (m, 2H), 3.83-3.76 (m, 2H), 3.65-3.44 (m, 62H), 3.24-3.11 (m, 4H), 3.11-2.89 (m, 6H), 2.38 (s, 3H), 2.29 (d, J=6.4 Hz, 2H), 2.22-2.10 (m, 2H), 2.00-1.81 (m, 3H), 1.70-1.48 (m, 4H), 1.48-1.24 (m, 7H), 0.90-0.81 (m, 9H) ppm. One proton of carboxyl group on TFA was revealed.

### Step 3

A solution of compound 92-5 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]pentyl]carbamate (92-5, 230 mg, 0.109 mmol)) in DMF (1 mL) was stirred at room temperature. and diethyl amine (0.11 mL, 1.068 mmol) was added. The resulting solution was stirred for 30 min to completion (monitored by LCMS). Then the diethyl amine was evaporated off and the residue in DMF was purified by reverse phase flash chromatography (0.01% TFA) to yield the expected product 92-6 ({4-[(2S)-2-[(2S)-2-[(2S)-2-amino-6-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (92-6, 130 mg, 0.069 mmol, 63.13%)) as a white solid. ESI m/z: 949.6 (M/2+H)⁺, 633.2 (M/3+H)⁺.

### Step 4

A solution of compound 92-6 ({4-[(2S)-2-[(2S)-2-[(2S)-2-amino-6-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (92-6, 50 mg, 0.026 mmol)) and compound 92-7 (2,5-dioxopyrrolidin-1-yl 6-(2,5-dioxo-2,5-dihydro-1H-pyrrol-1-yl)hexanoate (92-7, 9.74 mg, 0.032 mmol)) in anhydrous DMF (1 mL) was stirred at room temperature for 5 min, then DIPEA (5.10 mg, 0.040 mmol) was added. The resulting solution was stirred for another 3 hr until LCMS indicated the starting amine was substantially consumed. Then the resulting solution was purified directly by PrepHPLC (0.01% TFA) to yield a TFA salt of PB092 ({4-[(2S)-5-(carbamoylamino)-2-[(2S)-2-[(2S)-2-[6-(2,5-dioxo-2,5-dihydro-1H-pyrrol-1-yl)hexanamido]-6-[(42R,43R,44R,45R)-42,43,44,45,46-pentahydroxy-40-[(2R,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0\hat{ }{20,24}]tetracosa-1,6(11),12,14,16,18,20(24)-heptaen-23-yl]carbamate (PB092, 20 mg, 0.010 mmol, 36.31%)) as a white solid. ESI m/z: 697.5 (M/3+H)⁺, retention time 5.759 min (HPLC). ¹H NMR (400 MHz, DMSO-d6): δ 10.03 (s, 1H), 8.12 (d, J=7.2 Hz, 1H), 8.06 (d, J=8.8 Hz, 1H), 7.96 (d, J=8.0 Hz, 2H), 7.82-7.77 (m, 2H), 7.66 (d, J=8.8 Hz, 1H), 7.60 (d, J=8.4 Hz, 2H), 7.36 (d, J=8.4 Hz, 2H), 7.32 (s, 1H), 7.00 (s, 2H), 6.54 (s, 1H), 5.99 (t, J=6.0 Hz, 1H), 5.49-5.34 (m, 5H), 5.34-5.24 (m, 3H), 5.08 (s, 2H), 4.72-4.31 (m, 7H), 4.31-4.17 (m, 2H), 3.95-3.87 (m, 2H), 3.81-3.76 (m, 2H), 3.65-3.46 (m, 6H), 3.26-3.16 (m, 4H), 3.11-2.92 (m, 6H), 2.38 (s, 3H), 2.29 (d, J=6.4 Hz, 2H), 2.24-2.07 (m, 4H), 2.00-1.83 (m, 3H), 1.72-1.56 (m, 3H), 1.48-1.41 (m, 6H), 1.36-1.13 (m, 8H), 0.90-0.81 (m, 9H) ppm. One proton of carboxyl group on TFA was revealed.

### Example 24: Preparation of a Drug-Linker Containing a PEG Unit and a Cleavable Linker Attached to Exatecan (PB093)

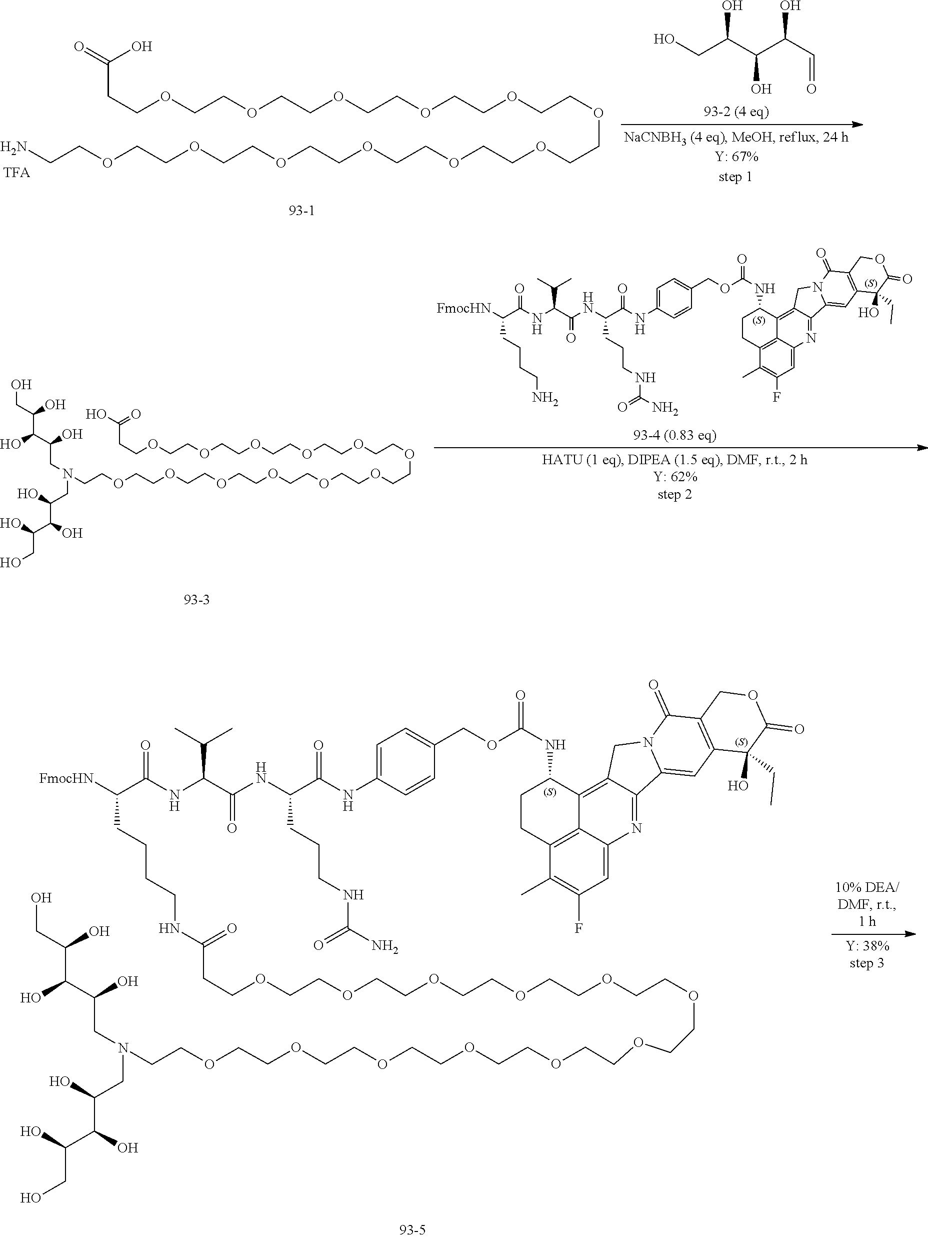

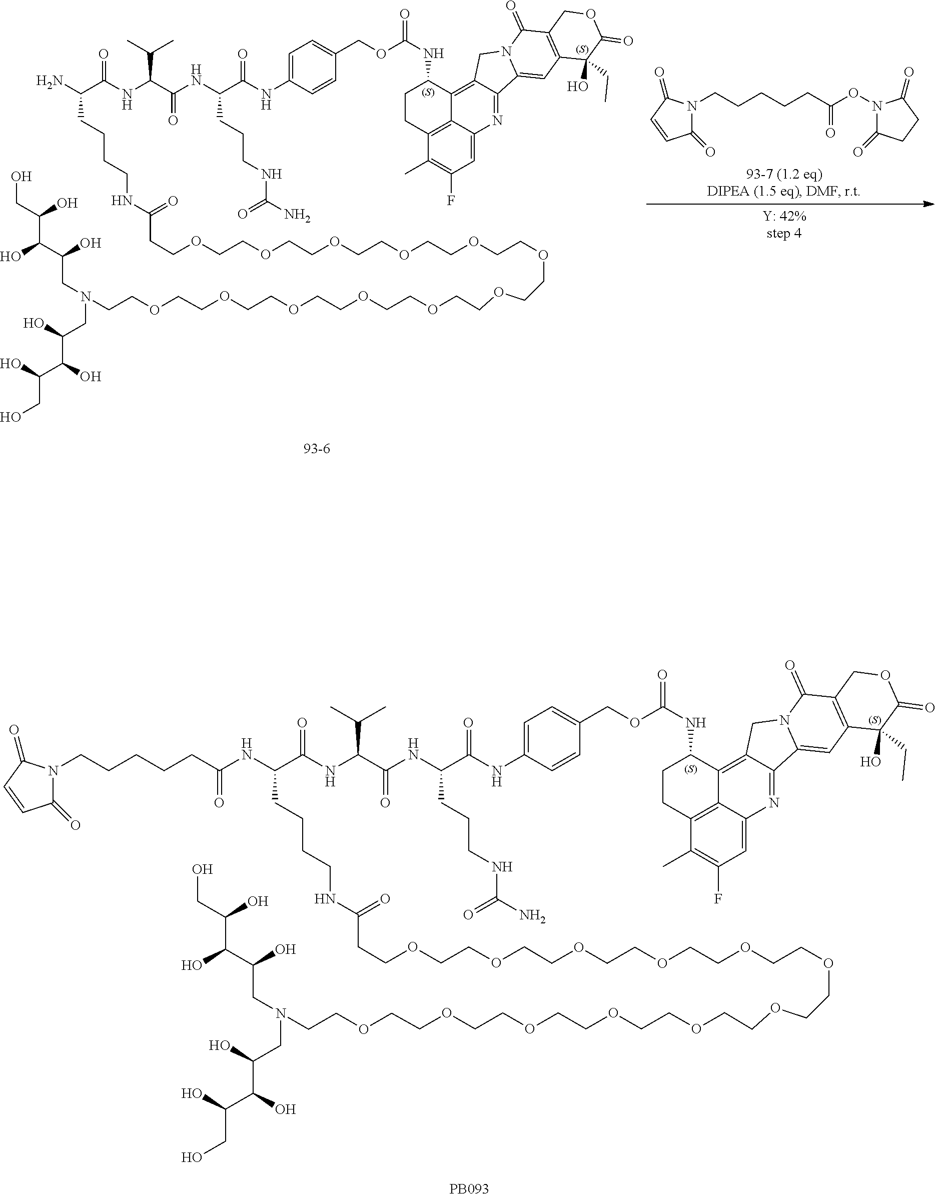

A Drug-Linker containing a PEG unit and a cleavable linker attached to exatecan (PB093) was prepared as follows:

### Step 1

A solution of compound 93-1 (1-amino-3,6,9,12,15,18,21,24,27,30,33,36-dodecaoxanonatriacontan-39-oic acid (93-1, TFA salt, 500 mg, 0.684 mmol)) in MeOH (8 mL) was treated with compound 93-2 ((2R,3S,4R)-2,3,4,5-tetrahydroxypentanal (93-2, 410.75 mg, 2.736 mmol)) and stirred at room temperature for 2h. Then sodium cyanoborohydride (171.93 mg, 2.736 mmol) was added, and the mixture was warmed to 50° C. for 24 h. The solution was concentrated to half volume and purified by reverse phase flash chromatography (0.01% TFA) to yield product 93-3 ((42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanoic acid (93-3, 417 mg, 0.471 mmol, 68.81%)) as a white solid. ESI m/z: 886.6 (M+H)⁺.

### Step 2

To a solution of compound 93-3 ((42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanoic acid (93-3, 200 mg, 0.226 mmol)) in DMF (2 mL) were added compound 93-4 ((9H-fluoren-9-yl)methyl N-[(1S)-5-amino-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (93-4, 228.59 mg, 0.192 mmol)) and DIPEA (43.76 mg, 0.339 mmol), and then a solution of HATU (85.83 mg, 0.226 mmol) in DMF (0.5 mL) was added slowly. After addition, the reaction was stirred at room temperature for another 1 h. The mixture was concentrated and purified by reverse phase flash chromatography (0.01% TFA) to afford compound 93-5 ((9H-fluoren-9-yl)methyl N-[(1S)-1-{[(1 S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]pentyl]carbamate (93-5, 290 mg, 0.141 mmol, 62.38%)) as a white solid. ESI m/z 1030.7 (M/2+H)⁺.

### Step 3

A solution of compound 93-5 ((9H-fluoren-9-yl)methyl N-[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]pentyl]carbamate (93-5, 290 mg, 0.141 mmol)) in DMF (2 mL) was combined with diethyl amine (0.2 mL, 1.250 mmol) and stirred at room temperature for 1 h to completion (monitored by LCMS). Then diethyl amine was removed with a rotary evaporator and the residue was purified by reverse phase flash chromatography (0.01% TFA) to yield product 93-6 ({4-[(2S)-2-[(2S)-2-[(2S)-2-amino-6-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (93-6, 100 mg, 0.054 mmol, 38.65%)) as a white solid. ESI m/z: 919.7 (M/2+H)⁺.

### Step 4

A solution of compound 93-6 ({4-[(2S)-2-[(2S)-2-[(2S)-2-amino-6-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (93-6, 110 mg, 0.060 mmol)) in DMF (1.5 mL) was stirred at room temperature, then DIPEA (0.015 mL, 0.090 mmol) and compound 93-7 (2,5-dioxopyrrolidin-1-yl 6-(2,5-dioxo-2,5-dihydro-1H-pyrrol-1-yl)hexanoate (93-7, 20.14 mg, 0.065 mmol)) were added sequentially. The resulting solution was stirred for another 1 h until LCMS showed that the reaction was completed. The completed reaction solution was purified by directly by Prep-HPLC (0.01% TFA) to yield a TFA salt of product PB093 ({4-[(2S)-5-(carbamoylamino)-2-[(2S)-2-[(2S)-2-[6-(2,5-dioxo-2,5-dihydro-1H-pyrrol-1-yl)hexanamido]-6-[(42S,43R,44R)-42,43,44,45-tetrahydroxy-40-[(2S,3R,4R)-2,3,4,5-tetrahydroxypentyl]-4,7,10,13,16,19,22,25,28,31,34,37-dodecaoxa-40-azapentatetracontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB093, 51.21 mg, 0.025 mmol, 42.12%)) as a white solid. ESI m/z: 677.5 (M/3+H)⁺, retention time 5.924 min (HPLC). ¹H NMR (400 MHz, DMSO) b 10.02 (s, 1H), 8.19 (brs, 1H), 8.12 (d, J=7.2 Hz, 1H), 8.06 (d, J=8.8 Hz, 1H), 7.96 (d, J=8.0 Hz, 1H), 7.82-7.76 (m, 2H), 7.66 (d, J=8.8 Hz, 1H), 7.61 (d, J=8.4 Hz, 2H), 7.36 (d, J=8.4 Hz, 2H), 7.32 (s, 1H), 7.00 (s, 2H), 6.53 (s, 1H), 6.00 (t, J=5.6 Hz, 1H), 5.46-5.33 (m, 6H), 5.33-5.23 (m, 3H), 5.09 (s, 2H), 4.89-4.59 (m, 6H), 4.41-4.35 (m, 1H), 4.29-4.18 (m, 2H), 4.03-3.94 (m, 2H), 3.79-3.77 (m, 2H), 3.59-3.56 (m, 8H), 3.54-3.37 (m, 52H), 3.28-3.22 (m, 2H), 3.15-2.89 (m, 6H), 2.38 (s, 3H), 2.29 (t, J=6.4 Hz, 2H), 2.24-2.08 (m, 4H), 2.01-1.82 (m, 3H), 1.73-1.56 (m, 3H), 1.53-1.43 (m, 6H), 1.43-1.36 (m, 3H), 1.33-1.16 (m, 4H), 0.94-0.74 (m, 9H). One proton of carboxyl group in TFA was revealed.

### Example 25: Preparation of a Drug-Linker Containing a PEG Unit and a Cleavable Linker Attached to Exatecan (PB094)

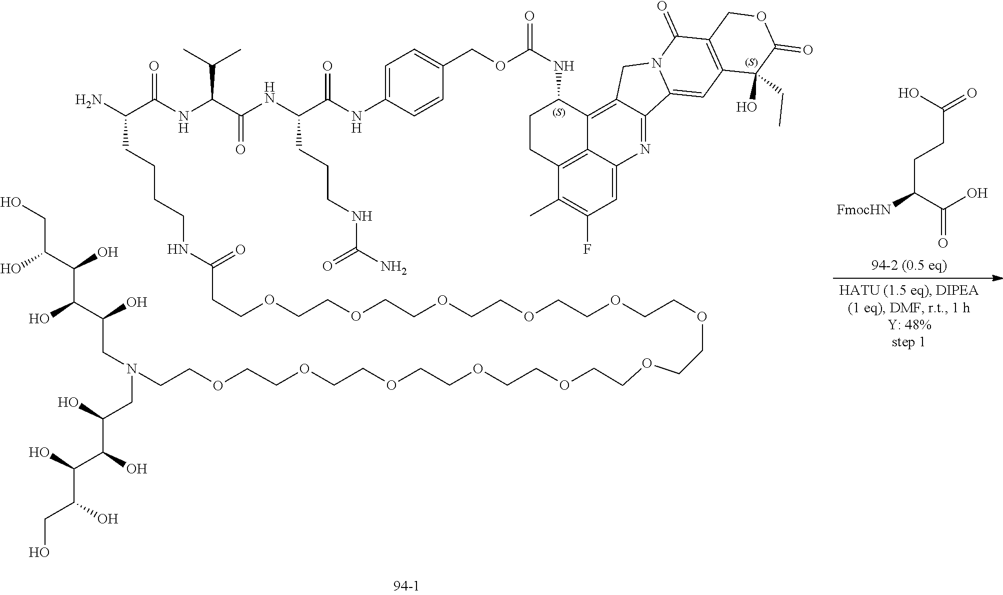

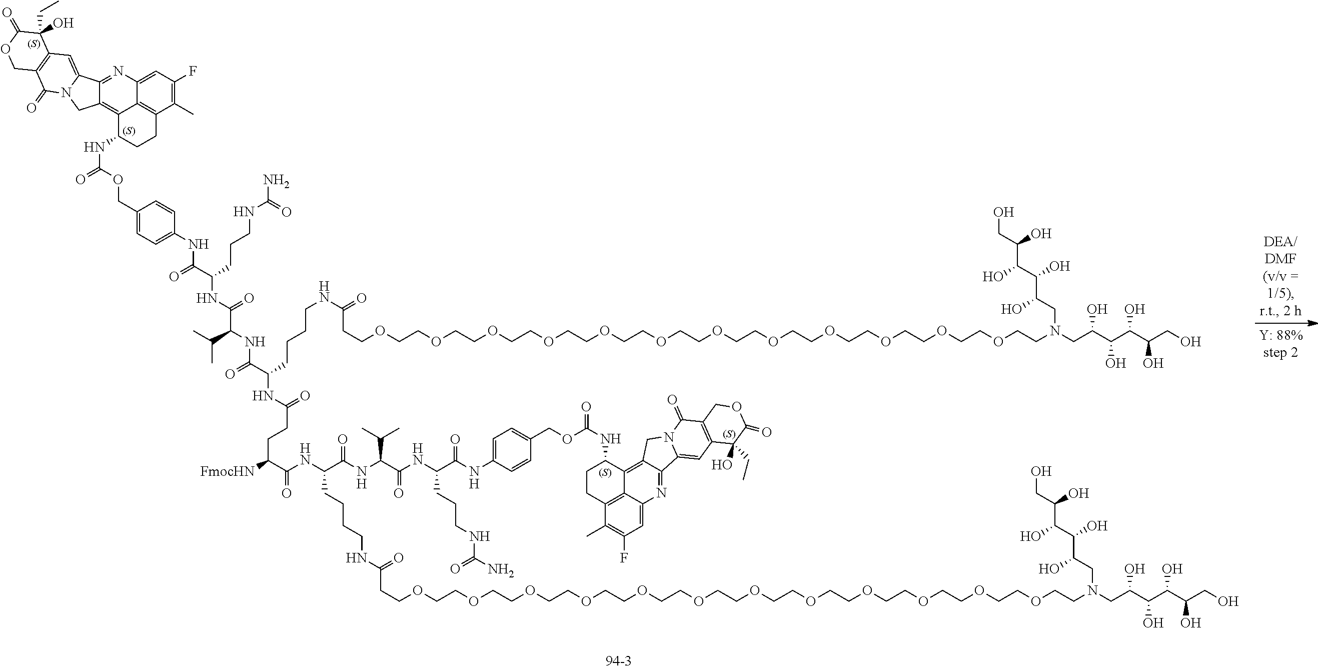

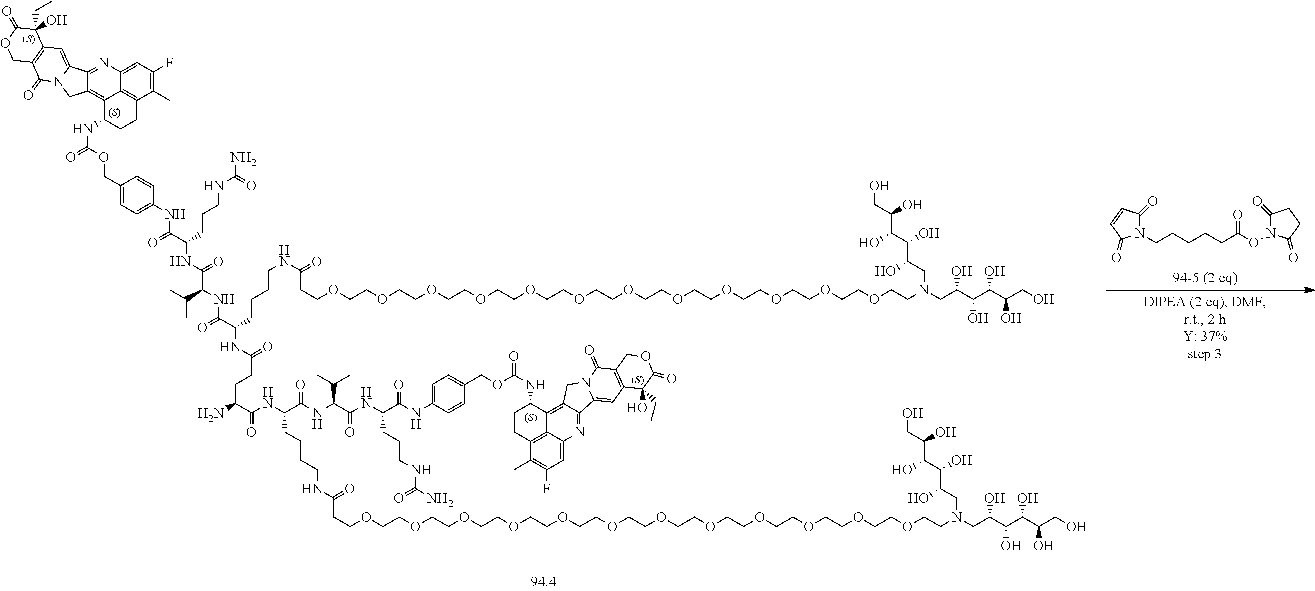

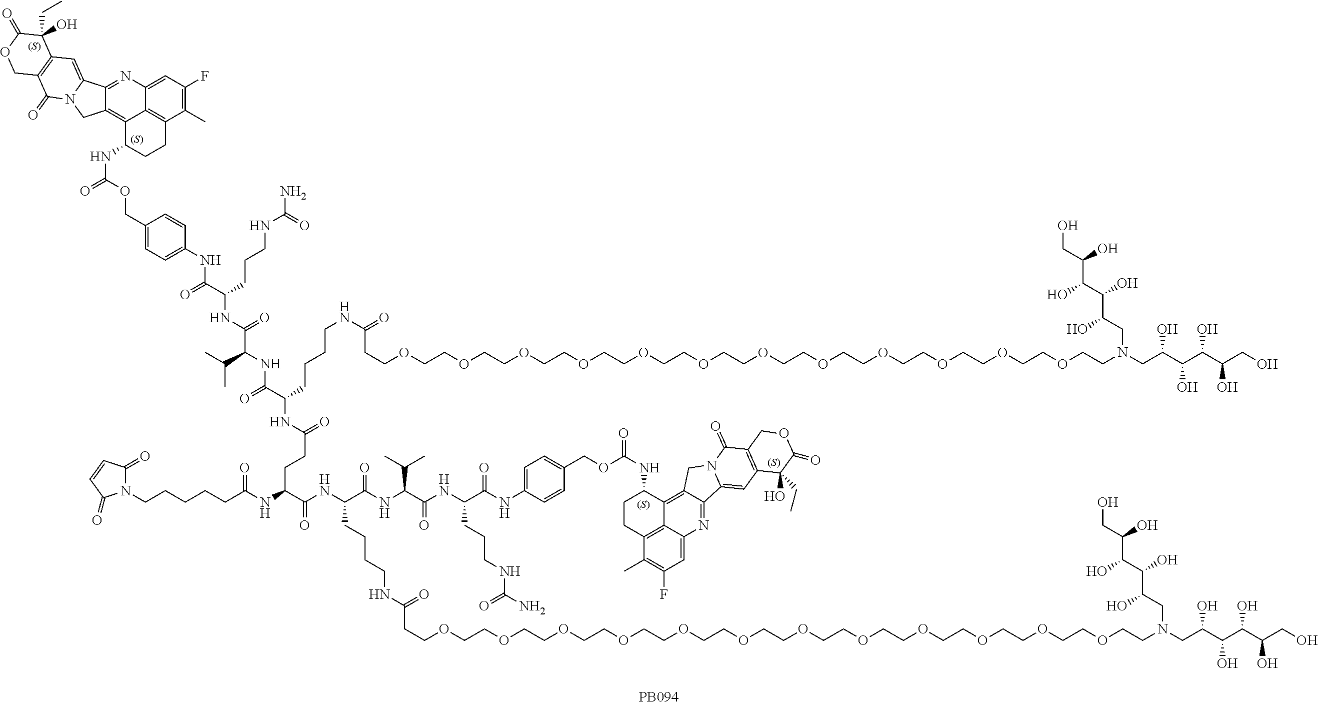

A Drug-Linker containing a PEG unit and a cleavable linker attached to exatecan (PB094) was prepared as follows:

### Step 1

To a solution of compound 94-2 ((2S)-2-({[(9H-fluoren-9-yl)methoxy]carbonyl}amino)pentanedioic acid (94-2, 19.39 mg, 0.052 mmol)) in DMF (5 mL) was added HATU (59.89 mg, 0.158 mmol) and DIPEA (13.57 mg, 0.105 mmol), followed by compound 94-1 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (94-1, 200 mg, 0.105 mmol)). The resulting mixture was stirred at room temperature for 1h to complete (monitored by LCMS). Then the resulting solution was purified by reverse phase flash chromatography (0.01% TFA) to afford product 94-3 ((9H-fluoren-9-yl)methyl N-[(1S)-1,3-bis({[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl})propyl]carbamate (94-3, 108 mg, 0.026 mmol, 49.84%)) as a white solid. ESI m/z: 826.4(M/5+H)⁺.

### Step 2

To a solution of compound 94-3 ((9H-fluoren-9-yl)methyl N-[(1S)-1,3-bis({[(1S)-1-{[(1 S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl})propyl]carbamate (94-3, 108 mg, 0.026 mmol)) in DMF (3 mL) was added diethyl amine (0.6 mL, 3.751 mmol). The mixture was stirred at room temperature for 2h. The resulting solution was adjusted to pH 6 with TFA and then purified by reverse phase flash chromatography (0.01% TFA) to afford TFA salt of product 94-4 ({4-[(2S)-2-[(2S)-2-[(2S)-2-[(2S)-2-amino-4-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}butanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (94-4, 90 mg, 0.023 mmol, 88.08%)) as a white solid. ESI m/z: 977.3(M/4+H)⁺.

### Step 3

To a solution of compound 94-4 ({4-[(2S)-2-[(2S)-2-[(2S)-2-[(2S)-2-amino-4-{[(1S)-1-{[(1 S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}butanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (94-4, 90 mg, 0.023 mmol)) in DMF (3 mL) was added DIPEA (5.96 mg, 0.046 mmol) and 2,5-dioxopyrrolidin-1-yl 6-(2,5-dioxo-2,5-dihydro-1H-pyrrol-1-yl)hexanoate (14.21 mg, 0.046 mmol). The mixture was stirred at room temperature for 2h. The resulting solution was adjusted to pH 6 and purified by Prep-HPLC (0.01% TFA) to afford the product PB094 ({4-[(2S)-5-(carbamoylamino)-2-[(2S)-2-[(2S)-2-[(2S)-4-{[(1S)-1-{[(1 S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-2-[6-(2,5-dioxo-2,5-dihydro-1 H-pyrrol-1-yl)hexanamido]butanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB094, 35 mg, 0.009 mmol, 37.06%)) as a white solid. ESI m/z: 820.7(M+H)⁺, 1025.5(M+H)⁺, retention time 5.452 min (HPLC). ¹H NMR (400 MHz, DMSO-d6) δ 10.04 (s, 2H), 8.19-8.15 (m, 4H), 8.14-8.12 (m, 4H), 8.07-8.05 (m, 1H), 7.97 (d, J=8.0 Hz, 1H), 7.89-7.82 (m, 3H), 7.76 (d, J=10.8 Hz, 2H), 7.62-7.60 (m, 4H), 7.37-7.35 (m, 4H), 7.31 (s, 2H), 7.00 (s, 2H), 6.53 (s, 2H), 6.00 (t, J=6.6 Hz, 2H), 5.51-5.44 (m, 12H), 5.34-5.21 (m, 6H), 5.08 (s, 4H), 4.88-4.70 (m, 4H), 4.55-4.43 (m, 8H), 4.38-4.33 (m, 3H), 4.29-4.25 (m, 2H), 4.20-4.14 (m, 3H), 4.01-3.99 (m, 4H), 3.79-3.78 (m, 4H), 3.69-3.66 (m, 4H), 3.62-3.56 (m, 18H), 3.53-3.48 (m, 88H), 3.46-3.42 (m, 10H), 3.27-3.12 (m, 7H), 3.12-2.90 (m, 12H), 2.37 (s, 6H), 2.31-2.27 (m, 4H), 2.21-2.01 (m, 8H), 1.95-1.79 (m, 8H), 1.73-1.55 (m, 8H), 1.49-1.40 (m, 8H), 1.40-1.31 (m, 6H), 1.31-1.14 (m, 6H), 0.89-0.82 (m, 18H) ppm. Two protons of carboxyl group in TFA were revealed.

### Example 26: Preparation of a Drug-Linker Containing a PEG Unit and a Cleavable Linker Attached to MMAE (PB095)

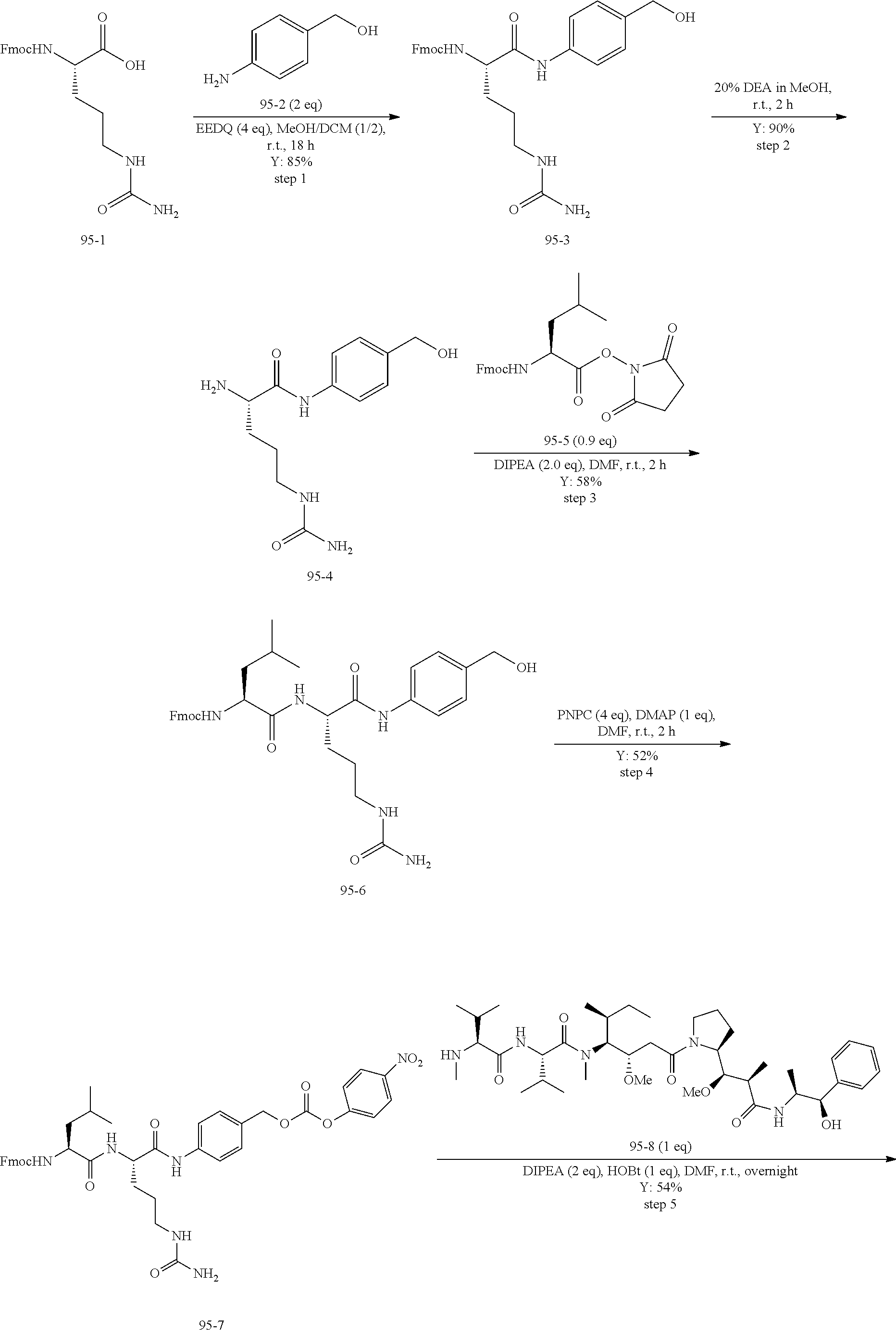

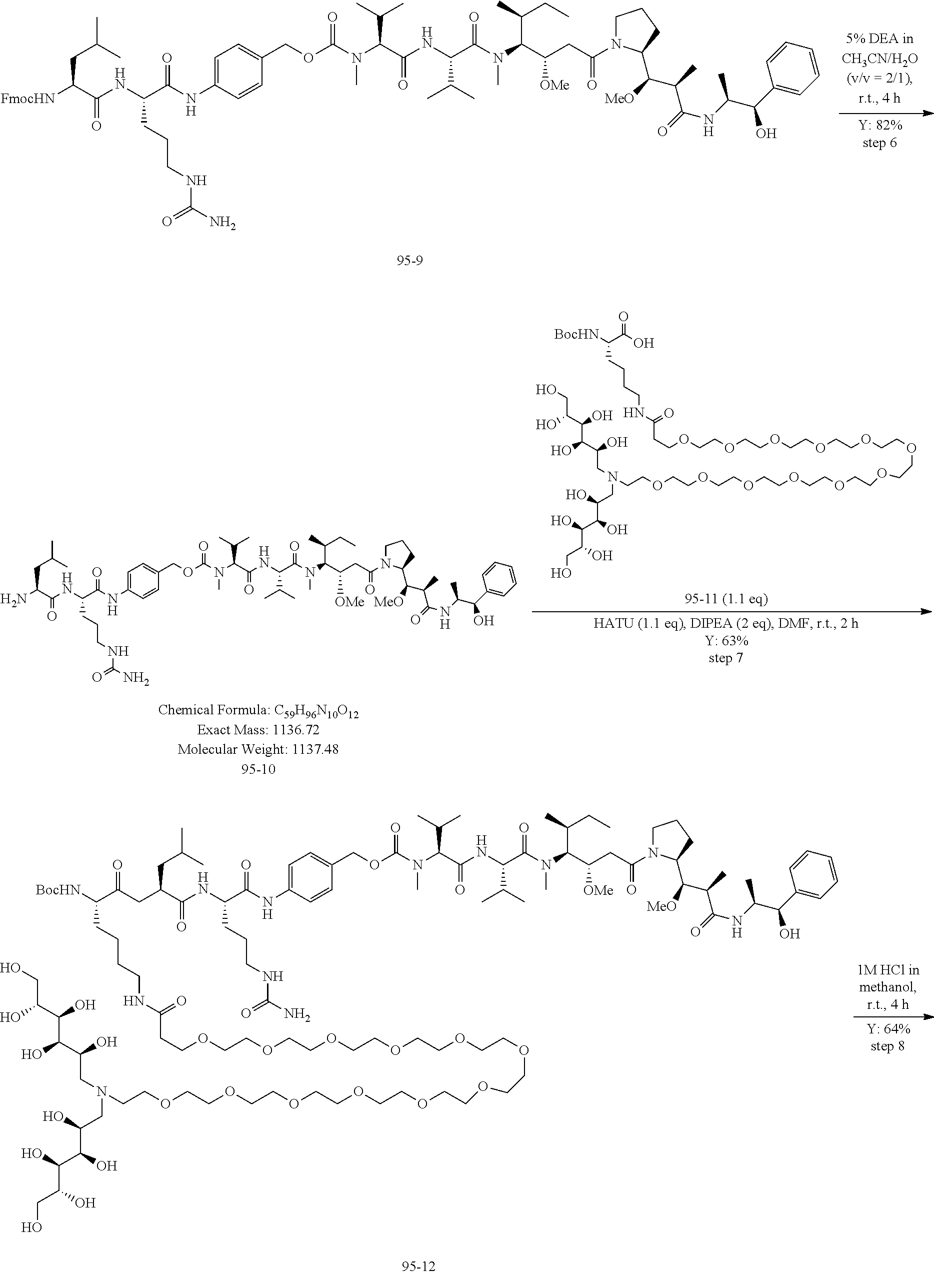

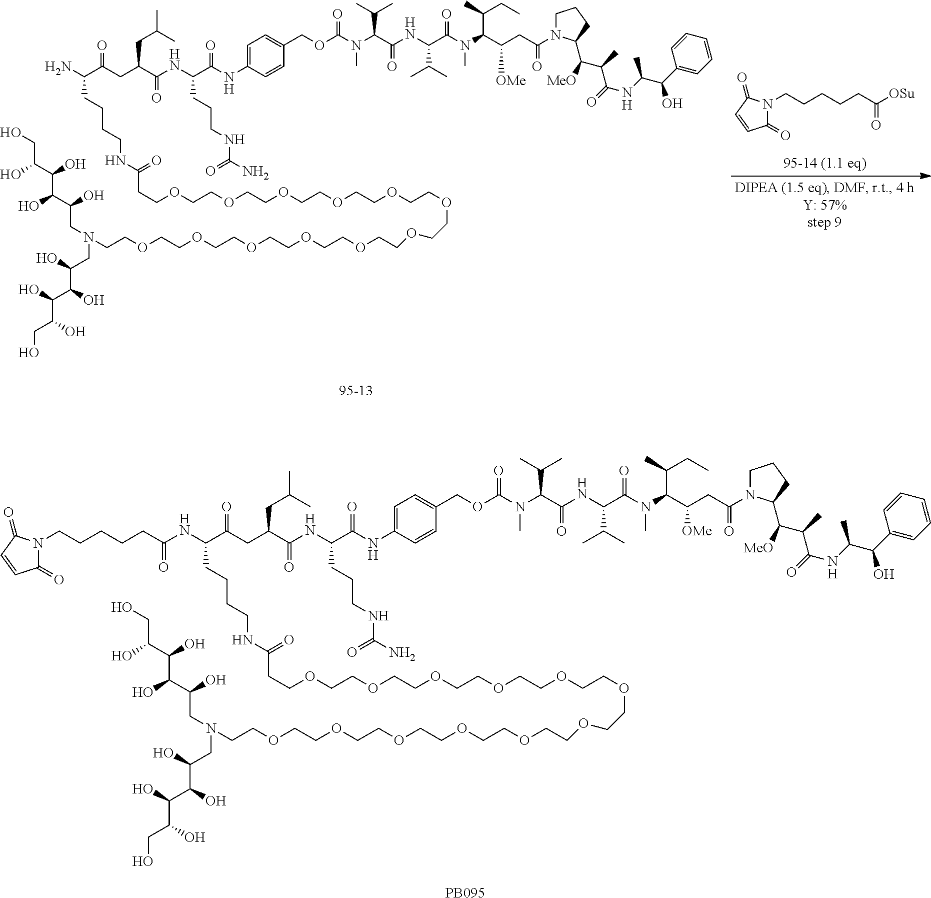

A Drug-Linker containing a PEG unit and a cleavable linker attached to MMAE (PB095) was prepared as follows:

### Step 1

A solution of compound 95-1 (2S)-5-(carbamoylamino)-2-({[(9H-fluoren-9-yl)methoxy]carbonyl}amino)pentanoic acid (95-1, 6.0 g, 15.097 mmol), compound 95-2 ((4-aminophenyl)methanol (95-2, 3.72 g, 30.195 mmol)) and EEDQ (14.93 g, 60.390 mmol) in MeOH (25 mL) and DCM (50 mL) was stirred at room temperature for 18 h and LCMS showed that the reaction was completed. The reaction solution was concentrated to dryness and then purified by reverse phase flash chromatography (0.01% TFA) to yield the desired fractions, which were freeze-dried to yield compound 95-3 ((9H-fluoren-9-yl)methyl N-[(1S)-4-(carbamoylamino)-1-{[4-(hydroxymethyl)phenyl]carbamoyl}butyl]carbamate (95-3, 6.45 g, 12.834 mmol, 85.01%)) as a white solid. ESI m/z: 503.3(M+H)⁺.

### Step 2

To a solution of compound 95-3 ((9H-fluoren-9-yl)methyl N-[(1S)-4-(carbamoylamino)-1-{[4-(hydroxymethyl)phenyl]carbamoyl}butyl]carbamate (95-3, 6.45 g, 12.834 mmol)) in MeOH (20 mL) was added diethyl amine (5 mL, 31.260 mmol). The mixture was stirred at room temperature for 2 h to achieve complete deprotection. The solution was concentrated and purified by reverse phase flash chromatography (0.01% TFA) to yield product 95-4 ((2S)-2-amino-5-(carbamoylamino)-N-[4-(hydroxymethyl)phenyl]pentanamide (95-4, 3.25 g, 11.594 mmol, 90.34%)) as a pale yellow solid. ESI m/z: 281.3(M+H)⁺.

### Step 3

A solution of compound 95-4 ((2S)-2-amino-5-(carbamoylamino)-N-[4-(hydroxymethyl)phenyl]pentanamide (95-4, 3.76 g, 13.413 mmol)), compound 95-5 (2,5-dioxopyrrolidin-1-yl (2S)-2-({[(9H-fluoren-9-yl)methoxy]carbonyl}amino)-4-methylpentanoate (95-5, 6.65 g, 14.755 mmol)) and DIPEA (3.47 g, 26.826 mmol) in DMF (10 mL) was stirred at room temperature for 2 h to completion. The reaction mixture was purified by reverse phase flash chromatography (0.01% TFA) to yield the desired fractions, which were freeze-dried to yield compound 95-6 ((9H-fluoren-9-yl)methyl N-[(1S)-1-{[(1S)-4-(carbamoylamino)-1-{[4-(hydroxymethyl)phenyl]carbamoyl}butyl]carbamoyl}-3-methylbutyl]carbamate (95-6, 4.8 g, 7.796 mmol, 58.12%)) as a white solid. ESI m/z: 616.3 (M+H)⁺. ¹H NMR (400 MHz, DMSO) b 9.97 (s, 1H), 8.05 (d, J=8.0 Hz, 1H), 7.89 (d, J=7.2 Hz, 2H), 7.74-7.70 (m, 2H), 7.55-7.51 (m, 3H), 7.44-7.39 (m, 2H), 7.34-7.30 (m, 2H), 7.23 (d, J=8.4 Hz, 2H), 5.97 (t, J=5.6 Hz, 1H), 5.41 (s, 2H), 5.10 (t, J=5.6 Hz, 1H), 4.43-4.39 (m, 3H), 4.32-4.20 (m, 3H), 4.15-4.05 (m, 1H), 3.07-2.89 (m, 2H), 1.74-1.55 (m, 3H), 1.51-1.32 (m, 4H), 0.90-0.83 (m, 6H) ppm.

### Step 4

A solution of compound 95-6 ((9H-fluoren-9-yl)methyl N-[(1S)-1-{[(1S)-4-(carbamoylamino)-1-{[4-(hydroxymethyl)phenyl]carbamoyl}butyl]carbamoyl}-3-methylbutyl]carbamate (95-6, 2.0 g, 3.248 mmol)), bis(4-nitrophenyl) carbonate (3.95 g, 12.993 mmol) and DMAP (0.40 g, 3.248 mmol) in DMF (5 mL) was stirred at room temperature for 2 h. Then the reaction mixture was quenched with drops of water and purified by reverse phase flash chromatography (neutral eluent) to yield the desired fractions, which were freeze-dried to yield compound 95-7 ({4-[(2S)-5-(carbamoylamino)-2-[(2S)-2-({[(9H-fluoren-9-yl)methoxy]carbonyl}amino)-4-methylpentanamido]pentanamido]phenyl}methyl 4-nitrophenyl carbonate (95-7, 1.33 g, 1.703 mmol, 52.44%)) as a pale yellow solid. ESI m/z: 781.3 (M+H)⁺.

### Step 5

A solution of compound 95-7 ({4-[(2S)-5-(carbamoylamino)-2-[(2S)-2-({[(9H-fluoren-9-yl)methoxy]carbonyl}amino)-4-methylpentanamido]pentanamido]phenyl}methyl 4-nitrophenyl carbonate (95-7, 340 mg, 0.435 mmol)), compound 95-8 ((2S)—N-[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]-3-methyl-2-(methylamino)butanamide (95-8, 312.63 mg, 0.435 mmol)) and HOBt (58.84 mg, 0.435 mmol) in anhydrous DMF (4 mL) was stirred at room temperature for 5 min, then DIPEA (112.34 mg, 0.871 mmol) was added. The resulting yellow solution was stirred for overnight until LCMS indicated both starting materials were substantially consumed. The resulting solution was purified directly by reverse phase flash chromatography (0.01% TFA) to yield compound 95-9 ((9H-fluoren-9-yl)methyl N-[(1S)-1-{[(1 S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-3-methylbutyl]carbamate (95-9, 320 mg, 0.235 mmol, 54.10%)) as a white solid. ESI m/z: 680.6 (M/2+H)⁺; 718.7 (fragment piece of MMAE); 598.4 (fragment piece of Linker Fmoc-Leu-Cit-PAB).

### Step 6

A solution of compound 95-9 ((9H-fluoren-9-yl)methyl N-[(1S)-1-{[(1S)-4-(carbamoylamino)-1-({4-[({[(1S)-1-{[(1 S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-3-methylbutyl]carbamate (95-9, 320 mg, 0.235 mmol)) in CH₃CN (10 mL) and water (5 mL) was stirred at room temperature and diethyl amine (0.8 mL, 7.766 mmol) was added. The resulting solution was stirred for 4 h to complete. Diethyl amine was evaporated off under vacuo and the residue was purified by reverse phase flash chromatography (0.01% TFA) to yield a TFA salt of product 95-10 ({4-[(2S)-2-[(2S)-2-amino-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-10, 220 mg, 0.193 mmol, 82.18%)) as a white solid and the product was used for next step without any purification. ESI m/z: 569.5(M/2+H)⁺.

¹H NMR (400 MHz, DMSO-d6) δ 10.21 (s, 1H), 8.79 (d, J=7.6 Hz, 1H), 8.31-8.06 (m, 4H), 7.91 (d, J=8.8 Hz, 0.5H), 7.65 (d, J=8.4 Hz, 0.5H), 7.60-7.57 (m, 2H), 7.37-7.24 (m, 6H), 7.20-7.14 (m, 1H), 6.08 (s, 1H), 5.60-5.31 (m, 2H), 5.12-4.98 (m, 2H), 4.74-4.62 (m, 1H), 4.55-4.40 (m, 3H), 4.30-4.23 (m, 1H), 4.05-3.92 (m, 2H), 3.85-3.76 (m, 2H), 3.58-3.55 (m, 2H), 3.32-3.12 (m, 9H), 3.08-2.97 (m, 4H), 2.89-2.83 (m, 3H), 2.44-2.39 (m, 1H), 2.29-2.23 (m, 1H), 2.16-1.92 (m, 3H), 1.84-1.39 (m, 13H), 1.31-1.25 (m, 1H), 1.06-0.97 (m, 6H), 0.92-0.75 (m, 24H) ppm. One proton of carboxyl group in TFA is revealed.

### Step 7

A solution of compound 95-10 ({4-[(2S)-2-[(2S)-2-amino-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-10, 218 mg, 0.192 mmol)), compound 95-11 ((2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (95-11, 247.57 mg, 0.211 mmol)) and DIPEA (49.45 mg, 0.383 mmol) in anhydrous DMF (3 mL) was stirred at room temperature for 5 min to ensure the starting acid dissolve completely, and then a solution of HATU (80.16 mg, 0.211 mmol) in anhydrous DMF (1 mL) was added slowly. The resulting solution was stirred for another 2 h until LCMS indicated a complete reaction. Then the reaction solution was purified directly by reverse phase flash chromatography (0.01% TFA) to give compound 95-12 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-12, 280 mg, 0.122 mmol, 63.69%)) as a white solid. ESI m/z: 765.5 (M/3+H)⁺.

### Step 8

A solution of compound 95-12 ({4-[(2S)-2-[(2S)-2-[(2S)-2-{[(tert-butoxy)carbonyl]amino}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-12, 260 mg, 0.113 mmol)) in methanol was stirred at room temperature, and 3M HCl in MeOH (2 mL) was added slowly. The resulting light yellow solution was kept stirring for another 4 h until complete deprotection was achieved (monitored by LCMS). Then the solution was concentrated with a rotary evaporator to remove solvent, the residue was dissolved with water, and purified by reverse phase flash chromatography (0.01% TFA) to yield compound 95-13 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-13, 160 mg, 0.073 mmol, 64.35%)) as a white solid. ESI m/z: 732.2 (M/3+H)⁺, 718.6 (fragment MMAE), 478.2 ((Linker_{Boc-Lys(PEG12-sugar)-Leu-Cit-pab}-18)/3+H)⁺.

### Step 9

A solution of compound 95-13 ({4-[(2S)-2-[(2S)-2-[(2S)-2-amino-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (95-13, 80 mg, 0.036 mmol)) and DIPEA (7.06 mg, 0.055 mmol) in anhydrous DMF (1.5 mL) was stirred at room temperature for 5 min to dissolve all materials. Then a solution of 2,5-dioxopyrrolidin-1-yl 6-(2,5-dioxo-2,5-dihydro-1H-pyrrol-1-yl)hexanoate (95-14, 12.36 mg, 0.040 mmol) in anhydrous DMF (0.5 mL) was added dropwise by syringe over 5 min. After addition, the resulting solution was stirred for another 4h until LCMS indicated all starting amine was consumed. The resulting solution was then purified directly by Prep-HPLC (0.01% TFA) to yield the desired fractions, which were lyophilized by LabConc to yield a TFA salt of PB095 ({4-[(2S)-5-(carbamoylamino)-2-[(2S)-2-[(2S)-2-[6-(2,5-dioxo-2,5-dihydro-1 H-pyrrol-1-yl)hexanamido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-4-methylpentanamido]pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB095, 50 mg, 0.021 mmol, 57.44%)) as a white solid. ESI m/z: 542.5 ((linker fragment, (M-717-26-18)3+H)⁺; 796.6 (M/3+H)⁺. Retention time 6.250 min (HPLC). ¹H NMR (400 MHz, DMSO-d6) δ 10.00 (s, 1H), 8.34-8.07 (m, 1.5 H), 8.00 (d, J=7.6 Hz, 1H), 7.93-7.88 (m, 2.5 H), 7.82-7.79 (m, 1H), 7.65-7.57 (m, 2.5 H), 7.35-7.23 (m, 6H), 7.18-7.10 (m, 1.5H), 7.00 (s, 2H), 5.99-5.98 (m, 1H), 5.53-5.25 (m, 4H), 5.14-4.95 (m, 2H), 4.83-4.17 (m, 11H), 4.04-3.92 (m, 4H), 3.79-3.77 (m, 2H), 3.70-3.66 (m, 2H), 3.61-3.56 (m, 8H), 3.53-3.45 (m, 44H), 3.25-3.12 (m, 14H), 3.05-2.83 (m, 14H), 2.44-2.39 (m, 1H), 2.31-2.14 (m, 4H), 2.14-1.96 (m, 6H), 1.85-1.44 (m, 18H), 1.36-1.16 (m, 10H), 1.06-0.97 (m, 6H), 0.89-0.75 (m, 27H) ppm. One proton of carboxyl group in TFA was revealed.

### Example 27: Preparation of a Drug-Linker Containing a Peg Unit and a Cleavable Linker Attached to Mmae (Pb096)

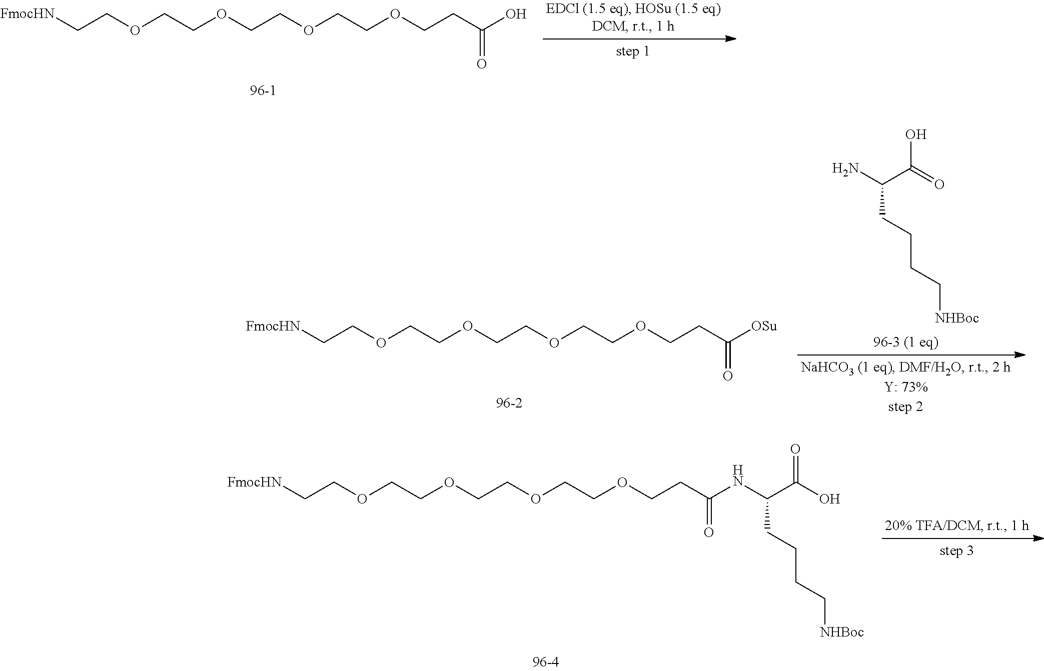

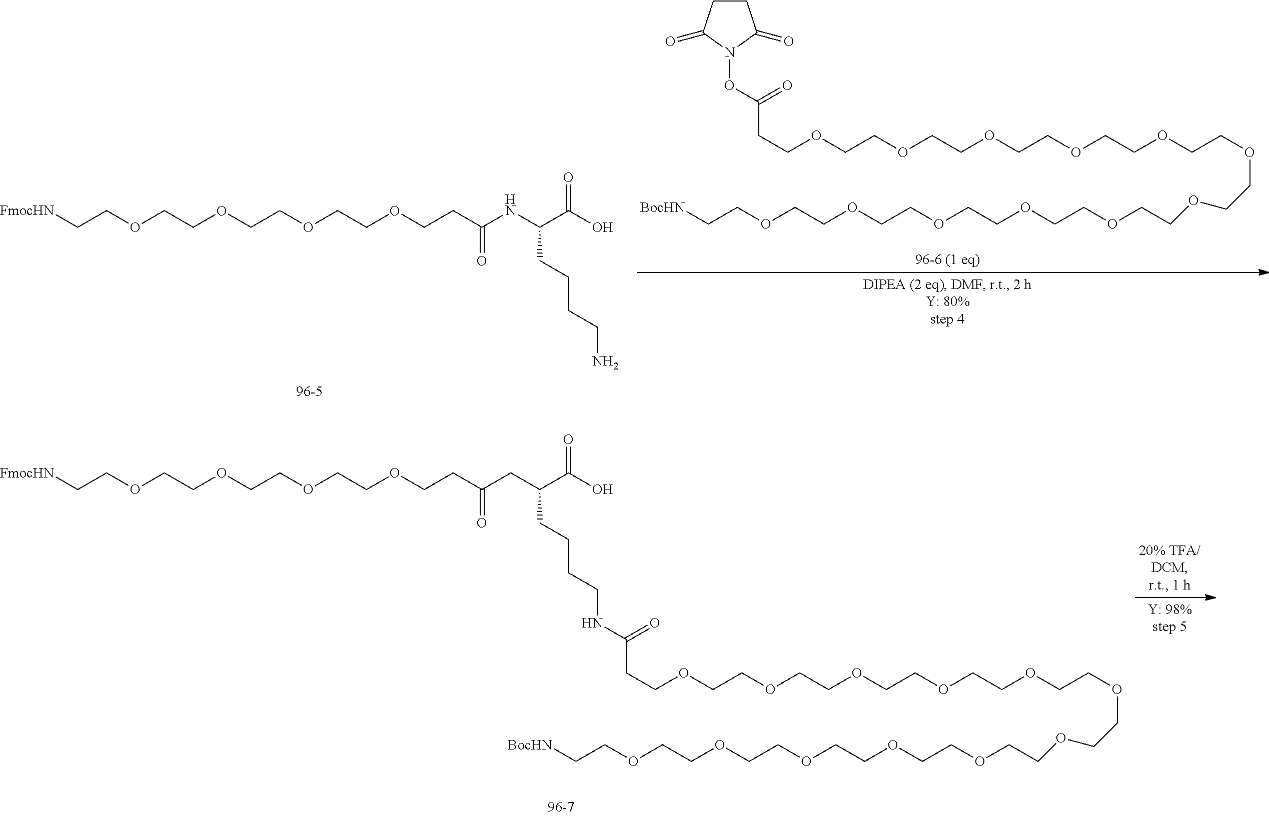

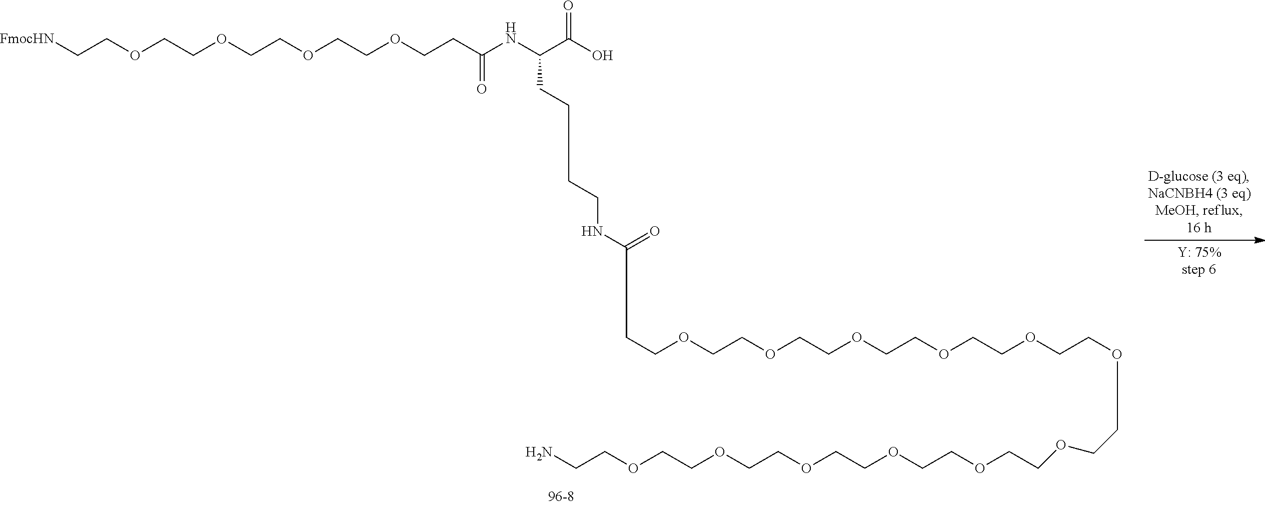

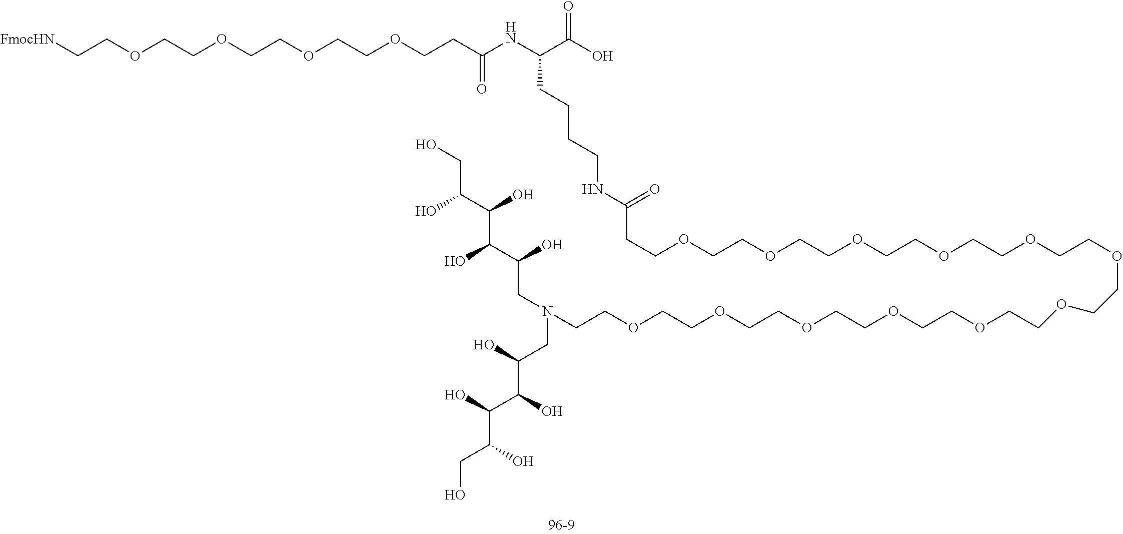

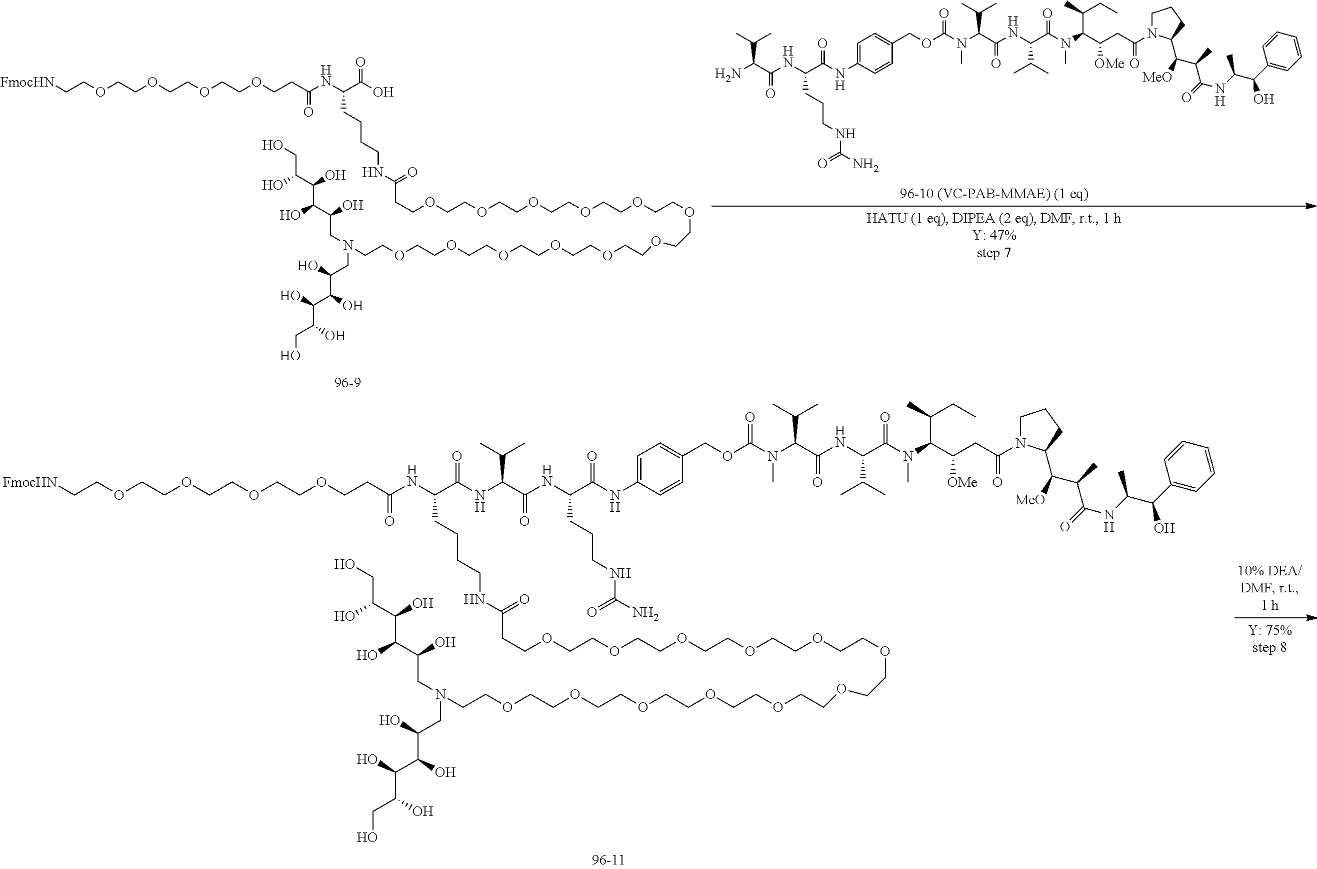

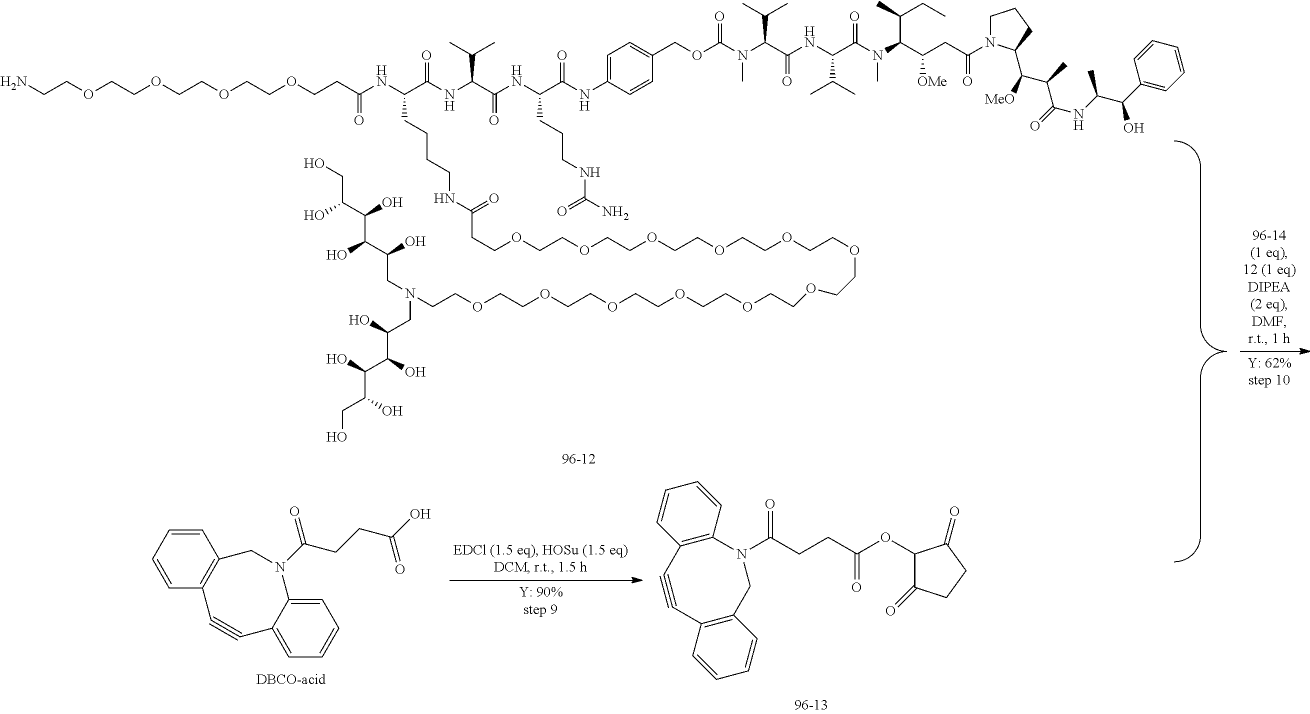

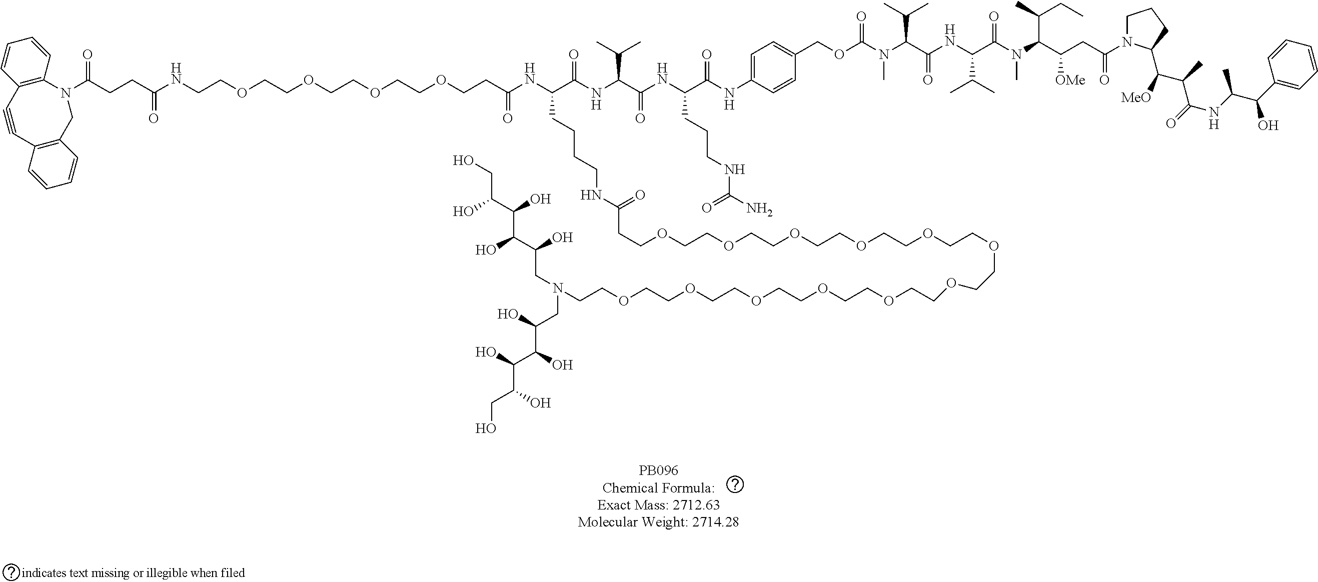

A Drug-Linker containing a PEG unit and a cleavable linker attached to MMAE (PB096) was prepared as follows:

### Step 1

A clear solution of compound 96-1 (1-({[(9H-fluoren-9-yl)methoxy]carbonyl}amino)-3,6,9,12-tetraoxapentadecan-15-oic acid (96-1, 5.00 g, 10.267 mmol)) and HOSu (1.77 g, 15.401 mmol) in dry DCM (40 mL) was stirred at room temperature and EDCl (2.95 g, 15.401 mmol) was added. The solution was kept stirring for 1h until complete conversion was achieved. Then the solution was diluted with more DCM (20 mL) and washed with water (50 mL), the organic layer was separated and the water layer was extracted with DCM (50 mL*2). The combined collected DCM phase was dried over sodium sulfate and filtered, then concentrated under reduced pressure to yield crude material as a colorless oil compound 96-2, which was used as such in the next step. ESI m/z=585.3 (M+H)⁺.

### Step 2

A suspension of compound 96-3 (N6-(tert-butoxycarbonyl)-L-lysine (96-3, 2.233 mL, 10.267 mmol)) in DMF (12 mL) was stirred at room temperature, then a solution of sodium bicarbonate (0.86 g, 10.267 mmol) in water (3 mL) was added. The suspension was stirred for 20 min until most of starting acid was dissolved in the solvent. Then compound 96-2 (2,5-dioxopyrrolidin-1-yl 1-(9H-fluoren-9-yl)-3-oxo-2,7,10,13,16-pentaoxa-4-azanonadecan-19-oate (96-2, 6.00 g, 10.267 mmol)) was added. The resulting solution was stirred at room temperature for 2h. The completed reaction solution was purified directly by reverse phase column chromatography (0.01% TFA) to yield compound 96-4 (N2-(1-(9H-fluoren-9-yl)-3-oxo-2,7,10,13,16-pentaoxa-4-azanonadecan-19-oyl)-N6-(tert-butoxycarbonyl)-L-lysine (96-4, 5.40 g, 7.552 mmol, 73.56%)) as a white solid. ESI m/z: 716.5 (M+H)⁺, 738.4 (M+Na)⁺. ¹H NMR (400 MHz, 400 MHz) b 7.89 (d, J=7.6 Hz, 2H), 7.86-7.77 (m, 1H), 7.69 (d, J=7.2 Hz, 2H), 7.44-7.38 (m, 2H), 7.37-7.31 (m, 2H), 6.75 (t, J=5.6 Hz, 1H), 4.30-4.19 (m, 2H), 4.04-3.99 (m, 1H), 3.59-3.54 (m, 2H), 3.50-3.47 (m, 12H), 3.43-3.39 (m, 2H), 3.16-3.11 (m, 2H), 2.88-2.83 (m, 2H), 2.40-2.32 (m, 2H), 1.67-1.63 (m, 1H), 1.55-1.48 (m, 1H), 1.36 (s, 9H), 1.33-1.29 (m, 2H), 1.25-1.20 (m, 2H), 0.99 (d, J=6.4 Hz, 2H) ppm. Carboxyl group is not revealed.

### Step 3

A solution of compound 96-4 ((2S)-6-{[(tert-butoxy)carbonyl]amino}-2-[1-({[(9H-fluoren-9-yl)methoxy]carbonyl}amino)-3,6,9,12-tetraoxapentadecan-15-amido]hexanoic acid (96-4, 5.4 g, 7.544 mmol)) in DCM (16 mL) was stirred at room temperature and then TFA (4 mL, 53.850 mmol) was added. The resulting yellow solution was stirred for another 1h. Then TFA and solvent were evaporated, the residue was dissolved in DCM again and concentrated to dryness. The process was repeated three times, and a crude TFA salt of product 96-5 ((1-(9H-fluoren-9-yl)-3-oxo-2,7,10,13,16-pentaoxa-4-azanonadecan-19-oyl)-L-lysine (96-5)) was obainted as a white solid. ESI m/z: 616.4(M+H)⁺.

### Step 4

A solution of 1-{[(tert-butoxy)carbonyl]amino}-3,6,9,12,15,18,21,24,27,30,33,36-dodecaoxanonatriacontan-39-oic acid (1.58 g, 2.201 mmol) and HOSu (0.53 g, 4.597 mmol) in anhydrous DCM (20 mL) was stirred at room temperature for 5 min, then EDCl (0.63 g, 3.302 mmol) was added. The resulting solution was stirred for another 1h, then was diluted with more DCM (20 mL) and washed with water (20 mL). The organic layer was collected and the water layer was extracted with DCM (40 mL*2). The combined DCM layer was dried over sodium sulfate, filtered and concentrated the filtrate to give crude product 96-6 (2,5-dioxopyrrolidin-1-yl 2,2-dimethyl-4-oxo-3,8,11,14,17,20,23,26,29,32,35,38,41-tridecaoxa-5-azatetratetracontan-44-oate (96-6, 2.5 g, 3.069 mmol, 100.15%)) as colorless oil. ESI m/z: 715.5 (M-100+H)⁺, 837.5(M+Na)⁺.

A solution of compound 96-5 ((1-(9H-fluoren-9-yl)-3-oxo-2,7,10,13,16-pentaoxa-4-azanonadecan-19-oyl)-L-lysine (96-5, 1.55 g, 2.514 mmol)) and compound 96-6 (2,5-dioxopyrrolidin-1-yl 2,2-dimethyl-4-oxo-3,8,11,14,17,20,23,26,29,32,35,38,41-tridecaoxa-5-azatetratetracontan-44-oate (96-6, 1.8 g, 2.211 mmol)) in anhydrous DMF (22 mL) was stirred at room temperature for 5 min, then DIPEA (0.57 g, 4.422 mmol) was added slowly by syringe. The resulting solution was stirred for 2 h until LCMS indicated all starting amine was consumed. The resulting solution was concentrated under reduced pressure and the residue was purified directly by Prep-HPLC (0.01% TFA) to yield compound 96-7 ((2S)-6-(1-{[(tert-butoxy)carbonyl]amino}-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-7, 2.3 g, 1.748 mmol, 79.04%)) as colorless oil. ESI m/z: 608.5 ((M-100)/2+H)⁺.

### Step 5

A solution of compound 96-7 ((2S)-6-(1-{[(tert-butoxy)carbonyl]amino}-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-7, 2.3 g, 1.748 mmol)) in DCM (16 mL) was stirred at r.t., then TFA (4 mL, 53.850 mmol) was added. The resulting solution was stirred for 1h to achieve complete deprotection. Then TFA and DCM were evaporated off, the residue was purified by reverse phase flash chromatography (0.01% TFA) to give the expected fractions, which were lyophilized to yield product 96-8 ((2S)-6-(1-amino-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-8, 2.1 g, 1.728 mmol, 99.06%)) as color less oil. ESI m/z: 608.5 (M+H)⁺. ¹H NMR (400 MHz, DMSO-d6) δ 7.89 (d, J=7.6 Hz, 2H), 7.86-7.80 (m, 2H), 7.71-7.61 (m, 1H), 7.44-7.40 (m, 2H), 7.37-7.33 (m, 2H), 6.29 (s, 1H), 4.30-4.21 (m, 1H), 4.06-3.95 (m, 2H), 3.68-3.55 (m, 12H), 3.55-3.47 (m, 48H), 3.42-3.38 (m, 2H), 3.38-3.21 (m, 4H), 3.16-3.05 (m, 2H), 3.00-2.94 (m, 4H), 2.43-2.37 (m, 2H), 2.33-2.27 (m, 2H), 1.70-1.57 (m, 1H), 1.58-1.44 (m, 1H), 1.38-1.18 (m, 4H) ppm. Two protons in amino group and two protons in carboxyl group are not revealed.

### Step 6

A suspension of compound 96-8 ((2S)-6-(1-amino-3,6,9,12,15,18,21,24,27,30,33,36-dodecaoxanonatriacontan-39-amido)-2-[1-({[(9H-fluoren-9-yl)methoxy]carbonyl}amino)-3,6,9,12-tetraoxapentadecan-15-amido]hexanoic acid (96-8, 2.0 g, 1.645 mmol)) and D-glucose (1.78 g, 9.873 mmol) in methanol (32 mL) was heated to 50° C. under nitrogen atmosphere and then sodium cyanoborohydride (0.62 g, 9.873 mmol) was added. The resulting mixture was heated overnight (16 h) until LCMS indicated complete reaction. The solution was then concentrated to dryness, and the residue was dissolved in water and purified by reverse phase flash chromatography (0.01% TFA) to give the desired fractions, which were freeze-dried over LabConco to yield compound 96-9 ((2S)-2-[1-({[(9H-fluoren-9-yl)methoxy]carbonyl}amino)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (96-9, 2.0 g, 1.296 mmol, 78.74%)) as colorless oil. ESI m/z: 515.5 (M/3+H)⁺, 772.6 (M/2+H)⁺. ¹H NMR (400 MHz, DMSO-d6) δ 8.02 (d, J=7.2 Hz, 1H), 7.89 (d, J=7.2 Hz, 2H), 7.83 (t, J=5.6 Hz, 1H), 7.69 (d, J=7.2 Hz, 2H), 7.44-7.39 (m, 2H), 7.35-7.31 (m, 3H), 4.76-4.37 (m, 4H), 4.30-4.28 (m, 2H), 4.23-4.19 (m, 1H), 4.15-4.09 (m, 1H), 4.03-3.91 (m, 1H), 3.75-3.64 (m, 2H), 3.59-3.56 (m, 6H), 3.56-3.46 (m, 56H), 3.41-3.17 (m, 18H), 3.17-3.12 (m, 2H), 3.03-2.97 (m, 2H), 2.97-2.54 (m, 5H), 2.41-2.34 (m, 2H), 2.29 (t, J=6.4 Hz, 2H), 1.71-1.65 (m, 1H), 1.60-1.51 (m, 1H), 1.38-1.31 (m, 2H), 1.31-1.24 (m, 2H) ppm. One proton in carboxyl group is not revealed.

### Step 7

A solution of compound 96-9 ((2S)-2-[1-({[(9H-fluoren-9-yl)methoxy]carbonyl}amino)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (96-9, 125 mg, 0.081 mmol)), compound 96-10 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (96-10, 90.97 mg, 0.081 mmol)) and DIPEA (20.89 mg, 0.162 mmol) in anhydrous DMF (1.5 mL) was stirred at room temperature for 5 min, then a solution of HATU (30.79 mg, 0.081 mmol) in anhydrous DMF was added dropwise by syringe over 5 min. After addition, the resulting solution was stirred for another 1 h until all starting amine was consumed (monitored by LCMS). Then the reaction solution was purified directly by reverse phase flash chromatography (0.01% TFA) to yield compound 96-11 ((9H-fluoren-9-yl)methyl N-(14-{[(1 S)-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (96-11, 100 mg, 0.038 mmol, 46.62%)) as a white solid, mixed with starting acid. ESI m/z: 883.9 (M/3+H)⁺, 630.0 (linker fragment, (M-717-26-18)/3+H)⁺, purity 60%-66%. Impurity acid ESI m/z: 515.5 (M/3+H)⁺,772.6 (M/2+H)⁺, content 35%-29%.

### Step 8

A solution of compound 96-11 ((9H-fluoren-9-yl)methyl N-(14-{[(1S)-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (96-11, 100 mg, 0.038 mmol)) in DMF (1.8 mL) was stirred at room temperature and diethyl amine (0.2 mL, 1.941 mmol) was added. The resulting solution was stirred for 1 h to completion. Volatiles were evaporated off under vacuo, and the residue in DMF was purified by reverse phase flash chromatography (0.01% TFA) to give the expected fractions, which were lyophilized by LabConco to yield product 96-12 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (96-12, 75 mg, 0.031 mmol, 81.32%)) as a white solid. ESI m/z: 809.9 (M/3+H)⁺, 718.6 (fragment, MMAE), 555.9 (linker fragment, (M-717-26-18)/3+H)⁺.

### Step 9

A solution of 4-{2-azatricyclo[10.4.0.0\hat{ }{4,9}]hexadeca-1(16),4(9),5,7,12,14-hexaen-10-yn-2-yl}-4-oxobutanoic acid (DBCO-acid, 710 mg, 2.328 mmol) and HOSu (401.56 mg, 3.492 mmol) in anhydrous DCM (23 mL) was stirred at room temperature for 5 min, then EDCl (669.38 mg, 3.492 mmol) was added. The resulting solution was stirred for another 1.5h. Then the resulting solution was washed with water (10 mL), the DCM layer was separated and the water layer was extracted with more DCM (20 mL*2). The combined DCM phase was concentrated to dryness and the residue was purified by reverse phase flash chromatography (neutral eluent) to yield compound 96-13 (2,5-dioxopyrrolidin-1-yl 4-{2-azatricyclo[10.4.0.0{4,9}]hexadeca-1(12),4(9),5,7,13,15-hexaen-10-yn-2-yl}-4-oxobutanoate (96-13, 850 mg, 2.114 mmol, 90.83%)) as a white solid. ESI m/z: 403.2 (M+H)⁺, 425.2 (M+Na)⁺.

### Step 10

A solution of compound 96-12 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (96-12, 65 mg, 0.027 mmol)) and DIPEA (6.91 mg, 0.054 mmol) in anhydrous DMF (2 mL) was stirred at room temperature for 5 min, then a solution of DBCO NHS ester (compound 96-13, 10.78 mg, 0.027 mmol) in anhydrous DMF (2 mL) was added dropwise by syringe. The resulting solution was stirred for another 1 h until LCMS indicated all starting amine was consumed. The resulting solution was then purified directly by Prep-HPLC (10 mM ammonium bicarbonate) to yield PB096 ({4-[(2S)-2-[(2S)-2-[(2S)-2-[1-(4-{2-azatricyclo[10.4.0.0{4,9}]hexadeca-1(12),4(9),5,7,13,15-hexaen-10-yn-2-yl}-4-oxobutanamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB096, 45 mg, 0.017 mmol, 61.90%)) as a white solid. ESI m/z: 905.3 (M/3+H)⁺, 679.2 (M/4+H)⁺, retention time 5.577 min (HPLC). ¹H NMR (400 MHz, DMSO-d6) δ 10.05 (s, 1H), 8.37-8.10 (m, 2H), 8.05-8.03 (m, 1H), 7.93-7.90 (m, 1H), 7.83-7.77 (m, 2H), 7.77-7.67 (m, 2H), 7.63-7.57 (m, 3H), 7.51-7.45 (m, 3H), 7.38-7.26 (m, 9H), 7.20-7.14 (m, 1H), 6.00-5.97 (m, 1H), 5.43-5.36 (m, 3H), 5.11-4.94 (m, 3H), 4.76-4.17 (m, 16H), 4.04-3.92 (m, 2H), 3.79-3.57 (m, 12H), 3.50-3.41 (m, 56H), 3.36-3.17 (m, 19H), 3.12-2.85 (m, 15H), 2.60-2.56 (m, 2H), 2.43-2.36 (m, 3H), 2.30-2.22 (m, 4H), 2.15-1.96 (m, 6H), 1.78-1.21 (m, 20H), 1.05-0.97 (m, 6H), 0.89-0.77 (m, 24H) ppm.

### Example 28: Preparation of a Drug-Linker Containing a PEG Unit and a Cleavable Linker Attached to Exatecan (PB097)

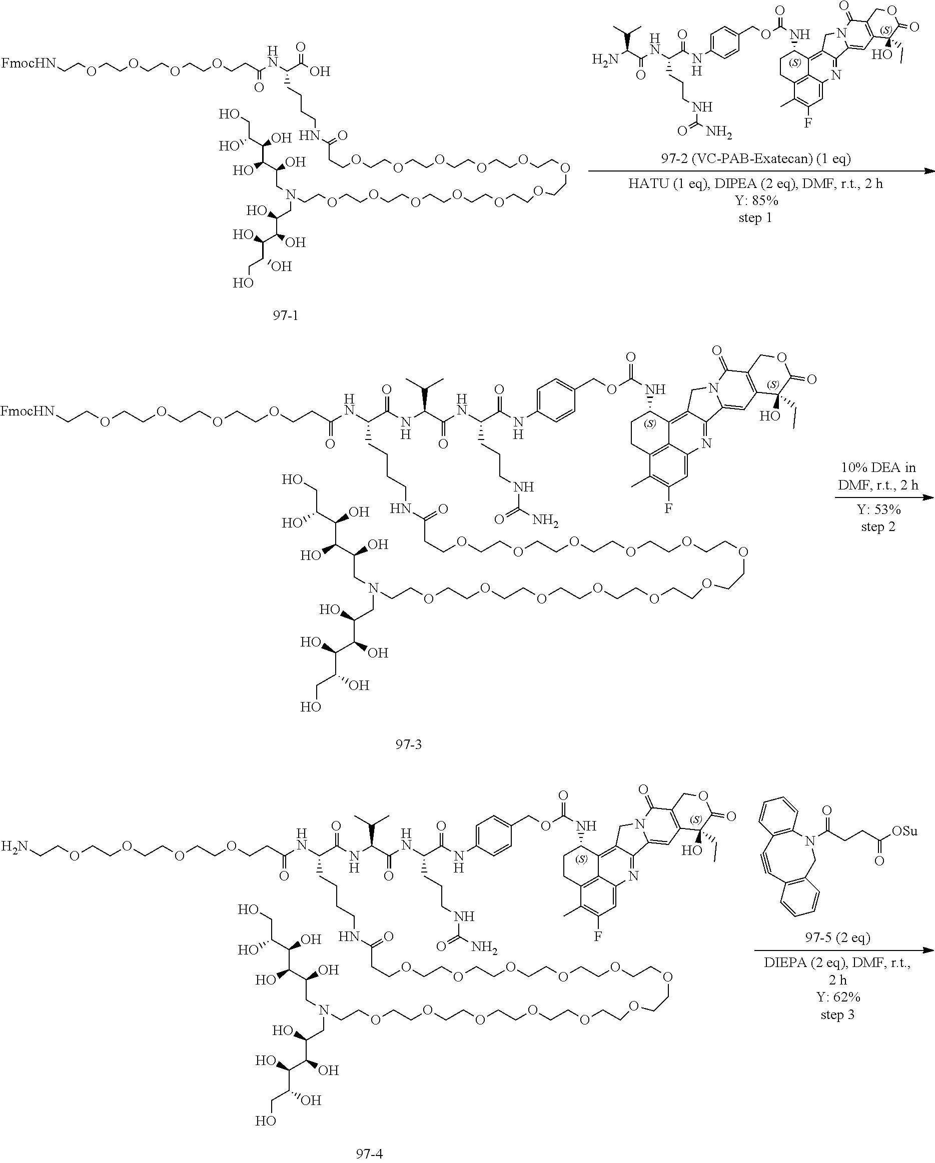

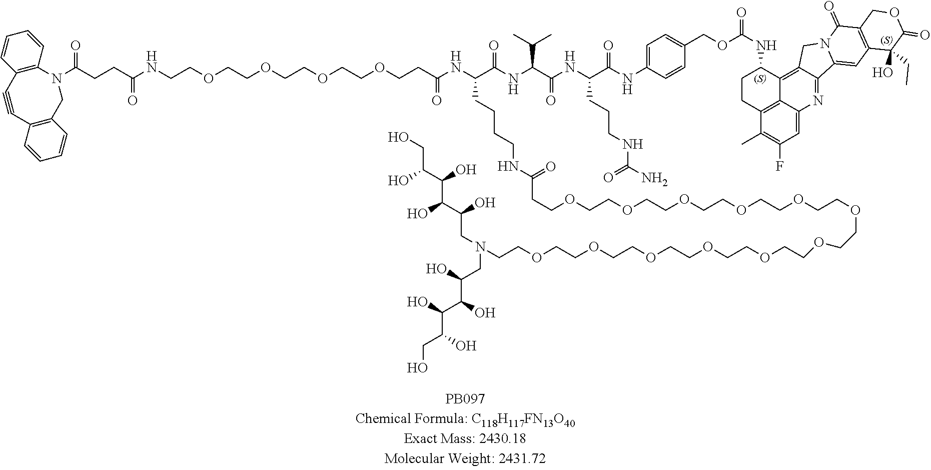

A Drug-Linker containing a PEG unit and a cleavable linker attached to exatecan (PB097) was prepared as follows:

### Step 1

A solution of compound 97-1 ((2S)-2-(1-{[(9H-fluoren-9-ylmethoxy)carbonyl]amino}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (97-1, 200 mg, 0.130 mmol)), compound 97-2 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0², ¹⁴0.0¹⁴, ¹³0.0⁶, ¹¹0.0²⁰,²⁴]tetracosa-1, 6(11),12,14,16,18,20(24)-heptaen-23-yl]carbamate (97-2, 108.83 mg, 0.129 mmol)) and HATU (49.43 mg, 0.130 mmol), DIPEA (33.59 mg, 0.260 mmol) in DMF (2 mL) was stirred at room temperature for 2 h and LCMS showed that the reaction was completed. The reaction mixture was then purified by reverse phase flash chromatography (0.01% TFA) to get the desired fractions, which were freeze-dried to yield compound 97-3 (9H-fluoren-9-ylmethyl N-(14-{[(1S)-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0², ¹⁴0.0⁴, ¹³0.0⁶, ¹³0.0²⁰,²⁴]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (97-3, 262 mg, 0.111 mmol, 85.16%)) as a pale yellow solid. ESI m/z: 789.7 (M/3+H)⁺.

### Step 2

To a solution of compound 97-3 ((9H-fluoren-9-yl)methyl N-(14-{[(1S)-1-{[(1S)-1-{[(1 S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamate (97-3, 162 mg, 0.068 mmol)) in DMF (2 mL) was added diethyl amine (0.2 mL, 0.2720 mmol). The mixture was stirred at room temperature for 2 hours. Then the crude mixture was purified by reverse phase flash chromatography (0.01% TFA) to get the desired fractions, which were freeze-dried to yield compound 97-4 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (97-4, 78 mg, 0.036 mmol, 53.49%)) as a white solid. ESI m/z: 715.7(M/3+H)⁺.

### Step 3

A solution of compound 97-4 ({4-[(2S)-2-[(2S)-2-[(2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (97-4, 78 mg, 0.036 mmol)), compound 97-5 (2,5-dioxopyrrolidin-1-yl 4-{2-azatricyclo[10.4.0.0^{4,9}]hexadeca-1(12),4(9),5,7,13,15-hexaen-10-yn-2-yl}-4-oxobutanoate (97-5, 29.27 mg, 0.073 mmol) and DIPEA (9.40 mg, 0.073 mmol)) in DMF (2 mL) was stirred at room temperature for 2 h to achieve complete conversion. Then the reaction mixture was purified by Prep-HPLC (10 mM NH₄HCO₃) to get the desired fractions, which were lyophilized by LabConco to yield PB097 ({4-[(2S)-2-[(2S)-2-[(2S)-2-[1-(4-{2-azatricyclo[10.4.0.0{4,9}]hexadeca-1(12),4(9),5,7,13,15-hexaen-10-yn-2-yl}-4-oxobutanamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB097, 55 mg, 0.023 mmol, 62.18%)) as a white solid. ESI m/z: 811.5 (M/3+H)⁺. [Note: product was partly cleaved on amide bond and DBCO fragment was released in acidic LCMS, so the DBCO unit was detected with m/z 206 and corresponding counterpart with m/z 749 ((M-205+17)/3+H)⁺ was detected. In the case of basic LCMS, the exatecan lactone ring was partly opened and fragment ion detected with m/z 817 ((M+18)/3+H)⁺]. ¹H NMR (400 MHz, DMSO-d6) δ 10.04 (s, 1H), 8.13 (d, J=8.0 Hz, 1H), 8.09-8.02 (m, 2H), 7.84-7.76 (m, 3H), 7.74-7.66 (m, 2H), 7.62-7.59 (m, 3H), 7.52-7.42 (m, 3H), 7.40-7.27 (m, 6H), 6.54 (s, 1H), 5.99 (t, J=6.4 Hz, 1H), 5.45-5.43 (m, 4H), 5.34-5.23 (m, 3H), 5.08 (s, 2H), 5.02 (d, J=14.0 Hz, 1H), 4.60-4.15 (m, 12H), 3.71-3.67 (m, 1H), 3.64-3.55 (m, 9H), 3.50-3.40 (m, 62H), 3.40-3.39 (m, 4H), 3.32-3.18 (m, 4H), 3.18-2.90 (m, 8H), 2.60-2.51 (m, 1H), 2.42-2.36 (m, 6H), 2.29 (t, J=6.0 Hz, 2H), 2.24-2.09 (m, 4H), 2.03-1.93 (m, 2H), 1.91-1.80 (m, 2H), 1.80-1.52 (m, 5H), 1.49-1.19 (m, 8H), 0.89-0.80 (m, 9H) ppm.

### Example 29: Preparation of a Drug-Linker Containing a PEG Unit and a Cleavable Linker Attached to MMAE (PB098; SEQ ID NO: 57). Compound 98-7 is Disclosed as SEQ ID NO: 73, and Compound 98-9 is Disclosed as SEQ ID NO: 69

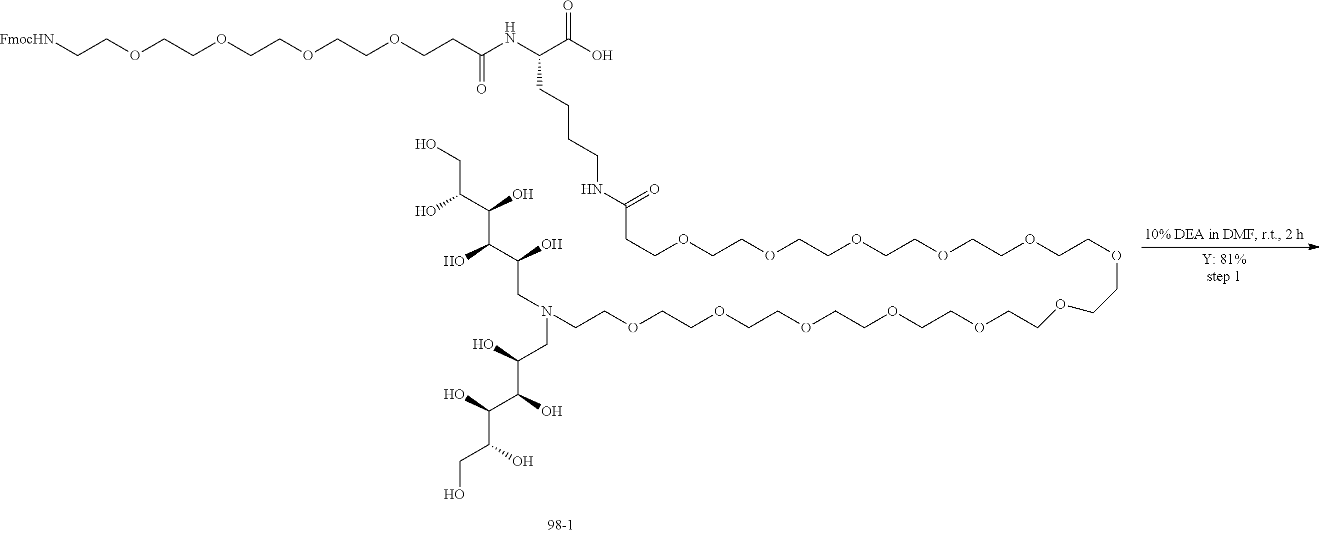

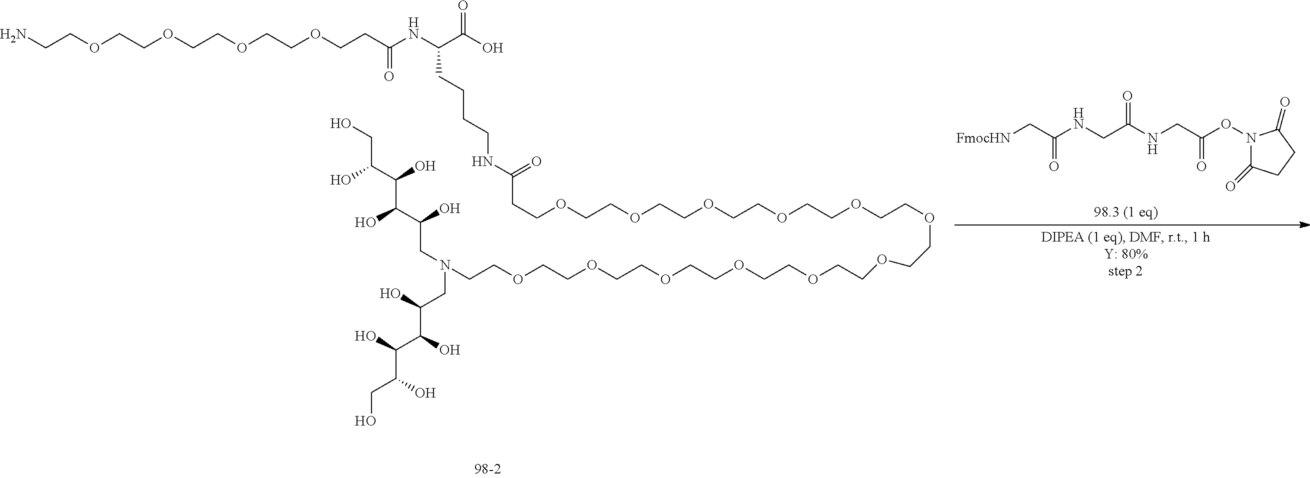

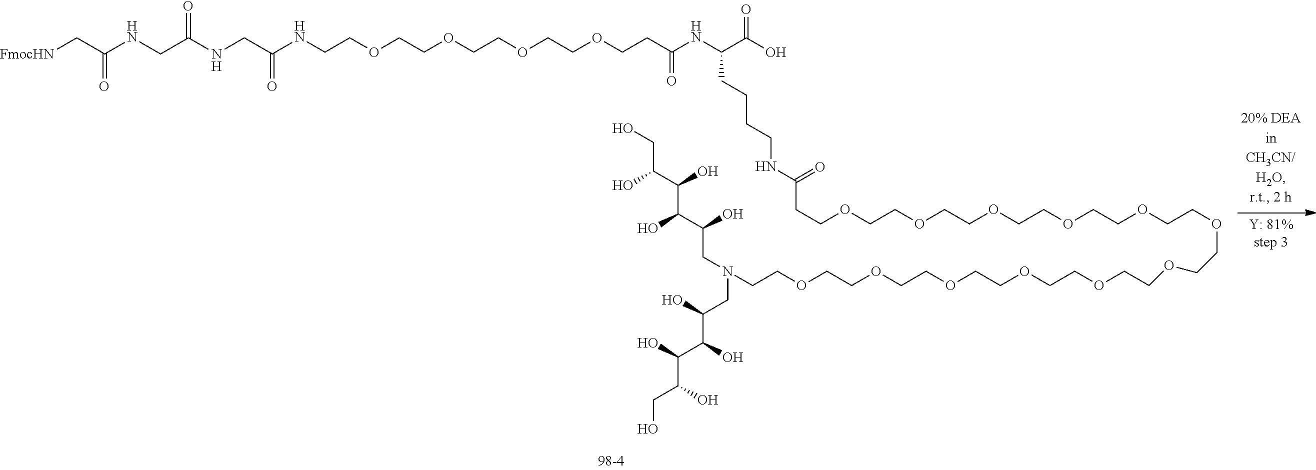

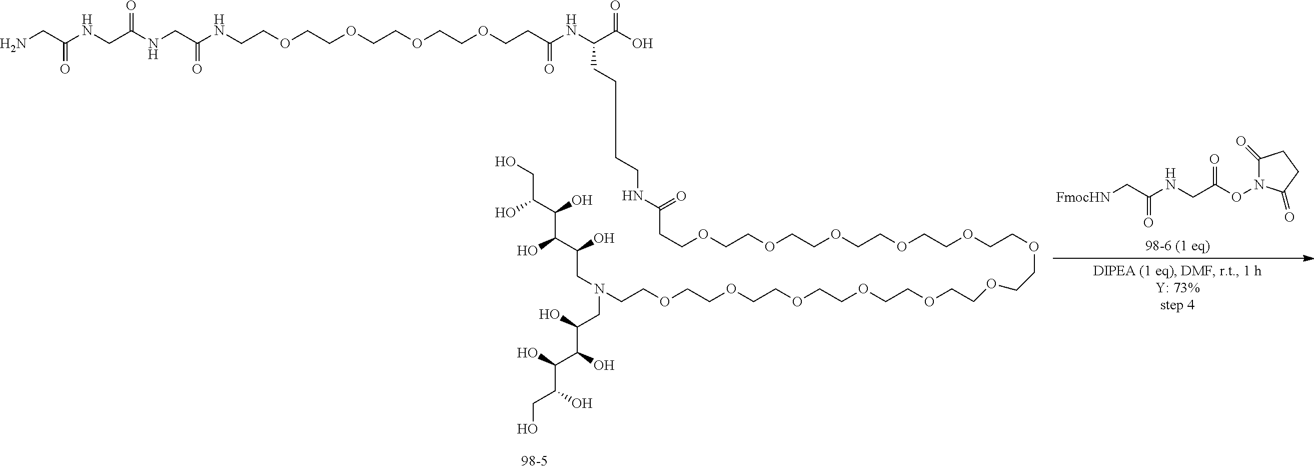

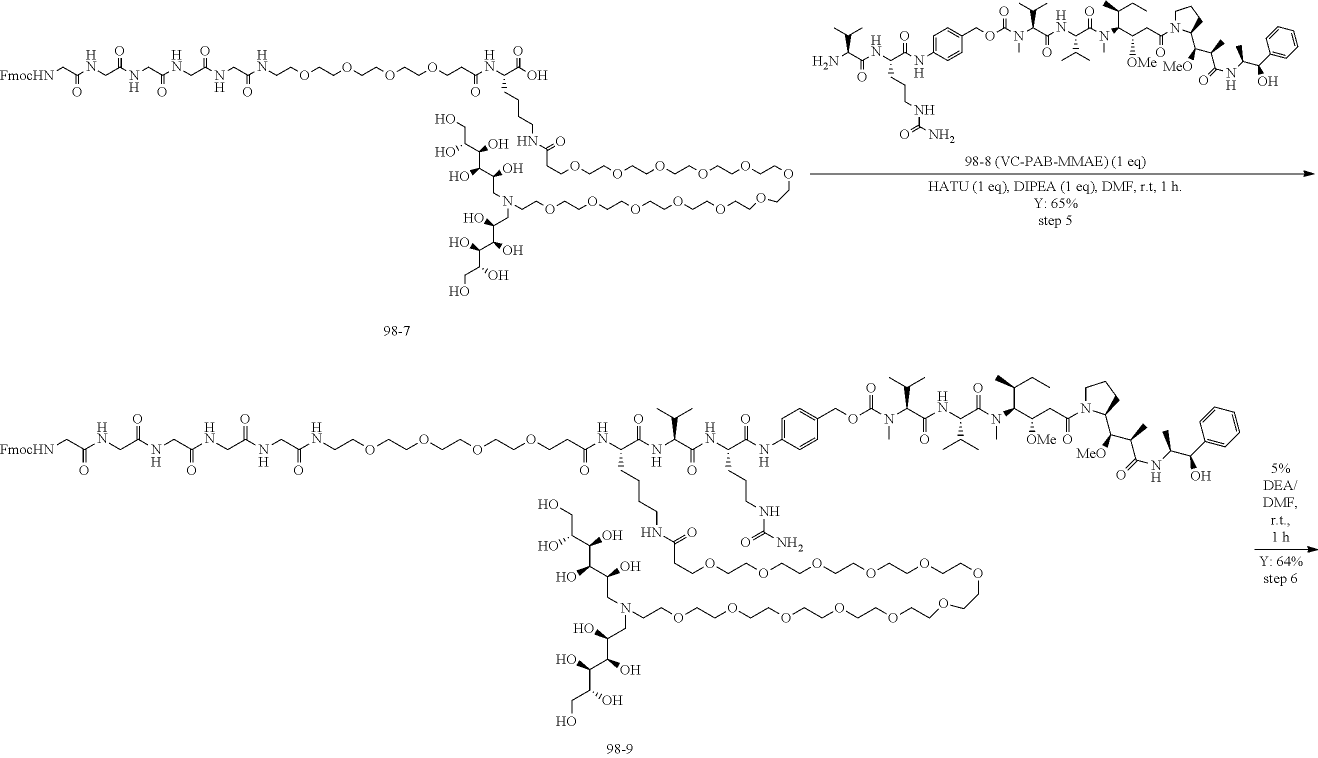

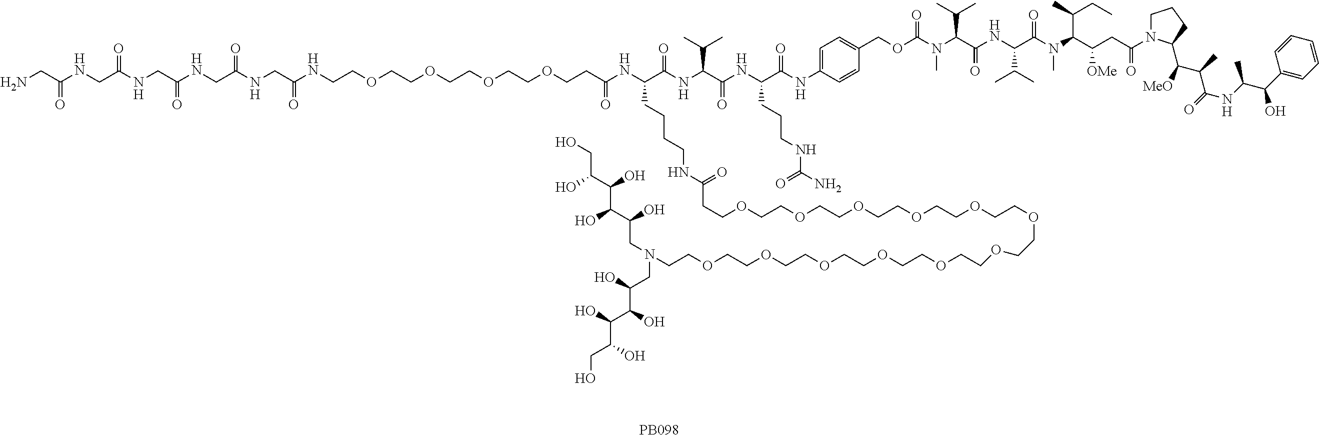

A Drug-Linker containing a PEG unit and a cleavable linker attached to MMAE (PB098) was prepared as follows:

### Step 1

To a solution of compound 98-1 ((2S)-2-[1-({[(9H-fluoren-9-yl)methoxy]carbonyl}amino)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-1, 3.0 g, 1.943 mmol)) in DMF (10 mL) was added diethyl amine (1.1 mL, 14.032 mmol). The mixture was stirred at room temperature for 2h to achieve complete deprotection. Then the resulting solution was purified directly by reverse phase flash chromatography (0.01% TFA) to afford the product 98-2 ((2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-2, 2.1 g, 1.589 mmol, 81.71%)) as white solid. ESI m/z: 661.6 (M/2+H)⁺.

### Step 2

To asolution of compound 98-2 ((2S)-2-(1-amino-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-2, 2.1 g, 1.589 mmol)) in DMF (10 mL) was added compound 98-3 (pentafluorophenyl 2-{2-[2-({[(9H-fluoren-9-yl)methoxy]carbonyl}amino)acetamido]acetamido}acetate (98-3, 0.92 g, 1.589 mmol)) and DIPEA (0.21 g, 1.589 mmol). The mixture was stirred at room temperature for 1h. The resulting solution was purified by reverse phase flash chromatography (0.01% TFA) to afford the product 98-4 ((2S)-2-[1-(2-{2-[2-({[(9H-fluoren-9-yl)methoxy]carbonyl}amino)acetamido]acetamido}acetamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-4, 2.2 g, 1.283 mmol, 80.59%)) as a white solid. ESI m/z: 858.2(M/2+H)⁺.

### Step 3

To the solution of compound 98-4 ((2S)-2-[1-(2-{2-[2-({[(9H-fluoren-9-yl)methoxy]carbonyl}amino)acetamido]acetamido}acetamido)-3,6,9,12-tetraoxapentadecan-15-amido]-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-4, 2.2 g, 1.283 mmol)) in MeCN (10 mL) and H₂O (6 mL) was added diethyl amine (4 mL, 2.840 mmol). The mixture was stirred at room temperature for 2h to completion. The resulting solution was concentrated to dryness and purified by reverse phase flash chromatography (0.01% TFA) to afford the product 98-5 ((2S)-2-(1-{2-[2-(2-aminoacetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-5, 1.55 g, 1.038 mmol, 81.15%)) as white gel. ESI m/z: 747.1 (M/2+H)⁺, 498.5 (M/3+H)⁺.

### Step 4

To a solution of compound 98-5 ((2S)-2-(1-{2-[2-(2-aminoacetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-5, 1.55 g, 1.038 mmol)) in DMF (10 mL) was added compound 98-6 (2,5-dioxopyrrolidin-1-yl 2-[2-({[(9H-fluoren-9-yl)methoxy]carbonyl}amino)acetamido]acetate (98-6, 0.47 g, 1.038 mmol)) and DIPEA (0.13 g, 1.038 mmol). The mixture was stirred at room temperature for 1h. The resulting solution was purified by reverse phase flash chromatography (0.01% TFA) to afford the product 98-7 ((2S)-2-(1-{2-[2-(2-{2-[2-({[(9H-fluoren-9-yl)methoxy]carbonyl}amino)acetamido]acetamido}acetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-7, 1.4 g, 0.765 mmol, 73.68%)) as a white solid. ESI m/z: 915.2 (M/2+H)⁺, 610.7 (M/3+H)⁺. ¹H NMR (400 MHz, DMSO-d6) δ 8.22-8.15 (m, 4H), 8.11-8.08 (m, 2H), 7.91-7.86 (m, 3H), 7.82 (t, J=5.6 Hz, 1H), 7.72 (d, J=7.6 Hz, 2H), 7.59 (t, J=6.0 Hz, 1H), 7.44-7.40 (m, 2H), 67.36-7.31 (m, 2H), 4.55-4.12 (m, 10H), 3.80-3.72 (m, 8H), 3.69-3.66 (m, 4H), 3.62-3.56 (m, 10H), 3.53-3.47 (m, 60H), 3.42-3.38 (m, 5H), 3.32-3.19 (m, 2H), 3.03-2.92 (m, 8H), 2.50-2.36 (m, 2H), 2.29 (t, J=6.4 Hz, 2H), 1.72-1.63 (m, 1H), 1.59-1.50 (m, 1H), 11.41-1.35 (m, 2H), 1.31-1.16 (m, 5H) ppm.

### Step 5

To the solution of compound 98-7 ((2S)-2-(1-{2-[2-(2-{2-[2-({[(9H-fluoren-9-yl)methoxy]carbonyl}amino)acetamido]acetamido}acetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (98-7, 200 mg, 0.109 mmol)) and compound 98-8 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (98-8, 122.85 mg, 0.109 mmol)) in dry DMF (5 mL) was added HATU (41.58 mg, 0.109 mmol) and DIPEA (14.13 mg, 0.109 mmol) sequentially. After addition, the mixture was stirred at room temperature for 1 h until all starting amine was consumed (monitored by LCMS). Then the resulting solution was purified by reverse phase flash chromatography (0.01% TFA) to get the desired fractions, which were lyophilized to yield the product 98-9 ((9H-fluoren-9-yl)methyl N-[({[({[({[(14-{[(1S)-1-{[(1S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)meth yl]carbamate (98-9, 210 mg, 0.072 mmol, 65.65%)) as white solid. ESI m/z: 979.0(M/3+H)⁺.

### Step 6

To a solution of compound 98-9 ((9H-fluoren-9-yl)methyl N-[({[({[({[(14-{[(1S)-1-{[(1S)-1-{[(1 S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)meth yl]carbamate (98-9, 210 mg, 0.072 mmol)) in anhydrous DMF (1.9 mL) was added diethyl amine (0.1 mL, 0.971 mmol). The solution was stirred at room temperature for 1h to completion. The resulting solution was purified directly by Prep-HPLC (0.01% TFA) to afford a TFA salt of product PB098 ({4-[(2S)-2-[(2S)-2-[(2S)-2-{1-[2-(2-{2-[2-(2-aminoacetamido)acetamido]acetamido}acetamido)acetamido]-3,6,9,12-tetraoxapentadecan-15-amido}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB098, 125 mg, 0.046 mmol, 64.40%)) as a white solid. ESI m/z: 905.0(M/3+H)⁺, retention time 5.432 min (HPLC). ¹H NMR (400 MHz, DMSO-d6) δ 10.05 (s, 1H), 8.64 (t, J=5.6 Hz, 1H), 8.36-8.01 (m, 9H), 7.93-7.91 (m, 1.5H), 7.84-7.81 (m, 1H), 7.73 (d, J=8.4 Hz, 0.5H), 7.58 (d, J=7.6 Hz, 2H), 7.35-7.24 (m, 6H), 7.20-7.14 (m, 1H), 6.05-5.96 (m, 1H), 5.44 (brs, 4H), 5.13-4.94 (m, 2H), 4.82-4.38 (m, 10H), 4.30-4.17 (m, 4H), 4.04-3.93 (m, 4H), 3.86 (d, J=5.6 Hz, 2H), 3.78-3.74 (m, 7H), 3.69-3.67 (m, 5H), 3.62-3.55 (m, 14H), 3.55-3.39 (m, 56H), 3.39-3.30 (m, 5H), 3.25-3.04 (m, 11H), 3.05-2.83 (m, 10H), 2.41-2.36 (m, 3H), 2.33-2.27 (m, 3H), 2.16-1.93 (m, 4H), 1.81-1.21 (m, 18H), 1.05-0.97 (m, 6H), 0.89-0.73 (m, 27H) ppm. One proton of carboxyl group on TFA was revealed.

### Example 30: Preparation of a Drug-Linker Containing a PEG Unit and a Cleavable Linker Attached to Exatecan (PB099; SEQ ID NO: 58). Compound 99-1 is Disclosed as SEQ ID NO: 73, and Compound 99-3 is Disclosed as SEQ ID NO: 68

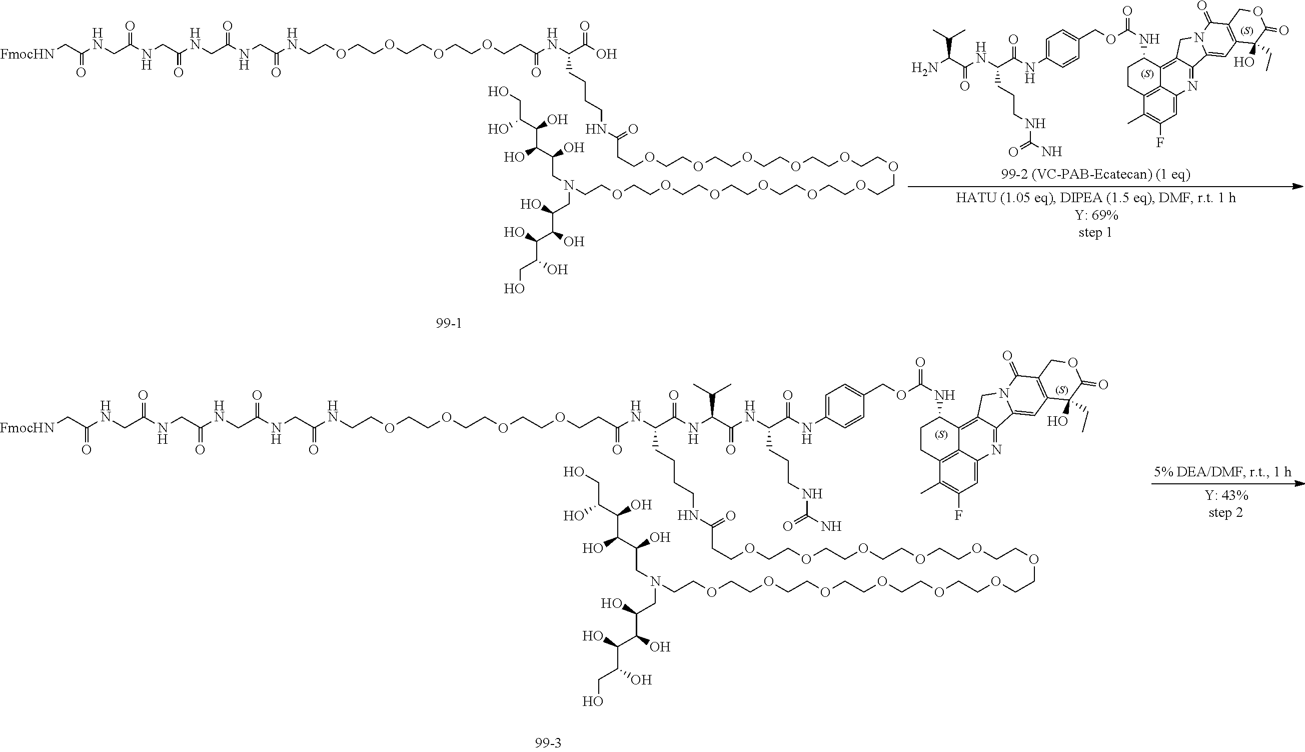

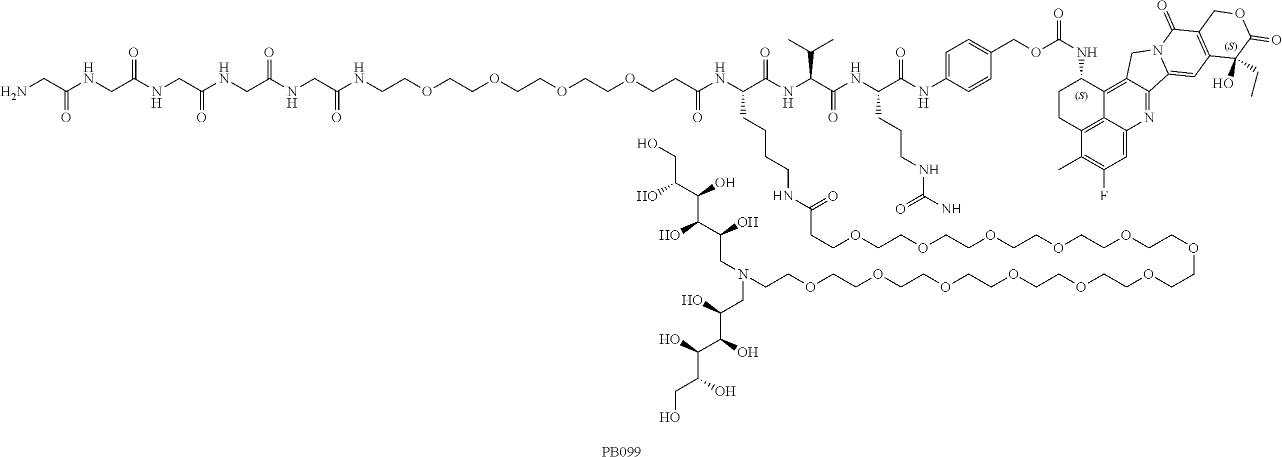

A Drug-Linker containing a PEG unit and a cleavable linker attached to exatecan (PB099) was prepared as follows:

### Step 1

To a solution of compound 99-1 ((2S)-2-(1-{2-[2-(2-{2-[2-({[(9H-fluoren-9-yl)methoxy]carbonyl}amino)acetamido]acetamido}acetamido)acetamido]acetamido}-3,6,9,12-tetraoxapentadecan-15-amido)-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanoic acid (99-1, 190 mg, 0.104 mmol)) in DMF (4 mL) was added compound 99-2 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (99-2, 88 mg, 0.105 mmol)), HATU (41.47 mg, 0.109 mmol) and DIPEA (0.207 mL, 0.156 mmol). The mixture was stirred at room temperature for 1h to complete (monitored by LCMS). Then resulting solution was purified by reverse phase flash chromatography (0.01% TFA) to afford the product 99-3 ((9H-fluoren-9-yl)methyl N-[({[({[({[(14-{[(1S)-1-{[(1 S)-1-{[(1 S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)meth yl]carbamate (99-3, 190 mg, 0.072 mmol, 68.97%)) as a yellow solid. ESI m/z: 885.0(M/3+H)⁺,663.9(M/4+H)⁺.

### Step 2

To the solution of compound 99-3 ((9H-fluoren-9-yl)methyl N-[({[({[({[(14-{[(1S)-1-{[(1 S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-5-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentyl]carbamoyl}-3,6,9,12-tetraoxatetradecan-1-yl)carbamoyl]methyl}carbamoyl)methyl]carbamoyl}methyl)carbamoyl]methyl}carbamoyl)meth yl]carbamate (99-3, 102 mg, 0.038 mmol)) in DMF (1.9 mL) was added diethyl amine (0.1 mL, 0.971 mmol). The mixture was stirred at room temperature for 1 h to completion. The resulting solution was purified by reverse phase separation (0.01% TFA) to afford a TFA salt of product PB099 ({4-[(2S)-2-[(2S)-2-[(2S)-2-{1-[2-(2-{2-[2-(2-aminoacetamido)acetamido]acetamido}acetamido)acetamido]-3,6,9,12-tetraoxapentadecan-15-amido}-6-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB099, 40 mg, 0.016 mmol, 42.80%)) as a yellow solid. ESI m/z: 810.8 (M/3+H)⁺, 608.3 (M/4+H)⁺, retention time 5.077 min (HPLC).

¹H NMR (400 MHz, DMSO-d6) δ 10.05 (s, 1H), 8.64 (t, J=5.6 Hz, 1H), 8.32 (t, J=5.6 Hz, 1H), 8.21-8.01 (m, 9H), 7.92 (t, J=5.6 Hz, 1H), 7.85-7.72 (m, 3H), 7.60 (d, J=8.4 Hz, 2H), 7.36 (d, J=8.4 Hz, 2H), 7.31 (s, 1H), 6.55 (s, 1H), 6.02 (t, J=5.6 Hz, 1H), 5.46 (brs, 6H), 5.34-5.23 (m, 3H), 5.08 (s, 2H), 4.83 (brs, 2H), 4.56 (brs, 4H), 4.41-4.25 (m, 3H), 4.21-4.17 (m, 1H), 3.99 (brs, 2H), 3.86 (d, J=5.2 Hz, 2H), 3.78-3.73 (m, 6H), 3.69-3.67 (m, 4H), 3.62-3.54 (m, 14H), 3.52-3.47 (m, 60H), 3.29-3.20 (m, 6H), 3.14-2.90 (m, 6H), 2.41-2.37 (m, 5H), 2.29 (t, J=6.4 Hz, 2H), 2.24-2.13 (m, 2H), 1.99-1.81 (m, 3H), 1.70-1.58 (m, 3H), 1.50-1.20 (m, 8H), 0.90-0.81 (m, 9H) ppm. Two proton of carboxyl group on TFA were revealed.

### Example 31: Preparation of a Drug-Linker Containing a PEG Unit and a Cleavable Linker Attached to Exatecan (PB100 or “LD100”)

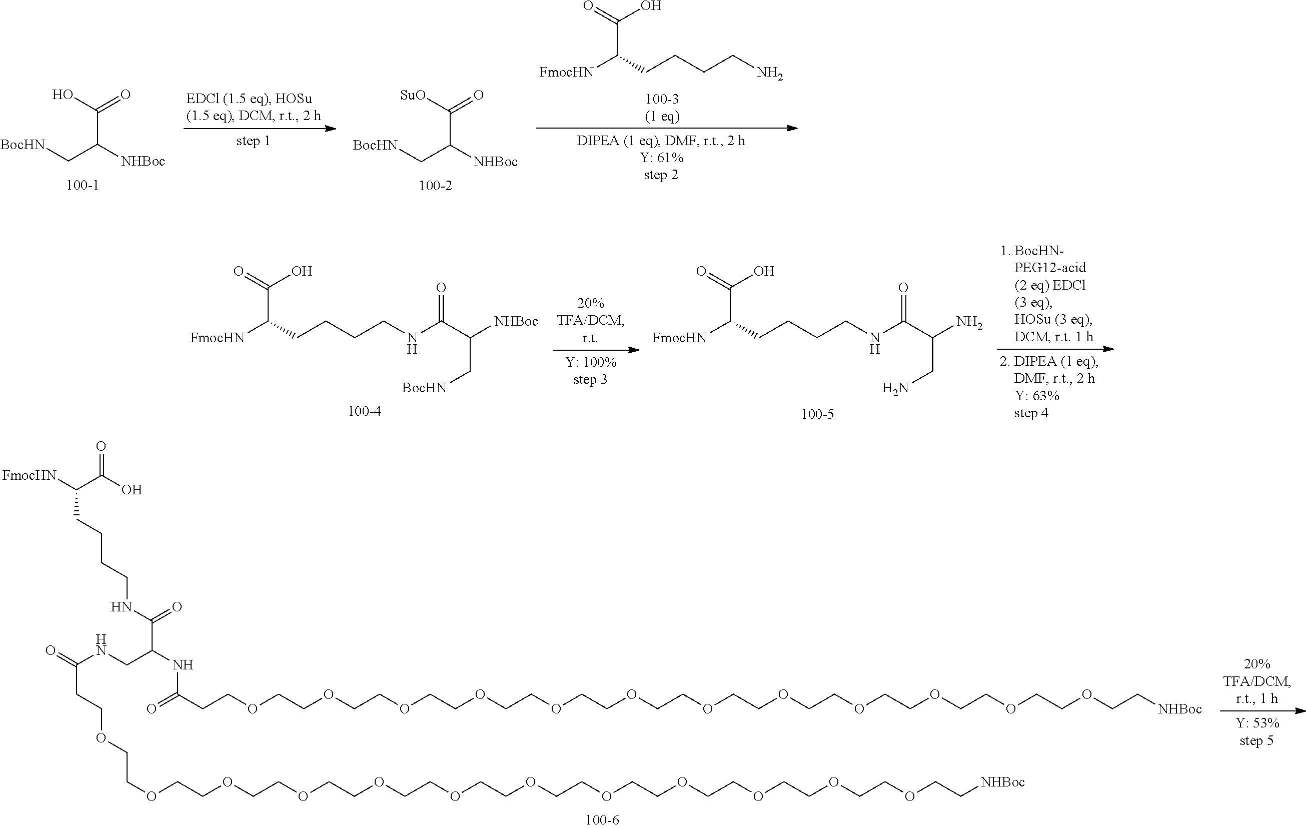

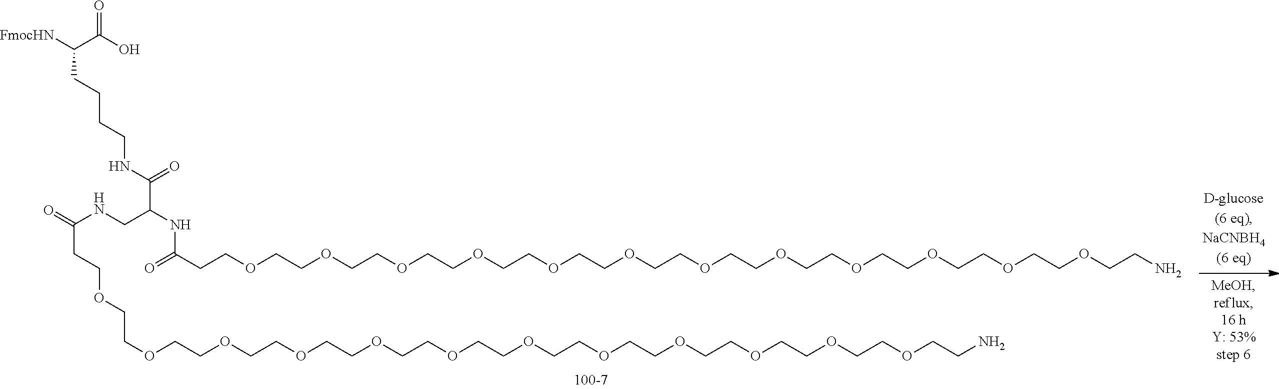

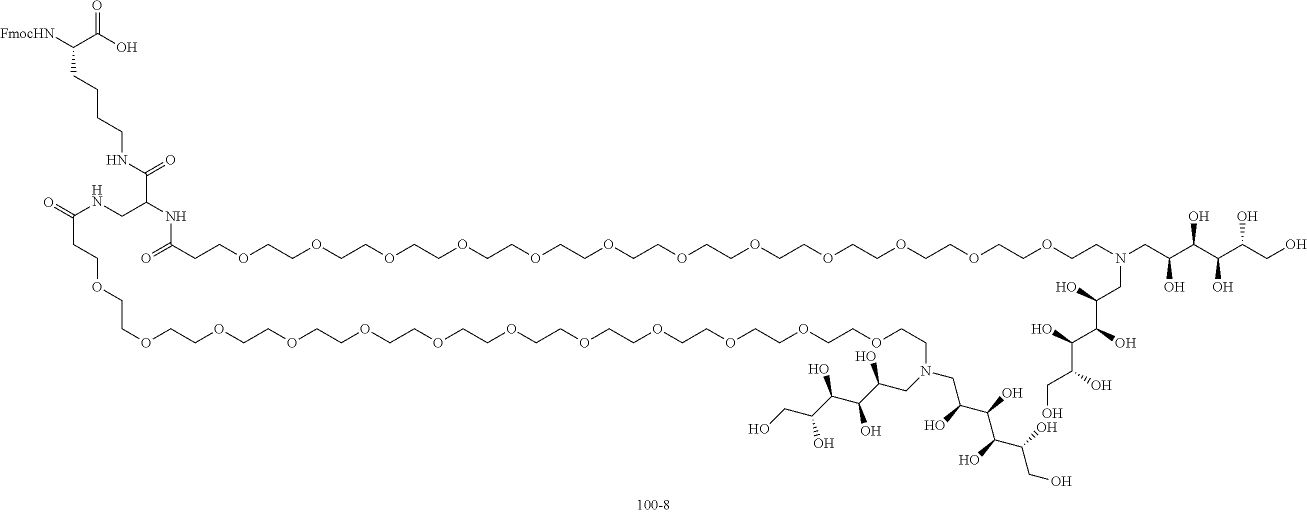

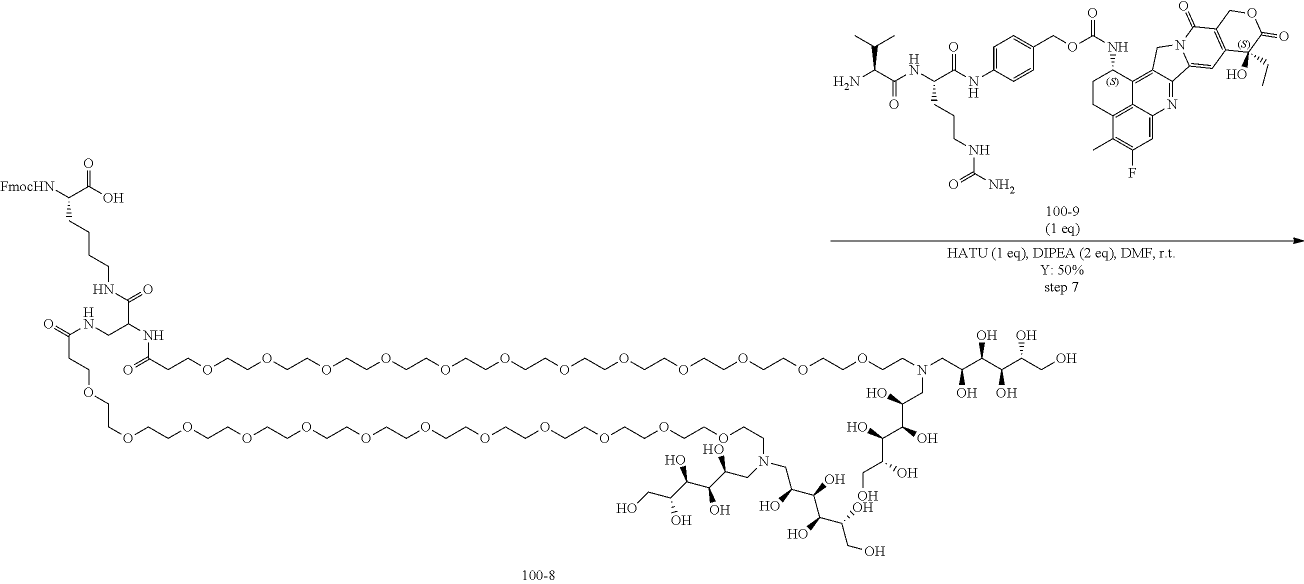

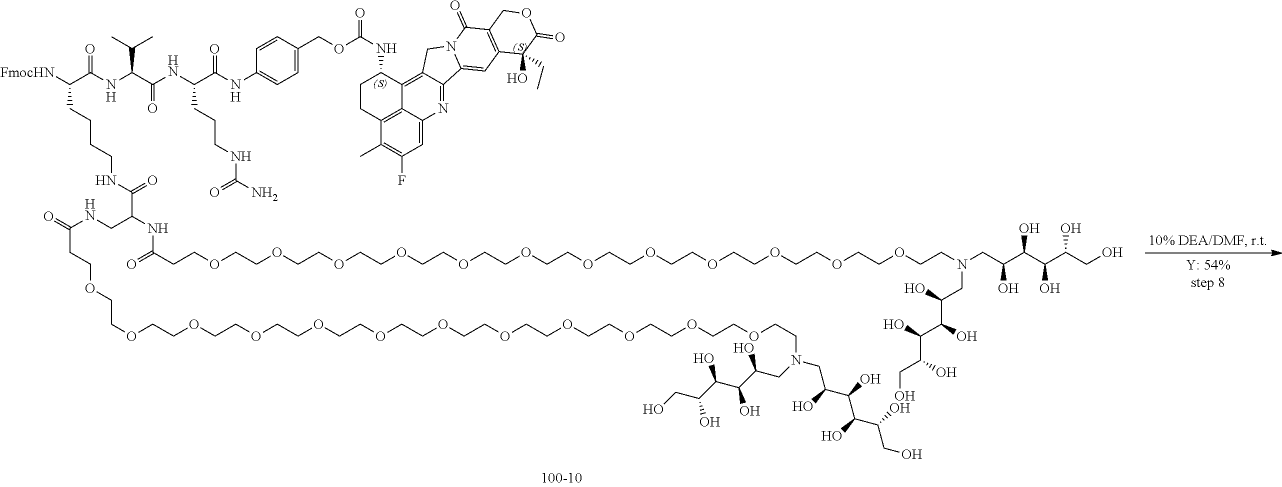

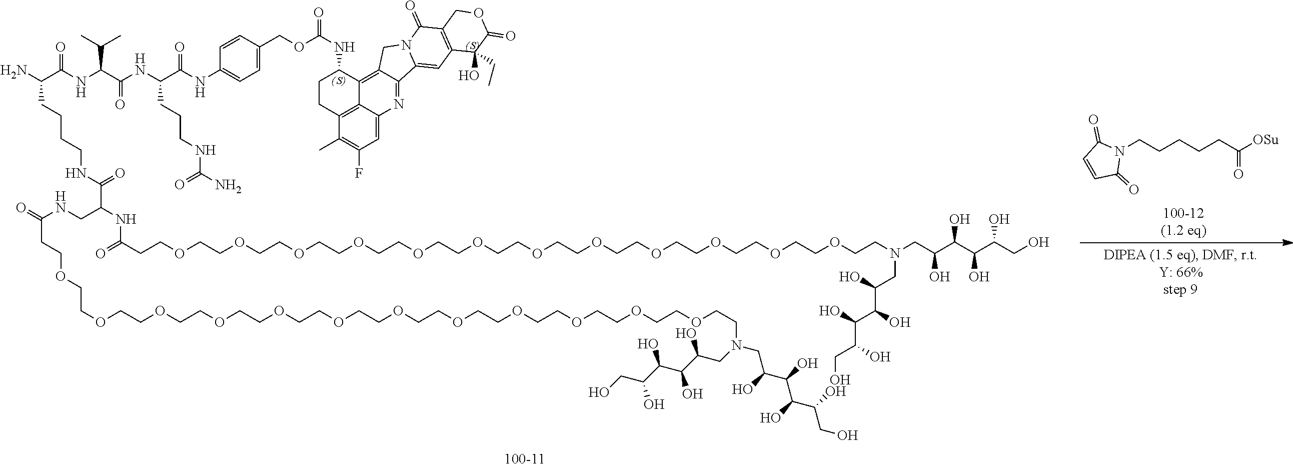

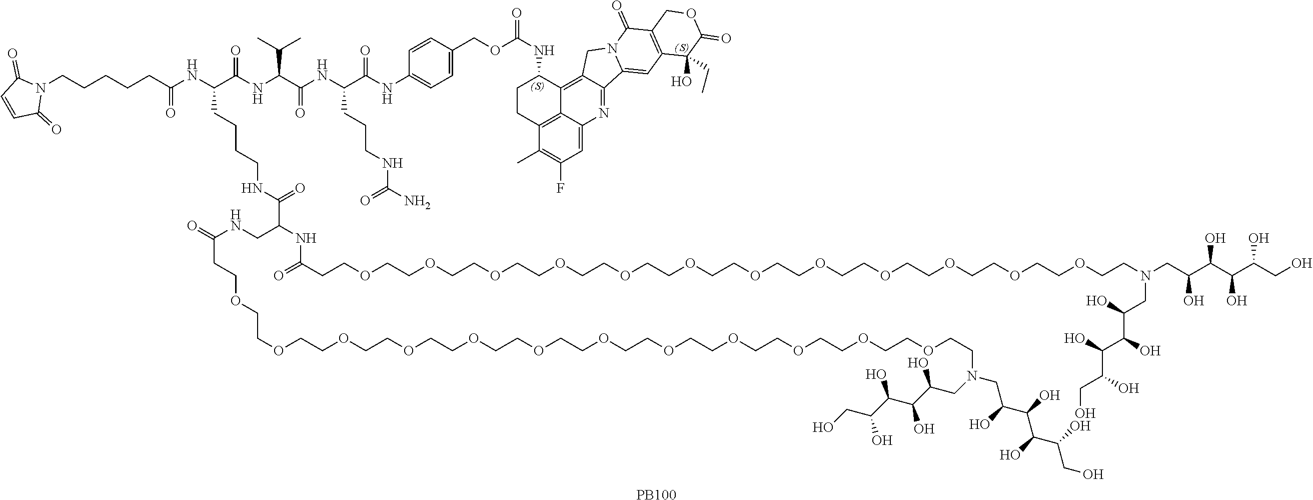

A Drug-Linker containing a PEG unit and a cleavable linker attached to exatecan (PB100 or LD100) was prepared as follows:

### Step 1

A solution of compound 100-1 (2,3-bis({[(tert-butoxy)carbonyl]amino})propanoic acid (100-1, 1.52 g, 5.000 mmol)) and HOSu (0.86 g, 7.500 mmol) in anhydrous DCM (20 mL) was stirred at room temperature, then EDCl (1.44 g, 7.500 mmol) was added portionwise over 5 min. The resulting solution was stirred for another 1h until the starting acid was consumed (monitored by LCMS). Then the reaction solution was diluted with more DCM (20 mL) and washed with water (40 mL), the organic layer was separated and the water phase was extracted with more DCM (2*40 mL). The collected DCM layers were dried over sodium sulfate, filtered and concentrated the filtrate to give crude compound 100-2 (2,5-dioxopyrrolidin-1-yl 2,3-bis((tert-butoxycarbonyl)amino)propanoate (100-2, 2.0 g, 4.988 mmol, 99.75%)) as a white foam, which turned into colorless oil after standing for minutes. The compound was used as such in the next step. ESI m/z: 424.2 (M+Na)⁺.

### Step 2

A solution of compound 100-3 ((2S)-6-amino-2-({[(9H-fluoren-9-yl)methoxy]carbonyl}amino)hexanoic acid (100-3, 1.84 g, 4.988 mmol)) and DIPEA (0.64 g, 4.988 mmol) in dry DMF (10 mL) was stirred at room temperature for 10 min, then a solution of compound 100-2 (2,5-dioxopyrrolidin-1-yl 2,3-bis({[(tert-butoxy)carbonyl]amino})propanoate (100-2, 2.0 g, 4.988 mmol)) in anhydrous DMF (10 mL) was added slowly over 5 min. After addition, the resulting suspension was stirred for 2 h until all starting materials were consumed. The resulting solution was purified directly by reverse phase flash chromatography (0.01% TFA) to yield compound 100-4 ((2S)-6-[2,3-bis({[(tert-butoxy)carbonyl]amino})propanamido]-2-({[(9H-fluoren-9-yl)methoxy]carbonyl}amino)hexanoic acid (100-4, 2.0 g, 3.055 mmol, 61.16%)) as a white solid. ESI m/z: 555.3 (M-100+H)⁺, 678.4 (M+Na)⁺. ¹H NMR (400 MHz, DMSO-d6) δ 12.55 (s, 1H), 7.90 (d, J=7.6 Hz, 2H), 7.84 (t, J=4.8 Hz, 1H), 7.73 (d, J=7.2 Hz, 2H), 7.62 (d, J=8.0 Hz, 1H), 7.44-7.40 (m, 2H), 7.36-7.31 (m, 2H), 6.73 (m, 1H), 6.61 (d, J=8.0 Hz, 1H), 4.29-4.21 (m, 3H), 3.98-3.87 (m, 2H), 3.21-3.16 (m, 2H), 3.11-2.94 (m, 2H), 1.74-1.55 (m, 2H), 1.39-1.26 (m, 22H) ppm.

### Step 3

A solution of compound 100-4 ((2S)-6-[2,3-bis({[(tert-butoxy)carbonyl]amino})propanamido]-2-({[(9H-fluoren-9-yl)methoxy]carbonyl}amino)hexanoic acid (100-4, 1.0 g, 1.527 mmol)) in DCM (8 mL) was stirred at room temperature, then TFA (2 mL, 26.925 mmol) was added slowly. The solution was stirred for another 1h, then the solution was evaporated to dryness. The residue was dissolved with DCM (20 mL) again and concentrated. The process was repeated twice to yield crude product 100-5 ((2S)-6-(2,3-diaminopropanamido)-2-({[(9H-fluoren-9-yl)methoxy]carbonyl}amino)hexanoic acid (100-5, 1.0 g, 2.200 mmol, 144.05%)) as pale yellow oil, used as such in the next step. ESI m/z: 455.3 (M+H)⁺.

### Step 4

A solution of 1-{[(tert-butoxy)carbonyl]amino}-3,6,9,12,15,18,21,24,27,30,33,36-dodecaoxanonatriacontan-39-oic acid (2.2 g, 3.065 mmol) and HOSu (0.53 g, 4.597 mmol) in anhydrous DCM (10 mL) was stirred at room temperature for 5 min, then EDCl (0.88 g, 4.597 mmol) was added. The resulting solution was stirred for another 1h until all acid was converted into activated ester. Then the solution was diluted with more DCM (20 mL) and washed with water (20 mL), the organic layer was collected and the water layer was extracted with more DCM (20 mL*2). The DCM layer was combined and dried over sodium sulfate, filtered and concentrated the filtrate to give crude active ester as colorless oil. The activated ester was dissolved in anhydrous DMF (5 mL), and added to a solution of compound 100-5 ((2S)-6-(2,3-diaminopropanamido)-2-({[(9H-fluoren-9-yl)methoxy]carbonyl}amino)hexanoic acid (100-5, 0.63 g, 1.390 mmol)) and DIPEA (0.36 g, 2.780 mmol) in anhydrous DMF (5 mL) slowly. After addition, the resulting pale yellow solution was stirred at room temperature for 2h to completion. Then the reaction solution was purified by reverse phase flash chromatography (0.01% TFA) to yield compound 100-6 as a colorless oil ((2S)-6-[2,3-bis(1-{[(tert-butoxy)carbonyl]amino}-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-6)). ESI m/z: 551.3 ((M-100)/3+H)⁺, 827.7 (M-100)/2+H)⁺.

### Step 5

A solution of compound 100-6 ((2S)-6-[2,3-bis(1-{[(tert-butoxy)carbonyl]amino}-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-6, 1.8 g, 0.971 mmol)) in DCM (8 mL) was stirred at room temperature, then TFA (2 mL, 26.925 mmol) was added. The resulting solution was stirred for 1 h. Then the solution was concentrated to dryness and the residue was purified by reverse phase flash chromatography (0.01% TFA) to give the expected fractions, which were lyophilized to yield product 100-7 ((2S)-6-[2,3-bis(1-amino-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-7, 1.0 g, 0.605 mmol, 62.28%)) as colorless oil. ESI m/z: 552.3 (M/3+H)⁺, 828.1 (M/2+H)⁺. ¹H NMR (400 MHz, DMSO-d6) δ 7.95-7.89 (m, 3H), 7.85-7.72 (m, 9H), 7.63 (d, J=8.0 Hz, 1H), 7.45-7.41 (m, 2H), 7.36-7.31 (m, 2H), 4.29-4.20 (m, 4H), 3.94-3.88 (m, 1H), 3.68-3.66 (m, 1H), 3.61-3.57 (m, 18H), 3.57-3.47 (m, 76H), 3.34-3.29 (m, 2H), 3.26-3.21 (m, 1H), 3.05-2.97 (m, 6H), 2.39 (t, J=6.8 Hz, 2H), 2.31 (t, J=6.8 Hz, 2H), 1.74-1.55 (m, 2H), 1.44-1.25 (m 4H) ppm.

### Step 6

A suspension of compound 100-7 ((2S)-6-[2,3-bis(1-amino-3,6,9,12,15,18,21,24,27,30,33,36-dodecaoxanonatriacontan-39-amido)propanamido]-2-({[(9H-fluoren-9-yl)methoxy]carbonyl}amino)hexanoic acid (100-7, 850 mg, 0.514 mmol)) and D-glucose (555.03 mg, 3.084 mmol) in methanol (20 mL) was heated to 50° C. under nitrogen atmosphere and then sodium cyanoborohydride (193.77 mg, 3.084 mmol) was added. The resulting mixture was heated overnight (18 h) and completed. Then the solution was concentrated to dryness, the residue was dissolved in DMF and purified by reverse phase flash chromatography (0.01% TFA) to yield compound 100-8 ((2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-({[(9H-fluoren-9-yl)methoxy]carbonyl}amino)hexanoic acid (100-8, 640 mg, 0.173 mmol, 53.9%)) as colorless oil. ESI m/z=771.0 (M/3+H)⁺. ¹H NMR (400 MHz, DMSO-d6) δ 12.47 (s, 1H), 8.16 (brs, 2H), 7.95-7.80 (m, 5H), 7.73 (d, J=7.6 Hz, 2H), 7.63 (d, J=8.0 Hz, 1H), 7.45-7.40 (m, 2H), 7.35-7.31 (m, 2H), 5.48-5.41 (m, 4H), 4.85-4.76 (m, 4H), 4.64-4.41 (m, 12H), 4.31-4.20 (m, 4H), 4.04-3.95 (m, 4H), 3.95-3.87 (m, 1H), 3.79-3.76 (m, 5H), 3.72-3.61 (m, 5H), 3.59-3.55 (m, 20H), 3.55-3.40 (m, 98H), 3.29-3.21 (m, 2H), 3.11-2.97 (m, 2H), 2.38 (t, J=6.4 Hz, 2H), 2.31 (t, J=6.4 Hz, 2H), 1.74-1.56 (m, 2H), 1.43-1.26 (m, 4H) ppm. Two protons of carboxyl group from TFA were also revealed.

### Step 7

A solution of compound 100-8 ((2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-({[(9H-fluoren-9-yl)methoxy]carbonyl}amino)hexanoic acid (100-8, 150 mg, 0.065 mmol)), compound 100-9 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamate (100-9, 54.59 mg, 0.065 mmol)) and DIPEA (16.75 mg, 0.130 mmol) in anhydrous DMF (1.5 mL) was stirred at room temperature for 5 min to allow starting materials dissolve thoroughly in the solvent, then a solution of HATU (24.68 mg, 0.065 mmol) in anhydrous DMF (0.5 mL) was added. The resulting solution was stirred for another 2 h until LCMS indicated complete reaction. The completed reaction solution was purified directly by reverse phase flash chromatography (0.01% TFA) to yield compound 100-10 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-{[(1 S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (100-10, 103 mg, 0.033 mmol, 50.63%)) as a white solid. ESI m/z: 784.2 (M/4+H)⁺.

### Step 8

The solution of compound 100-10 (4-((2S,5S,8S,59S,60R,61R,62R)-8-((((9H-fluoren-9-yl)methoxy)carbonyl)amino)-59,60,61,62,63-pentahydroxy-5-isopropyl-4,7,14,18-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-57-((2S,3R,4R,5R)-2,3,4,5,6-pentahydroxyhexyl)-2-(3-ureidopropyl)-21,24,27,30,33,36,39,42,45,48,51,54-dodecaoxa-3,6,13,17,57-pentaazatrihexacontanamido)benzyl ((1S,9S)-9-ethyl-5-fluoro-9-hydroxy-4-methyl-10,13-dioxo-2,3,9,10,13,15-hexahydro-1 H,12H-benzo[de]pyrano[3′,4′:6,7]indolizino[1,2-b]quinolin-1-yl)carbamate (100-10, 100 mg, 0.032 mmol)) in DMF (1.8 mL) was stirred at room temperature and diethyl amine (0.2 mL, 1.941 mmol) was added. The reaction solution was stirred for 1 h and completed. Most of diethyl amine was evaporated with a rotary evaporator, then the residue was purified by reverse phase flash chromatography (0.01% TFA) to yield the expected product 100-11 (4-((2S,5S,8S,59S,60R,61R,62R)-8-amino-59,60,61,62,63-pentahydroxy-5-isopropyl-4,7,14,18-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-57-((2S,3R,4R,5R)-2,3,4,5,6-pentahydroxyhexyl)-2-(3-ureidopropyl)-21,24,27,30,33,36,39,42,45,48,51,54-dodecaoxa-3,6,13,17,57-pentaazatrihexacontanamido)benzyl ((1S,9S)-9-ethyl-5-fluoro-9-hydroxy-4-methyl-10,13-dioxo-2,3,9,10,13,15-hexahydro-1 H,12H-benzo[de]pyrano[3′,4′:6,7]indolizino[1,2-b]quinolin-1-yl)carbamate (100-11, 50 mg, 0.017 mmol, 53.82%)) as a pale yellow solid. ESI m/z: 971.5 (M/3+H)⁺, 728.9 (M/4+H)⁺.

### Step 9

A solution of compound 100-11 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (100-11, 50 mg, 0.017 mmol)) and DIPEA (3.32 mg, 0.026 mmol) in anhydrous DMF (1.5 mL) was stirred at room temperature for 5 min, then a solution of compound 100-12 (2,5-dioxopyrrolidin-1-yl 6-(2,5-dioxo-2,5-dihydro-1H-pyrrol-1-yl)hexanoate (100-12, 6.35 mg, 0.021 mmol)) in anhydrous DMF (0.5 mL) was added dropwise by syringe over 5 min. The resulting solution was stirred for another 4h until LCMS indicated all starting amine was consumed. Then the reaction solution was purified directly by Prep-HPLC (0.01% TFA) to yield PB100 ({4-[(2S)-2-[(2S)-2-[(2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-[6-(2,5-dioxo-2,5-dihydro-1 H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB100, 35 mg, 0.011 mmol, 65.64%)) as a white solid. ESI m/z: 621.9 (M/5+H)⁺, 777.0 (M/4+H)⁺, 1035.7 (M/3+H)⁺. Retention time 5.642 min (HPLC). ¹H NMR (400 MHz, DMSO-d6) δ 10.04 (s, 1H), 8.15-8.13 (m, 3H), 8.07 (d, J=9.2 Hz, 1H), 7.99-7.91 (m, 2H), 7.86-7.77 (m, 3H), 7.64 (d, J=8.8 Hz, 1H), 7.60 (d, J=8.4 Hz, 2H), 7.36 (d, J=8.4 Hz, 2H), 7.32 (s, 1H), 7.00 (s, 2H), 6.54 (s, 1H), 6.01 (t, J=5.6 Hz, 1H), 5.49-5.39 (m, 7H), 5.39-5.24 (m, 3H), 5.08 (s, 2H), 4.83-4.79 (m, 4H), 4.65-4.46 (m, 11H), 4.40-4.35 (m, 1H), 4.30-4.18 (m, 3H), 4.03-3.94 (m, 4H), 3.81-3.74 (m, 4H), 3.74-3.61 (m, 4H), 3.61-3.52 (m, 16H), 3.52-3.40 (m, 98H), 3.29-3.13 (m, 8H), 3.13-2.90 (m, 7H), 2.40-2.36 (m, 5H), 2.30 (t, J=6.4 Hz, 2H), 2.24-2.08 (m, 4H), 2.00-1.71 (m, 4H), 1.71-1.51 (m, 3H), 1.51-1.14 (m, 15H), 0.90-0.80 (m, 9H) ppm. Two protons signal of carboxyl group in TFA are revealed.

### Example 32: Preparation of a Drug-Linker Containing a PEG Unit and a Cleavable Linker Attached to MMAE (PB101 or “LD101”)

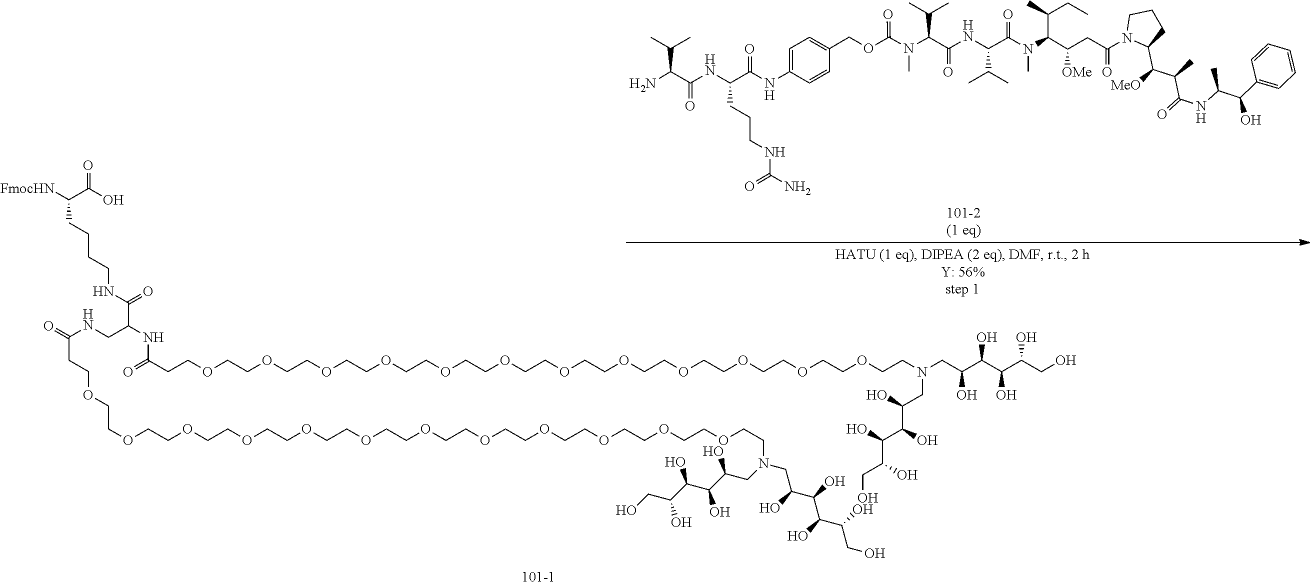

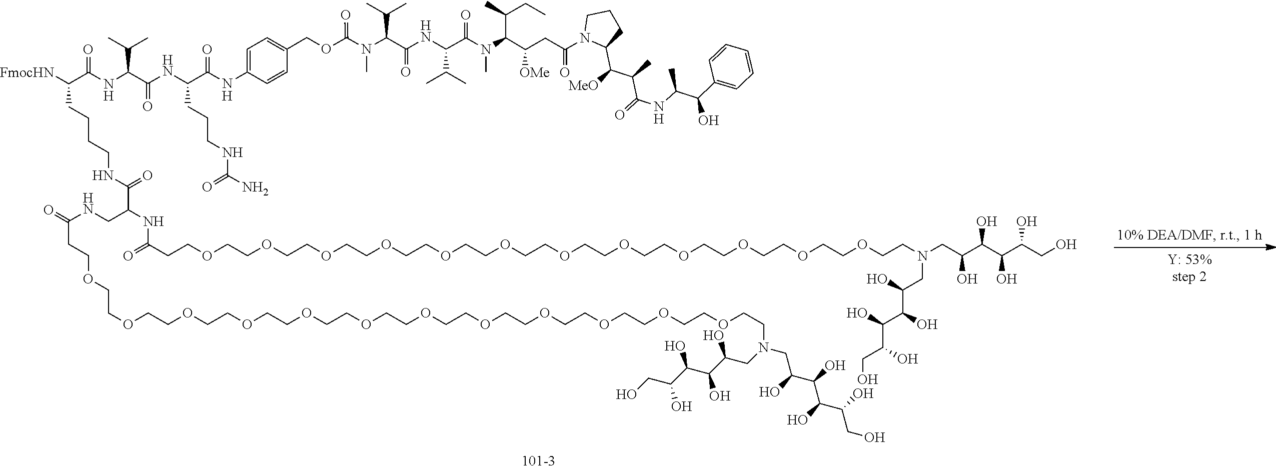

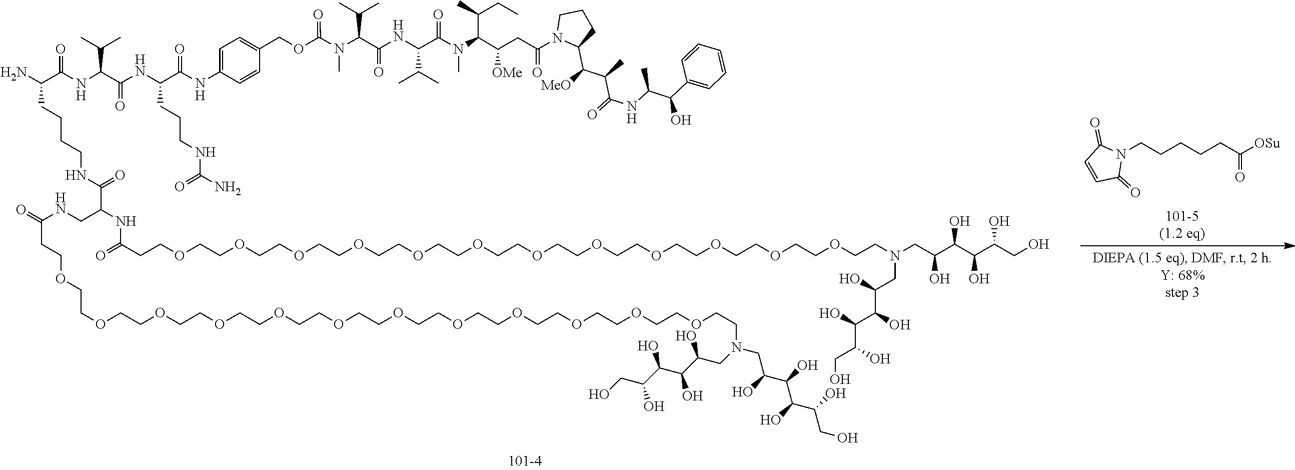

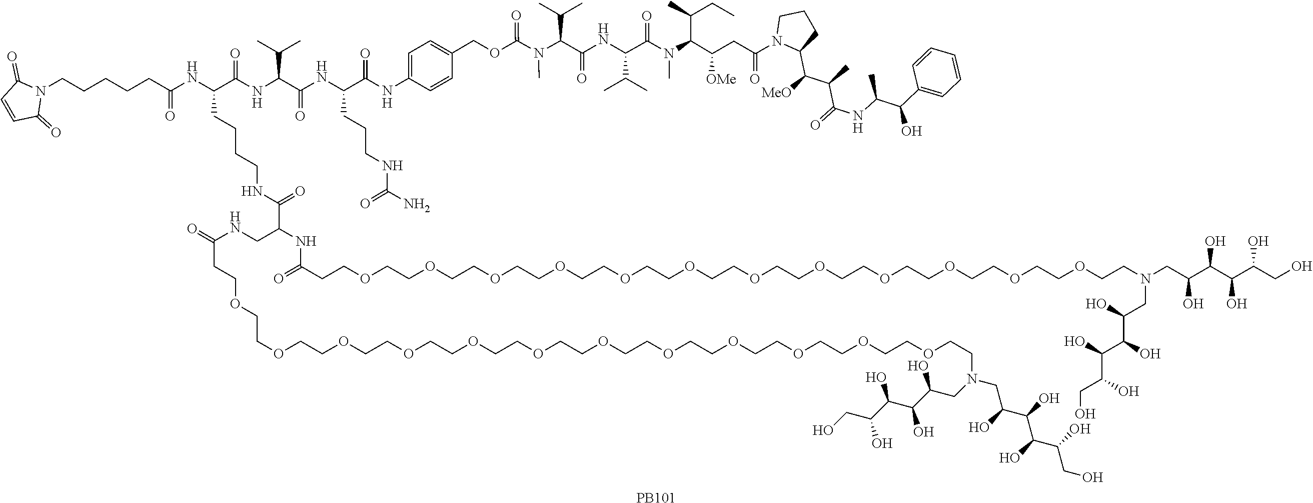

A Drug-Linker containing a PEG unit and a cleavable linker attached to MMAE (PB101 or LD101) was prepared as follows:

### Step 1

A solution of compound 101-1 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-carbamoylpentyl]carbamate (101-1, 250 mg, 0.108 mmol)), compound 100-2 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (101-2, 121.64 mg, 0.108 mmol)) and DIPEA (27.95 mg, 0.217 mmol) in anhydrous DMF (4 mL) was stirred at room temperature for 5 min, then a solution of HATU (41.19 mg, 0.108 mmol) in anhydrous DMF (4 mL) was added. The resulting solution was stirred for another 2 h until LCMS indicated a complete reaction. The reaction solution was purified directly by reverse phase flash chromatography (0.01% TFA) to give compound 101-3 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-{[(1S)-1-{[(1S)-4-(carbamoylamino)-1-({4-[({[(1S)-1-{[(1 S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (101-3, 210 mg, 0.061 mmol, 56.75%)) as a white solid. ESI m/z: 854.9 (M/4+H)⁺.

### Step 2

A solution of compound 101-3 ((9H-fluoren-9-yl)methyl N-[(1S)-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-1-{[(1S)-1-{[(1S)-4-(carbamoylamino)-1-({4-[({[(1S)-1-{[(1 S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (101-3, 200 mg, 0.059 mmol)) in DMF (3.6 mL) was stirred at room temperature and diethyl amine (0.4 mL, 3.883 mmol) was added. The resulting solution was stirred for 1 h to completion. Then diethyl amine was evaporated with a rotary evaporator, and the residue in DMF was purified by reverse phase flash chromatography (0.01% TFA) to yield the expected product 101-4 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (101-4, 100 mg, 0.031 mmol, 53.48%)) as a white solid. ESI m/z: 799.5(M/4+H)⁺.

### Step 3

A solution of compound 101-4 ({4-[(2S)-2-[(2S)-2-[(2S)-2-amino-6-{3-[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]-2-[(42S,43R,44R,45R)-42,43,44,45,46-pentahydroxy-40-[(3R,4S,5R)-3,4,5,6-tetrahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (101-4, 30 mg, 0.009 mmol)) and compound 101-5 (2,5-dioxopyrrolidin-1-yl 6-(2,5-dioxo-2,5-dihydro-1H-pyrrol-1-yl)hexanoate (101-5, 3.47 mg, 0.011 mmol)) in anhydrous DMF (1.5 mL) was stirred at room temperature, then DIPEA (1.74 mg, 0.013 mmol) was added. The resulting solution was stirred at room temperature for 2 h to achieve complete conversion (monitored by LCMS). Then the completed reaction solution was purified directly by Prep-HPLC (0.01% TFA) to get the desired fractions, which were lyophilized to yield PB101 ({4-[(2S)-2-[(2S)-2-[(2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-[6-(2,5-dioxo-2,5-dihydro-1 H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB101, 21 mg, 0.006 mmol, 66.02%)) as a white solid. ESI m/z: 847.5 (M/4+H)⁺, 1129.6 (M/3+H)⁺. Retention time 6.016 min (HPLC). ¹H NMR (400 MHz, DMSO-d6) δ 10.03 (s, 1H), 8.34-8.10 (m, 4H), 8.06-7.89 (m, 2H), 7.89-7.79 (m, 2H), 7.66-7.62 (m, 1H), 7.59-7.57 (m, 2H), 7.34-7.24 (m, 6H), 7.20-7.13 (m, 1H), 7.00 (s, 2H), 6.02-5.98 (m, 1H), 5.50-5.35 (m, 7H), 5.13-4.78 (m, 8H), 4.70-4.34 (m, 15H), 4.34-4.18 (m, 5H), 4.05-3.93 (m, 6H), 3.83-3.74 (m, 6H), 3.74-3.68 (m, 7H), 3.61-3.40 (m, 116H), 3.26-3.12 (m, 11H), 3.12-2.80 (m, 10H), 2.43-2.36 (m, 3H), 2.32-2.26 (m, 3H), 2.15-1.84 (m, 6H), 1.84-1.45 (m, 15H), 1.45-1.16 (m, 8H), 1.06-0.97 (m, 6H), 0.89-0.73 (m, 24H) ppm. One proton of carboxyl group in TFA was revealed.

### Example 33: Preparation of a Drug-Linker Containing a PEG Unit and a Cleavable Linker Attached to 6-Amino-9-{[4-(Aminomethyl)Phenyl]Methyl}-2-(2-Methoxyethoxy)-9H-Purin-8-Ol (PB102)

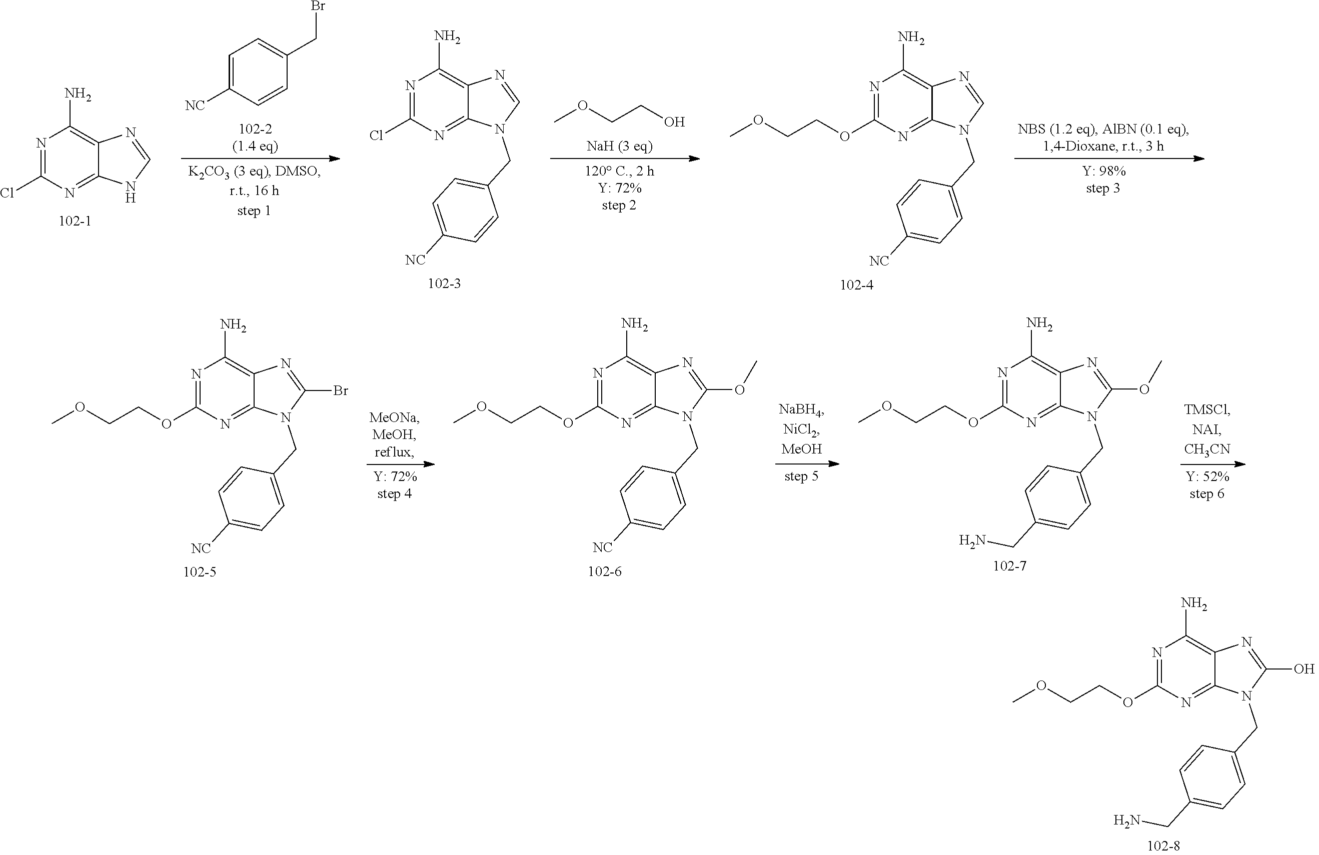

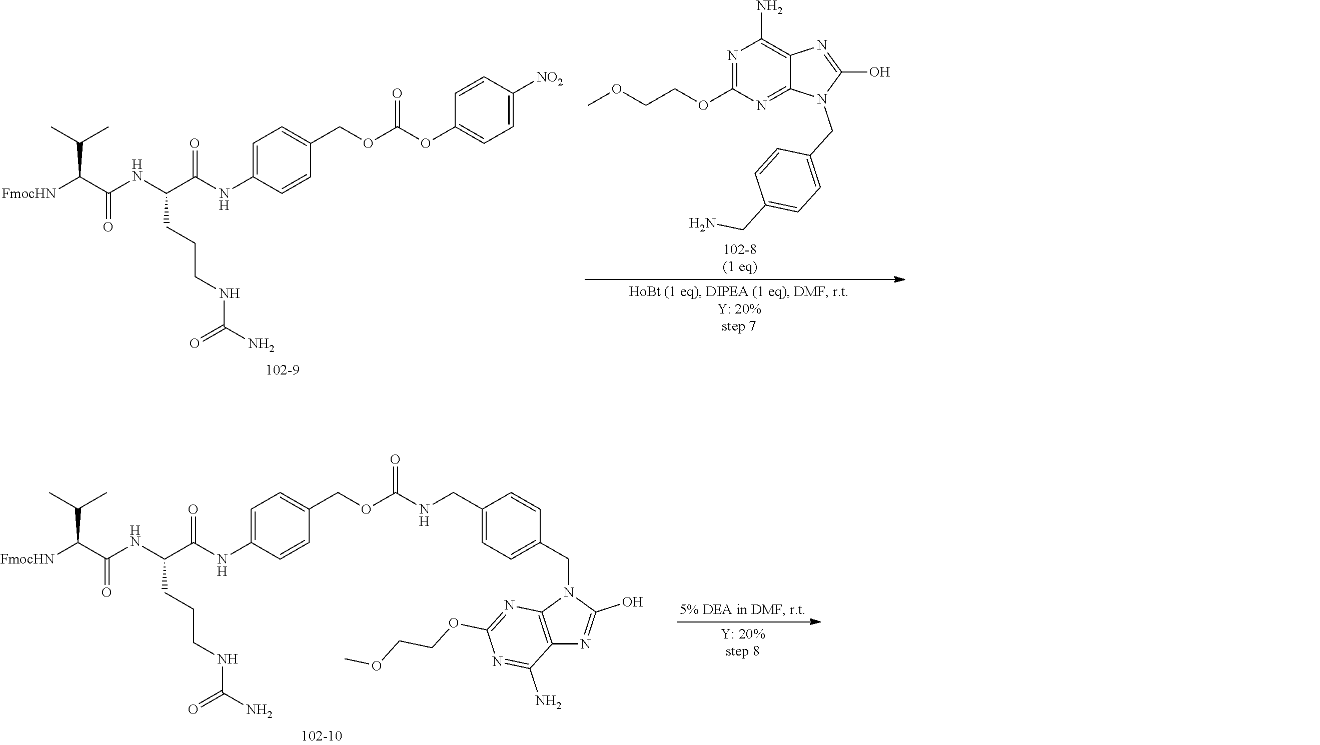

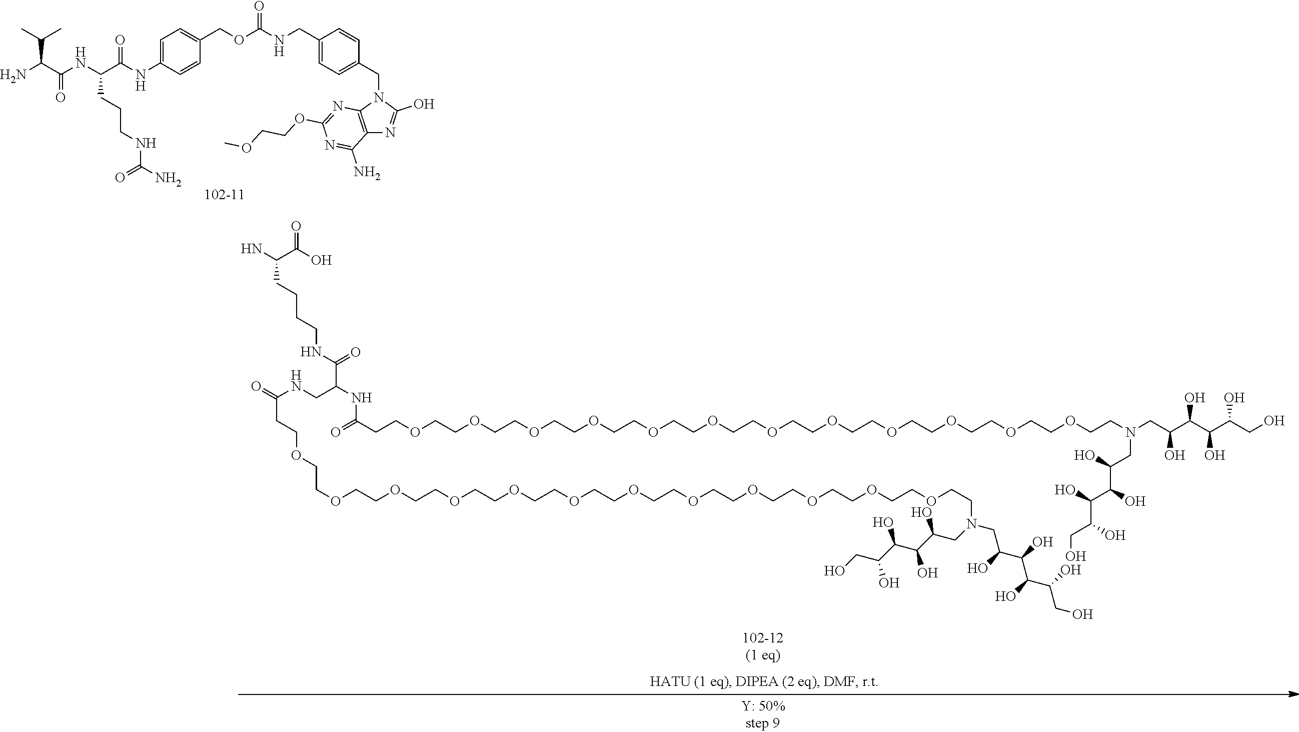 [SMILES: COCCOc1nc(N)c2nc(O)n(Cc3ccc(CNC(=O)OCc4ccc(NC(=O)[C@H](CCCNC(N)=O)NC(=O)[C@@H](N)C(C)C)cc4)cc3)c2n1 ; N[C@@H](CCCCNC(=O)C(CNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)O]

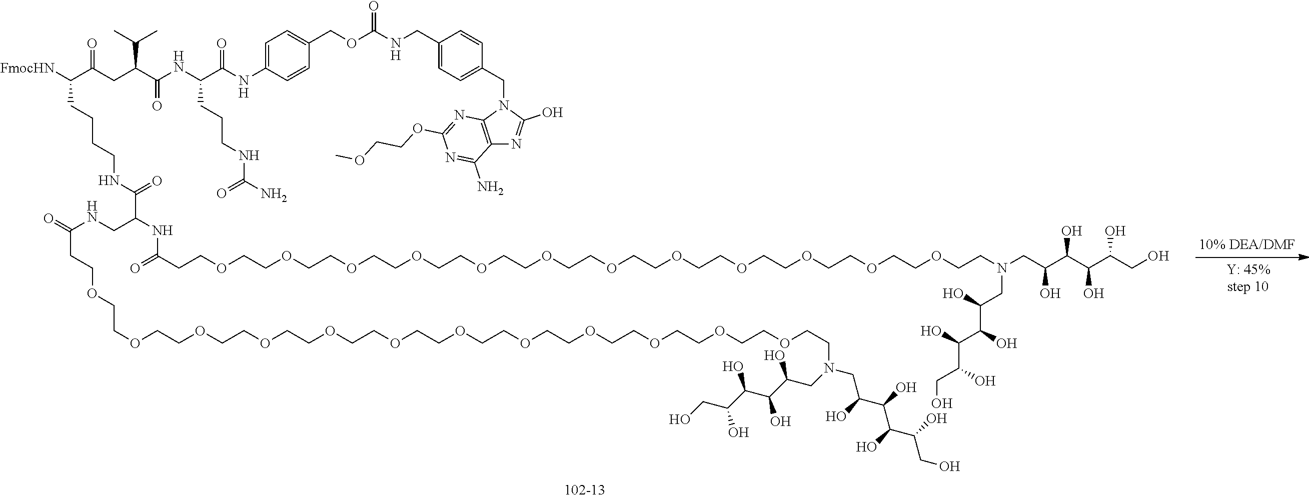

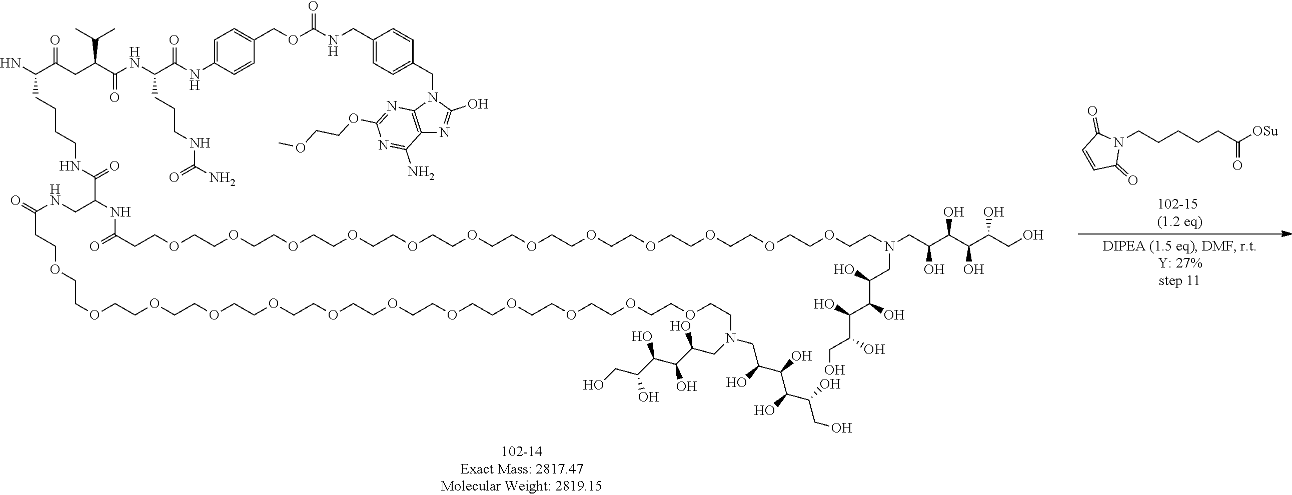

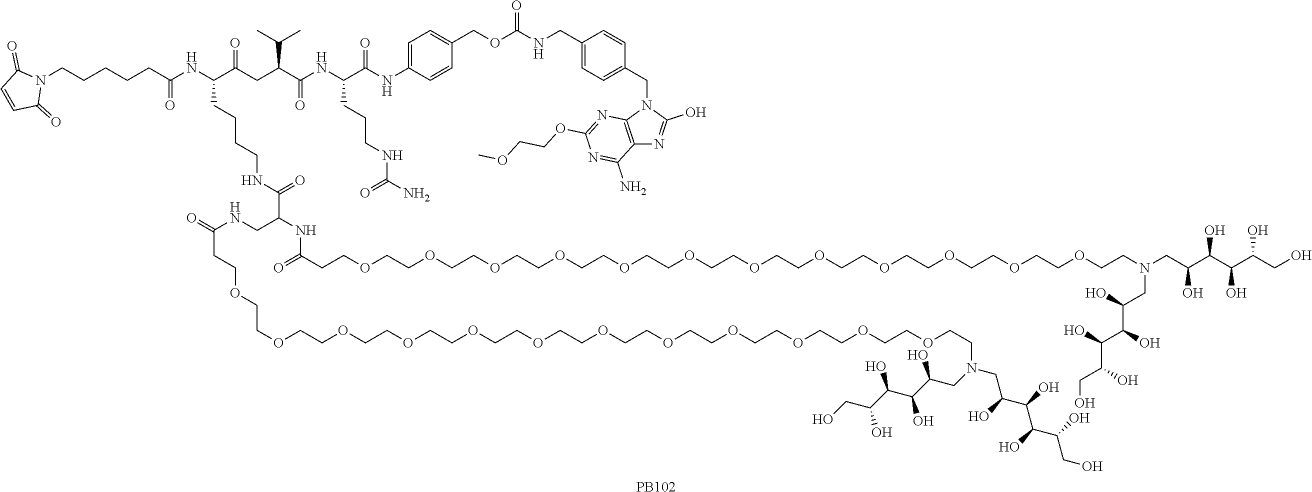

A Drug-Linker containing a PEG unit and a cleavable linker attached to 6-amino-9-{[4-(aminomethyl)phenyl]methyl}-2-(2-methoxyethoxy)-9H-purin-8-ol (PB102) was prepared as follows:

### Step 1

Compound 102-1 (2-chloro-9H-purin-6-amine) (1 eq) and K₂CO₃ (3 eq) is dissolved in DMSP and the reaction mixture is stirred. 4-(Bromomethyl) benzonitrile (1 0.4 eq) is added and the reaction mixture is stirred at room temperature for 16 hr. The reaction mixture is filtered to remove insoluble salts and poured into water. The aqueous phase is extracted with EtOAc. The combined organic phases are dried over Mg2SO4 and concentrated in vacuo to afford the product 102-3 (4-((6-amino-2-chloro-9H-purin-9-yl)methyl)benzonitrile (102-3)).

### Step 2

To a solution of NaH (1.24 g, 51.631 mmol) in 2-methoxyethan-1-ol (1.364 mL, 17.210 mmol) was added compound 102-3 (4-[(6-amino-2-chloro-9H-purin-9-yl)methyl]benzonitrile (102-3, 4.9 g, 17.210 mmol)). The mixture was stirred at 80° C. for 2h. The resulting solution was concentrated and purified by Biotage flash chromatography (silica gel, eluting with 0-10% MeOH in DCM) to afford the product 102-4 (4-{[6-amino-2-(2-methoxyethoxy)-9H-purin-9-yl]methyl}benzonitrile (102-4, 4.02 g, 12.394 mmol, 72.04%)) as a pale yellow solid. ESI m/z: 325.0(M+H)⁺.

### Step 3

To a solution of compound 102-4 (4-{[6-amino-2-(2-methoxyethoxy)-9H-purin-9-yl]methyl}benzonitrile (102-4, 4.02 g, 12.394 mmol)) in 1,4-dioxane (50 mL) was added sulfanylidene-λ\hat{ }4-boranimine (2.65 g, 14.873 mmol) and AIBN (0.183 mL, 1.239 mmol). The mixture was stirred at room temperature for 3h. The resulting solution was concentrated and purified by flash chromotography (silica gel, eluting with 0-10% MeOH in DCM) to afford the product 102-5 (4-{[6-amino-8-bromo-2-(2-methoxyethoxy)-9H-purin-9-yl]methyl}benzonitrile (102-5, 4.90 g, 12.152 mmol, 98%)) as a pale yellow solid. ESI m/z: 404.1(M+H)⁺.

### Step 4

To a solution of NaOMe (7.37 g, 136.395 mmol) in MeOH (50 mL) was added compound 102-5 (4-{[6-amino-8-bromo-2-(2-methoxyethoxy)-9H-purin-9-yl]methyl}benzonitrile (102-5, 5.5 g, 13.640 mmol)). The mixture was heated under reflux for 3h. The resulting solution was washed by brine and extracted with DCM (50 mL*3). The organic layer was dried over Na₂SO₄ and evaporated to afford the product 102-6 (4-{[6-amino-8-methoxy-2-(2-methoxyethoxy)-9H-purin-9-yl]methyl}benzonitrile (102-6, 3.5 g, 9.877 mmol, 72.46%). ESI m/z: 355.3(M+H)⁺).

### Step 5

To the solution of compound 102-6 (4-{[6-amino-8-methoxy-2-(2-methoxyethoxy)-9H-purin-9-yl]methyl}benzonitrile (102-6, 3.5 g, 9.877 mmol)) in MeOH (50 mL) was added NaBH₄ (2.258 mL, 69.137 mmol) and NiCl₂(H₂O)₆ (0.24 g, 0.988 mmol). The mixture was stirred at room temperature for 3h to completion (monitored by LCMS). The resulting solution was quenched by water and extracted with DCM (50 mL*3). The collected organic layer was dried over Na₂SO₄ and evaporated to afford the crude product 102-7 (9-{[4-(aminomethyl)phenyl]methyl}-8-methoxy-2-(2-methoxyethoxy)-9H-purin-6-amine (102-7, 3.6 g, 10.045 mmol, 101.70%)) as a pale yellow solid which was used directly in next step. ESI m/z: 359.3(M+H)⁺.

### Step 6

To a solution of compound 102-7 (9-{[4-(aminomethyl)phenyl]methyl}-8-methoxy-2-(2-methoxyethoxy)-9H-purin-6-amine (102-7, 3.5 g, 9.766 mmol)) in MeCN (20 mL) was added CISiMe₃ (1.06 g, 9.766 mmol) and Nal (0.400 mL, 9.766 mmol). The mixture was stirred at room temperature for 3h. The resulting solution was concentrated and purified by reverse phase flash chromatography (0.01% TFA) to afford the product 102-8 (6-amino-9-{[4-(aminomethyl)phenyl]methyl}-2-(2-methoxyethoxy)-9H-purin-8-ol (102-8, 1.75 g, 5.082 mmol, 52.08%)) a pale yellow solid. ESI m/z: 345.3(M+H)⁺, retention time 4.359 min (HPLC). ¹H NMR (400 MHz, DMSO-d6) δ 10.131 (s, 1H), 8.141 (s, 3H), 7.413-7.393 (d, J=8.0 Hz, 2H), 7.340-7.319 (d, J=8.4 Hz, 2H), 6.560 (s, 2H), 4.872 (s, 2H), 4.259-4.235 (t, J=4.8 Hz, 2H), 4.005-3.992 (d, J=5.2 Hz, 2H), 3.593-3.570 (t, J=4.6 Hz, 2H), 3.265 (s, 3H) ppm. One proton of carboxyl group in TFA was revealed.

### Step 7

To a solution of compound 102-8 (6-amino-9-{[4-(aminomethyl)phenyl]methyl}-2-(2-methoxyethoxy)-9H-purin-8-ol (102-8, 800 mg, 2.323 mmol)) in DMF (5 mL) was added compound 102-9 ({4-[(2S)-5-(carbamoylamino)-2-[(2S)-2-({[(9H-fluoren-9-yl)methoxy]carbonyl}amino)-3-methylbutanamido]pentanamido]phenyl}methyl 4-nitrophenyl carbonate (102-9, 1781.31 mg, 2.323 mmol), HOBt (313.89 mg, 2.323 mmol) and DIPEA (300.25 mg, 2.323 mmol). The mixture was stirred at room temperature for 2h. The resulting solution was purified by reverse phase flash chromatography (0.01% TFA) to afford the product 102-10 ((9H-fluoren-9-yl)methyl N-[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamate (102-10, 450 mg, 0.463 mmol, 19.93%)) as a plae yellow solid. ESI m/z: 972.5(M+H)⁺.

### Step 8

To a solution of compound 102-10 ((9H-fluoren-9-yl)methyl N-[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamate (102-10, 450 mg, 0.463 mmol)) in DMF (2 mL) was added diethylamine (0.1 mL, 1.276 mmol). The mixture was stirred at room temperature for 1h. Then the resulting solution was purified by reverse phase flash chromatography (0.01% TFA) to collect the desired fractions, which were lyophilized to afford impure product. The crude product was triturated in acetonitrile and filtered to give cake compound 102-11 ({4-[(2S)-2-[(2S)-2-amino-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-11, 70 mg, 0.093 mmol, 20.17%)) as a pale yellow solid. ESI m/z: 375.8(M/2+H)⁺.

### Step 9

To a solution of compound 102-11 ({4-[(2S)-2-[(2S)-2-amino-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-11, 58 mg, 0.077 mmol)) in DMF (4 mL) was added compound 102-12 ((2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-({[(9H-fluoren-9-yl)methoxy]carbonyl}amino)hexanoic acid (102-12, 178.73 mg, 0.077 mmol)), HATU (29.28 mg, 0.077 mmol) and DIPEA (9.95 mg, 0.077 mmol). The resulting solution was stirred at room temperature for 1h, then was purified by reverse phase flash chromatography (0.01% TFA) to afford the product fractions, which were freeze-dried to yield compound 102-13 ((9H-fluoren-9-yl)methyl N-[(1S)-1-{[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamoyl}-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}pentyl]carbamate (102-13, 120 mg, 0.040 mmol, 51.30%)) as a white solid. ESI m/z: 761.3(M/4+H)⁺.

### Step 10

To a solution of compound 102-13 ((9H-fluoren-9-yl)methyl N-[(1S)-1-{[(1S)-1-{[(1S)-1-({4-[({[(4-{[6-amino-8-hydroxy-2-(2-methoxyethoxy)-9H-purin-9-yl]methyl}phenyl)methyl]carbamoyl}oxy)methyl]phenyl}carbamoyl)-4-(carbamoylamino)butyl]carbamoyl}-2-methylpropyl]carbamoyl}-5-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}pentyl]carbamate (102-13, 120 mg, 0.039 mmol)) in DMF (2 mL) was added diethylamine (0.1 mL, 1.276 mmol). The mixture was stirred at room temperature for 1h. The resulting solution was purified by reverse phase separation (0.01% TFA) to afford the product 102-14 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-14, 50 mg, 0.018 mmol, 44.95%)) as a white solid. ESI m/z: 706.0(M/4+H)⁺.

### Step 11

To the solution of compound 102-14 ({4-[(2S)-2-[(2S)-2-[(2S)-2-amino-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}hexanamido]-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (102-14, 50 mg, 0.018 mmol)) in DMF (1 mL) was added compound 102-15 (2,5-dioxopyrrolidin-1-yl 6-(2,5-dioxo-2,5-dihydro-1H-pyrrol-1-yl)hexanoate (102-15, 6.56 mg, 0.021 mmol)) and DIPEA (3.44 mg, 0.027 mmol). The solution was stirred at room temperature for 2 h until all starting amine was consumed. Then the resulting solution was purified directly by Prep-HPLC (0.01% TFA) to afford the product fractions, which were lyophilized to yield PB102 ({4-[(2S)-2-[(2S)-2-[(2S)-6-{2,3-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]propanamido}-2-[6-(2,5-dioxo-2,5-dihydro-1 H-pyrrol-1-yl)hexanamido]hexanamido]-3-methylbutanamido]-5-(carbamoylamino)pentanamido]phenyl}methyl N-[(4-{[6-amino-8-hydroxy-2-(2-methoxyethoxy)-9H-purin-9-yl]methyl}phenyl)methyl]carbamate (PB102, 20 mg, 0.007 mmol, 37.43%)) as a white solid. ESI m/z: 754.5 (M/4+H)⁺, 1005.1 (M/3+H)⁺, retention time 5.190 min (HPLC). ¹H NMR (400 MHz, DMSO-d6) δ 10.03 (s, 1H), 10.01 (s, 1H), 8.15-8.13 (m, 3H), 7.97 (d, J=8.0 Hz, 1H), 7.94 (d, J=4.0 Hz, 1H), 7.87-7.85 (m, 2H), 7.81-7.75 (m, 1H) 7.66 (d, J=12.0 Hz, 1H), 7.58 (d, J=4.0 Hz, 2H), 7.29-7.19 (m, 6H), 7.00 (s, 2H), 6.50 (s, 2H), 6.01 (t, J=5.6 Hz, 1H), 5.44 (brs, 4H), 4.95 (brs, 2H), 4.82 (brs, 2H), 4.57-4.39 (m, 12H), 4.26-4.14 (m, 8H), 4.05-3.95 (m, 4H), 3.80-3.76 (m, 4H), 3.68-3.61 (m, 4H), 3.58-3.56 (m, 18H), 3.52-3.36 (m, 98H), 3.35-3.29 (m, 12H), 3.26-3.17 (m, 7H), 3.01-2.92 (m, 4H), 2.68-2.66 (m, 1H), 2.39 (t, J=6 Hz, 2H), 2.33-2.29 (m, 2H), 2.20-2.10 (m, 2H), 1.99-1.95 (m, 1H), 1.74-1.55 (m, 3H), 1.46-1.31 (m, 8H), 1.25-1.10 (m, 4H), 0.85-0.81 (m, 6H) ppm. Two protons of carboxyl group in TFA were revealed.

### Example 34: Preparation of a Drug-Linker Containing a PEG Unit and a Cleavable Linker Attached to Exatecan (PB103)

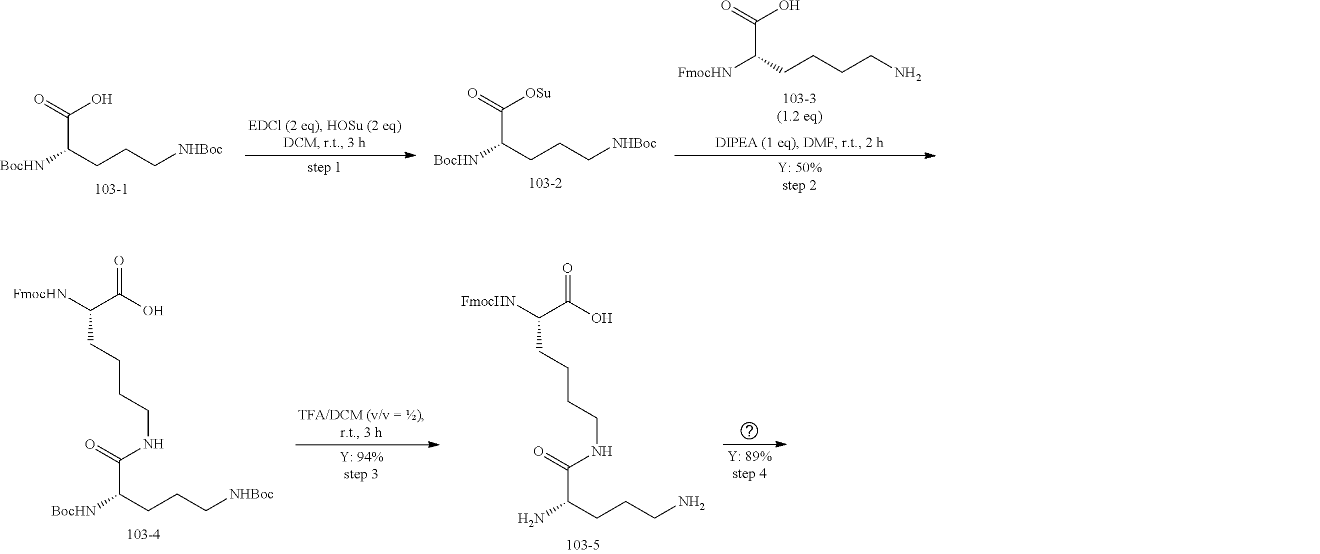

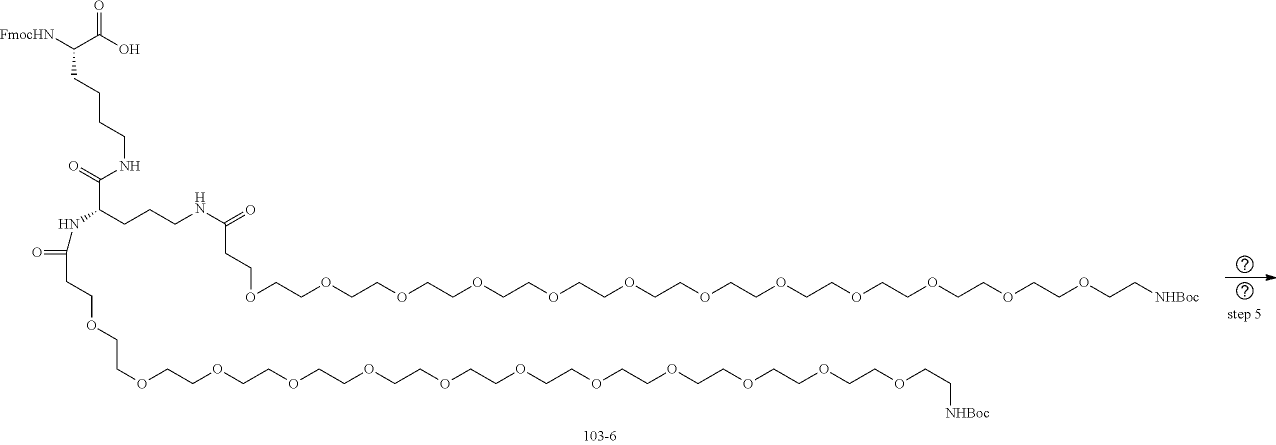

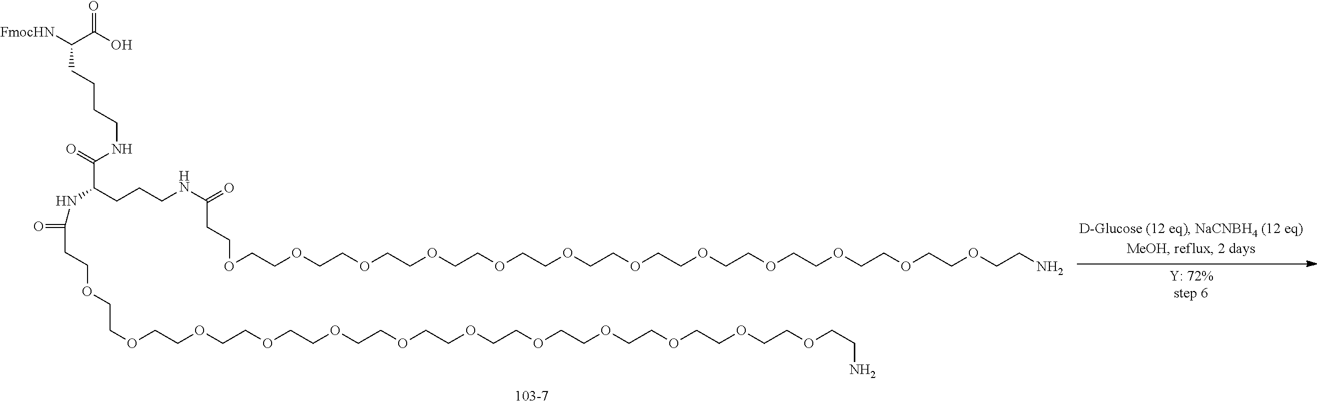

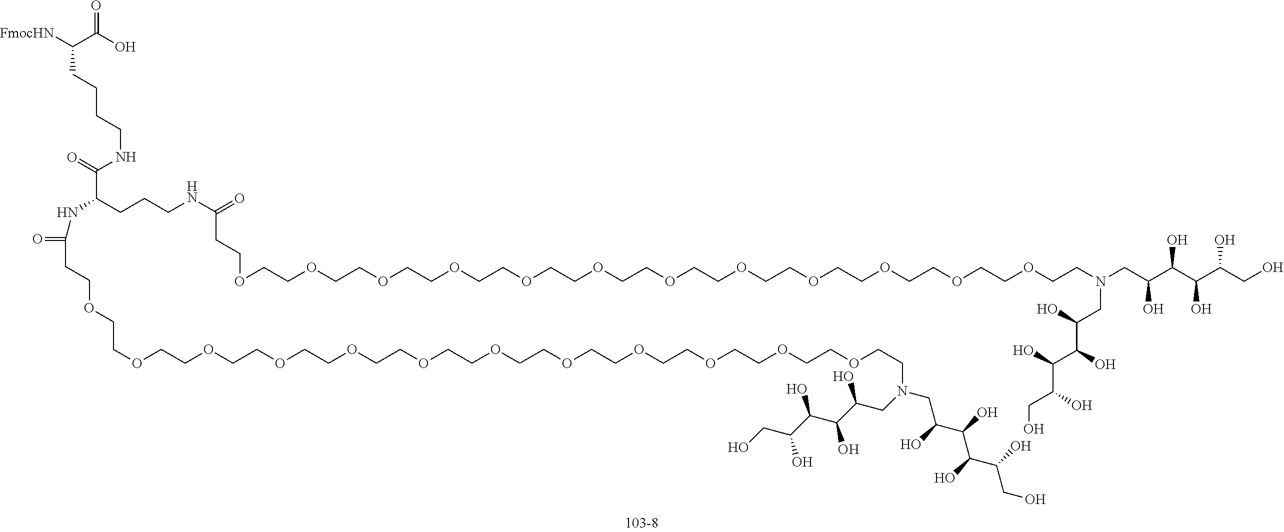

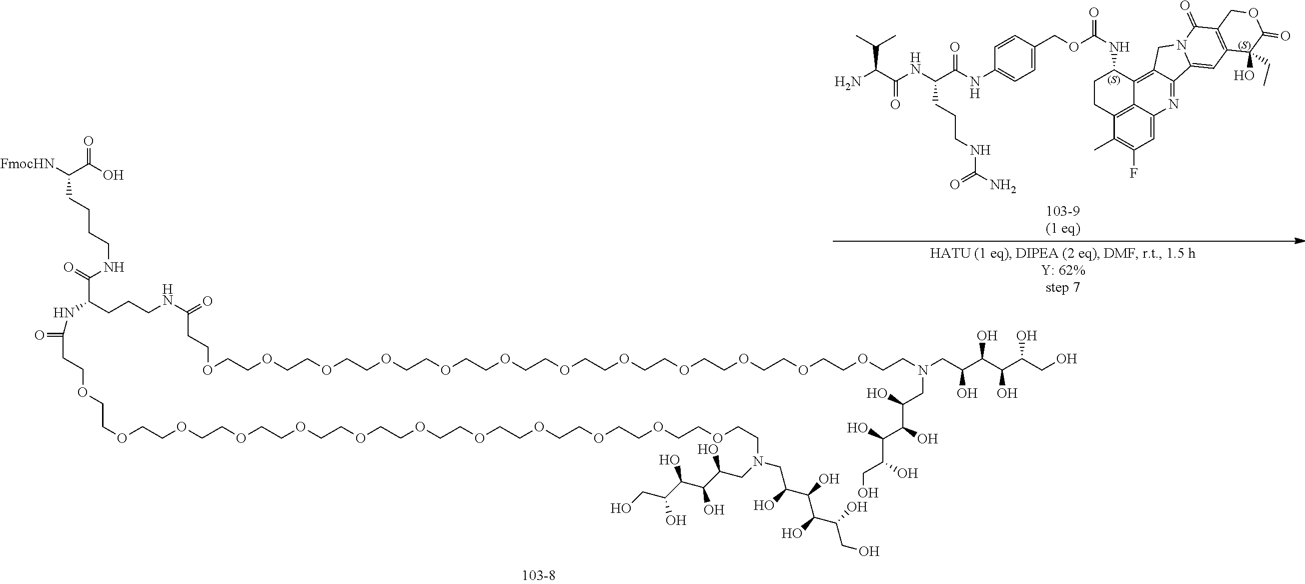

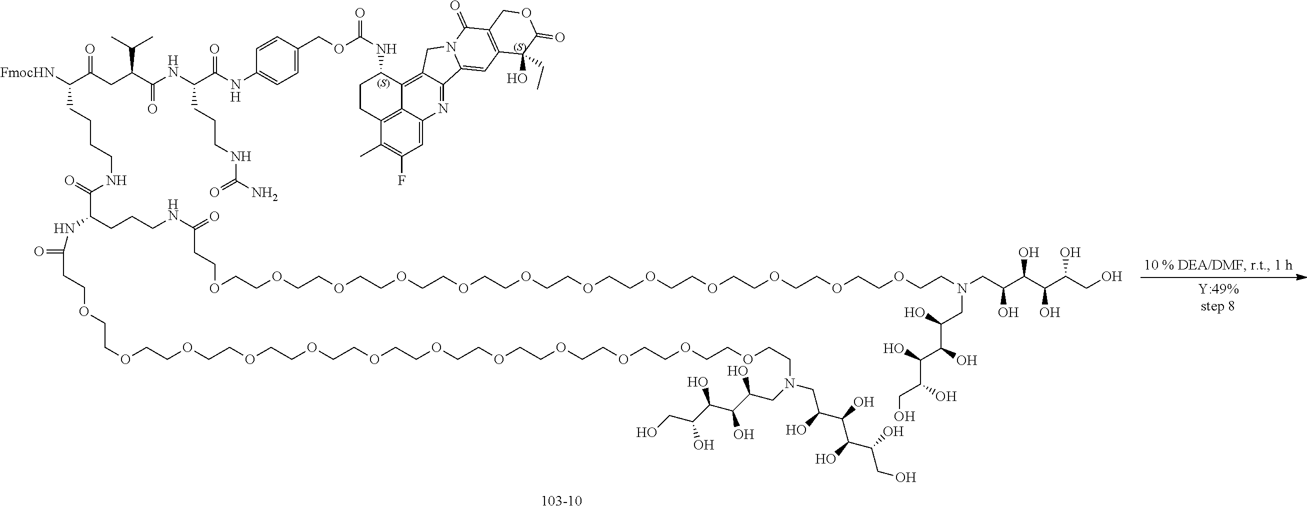

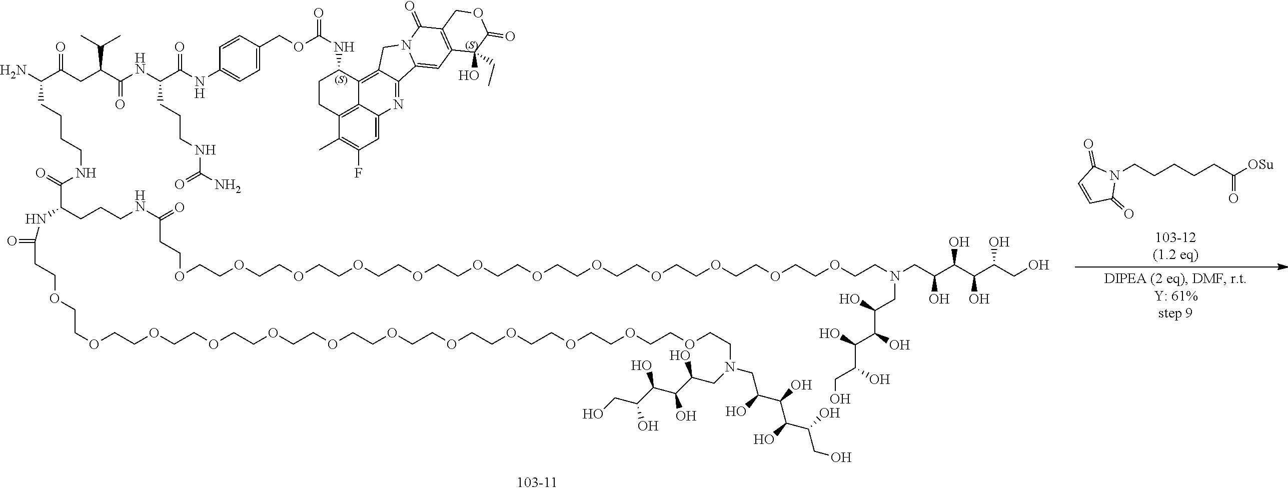

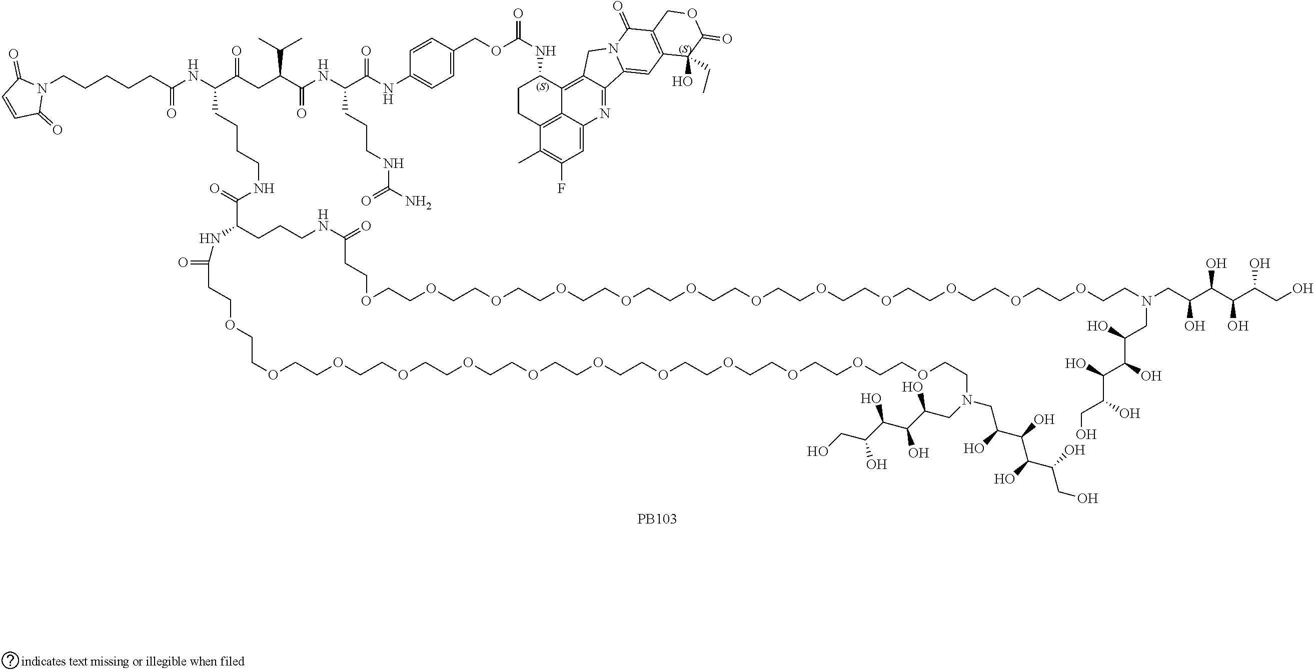

A Drug-Linker containing a PEG unit and a cleavable linker attached to exatecan (PB103) was prepared as follows:

### Step 1

To a solution of compound 103-1 ((2S)-2,5-bis({[(tert-butoxy)carbonyl]amino})pentanoic acid (103-1, 4.5 g, 13.538 mmol)) in DCM (20 mL) was added HOSu (3.12 g, 27.076 mmol) and EDCl (5.19 g, 27.076 mmol). The mixture was stirred at room temperature for 3h. The resulting solution was washed by water and extracted by DCM. The organic layer was dried over Na₂SO₄ and evaporated to afford the crude product 103-2 (2,5-dioxopyrrolidin-1-yl (S)-2,5-bis((tert-butoxycarbonyl)amino)pentanoate (103-2, 6.0 g, 13.971 mmol, 103.27%)). ESI m/z: 452.3(M+Na)⁺.

### Step 2

To a solution of compound 103-2 (2,5-dioxopyrrolidin-1-yl (S)-2,5-bis((tert-butoxycarbonyl)amino)pentanoate (103-2, 5.81 g, 13.528 mmol)) in DMF (20 mL) was added compound 103-3 ((2S)-6-amino-2-({[(9H-fluoren-9-yl)methoxy]carbonyl}amino)hexanoic acid (103-3, 5.98 g, 16.234 mmol)) and DIPEA (1.75 g, 13.528 mmol). The mixture was stirred at room temperature for 2h. The resulting solution was purified by reverse phase flash chromatography (0.01% TFA) to afford the product 103-4 ((2S)-6-[(2S)-2,5-bis({[(tert-butoxy)carbonyl]amino})pentanamido]-2-({[(9H-fluoren-9-yl)methoxy]carbonyl}amino)hexanoic acid (103-4, 8.5 g, 12.448 mmol, 91.99%)) as white solid. ESI m/z: 683.5(M+H)⁺.

### Step 3

To a solution of compound 103-4 ((2S)-6-[(2S)-2,5-bis({[(tert-butoxy)carbonyl]amino})pentanamido]-2-({[(9H-fluoren-9-yl)methoxy]carbonyl}amino)hexanoic acid (103-4, 8.5 g, 12.448 mmol)) in DCM (20 mL) was added TFA (10 mL, 134.626 mmol). The mixture was stirred at room temperature for 3h. The resulting solution was evaporated and purified by reverse phase flash chromatography (0.01% TFA) to afford the product 103-5 ((2S)-6-[(2S)-2,5-diaminopentanamido]-2-({[(9H-fluoren-9-yl)methoxy]carbonyl}amino)hexanoic acid (103-5, 5.7 g, 11.812 mmol, 94.84%)) as white solid. ESI m/z: 483.4(M+H)⁺.

### Step 4

To a solution of 2,5-dioxopyrrolidin-1-yl 2,2-dimethyl-4-oxo-3,8,11,14,17,20,23,26,29,32,35,38,41-tridecaoxa-5-azatetratetracontan-44-oate (5.68 g, 6.970 mmol) in DMF (15 mL) was added DIPEA (0.90 g, 6.970 mmol) and compound 103-5 ((2S)-6-[(2S)-2,5-diaminopentanamido]-2-({[(9H-fluoren-9-yl)methoxy]carbonyl}amino)hexanoic acid (103-5, 1.68 g, 3.485 mmol)). The mixture was stirred at room temperature for 1h. The resulting solution was purified by reverse phase flash chromatography (0.01% TFA) to afford the product 103-6 ((2S)-6-[(2S)-2,5-bis(1-{[(tert-butoxy)carbonyl]amino}-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-6, 5.9 g, 3.135 mmol, 89.94%)) as transparent oil. ESI m/z: 561.2((M-200)/3+H)⁺.

### Step 5

To a solution of compound 103-6 ((2S)-6-[(2S)-2,5-bis(1-{[(tert-butoxy)carbonyl]amino}-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-6, 5.9 g, 3.135 mmol)) in DCM (10 mL) was added TFA (5 mL, 67.313 mmol). The mixture was stirred at room temperature overnight. The resulting solution was concentrated and purified by reverse phase separation (C18 column, eluting with 0-40% acetonitrile in water with TFA) to afford the product 103-7 ((2S)-6-[(2S)-2,5-bis(1-amino-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-7, 4.5 g, 2.675 mmol, 85.39%)) as transparent oil. ESI m/z: 561.6(M/3+H)⁺.

### Step 6

To a solution of compound 103-7 ((2S)-6-[(2S)-2,5-bis(1-amino-3,6,9,12,15,18,21,24,27,30,33,36-dodecaoxanonatriacontan-39-amido)pentanamido]-2-({[(9H-fluoren-9-yl)methoxy]carbonyl}amino)hexanoic acid (103-7, 4.5 g, 2.675 mmol)) in MeOH (30 mL) was added D-glucose (5.78 g, 32.104 mmol) and NaBH₃CN (1.949 mL, 32.104 mmol). The mixture was stirred at 60° C. over the weekend. The resulting solution was purified by reverse phase flash chromatography (0.01% TFA) to afford the product 103-8 ((2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-({[(9H-fluoren-9-yl)methoxy]carbonyl}amino)hexanoic acid (103-8, 4.53 g, 1.937 mmol, 72.36%)) as a white gel. ESI m/z: 780.4(M/3+H)⁺.

### Step 7

To a solution of compound 103-8 ((2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-({[(9H-fluoren-9-yl)methoxy]carbonyl}amino)hexanoic acid (103-8, 330 mg, 0.141 mmol)) in DMF (5 mL) was added compound 103-9 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamate (103-9, 118.66 mg, 0.141 mmol)), HATU (53.65 mg, 0.141 mmol) and DIPEA (36.47 mg, 0.282 mmol). The mixture was stirred at room temperature for 1.5h. The resulting solution was adjusted to pH 6 and purified by reverse phase flash chromatography (0.01% TFA) to afford the product 103-10 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1S)-1-{[(1 S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (103-10, 273 mg, 0.086 mmol, 61.19%)) as a pale yellow solid. ESI m/z: 790.9(M/4+H)⁺.

### Step 8

To the solution of compound 103-10 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (103-10, 273 mg, 0.086 mmol)) in DMF (2.5 mL) was added diethyl amine (0.5 mL, 6.378 mmol). The mixture was stirred at room temperature for 1 h. The resulting solution was purified by reverse phase flash chromatography (0.01% TFA) to afford the product 103-11 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (103-11, 123 mg, 0.042 mmol, 48.46%)) as a pale yellow solid. ESI m/z: 735.7(M/4+H)⁺, 980.7(M/3+H)⁺.

### Step 9

To the solution of compound 103-11 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (103-11, 123 mg, 0.042 mmol)) in DMF (3 mL) was added 2,5-dioxopyrrolidin-1-yl 6-(2,5-dioxo-2,5-dihydro-1H-pyrrol-1-yl)hexanoate (103-12, 19.35 mg, 0.063 mmol)) and DIPEA (8.11 mg, 0.063 mmol). The mixture was stirred at room temperature for 1h. The resulting solution was adjusted to pH 6 and purified by Prep-HPLC (0.01% TFA) to afford the product PB103 ({4-[(2S)-2-[(2S)-2-[(2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-[6-(2,5-dioxo-2,5-dihydro-1 H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB103, 80 mg, 0.026 mmol, 61.03%)) as a pale yellow solid. ESI m/z: 627.7(M/5+H)⁺, 784.0(M/4+H)⁺, 1044.9(M/3+H)⁺, retention time 5.614 min (HPLC). ¹H NMR (400 MHz, DMSO-d6) δ 10.05 (s, 1H), 8.16-8.14 (m, 3H), 8.09-8.07 (d, J=8.8 Hz, 1H), 7.99-7.98 (d, J=7.2 Hz, 2H), 7.86-7.83 (m, 2H), 7.80-7.77 (d, J=10.8 Hz, 1H), 7.67-7.65 (d, J=8.0 Hz, 1H), 7.61-7.59 (d, J=8.4 Hz, 2H), 7.37-7.35 (d, J=8.8 Hz, 2H), 7.311 (s, 1H), 7.00 (s, 2H), 6.55 (s, 1H), 6.03-6.01 (t, J=5.2 Hz, 1H), 5.45 (brs, 8H), 5.29-5.23 (m, 3H), 5.07 (s, 2H), 4.82 (brs, 4H), 4.62-4.37 (m,12H), 4.24-4.16 (m,3H), 3.98 (brs, 4H), 3.78-3.77 (m, 4H), 3.677 (brs, 4H), 3.61-3.54 (m, 18H), 3.50-3.43 (m, 98H), 3.29-3.23 (m, 7H), 3.17-2.94 (m, 6H), 2.43-2.41 (m, 5H), 2.30-2.26 (m, 3H), 2.24-2.08 (m, 4H), 2.00-1.83 (m, 3H), 1.66-1.59 (m, 4H), 1.49-1.42 (m, 7H), 1.36-1.45 (m, 6H), 1.29-1.14 (m, 4H), 0.89-0.80 (m, 9H) ppm.

### Example 35: Preparation of a Drug-Linker Containing a PEG Unit and a Cleavable Linker Attached to MMAE (PB104)

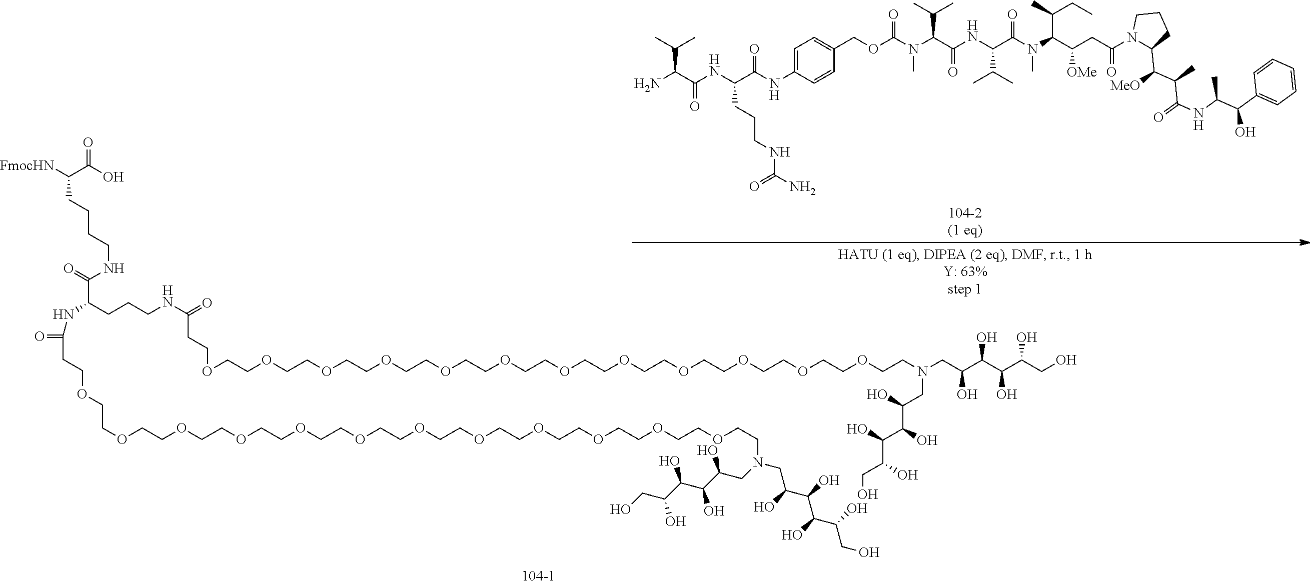

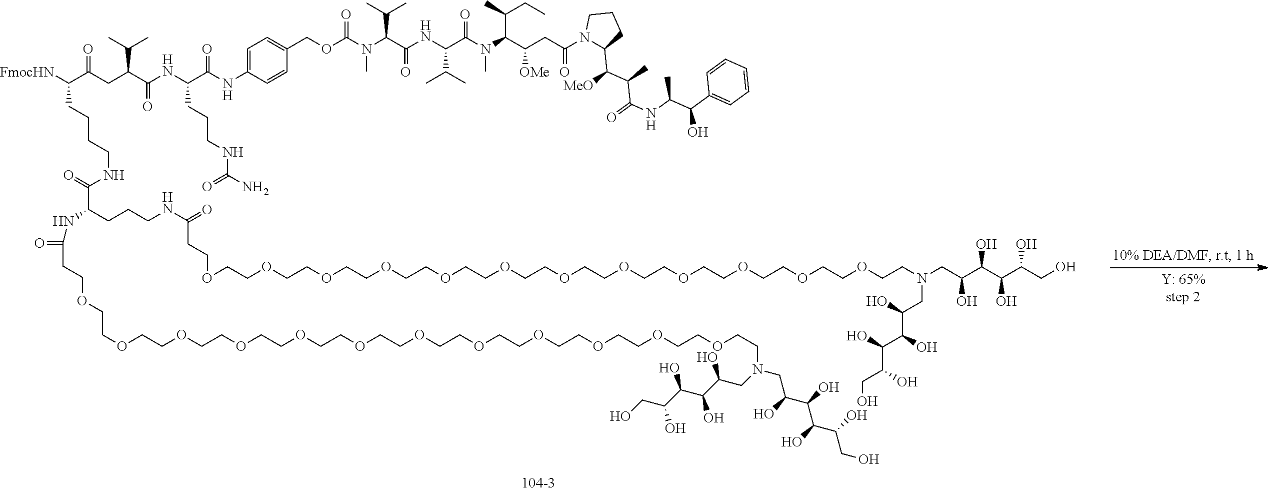

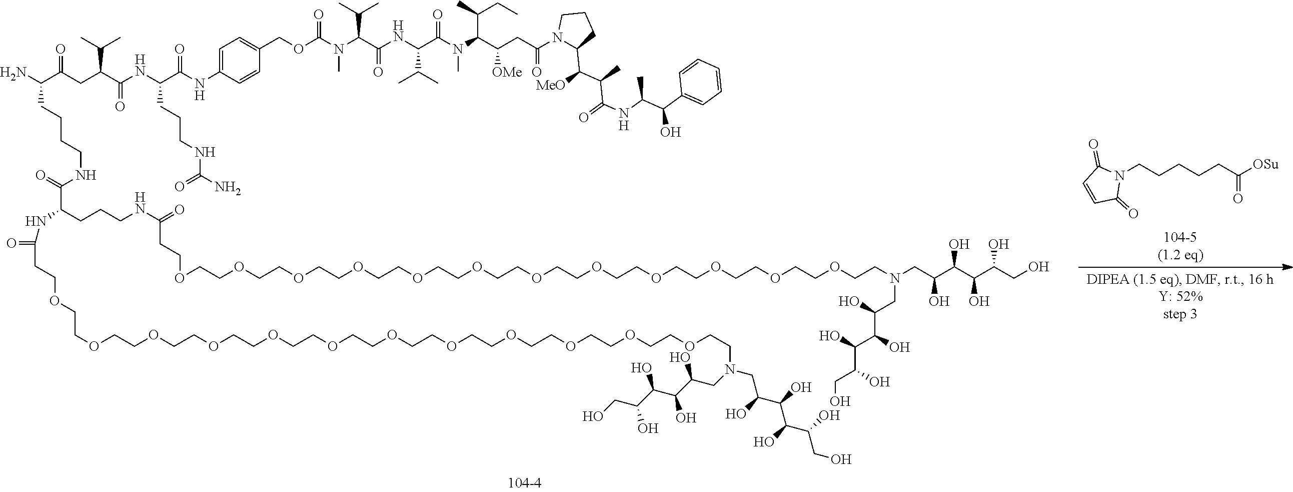

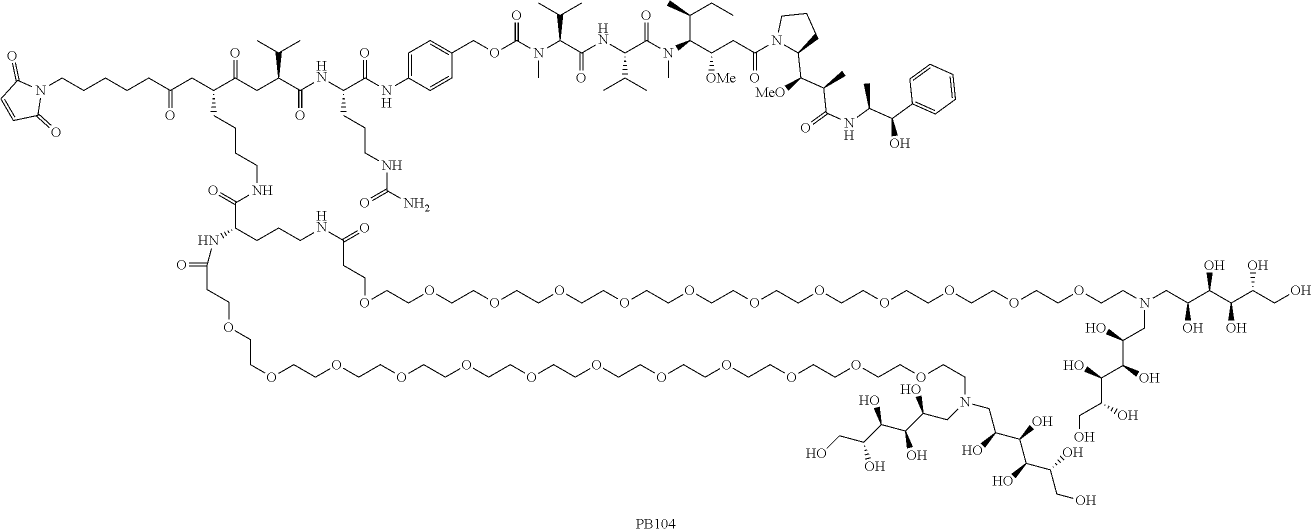

A Drug-Linker containing a PEG unit and a cleavable linker attached to MMAE (PB104) was prepared as follows:

### Step 1

A solution of compound 104-1 ((2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31, 34,37-dodecaoxa-40-azahexatetracontanamido]pentanamido]-2-({[(9H-fluoren-9-yl)methoxy]carbonyl}amino)hexanoic acid (104-1, 300 mg, 0.128 mmol)), compound 104-2 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (104-2, 144.10 mg, 0.128 mmol)) and DIPEA (33.02 mg, 0.256 mmol) in anhydrous DMF (3 mL) was stirred at room temperature for 5 min, then a solution of HATU (48.79 mg, 0.128 mmol) in anhydrous DMF (1 mL) was added slowly. The resulting solution was stirred for another1 h until LCMS indicated complete consumption of the starting amine. Then the reaction solution was purified by reverse phase flash chromatography (0.01% TFA) to yield compound 104-3 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1 S)-1-{[(1S)-4-(carbamoylamino)-1-({4-[({[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (104-3, 280 mg, 0.081 mmol, 63.51%)) as a white solid. ESI m/z: 671.5 ((M-717-26-18)/4+H)⁺, 862.0 (M/4+H)⁺.

### Step 2

A solution of compound 104-3 ((9H-fluoren-9-yl)methyl N-[(1S)-5-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-1-{[(1S)-1-{[(1S)-4-(carbamoylamino)-1-({4-[({[(1S)-1-{[(1 S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (104-3, 260 mg, 0.075 mmol)) in DMF (3.6 mL) was stirred at room temperature and diethyl amine (0.4 mL, 3.883 mmol) was added. The resulting solution was stirred for another 1 h until LCMS showed that the reaction was completed. Volatiles (especially diethyl amine) were evaporated off under vacuo, and the residue was purified by reverse phase column chromatography (0.01% TFA) to yield product 104-4 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (104-4, 160 mg, 0.050 mmol, 65.78%)) as a white solid. ESI m/z=615.8 ((M-717-26-18)/4+H)⁺, 806.3 (M/4+H)⁺. ¹H NMR (400 MHz, DMSO-d6) δ 10.10 (s, 1H), 8.43 (d, J=8.8 Hz, 1H), 8.35-8.33 (m, 1.5H), 8.17-8.09 (m, 5H), 8.03 (d, J=7.6 Hz, 1H), 7.97-7.78 (m, 3H), 7.66 (d, J=9.2 Hz, 0.5H), 7.59-7.57 (m, 2H), 7.49-7.40 (m, 1H), 7.32-7.24 ((m, 6H), 7.20-7.13 ((m, 1H), 6.04 (d, J=4.2 Hz, 1H), 5.52-5.36 ((m, 7H), 5.11-4.94 ((m, 3H), 4.89-4.69 ((m, 5H), 4.69-4.39 ((m, 16H), 4.30-4.17 (m, 4H), 4.04-3.93 (m, 7H), 3.88-3.81 (m, 2H), 3.79-3.76 (m, 5H), 3.72-3.62 (m, 6H), 3.62-3.49 (m, 88H), 3.43-3.25 (m, 20H), 3.25-3.06 (m, 12H), 3.06-2.83 (m, 11H), 2.40-2.38 (m, 2H), 2.30-2.22 (m, 3H), 2.14-1.92 (m, 4H), 1.84-1.62 (m, 6H), 1.62-1.21 (m, 16H), 1.05-0.97 (m, 6H), 0.97-0.75 (m, 26H) ppm. Three protons of carboxyl groups in TFA were revealed.

### Step 3

A solution of compound 104-4 ({4-[(2S)-2-[(2S)-2-[(2S)-2-amino-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (104-4, 100 mg, 0.029 mmol) and DIPEA (5.65 mg, 0.044 mmol)) in anhydrous DMF (1.5 mL) was stirred at room temperature for 5 min, then a solution of compound 104-5 (2,5-dioxopyrrolidin-1-yl 6-(2,5-dioxo-2,5-dihydro-1H-pyrrol-1-yl)hexanoate (104-5, 10.80 mg, 0.035 mmol)) in anhydrous DMF (0.5 mL) was added dropwise by syringe over 5 min. After addition, the resulting solution was stirred at room temperature for overnight for convenience, and LCMS indicated complete reaction in the morning. The resulting solution was purified directly by Prep-HPLC (0.01% TFA) to yield PB104 ({4-[(2S)-2-[(2S)-2-[(2S)-6-[(2S)-2,5-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]pentanamido]-2-[6-(2,5-dioxo-2,5-dihydro-1 H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB104, 55 mg, 0.016 mmol, 55.11%)) as a white solid. ESI m/z: 854.5 (M/4+H)⁺; retention time 5.614 min (HPLC). ¹H NMR (400 MHz, DMSO-d6) δ 10.03 (s, 1H), 8.34-8.10 (m, 4H), 8.00-7.97 (m, 2H), 7.93-7.83 (m, 2.5H), 7.67-7.64 (m, 1.5H), 7.58 (d, J=8.0 Hz, 2H), 7.34-7.25 (m, 6H), 7.19-7.13 (m, 1H), 7.00 ((s, 2H), 6.02-5.99 ((m, 1H), 5.52-5.38 ((m, 7H), 5.14-4.94 ((m, 3H), 4.94-4.18 ((m, 25H), 4.04-3.93 (m, 7H), 3.79-3.77 (m, 5H), 3.69-3.66 (m, 5H), 3.62-3.56 (m, 20H), 3.52-3.47 (m, 88H), 3.36-3.12 (m, 15H), 3.03-2.83 (m, 13H), 2.43-2.37 (m, 3H), 2.30-2.27 (m, 3H), 2.15-2.06 (m, 4H), 2.01-1.94 (m, 2H), 1.81-1.50 (m, 8H), 1.50-1.17 (m, 20H), 1.05-0.97 (m, 6H), 0.89-0.75 (m, 26H) ppm. Two protons of carboxyl groups in TFA were revealed.

### Example 36: Preparation of a Drug-Linker Containing a PEG Unit and a Cleavable Linker Attached to MMAE (PB105)

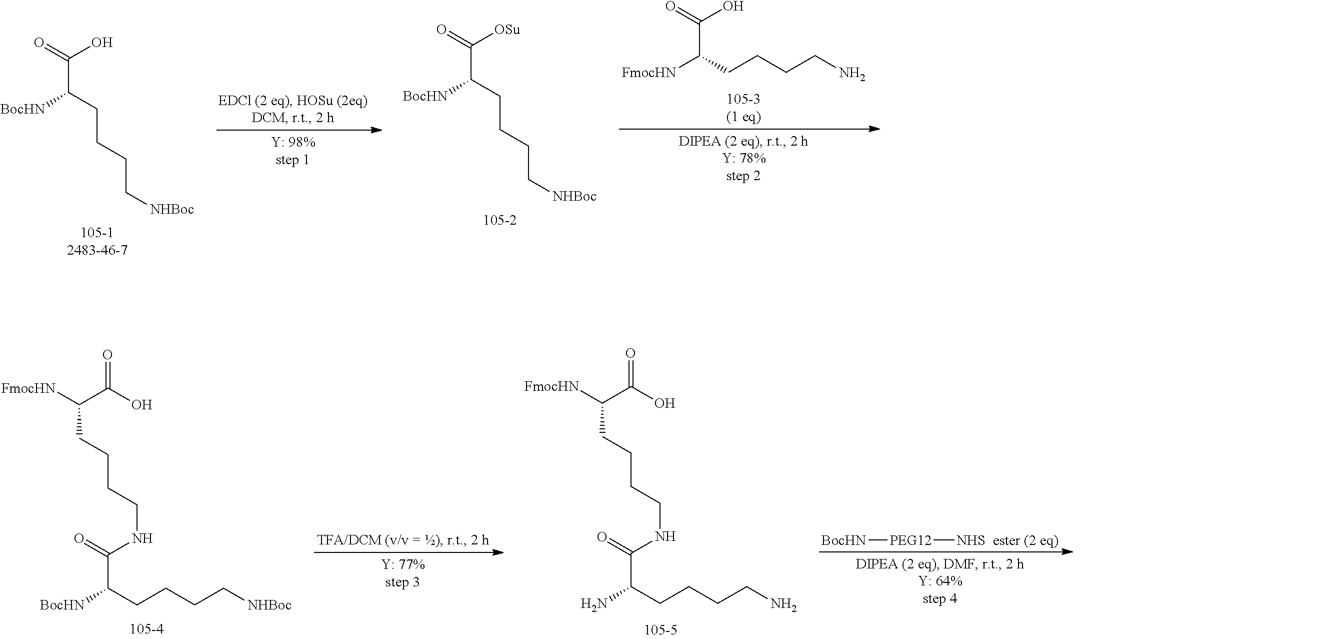

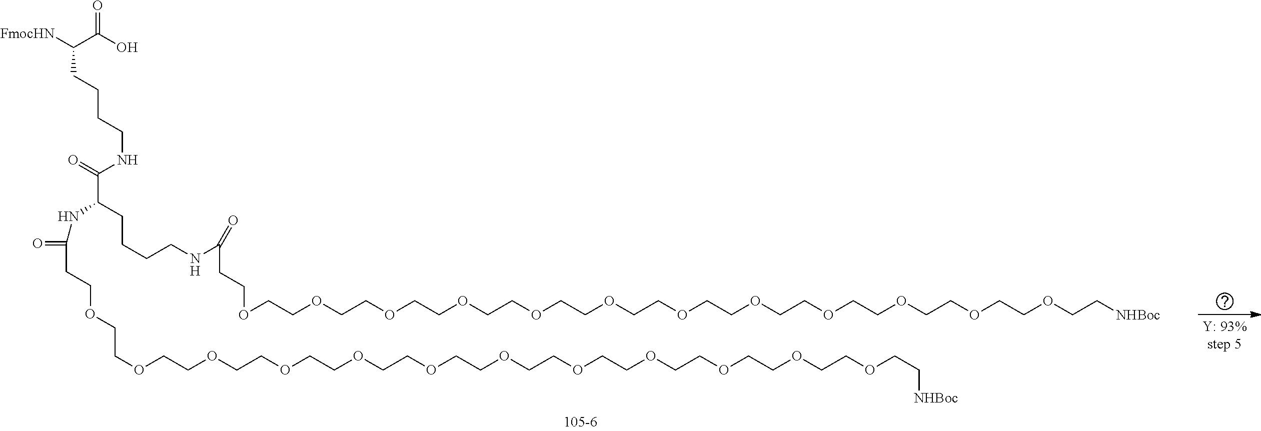

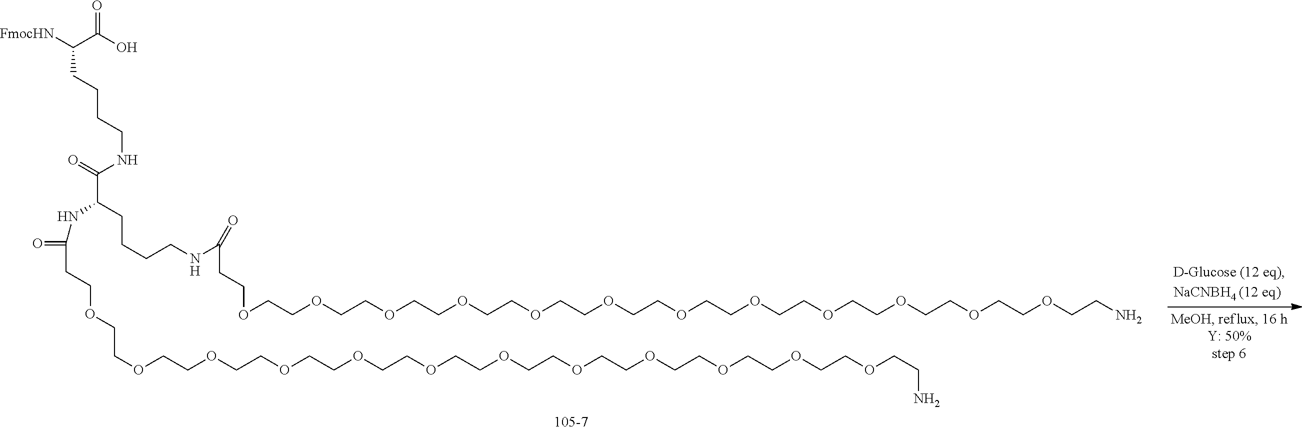

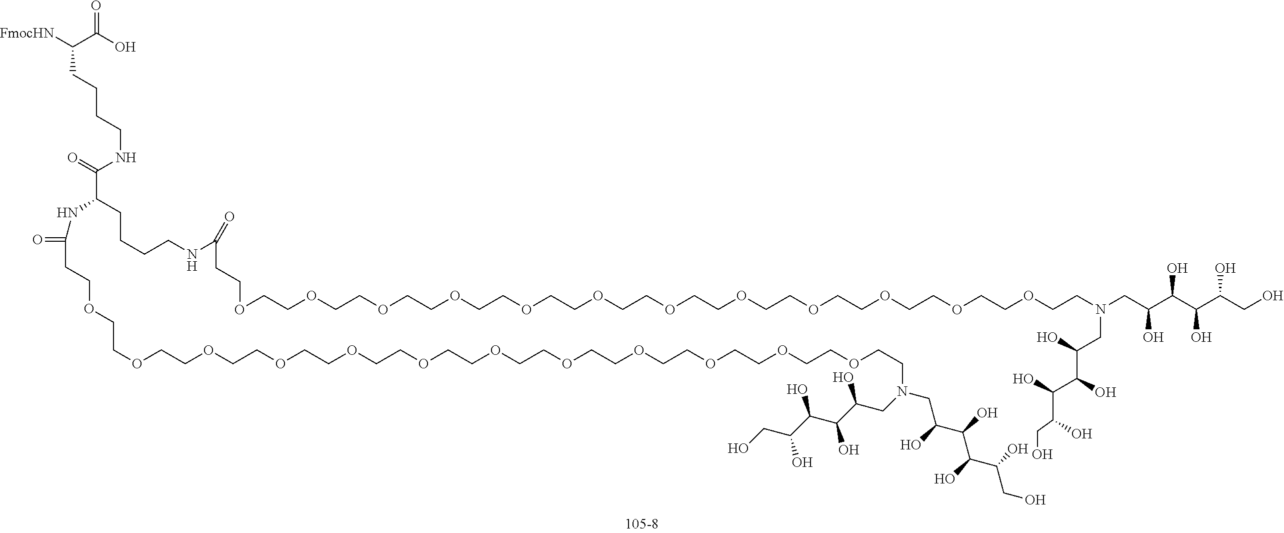

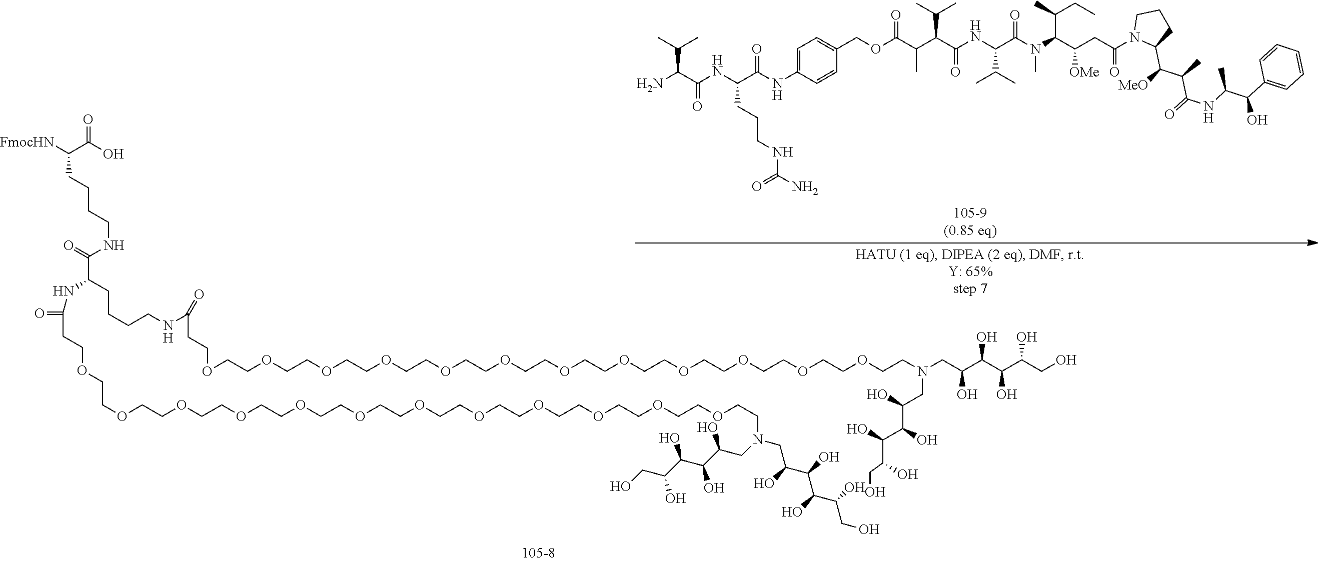

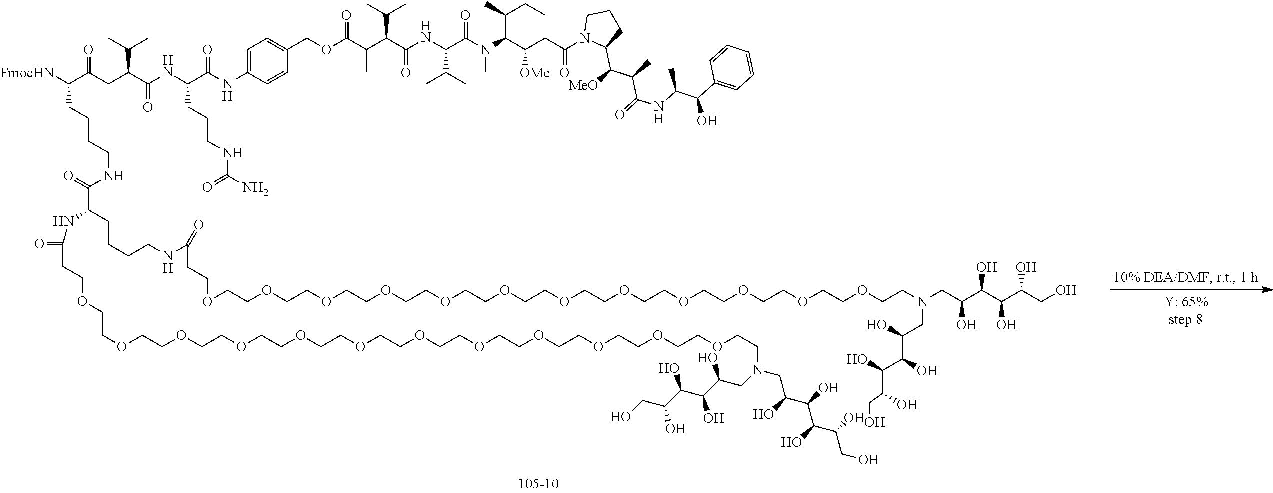

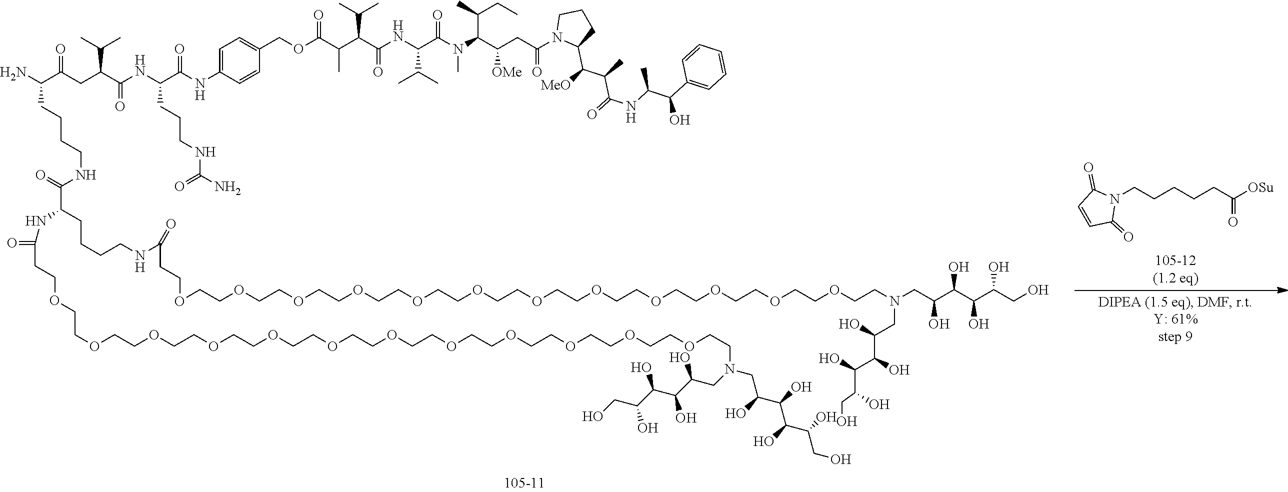

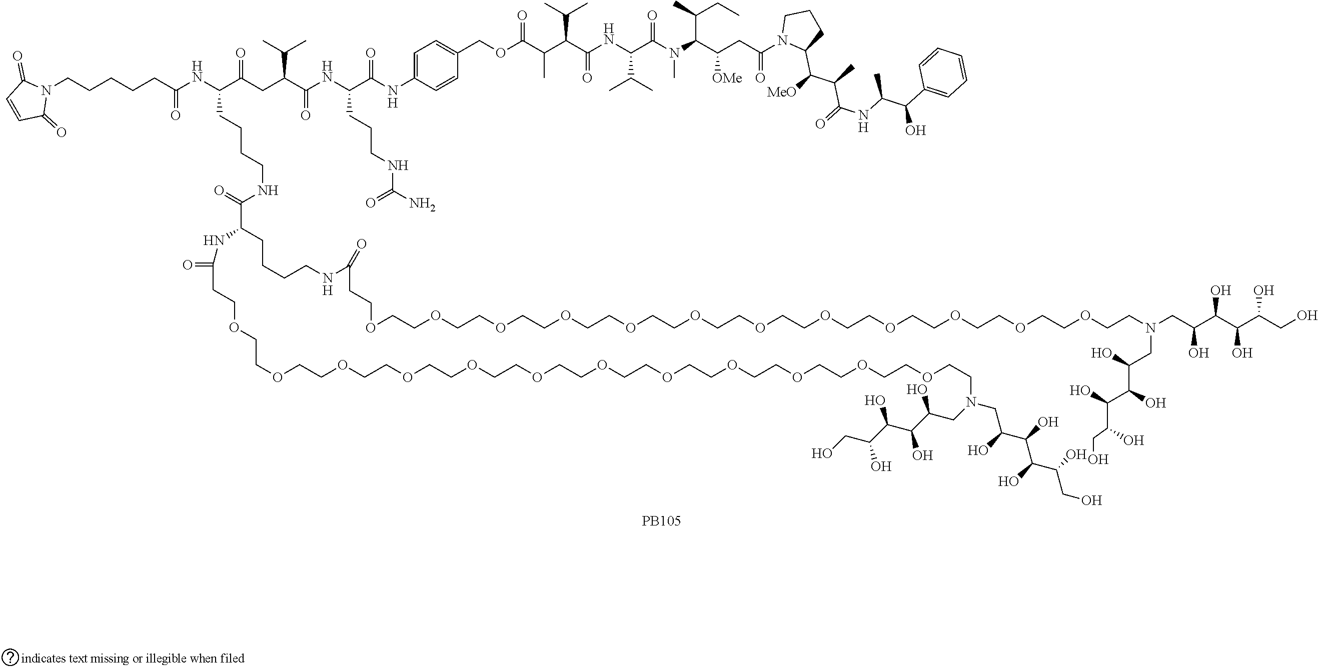

A Drug-Linker containing a PEG unit and a cleavable linker attached to MMAE (PB105) was prepared as follows:

### Step 1

A solution of compound 105-1 ((2S)-2,6-bis({[(tert-butoxy)carbonyl]amino})hexanoic acid (105-1, 5.0 g, 14.433 mmol)), 1-hydroxypyrrolidine-2,5-dione (2.249 mL, 28.867 mmol) and EDCl (5.53 g, 28.867 mmol) in DCM (50 mL) was stirred at room temperature for 2 h. Then the reaction solution was diluted with DCM (50 mL) and washed with water (50 mL*2). The organic layer was collected and dried over sodium sulfate, filtered and the filtrate concentrated under vacuo to dryness, affording crude compound 105-2 ((S)-2,5-dioxopyrrolidin-1-yl 2,6-bis(tert-butoxycarbonylamino)hexanoate (105-2, 6.35 g, 14.318 mmol, 99.20%)), which was used for the next steps directly. ESI m/z: 466.3 (M+Na)⁺.

### Step 2

A solution of compound 105-2 ((S)-2,5-dioxopyrrolidin-1-yl 2,6-bis(tert-butoxycarbonylamino)hexanoate (105-2, 6.40 g, 14.44 mmol)), compound 105-3 ((2S)-6-amino-2-({[(9H-fluoren-9-yl)methoxy]carbonyl}amino)hexanoic acid (105-3, 5.32 g, 14.440 mmol)) and DIPEA (3.73 g, 28.880 mmol) in DMF (10 mL) was stirred at room temperature for 2 h. Then the reaction mixture was purified by reverse phase flash chromatography (0.01% TFA) to get the desired fractions, which were freeze-dried to yield compound 105-4 ((2S)-6-[(2S)-2,6-bis({[(tert-butoxy)carbonyl]amino})hexanamido]-2-({[(9H-fluoren-9-yl)methoxy]carbonyl}amino)hexanoic acid (105-4, 7.9 g, 11.337 mmol, 78.51%)) as a white solid. ESI m/z: 719.4(M+Na)⁺.

### Step 3

To a solution of compound 105-4 ((2S)-6-[(2S)-2,6-bis({[(tert-butoxy)carbonyl]amino})hexanamido]-2-({[(9H-fluoren-9-yl)methoxy]carbonyl}amino)hexanoic acid (105-4, 4.53 g, 6.501 mmol)) in DCM (20 mL) was added TFA (10 mL, 134.626 mmol) slowly. The mixture was stirred at room temperature for 2 hour. Then the reaction solution was concentrated and the crude mixture was purified by reverse phase flash chromatography (0.01% TFA) to yield compound 105-5 ((2S)-6-[(2S)-2,6-diaminohexanamido]-2-({[(9H-fluoren-9-yl)methoxy]carbonyl}amino)hexanoic acid (105-5, 2.5 g, 5.034 mmol, 77.44%)) as a colorless oil. ESI m/z: 497.3 (M+H)⁺.

### Step 4

A solution of crude compound 105-5 ((2S)-6-[(2S)-2,6-diaminohexanamido]-2-({[(9H-fluoren-9-yl)methoxy]carbonyl}amino)hexanoic acid (105-5, 1.94 g, 3.907 mmol)) in DMF (5 mL) was added dropwise to a solution of 2,5-dioxopyrrolidin-1-yl 1-{[(tert-butoxy)carbonyl]amino}-3,6,9,12,15,18,21,24,27,30,33,36-dodecaoxanonatriacontan-39-oate (5.7 g, 6.995 mmol) and DIPEA (1.01 g, 7.813 mmol) in DMF (10 mL). The resulting solution was stirred at room temperature for 2 hr tocompletion. The reaction mixture was purified by reverse phase flash chromatography (0.01% TFA) to get the desired fractions, which were freeze-dried to yield compound 105-6 ((2S)-6-[(2S)-2,6-bis(1-{[(tert-butoxy)carbonyl]amino}-3,6,9,12,15,18,21,24,27,30,33,36-dodecaoxanonatriacontan-39-amido)hexanamido]-2-({[(9H-fluoren-9-yl)methoxy]carbonyl}amino)hexanoic acid (105-6, 4.75 g, 2.505 mmol, 64.12%)) as a white solid. ESI m/z: 566.2 (M-200)/3+H)⁺, 970.6 (M/2+Na)⁺.

### Step 5

To a solution of compound 105-6 ((2S)-6-[(2S)-2,6-bis(1-{[(tert-butoxy)carbonyl]amino}-3,6,9,12,15,18,21,24,27,30,33,36-dodecaoxanonatriacontan-39-amido)hexanamido]-2-({[(9H-fluoren-9-yl)methoxy]carbonyl}amino)hexanoic acid (105-6, 4.75 g, 2.505 mmol)) in DCM (10 mL) was added TFA (5 mL, 67.313 mmol). Then the solution was stirred at room temperature for 2 hours, concentrated to remove organic solvent, and the crude residue was purified by reverse phase flash chromatography (0.01% TFA) to yield compound 105-7 ((2S)-6-[(2S)-2,6-bis(1-amino-3,6,9,12,15,18,21,24,27,30,33,36-dodecaoxanonatriacontan-39-amido)hexanamido]-2-({[(9H-fluoren-9-yl)methoxy]carbonyl}amino)hexanoic acid (105-7, 3.95 g, 2.329 mmol, 92.97%)) as a colorless oil. ESI m/z: 566.3 (M/3+H)⁺.

### Step 6

To a solution of compound 105-7 ((2S)-6-{[(2S)-2,6-bis(1-amino-3,6,9,12,15,18,21,24,27,30,33,36-dodecaoxanonatriacontan-39-amido)hexyl]amino}-2-({[(9H-fluoren-9-yl)methoxy]carbonyl}amino)hexanoic acid (105-7, 3.95 g, 2.329 mmol)) in MeOH (90 mL) was added D-glucose (5.04 g, 27.948 mmol) in portions, and the mixture was heated under reflux for 30 minutes under a N₂ atmosphere. Then a solution of NaCNBH₃ (1.76 g, 27.948 mmol) in MeOH (10 mL) was added dropwise. The reaction mixture was stirred at this temperature for 18h. Then the reaction solution was purified by reverse phase flash chromatography (0.01% TFA) to get the desired fractions, which were freeze-dried to yield compound 105-8 ((2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-({[(9H-fluoren-9-yl)methoxy]carbonyl}amino)hexanoic acid (105-8, 4.13 g, 1.755 mmol, 75.36%)) as a colorless oil. ESI m/z: 785.0(M/3+H)⁺.

### Step 7

A solution of compound 105-8 ((2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-({[(9H-fluoren-9-yl)methoxy]carbonyl}amino)hexanoic acid (105-8, 226.17 mg, 0.096 mmol)), compound 105-9 ({4-[(2S)-2-[(2S)-2-amino-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (105-9, 90 mg, 0.080 mmol)) and DIPEA (20.64 mg, 0.160 mmol) in anhydrous DMF (3 mL) was stirred at room temperature for 5 min to allow the starting acid to dissolve thoroughly in solvent, then a solution of HATU (36.55 mg, 0.096 mmol) in anhydrous DMF (1 mL) was added dropwise by syringe over 10 min. After addition, the resulting solution was stirred for another 2 h to achieve complete reaction. Then the reaction solution was purified directly by reverse phase flash chromatography (0.01% TFA) to give the desired fractions, which were lyophilized by LabConc to yield a TFA salt of compound 105-10 ((9H-fluoren-9-yl)methyl N-[(1S)-5-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-1-{[(1S)-1-{[(1S)-4-(carbamoylamino)-1-({4-[({[(1S)-1-{[(1 S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl](methyl)carbamoyl}oxy)methyl]phenyl}carbamoyl)butyl]carbamoyl}-2-methylpropyl]carbamoyl}pentyl]carbamate (105-10, 180 mg, 0.052 mmol, 65.06%)) as a white solid. ESI m/z: 865.5 (M/4+H)⁺. ¹H NMR (400 MHz, DMSO-d₆) δ 10.07 (s, 1H), 8.36-8.08 (m, 4H), 7.98-7.88 (m, 5H), 7.83-7.79 (m, 1H), 7.74-7.65 (m, 3H), 7.58-7.56 (m, 3H), 7.43-7.39 (m, 2H), 7.34-7.24 (m, 8H), 7.20-7.13 (m, 1H), 6.00 (t, J=5.2 Hz, 1H), 5.51-5.36 (m, 6H), 5.09-4.94 (m, 2H), 4.94-4.70 (m, 4H), 4.70-4.38 (m, 14H), 4.33-4.14 (m, 7H), 4.04-3.93 (m, 6H), 3.79-3.73 (m, 4H), 3.68-3.64 (m, 4H), 3.61-3.55 (m, 18H), 3.52-3.46 (m, 96H), 3.25-3.12 (m, 13H), 3.06-2.83 (m, 13H), 2.43-2.37 (m, 3H), 2.30-2.26 (m, 3H), 2.16-2.04 (m, 2H), 2.04-1.91 (m, 2H), 1.83-1.15 (m, 25H), 1.05-0.97 (m, 6H), 0.97-0.73 (m, 26H) ppm. One proton of carboxyl group in TFA was revealed.

### Step 8

A solution of compound 105-10 (4-((2S,5S,8S,15S,62S,63R,64R,65R)-8-((((9H-fluoren-9-yl)methoxy)carbonyl)amino)-62,63,64,65,66-pentahydroxy-5-isopropyl-4,7,14,21-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-60-((2S,3R,4R,5R)-2,3,4,5,6-pentahydroxyhexyl)-2-(3-ureidopropyl)-24,27,30,33,36,39,42,45,48,51,54,57-dodecaoxa-3,6,13,20,60-pentaazahexahexacontanamido)benzyl ((S)-1-(((S)-1-(((3S,4S,5S)-1-((S)-2-((1R,2R)-3-(((1R,2S)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (105-10, 180 mg, 0.052 mmol)) in DMF (1.8 mL) was stirred at room temperature and diethylamine (0.2 mL, 1.941 mmol) was added. The resulting solution was stirred for 1h until the reaction was completed. Most of diethyl amine was evaporated off under vacuo and the residue was purified by reverse phase flash chromatography (0.01% TFA) to give the expected fractions, which were lyophilized to yield a TFA salt of compound 105-11 (4-((2S,5S,8S,15S,62S,63R,64R,65R)-8-amino-62,63,64,65,66-pentahydroxy-5-isopropyl-4,7,14,21-tetraoxo-15-((42S,43R,44R,45R)-42,43,44,45,46-pentahydroxy-40-((2S,3R,4R,5R)-2,3,4,5,6-pentahydroxyhexyl)-4,7,10,13,16,19,22,25,28,31,34,37-dodecaoxa-40-azahexatetracontanamido)-60-((2S,3R,4R,5R)-2,3,4,5,6-pentahydroxyhexyl)-2-(3-ureidopropyl)-24,27,30,33,36,39,42,45,48,51,54,57-dodecaoxa-3,6,13,20,60-pentaazahexahexacontanamido)benzyl ((S)-1-(((S)-1-(((3S,4S,5S)-1-((S)-2-((1R,2R)-3-(((1R,2S)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (105-11, 110 mg, 0.034 mmol, 65.31%)) as a white solid. ESI m/z: 619.1(linker fragment, (M-717-26-18)/4+H))⁺, 809.9 (M/4+H)⁺. ¹H NMR (400 MHz, DMSO-d₆) δ 10.10 (s, 1H), 8.43 (d, J=8.4 Hz, 1H), 8.36-8.33 (m, 1H), 8.15-8.10 (m, 5H), 8.01 (d, J=7.6 Hz, 1H), 7.94-7.91 (m, 1.5H), 7.85-7.83 (m, 1H), 7.67(d, J=8.4 Hz, 0.5H), 7.59-7.57 (m, 2H), 7.35-7.24 (m, 6H), 7.20-7.13 (m, 1H), 6.07-6.05 (m, 1H), 5.53-5.38 (m, 6H), 5.09-4.94 (m, 2H), 4.94-4.42 (m, 18H), 4.30-4.13 (m, 4H), 4.04-3.93 (m, 6H), 3.85-3.81 (m, 1H), 3.81-3.73 (m, 5H), 3.73-3.64 (m, 5H), 3.61-3.55 (m, 18H), 3.55-3.48 (m, 88H), 3.39-3.29 (m, 10H), 3.24-3.12 (m, 12H), 3.06-2.83 (m, 11H), 2.44-2.38 (m, 3H), 2.38-2.22 (m, 4H), 2.15-2.05 (m, 2H), 2.05-1.94 (m, 2H), 1.85-1.64 (m, 6H), 1.64-1.41 (m, 6H), 1.41-1.16 (m, 12H), 1.05-0.97 (m, 6H), 0.97-0.75 (m, 26H) ppm. Three protons of carboxyl group in TFA were revealed.

### Step 9

A solution of compound 105-11 ({4-[(2S)-2-[(2S)-2-[(2S)-2-amino-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (105-11, 100 mg, 0.031 mmol)) and DIPEA (7.97 mg, 0.062 mmol) in anhydrous DMF (2 mL) was stirred at room temperature, then a solution of 2,5-dioxopyrrolidin-1-yl 6-(2,5-dioxo-2,5-dihydro-1H-pyrrol-1-yl)hexanoate (11.43 mg, 0.037 mmol) in anhydrous DMF (2 mL) was added dropwise by syringe over 5 min. After addition, the resulting solution was stirred for another 6h until the starting amine was substantially consumed. Then the resulting solution was purified directly by Prep-HPLC (0.01% TFA) to yield a TFA salt of PB105 ({4-[(2S)-2-[(2S)-2-[(2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-[6-(2,5-dioxo-2,5-dihydro-1H-pyrrol-1-yl)hexanamido]hexanamido]-3-methylbutanamido]-5-(carbamoylamino)pentanamido]phenyl}methyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (PB105, 65 mg, 0.019 mmol, 61.34%)) as a white solid. ESI m/z: 667.8 (linker fragment, (M-717-26-18)/4+H)⁺; 858.3 (M/4+H)⁺. Retention time 6.042 min (HPLC). ¹H NMR (400 MHz, DMSO-d6) δ 10.05 (s, 1H), 8.38-8.30 (m, 0.5H), 8.17-8.10 (m, 3.5H), 7.99-7.91 (m, 2.5H), 7.91-7.82 (m, 2H), 7.68-7.65 (m, 1.5H), 7.59-7.57 (m, 2H), 7.34-7.24 (m, 6H), 7.19-7.15 (m, 1H), 7.00 (s, 2H), 6.08-5.96 (m, 1H), 5.53-5.37 (m, 6H), 5.14-4.36 (m, 22H), 4.29-4.13 (m, 5H), 4.04-3.93 (m, 7H), 3.82-3.73 (m, 5H), 3.73-3.66 (m, 5H), 3.62-3.55 (m, 20H), 3.52-3.41 (m, 88H), 3.36-3.24 (m, 10 H), 3.24-3.12 (m, 12H), 3.12-2.83 (m, 11H), 2.43-2.35 (m, 3H), 2.29 (t, J=6.8 Hz, 2H), 2.14-1.92 (m, 6H), 1.83-1.46 (m, 16H), 1.46-1.16 (m, 12H), 1.05-0.97 (m, 6H), 0.89-0.75 (m, 26H) ppm. Two protons of carboxyl group in TFA were revealed.

### Example 37: Preparation of a Drug-Linker Containing a PEG Unit and a Cleavable Linker Attached to Exatecan (PB106)

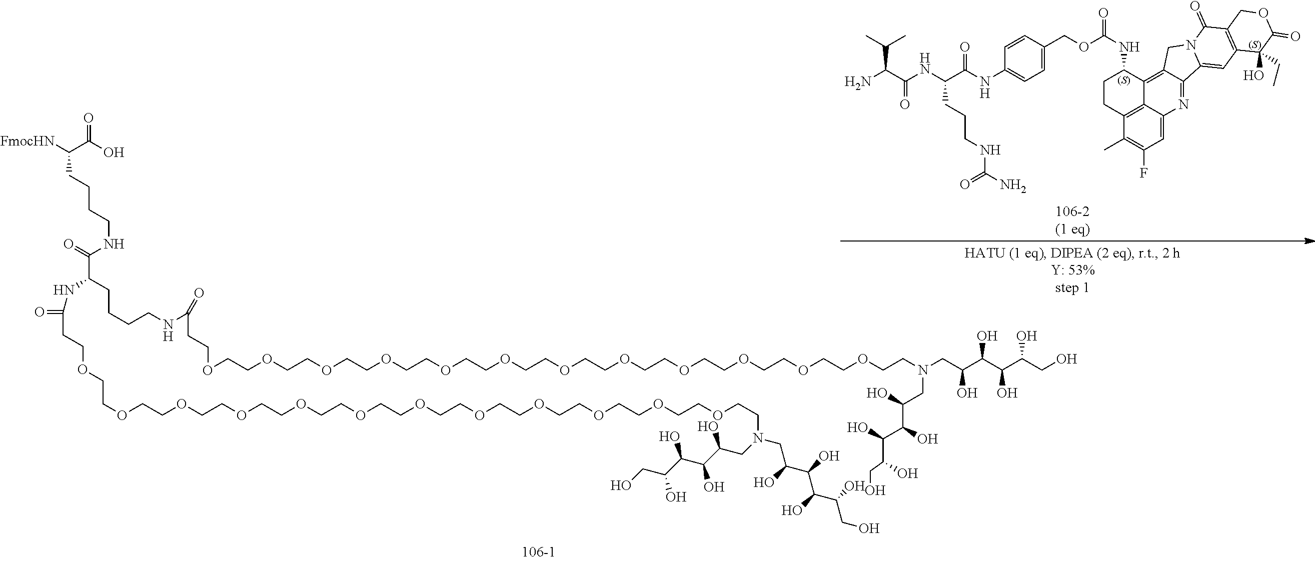

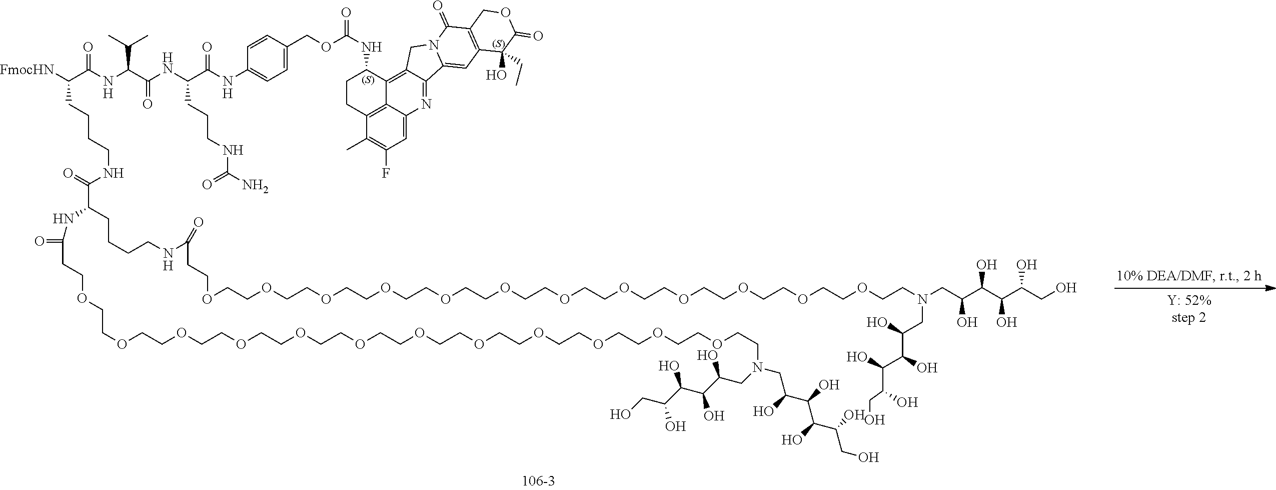

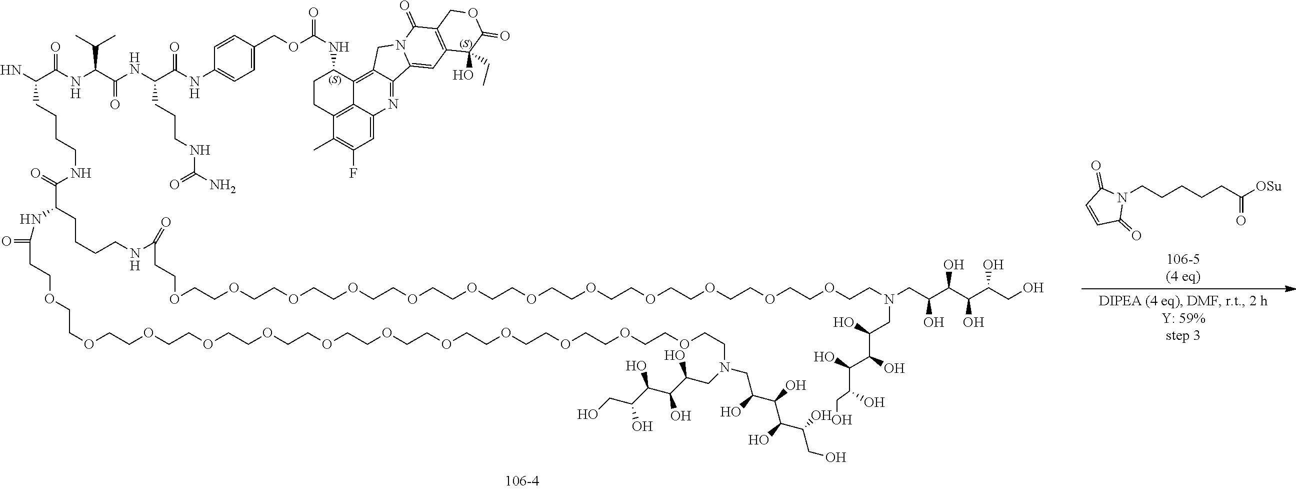

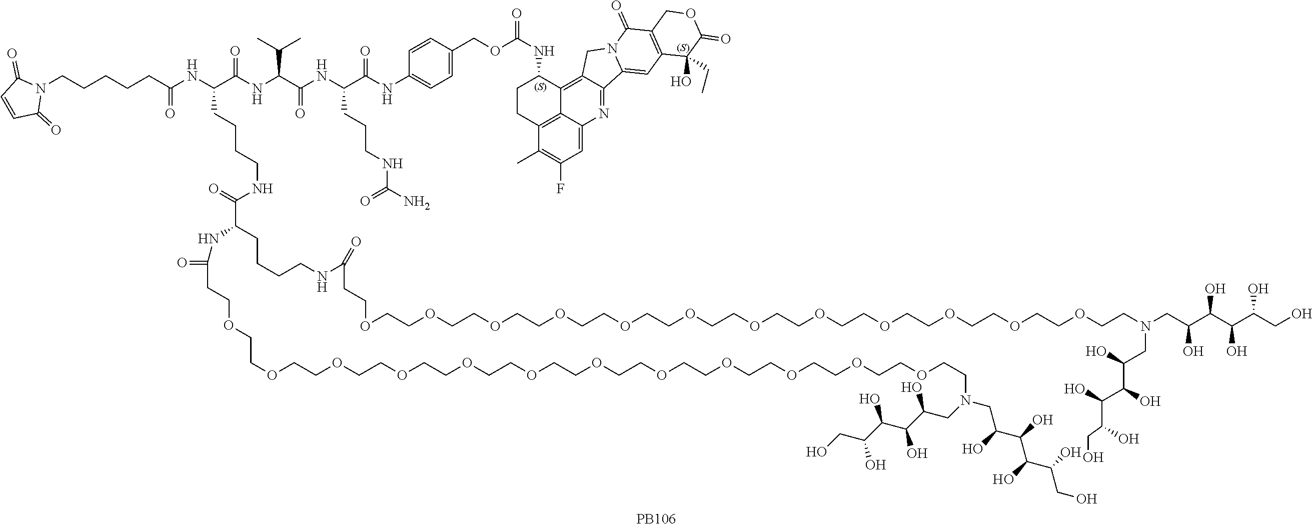

A Drug-Linker containing a PEG unit and a cleavable linker attached to exatecan (PB106) was prepared as follows:

### Step 1

A solution of compound 106-1 ((2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-({[(9H-fluoren-9-yl)methoxy]carbonyl}amino)hexanoic acid (106-1, 200 mg, 0.085 mmol)), compound 106-2 ({4-[(2S)-2-[(2S)-2-amino-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0\hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamate (106-2, 71.49 mg, 0.085 mmol)) and HATU (32.32 mg, 0.085 mmol), DIPEA (21.97 mg, 0.170 mmol) in DMF (2 mL) was stirred at room temperature for 2 h to completion. The reaction mixture was purified by reverse phase flash chromatography (0.01% TFA) to get the desired fractions, which were freeze-dried to yield compound 106-3 ((9H-fluoren-9-yl)methyl N-[(1S)-5-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-1-{[(1 S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0 \hat{ }{2,14}0.0\hat{ }{4,13}0.0\hat{ }{6,11}0.0\hat{ }{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (106-3, 144 mg, 0.045 mmol, 53.35%)) as a white solid. ESI m/z: 794.8 (M/4+H)⁺, 1059.3 (M/3+H)⁺.

### Step 2

A solution of compound 106-3 ((9H-fluoren-9-yl)methyl N-[(1S)-5-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-1-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0\hat{ }{2,14}0.0{4,13}0.0{6,11}0.0{20,24}]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}pentyl]carbamate (106-3, 144 mg, 0.045 mmol)) and diethylamine (0.2 mL, 0.180 mmol) in DMF (2 mL) was stirred at room temperature for 2 hour. Then the solution was purified by reverse phase flash chromatography (0.01% TFA) to yield compound 106-4 ({4-[(2S)-2-[(2S)-2-[(2S)-2-amino-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (106-4, 70 mg, 0.024 mmol, 52.29%)) as a white solid. ESI m/z: 985.3(M/3+H)⁺.

### Step 3

A solution of compound 106-4 ({4-[(2S)-2-[(2S)-2-[(2S)-2-amino-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (106-4, 40 mg, 0.014 mmol), 2,5-dioxopyrrolidin-1-yl 6-(2,5-dioxo-2,5-dihydro-1H-pyrrol-1-yl)hexanoate (106-5, 17.26 mg, 0.056 mmol)) and DIPEA (7.24 mg, 0.056 mmol) in DMF (2 mL) was stirred at room temperature for 2 h to completion. Then the reaction mixture was purified by Prep-HPLC (0.01% TFA) to get the desired fractions, which were freeze-dried by LabConc. to yield PB106-6 ({4-[(2S)-2-[(2S)-2-[(2S)-6-[(2S)-2,6-bis[(42S,43R,44R,45R)-42,43,44,45,46-pentahydroxy-40-[(2S,3R,4R,5R)-2,3,4,5,6-pentahydroxyhexyl]-4,7,10,13,16,19,22,25,28,31,34,37-dodecaoxa-40-azahexatetracontanamido]hexanamido]-2-[6-(2,5-dioxo-2,5-dihydro-1 H-pyrrol-1-yl)hexanamido]hexanamido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0 \hat{ }{2,14}0.0{4,13}0.0{6,11}0.0{20,24}]tetracosa-1,6(11),12,14,16,18,20(24)-heptaen-23-yl]carbamate (PB106, 26 mg, 0.008 mmol, 59.02%)) as a pale yellow solid. ESI m/z: 787.5 (M/4+H)⁺, 1049.9 (M/3+H)⁺. Retention time 5.626 min (HPLC). ¹H NMR (400 MHz, DMSO-d6) δ 10.05 (s, 1H), 8.22-8.05 (m, 4H), 8.01-7.93 (m, 2H), 7.89-7.77 (m, 3H), 7.66 (d, J=8.0 Hz, 1H), 7.60 (d, J=8.2 Hz, 2H), 7.36 (d, J=8.4 Hz, 2H), 7.32 (s, 1H), 7.00 (s, 2H), 6.56 (s, 1H), 6.01 (d, J=14.8 Hz, 1H), 5.50-5.39 (m, 8H), 5.33-5.24 (m, 3H), 5.08 (s, 2H), 4.88-4.67 (m, 4H), 4.67-4.38 (m, 12H), 4.38-4.13 (m, 4H), 4.00-3.94 (m, 4H), 3.83-3.77 (m, 4H), 3.72-3.63 (m, 4H), 3.63-3.56 (m, 18H), 3.55-3.46 (m, 88H), 3.38-3.14 (m, 9H), 3.14-2.91 (m, 10H), 2.38-2.30 (m, 5H), 2.28 (t, J=6.8 Hz, 2H), 2.23-2.08 (m, 5H), 2.02-1.83 (m, 4H), 1.72-1.53 (m, 5H), 1.46-1.28 (m, 13H), 1.28-1.12 (m, 7H), 0.90-0.80 (m, 9H) ppm. Two protons of carboxyl group from TFA were also revealed.

### Example 38: Preparation of a Drug-Linker Containing a PEG Unit and a Cleavable Linker Attached to Exatecan (PB107)

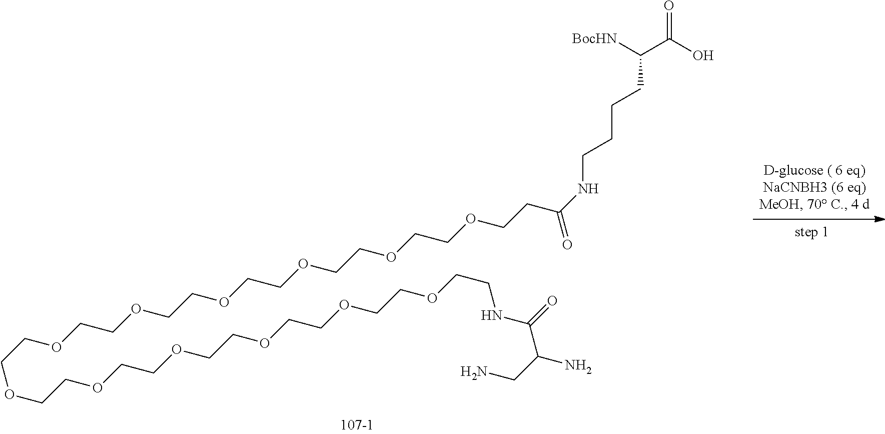 [SMILES: CC(C)(C)OC(=O)N[C@@H](CCCCNC(=O)CCOCCOCCOCCOCCOCCOCCOCCOCCOCCOCCOCCOCCNC(=O)C(N)CN)C(=O)O]

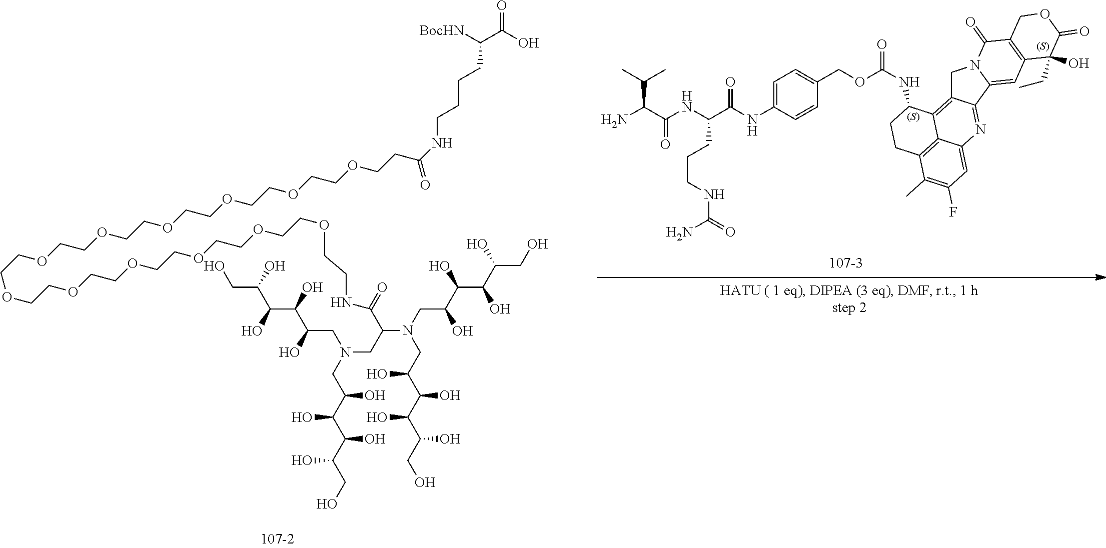

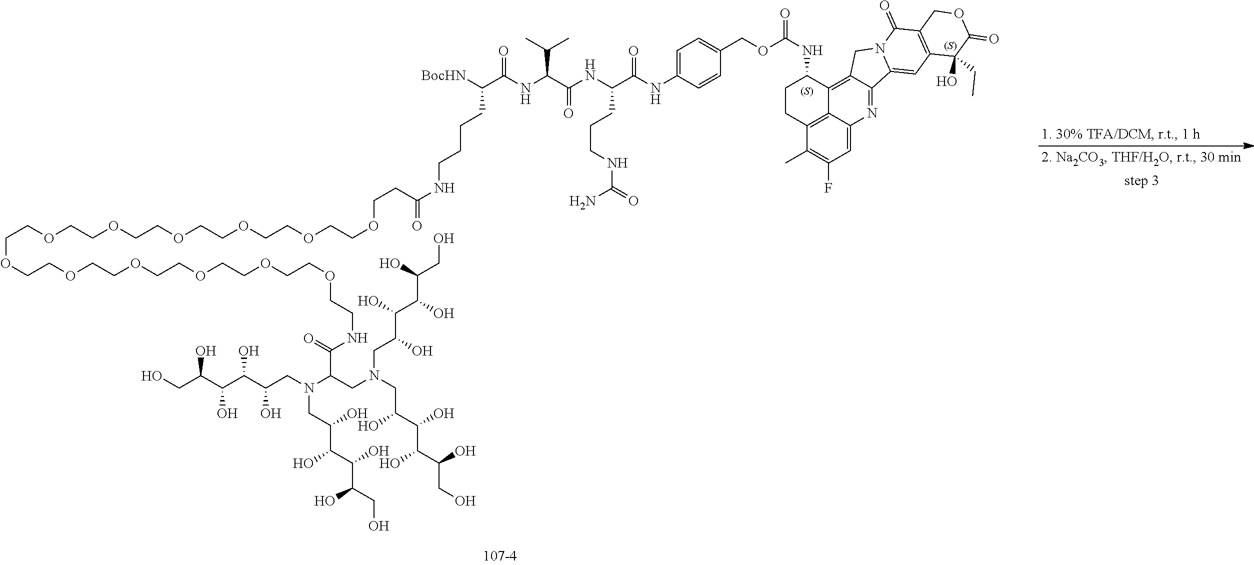

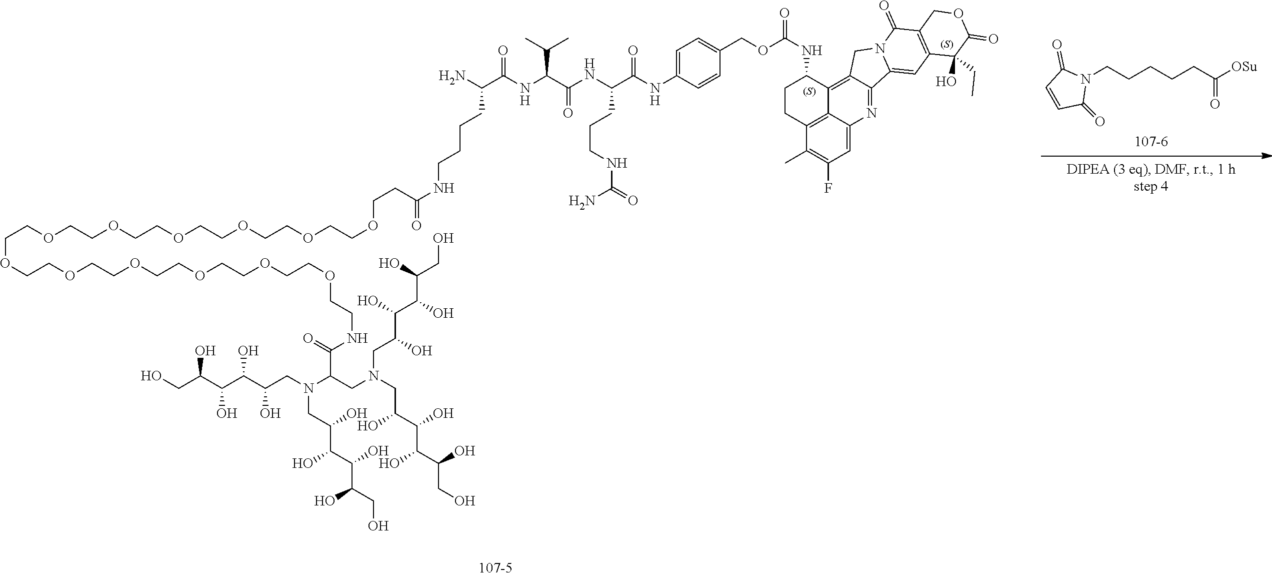

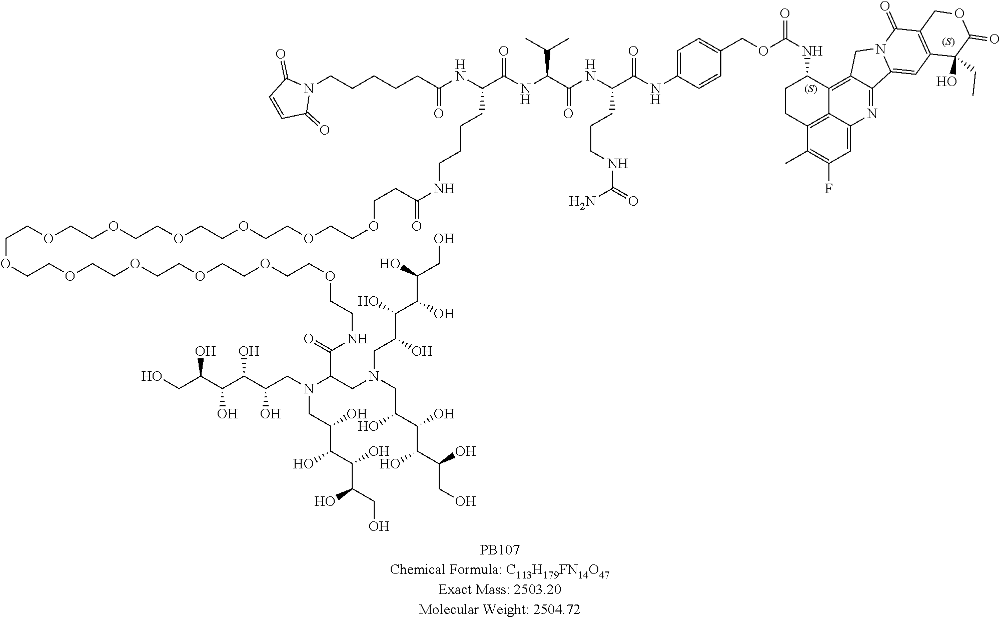

A Drug-Linker containing a PEG unit and a cleavable linker attached to exatecan (PB107) was prepared as follows:

### Step 1

A solution of compound 107-1 (0.5 g, 0.536 mmol) and D-glucose (0.77 g, 4.291 mmol) in anhydrous methanol (50 mL) was heated at 50° C. for 30 min, then NaCNBH₃ (0.27 g, 4.291 mmol) was added. The resulting solution was stirred for another 4 days at 70° C. until LCMS indicated all starting amine was consumed and the mass of desired product was detected. Then the reaction solution was concentrated and purified by reverse phase liquid chromatography to yield compound 107-2 (0.3 g, 0.189 mmol, 35.29%) as a white solid. purity=85%-90%.

### Step 2

A solution of compound 107-3 (0.16 g, 0.189 mmol), compound 107-2 (0.3 g, 0.189 mmol) and HATU (72 mg, 0.189 mmol) in anhydrous DMF (4 mL) was stirred at room temperature for 5 min, then DIPEA (74 mg, 0.567 mmol) was added. The resulting solution was stirred for another 1 hr at r.t. until LCMS indicated complete reaction. The reaction solution was purified directly by reverse phase liquid chromatography to yield compound 107-4 (140 mg, 0.058 mmol, 30.50%) as a white solid. purity=90%-95%.

### Step 3

A solution of compound 107-4 (140 mg, 0.058 mmol) and TFA (1 mL) in anhydrous DCM (4 mL) was stirred at room temperature for 1 hr until LCMS indicated all starting amine was consumed and the desired product (m/z=1156=2311/2+H) along with sugar-esterificated product (TFA was condensed with hydroxy group in sugar unit, mono-ester with m/z=(2311+96)/3+H=803) were formed. The completed reaction solution was condensed to dryness and then redissolved in THF (4 mL) and Water (2 mL), treated with saturated aqueous sodium carbonate solution to adjust pH to 8-9. The resulting solution was stirred at room temperature for 30 min to achieve complete hydrolysis. The solution was then treated with diluted HCl to adjust pH to 2-3 and condensed, the residue was purified by reverse phase liquid chromatography to yield compound 107-5 (113 mg, 0.049 mmol, 84.32%) as a white solid. purity=90%-95%.

### Step 4

A solution of compound 107-5 (113 mg, 0.049 mmol) and DIPEA (19 mg, 0.147 mmol) in anhydrous DMF (2 mL) was stirred at room temperature for 5 min, then a solution of compound 107-6 (23 mg, 0.074 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB107 (12 mg, 0.005 mmol, 9.84%) as a white solid. LCMS, m/z=835.93(M/3+H)⁺.

### Example 39: Preparation of a Drug-Linker Containing a PEG Unit and a Cleavable Linker Attached to Exatecan (PB108)

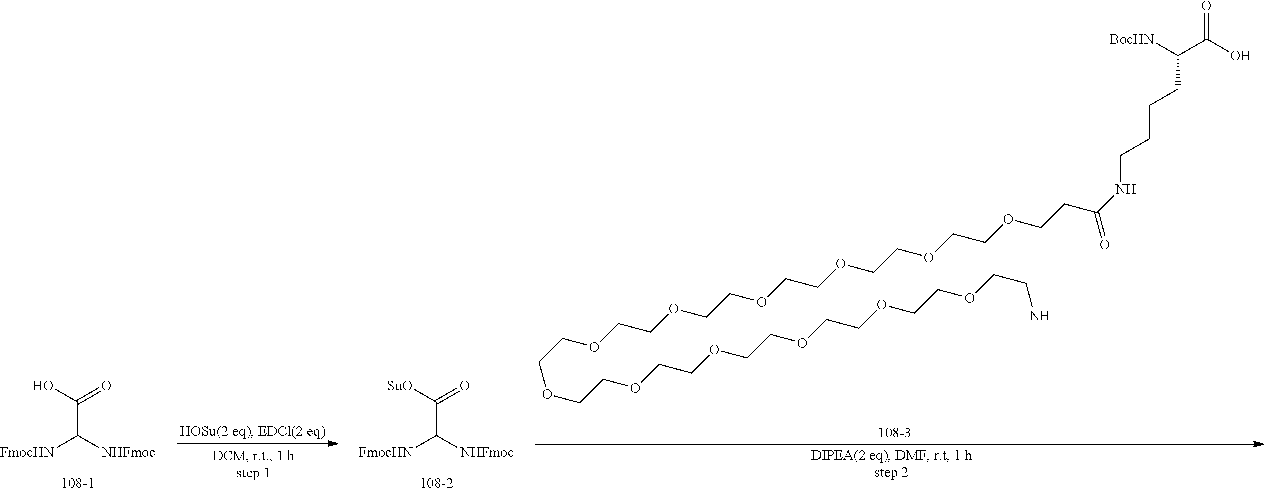

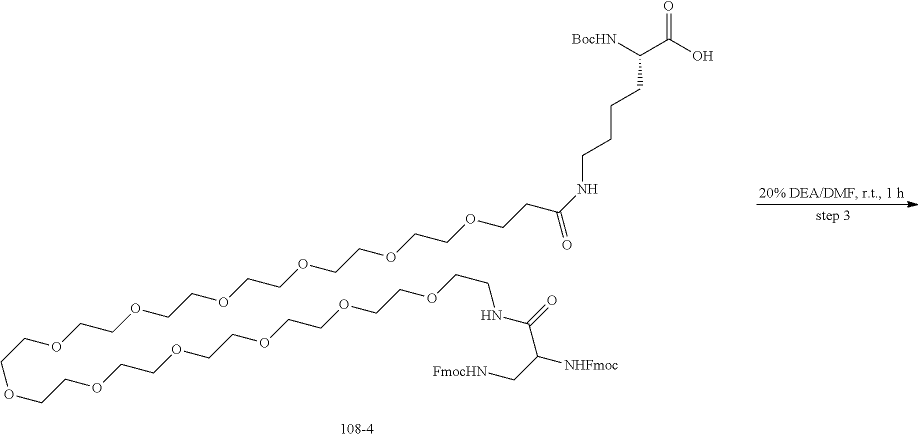

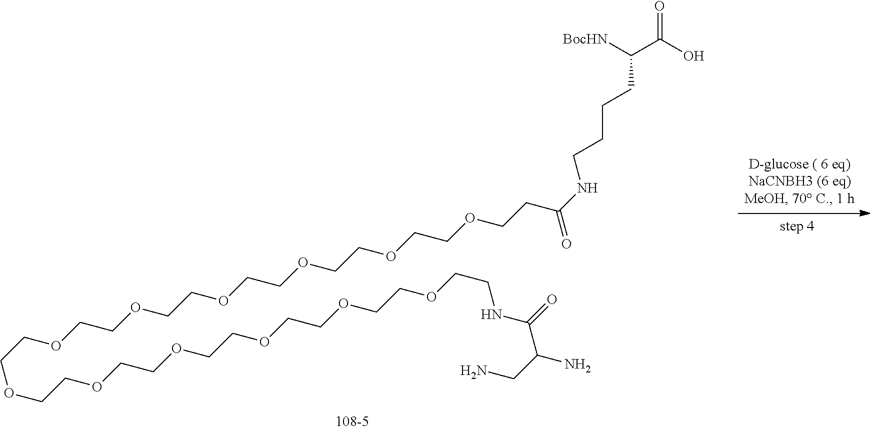 [SMILES: CC(C)(C)OC(=O)N[C@@H](CCCCNC(=O)CCOCCOCCOCCOCCOCCOCCOCCOCCOCCOCCOCCOCCNC(=O)C(N)CN)C(=O)O]

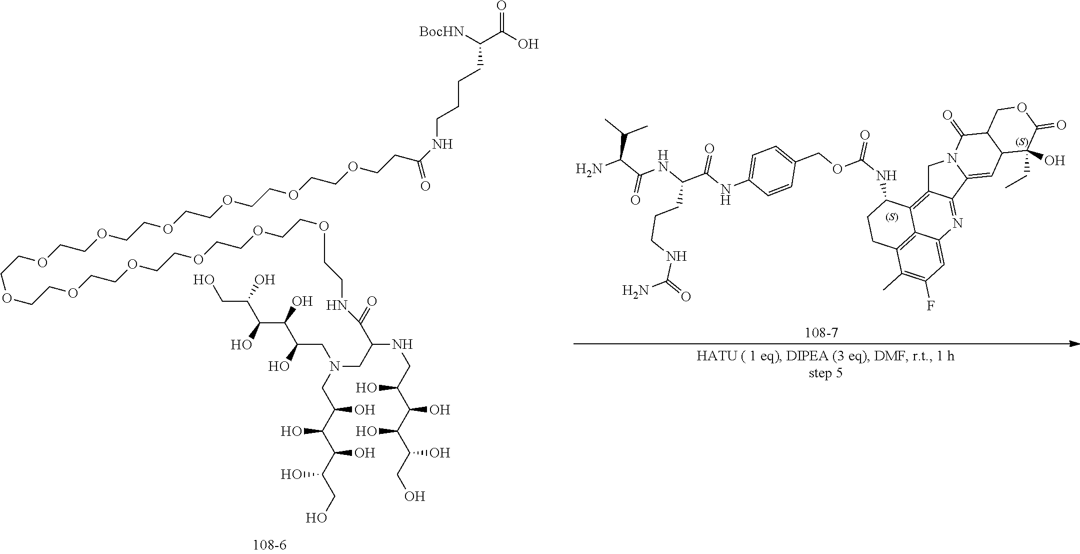

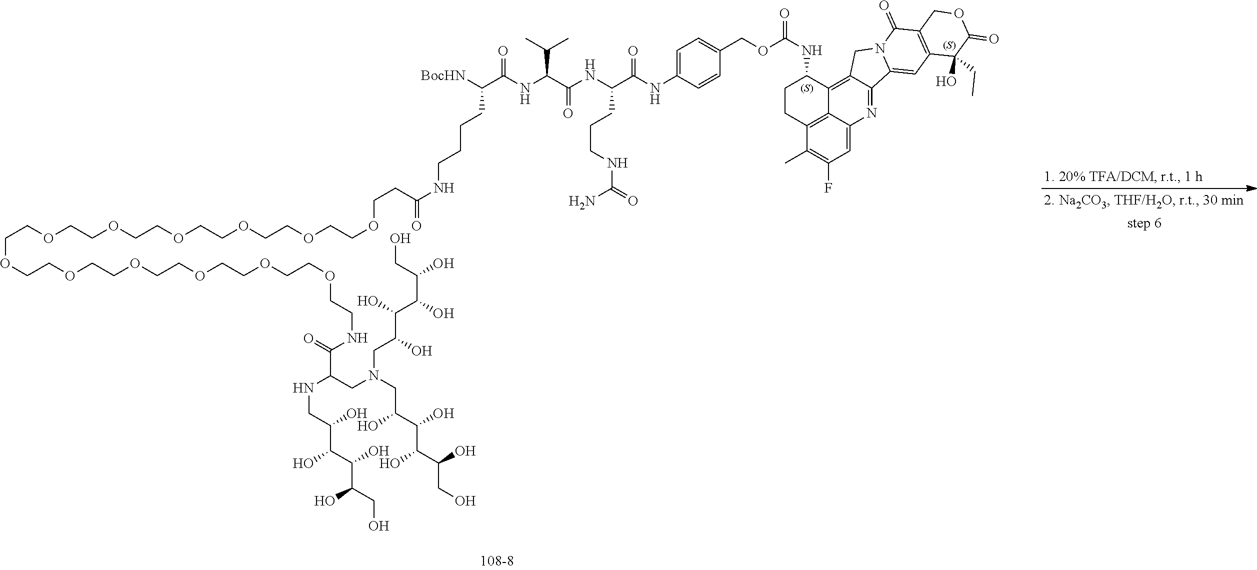

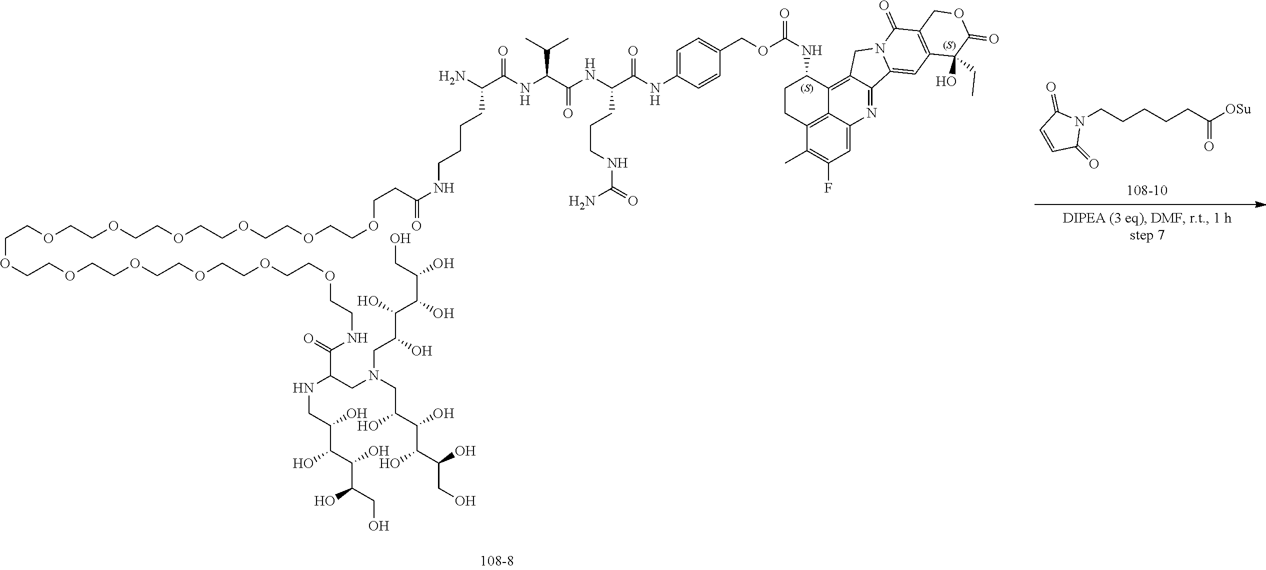

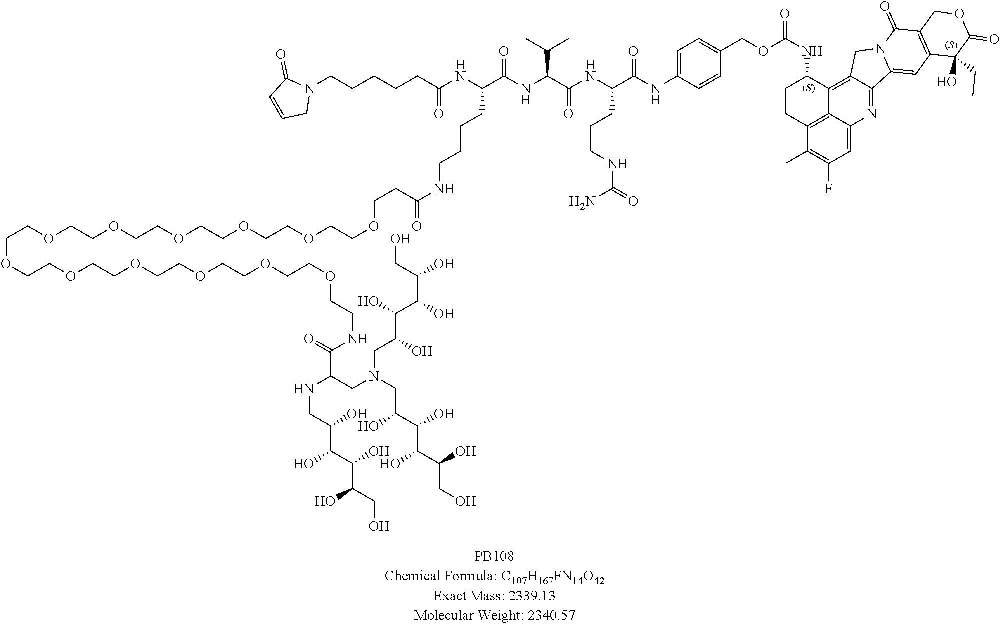

A Drug-Linker containing a PEG unit and a cleavable linker attached to exatecan (PB107) was prepared as follows:

### Step 1

A solution of compound 108-1 (2 g, 3.646 mmol) and HOSu (0.84 g, 7.291 mmol) in anhydrous DCM (30 mL) was stirred at room temperature, then EDCl (1.4 g, 7.291 mmol) was added. The resulting solution was stirred for another 1 hr at r.t. until LCMS indicated all starting amine was consumed and the desired product was detected. The resulting solution was washed with water, the organic layer was collected, and then water phase was extracted with DCM (40 mL*2). The combined organic layer was dried over sodium sulfate and filtered, concentrated to dryness to yield compound 108-2 (1.8 g, 2.788 mmol, 76.60%) as a white solid, which was used as such in the next step. purity=85%-90%.

### Step 2

A solution of compound 108-2 (0.76 g, 1.182 mmol) and compound 108-3 (1 g, 1.182 mmol) in anhydrous DMF (5 mL) was stirred at room temperature, then DIPEA (0.3 g, 2.364 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 108-4 (1.4 g, 1.017 mmol, 85.89%) as a white solid. purity=90%-95%.

### Step 3

A solution of compound 108-4 (1.4 g, 1.017 mmol) and DEA (5 mL) in anhydrous DMF (20 mL) was stirred at room temperature for 1 hr until LCMS indicated all starting amine was consumed and the desired product was detected. Then the solution was concentrated to dryness to yield compound 108-5 (0.9 g, 0.966 mmol, 94.74%) as yellow oil, which was used as such in the next step. purity=90%-95%.

### Step 4

A solution of compound 108-5 (0.9 g, 0.966 mmol) and D-Glucose (1.04 g, 5.793 mmol) in anhydrous methanol (50 mL) was heated at 50° C. for 30 min, then NaCNBH₃ (0.36 g, 5.793 mmol) was added. The resulting solution was stirred for another 1 hr at 70° C. until LCMS indicated all starting amine was consumed and the mass of desired product was detected. Then the reaction solution was concentrated and purified by reverse phase liquid chromatography to yield compound 108-6 (0.7 g, 0.492 mmol, 50.72%) as a white solid. purity=85%-90%.

### Step 5

A solution of compound 108-7 (0.21 g, 0.250 mmol), compound 108-6 (0.36 g, 0.250 mmol) and HATU (93 mg, 0.250 mmol) in anhydrous DMF (4 mL) was stirred at room temperature for 5 min, then DIPEA (95 mg, 0.750 mmol) was added. The resulting solution was stirred for another 1 hr at r.t. until LCMS indicated complete reaction. The reaction solution was purified directly by reverse phase liquid chromatography to yield compound 108-8 (0.21 g, 0.093 mmol, 37.50%) as a white solid. purity=90%-95%.

### Step 6

A solution of compound 108-8 (0.21 g, 0.093 mmol) and TFA (1 mL) in anhydrous DCM (4 mL) was stirred at room temperature for 1 hr until LCMS indicated all starting amine was consumed and the desired product (m/z=1074=2147/2+H) along with a sugar-esterificated product (TFA was condensed with hydroxy group in sugar unit, mono-ester with m/z=(2147+96)/2+H=1122) were formed. The completed reaction solution was condensed to dryness and then redissolved in THF (4 mL) and Water (2 mL), treated with saturated aqueous sodium carbonate solution to adjust the pH to 8-9. The resulting solution was stirred at room temperature for 30 min to achieve complete hydrolysis. The solution was then treated with diluted HCl to adjust the pH to 2-3 and condensed, the residue was purified by reverse phase liquid chromatography to yield compound 108-9 (160 mg, 0.075 mmol, 80.65%) as a white solid. purity=90%-95%.

### Step 7

A solution of compound 108-9 (160 mg, 0.075 mmol) and DIPEA (28.8 mg, 0.224 mmol) in anhydrous DMF (2 mL) was stirred at room temperature for 5 min, then a solution of compound 108-10 (34.4 mg, 0.113 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB108 (15 mg, 0.006 mmol, 8.62%) as a white solid. LCMS, m/z=1171.24(M/2+H)⁺.

### Example 40: Preparation of a Drug-Linker containing a PEG unit and a cleavable linker attached to MMAE (PB109)

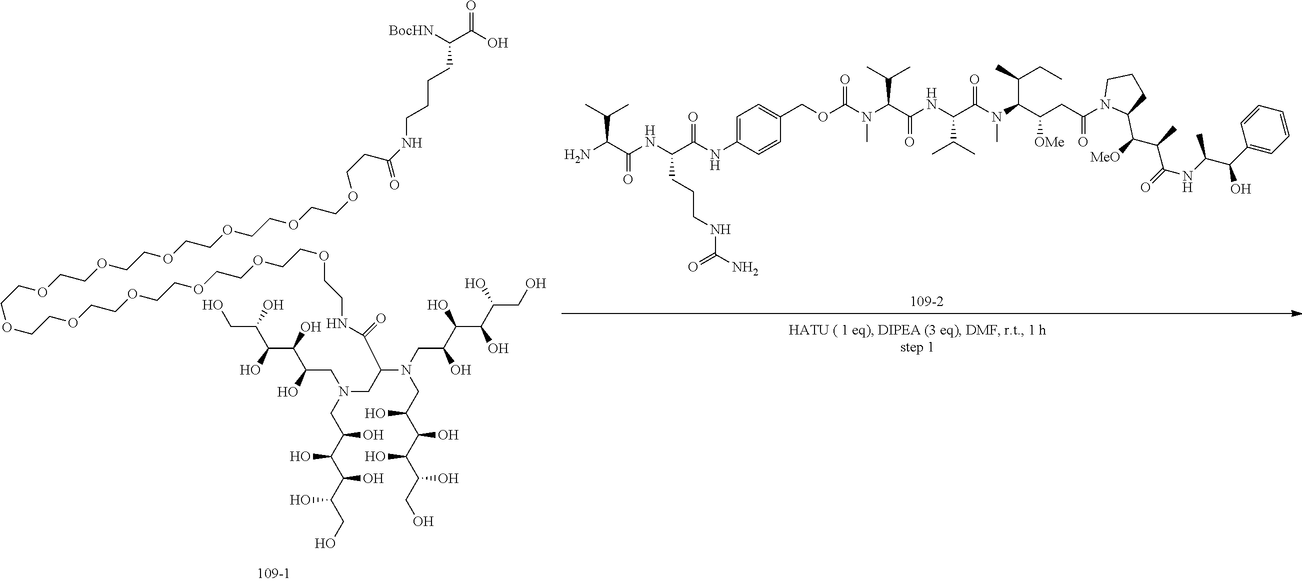 [SMILES: CC(C)(C)OC(=O)N[C@@H](CCCCNC(=O)CCOCCOCCOCCOCCOCCOCCOCCOCCOCCOCCOCCOCCNC(=O)C(CN(C[C@@H](O)[C@H](O)[C@@H](O)[C@@H](O)CO)C[C@@H](O)[C@H](O)[C@@H](O)[C@@H](O)CO)N(C[C@H](O)[C@@H](O)[C@H](O)[C@H](O)CO)C[C@H](O)[C@@H](O)[C@H](O)[C@H](O)CO)C(=O)O ; CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

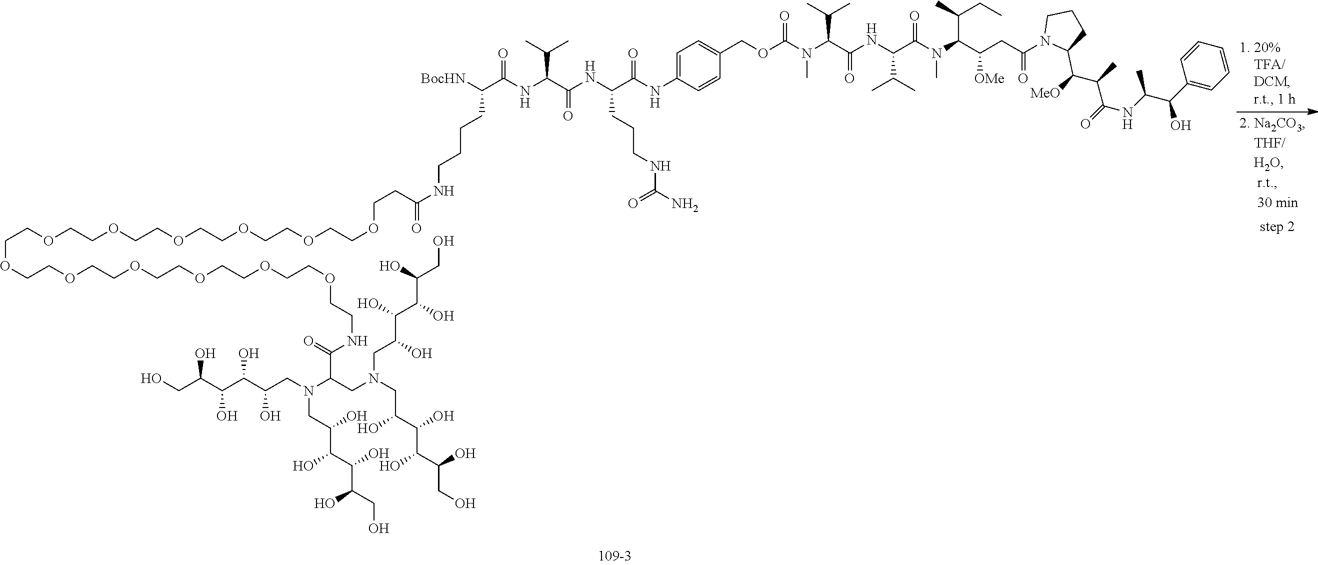 [SMILES: CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCNC(=O)C(CN(C[C@@H](O)[C@H](O)[C@@H](O)[C@@H](O)CO)C[C@@H](O)[C@H](O)[C@@H](O)[C@@H](O)CO)N(C[C@H](O)[C@@H](O)[C@H](O)[C@H](O)CO)C[C@H](O)[C@@H](O)[C@H](O)[C@H](O)CO)NC(=O)OC(C)(C)C)C(C)C)cc1)C(C)C]

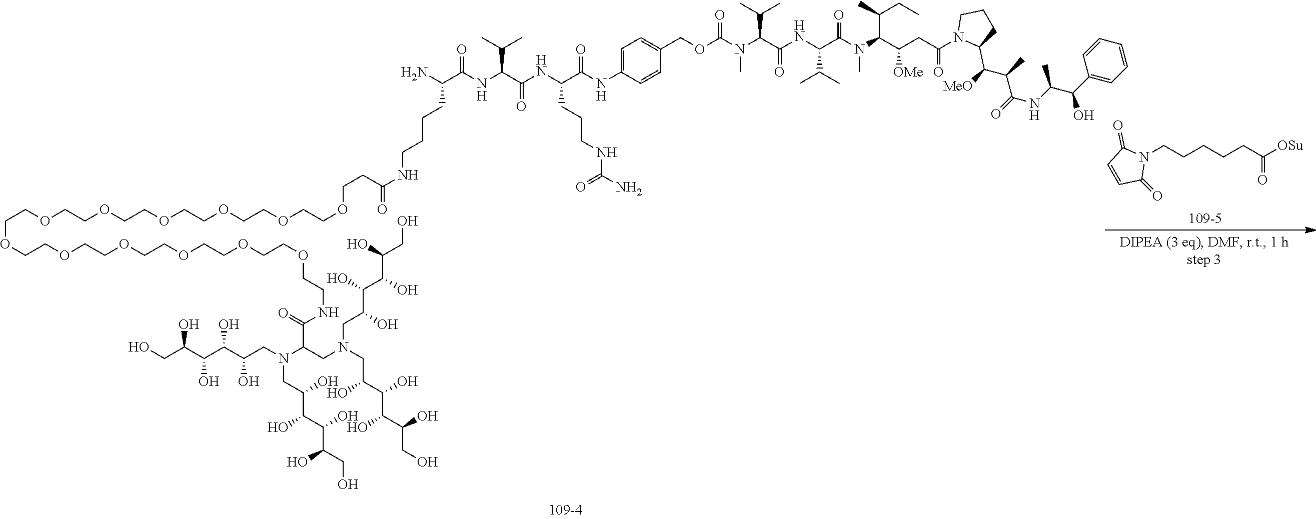

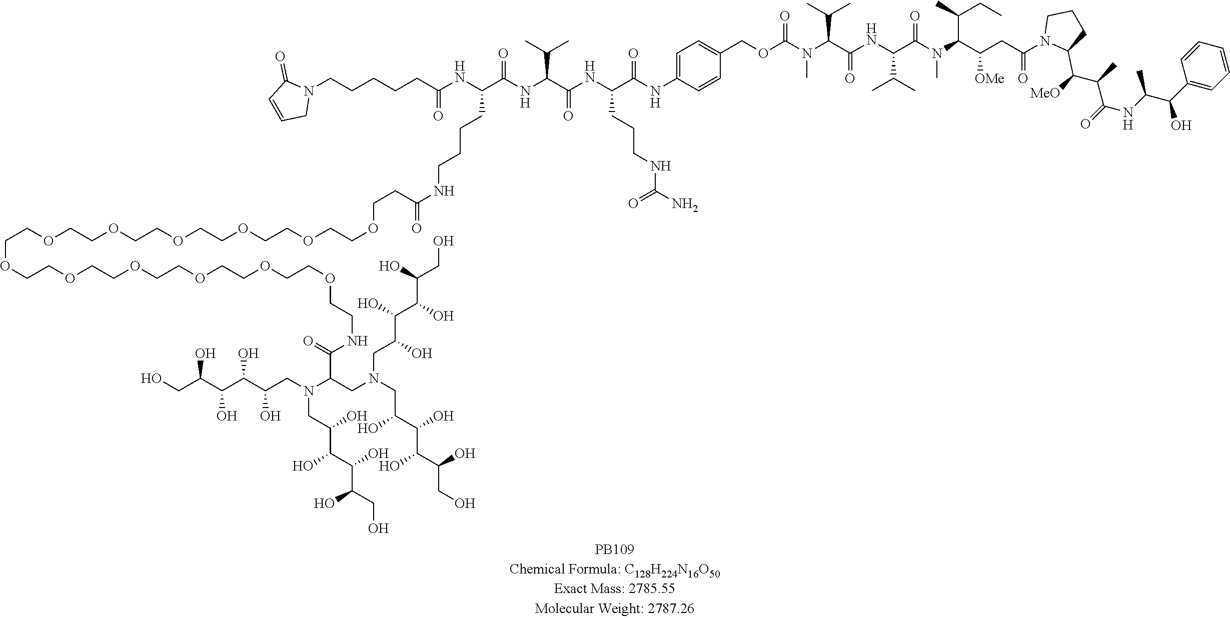 [SMILES: CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCNC(=O)C(CN(C[C@@H](O)[C@H](O)[C@@H](O)[C@@H](O)CO)C[C@@H](O)[C@H](O)[C@@H](O)[C@@H](O)CO)N(C[C@H](O)[C@@H](O)[C@H](O)[C@H](O)CO)C[C@H](O)[C@@H](O)[C@H](O)[C@H](O)CO)NC(=O)CCCCCN2CC=CC2=O)C(C)C)cc1)C(C)C]

A drug-Linker containing a PEG unit and a cleavable linker attached to MMAE (PB109) was prepared as follows:

### Step 1

A solution of compound 109-1 (170 mg, 0.107 mmol), compound 109-2 (120 mg, 0.107 mmol) and HATU (41 mg, 0.107 mmol) in anhydrous DMF (4 mL) was stirred at room temperature for 5 min, then DIPEA (41.4 mg, 0.321 mmol) was added. The resulting solution was stirred for another 1 hr at r.t. until LCMS indicated complete reaction. The reaction solution was purified directly by reverse phase liquid chromatography to yield compound 109-3 (103 mg, 0.038 mmol, 35.76%) as a white solid. purity=90%-95%.

### Step 2

A solution of compound 109-3 (103 mg, 0.038 mmol) and TFA (1 mL) in anhydrous DCM (4 mL) was stirred at room temperature for 1 hr until LCMS indicated all starting amine was consumed and the desired product (m/z=865=2594/3+H) along with sugar-esterificated product (TFA was condensed with hydroxy group in sugar unit, mono-ester with m/z=(2594+96)/3+H=897) were formed. The completed reaction solution was condensed to dryness and then redissolved in THF (4 mL) and water (2 mL), and treated with saturated aqueous sodium carbonate solution to adjust the pH to 8-9. The resulting solution was stirred at room temperature for 30 min to achieve complete hydrolysis. The solution was then treated with diluted HCl to adjust the pH to 6-7 and condensed; the residue was purified by reverse phase liquid chromatography to yield compound 109-4 (45 mg, 0.017 mmol, 45.45%) as a white solid. purity=90%-95%.

### Step 3

A solution of compound 109-4 (45 mg, 0.017 mmol) and DIPEA (6.7 mg, 0.052 mmol) in anhydrous DMF (2 mL) was stirred at room temperature for 5 min, then a solution of 109-5 (8.0 mg, 0.026 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB109 (6 mg, 0.002 mmol, 12.41%) as a white solid. LCMS, m/z=930.16(M/3+H)⁺.

### Example 41: Preparation of a Drug-Linker Containing a PEG Unit and a Cleavable Linker Attached to MMAE (PB110 or LD110)

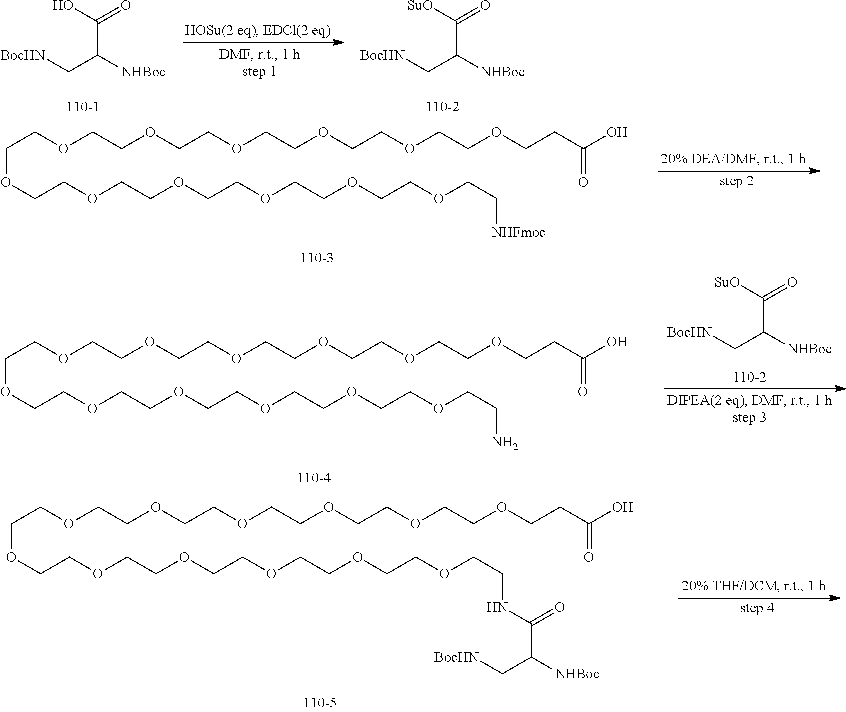

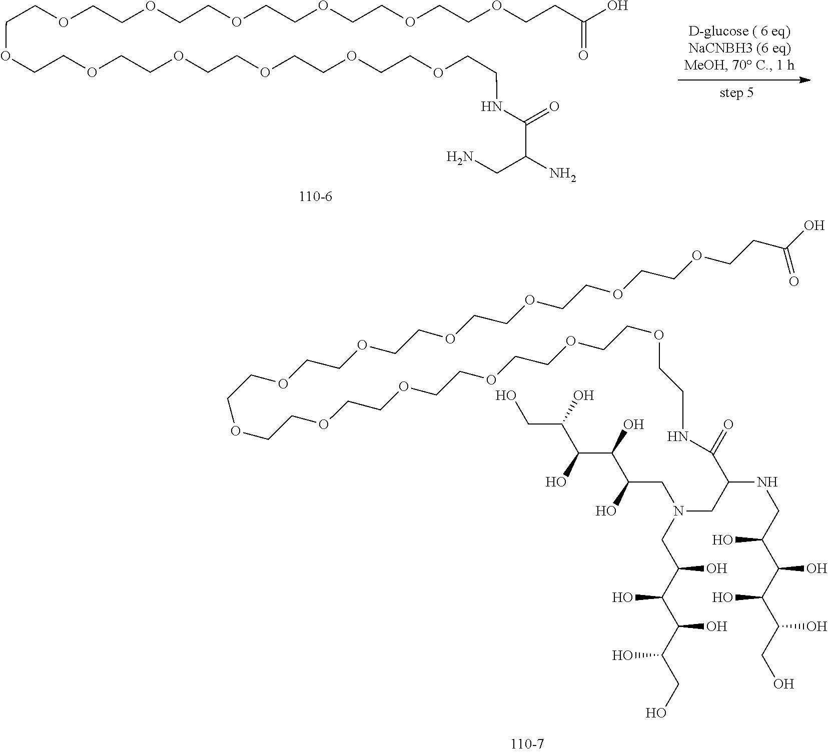 [SMILES: NCC(N)C(=O)NCCOCCOCCOCCOCCOCCOCCOCCOCCOCCOCCOCCOCCC(=O)O ; O=C(O)CCOCCOCCOCCOCCOCCOCCOCCOCCOCCOCCOCCOCCNC(=O)C(CN(C[C@@H](O)[C@H](O)[C@@H](O)[C@@H](O)CO)C[C@@H](O)[C@H](O)[C@@H](O)[C@@H](O)CO)NC[C@H](O)[C@@H](O)[C@H](O)[C@H](O)CO]

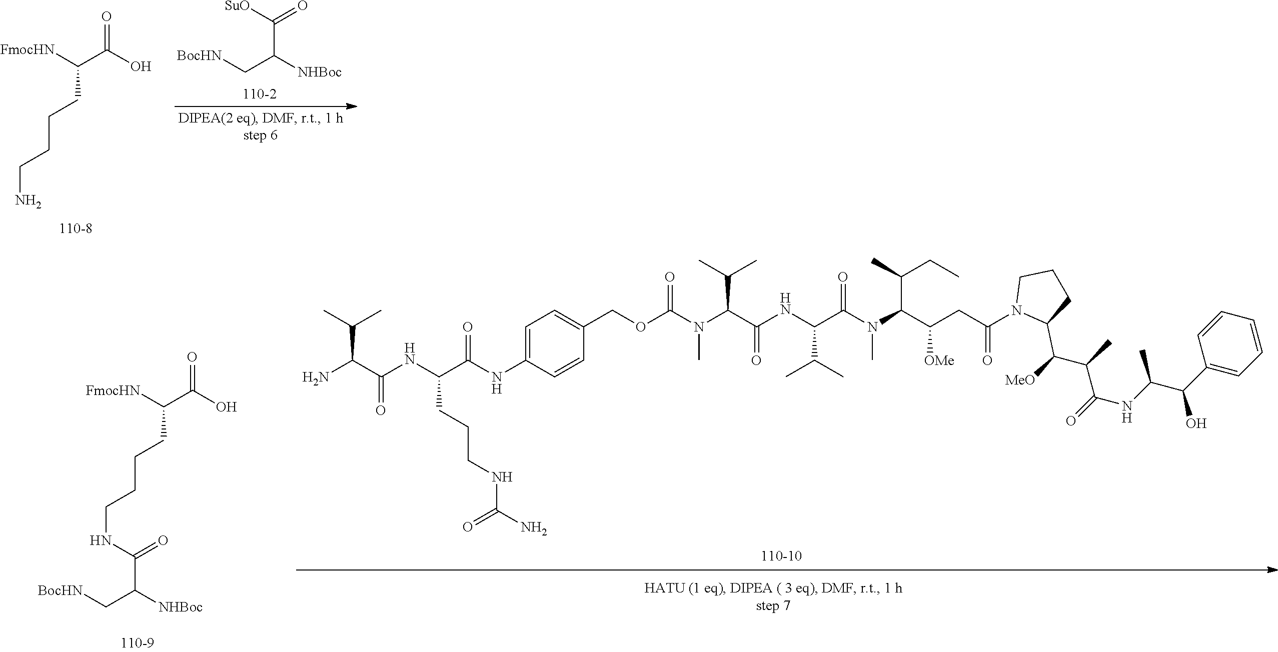

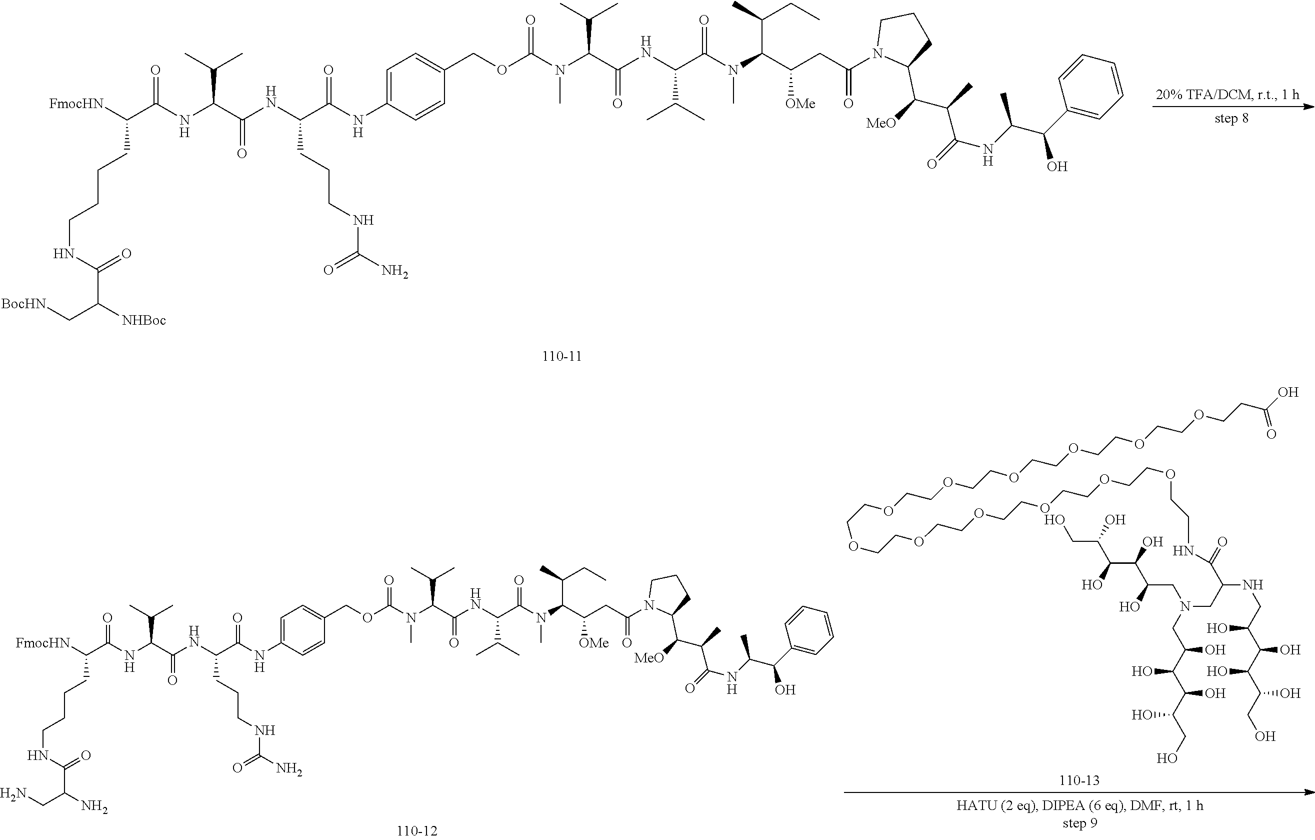

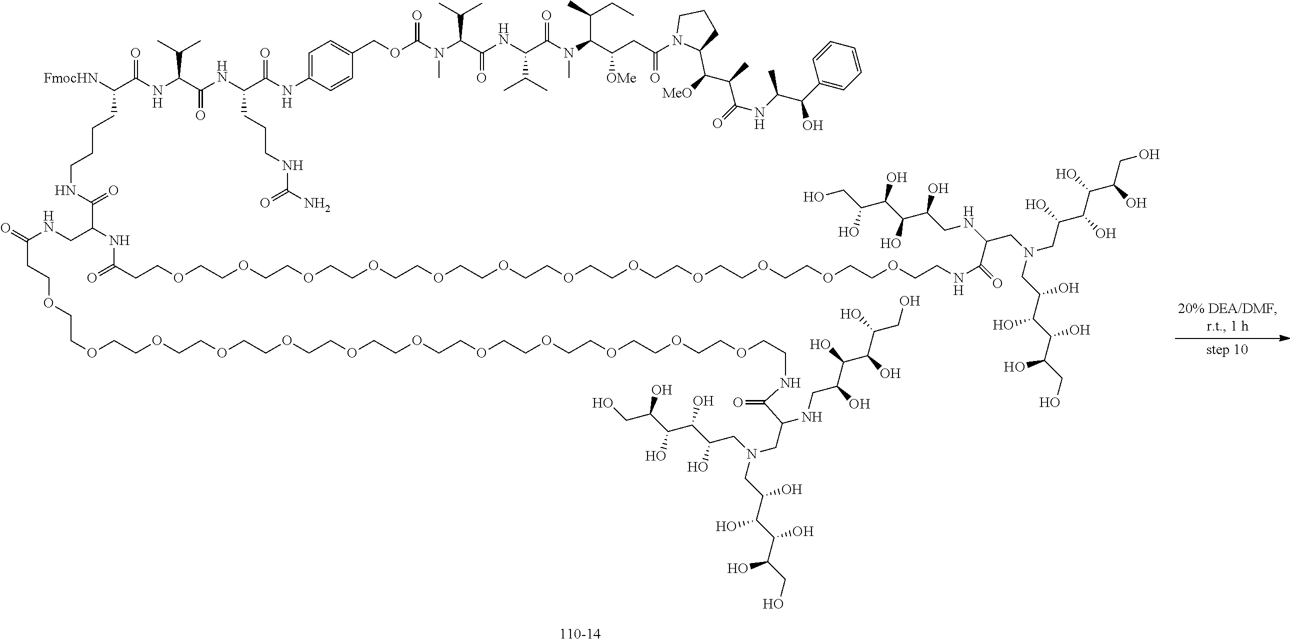

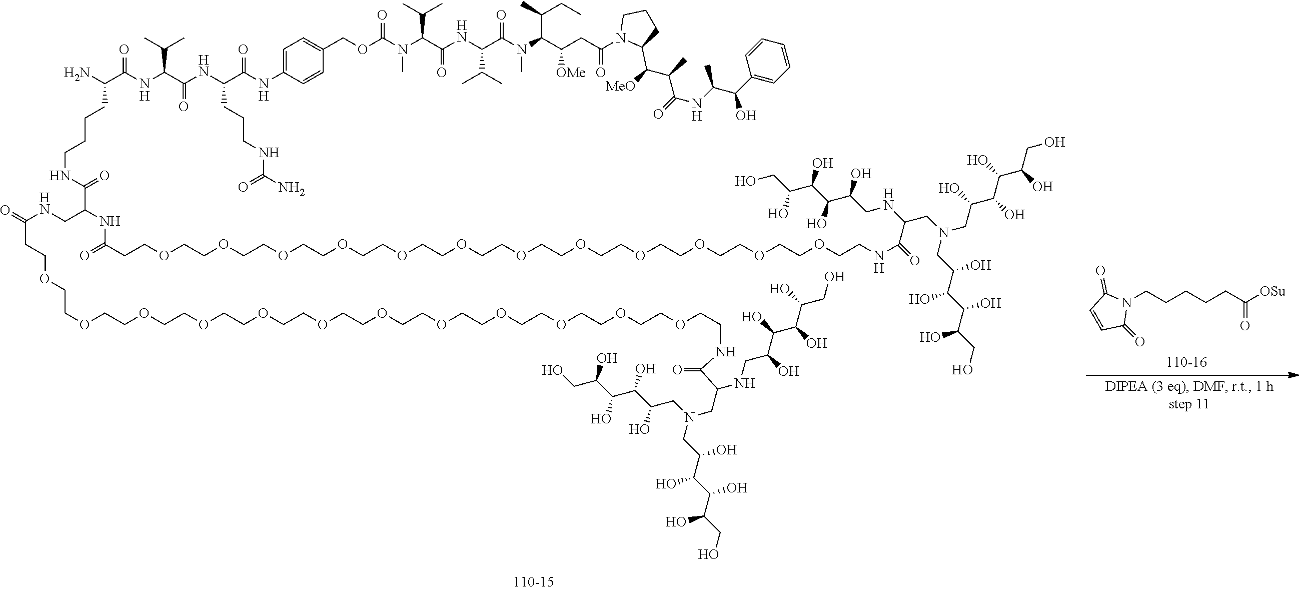

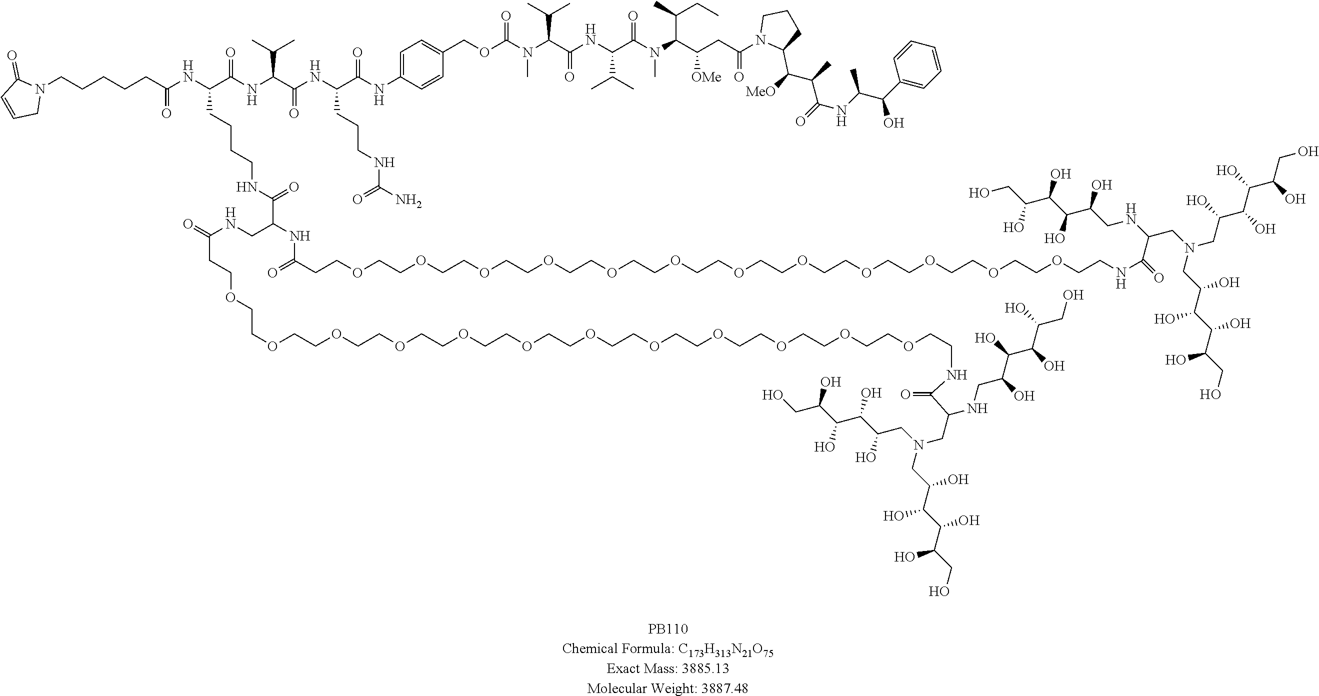

A Drug-Linker containing a PEG unit and a cleavable linker attached to MMAE (PB110 or LD110) was prepared as follows:

### Step 1

A solution of compound 110-1 (5 g, 8.671 mmol) and HOSu (2.0 g, 17.342 mmol) in anhydrous DCM (150 mL) was stirred at room temperature, then EDCl (3.3 g, 17.342 mmol) was added. The resulting solution was stirred for another 1 hr at r.t. until LCMS indicated all starting amine was consumed and the desired product was detected. The resulting solution was washed with water, the organic layer was collected, and then water phase was extracted with DCM (100 mL*2). The combined organic layer was dried over sodium sulfate and filtered, concentrated to dryness to yield compound 110-2 (4.7 g, 6.976 mmol, 80.48%) as a white solid, which was used as such in the next step. purity=85%-90%.

### Step 2

A solution of compound 110-3 (5 g, 5.953 mmol) and DEA (5 mL) in anhydrous DMF (20 mL) was stirred at room temperature for 1 hr until LCMS indicated all starting amine was consumed and the desired product was detected. Then the solution was concentrated to dryness to yield compound 110-4 (3.4 g, 5.504 mmol, 92.39%) as colorless oil, which was used as such in the next step. purity=90%-95%.

### Step 3

A solution of compound 110-2 (1 g, 2.491 mmol) and compound 110-4 (1.5 g, 2.491 mmol) in anhydrous DMF (10 mL) was stirred at room temperature, then DIPEA (0.64 g, 4.982 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 110-5 (1.3 g, 1.438 mmol, 57.78%) as a colorless oil. purity=90%-95%.

### Step 4

A solution of compound 110-5 (1.3 g, 1.438 mmol) and TFA (4 mL) in anhydrous DCM (16 mL) was stirred at room temperature for 1 hr until LCMS indicated all starting amine was consumed and the desired product was detected. Then the solution was concentrated to dryness to yield compound 110-6 (910 mg, 1.293 mmol, 89.92%) as yellow oil, which was used as such in the next step. purity=85%-90%.

### Step 5

A solution of compound 110-6 (910 mg, 1.293 mmol) and D-glucose (1.4 g, 7.771 mmol) in anhydrous methanol (40 mL) was heated at 50° C. for 30 min, then NaCNBH₃ (488 mg, 7.766 mmol) was added. The resulting solution was stirred for another 1 hr at 70° C. until LCMS indicated all starting aminewas consumed and the mass of desired product was detected. Then the reaction solution was concentrated and purified by reverse phase liquid chromatography to yield compound 110-7 (320 mg, 0.268 mmol, 20.70%) as a white solid. purity=85%-90%.

### Step 6

A solution of compound 110-2 (1 g, 2.491 mmol) and compound 110-8 (0.92 g, 2.491 mmol) in anhydrous DMF (10 mL) was stirred at room temperature, then DIPEA (0.64 g, 4.982 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 110-9 (1.4 g, 2.138 mmol, 85.89%) as a white solid. purity=90%-95%.

### Step 7

A solution of compound 110-9 (0.58 g, 0.886 mmol), compound 110-10 (1 g, 0.890 mmol) and HATU (0.34 g, 0.894 mmol) in anhydrous DMF (10 mL) was stirred at room temperature for 5 min, then DIPEA (0.35 g, 2.708 mmol) was added. The resulting solution was stirred for another 1 hr at r.t. until LCMS indicated complete reaction. The reaction solution was purified directly by reverse phase liquid chromatography to yield compound 110-11 (240 mg, 0.136 mmol, 15.29%) as a white solid. purity=90%-95%.

### Step 8

A solution of compound 110-11 (240 mg, 0.136 mmol) and TFA (1 mL) in anhydrous DCM (4 mL) was stirred at room temperature for 1 hr until LCMS indicated all starting amine was consumed and the desired product was detected. Then the solution was concentrated to dryness to yield compound 110-12 (195 mg, 0.125 mmol, 91.68%) as a yellow oil, which was used as such in the next step. purity=85%-90%.

### Step 9

A solution of compound 110-12 (195 mg, 0.125 mmol), compound 110-13 (299 mg, 0.250 mmol) and HATU (95 mg, 0.250 mmol) in anhydrous DMF (5 mL) was stirred at room temperature for 5 min, then DIPEA (97 mg, 0.751 mmol) was added. The resulting solution was stirred for another 1 hr at r.t. until LCMS indicated complete reaction. The reaction solution was purified directly by reverse phase liquid chromatography to yield compound 110-14 (210 mg, 0.053 mmol, 42.89%) as a white solid. purity=90%-95%.

### Step 10

A solution of compound 110-14 (210 mg, 0.053 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 110-15 (175 mg, 0.047 mmol, 88.38%) as colorless oil, which was used as such in the next step. purity=90%-95%.

### Step 11

A solution of compound 110-15 (175 mg, 0.047 mmol) and DIPEA (12.2 mg, 0.094 mmol) in anhydrous DMF (2 mL) was stirred at room temperature for 5 min, then a solution of compound 110-16 (21.9 mg, 0.071 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB110 (73 mg, 0.019 mmol, 39.63%) as a white solid. LCMS, m/z=972.82(M/4+H)⁺.

### Example 42: Preparation of a Drug-Linker Containing a PEG Unit and a Cleavable Linker Attached to Exatecan (PB111 or LD111)

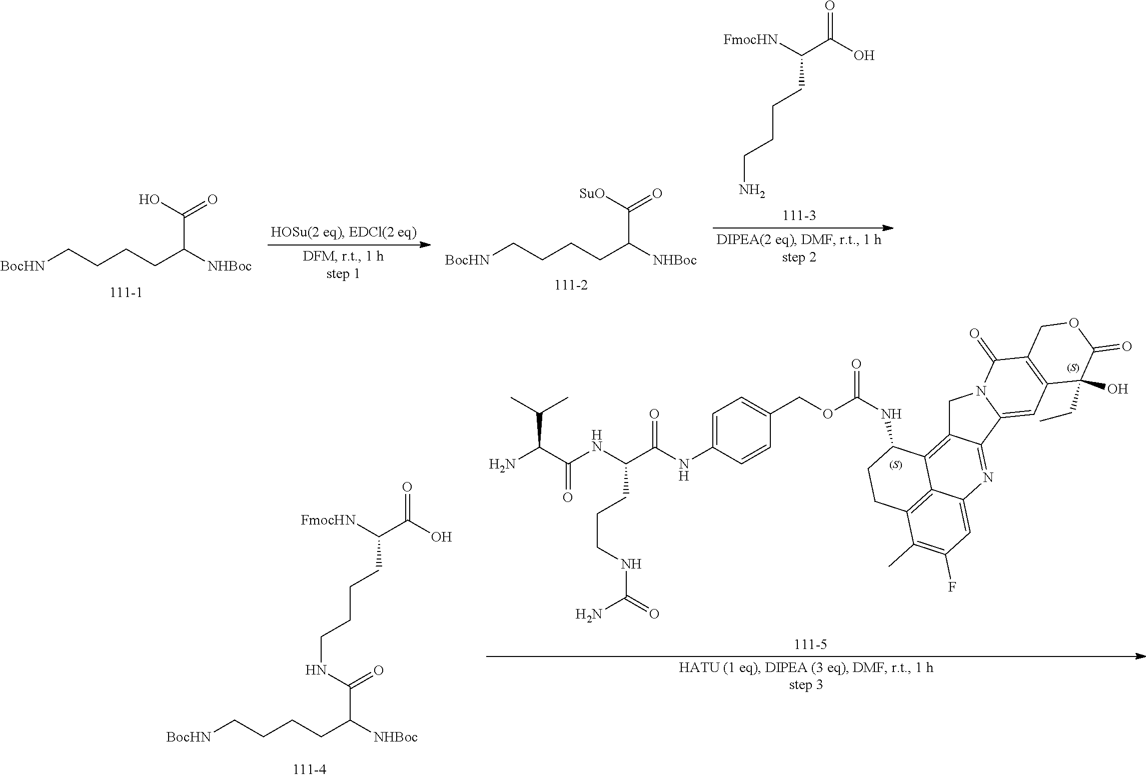

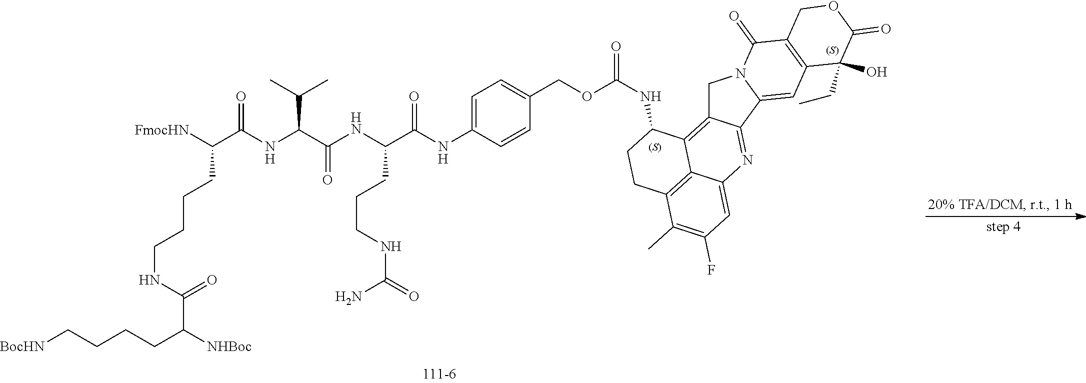

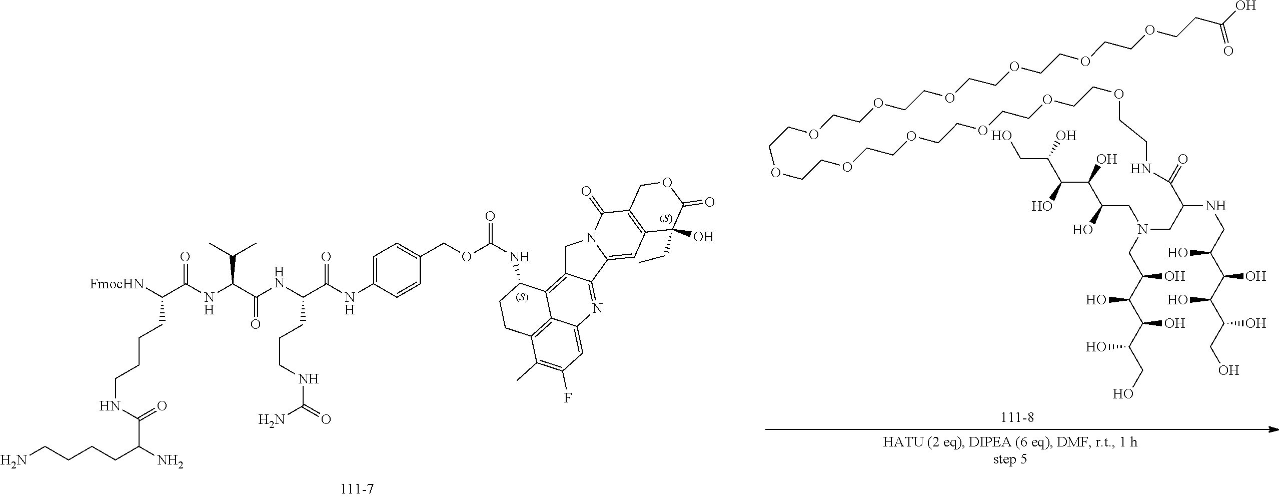

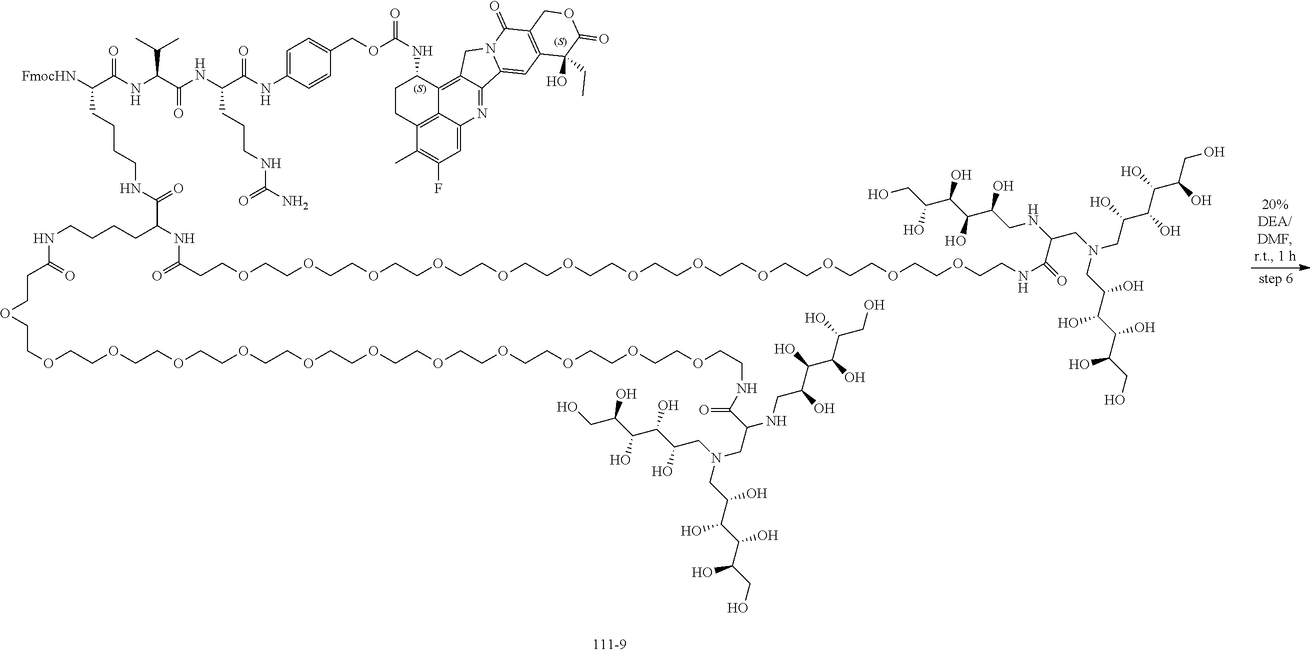

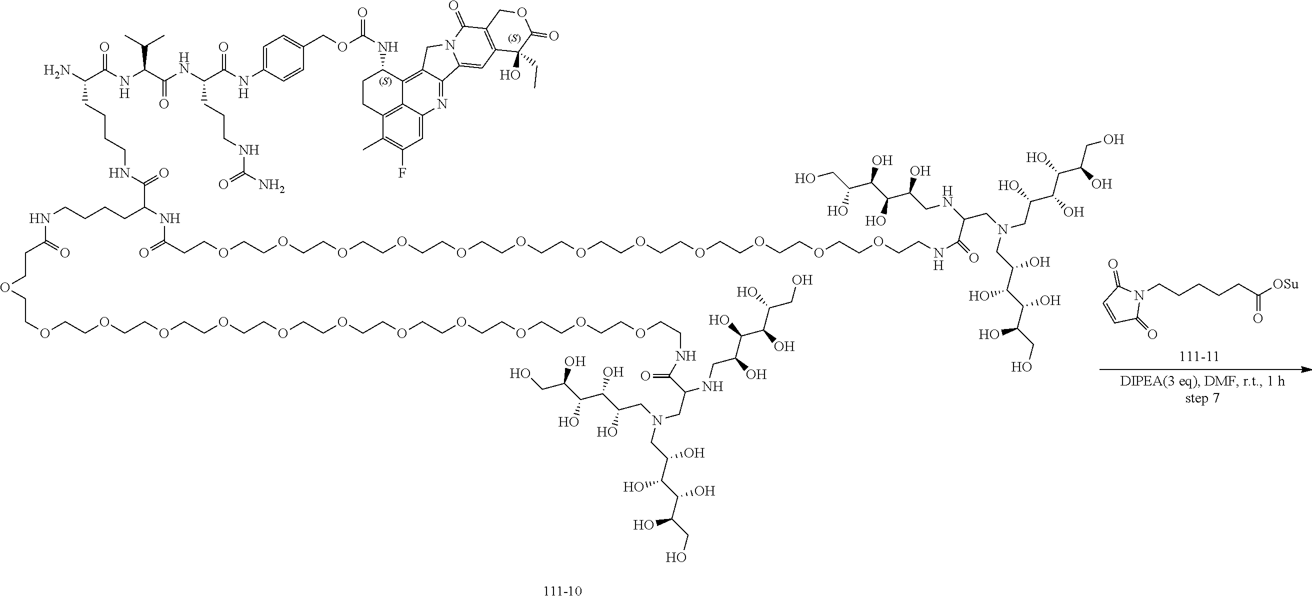

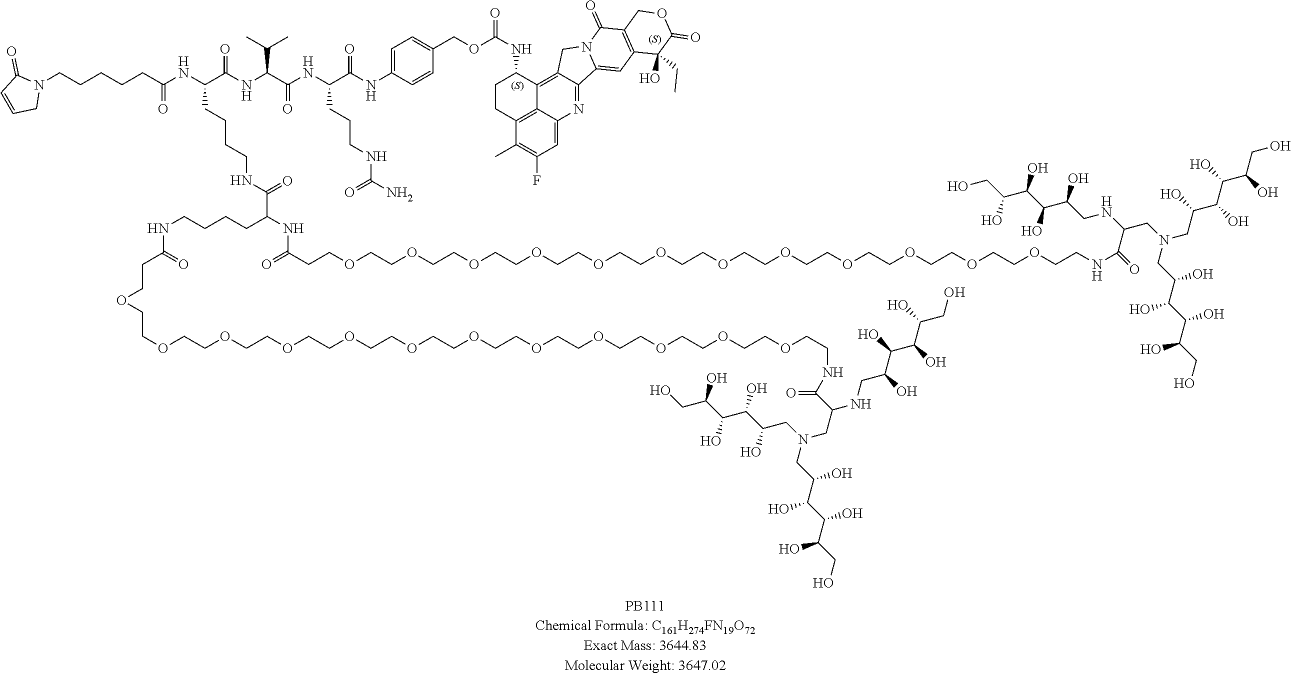

A Drug-Linker containing a PEG unit and a cleavable linker attached to exatecan (PB111 or LD111) was prepared as follows:

### Step 1

A solution of compound 111-1 (5 g, 14.433 mmol) and HOSu (3.3 g, 28.673 mmol) in anhydrous DCM (60 mL) was stirred at room temperature, then EDCl (5.5 g, 28.690 mmol) was added. The resulting solution was stirred for another 1 hr at r.t. until LCMS indicated all starting amine was consumed and the desired product was detected. The resulting solution was washed with water, the organic layer was collected, and then water phase was extracted with DCM (50 mL*2). The combined organic layer was dried over sodium sulfate and filtered, concentrated to dryness to yield compound 111-2 (4.8 g, 10.823 mmol, 75.00%) as a white solid, which was used as such in the next step. purity=85%-90%.

### Step 2

A solution of compound 111-2 (4.8 g, 10.823 mmol) and compound 111-3 (3.99 g, 10.829 mmol) in anhydrous DMF (20 mL) was stirred at room temperature, then DIPEA (2.8 g, 21.665 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 111-4 (5.1 g, 7.319 mmol, 67.64%) as a white solid. purity=90%-95%.

### Step 3

A solution of compound 111-4 (0.83 g, 1.191 mmol), compound 111-5 (1 g, 1.189 mmol) and HATU (0.45 g, 1.183 mmol) in anhydrous DMF (10 mL) was stirred at room temperature for 5 min, then DIPEA (0.46 g, 3.559 mmol) was added. The resulting solution was stirred for another 1 hr at r.t. until LCMS indicated complete reaction. The reaction solution was purified directly by reverse phase liquid chromatography to yield compound 111-6 (1.1 g, 0.724 mmol, 61.11%) as a white solid. purity=90%-95%.

### Step 4

A solution of compound 111-6 (1.1 g, 0.724 mmol) and TFA (2 mL) in anhydrous DCM (8 mL) was stirred at room temperature for 1 hr until LCMS indicated all starting amine was consumed and the desired product was detected. Then the solution was concentrated to dryness to yield compound 111-7 (0.9 g, 0.682 mmol, 94.24%) as a yellow oil, which was used as such in the next step. purity=85%-90%.

### Step 5

A solution of compound 111-7 (150 mg, 0.114 mmol), compound 111-8 (272 mg, 0.227 mmol) and HATU (87 mg, 0.229 mmol) in anhydrous DMF (5 mL) was stirred at room temperature for 5 min, then DIPEA (89 mg, 0.689 mmol) was added. The resulting solution was stirred for another 1 hr at r.t. until LCMS indicated complete reaction. The reaction solution was purified directly by reverse phase liquid chromatography to yield compound 111-9 (110 mg, 0.030 mmol, 26.38%) as a white solid. purity=90%-95%.

### Step 6

A solution of compound 111-9 (110 mg, 0.030 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 111-10 (75 mg, 0.022 mmol, 72.82%) as a colorless oil, which was used as such in the next step. purity=90%-95%.

### Step 7

A solution of compound 111-10 (75 mg, 0.022 mmol) and DIPEA (5.6 mg, 0.043 mmol) in anhydrous DMF (2 mL) was stirred at room temperature for 5 min, then a solution of compound 111-11 (10 mg, 0.032 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB111 (22 mg, 0.006 mmol, 27.78%) as a white solid. LCMS, m/z=912.79(M/4+H)⁺.

### Example 43: Preparation of a Drug-Linker Containing a PEG Unit and a Cleavable Linker Attached to MMAE (PB112)

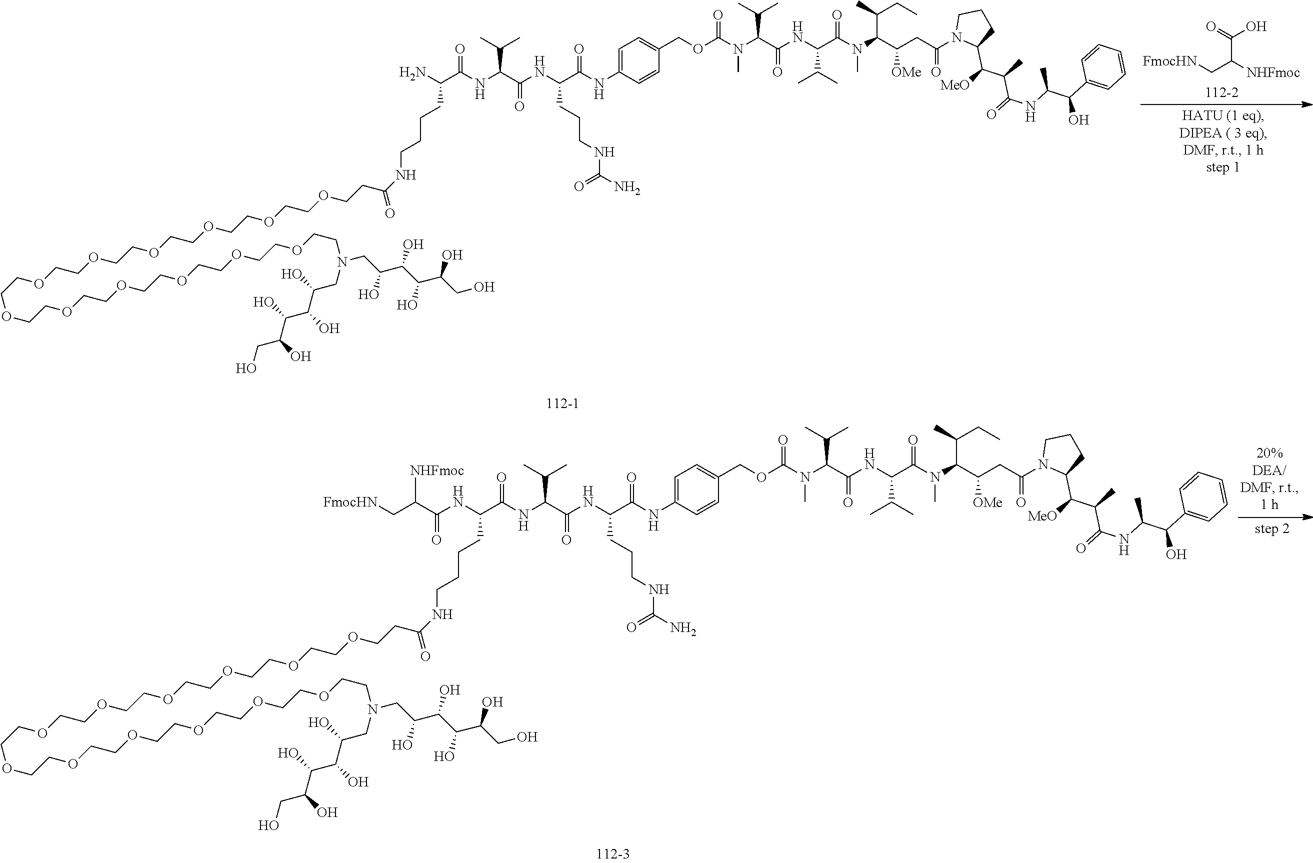

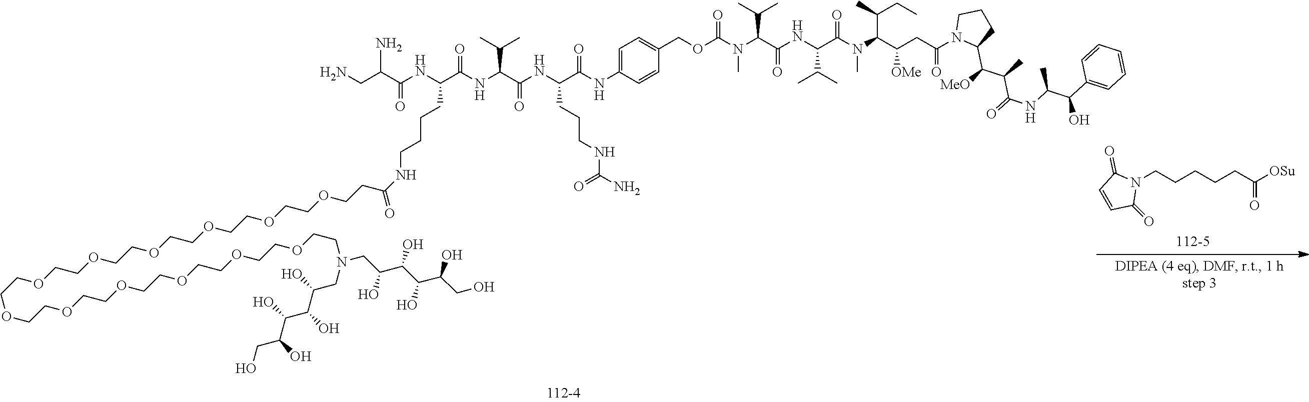

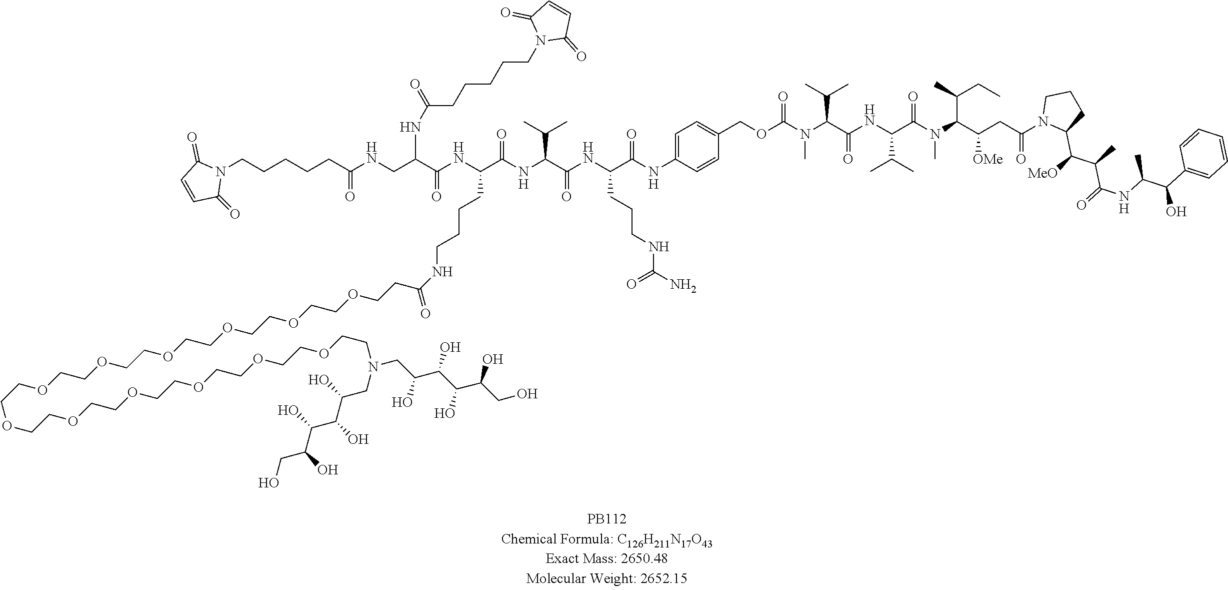 [SMILES: CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)NC(=O)C(CNC(=O)CCCCCN2C(=O)C=CC2=O)NC(=O)CCCCCN2C(=O)C=CC2=O)C(C)C)cc1)C(C)C]

A Drug-Linker containing a PEG unit and a cleavable linker attached to MMAE (PB112) was prepared as follows:

### Step 1

A solution of compound 112-1 (100 mg, 0.046 mmol), compound 112-2 (25.2 mg, 0.046 mmol) and HATU (17.5 mg, 0.046 mmol) in anhydrous DMF (3 mL) was stirred at room temperature for 5 min, then DIPEA (17.8 mg, 0.138 mmol) was added. The resulting solution was stirred for another 1 hr at r.t. until LCMS indicated complete reaction. The reaction solution was purified directly by reverse phase liquid chromatography to yield compound 112-3 (85 mg, 0.031 mmol, 68.55%) as a white solid. purity=90%-95%.

### Step 2

A solution of compound 112-3 (85 mg, 0.031 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 112-4 (65 mg, 0.029 mmol, 91.47%) as a colorless oil, which was used as such in the next step. purity=90%-95%.

### Step 3

A solution of compound 112-4 (65 mg, 0.029 mmol) and DIPEA (14.8 mg, 0.116 mmol) in anhydrous DMF (2 mL) was stirred at room temperature for 5 min, then a solution of compound 112-5 (26.5 mg, 0.087 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB112 (11 mg, 0.004 mmol, 14.47%) as a white solid. LCMS, m/z=885.71 (M/3+H)⁺.

### Example 44: Preparation of a Drug-Linker Containing a PEG Unit and a Cleavable Linker Attached to MMAE (PB113)

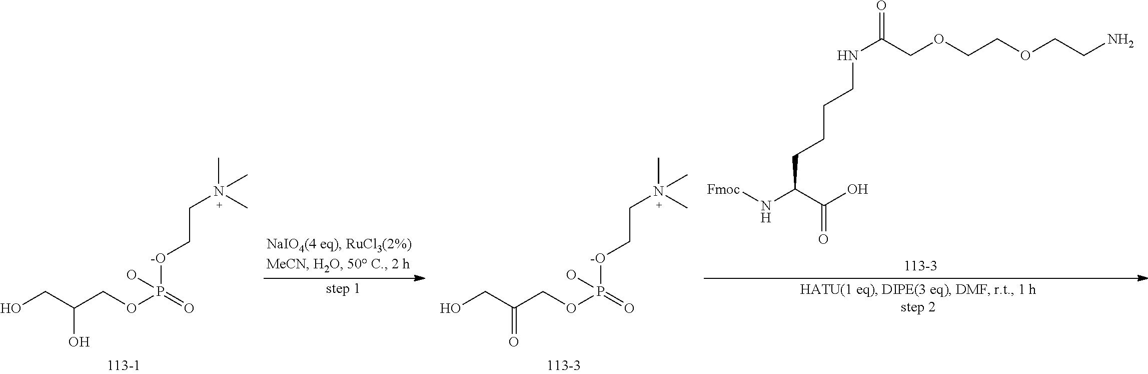

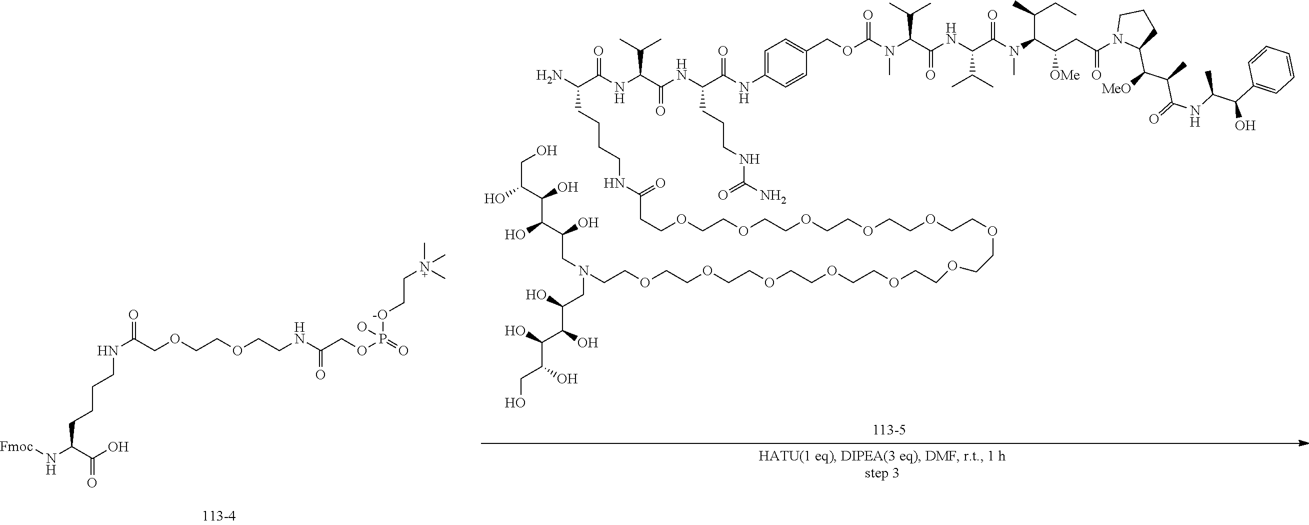

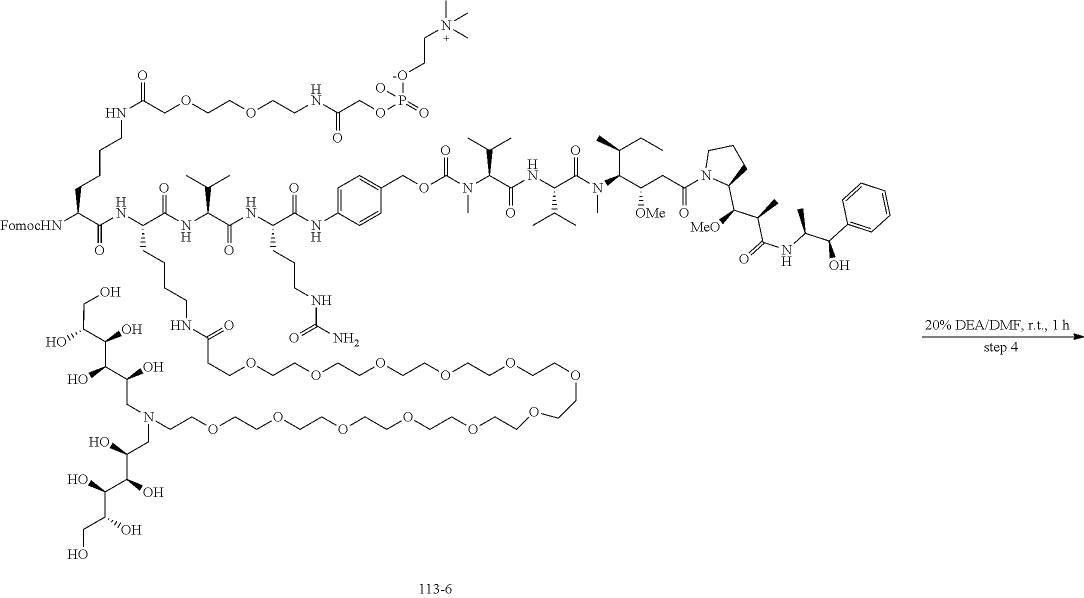

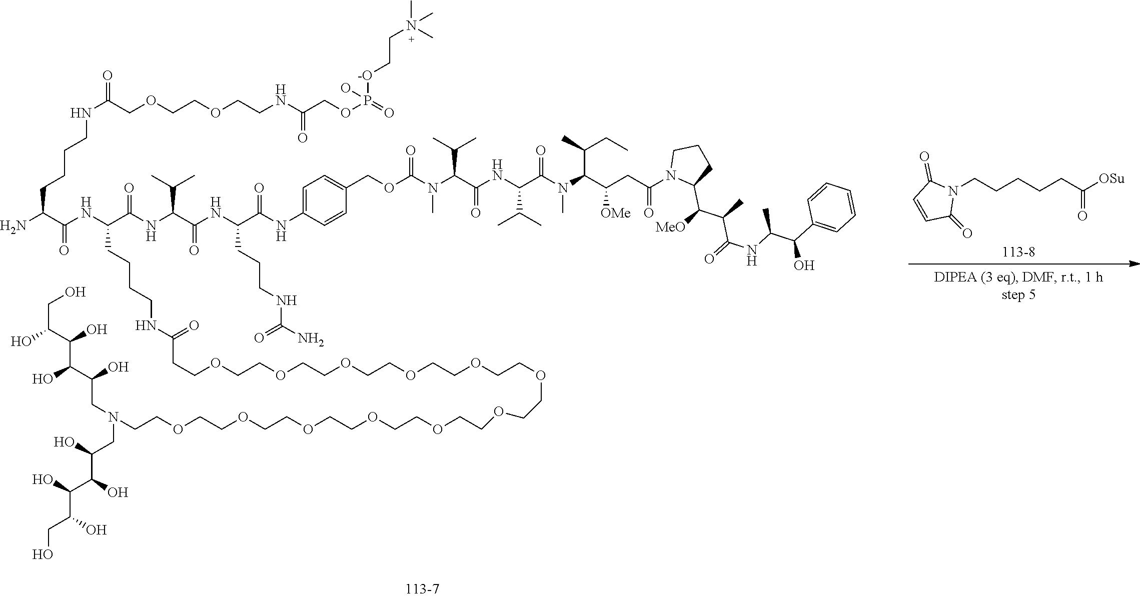

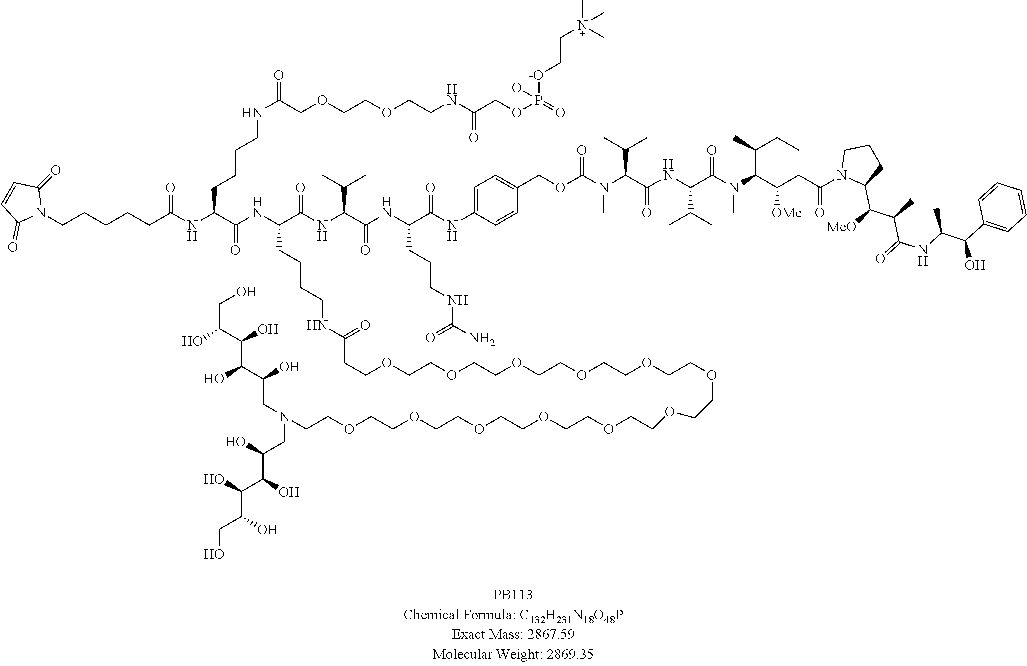

A Drug-Linker containing a PEG unit and a cleavable linker attached to MMAE (PB113) was prepared as follows:

### Step 1

A solution of compound 113-1 (20 g, 0.078 mol) and RuCl₃ (0.4 g, 2%) in MeCN (100 mL) and H₂O (200 mL) was stirred at room temperature for 5 min, then NalO₄ (66.52 g, 0.311 mol) was added in several portions. The resulting solution was stirred for another 2 hr at 50° C. until LCMS indicated all starting amine was consumed and the desired product was detected. Methanol (20 mL) was added, the solution was evaporated and the residue was washed in acetonitrile (100 ml) at 5° C. for 30 minutes. The solution was filtered and the filtrate was concentrated to dryness to yield compound 113-2 (9.5 g, 0.039 mol, 50.67%) as a white solid. purity=50%-60%.

### Step 2

A solution of compound 113-2 (5 g, 0.021 mol), compound 113-3 (1 g, 1.95 mmol) and HATU (0.74 g, 1.95 mmol) in anhydrous DMF (20 mL) was stirred at room temperature for 5 min, then DIPEA (0.75 g, 5.85 mmol) was added. The resulting solution was stirred for another 1 hr at r.t. until LCMS indicated complete reaction. The reaction solution was purified directly by reverse phase liquid chromatography to yield compound 113-4 (410 mg, 0.556 mmol, 28.67%) as a white solid. purity=90%-95%.

### Step 3

A solution of compound 113-4 (20.6 mg, 0.028 mol), compound 113-5 (60 mg, 0.028 mmol) and HATU (10.5 mg, 0.028 mmol) in anhydrous DMF (2 mL) was stirred at room temperature for 5 min, then DIPEA (10.7 mg, 0.084 mmol) was added. The resulting solution was stirred for another 1 hr at r.t. until LCMS indicated complete reaction. The reaction solution was purified directly by reverse phase liquid chromatography to yield compound 113-6 (41 mg, 0.014 mmol, 51.38%) as a white solid. purity=90%-95%.

### Step 4

A solution of compound 113-6 (41 mg, 0.014 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 113-7 (31 mg, 0.012 mmol, 81.79%) as a yellow oil, which was used as such in the next step. purity=90%-95%.

### Step 5

A solution of compound 113-7 (31 mg, 0.012 mmol) and DIPEA (4.5 mg, 0.035 mmol) in anhydrous DMF (1 mL) was stirred at room temperature for 5 min, then a solution of compound 113-8 (5.4 mg, 0.017 mmol) in anhydrous DMF (1 mL) was added dropwise by syringe over 2 min. The resulting solution was stirred for another 1 hr at r.t. until LCMS indicated all starting amine was consumed and the mass of desired product was detected. The resulting solution was neutralized with formic acid to adjust the pH to 6-7. Then the reaction solution was purified by Prep-HPLC to yield PB113 (21 mg, 0.007 mmol, 63.25%) as a white solid. LCMS, m/z=957.42 (M/3+H)⁺.

### Example 45: Preparation of a Drug-Linker Attached to Exatecan (PB114)

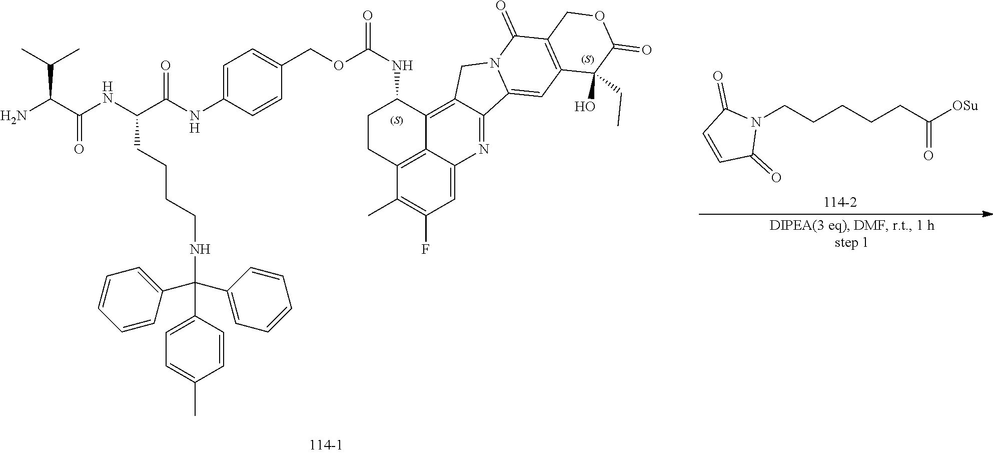

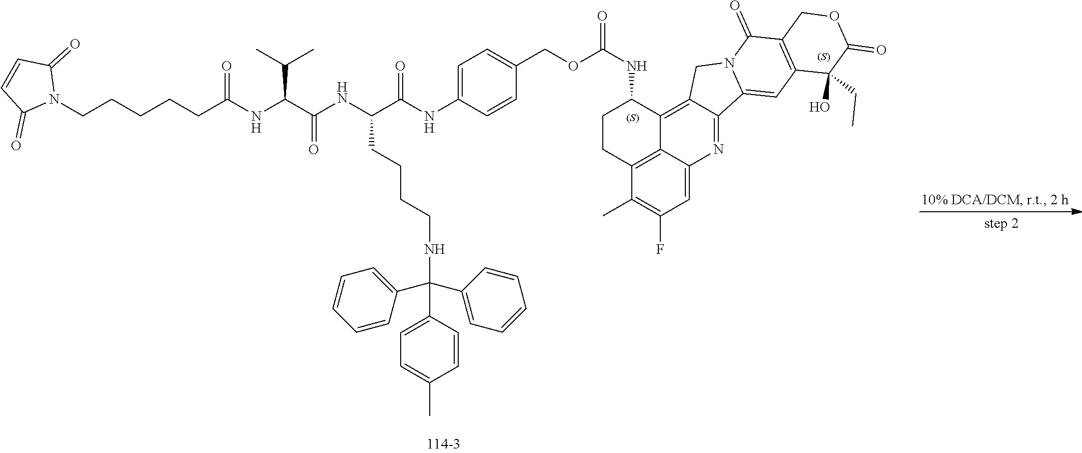

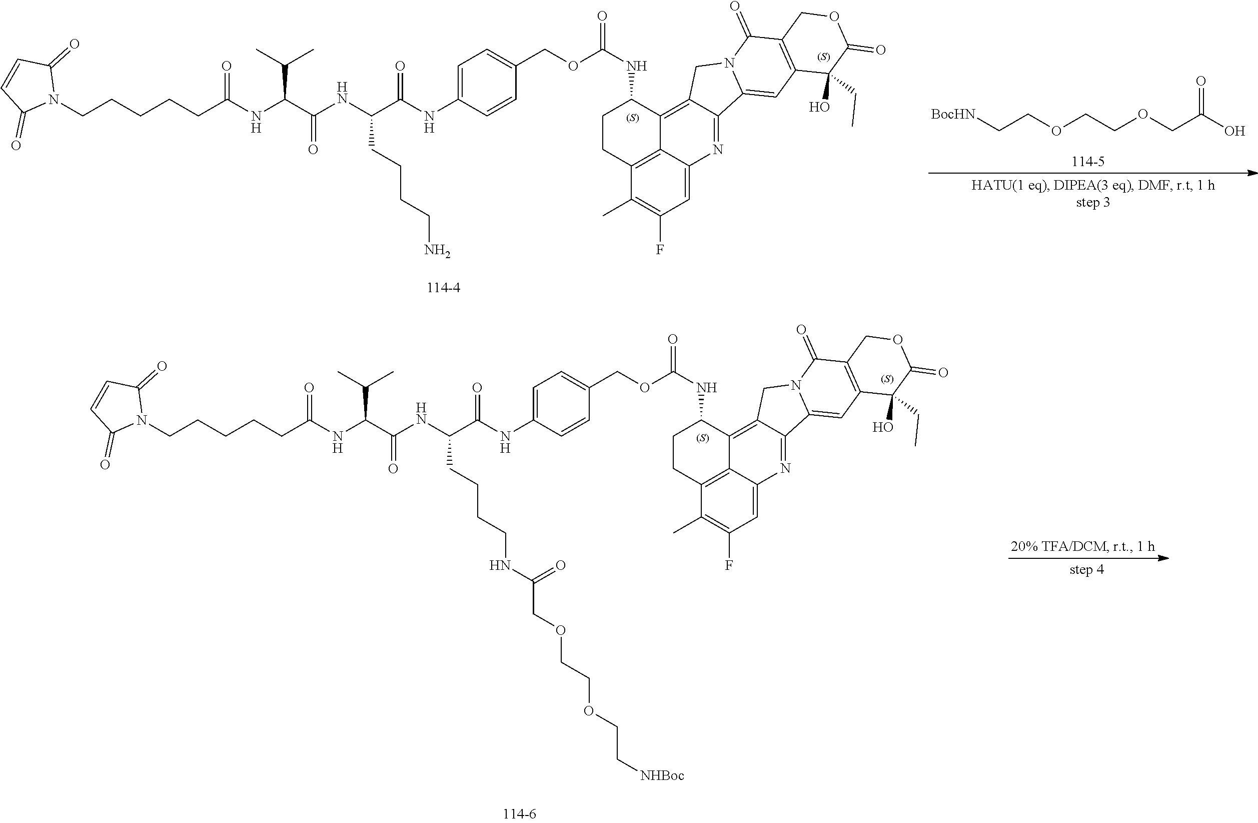

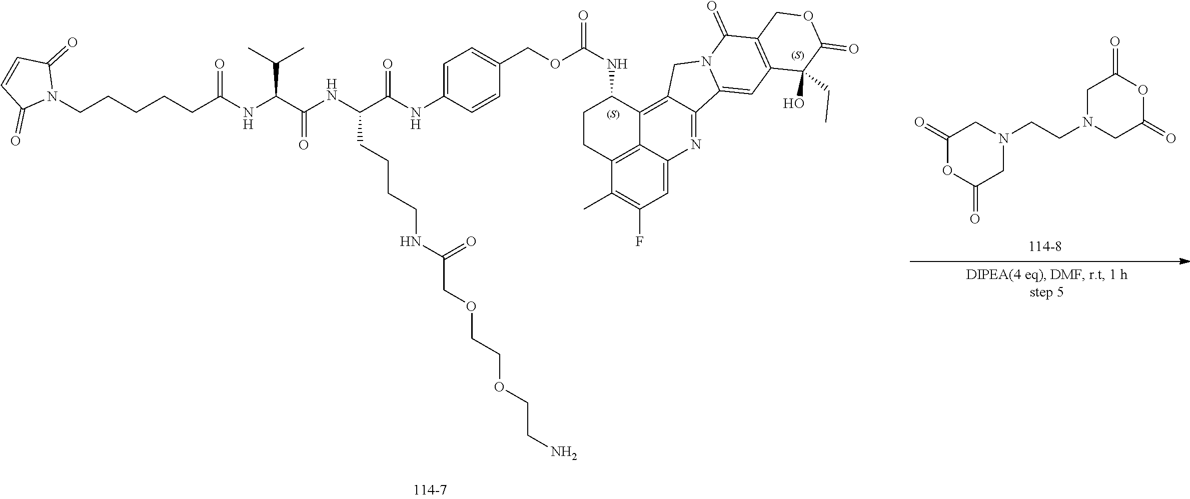

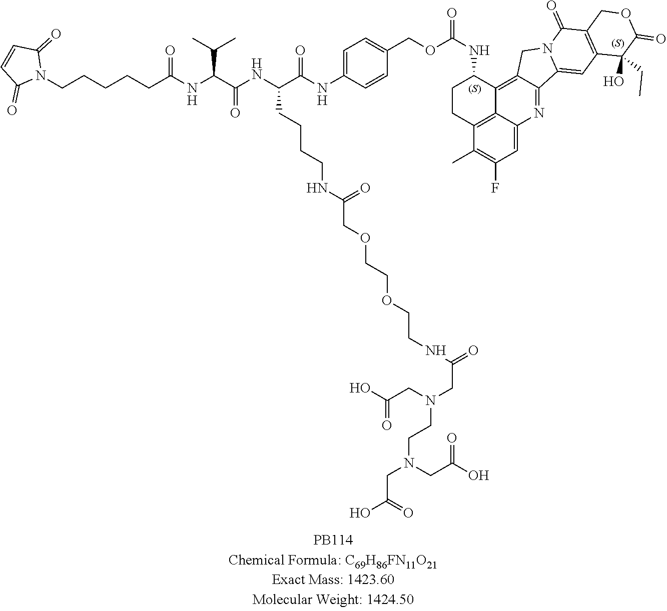

A Drug-Linker attached to exatecan (PB114) was prepared as follows:

### Step 1

A solution of compound 114-1 (5.8 g, 5.431 mmol) and DIPEA (2.1 g, 16.249 mmol) in anhydrous DMF (30 mL) was stirred at room temperature for 5 min, then a solution of compound 114-2 (2.5 g, 8.109 mmol) in anhydrous DMF (10 mL) was added dropwise by syringe over 5 min. The resulting solution was stirred for another 1 hr at r.t. until LCMS indicated all starting amine was consumed and the mass of desired product was detected. Then most of the DMF and DIPEA was evaporated and the residue was washed in acetonitrile (150 ml) at 5° C. for 30 minutes. The solution was filtered and the filter cake was washed with acetonitrile to yield compound 114-3 (5.2 g, 4.122 mmol, 75.91%) as a yellow solid. purity=85%-95%.

### Step 2

A solution of compound 114-3 (5.2 g, 4.122 mmol) and DCA (10 mL) in anhydrous DCM (90 mL) was stirred at room temperature for 2 hr until LCMS indicated all starting amine was consumed and the desired product was detected. The reaction solution was concentrated to dryness and purified directly by reverse phase liquid chromatography to yield compound 114-4 (2.5 g, 2.488 mmol, 60.24%) as a yellow solid. purity=85%-95%.

### Step 3

A solution of compound 114-4 (500 mg, 0.497 mmol), compound 114-5 (131 mg, 0.498 mmol) and HATU (189 mg, 0.497 mmol) in anhydrous DMF (5 mL) was stirred at room temperature for 5 min, then DIPEA (193 mg, 1.493 mmol) was added. The resulting solution was stirred for another 1 hr at r.t. until LCMS indicated complete reaction. The reaction solution was purified directly by reverse phase liquid chromatography to yield compound 114-6 (160 mg, 0.128 mmol, 25.72%) as a yellow solid. purity=85%-95%.

### Step 4

A solution of compound 114-6 (160 mg, 0.128 mmol) and TFA (1 mL) in anhydrous DCM (4 mL) was stirred at room temperature for 1 hr until LCMS indicated all starting amine was consumed and the desired product was detected. The reaction solution was purified directly by reverse phase liquid chromatography to yield compound 114-7 (50 mg, 0.043 mmol, 33.97%) as a yellow solid. purity=85%-95%.

### Step 5

A solution of compound 114-8 (16.7 mg, 0.065 mmol) and DIPEA (22.5 mg, 0.174 mmol) in anhydrous DMF (2 mL) was stirred at room temperature for 10 min, then a solution of compound 114-7 (50 mg, 0.043 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB114 (20 mg, 0.014 mmol, 32.31%) as a yellow solid. LCMS, m/z=713.19 (M/2+H)⁺.

### Example 46: Preparation of a Drug-Linker Attached to Exatecan (PB115; SEQ ID NO: 63). Compound 115-6 is Disclosed as SEQ ID NO: 67, Compound 115-7 is Disclosed as SEQ ID NO: 66, Compound 115-9 is Disclosed as SEQ ID NO: 64, and Compound 115-10 is Disclosed as SEQ ID NO: 65

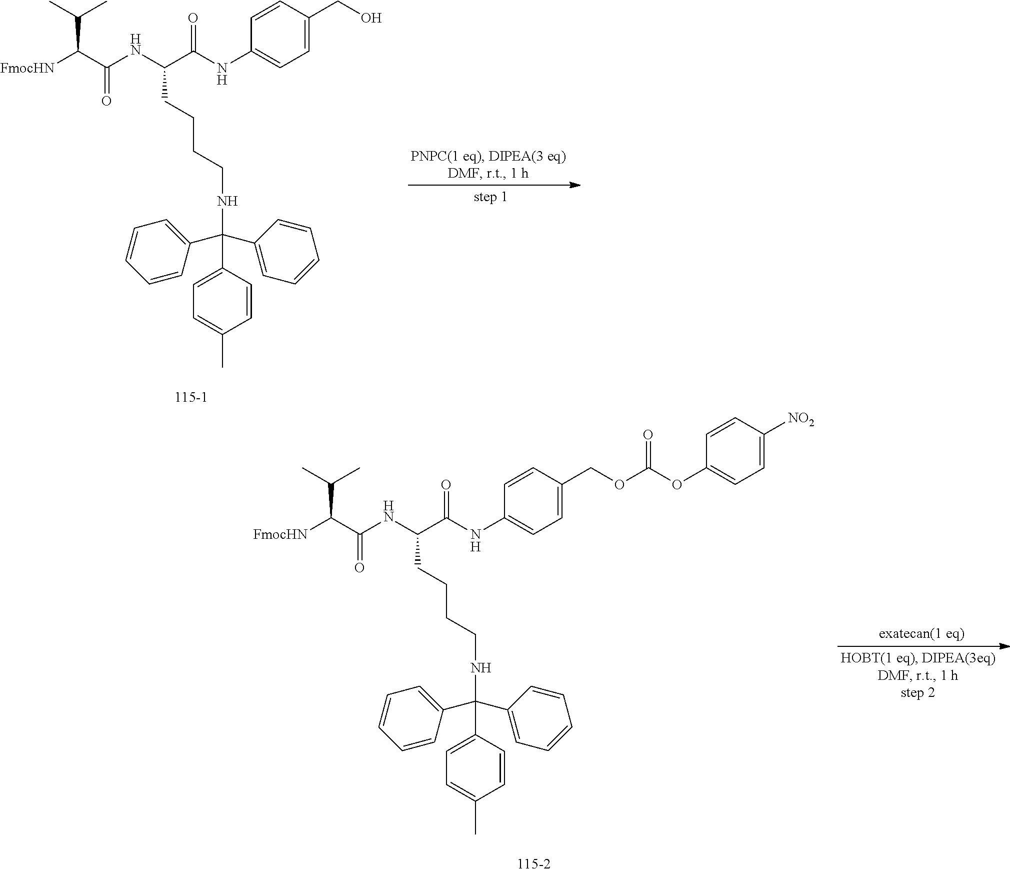

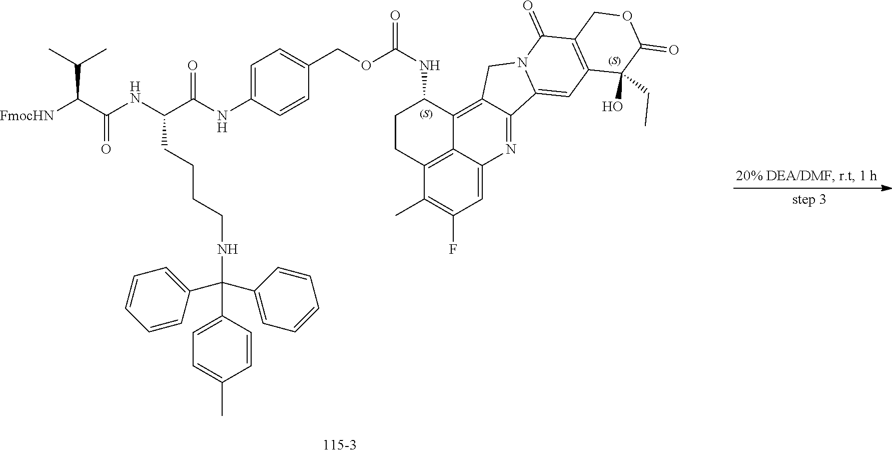

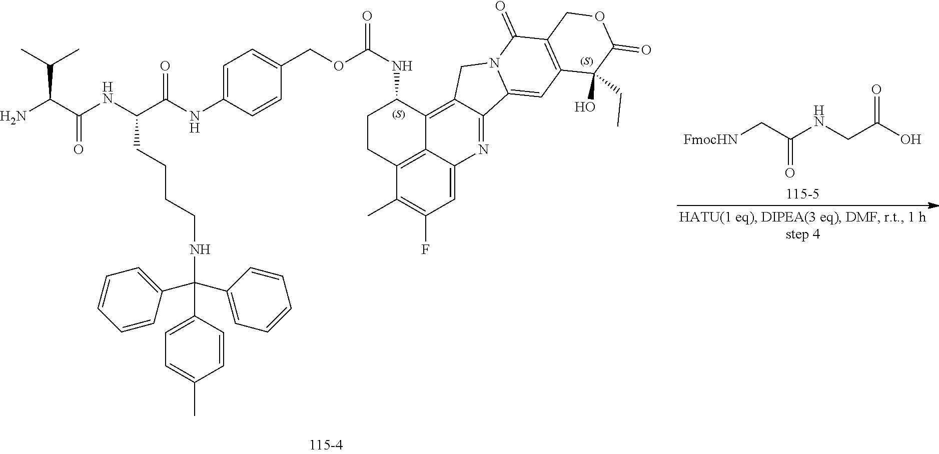

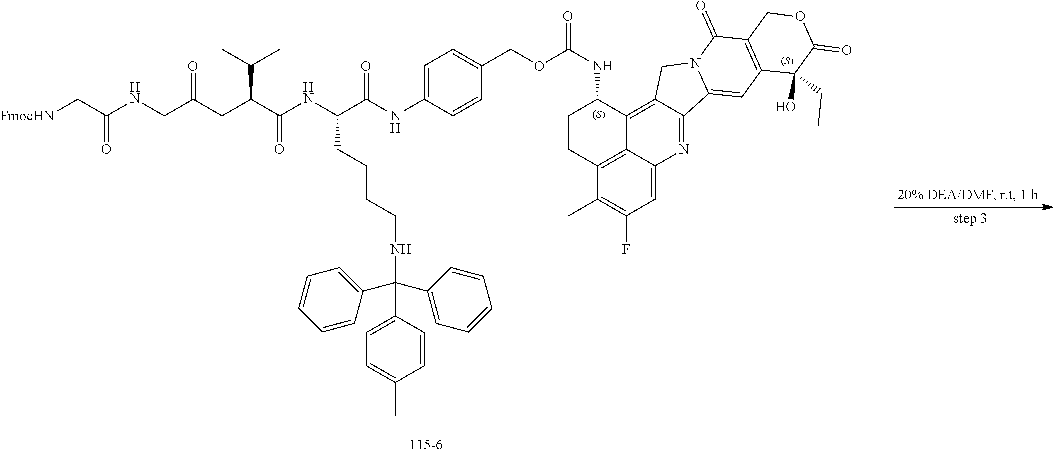

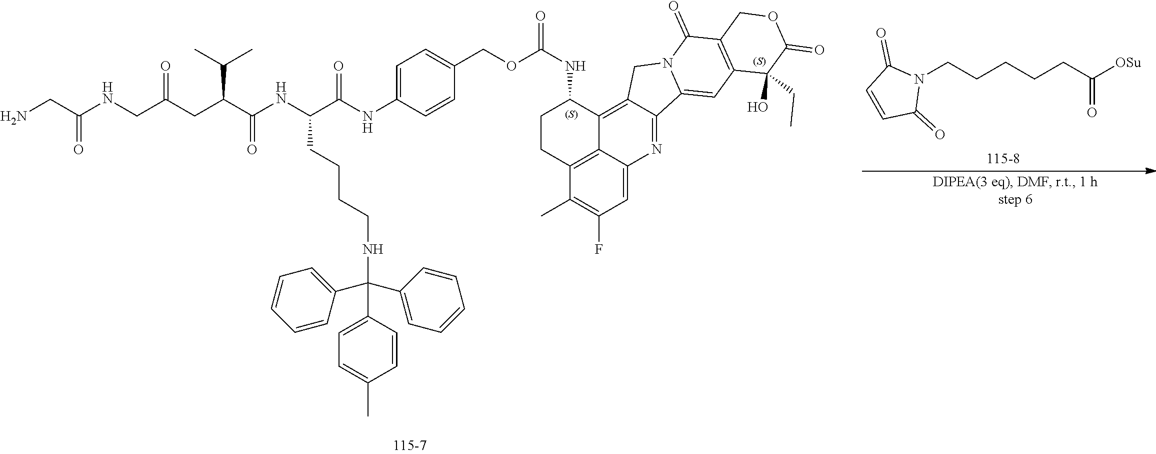

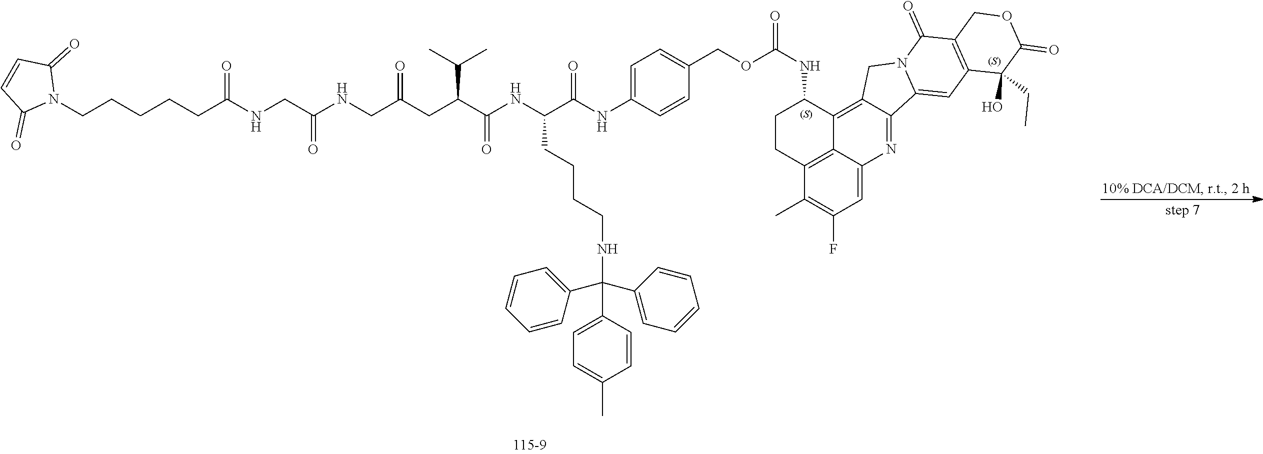

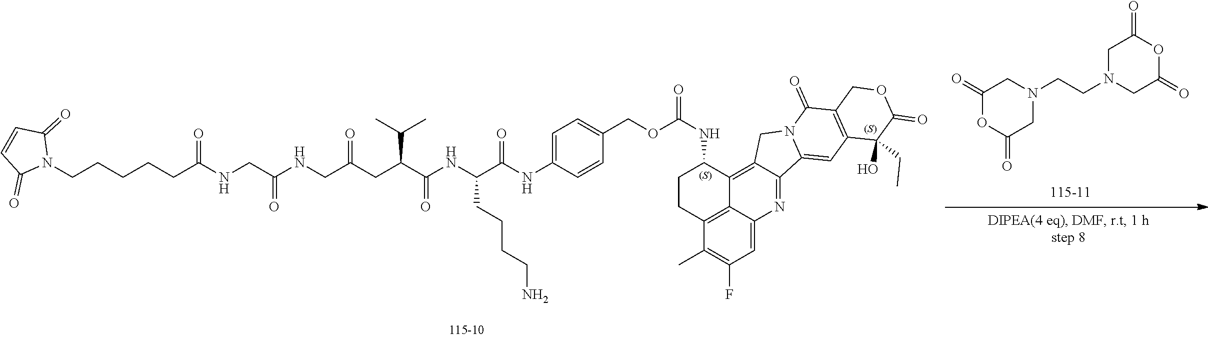

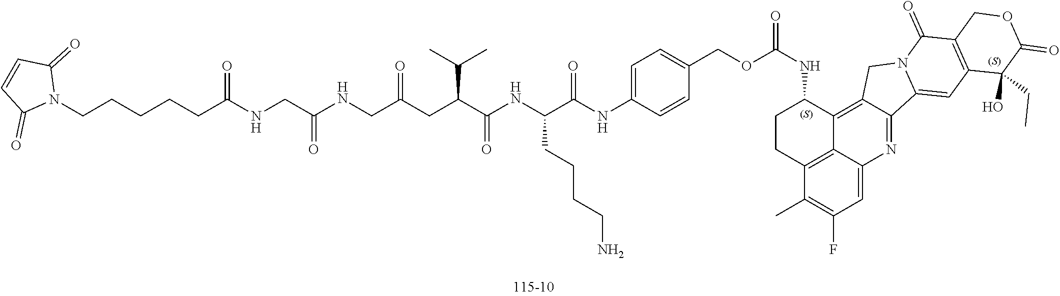

A Drug-Linker attached to exatecan (PB115) was prepared as follows:

### Step 1

A solution of compound 115-1 (10 g, 0.012 mol) and DIPEA (4.6 g, 0.036 mol) in anhydrous DMF (60 mL) was stirred at room temperature for 5 min, then PNPC (3.67 g, 0.012 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (200 ml) at 5° C. for 30 minutes. The solution was filtered and the filter cake was washed with acetonitrile to yield compound 115-2 (8.4 g, 8.449 mmol, 70.06%) as a gray solid. purity=85%-95%.

### Step 2

A solution of compound 115-2 (8.4 g, 8.449 mmol), exatecan (4.5 g, 8.449 mmol) and HOBT (1.1 g, 8.449 mmol) in anhydrous DMF (50 mL) was stirred at room temperature, then DIPEA (3.3 g, 25.347 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (100 ml) at 5° C. for 30 minutes. The solution was filtered and the filter cake was washed with acetonitrile to yield compound 115-3 (9.3 g, 7.207 mmol, 85.32%) as a gray solid. purity=85%-95%.

### Step 3

A solution of compound 115-3 (9.3 g, 7.207 mmol) and DEA (10 mL) in anhydrous DMF (40 mL) was stirred at room temperature for 1 hr until LCMS indicated all starting amine was consumed and the desired product was detected. Then most of the DMF and DEA was evaporated and the residue was washed in acetonitrile (200 ml) at 5° C. for 30 minutes. The solution was filtered and the filter cake was washed with acetonitrile to yield compound 115-4 (5.8 g, 5.429 mmol, 75.32%) as a gray solid. purity=85%-95%.

### Step 4

A solution of compound 115-4 (1.6 g, 1.498 mmol), compound 115-5 (0.53 g, 1.498 mmol) and HATU (0.57 g, 1.498 mmol) in anhydrous DMF (10 mL) was stirred at room temperature for 5 min, then DIPEA (0.58 g, 4.494 mmol) was added. The resulting solution was stirred for another 1 hr at r.t. until LCMS indicated complete reaction. Then most of the DMF and DIPEA was evaporated and the residue was washed in acetonitrile (50 ml) at 5° C. for 30 minutes. The solution was filtered and the filter cake was washed with acetonitrile to yield compound 115-6 (1.7 g, 1.210 mmol, 80.95%) as a gray solid. purity=85%-95%.

### Step 5

A solution of compound 115-6 (1.7 g, 1.210 mmol) and DEA (2 mL) in anhydrous DMF (8 mL) was stirred at room temperature for 1 hr until LCMS indicated all starting amine was consumed and the desired product was detected. Then most of the DMF and DEA was evaporated and the residue was washed in acetonitrile (50 ml) at 5° C. for 30 minutes. The solution was filtered and the filter cake was washed with acetonitrile to yield compound 115-7 (1.1 g, 0.930 mmol, 76.92%) as a gray solid. purity=85%-95%.

### Step 6

A solution of compound 115-7 (1.1 g, 0.930 mmol) and DIPEA (0.36 g, 2.791 mmol) in anhydrous DMF (5 mL) was stirred at room temperature for 5 min, then a solution of compound 115-8 (0.43 g, 1.396 mmol) in anhydrous DMF (5 mL) was added dropwise by syringe over 2 min. The resulting solution was stirred for another 1 hr at r.t. until LCMS indicated all starting amine was consumed and the mass of desired product was detected. Then most of the DMF and DIPEA was evaporated and the residue was washed in acetonitrile (30 ml) at 5° C. for 30 minutes. The solution was filtered and the filter cake was washed with acetonitrile to yield compound 115-9 (1.1 g, 0.800 mmol, 85.94%) as a gray solid. purity=80%-90%.

### Step 7

A solution of compound 115-9 (1.1 g, 0.800 mmol) and DCA (2 mL) in anhydrous DCM (18 mL) was stirred at room temperature for 2 hr until LCMS indicated all starting amine was consumed and the desired product was detected. The reaction solution was concentrated to dryness and purified directly by reverse phase liquid chromatography to give 115-10 (310 mg, 0.277 mmol, 34.64%) as a white solid. purity=85%-95%.

### Step 8

A solution of compound 115-10 (141.9 mg, 0.554 mmol) and DIPEA (143.2 mg, 1.108 mmol) in anhydrous DMF (2 mL) was stirred at room temperature for 20 min, then a solution of 115-11 (310 mg, 0.277 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB115 (50 mg, 0.036 mmol, 12.95%) as a white solid. LCMS, m/z=697.66 (M/2+H)⁺.

### Example 47: Preparation of a Drug-Linker Attached to Palbociclib (PB116)

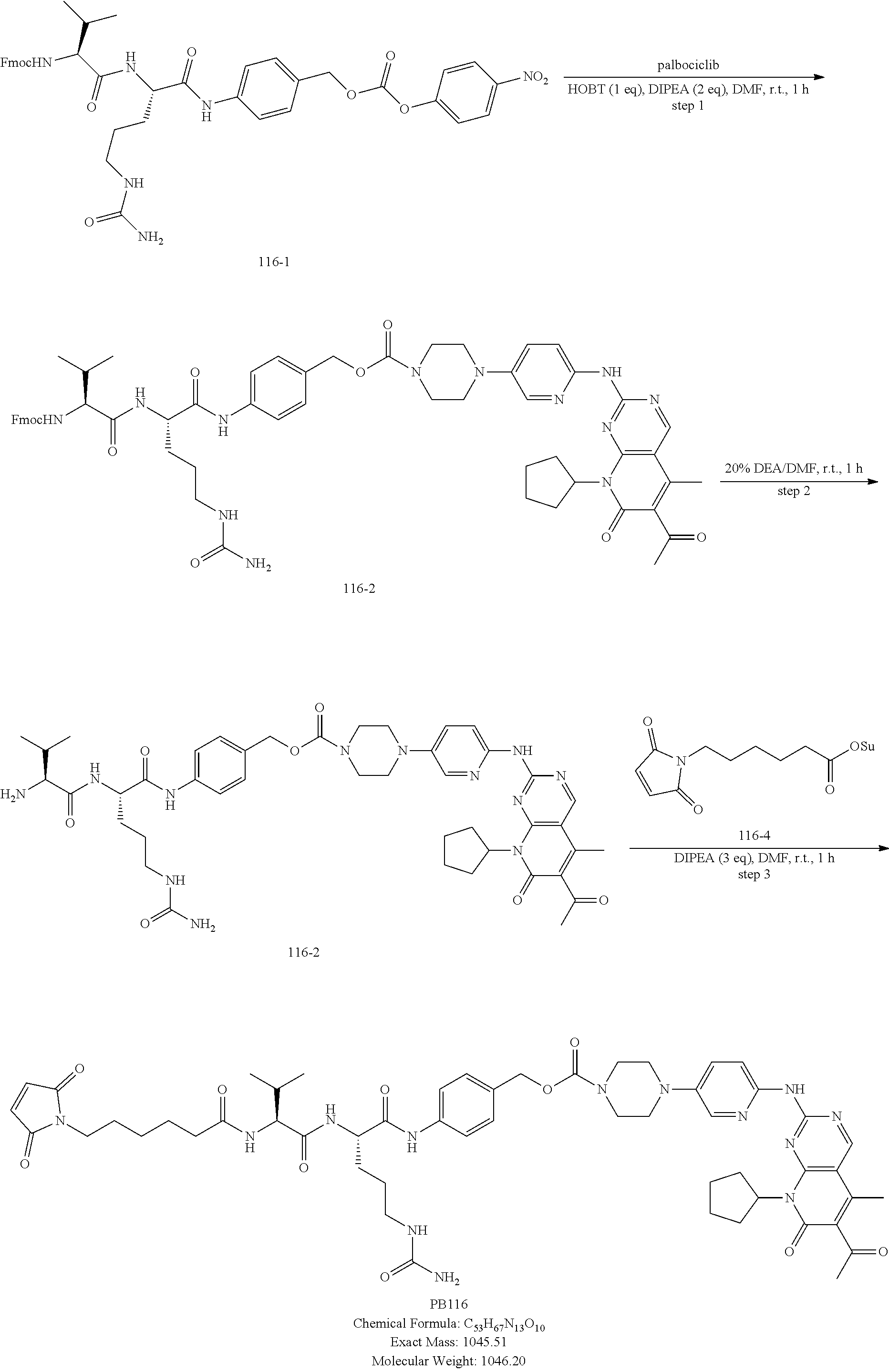

A Drug-Linker attached to palbociclib (PB116) was prepared as follows:

### Step 1

A solution of compound 116-1 (685.4 mg, 0.894 mmol), palbociclib (400 mg, 0.894 mmol) and HOBT (120.8 mg, 0.894 mmol) in anhydrous DMF (60 mL) was stirred at room temperature, then DIPEA (231 mg, 1.787 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (20 ml) at 5° C. for 30 minutes. The solution was filtered and the filter cake was washed with acetonitrile to yield compound 116-2 (720 mg, 0.670 mmol, 74.92%) as a yellow solid. purity=90%-95%.

### Step 2

A solution of compound 116-2 (720 mg, 0.670 mmol) and DEA (1 mL) in anhydrous DMF (4 mL) was stirred at room temperature for 1 hr until LCMS indicated all starting amine was consumed and the desired product was detected. Then most of the DMF and DEA was evaporated and the residue was washed in acetonitrile (50 ml) at 5° C. for 30 minutes. The solution was filtered and the filter cake was washed with acetonitrile to yield compound 116-3 (510 mg, 0.598 mmol, 89.32%) as a yellow solid. purity=95%-95%.

### Step 3

A solution of compound 116-3 (300 mg, 0.352 mmol) and DIPEA (136.5 mg, 1.056 mmol) in anhydrous DMF (3 mL) was stirred at room temperature for 5 min, then a solution of compound 116-4 (162.5 mg, 0.527 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield compound PB116 (210 mg, 0.201 mmol, 57.08%) as a yellow solid. LCMS, m/z=1046.59 (M+H)⁺.

### Example 48: Preparation of a Drug-Linker Containing a PEG Unit and a Cleavable Linker Attached to Palbociclib (PB117)

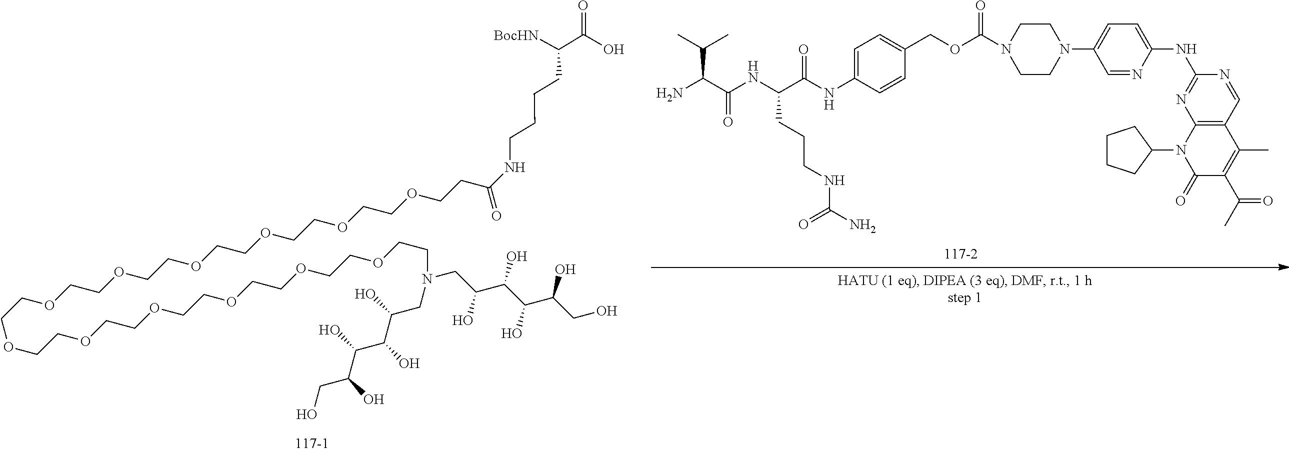 [SMILES: CC(C)(C)OC(=O)N[C@@H](CCCCNC(=O)CCOCCOCCOCCOCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)O ; CC(=O)c1c(C)c2cnc(Nc3ccc(N4CCN(C(=O)OCc5ccc(NC(=O)[C@H](CCCNC(N)=O)NC(=O)[C@@H](N)C(C)C)cc5)CC4)cn3)nc2n(C2CCCC2)c1=O]

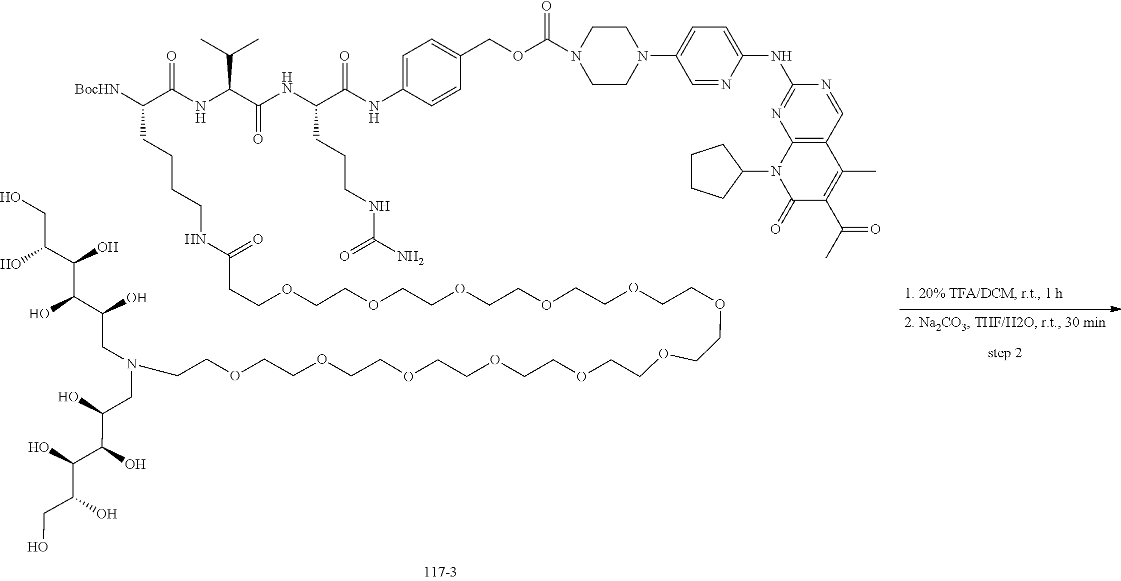 [SMILES: CC(=O)c1c(C)c2cnc(Nc3ccc(N4CCN(C(=O)OCc5ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)OC(C)(C)C)C(C)C)cc5)CC4)cn3)nc2n(C2CCCC2)c1=O]

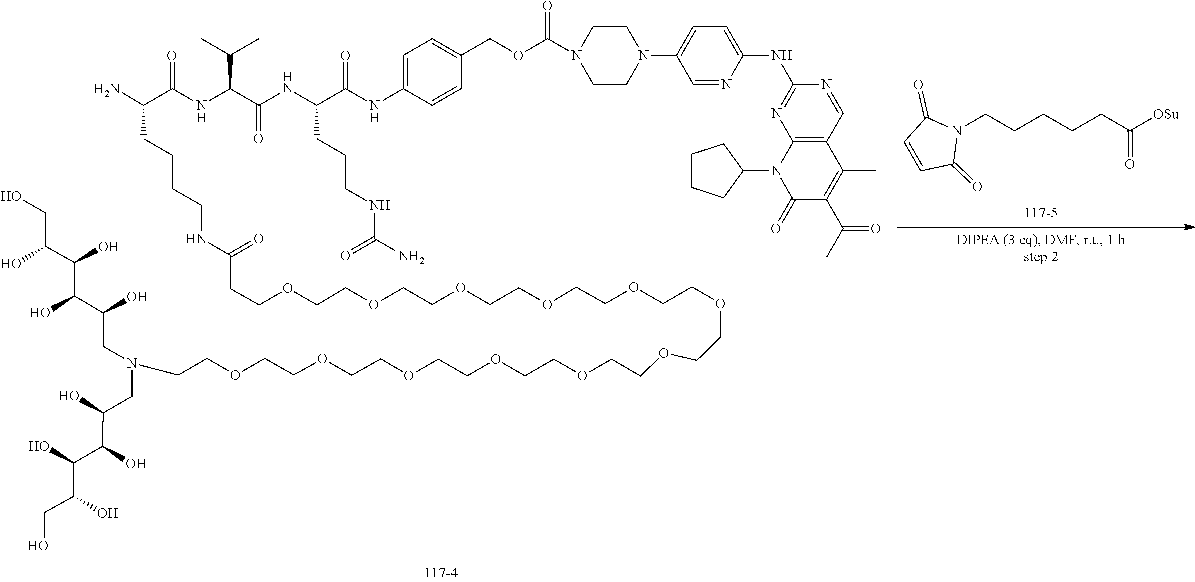

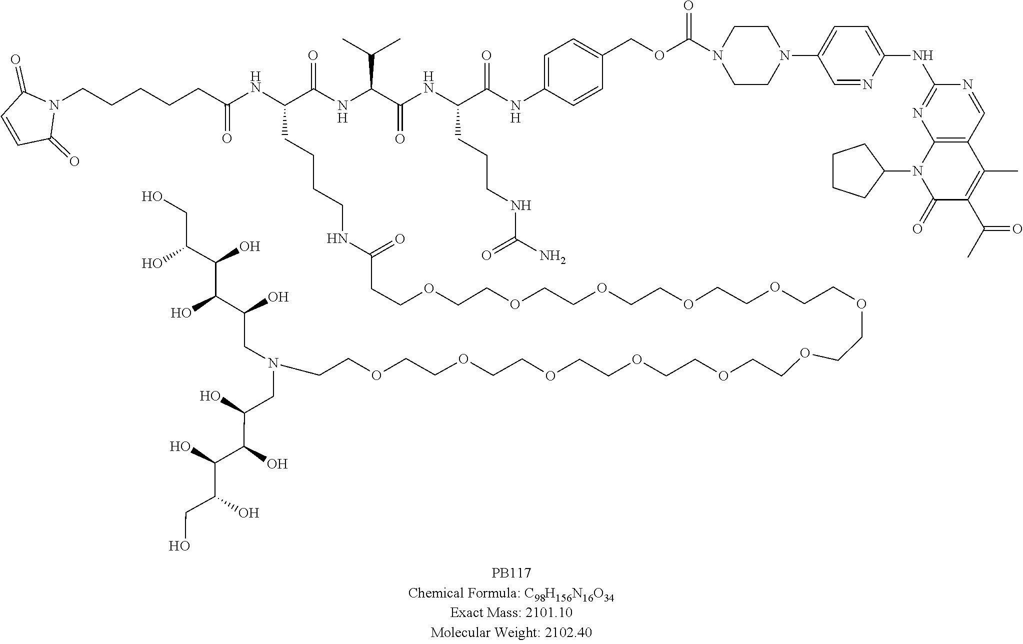 [SMILES: CC(=O)c1c(C)c2cnc(Nc3ccc(N4CCN(C(=O)OCc5ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN6C(=O)C=CC6=O)C(C)C)cc5)CC4)cn3)nc2n(C2CCCC2)c1=O]

A Drug-Linker containing a PEG unit and a cleavable linker attached to palbociclib (PB117) was prepared as follows:

### Step 1

A solution of compound 117-1 (207 mg, 0.176 mmol), compound 117-2 (150 mg, 0.176 mmol) and HATU (66.8 mg, 0.176 mmol) in anhydrous DMF (2 mL) was stirred at room temperature for 5 min, then DIPEA (68.1 mg, 0.527 mmol) was added. The resulting solution was stirred for another 1 hr at r.t. until LCMS indicated complete reaction. The reaction solution was purified directly by reverse phase liquid chromatography to yield compound 117-3 (210 mg, 0.105 mmol, 59.49%) as a yellow solid. purity=90%-95%.

### Step 2

A solution of compound 117-3 (210 mg, 0.105 mmol) and TFA (2 mL) in anhydrous DCM (8 mL) was stirred at room temperature for 1 hr until LCMS indicated all starting amine was consumed and the desired product (m/z=637=1909/3+H) along with sugar-esterificated product (TFA was condensed with hydroxy group in sugar unit, mono-ester with m/z=(1909+96)/3+H=669) were formed. The completed reaction solution was condensed to dryness and then redissolved in THF (4 mL) and water (2 mL), treated with saturated aqueous sodium carbonate solution to adjust the pH to 8-9. The resulting solution was stirred at room temperature for 30 min to achieve complete hydrolysis. The solution was then neutralized with diluted TFA and condensed, the residue was purified by reverse phase liquid chromatography to yield compound 117-4 (120 mg, 0.063 mmol, 60.15%) as a yellow solid. purity=90%-95%.

### Step 3

A solution of compound 117-4 (120 mg, 0.063 mmol) and DIPEA (24.3 mg, 0.189 mmol) in anhydrous DMF (3 mL) was stirred at room temperature for 5 min, then a solution of compound 117-5 (29.0 mg, 0.094 mmol) in anhydrous DMF (2 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB117 (40 mg, 0.019 mmol, 30.30%) as a yellow solid. LCMS, m/z=1052.11 (M/2+H)⁺.

### Example 49: Preparation of a Drug-Linker attached to T7-2 (PB118)

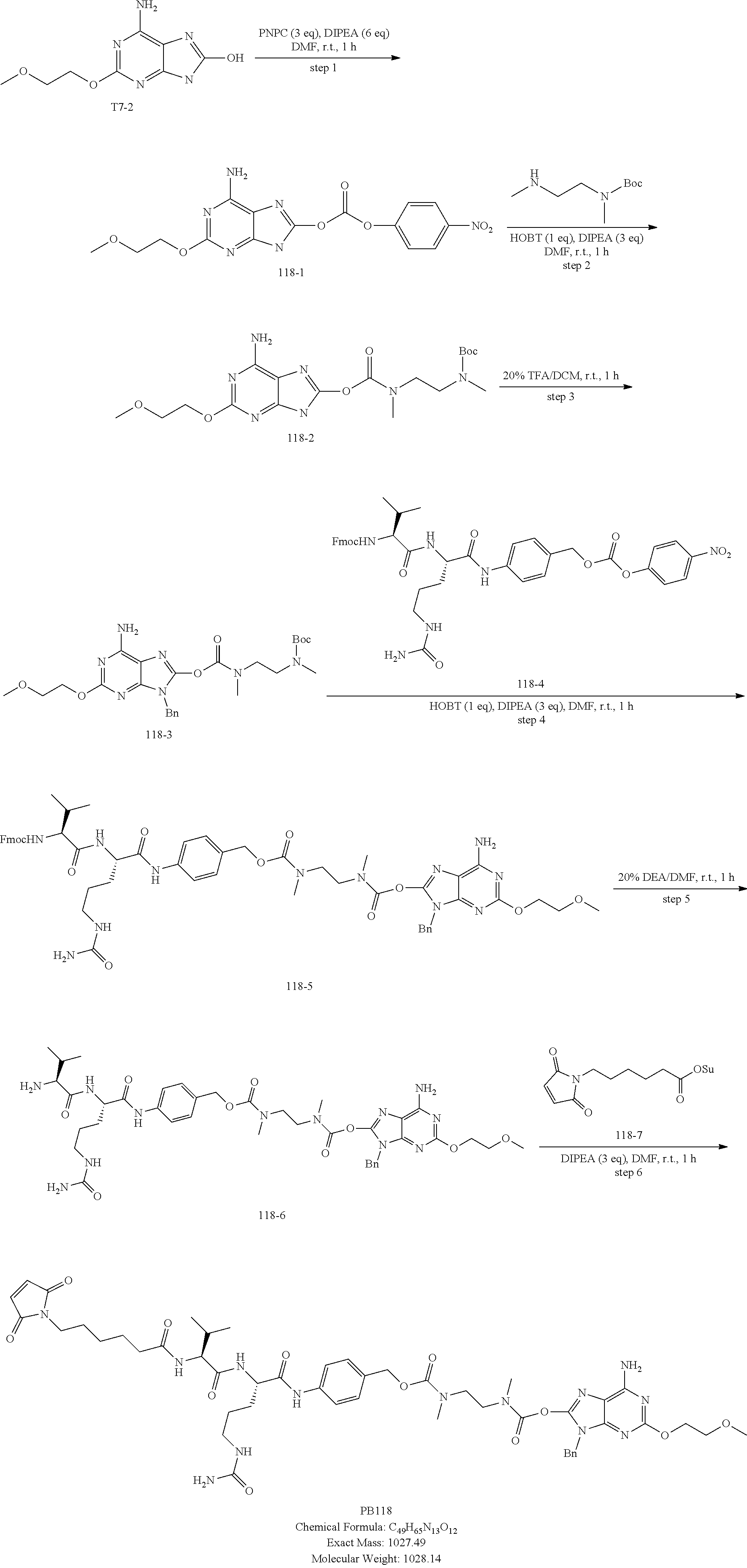

A Drug-Linker attached to T7-2 (PB118) was prepared as follows:

### Step 1

A solution of T7-2 (300 mg, 0.951 mmol) and DIPEA (737.8 mg, 5.706 mmol) in anhydrous DMF (6 mL) was stirred at room temperature for 5 min, then PNPC (868.26 mg, 2.853 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (50 ml) at 5° C. for 30 minutes. The solution was filtered and the filter cake was washed with acetonitrile to yield compound 118-1 (270 mg, 0.562 mmol, 59.08%) as a white solid. purity =90%-93%.

### Step 2

A solution of compound 118-1 (270 mg, 0.562 mmol), N-Boc-N,N′-dimethylethylenediamine (105.80 mg, 0.562 mmol) and HOBT (75.94 mg, 0.562 mmol) in anhydrous DMF (5 mL) was stirred at room temperature, then DIPEA (217.90 mg, 1.686 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 118-2 (210 mg, 0.397 mmol, 70.71%) as a white solid. purity=90%-95%.

### Step 3

A solution of compound 118-2 (210 mg, 0.397 mmol) and TFA (1 mL) in anhydrous DCM (4 mL) was stirred at room temperature for 1 hr until LCMS indicated all starting amine was consumed and the desired product was detected. Then the solution was concentrated to dryness to yield compound 118-3 (170 mg, 0.397 mmol, 100%) as a yellow oil, which was used as such in the next step. purity=85%-90%.

### Step 4

A solution of compound 118-3 (170 mg, 0.396 mmol), compound 118-4 (303.65 mg, 0.396 mmol) and HOBT (53.51 mg, 0.396 mmol) in anhydrous DMF (5 mL) was stirred at room temperature, then DIPEA (153.54 mg, 1.188 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 118-5 (95 mg, 0.090 mmol, 22.73%) as a white solid. purity=80%-90%.

### Step 5

A solution of compound 118-5 (95 mg, 0.090 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 118-6 (75 mg, 0.090 mmol, 100%) as a colorless oil, which was used as such in the next step. purity=80%-90%.

### Step 6

A solution of compound 118-6 (75 mg, 0.090 mmol) and DIPEA (34.83 mg, 0.270 mmol) in anhydrous DMF (1 mL) was stirred at room temperature for 5 min, then a solution of compound 118-7 (41.54 mg, 0.135 mmol) in anhydrous DMF (1 mL) was added dropwise by syringe over 2 min. The resulting solution was stirred for another 1 hr at r.t. until LCMS indicated all starting amine was consumed and the mass of desired product was detected. The resulting solution was neutralized with formic acid to adjust the pH to 6-7. Then the reaction solution was purified by Prep-HPLC to yield compound PB118 (10 mg, 0.001 mmol, 10.87%) as a white solid. LCMS, m/z=1028.55 (M+H)⁺.

### Example 50: Preparation of a Drug-Linker Containing a PEG Unit and a Cleavable Linker Attached to T7-2 (PB119)

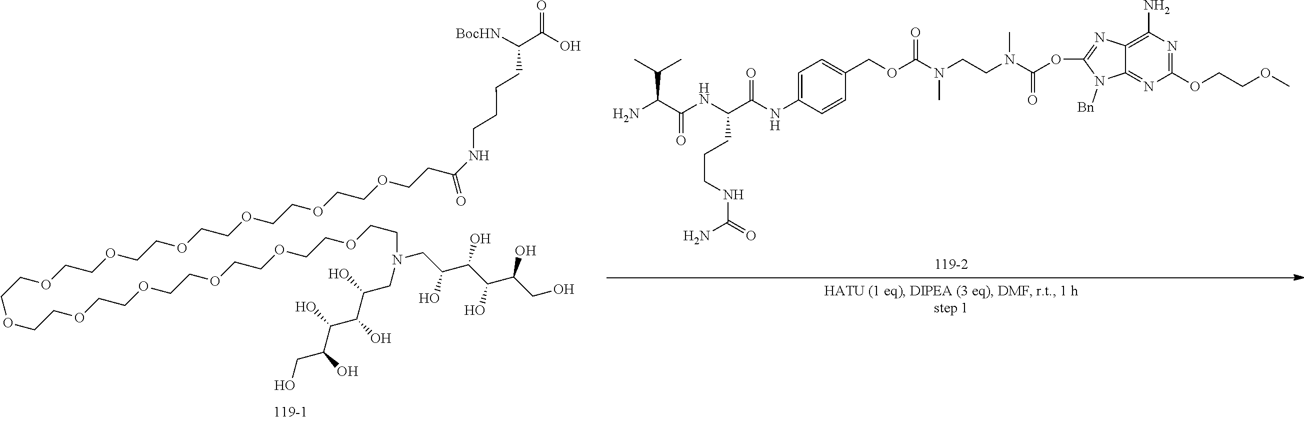 [SMILES: CC(C)(C)OC(=O)N[C@@H](CCCCNC(=O)CCOCCOCCOCCOCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)O ; COCCOc1nc(N)c2nc(OC(=O)N(C)CCN(C)C(=O)OCc3ccc(NC(=O)[C@H](CCCNC(N)=O)NC(=O)[C@@H](N)C(C)C)cc3)n(Cc3ccccc3)c2n1]

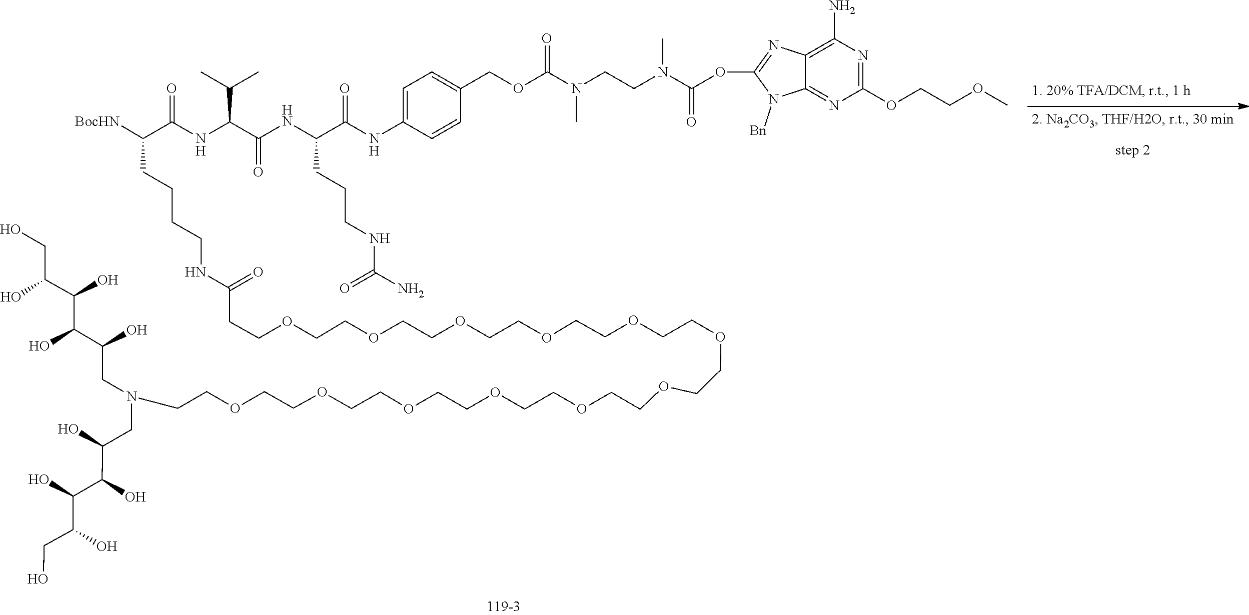 [SMILES: COCCOc1nc(N)c2nc(OC(=O)N(C)CCN(C)C(=O)OCc3ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)OC(C)(C)C)C(C)C)cc3)n(Cc3ccccc3)c2n1]

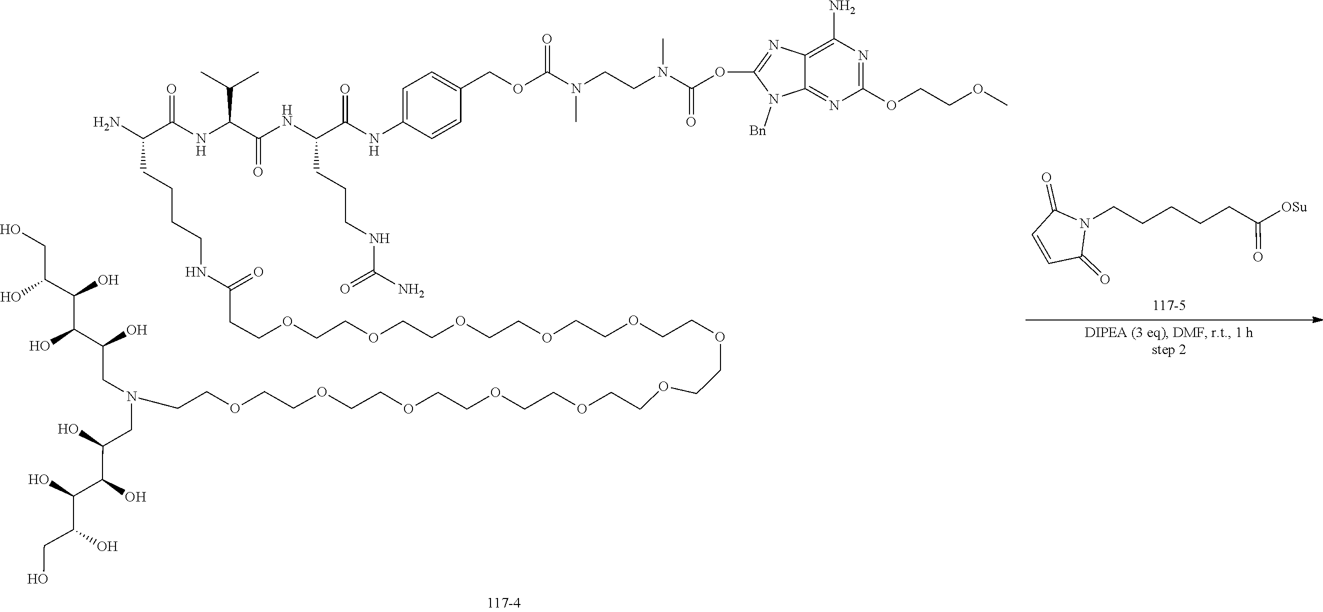

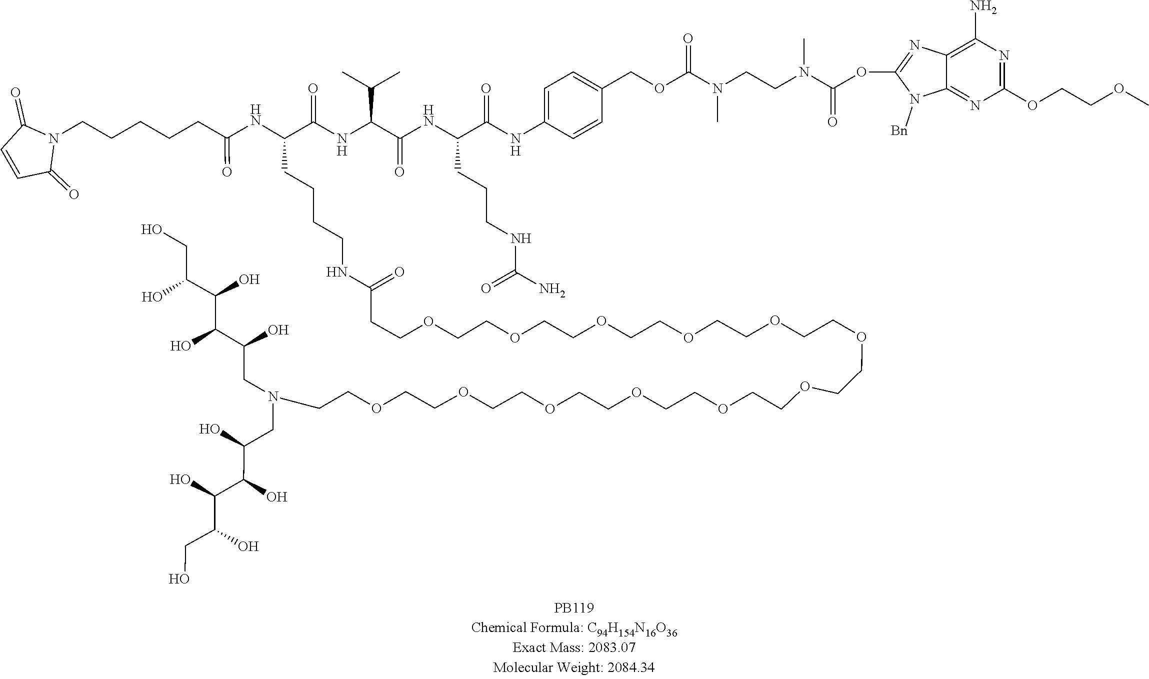 [SMILES: COCCOc1nc(N)c2nc(OC(=O)N(C)CCN(C)C(=O)OCc3ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN4C(=O)C=CC4=O)C(C)C)cc3)n(Cc3ccccc3)c2n1]

A Drug-Linker containing a PEG unit and a cleavable linker attached to T7-2 (PB119) was prepared as follows:

### Step 1

A solution of compound 119-1 (154.7 mg, 0.132 mmol), compound 119-2 (110 mg, 0.132 mmol) and HATU (50.1 mg, 0.132 mmol) in anhydrous DMF (2 mL) was stirred at room temperature for 5 min, then DIPEA (51.1 mg, 0.395 mmol) was added. The resulting solution was stirred for another 1 hr at r.t. until LCMS indicated complete reaction. The reaction solution was purified directly by reverse phase liquid chromatography to yield compound 119-3 (75 mg, 0.038 mmol, 28.63%) as a white solid. purity=90%-95%.

### Step 2

A solution of compound 119-3 (75 mg, 0.038 mmol) and TFA (1 mL) in anhydrous DCM (4 mL) was stirred at room temperature for 1 hr until LCMS indicated all starting amine was consumed and the desired product (m/z=631=1891/3+H) along with sugar-esterificated product (TFA was condensed with hydroxy group in sugar unit, mono-ester with m/z=(1891+96)/3+H=663) were formed. The completed reaction solution was condensed to dryness and then redissolved in THF (4 mL) and water (2 mL), treated with saturated aqueous sodium carbonate solution to adjust the pH to 8-9. The resulting solution was stirred at room temperature for 30 min to achieve complete hydrolysis. The solution was then neutralized with diluted TFA and condensed, the residue was purified by reverse phase liquid chromatography to yield compound 119-4 (41 mg, 0.022 mmol, 57.75%) as a white solid. purity=80%-85%.

### Step 3

A solution of compound 119-4 (41 mg, 0.022 mmol) and DIPEA (8.5 mg, 0.066 mmol) in anhydrous DMF (2 mL) was stirred at room temperature for 5 min, then a solution of compound 119-5 (10.0 mg, 0.033 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield compound PB119 (9 mg, 0.004 mmol, 19.92%) as a white solid. LCMS, m/z=1043.05 (M/2+H)⁺.

### Example 51: Preparation of a Drug-Linker Containing a PEG Unit and a Cleavable Linker Attached to T7-1 (PB120)

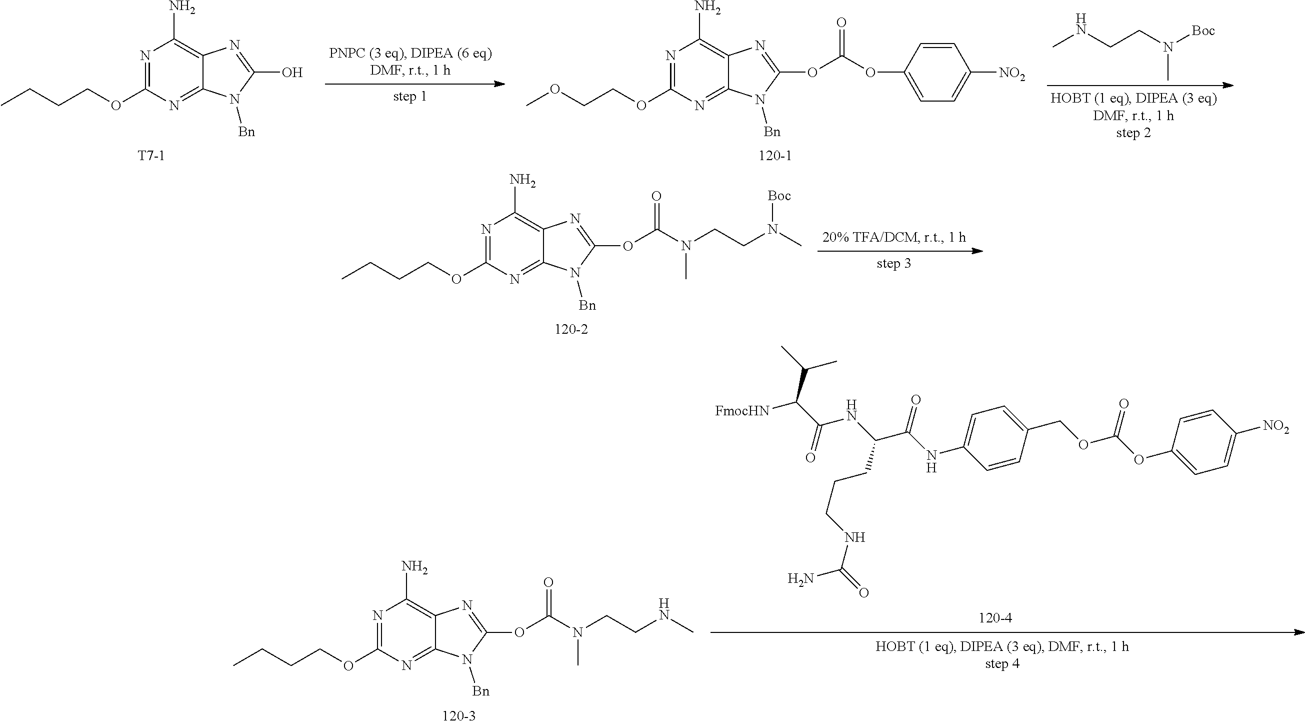

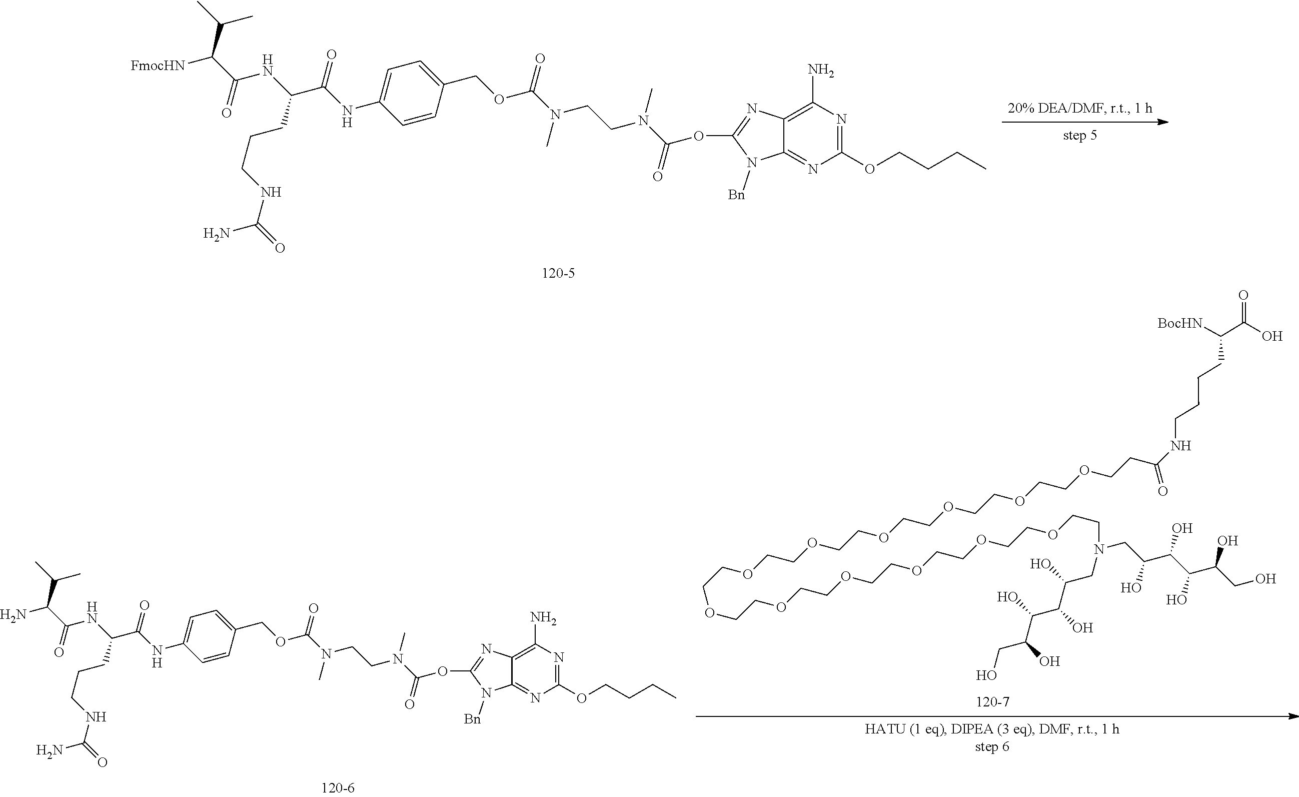

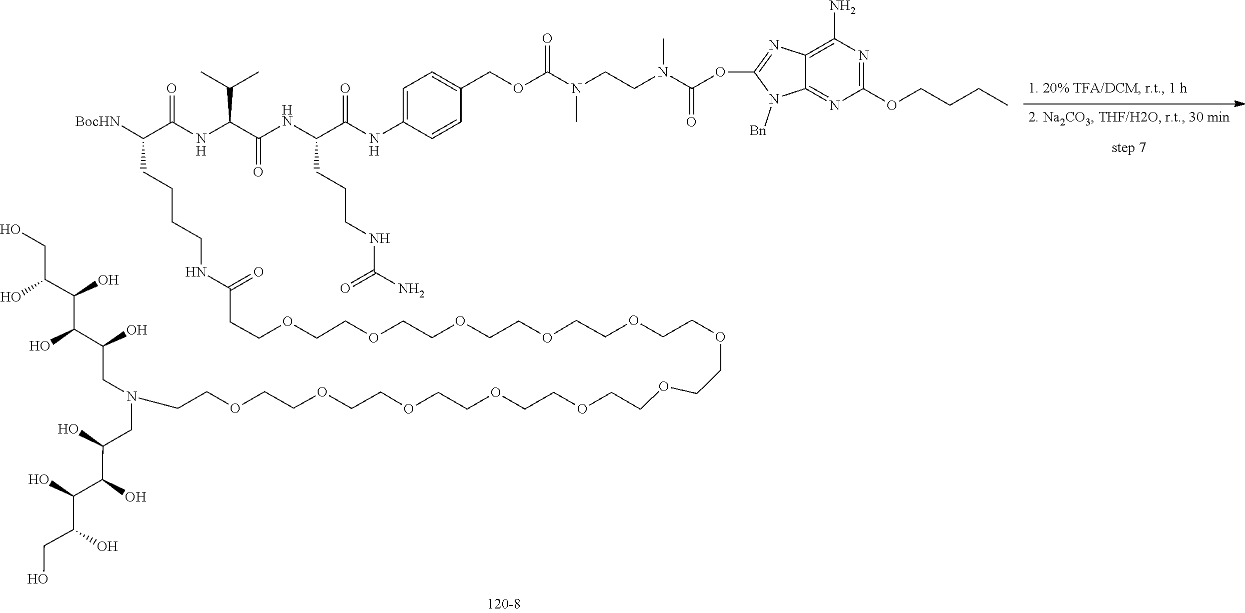 [SMILES: CCCCOc1nc(N)c2nc(OC(=O)N(C)CCN(C)C(=O)OCc3ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)OC(C)(C)C)C(C)C)cc3)n(Cc3ccccc3)c2n1]

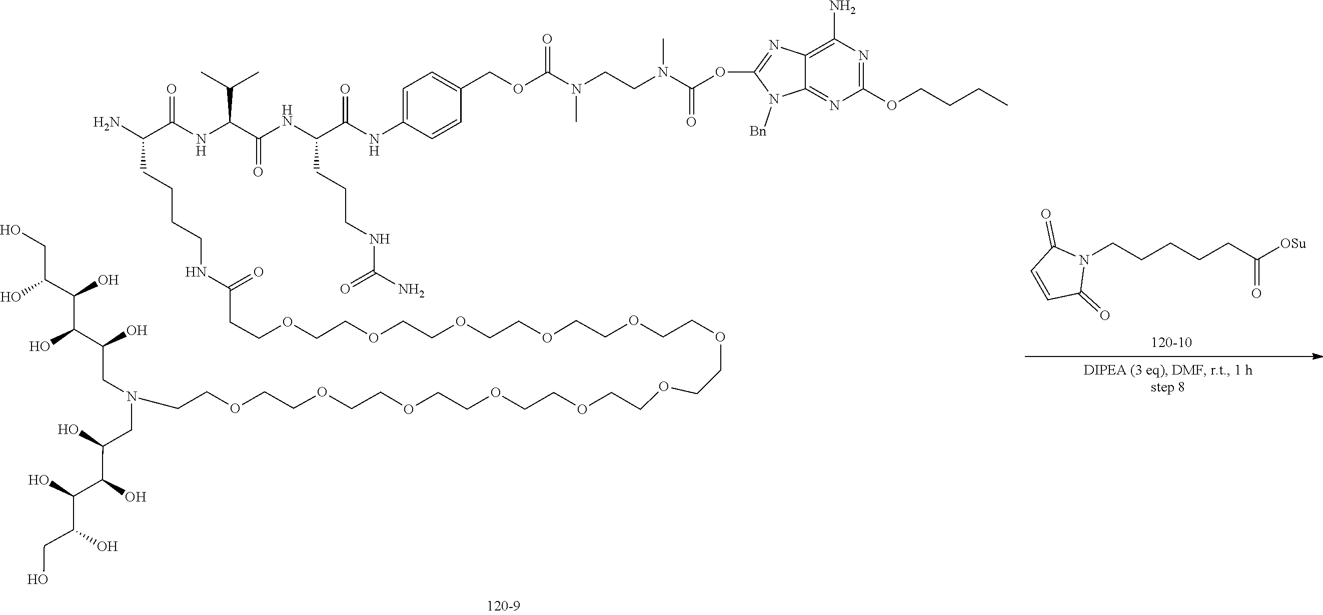

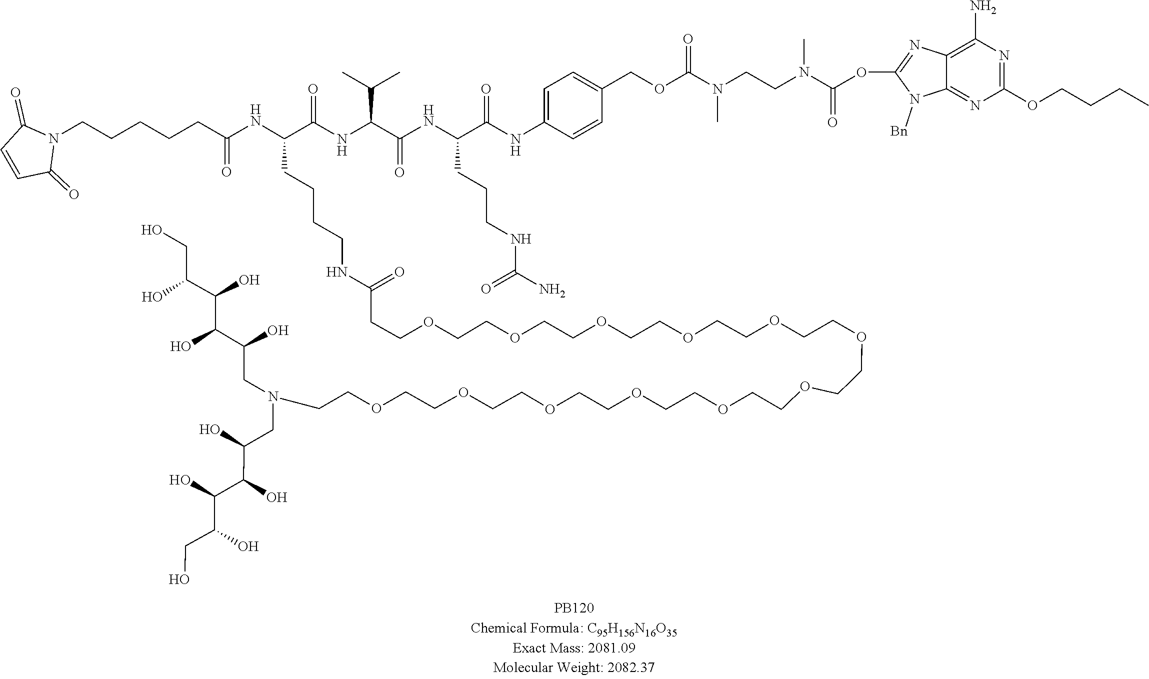 [SMILES: CCCCOc1nc(N)c2nc(OC(=O)N(C)CCN(C)C(=O)OCc3ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN4C(=O)C=CC4=O)C(C)C)cc3)n(Cc3ccccc3)c2n1]

A Drug-Linker containing a PEG unit and a cleavable linker attached to T7-1 (PB120) was prepared as follows:

### Step 1

A solution of T7-1 (3 g, 9.574 mmol) and DIPEA (7.4 g, 57.444 mmol) in anhydrous DMF (30 mL) was stirred at room temperature for 5 min, then PNPC (8.7 g, 28.722 mmol) was added. The resulting solution was stirred for another 1 hr at r.t. until LCMS indicated all starting amine was consumed and the desired product was detected. Then most of the DMF and DIPEA was evaporated and the residue was washed in acetonitrile (50 ml) at 5° C. for 30 minutes. The solution was filtered and the filter cake was washed with acetonitrile to yield compound 120-1 (4.1 g, 8.569 mmol, 89.52%) as a white solid. purity=90%-95%.

### Step 2

A solution of compound 120-1 (4.1 g, 8.569 mmol), N-Boc-N,N′-dimethylethylenediamine (1.61 g, 8.569 mmol) and HOBT (1.16 g, 8.569 mmol) in anhydrous DMF (10 mL) was stirred at room temperature, then DIPEA (3.3 g, 25.707 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 120-2 (2.3 g, 4.359 mmol, 50.88%) as a white solid. purity=90%-95%.

### Step 3

A solution of compound 120-2 (2.3 g, 4.359 mmol) and TFA (4 mL) in anhydrous DCM (16 mL) was stirred at room temperature for 1 hr until LCMS indicated all starting amine was consumed and the desired product was detected. Then the solution was concentrated to dryness to yield compound 120-3 (1.8 g, 4.210 mmol, 96.77%) as a yellow oil, which was used as such in the next step. purity=85%-90%.

### Step 4

A solution of compound 120-3 (1.8 g, 4.210 mmol), compound 120-4 (3.2 g, 4.210 mmol) and HOBT (0.57 g, 4.210 mmol) in anhydrous DMF (15 mL) was stirred at room temperature, then DIPEA (1.6 g, 12.630 mmol) was added. The resulting solution was stirred for another 1 hr at r.t. until LCMS indicated all starting amine was consumed and the desired product was detected. The reaction solution was terminated and purified directly by reverse phase liquid chromatography to yield compound 120-5 (270 mg, 0.256 mmol, 6.14%) as a white solid. purity=85%-90%.

### Step 5

A solution of compound 120-5 (270 mg, 0.256 mmol) and DEA (1 mL) in anhydrous DMF (4 mL) was stirred at room temperature for 1 hr until LCMS indicated all starting amine was consumed and the desired product was detected. Then the solution was concentrated to dryness to yield compound 120-6 (210 mg, 0.252 mmol, 98.59%) as a colorless oil, which was used as such in the next step. purity=85%-90%.

### Step 6

A solution of compound 120-7 (296 mg, 0.252 mmol), compound 120-6 (210 mg, 0.252 mmol) and HATU (95.9 mg, 0.252 mmol) in anhydrous DMF (5 mL) was stirred at room temperature for 5 min, then DIPEA (97.8 mg, 0.756 mmol) was added. The resulting solution was stirred for another 1 hr at r.t. until LCMS indicated complete reaction. The reaction solution was purified directly by reverse phase liquid chromatography to yield compound 120-8 (120 mg, 0.060 mmol, 23.95%) as a white solid. purity=90%-95%.

### Step 7

A solution of compound 120-8 (120 mg, 0.060 mmol) and TFA (1 mL) in anhydrous DCM (4 mL) was stirred at room temperature for 1 hr until LCMS indicated all starting amine was consumed and the desired product (m/z=630=1889/3+H) along with sugar-esterificated product (TFA was condensed with hydroxy group in sugar unit, mono-ester with m/z=(1889+96)/3+H=662) were formed. The completed reaction solution was condensed to dryness and then redissolved in THF (4 mL) and water (2 mL), treated with saturated aqueous sodium carbonate solution to adjust the pH to 8-9. The resulting solution was stirred at room temperature for 30 min to achieve complete hydrolysis. The solution was then neutralized with diluted TFA and condensed, the residue was purified by reverse phase liquid chromatography to yield compound 120-9 (45 mg, 0.024 mmol, 39.47%) as a white solid. purity=90%-95%.

### Step 8

A solution of compound 120-9 (45 mg, 0.024 mmol) and DIPEA (9.2 mg, 0.072 mmol) in anhydrous DMF (2 mL) was stirred at room temperature for 5 min, then a solution of compound 120-10 (11.0 mg, 0.036 mmol) in anhydrous DMF (1 mL) was added dropwise by syringe over 5 min. The resulting solution was stirred for another 1 hr at r.t. until LCMS indicated all starting amine was consumed and the mass of desired product was detected. Then the reaction solution was purified by Prep-HPLC to yield PB120 (15 mg, 0.007 mmol, 30.24%) as a white solid. LCMS, m/z=1042.19 (M/2+H)⁺.

### Example 52: Preparation of a Conjugate (PA003) of Drug-Linker PB003 and an Antibody

A solution of 10 mg/mL of antibody in pH 7.1 PB 5 mM EDTA buffer was reduced by 10 mM TCEP at 25° C. for 120 minutes. 6.5eq of 5 mM PB03 in DMA was added to the reduced antibody solution, and the resulting mixture was stirred at 25° C. for 120 min. The ADC was purified with PD-10 column. The ADC is named PA003.

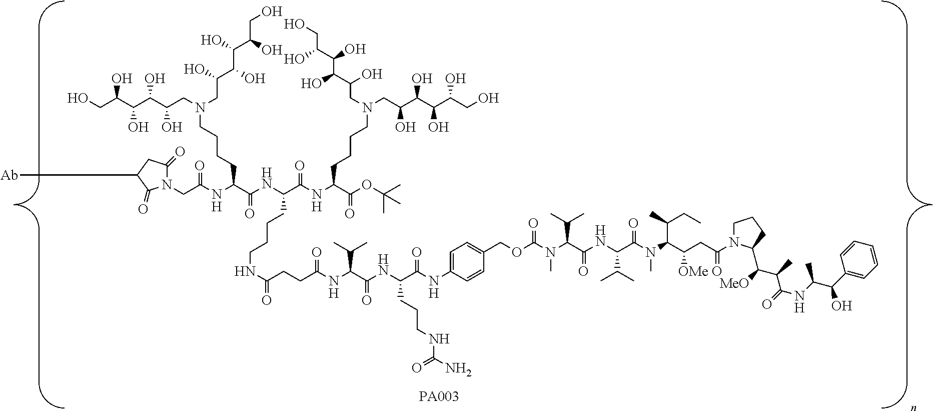

### Example 53: Preparation of a Conjugate (PA004) of Drug-Linker PB004 and an Antibody

A solution of 10 mg/mL of antibody in pH 7.1 PB 5 mM EDTA buffer was reduced by 10 mM TCEP at 25° C. for 120 minutes. 6.5eq of 5 mM PB004 in DMA was added to the reduced antibody solution, and the resulting mixture was stirred at 25° C. for 120 min. The ADC was purified with PD-10 column. The ADC is named PA004.

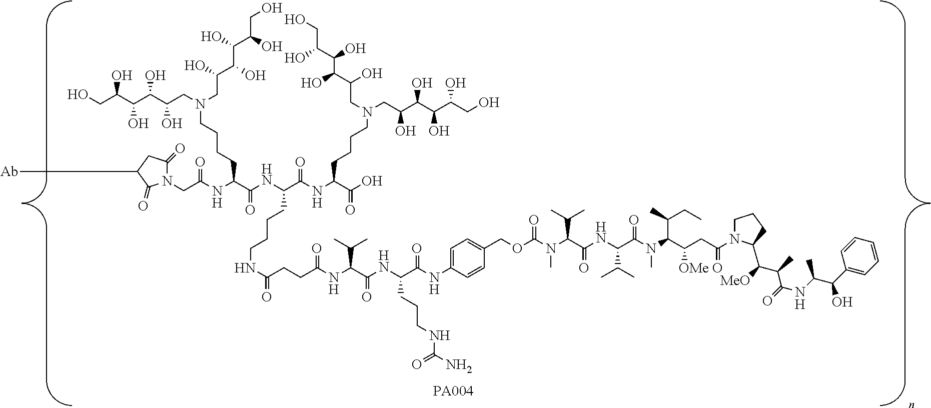

### Example 54: Preparation of a Conjugate (PA008) of Drug-Linker PB008 and an Antibody

A solution of 10 mg/mL antibody in pH 7.1 PB 5 mM EDTA buffer was reduced by 10 mM TCEP at 25° C. for 120 minutes. 6.5eq of 5 mM PB08 in DMA was added to the reduced antibody solution, and the resulting mixture was stirred at 25° C. for 120 min. The ADC was purified with PD-10 column. The ADC is named PA008.

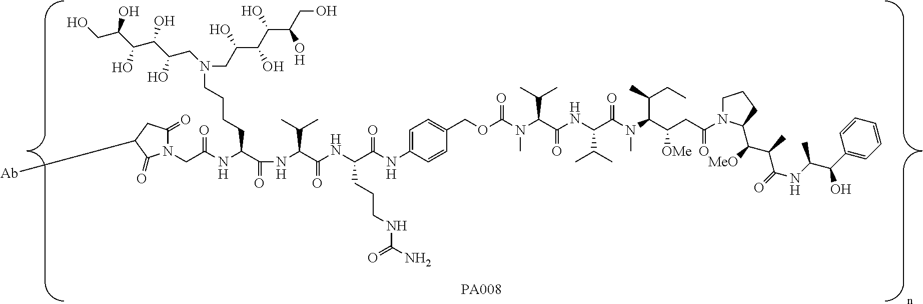

### Example 55: Preparation of a Conjugate (PA038) of Drug-Linker PB0038 and an Antibody

A solution of 10 mg/mL antibody in pH 7.1 PB 5 mM EDTA buffer was reduced by 10 mM TCEP at 25° C. for 120 minutes. TCEP was removed with PD-10 column. 15eq of 5 mM PB038 in DMA was added to the reduced antibody solution, and the resulting mixture was stirred at 25° C. for 120 min. The ADC was purified with PD-10 column. The ADC is named PA038.

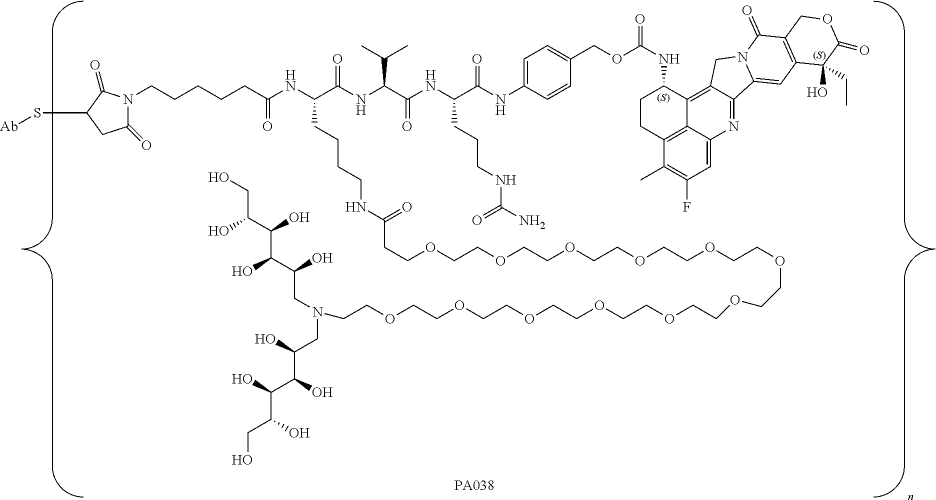

ADC PA0038 was analyzed by HIC-HPLC and SEC-HPLC.

### Example 56: Preparation of a Conjugate (PA039) of Drug-Linker PB039 and an Antibody

A solution of 10 mg/mL antibody in pH 7.1 PB 5 mM EDTA buffer was reduced by 10 mM TCEP at 25° C. for 120 minutes. TCEP was removed with PD-10 column. 15eq of 5 mM PB039 in DMA was added to the reduced antibody solution and the resulting mixture was stirred at 25° C. for 120 min. The ADC was purified with PD-10 column. The ADC is named PA039.

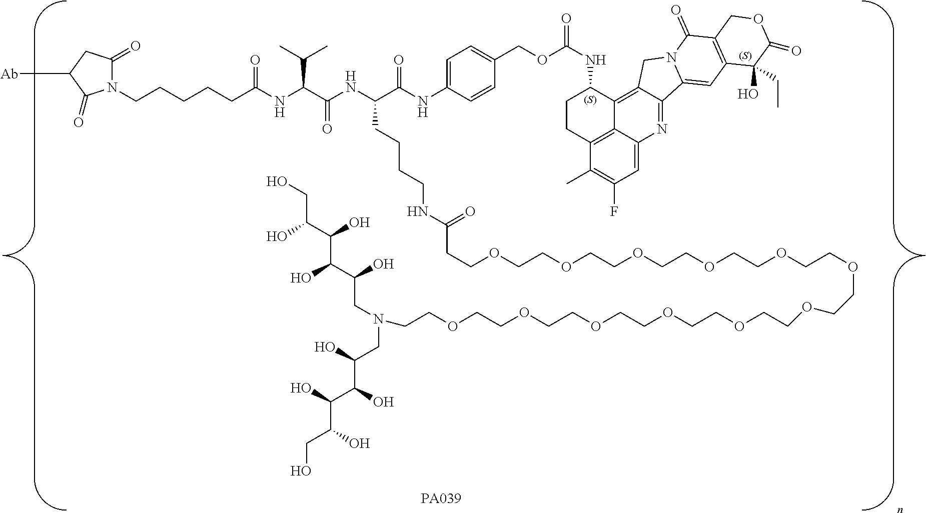

ADC PA0039 was analyzed by HIC-HPLC and SEC-HPLC.

### Example 57: Preparation of a Conjugate (PA040) of Drug-Linker PB040 and an Antibody

A solution of 10 mg/mL antibody in pH 7.1 PB 5 mM EDTA buffer was reduced by 10 mM TCEP at 25° C. for 120 minutes. TCEP was removed with a PD-10 column. 15eq of 5 mM PB040 in DMA was added to the reduced antibody solution, and the resulting mixture was stirred at 25° C. for 120 min. The ADC was purified with PD-10 column. The ADC is named PA040.

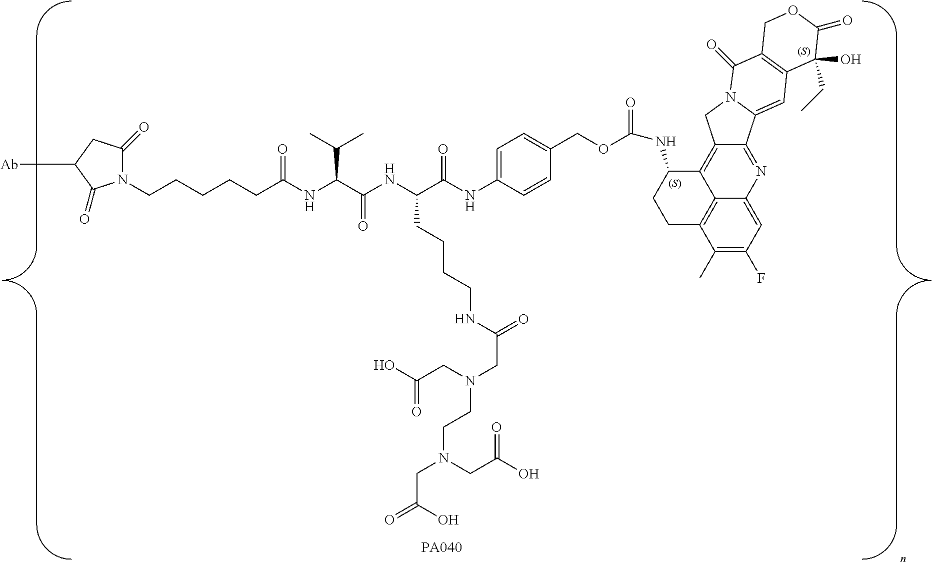

### Example 58: Preparation of a Conjugate (PA082) of Drug-Linker PB082 and an Antibody

A solution of 10 mg/mL antibody in pH7.1 PB 5 mM EDTA Buffer was reduced by 10 mM TCEP at 25° C. for 120 minutes. TCEP was removed with a PD-10 column. 15eq of 5 mM PB082 in DMA was added to the reduced antibody solution, and the resulting mixture was stirred at 25° C. for 120 min. The ADC was purified with PD-10 column. The ADC is named PA082.

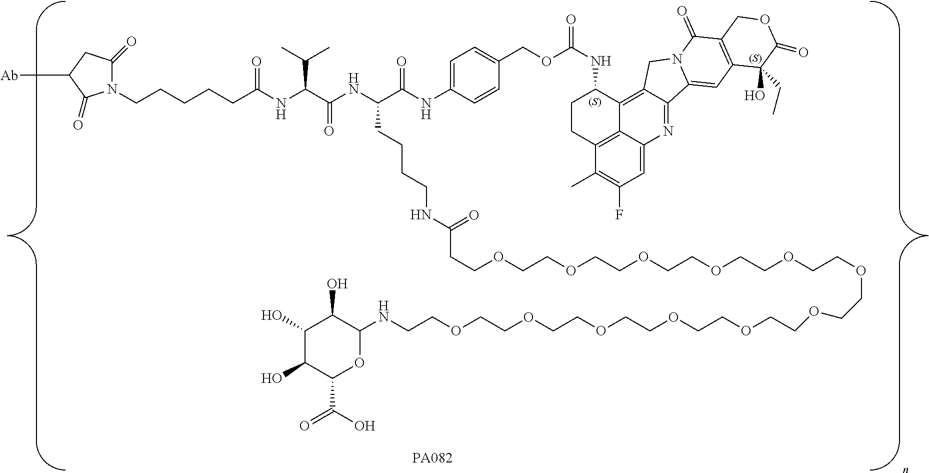

### Example 59: Preparation of a Conjugate (PA082) of Drug-Linker PB083 and an Antibody

A solution of 10 mg/mL antibody in pH 7.1 PB 5 mM EDTA buffer was reduced by 10 mM TCEP at 25° C. for 120 minutes. TCEP was removed with a PD-10 column. 15eq of 5 mM PB083 in DMA was added to the reduced antibody solution, and the resulting mixture was stirred at 25° C. for 120 min. The ADC was purified with a PD-10 column. The ADC is named PA083.

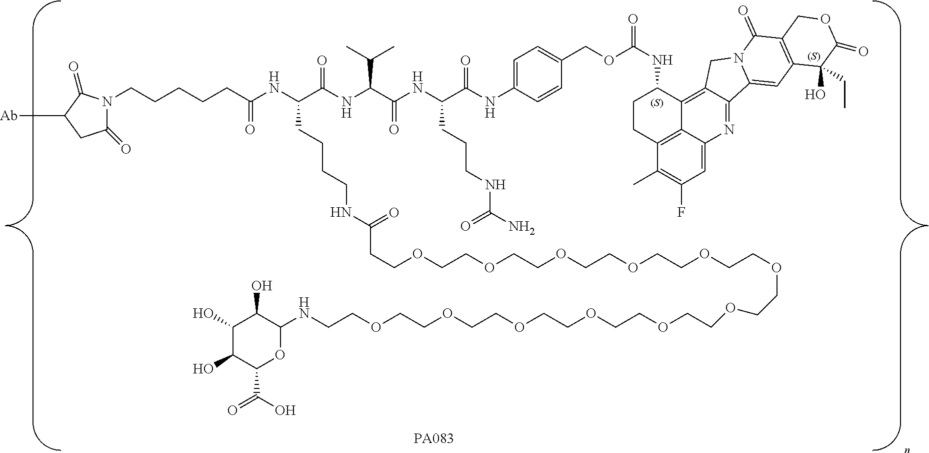

### Example 60: Stability Testing of ADC PA038

When stored at 37° C. for up to 15 days or undergoing 5 cycles of freeze-thaw, ADC PA038 appeared to be stable by both HIC and SEC. No significant increase in aggregation was observed over time at 37° C. or multiple freeze-thaw cycles. At 100 mg/mL, PA038 was found to be stable with no aggregation (by SEC) or precipitation

### Example 61: Retention Times Measured by HIC-HPLC

At DAR8 the relative retention time of ADCs PA038 and PA039 was shorter than that of the DAR2 species of the corresponding MC-VC-PAB-MMAE based ADC.

HIC-HPLC conditions: Column: TSKgel Butyl-NPR, 2.5 μm, 4.6 mm×100 mm (PN: 42168); Column temperature: 30° C.; UV: 280 nm; Mobile phase: A: 50 mM PB, 1.5M (NH₄)₂SO₄,pH=7.0; B: 50 mM PB(pH=7.0):IPA=75:25(v:v) Flow rate:0.5 mL/min; Gradient elution: 0 min→20 min(0% B→100% B), 20 min→21 min(100% B→0% B), 21 min→32 min(0% B).

### Example 62: In Vitro Cytotoxicity Assays of an Anti-FOLR1 Immunoconjugate

The ability of an anti-FOLR1 conjugate to inhibit cell growth was measured using in vitro cytotoxicity assays. The assay method was as follows: OV90, OVCAR-3 and NCI-H292 cells were harvested and seeded into solid white, flat bottom 96-well plates at the indicated amounts (according to cell growth rates) prior to adding an anti-FOLR1 conjugate. The next day, the cells were exposed to the conjugate at a drug concentration range of 30 μg/ml to 0.37 μg/ml or 100 μg/ml to 0.015 μg/ml, with 1:3 serial dilutions and duplicate wells. The plates were incubated at 37° C. for 120h. After incubation, 40 i9 L of CTG (Promega, G7572) per well was added to the plates and the plates were read on an MD SpectraMax 13× after a 5 min incubation. Growth inhibition was calculated as percentages of growth relative to untreated cells using Microsoft Excel and Prism software.

The anti-FOLR1 conjugate was prepared by conjugating drug-linker PA038 to antibody F131 to make conjugate F131-LD038. 2 mL of antibody (F131, 10 mg/mL) in 50 mM sodium phosphate buffer containing 5 mM EDTA (pH=6.9) was added to the aqueous solution of 10 mM TCEP HCl (Tris(2-carboxyethyl) phosphine HCl), at the molar ratio of TCEP:mAb=8.0. The reducing reaction proceeded for 2 hours at 25° C. The excess TCEP and its byproduct was removed by ultrafiltration with 50 mM sodium phosphate buffer (pH=6.9). LD038 (salt of TFA) was dissolved in water at a concentration of 20 mg/mL and added to the reduced mAb (F131) at a molar ratio of 7.7 (LD038: F131). The coupling reaction was stirred for 2 hours at 25° C. The excess LD038 and its impurities were removed by ultrafiltration with 50 mM sodium phosphate buffer. The ADC was stored in 20 mM histidine buffer containing 6% sucrose and 0.02% (w/V) Tween 20 by UFDF. The purity of the ADC as determined by SEC-HPLC was 97.5% and DAR value as determined by LC-MS was 7.6. The drug loading was approximately 8.

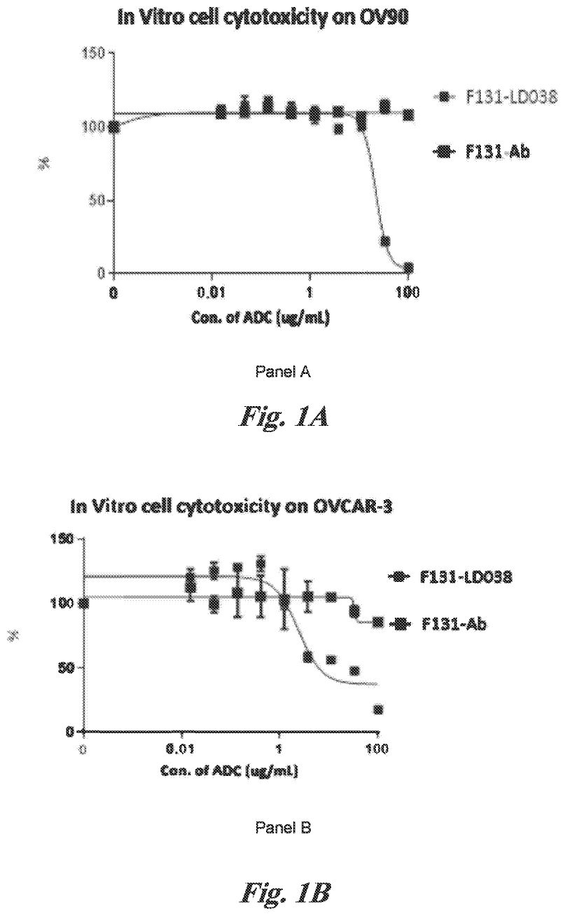

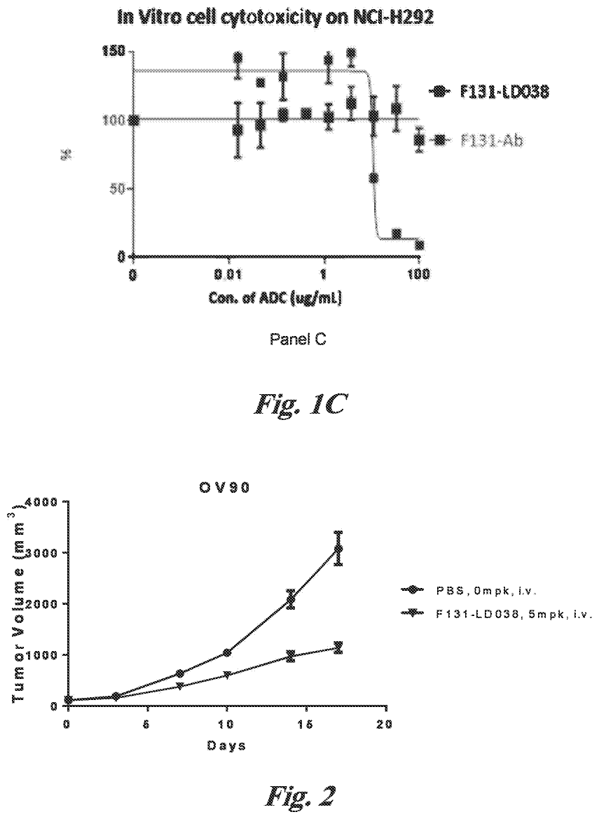

The results are shown in FIGS. 1A, 1B and 1C. The anti-FOLR1 conjugate had a significantly better cytotoxicity on OV90, OVCAR-3 and NCI-H292 cells compared with the naked (unconjugated) antibody (F131 Ab).

### Example 63. In Vivo Efficacy of an Anti-FOLR1 Conjugate in an OV90 Xenograft Model

The in vivo anti-tumor efficacy of an anti-FOLR1 conjugate was evaluated in the subcutaneous OV90 human ovarian cancer xenograft model in female BALB/c nude mice. OV90 tumor cells (ATCC, Manassas, VA, cat #CRL-11732) were maintained in vitro as a monolayer culture in RPMI 1640 medium supplemented with 10% heat inactivated fetal bovine serum, at 37° C. in an atmosphere of 5% CO₂ in air. The tumor cells were routinely sub-cultured twice weekly by trypsin-EDTA treatment. The cells growing in an exponential growth phase were harvested and counted for tumor cell inoculation.

The anti-FOLR1 conjugate was prepared by conjugating drug-linker PA038 to antibody F131 to make conjugate F131-LD038 (as described above). The drug loading was approximately 8.

Each mouse was inoculated subcutaneously at the right flank with OV90 tumor cells (2×10⁶) in 0.1 mL of 1640 supplemented with Matrigel (1:1) for tumor development. Treatments were started on day 8 after tumor cell inoculation when the average tumor size reached approximately 117 mm³. The animals were assigned to one of 2 groups using an Excel-based randomization software program performing stratified randomization based upon their tumor volumes. Each group consisted of 7 tumor-bearing mice.

The anti-FOLR1 immunoconjugate was administered to the mice in 4 doses at 5 mg/kg; dosing was carried out at day 0, 3, 7 and 10 after grouping.

During routine monitoring, the animals were checked three times per week for any effects on tumor growth and on normal behavior such as mobility, food and water consumption (by observation only), body weight gain/loss (body weights were measured every other day), eye/hair matting and any other abnormal effects as stated in the protocol. Death and observed clinical signs were recorded on the basis of the numbers of animals within each subset. Tumor size was measured twice weekly in two dimensions using a caliper, and the volume was expressed in mm³ using the formula: V=0.5a×b², where a and b are the long and short diameters of the tumor, respectively.

The median tumor volume of the different treatment groups is plotted in FIG. 2. Treatment with conjugate F131-LD038 resulted in a significant decrease in median tumor volume as compared to the PBS control group.

### Example 64. In Vivo Efficacy of an Anti-FOLR1 Conjugate in an NCI-H292 Xenograft Model

The in vivo anti-tumor efficacy of an anti-FOLR1 conjugate was evaluated in the subcutaneous NCI-H292 human lung cancer xenograft model in female BALB/c nude mice. NCI-H292 tumor cells (ATCC, Manassas, VA, cat #CRL-1848) were maintained in vitro as a monolayer culture in RPM11640 medium supplemented with 10% heat inactivated fetal bovine serum, at 37° C. in an atmosphere of 5% CO₂ in air. The tumor cells were routinely sub-cultured twice weekly by trypsin-EDTA treatment. The cells growing in an exponential growth phase were harvested and counted for tumor cell inoculation.

The anti-FOLR1 conjugate was prepared by conjugating drug-linker PA038 to antibody F131 to make conjugate F131-LD038 (as described above). The drug loading was approximately 8.

Each mouse was inoculated subcutaneously at the right flank with NCI-H292 tumor cells (10×10⁶) in 0.1 mL of RPM 11640 for tumor development. Treatment was started on day 11 after tumor cell inoculation when the average tumor size reached approximately 123 mm³. The animals were assigned to one of 2 groups using an Excel-based randomization software program performing stratified randomization based upon their tumor volumes. Each group consisted of 6 tumor-bearing mice.

The anti-FOLR1 immunoconjugate was administered to the mice with 4 doses at 5 mg/kg; dosing was carried out at day 0, 3, 7 and 10 after grouping.

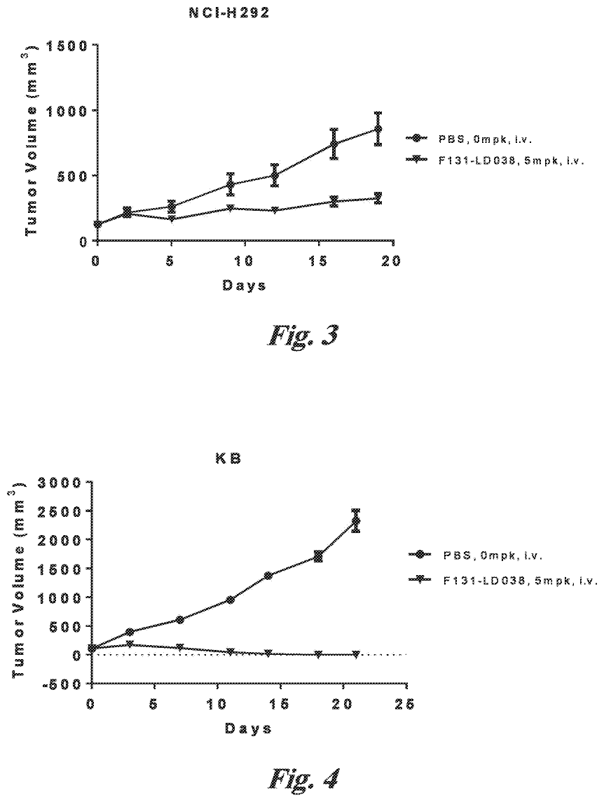

The median tumor volumes of the different treatment groups are plotted in FIG. 3. Treatment with the F131-LD038 conjugate resulted in a significant decrease in median tumor volume as compared to the PBS control group.

### Example 65. In Vivo Efficacy of an Anti-FOLR1 Conjugate in an NCI-H292 Xenograft Model

The in vivo anti-tumor efficacy of an anti-FOLR1 conjugate was evaluated in a subcutaneous KB (human oral epidermoid carcinoma cell) xenograft model in female BALB/c nude mice. KB tumor cells (ATCC, CCL-17) were maintained in vitro as a monolayer culture in MEM medium supplemented with 10% heat inactivated fetal bovine serum, at 37° C. in an atmosphere of 5% CO₂ in air. The tumor cells were routinely sub-cultured twice weekly by trypsin-EDTA treatment. The cells growing in an exponential growth phase were harvested and counted for tumor cell inoculation.

The anti-FOLR1 conjugate was prepared by conjugating drug-linker PA038 to antibody F131 to make conjugate F131-LD038 (as described above). The drug loading was approximately 8.

Each mouse was inoculated subcutaneously at the right flank with KB tumor cells (1×106) in 0.1 mL of RPM11640 for tumor development. Treatments were started on day 13 after tumor cell inoculation when the average tumor size reached approximately 110 mm³. The animals were assigned to one of 2 groups using an Excel-based randomization software program performing stratified randomization based upon their tumor volumes. Each group consisted of 6 tumor-bearing mice.

The anti-FOLR1 immunoconjugate was administer to the mice with 4 doses at 5 mg/kg; dosing was carried out at days 0, 3, 7 and 10 after grouping.

The median tumor volume of the different treatment groups is plotted in FIG. 4. Treatment with the F131-LD038 conjugate resulted in a significant decrease in median tumor volume as compared to the PBS control group. All the 6 mice had a complete response after 20 days of treatment.

### Example 66. Pharmacokinetics (PK) of an Anti-FOLR-1 Conjugate in Rat

Rats were purchased from VR and used 1 week after housing. The rats were group-housed in sterilized cages and maintained under specific pathogen-free conditions. In the experimental room, the environmental conditions were as follows: a temperature of 20° C.-22° C. and humidity of 59%-78% humidity, with artificial illumination for 12 h. The rat cage was a polysulfone box, which was used after autoclaving, with the specification of 325 mm×210 mm×180 mm, with up to 3 animals raised in each box. The experiment number, experimental start time, project leader, experimental personnel, animal source, group and animal number were indicated on the cage card. The experimental animals were ear-marked. The rats were fed an FR-2 diet and were provided tap-water (used after autoclaving). Their body weights were approximately 290 g at dosing.

2 groups with 3 rats per group were treated with single dose of F131-LD038 immunoconjugate (prepared as described above) or naked antibody F131 at 3 mg/kg intravenously (IV). Blood samples were collected at 10 min, 4h, 1d, 4d, 7d, 10d, 14d, and 21 days after administration, followed by centrifugation (4° C., 10000×g, 3 min) to separate the plasma. Total antibody concentration of each conjugate or mAb in serum was detected by ELISA and analyzed by GraphPad Prism software.

Goat anti-human IgG Fc (Invitrogen, 31125) was coated on a 96-well micro plate (Thermo, cat:468667) in PBS with 2 μg/ml, 100 μL per well at 4° C. overnight. The next day the solution was removed and the plate was washed twice with 350 μL/well TBST. The plate was blocked by adding 200 μL/well blocking buffer (3% BSA/TBST). The plate was placed at 37° C. for 2 h, and then washed twice with 350 μL/well TBST. A series of concentration of standards and samples were added to each well. The plate was placed at room temperature for 2h. The solution was removed and the plate washed twice with 350 μL/well TBST. Goat Anti-human Kappa light chain (HRP) (abcam, ab202549) was diluted with the blocking buffer and added at 100 μL per well. The plate was incubated at room temperature for 1h. Then the plate was washed four times with 350 μL/well TBST. 100 μL of TMB solution A: solution B, 1:1) solution was added in each well and the reaction was placed in dark for 3-10 mins. 50 μL of stopping solution (2M H₂SO₄) was added and the optical density at 450 nm and 630 nm was read. Data were analyzed with GraphPadPrism5 software.

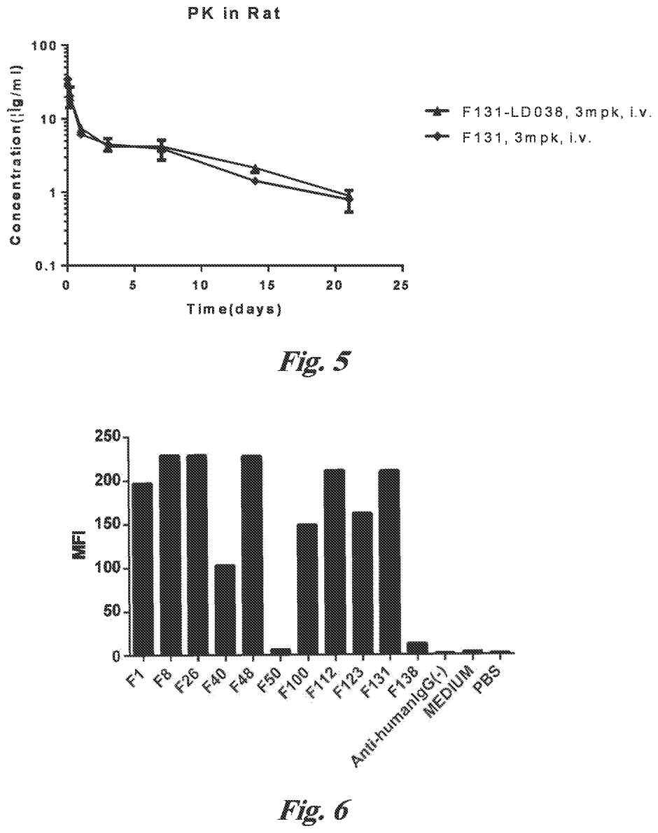

After a single iv administration of anti-huFOLR-1 conjugate F131-LD038 or naked antibody F131 at 3 mg/kg, the PK profile of anti-huFOLR-1 conjugate F131-LD038 was similar to that of naked antibody F131. The results are shown in FIG. 5.

### Example 67. Generation of Human Antibodies Against Human FOLR1

Antibodies targeting human FOLR-1 were screened using a fully human antibody library. This library is a semisynthetic human antibody library in which the Fab was displayed on the surface of phage.

A standard protocol was followed for the library panning. Specifically, PolySorp or MaxiSorp Nunc-Immuno Tubes (Nunc- MG Scientific) were coated with 0.5 ml of human FOLR1 (ACRO-FO1-H52H1) antigen at 6 μg/ml (refer to the panning summary, Table 1), and placed in a refrigerator overnight. The tube was washed once with PBS, blocked with 1% BSA/PBS, and placed at RT (room temperature) for 1 hour. The tube was incubated with the library phage sample at indicated amount (CFU, refer to the panning summary, Table 1) at RT for 1 hour. The tube was washed 10 times with PBST buffer. To elute the bound phage, 0.5 ml of 100 mM TEA (triethylamine) was added, incubated at RT for 2 mins, and the eluate was transferred to a new tube and neutralized immediately by adding 0.25 ml of 1.0 M Tris-HCL, pH 8.0, with mixing. The eluant (0.75 ml) was added into 10 ml of exponentially growing E. coli TG1 (OD600-0.5), mixed well and incubated without shaking at 37° C. (water bath) for 30 min. 10-fold dilutions of the culture were made in 2×TY media and 10 μl of each dilution was plated on TYE/amp/glu plates and incubated at 30° C. overnight. The next day, the colony number for each dilution was counted, and the CFU (colony form unit) for the panning output was calculated. The remaining culture was centrifuged at 2,800 g for 15 min, resuspended in 0.5 ml of 2×TY media, plated on two 150 mm TYE/amp/glu plates, and incubated at 30° C. overnight. The next day, 3-5 ml of 2×TY/amp/glu media was added to each plate and the bacteria were scraped from the plate with a cell spreader. Glycerol stocks were made by mixing 1.5 ml of bacteria and 0.5 ml of 80% glycerol and the stock placed at−80° C.

To prepare phage particles for the next round of selection, the glycerol stocks were inoculated into 40 ml of 2×TY/amp/glu media, starting at OD600-0.01-0.05. The cultures were grown at 37° C. with shaking (300 rpm) until the OD600 reached 0.4-0.6. The cultures were infected by adding helper phage CM13 to the culture at a helper phage:bacteria ratio of 5-10:1. The cultures were incubated at 37° C. for 30 minutes while standing in a water bath with occasional mixing followed by shaking at 37° C. for 30 minutes. The bacterial cultures were centrifuged at 3000 rpm for 20 minutes and the supernatants were removed. The pellets were resuspended in 100 mL of 2×TY/amp/kan and then grown with shaking at 30° C. for overnight. The cultures were harvested by centrifuging at 6,000 g for 30 min. The phage particles were precipitated by adding ⅕ volume of PEG solution into the supernatant followed by 1h incubation on ice followed by centrifuging at 4,000 g for 20 min at 4° C. The supernatants were discarded thoroughly. The phage pellets were resuspended in 1-2 ml of cold PBS. The residual bacteria were removed by micro-centrifugation at top speed for 5 min at 4° C. The phage prepared in this manner can be used immediately for selection, or stored at −80° C. in aliquots with 10% glycerol. The titer of the phage preparations were determined by infecting 100ul of exponentially growing E. coli TG1 with a 10-fold dilution of the phage solution (in 2×TY, down to 10-11). The selection was repeated starting with step 1 for a total round of 3-4 rounds.

A total of 4 rounds of panning were performed. The concentration of the washing buffer PBS-Tween20 in the 2nd, 3rd and 4th rounds was gradually increased to 0.2%, 0.3%, and 0.4%, respectively.

After 4 rounds of screening, the target positive enrichment rate reached 1.5×10⁴, with a significant difference from the blank control, as shown in Table 1. Clones from two 96-well plates were picked for Phage ELISA validation; those clones with high binding with FOLR-1 were selected to sequenced.

In total, sixty-nine clones were sequenced and 12 unique VH sequences were obtained. These 12 VH sequences were analyzed and had 2 unique sets of HCDR3, as showed in Tables 2 and 4. For the clones with the 12 unique VH sequences, the VL sequences were then determined. Two unique VL sequences were obtained with two groups of unique LCDR3, as show in Tables 3 and 4.

Further analysis of the clone sequences using the Kabat system of CDR region showed that clones F1/8/9/26/48/50/100/112/123/131/138 have the same HCDRs and LCDRs, but with different heavy chain frame work (HFR) and light chain frame work (LFR) sequences, as shown in Table 5. Clone F40 has a different HCDRs and LCDRs, and with different HFR and LFR, as shown in Table 5.

TABLE 1
Process monitoring of the 4 rounds panning

| | | | | Enriching |
|---|---|---|---|---|
| Round | Conditions | Input | Output | factor |
| 1^{st} | Target protein: 200 nM (~6 ug/ml) Human FOLR1 | 1.0 × 10¹³ | 2.5 × 10⁵ | 4.0 × 10⁷ |
| | Blocking: 2% M-PBS | | | |
| | Washing: 0.1% Tween20 PBST, 9 times | | | |
| | Elution: TEA | | | |
| | Pre counter select: 2% M-PBS | | | |
| 2^{nd}- | Target protein: 200 nM (~6 ug/ml) Human FOLR1 | 5.3 × 10¹² | 9.6 × 10⁴ | 5.5 × 10⁷ |
| Positive | Blocking: 2% M-PBS | | | |
| Screening | Washing: 0.2% Tween20 PBST, 9 times | | | |
| | Elution: TEA | | | |
| | Pre counter select: 2% M-PBS | | | |
| 2^{nd}- | Target protein: no coating | 6.7 × 10¹¹ | 3.0 × 10⁴ | 2.2 × 10⁷ |
| Negative | Blocking: 2% M-PBS | | | |
| Screening | Washing: 0.2% Tween20 PBST, 9 times | | | |
| | Elution: TEA | | | |
| | Pre counter select: 2% M-PBS | | | |
| 3^{rd}- | Target protein: 200 nM (~6 ug/ml) Human FOLR1 | 5.3 × 10¹² | 3.0 × 10⁷ | 1.8 × 10⁵ |
| Positive | Blocking: 2% M-PBS | | | |
| Screening | Washing: 0.3% Tween20 PBST, 9 times | | | |
| | Elution: TEA | | | |
| | Pre counter select: 2% M-PBS | | | |
| 3^{rd}- | Target protein: no coating | 6.7 × 10¹¹ | 2.1 × 10⁵ | 3.2 × 10⁶ |
| Negative | Blocking: 2% M-PBS | | | |
| Screening | Washing: 0.3% Tween20 PBST, 9 times | | | |
| | Elution: TEA | | | |
| | Pre counter select: 2% M-PBS | | | |
| 4^{th}- | Target protein: 200 nM (~6 ug/ml) Human FOLR1 | 5.1 × 10¹² | 3.3 × 10⁸ | 1.5 × 10⁴ |
| Positive | Blocking: 2% M-PBS | | | |
| Screening | Washing: 0.4% Tween20 PBST, 9 times | | | |
| | Elution: TEA | | | |
| | Pre counter select: 2% M-PBS | | | |
| 4^{th}- | Target protein: no coating | 6.4 × 10¹¹ | 1.4 × 10⁵ | 4.6 × 10⁶ |
| Negative | Blocking: 2% M-PBS | | | |
| Screening | Washing: 0.4% Tween20 PBST, 9 times | | | |
| | Elution: TEA | | | |
| | Pre counter select: 2% M-PBS | | | |

TABLE 2
VH grouping and ranking

| | VH | HCDR3 |
|---|---|---|
| Clones | Grouping | Grouping |
| F8, 24 | VH-1 | HCDR3-A |
| F9 | VH-2 | |
| F26 | VH-3 | |
| F48 | VH-4 | |
| F50 | VH-5 | |
| F100 | VH-6 | |
| F112 | VH-7 | |
| F123 | VH-8 | |
| F131, 139 | VH-9 | |
| F138 | VH-10 | |
| F1, 14, 16, 30, 31, 32, 37, 45, 58, 74, 75, 76, | VH-11 | |
| 77, 78, 81, 82, 86, 87, 88, 89, 92, 93, 95, 96, | | |
| 97, 98, 99, 103, 105, 106, 108, 111, 113, 114, | | |
| 115, 124, 130, 140, 146, 147, 148, 153, 154, | | |
| 162, 163, 164, 169, 170 | | |
| F40 | VH-12 | HCDR3-B |

TABLE 3
VL grouping and ranking

| | VL | LCDR3 |
|---|---|---|
| Clones | Grouping | Grouping |
| F1, 8, 9, 26, 48, 50, 100, 112, 123, 131, 138 | VL-1 | LCDR3-A |
| F40 | VL-2 | LCDR3-B |

TABLE 4
Variable region sequence of anti-FOLR-1 antibodies

| Clone | | |
|---|---|---|
| Seq. | VH | VL |
| F1 | EVQLLESGGGVVQPGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRDNSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 6) | NO: 7) |
| F8 | EVQLLESGGGVVQHGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRDNSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 8) | NO: 9) |
| F9 | EVQLLESGGGVVQLGGPDSPVQPLDSPFS | EIVMTQSPSSVSASVGDRVAITCRASQGI |
| | SYGMHWVRQAPGKGLEWVAVISYDGSNK | SSWLAWYQQKPGKAPKLLIYAASSLQSG |
| | YYADSVKGRFTISRDNSKNTLYLQMNSLRA | VPSRFSGSGSGTDFTLTISSLQPEDFATY |
| | EDTAVYYCARPRAYYGAYGSSFDYWGQG | YCQQSYSTPLTFGGGTKVDIK (SEQ ID |
| | TQVTVSS (SEQ ID NO: 10) | NO: 11) |
| F26 | EVQLLESGGGVVQRGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRDNSKNTLYLPMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 12) | NO: 13) |
| F40 | EVQLLESGGGVVQPGRSLRLSCAASGFTF | DIQVTQSPSSLSASLGDTVSITCRASRGL |
| | SSYAMHWVRQAPGKGLEWVAVISYDGSN | TDSVAWYQQKPGQAPKLLIYAASTLQSG |
| | KYYADSVKGRFIISRDNSKNTVYLQMNSLR | VPSRFGGSGSGSYFTLTITSLQPEDVATY |
| | AEDTAVYYCARPTYVFTYTGSSFDYWGQG | YCQNYKSAPWTFGQGTKVEIK (SEQ ID |
| | TQVTVSS (SEQ ID NO: 14) | NO: 15) |
| F48 | EVQLLESGGGVVQPGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRDNSKNTLYLHMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 16) | NO: 17) |
| F50 | EVQLLESGGGVVQRGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRDNSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 18) | NO: 19) |
| F100 | EVQLLESGGGVVQPGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRPNSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 20) | NO: 21) |
| F112 | EVQLLESGGGVVQPGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRHNSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 22) | NO: 23) |
| F123 | EVQLLESGGGVVQPERSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRANSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 24) | NO: 25) |
| F131 | EVQLLESGGGVVQPGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISRANSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 26) | NO: 27) |
| F138 | EVQLLESGGGVVQPGRSLRLSCAASGFTF | EIVMTQSPSSVSASVGDRVAITCRASQGI |
| | SSYGMHWVRQAPGKGLEWVAVISYDGSN | SSWLAWYQQKPGKAPKLLIYAASSLQSG |
| | KYYADSVKGRFTISTHNSKNTLYLQMNSLR | VPSRFSGSGSGTDFTLTISSLQPEDFATY |
| | AEDTAVYYCARPRAYYGAYGSSFDYWGQ | YCQQSYSTPLTFGGGTKVDIK (SEQ ID |
| | GTQVTVSS (SEQ ID NO: 28) | NO: 29) |

TABLE 5
CDR sequence of anti-FOLR-1 antibodies of the present invention

| Clone | | | | | | |
|---|---|---|---|---|---|---|
| Seq. | HCDR1 | HCDR2 | HCDR3 | LCDR1 | LCDR2 | LCDR3 |
| F1 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F8 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F9 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F26 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F40 | SYAMH | VISYDGSNKYYADSVKG | PTYVFTYTGSSFDY | RASRGLTDSVA | AASTLQS | QNYKSAPW |
| | (SEQ | (SEQ ID NO: 31) | (SEQ ID NO: 37) | (SEQ ID NO: | (SEQ ID | (SEQ ID NO: |
| | ID NO: | | | 38) | NO: 39) | 40) |
| | 36) | | | | | |
| F48 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F50 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F100 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F112 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F123 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F131 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |
| F138 | SYGMH | VISYDGSNKYYADSVKG | PRAYYGAYGSSFDY | RASQGISSWLA | AASSLQS | QQSYSTPLT |
| | (SEQ | (SEQ ID NO: 31) | (SEQ ID NO: 32) | (SEQ ID NO: | (SEQ ID | (SEQ ID NO: |
| | ID NO: | | | 33) | NO: 34) | 35) |
| | 30) | | | | | |

### Example 68. Validation of Antibodies Produced by HEK293 Cells

After obtaining the sequences of the antibody clones (as described above), further analyses were done using the full IgG molecule of the. First, expression of full-length antibody molecules with an IgG1 Fc was performed in 48-well or 96-well microplates, and the supernatants were collected for the detection of expression levels and antigen or cell binding ability.

### 68.1 Antibody Expression in 48 or 96 Wells Plate

The cDNA sequences encoding the heavy and light chains of antibodies F1, F8, F26, F40, F48, F50, F100, F112, F123, F131, and F138 were constructed to the vector PTT5. HEK293 cells were collected, adjusted to a cell density with 1×106/ml, and plated into 48/96-well cell culture plates at 200 or 400 μL per well in a 37° C. incubator with 5% CO2 for later use. For transfection in 96-well plates, 0.5 μg of plasmid was diluted in 20 μL OPTI medium, mixed well, and 2.5 μL of transfection reagent T1 (plasmid: T1=1:5) was diluted in 20 μL OPTI medium, mixed well, incubated at room temperature for 5 min. The transfection reagent T1 diluent was added to the DNA, mixed well, and incubated at room temperature for 30 min. The transfection complex was formed during the incubation. The transfection complex was added to the cells, mixed well, and incubated at 37° C. in a 5% CO2 incubator for 48 hours. When transfecting in 48-well plates, the amount of plasmid and transfection reagent was doubled. On the second day after transfection, the supernatant was collected to detect the antibody bio-activity with ELISA or FACS.

### 68.2 IgG Expression Level.

Antibody expression levels in 96-well were tested by standard ELISA. Briefly, anti-Human IgG Fc antibody (Sigma, 18885-2ML) was diluted to 5 μg/ml with a carbonic acid coating solution at pH 9.6 and 100 μL was coated in each well of 96-well microtiter plate at 4° C. overnight. The liquid in the wells was discarded and the wells were washed three times with PBST, blocked with 4% skimmed milk powder-PBS (Sigma, D5652-1L), 300 μL/well, and incubated in 37° C. for 1 hour. The liquid in the wells was discarded, then the wells were washed with three times with PBS. Samples were added to the 96-well microtiter plate using 100 μL/well. PBS was added in the control group. The plates were incubated at 37° C. for 1 hour, then the liquid was discarded and the wells were washed with three times with PBST. HRP-goat anti-human IgG (Sigma, 118885-2ML) was added (1:5000 dilution) using 100 μL/well and the plates incubated at 37° C. for 1 hour. Then the liquid in the plates was discarded, and the plates were washed five times with PBST. A TMB solution was added using 100 μL/well. 2M H₂SO₄ was then added to each well using 50 μL per well to terminate the reaction after 10-15 mins. The A450 values were read using a microplate reader. The results are shown in Table 6. All of the antibodies had a normal expression except clone F50.

### 68.3 Antibody Binding to Human and Cynomolgus FOLR1 Protein.

The ability of the antibodies to bind to human FOLR1 protein or to cross binding to cynomolgus FOLR1 protein was tested by standard ELISA. Briefly, the human FOLR1 (ACRO-FO1-H52H1) or cynomolgus FOLR1 protein (ACRO, F01-C52H8) with a His tag was diluted to 5 μg/ml with a carbonic acid coating solution at pH 9.6 and 100 μL antigen was coated in each well of 96-well microtiter plate at 4° C. overnight. The liquid in the wells was discarded and the wells were washed three times with PBST. The wells were then blocked with 4% skim milk powder-PBS (Sigma, D5652-1 L), using 300 μL/well and the plates incubated in 37° C. for 1 hour. The liquid in the wells was discarded, and the well were washed three times with PBS. Samples were added using 100 μL/well; PBS was added in the control group. The plates were incubated at 37° C. for 1 hour. The liquid in the wells was discarded and the wells were washed three times with PBST. HRP-goat anti-human IgG (Sigma, 118885-2ML) was added (with 1:5000 dilution, 100 μL/well) and the plates were incubated in 37° C. for 1 hour. Then the liquid in the wells was discarded, and the wells were washed five times with PBST. A TMB solution was added using 100 μL/well. 2M H₂SO₄ was added to each well using 50 μL to terminate the reaction after 10-15 mins. The A450 values were read using a microplate reader.

The expression levels of the IgGs in microtiter plates and the binding to human FOLR1 protein are shown in Table 6. All of the antibodies had a normal binding to human FOLR1 protein except clone F50.

The results of anti-FOLR1 antibody cross binding to cynomolgus FOLR1 protein is shown in Table 7. All of the antibodies had good cross binding to cynomolgus FOLR1 protein except clone F50.

### 68.4 Antibody Binding to Tumor Cell Lines Expressing High FOLR1.

The binding activity of the antibodies to Hela cells (ATCC® CCL-2, provided by COBIOER) and to RPTEC/TERT1 cells (ATCC® CRL-4031, provided by COBIOER) was tested by flow cytometry using the transfection supernatants. Briefly, target cells were digested with 0.02% EDTA-2Na, centrifuged at 1500 rpm for 3 mins, and resuspended with PBS. After counting, the cells were added to a 1.5 ml centrifuge tube with 1×10⁶ cells per tube, centrifuged at 1500 rpm for 5 minutes, and the supernatant was discarded. Then all the operations were carried out on an ice bath. 100 μL of transfection supernatant was added to each 1.5 ml centrifuge tube. Blank cells, blank cells plus secondary antibody, medium and HEK293 supernatant were set up as the controls. The reaction was performed on an ice bath for 1 hour. Then the cells were pelleted and washed twice with PBS. The secondary antibody, goat anti-Human IgG (PE, abcam, ab98596), was diluted (1:200) and added using 100 μL per tube. The reaction was performed on an ice bath for 1 hour in the dark. The cells were pelleted again and washed twice with PBS, resuspended in 300 μL PBS, and FL2 fluorescence readings were measured by cytometry. The results were analyzed by FlowJoTM10 software.

The results for anti-FOLR1 antibody binding to Hela cells are shown in FIG. 6. The results demonstrate that clone F50 was negative for Hela cell binding. The clones F40 and F138 were weakly positive for Hela cell binding. The remaining eight clones were positive for Hela cell binding.

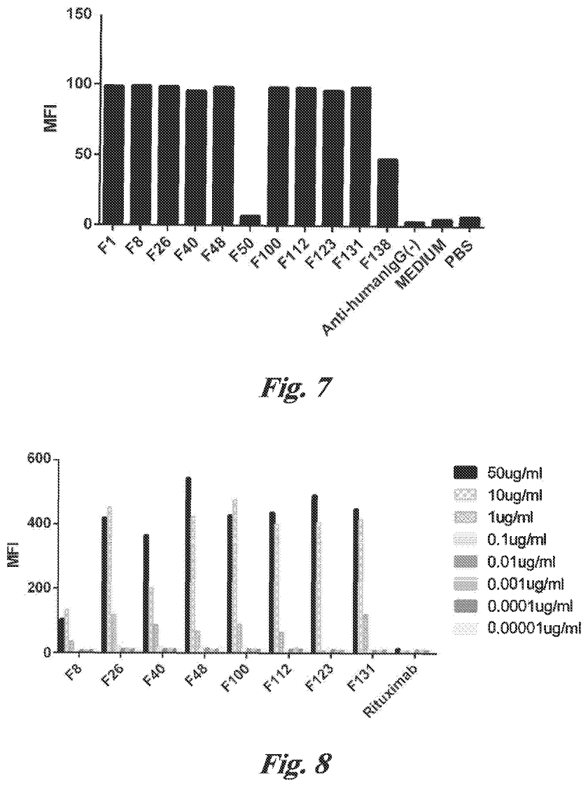

The results of anti-FOLR1 antibody binding to RPTEC/TERT1 cells are shown in FIG. 7. The results demonstrate that clone F50 was negative for RPTEC/TERT1 cell binding. Clone F138 was weakly positive for RPTEC/TERT1 cell binding. The remaining 9 clones were positive for RPTEC/TERT1 cell binding.

TABLE 6
Comparison of antibody levels and
binding to human FOLR1 protein

| | OD450 value | | |
|---|---|---|---|
| | Target binding, | | Target |
| | coating with Human | IgG level, coating | binding/ |
| Samples | FOLR1 Protein | Anti-Human IgG Fc | IgG level |
| F40 | 2.3860 | 2.3350 | 1.022 |
| F123 | 2.3160 | 2.1930 | 1.056 |
| F1 | 2.4330 | 2.2540 | 1.079 |
| F8 | 2.4540 | 2.1900 | 1.121 |
| F131 | 2.3470 | 2.3090 | 1.016 |
| F50 | 0.1300 | 0.4930 | 0.264 |
| F112 | 2.3530 | 2.1010 | 1.120 |
| F48 | 2.5450 | 2.2740 | 1.119 |
| F26 | 2.4310 | 2.0100 | 1.209 |
| F100 | 2.2980 | 1.5550 | 1.478 |
| F138 | 2.4080 | 2.0060 | 1.200 |
| Medium | 0.0920 | 0.1050 | 0.876 |
| PBS | 0.0920 | 0.1430 | 0.643 |

TABLE 7
Comparison of antibody cross binding
ability to cynomolgus FOLR1 protein

| | OD450 value | | | | | |
|---|---|---|---|---|---|---|
| | | | | Target: Cynomolgus/Rhesus | | |
| | Target: Human FOLR1 | | | macaque FOLR1, Protein, | | |
| | Protein His Tag | | | His Tag | | |
| | Diluted | Diluted | Diluted | Diluted | Diluted | Diluted |
| Samples | 1x | 5x | 25x | 1x | 5x | 25x |
| F40 | 0.811 | 0.382 | 0.163 | 1.354 | 1.111 | 0.924 |
| F123 | 1.715 | 1.468 | 1.328 | 1.363 | 1.301 | 1.074 |
| F1 | 1.660 | 1.445 | 1.177 | 1.384 | 1.256 | 0.994 |
| F8 | 1.672 | 1.645 | 1.315 | 0.896 | 1.285 | 1.059 |
| F131 | 1.609 | 1.522 | 1.384 | 1.203 | 1.270 | 1.088 |
| F50 | 0.094 | 0.129 | 0.098 | 0.093 | 0.107 | 0.066 |
| F112 | 1.556 | 1.421 | 1.174 | 1.257 | 1.212 | 0.933 |
| F48 | 1.557 | 1.590 | 1.290 | 1.148 | 1.040 | 0.908 |
| F26 | 1.638 | 1.515 | 1.475 | 1.237 | 1.207 | 1.018 |
| F100 | 1.609 | 1.528 | 1.180 | 1.163 | 1.034 | 0.875 |
| F138 | 1.636 | 1.692 | 1.827 | 1.140 | 1.142 | 1.173 |
| Medium | 0.085 | 0.092 | 0.081 | 0.170 | 0.151 | 0.096 |

| | | |
|---|---|---|
| PBS | 0.082 | 0.081 |

### Example 69. Characterization of Anti-Human FOLR1 Antibodies Produced by HEK293 Cell Expression in Shaker Flask

The binding of the anti-FOLR1 antibodies was quantitatively studied by obtaining a sufficient amount of protein by expressing the antibodies in suspension cells. The plasmids were transfected into the suspension cells for expression. The supernatants were collected for antibody purification. Highly purity antibodies were used to quantitatively detect the binding and internalization of the antibody on tumor cells that had high FOLR1 protein levels.

### 69.1 Antibody Expression and Purification.

The plasmids encoding antibodies F8, F26, F40, F48, F100, F112, F123, and F131 were transfected into HEK293 cells. Briefly, HEK293 cells were collected, adjusted to a cell density with 1×10⁶/ml and cultured with 30 mL medium in 125 mL shaker flasks in a 37° C. shaker with 5% CO₂ for later use. For transfection, 30 μg of plasmid was diluted in 1500ul KPM medium, mixed well, and 150 μL of transfection reagent T1 (plasmid:T1=1:5) was diluted in 1500 μL KPM medium, mixed well and incubated at room temperature for 5 min. The transfection reagent T1 diluent was added to the DNA, mixed well, and incubated at room temperature for 30 mins to form the transfection complex. The transfection complex was added to the cells, mixed well, and incubated at 37° C. in a 5% CO2 shaker at 120 rpm for 48 hours. TN1 solution was added to a final concentration of 0.5% after 24h. On the sixth day after transfection, the supernatant was collected and purified.

Antibody purification was carried out by a standard process using protein A or protein G. Briefly, each supernatant was filtered through a 0.22 μm filter membrane and loaded onto column equilibrated with binding buffer (PB, pH7.2). The column was washed with binding buffer until a stable baseline was obtained with no absorbance at 280 nm. Antibody was eluted with 0.1M citric acid buffer containing 0.15M NaCl, pH3.4, using a flow rate of 1 ml/min. Fractions of approximately 1.5-3.5 ml were collected and neutralized by the addition of 10% volume of 1M Tris-HCl, pH 9.0. Then the antibody samples were dialyzed overnight twice against 1×PBS and sterilized by filtering through a 0.2 μm filter membrane. The purity was tested using 12% SDS-PAGE.

The expression levels and purification result are shown in Table 8. Antibodies F8, F26 and F131 had higher expression levels while antibody F100 had the lowest expression level. All the antibodies had a high purity (data not shown).

TABLE 8
Comparison of antibody expression levels

| Host cell | Clone | Con. (ug/ml) | Vol.(ml) | Qua. (mg) |
|---|---|---|---|---|
| HEK293 | F8 | 387.6 | 4.00 | 1.55 |
| HEK293 | F26 | 396.6 | 3.50 | 1.39 |
| HEK293 | F40 | 182.1 | 4.01 | 0.73 |
| HEK293 | F48 | 131.1 | 2.97 | 0.39 |
| HEK293 | F100 | 60.6 | 1.98 | 0.12 |
| HEK293 | F112 | 129.6 | 4.01 | 0.52 |
| HEK293 | F123 | 171.6 | 2.97 | 0.51 |
| HEK293 | F131 | 359.1 | 3.51 | 1.26 |

### 69.2 Antibody Binding to Tumor Cell Lines Having High FOLR1 Levels.

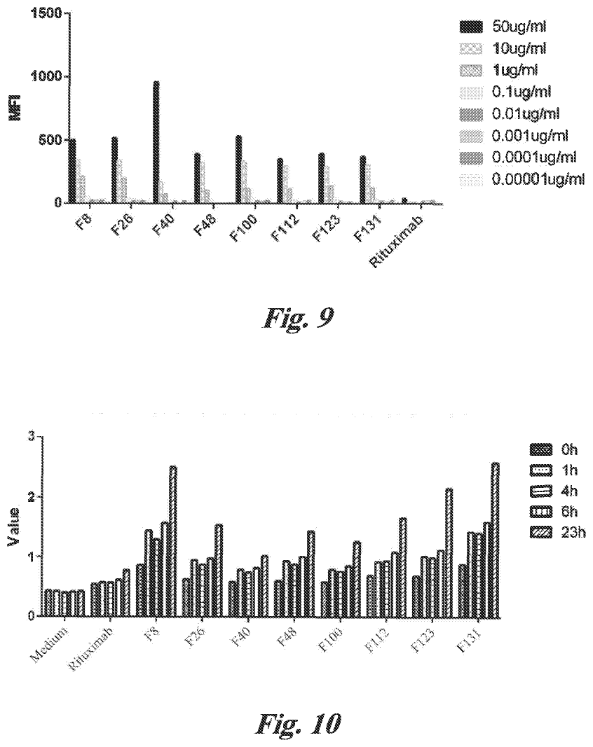

Anti-FOLR1 antibody binding to Hela and RPTEC/TERT1 cells was tested by FACS. The study was carried out as described above. The results are shown in FIGS. 8 and 9. All antibodies bind to Hela and to RPTEC/TERT1 cells in a dose-dependent.

### 69.3 Internalization Rate Characterization.

Anti-FOLR1 antibodies F8, F26, F40, F48, F100, F112, F123, and F131 were tested for the ability to internalize into the FOLR-1-expressing tumor cell lines Hela and RPTEC/TERT1 using a pHAb assay where the antibodies were labelled with pHAb fluorescent dye. Antibody labeling was performed according to the directions in the kit. Specifically, 50 μL of magnetic beads were added to a 1.5 ml EP tube. The EP tube was placed on a magnetic stand for 10s and the protective solution over the magnetic beads was removed. Each tube of magnetic beads was washed with 250 μL PB and 100 μg antibody was added to each tube of magnetic beads (buffer system: citric acid/sodium Tris-HCl (pH 6.0)). The volume was made up to 1 ml with PB, and the reaction solution was mixed and rotated for 1 h at room temperature. Then the magnetic beads were washed with 250 μL PB, equilibrated with 250 μL NaHCO₃. 100 μL NaHCO₃ and 1.2 μL of prepared pHAb dye (prepared before use) was added to each tube and the reaction was placed for 1 h in the dark. Each tube was washed twice with 250 μL PB. 50 mM glycine was added to each tube using 100 μL at room temperature for 5 min and then labeled antibody was eluted. Then 2M Tris buffer was added to the elution for neutralization. The final labeled antibody was stored in the dark for later use.

Hela or RPTEC/TERT1 cells were seeded at 15,000 cells per well with 100 μL and cultured in a 5% CO2 incubator at 37° C. for 20-24 h. pHAb-labeled test antibodies were added to the wells at a concentration of 10 μg/ml. The plates were then read on a Thermo VARIOSKAN FLASH with an excitation wavelength of 520 nm and an absorption wavelength of 570 nm at 0 h, 1 h, 4 h, 6 h, and 23 h, respectively.

The results are shown in FIG. 10 and FIG. 11. All the anti-FOLR1 antibodies tested showed a time dependent increase in pHAb fluorescence in FOLR1-expressing Hela and RPTEC/TERT1 cells. This result indicates that each antibody internalized into Hela and RPTEC/TERT1 cells, with antibodies F8 and F131 having the most strongly internalization rate.

### Example 70. Characterization of Anti-FOLR-1 Immunoconjugates

Further characterization of the anti-FOLR-1 antibodies as immunoconjugates was performed.

### 70.1 Expression of Reference Antibody and Antibodies F8, F26 and F131

The ImmunoGen Inc. anti-FOLR-1 antibody mirvetuximab (huFR107) was used as a control. The amino acid sequences of the VH and VL regions of huFR107 were obtained from U.S. Pat. No. 8,557,966 (SEQ ID NOs:36 and 37, respectively) and were codon-optimized. The optimized cDNAs encoding huFR107 and encoding antibodies F8, F26 and F131 were constructed in the vector pcDNA3.4. Then the plasmids were transiently transected into ExpiCHO-S cells using a standard ExpiFectamine CHO Transfection procedure (Gibco, A29129) in Erlenmeyer flasks. The suspended transient transfections were incubated for 10 days and then the cleared supernatants were purified by a Protein A column and followed by SDS-PAGE as described above.

### 70.2 Preparation of Anti-FOLR-1 Immunoconjugates

The pH of antibody solution was adjusted within the range of pH 7.0-7.5 by adding 0.5M sodium phosphate dibasic. The indicated amount of 0.5M EDTA was added to achieve a final EDTA concentration of 5 mM in the antibody solution. The indicated amount of 10 mM TCEP (Tris(2-chloroethyl) phosphate solution was added to achieve the desired TCEP/mAb molar ratio. The reduction reaction was placed at RT for 90 mins. Then DMSO was added to achieve a 10% v/v. The drug-linker mc-VC-PAB-MMAE was dissolved in DMSO to achieve a final concentration of 10 mM and the indicated amount was added in the reaction solution in a molar excess of 30-50% compared to the moles of cysteine thiols available. The conjugation reaction was placed at RT for 30 mins. NAC (N-Acetyl-L-cysteine) stock solution was added to achieve an NAC/Mc-VC-PAB-MMAE molar ratio of 5. The quenched reaction was placed at RT for 15 mins. The purification was carried out by PD10 column.

The purity of the anti-FOLR-1 immunoconjugates was assessed with size exclusion chromatography (SEC) on a TSK gel G3000SWXL, 7.8×300 mm column (Tosoh Bioscience) using the Waters HPLC E2695&2489 system. The operation was carried out at 25° C., using a mobile phase of 50 mM Na₂PO₄ (pH 6.7) and 10% IPA, run with a flow rate of 0.8 mL/min over 20 min. Referring to Table 9, all four ADCs had high purity.

The hydrophobicity of the anti-FOLR-1 immunoconjugates were assessed with Hydrophobic interaction chromatography (HIC) on a Hydrophobic interaction TosoHaas TSK gel Butyl-NPR column (4.6 mm ID×3.5 cm., with a particle size of 2.5 μm) using the Waters HPLC E2695&2489 system. Briefly, the HPLC system was operated at 25° C. with mobile phase A:50 mM Na₂PO₄ /1.5 M (NH₄)2SO₄ pH 7.0 and mobile phase B: 50 mM Na₂PO₄/25% IPA, pH 7.0. The mobile phases were filtered through a 0.22-μm membrane filter (Millipore), run with a flow rate of 0.5 mL, 30 min. The parameters of the linear gradient are shown in Table 10. The DARs (Drug antibody ratios) of the anti-FOLR-1 immunoconjugates were determined according to the HIC data and were within the range of 3-4 (data not shown).

TABLE 9
Purity of anti-FOLR1 immunoconjugates

| | F8-ADC | F26-ADC | F131-ADC | FR107-ADC |
|---|---|---|---|---|
| Purity (%) | 98% | 100% | 100% | 100% |

TABLE 10
Process for the linear gradient

| Time/min | B/100% |
|---|---|
| 0.0 | 0 |
| 12.0 | 100 |
| 12.1 | 0 |
| 18.0 | 0 |

### Example 71. Affinity Data of F131 and F131-LD038 to FOLR Family Proteins Tested by BLI

Recombinant proteins consisting of FOLR family proteins' extracellular domain linked to His tag were either purchased (from ACRO systems) or synthesized in house. For binding studies via biolayer interferometry (BLI), F131 (at 16.67 nM) was immobilized on anti-human IgG Fc biosensor tips (Fortebio). Binding assays using varying concentration (from 500 nM down to 7.8 nM) of recombinant antigen proteins in solution were performed using Octet RED (Fortebio). Association time was set at 180s and dissociation time was set at 300s. Binding affinity was calculated using ForteBio Data Acquisition 6.3 software (ForteBio), and affinity was derived by fitting the kinetic data to a 1:1 Langmuir binding model utilizing global fitting algorithms. F131 displayed high affinity to human FOLR1, while having low response to human FOLR2, and no response to human FOLR3, demonstrating binding specificity of F131 (Table 11). F131 demonstrated high bing affinity to human and cynomolgus monkey FOLR1 with an equilibrium dissociation constant (KD) of 1.5 and 8.1 nM, respectively. F131 displayed no cross-reactivity to rat FOLR1, and low cross-reactivity to mouse FOLR1 (KD=2.9 μM).

TABLE 11
Affinity data of F131 and F131-LD038 to FOLR family proteins tested by BLI

| | Loading | Loading | | | | | | |
|---|---|---|---|---|---|---|---|---|
| | Sample | Conc. | Conc. | | | | | |
| Ag | ID | (ug/ml) | (nM) | Response | KD (M) | ka (1/Ms) | kdis (1/s) | Full R\hat{ }2 |
| Hu- | F131 mAb | 2.5 | 500. | 0.1924 | 5.296E−09 | 2.261E05 | 1.198E−03 | 0.9978 |
| FRα | | | | | | | | |
| Hu- | F131- | 2.5 | 500. | 0.2224 | 4.792E−09 | 2.519E05 | 1.207E−03 | 0.9977 |
| FRα | LD038 | | | | | | | |
| Hu- | F131 mAb | 2.5 | 250. | 0.1782 | 5.296E−09 | 2.261E05 | 1.198E−03 | 0.9978 |
| FRα | | | | | | | | |
| Hu- | F131- | 2.5 | 250. | 0.2008 | 4.792E−09 | 2.519E05 | 1.207E−03 | 0.9977 |
| FRα | LD038 | | | | | | | |
| Hu- | F131 mAb | 2.5 | 125. | 0.1444 | 5.296E−09 | 2.261E05 | 1.198E−03 | 0.9978 |
| FRα | | | | | | | | |
| Hu- | F131- | 2.5 | 125. | 0.176 | 4.792E−09 | 2.519E05 | 1.207E−03 | 0.9977 |
| FRα | LD038 | | | | | | | |
| Hu- | F131 mAb | 2.5 | 62.5 | 0.1172 | 5.296E−09 | 2.261E05 | 1.198E−03 | 0.9978 |
| FRα | | | | | | | | |
| Hu- | F131- | 2.5 | 62.5 | 0.1378 | 4.792E−09 | 2.519E05 | 1.207E−03 | 0.9977 |
| FRα | LD038 | | | | | | | |
| Hu- | F131 mAb | 2.5 | 31.3 | 0.0766 | 5.296E−09 | 2.261E05 | 1.198E−03 | 0.9978 |
| FRα | | | | | | | | |
| Hu- | F131- | 2.5 | 31.3 | 0.092 | 4.792E−09 | 2.519E05 | 1.207E−03 | 0.9977 |
| FRα | LD038 | | | | | | | |
| Hu- | F131 mAb | 2.5 | 15.6 | 0.0447 | 5.296E−09 | 2.261E05 | 1.198E−03 | 0.9978 |
| FRα | | | | | | | | |
| Hu- | F131- | 2.5 | 15.6 | 0.0564 | 4.792E−09 | 2.519E05 | 1.207E−03 | 0.9977 |
| FRα | LD038 | | | | | | | |
| Hu- | F131 mAb | 2.5 | 7.82 | 0.0259 | 5.296E−09 | 2.261E05 | 1.198E−03 | 0.9978 |
| FRα | | | | | | | | |
| Hu- | F131- | 2.5 | 7.82 | 0.0267 | 4.792E−09 | 2.519E05 | 1.207E−03 | 0.9977 |
| FRα | LD038 | | | | | | | |
| Hu- | F131 mAb | 2.5 | 500. | 0.037 | 1.420E−07 | 5.524E05 | 7.843E−02 | 0.9083 |
| FRb | | | | | | | | |
| Hu- | F131- | 2.5 | 500. | 0.0427 | 8.442E−08 | 9.631E05 | 8.130E−02 | 0.9153 |
| FRb | LD038 | | | | | | | |
| Hu- | F131 mAb | 2.5 | 250. | 0.0207 | 1.420E−07 | 5.524E05 | 7.843E−02 | 0.9083 |
| FRb | | | | | | | | |
| Hu- | F131- | 2.5 | 250. | 0.0243 | 8.442E−08 | 9.631E05 | 8.130E−02 | 0.9153 |
| FRb | LD038 | | | | | | | |
| Hu- | F131 mAb | 2.5 | 125. | 0.0188 | 1.420E−07 | 5.524E05 | 7.843E−02 | 0.9083 |
| FRb | | | | | | | | |
| Hu- | F131- | 2.5 | 125. | 0.0161 | 8.442E−08 | 9.631E05 | 8.130E−02 | 0.9153 |
| FRb | LD038 | | | | | | | |
| Hu- | F131 mAb | 2.5 | 62.5 | 0.0356 | 1.420E−07 | 5.524E05 | 7.843E−02 | 0.9083 |
| FRb | | | | | | | | |
| Hu- | F131- | 2.5 | 62.5 | 0.0379 | 8.442E−08 | 9.631E05 | 8.130E−02 | 0.9153 |
| FRb | LD038 | | | | | | | |
| Hu- | F131 mAb | 2.5 | 31.3 | *0.0068 | 1.420E−07 | 5.524E05 | 7.843E−02 | 0.9083 |
| FRb | | | | | | | | |
| Hu- | F131- | 2.5 | 31.3 | *0.0076 | 8.442E−08 | 9.631E05 | 8.130E−02 | 0.9153 |
| FRb | LD038 | | | | | | | |
| Hu- | F131 mAb | 2.5 | 15.6 | *0.002 | 1.420E−07 | 5.524E05 | 7.843E−02 | 0.9083 |
| FRb | | | | | | | | |
| Hu- | F131- | 2.5 | 15.6 | *0.0041 | 8.442E−08 | 9.631E05 | 8.130E−02 | 0.9153 |
| FRb | LD038 | | | | | | | |
| Hu- | F131 mAb | 2.5 | 7.82 | *0.0011 | 1.420E−07 | 5.524E05 | 7.843E−02 | 0.9083 |
| FRb | | | | | | | | |
| Hu- | F131- | 2.5 | 7.82 | *0.002 | 8.442E−08 | 9.631E05 | 8.130E−02 | 0.9153 |
| FRb | LD038 | | | | | | | |
| Hu- | F131 mAb | 2.5 | 500. | *0.0024 | 1.306E−04 | 1.663E03 | 2.173E−01 | 0.1554 |
| FRg | | | | | | | | |
| Hu- | F131- | 2.5 | 500. | *0.0096 | 3.284E−11 | 7.096E09 | 2.330E−01 | 0.7567 |
| FRg | LD038 | | | | | | | |
| Hu- | F131 mAb | 2.5 | 250. | *0.0003 | 1.306E−04 | 1.663E03 | 2.173E−01 | 0.1554 |
| FRg | | | | | | | | |
| Hu- | F131- | 2.5 | 250. | *0.0051 | 3.284E−11 | 7.096E09 | 2.330E−01 | 0.7567 |
| FRg | LD038 | | | | | | | |
| Hu- | F131 mAb | 2.5 | 125. | *0.0051 | 1.306E−04 | 1.663E03 | 2.173E−01 | 0.1554 |
| FRg | | | | | | | | |
| Hu- | F131- | 2.5 | 125. | *0.009 | 3.284E−11 | 7.096E09 | 2.330E−01 | 0.7567 |
| FRg | LD038 | | | | | | | |
| Hu- | F131 mAb | 2.5 | 62.5 | *−2.028E−03 | 1.306E−04 | 1.663E03 | 2.173E−01 | 0.1554 |
| FRg | | | | | | | | |
| Hu- | F131- | 2.5 | 62.5 | *0.007 | 3.284E−11 | 7.096E09 | 2.330E−01 | 0.7567 |
| FRg | LD038 | | | | | | | |
| Hu- | F131 mAb | 2.5 | 31.3 | *0.0028 | 1.306E−04 | 1.663E03 | 2.173E−01 | 0.1554 |
| FRg | | | | | | | | |
| Hu- | F131- | 2.5 | 31.3 | *0.0079 | 3.284E−11 | 7.096E09 | 2.330E−01 | 0.7567 |
| FRg | LD038 | | | | | | | |
| Hu- | F131 mAb | 2.5 | 15.6 | *−2.611E−03 | 1.306E−04 | 1.663E03 | 2.173E−01 | 0.1554 |
| FRg | | | | | | | | |
| Hu- | F131- | 2.5 | 15.6 | *0.0084 | 3.284E−11 | 7.096E09 | 2.330E−01 | 0.7567 |
| FRg | LD038 | | | | | | | |
| Hu- | F131 mAb | 2.5 | 7.82 | *1.025E−03 | 1.306E−04 | 1.663E03 | 2.173E−01 | 0.1554 |
| FRg | | | | | | | | |
| Hu- | F131- | 2.5 | 7.82 | *0.0004 | 3.284E−11 | 7.096E09 | 2.330E−01 | 0.7567 |
| FRg | LD038 | | | | | | | |

*Response below range of quantification

TABLE 12
Affinity data of F131 and F131-LD038 to species FOLR α proteins tested by BLI

| | Loading | Loading | | | | | | |
|---|---|---|---|---|---|---|---|---|
| | Sample | Conc. | Conc. | | | | | |
| Ag | ID | (ug/ml) | (nM) | Response | KD (M) | ka (1/Ms) | kdis (1/s) | Full R\hat{ }2 |
| Hu-Ag | F131 | 2.5 | 500. | 0.181 | 1.528E−09 | 2.210E05 | 3.377E−04 | 0.9875 |
| | mAb | | | | | | | |
| Hu-Ag | F131- | 2.5 | 500. | 0.2049 | 4.801E−09 | 2.236E05 | 1.073E−03 | 0.9488 |
| | LD038 | | | | | | | |
| Hu-Ag | F131 | 2.5 | 250. | 0.1617 | 1.528E−09 | 2.210E05 | 3.377E−04 | 0.9875 |
| | mAb | | | | | | | |
| Hu-Ag | F131- | 2.5 | 250. | 0.1843 | 4.801E−09 | 2.236E05 | 1.073E−03 | 0.9488 |
| | LD038 | | | | | | | |
| Hu-Ag | F131 | 2.5 | 125. | 0.1458 | 1.528E−09 | 2.210E05 | 3.377E−04 | 0.9875 |
| | mAb | | | | | | | |
| Hu-Ag | F131- | 2.5 | 125. | 0.1577 | 4.801E−09 | 2.236E05 | 1.073E−03 | 0.9488 |
| | LD038 | | | | | | | |
| Hu-Ag | F131 | 2.5 | 62.5 | 0.1202 | 1.528E−09 | 2.210E05 | 3.377E−04 | 0.9875 |
| | mAb | | | | | | | |
| Hu-Ag | F131- | 2.5 | 62.5 | 0.1292 | 4.801E−09 | 2.236E05 | 1.073E−03 | 0.9488 |
| | LD038 | | | | | | | |
| Hu-Ag | F131 | 2.5 | 31.3 | 0.0869 | 1.528E−09 | 2.210E05 | 3.377E−04 | 0.9875 |
| | mAb | | | | | | | |
| Hu-Ag | F131- | 2.5 | 31.3 | 0.0866 | 4.801E−09 | 2.236E05 | 1.073E−03 | 0.9488 |
| | LD038 | | | | | | | |
| Hu-Ag | F131 | 2.5 | 15.6 | 0.0503 | 1.528E−09 | 2.210E05 | 3.377E−04 | 0.9875 |
| | mAb | | | | | | | |
| Hu-Ag | F131- | 2.5 | 15.6 | 0.0447 | 4.801E−09 | 2.236E05 | 1.073E−03 | 0.9488 |
| | LD038 | | | | | | | |
| Hu-Ag | F131 | 2.5 | 7.82 | 0.04 | 1.528E−09 | 2.210E05 | 3.377E−04 | 0.9875 |
| | mAb | | | | | | | |
| Hu-Ag | F131- | 2.5 | 7.82 | 0.0342 | 4.801E−09 | 2.236E05 | 1.073E−03 | 0.9488 |
| | LD038 | | | | | | | |
| Cyno- | F131 | 2.5 | 500. | 0.2191 | 8.148E−09 | 1.929E05 | 1.572E−03 | 0.9864 |
| Ag | mAb | | | | | | | |
| Cyno- | F131- | 2.5 | 500. | 0.1858 | 8.823E−09 | 1.810E05 | 1.597E−03 | 0.993 |
| Ag | LD038 | | | | | | | |
| Cyno- | F131 | 2.5 | 250. | 0.209 | 8.148E−09 | 1.929E05 | 1.572E−03 | 0.9864 |
| Ag | mAb | | | | | | | |
| Cyno- | F131- | 2.5 | 250. | 0.1799 | 8.823E−09 | 1.810E05 | 1.597E−03 | 0.993 |
| Ag | LD038 | | | | | | | |
| Cyno- | F131 | 2.5 | 125. | 0.1775 | 8.148E−09 | 1.929E05 | 1.572E−03 | 0.9864 |
| Ag | mAb | | | | | | | |
| Cyno- | F131- | 2.5 | 125. | 0.152 | 8.823E−09 | 1.810E05 | 1.597E−03 | 0.993 |
| Ag | LD038 | | | | | | | |
| Cyno- | F131 | 2.5 | 62.5 | 0.14 | 8.148E−09 | 1.929E05 | 1.572E−03 | 0.9864 |
| Ag | mAb | | | | | | | |
| Cyno- | F131- | 2.5 | 62.5 | 0.1211 | 8.823E−09 | 1.810E05 | 1.597E−03 | 0.993 |
| Ag | LD038 | | | | | | | |
| Cyno- | F131 | 2.5 | 31.3 | 0.084 | 8.148E−09 | 1.929E05 | 1.572E−03 | 0.9864 |
| Ag | mAb | | | | | | | |
| Cyno- | F131- | 2.5 | 31.3 | 0.0702 | 8.823E−09 | 1.810E05 | 1.597E−03 | 0.993 |
| Ag | LD038 | | | | | | | |
| Cyno- | F131 | 2.5 | 15.6 | 0.0485 | 8.148E−09 | 1.929E05 | 1.572E−03 | 0.9864 |
| Ag | mAb | | | | | | | |
| Cyno- | F131- | 2.5 | 15.6 | 0.0384 | 8.823E−09 | 1.810E05 | 1.597E−03 | 0.993 |
| Ag | LD038 | | | | | | | |
| Cyno- | F131 | 2.5 | 7.82 | 0.0537 | 8.148E−09 | 1.929E05 | 1.572E−03 | 0.9864 |
| Ag | mAb | | | | | | | |
| Cyno- | F131- | 2.5 | 7.82 | 0.0225 | 8.823E−09 | 1.810E05 | 1.597E−03 | 0.993 |
| Ag | LD038 | | | | | | | |
| Rat-Ag | F131 | 2.5 | 500. | *0.0037 | 1.874E−04 | 5.494E03 | 1.030E00 | |
| | mAb | | | | | | | |
| Rat-Ag | F131- | 2.5 | 500. | *0.0036 | 8.481E−08 | 2.635E06 | 2.235E−01 | |
| | LD038 | | | | | | | |
| Rat-Ag | F131 | 2.5 | 250. | *−9.448E−04 | 1.874E−04 | 5.494E03 | 1.030E00 | |
| | mAb | | | | | | | |
| Rat-Ag | F131- | 2.5 | 250. | *−1.028E−03 | 8.481E−08 | 2.635E06 | 2.235E−01 | |
| | LD038 | | | | | | | |
| Rat-Ag | F131 | 2.5 | 125. | *−4.774E−03 | 1.874E−04 | 5.494E03 | 1.030E00 | |
| | mAb | | | | | | | |
| Rat-Ag | F131- | 2.5 | 125. | *0.0007 | 8.481E−08 | 2.635E06 | 2.235E−01 | |
| | LD038 | | | | | | | |
| Rat-Ag | F131 | 2.5 | 62.5 | *−6.060E−03 | 1.874E−04 | 5.494E03 | 1.030E00 | |
| | mAb | | | | | | | |
| Rat-Ag | F131- | 2.5 | 62.5 | *−3.930E−03 | 8.481E−08 | 2.635E06 | 2.235E−01 | |
| | LD038 | | | | | | | |
| Rat-Ag | F131 | 2.5 | 31.3 | *−5.315E−03 | 1.874E−04 | 5.494E03 | 1.030E00 | |
| | mAb | | | | | | | |
| Rat-Ag | F131- | 2.5 | 31.3 | *−4.536E−03 | 8.481E−08 | 2.635E06 | 2.235E−01 | |
| | LD038 | | | | | | | |
| Rat-Ag | F131 | 2.5 | 15.6 | *−3.949E−03 | 1.874E−04 | 5.494E03 | 1.030E00 | |
| | mAb | | | | | | | |
| Rat-Ag | F131- | 2.5 | 15.6 | *−2.122E−03 | 8.481E−08 | 2.635E06 | 2.235E−01 | |
| | LD038 | | | | | | | |
| Rat-Ag | F131 | 2.5 | 7.82 | *−5.629E−03 | 1.874E−04 | 5.494E03 | 1.030E00 | |
| | mAb | | | | | | | |
| Rat-Ag | F131- | 2.5 | 7.82 | *−4.383E−03 | 8.481E−08 | 2.635E06 | 2.235E−01 | |
| | LD038 | | | | | | | |
| Mouse- | F131 | 2.5 | 500. | 0.0167 | 2.957E−06 | 1.019E05 | 3.012E−01 | 0.6807 |
| Ag | mAb | | | | | | | |
| Mouse- | F131- | 2.5 | 500. | 0.0125 | 2.872E−06 | 9.263E04 | 2.660E−01 | 0.4778 |
| Ag | LD038 | | | | | | | |
| Mouse- | F131 | 2.5 | 250. | *0.008 | 2.957E−06 | 1.019E05 | 3.012E−01 | 0.6807 |
| Ag | mAb | | | | | | | |
| Mouse- | F131- | 2.5 | 250. | *0.0013 | 2.872E−06 | 9.263E04 | 2.660E−01 | 0.4778 |
| Ag | LD038 | | | | | | | |
| Mouse- | F131 | 2.5 | 125. | *−5.791E−04 | 2.957E−06 | 1.019E05 | 3.012E−01 | 0.6807 |
| Ag | mAb | | | | | | | |
| Mouse- | F131- | 2.5 | 125. | *0.0008 | 2.872E−06 | 9.263E04 | 2.660E−01 | 0.4778 |
| Ag | LD038 | | | | | | | |
| Mouse- | F131 | 2.5 | 62.5 | *−1.782E−03 | 2.957E−06 | 1.019E05 | 3.012E−01 | 0.6807 |
| Ag | mAb | | | | | | | |
| Mouse- | F131- | 2.5 | 62.5 | *−2.420E−04 | 2.872E−06 | 9.263E04 | 2.660E−01 | 0.4778 |
| Ag | LD038 | | | | | | | |
| Mouse- | F131 | 2.5 | 31.3 | *−2.515E−03 | 2.957E−06 | 1.019E05 | 3.012E−01 | 0.6807 |
| Ag | mAb | | | | | | | |
| Mouse- | F131- | 2.5 | 31.3 | *−1.320E−03 | 2.872E−06 | 9.263E04 | 2.660E−01 | 0.4778 |
| Ag | LD038 | | | | | | | |
| Mouse- | F131 | 2.5 | 15.6 | *−5.099E−03 | 2.957E−06 | 1.019E05 | 3.012E−01 | 0.6807 |
| Ag | mAb | | | | | | | |
| Mouse- | F131- | 2.5 | 15.6 | *−3.244E−03 | 2.872E−06 | 9.263E04 | 2.660E−01 | 0.4778 |
| Ag | LD038 | | | | | | | |
| Mouse- | F131 | 2.5 | 7.82 | *0.0027 | 2.957E−06 | 1.019E05 | 3.012E−01 | 0.6807 |
| Ag | mAb | | | | | | | |
| Mouse- | F131- | 2.5 | 7.82 | *−4.834E−03 | 2.872E−06 | 9.263E04 | 2.660E−01 | 0.4778 |
| Ag | LD038 | | | | | | | |

*Response below range of quantification

### Example 72. F131 and F131-LD038 Internalization in Tumor Cell Lines

An internalization assay was conducted in a time course. 3×10⁵ Cells were incubated for 30 min at 4° C. with 10 ug/ml of F131 or F131-LD038 in FACS buffer (1×PBS containing 0.1% BSA). Cells were washed at 4° C. to remove unbound material and kept on ice or shifted to 37° C. as needed. At progressive time points (0, 0.5, 1, 2, 3, 4h), cells were stained with PE-conjugated anti-human Fc for 30 min at 4° C. and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time 0, then divided by the MFI of cell surface-bound antibody at 4° C. at time 0.

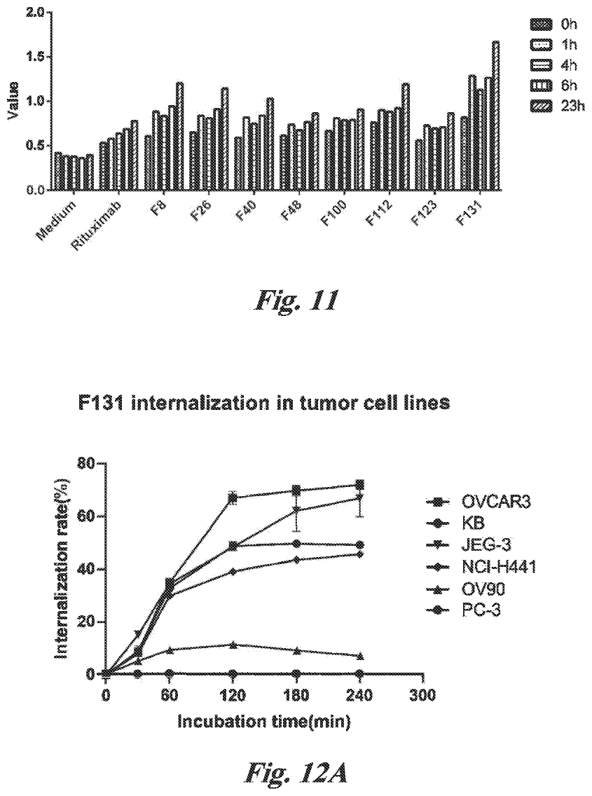

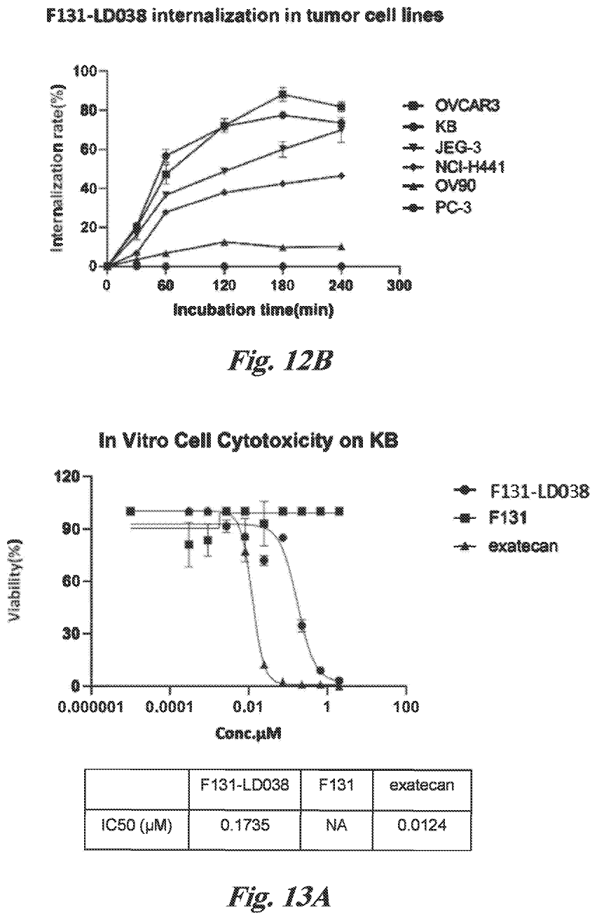

F131 and F131-LD038 displayed rapid internalization on multiple FOLRα-expressing cell lines (OVCAR3, KB, JEG-3, NCI-H441) while no internalization was observed on FOLRα non-expressing cells (PC-3, control) (FIG. 12A and FIG. 12B).

### Example 73. F131 and F131-LD038 Cytotoxicity in Three Select Cell Lines

One day prior to adding test article (F131 or F131-LD038 or exatecan), cells were harvested and plated into 96-well solid white flat bottom plates. The next day cells were exposed to the test article at concentrations from 2000 to 0.305 nM for F131 and F131-LD038, or from 100 nM to 1 μM for exatecan. Plates were incubated at 37° C. for 96h. After that, 40 μl Cell-titre Glo (CTG) per well was added to the plates with luciferase readings collected at 5 min after and analyzed by Microplate readers. All readings were normalized as percentage of viable cells in the untreated control wells and the IC50 values were calculated by Prism software.

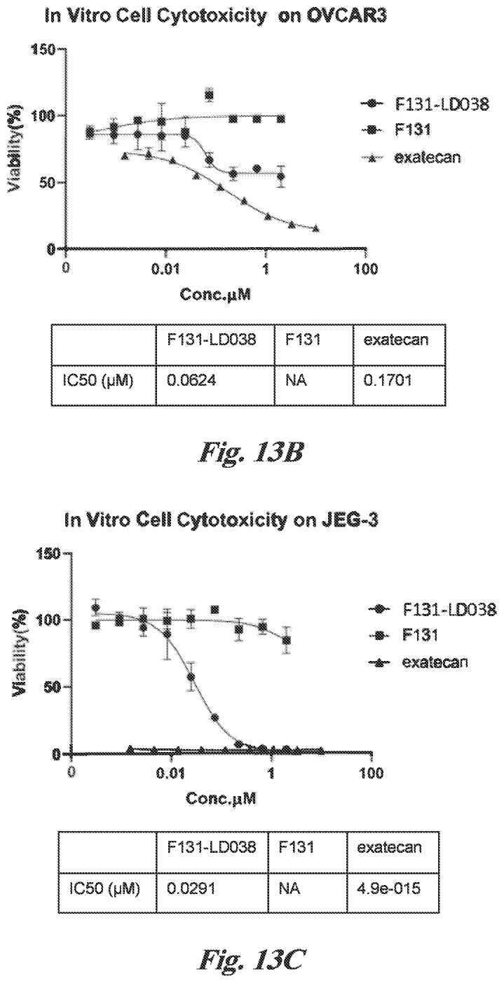

F131-LD038 produced strong cytotoxicity on human FOLRα-expressing KB, OVCAR3 and JEG-3 cells, while naked antibody F131 exerted no cytotoxicity on these target tumor cells. The payload (exatecan) of F131-LD038 appeared to be more potent than F131-LD038 in cytotoxic effects on these cells (FIGS. 13A-13C).

### Example 74. F131-LD038 In Vivo Efficacy in CDX

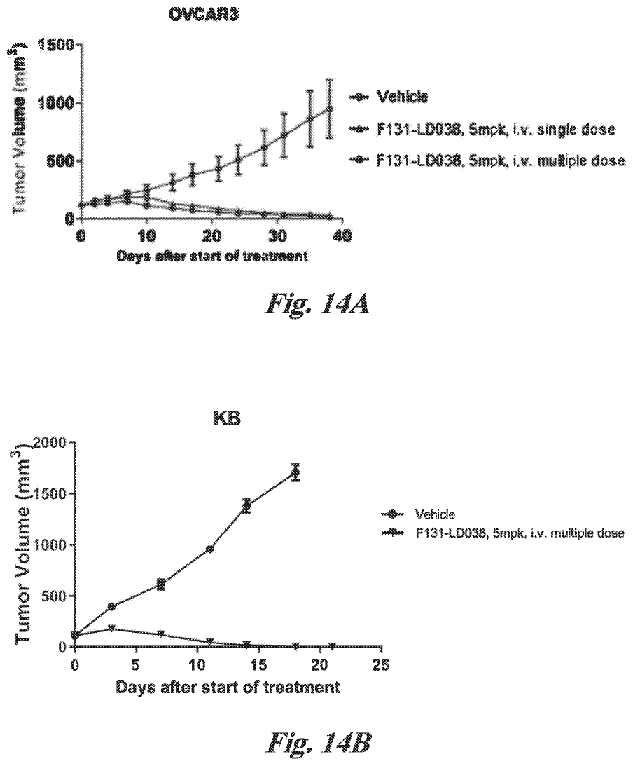

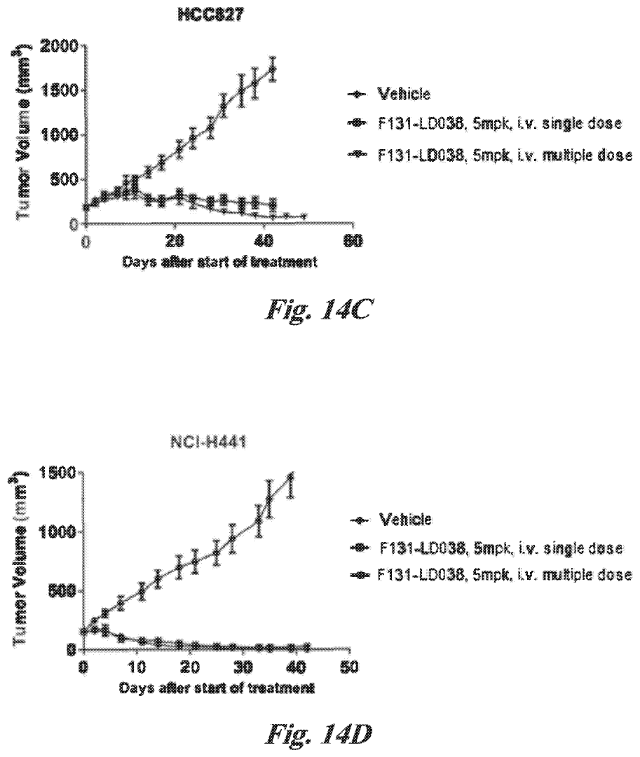

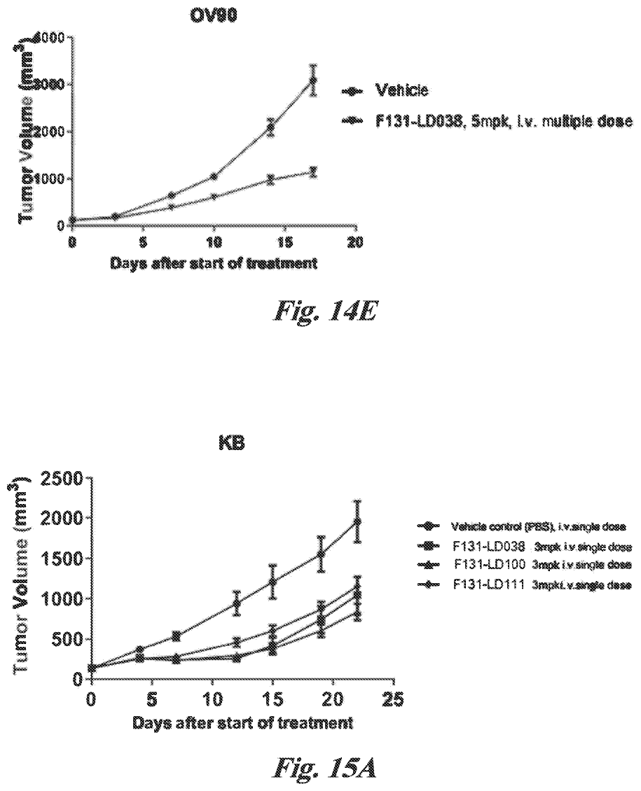

The in vivo anti-tumor activity of F131-LD038 ADC was evaluated in cell lines that have a range of target expression levels, such as NCI-H441, HCC827, OVCAR3, KB and OV90 (FIGS. 14A-14E).

NCI-H441 (ATCC, HTB-174, NSCLC, 47*10³ copies per cell) tumor model was established by injecting 5×10⁶ cells suspended in Matrigel/medium (1:1); HCC827 (ATCC, Cat #CRL-2868™, NSCLC, 41*10³ copy per cell) tumor model was established by injecting 4×10⁶ cells suspended in Matrigel/medium (1:1); OVCAR3 (ATCC, HTB-161, Ovarian, 290*10³ copy per cell) tumor model was established by injecting 1×10⁷ cells suspended in Matrigel/medium (1:1); KB (Co-bioer, Oral epithelial, 341*10³ copy per cell) tumor model was established by injecting 1×10⁷ cells suspended in 0.1 mL medium; OV90 (Truway-bio, Ovarian, 0.38*10³ copy per cell) tumor model was established by injecting 2×10⁶ cells suspended in Matrigel/medium (1:1).

6 to 26 days after tumor inoculation, mice with average tumor size of 110-180 mm³ were selected and assigned into 2 or 3 groups for each model using stratified randomization (n=6-9 per group) based upon their tumor volumes. The treatments started one day after randomization (randomization day defined as DO) and were in either a single-dose (on day1) or multiple-dose (on day1/day4/day8/day11) regimens, via intravenous injection of F131-LD038 at 5 mg/kg.

The tumor size and body weight were measured twice a week in two dimensions using a caliper, and the volume was expressed in mm³ using the formula: V=0.5a×b² where a and b are the long and short diameters of the tumor, respectively. Tumor volume exceeding 2000 mm³ was defined as reaching the end of the experiment. Animal body weight was monitored as an indirect measure of tolerability. No mice showed significant weight loss in any of the treatment groups. There were no morbidity and deaths during the treatment duration.

Compared to vehicle control group, all the treatment groups with F131-LD038 at single and multiple dose produced a significant antitumor activity in NCI-H441, HCC827, OVCAR3, KB tumor model, while F131-LD038 displayed a moderate to weak antitumor activity in OV90 tumor model of which the target density is low than on the other tumors.

The target copy number was tested by QIFKIT(DAKO, K0078). Briefly, Cells were labeled with primary mouse monoclonal antibody directed against the antigen of interest. The cells, Set-Up Beads, and Calibration Beads of the kit were then labeled in parallel with fluorescein-conjugated anti-mouse secondary antibody. The fluorescence correlated with the number of bound primary antibody molecules on the cells and on the beads. The samples were then analyzed on the flow cytometer and copy number calculated based on the equation derived from the calibration curve (Table 13).

TABLE 13

| Cell line | Tumor type | Copy number |
|---|---|---|
| OVCAR-3 | ovarian | 290291 |
| KB | oral epithelial | 341364 |
| HCC827 | NSCLC | 40919 |
| NCI-H441 | NSCLC | 46889 |
| OV90 | ovarian | 380 |

### Example 75. In Vivo Efficacy of F131-LD038 and Other F131-Conjugates

75.1 Generation of F131-LD100, F131-LD111, F131-LD101, F131-LD110:

### F131-LD100

2 mL of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb of 8.0. The reducing reaction was conducted for 2 hours at 25° C. The excess TCEP and its byproduct were removed by ultrafiltration with pH=6.9, 50 mM sodium phosphate buffer. Drug linker LD100 (salt of TFA) was dissolved in water at a concentration of 20 mg/mL and added to the reduced mAb at a molar ratio of 8.5 (LD100/mAb). The coupling reaction was stirred for 2 hours at 25° C. The excess LD100 and its impurities were removed by ultrafiltration with 50 mM sodium phosphate buffer. The ADC was stored in 20 mM histidine buffer containing 6% sucrose and 0.02% (w/V) Tween 20 by UFDF. The purity of ADC as determined by SEC-HPLC was 97.1% and DAR value as determined by LC-MS was 7.9.

### F131-LD111

2 mL of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb of 8.0. The reducing reaction was conducted for 2 hours at 25° C. The excess TCEP and its byproduct were removed by ultrafiltration with pH=6.9 50 mM sodium phosphate buffer. Drug linker LD111 (salt of TFA) was dissolved in water at a concentration of 20 mg/mL and added to the reduced mAb at a molar ratio of 8.5 (LD111/mAb). The coupling reaction was stirred for 2 hours at 25° C. The excess LD111 and its impurities were removed by ultrafiltration with 50 mM sodium phosphate buffer. The ADC was stored in 20 mM histidine buffer containing 6% sucrose and 0.02% (w/V) Tween 20 by UFDF. The purity of ADC as determined by SEC-HPLC was 97.5% and DAR value as determined by LC-MS was 7.8.

### F131-LD101

2 mL of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb of 8.0. The reducing reaction was conducted for 2 hours at 25° C. The excess TCEP and its byproduct were removed by ultrafiltration with pH=6.9 50 mM sodium phosphate buffer. Drug linker LD101 (salt of TFA) was dissolved in water at a concentration of 20 mg/mL and added to reduced mAb at a molar ratio of 8.5 (LD101/mAb). The coupling reaction was stirred for 2 hours at 25° C. The excess LD101 and its impurities were removed by ultrafiltration with 50 mM sodium phosphate buffer. The ADC was stored in 20 mM histidine buffer containing 6% sucrose and 0.02% (w/V) Tween 20 by UFDF. The purity of ADC as determined by SEC-HPLC was 98.0% and DAR value as determined by LC-MS was 7.5.

### F131-LD110

2 ml of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb of 8.0. The reducing reaction was conducted for 2 hours at 25° C. The excess TCEP and its byproduct were removed by ultrafiltration with pH=6.9 50 mM sodium phosphate buffer. Drug linker LD110 (salt of TFA) was dissolved in water at a concentration of 20 mg/mL and added to reduced mAb at a molar ratio of 7.7 (LD110/mAb). The coupling reaction was stirred for 2 hours at 25° C. The excess LD110 and its impurities were removed by ultrafiltration with 50 mM sodium phosphate buffer. The ADC was stored in 20 mM histidine buffer containing 6% sucrose and 0.02% (w/V) Tween 20 by UFDF. The purity of ADC as determined by SEC-HPLC was 97.8% and DAR value as determined by LC-MS was 7.5.

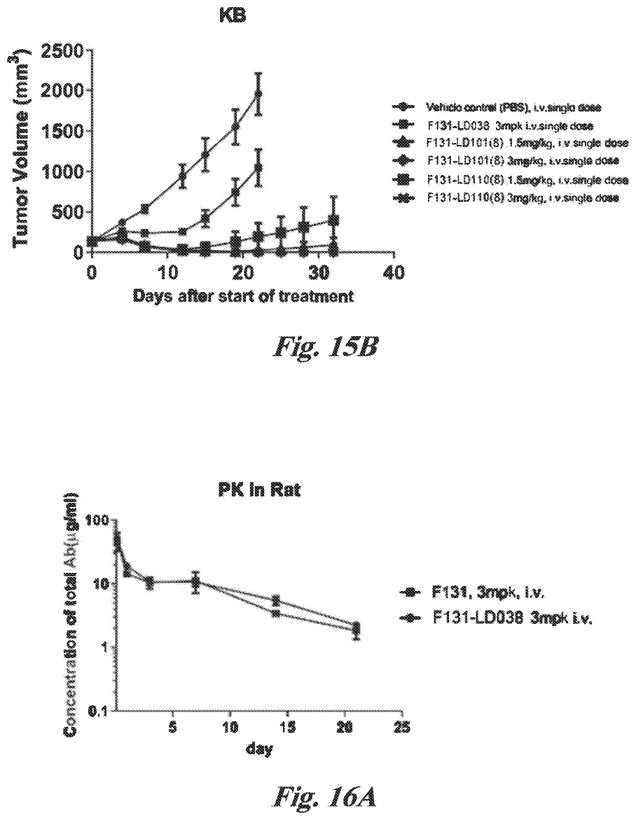

75.2 the In Vivo Anti-Tumor Activity of Additional F131 Conjugates(F131-LD100, F131-LD111, F131-LD101, F131-LD110) were Evaluated in Tumor Cell Line KB (FIGS. 15A and 15B).

A KB(Co-bioer) tumor model was established by injecting 1×10⁷ cells suspended in 0.1 mL medium. 12 days after tumor inoculation, mice with average tumor size−137 mm³ were selected and assigned into 8 groups using stratified randomization (n=8 per group) based upon their tumor volumes. The treatments started one day after randomization (randomization day defined as DO) and the mice were treated with a single (on day1) intravenous injection of F131-LD100 at 3 mg/kg, or F131-LD111 at 3 mg/kg, or F131-LD101 at 1.5 or 3 mg/kg, or F131-LD110 at 1.5 or 3 mg/kg.

The tumor size and body weight were measured as described before. Animal body weight was monitored as an indirect measure of tolerability. No mice showed significant weight loss in any of the treatment groups. There were no morbidity and deaths during the treatment duration.

Compared to vehicle control group, treatment with F131-LD101 and F131-LD110 at 1.5 or 3 mg/kg produced a significant antitumor activity in KB tumor model (FIG. 15B). Tumor growth-inhibition by F131-LD110 was dose-related. Treatment with F131-LD100, F131-LD111, or F131-LD038 at 3 mg/kg produced moderate antitumor activity in KB tumor (FIG. 15A).

TABLE 14

| Drug | LD | ADC (DAR) |
|---|---|---|
| exatecan | LD038 (“PB038”) | F131-LD038(8) |
| exatecan | LD100 (“PB100”) | F131-LD100(8) |
| exatecan | LD111 (“PB111”) | F131-LD111(8) |
| MMAE | LD101 (“PB101”) | F131-LD101(8) |
| MMAE | LD110 (“PB110”) | F131-LD110(8) |

### Example 76. F131-LD038 PK Study in Rat Model of F131, F131-LD038, and Other Conjugates

F131 and its conjugates (F131-LD038, F131-LD101, F131-LD110, F131-LD100, F131-LD111) were intravenously administered at 3 mg/kg to male Sprague Dawley rats (n=3 per group). Orbital blood was cross-sampled from each rat at 10 min, 4h, 1d, 4d, 7d, 10d, 14d, and 21d post dosing. Total Ab concentration (representing F131 and its conjugates) in serum was detected by an ELISA kit generated by Genscript and calculated using Winnonlin 8.2 software.

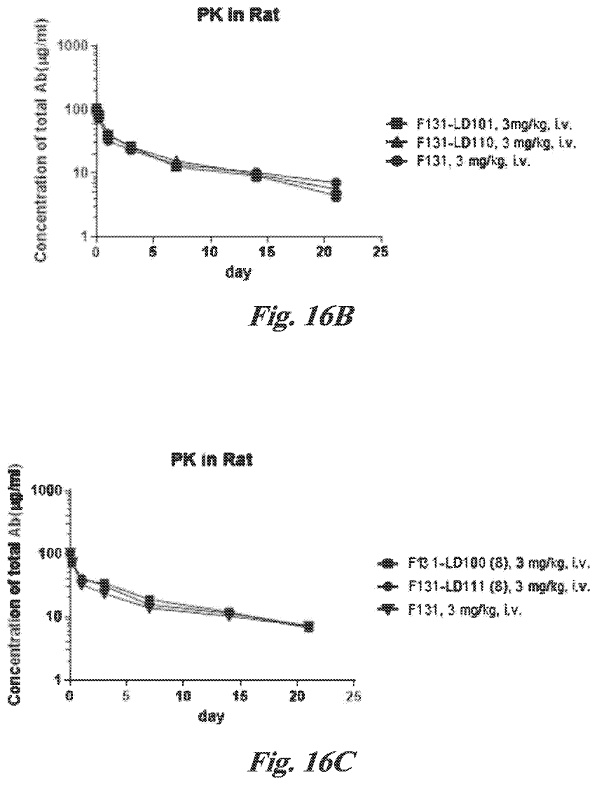

All of the F131-conjugates tested exhibited excellent PK characteristics that are comparable to the unconjugated parental mAb, F131 (FIGS. 16A-16C).

### Example 77. F131-LD038 PK and Tolerability in the Pilot Cynomolgus Toxicity Study

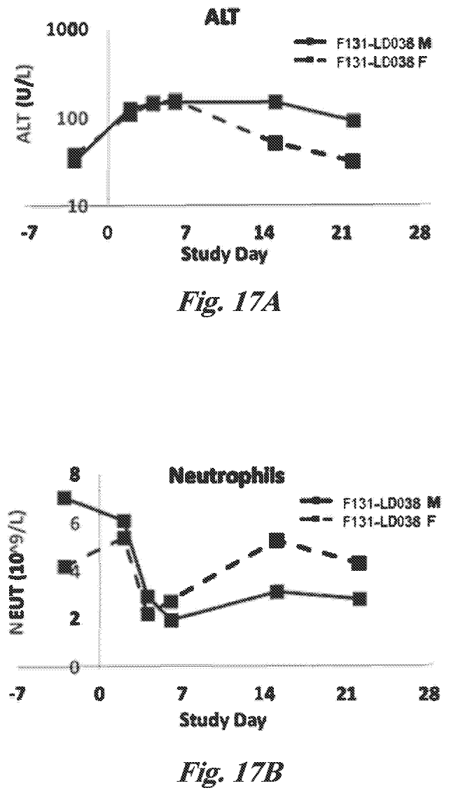

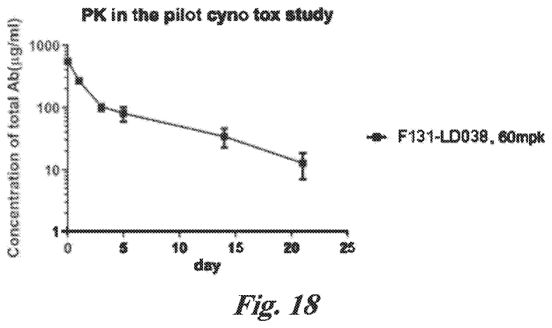

To evaluate the toxicity effect and toxicokinetic characteristics of F131-LD038, F131-LD038 was intravenously administered at a single dose at 60 mg/kg to one male (M) and one female (F) cynomolgus monkeys. Clinical signs, body weight, food consumption, and clinical pathology were monitored throughout the study (Table 15). Animals were euthanized on day 22 post-dosing, upon which a complete necropsy was performed. Toxicokinetic samples were collected from each animal at Oh, 24h, 72h, 120h, 336h, and 504h post dosing. Total Ab concentration (representing F131 and its conjugate) in serum were detected by an ELISA kit generated by Genscript and calculated using Winnonlin 8.2 software. Transient increase in ALT (FIG. 17A) and transient reduction in neutrophils (FIG. 17B) were observed. F131-LD038 displayed a stable and excellent plasma PK profile in cyno (FIG. 18).

TABLE 15

| | Clinical | | Clinical |
|---|---|---|---|
| Test Article | Observations | Hematology | Chemistry |
| F131-LD038 | Feces soft | ↓: WBC; | ↑: ALT; |
| (60 mg/kg) | Appetite | NEUT; | AST |
| (one male; | lessens | RET/RET % | ↓: TG |
| one female) | | | |

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including International Patent Application No. PCT/CN2021/104618, filed Jul. 6, 2021, and Chinese Patent Application No. 202210777240.7, filed Jul. 4, 2022, are incorporated herein by reference, in their entirety. Aspects of the embodiments can be modified, if necessary to employ concepts of the various patents, applications and publications to provide yet further embodiments.

Various publications, including patents, patent application publications, and scientific literature, are cited herein, the disclosures of which are incorporated by reference in their entireties for all purposes.

SEQUENCE LISTING

SEQ ID NO: 1
(GGGGS) n, where n = 1 to 5
SEQ ID NO: 2 human IgG1 heavy chain UniProt P01857-1
ASTKGPSVFP LAPSSKSTSG GTAALGCLVK DYFPEPVTVS WNSGALTSGV
HTFPAVLQSS GLYSLSSVVT VPSSSLGTQT YICNVNHKPS NTKVDKKVEP
KSCDKTHTCP PCPAPELLGG PSVFLFPPKP KDTLMISRTP EVTCVVVDVS
HEDPEVKFNW YVDGVEVHNA KTKPREEQYN STYRVVSVLT VLHQDWLNGK
EYKCKVSNKA LPAPIEKTIS KAKGQPREPQ VYTLPPSRDE LTKNQVSLTC
LVKGFYPSDI AVEWESNGQP ENNYKTTPPV LDSDGSFFLY SKLTVDKSRW
QQGNVFSCSV MHEALHNHYT QKSLSLSPGK
SEQ ID NO: 3 human Kappa light chain UniProt P01834-1
RTVAAPSVFI FPPSDEQLKS GTASVVCLLN NFYPREAKVQ WKVDNALQSG
NSQESVTEQD SKDSTYSLSS TLTLSKADYE KHKVYACEVT HQGLSSPVTK
SFNRGEC
SEQ ID NO: 4 hexa-histidine
HHHHHH
SEQ ID NO: 5
LPXTG
SEQ ID NO: 6 F1 VH amino acid sequence
EVQLLESGGGVVQPGRSLRLSCAASGFTFSSYGMHWVRQAPGKGLEWVAVISYDGSNKYYADSVKGRFTISRDNS
KNTLYLQMNSLRAEDTAVYYCARPRAYYGAYGSSFDYWGQGTQVTVSS
SEQ ID NO: 7 F1 VL amino acid sequence
EIVMTQSPSSVSASVGDRVAITCRASQGISSWLAWYQQKPGKAPKLLIYAASSLQSGVPSRFSGSGSGTDETLTI
SSLQPEDFATYYCQQSYSTPLTFGGGTKVDIK
SEQ ID NO: 8 F8 VH amino acid sequence
EVQLLESGGGVVQHGRSLRLSCAASGFTFSSYGMHWVRQAPGKGLEWVAVISYDGSNKYYADSVKGRFTISRDNS
KNTLYLQMNSLRAEDTAVYYCARPRAYYGAYGSSFDYWGQGTQVTVSS
SEQ ID NO: 9 F8 VL amino acid sequence
EIVMTQSPSSVSASVGDRVAITCRASQGISSWLAWYQQKPGKAPKLLIYAASSLQSGVPSRFSGSGSGTDFTLTI
SSLQPEDFATYYCQQSYSTPLTFGGGTKVDIK
SEQ ID NO: 10 F9 VH amino acid sequence
EVQLLESGGGVVQLGGPDSPVQPLDSPFSSYGMHWVRQAPGKGLEWVAVISYDGSNKYYADSVKGRFTISRDNSK
NTLYLQMNSLRAEDTAVYYCARPRAYYGAYGSSFDYWGQGTQVTVSS
SEQ ID NO: 11 F9 VL amino acid sequence
EIVMTQSPSSVSASVGDRVAITCRASQGISSWLAWYQQKPGKAPKLLIYAASSLQSGVPSRFSGSGSGTDETLTI
SSLQPEDFATYYCQQSYSTPLTFGGGTKVDIK
SEQ ID NO: 12 F26 VH amino acid sequence
EVQLLESGGGVVQRGRSLRLSCAASGFTFSSYGMHWVRQAPGKGLEWVAVISYDGSNKYYADSVKGRFTISRDNS
KNTLYLPMNSLRAEDTAVYYCARPRAYYGAYGSSFDYWGQGTQVTVSS
SEQ ID NO: 13 F26 VL amino acid sequence
EIVMTQSPSSVSASVGDRVAITCRASQGISSWLAWYQQKPGKAPKLLIYAASSLQSGVPSRESGSGSGTDETLTI
SSLQPEDFATYYCQQSYSTPLTFGGGTKVDIK
SEQ ID NO: 14 F40 VH amino acid sequence
EVQLLESGGGVVQPGRSLRLSCAASGFTFSSYAMHWVRQAPGKGLEWVAVISYDGSNKYYADSVKGRFIISRDNS
KNTVYLQMNSLRAEDTAVYYCARPTYVFTYTGSSFDYWGQGTQVTVSS
SEQ ID NO: 15 F40 VL amino acid sequence
DIQVTQSPSSLSASLGDTVSITCRASRGLTDSVAWYQQKPGQAPKLLIYAASTLQSGVPSRFGGSGSGSYFTLTI
TSLQPEDVATYYCQNYKSAPWTFGQGTKVEIK
SEQ ID NO: 16 F48 VH amino acid sequence
EVQLLESGGGVVQPGRSLRLSCAASGFTFSSYGMHWVRQAPGKGLEWVAVISYDGSNKYYADSVKGRFTISRDNS
KNTLYLHMNSLRAEDTAVYYCARPRAYYGAYGSSFDYWGQGTQVTVSS
SEQ ID NO: 17 F48 VL amino acid sequence
EIVMTQSPSSVSASVGDRVAITCRASQGISSWLAWYQQKPGKAPKLLIYAASSLQSGVPSRFSGSGSGTDETLTI
SSLQPEDFATYYCQQSYSTPLTFGGGTKVDIK
SEQ ID NO: 18 F50 VH amino acid sequence
EVQLLESGGGVVQRGRSLRLSCAASGFTFSSYGMHWVRQAPGKGLEWVAVISYDGSNKYYADSVKGRFTISRDNS
KNTLYLQMNSLRAEDTAVYYCARPRAYYGAYGSSFDYWGQGTQVTVSS
SEQ ID NO: 19-F50 VL amino acid sequence
EIVMTQSPSSVSASVGDRVAITCRASQGISSWLAWYQQKPGKAPKLLIYAASSLQSGVPSRESGSGSGTDETLTI
SSLQPEDFATYYCQQSYSTPLTFGGGTKVDIK
SEQ ID NO: 20 F100 VH amino acid sequence
EVQLLESGGGVVQPGRSLRLSCAASGFTFSSYGMHWVRQAPGKGLEWVAVISYDGSNKYYADSVKGRFTISRPNS
KNTLYLQMNSLRAEDTAVYYCARPRAYYGAYGSSFDYWGQGTQVTVSS
SEQ ID NO: 21 F100 VL amino acid sequence
EIVMTQSPSSVSASVGDRVAITCRASQGISSWLAWYQQKPGKAPKLLIYAASSLQSGVPSRFSGSGSGTDETLTI
SSLQPEDFATYYCQQSYSTPLTFGGGTKVDIK
SEQ ID NO: 22 F112 VH amino acid sequence
EVQLLESGGGVVQPGRSLRLSCAASGFTFSSYGMHWVRQAPGKGLEWVAVISYDGSNKYYADSVKGRFTISRHNS
KNTLYLQMNSLRAEDTAVYYCARPRAYYGAYGSSFDYWGQGTQVTVSS
SEQ ID NO: 23 F112 VL amino acid sequence
EIVMTQSPSSVSASVGDRVAITCRASQGISSWLAWYQQKPGKAPKLLIYAASSLQSGVPSRESGSGSGTDETLTI
SSLQPEDFATYYCQQSYSTPLTFGGGTKVDIK
SEQ ID NO: 24 F123 VH amino acid sequence
EVQLLESGGGVVQPERSLRLSCAASGFTFSSYGMHWVRQAPGKGLEWVAVISYDGSNKYYADSVKGRFTISRANS
KNTLYLQMNSLRAEDTAVYYCARPRAYYGAYGSSFDYWGQGTQVTVSS
SEQ ID NO: 25 F123 VL amino acid sequence
EIVMTQSPSSVSASVGDRVAITCRASQGISSWLAWYQQKPGKAPKLLIYAASSLQSGVPSRESGSGSGTDETLTI
SSLQPEDFATYYCQQSYSTPLTFGGGTKVDIK
SEQ ID NO: 26 F131 VH amino acid sequence
EVQLLESGGGVVQPGRSLRLSCAASGFTFSSYGMHWVRQAPGKGLEWVAVISYDGSNKYYADSVKGRFTISRANS
KNTLYLQMNSLRAEDTAVYYCARPRAYYGAYGSSFDYWGQGTQVTVSS
SEQ ID NO: 27 F131 VL amino acid sequence
EIVMTQSPSSVSASVGDRVAITCRASQGISSWLAWYQQKPGKAPKLLIYAASSLQSGVPSRESGSGSGTDETLTI
SSLQPEDFATYYCQQSYSTPLTFGGGTKVDIK
SEQ ID NO: 28 F138 VH amino acid sequence
EVQLLESGGGVVQPGRSLRLSCAASGFTFSSYGMHWVRQAPGKGLEWVAVISYDGSNKYYADSVKGRFTISTHNS
KNTLYLQMNSLRAEDTAVYYCARPRAYYGAYGSSFDYWGQGTQVTVSS
SEQ ID NO: 29 F138 VL amino acid sequence
EIVMTQSPSSVSASVGDRVAITCRASQGISSWLAWYQQKPGKAPKLLIYAASSLQSGVPSRESGSGSGTDETLTI
SSLQPEDFATYYCQQSYSTPLTFGGGTKVDIK
SEQ ID NO: 30 HCDR1 amino acid sequence
SYGMH
SEQ ID NO: 31 HCDR2 amino acid sequence
VISYDGSNKYYADSVKG
SEQ ID NO: 32 HCDR3 amino acid sequence
PRAYYGAYGSSFDY
SEQ ID NO: 33 LCDR1 amino acid sequence
RASQGISSWLA
SEQ ID NO: 34 LCDR2 amino acid sequence
AASSLQS
SEQ ID NO: 35 LCDR3 amino acid sequence
QQSYSTPLT
SEQ ID NO: 36 HCDR1 amino acid sequence
SYAMH
SEQ ID NO: 37 HCDR3 amino acid sequence
PTYVFTYTGSSFDY
SEQ ID NO: 38 LCDR1 amino acid sequence
RASRGLTDSVA
SEQ ID NO: 39 LCDR2 amino acid sequence
AASTLQS
SEQ ID NO: 40 LCDR3 amino acid sequence
QNYKSAPW
SEQ ID NO: 41 human IgG1 heavy chain UniProt P01857-1
ASTKGPSVFP LAPSSKSTSG GTAALGCLVK DYFPEPVTVS WNSGALTSGV
HTFPAVLQSS GLYSLSSVVT VPSSSLGTQT YICNVNHKPS NTKVDKKVEP
KSCDKTHTCP PCPAPELLGG PSVFLFPPKP KDTLMISRTP EVTCVVVDVS
HEDPEVKFNW YVDGVEVHNA KTKPREEQYN STYRVVSVLT VLHQDWLNGK
EYKCKVSNKA LPAPIEKTIS KAKGQPREPQ VYTLPPSRDE LTKNQVSLTC
LVKGFYPSDI AVEWESNGQP ENNYKTTPPV LDSDGSFFLY SKLTVDKSRW
QQGNVESCSV MHEALHNHYT QKSLSLSPGK
SEQ ID NO: 42 human Kappa light chain UniProt P01834-1
RTVAAPSVFI FPPSDEQLKS GTASVVCLLN NFYPREAKVQ WKVDNALQSG
NSQESVTEQD SKDSTYSLSS TLTLSKADYE KHKVYACEVT HQGLSSPVTK
SFNRGEC

1. A Drug-Linker, having the following formula (III): 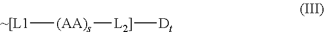
or a salt thereof, wherein:
(i) L1 is a Stretcher unit having an attachment site for a Targeting unit, wherein the wavy (˜) line indicates an attachment site for the Targeting unit;
(ii) AA is an Amino Acid unit having from 1 to 12 subunits;
(iii) s is 0 or 1;
(iv) L2 is a Linker Subunit attached to the Drug unit (D), wherein the Linker Subunit is a cleavable linker unit that comprises a cleavable peptide, and wherein t is 1 to 4;
(v) Drug unit is selected from a cytotoxic agent, an immune modulatory agent, a nucleic acid, a growth inhibitory agent, a PROTAC, a toxin, a radioactive isotope and a chelating ligand; and
(vi) at least one PEG unit,
wherein the at least one PEG unit is present within the Amino Acid unit, the Linker Subunit, or combinations thereof, and wherein the at least one PEG unit has the formula: 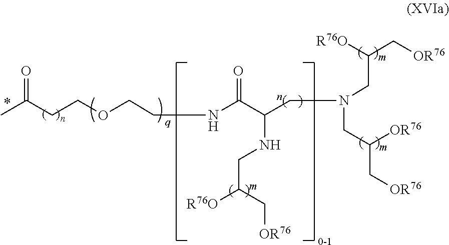
each R⁷⁶ is independently H, acetyl, —P(═O)(OH)₂, or —(CH₂)ᵥS(═O)₂(OH);
each q is independently 1-26;
each m is independently 1 to 4;
each n is independently 1 to 4;
each v is independently 1 to 6; and
each *indicates an attachment site to the Linker Subunit, Amino Acid unit, or both.

2. The Drug-Linker or salt of claim 1, wherein the at least one PEG unit has the formula:

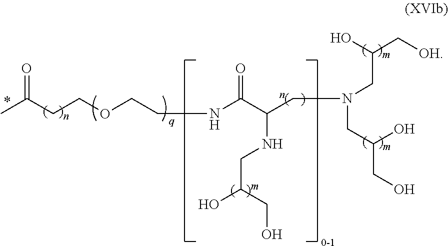

3. The Drug-Linker or salt of claim 2, wherein the at least one PEG unit has the formula

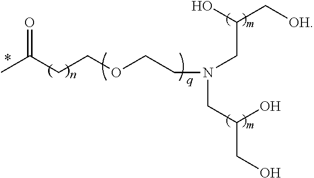

4. The Drug-linker or salt of claim 1, wherein q is independently 4-16.

5. The Drug-Linker or salt of claim 4, wherein q is 12.

6. The Drug-Linker or salt of claim 1, wherein m is 4.

7. The Drug-linker or salt of claim 1, wherein n is 1.

8. The Drug-Linker or salt of claim 1, wherein the Stretcher unit is capable of forming a bond with a sulfur atom.

9. The Drug-Linker or salt of claim 8, wherein the Stretcher unit comprises maleimido(C₁-C₁₀alkylene)-C(O)—, maleimido(CH₂OCH₂)ₚ₂(C₁-C₁₀alkyene)C(O), maleimido(C₁-C₁₀ alkyene)(CH₂OCH₂)ₚ₂C(O), or a ring open form thereof, wherein p2 is from 1 to 26.

10. The Drug-Linker or salt of claim 9, wherein the Stretcher unit comprises maleimido(C₁-C₁₀ alkylene)-C(O)—.

11. The Drug-Linker or salt of claim 1, wherein the Stretcher unit is

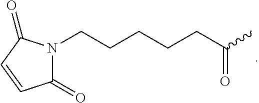

12. The Drug-Linker or salt of claim 1, wherein s is 0.

13. The Drug-Linker or salt of claim 1, wherein the cleavable peptide comprises a valine-citrulline peptide, a valine-alanine peptide, a valine-lysine peptide, a phenylalanine-lysine peptide, or a glycine-glycine-phenyalanine-glycine peptide.

14. The Drug-Linker or salt of claim 13, wherein the cleavable peptide comprises a Lys(PEG)-valine-citrulline peptide, a valine-Cit(PEG) peptide, a Lys(PEG)-valine-lysine peptide, a valine-lysine(PEG) peptide, a Lys(PEG)-valine-alanine peptide, a Lys(PEG)-phenylalanine-lysine peptide, a phenylalanine-Lys(PEG) peptide or a Lys(PEG)-glycine-glycine-phenylalanine-glycine peptide (SEQ ID NO: 46), wherein Lys(PEG) and Cit(PEG) comprise a PEG unit attached to a lysine residue or a citrulline residue, respectively, wherein the PEG unit is represented by the Formula (XVIb).

15. The Drug-Linker or salt of claim 1, wherein the cleavable peptide comprises a self immolative group.

16. The Drug-Linker or salt of claim 1, wherein the cleavable peptide comprises a para-aminobenzyl alcohol self immolative group (PABA) or a β-amino-benzyloxycarbonyl self immolative group.

17. The Drug-Linker or salt of claim 1, wherein the cleavable peptide comprises a β-amino-benzyloxycarbonyl self immolative group.

18. The Drug-Linker or salt of claim 17, wherein the cleavable peptide is attached to the Drug unit via the p-amino-benzyloxycarbonyl self immolative group.

19. The Drug-Linker or salt of claim 1, wherein s is 1.

20. The Drug-Linker or salt of claim 1, wherein the subunits of the Amino Acid unit are selected from alanine, arginine, aspartic acid, asparagine, histidine, glycine, glutamic acid, glutamine, phenylalanine, lysine, leucine, serine, tyrosine, threonine, isoleucine, proline, tryptophan, valine, ornithine, penicillamine, β-alanine, aminoalkanoic acid, aminoalkynoic acid, amino alkanedioic acid, aminobenzoic acid, amino-heterocyclo-alkanoic acid, heterocyclo-carboxylic acid, citrulline, and diaminoalkanoic acid; wherein the at least one PEG unit is attached to one of the subunits.

21. The Drug-Linker or salt of claim 19, wherein the at least one PEG unit has the formula selected from:

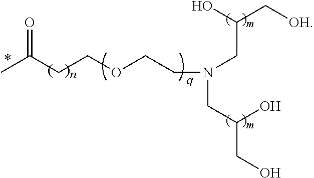

22. The Drug-Linker or salt of claim 1, wherein the Stretcher unit is selected from 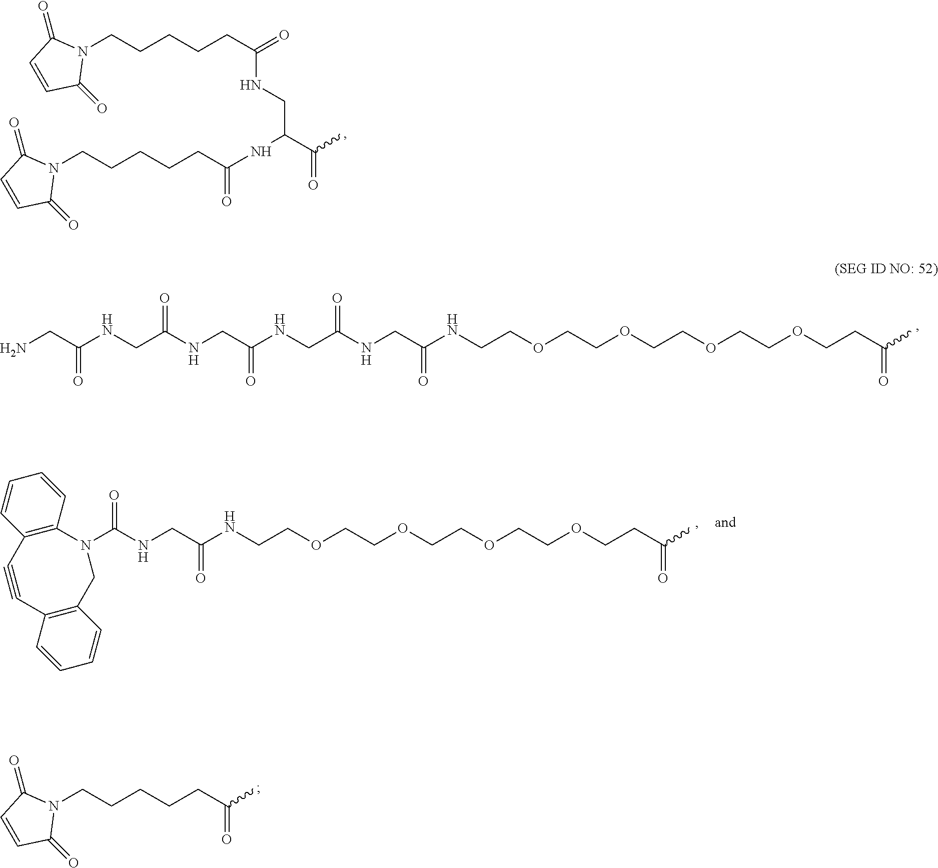
wherein the wavy line [symbol] indicates an attachment site of the Stretcher unit to the Amino Acid unit if s is 1 or L2 if s is 0.

23. The Drug-Linker or salt of claim 1, wherein the Drug unit is selected from a cytotoxic agent.

24. The Drug-Linker or salt of claim 23, wherein the cytotoxic agent is MMAE, MMAF, exatecan or SN-38.

25. The Drug-Linker or salt of claim 23, wherein the cytotoxic agent is exatecan.

26. The Drug-Linker or salt of claim 1, wherein the Drug-Linker is selected from:

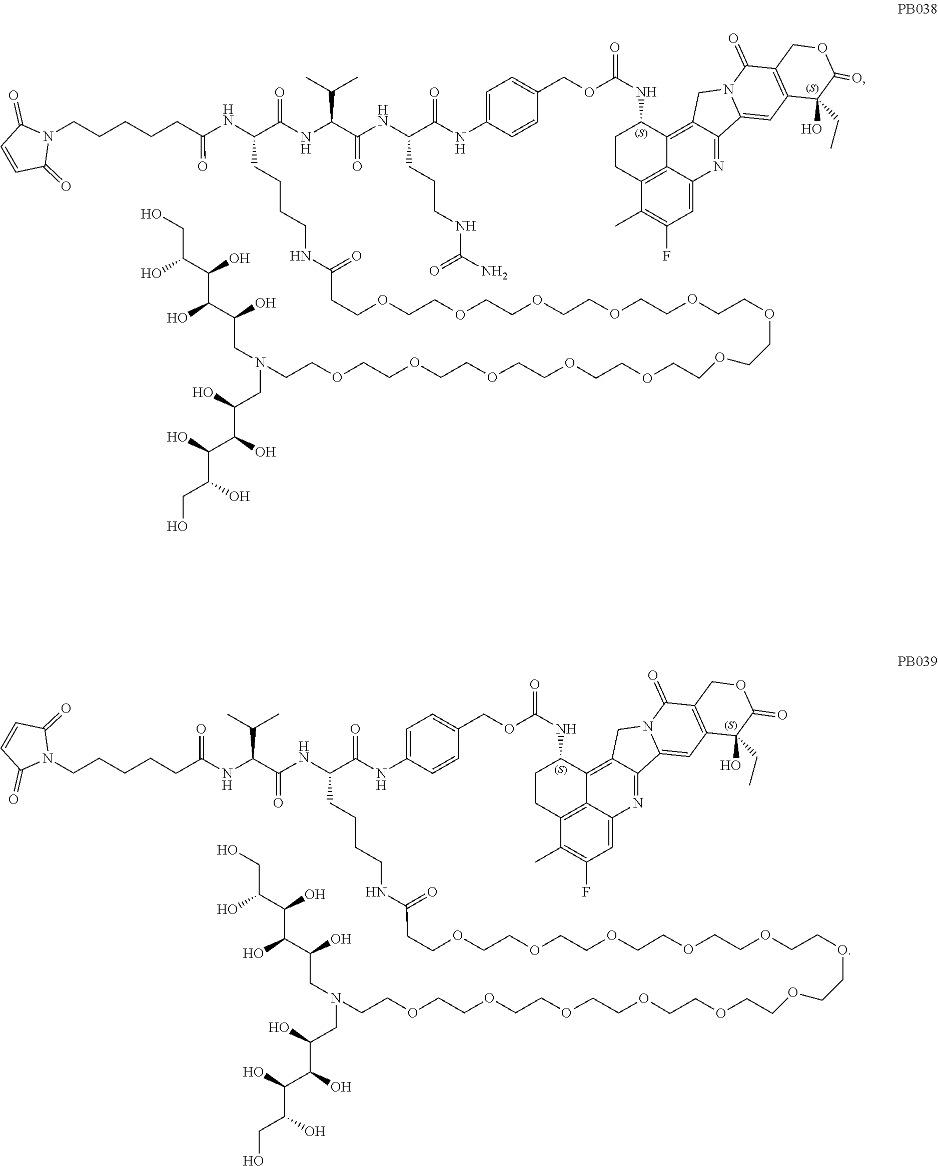

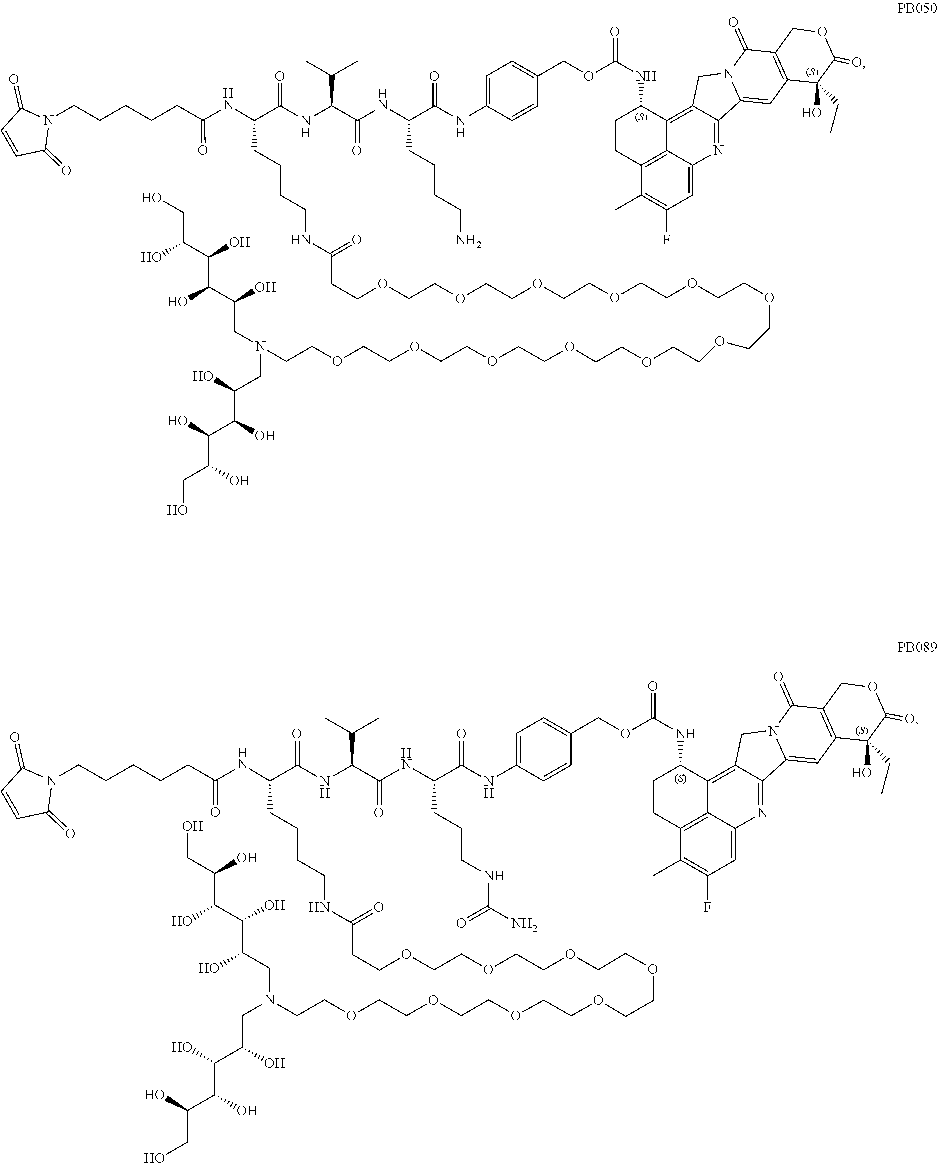

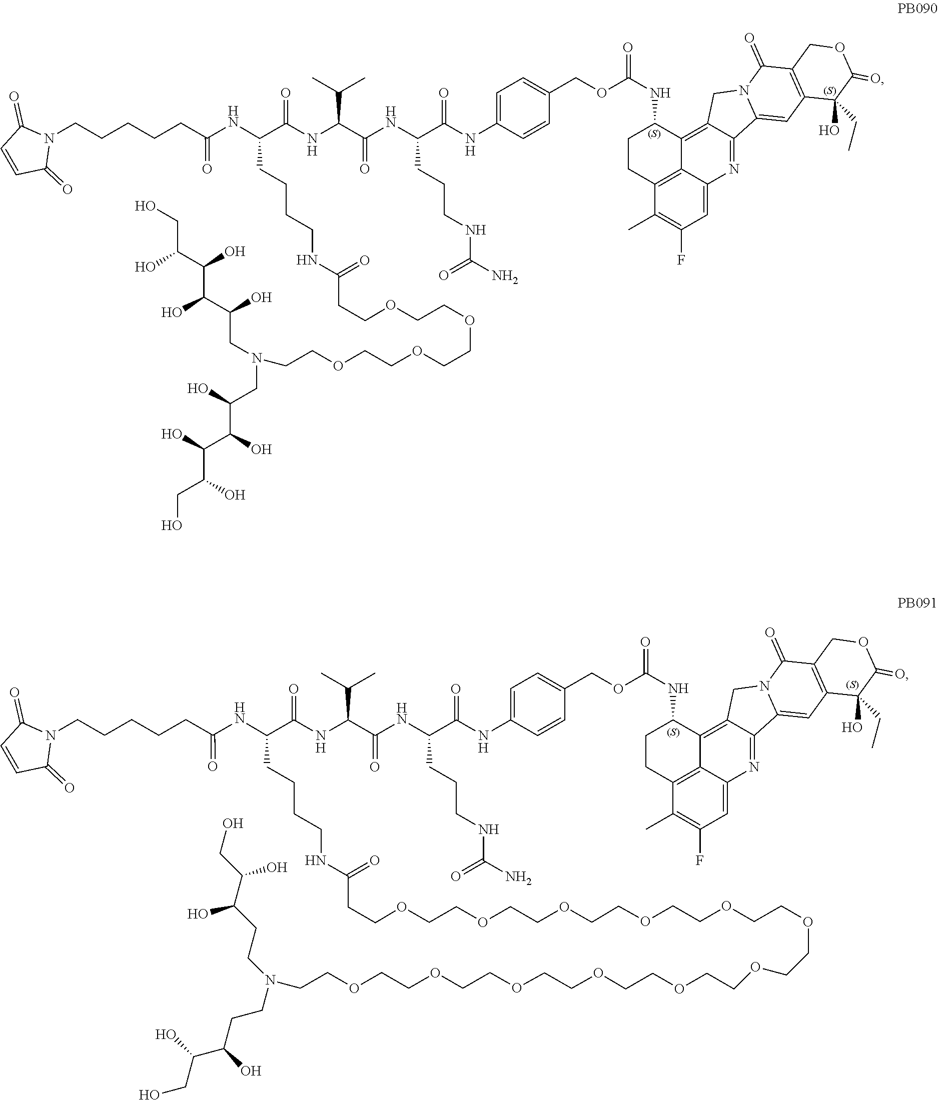

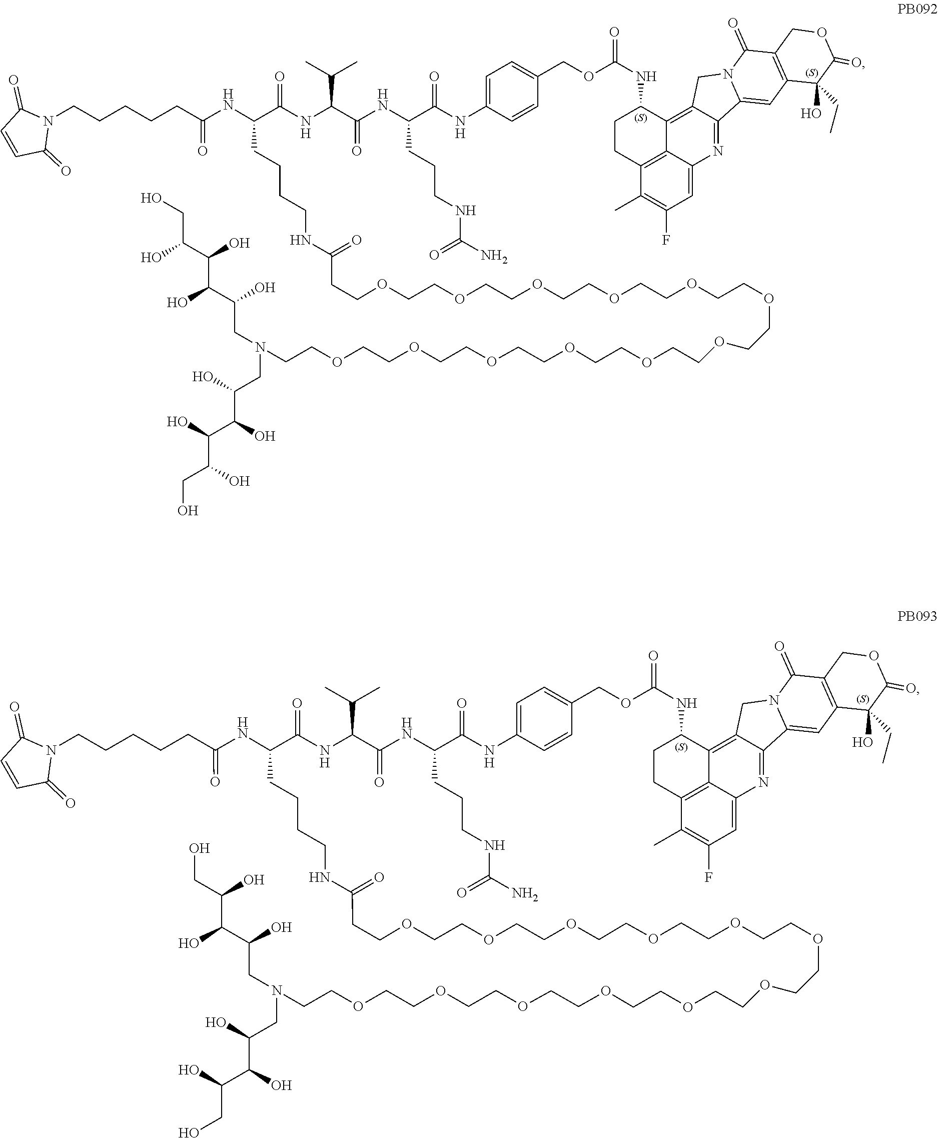

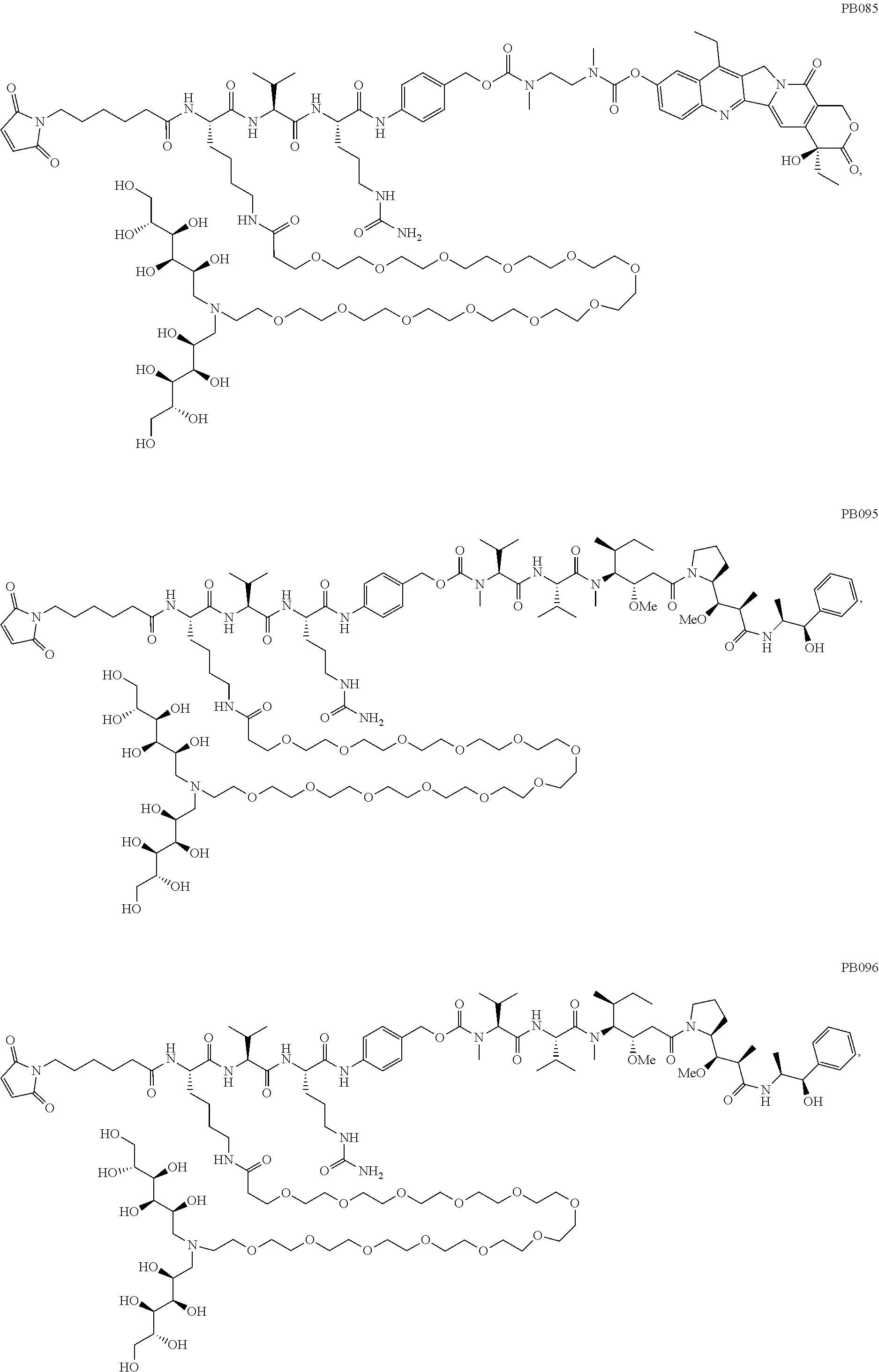

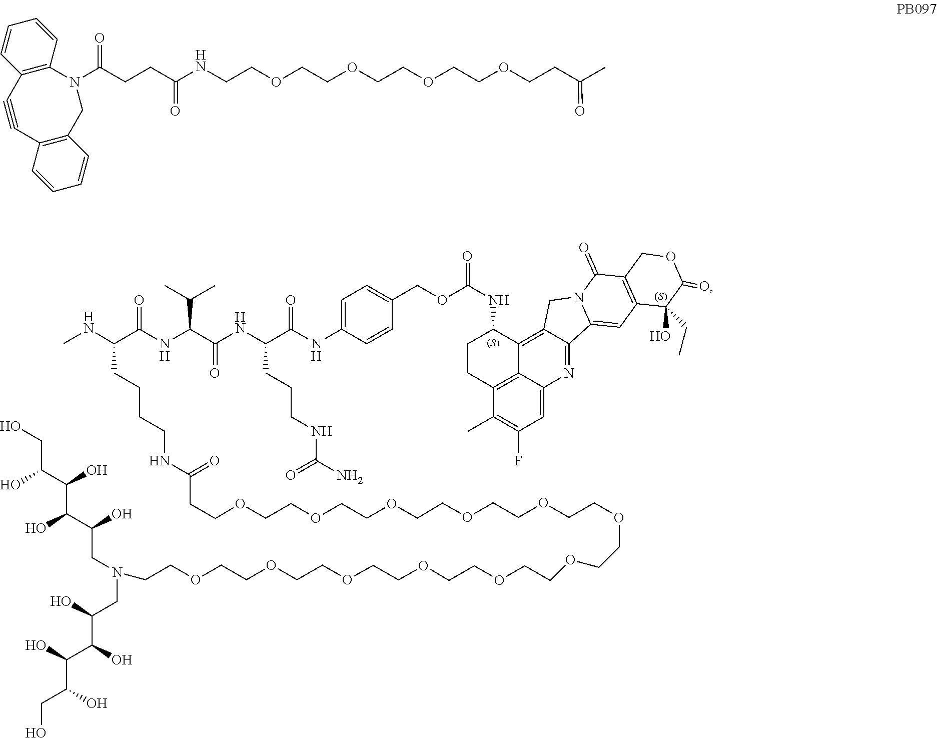

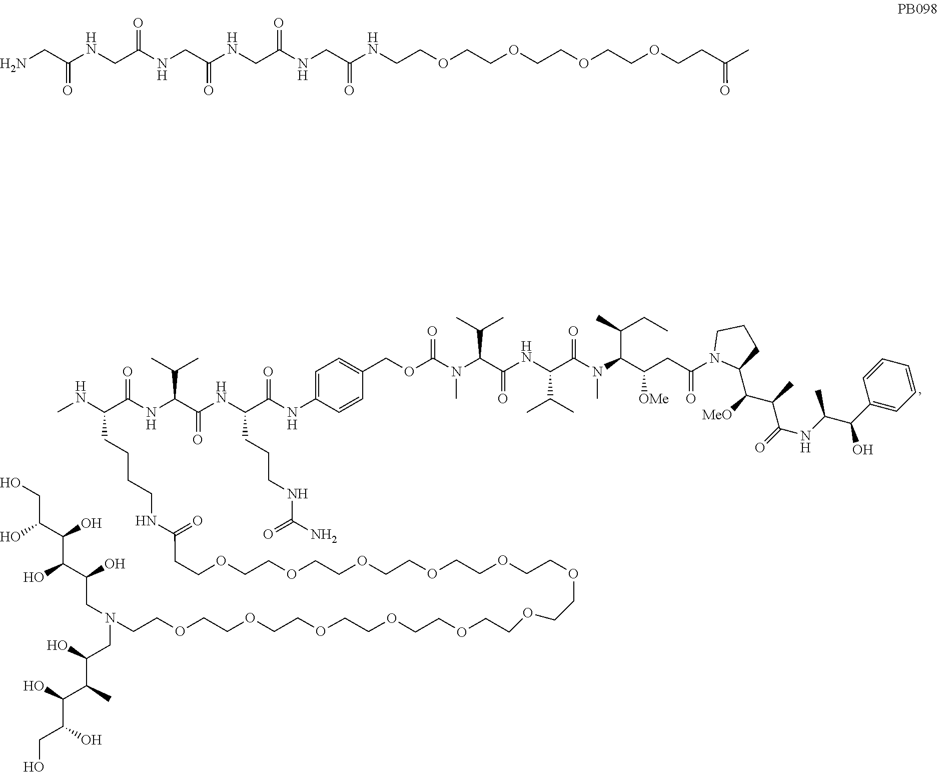 [SMILES: CC(=O)CCOCCOCCOCCOCCNC(=O)CNC(=O)CNC(=O)CNC(=O)CNC(=O)CN ; CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCCNC(=O)CCOCCOCCOCCOCCOCCOCCOCCOCCOCCOCCOCCOCCN(C[C@H](O)[C@@H](C)[C@H](O)[C@H](O)CO)C[C@H](O)[C@@H](O)[C@H](O)[C@H](O)CO)NC)C(C)C)cc1)C(C)C]

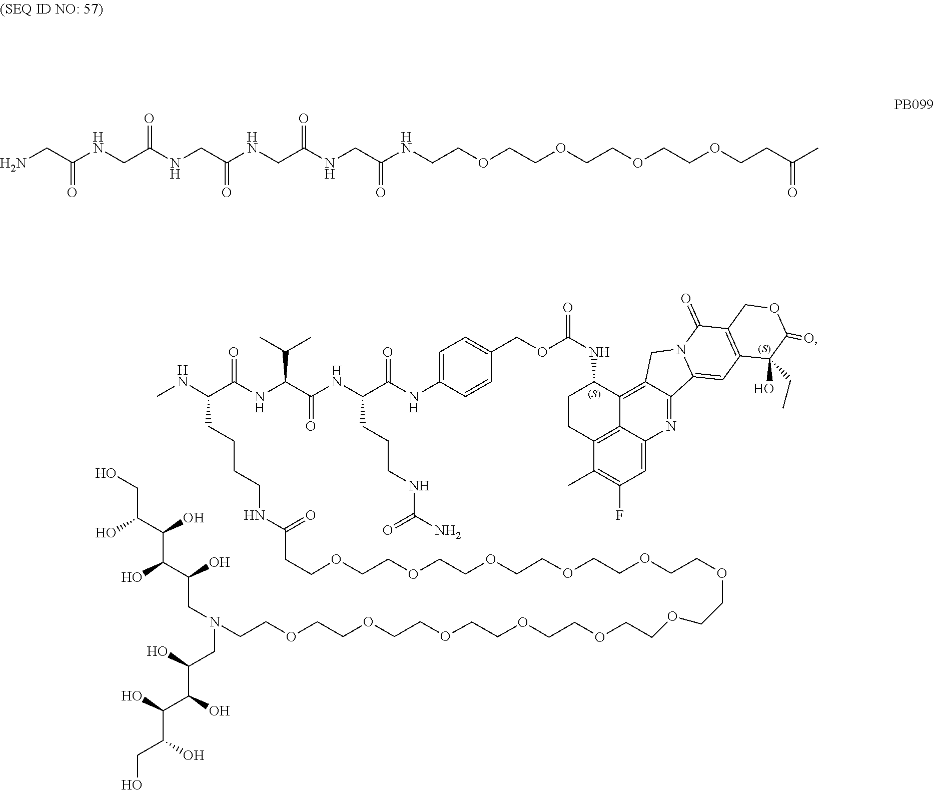

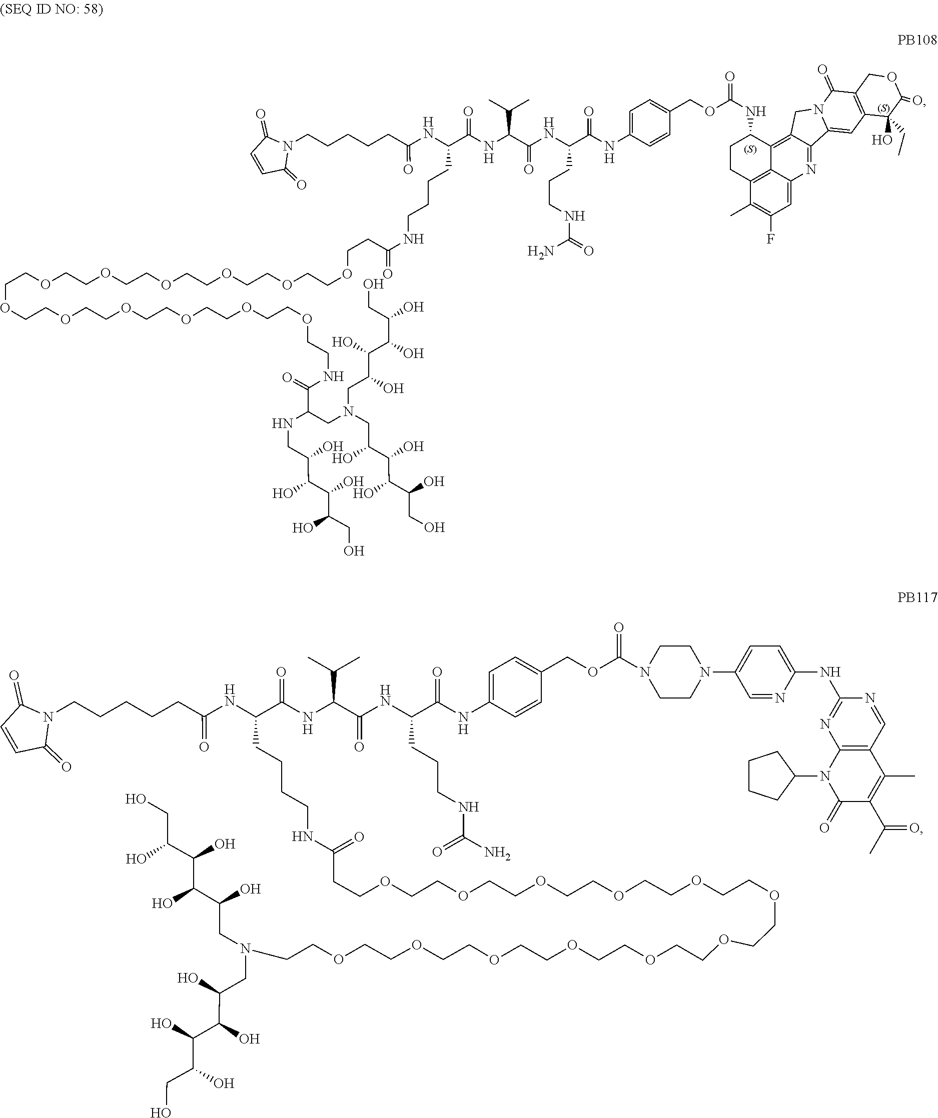

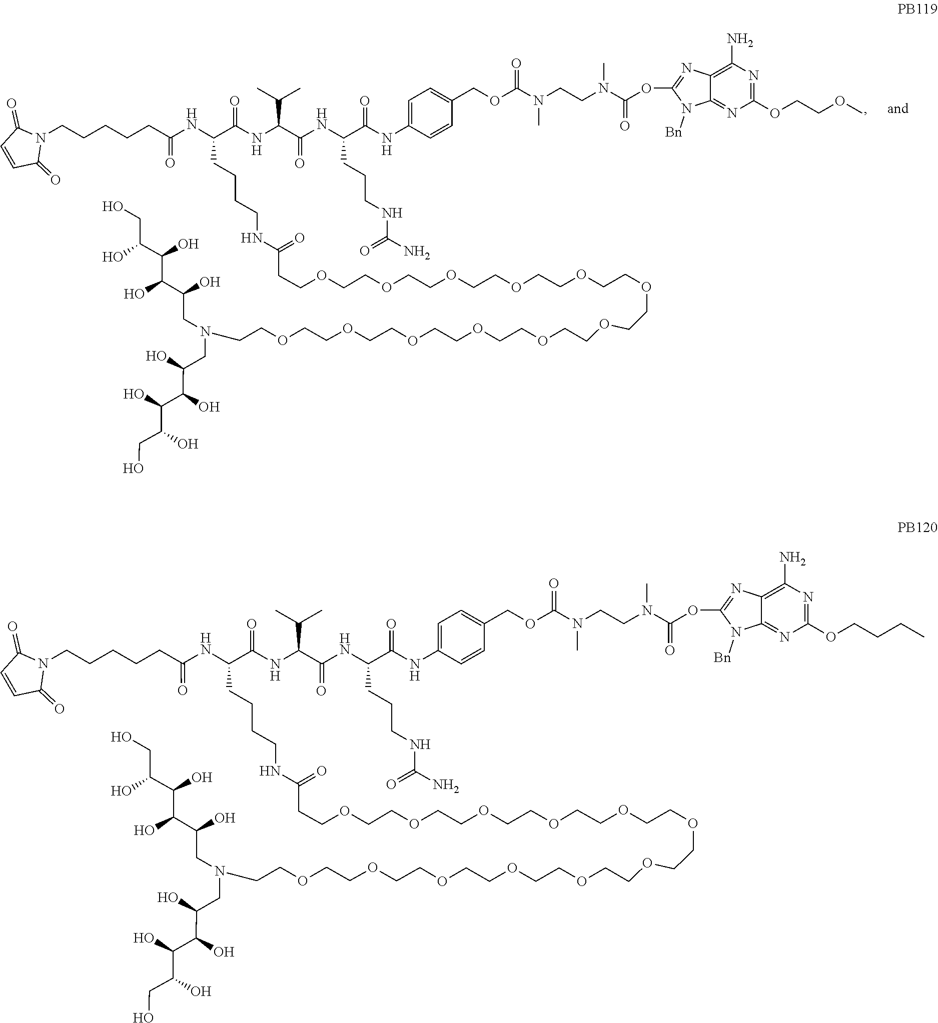

27. The Drug-Linker or salt of claim 1, wherein the Drug-Linker is selected from: 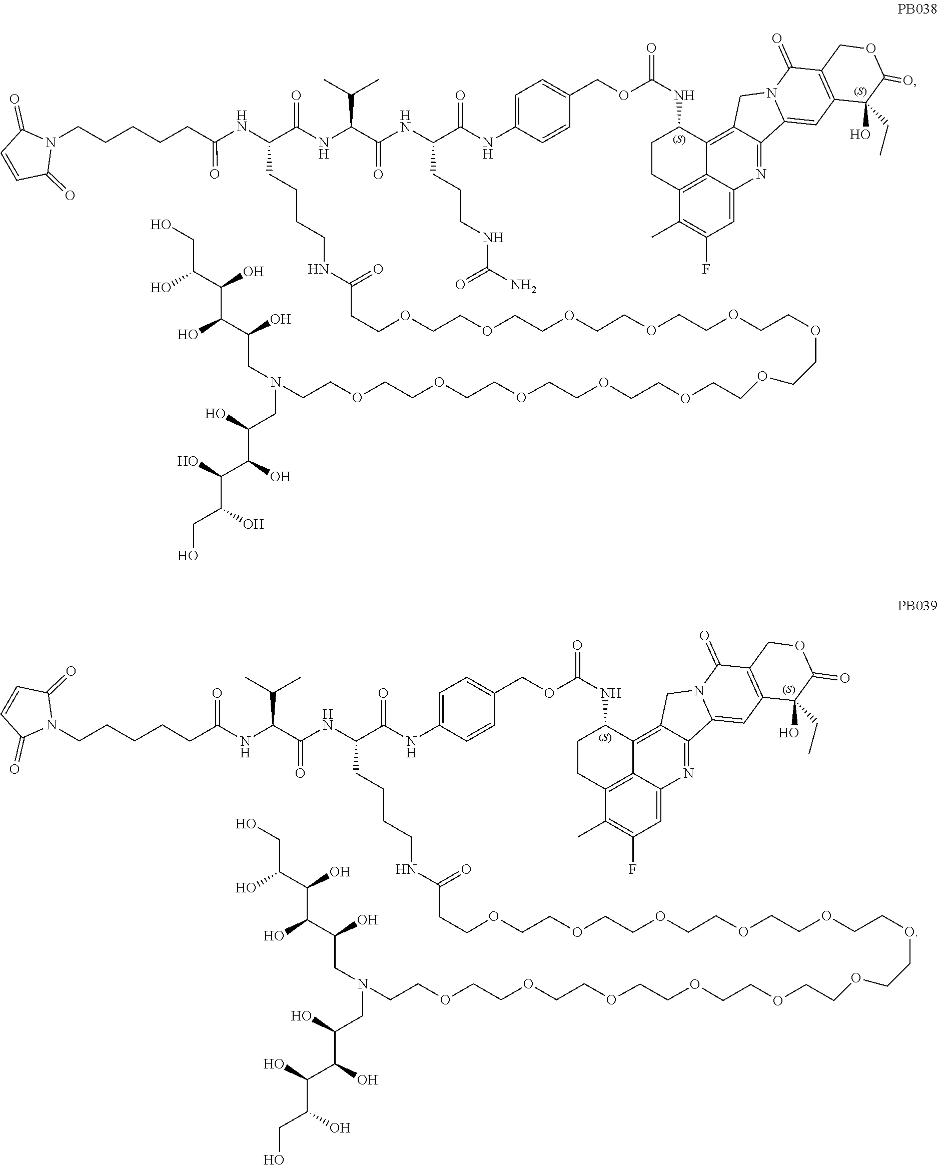 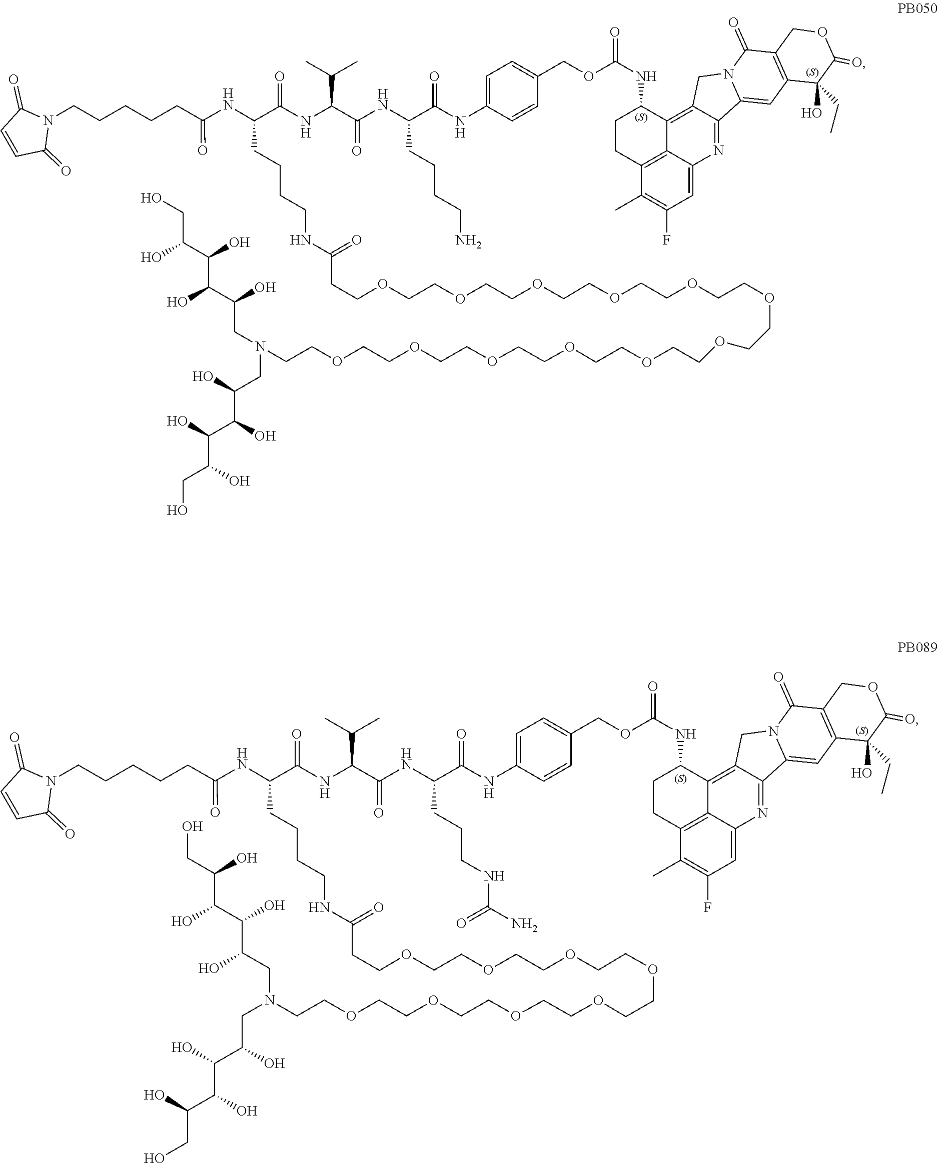 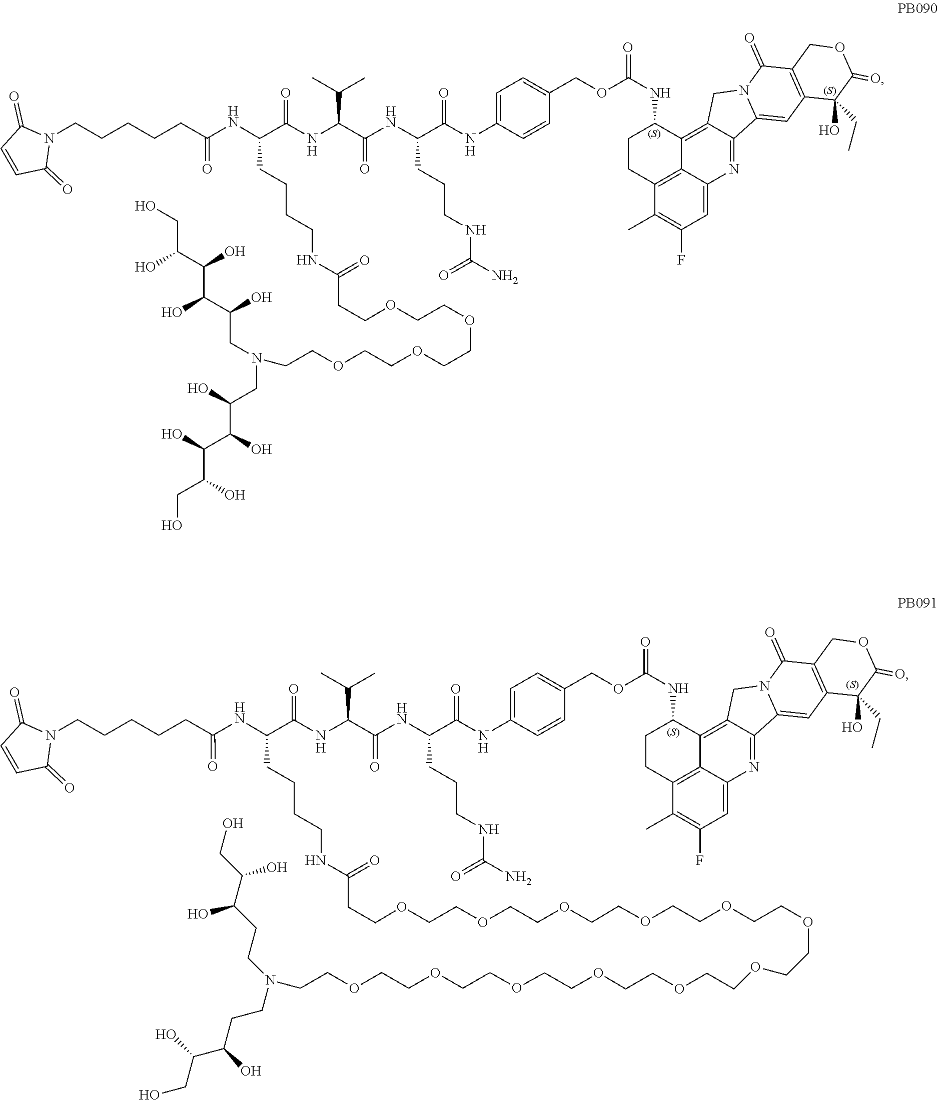 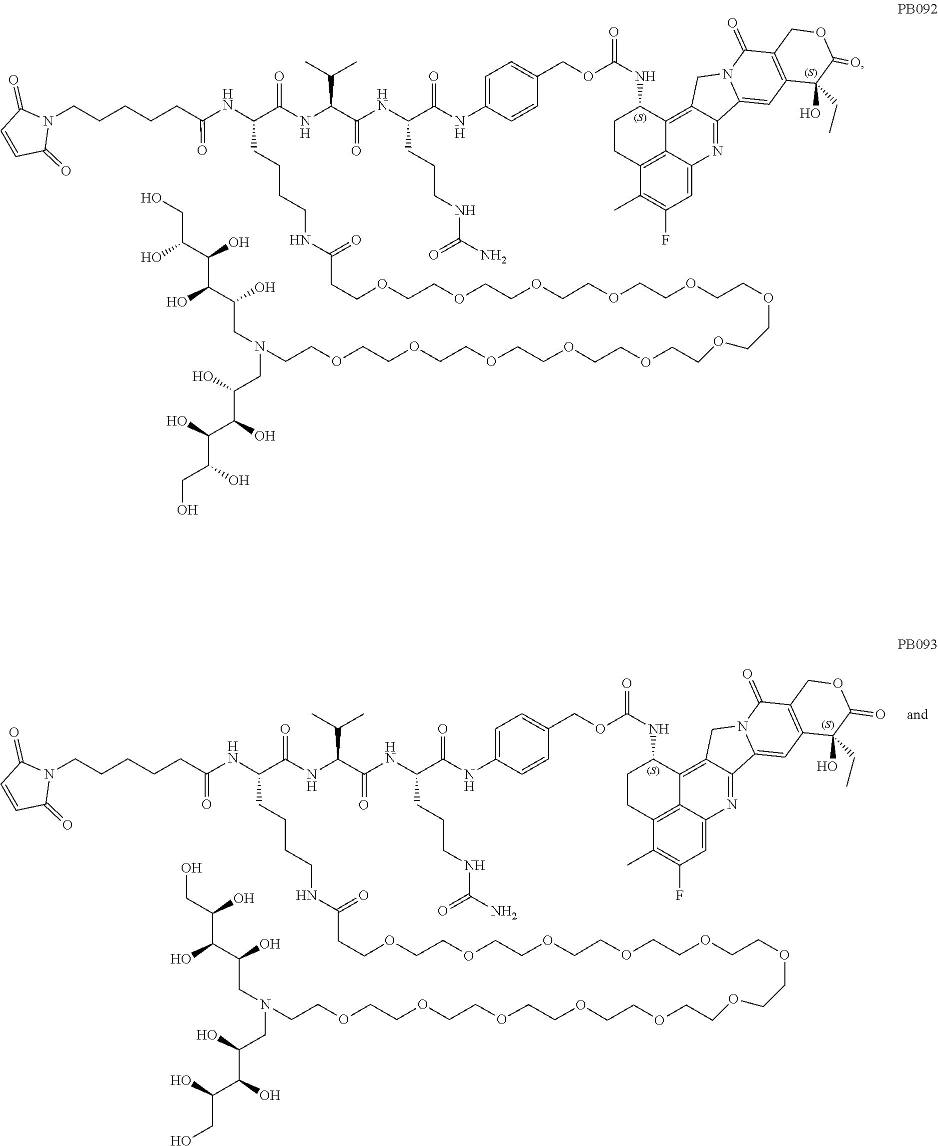 [chemical structure]
